(19) United States
(12) Patent Application Publication
(10) Pub. No.: US 2025/0304563 A1
(43) Pub. Date: Oct. 2, 2025

(54) ACTIVATORS OF EFFECTOR T CELLS

(71) Applicant: Orum Therapeutics, Inc., Yuseong-gu (KR)

(72) Inventors: Peter U. PARK, Cambridge, MA (US); Nathan FISHKIN, Cambridge, MA (US); Chen BAI, Arlington, MA (US); Khuloud TAKROURI, Wayland, MA (US); Zinaida RIBKOVSKAIA, Ayer, MA (US); James Joseph PALACINO, Wellesley, MA (US); Wesley WONG, Cambridge, MA (US); Anna SKALETSKAYA, Arlington, MA (US); Suk NAMGOONG, Cheongju (KR); Joanne Y. H. LIM, Natick, MA (US)

(21) Appl. No.: 18/845,298

(22) PCT Filed: Mar. 8, 2023
(86) PCT No.: PCT/IB2023/052217

§ 371 Date: Sep. 9, 2024

Related U.S. Application Data
(60) Provisional application No. 63/318,278, filed on Mar. 9, 2022.
(60) Provisional application No. 63/477,053, filed on Dec. 23, 2022.
(60) Provisional application No. 63/488,211, filed on Mar. 3, 2023.

(51) Int. Cl. C07D 405/14; A61K 9/00; A61K 31/454; A61K 38/00; A61K 45/06; A61K 47/68; A61P 35/00; C07D 401/14

(52) U.S. Cl. CPC C07D 405/14; A61K 9/0019; A61K 31/454; A61K 38/00; A61K 45/06; A61K 47/6803; A61K 47/6849; A61K 47/6851; A61P 35/00; C07D 401/14

(57) ABSTRACT

The present disclosure provides antibody drug conjugates and methods of delivering the conjugates to effector T cells. Also disclosed are novel Cbl-b inhibitors. The compounds and conjugates are useful for treating diseases in subjects in need thereof.

### CROSS-REFERENCE TO RELATED APPLICATIONS

This Application claims the priority benefit of U.S. Provisional Application No. 63/318,278, filed Mar. 9, 2022; U.S. Provisional Application No. 63/477,053, filed Dec. 23, 2022; and U.S. Provisional Application No. 63/488,211, filed Mar. 3, 2023, which are incorporated herein by reference in their entireties.

### REFERENCE TO SEQUENCE LISTING SUBMITTED ELECTRONICALLY

The content of the electronically submitted sequence listing in .XML file (Name: 4547_023PC03_SeqListing_ST26.xml; Size: 42,283 bytes; and Date of Creation: Mar. 8, 2023), filed with the application, is incorporated herein by reference in its entirety.

### FIELD

The present disclosure provides antibody drug conjugates and methods of delivering the conjugates to effector T cells. Also disclosed are novel Cbl-b inhibitors. The compounds and conjugates are useful for treating diseases in subjects in need thereof.

### BACKGROUND

Effector T cells are major participants in steering the immune system to execute immune functions. These cells circulate until they encounter their specific antigen. As such, they play a critical part in immunity. The immune system is a powerful weapon against many diseases and disorders, including e.g., cancer, and it has been shown that compounds that activate effector T cells, such as inhibitors of the E3 ubiquitin ligase Casitas B-lineage lymphoma-b (Cbl-b), increase T cell-derived cytockine secretion and proliferation and may have anti-cancer activity. However, improved inhibitors of Cbl-b are needed.

Furthermore, compounds that activate effector T cells have the potential to cause cytokine release syndrome or other off-target immune reactions. It has been proposed that such off-target immune reactions could be avoided by targeting immune-activating drugs specifically to cancer cells by conjugating the immune-activating drugs to antibodies that specifically recognize tumor antigens. (See e.g., Ackerman, S. E., Pearson, C. I., Gregorio, J. D. et al. Immune-stimulating antibody conjugates elicit robust myeloid activation and durable antitumor immunity. Nat Cancer 2, 18-33 (2021). doi.org/10.1038/s43018-020-00136-x.) Such proposals, however, do not suggest directly targeting the immune system itself. Therefore, despite such ongoing work, there is a need to activate T cells, in particular effector T cells, e.g., for the treatment of cancer, while minimizing off-target immune reactions.

### SUMMARY

In certain aspects, the present disclosure provides a compound of formula (I):

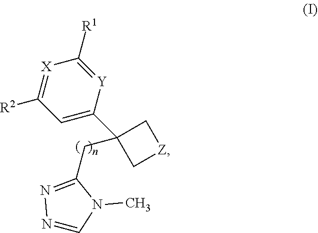

or a pharmaceutically acceptable salt thereof, wherein:
   n is 0, 1, or 2;
   X and Y are each independently CH or N;
   Z is selected from CH(CH₃), O, and SO₂; or
   Z is selected from CH(CH₃), NH, N(CH₃), O, and SO₂;
   R¹ is selected from hydrogen, —CN, —NHR^{z}, —R^{a}, —NR^{a}R^{b}, —OR^{a}, —NHC(O)R^{a}, —NHC(S)R^{a}, —NHC(O)NHR^{a}, —NHC(S)NHR^{a}, —SR^{a}, C₃-C₆cycloalkyl, and a 3- to 6-membered heterocyclyl ring; wherein:
      R^{z} is selected from

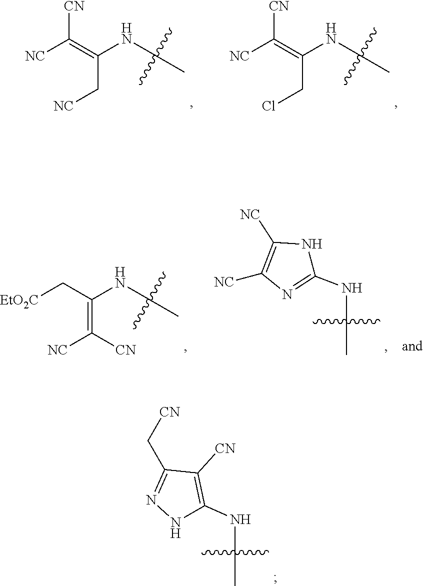 [SMILES: *NC(CC#N)=C(C#N)C#N ; *NC(CCl)=C(C#N)C#N ; *NC(CC(=O)OCC)=C(C#N)C#N ; *Nc1nc(C#N)c(C#N)[nH]1 ; *Nc1[nH]nc(CC#N)c1C#N]

R^{a} and R^{b} are independently selected from hydrogen, C₂-C₆alkenyl, C₁-C₆alkyl, amido(C₁-C₆alkyl), amino(C₁-C₆alkyl), azido(C₁-C₆alkyl), C₂-C₆alkynyl, carboxy(C₁-C₆alkyl), cyano(C₁-C₆alkyl), C₃-C₆cycloalkyl optionally substituted with a cyano group, dimethylamino(C₁-C₆alkyl), a 3- to 6-membered heterocyclyl ring, 3-6-membered heterocyclyl(C₁-C₃alkyl), hydroxy(C₁-C₆alkyl), methoxy(C₁-C₆alkyl), methylamino(C₁-C₆alkyl), NR^{c}R^{d}(C₁-C₆alkyl), HS(C₁-C₆alkyl), and CH₃S(C₁-C₆alkyl), wherein R^{c} and R^{d} are independently selected from hydrogen, C₂alkenylcarbonyl, and methyl; or,
R^{a} and R^{b}, together with the nitrogen atom to which they are attached, form a five- or six-membered ring optionally containing one additional nitrogen atom, wherein the ring is optionally substituted with one group selected from R^{a}, —C(O)R^{a}, —SO₂R^{a}, azido, and cyano;
wherein each C₃-C₆cycloalkyl, each 3- to 6-membered heterocyclyl ring, and the heterocyclyl part of the 3- to 6-membered heterocyclyl(C₁-C₃alkyl) ring are optionally substituted with one, two, or three groups independently selected from C₁-C₃alkyl, C₂alkynyl, amido, azido, carboxy, cyano, dimethylamino, hydroxy, methoxy, methylamino, HS—, and CH₃S—; and
R² is selected from

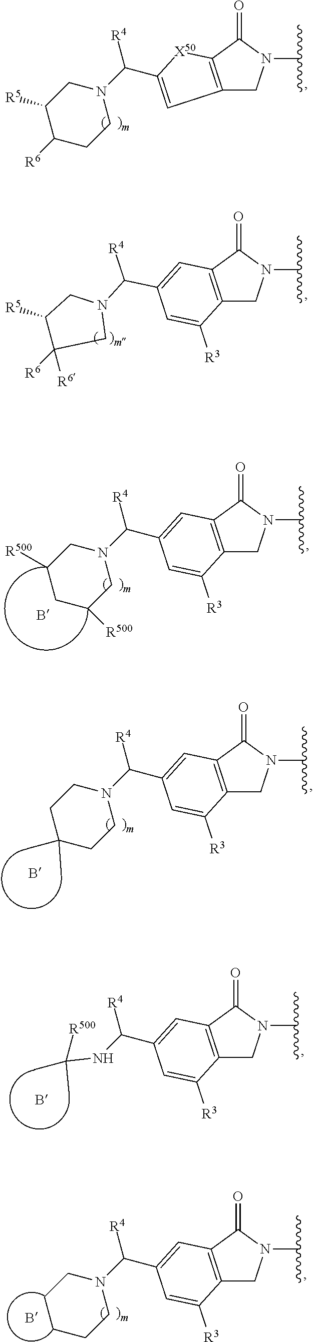

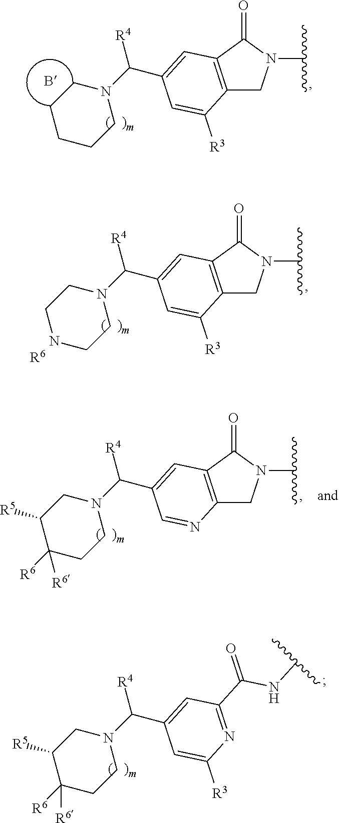

m is 0, 1, 2, or 3;
m″ is 0, 1, 2, 3, or 4;
B′ is a 3-7 membered saturated or unsaturated ring optionally containing one or two heteroatoms independently selected from nitrogen, oxygen, and sulfur; wherein the ring is optionally substituted with one or two substituents independently selected from —OH, —CH₂SH, CH₂SCH₃, —CH₂OH, —CH₂NH₂, and —CH₂NHCH₃;
each R⁵⁰⁰ is independently selected from hydrogen, C₁-C₆alkyl, halo, —OH, and —CH₂OH; and
X⁵⁰ is selected from O, NH, NCH₃, and S;
R³ is selected from hydrogen, acetyl, amino, C₁-C₆alkylamino, C₁-C₆alkylaminomethyl, C₁-C₆alkylcarbonyl, aminoC₁-C₆alkyl, aminocarbonyl, aminomethyl, carboxy, cyano, C₃cycloalkyl, formyl, hydroxy, hydroxyC₁-C₆alkyl, methoxy, oxazolyl, —SH, —SCH₃, —SOCH₃, —SO₂CH₃, —SO(═NH)CH₃, tetrazolyl, thiazolyl, and trifluoromethyl, wherein the C₃cycloalkyl is optionally substituted with a hydroxy group;
R⁴ is selected from hydrogen, methyl, —CH₂OH, —CH₂SH, and —CH₂SCH₃;
R⁵ is selected from hydrogen, hydroxy, —CH₂SH, —CH₂SCH₃, and methyl;
optionally provided that when R⁵ is hydroxy or methyl, and R⁴ is hydrogen, then R¹ is other than C₃-C₆cycloalkyl, a 3- to 6-membered heterocyclyl ring, hydroxy, hydroxy(C₁-C₆alkyl), —OR^{a} wherein R^{a} is C₁-C₆alkyl, a 3- to 6-membered heterocyclyl ring, or hydroxy(C₁-C₆alkyl); or —NR^{a}R^{b}, wherein R^{a} and R^{b} are independently selected from the group consisting of hydrogen, C₁-C₆alkyl, C₃-C₆cycloalkyl, hydroxy(C₁-C₆alkyl), a 3- to 6-membered heterocyclyl ring, or wherein R^{a} and R^{b}, together with the nitrogen atom to which they are attached, form a five- or six-membered ring optionally containing one additional nitrogen atom, wherein the ring is optionally substituted with one group selected from C₁-C₆alkyl or hydroxy(C₁-C₆alkyl); and
R⁶ and R^{6′} are independently selected from hydrogen, cyclopropyl, —CH₂OH, —CH₂SH, —CH₂SCH₃, and —CH₂R²⁰⁰, wherein R²⁰⁰ is a 3-7 membered saturated or unsaturated ring optionally containing one or two heteroatoms independently selected from nitrogen, oxygen, and sulfur.

In some aspects, R⁴ is in the “R” configuration. In some aspects, R⁴ is in the “S” configuration.

In certain aspects, the present disclosure provides a compound of formula (IA-1):

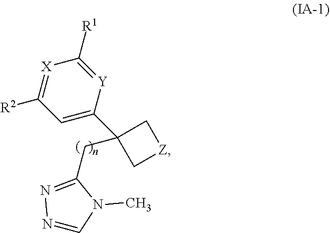

or a pharmaceutically acceptable salt thereof, wherein:
   n is 0, 1, or 2;
   X and Y are each independently CH or N;
   Z is selected from CH(CH₃), O, and SO₂;
   R¹ is selected from hydrogen, —CN, —NHR^{z}, —R^{a}, —NR^{a}R^{b}, —OR^{a}, —NHC(O)R^{a}, —NHC(S)R^{a}, —NHC(O)NHR^{a}, —NHC(S)NHR^{a}, —SR^{a}, C₃-C₆cycloalkyl, and a 3- to 6-membered heterocyclyl ring; wherein:
      R^{z} is selected from

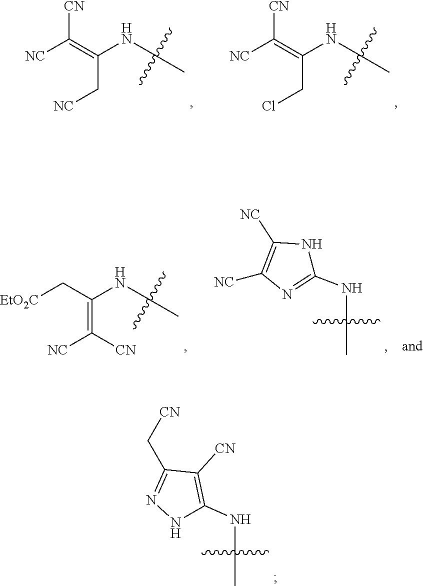 [SMILES: *NC(CC#N)=C(C#N)C#N ; *NC(CCl)=C(C#N)C#N ; *NC(CC(=O)OCC)=C(C#N)C#N ; *Nc1nc(C#N)c(C#N)[nH]1 ; *Nc1[nH]nc(CC#N)c1C#N]

R^{a} and R^{b} are independently selected from hydrogen, C₂-C₆alkenyl, C₁-C₆alkyl, amido(C₁-C₆alkyl), amino(C₁-C₆alkyl), azido(C₁-C₆alkyl), C₂-C₆alkynyl, carboxy(C₁-C₆alkyl), cyano(C₁-C₆alkyl), C₃-C₆cycloalkyl, dimethylamino(C₁-C₆alkyl), a 3- to 6-membered heterocyclyl ring, 3-6-membered heterocyclyl(C₁-C₃alkyl), hydroxy(C₁-C₆alkyl), methoxy(C₁-C₆alkyl), methylamino(C₁-C₆alkyl), NR^{c}R^{d}(C₁-C₆alkyl), HS(C₁-C₆alkyl), and CH₃S(C₁-C₆alkyl), wherein R^{c} and R^{d} are independently selected from hydrogen, C₂alkenylcarbonyl, and methyl; or,
R^{a} and R^{b}, together with the nitrogen atom to which they are attached, form a five- or six-membered ring optionally containing one additional nitrogen atom, wherein the ring is optionally substituted with one group selected from R^{a}, —C(O)R^{a}, —SO₂R^{a}, azido, and cyano;
wherein each C₃-C₆cycloalkyl, each 3- to 6-membered heterocyclyl ring, and the heterocyclyl part of the 3- to 6-membered heterocyclyl(C₁-C₃alkyl) ring are optionally substituted with one, two, or three groups independently selected from C₁-C₃alkyl, C₂alkynyl, amido, azido, carboxy, cyano, dimethylamino, hydroxy, methoxy, methylamino, HS—, and CH₃S—; and

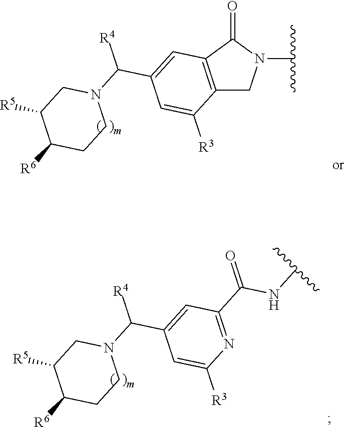

m is 0, 1, 2, or 3;
R³ is selected from hydrogen, acetyl, amino, C₁-C₆alkylamino, C₁-C₆alkylaminomethyl, aminoC₁-C₆alkyl, aminocarbonyl, aminomethyl, carboxy, cyano, C₃cycloalkyl, formyl, hydroxy, hydroxyC₁-C₆alkyl, methoxy, oxazolyl, —SH, —SCH₃, tetrazolyl, thiazolyl, and trifluoromethyl, wherein the C₃cycloalkyl is optionally substituted with a hydroxy group;
R⁴ and R⁶ are independently selected from hydrogen, —CH₂SH, and —CH₂SCH₃; and
R⁵ is selected from hydroxy, —CH₂SH, —CH₂SCH₃, and methyl;
optionally provided that when R⁵ is hydroxy or methyl, and R⁴ is hydrogen, then R¹ is other than C₃-C₆cycloalkyl, a 3- to 6-membered heterocyclyl ring, hydroxy, hydroxy(C₁-C₆alkyl), —OR^{a} wherein R^{a} is C₁-C₆alkyl, a 3- to 6-membered heterocyclyl ring, or hydroxy(C₁-C₆alkyl); or —NR^{a}R^{b}, wherein R^{a} and R^{b} are independently selected from the group consisting of hydrogen, C₁-C₆alkyl, C₃-C₆cycloalkyl, hydroxy(C₁-C₆alkyl), a 3- to 6-membered heterocyclyl ring, or wherein R^{a} and R^{b}, together with the nitrogen atom to which they are attached, form a five- or six-membered ring optionally containing one additional nitrogen atom, wherein the ring is optionally substituted with one group selected from C₁-C₆alkyl or hydroxy(C₁-C₆alkyl),

In some aspects, R⁴ is in the “R” configuration. In some aspects, R⁴ is in the “S” configuration.

In certain aspects, the present disclosure provides a compound of formula (I), or a pharmaceutically acceptable salt thereof, wherein:
X is N;
Y is CH; and
R² is

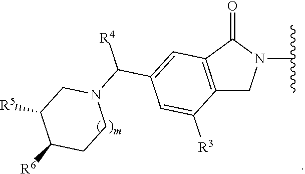

In some aspects, R⁴ is in the “R” configuration. In some aspects, R⁴ is in the “S” configuration.

In certain aspects, Z is O. In some aspects, Z is CH(CH₃).

In some aspects, the present disclosure provides a compound of formula (I), or a pharmaceutically acceptable salt thereof, wherein
R³ is trifluoromethyl;
R⁴ and R⁶ are hydrogen; and
R⁵ is methyl.

In certain aspects, R¹ is selected from —NR^{a}R^{b}, —NHC(O)R^{a}, —NHC(S)NHR^{a}, and —SR^{a}. In some aspects, wherein R¹ is —SCH₂CH₃. In some aspects, R¹ is —NH(CH₂)₂CN. In some aspects, R¹ is —NH(CH₂)₂N₃.

In some aspects, the present disclosure provides a compound of formula (I), or a pharmaceutically acceptable salt thereof, selected from

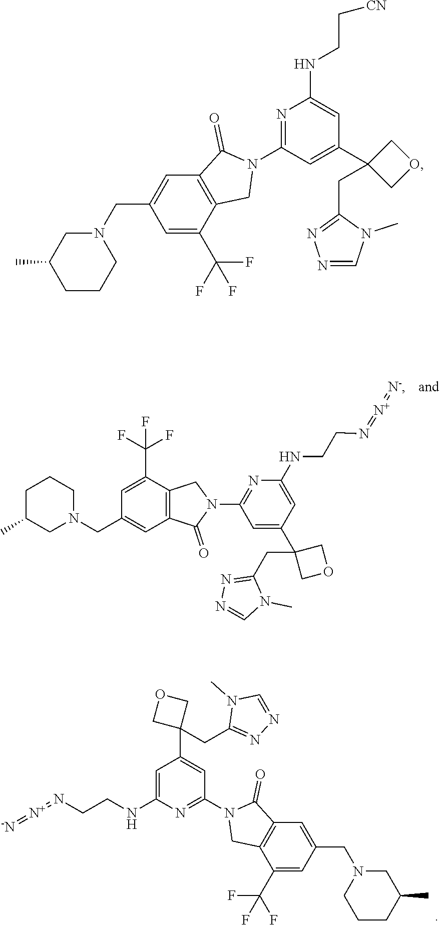

In some aspects, the compound of formula (I) is an inhibitor of Casitas B-lineage lymphoma proto-oncogene b (Cbl-b).

In certain aspects, the compound interacts with His152 of human Cbl-b; increases IL-2 secretion from T cells over background by about 0.8 to about 1.4 fold; increases IFN-y secretion over background by about 1.1 to about 2 fold; and/or increases CD69 levels over background by about 0.81 to about 1.1 fold.

In certain aspects, the present disclosure provides a conjugate, or a pharmaceutically acceptable salt thereof, comprising a binding moiety that is capable of specifically binding to a target on the surface of an effector T cell and a payload that is capable of activating an effector T cell, wherein the binding moiety is directly attached to the payload or is attached to the payload through a linker. In some aspects, the binding moiety is attached to the payload through a linker. In certain aspects, the binding moiety is capable of specifically binding a protein or glycoprotein on the surface of the effector T cell. In some aspects, the binding moiety is capable of specifically binding to programmed cell death protein 1 (PD1).

In certain aspects, the present disclosure provides a conjugate, or a pharmaceutically acceptable salt thereof, comprising a binding moiety that is capable of specifically binding to PD1 and a payload that is capable of activating an effector T cell, wherein the binding moiety is directly attached to the payload or is attached to the payload through a linker.

In some aspects, the present disclosure provides a conjugate, or a pharmaceutically acceptable salt thereof, comprising a binding moiety that is capable of specifically binding to PD1 and a payload that is an inhibitor of Casitas B-lineage lymphoma proto-oncogene b (Cbl-b).

In some aspects, the conjugate described herein has formula (I):

Bm-[L-P]ₐ (I),
wherein:
   a is an integer from 1 to 50;
   P is the payload;
   L is a linker; and
   Bm is the binding moiety.

In some aspects, the payload is an inhibitor of Casitas B-lineage lymphoma proto-oncogene b (Cbl-b). In some aspects, the inhibitor of Cbl-b is a compound of formula (I), which is attached to the binding moiety or the linker through a covalent bond. In some aspects, the inhibitor of Cbl-b is

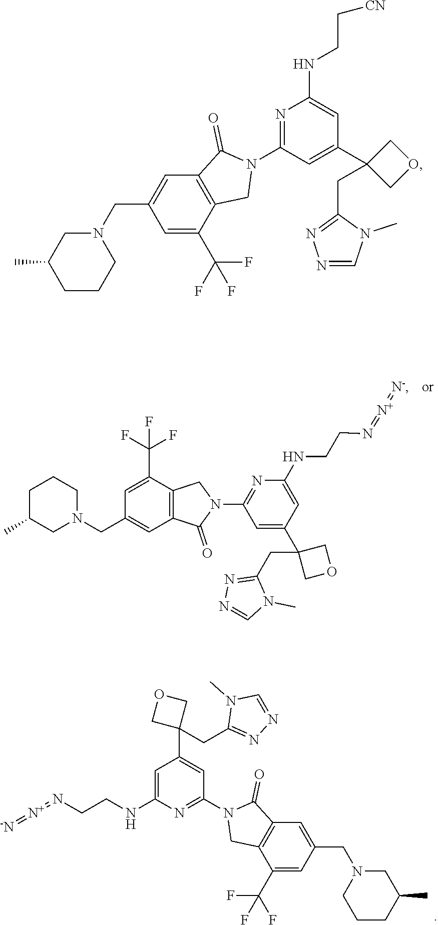

In some aspects, the inhibitor of Cbl-b is Compound 146, Compound 147, Compound 148, or NX-1607.

In some aspects, the payload is an agonist of toll-like receptor 7 (TLR-7) and/or toll-like receptor 8 (TLR-8). In some aspects, the payload is the TLR-7/TLR-8 agonist T785. In some aspects, the payload is the TLR-7/TLR-8 agonist MEDI9197.

In some aspects, the payload is an inhibitor of hematopoietic progenitor kinase 1 (HPK-1).

In some aspects, the payload is an inhibitor of STING, phosphoinositide-3-kinase gamma (PI3K7), CXCR4, CCR5, or a mitogen-activated protein kinase (MAPK) pathway protein, optionally wherein the MAPK pathway protein is MEK or B-raf.

In some aspects, the payload is an agonist of stimulator of interferon genes (STING).

In some aspects, the payload is a small molecule. In some aspects, the payload is a peptide.

In certain aspects, the present disclosure provides a conjugate as described above wherein L is a non-cleavable linker. In some aspects, L is selected from the group consisting of

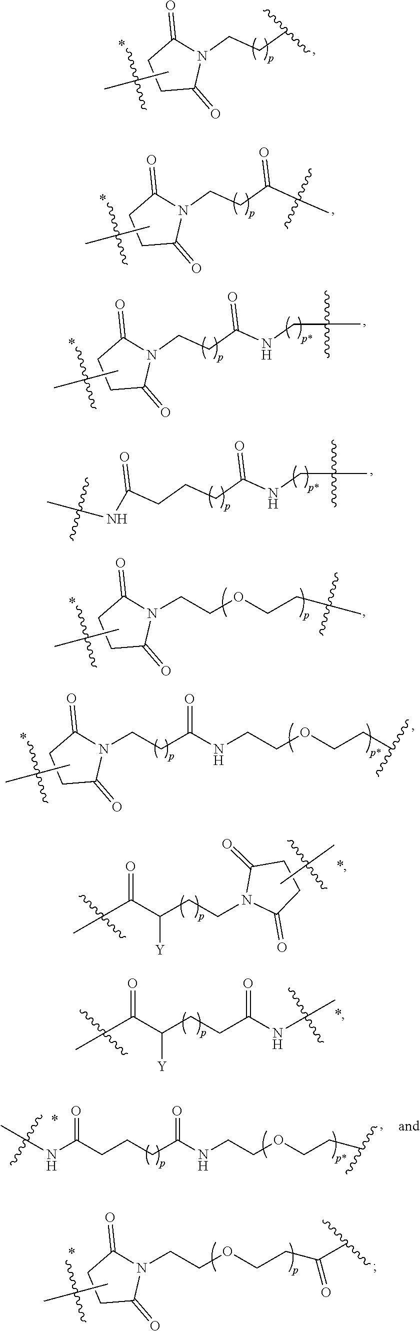

wherein:
   p is an integer from 1 to 10;
   p* is an integer from 1 to 10;
   Y is selected from hydrogen, and C₁-C₆alkyl;
   [symbol] is the point of attachment to the payload; and
   [symbol] is the point of attachment to the binding moiety.

In some aspects, L is selected from

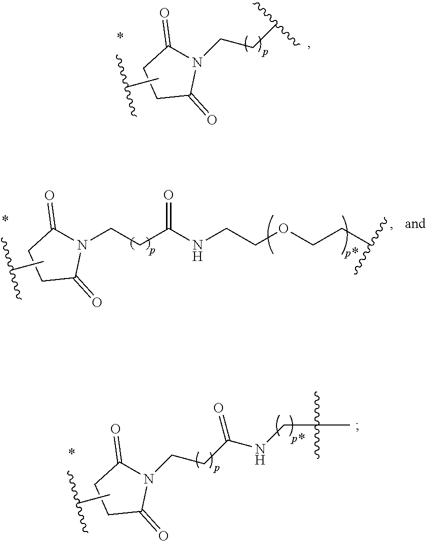

wherein:
   p is an integer from 1 to 10;
   p* is an integer from 1 to 10;
   [symbol] is the point of attachment to the payload; and
   [symbol] the point of attachment to the binding moiety.

In some aspects, L is a cleavable linker. In some aspects, the cleavable linker is cleavable by a protease. In certain aspects, L is selected from

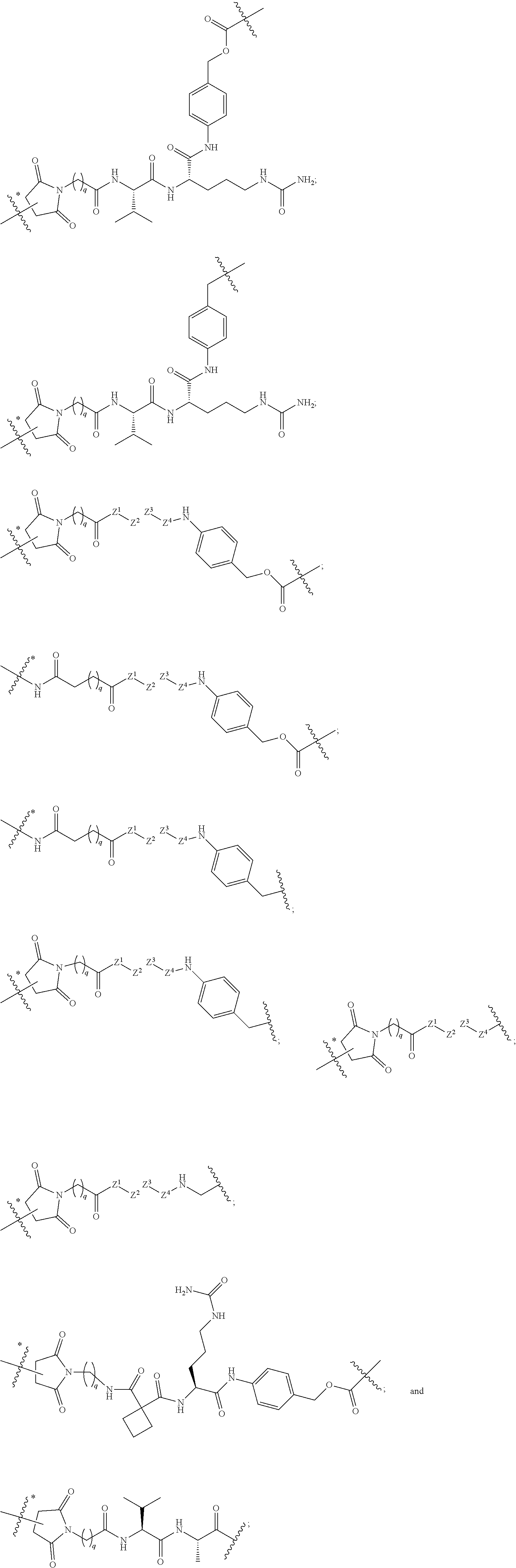

wherein:
   q is an integer from 2 to 10;
   Z¹, Z², Z³, and Z⁴ are each independently absent or a naturally-occurring amino acid residue in the L- or D-configuration, provided that at least two of Z¹, Z², Z³, and Z⁴ are amino acid residues;
   [symbol] is the point of attachment to the payload; and
   [symbol] is the point of attachment to the binding moiety.

In some aspects, Z¹, Z², Z³, and Z⁴ are independently absent or selected from the group consisting of L-valine, D-valine, L-citrulline, D-citrulline, L-alanine, D-alanine, L-glutamine, D-glutaimine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, and Z⁴ are amino acid residues.

In some aspects:
Z¹ is absent or glycine;
Z² is absent or selected from the group consisting of L-glutamine, D-glutamine, L-glutamic acid, D-glutamic acid, L-aspartic acid, D-aspartic acid, L-alanine, D-alanine, and glycine;
Z³ is selected from the group consisting of L-valine, D-valine, L-alanine, D-alanine, L-phenylalanine, D-phenylalanine, and glycine; and
Z⁴ is selected from the group consisting of L-alanine, D-alanine, L-citrulline, D-citrulline, L-asparagine, D-asparagine, L-lysine, D-lysine, L-phenylalamine, D-phenylalanine, and glycine.

In certain aspects, L is selected from

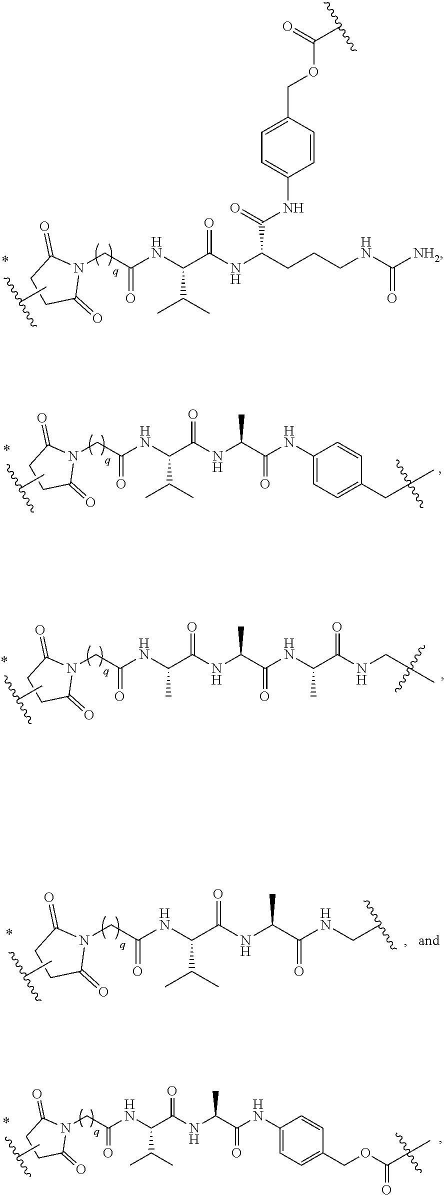

wherein:
   q is an integer from 2 to 10;
   [symbol] is the point of attachment to the payload; and
   [symbol] is the point of attachment to the binding moiety.

In certain aspects, L is a bioreducible linker. In some aspects, L is selected from

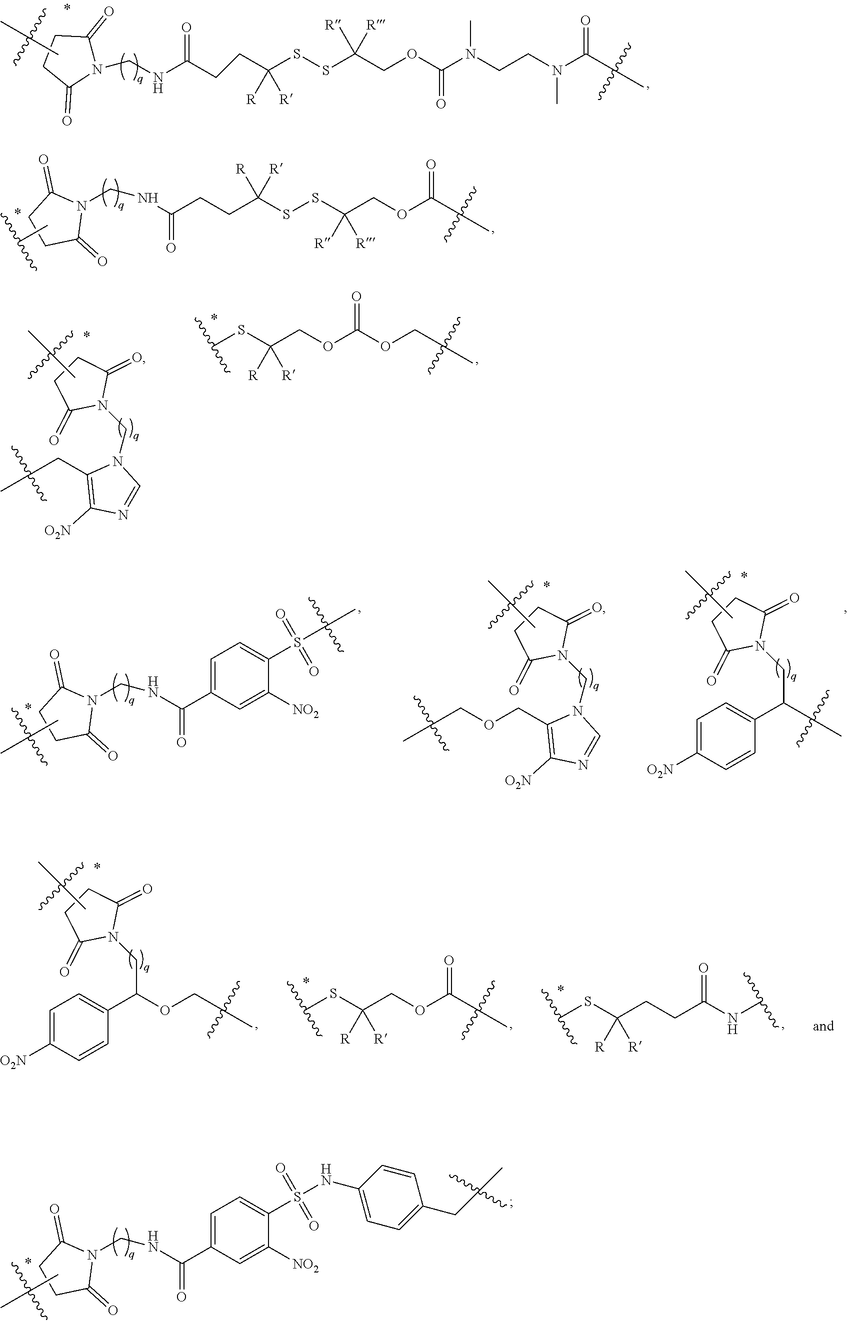

wherein:
   q is an integer from 2 to 10;
   R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring;
   [symbol] is the point of attachment to the payload; and
   [symbol] is the point of attachment to the binding moiety.

In some aspects, L is selected from

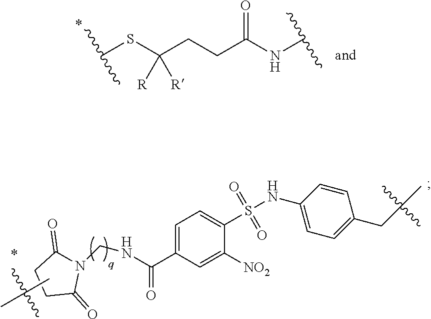

wherein:
   q is an integer from 2 to 10;
   R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring;
   [symbol] is the point of attachment to the payload; and
   [symbol] is the point of attachment to the binding moiety.

In certain aspects, L is an acid cleavable linker.

In some aspects, L is selected from the group consisting of

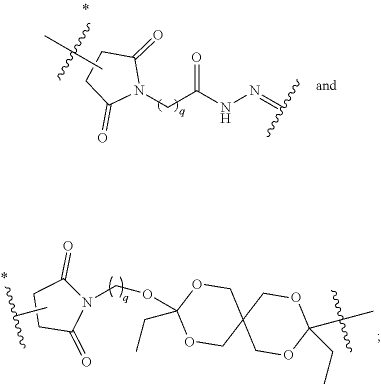

wherein:
   q is an integer from 2 to 10;
   [symbol] is the point of attachment to the payload; and
   [symbol] is the point of attachment to the binding moiety.

In some aspects, L is a click-to-release linker. In some aspects, L is selected from

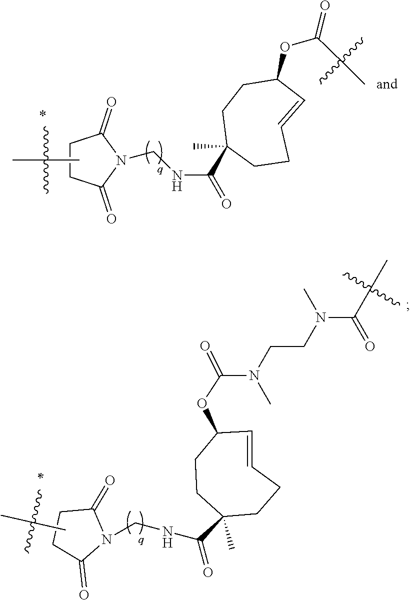

wherein:
   q is an integer from 2 to 10;
   [symbol] is the point of attachment to the payload; and
   [symbol] is the point of attachment to the binding moiety.

In some aspects, L is a pyrophosphatase cleavable linker. In some aspects, L is

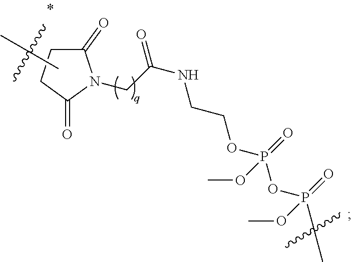

wherein:
   q is an integer from 2 to 10;
   [symbol] is the point of attachment to the payload; and
   [symbol] is the point of attachment to the binding moiety.

In some aspects, L is a beta-glucuronidase cleavable linker. In some aspects, L is selected from

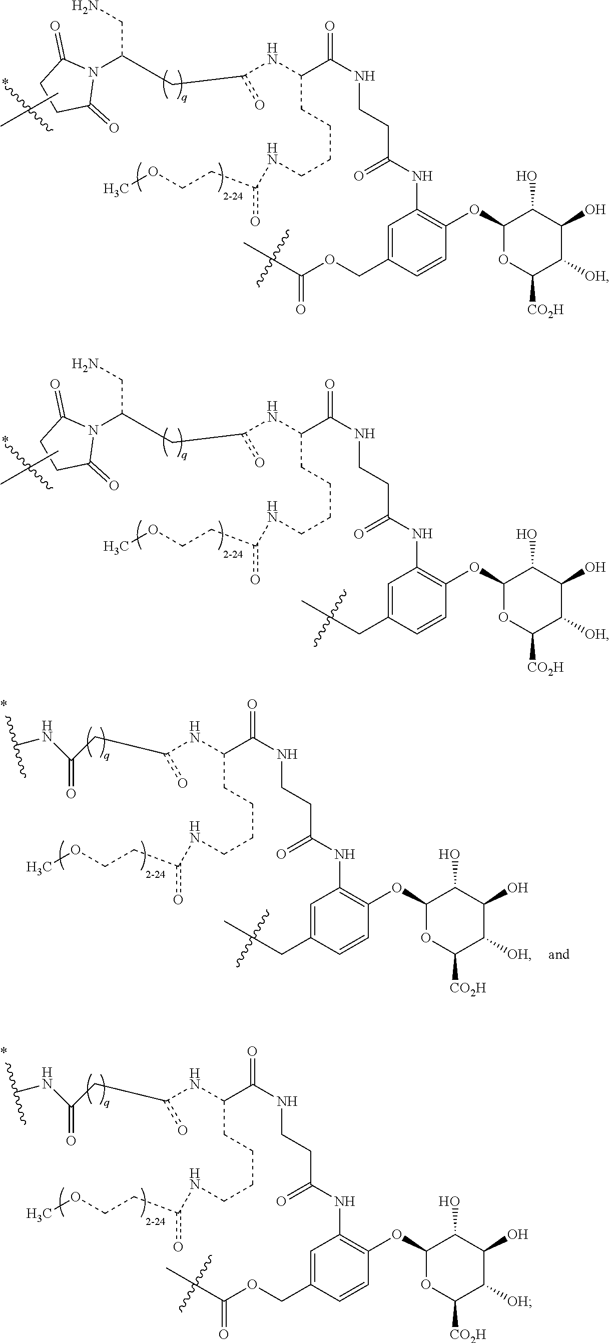

wherein:
   q is an integer from 2 to 10;
   [symbol] is absent or a bond;
   [symbol] is the point of attachment to the payload; and
   [symbol] is the point of attachment to the binding moiety.

In some aspects, L is

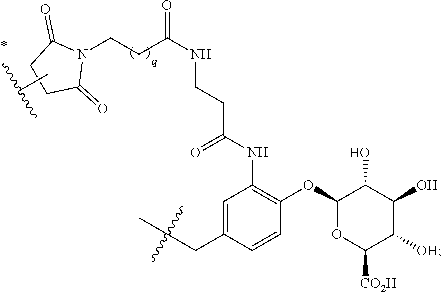

wherein:
   q is an integer from 2 to 10;
   [symbol] is absent or a bond;
   [symbol] is the point of attachment to the payload; and
   [symbol] is the point of attachment to the binding moiety.

In some aspects, the present disclosure provides a conjugate selected from:

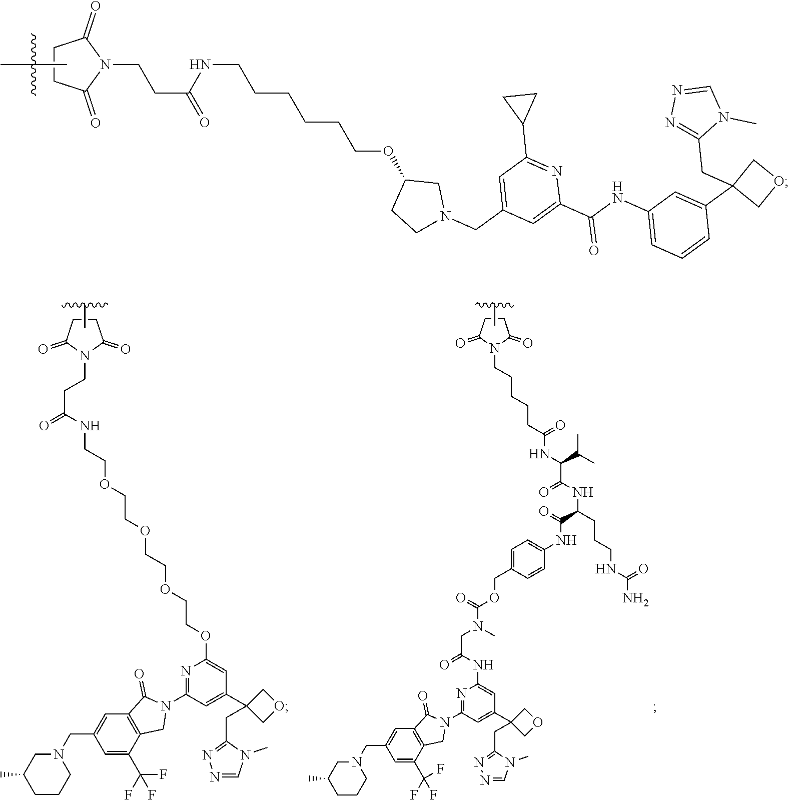

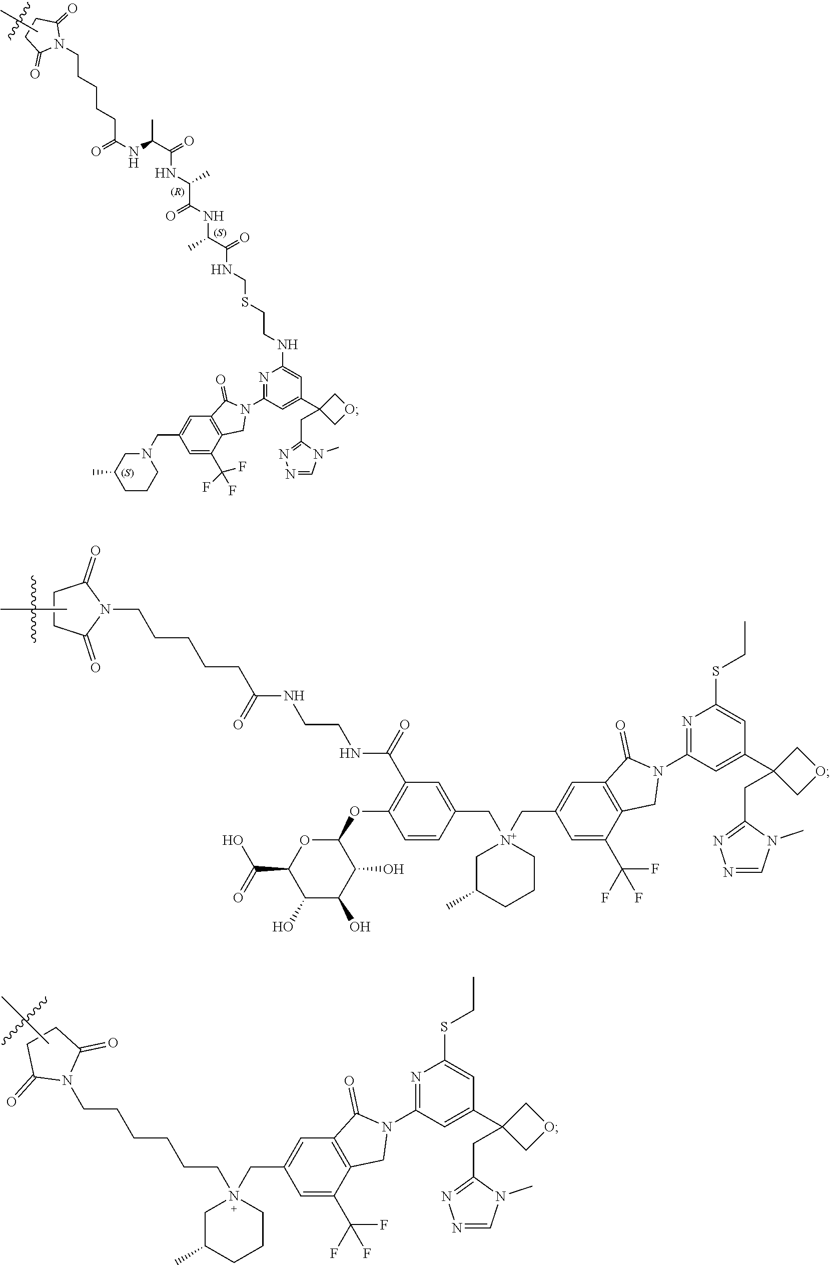

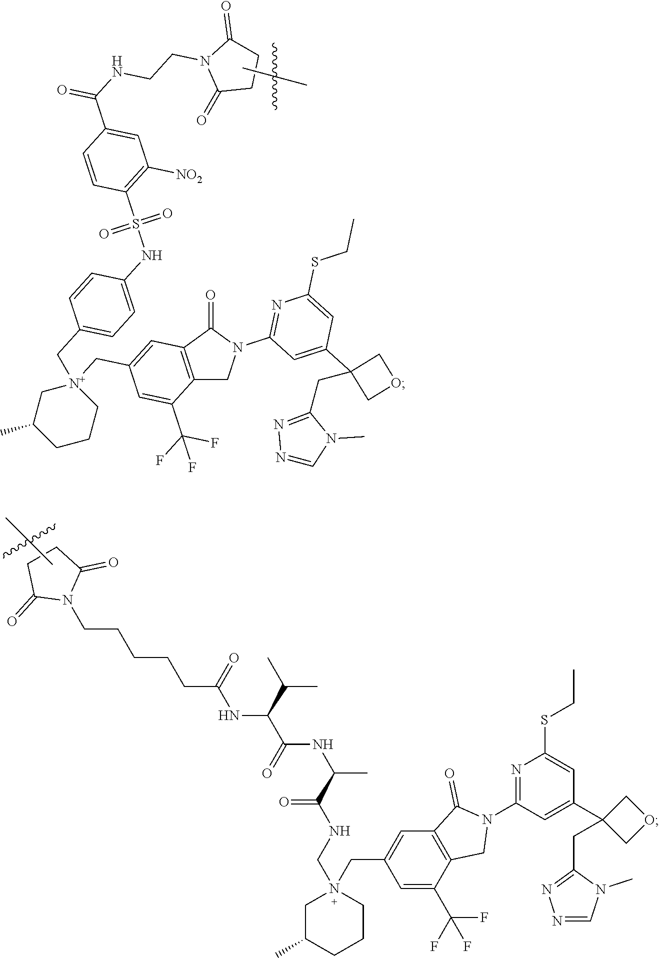

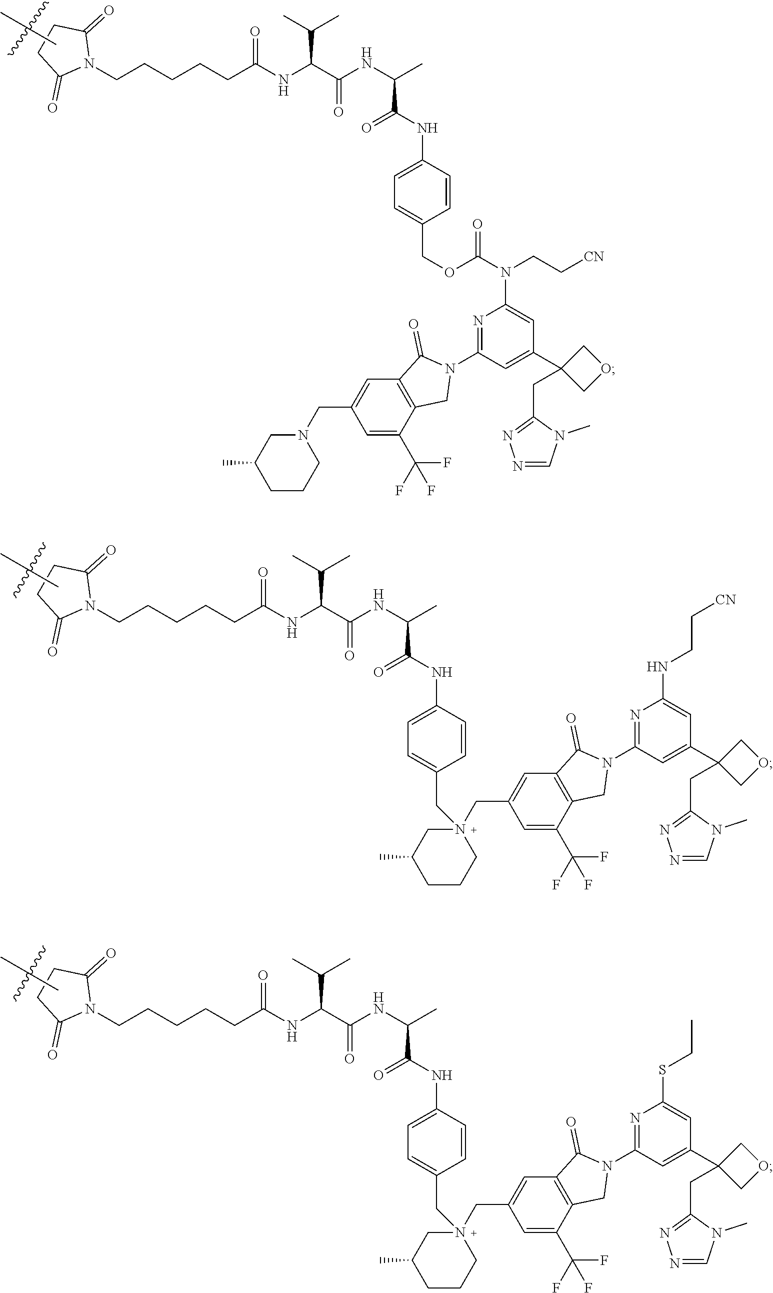

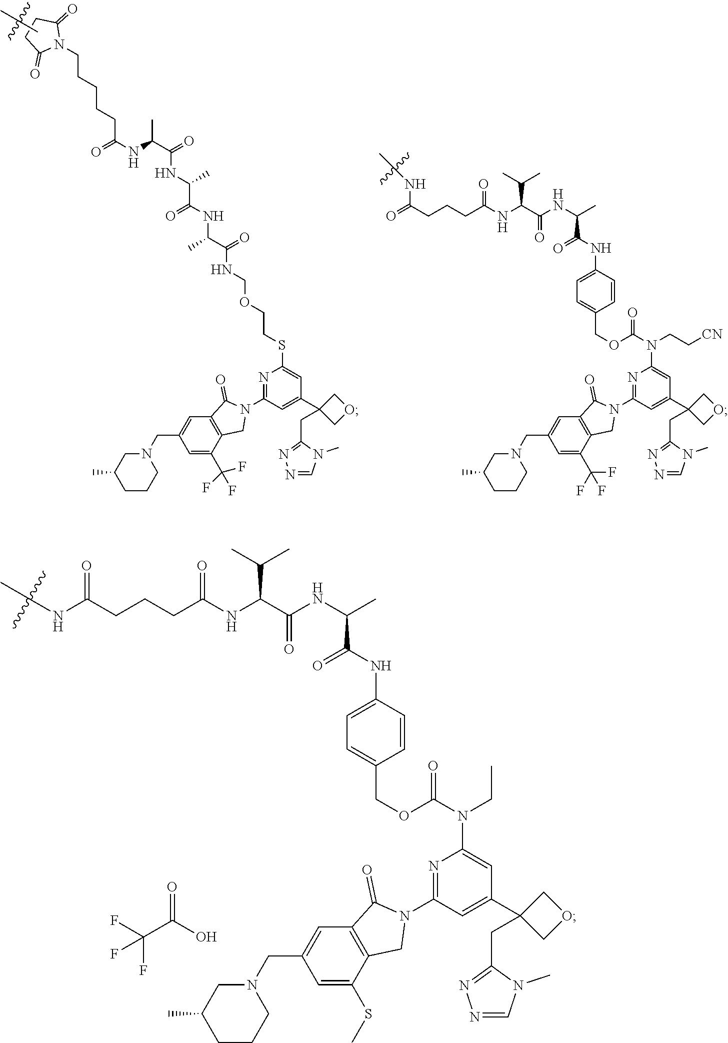

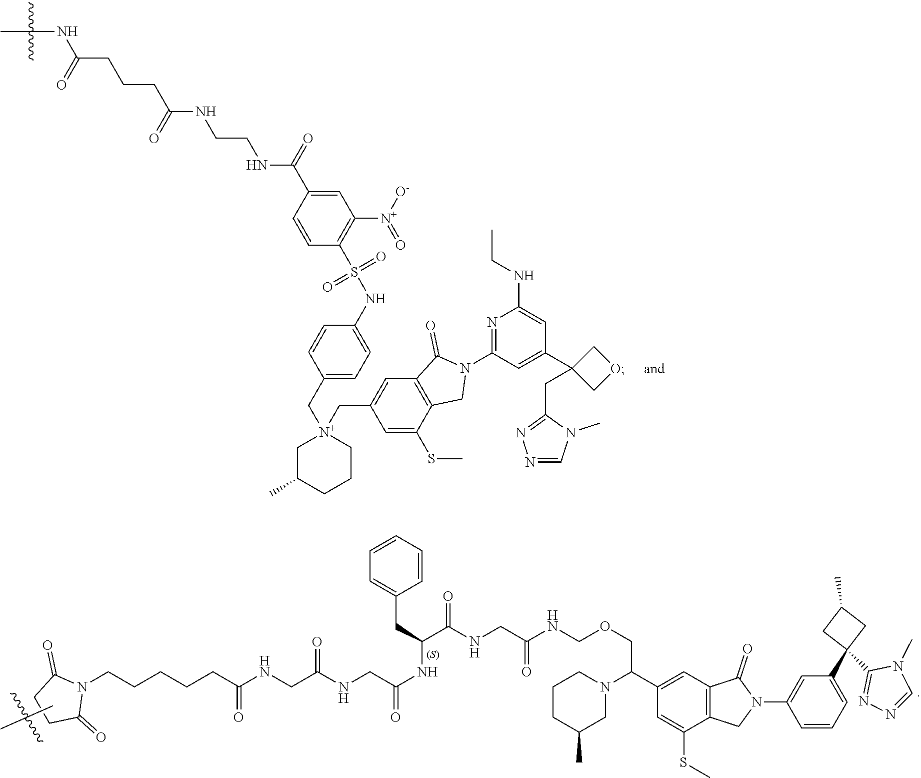

In some aspects, the linker of the conjugate is attached to a cysteine, lysine, tyrosine, or glutamine in the Bm. In some aspects, the cysteine or lysine is an engineered cysteine or lysine. In some aspects, the cysteine or lysine is endogenous to the Bm.

In some aspects, the binding moiety is an antibody or antigen-binding fragment thereof.

In certain aspects, the present disclosure provides conjugates wherein L is attached to an engineered cysteine at heavy chain position S239 and/or K334 of the antibody or antigen binding portion thereof according to EU numbering.

In some aspects, L is attached to a glutamine at heavy chain position 295 of the antibody or antigen binding portion thereof according to EU numbering.

In certain aspects, the binding moiety is an antibody or antigen-binding fragment thereof comprising CDR sequences of amino acids 31-35, 50-66, and 99-109 of SEQ ID NO:10 and amino acids 24-38, 54-60, and 93-101 of SEQ ID NO:11.

In some aspects, the binding moiety is an antibody or antigen-binding fragment thereof comprising a variable heavy chain comprising the amino acid sequence of SEQ ID NO:10 and a variable light chain comprising the amino acid sequence of SEQ ID NO:11.

In some aspects, the binding moiety is an antibody or antigen-binding fragment thereof comprising a heavy chain comprising the amino acid sequence of SEQ ID NO:26 and a light chain comprising the amino acid sequence of SEQ ID NO:27.

In some aspects, the binding moiety is an antibody or antigen-binding fragment thereof comprising the heavy and light chain CDR sequences of amino acids 31-35, 50-66, and 99-102 of SEQ ID NO:12 and amino acids 24-34, 50-56, and 89-97 of SEQ ID NO:13.

In some aspects, the binding moiety is an antibody or antigen-binding fragment thereof comprising a variable heavy chain comprising the amino acid sequence of SEQ ID NO:12 and a variable light chain comprising the amino acid sequence of SEQ ID NO:13.

In some aspects, the binding moiety is capable of specifically binding to CD25.

In some aspects, the binding moiety is an antibody or antigen-binding fragment thereof comprising CDR sequences of amino acids 31-35, 50-66, and 99-111 of SEQ ID NO: 4 and amino acids 24-34, 50-56, and 89-97 of SEQ ID NO:5.

In some aspects, the binding moiety is an antibody or antigen-binding fragment thereof comprising a variable heavy chain comprising the amino acid sequence of SEQ ID NO:4 and a variable light chain comprising the amino acid sequence of SEQ ID NO:5.

In some aspects, the binding moiety is an antibody or antigen-binding fragment thereof comprising CDR sequences of amino acids 31-35, 50-65, and 98-108 of SEQ ID NO: 6 and amino acids 24-33, 49-55, and 88-96 of SEQ ID NO:7.

In some aspects, the binding moiety is an antibody or antigen-binding fragment thereof comprising a variable heavy chain comprising the amino acid sequence of SEQ ID NO:6 and a variable light chain comprising the amino acid sequence of SEQ ID NO:7.

In some aspects, the binding moiety is an antibody or antigen or antigen-binding fragment thereof comprising CDR sequences of amino acids 31-35, 50-65, or 98-108 of SEQ ID NO:8 and amino acids 24-33, 49-55, and 88-96, of SEQ ID NO:9.

In some aspects, the binding moiety is an antibody or antigen-binding fragment thereof comprising a variable heavy chain comprising the amino acid sequence of SEQ ID NO:8 and a variable light chain comprising the amino acid sequence of SEQ ID NO:9.

In some aspects, the binding moiety is capable of specifically binding to CD7.

In some aspects, the binding moiety is an antibody or antigen-binding fragment thereof comprising CDR sequences of amino acids 31-35, 50-66, and 99-112 of SEQ ID NO:14 and amino acids 23-36, 52-58, and 91-99 of SEQ ID NO:15.

In some aspects, the binding moiety is an antibody or antigen-binding fragment thereof comprising a variable heavy chain comprising the amino acid sequence of SEQ ID NO:14 and a variable light chain comprising the amino acid sequence of SEQ ID NO:15.

In some aspects, the antibody or antigen-binding fragment thereof comprises a heavy chain constant region.

In some aspects, the heavy chain constant region comprises an Fc silent mutation.

In some aspects, the heavy chain constant region is an IgG heavy chain constant region.

In some aspects, the IgG heavy chain constant region is an IgG1 heavy chain constant region.

In some aspects, the heavy chain constant region comprises the amino acid sequence of SEQ ID NO:20.

In some aspects, the IgG heavy chain constant region is an IgG4 heavy chain constant region.

In some aspects, the heavy chain constant region comprises the amino acid sequence of SEQ ID NO:21.

In some aspects, the present disclosure provides a conjugate as described herein, or a pharmaceutically acceptable salt thereof, wherein (a) the binding moiety is pembrolizumab or (b) the binding moiety is Nivolumab.

In some aspects, the present disclosure provides a conjugate as described herein, or a pharmaceutically acceptable salt thereof, wherein (a) the binding moiety is a686, (b) the binding moiety is MA251, or (c) the binding moiety is humanized MA251.

In some aspects, the binding moiety is a small molecule.

In some aspects, the present disclosure provides a conjugate as described herein wherein a is 1 to 40. In some aspects, a is 1 to 10. In some aspects, a is 2 to 8.

In some aspects, the present disclosure provides a conjugate as described herein that is capable of increasing effector T cell activity.

In some aspects, the present disclosure provides a conjugate as described herein that is capable of increasing effector T cell proliferation.

In some aspects, the present disclosure provides a conjugate as described herein that is capable of increasing migration of an effector T cell to a tumor cell.

In some aspects, the present disclosure provides a conjugate as described herein that is capable of reducing effector T cell exhaustion.

In some aspects, the present disclosure provides a conjugate as described herein that increases IFN-y secretion from T cells over background by about 2.5 to about 3fold; and/or increases IL-2 secretion from T cells over background by about 6 to about 8fold.

In some aspects, the present disclosure provides a composition comprising a compound as described herein, or a pharmaceutically acceptable salt thereof.

In some aspects, the present disclosure provides a composition comprising a conjugate as described herein, or a pharmaceutically acceptable salt thereof.

In some aspect, the present disclosure provides a method of treating cancer in a subject in need thereof, the method comprising administering to the subject a compound described herein, a conjugate described herein, or a composition described herein. In some aspects, the cancer is (i) resistant or refractory to an anti-PD1 therapy, optionally wherein the anti-PD1 therapy is nivolumab, pembrolizumab, and/or cemiplimab and/or (ii) resistant or refractory to an anti-PDL1 therapy, optionally wherein the anti-PDL1 therapy is durvalumab, atezolizumab, and/or avelumab.

In some aspects, the present disclosure provides a method of treating a condition that would benefit from an increased immune response in a subject in need thereof, the method comprising administering to the subject a compound described herein, a conjugate described herein, or a composition described herein. In some aspects, the condition is an infection, optionally wherein the infection is a viral infection, a bacterial infection, or a parasitic infection, an immunosuppressive disease or disorder, or multiple sclerosis.

In some aspects, the present disclosure provides a method of increasing the activity of an immune cell comprising contacting the immune cell with a compound described herein, a conjugate described herein, or a composition described herein.

In some aspects, the present disclosure provides a method of increasing proliferation of an immune cell comprising contacting an immune cell with a compound described herein, a conjugate described herein, or a composition described herein.

In some aspects, the present disclosure provides a method of increasing migration of an immune cell to a tumor cell comprising contacting an immune cell with a compound described herein, a conjugate described herein, or a composition described herein.

In some aspects, the present disclosure provides method of reducing exhaustion of an immune cell comprising contacting an immune cell with a compound described herein, a conjugate described herein, or a composition described herein.

In some aspects, the present disclosure provides a method of increasing secretion of IFN-γ or IL-2 from an immune cell comprising contacting the immune cell with a compound described herein, a conjugate described herein, or a composition described herein. In some aspects, the immune cell is T cell, optionally wherein the T cell is an effector T cell. In some aspects, the immune cell is a natural killer (NK) cell.

In some aspects, the present disclosure provides a method of enhancing and/or sustaining an antigen recall response of a T comprising contacting the T cell with a compound described herein, a conjugate described herein, or a composition described herein.

In some aspects, the present disclosure provides a method of delivering a payload that is capable of activating an effector T cell to an immune cell, the method comprising contacting an effector T cell with a conjugate described herein, or a composition comprising a conjugate described herein. In some aspects, the contacting is in vitro. In some aspects, the contacting is in a subject, optionally wherein the subject has cancer or a condition that would benefit from an increased immune response.

### BRIEF DESCRIPTION OF THE FIGURES

FIG. 1 is a histogram showing the protein-ligand interaction fractions between Cbl-b and Compound 147 and between Cbl-b and Compound 118.

FIG. 2 is graphs showing the activity of conjugates in a mixed lymphocyte reaction.

FIG. 3A is a graph showing the activity of conjugates in a mixed lymphocyte reaction in the presence and absence of TGF-β.

FIG. 3B is a graph showing the activity of conjugates in a mixed lymphocyte reaction in the presence and absence of Treg cells.

FIG. 4 is a graph showing in vitro tumor infiltration lymphocyte (TIL) activation.

FIGS. 5 and 6 are graphs showing the in vivo activity of a conjugate against B16F10 melanoma cells in mice as compared to an unconjugated antibody and vehicle control.

FIG. 7 is a graph showing activation of exhausted T-cells by anti-PD-1-Cbl-B inhibitor conjugates.

FIG. 8 is a graph showing antigen recall responses (as measured by levels of IFN-7) of T cells treated with anti-PD-1-Cbl-B inhibitor conjugates.

### DETAILED DESCRIPTION

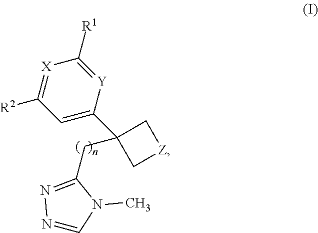

or a pharmaceutically acceptable salt thereof, wherein:
   n is 0, 1, or 2;
   X and Y are each independently CH or N;
   Z is selected from CH(CH₃), O, and SO₂; or
   Z is selected from CH(CH₃), NH, N(CH₃), O, and SO₂;
   R¹ is selected from hydrogen, —CN, —NHR^{z}, —R^{a}, —NR^{a}R^{b}, —OR^{a}, —NHC(O)R^{a}, —NHC(S)R^{a}, —NHC(O)NHR^{a}, —NHC(S)NHR^{a}, —SR^{a}, C₃-C₆cycloalkyl, and a 3- to 6-membered heterocyclyl ring; wherein:
      R^{z} is selected from

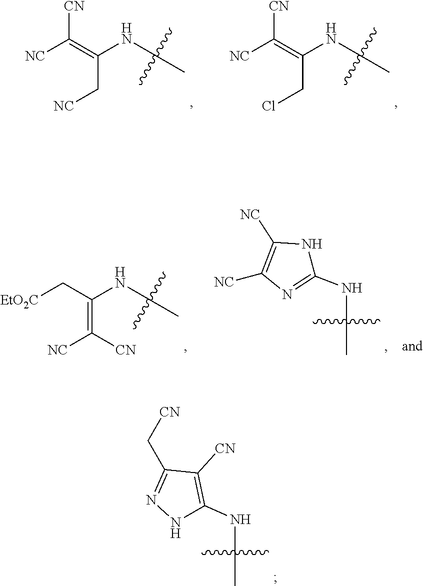 [SMILES: *NC(CC#N)=C(C#N)C#N ; *NC(CCl)=C(C#N)C#N ; *NC(CC(=O)OCC)=C(C#N)C#N ; *Nc1nc(C#N)c(C#N)[nH]1 ; *Nc1[nH]nc(CC#N)c1C#N]

R^{a} and R^{b} are independently selected from hydrogen, C₂-C₆alkenyl, C₁-C₆alkyl, amido(C₁-C₆alkyl), amino(C₁-C₆alkyl), azido(C₁-C₆alkyl), C₂-C₆alkynyl, carboxy(C₁-C₆alkyl), cyano(C₁-C₆alkyl), C₃-C₆cycloalkyl optionally substituted with a cyano group, dimethylamino(C₁-C₆alkyl), a 3- to 6-membered heterocyclyl ring, 3-6-membered heterocyclyl(C₁-C₃alkyl), hydroxy(C₁-C₆alkyl), methoxy(C₁-C₆alkyl), methylamino(C₁-C₆alkyl), NR^{c}R^{d}(C₁-C₆alkyl), HS(C₁-C₆alkyl), and CH₃S(C₁-C₆alkyl), wherein R^{c} and R^{d} are independently selected from hydrogen, C₂alkenylcarbonyl, and methyl; or,
R^{a} and R^{b}, together with the nitrogen atom to which they are attached, form a five- or six-membered ring optionally containing one additional nitrogen atom, wherein the ring is optionally substituted with one group selected from R^{a}, —C(O)R^{a}, —SO₂R^{a}, azido, and cyano;
wherein each C₃-C₆cycloalkyl, each 3- to 6-membered heterocyclyl ring, and the heterocyclyl part of the 3- to 6-membered heterocyclyl(C₁-C₃alkyl) ring are optionally substituted with one, two, or three groups independently selected from C₁-C₃alkyl, C₂alkynyl, amido, azido, carboxy, cyano, dimethylamino, hydroxy, methoxy, methylamino, HS—, and CH₃S—; and
R² is selected from

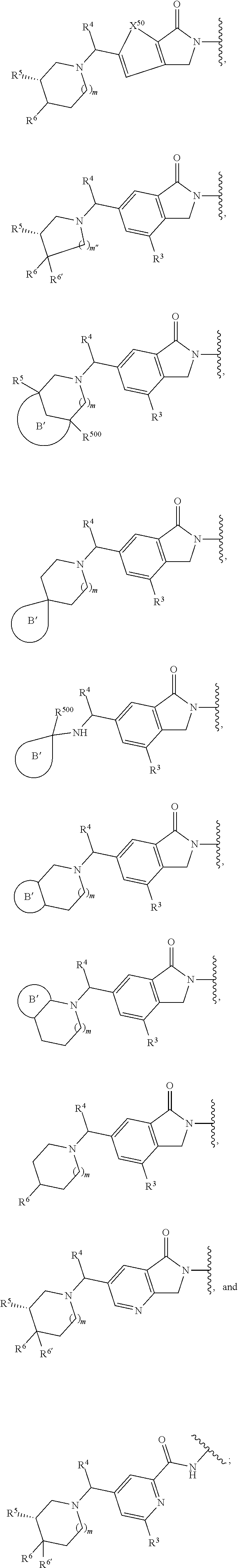

wherein
m is 0, 1, 2, or 3;
m″ is 0, 1, 2, 3, or 4;
B′ is a 3-7 membered saturated or unsaturated ring optionally containing one or two heteroatoms independently selected from nitrogen, oxygen, and sulfur; wherein the ring is optionally substituted with one or two substituents independently selected from —OH, —CH₂SH, CH₂SCH₃, —CH₂OH, —CH₂NH₂, and —CH₂NHCH₃;
each R⁵⁰⁰ is independently selected from hydrogen, C₁-C₆alkyl, halo, —OH, and —CH₂OH; and
X⁵⁰ is selected from O, NH, NCH₃, and S;
R³ is selected from hydrogen, acetyl, amino, C₁-C₆alkylamino, C₁-C₆alkylaminomethyl, C₁-C₆alkylcarbonyl, aminoC₁-C₆alkyl, aminocarbonyl, aminomethyl, carboxy, cyano, C₃cycloalkyl, formyl, hydroxy, hydroxyC₁-C₆alkyl, methoxy, oxazolyl, —SH, —SCH₃, —SOCH₃, —SO₂CH₃, —SO(═NH)CH₃, tetrazolyl, thiazolyl, and trifluoromethyl, wherein the C₃cycloalkyl is optionally substituted with a hydroxy group;
R⁴ is selected from hydrogen, methyl, —CH₂OH, —CH₂SH, and —CH₂SCH₃;
R⁵ is selected from hydrogen, hydroxy, —CH₂SH, —CH₂SCH₃, and methyl;
optionally provided that when R⁵ is hydroxy or methyl, and R⁴ is hydrogen, then R¹ is other than C₃-C₆cycloalkyl, a 3- to 6-membered heterocyclyl ring, hydroxy, hydroxy(C₁-C₆alkyl), —OR^{a} wherein R^{a} is C₁-C₆alkyl, a 3- to 6-membered heterocyclyl ring, or hydroxy(C₁-C₆alkyl); or —NR^{a}R^{b}, wherein R^{a} and R^{b} are independently selected from the group consisting of hydrogen, C₁-C₆alkyl, C₃-C₆cycloalkyl, hydroxy(C₁-C₆alkyl), a 3- to 6-membered heterocyclyl ring, or wherein R^{a} and R^{b}, together with the nitrogen atom to which they are attached, form a five- or six-membered ring optionally containing one additional nitrogen atom, wherein the ring is optionally substituted with one group selected from C₁-C₆alkyl or hydroxy(C₁-C₆alkyl); and
R⁶ and R^{6′} are independently selected from hydrogen, cyclopropyl, —CH₂OH, —CH₂SH, —CH₂SCH₃, and —CH₂R²⁰⁰, wherein R²⁰⁰ is a 3-7 membered saturated or unsaturated ring optionally containing one or two heteroatoms independently selected from nitrogen, oxygen, and sulfur.

In some aspects, R⁴ is in the “R” configuration. In some aspects, R⁴ is in the “S” configuration.

In certain aspects, the present disclosure is directed to Cbl-b inhibitors of formula (IA-1):

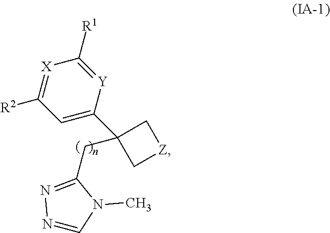

or a pharmaceutically acceptable salt thereof, wherein:
   n is 0, 1, or 2;
   X and Y are each independently CH or N;
   Z is selected from CH(CH₃), O, and SO₂;
   R¹ is selected from hydrogen, —CN, —NHR^{z}, —R^{a}, —NR^{a}R^{b}, —OR^{a}, —NHC(O)R^{a}, —NHC(S)R^{a}, —NHC(O)NHR^{a}, —NHC(S)NHR^{a}, —SR^{a}, C₃-C₆cycloalkyl, and a 3- to 6-membered heterocyclyl ring; wherein:
   R^{z} is selected from

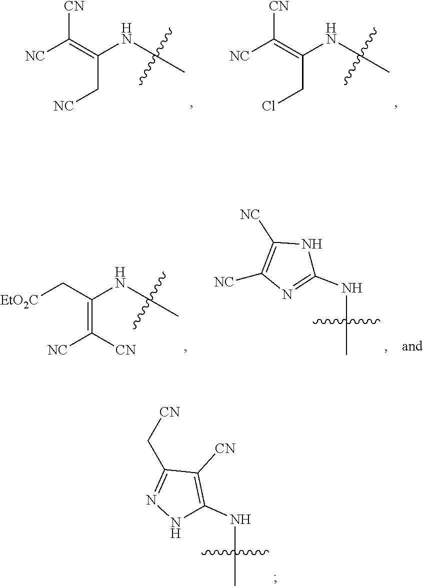 [SMILES: *NC(CC#N)=C(C#N)C#N ; *NC(CCl)=C(C#N)C#N ; *NC(CC(=O)OCC)=C(C#N)C#N ; *Nc1nc(C#N)c(C#N)[nH]1 ; *Nc1[nH]nc(CC#N)c1C#N]

R^{a} and R^{b} are independently selected from hydrogen, C₂-C₆alkenyl, C₁-C₆alkyl, amido(C₁-C₆alkyl), amino(C₁-C₆alkyl), azido(C₁-C₆alkyl), C₂-C₆alkynyl, carboxy(C₁-C₆alkyl), cyano(C₁-C₆alkyl), C₃-C₆cycloalkyl, dimethylamino(C₁-C₆alkyl), a 3-6-membered heterocyclyl ring, 3-6-membered heterocyclyl(C₁-C₃)alkyl, hydroxy(C₁-C₆alkyl), methoxy(C₁-C₆alkyl), methylamino(C₁-C₆alkyl), NR^{c}R^{d}(C₁-C₆alkyl), HS(C₁-C₆alkyl), and CH₃S(C₁-C₆alkyl), wherein R^{c} and R^{d} are independently selected from hydrogen, C₂alkenylcarbonyl, and methyl; or,
R^{a} and R^{b}, together with the nitrogen atom to which they are attached, form a five- or six-membered ring optionally containing one additional nitrogen atom, wherein the ring is optionally substituted with one group selected from R^{a}, —C(O)R^{a}, —SO₂R^{a}, azido, and cyano;
wherein each C₃-C₆cycloalkyl, each 3-6-membered heterocyclyl ring, and the heterocyclyl part of the 3-6 membered heterocyclyl(C₁-C₃alkyl) are optionally substituted with one, two, or three groups independently selected from C₁-C₃alkyl, C₂alkynyl, amido, azido, carboxy, cyano, dimethylamino, hydroxy, methoxy, methylamino, HS—, and CH₃S—; and
R² is

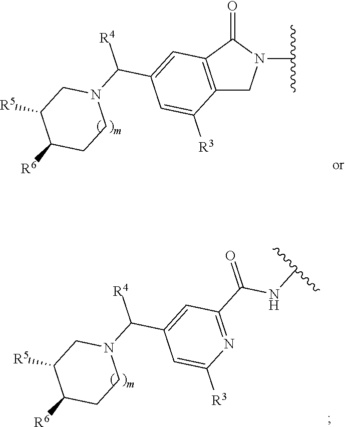

wherein
m is 0, 1, 2, or 3;
R³ is selected from hydrogen, acetyl, amino, C₁-C₆alkylamino, C₁-C₆alkylaminomethyl, aminoC₁-C₆alkyl, aminocarbonyl, aminomethyl, carboxy, cyano, C₃cycloalkyl, formyl, hydroxy, hydroxyC₁-C₆alkyl, methoxy, oxazolyl, —SH, —SCH₃, tetrazolyl, thiazolyl, and trifluoromethyl, wherein the C₃ cycloalkyl is optionally substituted with a hydroxy group;
R⁴ and R⁶ are independently selected from hydrogen, —CH₂SH, and —CH₂SCH₃; and
R⁵ is selected from hydroxy, —CH₂SH, —CH₂SCH₃, and methyl.

In some aspects, R⁴ is in the “R” configuration. In some aspects, R⁴ is in the “S” configuration.

The present disclosure also provides conjugates that activate effector T cells, and further provides methods of treating cancer, methods of increasing T cell activity, methods of increasing T cell proliferation, methods of increasing migration of a T cell to a tumor cell, methods of reducing T cell exhaustion, and methods of delivering a payload capable of activating an effector T cell using the conjugates.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The terms “effector T cell,” “T effector cell,” “Teff,” and “T-eff” are used interchangeably. In some aspects, the “effector T cell” is a CD8+ cytotoxic T cell.

An “activator” of an effector T cell refers to a molecule that is capable of promoting one or more functions of an effector T cell. An activator of an effector T cell can promote effector T cell function, for example, by promoting proliferation, survival, and/or migration of an effector T cell, by promoting production of effector cytokines and/or cytotoxic mediators from an effector T cell, and/or by reducing exhaustion of an effector T cell. An “activator” of an effector T cell can also be capable of activating one or more other cell types, e.g., one or more other T cell types, so long as it is capable of activating an effector T cell. As used herein, the term “interleukin-2 receptor subunit alpha,” “IL2-RA,” “IL2RA,” “CD-25,” or “CD25” refers to mammalian CD25 polypeptides including, but not limited to, native CD25 polypeptides and isoforms of CD25 polypeptides. These terms encompass full-length, unprocessed CD25 polypeptides as well as forms of CD25 polypeptides that result from processing within the cell.

As used herein, the term “human CD25” refers to a polypeptide comprising amino acids 22-272 of SEQ ID NO:1. SEQ ID NO:1 provides the sequence of mature human CD25 (amino acids 22-272) as well as its signal sequence (amino acids 1-21, underlined below).

(SEQ ID NO: 1)
MDSYLLMWGLLTFIMVPGCQAELCDDDPPEIPHATFKAMAYKEGTMLNC
ECKRGFRRIKSGSLYMLCTGNSSHSSWDNQCQCTSSATRNTTKQVTPQP
EEQKERKTTEMQSPMQPVDQASLPGHCREPPPWENEATERIYHFVVGQM
VYYQCVQGYRALHRGPAESVCKMTHGKTRWTQPQLICTGEMETSQFPGE
EKPQASPEGRPESETSCLVTTTDFQIQTEMAATMETSIFTTEYQVAVAG
CVFLLISVLLLSGLTWQRRQRKSRRTI

A “CD25 polynucleotide,” “CD25 nucleotide,” or “CD25 nucleic acid” refers to a polynucleotide encoding a CD25 polypeptide.

As used herein, the term “programmed cell death protein 1,” “PD-1,” or “PD1” refers to mammalian PD1 polypeptides including, but not limited to, native PD1 polypeptides and isoforms of PD1 polypeptides. These terms encompass full-length, unprocessed PD1 polypeptides as well as forms of PD1 polypeptides that result from processing within the cell.

As used herein, the term “human PD1” refers to a polypeptide comprising amino acids 24-288 of SEQ ID NO:2. SEQ ID NO:2 provides the sequence of mature human PD1 (amino acids 24-288) as well as its signal sequence (amino acids 1-23, underlined below).

(SEQ ID NO: 2)
MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL.

A “PD1 polynucleotide,” “PD1 nucleotide,” or “PD1 nucleic acid” refers to a polynucleotide encoding a PD1 polypeptide.

As used herein, the term “T cell antigen CD-7,” “CD-7,” or “CD7” refers to mammalian CD7 polypeptides including, but not limited to, native CD7 polypeptides and isoforms of CD7 polypeptides. These terms encompass full-length, unprocessed CD7 polypeptides as well as forms of CD7 polypeptides that result from processing within the cell.

As used herein, the term “human CD7” refers to a polypeptide comprising amino acids 26-240 of SEQ ID NO:3. SEQ ID NO:3 provides the sequence of mature human CD7 (amino acids 26-240) as well as its signal sequence (amino acids 1-25, underlined below).

(SEQ ID NO: 3)
MAGPPRLLLLPLLLALARGLPGALAAQEVQQSPHCTTVPVGASVNITCS
TSGGLRGIYLRQLGPQPQDIIYYEDGVVPTTDRRFRGRIDFSGSQDNLT
ITMHRLQLSDTGTYTCQAITEVNVYGSGTLVLVTEEQSQGWHRCSDAPP
RASALPAPPTGSALPDPQTASALPDPPAASALPAALAVISFLLGLGLGV
ACVLARTQIKKLCSWRDKNSAACVVYEDMSHSRCNTLSSPNQYQ.

A “CD7 polynucleotide,” “CD7 nucleotide,” or “CD7 nucleic acid” refers to a polynucleotide encoding a CD7 polypeptide.

As used herein, the term “Casitas B-lineage lymphoma proto-oncogene b,” “E3 ubiquitin-protein ligase Cbl-b,” or “Cbl-b” refers to mammalian Cbl-b polypeptides including, but not limited to, native Cbl-b polypeptides and isoforms of Cbl-b polypeptides. These terms encompass full-length, unprocessed Cbl-b polypeptides as well as forms of Cbl-b polypeptides that result from processing within the cell.

As used herein, the term “human Cbl-b” refers to a polypeptide comprising the amino acid sequence of SEQ ID NO:25.

(SEQ ID NO: 25)
MANSMNGRNPGGRGGNPRKGRILGIIDAIQDAVGPPKQAAADRRTVEKT
WKLMDKVVRLCQNPKLQLKNSPPYILDILPDTYQHLRLILSKYDDNQKL
AQLSENEYFKIYIDSLMKKSKRAIRLFKEGKERMYEEQSQDRRNLTKLS
LIFSHMLAEIKAIFPNGQFQGDNFRITKADAAEFWRKFFGDKTIVPWKV
FRQCLHEVHQISSGLEAMALKSTIDLTCNDYISVFEFDIFTRLFQPWGS
ILRNWNFLAVTHPGYMAFLTYDEVKARLQKYSTKPGSYIFRLSCTRLGQ
WAIGYVTGDGNILQTIPHNKPLFQALIDGSREGFYLYPDGRSYNPDLTG
LCEPTPHDHIKVTQEQYELYCEMGSTFQLCKICAENDKDVKIEPCGHLM
CTSCLTAWQESDGQGCPFCRCEIKGTEPIIVDPFDPRDEGSRCCSIIDP
FGMPMLDLDDDDDREESLMMNRLANVRKCTDRQNSPVTSPGSSPLAQRR
KPQPDPLQIPHLSLPPVPPRLDLIQKGIVRSPCGSPTGSPKSSPCMVRK
QDKPLPAPPPPLRDPPPPPPERPPPIPPDNRLSRHIHHVESVPSRDPPM
PLEAWCPRDVFGTNQLVGCRLLGEGSPKPGITASSNVNGRHSRVGSDPV
LMRKHRRHDLPLEGAKVFSNGHLGSEEYDVPPRLSPPPPVTTLLPSIKC
TGPLANSLSEKTRDPVEEDDDEYKIPSSHPVSLNSQPSHCHNVKPPVRS
CDNGHCMLNGTHGPSSEKKSNIPDLSIYLKGDVFDSASDPVPLPPARPP
TRDNPKHGSSLNRTPSDYDLLIPPLGEDAFDALPPSLPPPPPPARHSLI
EHSKPPGSSSRPSSGQDLFLLPSDPFVDLASGQVPLPPARRLPGENVKT
NRTSQDYDQLPSCSDGSQAPARPPKPRPRRTAPEIHHRKPHGPEAALEN
VDAKIAKLMGEGYAFEEVKRALEIAQNNVEVARSILREFAFPPPVSPRL
NL

A “Cbl-b polynucleotide,” “Cbl-b nucleotide,” or “Cbl-b nucleic acid” refers to a polynucleotide encoding a Cbl-b polypeptide.

The term “antibody,” as used herein, refers to an immunoglobulin molecule that immunospecifically binds an antigen of a target of interest (such as a protein, polypeptide, peptide, carbohydrate, polynucleotide, lipid, or combinations of the foregoing) or part thereof. As used herein, the term “antibody” encompasses intact polyclonal antibodies, intact monoclonal antibodies, chimeric antibodies, humanized antibodies, human antibodies, multispecific antibodies, fusion proteins comprising antibodies, and any other modified immunoglobulin molecule so long as the antibodies exhibit the desired biological activity. An antibody disclosed herein can be of any class (e.g., IgG, IgE, IgM, IgD, and IgA) or subclass (isotype) (e.g., IgG1, IgG2, IgG3, IgG4, IgA1 and IgA2). The different classes of immunoglobulins have different and well known subunit structures and three-dimensional configurations. The immunoglobulins can be derived from any species. In one aspect, the immunoglobulin is of human, murine, or rabbit origin. Antibodies can be naked or conjugated to other molecules such as activators of effector T cells including small molecule activators of effector T cells.

An “intact antibody” is one which comprises an antigen-binding variable region as well as a light chain constant domain (CL) and heavy chain constant domains, CH1, CH2 and CH3. The constant domains can be native sequence constant domains (e.g., human native sequence constant domains) or amino acid sequence variant thereof.

“Antibody fragments” comprise a portion of an intact antibody. An “antigen-binding fragment,” “antigen-binding domain,” or “antigen-binding region,” refers to a portion of an intact antibody that binds to an antigen. Examples of antibody fragments include Fab, Fab′, F(ab′)₂, and Fv fragments; diabodies; linear antibodies; fragments produced by a Fab expression library, anti-idiotypic (anti-Id) antibodies, CDR (complementary determining region), and epitope-binding fragments of any of the above which immunospecifically bind to the target of interest, single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody or antigen-binding fragment thereof. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments which have hinge cysteines between them. Other chemical couplings of antibody fragments are also known.

The term “single domain antibody,” also known as a nanobody, is an antibody fragment consisting of a single monomeric variable antibody domain with a molecular weight of from about 12 kDa to about 15 k Da. Single domain antibodies can be based on heavy chain variable domains or light chains. Examples of single domain antibodies include, but are not limited to, V_{H}H fragments and V_{NAR} fragments.

“Single-chain Fv” or “scFv” antibody fragments comprise the VH and VL domains of antibody, wherein these domains are present in a single polypeptide chain. The Fv polypeptide may further comprise a polypeptide linker between the VH and VL domains which enables the scFv to form the desired structure for antigen binding.

The term “diabodies” refers to small antibody fragments with two antigen-binding sites, which fragments comprise a variable heavy domain (VH) connected to a variable light domain (VL) in the same polypeptide chain (VH-VL). By using a linker that is too short to allow pairing between the two domains on the same chain, the domains are forced to pair with the complementary domains of another chain and create two antigen-binding sites.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “monoclonal” antibody or antigen-binding fragment thereof as used herein refers to an antibody or antigen-binding fragment thereof obtained from a population of substantially homogeneous antibodies or antigen-binding fragments thereof, i.e., the individual antibodies or antigen-binding fragments thereof comprising the population are identical except for possible naturally occurring mutations that may be present in minor amounts. Monoclonal antibodies or antigen-binding fragments thereof are highly specific, being directed against a single antigenic site. Furthermore, in contrast to polyclonal antibody preparations which include different antibodies directed against different determinants (epitopes), each monoclonal antibody or antigen-binding fragment thereof is directed against a single determinant on the antigen. In addition to their specificity, the monoclonal antibodies or antigen-binding fragments thereof are advantageous in that they may be synthesized uncontaminated by other antibodies or antigen-binding fragments thereof. The modifier “monoclonal” indicates the character of the antibody or antigen-binding fragment thereof as being obtained from a substantially homogeneous population of antibodies or antigen-binding fragments thereof, and is not to be construed as requiring production of the antibody or antigen-binding fragment thereof by any particular method. For example, the monoclonal antibodies or antigen-binding fragments thereof to be used in accordance with the present disclosure can be made by the hybridoma method, or can be made by recombinant DNA methods. The “monoclonal” antibodies or antigen-binding fragments thereof can also be isolated from phage antibody libraries.

The monoclonal antibodies and antigen-binding fragments thereof herein specifically include “chimeric” antibodies and antigen-binding fragments thereof in which a portion of the heavy and/or light chain is identical with or homologous to corresponding sequences in antibodies or antigen-binding fragments thereof derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies and antigen-binding fragments thereof of interest herein include “primatized” antibodies and antigen-binding fragments thereof comprising variable domain antigen-binding sequences derived from a non-human primate (e.g., Old World Monkey, Ape etc.) and human constant region sequences.

“Humanized” forms of non-human (e.g., rodent) antibodies and antigen-binding fragments thereof are chimeric antibodies and antigen-binding fragments thereof that contain minimal sequence derived from non-human immunoglobulin. Humanization is a method to transfer the murine antigen binding information to a non-immunogenic human antibody acceptor, and has resulted in many therapeutically useful drugs. The method of humanization generally begins by transferring all six murine complementarity determining regions (CDRs) onto a human antibody or antigen-binding fragment framework. These CDR-grafted antibodies or antigen-binding fragments thereof generally do not retain their original affinity for antigen binding, and in fact, affinity is often severely impaired. Besides the CDRs, select non-human antibody framework residues must also be incorporated to maintain proper CDR conformation. The transfer of key mouse framework residues to the human acceptor in order to support the structural conformation of the grafted CDRs has been shown to restore antigen binding and affinity. For the most part, humanized antibodies or antigen-binding fragments thereof are human immunoglobulins or fragments thereof (recipient antibody or fragment thereof) in which residues from a hypervariable region of the recipient are replaced by residues from a hypervariable region of a non-human species (donor antibody or fragment thereof) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies or antigen-binding fragments thereof can comprise residues that are not found in the recipient or donor antibody or fragment thereof. These modifications are made to further refine antibody or antigen-binding fragment thereof performance. In general, the humanized antibody or antigen-binding fragment thereof will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody or antigen-binding fragment thereof optionally also will comprise at least a portion of an immunoglobulin constant region (Fc), typically that of a human immunoglobulin.

Chimeric and humanized antibodies and antigen-binding fragments thereof reduce the likelihood of a Human Anti-Mouse Antibody (HAMA) response by minimizing the nonhuman portions of administered antibodies. Furthermore, chimeric and humanized antibodies and antigen-binding fragments thereof can have the additional benefit of activating secondary human immune responses, such as antibody dependent cellular cytotoxicity (ADCC).

An antibody or antigen-binding fragment thereof can have one or more “effector functions” which refer to those biological activities attributable to the Fc region (a native sequence Fc region or amino acid sequence variant Fc region) of an antibody or antigen-binding fragment thereof. Examples of effector functions include Clq binding; complement dependent cytotoxicity; Fc receptor binding; antibody-dependent cell-mediated cytotoxicity (ADCC); phagocytosis; down regulation of cell surface receptors (e.g., B cell receptor; BCR), etc. An antibody or antigen-binding fragment thereof can also be “Fc silent,” which refers to antibodies or antigen-binding fragments that do not bind to Fc receptor or that do not have ADCC function.

Depending on the amino acid sequence of the constant domain of their heavy chains, antibodies and antigen-binding fragments thereof can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

As used herein, the terms “variable region” or “variable domain” are used interchangeably and are common in the art. The variable region typically refers to a portion of an antibody or antigen-binding fragments thereof, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and antigen-binding fragments thereof and are used in the binding and specificity of a particular antibody or antigen-binding fragment thereof for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody or antigen-binding fragment thereof with antigen. In some aspects, the variable region is a human variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

The terms “VL” and “VL domain” are used interchangeably to refer to the light chain variable region of an antibody or antigen-binding fragment thereof.

The terms “VH” and “VH domain” are used interchangeably to refer to the heavy chain variable region of an antibody or antigen-binding fragment thereof.

The term “Kabat numbering” and like terms are recognized in the art and refer to a system of numbering amino acid residues in the heavy and light chain variable regions of an antibody or an antigen-binding fragment thereof. In some aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

TABLE 1
CDR Numbering

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

As used herein, the term “constant region” or “constant domain” are interchangeable and have its meaning common in the art. The constant region is an antibody portion, e.g., a carboxyl terminal portion of a light and/or heavy chain which is not directly involved in binding of an antibody or antigen-binding fragment thereof to antigen but which can exhibit various effector functions, such as interaction with the Fc receptor. The constant region of an immunoglobulin molecule generally has a more conserved amino acid sequence relative to an immunoglobulin variable domain.

As used herein, the term “specifically binds” refers to the interaction between molecules wherein (i) the binding becomes saturated when the concentration of one of the molecules is increased with respect to the other molecule and (ii) the binding can be competed by the presence of an excess of one of the molecules of the interaction. A molecule (e.g., a binding domain such as an antibody or antigen-binding fragment thereof) that is capable of “specifically binding” to a target on the surface of an effector T cell may also bind to soluble forms of the target and/or the target in a different location (e.g., on the surface of another cell). A molecule (e.g., a binding domain such as an antibody or antigen-binding fragment thereof) that is capable of “specifically binding” to a target from one species (e.g., human) may also bind to that target from another species (e.g., cynomolgous monkey, mouse, and/or rat), but the extent of binding to an un-related target is less than about 10% of the binding to the target.

As used herein the term “immunospecifically binds” indicates that an antibody or antigen-binding fragment thereof binds to an epitope via its antigen-binding domain and that the binding entails some complementarity between the antigen binding domain and the epitope. Accordingly, an antibody that “immunospecifically binds” to a human protein, e.g., human PD1 may also bind to that protein from other species (e.g., cynomolgous monkey, mouse, and/or rat PD1) or to related proteins, but the extent of binding to an un-related protein is less than about 10% of the binding of the antibody to PD1 as measured. An antibody that “immunospecifically binds” an antigen of interest is one capable of binding that antigen with sufficient affinity such that the antibody is useful in targeting a cell expressing the antigen.

The terms “reduction” or “reduce” or “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some aspects, by “reduce” or “inhibit” is meant the ability to cause an overall decrease of 20% or greater. In some aspects, by “reduce” or “inhibit” is meant the ability to cause an overall decrease of 50% or greater. In some aspects, by “reduce” or “inhibit” is meant the ability to cause an overall decrease of 75%, 85%, 90%, 95%, or greater. In some aspects, the amount noted above is inhibited or decreased over a period of time, relative to a control over the same period of time.

A molecule that is an “inhibitor” of a target can decrease the activity, function, and/or amount of the target as compared to the activity, function, and/or amount of the target in the absence of the inhibitor. Thus a molecule, compound, or payload that is an “inhibitor of Cbl-b” can decrease the activity, function, and/or amount of Cbl-b as compared to the activity, function, and/or amount of Cbl-b in the absence of the molecule, compound, or payload.

A molecule that is an “agonist” of a target can increase the activity, function, and/or amount of the target as compared to the activity, function, and/or amount of the target in the absence of the agonist.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). It is understood that wherever aspects are described herein with the language “about” or “approximately,” a numeric value or range that is otherwise analogous but refers to the specific numeric value or range (without “about”) is also provided. The terms “administer,” “administering,” “administration,” and the like, as used herein, refer to methods that may be used to enable delivery of a drug, e.g., an activator of an effector T cell (e.g., a small molecule or an antibody drug conjugate) to a desired site of biological action. Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

The terms “antibody-drug conjugate” and “ADC” are used interchangeably and refer to an antibody or antigen-binding fragment thereof linked, e.g., covalently, to a therapeutic agent (sometimes referred to herein as agent, drug, or active pharmaceutical ingredient) or agents. In some aspects of the present disclosure, the biologically active molecule is an antibody-drug conjugate. In some aspects, an ADC comprises an antibody or antigen-binding fragment thereof covalently linked to a payload that is capable of activating an effector T cell. An ADC with a payload that is capable of activating an effector T cell can also be capable of activating one or more other cell types, e.g., one or more other T cell types, so long as it is capable of activating an effector T cell.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least about 70% identical, at least about 80% identical, at least about 90% identical, or at least about 95% identical to one another. In some aspects, two or more sequences are said to be “conserved” if they are at least about 30% identical, at least about 40% identical, at least about 50% identical, at least about 60% identical, at least about 70% identical, at least about 80% identical, at least about 90% identical, or at least about 95% identical to one another. Conservation of sequence can apply to the entire length of an polynucleotide or polypeptide or can apply to a portion, region or feature thereof.

As used herein, the terms “linking” and “conjugating” are used interchangeably and each refer to the covalent or non-covalent attachment of two or more moieties comprising one or more compounds capable of activating an effector T cell and a binding moiety. In some aspects the linking or conjugating can comprise a linker.

The term “amino acid sequence variant” refers to polypeptides having amino acid sequences that differ to some extent from a native sequence polypeptide. Ordinarily, amino acid sequence variants will possess at least about 70% sequence identity with at least one receptor binding domain of a native antibody or with at least one ligand binding domain of a native receptor, and typically, they will be at least about 80%, more typically, at least about 90% homologous by sequence with such receptor or ligand binding domains. The amino acid sequence variants possess substitutions, deletions, and/or insertions at certain positions within the amino acid sequence of the native amino acid sequence. Amino acids are designated by the conventional names, one-letter and three-letter codes.

“Sequence identity” is defined as the percentage of residues in the amino acid sequence variant that are identical after aligning the sequences and introducing gaps, if necessary, to achieve the maximum percent sequence identity. Methods and computer programs for the alignment are well known in the art. One such computer program is “Align 2,” authored by Genentech, Inc., which was filed with user documentation in the United States Copyright Office, Washington, D.C. 20559, on Dec. 10, 1991.

The terms “Fc receptor” or “FcR” are used to describe a receptor that binds to the Fc region of an antibody or antigen-binding fragment thereof. An exemplary FcR is a native sequence human FcR. Moreover, a FcR may be one which binds an IgG antibody or antigen-binding fragment thereof (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. Other FcRsare encompassed by the term “FcR” herein. The term also includes the neonatal receptor, FcRn, which is responsible for the transfer of maternal IgGs to the fetus.

“Complement dependent cytotoxicity” or “CDC” refers to the ability of a molecule to lyse a target in the presence of complement. The complement activation pathway is initiated by the binding of the first component of the complement system (Clq) to a molecule (e.g., an antibody or antigen-binding fragment thereof) complexed with a cognate antigen. To assess complement activation, a CDC assay can be performed.

The “light chains” of antibodies or antigen-binding fragments thereof from any vertebrate species can be assigned to one of two clearly distinct types, called kappa and lambda, based on the amino acid sequences of their constant domains.

An “isolated” antibody or antigen-binding fragment thereof is one which has been identified and separated and/or recovered from a component of its natural environment. Contaminant components of its natural environment are materials which would interfere with diagnostic or therapeutic uses for the antibody or antigen-binding fragment thereof, and may include enzymes, hormones, and other proteinaceous or nonproteinaceous solutes. In certain aspects, the antibody or antigen-binding fragment thereof will be purified (1) to greater than 95% by weight of antibody or antigen-binding fragment thereof as determined by the Lowry method, or more than 99% by weight, (2) to a degree sufficient to obtain at least 15 residues of N-terminal or internal amino acid sequence by use of a gas phase protein sequencer, or (3) to homogeneity by SDS-PAGE under reducing or nonreducing conditions using Coomassie blue or silver stain. Isolated antibody or antigen-binding fragment thereof includes the antibody or antigen-binding fragment thereof in situ within recombinant cells since at least one component of the antibody's or antigen-binding fragment thereof's natural environment will not be present. Ordinarily, however, an isolated antibody or antigen-binding fragment thereof will be prepared by at least one purification step.

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell. As used herein, the term “T cell” and “T lymphocytes” are interchangeable and refer to any lymphocytes produced or processed by the thymus gland. In some aspects, a T cell is a CD4+ T cell. In some aspects, a T cell is a CD8+ T cell. In some aspects, a T cell is a NKT cell.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein.

The term “therapeutically effective amount” or “therapeutically effective dosage” refers to an amount of an agent (e.g., a conjugate disclosed herein) that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the composition can, for example, (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer.

In some aspects, a “therapeutically effective amount” is the amount of the conjugate clinically proven to affect a significant decrease in cancer or slowing of progression (regression) of cancer, such as an advanced solid tumor. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the term “standard of care” refers to a treatment that is accepted by medical experts as a proper treatment for a certain type of disease and that is widely used by healthcare professionals. The term can be used interchangeably with any of the following terms: “best practice,” “standard medical care,” and “standard therapy.”

By way of example, an “anti-cancer agent” promotes cancer regression in a subject or prevents further tumor growth. In certain aspects, a therapeutically effective amount of the drug promotes cancer regression to the point of eliminating the cancer.

The terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

As used herein, the term “immune checkpoint inhibitor” refers to molecules that totally or partially reduce, inhibit, interfere with or modulate one or more checkpoint proteins. Checkpoint proteins regulate T cell activation or function. Numerous checkpoint proteins are known, such as CTLA-4 and its ligands CD80 and CD86; and PD1 with its ligands PDL1 and PDL2. Pardoll, D. M., Nat Rev Cancer 12 (4):252-64 (2012). These proteins are responsible for co-stimulatory or inhibitory interactions of T cell responses. Immune checkpoint proteins regulate and maintain self-tolerance and the duration and amplitude of physiological immune responses. Immune checkpoint inhibitors include antibodies and antigen-binding fragments thereof.

The terms “treat” or “treatment” refer to both therapeutic treatment and prophylactic or preventative measures, wherein the object is to prevent or slow down (lessen) an undesired physiological change or disorder, such as the development or spread of cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

Where not specifically designated, the stereocenters within the compounds described herein can be R- or S-stereochemistry and can be substituted as described elsewhere in the disclosure. It should be understood that when stereochemistry is not specified, the present disclosure encompasses all stereochemical isomeric forms, or mixtures thereof, which possess the ability to inhibit Cbl-b and/or activate T cells. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

### II. Cbl-b Inhibitors

In certain aspects, the present disclosure provides Cbl-b inhibitors of formula (I):

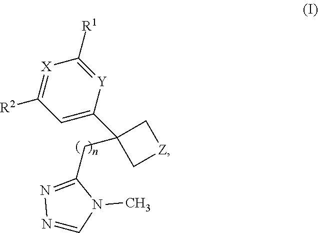

or a pharmaceutically acceptable salt thereof, wherein:
   n is 0, 1, or 2;
   X and Y are each independently CH or N;
   Z is selected from CH(CH₃), O, and SO₂; or
   Z is selected from CH(CH₃), NH, N(CH₃), O, and SO₂;
   R¹ is selected from hydrogen, —CN, —NHR^{z}, —R^{a}, —NR^{a}R^{b}, —OR^{a}, —NHC(O)R^{a}, —NHC(S)R^{a}, —NHC(O)NHR^{a}, —NHC(S)NHR^{a}, —SR^{a}, C₃-C₆cycloalkyl, and a 3- to 6-membered heterocyclyl ring; wherein:
      R^{z} is selected from

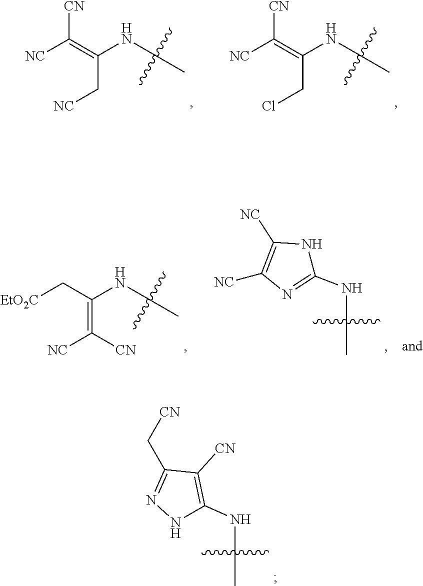 [SMILES: *NC(CC#N)=C(C#N)C#N ; *NC(CCl)=C(C#N)C#N ; *NC(CC(=O)OCC)=C(C#N)C#N ; *Nc1nc(C#N)c(C#N)[nH]1 ; *Nc1[nH]nc(CC#N)c1C#N]

R^{a} and R^{b} are independently selected from hydrogen, C₂-C₆alkenyl, C₁-C₆alkyl, amido(C₁-C₆alkyl), amino(C₁-C₆alkyl), azido(C₁-C₆alkyl), C₂-C₆alkynyl, carboxy(C₁-C₆alkyl), cyano(C₁-C₆alkyl), C₃-C₆cycloalkyl optionally substituted with a cyano group, dimethylamino(C₁-C₆alkyl), a 3- to 6-membered heterocyclyl ring, 3-6-membered heterocyclyl(C₁-C₃alkyl), hydroxy(C₁-C₆alkyl), methoxy(C₁-C₆alkyl), methylamino(C₁-C₆alkyl), NR^{c}R^{d}(C₁-C₆alkyl), HS(C₁-C₆alkyl), and CH₃S(C₁-C₆alkyl), wherein R^{c} and R^{d} are independently selected from hydrogen, C₂alkenylcarbonyl, and methyl; or,
R^{a} and R^{b}, together with the nitrogen atom to which they are attached, form a five- or six-membered ring optionally containing one additional nitrogen atom, wherein the ring is optionally substituted with one group selected from R^{a}, —C(O)R^{a}, —SO₂R^{a}, azido, and cyano;
wherein each C₃-C₆cycloalkyl, each 3- to 6-membered heterocyclyl ring, and the heterocyclyl part of the 3- to 6-membered heterocyclyl(C₁-C₃alkyl) ring are optionally substituted with one, two, or three groups independently selected from C₁-C₃alkyl, C₂alkynyl, amido, azido, carboxy, cyano, dimethylamino, hydroxy, methoxy, methylamino, HS—, and CH₃S—; and
R² is selected from

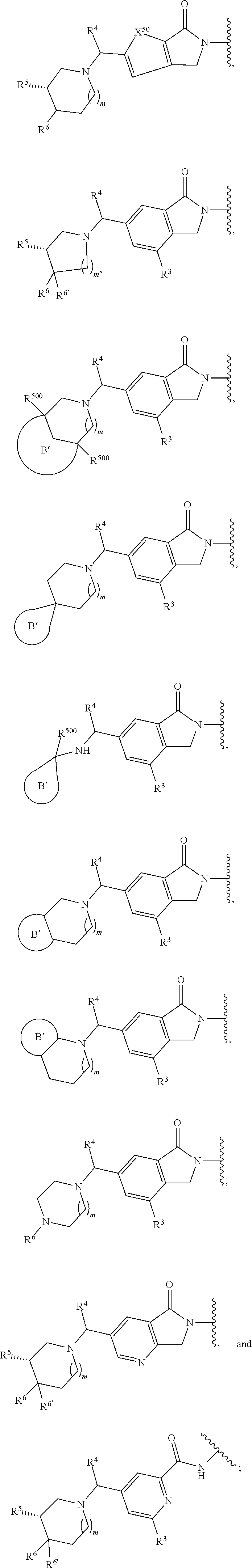

wherein
m is 0, 1, 2, or 3;
m″ is 0, 1, 2, 3, or 4;
B′ is a 3-7 membered saturated or unsaturated ring optionally containing one or two heteroatoms independently selected from nitrogen, oxygen, and sulfur; wherein the ring is optionally substituted with one or two substituents independently selected from —OH, —CH₂SH, CH₂SCH₃, —CH₂OH, —CH₂NH₂, and —CH₂NHCH₃;
each R⁵⁰⁰ is independently selected from hydrogen, C₁-C₆alkyl, halo, —OH, and —CH₂OH; and
X⁵⁰ is selected from O, NH, NCH₃, and S;
R³ is selected from hydrogen, acetyl, amino, C₁-C₆alkylamino, C₁-C₆alkylaminomethyl, C₁-C₆alkylcarbonyl, aminoC₁-C₆alkyl, aminocarbonyl, aminomethyl, carboxy, cyano, C₃cycloalkyl, formyl, hydroxy, hydroxyC₁-C₆alkyl, methoxy, oxazolyl, —SH, —SCH₃, —SOCH₃, —SO₂CH₃, —SO(═NH)CH₃, tetrazolyl, thiazolyl, and trifluoromethyl, wherein the C₃cycloalkyl is optionally substituted with a hydroxy group;
R⁴ is selected from hydrogen, methyl, —CH₂OH, —CH₂SH, and —CH₂SCH₃;
R⁵ is selected from hydrogen, hydroxy, —CH₂SH, —CH₂SCH₃, and methyl;
optionally provided that when R⁵ is hydroxy or methyl, and R⁴ is hydrogen, then R¹ is other than C₃-C₆cycloalkyl, a 3- to 6-membered heterocyclyl ring, hydroxy, hydroxy(C₁-C₆alkyl), —OR^{a}wherein R^{a} is C₁-C₆alkyl, a 3- to 6-membered heterocyclyl ring, or hydroxy(C₁-C₆alkyl); or —NR^{a}R^{b}, wherein R^{a} and R^{b} are independently selected from the group consisting of hydrogen, C₁-C₆alkyl, C₃-C₆cycloalkyl, hydroxy(C₁-C₆alkyl), a 3- to 6-membered heterocyclyl ring, or wherein R^{a} and R^{b}, together with the nitrogen atom to which they are attached, form a five- or six-membered ring optionally containing one additional nitrogen atom, wherein the ring is optionally substituted with one group selected from C₁-C₆alkyl or hydroxy(C₁-C₆alkyl); and
R⁶ and R^{6′} are independently selected from hydrogen, cyclopropyl, —CH₂OH, —CH₂SH, —CH₂SCH₃, and —CH₂R²⁰⁰, wherein R²⁰⁰ is a 3-7 membered saturated or unsaturated ring optionally containing one or two heteroatoms independently selected from nitrogen, oxygen, and sulfur.

In some aspects, R⁴ is in the “R” configuration. In some aspects, R⁴ is in the “S” configuration. In certain aspects, the present disclosure provides Cbl-b inhibitors of formula (IA-1):

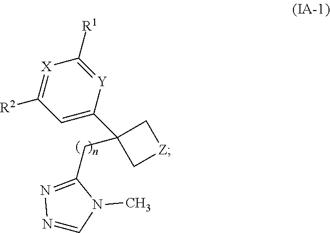

or a pharmaceutically acceptable salt thereof, wherein:
   n is 0, 1, or 2;
   X and Y are each independently CH or N;
   Z is selected from CH(CH₃), O, and SO₂;
   R¹ is selected from hydrogen, —CN, —NHR^{z}, —R^{a}, —NR^{a}R^{b}, —OR^{a}, —NHC(O)R^{a}, —NHC(S)R^{a}, —NHC(O)NHR^{a}, —NHC(S)NHR^{a}, —SR^{a}, C₃-C₆cycloalkyl, and a 3- to 6-membered heterocyclyl ring; wherein:
      R^{z} is selected from

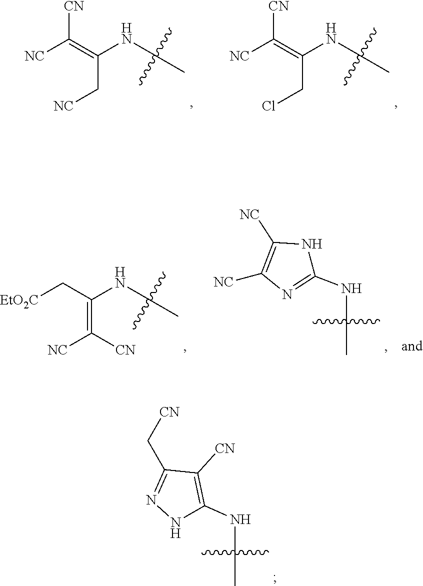 [SMILES: *NC(CC#N)=C(C#N)C#N ; *NC(CCl)=C(C#N)C#N ; *NC(CC(=O)OCC)=C(C#N)C#N ; *Nc1nc(C#N)c(C#N)[nH]1 ; *Nc1[nH]nc(CC#N)c1C#N]

R^{a} and R^{b} are independently selected from hydrogen, C₂-C₆alkenyl, C₁-C₆alkyl, amido(C₁-C₆alkyl), amino(C₁-C₆alkyl), azido(C₁-C₆alkyl), C₂-C₆alkynyl, carboxy(C₁-C₆alkyl), cyano(C₁-C₆alkyl), C₃-C₆cycloalkyl, dimethylamino(C₁-C₆alkyl), a 3-6-membered heterocyclyl ring, 3-6-membered heterocyclyl(C₁-C₃)alkyl, hydroxy(C₁-C₆alkyl), methoxy(C₁-C₆alkyl), methylamino(C₁-C₆alkyl), NR^{c}R^{d}(C₁-C₆alkyl), HS(C₁-C₆alkyl), and CH₃S(C₁-C₆alkyl), wherein R^{c} and R^{d} are independently selected from hydrogen, C₂alkenylcarbonyl, and methyl; or,
R^{a} and R^{b}, together with the nitrogen atom to which they are attached, form a five- or six-membered ring optionally containing one additional nitrogen atom, wherein the ring is optionally substituted with one group selected from R^{a}, —C(O)R^{a}, —SO₂R^{a}, azido, and cyano;
wherein each C₃-C₆cycloalkyl, each 3-6-membered heterocyclyl ring, and the heterocyclyl part of the 3-6 membered heterocyclyl(C₁-C₃alkyl) are optionally substituted with one, two, or three groups independently selected from C₁-C₃alkyl, C₂alkynyl, amido, azido, carboxy, cyano, dimethylamino, hydroxy, methoxy, methylamino, HS—, and CH₃S—; and
R² is

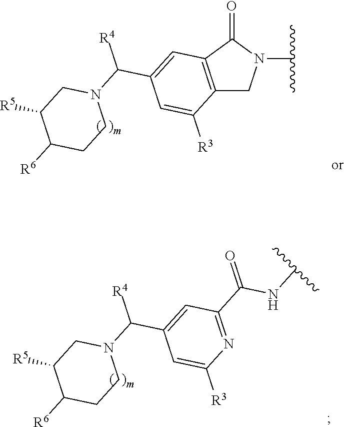

wherein
m is 0, 1, 2, or 3;
R³ is selected from hydrogen, acetyl, amino, C₁-C₆alkylamino, C₁-C₆alkylaminomethyl, aminoC₁-C₆alkyl, aminocarbonyl, aminomethyl, carboxy, cyano, C₃cycloalkyl, formyl, hydroxy, hydroxyC₁-C₆alkyl, methoxy, oxazolyl, —SH, —SCH₃, tetrazolyl, thiazolyl, and trifluoromethyl, wherein the C₃ cycloalkyl is optionally substituted with a hydroxy group;
R⁴ and R⁶ are independently selected from hydrogen, —CH₂SH, and —CH₂SCH₃; and
R⁵ is selected from hydroxy, —CH₂SH, —CH₂SCH₃, and methyl;
optionally provided that when R⁵ is hydroxy or methyl, and R⁴ is hydrogen, then R¹ is other than C₃-C₆cycloalkyl, a 3- to 6-membered heterocyclyl ring, hydroxy, hydroxy(C₁-C₆alkyl), —OR^{a} wherein R^{a} is C₁-C₆alkyl, a 3- to 6-membered heterocyclyl ring, or hydroxy(C₁-C₆alkyl); or —NR^{a}R^{b}, wherein R^{a} and R^{b} are independently selected from the group consisting of hydrogen, C₁-C₆alkyl, C₃-C₆cycloalkyl, hydroxy(C₁-C₆alkyl), a 3- to 6-membered heterocyclyl ring, or wherein R^{a} and R^{b}, together with the nitrogen atom to which they are attached, form a five- or six-membered ring optionally containing one additional nitrogen atom, wherein the ring is optionally substituted with one group selected from C₁-C₆alkyl or hydroxy(C₁-C₆alkyl).

In some aspects, R⁴ is in the “R” configuration. In some aspects, R⁴ is in the “S” configuration.

Excluded from compounds of formula (I) are those disclosed in WO2019/148005, WO2020/210508, and WO2021/021761.

In some aspects, the present disclosure provides a compound of formula (I), or a pharmaceutically acceptable salt thereof, wherein:
X is N;
Y is CH; and

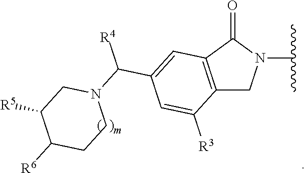

R² is

In some aspects, R⁴ is in the “R” configuration. In some aspects, R⁴ is in the “S” configuration.

In some aspects, the present disclosure provides a compound of formula (I), or a pharmaceutically acceptable salt thereof, wherein Z is O.

In some aspects, the present disclosure provides a compound of formula (I), or a pharmaceutically acceptable salt thereof, wherein Z is CH(CH₃).

In some aspects, the present disclosure provides a compound of formula (I), or a pharmaceutically acceptable salt thereof, wherein:
R³ is trifluoromethyl;
R⁴ and R⁶ are hydrogen; and
R⁵ is methyl.

In some aspects, the present disclosure provides a compound of formula (I), or a pharmaceutically acceptable salt thereof, wherein R¹ is selected from —NR^{a}R^{b}, —NHC(O)R^{a}, —NHC(S)NHR^{a}, and —SR^{a}

In some aspects, the present disclosure provides a compound of formula (I), or a pharmaceutically acceptable salt thereof, wherein R¹ is —SCH₂CH₃.

In some aspects, the present disclosure provides a compound of formula (I), or a pharmaceutically acceptable salt thereof, wherein R¹ is —NH(CH₂)₂CN.

In some aspects, the present disclosure provides a compound of formula (I), or a pharmaceutically acceptable salt thereof, wherein R¹ is —NH(CH₂)₂N₃.

As used herein, the term “acetyl” refers to —C(O)CH₃.

As used herein, the term “C₂alkenyl” refers to —CH═CH₂.

As used herein, the term “C₂-C₆alkenyl” refers to a group derived from a straight or branched chain hydrocarbon containing from two to six carbon atoms and containing at least one double bond.

As used herein, the term “C₂alkenylcarbonyl” refers to a C₂alkenyl group attached to the parent molecular moiety through a carbonyl group.

As used herein, the term “C₁-C₆alkoxy,” as used herein, refers to a C₁-C₆alkyl group attached to the parent molecular moiety through an oxygen atom.

As used herein, the term “C₁-C₆alkoxyC₁-C₆alkyl” refers to a C₁-C₆alkoxy group attached to the parent molecular moiety through a C₁-C₆alkyl group.

As used herein, the term “C₁-C₆alkyl” refers to a group derived from a straight or branched chain saturated hydrocarbon containing from one to six carbon atoms.

As used herein, the term “C₁-C₆alkylamino” refers to —NHR, wherein R is a C₁-C₆alkyl group.

As used herein, the term “C₁-C₆alkylaminomethyl” refers to a C₁-C₆alkylamino group attached to the parent molecular moiety through a methylene group.

As used herein, the term “C₂alkynyl” refers to —C≡C—H.

As used herein, the term “C₂-C₆alkynyl” refers to a group derived from a straight or branched chain hydrocarbon containing from two to six carbon atoms and containing at least one double bond.

As used herein, the term “amido” refers to —C(O)NH₂.

As used herein, the term “amido(C₁-C₆alkyl)” refers to an amido group attached to the parent molecular moiety through a C₁-C₆alkyl group.

As used herein, the term “amino” refers to —NH₂.

As used herein, the term “amino(C₁-C₆alkyl)” refers to an amino group attached to the parent molecular moiety through a C₁-C₆alkyl group.

As used herein, the term “aminocarbonyl” refers to an amino group attached to the parent molecular moiety through a carbonyl group.

As used herein, the term “aminomethyl” refers to an amino group attached to the parent molecular moiety through a methylene group.

As used herein, the term “azido” refers to —N₃.

As used herein, the term “azido(C₁-C₆alkyl)” refers to an azido group attached to the parent molecular moiety through a C₁-C₆alkyl group.

As used herein, the term “carbonyl” refers to —C(O)—.

As used herein, the term “carboxy” refers to —CO₂H.

As used herein, the term “carboxy(C₁-C₆alkyl)” refers to a carboxy group attached to the parent molecular moiety through a C₁-C₆alkyl.

As used herein, the term “cyano” refers to —CN.

As used herein, the term “cyano(C₁-C₆alkyl)” refers to a cyano group attached to the parent molecular moiety through a C₁-C₆alkyl group.

As used herein, the term “C₃cycloalkyl” refers to a cyclopropyl group.

As used herein, the term “C₃-C₆cycloalkyl” refers to a saturated monocyclic, hydrocarbon ring system having three to six carbon atoms and zero heteroatoms. Representative examples of cycloalkyl groups include, but are not limited to, cyclobutyl, cyclopentyl, and cyclohexyl.

As used herein, the term “dimethylamino(C₁-C₆alkyl)” refers to a dimethylamino group attached to the parent molecular moiety through a C₁-C₆alkyl group.

As used herein, the term “formyl” refers to —CHO.

As used herein, the term “halo” refers to F, Cl, Br, or I.

As used herein, the term “3- to 6-membered heterocyclyl” refers to a three-, four-, five-, or six-membered saturated or unsaturated, containing one, two, three, or four heteroatoms independently selected from nitrogen, oxygen, and sulfur. Representative examples of 3- to 6-membered heterocyclyl groups include, but are not limited to, oxetane, pyrrolidine, thiazole, pyridine, and piperidine.

As used herein, the term “3- to 6-membered heterocyclyl(C₁-C₃alkyl)” refers to a 3- to 6-membered heterocyclyl ring attached to the parent molecular moiety through a C₁-C₃alkyl.

As used herein, the term “hydroxy” refers to —OH.

As used herein, the term “hydroxyC₁-C₆alkyl” refers to a C₁-C₆alkyl group substituted with one, two, or three hydroxy groups.

As used herein, the term “methoxy(C₁-C₆alkyl)” refers to a methoxy group attached to the parent molecular moiety through a C₁-C₆alkyl group.

As used herein, the term “methylamino(C₁-C₆alkyl)” refers to a methylamino group attached to the parent molecular moiety through a C₁-C₆alkyl group.

As used herein, the term “NR^{c}Rd(C₁-C₆alkyl)” refers to an NR^{c}R^{d} group attached to the parent molecular moiety through a C₁-C₆alkyl group. R^{c} and R^{d} are as defined herein.

As used herein, the term “HS(C₁-C₆alkyl)” refers to an —SH group attached to the parent molecular moiety through a C₁-C₆alkyl group.

In some aspects, the compound of formula (I) is a compound selected from the group consisting of

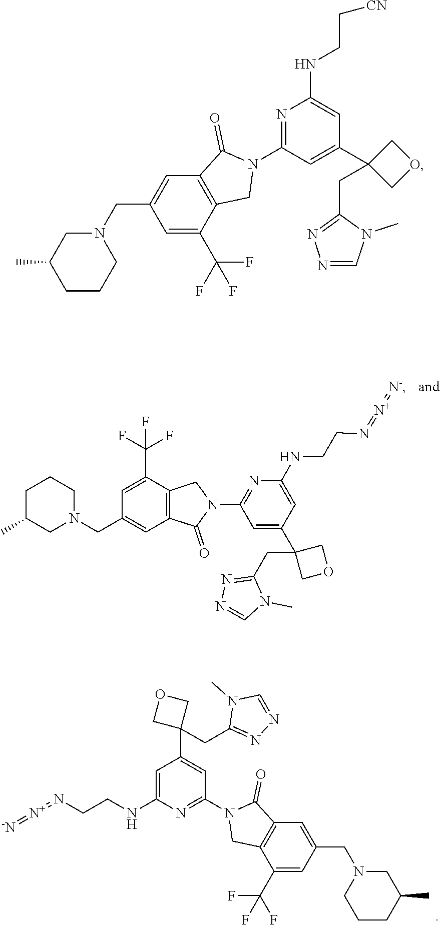

In some aspects, the compound of formula (I) is capable of treating cancer in a subject. In some aspects, the compound of formula (I) is capable of increasing effector T cell activity and/or effector T cell proliferation. In some aspects, the compound of formula (I) is capable of increasing migration of effector T cells to a tumor cells. In some aspects, the compound of formula (I) is capable of reducing effector T cell exhaustion.

In some aspects, the compound of formula (I) competitively inhibits binding of a probe comprised of BODIPY-FL fluorophore conjugated to a Cbl-b inhibitor (see Example 54 in WO20200264398) to Cbl-b, e.g., as assessed using the assay as described herein in Example 2. In some aspects, the compound of formula (I) competitively inhibits with an IC50 of about 1 nM to about 5 nM. In some aspects, the compound of formula (I) competitively inhibits with an IC50 of about 5.01 nM to about 20 nM. In some aspects, the compound of formula (I) competitively inhibits with an IC50 of about 20.01 nM to about 100 nM. In some aspects, the compound of formula (I) competitively inhibits with an IC50 of about 100.01 nM to about 1 mM.

In some aspects, the compound of formula (I) is capable of increasing IL-2 secretion from T cells, e.g., as assessed using the assay as described herein in Example 3. In some aspects, the compound of formula (I) increases IL-2 secretion over background by about 0.8 to about 1.4 fold. In some aspects, the compound of formula (I) increases IL-2 secretion over background by about 0.31 to about 0.79 fold. In some aspects, the compound of formula (I) increases IL-2 secretion over background by about 0.1 to about 0.3 fold.

In some aspects, the compound of formula (I) is capable of increasing IFN-y secretion from T cells, e.g., as assessed using the assay as described herein in Example 3. In some aspects, the compound of formula (I) increases IFN-y secretion over background by about 1.1 to about 2 fold. In some aspects, the compound of formula (I) increases IFN-y secretion over background by about 0.31 to about 1 fold. In some aspects, the compound of formula (I) increases IFN-y secretion over background by about 0.1 to about 0.3 fold.

In some aspects, the compound of formula (I) is capable of increasing CD69 levels, e.g., as assessed using the assay as described herein in Example 3. In some aspects, the compound of formula (I) increases CD69 levels over background by about 1.11 to about 1.5 fold. In some aspects, the compound of formula (I) increases CD69 levels over background by about 0.81 to about 1.10 fold In some aspects, the compound of formula (I) increases CD69 levels over background by about 0.81 to about 1.1 fold. In some aspects, the compound of formula (I) increases CD69 levels over background by about 0.61 to about 0.8 fold. In some aspects, the compound of formula (I) increases CD69 levels over background by about 0.3 to about 0.6 fold.

In some aspects, the compound of formula (I) interacts with His152 of human Cbl-b. In some aspects, the compound of formula (I) comprises a group at R¹ that comprises a terminal nitrogen atom. In some aspects, the distance between the terminal nitrogen atom of group R¹ and His152 of Cbl-b is about 1 to about 5 angstroms in a crystal structure of the compound and human Cbl-b. In some aspects, the distance between the terminal nitrogen atom of group R¹ and His152 of Cbl-b is about 1 to about 4 angstroms in co-crystal structure of the compound and human Cbl-b. In some aspects, the distance between the terminal nitrogen atom of group R¹ and His152 of Cbl-b is about 2 to about 5 angstroms in a co-crystal structure of the compound and human Cbl-b. In some aspects, the distance between the terminal nitrogen atom of group R¹ and His152 of Cbl-b is about 2 to about 4 angstroms in a co-crystal structure of the compound and human Cbl-b. In some aspects, the distance between the terminal nitrogen atom of group R¹ and His152 of Cbl-b is about 2.5 to about 5 angstroms in a co-crystal structure of the compound and human Cbl-b. In some aspects, the distance between the terminal nitrogen atom of group R¹ and His152 of Cbl-b is about 2.5 to about 4 angstroms in a co-crystal structure of the compound and human Cbl-b.

### III. Conjugates

The present disclosure provides conjugates comprising a binding moiety (e.g., antibody or antigen-binding fragment thereof) that is capable of specifically binding to a target on the surface of an effector T cell and a payload that is capable of activating an effector T cell. The binding moiety can be directly attached to the payload or can be attached to the payload through a linker. In certain aspects, the payload can be attached to the linker or binding moiety through a covalent bond.

In certain aspects, where the payload is a compound of formula (I):

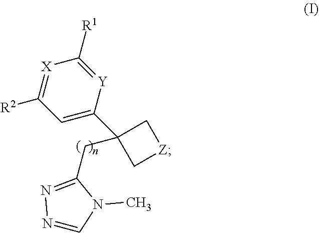

the compound of formula (I) can be attached to the linker through a covalent bond to a heteratom in group R¹.

In some aspects, where the payload is a compound of formula (Ia):

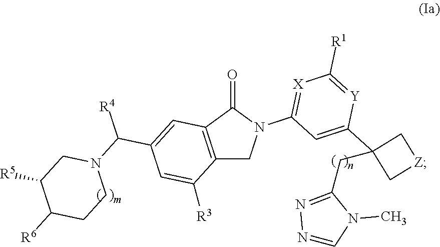

the compound of formula (Ia) can be attached to the linker through a covalent bond to R¹, R³, R⁴, R⁵, R⁶, or the nitrogen atom of the piperidine ring.

In some aspects, R⁴ is in the “R” configuration. In some aspects, R⁴ is in the “S” configuration.

In some aspects, where the payload is a compound of formula (Ib):

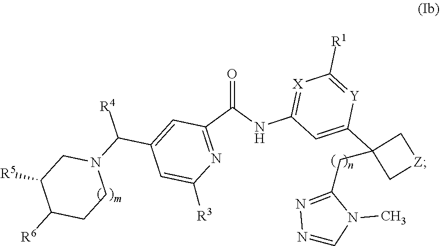

the compound of formula (Ib) can be attached to the linker through a covalent bond to R¹, R³, R⁴, R⁵, R⁶, or the nitrogen atom of the piperidine ring.

In some aspects, R⁴ is in the “R” configuration. In some aspects, R⁴ is in the “S” configuration.

In some aspects, where the payload is a compound of formula (Ic):

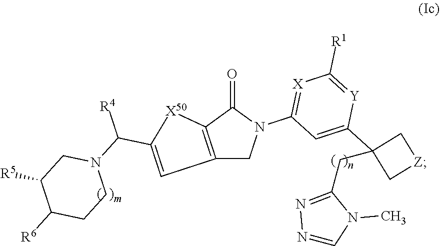

the compound of formula (Ic) can be attached to the linker through a covalent bond to R¹, R³, R⁴, R⁵, R⁶, or the nitrogen atom of the piperidine ring.

In some aspects, R⁴ is in the “R” configuration. In some aspects, R⁴ is in the “S” configuration.

In some aspects, where the payload is a compound of formula (Id):

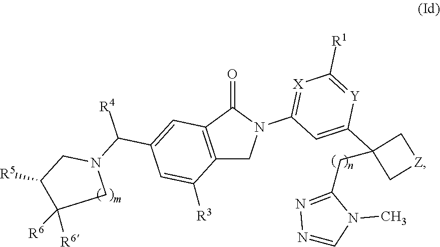

the compound of formula (Id) can be attached to the linker through a covalent bond to R¹, R³, R⁴, R⁵, R⁶, R^{6′}, or the nitrogen atom of the piperidine ring.

In some aspects, R⁴ is in the “R” configuration. In some aspects, R⁴ is in the “S” configuration.

In some aspects, where the payload is a compound of formula (Ie):

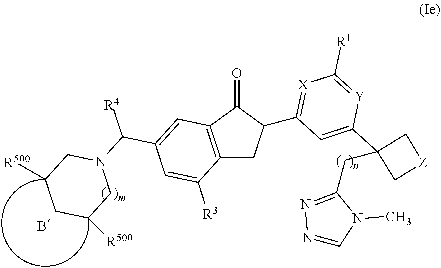

the compound of formula (Ie) can be attached to the linker through a covalent bond to R¹, R³, R⁴, R⁵⁰⁰, B′, or the nitrogen atom of the piperidine ring.

In some aspects, R⁴ is in the “R” configuration. In some aspects, R⁴ is in the “S” configuration.

In some aspects, where the payload is a compound of formula (If):

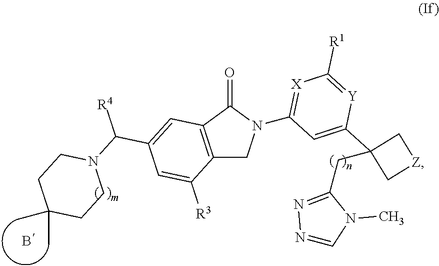

the compound of formula (If) can be attached to the linker through a covalent bond to R¹, R³, R⁴, B′, or the nitrogen atom of the piperidine ring.

In some aspects, R⁴ is in the “R” configuration. In some aspects, R⁴ is in the “S” configuration.

In some aspects, where the payload is a compound of formula (Ig):

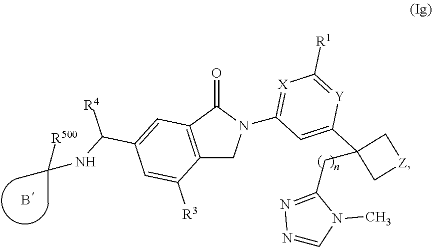

the compound of formula (Ig) can be attached to the linker through a covalent bond to R¹, R³, R⁴, R⁵⁰⁰, B′, or the nitrogen atom attached to B′.

In some aspects, R⁴ is in the “R” configuration. In some aspects, R⁴ is in the “S” configuration.

In some aspects, where the payload is a compound of formula (Ih):

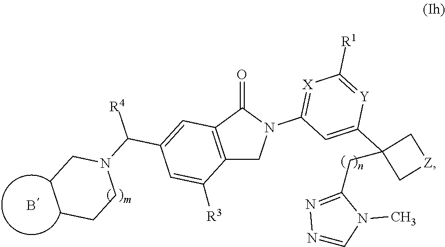

the compound of formula (Ig) can be attached to the linker through a covalent bond to R¹, R³, R⁴, B′, or the nitrogen atom of the piperidine ring.

In some aspects, R⁴ is in the “R” configuration. In some aspects, R⁴ is in the “S” configuration.

In some aspects, where the payload is a compound of formula (Ii):

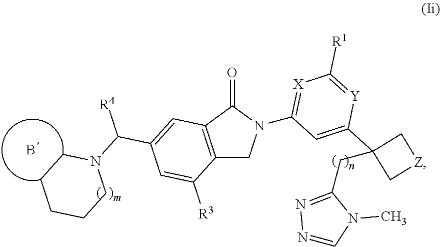

the compound of formula (Ii) can be attached to the linker through a covalent bond to R¹, R³, R⁴, B′, or the nitrogen atom of the piperidine ring.

In some aspects, R⁴ is in the “R” configuration. In some aspects, R⁴ is in the “S” configuration.

In some aspects, where the payload is a compound of formula (Ij):

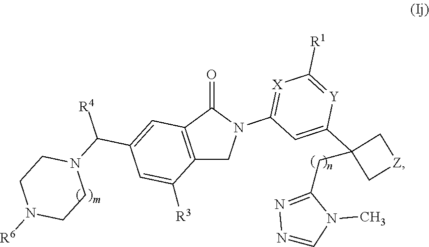

the compound of formula (Ii) can be attached to the linker through a covalent bond to R¹, R³, R⁴, B′, or either nitrogen atom of the piperazine ring.

In some aspects, R⁴ is in the “R” configuration. In some aspects, R⁴ is in the “S” configuration.

In some aspects, where the payload is a compound of formula (Ik):

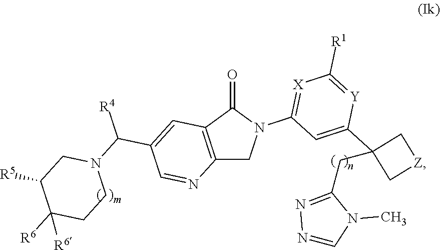

the compound of formula (Ik) can be attached to the linker through a covalent bond to R¹, R³, R⁴, R⁶, R^{6′}, or the nitrogen atom of the piperidine ring.

In some aspects, R⁴ is in the “R” configuration. In some aspects, R⁴ is in the “S” configuration.

In some aspects, where the payload is a compound of formula (Il):

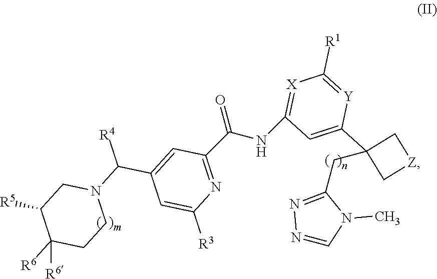

the compound of formula (Ik) can be attached to the linker through a covalent bond to R¹, R³, R⁴, R⁶, R^{6′}, or the nitrogen atom of the piperidine ring.

In some aspects, R⁴ is in the “R” configuration. In some aspects, R⁴ is in the “S” configuration.

In some aspects, a conjugate provided herein comprises a binding moiety (e.g., an antibody or antigen-binding fragment thereof) that is capable of specifically binding to PD1 and a payload that is capable of activating an effector T cell. In some aspects, a conjugate provided herein comprises a binding moiety (e.g., an antibody or antigen-binding fragment thereof) that is capable of specifically binding to PD1 and a payload that is an inhibitor of Casitas B-lineage lymphoma proto-oncogene b (Cbl-b).

In some aspects, a conjugate provided herein comprises a binding moiety (e.g., an antibody or antigen-binding fragment thereof) that is capable of specifically binding to CD25 (IL2RA) and a payload that is capable of activating an effector T cell. In some aspects, a conjugate provided herein comprises a binding moiety (e.g., an antibody or antigen-binding fragment thereof) that is capable of specifically binding to PD1 and a payload that is an inhibitor of Casitas B-lineage lymphoma proto-oncogene b (Cbl-b).

In some aspects, a conjugate provided herein comprises a binding moiety (e.g., an antibody or antigen-binding fragment thereof) that is capable of specifically binding to PD1 and a payload that is an inhibitor of Casitas B-lineage lymphoma proto-oncogene b (Cbl-b).

In some aspects, a conjugate provided herein has formula (II): Bm-[L-P]ₐ (II), wherein a is an integer from 1 to 50; P is a payload; L is a linker; and Bm is a binding moiety (e.g., antibody or antigen-binding fragment thereof) In some aspects, a is about 1 to about 40. In some aspects, a is about 1 to about 10. In some aspects, a is about 2 to about 8.

In some aspects, provided herein is a composition (e.g., a pharmaceutical composition) comprising a conjugate (e.g., at least 2 conjugates) provided herein. In some aspects, the composition comprises an average of about 1 to about 50 payloads per binding moiety. In some aspects, the composition comprises an average of about 1 to about 10 payloads per binding moiety. In some aspects, the composition comprises an average of about 2 to about 8 payloads per binding moieties.

In some aspects, a conjugate or composition provided herein is capable of treating cancer in a subject. In some aspects, a conjugate or composition provided herein is capable of increasing effector T cell activity and/or effector T cell proliferation. In some aspects, a conjugate or composition provided herein is capable of increasing migration of effector T cells to a tumor cells. In some aspects, a conjugate or composition provided herein is capable of reducing effector T cell exhaustion. In some aspects, a conjugate or composition provided herein is capable of delivering a payload that is capable of activating an effector T cell to an effector T cell.

In some aspects, a conjugate or composition provided herein is capable of blocking PD1/PDL1, e.g. as measured using the assay as described herein in Example 7. In some aspects, the conjugate or composition results in about a 3.51 to about a 5.49 fold blockade of PD1/PDL1 over background. In some aspects, the conjugate or composition results in about a 2.5 to about a 3.5 fold blockade of PD1/PDL1 over background. In some aspects, the conjugate or composition results in about a 2 to about a 2.49 fold blockade of PD1/PDL1 over background. In some aspects, the conjugate or composition results in about a 1.5 to about a 1.99 fold blockade of PD1/PDL1 over background. In some aspects, the conjugate or composition results in about a 1.1 to about a 1.49 fold blockade of PD1/PDL1 over background.

In some aspects, a conjugate or composition provided herein is capable of increasing IFN-y secretion from T cells, e.g., as assessed using the assay as described herein in Example 7. In some aspects, the conjugate or composition increases IFN-y secretion over background by about 1 to about 1.4 fold. In some aspects, the conjugate or composition increases IFN-y secretion over background by about 1 to about 1.49 fold. In some aspects, the conjugate or composition increases IFN-y secretion over background by about 1.5 to about 1.99 fold. In some aspects, the conjugate or composition increases IFN-y secretion over background by about 2 to about 2.49 fold. In some aspects, the conjugate or composition increases IFN-y secretion over background by about 2.5 to about 3 fold.

In some aspects, a conjugate or composition provided herein is capable of increasing IL-2 secretion from T cells, e.g., as assessed using the assay as described herein in Example 7. In some aspects, the conjugate or composition increases IL-2 secretion over background by about 2 to about 4 fold. In some aspects, the conjugate or composition increases IL-2 secretion over background by about 4 to about 4.99 fold. In some aspects, the conjugate or composition increases IL-2 secretion over background by about 5 to about 5.99 fold. In some aspects, the conjugate or composition increases IL-2 secretion over background by about 6 to about 8 fold.

### III.A. Payloads

The conjugates of the present disclosure can comprise a payload linked to a binding moiety (i.e., “Bm”). The payload can be a molecule (e.g., small molecule, peptide, or nucleic acid) that is capable of activating an effector T cell. In some aspects, a payload is capable of promoting proliferation, survival, and/or migration of an effector T cell, promoting production of effector cytokines and/or cytotoxic mediators from an effector T cell, and/or reducing exhaustion of an effector T cell.

In some aspects, a payload is an inhibitor of Casitas B-lineage lymphoma proto-oncogene b (Cbl-b), hematopoietic progenitor kinase 1 (HPK-1), phosphoinositide-3-kinase gamma (PI3K7), a mitogen-activated protein kinase (MAPK) pathway protein (e.g., MEK and/or B-raf), CXCR4, or CCR5, or an agonist of toll-like receptor 7 (TLR-7), toll-like receptor 8 (TLR-8), or stimulator of interferon genes (STING).

In some aspects, a payload is an inhibitor of Cbl-b. In some aspects, the payload in a conjugate provided herein is a compound of formula (I) as above, which is attached to the binding moiety or the linker through a covalent bond. In some aspects, the payload is selected from the group consisting of Compound 146, Compound 147, and Compound 148. In some aspects, the payload is NX-1607.

In some aspects, a payload is an agonist of toll-like receptor 7 (TLR-7) and/or toll-like receptor 8 (TLR-8). Agonists of TLR-7 and/or TLR-8 are known in the art and include, for example T785 (as discussed e.g., in Ackerman S., et al., Nature Cancer 2: 18-33 (2021), which is herein incorporated by reference in its entirety) and MEDI9197 (as discussed e.g., in Huck B., et al., Angew. Chem. Int. Ed. 57: 4412-4428 (2018), which is herein incorporated by reference in its entirety.)

In some aspects, a payload is an inhibitor of hematopoietic progenitor kinase 1 (HPK-1). Inhibitors of HPK-1 are known in the art and reviewed, for example, in Linney, I. D. and Kaila N., Expert Opin. Ther. Pat. 31 (10): 893-910 (2021), which is herein incorporated by reference in its entirety.

In some aspects, a payload is an agonist of STING. Agonists of STING, such as (R,R)-52-CDA, ADU-S100, MIW815 (Aduro BioTech/Novartis) and the cyclic dinucleotide MK-1454, are known in the art and discussed, for example in Huck B., et al., Angew. Chem. Int. Ed 57: 4412-4428 (2018), which is herein incorporated by reference in its entirety.)

In some aspects, a payload is an inhibitor of PI3K7. Inhibitors of PI3K7, such as IPI-549, are known in the art and discussed, for example in Huck B., et al., Angew. Chem. Int. Ed 57: 4412-4428 (2018), which is herein incorporated by reference in its entirety.)

In some aspects, a payload is an inhibitor of a mitogen-activated protein kinase (MAPK) pathway protein (e.g, MEK and/or B-raf). Inhibitors of MAPK pathway proteins, such as trametinib, cobimetinib, binimetinib, dabrafenib, vemurafenib, and encorafenib, are known in the art and discussed, for example in Huck B., et al., Angew. Chem. Int. Ed 57: 4412-4428 (2018), which is herein incorporated by reference in its entirety.)

In some aspects, a payload is an inhibitor of CCR4 and/or CCR5. Inhibitors of CCR4 and/or CCR5, such as BL-8040, X4P-001, LY2510924, and BMS-813160, are known in the art and discussed, for example in Huck B., et al., Angew. Chem. Int. Ed 57: 4412-4428 (2018), which is herein incorporated by reference in its entirety.)

In some aspects, a payload in a conjugate provided herein is a small molecule, a peptide, or a nucleotide (e.g., an siRNA). In some aspects, a payload in a conjugate provided herein is a small molecule. In some aspects, a payload in a conjugate provided herein is a peptide. Exemplary peptide payloads are disclosed, for example in EP Publication No. EP3254701, which is herein incorporated by reference in its entirety. In some aspects, a payload in a conjugate provided herein is a nucleotide (e.g., an siRNA). Exemplary nucleotide payloads are disclosed, for example in U.S. Publication No. US20150313931, which is herein incorporated by reference in its entirety.

### III.B. Linkers

The conjugates of the present disclosure can comprise a linker. As used herein, the term “linker” refers to any chemical moiety capable of connecting the binding moiety (i.e., “Bm”) to the payload (“P”).

In certain aspects, the linker can contain a heterobifunctional group. In the present disclosure, the term “heterobifunctional group” refers to a chemical moiety that connects the linker of which it is a part to the binding moiety. Heterobifunctional groups are characterized as having different reactive groups at either end of the chemical moiety. Attachment to the binding moiety can be accomplished through chemical or enzymatic conjugation, or a combination of both. Chemical conjugation involves the controlled reaction of accessible amino acid residues on the surface of the binding moiety with a reaction handle on the heterobifunctional group. Examples of chemical conjugation include, but are not limited to, lysine amide coupling, cysteine coupling, and coupling via a non-natural amino acid incorporated by genetic engineering, wherein non-natural amino acid residues with a desired reaction handle are installed onto the binding moiety. In enzymatic conjugation, an enzyme mediates the coupling of the linker with an accessible amino residue on the binding moiety. Examples of enzymatic conjugation include, but are not limited to, transpeptidation using sortase, transpeptidation using microbial transglutaminase, and N-glycan engineering. Chemical conjugation and enzymatic conjugation may also be used sequentially. For example, enzymatic conjugation can also be used for installing unique reaction handles on the binding moiety to be utilized in subsequent chemical conjugation.

In some aspects, the heterobifunctional group is selected from:

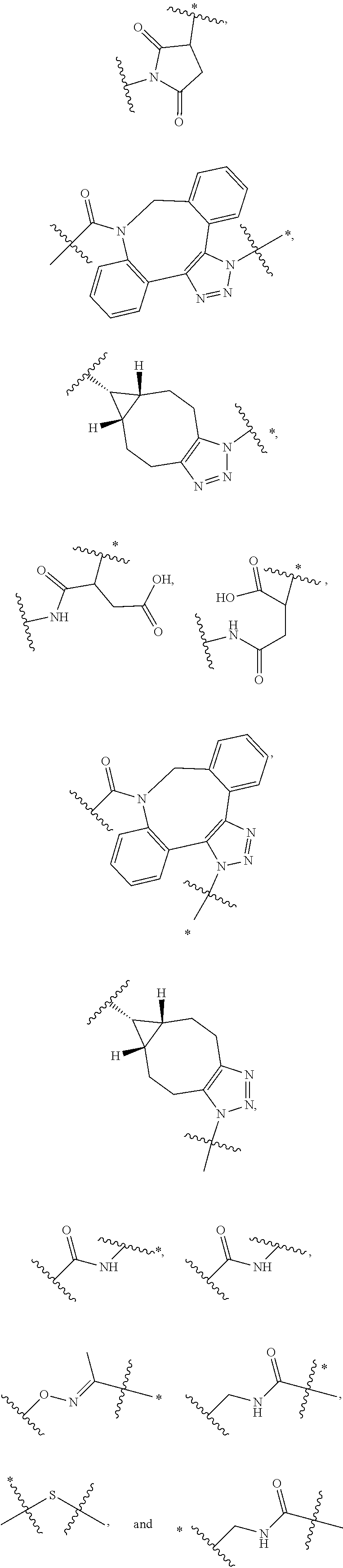

wherein
[symbol] is the point of attachment to the remaining portion of the linker; and
[symbol] the point of attachment to Bm.

In certain aspects, linker “L” is non-cleavable. As used here, the term “non-cleavable linker” is any chemical moiety that is capable of linking the binding to the payload in a stable, covalent manner and does not fall under the categories defined herein as “cleavable linkers”. Thus, non-cleavable linkers are substantially resistant to acid-induced cleavage, light-induced cleavage, bioreductive cleavage, peptidase-induced cleavage, esterase-induced cleavage, and disulfide bond cleavage. “Substantially resistant to cleavage” means that the chemical bond in the linker or adjoining the linker in at least 80%, preferably at least 85%, more preferably at least 90%, even more preferably at least 95%, and most preferably at least 99% of the conjugate population remains non-cleavable by an acid, a photolabile-cleaving agent, a bioreductive agent, a peptidase, an esterase, or a chemical or a physiological compound that cleaves the chemical bond (for example, a disulfide bond) in a cleavable linker, for within a few hours to several days of treatment with any of the agents described above. In certain aspects the linker is not susceptible to acid-induced cleavage, photo-induced cleavage, bioreductive cleavage, enzymatic cleavage, or the like, at conditions under which the payload and/or binding moiety can remain active. ADC catabolites generated from non-cleavable linkers contain a residual amino acid from the antibody. These catabolites can exert unique and unexpected properties in the target cells to which they are delivered.

A person of ordinary skill in the art would readily distinguish non-cleavable from cleavable linkers.

Examples of non-cleavable linkers include, but are not limited to, SMCC (succinimidyl 4-(N-maleimidomethyl)cyclohexane-1-carboxylate) linkers, succinimide thioether linkers, and linkers such as:

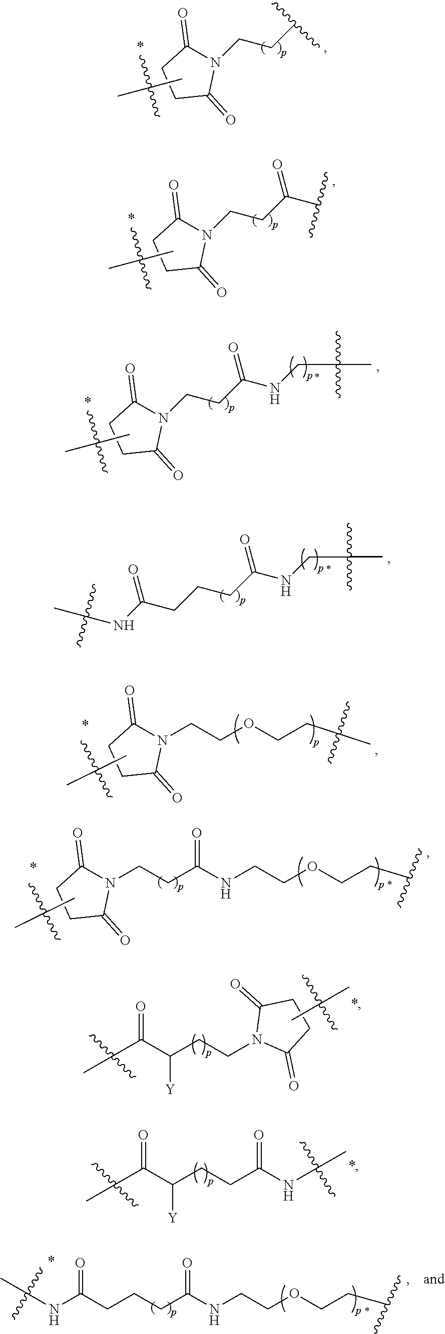

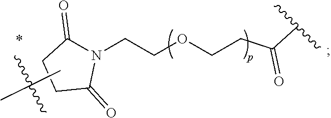

wherein:
   p is an integer from 1 to 10;
   p* is an integer from 1 to 10;
   Y is selected from hydrogen, and C₁-C₆alkyl;
      [symbol] is the point of attachment to the payload; and
      [symbol] is the point of attachment to the binding moiety.

In some aspects, the linker is:

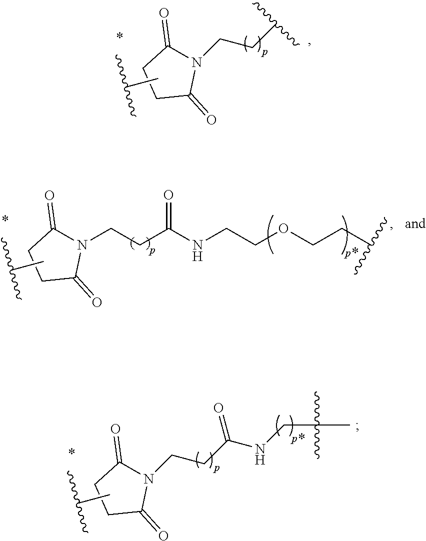

wherein:
   p is an integer from 1 to 10;
   p* is an integer from 1 to 10;
   [symbol] is the point of attachment to the payload; and
   [symbol] is the point of attachment to the binding moiety.

In certain aspects the linker can be cleavable. In some aspects, the linker can be susceptible to acid-induced cleavage, photo-induced cleavage, bioreductive cleavage, enzymatic cleavage, or the like, at conditions under which the payload and/or binding moiety can remain active.

In some aspects, the cleavable linker can be cleaved enzymatically. In some aspects, the cleavable linker can be cleaved by a protease, peptidase, esterase, beta-gluroronidase, glycosidase, phosphodiesterase, phosphatase, pyrophosphatase, or lipase.

In some aspects, the cleavable linker can be cleaved by a protease. Examples of proteases include, but are not limited to, cathepsin B, VAGP tetrapeptide, and the like.

In certain aspects, the cleavable linker contains a peptide. In some aspects, the peptide is the site of cleavage of the linker, thereby facilitating release of the drug upon exposure to intracellular proteases, such as lysosomal enzymes. Peptides can be designed and optimized for enzymatic cleavage by a particular enzyme, for example, a tumor-associated protease, cathepsin B, C and D, or a plasmin protease. Examples of peptides having two amino acids include, but are not limited to, alanine-alanine (ala-ala), valine-alanine (val-ala), valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Examples of peptides having three amino acids include, but are not limited to, glycine-valine-citrulline (gly-val-cit), aspartic acid-valine-citrulline (asp-val-cit), alanine-alanine-asparagine (ala-ala-asn), alanine-phenylalanine-lysine (ala-phe-lys), glycine-glycine-phenylalanine (gly-gly-phe), and glycine-glycine-glycine (gly-gly-gly). Examples of peptides having four amino acids include, but are not limited to, glycine-glycine-valine-citrulline (gly-gly-val-cit) and glycine-glycine-phenylalanine-glycine (gly-gly-phe-gly). The amino acid combinations above can also be present in the reverse order (i.e., cit-val).

The peptides of the present disclosure can comprise L- or D-isomers of amino acid residues. The term “naturally-occurring amino acid” refers to Ala, Asp, Asx, Cit, Cys, Glu, Phe, Glx, Gly, His, Ile, Lys, Leu, Met, Asn, Pro, Gln, Arg, Ser, Thr, Val, Trp, and Tyr. “D-” designates an amino acid having the “D” (dextrorotary) configuration, as opposed to the configuration in the naturally occurring (“L-”) amino acids. The amino acids described herein can be purchased commercially (Sigma Chemical Co., Advanced Chemtech) or synthesized using methods known in the art.

In certain aspects, the linker (“L”) is a protease cleavable linker selected from

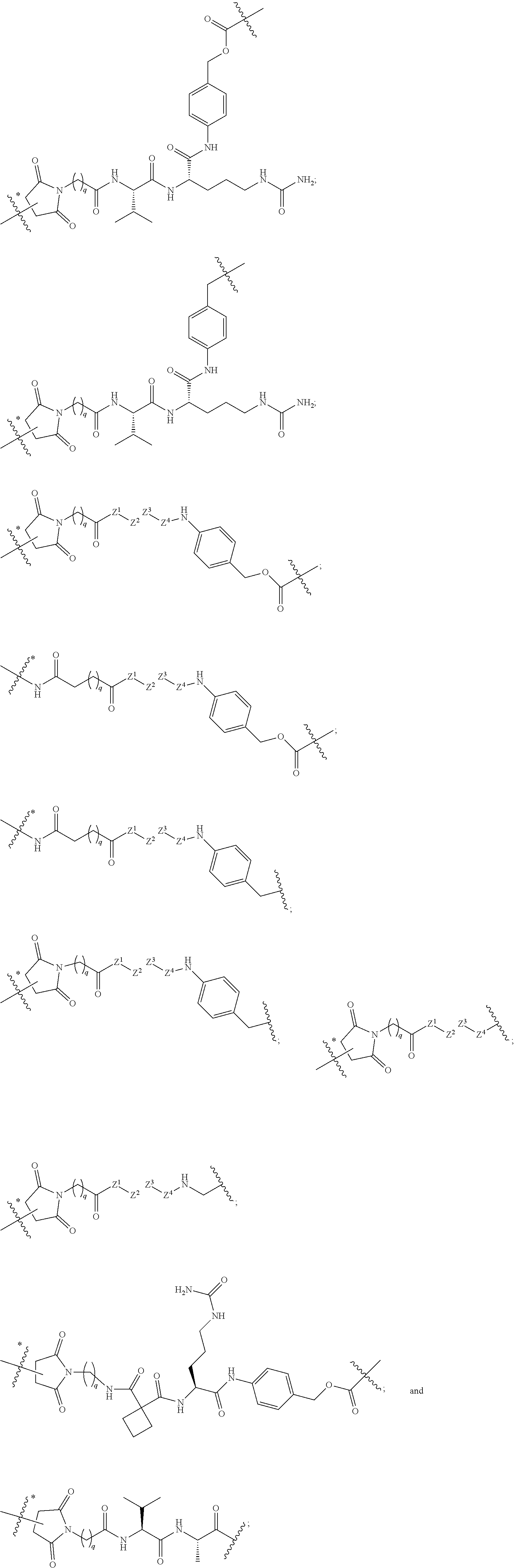

wherein:
   q is an integer from 2 to 10;
   Z¹, Z², Z³, and Z⁴ are each independently absent or a naturally-occurring amino acid residue in the L- or D-configuration, provided that at least two of Z¹, Z², Z³, and Z⁴ are amino acid residues;
   [symbol] is the point of attachment to the payload; and
   [symbol] is the point of attachment to the binding moiety.

In certain aspects, Z¹, Z², Z³, and Z⁴ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, and Z⁴ are amino acid residues.

In some aspects, Z¹ is absent or glycine; Z² is absent or selected from L-glutamine, D-glutamine, L-glutamic acid, D-glutamic acid, L-aspartic acid, D-aspartic acid, L-alanine, D-alanine, and glycine; Z³ is selected from L-valine, D-valine, L-alanine, D-alanine, L-phenylalanine, D-phenylalanine, and glycine; and Z⁴ is selected from L-alanine, D-alanine, L-citrulline, D-citrulline, L-asparagine, D-asparagine, L-lysine, D-lysine, L-phenylalamine, D-phenylalanine, and glycine.

In some aspects, L is selected from

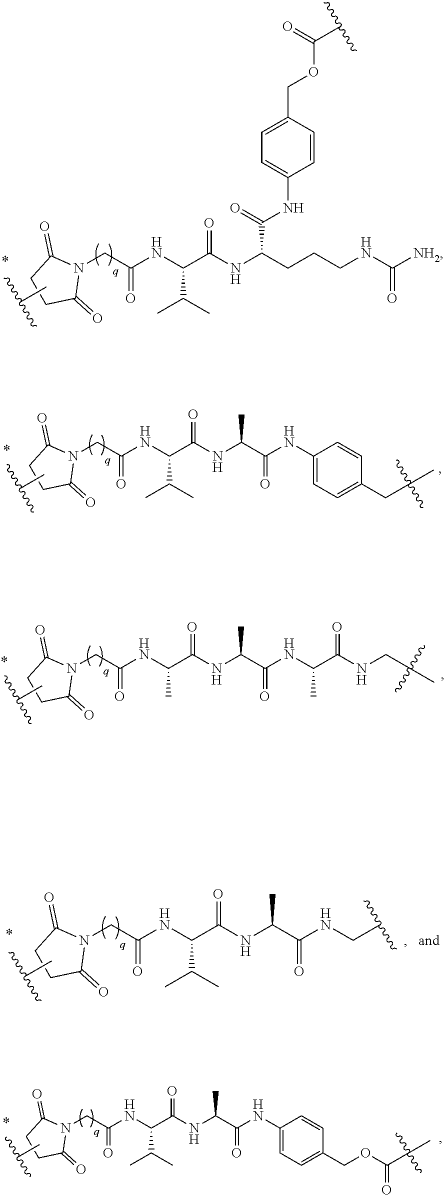

wherein:
   q is an integer from 2 to 10;
   [symbol] is the point of attachment to the payload; and
   [symbol] is the point of attachment to the binding moiety.

In some aspects, the linker is bioreducible. Bioreducible linkers take advantage of the difference in reduction potential in the intracellular compartment versus plasma. Reduced glutathione presented in tumor cells' cytoplasma is up to 1000-fold higher than that present in normal cells' cytoplasma, and the tumor cells also contain enzymes which can contribute to reduction in cellular compartments. The linkers keep conjugates intact during systemic circulation, and are selectively cleaved by the high intracellular concentration of glutathione, releasing the active drugs at the tumor sites from the non-toxic prodrugs.

In some aspects, L is a bioreducible linker selected from:

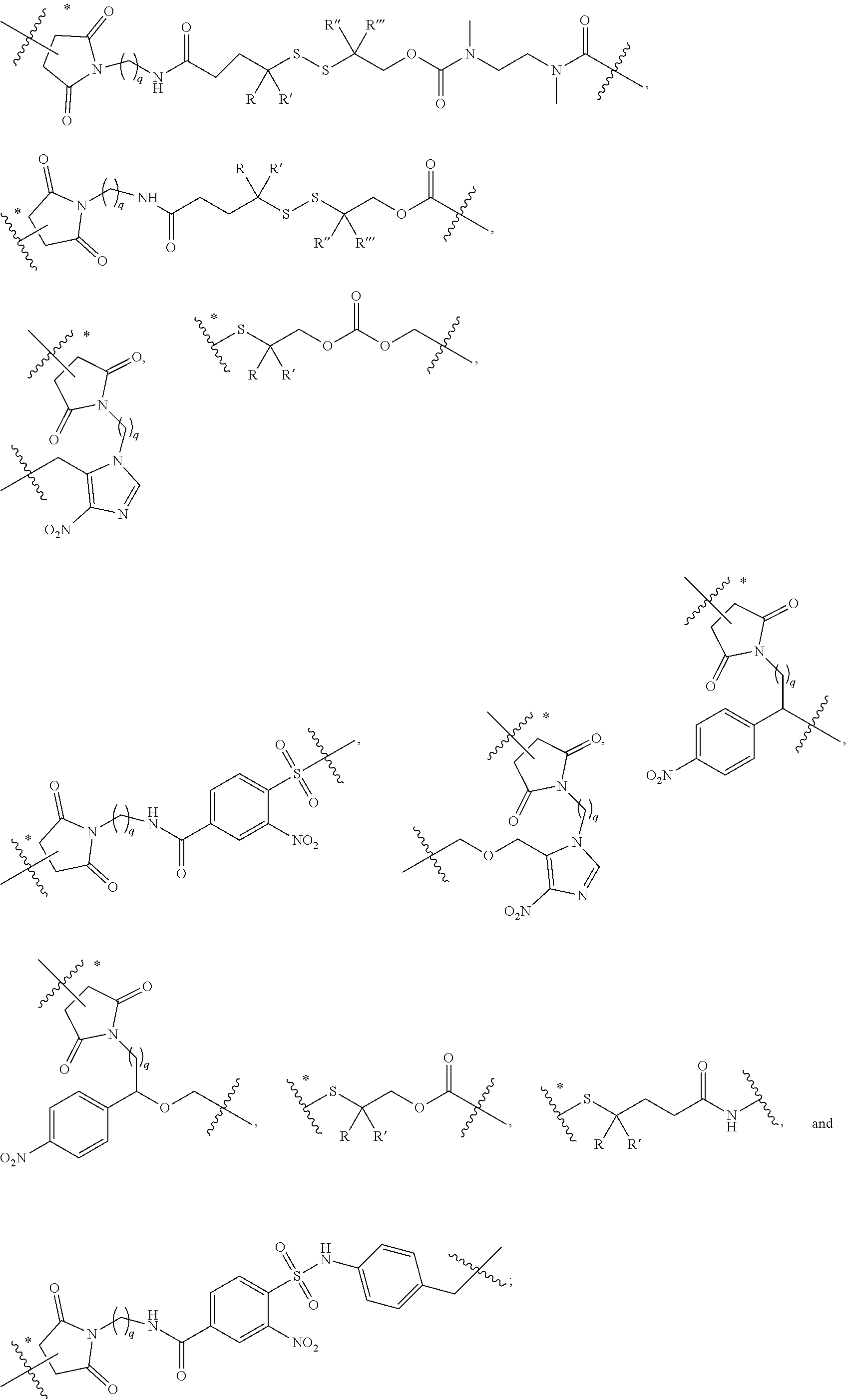

wherein:
   q is an integer from 2 to 10;
   R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring;
   [symbol] is the point of attachment to the payload; and
   [symbol] is the point of attachment to the binding moiety.

In some aspects, L is selected from

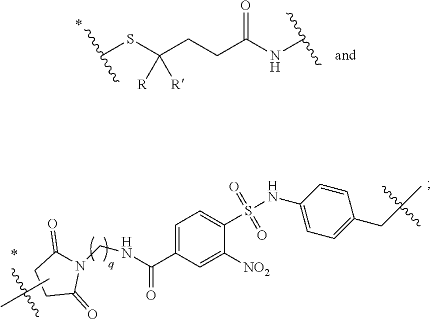

wherein:
   q is an integer from 2 to 10;
   R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring;
   [symbol] is the point of attachment to the payload; and
   [symbol] is the point of attachment to the binding moiety.

In certain aspects, L is

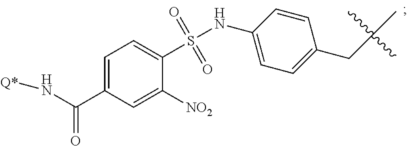

wherein Q* is a group that can be attached to a binding moiety, and [symbol] is the point of attachment to the payload.

In some aspects, the present disclosure provides a linker of formula L**

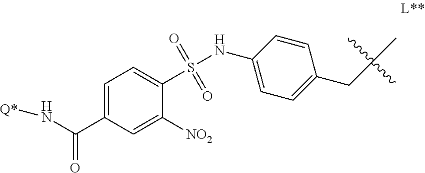

wherein Q* is a group that can be attached to a binding moiety, and [symbol] is the point of attachment to a payload. In some aspects, the linker can be used to connect any payload to any binding moiety.

In certain aspects, the linker is acid cleavable. Acid-cleavable linkers are specifically designed to remain stable at the neutral pH of blood circulation, but undergo hydrolysis and release the cytotoxic drug in the acidic environment of the cellular compartments.

In some aspects, L is an acid cleavable linker selected from

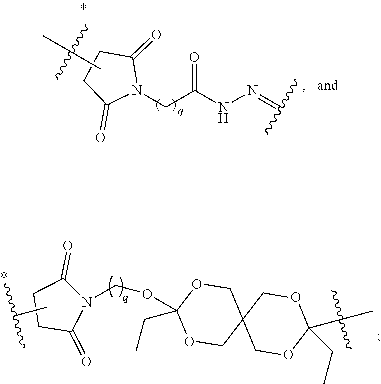

wherein:
   q is an integer from 2 to 10;
   [symbol] is the point of attachment to the payload; and
   [symbol] is the point of attachment to the binding moiety.

In certain aspects, L is wherein L is a click-to-release linker, where release of the payload is chemically triggered by a tetrazine or related compound.

In some aspects, L is a click-to-release linker selected from

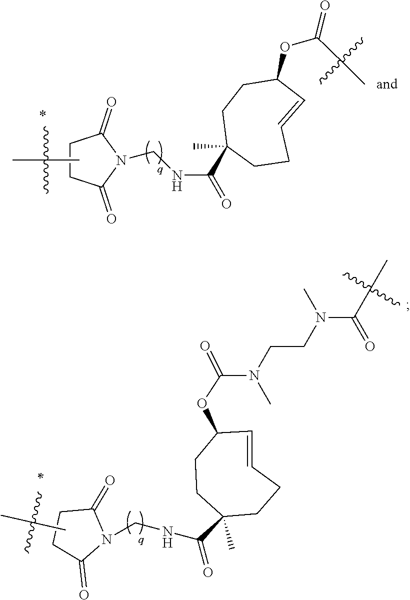

wherein:
   q is an integer from 2 to 10;
   [symbol] is the point of attachment to the payload; and
   [symbol] is the point of attachment to the binding moiety.

In certain aspects, L is a pyrophosphatase cleavable linker.

In some aspects, L is a pyrophosphatase cleavable linker which is:

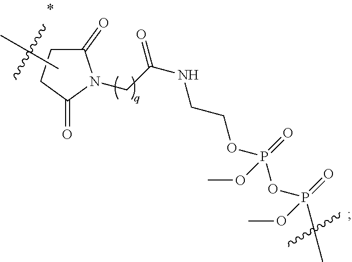

wherein:
   q is an integer from 2 to 10;
   [symbol] is the point of attachment to the payload; and
   [symbol] is the point of attachment to the binding moiety.

In certain aspects, L is a beta-glucoronidase cleavable linker.

In some aspects, L is a beta-glucoronidase cleavable linker selected from:

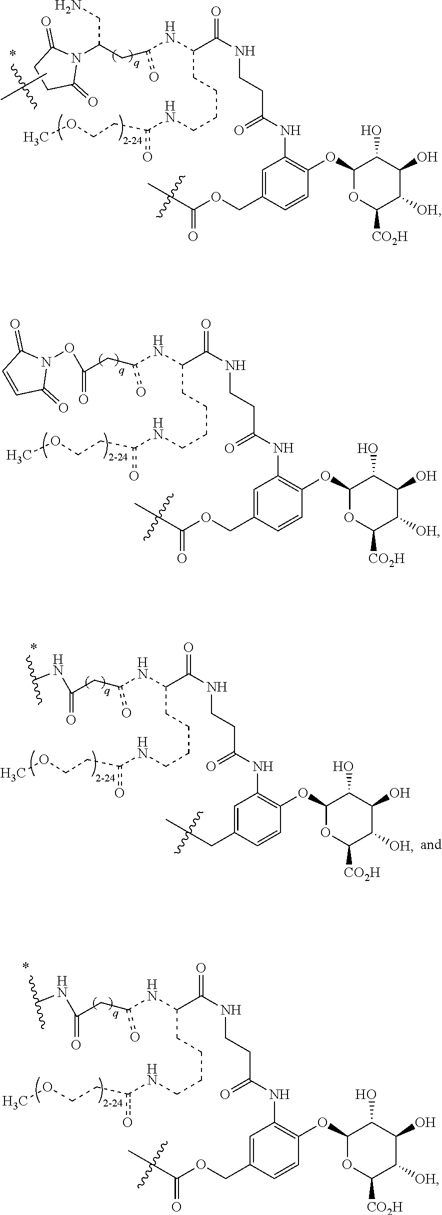

wherein:
   q is an integer from 2 to 10;
   [symbol] is absent or a bond;
   [symbol] is the point of attachment to the payload; and
   [symbol] is the point of attachment to the binding moiety.

In certain aspects, L is

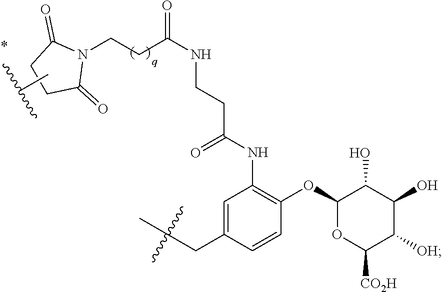

wherein:
   q is an integer from 2 to 10;
   [symbol] is absent or a bond;
   [symbol] is the point of attachment to the payload; and
   [symbol] is the point of attachment to the binding moiety.

In some aspects, the point of attachment to the binding moiety is a cysteine, lysine, tyrosine, or glutamine in the binding moiety. In some aspects, the point of attachment to the binding moiety is a cysteine. In some aspects, the point of attachment to the binding moiety is a lysine. In some aspects, the point of attachment to the binding moiety is a tyrosine. In some aspects, the point of attachment to the binding moiety is a glutamine (e.g., the glutamine at heavy chain position 295 of an antibody or antigen binding portion thereof according to EU numbering).

The cysteine or lysine can be an engineered (i.e., not endogenous to the binding moiety) cysteine or lysine, e.g., for site-specific conjugation. Site-specific conjugation refers to attachment through unique and defined sites on the binding moiety (e.g., antibody or antigen binding portion thereof). Site-specific conjugation is discussed, for example, in Zhou, Qun. “Site-Specific Antibody Conjugation for ADC and Beyond.” Biomedicines vol. 5 (4), 64. 9 Nov. 2017, doi:10.3390/biomedicines5040064, which is herein incorporated by reference in its entirety. In some aspects, the engineered cysteine is at heavy chain position S239 and/or K334 of an antibody or antigen binding portion thereof according to EU numbering.

The cysteine or lysine, which is the point of attachment can be a cysteine or lysine that is endogenous to the binding moiety.

### III.C. Binding Moieties

As demonstrated herein, a binding moiety (Bm) can deliver a payload, e.g., an activator of an effector T cell, to an effector T cell.

The term “binding moiety,” as used herein, refers to any molecule that recognizes and binds to a target. A binding moiety capable of specifically bind a target on the surface of an effector T cell refers to any molecule that recognizes and binds to a target that is present on an effector T cell. The target can also be present on the surface of other cells (e.g., other T cells).

In some aspects, the target is expressed on T cells (including effector T cells) but is not expressed on other cells that are not T cells. In some aspects, the expression of the target on effector T cells is enhanced as compared to the expression of the target on other T cells. For example, in some aspects, the expression of the target on effector T cells is at least 5-fold greater on effector T cells than on regulatory T cells. For example, in some aspects, the expression of the target on effector T cells is at least 10-fold greater on effector T cells than on regulatory T cells. For example, in some aspects, the expression of the target on effector T cells is at least 20-fold greater on effector T cells than on regulatory T cells.

In some aspects, the target is expressed on regulatory T cells and effector T cells. In some aspects where the target is expressed on regulatory T cells and effector T cells, the expression of the target on regulatory T cells is greater than the expression on effector T cells. In some aspects where the target is expressed on regulatory T cells and effector T cells, the expression of the target on regulatory T cells is greater than the expression on helper T cells. In some aspects where the target is expressed on regulatory T cells and effector T cells, the expression of the target on regulatory T cells is greater than the expression on effector T cells and greater than the expression on helper T cells.

In some aspects, the target is a protein, glycoprotein, lipid, glycolipid, or carbohydrate on the surface of an effector T cell. In some aspects, the target is a protein or glycoprotein.

The binding moiety, in addition to targeting the compound(s) to a specific cell, tissue, or location, can also have a therapeutic effect (e.g., proliferative effect or migratory effect) on an effector T cell. Following specific binding of the binding moiety to a target on the surface of an effector T cell, the effector T cell can internalize a conjugate comprising the binding moiety, a payload attached to the binding moiety, and/or the target.

In certain aspects, the binding moiety can comprise or can be engineered to comprise at least one chemically reactive group such as a carboxylic acid, amine, thiol, or chemically reactive amino acid moiety or side chain.

In some aspects, a binding moiety (Bm) can be a peptide or a protein that binds to a cell surface receptor or antigen.

In certain aspects, group “Bm” can be an antibody or an antigen-binding fragment thereof. In some aspects, the antibody or antigen-binding fragment thereof is a monoclonal antibody or antigen-binding fragment thereof. Exemplary sequences of antibodies or antigen-binding fragments thereof are provided herein in Tables 2-4. An antibody or antigen-binding fragment thereof can be of any immunoglobulin class, including IgG, IgM, IgE, IgA, and IgD and any subclass thereof. In some aspects, an antibody or antigen-binding fragment thereof is an IgG antibody. In some aspects, an antibody or antigen-binding fragment thereof is an IgG1 or an IgG4 antibody or antigen-binding fragment thereof. In some aspects, an antibody or antigen-binding fragment thereof is an IgG1 antibody or antigen-binding fragment thereof. In some aspects, an antibody or antigen-binding fragment thereof is an IgG4 antibody or antigen-binding fragment thereof.

Useful monoclonal antibodies or antigen-binding fragments thereof include, but are not limited to, human monoclonal antibodies or antigen-binding fragments thereof, humanized monoclonal antibodies or antigen-binding fragments thereof, or chimeric human-mouse (or other species) monoclonal antibodies or antigen-binding fragments thereof. Human monoclonal antibodies or antigen-binding fragments thereof can be made by any of numerous techniques known in the art.

Useful antigen-binding fragments of antibodies include F(ab′)2 fragments, which contain the variable region, the light chain constant region and the CH1 domain of the heavy chain, and can be produced by pepsin digestion of the antibody molecule, and Fab fragments, which can be generated by reducing the disulfide bridges of the F(ab′)2 fragments. Other useful antibodies and antigen-binding fragments thereof are heavy chain and light chain dimers, or any minimal fragment thereof such as Fvs or single chain antibodies (SCAs), or any other molecule with the same specificity as the antibody.

Additionally, recombinant antibodies and antigen-binding fragments thereof, such as chimeric and humanized monoclonal antibodies and antigen-binding fragments thereof, comprising both human and non-human portions, which can be made using standard recombinant DNA techniques, are useful antibodies and antigen-binding fragments thereof.

Completely human antibodies or antigen-binding fragments thereof can be produced using transgenic mice that are incapable of expressing endogenous immunoglobulin heavy and light chains genes, but which can express human heavy and light chain genes. The transgenic mice are immunized in the normal fashion with a selected antigen, e.g., all or a portion of a polypeptide of the disclosure. Monoclonal antibodies or antigen-binding fragments thereof directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies or antigen-binding fragments thereof. For an overview of this technology for producing human antibodies or antigen-binding fragments thereof, see Lonberg and Huszar (1995, Int. Rev. Immunol. 13:65-93). Other human antibodies can be obtained commercially from, for example, Abgenix, Inc. (Freemont, Calif.) and Genpharm (San Jose, Calif.).

Completely human antibodies or antigen-binding fragments thereof that recognize a selected epitope can be generated using a technique referred to as “guided selection.” In this approach a selected non-human monoclonal antibody or antigen-binding fragment thereof, e.g., a mouse antibody or antigen-binding fragment thereof, is used to guide the selection of a completely human antibody or antigen-binding fragment thereof recognizing the same epitope. Human antibodies or antigen-binding fragments thereof can also be produced using various techniques known in the art, including phage display libraries.

The antibody or antigen-binding fragment thereof can be a fusion protein of an antibody or antigen-binding fragment thereof, for example in which the antibody or fragment is fused via a covalent bond (e.g., a peptide bond), at either the N-terminus or the C-terminus to an amino acid sequence of another protein (or portion thereof, such as at least 10, 20 or 50 amino acid portion of the protein) that is not the antibody or fragment. The antibody or antigen-binding fragment thereof can be covalently linked to the other protein at the N-terminus of the constant domain.

Antibodies and antigen-binding fragments thereof include analogs and derivatives that are either modified, i.e., by the covalent attachment of any type of molecule as long as such covalent attachment permits the antibody or antigen-binding fragment to retain its antigen binding immunospecificity. For example, but not by way of limitation, the derivatives and analogs of the antibodies and antigen-binding fragments thereof include those that have been further modified, e.g., by glycosylation, acetylation, pegylation, phosphorylation, amidation, derivatization by known protecting/blocking groups, proteolytic cleavage, linkage to a cellular antibody unit or other protein, etc. Any of numerous chemical modifications can be carried out by known techniques, including, but not limited to specific chemical cleavage, acetylation, formylation, metabolic synthesis in the presence of tunicamycin, etc. Additionally, the analog or derivative can contain one or more unnatural amino acids.

The antibodies or antigen-binding fragments thereof in the conjugates can include antibodies or antigen-binding fragments thereof having modifications (e.g., substitutions, deletions or additions) in amino acid residues that interact with Fc receptors. In particular, antibodies or antigen-binding fragments thereof include antibodies or antigen-binding fragments thereof having modifications in amino acid residues identified as involved in the interaction between the anti-Fc domain and the FcRn receptor. Antibodies or antigen-binding fragments thereof capable of specifically binding to a target on the surface of an effector T cell can be obtained commercially or produced by any method known to one of skill in the art such as, e.g., chemical synthesis or recombinant expression techniques. The nucleotide sequence encoding antibodies or antigen-binding fragments thereof capable of specifically binding to a target on the surface of an effector T cell can be obtained, e.g., from the GenBank database or a database like it, the literature publications, or by routine cloning and sequencing.

In certain aspects, the antibody or antigen-binding fragment thereof of the conjugates can be a monoclonal antibody or antigen-binding fragment thereof, e.g. a murine monoclonal antibody or antigen-binding fragment thereof, a chimeric antibody or antigen-binding fragment thereof, or a humanized antibody or antigen-binding fragment thereof. In some aspects, the antibody or antigen-binding fragment thereof can be, e.g. a Fab fragment.

As provided herein, binding moieties (e.g., antibodies or antigen-binding fragments thereof) that bind to targets (antigens) on the surface of effector T cells can be conjugated to a payload. By way of example, such a binding moiety (Bm), e.g., antibody or antigen-binding fragment thereof, can bind to PD1, CD25 (IL2RA), or CD7.

In some aspects, a binding moiety (Bm), e.g., an antibody or antigen-binding fragment thereof, can specifically bind CD25 (also known as interleukin-2 receptor subunit alpha (IL2RA). Such binding moieties are also referred to herein as “anti-IL2RA binding moieties,” “IL2RA binding moieties,” “anti-CD25 binding moieties,” or “CD25 binding moieties,” e.g., anti-IL2RA antibodies or antigen-binding fragments thereof, IL2RA antibodies or antigen-binding fragments thereof, anti-CD25 antibodies or antigen-binding fragments thereof or CD25 antibodies or antigen-binding fragments thereof. Exemplary sequences of anti-TL2RA or anti-CD25 antibodies or antigen-binding fragments thereof are provided in Table 2. In some aspects, an anti-IL2RA or anti-CD25 antibody or antigen-binding fragment thereof comprises the heavy and light chain CDR sequences (e.g., Kabat, Chothia, or AbM-defined CDRs) of SEQ ID NOs:4 and 5, respectively (e.g., amino acids 31-35, 50-66, and 99-111 of SEQ ID NO:4 and amino acids 24-34, 50-56, and 89-97 of SEQ ID NO:5). In some aspects, an anti-IL2RA or anti-CD25 antibody or antigen-binding fragment thereof comprises a variable heavy chain comprising the amino acid sequence of SEQ ID NO:4 and/or a variable light chain comprising the amino acid sequence of SEQ ID NO:5. In some aspects, an anti-IL2RA or anti-CD25 antibody or antigen-binding fragment thereof comprises the heavy and light chain CDR sequences (e.g., Kabat, Chothia, or AbM-defined CDRs) of SEQ ID NOs:6 and 7, respectively (e.g,. amino acids 31-35, 50-65, and 98-108 of SEQ ID NO:6 and amino acids 24-33, 49-55, and 88-96 of SEQ ID NO:7). In some aspects, an anti-TL2RA or anti-CD25 antibody or antigen-binding fragment thereof comprises a variable heavy chain comprising the amino acid sequence of SEQ TD NO:6 and/or a variable light chain comprising the amino acid sequence of SEQ ID NO:7. In some aspects, an anti-IL2RA or anti-CD25 antibody or antigen-binding fragment thereof comprises the heavy and light chain CDR sequences (e.g., Kabat, Chothia, or AbM-defined CDRs) of SEQ TD NOs:8 and 9, respectively (e.g., amino acids 31-35, 50-65, and 98-108 of SEQ ID NO:8 and amino acids 24-33, 49-55, and 88-96 of SEQ ID NO:9). In some aspects, an anti-IL2RA or ant-CD25 antibody or antigen-binding fragment thereof comprises a variable heavy chain comprising the amino acid sequence of SEQ ID NO:8 and/or a variable light chain comprising the amino acid sequence of SEQ ID NO:9. In some aspects, an anti-TLRA or ant-CD25 antibody or antigen-binding fragment thereof comprises the heavy and light chain CDR sequences (e.g., Kabat, Chothia, or AbM-defined CDRs) of SEQ TD NOs:16 and 17, respectively (e.g., amino acids 31-35, 50-66, and 99-103 of SEQ ID NO: 16 and amino acids 24-39, 55-61, and 94-102 of SEQ ID NO:17). In some aspects, an anti-IL2RA or ant-CD25 antibody or antigen-binding fragment thereof comprises a variable heavy chain comprising the amino acid sequence of SEQ TD NO: 16 and/or a variable light chain comprising the amino acid sequence of SEQ ID NO: 17.

TABLE 2
Exemplary IL2RA or CD25 Antibody or Antigen Binding Fragment Thereof Sequences

| | VH (CDR sequences underlined) | VL (CDR sequences underlined) |
|---|---|---|
| Antibody | QVQLVQSGAEVKKPGSSVKVSCK | DIQMTQSPSTLSASVGDRVTITCR |
| ‘A’ | ASGGTFSSLAISWVRQAPGQGLEW | ASQSISSWLAWYQQKPGKAPKLL |
| a686 | MGGIIPIFGTANYAQKFQGRVTITA | IYKASSLESGVPSRFSGSGSGTEF |
| (anti- | DESTSTAYMELSSLRSEDTAVYYC | TLTISSLQPDDFATYYCQQYNIYP |
| human | ARGGSVSGTLVDFDIWGQGTMVT | ITFGGGTKVEIK (SEQ ID NO: 5) |
| CD25) | VSS (SEQ ID NO: 4) | |
| Antibody | QVQLKESGPGLVAPSQSLSITCTVS | QIVLSQSPAILSASPGEKVTMTCR |
| ‘B’ | GFSLTSYGIQWVRQPPGKGLEWLG | ASSSVSYMHWYQQKPGSSPKPWI |
| MA251 | VIWAGGSTNYNSALMSRLSISKDN | FATSNLASGVPARFSGSGSGTSYS |
| (anti- | SKSQVFLKMNSLQTDDTAMYYCA | LTINRVEAEDADTYYCQQWSSNP |
| human | RAYGYDGSWLAYWGQGTLVTVSS | PTFGGGTKLEIK (SEQ ID NO: 7) |
| CD25) | (SEQ ID NO: 6) | |
| Antibody | QVQLQESGPGLVKPSETLSLTCTVS | DIQLTQSPSSLSASVGDRVTITCR |
| ‘C’ | GFSLTSYGIQWIRQPPGKGLEWLG | ASSSVSYMHWYQQKPGKAPKPW |
| Human- | VIWAGGSTNYNSALMSRLTISKDN | IFATSNLASGVPSRFSGSGSGTDY |
| ized | SKNQVSLKLSSVTAADTAVYYCA | TLTISSLQPEDFATYYCQQWSSNP |
| MA251 | RAYGYDGSWLAYWGQGTMVTVS | PTFGGGTKVEIK (SEQ ID NO: 9) |
| (anti- | S (SEQ ID NO: 8) | |
| human | | |
| CD25) | | |
| Antibody | EVQLQQSGAALVKPGASVKMSCK | DVVLTQTPPTLSATIGQSVSISCRS |
| ‘D’ | ASGYSFPDSWVTWVKQSHGKSLE | SQSLLHSNGNTYLNWLLQRPGQP |
| 7D4 (anti- | WIGDIFPNSGATNFNEKFKGKATL | PQLLIYLASRLESGVPNRFSGSGS |
| mouse | TVDKSTSTAYMELSRLTSEDSAIYY | GTDFTLKISGVEAEDLGVYYCVQ |
| CD25) | CTRLDYGYWGQGVMVTVSS (SEQ | SSHFPNTFGVGTKLEIK (SEQ ID |
| | ID NO: 16) | NO: 17) |

In some aspects, a binding moiety (Bm), e.g., an antibody or antigen-binding fragment thereof, can specifically bind PD1. Such binding moieties are also referred to herein as “anti-PD1 binding moieties” or “PD1 binding moieties,” e.g., anti-PD1 antibodies or antigen-binding fragments thereof or PD1 antibodies or antigen-binding fragments thereof. Exemplary sequences of anti-PD1 antibodies or antigen-binding fragments thereof are provided in Table 3. In some aspects, an anti-PD1 antibody or antigen-binding fragment thereof comprises the heavy and light chain CDR sequences (e.g., Kabat, Chothia, or AbM-defined CDRs) of SEQ ID NOs:10 and 11, respectively (e.g., amino acids 31-35, 50-66, and 99-109 of SEQ ID NO:10 and amino acids 24-38, 54-60, and 93-101 of SEQ ID NO:11). In some aspects, an anti-PD1 antibody or antigen-binding fragment thereof comprises a variable heavy chain comprising the amino acid sequence of SEQ ID NO:10 and/or a variable light chain comprising the amino acid sequence of SEQ ID NO: 11. In some aspects, an anti-PD1 antibody or antigen-binding fragment thereof comprises the heavy and light chain CDR sequences (e.g., Kabat, Chothia, or AbM-defined CDRs) of SEQ ID NOs:12 and 13, respectively (e.g., amino acids 31-35, 50-66, and 99-102 of SEQ ID NO:12 and amino acids 24-34, 50-56, and 89-97 of SEQ ID NO:13). In some aspects, an anti-PD1 antibody or antigen-binding fragment thereof comprises a variable heavy chain comprising the amino acid sequence of SEQ ID NO:12 and/or a variable light chain comprising the amino acid sequence of SEQ ID NO:13. In some aspects, an anti-PD1 antibody or antigen-binding fragment thereof comprises the heavy and light chain CDR sequences (e.g., Kabat, Chothia, or AbM-defined CDRs) of SEQ ID NOs:18 and 19, respectively (e.g., amino acids 31-35, 50-68, and 101-110 of SEQ ID NO:18 and amino acids 24-33, 49-55, and 88-100 of SEQ ID NO:19). In some aspects, an anti-PD1 antibody or antigen-binding fragment thereof comprises a variable heavy chain comprising the amino acid sequence of SEQ ID NO:18 and/or a variable light chain comprising the amino acid sequence of SEQ ID NO:19. In some aspects, an anti-PD1 antibody or antigen-binding fragment thereof comprises a heavy chain comprising the amino acid sequence of SEQ ID NO:26 and/or a light chain comprising the amino acid sequence of SEQ ID NO:27.

TABLE 3
Exemplary Anti-PD1 Antibody or Antigen Binding Fragment Thereof Sequences

| | VH (CDR sequences underlined) | VL (CDR sequences underlined) |
|---|---|---|
| Antibody | QVQLVQSGVEVKKPGASVKVSCK | EIVLTQSPATLSLSPGERATLSCR |
| 'E’ | ASGYTFTNYYMYWVRQAPGQGLE | ASKGVSTSGYSYLHWYQQKPGQ |
| Pembro- | WMGGINPSNGGTNFNEKFKNRVT | APRLLIYLASYLESGVPARFSGSG |
| lizumab | LTTDSSTTTAYMELKSLQFDDTAV | SGTDFTLTISSLEPEDFAVYYCQH |
| (anti- | YYCARRDYRFDMGFDYWGQGTT | SRDLPLTFGGGTKVEIK (SEQ ID |
| human | VTVSS (SEQ ID NO: 10) | NO: 11) |
| PD1) | | |
| Antibody | QVQLVESGGGVVQPGRSLRLDCK | EIVLTQSPATLSLSPGERATLSCR |
| ‘F’ | ASGITFSNSGMHWVRQAPGKGLE | ASQSVSSYLAWYQQKPGQAPRL |
| Nivo- | WVAVIWYDGSKRYYADSVKGRFT | LIYDASNRATGIPARFSGSGSGTD |
| lumab | ISRDNSKNTLFLQMNSLRAEDTAV | FTLTISSLEPEDFAVYYCQQSSNW |
| (anti- | YYCATNDDYWGQGTLVTVSS | PRTFGQGTKVEIK (SEQ ID NO: 13) |
| human | (SEQ ID NO: 12) | |
| PD1) | | |
| Antibody | EVRLLESGGGLVKPEGSLKLSCVA | SYELTQPPSASVNVGETVKITCSG |
| ‘G’ | SGFTFSDYFMSWVRQAPGKGLEW | DQLPKYFADWFHQRSDQTILQVI |
| J43 (anti- | VAHIYTKSYNYATYYSGSVKGRFT | YDDNKRPSGIPERISGSSSGTTAT |
| mouse | ISRDDSRSMVYLQMNNLRTEDTAT | LTIRDVRAEDEGDYYCFSGYVDS |
| PD1) | YYCTRDGSGYPSLDFWGQGTQVT | DSKLYVFGSGTQLTVL (SEQ ID |
| | VSS (SEQ ID NO: 18) | NO: 19) |
| | Heavy Chain | Light Chain |
| Antibody | QVQLVQSGVEVKKPGASVKVSCK | EIVLTQSPATLSLSPGERATLSCR |
| ‘I’ | ASGYTFTNYYMYWVRQAPGQGLE | ASKGVSTSGYSYLHWYQQKPGQ |
| | WMGGINPSNGGTNFNEKFKNRVT | APRLLIYLASYLESGVPARFSGSG |
| | LTTDSSTTTAYMELKSLQFDDTAV | SGTDFTLTISSLEPEDFAVYYCQH |
| | YYCARRDYRFDMGFDYWGQGTT | SRDLPLTFGGGTKVEIKRTVAAPS |
| | VTVSSASTKGPSVFPLAPSSKSTSG | VFIFPPSDEQLKSGTASVVCLLNN |
| | GTAALGCLVKDYFPEPVTVSWNS | FYPREAKVQWKVDNALQSGNSQ |
| | GALTSGVHTFPAVLQSSGLYSLSSV | ESVTEQDSKDSTYSLSSTLTLSKA |
| | VTVPSSSLGTQTYICNVNHKPSNTK | DYEKHKVYACEVTHQGLSSPVT |
| | VDKKVEPKSCDKTHTCPPCPAPEA | KSFNRGEC (SEQ ID NO: 27) |
| | AGGPSVFLFPPKPKDTLMISRTPEV | |
| | TCVVVDVSHEDPEVKFNWYVDGV | |
| | EVHNAKTKPREEQYASTYRVVSVL | |
| | TVLHQDWLNGKEYKCKVSNKALP | |
| | APIEKTISKAKGQPREPQVYTLPPS | |
| | RDELTKNQVSLTCLVKGFYPSDIA | |
| | VEWESNGQPENNYKTTPPVLDSDG | |
| | SFFLYSKLTVDKSRWQQGNVFSCS | |
| | VMHEALHNHYTQKSLSLSPG (SEQ | |
| | ID NO: 26) | |

In some aspects, a binding moiety (Bm), e.g., an antibody or antigen-binding fragment thereof, can specifically bind CD7. Such binding moieties are also referred to herein as “anti-CD7 binding moieties” or “CD7 binding moieties,” e.g., anti-CD7 antibodies or antigen-binding fragments thereof or CD7 antibodies or antigen-binding fragments thereof. Exemplary sequences of anti-CD7 antibodies or antigen-binding fragments thereof are provided in Table 4. In some aspects, an anti-CD7 antibody or antigen-binding fragment thereof comprises the heavy and light chain CDR sequences (e.g., Kabat, Chothia, or AbM-defined CDRs) of SEQ ID NOs:14 and 15, respectively (e.g., amino acids 31-35, 50-66, and 99-112 of SEQ ID NO:14 and amino acids 23-36, 52-58, and 91-99 of SEQ ID NO:15). In some aspects, an anti-CD7 antibody or antigen-binding fragment thereof comprises a variable heavy chain comprising the amino acid sequence of SEQ ID NO:14 and/or a variable light chain comprising the amino acid sequence of SEQ ID NO:15.

TABLE 4
Exemplary Anti-CD7 Antibody or Antigen Binding Fragment Thereof Sequences

| | VH (CDR sequences underlined) | VL (CDR sequences underlined) |
|---|---|---|
| Antibody | QIQLVQSGPELKKPGETVKISCKAS | QAVVTQESALTTSPGETVTLTCR |
| ‘H’ | GYTFTNYGMNWVKQAPGKGLMW | SSTGAVTTSNYANWVQEKPDHL |
| WT1 CS | LGWINTYTGEPTYADDFKGRFAFS | FTGLIGGTNNRAPGVPARFSGSLI |
| (anti- | LETSASTAYLQINNLKNEDTATYF | GDKAALTITGAQTEDEAIYFCAL |
| human | CARWAYFYGSSPYFFDYWGQGTT | WSSNHLVFGGGTKLTVL (SEQ ID |
| CD7) | LTVSS (SEQ ID NO: 14) | NO: 15) |

In some aspects, an antibody or antigen binding portion thereof comprises a constant region. A linker can be attached to an amino acid in the constant region. In some aspects, an antibody or antigen binding portion thereof comprises a CH1 domain. A linker can be attached to an amino acid in a CH1 domain. In some aspects, an antibody or antigen binding portion thereof comprises a CH2 domain. A linker can be attached to an amino acid in a CH2 domain. In some aspects, an antibody or antigen binding portion thereof comprises a CH3 domain. A linker can be attached to an amino acid in a CH3 domain. In some aspects, an antibody or antigen binding portion thereof comprises a CL domain. A linker can be attached to an amino acid in a CL domain.

In some aspects, a constant region, a CH1 domain, a CH2 domain, a CH3 domain, or a CL domain is an engineered constant region, CH1 domain, CH2 domain, CH3 domain or a CL domain.

In some aspects, an antibody or antigen binding portion thereof comprises a heavy chain constant region, e.g., a human heavy chain constant region. A linker can be attached to an amino acid in a heavy chain constant region, e.g., a human heavy chain constant region. In some aspects, an antibody or antigen binding portion thereof comprises an IgG heavy chain constant region, e.g., a human IgG heavy chain constant region. A linker can be attached to an amino acid in an IgG heavy chain constant region, e.g., a human IgG heavy chain constant region. In some aspects, an antibody or antigen binding portion thereof comprises an IgG1 heavy chain constant region, e.g., a human IgG1 heavy chain constant region. A linker can be attached to an amino acid in an IgG1 heavy chain constant region, e.g., a human IgG1 heavy chain constant region. In some aspects, an antibody or antigen binding portion thereof comprises an IgG4 heavy chain constant region. A linker can be attached to an amino acid in an IgG4 heavy chain constant region, e.g., a human IgG4 heavy chain constant region.

In some aspects, an antibody or antigen binding portion thereof comprises a light chain constant region, e.g., a human light chain constant region. A linker can be attached to an amino acid in a light chain constant region, e.g., a human light chain constant region. In some aspects, an antibody or antigen binding portion thereof comprises a kappa light chain constant region, e.g., a human kappa light chain constant region. A linker can be attached to an amino acid in a kappa light chain constant region, e.g., a human kappa light chain constant region. In some aspects, an antibody or antigen binding portion thereof comprises a gamma light chain constant region, e.g., a human gamma light chain constant region. A linker can be attached to an amino acid in a gamma light chain constant region, e.g., a human gamma light chain constant region.

In some aspects, an antibody or antigen binding portion thereof comprises an engineered cysteine at heavy chain position S239 according to EU numbering. A linker can be attached to S239C. In some aspects, an antibody or antigen binding portion thereof comprises an engineered cysteine at heavy chain position K334 according to EU numbering. A linker can be attached to K334C.

Accordingly, an antibody or antigen binding portion thereof can comprise a heavy chain constant region of any of SEQ ID NOs:20-24.

IgG1 Heavy Chain Constant Region
(SEQ ID NO: 20)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKS
RWQQGNVFSCSVMHEALHNHYTQKSLSLSPG
IgG1 N297A Constant regions (CH1-Hinge-CH2-CH3)
(SEQ ID NO: 21)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQY
ASTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKS
RWQQGNVFSCSVMHEALHNHYTQKSLSLSPG
IgG4 S228P Constant regions (CH1-Hinge-CH2-CH3)
(SEQ ID NO: 22)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRW
QEGNVFSCSVMHEALHNHYTQKSLSLSLG
IgG1 Heavy Chain Constant Region S239C
(SEQ ID NO: 23)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PCVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKS
RWQQGNVFSCSVMHEALHNHYTQKSLSLSPG
IgG1 Heavy Chain Constant Region K334C
(SEQ ID NO: 24)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIECTISKAKGQPREPQVYTLPPSR
DELTKNQVSLTCLVKGFYPSDIAVEWESNGQPENNYKTTPPVLDSDGSFFLYSKLTVDKS
RWQQGNVFSCSVMHEALHNHYTQKSLSLSPG

In some aspects, an antibody or antigen binding portion thereof can comprise a heavy chain constant region of SEQ ID NO:28.

(SEQ ID NO: 28)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVV
DVSHEDPEVKFNWYVDGVEVHNAKTKPREEQYASTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPG

In some aspects, a linker can be attached to heavy chain Q295 of an antibody or antigen binding portion thereof according to EU numbering.

In the present disclosure, group “Bm” can be conjugated to more than one compound capable of activating an effector T cell. In some aspects, “Bm” can be conjugated to from 1 to 10 compounds. In some aspects, “Bm” can be conjugated to from 1 to 9 compounds. In some aspects, “Bm” can be conjugated to from 1 to 8 compounds. In some aspects, “Bm” can be conjugated to 1, 2, 3, 4, 5, 6, 7, 8, 9, or 10 compounds. In some aspects, “Bm” can be conjugated to 7 or 8 compounds. In some aspects, “Bm” is conjugated to 5 compounds. In some aspects, “Bm” is conjugated to 6 compounds s. In some aspects, “Bm” is conjugated to 7 compounds. In some aspects, “Bm” is conjugated to 8 compounds. In some aspects, “Bm” is conjugated to 9 compounds.

### IV. Compositions and Methods of Using

The conjugates and/or compounds described herein can be in the form of pharmaceutically or pharmaceutically acceptable salts. In some aspects, such salts are derived from inorganic or organic acids or bases.

Examples of suitable acid addition salts include acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2hydroxyethanesulfonate, lactate, maleate, methanesulfonate, 2naphthalenesulfonate, nicotinate, oxalate, pamoate, pectinate, persulfate, 3phenylpropionate, picrate, pivalate, propionate, succinate, tartrate, thiocyanate, tosylate and undecanoate.

Examples of suitable base addition salts include ammonium salts; alkali metal salts, such as sodium and potassium salts; alkaline earth metal salts, such as calcium and magnesium salts; salts with organic bases, such as dicyclohexylamine salts, N-methyl-D-glucamine; and salts with amino acids such as arginine, lysine, and the like.

For example, Berge lists the following FDA-approved commercially marketed salts: anions acetate, besylate (benzenesulfonate), benzoate, bicarbonate, bitartrate, bromide, calcium edetate (ethylenediaminetetraacetate), camsylate (camphorsulfonate), carbonate, chloride, citrate, dihydrochloride, edetate (ethylenediaminetetraacetate), edisylate (1,2ethanedisulfonate), estolate (lauryl sulfate), esylate (ethanesulfonate), fumarate, gluceptate (glucoheptonate), gluconate, glutamate, glycollylarsanilate (glycollamidophenylarsonate), hexylresorcinate, hydrabamine (N,N′di(dehydroabietyl)ethylenediamine), hydrobromide, hydrochloride, hydroxynaphthoate, iodide, isethionate (2hydroxyethanesulfonate), lactate, lactobionate, malate, maleate, mandelate, mesylate (methanesulfonate), methylbromide, methylnitrate, methylsulfate, mucate, napsylate (2-naphthalenesulfonate), nitrate, pamoate (embonate), pantothenate, phosphate/diphosphate, polygalacturonate, salicylate, stearate, subacetate, succinate, sulfate, tannate, tartrate, teoclate (8-chlorotheophyllinate) and triethiodide; organic cations benzathine (N,N′dibenzylethylenediamine), chloroprocaine, choline, diethanolamine, ethylenediamine, meglumine (Nmethylglucamine) and procaine; and metallic cations aluminum, calcium, lithium, magnesium, potassium, sodium and zinc.

Berge additionally lists the following non-FDA-approved commercially marketed (outside the United States) salts: anions adipate, alginate, aminosalicylate, anhydromethylenecitrate, arecoline, aspartate, bisulfate, butylbromide, camphorate, digluconate, dihydrobromide, disuccinate, glycerophosphate, hemisulfate, hydrofluoride, hydroiodide, methylenebis(salicylate), napadisylate (1,5-naphthalenedisulfonate), oxalate, pectinate, persulfate, phenylethylbarbiturate, picrate, propionate, thiocyanate, tosylate and undecanoate; organic cations benethamine (N-benzylphenethylamine), clemizole (1-p-chlorobenzyl-2-pyrrolildine-1′-ylmethylbenzimidazole), diethylamine, piperazine and tromethamine (tris(hydroxymethyl)aminomethane); and metallic cations barium and bismuth.

Pharmaceutical compositions comprising the conjugates described herein can also comprise suitable carriers, excipients, and auxiliaries that may differ depending on the mode of administration.

In some aspects, the pharmaceutical compositions can be formulated as a suitable parenteral dosage form. Said formulations can be prepared by various methods known in the art. The pharmaceutical compositions can be administered directly into the bloodstream. Suitable means for parenteral administration include intravenous administration.

Parenteral compositions are typically aqueous solutions which may contain excipients such as salts, carbohydrates and buffering agents. However, the composition may also be formulated a sterile non-aqueous solution or as a dried form to be used in conjunction with a suitable vehicle such as sterile pyrogen-free water.

The preparation of parenteral compositions under sterile conditions, for example, by lyophilization, can be readily accomplished using standard techniques known well to those of skill in the art.

The parenteral formulations can be admixed with other suitable pharmaceutically acceptable excipients used in parenteral dosage forms such as, but not limited to, preservatives.

The compounds and conjugates described herein can be used to treat various conditions that would benefit from an increased immune response, including but not limited to, cancers. Certain compounds and conjugates of the present disclosure can be superior in terms of efficacy expression, pharmacokinetics (e.g., absorption, distribution, metabolism, excretion), solubility (e.g., water solubility), interaction with other medicaments (e.g., drug-metabolizing enzyme inhibitory action), safety (e.g., acute toxicity, chronic toxicity, genetic toxicity, reproductive toxicity, cardiotoxicity, carcinogenicity, central toxicity) and/or stability (e.g., chemical stability, stability to an enzyme), and can be useful as a medicament. Accordingly, provided herein are compounds and conjugates of the present disclosure, as well as compositions comprising the same, for use in the preparation of a medicament, e.g., a medicament for the treatment of a condition that would benefit from an increased immune response, including but not limited to, cancer. Also provided herein are compounds and conjugates of the present disclosure, as well as compositions comprising the same, for use in treating a condition that would benefit from an increased immune response, including but not limited to, cancer.

The compounds and conjugates of the present disclosure can be used as medicaments such as an agents for the prophylaxis or treatment of diseases, for example, cancers. In some aspects, the cancer is a solid tumor cancer. In some aspects, the cancer is a hematological malignancy. In some aspects, the cancer is selected from the group consisting of an unresectable or metastatic, microsatellite instability-high (MSI-H) solid tumor, a mismatch repair deficient (dMMR) solid tumor, melanoma, non-small cell lung cancer (NSCLC), malignant pleural mesothelioma, renal cell carcinoma (RCC), classical Hodgkin lymphoma (cHL), squamous cell carcinoma of the head and neck (SCCHN), urothelial carcinoma, colorectal cancer, hepatocellular carcinoma (HCC), esophageal cancer, gastric cancer, gastroesophageal junction cancer, esophageal cancer, esophageal adenocarcinoma, and head and neck squamous cell cancer (HNSCC), primary mediastinal large b-cell lymphoma (PMBCL), microsatellite instability-high or mismatch repair deficient cancer, microsatellite instability-high or mismatch repair deficient colorectal cancer (CRC), gastric cancer, cervical cancer, merkel cell carcinoma (MCC), endometrial carcinoma, tumor mutational burden-high (TMB-H) cancer, cutaneous squamous cell carcinoma (cSCC), triple-negative breast cancer (TNBC), and basal cell carcinoma (BCC).

In some aspects, the cancer is resistant to an anti-PD1 therapy. The anti-PD1 therapy can be, for example, nivolumab, pembrolizumab, and/or cemiplimab.

In some aspects, the cancer is refractory to an anti-PD1 therapy. The anti-PD1 therapy can be, for example, nivolumab, pembrolizumab, and/or cemiplimab

In some aspects, the cancer is sensitive to an anti-PD1 therapy. The anti-PD1 therapy can be, for example, nivolumab, pembrolizumab, and/or cemiplimab.

In some aspects, the cancer is resistant to an anti-PDL1 therapy. The anti-PDL1 therapy can be, for example, durvalumab, atezolizumab, and/or avelumab.

In some aspects, the cancer is refractory to an anti-PDL1 therapy. The anti-PDL1 therapy can be, for example, durvalumab, atezolizumab, and/or avelumab.

In some aspects, the cancer is sensitive to an anti-PDL1 therapy. The anti-PDL1 therapy can be, for example, durvalumab, atezolizumab, and/or avelumab.

In some aspects, a compound or conjugate of the disclosure can be used in combination with a standard of care therapy, e.g., one or more therapeutic agents (e.g., anti-cancer agents and/or immunomodulating agents). Accordingly, in certain aspects, a method of treating a tumor disclosed herein comprises administering the compounds or conjugates of the disclosure in combination with one or more additional therapeutic agents. In some aspects, the compounds or conjugates of the disclosure can be used in combination with one or more anti-cancer agents, such that multiple elements of the immune pathway can be targeted. In some aspects, an anti-cancer agent comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody or antigen-binding fragment thereof), PD1 antagonist (e.g., anti-PD1 antibody or antigen-binding fragment thereof, anti-PD-L1 antibody or antigen-binding fragment thereof), TIM-3 antagonist (e.g., anti-TIM-3 antibody or antigen-binding fragment thereof), or combinations thereof. Additional example of immune checkpoint inhibitors include T-cell immunoglobulin and ITIM domain (TIGIT) antagonists, V-domain Ig suppressor of T-cell activation (VISTA) antagonists, B and T cell lymphocyte attenuator (BTLA) antagonists, and lymphocyte activation gene-3 (LAG-3) antagonists.

In some aspects, the compound or conjugate of the disclosure is administered to the subject prior to or after the administration of the additional therapeutic agent. In other aspects, the compound or conjugate of the disclosure is administered to the subject concurrently with the additional therapeutic agent. In certain aspects, the compound or conjugate of the disclosure and the additional therapeutic agent can be administered concurrently as a single composition in a pharmaceutically acceptable carrier. In other aspects, the compound or conjugate of the disclosure and the additional therapeutic agent are administered concurrently as separate compositions.

The compounds and conjugates of the present disclosure can be used as medicaments such as an agent for the prophylaxis or treatment of additional conditions that would benefit from an increased immune response. Conditions that would benefit from an increased immune response include, for example, infections (e.g., viral, bacterial, and/or parasitic infections), immunosuppressive diseases or disorders, and multiple sclerosis.

In some aspects, a subject that can be treated with the compound conjugate of the present disclosure is a nonhuman animal such as a rat or a mouse. In some aspects, the subject that can be treated is a human.

In some aspects, the compounds or conjugates of the present disclosure can be used to increase immune cell activity (e.g., natural killer (NK) cell activity or T cell activity, including effector T cell activity), e.g., by contacting the immune cell with a compound or conjugate of the present disclosure. The contacting can be in vitro or in vivo. In aspects where the contacting is in vivo, the contacting can be in a subject, e.g., a human subject that has cancer or another condition that would benefit from an increased immune response. The increased immune cell activity can treat the cancer or other condition that would benefit from an increased immune response.

In some aspects, the compounds or conjugates of the present disclosure can be used to increase immune cell (e.g., NK cell or T cell, including effector T cell) proliferation e.g., by contacting the immune cell with a compound or conjugate of the present disclosure. The contacting can be in vitro or in vivo. In aspects where the contacting is in vivo, the contacting can be in a subject, e.g., a human subject that has cancer or another condition that would benefit from an increased immune response. The increased immune cell proliferation can treat the cancer or other condition that would benefit from an increased immune response.

In some aspects, the compounds or conjugates of the present disclosure can be used to increase migration of an immune cell (e.g., NK cell or T cell, including effector T cell) to a tumor cell, e.g. by contacting the immune cell with a compound or conjugate of the present disclosure. The contacting can be in vitro or in vivo. In aspects where the contacting is in vivo, the contacting can be in a subject, e.g., a human subject that has cancer. The increased immune cell migration can treat the cancer.

In some aspects, the compounds or conjugates of the present disclosure can be used to reduce exhaustion of an immune cell (e.g., NK cell or T cell, including effector T cell), e.g., by contacting the immune cell with a compound or conjugate of the present disclosure. The contacting can be in vitro or in vivo. In aspects where the contacting is in vivo, the contacting can be in a subject, e.g., a human subject that has cancer or another condition that would benefit from an increased immune response. The reduced immune cell exhaustion can treat the cancer or other condition that would benefit from an increased immune response.

In some aspects, the compounds or conjugates of the present disclosure can be used to increase secretion of IFN-y or IL-2 from an immune cell (e.g., NK cell or T cell, including effector T cell), e.g,. by contacting the immune cell with a compound or conjugate of the present disclosure. The contacting can be in vitro or in vivo. In aspects where the contacting is in vivo, the contacting can be in a subject, e.g., a human subject that has cancer or another condition that would benefit from an increased immune response. The increased secretion of IFN-y or TL-2 can treat the cancer or other condition that would benefit from an increased immune response.

In some of the above aspects, the immune cell is an effector T cell. According, in some aspects, the compounds or conjugates of the present disclosure can be used to increase effector T cell activity, e.g., by contacting an effector T cell with a compound or conjugate of the present disclosure. The contacting can be in vitro or in vivo. In aspects where the contacting is in vivo, the contacting can be in a subject, e.g., a human subject, that has cancer. The increased effector T cell activity can treat the cancer.

In some aspects, the compounds or conjugates of the present disclosure can be used to increase effector T cell proliferation, e.g., by contacting an effector T cell with a compound or conjugate of the present disclosure. The contacting can be in vitro or in vivo. In aspects where the contacting is in vivo, the contacting can be in a subject, e.g., a human subject, that has cancer. The increased effector T cell proliferation can treat the cancer.

In some aspects, the compounds or conjugates of the present disclosure can be used to increase migration of an effector T cell to a tumor cell, e.g. by contacting an effector T cell with a compound or conjugate of the present disclosure. The contacting can be in vitro or in vivo. In aspects where the contacting is in vivo, the contacting can be in a subject, e.g., a human subject, that has cancer. The increased effector T cell migration can treat the cancer.

In some aspects, the compounds or conjugates of the present disclosure can be used to reduce effector T cell exhaustion, e.g., by contacting an effector T cell with a compound or conjugate of the present disclosure. The contacting can be in vitro or in vivo. In aspects where the contacting is in vivo, the contacting can be in a subject, e.g., a human subject, that has cancer. The reduced effector T cell exhaustion can treat the cancer.

In some aspects, the conjugates of the present disclosure can be used to deliver a payload that is capable of activating an effector T cell to an effector T cell, e.g., by contacting an effector T cell with a conjugate of the present disclosure. The contacting can be in vitro or in vivo. In aspects where the contacting is in vivo, the contacting can be in a subject, e.g., a human subject, that has cancer. The delivery of the payload to effector T cells can treat the cancer.

### V. Methods of Preparing Conjugates

The present disclosure further provides methods of preparing the conjugates, the process comprising reacting a binding moiety with a payload as described herein or payload-linker precursor. As used herein, the term “linker precursor” refers to a compound which, when reacted with a binding moiety as described herein, connects the binding moiety to the payload.

In certain aspects, the linker precursor can be selected from:

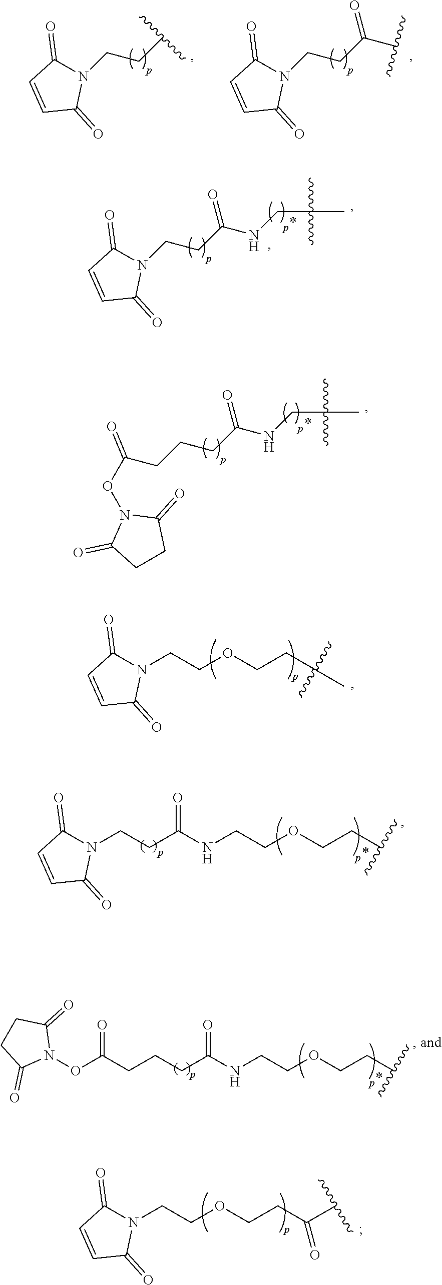

wherein
p is an integer from 1 to 10;
p* is an integer from 1 to 10; and
[symbol] is the point of attachment to the payload.

In certain aspects, the linker precursor can be selected from:

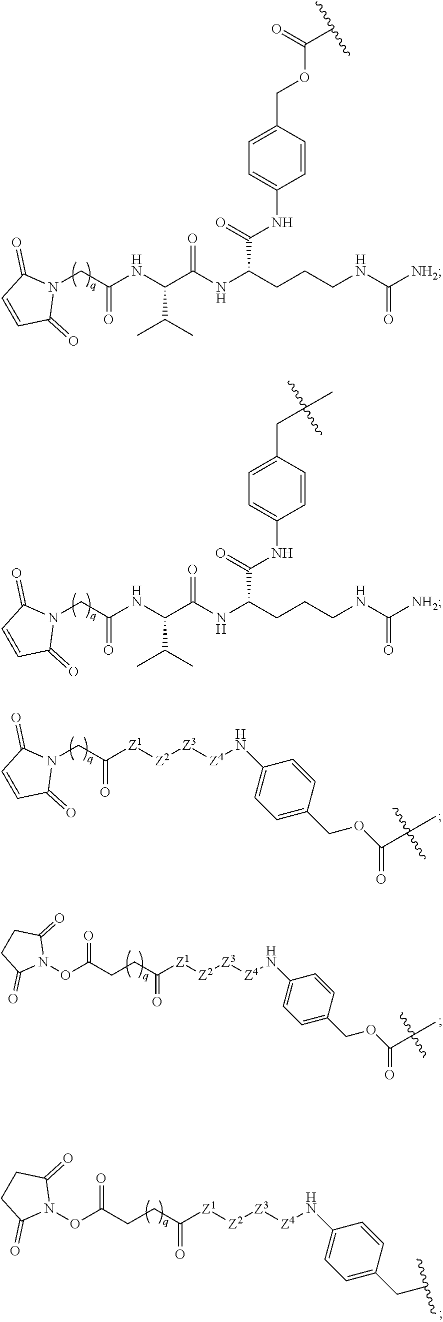

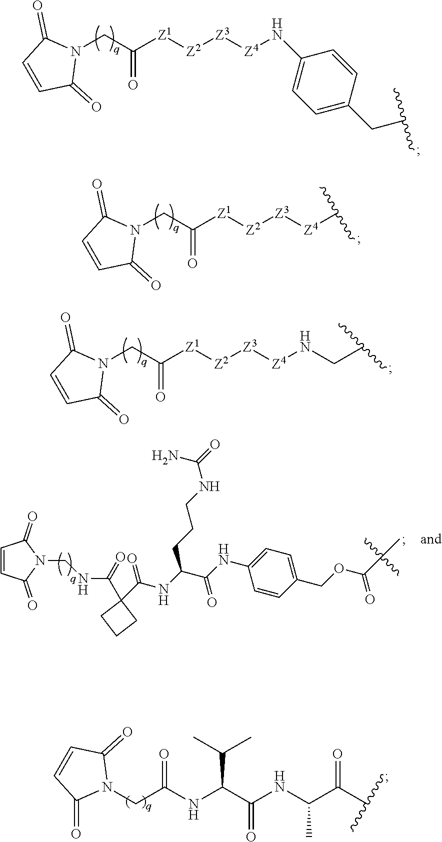

wherein:
   q is an integer from 2 to 10;
   Z¹, Z², Z³, and Z⁴ are each independently absent or a naturally-occurring amino acid residue in the L- or D-configuration, provided that at least two of Z¹, Z², Z³, and Z⁴ are amino acid residues; and
   [symbol] is the point of attachment to the payload.

In certain aspects, Z¹, Z², Z³, and Z⁴ are independently absent or selected from the group consisting of L-valine, D-valine, L-citrulline, D-citrulline, L-alanine, D-alanine, L-glutamine, D-glutaimine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, and Z⁴ are amino acid residues.

In some aspects, Z¹ is absent or glycine; Z² is absent or selected from the group consisting of L-glutamine, D-glutamine, L-glutamic acid, D-glutamic acid, L-aspartic acid, D-aspartic acid, L-alanine, D-alanine, and glycine; Z³ is selected from the group consisting of L-valine, D-valine, L-alanine, D-alanine, L-phenylalanine, D-phenylalanine, and glycine; and Z⁴ is selected from the group consisting of L-alanine, D-alanine, L-citrulline, D-citrulline, L-asparagine, D-asparagine, L-lysine, D-lysine, L-phenylalamine, D-phenylalanine, and glycine.

In some aspects, the linker precursor can be selected from:

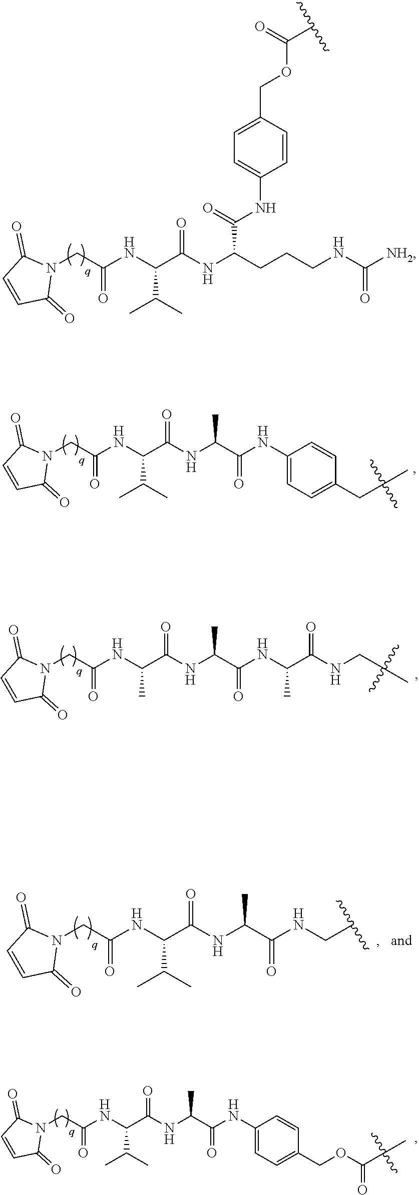

wherein:
   q is an integer from 2 to 10;
   [symbol] is the point of attachment to the payload.

In certain aspects, the linker precursor can be selected from

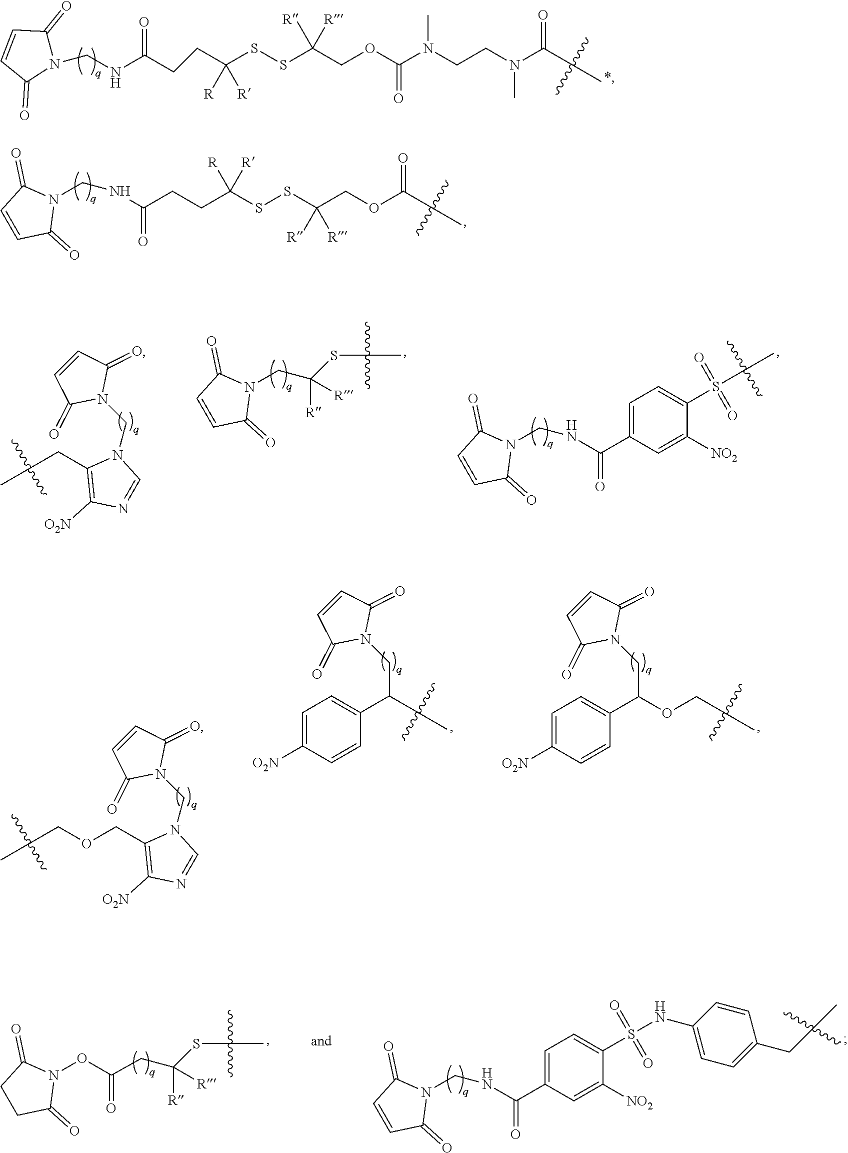

wherein:
   q is an integer from 2 to 10;
   R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring; and
   [symbol] is the point of attachment to the payload.

In certain aspects, the linker precursor cam be selected from

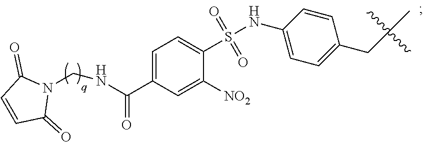

wherein:
   q is an integer from 2 to 10; and
   [symbol] is the point of attachment to the payload.

In certain aspects, the linker precursor can be selected from

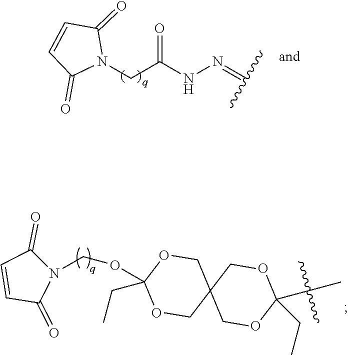

wherein:
   q is an integer from 2 to 10;
   [symbol] is the point of attachment to the payload.

In some aspects, the linker precursor can be selected from

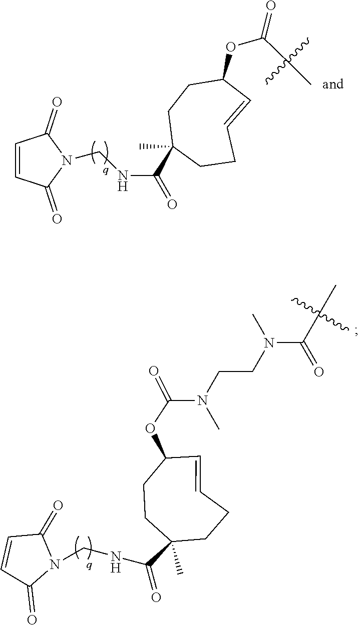

wherein:
   q is an integer from 2 to 10; and
   [symbol] is the point of attachment to the payload.

In some aspects, the linker precursor can be

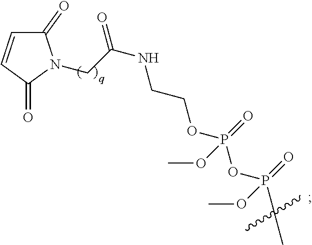

wherein:
   q is an integer from 2 to 10;
   [symbol] is the point of attachment to the payload.

In certain aspects, the linker precursor can be selected from

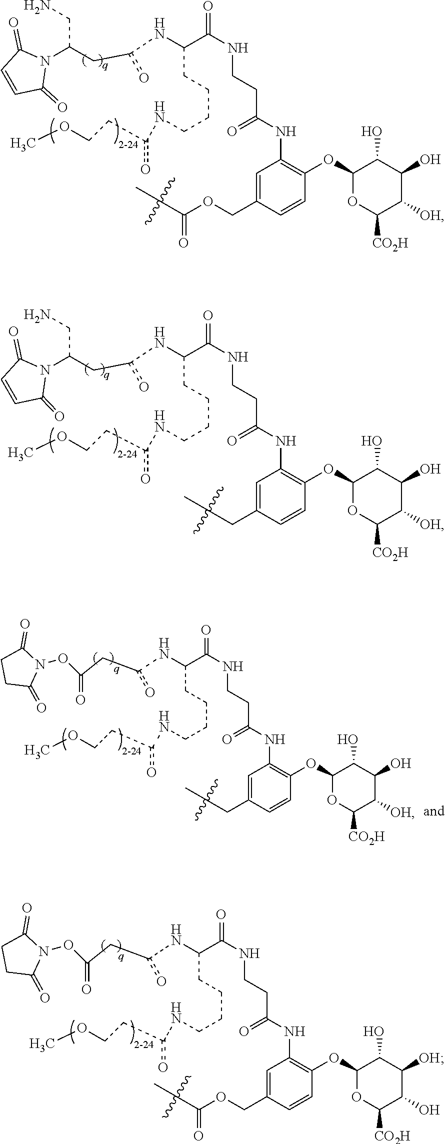

wherein:
   q is an integer from 2 to 10;
   [symbol] is absent or a bond;
   [symbol] is the point of attachment to the payload.

In some aspects, the linker precursor can be

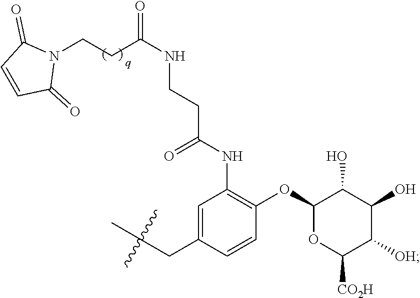

wherein:
   q is an integer from 2 to 10;
   [symbol] is absent or a bond; and
   [symbol] is the point of attachment to the payload.

In some aspects, the binding moiety is pre-treated before it is reacted with the payload or the payload-linker precursor. In certain aspects, the payload or payload-linker precursor is reacted with a binding moiety, which comprises an antibody or an antigen binding portion thereof. In aspects where the binding moiety is an antibody, the antibody can be pretreated to reduce interchain disulfides prior to reaction with the payload or payload-linker precursor.

The embodiments described herein are further detailed with reference to the examples shown below. These examples are provided for the purpose of illustration only and the embodiments described herein should in no way be construed as being limited to these examples. Rather, the embodiments should be construed to encompass any and all variations which become evident as a result of the teachings provided herein.

### Examples

### General Synthetic Methods and Intermediates

The compounds of the present disclosure can be prepared by one of ordinary skill in the art in light of the present disclosure and knowledge in the art, and/or by reference to the schemes shown below and the synthetic examples. Exemplary synthetic routes are set forth in Schemes below and in Examples. It should be understood that the variables, (for example “R” groups) appearing in the following schemes and examples are to be read independently from those appearing elsewhere in the application. One of ordinary skill in the art would readily understand how the schemes and examples shown below illustrate the preparation of the compounds described herein.

Abbreviations used in the schemes generally follow conventions used in the art. Chemical abbreviations used in the specification and examples are defined as follows:

“DMF” for N,N-dimethylformamide; “DIBAL-H” or “DIBAl-H” for diisobutylaluminum hydride; “DCM” for dichlormethane; “Ph” for phenyl; “DEAD” for diethyl azodicarboxylate; “NMO” for N-methylmorpholine N-oxide; “THF” for tetrahydrofuran; “t-Bu” for tert-butyl; “HATU” for 1-[bis(dimethylamino)methylene]-1H-1,2,3-triazolo[4,5-b]pyridinium 3-oxid hexafluorophosphate or N-[(dimethylamino)-1H-1,2,3-triazolo-[4,5-b]pyridin-1-ylmethylene]-N-methylmethanaminium hexafluorophosphate N-oxide; “DIEA,” “DIPEA,” and “iPrNEt₂” for diisopropylethylamine; “PE” for petroleum ether; “EA” or “EtOAc” for ethyl acetate; “Ac” for acetyl; “BPO” for benzoyl peroxide; “AIBN” for azobisisobutyronitrile; “o/n” for overnight; “r.t.” or “rt” or “RT” for room temperature or retention time (context will dictate); “IBX” for 2-iodoxybenzoic acid; “STAB” for sodium triacetoxyborohydride; “Me” for methyl “NBS” for N-bromosuccinimide; “dtbbpy” for 4,4′-di-tert-butyl-2,2′-bipyridyl; “TFA” for trifluoroacetic acid; “DMA” for N,N-dimethylacetamide; “Et₃N” and “TEA” for trimethylamine; “Ts” for p-toluenesulfonyl; “Trt” for triphenylmethyl; “TIPS” for triisopropyl silyl; “Et” for ethyl; “LiHMDS” for lithium hexamethyldisilazide; “MeCN” or “ACN” for acetonitrile; “TMS” for trimethylsilyl; “NIS” for N-iodosuccinimide; “BOC” or “Boc” for tert-butoxycarbonyl; “DMSO” for dimethylsulfoxide; and “HOBt” or “HOBT” for 1-hydroxybenzotriazole hydrate, “h” for hours; and “min” for minutes.

### Example 1: Preparation of Cbl-b Inhibitors

### Synthesis of INT3

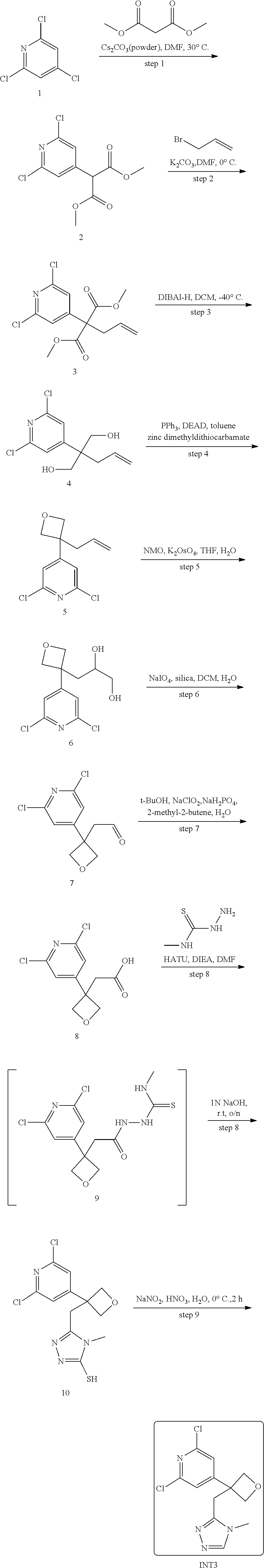

### Step 1: Synthesis of Compound 2

To a stirred mixture of 2,4,6-trichloropyridine (Compound 1, 100 g, 548.15 mmol, 1 equiv) and dimethyl malonate (86.90 g, 657.78 mmol, 1.2 equiv) in DMF (1000 mL) was added Cs₂CO₃ (535.80 g, 1644.46 mmol, 3 equiv, powder) in portions at room temperature under air atmosphere. The resulting mixture was stirred for 2 days at room temperature under air atmosphere. LCMS indicated the reaction was completed. The resulting mixture was filtered, the filter cake was washed with EtOAc (3×500 mL). The combined organic layers were washed with water (5×500 mL), dried over anhydrous Na₂SO₄. After filtration, the filtrate was concentrated under reduced pressure. The resulting mixture was concentrated under vacuum to afford 1,3-dimethyl 2-(2,6-dichloropyridin-4-yl)propanedioate (Compound 2, 95 g, 62%) as a yellow oil. LCMS: (ES, m/s): 278,280 [M+H]⁺.

### Step 2: Synthesis of Compound 3

To a stirred mixture of 1,3-dimethyl 2-(2,6-dichloropyridin-4-yl)propanedioate (Compound 2, 95 g, 341.61 mmol, 1 equiv) and K₂CO₃ (141.64 g, 1024.84 mmol, 3 equiv) in DMF (950 mL) was added allyl bromide (82.66 g, 683.23 mmol, 2 equiv) dropwise 0° C. under air atmosphere. The resulting mixture was stirred for overnight at 0° C. under air atmosphere. LCMS indicated the reaction was completed. The resulting mixture was filtered, the filter cake was washed with EtOAc (3×400 mL). The combined organic layers were washed with water (4×400 mL), dried over anhydrous Na₂SO₄. After filtration, the filtrate was concentrated under reduced pressure. This resulted in 1,3-dimethyl 2-(2,6-dichloropyridin-4-yl)-2-(prop-2-en-1-yl)propanedioate (Compound 3, 95 g, 76%) as a yellow oil. LCMS:(ES·m/z):318,320[M+1]⁺.

### Step 3. Synthesis of Compound 4

To a stirred solution of 1,3-dimethyl 2-(2,6-dichloropyridin-4-yl)-2-(prop-2-en-1-yl)propanedioate (Compound 3, 70 g, 220.02 mmol, 1.00 equiv) in DCM (700 mL) was added DIBAl-H (880.09 mL, 1N in DCM, 880.09 mmol, 4 equiv) dropwise at −40° C. under nitrogen atmosphere. The resulting mixture was stirred for 1 h at −40° C. under nitrogen atmosphere. LCMS indicated complete reaction. The reaction was quenched by the addition of MeOH (20 mL) at room temperature. The resulting mixture was filtered, the filter cake was washed with EtOAc (3×50 mL). The filtrate was concentrated under reduced pressure to afford 2-(2,6-dichloropyridin-4-yl)-2-(prop-2-en-1-yl)propane-1,3-diol (Compound 4, 50 g, 69%) as a yellow oil. LCMS (ESI, ms):262,264[M+H]⁺

### Step 4. Synthesis of Compound 5

To a stirred mixture of 2-(2,6-dichloropyridin-4-yl)-2-(prop-2-en-1-yl)propane-1,3-diol (Compound 4, 70 g, 267.04 mmol, 1 equiv) and PPh₃ (140.09 g, 534.08 mmol, 2 equiv) in toluene (700 mL) in portions at 0° C. under N₂ atmosphere. The resulting mixture was stirred for 10 min at 0° C. under N₂ atmosphere. To the above mixture was added ziram (122.49 g, 400.56 mmol, 1.5 equiv) and DEAD (93.01 g, 534.08 mmol, 2 equiv) dropwise over 30 min at 0° C. The resulting mixture was stirred for additional overnight at room temperature. LCMS indicated the reaction was completed. The resulting mixture was filtered, the filter cake was washed with EtOAc (3×100 mL). The filtrate was concentrated under reduced pressure. The residue was purified by silica gel column chromatography, eluted with PE/EA (5:1) to afford 2,6-dichloro-4-[3-(prop-2-en-1-yl)oxetan-3-yl]pyridine (Compound 5, 37 g, 50%) as a yellow oil. LCMS:(ES·m/z):244,246[M+1]⁺.

### Step 5: Synthesis of Compound 6

To a stirred solution of 2,6-dichloro-4-[3-(prop-2-en-1-yl)oxetan-3-yl]pyridine (Compound 5, 37 g, 151.56 mmol, 1 equiv) in THE (240 mL) and H₂O (120 mL) was added NMO (53.27 g, 454.69 mmol, 3 equiv) in portions and was added K20SO₄ (2.8 g, 9.00 mmol, 0.05 equiv) in portions at 0° C. under air atmosphere. The resulting mixture was stirred for overnight at room temperature under nitrogen atmosphere. LCMS indicated the reaction was completed. The reaction was quenched with Na₂S₂O₃ at 0° C. The resulting mixture was filtered, the filter cake was washed with DCM (3×100 mL). The filtrate was concentrated under reduced pressure. The crude product used in the next step directly without further purification. This resulted in 3-[3-(2,6-dichloropyridin-4-yl)oxetan-3-yl]propane-1,2-diol (Compound 6, 38 g, 90%) as a black solid. LCMS:(ES·m/z):278,280[M+1]⁺.

### Step 6: Synthesis of Compound 7

The mixture of NaIO₄ (58.45 g, 273.25 mmol, 2.00 equiv) and silica (76 g) in H₂O (190 mL) was stirred for 30 min at room temperature under air atmosphere. Then a stirred solution of 3-[3-(2,6-dichloropyridin-4-yl)oxetan-3-yl]propane-1,2-diol (Compound 6, 38 g, 136.62 mmol, 1 equiv) in DCM (380 mL) was added dropwise at room temperature. The resulting mixture was stirred for 2 h at room temperature. LCMS indicated the reaction was completed. The resulting mixture was filtered, the filter cake was washed with DCM (3×150 mL). The filtrate was concentrated under reduced pressure. The crude product was used in the next step directly without further purification. This resulted in 2-[3-(2,6-dichloropyridin-4-yl)oxetan-3-yl]acetaldehyde (Compound 7, 27 g, 80%) as a brown solid. LCMS:(ES·m/z):246,248[M+1]⁺.

### Step 7: Synthesis of Compound 8

To a stirred solution of 2-[3-(2,6-dichloropyridin-4-yl)oxetan-3-yl]acetaldehyde (Compound 7, 27 g, 109.71 mmol, 1 equiv) in t-BuOH (270 mL) and H₂O (135 mL) was added NaClO₂ (34.73 g, 384.00 mmol, 3.5 equiv) and NaH₂PO₄ (26.33 g, 219.43 mmol, 2 equiv) in portions at 0° C. under air atmosphere. To a stirred mixture was added 2-methyl-2-butene (142 mL) dropwise at 0° C. under nitrogen atmosphere. The resulting mixture was stirred for overnight at room temperature under nitrogen atmosphere. LCMS indicated the reaction was completed. The mixture was acidified to pH 3 with 1N HCl. The precipitated solids were collected by filtration and washed with Et₂O (3×100 mL). The filtrate was concentrated under reduced pressure. The residue was purified by trituration with Et₂O (50 mL). This resulted in [3-(2,6-dichloropyridin-4-yl)oxetan-3-yl]acetic acid (Compound 8, 14.4 g, 50%) as a white solid. LCMS:(ES·m/z):262,264[M+1]⁺.

### Step 9: Synthesis of Compound 10

To a stirred mixture of [3-(2,6-dichloropyridin-4-yl)oxetan-3-yl]acetic acid (Compound 8, 14 g, 53.41 mmol, 1 equiv) and 1-amino-3-methylthiourea (6.18 g, 58.75 mmol, 1.1 equiv) in DMF (120 mL) was added HATU (24.37 g, 64.10 mmol, 1.2 equiv) in portions and DIEA (20.71 g, 160.25 mmol, 3 equiv) dropwise at 0° C. under air atmosphere. The resulting mixture was stirred for overnight at room temperature. To the above mixture was added NaOH (1N, 100 mL) dropwise over 10 min at 0° C. The resulting mixture was stirred for additional 1 h at 0° C. LCMS indicated the reaction was completed. The mixture was acidified to pH 3 with 1N HCl. The aqueous layer was extracted with EtOAc (3×150 mL). The combined organic was concentrated under reduced pressure. The residue was purified by trituration with Et₂O (80 mL). This resulted in 5-{[3-(2,6-dichloropyridin-4-yl)oxetan-3-yl]methyl}-4-methyl-1,2,4-triazole-3-thiol (Compound 10, 14 g, 77%) as a white solid. LCMS:(ES·m/z): 331,333[M+1]⁺.

### Step 9: Synthesis of INT3

To a stirred mixture of 5-{[3-(2,6-dichloropyridin-4-yl)oxetan-3-yl]methyl}-4-methyl-1,2,4-triazole-3-thiol (Compound 10, 12 g, 36.23 mmol, 1 equiv) and NaNO₂ (7.50 g, 108.69 mmol, 3 equiv) in THF (120 mL) was added HNO₃ (1N, 119.92 mL, 119.92 mmol, 3.31 equiv) dropwise at −20° C. under air atmosphere. The resulting mixture was stirred for 2 h at 0° C. LCMS indicated the reaction was completed. The reaction mixture was concentrated under reduced pressure. The mixture was basified to pH 8 with saturated Na₂CO₃ (aq.). The resulting mixture was extracted with CH₂Cl₂ (3×100 mL). The combined organic layers were washed with CH₂Cl₂ (2×100 mL), dried over anhydrous Na₂SO₄. After filtration, the filtrate was concentrated under reduced pressure. The residue was purified by silica gel column chromatography, eluted with CH₂Cl₂/MeOH (10:1) to afford 2,6-dichloro-4-{3-[(4-methyl-1,2,4-triazol-3-yl)methyl]oxetan-3-yl}pyridine (INT3, 5.6 g, 47%) as a white solid. LCMS:(ES·m/z):299,301[M+1]⁺; ¹H-NMR (300 MHz, DMSO-d₆) 8.37 (s, 1H), 7.58 (s, 2H), 4.98-4.70 (m, 4H), 3.65 (s, 2H), 3.54 (s, 2H).

### Synthesis of INT4

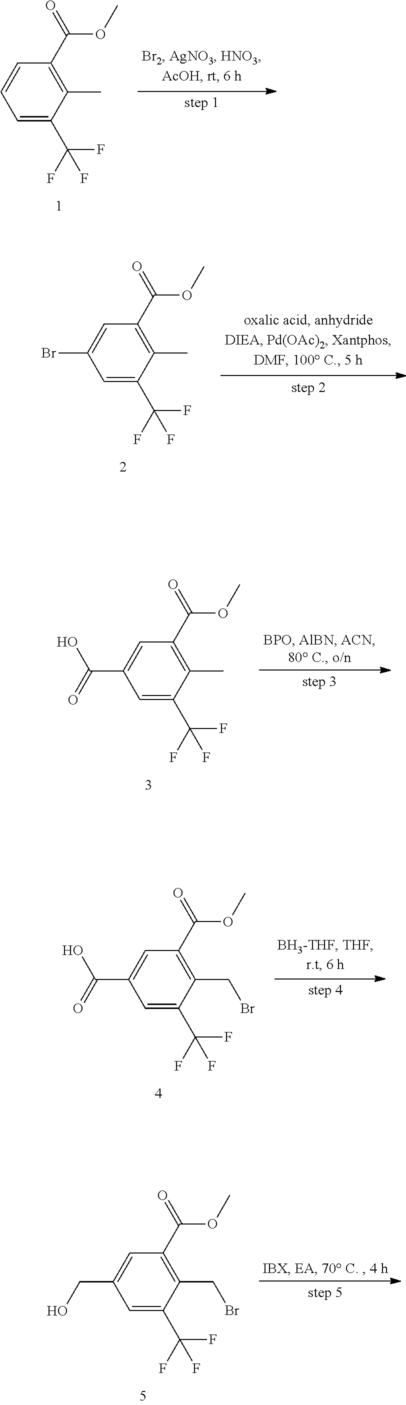 [SMILES: COC(=O)c1cccc(C(F)(F)F)c1C ; COC(=O)c1cc(Br)cc(C(F)(F)F)c1C ; COC(=O)c1cc(C(=O)O)cc(C(F)(F)F)c1C ; COC(=O)c1cc(C(=O)O)cc(C(F)(F)F)c1CBr ; COC(=O)c1cc(CO)cc(C(F)(F)F)c1CBr]

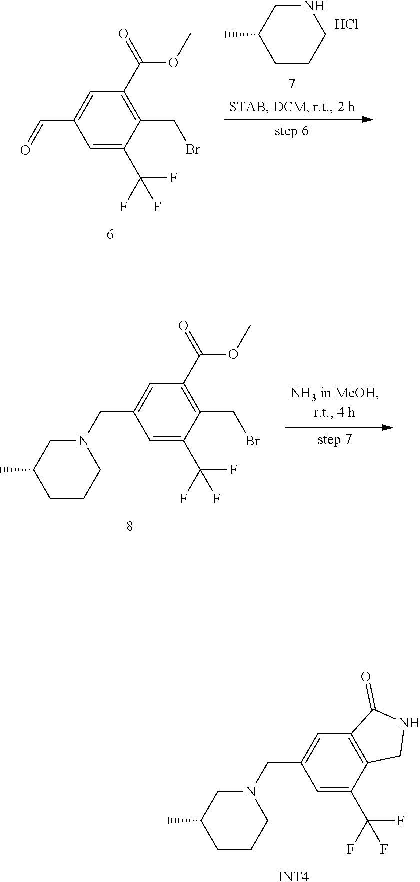

### Step 1: Synthesis of Compound 2

To a stirred mixture of methyl 2-methyl-3-(trifluoromethyl)benzoate (Compound 1, 44 g, 201.67 mmol, 1 equiv) in AcOH (286 mL) was added HNO₃ (127 g, 2015.46 mmol, 9.99 equiv) dropwise over 20 min at 10° C. under nitrogen atmosphere. To the above mixture was added Br₂ (35.5 g, 222.14 mmol, 1.10 equiv) dropwise over 10 min at 10° C. The resulting mixture was stirred for additional 10 min at 10° C. To the above mixture was added AgNO₃ (44.4 g, 261.37 mmol, 1.30 equiv) in water (105 mL) (2.5 mol/L) dropwise over 25 min at 10° C. The resulting mixture was stirred for additional overnight at room temperature. TLC indicated the reaction was completed. The mixture was basified to pH 8 with saturated Na₂CO₃ (aq.). The resulting mixture was extracted with EtOAc (3×800 mL). The combined organic layers were washed with water (3×100 mL), dried over anhydrous Na₂SO₄. After filtration, the filtrate was concentrated under reduced pressure. The residue was purified by silica gel column chromatography, eluted with PE/EA (10:1) to afford methyl 5-bromo-2-methyl-3-(trifluoromethyl)benzoate (Compound 2, 42 g, 70%) as a yellow oil. ¹H NMR (300 MHz, DMSO-d₆) δ 8.32 (s, 1H), 8.08 (s, 1H), 3.87 (s, 3H), 3.46-3.44 (m, 3H).

### Step 2: Synthesis of Compound 3

To a stirred mixture of methyl 5-bromo-2-methyl-3-(trifluoromethyl)benzoate (Compound 2, 42 g, 141.38 mmol, 1.00 equiv) and acetic anhydride (21.7 g, 212.56 mmol, 1.50 equiv) in DMF (420 mL, 5427.13 mmol, 38.39 equiv) was added oxalic acid (19.8 g, 219.91 mmol, 1.56 equiv) and Pd(OAc)₂ (3.2 g, 14.25 mmol, 0.10 equiv) and Xantphos (16.5 g, 28.51 mmol, 0.20 equiv) in portions at room temperature. The mixture was added DIEA (42 mL, 241.12 mmol, 1.71 equiv) dropwise at room temperature. The resulting mixture was stirred for 4 h at 100° C. under nitrogen atmosphere. The mixture was allowed to cool down to room temperature. LCMS indicated the reaction was completed.

The resulting mixture was quenched with water (1 L) and extracted with EtOAc (3×800 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (1:1) to afford 3-(methoxycarbonyl)-4-methyl-5-(trifluoromethyl)benzoic acid (Compound 3, 29 g, 78.24%) as an off-white solid. LCMS:(ES·m/z):261[M−1]⁻.

### Step 3: Synthesis of Compound 4

To a stirred mixture of 3-(methoxycarbonyl)-4-methyl-5-(trifluoromethyl)benzoic acid (29 g, 110.60 mmol, 1 equiv) in ACN (580 mL) was added NBS (29.53 g, 165.91 mmol, 1.50 equiv) and BPO (8.03 g, 33.18 mmol, 0.3 equiv) in portions at room temperature. The resulting mixture was stirred for overnight at 80° C. under nitrogen atmosphere. The mixture was allowed to cool down to room temperature. LCMS indicated the reaction was completed. The resulting mixture was concentrated under reduced pressure. The residue was purified by silica gel column chromatography, eluted with PE/EA (1:1) to afford 4-(bromomethyl)-3-(methoxycarbonyl)-5-(trifluoromethyl)benzoic acid (Compound 4, 32 g, 84%) as a yellow solid. LCMS:(ES·m/z):339[M−1]⁻. ¹H NMR (300 MHz, DMSO-d₆) δ 8.57 (s, 1H), 8.35 (s, 1H), 5.07 (s, 2H), 3.99 (s, 3H).

### Step 4: Synthesis of Compound 5

To a stirred mixture of 3-(methoxycarbonyl)-4-methyl-5-(trifluoromethyl)benzoic acid (Compound 4, 32 g, 122.05 mmol, 1.00 equiv) in THE (320 mL) was added BH₃-THF (235 mL, 1 mol/L, 235 mmol, 2.0 equiv) dropwise at 0° C. The resulting mixture was stirred for 6 h at room temperature. LCMS indicated the reaction was completed. The reaction was quenched with MeOH and stirred for 1 h at room temperature. The resulting mixture was concentrated under reduced pressure. The residue was purified by silica gel column chromatography, eluted with PE/EA (1:1) to afford methyl 2-(bromomethyl)-5-(hydroxymethyl)-3-(trifluoromethyl)benzoate (Compound 5, 23.49 g, 58%) as a yellow oil. LCMS:(ES·m/z):245[M−1−Br]⁻. ¹H NMR (300 MHz, DMSO-d₆) δ 8.05 (s, 1H), 7.91 (s, 1H), 5.04 (s, 2H), 4.63 (s, 2H), 3.92 (s, 3H).

### Step 5: Synthesis of Compound 6

To a stirred solution of methyl 2-(bromomethyl)-5-(hydroxymethyl)-3-(trifluoromethyl)benzoate (Compound 5, 23.49 g, 71.81 mmol, 1.00 equiv) in EA (240 mL) was added IBX (30.16 g, 107.72 mmol, 1.5 equiv) in portions at room temperature. The resulting mixture was stirred for 3 h at 70° C. The mixture was allowed to cool down to room temperature. LCMS indicated the reaction was completed. The resulting mixture was filtered, the filter cake was washed with EtOAc (3×50 mL). The filtrate was washed with Sat·Na₂S204 (3×30 mL), dried over anhydrous Na₂SO₄. After filtration, the filtrate was concentrated under reduced pressure. The residue was purified by silica gel column chromatography, eluted with PE/EA (1:1) to afford methyl 2-(bromomethyl)-5-formyl-3-(trifluoromethyl)benzoate (Compound 6, 20 g, 85%) as a yellow oil. LCMS:(ES·m/z):323[M−1]⁻. ¹H NMR (300 MHz, DMSO-d₆) δ 10.13 (s, 1H), 8.57 (s, 1H), 8.46 (s, 1H), 5.08 (s, 2H), 3.97 (s, 3H).

### Step 6: Synthesis of Compound 7

To a stirred mixture of methyl 2-(bromomethyl)-5-formyl-3-(trifluoromethyl)benzoate (Compound 6, 5.17 g, 15.90 mmol, 1.00 equiv) in DCM (31.7 mL) was added (3S)-3-methylpiperidine hydrochloride (2.37 g, 17.49 mmol, 1.1 equiv) and STAB (13.48 g, 63.61 mmol, 4 equiv) in portions at 0° C. The resulting mixture was stirred for 2 h at room temperature. The reaction was quenched by the addition of MeOH (5 mL) at 0° C. The resulting mixture was concentrated under reduced pressure and quenched with water (30 mL). The resulting mixture was extracted with DCM (3×30 mL). The combined organic layers were washed with water (3×10 mL), dried over anhydrous Na₂SO₄. After filtration, the filtrate was concentrated under reduced pressure. The residue was purified by reverse flash chromatography with the following conditions: C18 column, ACN in water (0.1% NH₄HCO₃), 10% to 80% gradient in 50 min; detector, UV 254 nm. The collected fraction was concentrated to afford methyl 2-(bromomethyl)-5-{[(3S)-3-methylpiperidin-1-yl]methyl}-3-(trifluoromethyl)benzoate (Compound 8, 3.0 g, 46%) as an off-white oil. LCMS:(ES·m/z):408, 410[M+1]⁺.

### Step 7. Synthesis of INT4

To a stirred mixture of methyl 2-(bromomethyl)-5-{[(3S)-3-methylpiperidin-1-yl]methyl}-3-(trifluoromethyl)benzoate (Compound 8, 1.7 g, 4.16 mmol, 1 equiv) in NH₃ (g) in MeOH (7 M, 17 mL, 119 mmol) at 0° C. The resulting mixture was stirred for 5 h at room temperature. LCMS indicated the reaction was completed. The resulting mixture was concentrated under reduced pressure. The residue was purified by reverse flash chromatography with the following conditions: C18 column; mobile phase, ACN in water (0.05% NH₄HCO₃), 10% to 100% gradient in 40 min; detector, UV 254 nm. The resulting mixture was concentrated under reduced pressure. This resulted in 6-{[(3S)-3-methylpiperidin-1-yl]methyl}-4-(trifluoromethyl)-2,3-dihydroisoindol-1-one (INT4, 507.6 mg, 38%) as a yellow solid. LCMS:(ES·m/z):313[M+1]⁺. ¹H NMR (300 MHz, DMSO-d₆) δ 8.87 (s, 1H), 7.87 (d, J=11.4 Hz, 2H), 4.53 (s, 2H), 3.61 (s, 2H), 2.72-2.67 (m, 2H), 1.99-1.87 (m, 1H), 1.77-1.40 (m, 5H), 0.90-0.60 (m, 4H).

### Synthesis of Compound 100

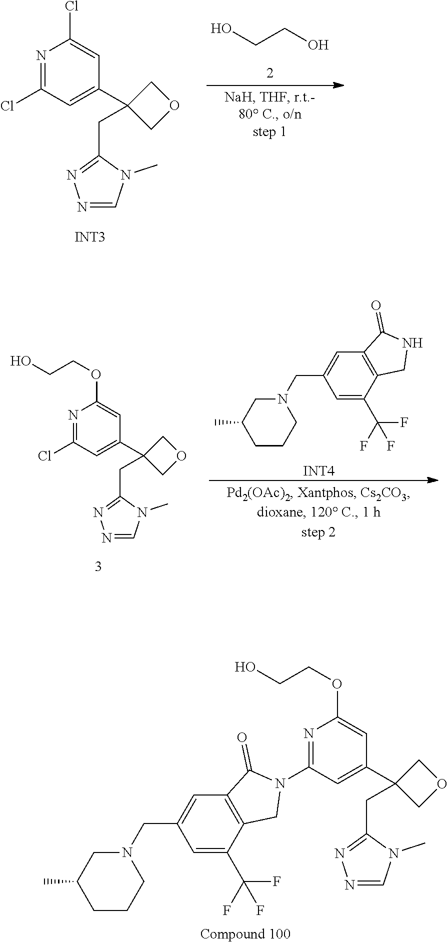

### Step 1: Synthesis of Compound 3

To a solution of ethylene glycol (Compound 2, 124 mg, 2.01 mmol, 3.00 equiv) in THE (10 mL) was added NaH (40 mg, 60%, 1.00 mmol, 1.50 equiv) at room temperature under N₂. The resulting mixture was stirred at room temperature for 0.5 h under N₂. Then 2,6-dichloro-4-{3-[(4-methyl-1,2,4-triazol-3-yl)methyl]oxetan-3-yl}pyridine (INT3, 200 mg, 0.67 mmol, 1.00 equiv) was added at room temperature under N₂. The resulting mixture was stirred at room temperature overnight. LCMS indicated the reaction was completed. The resulting mixture was diluted with water (20 mL). The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified with Prep-TLC (DCM: MeOH=10:1) to give 2-((6-chloro-4-(3-((4-methyl-4H-1,2,4-triazol-3-yl)methyl)oxetan-3-yl)pyridin-2-yl)oxy)ethan-1-ol (150 mg, 65%) as a white solid. LCMS (ES, m/z): 325,327 [M+H]⁺

### Step 2: Synthesis of Compound 100

A solution of 2-[(6-chloro-4-{3-[(4-methyl-1,2,4-triazol-3-yl)methyl]oxetan-3-yl}pyridin-2-yl)oxy]ethanol (Compound 3, 140 mg, 0.43 mmol, 1.00 equiv), 6-{[(3S)-3-methylpiperidin-1-yl]methyl}-4-(trifluoromethyl)-2,3-dihydroisoindol-1-one (INT4, 135 mg, 0.43 mmol, 1.00 equiv), XantPhos (50 mg, 0.09 mmol, 0.2 equiv), Cs₂CO₃ (421 mg, 1.29 mmol, 3.00 equiv) and Pd(OAc)₂ (10 mg, 0.04 mmol, 0.10 equiv) in dioxane (8 mL) was stirred at 120° C. for 1 h under N₂. LCMS indicated the reaction was completed. After cooled to room temperature, the reaction was filtered. The filtrate was concentrated to dryness under vacuum. The residue was purified by Prep-TLC (DCM:MeOH=10:1) to give 80 mg of the crude product as a white solid. The crude product was re-purified by reverse flash chromatography with the following conditions: Column: XSelect CSH Prep C18 OBD Column, 19*150 mm, 5 m; Mobile Phase A: Water (0.05% FA), Mobile Phase B: ACN; Flow rate: 25 mL/min; Gradient: 5% B to 35% B in 7 min, 35% B; Wave Length: 254 nm; RT1 (min): 5.4. The collected fraction was lyophilized to give 2-[6-(2-hydroxyethoxy)-4-{3-[(4-methyl-1,2,4-triazol-3-yl)methyl]oxetan-3-yl}pyridin-2-yl]-6-{[(3S)-3-methylpiperidin-1-yl]methyl}-4-(trifluoromethyl)-3H-isoindol-1-one (Compound 100, 52.5 mg, 19%) as a white solid. LCMS (ES, m/z): 601 [M+H]⁺; ¹H NMR (400 MHz, DMSO-d₆) δ 8.26 (s, 1H), 7.99-7.94 (m, 3H), 6.46 (s, 1H), 5.20 (s, 2H), 4.98-4.86 (s, 5H), 4.28 (t, J=4.8 Hz, 2H), 3.74 (s, 2H), 3.68 (s, 2H), 3.57 (s, 2H), 3.31 (s, 3H), 2.78-2.67 (m, 2H), 1.96-1.91 (m, 1H), 1.68-1.59 (m, 4H), 1.52-1.43 (m, 1H), 0.90-0.81 (m, 4H).

### Synthesis of Compound 101

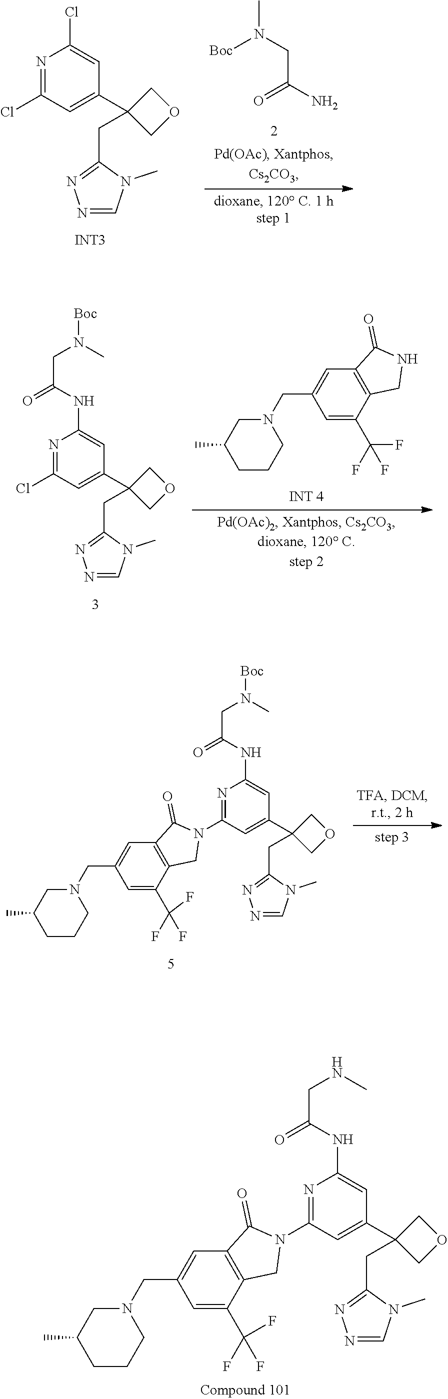

### Step 1: Synthesis of Compound 3

A solution of 2,6-dichloro-4-{3-[(4-methyl-1,2,4-triazol-3-yl)methyl]oxetan-3-yl}pyridine (INT3, 400 mg, 1.34 mmol, 1.00 equiv), tert-butyl N-(carbamoylmethyl)-N-methylcarbamate (252 mg, 1.34 mmol, 1.00 equiv), XantPhos (155 mg, 0.27 mmol, 0.20 equiv), Cs₂CO₃ (1307 mg, 4.01 mmol, 3.00 equiv) and Pd(OAc)₂ (30 mg, 0.13 mmol, 0.10 equiv) in dioxane (6 mL) was stirred at 120° C. for 1 h. LCMS indicated the reaction was completed. After cooled to room temperature. The reaction was filtered. The filtrate was concentrated to dryness under vacuum. The residue was purified by Prep-TLC (DCM:MeOH=10:1) to give tert-butyl N-{[(6-chloro-4-{3-[(4-methyl-1,2,4-triazol-3-yl)methyl]oxetan-3-yl}pyridin-2-yl)carbamoyl]methyl}-N-methylcarbamate (Compound 3, 390 mg, 63%) as a green solid. LCMS (ES, m/z): 451,453 [M+H]⁺

### Step 2: Synthesis of Compound 5

A solution of tert-butyl N-{[(6-chloro-4-{3-[(4-methyl-1,2,4-triazol-3-yl)methyl]oxetan-3-yl}pyridin-2-yl)carbamoyl]methyl}-N-methylcarbamate (Compound 3, 280 mg, 0.62 mmol, 1.00 equiv), 6-{[(3S)-3-methylpiperidin-1-yl]methyl}-4-(trifluoromethyl)-2,3-dihydroisoindol-1-one (INT4, 194 mg, 0.62 mmol, 1.00equiv), XantPhos (72 mg, 0.12 mmol, 0.20 equiv), Cs₂CO₃ (607 mg, 1.86 mmol, 3.00 equiv) and Pd(OAc)₂ (14 mg, 0.06 mmol, 0.10 equiv) in dioxane (6 mL) was stirred at 120° C. for 2 hours. LCMS indicated the reaction was completed. After cooled to room temperature. The reaction was filtered and concentrated to dryness under vacuum. The residue was purified by Prep-TLC (DCM:MeOH=10:1) to give tert-butyl N-methyl-N-{[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamoyl]methyl}carbamate (Compound 5, 200 mg, 39%) as a yellow solid. LCMS (ES, m/z): 727 [M+H]⁺

### Step 3: Synthesis of Compound 101

Tert-butyl N-methyl-N-{[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamoyl]methyl}carbamate (Compound 5, 250 mg, 0.34 mmol, 1.00 equiv) in TFA (400 uL) and DCM (4 mL) was stirred at room temperature for 2 hours. LCMS indicated the reaction was completed. The reaction was concentrated to dryness under vacuum. The residue was purified by reverse flash chromatography with the following conditions: column, C18 silica gel; mobile phase, ACN in water (0.1% FA), 0% to 60% gradient in 30 min; detector, UV 254 nm. The collected fraction was concentrated to give N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)-2-(methylamino)acetamide; formic acid (Compound 101, 100 mg, 41%) as a white solid. LCMS (ES, m/z): 627 [M+H]⁺; ¹H NMR (400 MHz, DMSO-d₆) δ8.24 (s, 1H), 8.20 (s, 2H), 8.08 (s, 1H), 7.99 (s, 1H), 7.95 (s, 1H), 7.75 (br, 1H), 5.19 (s, 2H), 4.98-4.81 (m, 4H), 3.65 (s, 2H), 3.59 (s, 2H), 3.52-3.50 (m, 2H), 3.33 (s, 3H), 2.74-2.67 (m, 2H), 2.42 (s, 3H), 1.97-1.92 (m, 1H), 1.68-1.59 (m, 4H), 1.52-1.46 (m, 1H), 0.88-0.81 (m, 4H).

### Synthesis of Compound 102

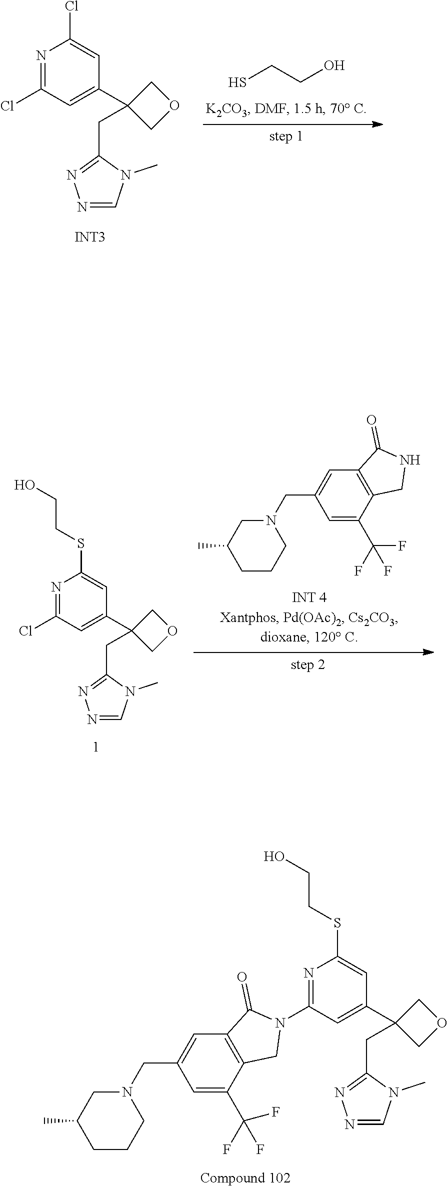

### Step 1: Synthesis of Compound 1

To a stirred mixture of 2,6-dichloro-4-{3-[(4-methyl-1,2,4-triazol-3-yl)methyl]oxetan-3-yl}pyridine (INT3, 200 mg, 0.66 mmol, 1 equiv) and β-mercaptoethanol (53 mg, 0.67 mmol, 1.01 equiv) in DMF (2 mL) was added K₂CO₃ (186 mg, 1.34 mmol, 2.01 equiv) in portions at room temperature. The resulting mixture was stirred for 2 h at 70° C. under nitrogen atmosphere. LCMS indicated the reaction was completed. The reaction mixture was filtered, and the filtrate was purified by reverse flash chromatography with the following conditions: C18 column; mobile phase, ACN in water (0.1% FA), 10% to 50% gradient in 40 min; detector, UV 254 nm. The collected fraction was concentrated under reduced pressure. This resulted in 2-[(6-chloro-4-{3-[(4-methyl-1,2,4-triazol-3-yl)methyl]oxetan-3-yl}pyridin-2-yl)sulfanyl]ethanol (Compound 1, 130 mg, 57%) as a yellow oil. LCMS:(ES·m/z):341,343[M+1]⁺.

### Step 2. Synthesis of Compound 102

To a stirred mixture of 2-[(6-chloro-4-{3-[(4-methyl-1,2,4-triazol-3-yl)methyl]oxetan-3-yl}pyridin-2-yl)sulfanyl]ethanol (Compound 1, 124 mg, 0.36 mmol, 1 equiv) and 6-{[(3S)-3-methylpiperidin-1-yl]methyl}-4-(trifluoromethyl)-2,3-dihydroisoindol-1-one (INT4, 124 mg, 0.39 mmol, 1.09 equiv) in dioxane (3 mL) was added Xantphos (42 mg, 0.073 mmol, 0.20 equiv) and Pd(OAc)₂ (8 mg, 0.036 mmol, 0.10 equiv) and Cs₂CO₃ (354 mg, 1.08 mmol, 2.99 equiv) in portions at room temperature. The resulting mixture was stirred for 1 h at room temperature under nitrogen atmosphere. LCMS indicated the reaction was completed. The reaction mixture was purified by reverse flash chromatography with the following conditions: C18 column; mobile phase, ACN in water, 10% to 60% gradient in 40 min; detector, UV 254 nm. The collected fraction was concentrated under reduced pressure. This resulted in 2-{6-[(2-hydroxyethyl)sulfanyl]-4-{3-[(4-methyl-1,2,4-triazol-3-yl)methyl]oxetan-3-yl}pyridin-2-yl}-6-{[(3S)-3-methylpiperidin-1-yl]methyl}-4-(trifluoromethyl)-3H-isoindol-1-one (Compound 102, 12.2 mg, 5.40%) as an off-white solid. LCMS:(ES·m/z):617[M+1]⁺; ¹H NMR (300 MHz, DMSO-d₆) δ 8.26 (s, 1H), 8.04 (s, 1H), 7.98 (s, 1H), 7.94 (s, 1H), 6.96 (s, 1H), 5.22 (s, 2H), 4.94-4.90 (m, 3H), 4.83-4.82 (m, 2H), 3.66-3.65 (m, 4H), 3.57 (s, 2H), 3.23-3.22 (m, 2H), 2.80-2.65 (m, 2H), 2.00-1.90 (m, 1H), 1.77-1.55 (m, 4H), 1.55-1.40 (m, 1H), 0.82-0.81 (m, 4H).

### Synthesis of Compound 103

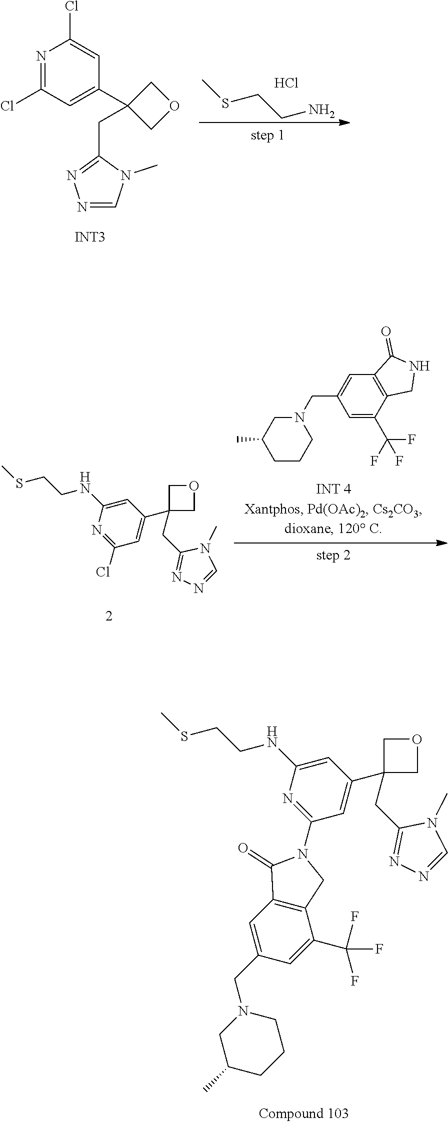

### Step 1: Synthesis of Compound 2

To a stirred mixture of 2,6-dichloro-4-{3-[(4-methyl-1,2,4-triazol-3-yl)methyl]oxetan-3-yl}pyridine (INT3, 100 mg, 0.33 mmol, 1.00 equiv) in DMF (2 mL) was added 2-(methylsulfanyl)ethanamine hydrochloride (129 mg, 1.01 mmol, 3.02 equiv) and K₂CO₃ (139 mg, 1.01 mmol, 3.0 equiv). The reaction mixture was stirred for 48 h at 100° C. under nitrogen atmosphere. ˜20% desired product could be detected by LCMS. The reaction mixture was cooled to room temperature and filtered. The filtrate was concentrated to dryness under vacuum and the residue was purified by reverse flash chromatography with the following conditions: column, C18 silica gel; mobile phase, ACN in water (0.1% FA), 10% to 60% gradient in 30 min; detector, UV 254 nm. The collected fraction was concentrated to dryness to afford 6-chloro-4-{3-[(4-methyl-1,2,4-triazol-3-yl)methyl]oxetan-3-yl}-N-[2-(methylsulfanyl)ethyl]pyridin-2-amine (Compound 2, 44 mg, 35%) as a white solid. LCMS: (ES, m/s): 354,356 [M+H]⁺.

### Step 2. Synthesis of Compound 103

To a stirred mixture of 6-chloro-4-{3-[(4-methyl-1,2,4-triazol-3-yl)methyl]oxetan-3-yl}-N-[2-(methylsulfanyl)ethyl]pyridin-2-amine (Compound 2, 38 mg, 0.11 mmol, 1 equiv) and 6-{[(3S)-3-methylpiperidin-1-yl]methyl}-4-(trifluoromethyl)-2,3-dihydroisoindol-1-one (INT4, 35 mg, 0.11 mmol, 1.05 equiv) in dioxane (400 uL) was added Cs₂CO₃ (70 mg, 0.22 mmol, 2.00 equiv), Xantphos (12 mg, 0.021 mmol, 0.20 equiv) and Pd(OAc)₂ (24 mg, 0.11 mmol, 1.00 equiv) at room temperature under nitrogen atmosphere. The resulting mixture was stirred for 1 h at 120° C. under nitrogen atmosphere. LCMS indicated the reaction was completed. The reaction mixture was cooled to room temperature and filtered. The filtrate was concentrated to dryness under vacuum and the residue was purified by Prep-HPLC with the following conditions (Column: XBridge Shield RP18 OBD Column, 19×250 mm, 10 μm; Mobile Phase A: Water (0.05% FA), Mobile Phase B: MeOH--HPLC; Flow rate: 25 mL/min; Gradient: 38% B to 48% B in 10 min, 48% B; Wave Length: 254 nm; The collected fraction was lyophilized to afford 2-(4-{3-[(4-methyl-1,2,4-triazol-3-yl)methyl]oxetan-3-yl}-6-{[2-(methylsulfanyl)ethyl]amino}pyridin-2-yl)-6-{[(3S)-3-methylpiperidin-1-yl]methyl}-4-(trifluoromethyl)-3H-isoindol-1-one (Compound 103, 8.1 mg, 11%) as a yellow solid. LCMS:(ES, m/z): 630[M+1]⁺; ¹H-NMR (300 MHz, DMSO-d₆) 8.25 (s, 1H), 7.95-7.91 (m, 2H), 7.45 (s, 1H), 6.89 (t, J=5.7 Hz, 1H), 5.95 (s, 1H), 5.17 (s, 2H), 4.90 (d, J=4.5 Hz, 2H), 4.78 (d, J=4.5 Hz, 2H), 3.64 (s, 2H), 3.56-3.46 (m, 4H), 3.23 (s, 3H), 2.73-2.67 (m, 2H), 2.65-2.61 (m, 2H), 2.13 (s, 3H), 1.96-1.93 (m, 1H), 1.67-1.58 (m, 4H), 1.49-1.46 (m, 1H), 0.87-0.81 (m, 4H).

### Synthesis of Compound 104

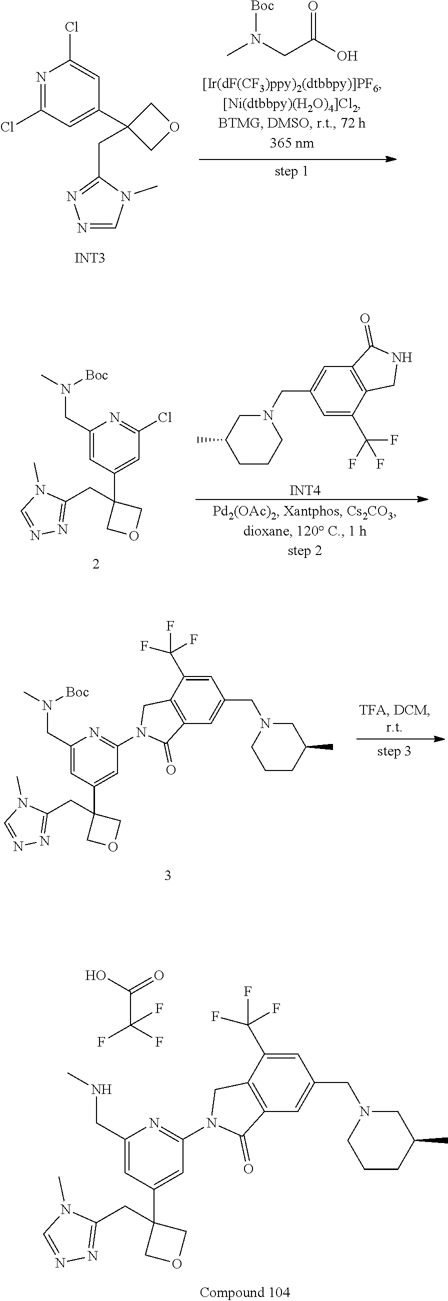

### Step 1. Synthesis of Compound 2

To a stirred mixture of 2,6-dichloro-4-{3-[(4-methyl-1,2,4-triazol-3-yl)methyl]oxetan-3-yl}pyridine (INT3, 200 mg, 0.66 mmol, 1 equiv) and [(tert-butoxycarbonyl)(methyl)amino]acetic acid (190 mg, 1.00 mmol, 1.50 equiv) in DMSO (12 mL) were added [Ir(dF(CF₃)ppy)₂ (dtbbpy)]PF₆ (150 mg, 0.13 mmol, 0.20 equiv) and [Ni(dtbbpy)(H₂O)₄]Cl₂ (60 mg, 0.13 mmol, 0.19 equiv) in portions and BTMG (170 mg, 0.99 mmol, 1.49 equiv) dropwise at room temperature under nitrogen atmosphere. The resulting mixture was stirred and irradiated using blue LED lamp (365 nm) for overnight at room temperature. LCMS indicated the reaction was 50% 2,6-dichloro-4-{3-[(4-methyl-1,2,4-triazol-3-yl)methyl]oxetan-3-yl}pyridine and 50% desired product. The reaction mixture was purified by reverse flash chromatography with the following conditions: C18 column; mobile phase, ACN in water (0.1% FA), 10% to 80% gradient in 40 min; detector, UV 254 nm. The resulting fraction was concentrated under reduced pressure. This resulted in tert-butyl N-[(6-chloro-4-{3-[(4-methyl-1,2,4-triazol-3-yl)methyl]oxetan-3-yl}pyridin-2-yl)methyl]-N-methylcarbamate (Compound 2, 68 mg, 24%) as a yellow oil. LCMS:(ES·m/z):408,410[M+1]⁺.

### Step 2: Synthesis of Compound 3

To a stirred mixture of tert-butyl N-[(6-chloro-4-{3-[(4-methyl-1,2,4-triazol-3-yl)methyl]oxetan-3-yl}pyridin-2-yl)methyl]-N-methylcarbamate (Compound 2, 63 mg, 0.15 mmol, 1 equiv) and 6-{[(3S)-3-methylpiperidin-1-yl]methyl}-4-(trifluoromethyl)-2,3-dihydroisoindol-1-one (INT4, 50 mg, 0.16 mmol, 1.04 equiv) in dioxane (0.5 mL) was added Pd(OAc)₂ (4 mg, 0.018 mmol, 0.12 equiv) and Xantphos (18 mg, 0.03 mmol, 0.20 equiv) and Cs₂CO₃ (152 mg, 0.47 mmol, 3.02 equiv) in portions at room temperature. The resulting mixture was stirred for overnight at 120° C. under nitrogen atmosphere. After cooled to room temperature, the reaction mixture was filtered, and the filtrate was concentrated to dryness under reduced pressure. The residue was purified by reverse flash chromatography with the following conditions: C18 column; mobile phase, ACN in water (0.1% FA), 10% to 50% gradient in 40 min; detector, UV 254 nm. The resulting mixture was concentrated under vacuum. The crude product (37 mg) was re-purified by Prep-HPLC with the following conditions (Column: XBridge Shield RP18 OBD Column, 30*150 mm, 5 m; Mobile Phase A: Water (10 mmol/L NH₄HCO₃+0.1% NH₃·H₂O), Mobile Phase B: ACN; Flow rate: 60 mL/min; Gradient: 33% B to 56% B in 7 min, 56% B; Wave Length: 254 nm; The collected fraction was concentrated to afford tert-butyl N-methyl-N-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)methyl]carbamate (Compound 3, 12 mg, 11%) as an off-white solid. LCMS:(ES·m/z):684[M+1]⁺.

### Step 3: Synthesis of Compound 104

To a stirred mixture of tert-butyl N-methyl-N-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)methyl]carbamate (12 mg, 0.018 mmol, 1 equiv) in DCM (1200 uL) was added TFA (300 uL) dropwise at room temperature. The resulting mixture was stirred for 1 h at room temperature under air atmosphere. LCMS indicated the reaction was completed. The reaction mixture was concentrated to dryness under vacuum. The resulting solid was dried by lyophilization. This resulted in 2-(4-{3-[(4-methyl-1,2,4-triazol-3-yl)methyl]oxetan-3-yl}-6-[(methylamino)methyl]pyridin-2-yl)-6-{[(3S)-3-methylpiperidin-1-yl]methyl}-4-(trifluoromethyl)-3H-isoindol-1-one; trifluoroacetic acid (9.0 mg, 69%) as a colorless oil. LCMS:(ES·m/z):584[M+1]⁺; ¹H NMR (400 MHz, CD₃OD) δ 8.87 (s, 1H), 8.48 (s, 1H), 8.35 (s, 1H), 8.28 (s, 1H), 7.27 (s, 1H), 5.46 (s, 2H), 5.07-5.02 (m, 4H), 4.52 (s, 2H), 4.44 (s, 2H), 3.96 (s, 2H), 3.85 (s, 3H), 3.77-3.76 (m, 1H), 3.63-3.62 (m, 1H), 2.97-2.95 (m, 1H), 2.89 (s, 3H), 2.85-2.71 (m, 1H), 2.00-1.61 (m, 4H), 1.35-1.29 (m, 1H), 1.26-1.10 (m, 1H), 1.00 (s, 3H).

### Synthesis of Compound 105

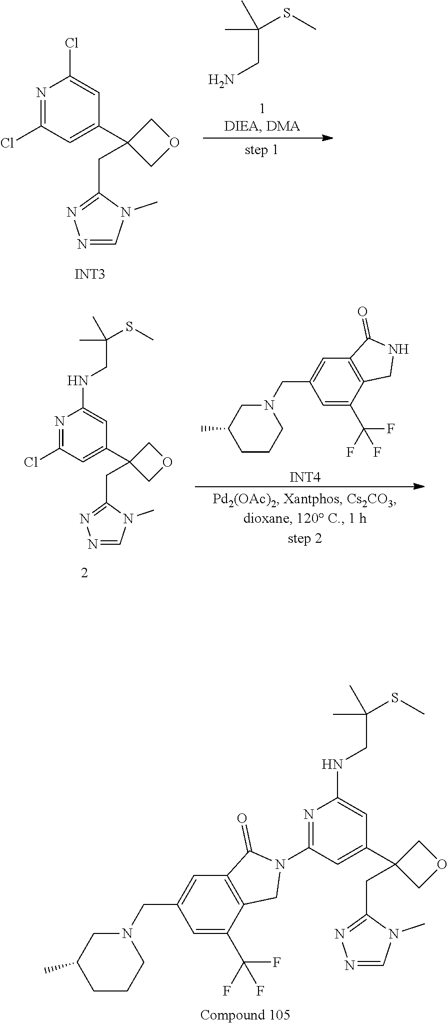

### Step 1: Synthesis of Compound 2

To a stirred solution of 2,6-dichloro-4-{3-[(4-methyl-1,2,4-triazol-3-yl)methyl]oxetan-3-yl}pyridine (INT3, 100 mg, 0.34 mmol, 1.0 equiv) and 2-methyl-2-(methylsulfanyl)propan-1-amine (48 mg, 0.40 mmol, 1.2 equiv) in DMA (2.0 mL) was added DIEA (172 mg, 1.33 mmol, 4.00 equiv) in portions at room temperature under nitrogen atmosphere. The resulting mixture was stirred for 4d at 80° C. under nitrogen atmosphere. ˜30% desired product could be detected by LCMS. The reaction mixture was purified by reverse flash chromatography with the following conditions: column, C18 silica gel; mobile phase, ACN in water (0.1% FA), 10% to 60% gradient in 30 min; detector, UV 254 nm. The resulting mixture was concentrated under vacuum to afford6-chloro-4-{3-[(4-methyl-1,2,4-triazol-3-yl)methyl]oxetan-3-yl}-N-[2-methyl-2-(methylsulfanyl)propyl]pyridin-2-amine (Compound 2, 17 mg, 13%) as a solid. LCMS: (ES, m/s): 382,384 [M+H]⁺.

### Step 2: Synthesis Compound 105

To a stirred solution of 6-chloro-4-{3-[(4-methyl-1,2,4-triazol-3-yl)methyl]oxetan-3-yl}-N-[2-methyl-2-(methylsulfanyl)propyl]pyridin-2-amine (Compound 2, 17 mg, 0.04 mmol, 1.0 equiv) and 6-{[(3S)-3-methylpiperidin-1-yl]methyl}-4-(trifluoromethyl)-2,3-dihydroisoindol-1-one (INT4, 14 mg, 0.04 mmol, 1.0 equiv) in dioxane (1.5 mL) was added Xantphos (10 mg, 0.016 mmol, 0.4 equiv), Pd(OAc)₂ (4 mg, 0.016 mmol, 0.4 equiv) and Cs₂CO₃ (43 mg, 0.13 mmol, 3.0 equiv) in portions at room temperature under nitrogen atmosphere. The resulting mixture was stirred for 1 h at 120° C. under nitrogen atmosphere. LCMS indicated the reaction was completed. The reaction mixture was purified by Prep-HPLC with the following conditions (Column: XBridge Shield RP18 OBD Column, 30*150 mm, 5 m; Mobile Phase A: Water (0.1% FA), Mobile Phase B: ACN; Flow rate: 60 mL/min; Gradient: 23% B to 43% B in 7 min, 43% B; Wave Length: 254 nm; The collected fraction was lyophilized to afford 2-(4-{3-[(4-methyl-1,2,4-triazol-3-yl)methyl]oxetan-3-yl}-6-{[2-methyl-2-(methylsulfanyl)propyl]amino}pyridin-2-yl)-6-{[(3S)-3-methylpiperidin-1-yl]methyl}-4-(trifluoromethyl)-3H-isoindol-1-one; formic acid (Compound 105, 2.6 mg, 7%) as a white solid. LCMS: (ES, m/s): 658 [M+H]⁺; ¹H-NMR (300 MHz, DMSO-d₆) 8.49 (br s, 1H), 8.24 (s, 1H), 7.95-7.91 (m, 2H), 7.39 (s, 1H), 6.78 (t, J=5.4 Hz, 1H), 6.15 (s, 1H), 5.16 (s, 2H), 4.96-4.83 (m, 2H), 4.81-4.77 (m, 2H), 3.62 (d, J=6.9 Hz, 2H), 3.56-3.48 (m, 4H), 3.20 (s, 3H), 2.70-2.68 (m, 2H), 2.06 (s, 3H), 1.99-1.92 (m, 1H), 1.70-1.40 (m, 5H), 1.28-1.15 (m, 6H), 0.95-0.75 (m, 4H).

### Synthesis of Compound 106

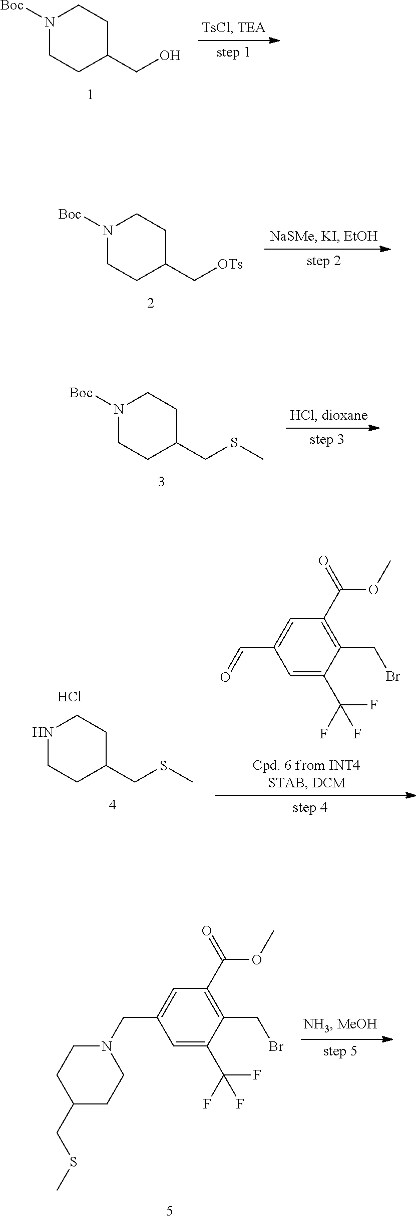

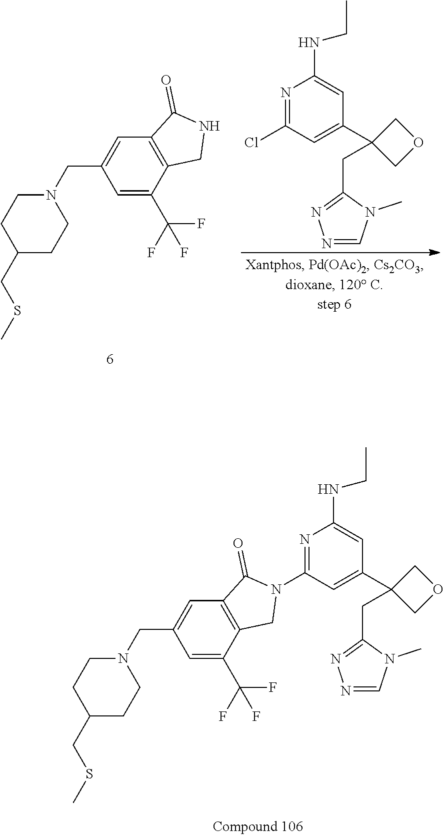 [SMILES: CSCC1CCN(Cc2cc3c(c(C(F)(F)F)c2)CNC3=O)CC1 ; CCNc1cc(C2(Cc3nncn3C)COC2)cc(Cl)n1 ; CCNc1cc(C2(Cc3nncn3C)COC2)cc(N2Cc3c(cc(CN4CCC(CSC)CC4)cc3C(F)(F)F)C2=O)n1]

### Step 1: Synthesis of Compound 2

To a stirred solution of tert-butyl 4-(hydroxymethyl)piperidine-1-carboxylate (Compound 1, 10 g, 46.44 mmol, 1 equiv) in DCM (250 mL) was added TsCl (17.71 g, 92.89 mmol, 2 equiv) and TEA (14.10 g, 139.34 mmol, 3 equiv) at 0° C. The resulting mixture was stirred for overnight at room temperature under nitrogen atmosphere. LCMS indicated the reaction was completed. The reaction was quenched with MeOH at 0° C. The resulting mixture was concentrated under vacuum. The residue was purified by silica gel column chromatography, eluted with PE/EA (5:1) to afford tert-butyl 4-{[(4-methylbenzenesulfonyl)oxy]methyl}piperidine-1-carboxylate (Compound 2, 12 g, 69%) as an off-white solid. LCMS (ESI, m/z):370[M+H]⁺

### Step 2: Synthesis of Compound 3

To a stirred mixture of tert-butyl 4-{[(4-methylbenzenesulfonyl)oxy]methyl}piperidine-1-carboxylate (Compound 2, 5 g, 13.53 mmol, 1 equiv) and KI (2.25 g, 13.53 mmol, 1 equiv) in EtOH (50 mL) was added sodium thiomethoxide (1.90 g, 27.06 mmol, 2 equiv) at room temperature. The resulting mixture was stirred for 1 h at 80° C. under nitrogen atmosphere. LCMS indicated the reaction was completed. The mixture was allowed to cool down to room temperature. The reaction was quenched with Water at room temperature. The resulting mixture was extracted with CH₂Cl₂ (3×100 mL). The combined organic layers were washed with brine (100 mL), dried over anhydrous Na₂SO₄. After filtration, the filtrate was concentrated under reduced pressure. This resulted in tert-butyl 4-[(methylsulfanyl)methyl]piperidine-1-carboxylate (Compound 3, 2.8 g, 84%) as an off-white solid. LCMS (ESI, m/z):246[M+H]⁺.

### Step 3: Synthesis of Compound 4

The solution of tert-butyl 4-[(methylsulfanyl)methyl]piperidine-1-carboxylate (Compound 3, 560 mg, 2.28 mmol, 1 equiv) in HCl (g, 4N in MeOH, 5.6 mL) was stirred for 1 h at room temperature under nitrogen atmosphere. LCMS indicated the reaction was completed. The resulting mixture was concentrated under reduced pressure. The crude product was used in the next step directly without further purification. This resulted in 4-[(methylsulfanyl)methyl]piperidine hydrochloride (370 mg, 89%) as a yellow solid. LCMS (ESI, m/z):146[M+H−HCl]⁺

### Step 4: Synthesis of Compound 5

To a stirred solution of 4-[(methylsulfanyl)methyl]piperidine hydrochloride (Compound 4, 350 mg, 1.92 mmol, 1 equiv) in DCM (4 mL) was added methyl 2-(bromomethyl)-5-formyl-3-(trifluoromethyl)benzoate (Compound 6 from the procedure for preparing INT4, 626 mg, 1.92 mmol, 1 equiv) at room temperature. The resulting mixture was stirred for 30 min at room temperature under nitrogen atmosphere. To the above mixture was added STAB (1.63 g, 7.70 mmol, 4 equiv) in portions at room temperature. The resulting mixture was stirred for additional overnight at room temperature. LCMS indicated the reaction was completed. The reaction was quenched with MeOH at 0° C. and stirred for 30 min. The resulting mixture was concentrated under reduced pressure. The residue was purified by silica gel column chromatography, eluted with CH₂Cl₂/MeOH (10:1) to afford methyl 2-(bromomethyl)-5-({4-[(methylsulfanyl)methyl]piperidin-1-yl}methyl)-3-(trifluoromethyl)benzoate (Compound 5, 820 mg, 93%) as a yellow oil. LCMS (ESI, m/z):454,456[M+H].

### Step 5: Synthesis of Compound 6

To a stirred solution of methyl 2-(bromomethyl)-5-({4-[(methylsulfanyl)methyl]piperidin-1-yl}methyl)-3-(trifluoromethyl)benzoate (Compound 5, 400 mg, 0.88 mmol, 1 equiv) in NH₃ (g, 7N in MeOH, 4 mL) at room temperature. The resulting mixture was stirred for 1 h at room temperature under nitrogen atmosphere. LCMS indicated the reaction was completed. The resulting mixture was concentrated under reduced pressure. The residue was purified by silica gel column chromatography, eluted with CH₂Cl₂/MeOH (14:1) to afford 6-({4-[(methylsulfanyl)methyl]piperidin-1-yl}methyl)-4-(trifluoromethyl)-2,3-dihydroisoindol-1-one (Compound 6, 170 mg, 53%) as a white solid. LCMS (ESI, m/z):359[M+H]⁺.

### Step 6: Synthesis of Compound 106

To a stirred solution of 6-({4-[(methylsulfanyl)methyl]piperidin-1-yl}methyl)-4-(trifluoromethyl)-2,3-dihydroisoindol-1-one (Compound 6, 86 mg, 0.24 mmol, 1 equiv) and 6-chloro-N-ethyl-4-{3-[(4-methyl-1,2,4-triazol-3-yl)methyl]oxetan-3-yl}pyridin-2-amine (prepared as described in WO2020210508, 73 mg, 0.24 mmol, 1 equiv) in dioxane (2 mL) were added Cs₂CO₃ (156 mg, 0.48 mmol, 2 equiv), di-tert-butyl([2-[2,4,6-tris(propan-2-yl)phenyl]phenyl])phosphane (10 mg, 0.02 mmol, 0.1 equiv) and tBuXPhos Pd G3 (19 mg, 0.02 mmol, 0.1 equiv) at room temperature under nitrogen atmosphere. The resulting mixture was stirred for overnight at 120° C. under nitrogen atmosphere. LCMS showed 45% product. The reaction mixture was cooled down to room temperature. The reaction mixture was purified by reverse flash chromatography with the following conditions: column, C18 silica gel; mobile phase, ACN in water (0.1% FA), 5% to 60% gradient in 30 min; detector, UV 254 nm. The collected fraction was concentrated to afford crude product (40 mg) as a white solid. The crude product (40 mg) was purified by Prep-HPLC with the following conditions (Column: XBridge Shield RP18 OBD Column, 30*150 mm, 5 m; Mobile Phase A: Water (0.1% FA), Mobile Phase B: ACN; Flow rate: 60 mL/min; Gradient: 19% B to 39% B in 7 min, 39% B; Wave Length: 254 nm; RT1 (min): 4.62; The collected fraction was lyophilized to afford 2-[6-(ethylamino)-4-{3-[(4-methyl-1,2,4-triazol-3-yl)methyl]oxetan-3-yl}pyridin-2-yl]-6-({4-[(methylsulfanyl)methyl]piperidin-1-yl}methyl)-4-(trifluoromethyl)-3H-isoindol-1-one (10.8 mg, 7%) as a white solid. LCMS (ESI, ms):630[M+H]⁺; ¹H NMR (400 MHz, DMSO-d₆) δ 8.38 (s, 1H), 7.94-7.90 (m, 2H), 7.40 (s, 1H), 6.62 (t, J=5.2 Hz, 1H), 5.89 (s, 1H), 5.14 (s, 2H), 4.90 (d, J=6.0 Hz, 2H), 4.78 (d, J=6.0 Hz, 2H), 3.65 (s, 2H), 3.49 (s, 2H), 3.24-3.17 (m, 5H), 2.82-2.79 (m, 2H), 2.41 (d, J=6.8 Hz, 2H), 2.02-1.96 (m, 5H), 1.75-1.72 (m, 2H), 1.46-1.44 (m, 1H), 1.26-1.10 (m, 5H).

### Synthesis of Compound 107

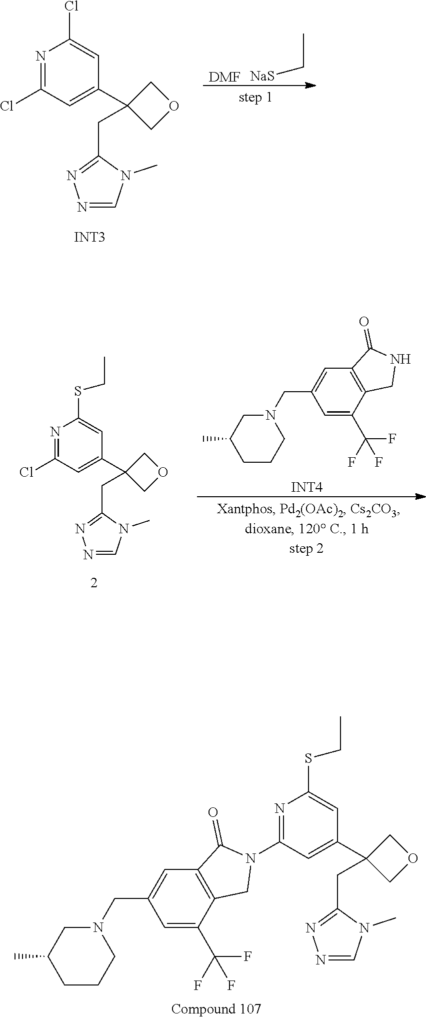

### Step 1: Synthesis of Compound 2

To a stirred solution of 2,6-dichloro-4-{3-[(4-methyl-1,2,4-triazol-3-yl)methyl]oxetan-3-yl}pyridine (INT3, 2 g, 6.68 mmol, 1 equiv) in DMF (20 mL) was added (ethylsulfanyl)sodium (0.62 g, 7.35 mmol, 1.1 equiv) in portions at room temperature. The resulting mixture was stirred for 2 h at 50° C. under nitrogen atmosphere. LCMS indicated the reaction was completed. The resulting mixture was diluted with water (100 mL) and extracted with EtOAc (3×100 mL). The combined organic layers were washed with brine (3×100 mL), dried over anhydrous Na₂SO₄. After filtration, the filtrate was concentrated under reduced pressure. The residue was purified by reverse flash chromatography with the following conditions: column, C18 silica gel; mobile phase, FA in water, 0% to 50% gradient in 40 min; detector, UV 254 nm. This resulted in 2-chloro-6-(ethylsulfanyl)-4-{3-[(4-methyl-1,2,4-triazol-3-yl)methyl]oxetan-3-yl}pyridine (Compound 2, 1.4 g, 62%) as a yellow solid. LCMS:(ES·m/z): 325,327[M+1]⁺.

### Step 2: Synthesis of Compound 107

To a stirred mixture of 2-chloro-6-(ethylsulfanyl)-4-{3-[(4-methyl-1,2,4-triazol-3-yl)methyl]oxetan-3-yl}pyridine (Compound 2, 120 mg, 0.36 mmol, 1.20 equiv) and 6-{[(3S)-3-methylpiperidin-1-yl]methyl}-4-(trifluoromethyl)-2,3-dihydroisoindol-1-one (INT4, 96 mg, 0.30 mmol, 1.00 equiv) in dioxane (5 mL) was added Cs₂CO₃ (200 mg, 0.61 mmol, 2.00 equiv) and Xantphos (35 mg, 0.06 mmol, 0.20 equiv) and Pd(OAc)₂ (6.90 mg, 0.03 mmol, 0.10 equiv) in portions at room temperature. The resulting mixture was stirred for 2 h at 120° C. under nitrogen atmosphere. The mixture was allowed to cool down to room temperature. LCMS indicated the reaction was completed. The resulting mixture was filtered, the filter cake was washed with DCM (3×10 mL). The filtrate was concentrated under reduced pressure. The residue was purified by silica gel column chromatography, eluted with DCM/MeOH (10:1) to afford 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-4-(trifluoromethyl)-3H-isoindol-1-one (17.7 mg, 9%) as a white solid. LCMS:(ES·m/z):601[M+1]⁺; ¹H NMR (400 MHz, DMSO-d₆) δ8.26 (s, 1H), 8.05-7.94 (m, 3H), 6.91 (d, J=1.2 Hz, 1H), 5.23 (s, 2H), 4.92-4.82 (m, 4H), 3.63-3.57 (m, 4H), 3.34-3.30 (m, 5H), 3.14-3.12 (m, 2H), 2.74-2.68 (m, 2H), 1.98-1.90 (m, 1H), 1.65-1.62 (m, 4H), 1.50-1.45 (m, 1H), 1.35-1.32 (m, 3H), 0.82-0.81 (m, 4H).

### Synthesis of Compound 108

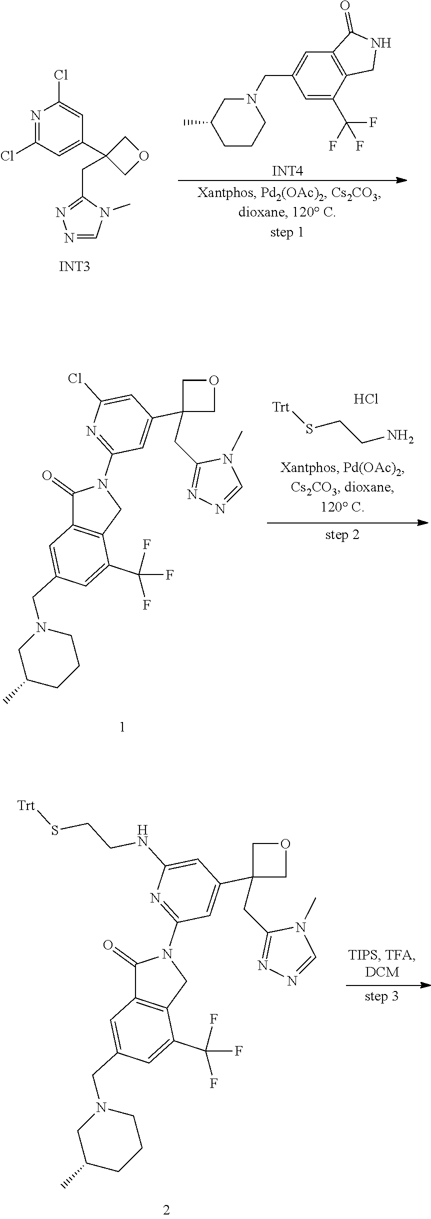

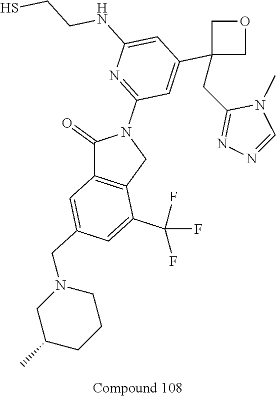 [SMILES: C[C@H]1CCCN(Cc2cc3c(c(C(F)(F)F)c2)CN(c2cc(C4(Cc5nncn5C)COC4)cc(NCCS)n2)C3=O)C1]

### Step 1: Synthesis of Compound 1

To a stirred solution of 2,6-dichloro-4-{3-[(4-methyl-1,2,4-triazol-3-yl)methyl]oxetan-3-yl}pyridine (INT3, 300 mg, 1.00 mmol, 1 equiv) and 6-{[(3S)-3-methylpiperidin-1-yl]methyl}-4-(trifluoromethyl)-2,3-dihydroisoindol-1-one (INT4, 328 mg, 1.05 mmol, 1.05 equiv) in dioxane (6 mL) were added Cs₂CO₃ (653 mg, 2.00 mmol, 2 equiv), XantPhos (116 mg, 0.20 mmol, 0.2 equiv) and Pd(OAc)₂ (22 mg, 0.10 mmol, 0.1 equiv) at room temperature under nitrogen atmosphere. The resulting mixture was stirred for 1 h at 120° C. under nitrogen atmosphere. Desired product could be detected by LCMS. The mixture was allowed to cool down to room temperature. The resulting mixture was concentrated under vacuum. The residue was purified by silica gel column chromatography, eluted with CH₂Cl₂/MeOH (10:1) to afford 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(trifluoromethyl)-3H-isoindol-1-one (Compound 1, 250 mg, 43%) as a white solid. LCMS (ESI, ms):575,577[M+H]⁺.

### Step 2: Synthesis of Compound 2

To a stirred solution of 2-(6-chloro-4-{2-[(4-methyl-1,2,4-triazol-3-yl)methyl]oxiran-2-yl}pyridin-2-yl)-6-{[(3S)-3-methylpiperidin-1-yl]methyl}-4-(trifluoromethyl)-3H-isoindol-1-one (Compound 1, 250 mg, 0.44 mmol, 1 equiv) and 2-[(triphenylmethyl)sulfanyl]ethanamine hydrochloride (190 mg, 0.53 mmol, 1.2 equiv) in dioxane (3 mL) were added Cs₂CO₃ (290 mg, 0.89 mmol, 2 equiv), XantPhos (51 mg, 0.08 mmol, 0.2 equiv) and Pd(OAc)₂ (10 mg, 0.04 mmol, 0.1 equiv) at room temperature under nitrogen atmosphere. The resulting mixture was stirred for 2 h at 120° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (10:1) to afford 2-(4-{3-[(4-methyl-1,2,4-triazol-3-yl)methyl]oxetan-3-yl}-6-({2-[(triphenylmethyl)sulfanyl]ethyl}amino)pyridin-2-yl)-6-{[(3S)-3-methylpiperidin-1-yl]methyl}-4-(trifluoromethyl)-3H-isoindol-1-one (Compound 2, 165 mg, 43%) as a yellow solid. LCMS (ESI, m/z):858[M+H]⁺.

### Step 3: Synthesis of Compound 108

To a stirred solution of 2-(4-{3-[(4-methyl-1,2,4-triazol-3-yl)methyl]oxetan-3-yl}-6-({2-[(triphenylmethyl)sulfanyl]ethyl}amino)pyridin-2-yl)-6-{[(3S)-3-methylpiperidin-1-yl]methyl}-4-(trifluoromethyl)-3H-isoindol-1-one (165 mg, 0.19 mmol, 1 equiv) and TFA (2 mL) in DCM (4 mL) was added tris(propan-2-yl)silane (200 uL) at room temperature under nitrogen atmosphere. The resulting mixture was stirred for 1 h at room temperature under nitrogen atmosphere. LCMS indicated the reaction was completed. The resulting mixture was concentrated under reduced pressure. The residue was purified by reverse flash chromatography with the following conditions: column, silica gel; mobile phase, MeCN in water (0.1% FA), 5% to 60% gradient in 30 min; detector, UV 254 nm. The collected fraction was lyophilized to afford 2-(4-{3-[(4-methyl-1,2,4-triazol-3-yl)methyl]oxetan-3-yl}-6-[(2-sulfanylethyl)amino]pyridin-2-yl)-6-{[(3S)-3-methylpiperidin-1-yl]methyl}-4-(trifluoromethyl)-3H-isoindol-1-one (Compound 108, 47.9 mg, 37%) as a white solid. LCMS:(ES·m/z): 616[M+H]⁺; ¹H NMR (300 MHz, DMSO-d₆) δ 8.29-8.23 (m, 1H), 8.01-7.83 (m, 2H), 7.44 (s, 1H), 6.91 (t, J=5.7 Hz, 1H), 5.97 (s, 1H), 5.24 (s, 2H), 4.97-4.84 (m, 2H), 4.79-4.74 (m, 2H), 3.79-3.77 (m, 2H), 3.49 (s, 2H), 3.41 (s, 2H), 3.23 (s, 3H), 2.73-2.63 (m, 4H), 2.26 (t, J=7.8 Hz, 1H), 1.98-1.95 (m, 1H), 1.63-1.50 (m, 6H), 0.91-0.81 (m, 4H).

### Synthesis of Compound 109

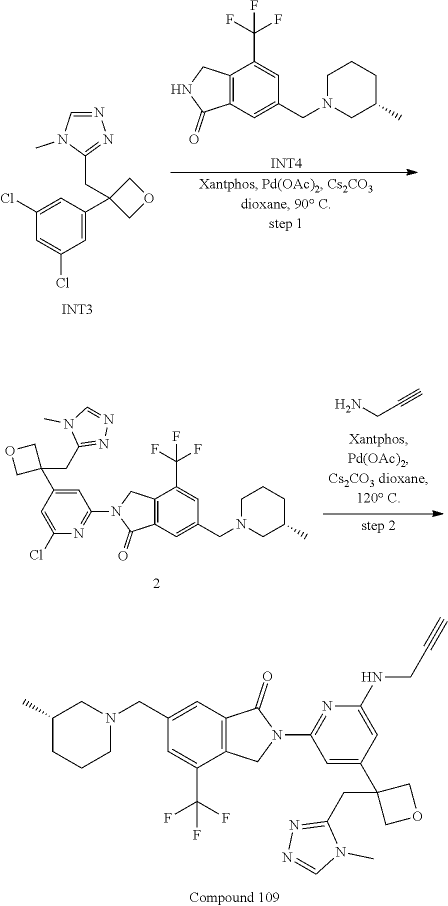

### Step 1: Synthesis of Compound 2

To a stirred mixture of 2,6-dichloro-4-(3-((4-methyl-4H-1,2,4-triazol-3-yl)methyl)oxetan-3-yl)pyridine (INT3, 235 mg, 0.786 mmol, 1 equiv) and (S)-6-((3-methylpiperidin-1-yl)methyl)-4-(trifluoromethyl)isoindolin-1-one (INT4, 245 mg, 0.786 mmol, 1 equiv) in dioxane (7 mL) was added Cs₂CO₃ (512 mg, 1.57 mmol, 2 equiv) and Xantphos (90.8 mg, 0.157 mmol, 0.2 equiv) and Pd(OAc)₂ (17.6 mg, 0.0786 mmol, 0.1 equiv) in portions at room temperature. The resulting mixture was stirred for 1 h at 90° C. under nitrogen atmosphere. The mixture was allowed to cool down to room temperature. LCMS indicated the reaction was completed. The reaction was quenched with saturated sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (9:1) to afford (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 2, 300 mg, 66.4%) as a green solid. LCMS:(ES·m/z):575,577[M+1]⁺.

### Step 2: Synthesis of Compound 109

To a stirred mixture of (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 2, 30 mg, 0.052 mmol, 1 equiv) and propargylamine (5.7 mg, 0.10 mmol, 2 equiv) in dioxane (1 mL) was added Cs₂CO₃ (113 mg, 0.34 mmol, 2 equiv) and Xantphos (6.0 mg, 0.01 mmol, 0.2 equiv) and Pd(OAc)₂ (1.2 mg, 0.05 mmol, 0.1 equiv) in portions at room temperature. The resulting mixture was stirred for 40 min at 120° C. under nitrogen atmosphere. LCMS indicated the reaction was completed. The resulting mixture was filtered and loaded at column as is. Column was dried in vacuum and purified by silica gel column chromatography, eluted with CH₂Cl₂/MeOH (3:1) to afford (S)-2-(4-(3-((4-methyl-4H-1,2,4-triazol-3-yl)methyl)oxetan-3-yl)-6-(prop-2-yn-1-ylamino)pyridin-2-yl)-6-((3-methylpiperidin-1-yl)methyl)-4-(trifluoromethyl)isoindolin-1-one(Compound 109, 13 mg, 42%) as a yellow solid. LCMS:(ES·m/z):594[M+1]⁺; ¹H NMR (400 MHz, CDCl₃) δ 8.30 (d, J=1.5 Hz, 1H), 8.02 (s, 1H), 7.94 (s, 1H), 7.86 (s, 1H), 6.97 (d, J=1.5 Hz, 1H), 5.25-5.20 (m, 2H), 5.11 (d, J=6.4 Hz, 2H), 5.05 (d, J=6.4 Hz, 2H), 3.69 (s, 2H), 3.64 (d, J=7.3 Hz, 1H), 3.60 (s, 4H), 3.26 (s, 3H), 2.75 (dd, J=17.3, 9.8 Hz, 2H), 1.95 (td, J=11.1, 3.3 Hz, 1H), 1.47 (t, J=7.2 Hz, 2H), 1.25 (s, 8H), 1.07 (s, 1H), 0.92-0.82 (m, 9H), 0.07 (s, 2H).

### Synthesis of Compound 110

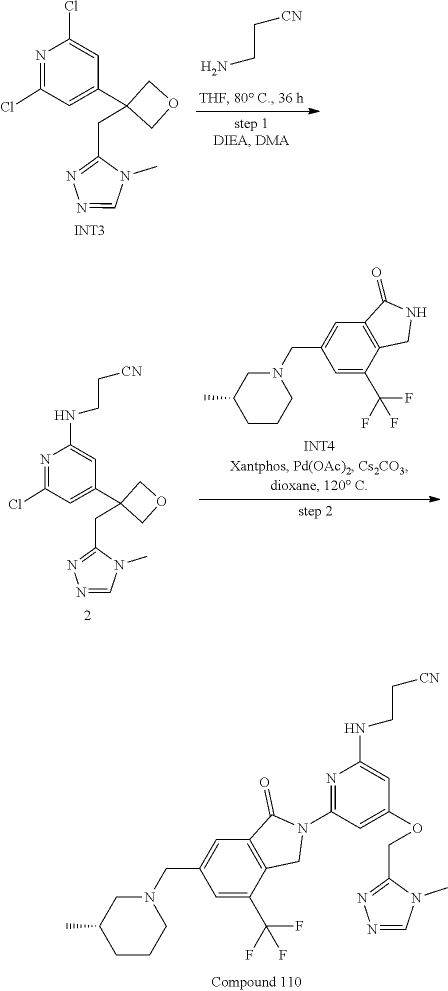

### Step 1: Synthesis of Compound 2

To a stirred solution of 2,6-dichloro-4-{3-[(4-methyl-1,2,4-triazol-3-yl)methyl]oxetan-3-yl}pyridine (INT3, 1.0 g, 3.34 mmol, 1.0 equiv) and β-aminopropionitrile (4.69 g, 66.90 mmol, 20.0 equiv) in DMA (4.0 mL) was added K₂CO₃ (0.92 g, 6.68 mmol, 2.0 equiv) in portion at room temperature under nitrogen atmosphere. The resulting mixture was stirred for two days at 120° C. under nitrogen atmosphere. ˜70% desired product could be by LCMS. The reaction was cooled to room temperature and quenched with water at room temperature. The aqueous layer was extracted with CH₂Cl₂ (3×100 mL). The resulting mixture was concentrated under vacuum. The residue was purified by silica gel column chromatography, eluted with CH₂Cl₂/MeOH (5:1) to afford 3-[(6-chloro-4-{3-[(4-methyl-1,2,4-triazol-3-yl)methyl]oxetan-3-yl}pyridin-2-yl)amino]propanenitrile (600 mg, 49%) as a light green solid. LCMS: (ES, m/s): 333,335 [M+H]⁺.

### Step 2: Synthesis of Compound 110

To a stirred solution of 6-{[(3S)-3-methylpiperidin-1-yl]methyl}-4-(trifluoromethyl)-2,3-dihydroisoindol-1-one (Compound 2, 47 mg, 0.15 mmol, 1.0 equiv) and 3-[(6-chloro-4-{3-[(4-methyl-1,2,4-triazol-3-yl)methyl]oxetan-3-yl}pyridin-2-yl)amino]propanenitrile (INT4, 50 mg, 0.15 mmol, 1.0 equiv) in dioxane (2.0 mL) was added Xantphos (34 mg, 0.06 mmol, 0.4 equiv), Pd(OAc)₂ (13 mg, 0.06 mmol, 0.4 equiv) and Cs₂CO₃ (146 mg, 0.45 mmol, 3.0 equiv) in portions at room temperature under nitrogen atmosphere. The resulting mixture was stirred for 1 h at 120° C. under nitrogen atmosphere. LCMS indicated the reaction was completed. The resulting mixture was filtered, the filter cake was washed with ACN and DCM. The filtrate was concentrated under reduced pressure. The residue was purified by silica gel column chromatography, eluted with CH₂Cl₂/MeOH (5:1) to afford the crude product. The crude product (50 mg) was purified by Prep-HPLC with the following conditions (Column: Mobile Phase A: Water (0.1% FA), Mobile Phase B: ACN). The collected fraction was lyophilized to afford 3-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]propanenitrile; formic acid (Compound 110, 25 mg, 25%) as a white solid. LCMS: (ES, m/s): 609 [M+H]⁺, 305 [M/2+H]⁺; ¹H NMR (300 MHz, DMSO-d₆) δ 8.24 (s, 1H), 7.95-7.91 (m, 2H), 7.49 (s, 1H), 7.07 (t, J=5.4 Hz, 1H), 6.05 (s, 1H), 5.20 (s, 2H), 4.91 (d, J=6 Hz, 2H), 4.78 (d, J=6 Hz, 2H), 3.64 (s, 2H), 3.53-3.50 (m, 4H), 3.25 (s, 3H), 2.81 (t, J=6.3 Hz, 2H), 2.72 (t, J=7.4 Hz, 2H), 1.97-1.93 (m, 1H), 1.69-1.42 (m, 5H), 0.95-0.78 (m, 4H).

### Synthesis of Compound 111

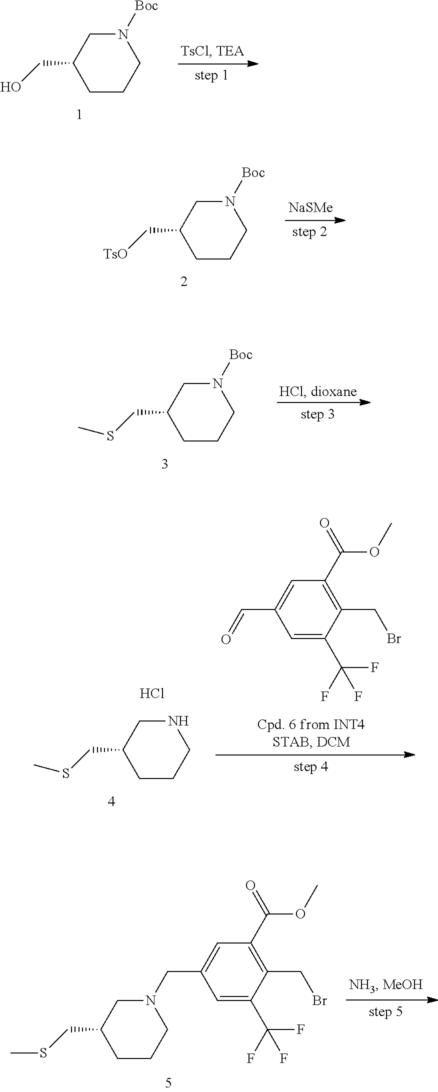

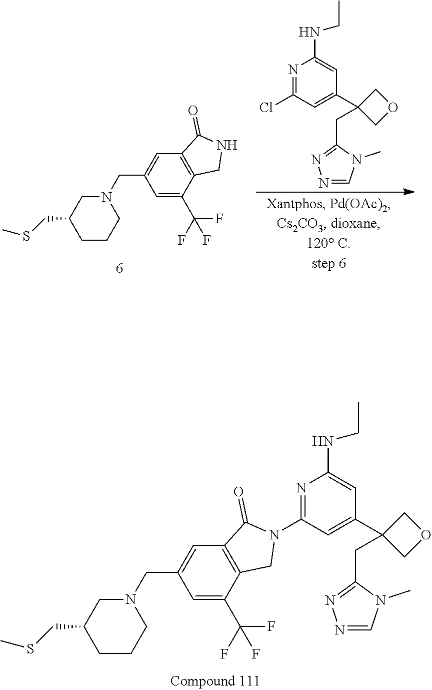 [SMILES: CSC[C@H]1CCCN(Cc2cc3c(c(C(F)(F)F)c2)CNC3=O)C1 ; CCNc1cc(C2(Cc3nncn3C)COC2)cc(Cl)n1 ; CCNc1cc(C2(Cc3nncn3C)COC2)cc(N2Cc3c(cc(CN4CCC[C@H](CSC)C4)cc3C(F)(F)F)C2=O)n1]

### Step 1: Synthesis of Compound 2

To a stirred mixture of tert-butyl (3S)-3-(hydroxymethyl)piperidine-1-carboxylate (Compound 10 g, 46.44 mmol, 1 equiv) and TEA (14 g, 138.35 mmol, 2.98 equiv) in DCM (100 mL) was added TsCl (17.5 g, 91.79 mmol, 1.98 equiv) dropwise at 0° C. under nitrogen atmosphere. The resulting mixture was stirred for overnight at room temperature. LCMS indicated the reaction was completed. The resulting mixture was concentrated under reduced pressure. The residue was purified by silica gel column chromatography, eluted with PE/EA (5:1) to afford tert-butyl (3S)-3-{[(4-methylbenzenesulfonyl)oxy]methyl}piperidine-1-carboxylate (Compound 2, 14.2 g, 74.47%) as a colorless oil. LCMS:370[M+H]⁺; ¹H NMR (300 MHz, Chloroform-d) δ 7.86-7.70 (m, 2H), 7.45-7.30 (m, 2H), 3.94-3.77 (m, 4H), 2.84-2.80 (m, 1H), 2.64 (dd, J=13.0, 9.7 Hz, 1H), 2.45 (s, 3H), 1.91-1.70 (m, 2H), 1.60-1.58 (m, 1H), 1.42 (s, 9H).

### Step 2: Synthesis of Compound 3

To a stirred solution of tert-butyl (3S)-3-{[(4-methylbenzenesulfonyl)oxy]methyl}piperidine-1-carboxylate (Compound 2, 5 g, 13.53 mmol, 1 equiv) and KI (2.25 g, 13.53 mmol, 1 equiv) in EtOH (50 mL) was added (methylsulfanyl)sodium (1.90 g, 27.06 mmol, 2 equiv) at room temperature under nitrogen atmosphere. The resulting mixture was stirred for 1 h at 80° C. under nitrogen atmosphere. LCMS indicated the reaction was completed. The resulting mixture was cooled down to room temperature, diluted with water (100 mL), extracted with EtOAc (3×100 mL). The combined organic layers were washed with brine (100 mL), dried over anhydrous Na₂SO₄. After filtration, the filtrate was concentrated under reduced pressure. This resulted in tert-butyl (3S)-3-[(methylsulfanyl)methyl]piperidine-1-carboxylate (3.1 g, 93%) as a yellow oil. LCMS (ESI, ms):246[M+H]⁺.

### Step 3: Synthesis of Compound 4

To a stirred solution of tert-butyl (3S)-3-[(methylsulfanyl)methyl]piperidine-1-carboxylate (Compound 3, 800 mg, 3.26 mmol, 1 equiv) in HCl (gas, 4N in 1,4-dioxane 30 mL) was stirred for 1 h at room temperature under nitrogen atmosphere. LCMS indicated the reaction was completed. The resulting mixture was concentrated under reduced pressure. This resulted in (3S)-3-[(methylsulfanyl)methyl]piperidine hydrochloride (Compound 4, 2.1 g, 94%) as a white solid. The crude product was used in the next step directly without further purification. LCMS (ESI, ms):146[M+H-HCl]⁺.

### Step 4: Synthesis of Compound 5

To a stirred solution of (3S)-3-[(methylsulfanyl)methyl]piperidine hydrochloride (Compound 4, 2.1 g, 11.55 mmol, 1 equiv) and methyl 2-(bromomethyl)-5-formyl-3-(trifluoromethyl)benzoate (3.01 g, 9.24 mmol, 0.8 equiv) in DCM (21 mL) was stirred for 30 min at room temperature under nitrogen atmosphere. To the above mixture was added STAB (9.80 g, 46.22 mmol, 4 equiv) at room temperature. The resulting mixture was stirred for additional 12 h at room temperature. LCMS indicated the reaction was completed. The reaction was quenched with MeOH and stirred for 30 min at room temperature. The resulting mixture was concentrated under reduced pressure. The residue was purified by silica gel column chromatography, eluted with CH₂Cl₂/MeOH (8:1) to afford methyl 2-(bromomethyl)-5-{[(3S)-3-[(methylsulfanyl)methyl]piperidin-1-yl]methyl}-3-(trifluoromethyl)benzoate (Compound 5, 1.2 g, 22%) as a yellow oil. LCMS (ESI, ms):454,456[M+H]⁺.

### Step 5: Synthesis of Compound 6

To a stirred solution methyl 2-(bromomethyl)-5-{[(3S)-3-[(methylsulfanyl)methyl]piperidin-1-yl]methyl}-3-(trifluoromethyl)benzoate (Compound 5, 600 mg, 1.32 mmol, 1 equiv) in NH₃ (g, 7N in MeOH 6 mL) was stirred for 2 h at room temperature under nitrogen atmosphere. LCMS indicated the reaction was completed. The resulting mixture was concentrated under reduced pressure. The residue was purified by reverse flash chromatography with the following conditions: column, silica gel; mobile phase, MeCN in water (0.1 FA %), 5% to 60% gradient in 30 min; detector, UV 254 nm. The collected fraction was concentrated to afford 6-{[(3S)-3-[(methylsulfanyl)methyl]piperidin-1-yl]methyl}-4-(trifluoromethyl)-2,3-dihydroisoindol-1-one (Compound 6, 200 mg, 42%) as a yellow oil. LCMS (ESI, ms):359[M+H]⁺

### Step 6: Synthesis of Compound 111

To a stirred solution of 6-{[(3S)-3-[(methylsulfanyl)methyl]piperidin-1-yl]methyl}-4-(trifluoromethyl)-2,3-dihydroisoindol-1-one (Compound 6, 120 mg, 0.33 mmol, 1 equiv) and 6-chloro-N-ethyl-4-{3-[(4-methyl-1,2,4-triazol-3-yl)methyl]oxetan-3-yl}pyridin-2-amine (103 mg, 0.33 mmol, 1 equiv) in dioxane (2.4 mL) were added Cs₂CO₃ (218 mg, 0.67 mmol, 2 equiv), RuPhos (31 mg, 0.06 mmol, 0.2 equiv) and RuPhos Palladacycle Gen. 3 (28 mg, 0.03 mmol, 0.1 equiv) at room temperature under nitrogen atmosphere. The resulting mixture was stirred for overnight at 120° C. under nitrogen atmosphere. LCMS indicated the reaction was completed. The resulting mixture was cooled down room temperature. The reaction mixture was purified by reverse flash chromatography with the following conditions: column, C18 silica gel; mobile phase, MeCN in water (0.1% FA), 5% to 60% gradient in 40 min; detector, UV 254 nm. The collected fraction was lyophilized to afford 2-[6-(ethylamino)-4-{3-[(4-methyl-1,2,4-triazol-3-yl)methyl]oxetan-3-yl}pyridin-2-yl]-6-{[(3S)-3-[(methylsulfanyl)methyl]piperidin-1-yl]methyl}-4-(trifluoromethyl)-3H-isoindol-1-one (70.5 mg, 31%) as an off-white solid. LCMS (ESI, ms):630[M+H]⁺; ¹H-NMR (300 MHz, DMSO-d₆) 8.24 (brs, 1H), 7.95-7.91 (m, 2H), 7.40 (s, 1H), 6.63 (t, J=4.8 Hz, 1H), 5.88 (s, 1H), 5.31 (s, 2H), 4.91 (d, J=6H, 2H), 4.79 (d, J=6.6 Hz, 2H), 3.72-3.70 (m, 2H), 3.68 (s, 2H), 3.22-3.18 (m, 5H), 2.85-2.82 (m, 1H), 2.72-2.69 (m, 1H), 2.40-2.38 (m, 2H), 2.02-2.00 (m, 4H), 1.82-1.80 (m, 3H), 1.78-1.55 (m, 2H), 1.12-1.10 (m, 3H), 1.05-0.98 (m, 1H).

### Synthesis of Compound 112

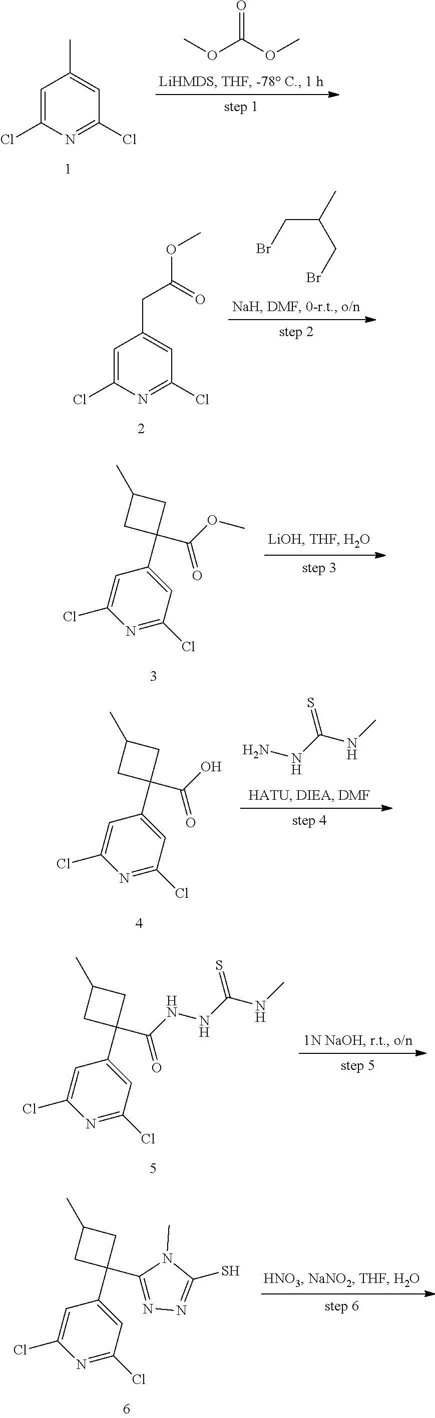

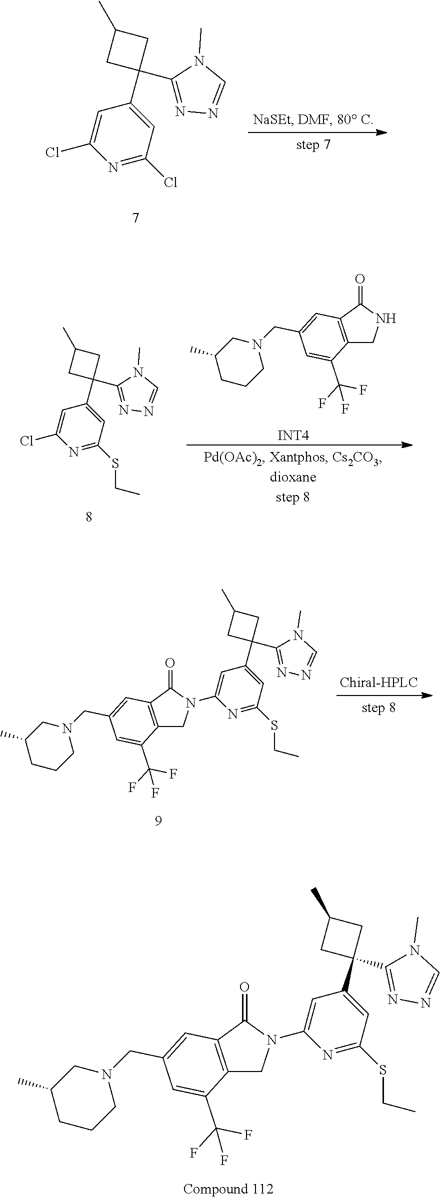

### Step 1: Synthesis of Compound 2

To a stirred mixture of 2,6-dichloro-4-methylpyridine (Compound 1, 1.0 g, 6.17 mmol, 1.0 equiv) in THE (10.0 mL) were added LiHMDS (1N in THF, 8.02 mL) at −70° C. under nitrogen atmosphere. The reaction mixture was stirred for 30 min at −70 under nitrogen atmosphere. To the above mixture was added dimethyl carbonate (1.0 g, 11.10 mmol, 1.80 equiv) at −70° C. under nitrogen atmosphere. The resulting mixture was stirred for 2 h at −70° C. under nitrogen atmosphere. ˜50% desired product could be detected by LCMS. The reaction was quenched with sat. NH₄Cl (aq., 100 mL) at 0° C. The aqueous layer was extracted with CH₂Cl₂ (3×200 mL). The resulting mixture was concentrated under vacuum. The residue was purified by silica gel column chromatography, eluted with PE/EA (3:1) to afford methyl 2-(2,6-dichloropyridin-4-yl)acetate (Compound 2, 590 mg, 41%) as an oil. LCMS (ES, m/z): 220,222 [M+1]⁺.

### Step 2: Synthesis of Compound 3

To a stirred solution of methyl 2-(2,6-dichloropyridin-4-yl)acetate (Compound 2, 5.0 g, 22.72 mmol, 1.0 equiv) in DMF (50 mL) were added NaH (2.73 g, 68.16 mmol, 3.00 equiv, 60%) in portions at 0° C. under nitrogen atmosphere. The resulting mixture was stirred for 30 min at 0° C. under nitrogen atmosphere. To the above mixture was added 1,3-dibromo-2-methylpropane (7.36 g, 34.08 mmol, 1.5 equiv) at 0° C. The resulting mixture was stirred for additional 2 h at 10° C. ˜30% desired product could be detected by LCMS. The reaction was quenched with water (200 mL) at room temperature. The mixture was acidified to pH 6 with conc. HCl. The aqueous layer was extracted with CH₂Cl₂ (3×300 mL). The resulting mixture was concentrated under vacuum. The residue was purified by reverse flash chromatography with the following conditions: column, C18 silica gel; mobile phase, ACN in water (0.1% FA), 10% to 70% gradient in 30 min; detector, UV 254 nm. The eluting isomer was extracted with CH₂Cl₂ (3×300 mL). The resulting mixture was concentrated under vacuum to afford methyl 1-(2,6-dichloropyridin-4-yl)-3-methylcyclobutane-1-carboxylate (Compound 3, 1.9 g, 15%) as a green oil. LCMS: (ES, m/s): 274,276 [M+H]⁺.

### Step 3: Synthesis of Compound 4

To a stirred solution of methyl 1-(2,6-dichloropyridin-4-yl)-3-methylcyclobutane-1-carboxylate (Compound 3, 1.8 g, 6.56 mmol, 1.0 equiv) in THE (18 mL) were added LiGH (0.31 g, 13.13 mmol, 2.0 equiv) (H₂O=9.0 mL) in portions at room temperature under nitrogen atmosphere. The resulting mixture was stirred for 1 h at room temperature under nitrogen atmosphere. LCMS indicated the reaction was completed. The reaction mixture was adjusted to pH=5 with 1N HCL. The mixture was purified by reverse flash chromatography with the following conditions: column, C18 silica gel; mobile phase, ACN in water (0.1% FA), 10% to 50% gradient in 30 min; detector, UV 254 nm. The aqueous layer was extracted with CH₂Cl₂ (3×300 mL). The resulting mixture was concentrated under vacuum to afford 1-(2,6-dichloropyridin-4-yl)-3-methylcyclobutane-1-carboxylic acid (Compound 4, 2.0 g, 83%) as a semi-solid. LCMS: (ES, m/s): 260,262 [M+H]⁺.

### Step 4: Synthesis of Compound 5

To a stirred solution of 1-(2,6-dichloropyridin-4-yl)-3-methylcyclobutane-1-carboxylic acid (Compound 4, 2.4 g, 9.22 mmol, 1.0 equiv) in DMF (24 mL) were added HATU (5.26 g, 13.84 mmol, 1.5 equiv), 1-amino-3-methylthiourea (1.16 g, 11.07 mmol, 1.2 equiv) and DIEA (2.39 g, 18.45 mmol, 2.0 equiv) in portions at 0° C. under nitrogen atmosphere. The resulting mixture was stirred for 2 h at room temperature under nitrogen atmosphere. LCMS indicated the reaction was completed. The reaction was quenched with water at room temperature. The aqueous layer was extracted with CH₂Cl₂ (3×200 mL). The resulting mixture was concentrated under vacuum. The residue was purified by silica gel column chromatography, eluted with PE/EA (2:3) to afford 1-(2,6-dichloropyridin-4-yl)-3-methyl-N-[(methylcarbamothioyl)amino]cyclobutane-1-carboxamide (2.5 g, 70%) as a white solid. LCMS: (ES, m/s): 347,349 [M+H]⁺.

### Step 5: Synthesis of Compound 6

To a stirred solution of 1-(2,6-dichloropyridin-4-yl)-3-methyl-N-[(methylcarbamothioyl)amino]cyclobutane-1-carboxamide (Compound 5, 2.3 g, 6.62 mmol, 1.0 equiv) in THE (11 mL) were added NaOH (1.0 g, 25.00 mmol, 3.77 equiv) (H₂O=11 mL) in portions at 0° C. under nitrogen atmosphere. The resulting mixture was stirred for overnight at room temperature under nitrogen atmosphere. ˜73% desired product could be detected by LCMS. The mixture was acidified to pH 5 with conc. HCl. The aqueous layer was extracted with CH₂Cl₂ (3×200 mL). The resulting mixture was concentrated under vacuum. The residue was purified by silica gel column chromatography, eluted with PE/EA (1:1) to afford 5-[1-(2,6-dichloropyridin-4-yl)-3-methylcyclobutyl]-4-methyl-1,2,4-triazole-3-thiol (Compound 6, 1.2 g, 55%) as a white solid. LCMS: (ES, m/s): 329,331 [M+H]⁺.

### Step 6: Synthesis of Compound 7

To a stirred solution of 5-[1-(2,6-dichloropyridin-4-yl)-3-methylcyclobutyl]-4-methyl-1,2,4-triazole-3-thiol (Compound 6, 1.2 g, 3.64 mmol, 1.0 equiv) in THE (12 mL) were added NaNO₂ (0.75 g, 10.93 mmol, 3.0 equiv)(H₂O=12 mL) and HNO₃ (12 mL, 1.0 mol/L) in portions at 0° C. under nitrogen atmosphere. The resulting mixture was stirred for overnight at room temperature under nitrogen atmosphere. LCMS indicated the reaction was completed. The reaction was quenched with water/ice at room temperature. The aqueous layer was extracted with CH₂Cl₂ (3×200 mL). The resulting mixture was concentrated under vacuum. The residue was purified by reverse flash chromatography with the following conditions: column, silica gel; mobile phase ACN in water (0.05% TFA), 10% to 50% gradient in 30 min; detector, UV 254 nm. The aqueous layer was extracted with CH₂Cl₂ (3×200 mL). The resulting mixture was concentrated under vacuum to afford 2,6-dichloro-4-[3-methyl-1-(4-methyl-1,2,4-triazol-3-yl)cyclobutyl]pyridine (Compound 7, 900 mg, 74%) as a green solid. LCMS: (ES, m/s): 297,299 [M+H]⁺.

### Step 7: Synthesis of Compound 8

To a stirred solution of 2,6-dichloro-4-[3-methyl-1-(4-methyl-1,2,4-triazol-3-yl)cyclobutyl]pyridine (Compound 7, 400 mg, 1.346 mmol, 1.0 equiv) and (ethylsulfanyl)sodium (147 mg, 1.75 mmol, 1.3 equiv) in THE (12 mL) at 0° C. under nitrogen atmosphere. The resulting mixture was stirred for 5 h at 80° C. under nitrogen atmosphere. LCMS indicated the reaction was completed. The resulting mixture was cooled down to room temperature and purified by reverse flash chromatography with the following conditions: column, silica gel; mobile phase ACN in water (0.1% FA), 10% to 60% gradient in 30 min; detector, UV 254 nm. The resulting mixture was concentrated under vacuum to afford 2-chloro-6-(ethylsulfanyl)-4-[3-methyl-1-(4-methyl-1,2,4-triazol-3-yl)cyclobutyl]pyridine (Compound 8, 300 mg, 65%) as a light green oil. LCMS: (ES, m/s): 323,325 [M+H]⁺.

### Step 8: Synthesis of Compound 112

To a stirred solution of 2-chloro-6-(ethylsulfanyl)-4-[3-methyl-1-(4-methyl-1,2,4-triazol-3-yl)cyclobutyl]pyridine (Compound 8, 150 mg, 0.46 mmol, 1.0 equiv) and 6-{[(3S)-3-methylpiperidin-1-yl]methyl}-4-(trifluoromethyl)-2,3-dihydroisoindol-1-one (INT4, 145 mg, 0.46 mmol, 1.0 equiv) in dioxane (8.0 mL) was added Xantphos (107 mg, 0.18 mmol, 0.4 equiv), Pd(OAc)₂ (41 mg, 0.18 mmol, 0.4 equiv) and Cs₂CO₃ (454 mg, 1.39 mmol, 3.0 equiv) in portions at room temperature under nitrogen atmosphere. The resulting mixture was stirred for 1 h at 120° C. under nitrogen atmosphere. LCMS indicated the reaction was completed. The resulting mixture was cooled down to room temperature and was purified by reverse flash chromatography with the following conditions: column, C18 silica gel; mobile phase TFA, ACN in water, 10% to 50% gradient in 10 min; detector, UV 254 nm. The resulting mixture was concentrated under vacuum to afford Compound 9. The product was separated by Chiral-HPLC with the following conditions (Column: Mobile Phase A: Hex (0.1% 2M NH₃-MeOH)-HPLC, Mobile Phase B: EtOH--HPLC; Flow rate: 20 mL/min; Gradient: 70% B to 70% B in 15.5 min; Wave Length: 220/254 nm; RT1 (min): 10.35; RT2 (min): 13.03; Sample Solvent: Hex (0.1% 2M NH₃-MeOH)-HPLC; Injection Volume: 0.5 mL; The first eluting isomer (RT=10.35 min) was concentrated to dryness and lyophilized to afford 2-[6-(ethylsulfanyl)-4-[(1r,3s)-3-methyl-1-(4-methyl-1,2,4-triazol-3-yl)cyclobutyl]pyridin-2-yl]-6-{[(3S)-3-methylpiperidin-1-yl]methyl}-4-(trifluoromethyl)-3H-isoindol-1-one (Compound 112, 16.1 mg, 5.60%) as a white solid. LCMS: (ES, m/s): 658 [M+H]⁺. ¹H-NMR (300 MHz, DMSO-d₆) 8.37 (s, 1H), 8.18 (s, 1H), 7.96 (d, J=14.8 Hz, 2H), 7.06 (s, 1H), 5.22 (s, 2H), 4.69-4.62 (m, 2H), 4.53 (t, J=7.2 Hz, 1H), 3.65 (s, 2H), 3.53 (s, 3H), 3.15-3.13 (m, 2H), 2.98-2.91 (m, 1H), 2.85-2.65 (m, 3H), 1.93 (d, J=9.6 Hz, 1H), 1.80-1.50 (m, 8H), 1.36-1.33 (m, 3H), 0.95-0.75 (m, 4H).

### Synthesis of Compound 113

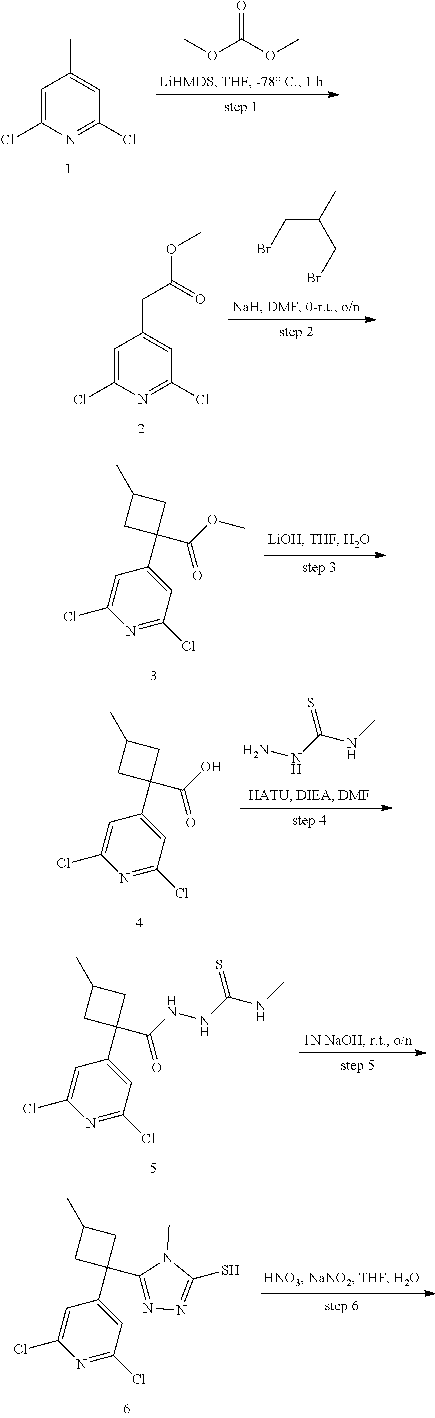

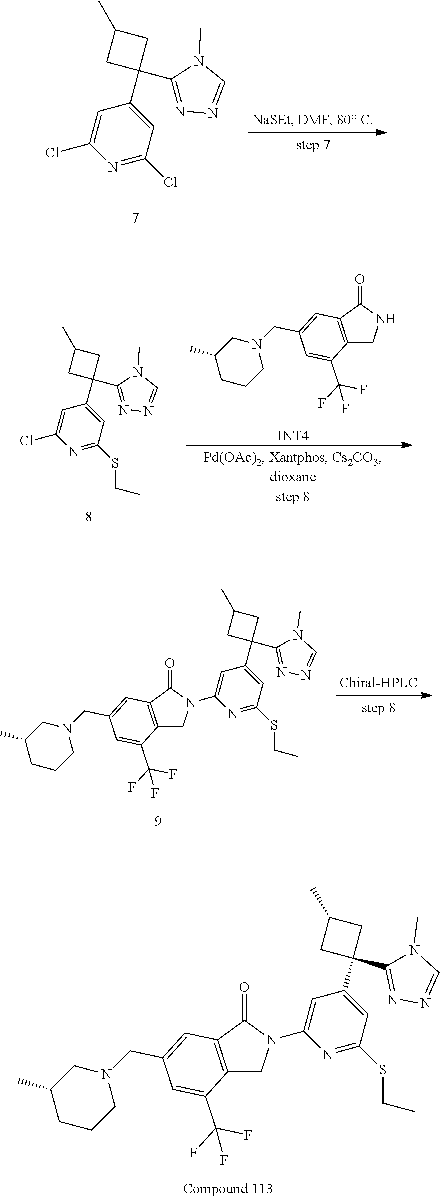

### Step 1: Synthesis of Compound 2

To a stirred mixture of 2,6-dichloro-4-methylpyridine (Compound 1, 1.0 g, 6.17 mmol, 1.0 equiv) in THE (10.0 mL) were added LiHMDS (1N in THF, 8.02 mL) at −70° C. under nitrogen atmosphere, The reaction mixture was stirred for 30 min at −70 under nitrogen atmosphere. To the above mixture was added dimethyl carbonate (1.0 g, 11.10 mmol, 1.80 equiv) at −70° C. under nitrogen atmosphere. The resulting mixture was stirred for 2 h at −70° C. under nitrogen atmosphere. ˜50% desired product could be detected by LCMS. The reaction was quenched with sat. NH₄Cl (aq., 100 mL) at 0° C. The aqueous layer was extracted with CH₂Cl₂ (3×200 mL). The resulting mixture was concentrated under vacuum. The residue was purified by silica gel column chromatography, eluted with PE/EA (3:1) to afford methyl 2-(2,6-dichloropyridin-4-yl)acetate (Compound 2, 590 mg, 41%) as an oil. LCMS (ES, m/z): 220,222 [M+1]⁺.

### Step 2: Synthesis of Compound 3

To a stirred solution of methyl 2-(2,6-dichloropyridin-4-yl)acetate (Compound 2, 5.0 g, 22.72 mmol, 1.0 equiv) in DMF (50 mL) were added NaH (2.73 g, 68.16 mmol, 3.00 equiv, 60%) in portions at 0° C. under nitrogen atmosphere. The resulting mixture was stirred for 30 min at 0° C. under nitrogen atmosphere. To the above mixture was added 1,3-dibromo-2-methylpropane (7.36 g, 34.08 mmol, 1.5 equiv) at 0° C. The resulting mixture was stirred for additional 2 h at 10° C. ˜30% desired product could be detected by LCMS. The reaction was quenched with water (200 mL) at room temperature. The mixture was acidified to pH 6 with conc. HCl. The aqueous layer was extracted with CH₂Cl₂ (3×300 mL). The resulting mixture was concentrated under vacuum. The residue was purified by reverse flash chromatography with the following conditions: column, C18 silica gel; mobile phase, ACN in water (0.1% FA), 10% to 70% gradient in 30 min; detector, UV 254 nm. The eluting isomer was extracted with CH₂Cl₂ (3×300 mL). The resulting mixture was concentrated under vacuum to afford methyl 1-(2,6-dichloropyridin-4-yl)-3-methylcyclobutane-1-carboxylate (Compound 3, 1.9 g, 15%) as a green oil. LCMS: (ES, m/s): 274,276 [M+H]⁺.

### Step 3. Synthesis of Compound 4

To a stirred solution of methyl 1-(2,6-dichloropyridin-4-yl)-3-methylcyclobutane-1-carboxylate (1.8 g, 6.56 mmol, 1.0 equiv) in THE (18 mL) were added LiGH (0.31 g, 13.13 mmol, 2.0 equiv) (H₂O=9.0 mL) in portions at room temperature under nitrogen atmosphere. The resulting mixture was stirred for 1 h at room temperature under nitrogen atmosphere. LCMS indicated the reaction was completed. The reaction mixture was adjusted to pH=5 with 1N HCL. The mixture was purified by reverse flash chromatography with the following conditions: column, C18 silica gel; mobile phase, ACN in water (0.1% FA), 10% to 50% gradient in 30 min; detector, UV 254 nm. The aqueous layer was extracted with CH₂Cl₂ (3×300 mL). The resulting mixture was concentrated under vacuum to afford 1-(2,6-dichloropyridin-4-yl)-3-methylcyclobutane-1-carboxylic acid (Compound 4, 2.0 g, 83%) as a semi-solid. LCMS: (ES, m/s): 260,262 [M+H].

### Step 4: Synthesis of Compound 5

To a stirred solution of 1-(2,6-dichloropyridin-4-yl)-3-methylcyclobutane-1-carboxylic acid (Compound 4, 2.4 g, 9.22 mmol, 1.0 equiv) in DMF (24 mL) were added HATU (5.26 g, 13.84 mmol, 1.5 equiv), 1-amino-3-methylthiourea (1.16 g, 11.07 mmol, 1.2 equiv) and DIEA (2.39 g, 18.45 mmol, 2.0 equiv) in portions at 0° C. under nitrogen atmosphere. The resulting mixture was stirred for 2 h at room temperature under nitrogen atmosphere. LCMS indicated the reaction was completed. The reaction was quenched with Water at room temperature. The aqueous layer was extracted with CH₂Cl₂ (3×200 mL). The resulting mixture was concentrated under vacuum. The residue was purified by silica gel column chromatography, eluted with PE/EA (2:3) to afford 1-(2,6-dichloropyridin-4-yl)-3-methyl-N-[(methylcarbamothioyl)amino]cyclobutane-1-carboxamide (Compound 5, 2.5 g, 70%) as a white solid. LCMS: (ES, m/s): 347,349 [M+H]⁺.

### Step 5: Synthesis of Compound 6

To a stirred solution of 1-(2,6-dichloropyridin-4-yl)-3-methyl-N-[(methylcarbamothioyl)amino]cyclobutane-1-carboxamide (Compound 5, 2.3 g, 6.62 mmol, 1.0 equiv) in THE (11 mL) were added NaOH (1.0 g, 25.00 mmol, 3.77 equiv) (H₂O=11 mL) in portions at 0° C. under nitrogen atmosphere. The resulting mixture was stirred for overnight at room temperature under nitrogen atmosphere. ˜73% desired product could be detected by LCMS. The mixture was acidified to pH 5 with conc. HCl. The aqueous layer was extracted with CH₂Cl₂ (3×200 mL). The resulting mixture was concentrated under vacuum. The residue was purified by silica gel column chromatography, eluted with PE/EA (1:1) to afford 5-[1-(2,6-dichloropyridin-4-yl)-3-methylcyclobutyl]-4-methyl-1,2,4-triazole-3-thiol (Compound 6, 1.2 g, 55%) as a white solid. LCMS: (ES, m/s): 329,331 [M+H]⁺.

### Step 6: Synthesis of Compound 7

To a stirred solution of 5-[1-(2,6-dichloropyridin-4-yl)-3-methylcyclobutyl]-4-methyl-1,2,4-triazole-3-thiol (Compound 6, 1.2 g, 3.64 mmol, 1.0 equiv) in THE (12 mL) were added NaNO₂ (0.75 g, 10.93 mmol, 3.0 equiv)(H₂O=12 mL) and HNO₃ (12 mL, 1.0 mol/L) in portions at 0° C. under nitrogen atmosphere. The resulting mixture was stirred for overnight at room temperature under nitrogen atmosphere. LCMS indicated the reaction was completed. The reaction was quenched with Water/Ice at room temperature. The aqueous layer was extracted with CH₂Cl₂ (3×200 mL). The resulting mixture was concentrated under vacuum. The residue was purified by reverse flash chromatography with the following conditions: column, silica gel; mobile phase ACN in water (0.05% TFA), 10% to 50% gradient in 30 min; detector, UV 254 nm. The aqueous layer was extracted with CH₂Cl₂ (3×200 mL). The resulting mixture was concentrated under vacuum to afford 2,6-dichloro-4-[3-methyl-1-(4-methyl-1,2,4-triazol-3-yl)cyclobutyl]pyridine (Compound 7, 900 mg, 74%) as a green solid. LCMS: (ES, m/s): 297,299 [M+H]⁺.

### Step 7: Synthesis of Compound 8

To a stirred solution of 2,6-dichloro-4-[3-methyl-1-(4-methyl-1,2,4-triazol-3-yl)cyclobutyl]pyridine (compound 7, 400 mg, 1.346 mmol, 1.0 equiv) and (ethylsulfanyl)sodium (147 mg, 1.75 mmol, 1.3 equiv) in THE (12 mL) at 0° C. under nitrogen atmosphere. The resulting mixture was stirred for 5 h at 80° C. under nitrogen atmosphere. LCMS indicated the reaction was completed. The resulting mixture was cooled down to room temperature and purified by reverse flash chromatography with the following conditions: column, silica gel; mobile phase ACN in water (0.1% FA), 10% to 60% gradient in 30 min; detector, UV 254 nm. The resulting mixture was concentrated under vacuum to afford 2-chloro-6-(ethylsulfanyl)-4-[3-methyl-1-(4-methyl-1,2,4-triazol-3-yl)cyclobutyl]pyridine (Compound 8, 300 mg, 65%) as a light green oil. LCMS: (ES, m/s): 323,325 [M+H]⁺.

### Step 8. Synthesis of Compound 113

To a stirred solution of 2-chloro-6-(ethylsulfanyl)-4-[3-methyl-1-(4-methyl-1,2,4-triazol-3-yl)cyclobutyl]pyridine (Compound 8, 150 mg, 0.46 mmol, 1.0 equiv) and 6-{[(3S)-3-methylpiperidin-1-yl]methyl}-4-(trifluoromethyl)-2,3-dihydroisoindol-1-one (INT4, 145 mg, 0.46 mmol, 1.0 equiv) in dioxane (8.0 mL) was added Xantphos (107 mg, 0.18 mmol, 0.4 equiv), Pd(OAc)₂ (41 mg, 0.18 mmol, 0.4 equiv) and Cs₂CO₃ (454 mg, 1.39 mmol, 3.0 equiv) in portions at room temperature under nitrogen atmosphere. The resulting mixture was stirred for 1 h at 120° C. under nitrogen atmosphere. LCMS indicated the reaction was completed. The resulting mixture was cooled down to room temperature and was purified by reverse flash chromatography with the following conditions: column, C18 silica gel; mobile phase TFA, ACN in water, 10% to 50% gradient in 10 min; detector, UV 254 nm. The resulting mixture was concentrated under vacuum to afford Compound 9. The product was separated by Chiral-HPLC with the following conditions (Column: Mobile Phase A: Hex (0.1% 2M NH₃-MeOH)-HPLC, Mobile Phase B: EtOH--HPLC; Flow rate: 20 mL/min; Gradient: 70% B to 70% B in 15.5 min; Wave Length: 220/254 nm; RT1 (min): 10.35; RT2 (min): 13.03; Sample Solvent: Hex (0.1% 2M NH₃-MeOH)-HPLC; Injection Volume: 0.5 mL; The second eluting isomer (RT=13.03 min) was concentrated to dryness and lyophilized to afford 2-[6-(ethylsulfanyl)-4-[(1s,3r)-3-methyl-1-(4-methyl-1,2,4-triazol-3-yl)cyclobutyl]pyridin-2-yl]-6-{[(3S)-3-methylpiperidin-1-yl]methyl}-4-(trifluoromethyl)-3H-isoindol-1-one (Compound 113, 39.4 mg, 13.85%) as a white solid. LCMS: (ES, m/s): 658 [M+H]⁺, 330 [M/2+H]⁺; ¹H-NMR (300 MHz, DMSO-d₆) 8.35 (s, 1H), 8.18 (s, 1H), 7.96-7.93 (m, 2H), 7.00 (s, 1H), 5.23 (s, 2H), 3.64 (s, 2H), 3.33 (s, 3H), 3.24 (s, 2H), 3.20-3.13 (m, 2H), 2.83-2.82 (m, 1H), 2.73-2.70 (m, 2H), 2.56-2.54 (m, 2H), 1.96-1.89 (m, 1H), 1.75-1.42 (m, 5H), 1.40-1.32 (m, 3H), 1.16-1.02 (m, 3H), 0.95-0.70 (m, 4H).

### Synthesis of Compound 114A and Compound 114B

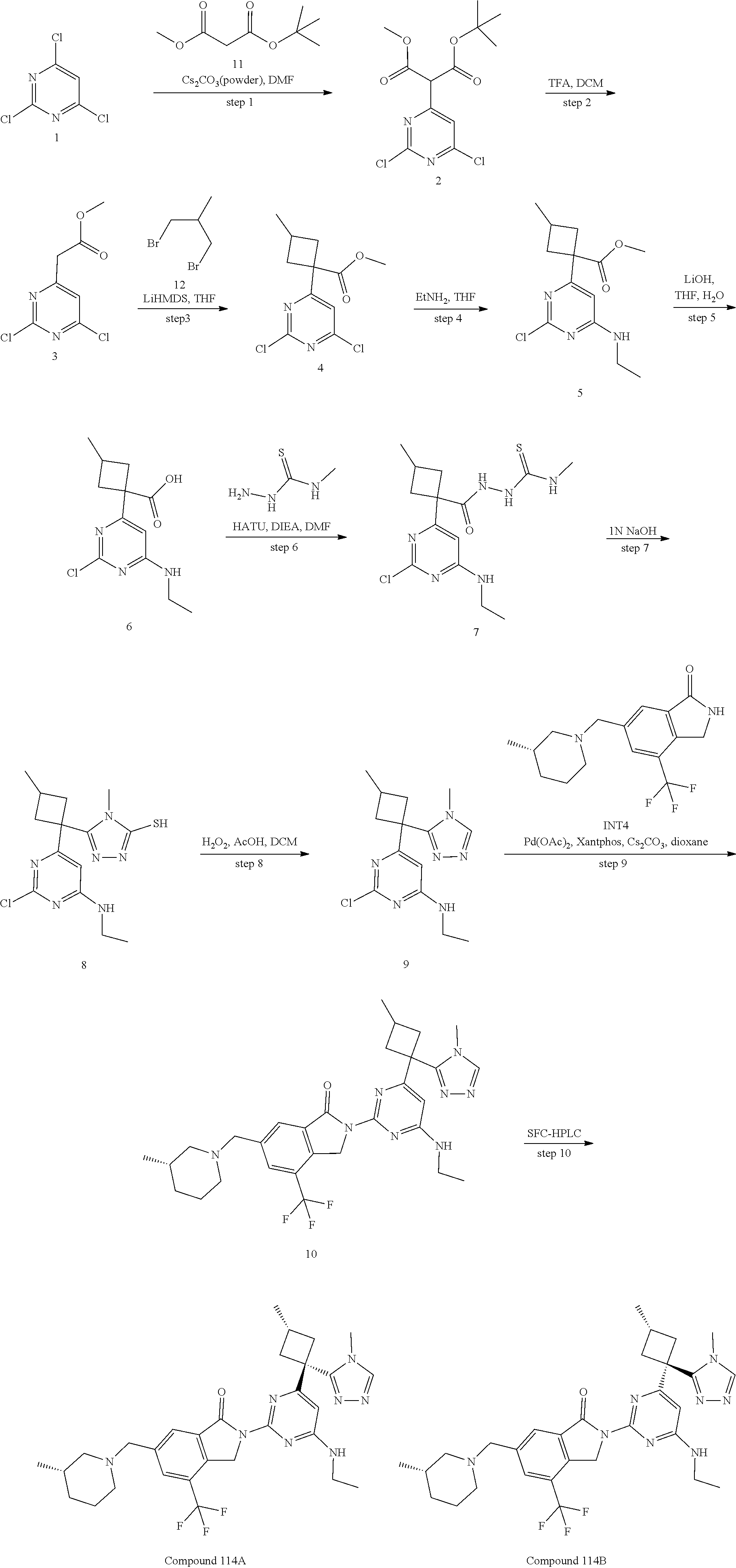

### Step 1: Synthesis of Compound 2

To a stirred mixture of 1-tert-butyl 3-methyl propanedioate (Compound 11, 35.61 g, 204.44 mmol, 1.25 equiv) and Cs₂CO₃ (106.58 g, 327.11 mmol, 2 equiv) in acetone (550 mL) was added 2,4,6-trichloropyrimidine (Compound 1, 30 g, 163.55 mmol, 1 equiv) dropwise at 10° C. The resulting mixture was stirred for 4 h at 25° C. LCMS indicated the reaction was completed. The resulting mixture was filtered, the filter cake was washed with EtOAc (3×200 mL). The filtrate was concentrated under reduced pressure. This resulted in 1-tert-butyl 3-methyl 2-(2,6-dichloropyrimidin-4-yl)propanedioate (Compound 2, 30 g, 57%) as a yellow oil. LCMS (ES, m/z): 321,323 [M+H]⁺.

### Step 2. Synthesis of Compound 3

To a stirred mixture of 1-tert-butyl 3-methyl 2-(2,6-dichloropyrimidin-4-yl)propanedioate (Compound 2, 15 g, 46.70 mmol, 1 equiv) in DCM (150 mL) was added TFA (30 mL) dropwise at 0° C. The resulting mixture was stirred for 3 h at 25° C. LCMS indicated the reaction was completed. The resulting mixture was concentrated under reduced pressure. The residue was basified to pH 8 with saturated NaHCO₃ (aq.). The resulting mixture was extracted with CH₂Cl₂ (3×100 mL). The combined organic layers were washed with brine (100 mL), dried over anhydrous Na₂SO₄. After filtration, the filtrate was concentrated under reduced pressure. The residue was purified by silica gel column chromatography, eluted with PE/EA (6:1) to afford methyl 2-(2,6-dichloropyrimidin-4-yl)acetate (Compound 3, 6 g, 51%) as a yellow oil. LCMS (ES, m/z): 221,223 [M+H]⁺.

### Step 3: Synthesis of Compound 4

To a stirred mixture of methyl 2-(2,6-dichloropyrimidin-4-yl)acetate (Compound 3, 5.6 g, 25.33 mmol, 1 equiv) in THE (80 mL) was added LiHMDS (12.72 g, 76.00 mmol, 3 equiv) at 0° C. under nitrogen atmosphere. The resulting mixture was stirred for 1 h at 0° C. under nitrogen atmosphere. To the above mixture was added methyl 1,3-dibromo-2-methylpropane (Compound 12, 10.94 g, 50.66 mmol, 2 equiv) dropwise at 0° C. The resulting mixture was stirred for additional 16 h at 25° C. LCMS indicated the reaction was completed. The reaction was quenched by the addition of water (100 mL) at room temperature. The resulting mixture was extracted with EtOAc (3×100 mL). The combined organic layers were washed with brine (100 mL), dried over anhydrous Na₂SO₄. After filtration, the filtrate was concentrated under reduced pressure. The residue was purified by silica gel column chromatography, eluted with PE/EA (6:1) to afford methyl 1-(2,6-dichloropyrimidin-4-yl)-3-methylcyclobutane-1-carboxylate (Compound 4, 2.8 g, 40%) as a yellow oil. LCMS (ES, m/z): 275,277 [M+H]⁺.

### Step 4: Synthesis of Compound 5

To a stirred mixture of methyl 1-(2,6-dichloropyrimidin-4-yl)-3-methylcyclobutane-1-carboxylate (Compound 4, 2.5 g, 9.08 mmol, 1 equiv) in THE (20 mL) was added ethylamine solution 2.0 M in THE (20 mL) dropwise at 0° C. The resulting mixture was stirred for 4 h at 25° C. LCMS indicated the reaction was completed. The resulting mixture was concentrated under reduced pressure. The residue was purified by silica gel column chromatography, eluted with PE/EA (5:1) to afford methyl 1-[2-chloro-6-(ethylamino)pyrimidin-4-yl]-3-methylcyclobutane-1-carboxylate (Compound 5, 2.3 g, 70%) as a yellow oil. LCMS (ES, m/z): 284,286 [M+H]⁺.

### Step 5: Synthesis of Compound 6

To a stirred mixture of methyl 1-[2-chloro-6-(ethylamino)pyrimidin-4-yl]-3-methylcyclobutane-1-carboxylate (Compound 5, 2.3 g, 8.10 mmol, 1 equiv) in THE (30 mL) was added LiGH (0.23 g, 9.72 mmol, 1.2 equiv) in H₂O (10 mL) dropwise at 0° C. The resulting mixture was stirred for 16 h at 25° C. LCMS indicated the reaction was completed. The resulting mixture was concentrated under reduced pressure. The mixture was acidified to pH 5 with HCl (aq.). The residue was purified by reverse flash chromatography with the following conditions: column, C18 silica gel; mobile phase, water (0.05% TFA), ACN 5% to 50% gradient in 30 min; detector, UV 254 nm. This resulted in 1-[2-chloro-6-(ethylamino)pyrimidin-4-yl]-3-methylcyclobutane-1-carboxylic acid (Compound 6, 1.4 g, 64%) as a yellow solid. LCMS (ES, m/z): 270,272 [M+H]⁺.

### Step 6: Synthesis of Compound 7

To a stirred mixture of 1-[2-chloro-6-(ethylamino)pyrimidin-4-yl]-3-methylcyclobutane-1-carboxylic acid (Compound 5, 1.2 g, 4.44 mmol, 1 equiv) and HATU (2.54 g, 6.67 mmol, 1.5 equiv) in DMF (24 mL) was added 1-amino-3-methylthiourea (0.56 g, 5.33 mmol, 1.2 equiv) and DIEA (1.73 g, 13.34 mmol, 3 equiv) at 0° C. The resulting mixture was stirred for 3 h at 25° C. LCMS indicated the reaction was completed. The reaction mixture was used in the next step directly without further purification. LCMS (ES, m/z): 357,359 [M+H].

### Step 7: Synthesis of Compound 8

To the above stirred mixture of 1-[2-chloro-6-(ethylamino)pyrimidin-4-yl]-3-methyl-N-[(methylcarbamothioyl)amino]cyclobutane-1-carboxamide (Compound 7) was added NaOH (313.82 mg, 7.84 mmol, 2 equiv) in H₂O (50 mL) at 0° C. The resulting mixture was stirred for 1 h at 50° C. LCMS indicated the reaction was completed. The mixture was allowed to cool down to room temperature. The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EA (1:1) to afford 5-{1-[2-chloro-6-(ethylamino)pyrimidin-4-yl]-3-methylcyclobutyl}-4-methyl-1,2,4-triazole-3-thiol (Compound 8, 0.6 g, 45%) as a yellow solid. LCMS (ES, m/z): 339,341 [M+H]⁺.

### Step 8: Synthesis of Compound 9

To a stirred mixture of 5-{1-[2-chloro-6-(ethylamino)pyrimidin-4-yl]-3-methylcyclobutyl}-4-methyl-1,2,4-triazole-3-thiol (Compound 8, 0.58 g, 1.71 mmol, 1 equiv) in DCM (6 mL) was added AcOH (1.5 g, 24.97 mmol, 14.59 equiv), H₂O₂ (0.5 g, 14.70 mmol, 8.59 equiv) in portions at 0° C. The resulting mixture was stirred for 1 h at room temperature. LCMS indicated the reaction was completed. The reaction mixture was basified to pH 8 with saturated NaHCO₃ (aq.). The resulting mixture was extracted with EtOAc (3×30 mL). The combined organic layers were washed with brine (30 mL), dried over anhydrous Na₂SO₄. After filtration, the filtrate was concentrated under reduced pressure. The residue was purified by silica gel column chromatography, eluted with CH₂Cl₂/MeOH (10:1) to afford 2-chloro-N-ethyl-6-[3-methyl-1-(4-methyl-1,2,4-triazol-3-yl)cyclobutyl]pyrimidin-4-amine (Compound 9, 0.25 g, 47%) as a yellow solid. LCMS (ES, m/z): 307,309 [M+H]⁺.

### Step 9: Compound 114A and Compound 114B

A mixture of 2-chloro-N-ethyl-6-[3-methyl-1-(4-methyl-1,2,4-triazol-3-yl)cyclobutyl]pyrimidin-4-amine (Compound 9, 240 mg, 0.78 mmol, 1 equiv), 6-{[(3S)-3-methylpiperidin-1-yl]methyl}-4-(trifluoromethyl)-2,3-dihydroisoindol-1-one (INT4, 256 mg, 0.82 mmol, 1.05 equiv), Pd(OAc)₂ (17 mg, 0.078 mmol, 0.1 equiv), Xantphos (90 mg, 0.15 mmol, 0.2 equiv) and Cs₂CO₃ (764 mg, 2.34 mmol, 3 equiv) in dioxane (5 mL) was stirred for 3 h at 120° C. under nitrogen atmosphere. LCMS indicated the reaction was completed. The mixture was allowed to cool down to room temperature. The resulting mixture was filtered, the filter cake was washed with EtOAc (3×20 mL). The filtrate was concentrated under reduced pressure. The residue was purified by silica gel column chromatography, eluted with CH₂Cl₂/MeOH (10:1). The crude product was re-purified by reverse flash chromatography with the following conditions: column, C18 silica gel; mobile phase, water (0.05% TFA), ACN 5% to 100% gradient in 30 min; detector, UV 254 nm. The product was separated by Prep-SFC with the following conditions: Column: CHIRALPAK IA, 3×25 cm, 5 m; Mobile Phase A: C₀₂, Mobile Phase B: ACN:MeOH=1:1 (1% 2 M NH₃-MeOH); Flow rate: 100 mL/min; Gradient: isocratic 30% B; Column Temperature: 35° C.; Back Pressure (bar): 100; Wave Length: 220 nm; RT1 (min): 3.58; RT2 (min): 4.88; The first eluting isomer (RT1=3.58 min) was concentrated and lyophilized to afford 2-[4-(ethylamino)-6-[(1s,3R)-3-methyl-1-(4-methyl-1,2,4-triazol-3-yl)cyclobutyl]pyrimidin-2-yl]-6-{[(3S)-3-methylpiperidin-1-yl]methyl}-4-(trifluoromethyl)-3H-isoindol-1-one (Compound 114A, 19.8 mg, 4%) as a white solid. LCMS (ES, m/z): 583 [M+H]⁺; ¹H-NMR (CD₃OD, 400 MHz) δ (ppm): 8.40 (s, 1H), 8.11 (s, 1H), 8.01 (s, 1H), 6.20 (brs, 1H), 5.21 (s, 2H), 4.77-4.72 (m, 2H), 4.60-4.56 (m, 1H), 3.86-3.79 (m, 5H), 3.52 (brs, 1H), 3.08-2.84 (m, 4H), 2.12 (brs, 1H), 1.86-1.56 (m, 8H), 1.35-1.20 (m, 4H), 1.05-0.85 (m, 4H).

The second eluting isomer (RT1=4.88 min) was concentrated and lyophilized to afford 2-[4-(ethylamino)-6-[(1r,3S)-3-methyl-1-(4-methyl-1,2,4-triazol-3-yl)cyclobutyl]pyrimidin-2-yl]-6-{[(3S)-3-methylpiperidin-1-yl]methyl}-4-(trifluoromethyl)-3H-isoindol-1-one (Compound 114B, 31.8 mg, 6%) as a colorless oil. LCMS (ES, m/z): 583 [M+H]⁺; ¹H-NMR (CD₃OD, 400 MHz) δ (ppm): 8.56 (s, 1H), 8.35 (s, 1H), 8.26 (s, 1H), 6.42 (s, 1H), 5.32 (s, 2H), 4.70-4.68 (m, 2H), 4.56 (s, 2H), 3.86 (s, 3H), 3.61-3.56 (m, 1H), 3.53-3.48 (m, 1H), 3.46-3.42 (m, 1H), 3.15-2.86 (m, 3H), 2.76-2.65 (m, 1H), 2.00-1.84 (m, 6H), 1.31-1.29 (m, 6H), 1.06-0.94 (m, 3H), 0.96-0.8 (m, 1H).

### Synthesis of Compound 115

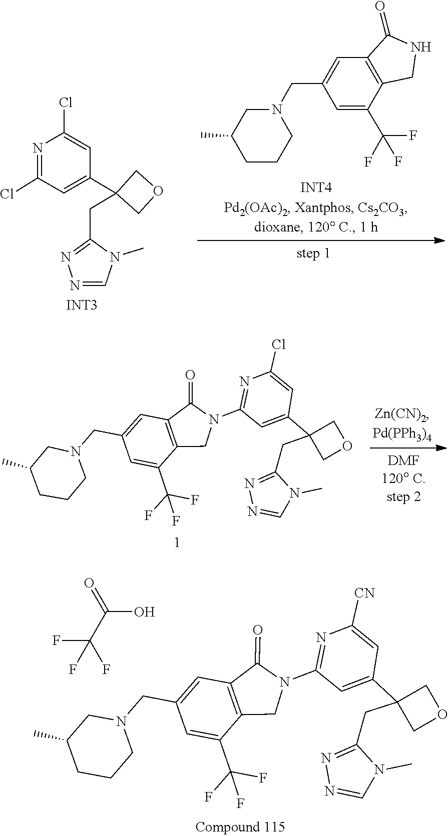

### Step 1: Synthesis of Compound 1

To a stirred mixture of 2,6-dichloro-4-{3-[(4-methyl-1,2,4-triazol-3-yl)methyl]oxetan-3-yl}pyridine (INT3, 1.05 g, 3.52 mmol, 1.10 equiv) and 6-{[(3S)-3-methylpiperidin-1-yl]methyl}-4-(trifluoromethyl)-2,3-dihydroisoindol-1-one (INT4, 1 g, 3.20 mmol, 1.00 equiv) in dioxane (10 mL) was added Cs₂CO₃ (2.09 g, 6.40 mmol, 2 equiv) and Xantphos (0.37 g, 0.64 mmol, 0.2 equiv) and dioxane (10 mL) in portions at room temperature under nitrogen atmosphere. The resulting mixture was stirred for 2 h at 120° C. under nitrogen atmosphere. The mixture was allowed to cool down to room temperature. LCMS indicated the reaction was completed. The resulting mixture was filtered, the filter cake was washed with CH₂Cl₂ (3×20 mL). The filtrate was concentrated under reduced pressure. The residue was purified by silica gel column chromatography, eluted with CH₂Cl₂/MeOH (10:1) to afford 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(trifluoromethyl)-3H-isoindol-1-one (Compound 1, 630 mg, 29%) as a green solid. LCMS (ES, m/z): 575,577 [M+H]⁺.

### Step 2: Synthesis of Compound 115

To a stirred solution of 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(trifluoromethyl)-3H-isoindol-1-one (Compound 1, 300 mg, 0.52 mmol, 1 equiv) in DMF (2 mL) was added Zn(CN)₂ (73 mg, 0.62 mmol, 1.2 equiv) and Pd(PPh₃)₄ (60 mg, 0.05 mmol, 0.1 equiv) in portions at room temperature under nitrogen atmosphere. The resulting mixture was stirred for 2 h at 120° C. under nitrogen atmosphere. The mixture was allowed to cool down to room temperature. LCMS indicated the reaction was completed. The reaction mixture was purified by reverse flash chromatography with the following conditions: column, C18 silica gel; mobile phase, ACN in water (0.05% TFA), 0% to 50% gradient in 30 min; detector, UV 254 nm. The collected fraction was concentrated under vacuum to afford 4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridine-2-carbonitrile (Compound 115, 270 mg, 74%) as an off-white solid. LCMS (ES, m/z): 566 [M+H−TFA]⁺; ¹H NMR (400 MHz, DMSO-d₆) δ9.72 (br s, 1H), 8.84 (d, J=1.2 Hz, 1H), 8.48 (s, 1H), 8.32-8.26 (m, 2H), 7.94 (s, 1H), 5.31 (s, 2H), 4.93-4.87 (m, 4H), 4.63-4.54 (m, 2H), 3.71 (s, 2H), 3.49 (s, 3H), 3.40-3.37 (m, 1H), 3.30-3.28 (m, 1H), 2.88-2.85 (m, 1H), 2.62-2.57 (m, 1H), 1.87-1.63 (m, 4H), 1.09-1.05 (m, 1H), 0.94 (d, J=15.6 Hz, 3H).

### Synthesis of Compound 116

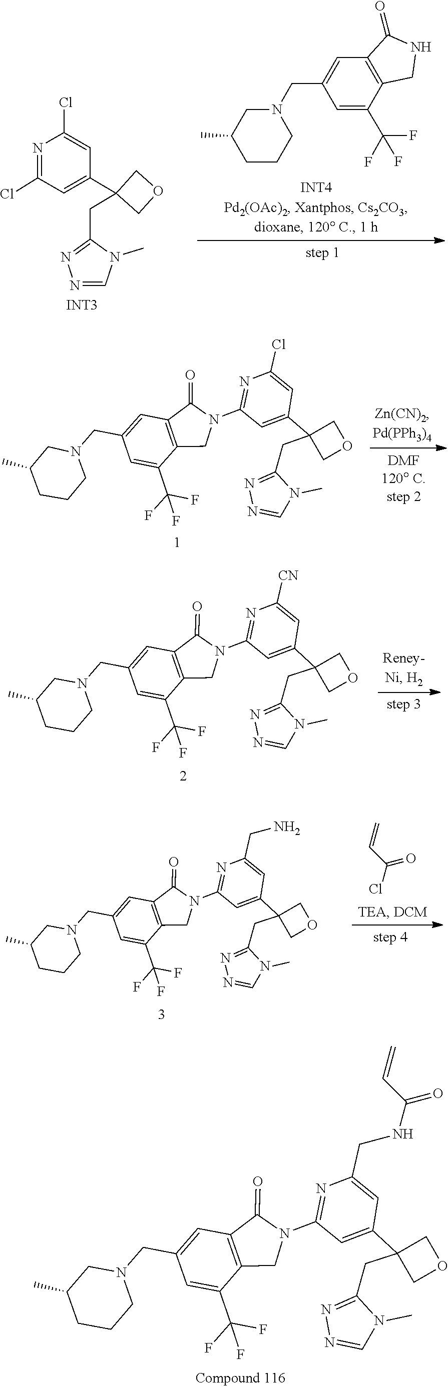

### Step 1. Synthesis of Compound 1

To a stirred mixture of 2,6-dichloro-4-{3-[(4-methyl-1,2,4-triazol-3-yl)methyl]oxetan-3-yl}pyridine (INT3, 1.05 g, 3.52 mmol, 1.10 equiv) and 6-{[(3S)-3-methylpiperidin-1-yl]methyl}-4-(trifluoromethyl)-2,3-dihydroisoindol-1-one (INT4, 1 g, 3.20 mmol, 1.00 equiv) in dioxane (10 mL) was added Cs₂CO₃ (2.09 g, 6.40 mmol, 2 equiv) and Xantphos (0.37 g, 0.64 mmol, 0.2 equiv) and dioxane (10 mL) in portions at room temperature under nitrogen atmosphere. The resulting mixture was stirred for 2 h at 120° C. under nitrogen atmosphere. The mixture was allowed to cool down to room temperature. LCMS indicated the reaction was completed. The resulting mixture was filtered, the filter cake was washed with CH₂Cl₂ (3×20 mL). The filtrate was concentrated under reduced pressure. The residue was purified by silica gel column chromatography, eluted with CH₂Cl₂/MeOH (10:1) to afford 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(trifluoromethyl)-3H-isoindol-1-one (630 mg, 29%) as a green solid. LCMS (ES, m/z): 575,577 [M+H]⁺.

### Step 2: Synthesis of Compound 2

To a stirred solution of 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(trifluoromethyl)-3H-isoindol-1-one (Compound 1, 300 mg, 0.52 mmol, 1 equiv) in DMF (2 mL) was added Zn(CN)₂ (73 mg, 0.62 mmol, 1.2 equiv) and Pd(PPh₃)₄ (60 mg, 0.05 mmol, 0.1 equiv) in portions at room temperature under nitrogen atmosphere. The resulting mixture was stirred for 2 h at 120° C. under nitrogen atmosphere. The mixture was allowed to cool down to room temperature. LCMS indicated the reaction was completed. The reaction mixture was purified by reverse flash chromatography with the following conditions: column, C18 silica gel; mobile phase, ACN in water (0.05% TFA), 0% to 50% gradient in 30 min; detector, UV 254 nm. The collected fraction was concentrated under vacuum to afford 4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridine-2-carbonitrile (Compound 2, 270 mg, 74%) as an off-white solid. LCMS (ES, m/z): 566 [M+H]⁺.

### Step 3: Synthesis of Compound 3

To a stirred solution of 4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridine-2-carbonitrile (Compound 2, 140 mg, 0.24 mmol, 1 equiv) in MeOH (4 mL) was added Reney-Ni (20 mg) in portions at room temperature. The resulting mixture was stirred for overnight at room temperature under hydrogen atmosphere. LCMS indicated the reaction was completed. The resulting mixture was filtered and the filtrate was concentrated under reduced pressure to afford 2-[6-(aminomethyl)-4-{3-[(4-methyl-1,2,4-triazol-3-yl)methyl]oxetan-3-yl}pyridin-2-yl]-6-{[(3S)-3-methylpiperidin-1-yl]methyl}-4-(trifluoromethyl)-3H-isoindol-1-one (Compound 3, 120 mg, 85%) as a yellow oil. The crude product was used in the next step directly without further purification. LCMS (ES, m/z): 570 [M+H]⁺.

### Step 4: Synthesis of Compound 116

To a stirred solution of 2-[6-(aminomethyl)-4-{3-[(4-methyl-1,2,4-triazol-3-yl)methyl]oxetan-3-yl}pyridin-2-yl]-6-{[(3S)-3-methylpiperidin-1-yl]methyl}-4-(trifluoromethyl)-3H-isoindol-1-one (Compound 3, 120 mg, 0.21 mmol, 1 equiv) in DCM (2 mL) was added acryloyl chloride (28 mg, 0.31 mmol, 1.5 equiv) and TEA (64 mg, 0.63 mmol, 3 equiv) dropwise at 0° C. The resulting mixture was stirred for 2 h at 0° C. LCMS indicated the reaction was completed. The resulting mixture was concentrated under reduced pressure. The residue was purified by Prep-HPLC with the following conditions (Column: XBridge Prep C18 OBD Column, 19*150 mm, 5 m; Mobile Phase A: Water (0.1% FA), Mobile Phase B: ACN; Flow rate: 25 mL/min; Gradient: 25% B to 53% B in 7 min, 53% B; Wave Length: 254 nm; RT1 (min): 6.30; The collected fraction was lyophilized to afford N-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)methyl]prop-2-enamide (Compound 116, 7.7 mg, 5%) as a white solid. LCMS (ES, m/z): 624 [M+H]⁺; ¹H NMR (400 MHz, DMSO-d₆) δ 8.66 (br s, 1H), 8.20 (s, 2H), 7.99-7.94 (m, 2H), 6.93 (s, 1H), 6.32-6.30 (m, 1H), 6.17-6.17 (s, 1H), 5.67-5.64 (m, 1H), 5.19 (s, 2H), 4.97 (d, J=6.0 Hz, 2H), 4.81 (m, d, J=6.0 Hz, 2H), 4.42 (d, J=6.0 Hz, 2H), 3.65 (s, 2H), 3.56 (s, 2H), 3.22 (s, 3H), 2.71-2.70 (m, 2H), 1.98-1.88 (m, 1H), 1.63-1.58 (m, 4H), 1.50-1.47 (m, 1H), 0.83-0.81 (m, 4H).

### Synthesis of Compound 117

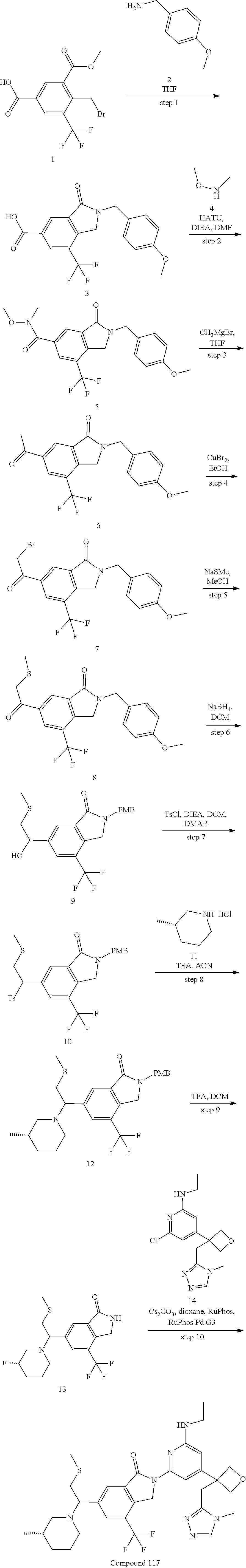

### Step 1: Synthesis of Compound 3

To a stirred mixture of 4-(bromomethyl)-3-(methoxycarbonyl)-5-(trifluoromethyl)benzoic acid (Compound 1, 6 g, 17.5 mmol, 1 equiv) in THE (300 mL) was added 4-methoxybenzenemethanamine (Compound 2, 6.00 g, 43.80 mmol, 2.49 equiv) dropwise at room temperature under nitrogen atmosphere. The resulting mixture was stirred for overnight at room temperature under nitrogen atmosphere. LCMS indicated the reaction was completed. The reaction was quenched with water. The resulting mixture was extracted with EtOAc (3×100 mL). The combined organic layers were washed with brine (100 mL), dried over anhydrous Na₂SO₄. After filtration, the filtrate was concentrated under reduced pressure. This resulted in 2-[(4-methoxyphenyl)methyl]-3-oxo-7-(trifluoromethyl)-1H-isoindole-5-carboxylic acid (Compound 3, 1.6 g, 45%) as a light yellow solid. LCMS (ESI, m/z):366 [M+H]⁺

### Step 2. Synthesis of Compound 5

To a stirred mixture of 2-[(4-methoxyphenyl)methyl]-3-oxo-7-(trifluoromethyl)-1H-isoindole-5-carboxylic acid (Compound 3, 2.5 g, 6.84 mmol, 1 equiv) and HATU (4.01 g, 10.54 mmol, 1.54 equiv) in DMF (40 mL) were added N,O-dimethylhydroxylamine hydrochloride (Compound 4, 1.00 g, 10.26 mmol, 1.50 equiv) and DIEA (2.75 g, 21.28 mmol, 3.11 equiv) at room temperature. The resulting mixture was stirred for overnight at room temperature. LCMS indicated the reaction was completed. The resulting mixture was diluted with water (5 mL). The resulting mixture was extracted with EtOAc (3×5 mL). The combined organic layers were washed with brine (5 mL), dried over anhydrous Na₂SO₄. After filtration, the filtrate was concentrated under reduced pressure. The residue was purified by silica gel column chromatography, eluted with PE/EA (5:1) to afford N-methoxy-2-[(4-methoxyphenyl)methyl]-N-methyl-3-oxo-7-(trifluoromethyl)-1H-isoindole-5-carboxamide (Compound 5, 2.5 g, 89%) as a colorless oil. LCMS:(ms, ESI): 409 [M+H]⁺

### Step 3: Synthesis of Compound 6

To a stirred mixture of N-methoxy-2-[(4-methoxyphenyl)methyl]-N-methyl-3-oxo-7-(trifluoromethyl)-1H-isoindole-5-carboxamide (Compound 5, 1 g, 2.44 mmol, 1 equiv) in THE (20 mL) was added MeMgBr (5 mL, 1 N in THF, 5 mmol) dropwise at −5° C. under nitrogen atmosphere. The resulting mixture was stirred for 1 h at room temperature under nitrogen atmosphere. LCMS indicated the reaction was completed. The reaction was quenched by the addition of sat. NH₄Cl (aq.) (20 mL) at room temperature. The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EA (5:1) to afford 6-acetyl-2-[(4-methoxyphenyl)methyl]-4-(trifluoromethyl)-3H-isoindol-1-one (Compound 6, 880 mg, 98%) as a yellow oil. LCMS:(ms, ESI):364 [M+H]⁺

### Step 4: Synthesis of Compound 7

To a stirred mixture of 6-acetyl-2-[(4-methoxyphenyl)methyl]-4-(trifluoromethyl)-3H-isoindol-1-one (Compound 6, 880 mg, 1.21 mmol, 1 equiv, 50%) in EtOH (10 mL) was added CuBr₂ (1350 mg, 6.04 mmol, 4.99 equiv) at room temperature. The resulting mixture was stirred for 1 h at 80° C. under nitrogen atmosphere. LCMS indicated the reaction was completed. The resulting mixture was diluted with water (20 mL). The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EA (3:1) to afford 6-(2-bromoacetyl)-2-[(4-methoxyphenyl)methyl]-4-(trifluoromethyl)-3H-isoindol-1-one (Compound 7, 380 mg, 70%) as a yellow solid. LCMS (ms, ESI): 442,444 [M+H]⁺

### Step 5: Synthesis of Compound 8

To a stirred solution of Sodiummethanethiolate (Compound 7, 114.23 mg, 1.631 mmol, 1.5 equiv) in MeOH (7 mL) was added 6-(2-bromoacetyl)-2-[(4-methoxyphenyl)methyl]-4-(trifluoromethyl)-3H-isoindol-1-one (350 mg, 1.08 mmol, 1 equiv) dropwise at 0° C. under nitrogen atmosphere. The resulting mixture was stirred for 10 min at 0° C. under nitrogen atmosphere. LCMS indicated the reaction was completed. The reaction was quenched with water/ice at 0° C. The precipitated solids were collected by filtration and washed with Et₂O (3×10 mL). This resulted in 2-[(4-methoxyphenyl)methyl]-6-[2-(methylsulfanyl)acetyl]-4-(trifluoromethyl)-3H-isoindol-1-one (Compound 8, 280 mg, 86%) as a yellow solid. LCMS (ESI, ms):410 [M+H]⁺

### Step 6: Synthesis of Compound 9

To a stirred solution of 2-[(4-methoxyphenyl)methyl]-6-[2-(methylsulfanyl)acetyl]-4-(trifluoromethyl)-3H-isoindol-1-one (Compound 8, 280 mg, 0.48 mmol, 1 equiv) in DCM (7 mL) was added NaBH₄ (36.96 mg, 0.97 mmol, 2 equiv) in portions at 0° C. under nitrogen atmosphere. The resulting mixture was stirred for 2 h at 0° C. under nitrogen atmosphere. LCMS indicated the reaction was completed. The reaction was quenched with water at 0° C. The resulting mixture was extracted with CH₂Cl₂ (3×10 mL). The combined organic layers were washed with brine (50 mL), dried over anhydrous Na₂SO₄. After filtration, the filtrate was concentrated under reduced pressure. This resulted in 6-[1-hydroxy-2-(methylsulfanyl)ethyl]-2-[(4-methoxyphenyl)methyl]-4-(trifluoromethyl)-3H-isoindol-1-one (Compound 9, 200 mg, 59%) as a white solid. LCMS (ESI, ms):292 [M+H]⁺

### Step 7: Synthesis of Compound 10

To a stirred mixture of 6-[1-hydroxy-2-(methylsulfanyl)ethyl]-2-[(4-methoxyphenyl)methyl]-4-(trifluoromethyl)-3H-isoindol-1-one (Compound 9, 200 mg, 0.48 mmol, 1 equiv) and DIEA (188 mg, 1.45 mmol, 3 equiv) in DCM (1 mL) was added TsCl (139 mg, 0.72 mmol, 1.5 equiv) and DMAP (6 mg, 0.05 mmol, 0.1 equiv) dropwise at 0° C. The resulting mixture was stirred for 16 h at 25° C. The reaction was quenched by the addition of water (5 mL) at room temperature. The resulting mixture was extracted with CH₂Cl₂ (3×10 mL). The combined organic layers were washed with brine (3×10 mL), dried over anhydrous Na₂SO₄. After filtration, the filtrate was concentrated under reduced pressure. The residue was purified by silica gel column chromatography, eluted with PE/EA (1:1) to afford 1-{2-[(4-methoxyphenyl)methyl]-3-oxo-7-(trifluoromethyl)-1H-isoindol-5-yl}-2-(methylsulfanyl)ethyl 4-methylbenzenesulfonate (Compound 10, 120 mg, 43%) as a yellow solid. LCMS (ES, m/z): 566 [M+H]⁺

### Step 8. Synthesis of Compound 12

To a stirred mixture of 1-{2-[(4-methoxyphenyl)methyl]-3-oxo-7-(trifluoromethyl)-1H-isoindol-5-yl}-2-(methylsulfanyl)ethyl 4-methylbenzenesulfonate (Compound 10, 120 mg, 0.21 mmol, 1 equiv) and (3S)-3-methylpiperidine hydrochloride (43 mg, 0.31 mmol, 1.5 equiv) in DCM (3 mL) was added TEA (42 mg, 0.42 mmol, 2 equiv) at 0° C. The resulting mixture was stirred for 16 h at 60° C. LCMS indicated the reaction was completed. The reaction was quenched by the addition of water (10 mL) at room temperature. The resulting mixture was extracted with CH₂Cl₂ (3×10 mL). The combined organic layers were washed with brine (3×15 mL), dried over anhydrous Na₂SO₄. After filtration, the filtrate was concentrated under reduced pressure. The residue was purified by silica gel column chromatography, eluted with CH₂Cl₂/MeOH (10:1) to afford 2-[(4-methoxyphenyl)methyl]-6-{1-[(3S)-3-methylpiperidin-1-yl]-2-(methylsulfanyl)ethyl}-4-(trifluoromethyl)-3H-isoindol-1-one (Compound 12, 80 mg, 76%) as a yellow solid. LCMS (ES, m/z): 493 [M+H]⁺

### Step 9: Synthesis of Compound 13

To a stirred mixture of 2-[(4-methoxyphenyl)methyl]-6-{1-[(3S)-3-methylpiperidin-1-yl]-2-(methylsulfanyl)ethyl}-4-(trifluoromethyl)-3H-isoindol-1-one (Compound 12, 75 mg, 0.15 mmol, 1 equiv) in DCM (100 uL) was added TFA (30 uL) at 0° C. The resulting mixture was stirred for 2 h at 25° C. LCMS indicated the reaction was completed. The resulting mixture was concentrated under reduced pressure. The residue was purified by reverse flash chromatography with the following conditions: column, C18 silica gel; mobile phase, water 0.05% TFA, ACN 5% to 50% gradient in 30 min; detector, UV 254 nm. This resulted in 6-{1-[(3S)-3-methylpiperidin-1-yl]-2-(methylsulfanyl)ethyl}-4-(trifluoromethyl)-2,3-dihydroisoindol-1-one; trifluoroacetic acid (Compound 13, 50 mg, 67%) as a yellow oil. LCMS (ES, m/z): 373 [M+H]⁺

### Step 10: Synthesis of Compound 117

A mixture of 6-{1-[(3S)-3-methylpiperidin-1-yl]-2-(methylsulfanyl)ethyl}-4-(trifluoromethyl)-2,3-dihydroisoindol-1-one (Compound 13, 40 mg, 0.10 mmol, 1 equiv), 6-chloro-N-ethyl-4-{3-[(4-methyl-1,2,4-triazol-3-yl)methyl]oxetan-3-yl}pyridin-2-amine (prepared as described in WO2020210508, 36 mg, 0.11 mmol, 1.1 equiv), Ruphos (20 mg, 0.04 mmol, 0.4 equiv), RuPhos Palladacycle Gen.3 (18 mg, 0.02 mmol, 0.2 equiv) and Cs₂CO₃ (70 mg, 0.21 mmol, 2 equiv) in dioxane (1.2 mL) was stirred for 16 h at 120° C. under nitrogen atmosphere. LCMS indicated the reaction was completed. The resulting mixture was cooled to room temperature and filtered, the filter cake was washed with EtOAc (3×10 mL). The filtrate was concentrated under reduced pressure. The residue was purified by reverse flash chromatography with the following conditions: column, C18 silica gel; mobile phase, water (0.05% TFA), ACN 5% to 50% gradient in 30 min; detector, UV 254 nm. The crude product was re-purified by Prep-HPLC with the following conditions Column: XBridge Shield RP18 OBD Column, 19×250 mm, 10 m; Mobile Phase A: Water (0.05% TFA), Mobile Phase B: ACN; Flow rate: 25 mL/min; Gradient: 32% B to 52% B in 7 min, 52% B; Wave Length: 254 nm; RT1 (min): 4.03. The collected fraction was lyophilized to 2-[6-(ethylamino)-4-{3-[(4-methyl-1,2,4-triazol-3-yl)methyl]oxetan-3-yl}pyridin-2-yl]-6-{1-[(3S)-3-methylpiperidin-1-yl]-2-(methylsulfanyl)ethyl}-4-(trifluoromethyl)-3H-isoindol-1-one; trifluoroacetic acid (Compound 117, 8.8 mg, 10%) as a colorless oil. LCMS (ES, m/z): 644 [M+H]f; ¹H-NMR (CD₃OD, 400 MHz) δ (ppm): 8.98-8.75 (m, 1H), 8.28 (s, 1H), 8.23-8.10 (m, 1H), 7.25-7.11 (m, 1H), 6.05-5.92 (m, 1H), 5.32 (s, 2H), 5.02-4.98 (m, 4H), 4.90 (s, 2H), 4.83-4.80 (m, 1H), 3.83-3.70 (m, 3H), 3.52-3.45 (m, 2H), 3.40-3.38 (m, 4H), 2.86-2.70 (m, 1H), 2.63-2.43 (m, 1H), 2.11 (s, 3H), 2.03-1.72 (m, 4H), 1.27-1.23 (m, 3H), 1.18-1.08 (m, 1H), 1.02-0.96 (m, 3H).

### Synthesis of Compound 118

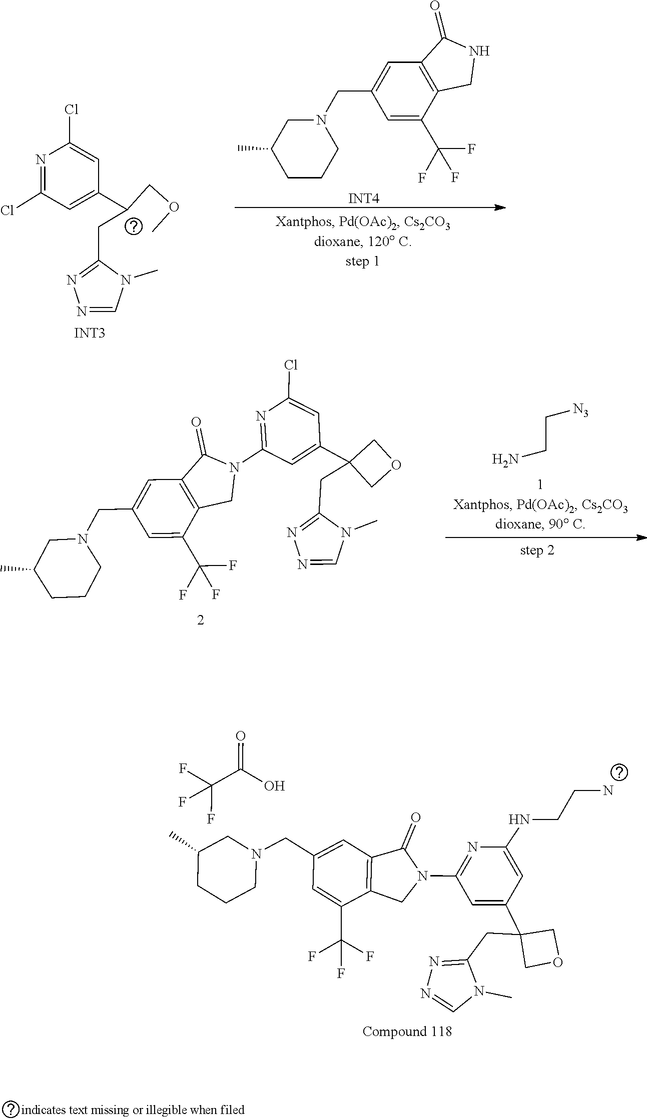

### Step 1: Synthesis of Compound 2

To a stirred mixture of 2,6-dichloro-4-{3-[(4-methyl-1,2,4-triazol-3-yl)methyl]oxetan-3-yl}pyridine (INT3, 1.05 g, 3.52 mmol, 1.10 equiv) and 6-{[(3S)-3-methylpiperidin-1-yl]methyl}-4-(trifluoromethyl)-2,3-dihydroisoindol-1-one (INT4, 1 g, 3.20 mmol, 1.00 equiv) in dioxane (10 mL) was added Cs₂CO₃ (2.09 g, 6.40 mmol, 2 equiv) and Xantphos (0.37 g, 0.64 mmol, 0.2 equiv) and Pd(OAc)₂ (112 mg, 0.50 mmol, 0.15 equiv) in portions at room temperature. The resulting mixture was stirred for 2 h at 120° C. under nitrogen atmosphere. The mixture was allowed to cool down to room temperature. LCMS indicated the reaction was completed. The resulting mixture was filtered, the filter cake was washed with CH₂Cl₂ (3×20 mL). The filtrate was concentrated under reduced pressure. The residue was purified by silica gel column chromatography, eluted with CH₂Cl₂/MeOH (10:1) to afford 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(trifluoromethyl)-3H-isoindol-1-one (Compound 2, 630 mg, 29%) as a green solid. LCMS:(ES·m/z):575,577[M+1]⁺.

### Step 2: Synthesis of Compound 118

To a stirred mixture of 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(trifluoromethyl)-3H-isoindol-1-one (Compound 2, 100 mg, 0.17 mmol, 1 equiv) and 2-azidoethanamine (Compound 1, 22 mg, 0.26 mmol, 1.5 equiv) in dioxane (1 mL) was added Cs₂CO₃ (113 mg, 0.34 mmol, 2 equiv) and Xantphos (20 mg, 0.03 mmol, 0.2 equiv) and Pd(OAc)₂ (4 mg, 0.02 mmol, 0.1 equiv) in portions at room temperature. The resulting mixture was stirred for 1.5 h at 90° C. under nitrogen atmosphere. LCMS indicated 50% product on LCMS. The resulting mixture was filtered, the filter cake was washed with DCM (3×50 mL). The filtrate was concentrated under reduced pressure. The residue was purified by silica gel column chromatography, eluted with CH₂Cl₂/MeOH (10:1) to afford 2-{6-[(2-azidoethyl)amino]-4-{3-[(4-methyl-1,2,4-triazol-3-yl)methyl]oxetan-3-yl}pyridin-2-yl}-6-{[(3S)-3-methylpiperidin-1-yl]methyl}-4-(trifluoromethyl)-3H-isoindol-1-one (30 mg, crude). The crude product (30 mg) was purified by Prep-HPLC with the following conditions (Column: XSelect CSH Prep C18 OBD Column, 19*250 mm, 5 m; Mobile Phase A: Water (0.05% TFA), Mobile Phase B: ACN; Flow rate: 25 mL/min; Gradient: 20% B to 35% B in 10 min, 35% B; Wave Length: 254 nm; RT1 (min): 8.77; The collected fraction was lyophilize to afford 2-{6-[(2-azidoethyl)amino]-4-{3-[(4-methyl-1,2,4-triazol-3-yl)methyl]oxetan-3-yl}pyridin-2-yl}-6-{[(3S)-3-methylpiperidin-1-yl]methyl}-4-(trifluoromethyl)-3H-isoindol-1-one (Compound 118, 8.5 mg, 8%) as a yellow solid. LCMS:(ES·m/z):625[M+1]⁺; ¹H NMR (400 MHz, DMSO-d₆) δ 9.81 (s, 1H), 8.45 (s, 1H), 8.27-8.19 (m, 2H), 7.44 (s, 1H), 6.98 (br s, 1H), 6.07 (s, 1H), 5.24 (s, 2H), 4.91-4.85 (m, 2H), 4.79-4.67 (m, 2H), 4.52 (s, 2H), 3.55 (s, 1H), 3.49-3.48 (m, 4H), 3.39-3.36 (m, 1H), 3.29-3.21 (m, 4H), 2.86-2.84 (m, 1H), 2.63-2.56 (m, 1H), 2.49-2.48 (m, 1H), 1.85-1.62 (m, 4H), 1.10-1.12 (m, 1H), 0.94 (d, J=5.6 Hz, 3H)

### Synthesis of Compound 119

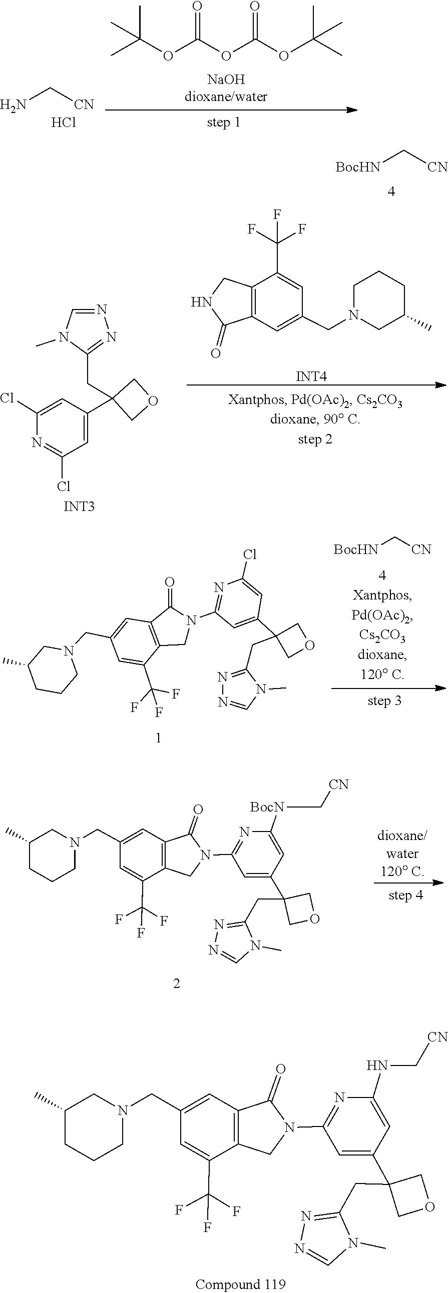

### Step 1: Synthesis of Compound 4

To a stirred solution of aminoacetonitrile hydrochloride (500 mg, 5.4 mmol, 1 equiv) in dioxane (10 mL) was added 1M NaOH aqueous solution (217 mg, 5.4 mmol, 1 equiv). Reaction was cooled to 0° C. and di-tert-butyl dicarbonate (1.18 gr, 5.4 mmol, 1 equiv) dissolved in 5 mL of dioxane was added dropwise. Resulting mixture was vigorously stirred O.N. In the morning TLC control indicated complete consumption of starting material. Reaction was quenched with aqueous solution of saturated NaHCO₃, and extracted with dichloromethane (3×100 mL). The combined organic layers were washed with brine (3×100 mL), dried over Na₂SO₄, and concentrated under vacuo to afford tert-butyl (cyanomethyl)carbamate (Compound 4, 800 mg, 95%) as a colorless oil. ¹H NMR (400 MHz, CDCl₃) δ 5.03 (s, 1H), 4.06 (m, 2H), 1.44 (s, 9H).

### Step 2: Synthesis of Compound 1

To a stirred mixture of 2,6-dichloro-4-(3-((4-methyl-4H-1,2,4-triazol-3-yl)methyl)oxetan-3-yl)pyridine (INT3, 235 mg, 0.786 mmol, 1 equiv) and (S)-6-((3-methylpiperidin-1-yl)methyl)-4-(trifluoromethyl)isoindolin-1-one (INT4, 245 mg, 0.786 mmol, 1 equiv) in dioxane (7 mL) was added Cs₂CO₃ (512 mg, 1.57 mmol, 2 equiv) and Xantphos (90.8 mg, 0.157 mmol, 0.2 equiv) and Pd(OAc)₂ (17.6 mg, 0.0786 mmol, 0.1 equiv) in portions at room temperature. The resulting mixture was stirred for 1 h at 90° C. under nitrogen atmosphere. The mixture was allowed to cool down to room temperature. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (9:1) to afford (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 1, 300 mg, 66.4%) as a green solid. LCMS:(ES·m/z):575,577[M+1]⁺.

### Step 3: Synthesis of Compound 2

To a stirred mixture of (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 1, 40 mg, 0.07 mmol, 1 equiv) and tert-butyl (cyanomethyl)carbamate (Compound 4, 110 mg, 0.7 mmol, 10 equiv) in dioxane (1.2 mL) was added Cs₂CO₃ (45 mg, 0.14 mmol, 2 equiv) and Xantphos (8.1 mg, 0.014 mmol, 0.2 equiv) and Pd(OAc)₂ (1.6 mg, 0.007 mmol, 0.1 equiv) in portions at room temperature. The resulting mixture was stirred for 40 min at 120° C. under nitrogen atmosphere. LCMS indicated the reaction was completed. The resulting mixture was filtered, filtrate was concentrated under reduced pressure. The residue was purified by silica gel column chromatography, eluted with CH₂Cl₂/MeOH (10:1) to afford tert-butyl (S)-(cyanomethyl) (4-(3-((4-methyl-4H-1,2,4-triazol-3-yl)methyl)oxetan-3-yl)-6-(6-((3-methylpiperidin-1-yl)methyl)-1-oxo-4-(trifluoromethyl)isoindolin-2-yl)pyridin-2-yl)carbamate (Compound 2, 25 mg, 52%) as a white solid. LCMS:(ES·m/z):695[M+1]⁺.

### Step 4: Synthesis of Compound 119

Tert-butyl(S)-(cyanomethyl) (4-(3-((4-methyl-4H-1,2,4-triazol-3-yl)methyl)oxetan-3-yl)-6-(6-((3-methylpiperidin-1-yl)methyl)-1-oxo-4-(trifluoromethyl)isoindolin-2-yl)pyridin-2-yl)carbamate (Compound 2, 25 mg, 0.042 mmol) was dissolved in 1/1 mixture of dioxane/water (1 ml) and heated in microwave initiator at 120C for 15 min. Product was extracted with dichloromethane and purified on silica gel column chromatography, eluted with CH₂Cl₂/MeOH (3:1) to afford (S)-2-((4-(3-((4-methyl-4H-1,2,4-triazol-3-yl)methyl)oxetan-3-yl)-6-(6-((3-methylpiperidin-1-yl)methyl)-1-oxo-4-(trifluoromethyl)isoindolin-2-yl)pyridin-2-yl)amino)acetonitrile (7 mg, 30%) as a white solid. LCMS:(ES·m/z):595[M+1]⁺; ¹H NMR (300 MHz, DMSO-d₆) δ 8.25 (s, 1H), 7.95 (d, J=17.2 Hz, 2H), 7.61 (d, J=1.2 Hz, 1H), 7.29 (t, J=5.8 Hz, 1H), 6.18 (d, J=1.2 Hz, 1H), 5.27-5.22 (m, 2H), 4.92 (d, J=6.1 Hz, 2H), 4.80 (d, J=6.1 Hz, 2H), 4.29 (d, J=5.7 Hz, 2H), 3.66 (s, 2H), 3.52 (s, 2H), 3.26 (s, 3H), 2.72 (d, J=8.7 Hz, 2H), 2.01-1.88 (m, 1H), 1.66 (d, J=13.1 Hz, 3H), 1.59 (s, 1H), 1.48 (d, J=11.8 Hz, 1H), 1.23 (s, 4H), 0.89 (m, 4H).

### Synthesis of Compound 120

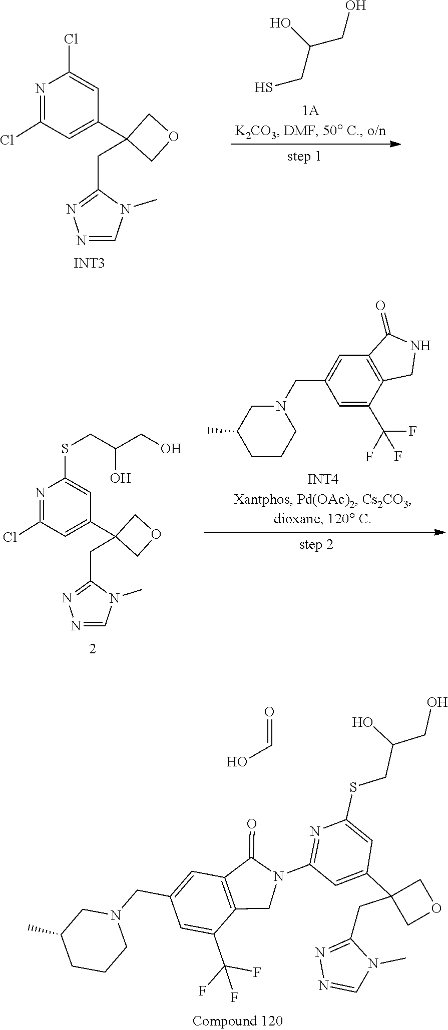

### Step 1: Synthesis of Compound 2

To a stirred solution of 2,6-dichloro-4-{3-[(4-methyl-1,2,4-triazol-3-yl)methyl]oxetan-3-yl}pyridine (INT3, 200 mg, 0.66 mmol, 1 equiv) and 3-mercaptopropane-1,2-diol (Compound 1A, 86 mg, 0.80 mmol, 1.2 equiv) in DMF (2 mL) was added K₂CO₃ (184 mg, 1.33 mmol, 2 equiv) at room temperature under nitrogen atmosphere. The resulting mixture was stirred for overnight at 50° C. under nitrogen atmosphere. LCMS indicated the reaction was completed. The mixture was allowed to cool down to room temperature. The reaction mixture was purified by reverse flash chromatography with the following conditions: column, C18 silica gel; mobile phase, ACN in water (0.1% FA), 5% to 50% gradient in 30 min; detector, UV 254 nm. The collected fraction was concentrated to afford 3-[(6-chloro-4-{3-[(4-methyl-1,2,4-triazol-3-yl)methyl]oxetan-3-yl}pyridin-2-yl)sulfanyl]propane-1,2-diol (Compound 2, 190 mg, 76%) as a white solid. LCMS (ESI, ms): 370,372[M+H]⁺

### Step 2: Synthesis of Compound 120

To a stirred solution of 3-[(6-chloro-4-{3-[(4-methyl-1,2,4-triazol-3-yl)methyl]oxetan-3-yl}pyridin-2-yl)sulfanyl]propane-1,2-diol (Compound 2, 100 mg, 0.27 mmol, 1 equiv) and 6-{[(3S)-3-methylpiperidin-1-yl]methyl}-4-(trifluoromethyl)-2,3-dihydroisoindol-1-one (INT4, 92 mg, 0.29 mmol, 1.1 equiv) in 1,4-dioxane (2 mL) was added Cs₂CO₃ (263 mg, 0.810 mmol, 3 equiv), XantPhos (31 mg, 0.05 mmol, 0.2 equiv) and Pd(OAc)₂ (6 mg, 0.03 mmol, 0.1 equiv) at room temperature under nitrogen atmosphere. The resulting mixture was stirred for 1 h at 120° C. under nitrogen atmosphere. LCMS indicated the reaction was completed. The mixture was allowed to cool down to room temperature. The mixture was purified by reverse flash chromatography with the following conditions: column, C18 silica gel; mobile phase, ACN in water (0.1% FA), 5% to 50% gradient in 30 min; detector, UV 254 nm. The collected fraction was lyophilized to afford 2-{6-[(2,3-dihydroxypropyl)sulfanyl]-4-{3-[(4-methyl-1,2,4-triazol-3-yl)methyl]oxetan-3-yl}pyridin-2-yl}-6-{[(3S)-3-methylpiperidin-1-yl]methyl}-4-(trifluoromethyl)-3H-isoindol-1-one; formic acid (33.8 mg, 17%) as a white solid. LCMS (ESI, ms):646[M+H−FA]⁺; ¹H NMR (300 MHz, DMSO-d₆) δ 8.25 (s, 1H), 8.14 (s, 1H), 8.03 (s, 1H), 7.99 (s, 1H), 7.94 (s, 1H), 6.96 (s, 1H), 5.24 (s, 2H), 4.92 (d, J=6.3 Hz, 2H), 4.84 (d, J=6.3 Hz, 2H), 3.67-3.64 (m, 4H), 3.57 (s, 3H), 3.48-3.41 (m, 4H), 3.07-2.97 (m, 1H), 2.76-2.70 (m, 2H), 1.96-1.93 (m, 1H), 1.72-1.50 (m, 5H), 0.88-0.75 (m, 4H).

### Synthesis of Compound 121

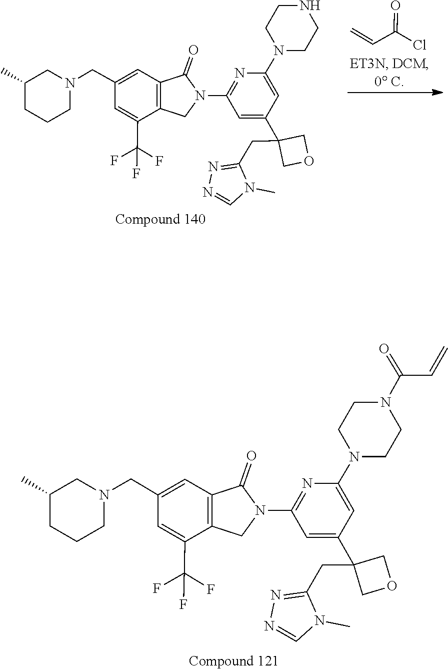

To a stirred mixture of (S)-2-(4-(3-((4-methyl-4H-1,2,4-triazol-3-yl)methyl)oxetan-3-yl)-6-(piperazin-1-yl)pyridin-2-yl)-6-((3-methylpiperidin-1-yl)methyl)-4-(trifluoromethyl)isoindolin-1-one (Compound 140, 35 mg, 0.056 mmol, 1 equiv) and triethylamine (7.4 mg, 0.073 mmol, 1.3 equiv) in dichloromethane (1.5 mL) previously cooled at 0° C. was added acryloyl chloride (6.6 mg, 0.073 mmol, 1.3 equiv) in dichloromethane (1.5 mL). The resulting mixture was allowed to reach room temperature and stirred for 2 hours. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (3:1) to afford (S)-2-(6-(4-acryloylpiperazin-1-yl)-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 121, 15 mg, 39%) as a white solid. LCMS:(ES·m/z):679[M+1]⁺; ¹H NMR (400 MHz, DMSO-d₆) δ 10.72 (s, 1H), 8.34 (s, 1H), 8.30 (s, 1H), 8.23 (s, 1H), 7.56 (d, J=1.0 Hz, 1H), 6.88 (dd, J=16.7, 10.4 Hz, 1H), 6.33 (s, 1H), 6.15 (dd, J=16.7, 2.4 Hz, 1H), 5.71 (dd, J=10.4, 2.4 Hz, 1H), 5.22 (s, 2H), 4.92 (d, J=6.2 Hz, 2H), 4.84 (d, J=6.2 Hz, 2H), 4.49 (s, 1H), 3.69 (s, 2H), 3.64 (s, 2H), 3.56 (s, 2H), 3.53 (s, 4H), 3.18 (d, J=15.2 Hz, 4H), 3.06 (qd, J=7.3, 4.8 Hz, 3H), 2.79 (s, 1H), 2.55 (s, 1H), 1.96 (s, 1H), 1.79 (s, 1H), 1.73 (d, J=13.7 Hz, 2H), 1.25-1.14 (m, 6H), 0.85 (t, J=5.0 Hz, 4H).

### Synthesis of Compound 122

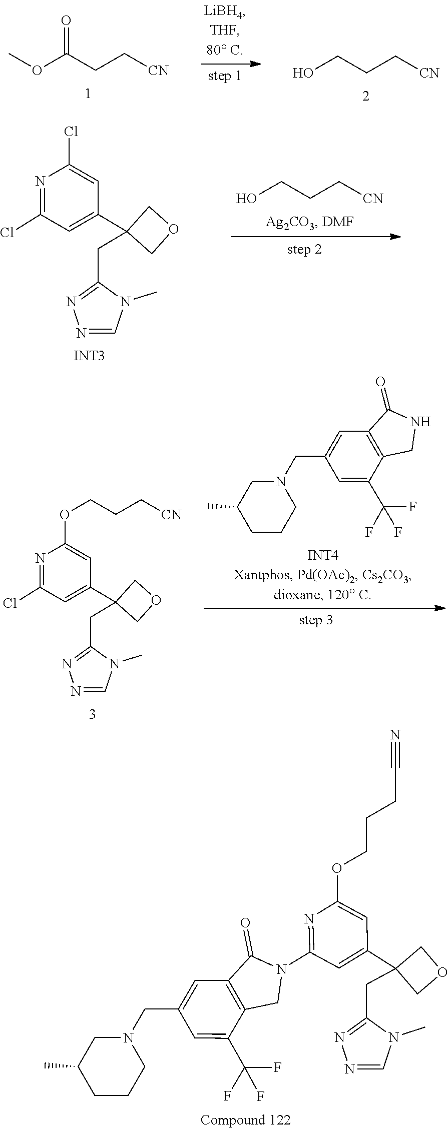

### Step 1: Synthesis of Compound 2

To a stirred solution of methyl 3-cyanopropanoate (Compound 1, 5.0 g, 44.20 mmol, 1.0 equiv) in THE (50 mL) at room temperature under nitrogen atmosphere. To the above mixture was added LiBH₄ (0.58 g, 26 mmol, 0.6 equiv) in portions at room temperature. The resulting mixture was stirred for overnight at 80° C. under nitrogen atmosphere. TLC indicated the reaction was completed. The reaction was quenched with water/ice at room temperature. The aqueous layer was extracted with EA (3×300 mL). The resulting mixture was concentrated under vacuum. The residue was purified by silica gel column chromatography, eluted with PE/EA (1:1) to afford 4-hydroxybutanenitrile (700 mg, 18%) as a light color oil. ¹H-NMR (300 MHz, DMSO-d₆) 4.68 (t, J=6 Hz, 1H), 3.49-3.42 (m, 2H), 2.53-2.48 (m, 2H), 1.74-1.65 (m, 2H).

### Step 2: Synthesis of Compound 3

To a stirred solution of 4-hydroxybutanenitrile (Compound 2, 63 mg, 0.73 mmol, 1.10 equiv) in DMF (4.0 mL) was added NaH (54 mg, 1.33 mmol, 2.00 equiv, 60%) at 0° C. under nitrogen atmosphere. To the above mixture was added 2,6-dichloro-4-{3-[(4-methyl-1,2,4-triazol-3-yl)methyl]oxetan-3-yl}pyridine (INT3, 200 mg, 0.66 mmol, 1.00 equiv) in portions over 30 min at 0° C. The resulting mixture was stirred for additional 2 h at room temperature. LCMS indicated the reaction was completed. The residue was purified by reverse flash chromatography with the following conditions: column, silica gel; mobile phase FA, MeCN in water, 10% to 50% gradient in 40 min; detector, UV 254 nm. The resulting mixture was concentrated under vacuum to afford 4-[(6-chloro-4-{3-[(4-methyl-1,2,4-triazol-3-yl)methyl]oxetan-3-yl}pyridin-2-yl)oxy]butanenitrile (Compound 3, 180 mg, 69%) as a semi-solid. LCMS: (ES, m/s): 348,350 [M+H]⁺.

### Step 3: Synthesis of Compound 122

To a stirred solution of 4-[(6-chloro-4-{3-[(4-methyl-1,2,4-triazol-3-yl)methyl]oxetan-3-yl}pyridin-2-yl)oxy]butanenitrile (120 mg, 0.34 mmol, 1.0 equiv) and 6-{[(3S)-3-methylpiperidin-1-yl]methyl}-4-(trifluoromethyl)-2,3-dihydroisoindol-1-one (107 mg, 0.35 mmol, 1.0 equiv) in dioxane (5.0 mL) was added Xantphos (79 mg, 0.13 mmol, 0.4 equiv), Pd(OAc)₂ (31 mg, 0.13 mmol, 0.4 equiv) and Cs₂CO₃ (337 mg, 1.03 mmol, 3.0 equiv) in portions at room temperature under nitrogen atmosphere. The resulting mixture was stirred for 1 h at 120° C. under nitrogen atmosphere. LCMS indicated the reaction was completed. The reaction mixture was cooled down to room temperature and purified by reverse flash chromatography with the following conditions: column, C18 silica gel; mobile phase TFA (0.05%), ACN in water, 10% to 50% gradient in 30 min; detector, UV 254 nm. The resulting mixture was concentrated under vacuum to afford crude product. The crude product was re-purified by Prep-HPLC with the following conditions (Column: Mobile Phase A: Water (0.05% TFA), Mobile Phase B: ACN; Flow rate: 25 mL/min; Gradient: 25% B to 45% B in 7 min, 45%). The collected fraction was lyophilized to afford 4-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)oxy]butanenitrile; trifluoroacetic acid (Compound 122, 53 mg, 20%) as a white solid. LCMS: (ES, m/s): 624 [M+H]⁺, 646 [M+Na]⁺; ¹H-NMR (300 MHz, DMSO-d₆) 9.94 (br s, 1H), 8.58 (s, 1H), 8.26-8.22 (m, 2H), 7.97 (s, 1H), 6.54 (s, 1H), 5.29 (s, 2H), 4.92 (d, J=6 Hz, 2H), 4.83 (d, J=6 Hz, 2H), 4.80-4.75 (m, 2H), 4.54 (s, 2H), 4.40-4.36 (m, 2H), 3.65 (s, 2H), 3.40 (s, 3H), 3.28-3.27 (m, 1H), 2.90-2.85 (m, 1H), 2.67-2.62 (m, 2H), 2.12-2.03 (m, 2H), 1.87-1.59 (m, 4H), 1.13-1.02 (m, 1H), 0.88 (d, J=6 Hz, 3H).

### Synthesis of Compound 123

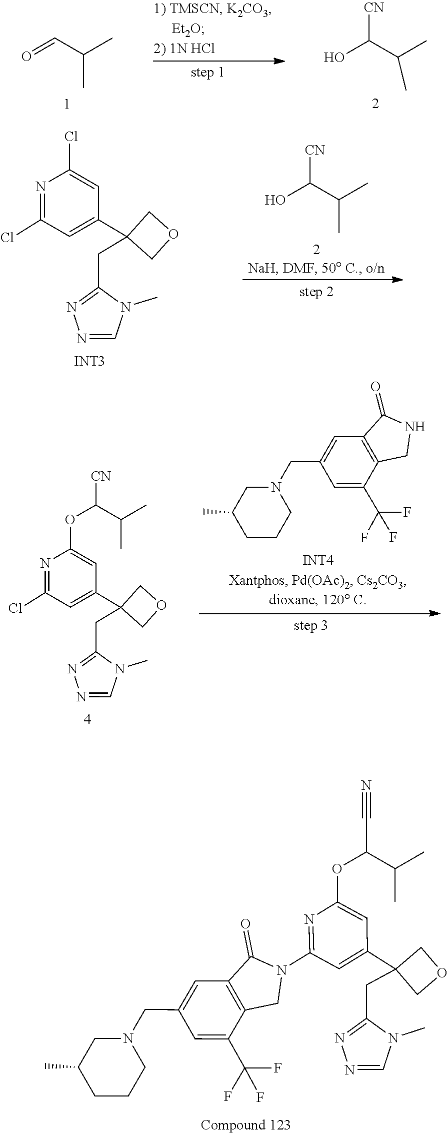

### Step 1: Synthesis of Compound 2

To a stirred solution of isobutyraldehyde (Compound 1, 5 g, 69.34 mmol, 1 equiv) in Et₂O (10 mL) was added K₂CO₃ (1.92 g, 13.86 mmol, 0.2 equiv) in portions at 0° C. under air atmosphere. Then was added TMSCN (13.76 g, 138.68 mmol, 2 equiv) dropwise at 0° C. The resulting mixture was stirred for 6 h at room temperature under air atmosphere. Desired product could be detected by GCMS. The reaction was quenched by the addition of NaHCO₃ (20 mL) at 0° C. The aqueous layer was extracted with Et₂O (3×40 mL). The combined organic layer was concentrated under vacuum. To the above mixture was added HCl (1N, 30 ml) dropwise over 10 min at 0° C. The resulting mixture was stirred for additional 2 h at room temperature. GCMS indicated the reaction was completed. The aqueous layer was extracted with Et₂O (3×40 mL). The organic layer was washed with of NaHCO₃ (aq, 2×20 mL). The combined organic layer was concentrated under vacuum. The residue was purified by silica gel column chromatography, eluted with CH₂Cl₂/MeOH (10:1) to afford 2-hydroxy-3-methylbutanenitrile (Compound 2, 5 g, 72%) as a white oil. GCMS:(ES·m/z):99[M].

### Step 2: Synthesis of Compound 4

To a stirred solution 2-hydroxy-3-methylbutanenitrile (Compound 2, 132 mg, 1.33 mmol, 2 equiv) in DMF (2 mL) was added NaH (53 mg, 1.33 mmol, 60%, 2 equiv) in portions at 0° C. under air atmosphere. The resulting mixture was stirred for 1 h at 0° C. under air atmosphere. To the above mixture was added 2,6-dichloro-4-{3-[(4-methyl-1,2,4-triazol-3-yl)methyl]oxetan-3-yl}pyridine (INT3, 200 mg, 0.66 mmol, 1 equiv) in portions at room temperature. The resulting mixture was stirred for additional overnight at 50° C. LCMS indicated the reaction 50% product and 14% the starting material. The reaction mixture was cooled down to room temperature reaction and quenched with water/ice at room temperature. The aqueous layer was extracted with EA (3×300 mL). The resulting mixture was concentrated under vacuum. The residue was purified by reverse flash chromatography with the following conditions: column, C18 silica gel; mobile phase, ACN in water (0.05% TFA), 0% to 50% gradient in 40 min; detector, UV 254 nm. This resulted in 2-[(6-chloro-4-{3-[(4-methyl-1,2,4-triazol-3-yl)methyl]oxetan-3-yl}pyridin-2-yl)oxy]-3-methylbutanenitrile (Compound 4, 160 mg, 66%) as a yellow solid. LCMS:(ES·m/z): 362,364[M+1]⁺.

### Step 3: Synthesis of Compound 123

To a stirred mixture of 2-[(6-chloro-4-{3-[(4-methyl-1,2,4-triazol-3-yl)methyl]oxetan-3-yl}pyridin-2-yl)oxy]-3-methylbutanenitrile (Compound 4, 150 mg, 0.41 mmol, 1.00 equiv) and 6-{[(3S)-3-methylpiperidin-1-yl]methyl}-4-(trifluoromethyl)-2,3-dihydroisoindol-1-one (INT4, 129 mg, 0.41 mmol, 1.00 equiv) in dioxane (2 mL) was added Cs₂CO₃ (270 mg, 0.83 mmol, 2 equiv) and Xantphos (47 mg, 0.08 mmol, 0.2 equiv) and Pd(OAc)₂ (9.31 mg, 0.04 mmol, 0.1 equiv) in portions at room temperature under nitrogen atmosphere. The resulting mixture was stirred for 2 h at 120° C. under nitrogen atmosphere. LCMS indicated the reaction was 44% product. The reaction mixture was cooled down to room temperature and purified by reverse flash chromatography with the following conditions: column, C18 silica gel; mobile phase FA (0.1%), ACN in water, 5% to 60% gradient in 40 min; detector, UV 254 nm. The collected fraction was lyophilized to afford 3-methyl-2-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)oxy]butanenitrile; formic acid (31.3 mg, 10%) as a white solid. LCMS:(ES·m/z):638[M+1]⁺; H NMR (400 MHz, DMSO-d₆) δ8.26 (s, 1H), 8.15 (s, 1H), 8.08 (s, 1H), 8.01 (s, 1H), 7.96 (s, 1H), 6.68 (s, 1H), 5.59 (d, J=8 Hz, 1H), 5.33-5.24 (m, 2H), 4.92-4.84 (m, 4H), 3.67-3.59 (m, 4H), 3.36 (s, 3H), 2.75-2.71 (m, 2H), 2.41-2.32 (m, 1H), 1.97-1.94 (m, 1H), 1.68-1.59 (m, 5H), 1.14-1.11 (m, 6H), 0.83-0.82 (m, 4H).

### Synthesis of Compound 124

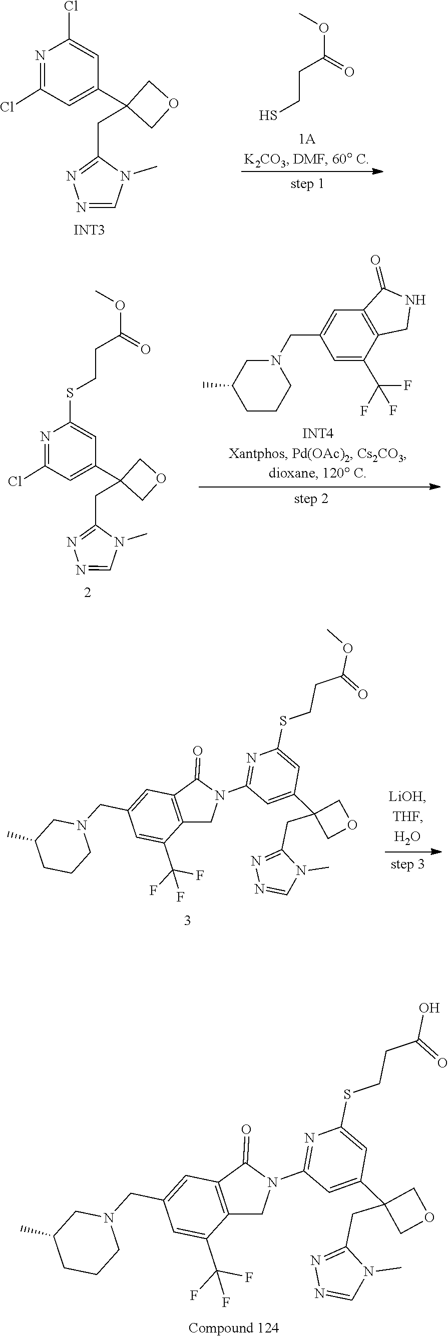

### Step 1: Synthesis of Compound 2

To a mixture of 2,6-dichloro-4-{3-[(4-methyl-1,2,4-triazol-3-yl)methyl]oxetan-3-yl}pyridine (INT3, 500 mg, 1.67 mmol, 1 equiv), methyl 3-mercaptopropionate (Compound 1A, 301 mg, 2.50 mmol, 1.5 equiv) and K₂CO₃ (461 mg, 3.34 mmol, 2 equiv) in DMF (8 ml) was stirred for 3 h at 60° C. under nitrogen atmosphere. LCMS indicated the reaction was completed. The reaction was cooled down to room temperature and quenched by the addition of Water (50 mL) at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by reverse flash chromatography with the following conditions: column, silica gel; mobile phase, ACN in water (0.05% TFA), 5% to 50% gradient in 30 min; detector, UV 254 nm. This resulted in methyl 3-[(6-chloro-4-{3-[(4-methyl-1,2,4-triazol-3-yl)methyl]oxetan-3-yl}pyridin-2-yl)sulfanyl]propanoate (Compound 2, 300 mg, 47%) as a yellow oil. LCMS (ES, m/z): 383,385[M+H]⁺

### Step 2: Synthesis of Compound 3

To a mixture of methyl 3-[(6-chloro-4-{3-[(4-methyl-1,2,4-triazol-3-yl)methyl]oxetan-3-yl}pyridin-2-yl)sulfanyl]propanoate (Compound 2, 300 mg, 0.78 mmol, 1 equiv) and 6-{[(3S)-3-methylpiperidin-1-yl]methyl}-4-(trifluoromethyl)-2,3-dihydroisoindol-1-one (INT4, 245 mg, 0.78 mmol, 1.00 equiv) in dioxane (8 mL) was added Cs₂CO₃ (510 mg, 1.56 mmol, 2 equiv), XantPhos (136 mg, 0.23 mmol, 0.3 equiv) and Pd(OAc)₂ (26 mg, 0.11 mmol, 0.15 equiv) in portions at room temperature under nitrogen atmosphere. The resulting mixture was stirred for 2 h at 120° C. under nitrogen atmosphere. LCMS indicated the reaction was completed. The reaction was cooled down to room temperature and quenched by the addition of water (50 mL) at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with CH₂Cl₂/MeOH (10:1) to afford methyl 3-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)sulfanyl]propanoate (Compound 3, 170 mg, 33%) as a white solid. LCMS (ES, m/z): 659[M+H]⁺

### Step 3: Synthesis of Compound 124

To methyl 3-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)sulfanyl]propanoate (150 mg, 0.22 mmol, 1 equiv) and LiGH (10 mg, 0.45 mmol, 2 equiv) in THF (1 ml) and H₂O (1 mL) at 0° C. under nitrogen atmosphere. The resulting mixture was stirred for 1 h at room temperature under nitrogen atmosphere. LCMS indicated the reaction was completed. The residue was purified by reverse flash chromatography with the following conditions: column, C18 silica gel; mobile phase, ACN in water (0.05% TFA), 5% to 50% gradient in 30 min; detector, UV 254 nm. This resulted in 3-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)sulfanyl]propanoic acid (Compound 124, 6.5 mg, 4%) as a white solid. LCMS (ES, m/z): 645 [M+H]⁺; ¹H-NMR (DMSO-d₆, 400 MHz) δ (ppm): 8.26 (s, 1H), 8.07-7.94 (m, 3H), 6.96 (s, 1H), 5.23 (s, 2H), 4.92-4.82 (m, 4H), 3.65-3.57 (m, 4H), 3.32-3.30 (m, 5H), 2.71-2.67 (m, 4H), 1.95-1.89 (m, 1H), 1.66-1.59 (m, 5H), 0.83-0.82 (m, 4H).

### Synthesis of Compound 125

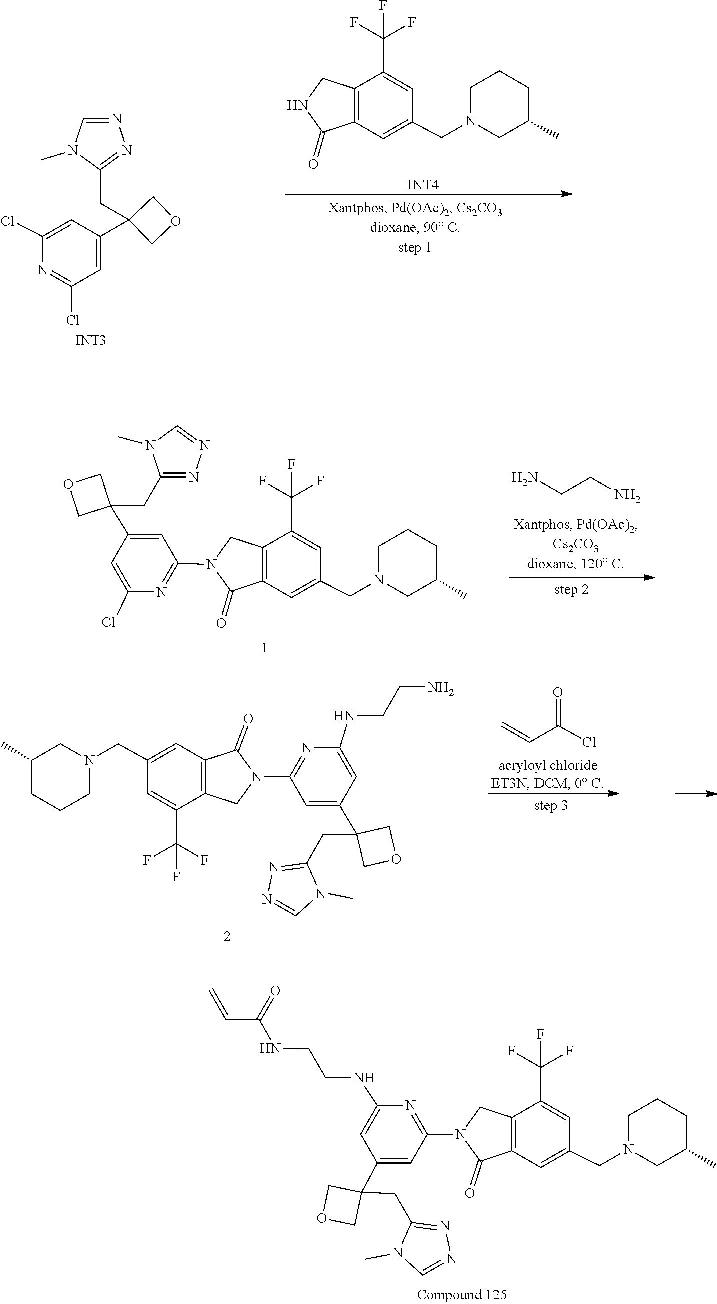

### Step 1: Synthesis of Compound 1

To a stirred mixture of 2,6-dichloro-4-(3-((4-methyl-4H-1,2,4-triazol-3-yl)methyl)oxetan-3-yl)pyridine (INT3, 235 mg, 0.786 mmol, 1 equiv) and (S)-6-((3-methylpiperidin-1-yl)methyl)-4-(trifluoromethyl)isoindolin-1-one (INT4, 245 mg, 0.786 mmol, 1 equiv) in dioxane (7 mL) was added Cs₂CO₃ (512 mg, 1.57 mmol, 2 equiv) and Xantphos (90.8 mg, 0.157 mmol, 0.2 equiv) and Pd(OAc)₂ (17.6 mg, 0.0786 mmol, 0.1 equiv) in portions at room temperature. The resulting mixture was stirred for 1 h at 90° C. under nitrogen atmosphere. The mixture was allowed to cool down to room temperature. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (3:1) to afford (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 1, 300 mg, 66.4%) as a green solid. LCMS:(ES·m/z):575,577[M+1]⁺.

### Step 2: Synthesis of Compound 2

To a stirred mixture of (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 1, 100 mg, 0.17 mmol, 1 equiv) and ethylenediamine (2.1 g, 34.7 mmol, 200 equiv) in dioxane (3 mL) was added Cs₂CO₃ (113 mg, 0.34 mmol, 2 equiv) and Xantphos (20.74 mg, 0.034 mmol, 0.2 equiv) and Pd(OAc)₂ (3.91 mg, 0.017 mmol, 0.1 equiv) in portions at room temperature. The resulting mixture was stirred for 40 min at 120° C. under nitrogen atmosphere. LCMS indicated the reaction was completed. The resulting mixture was filtered, filtrate was concentrated under reduced pressure. The reaction mixture was purified by reverse flash chromatography with the following conditions: column, C18 silica gel; mobile phase, ACN (0.1% TFA) in water (0.1% TFA), 5% to 95% gradient in 30 min; detector, UV 254 nm to afford (S)-2-(6-((2-aminoethyl)amino)-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 2, 50 mg, 48%) as a white solid. LCMS:(ES·m/z):599[M+1].

### Step 3: Synthesis of Compound 125

To a stirred mixture of (S)-2-(6-((2-aminoethyl)amino)-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 2, 25 mg, 0.042 mmol, 1 equiv) and triethylamine (4.2 mg, 0.042 mmol, 1 equiv) in dichloromethane (1.5 mL) previously cooled at 0′C was added acryloyl chloride (3.8 mg, 0.042 mmol, 1 equiv) in dichloromethane (1.5 mL). The resulting mixture was allowed to reach room temperature and stirred for 2 hours. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (3:1) to afford (S)-N-(2-((4-(3-((4-methyl-4H-1,2,4-triazol-3-yl)methyl)oxetan-3-yl)-6-(6-((3-methylpiperidin-1-yl)methyl)-1-oxo-4-(trifluoromethyl)isoindolin-2-yl)pyridin-2-yl)amino)ethyl)acrylamide (Compound 125, 15 mg, 52%) as a white solid. LCMS:(ES·m/z):653[M+1]⁺; ¹H NMR (500 MHz, DMSO-d₆) δ 8.34-8.09 (m, 3H), 7.92 (d, J=21.7 Hz, 1H), 7.41 (d, J=1.3 Hz, 1H), 6.72 (s, 1H), 6.20 (dd, J=17.1, 10.1 Hz, 1H), 6.12-5.89 (m, 2H), 5.55 (dd, J=10.1, 2.2 Hz, 1H), 5.19 (d, J=28.5 Hz, 2H), 4.89 (d, J=6.0 Hz, 2H), 4.77 (d, J=6.1 Hz, 2H), 4.49 (s, 1H), 3.63 (s, 1H), 3.48 (s, 2H), 3.27 (d, J=38.9 Hz, 22H), 3.07 (qd, J=7.3, 4.5 Hz, 4H), 2.75 (d, J=54.8 Hz, 2H), 1.92 (s, 2H), 1.86-1.45 (m, 4H), 1.18 (t, J=7.3 Hz, 7H), 0.95-0.76 (m, 4H).

### Synthesis of Compound 126

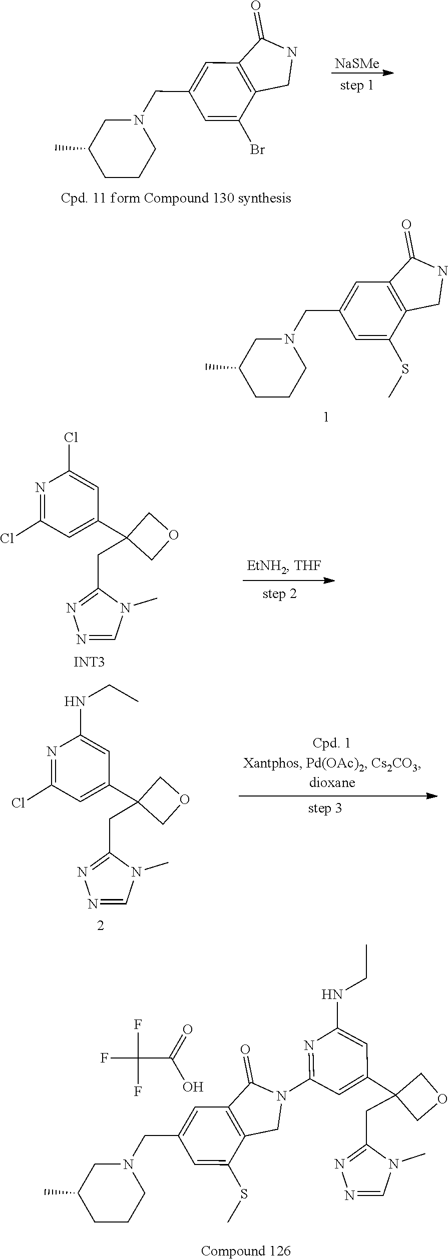

### Step 1: Synthesis of Compound 1

To a stirred solution of 4-bromo-6-{[(3S)-3-methylpiperidin-1-yl]methyl}-2,3-dihydroisoindol-1-one (Compound 11 from the synthesis of Compound 130, 200 mg, 0.61 mmol, 1 equiv) in DMF (3 mL) was added (methylsulfanyl)sodium (216 mg, 3.09 mmol, 5 equiv) dropwise at room temperature under air atmosphere. The resulting mixture was stirred for overnight at room temperature. LCMS indicated the reaction was completed. The mixture was allowed to cool down to room temperature. The reaction mixture was purified by reverse flash chromatography with the following conditions: column, C18 silica gel; mobile phase, ACN in water (0.05% TFA), 0% to 50% gradient in 40 min; detector, UV 254 nm. This resulted in 6-{[(3S)-3-methylpiperidin-1-yl]methyl}-4-(methylsulfanyl)-2,3-dihydroisoindol-1-one (Compound 1, 80 mg, 44%) as a white solid. LCMS (ES, m/z): 291 [M+H]⁺

### Step 2. Synthesis of Compound 2

To a stirred solution of 2,6-dichloro-4-{3-[(4-methyl-1,2,4-triazol-3-yl)methyl]oxetan-3-yl}pyridine (INT3, 800 mg, 2.67 mmol, 1 equiv) in ethylamine solution (2.0 M in THF, 10 mL) was stirred for overnight at 90° C. under nitrogen atmosphere. LCMS indicated the reaction was completed. The mixture was allowed to cool down to room temperature and concentrated to dryness under vacuum. The residue was purified by silica gel column chromatography, eluted with CH₂Cl₂/MeOH (10:1) to afford 6-chloro-N-ethyl-4-{3-[(4-methyl-1,2,4-triazol-3-yl)methyl]oxetan-3-yl}pyridin-2-amine (Compound 2, 450 mg, 54%) as an off-white solid. LCMS (ES, m/z): 308,310 [M+H]⁺

### Step 3: Synthesis of Compound 126

To a stirred mixture of 6-chloro-N-ethyl-4-{3-[(4-methyl-1,2,4-triazol-3-yl)methyl]oxetan-3-yl}pyridin-2-amine (Compound 2, 65 mg, 0.21 mmol, 1 equiv) and 6-{[(3S)-3-methylpiperidin-1-yl]methyl}-4-(methylsulfanyl)-2,3-dihydroisoindol-1-one (Compound 1, 61 mg, 0.21 mmol, 1 equiv) in dioxane (1 mL) was added Cs₂CO₃ (137 mg, 0.42 mmol, 2 equiv) in portions and Xantphos (24 mg, 0.04 mmol, 0.2 equiv) and Pd(OAc)₂ (5 mg, 0.02 mmol, 0.1 equiv) in portions at room temperature under nitrogen atmosphere. The resulting mixture was stirred for 2 h at 120° C. under nitrogen atmosphere. LCMS indicated the reaction was completed. The mixture was allowed to cool down to room temperature. The resulting mixture was filtered, the filter cake was washed with DCM (3×20 mL). The filtrate was concentrated under reduced pressure. The residue was purified by silica gel column chromatography, eluted with CH₂Cl₂/MeOH (10:1) to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (45 mg, crude). The crude product (45 mg) was re-purified by Prep-HPLC with the following conditions (Column: XBridge Prep Phenyl OBD Column, 19*250 mm, 5 m; Mobile Phase A: Water (0.05% TFA), Mobile Phase B: ACN; Flow rate: 25 mL/min; Gradient: 25% B to 55% B in 7 min, 55% B; Wave Length: 254 nm; RT1 (min): 5.5; The collected fraction was lyophilized to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (Compound 126, 30.1 mg, 24%) as a yellow solid. LCMS (ES, m/z): 562 [M+H]⁺; ¹H NMR (400 MHz, DMSO-d₆) δ 9.66 (br s, 1H), 8.61 (s, 1H), 7.75-7.68 (m, 2H), 7.37 (s, 1H), 5.96 (s, 1H), 4.90-4.88 (m, 4H), 4.80-4.78 (m, 2H), 4.43-4.42 (m, 2H), 3.57 (s, 2H), 3.38-3.32 (m, 1H), 3.30-3.21 (m, 6H), 2.84-2.81 (m, 1H), 2.64-2.51 (m, 4H), 1.86-1.63 (m, 4H), 1.16-1.04 (m, 4H), 0.94-0.88 (m, 3H).

### Synthesis of Compound 127

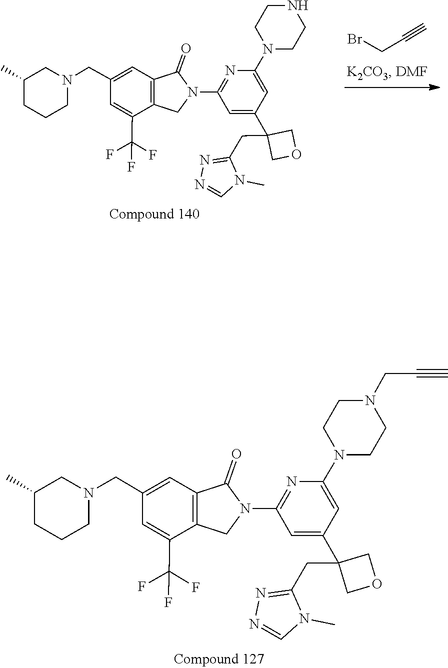

To a stirred mixture of (S)-2-(4-(3-((4-methyl-4H-1,2,4-triazol-3-yl)methyl)oxetan-3-yl)-6-(piperazin-1-yl)pyridin-2-yl)-6-((3-methylpiperidin-1-yl)methyl)-4-(trifluoromethyl)isoindolin-1-one (Compound 140, 25 mg, 0.04 mmol, 1 equiv) and K₂CO₃ (6.1 mg, 0.044 mmol, 1.1 equiv) in DIF (1 ml) was added propargyl bromide (14 mg, 0.12 mmol, 3 equiv) in DMF (1 mL). The resulting mixture was stirred for 2 hours. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (3:1) to afford (S)-2-(4-(3-((4-methyl-4H-1,2,4-triazol-3-yl)methyl)oxetan-3-yl)-6-(4-(prop-2-yn-1-yl)piperazin-1-yl)pyridin-2-yl)-6-((3-methylpiperidin-1-yl)methyl)-4-(trifluoromethyl)isoindolin-1-one (Compound 127, 15 mg, 57%) as a white solid. LCMS:(ES·m/z):663[M+1]⁺; ¹H NMR (400 MHz, Acetone) δ 8.06 (s, 1H), 7.65 (d, J=1.1 Hz, 1H), 6.13 (s, 1H), 5.23 (s, 2H), 5.02 (d, J=6.2 Hz, 2H), 4.94 (d, J=6.1 Hz, 2H), 3.60 (s, 2H), 3.55 (t, J=5.1 Hz, 4H), 3.37 (d, J=2.4 Hz, 2H), 3.22 (s, 3H), 3.16 (d, J=7.5 Hz, 5H), 2.78 (s, 6H), 2.73 (t, J=2.4 Hz, 2H), 2.61 (t, J=5.0 Hz, 4H), 1.74 (s, 1H), 1.59 (s, 1H), 1.38 (t, J=7.3 Hz, 9H), 1.29 (s, 4H), 0.13 (s, 2H).

### Synthesis of Compound 128

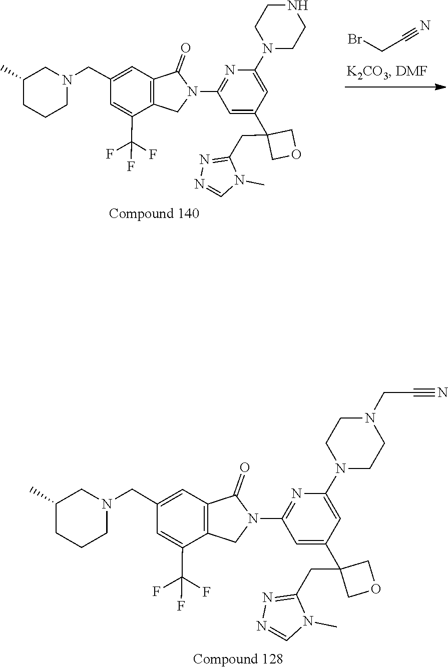

To a stirred mixture of (S)-2-(4-(3-((4-methyl-4H-1,2,4-triazol-3-yl)methyl)oxetan-3-yl)-6-(piperazin-1-yl)pyridin-2-yl)-6-((3-methylpiperidin-1-yl)methyl)-4-(trifluoromethyl)isoindolin-1-one (Compound 140, 37 mg, 0.059 mmol, 1 equiv) and K₂CO₃ (4.1 mg, 0.03 mmol, 0.5 equiv) in DMF (1 ml) was added 2-bromoacetonitrile (21 mg, 0.18 mmol, 3 equiv) in DMF (1 mL). The resulting mixture was stirred for 2 hours. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (3:1) to afford (S)-2-(4-(4-(3-((4-methyl-4H-1,2,4-triazol-3-yl)methyl)oxetan-3-yl)-6-(6-((3-methylpiperidin-1-yl)methyl)-1-oxo-4-(trifluoromethyl)isoindolin-2-yl)pyridin-2-yl)piperazin-1-yl)acetonitrile (Compound 128, 15 mg, 38%) as a white solid. LCMS:(ES·m/z):664[M+1]⁺; ¹H NMR (400 MHz, CDCl₃) δ 8.05 (s, 1H), 7.96 (s, 1H), 7.68-7.63 (m, 1H), 5.76-5.71 (m, 1H), 5.17-5.08 (m, 4H), 5.04 (d, J=6.3 Hz, 2H), 3.67 (s, 3H), 3.62-3.47 (m, 8H), 3.18-3.10 (m, 1H), 3.11 (s, 3H), 2.81 (s, 3H), 2.60 (t, J=5.0 Hz, 4H), 1.74 (s, 3H), 1.45 (t, J=7.3 Hz, 2H), 1.25 (s, 2H), 0.87 (d, J=6.0 Hz, 3H), 0.07 (s, 1H).

### Synthesis of Compound 129

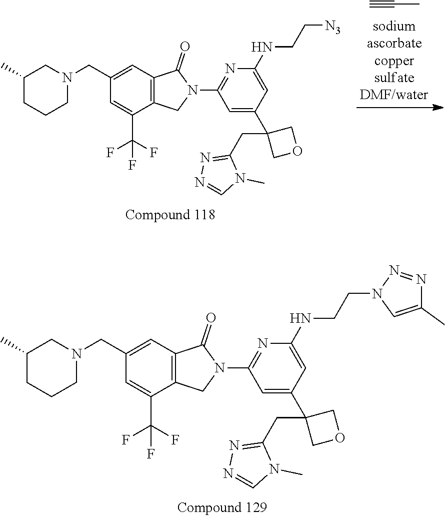

To a stirred mixture of (S)-2-(6-((2-azidoethyl)amino)-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 118, 25 mg, 0.04 mmol, 1 equiv) and propyne (24 mg, 0.6 mmol, 15 equiv) in DMF (1 ml) was added sodium ascorbate (3.2 mg, 0.016 mmol, 0.4equiv) in H₂O (0.5 ml) followed by dropwise addition of copper sulfate (6.4 mg, 0.04 mmol, 1 equiv) in H₂O (0.5 mL). The resulting mixture was stirred overnight. In the morning propyne (24 mg, 0.6 mmol, 15 equiv) was added and reaction was stirring for 1 hour. LCMS indicated the reaction was completed. The reaction was filtered, solvent removed on rotary evaporator and residue was purified by silica gel column chromatography, eluted with CH₂Cl₂/MeOH (3:1) to afford (S)-2-(6-((2-(4-methyl-1H-1,2,3-triazol-1-yl)ethyl)amino)-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 129, 12 mg, 45%) as a white solid. LCMS:(ES·m/z):665[M+1]⁺; ¹H NMR (400 MHz, DMSO-d₆) δ 8.23 (s, 1H), 7.93 (d, J=14.8 Hz, 3H), 7.74 (s, 1H), 7.45 (d, J=1.2 Hz, 2H), 6.87 (t, J=5.8 Hz, 1H), 5.94 (d, J=1.3 Hz, 1H), 5.20 (s, 2H), 5.00-4.66 (m, 4H), 4.48 (t, J=6.4 Hz, 2H), 3.65 (d, J=4.9 Hz, 5H), 3.34 (s, 173H), 3.21 (s, 4H), 2.80-2.61 (m, 4H), 2.53 (s, 14H), 2.19 (s, 4H), 1.90 (s, 4H), 1.66-0.70 (m, 22H).

### Synthesis of Compound 130

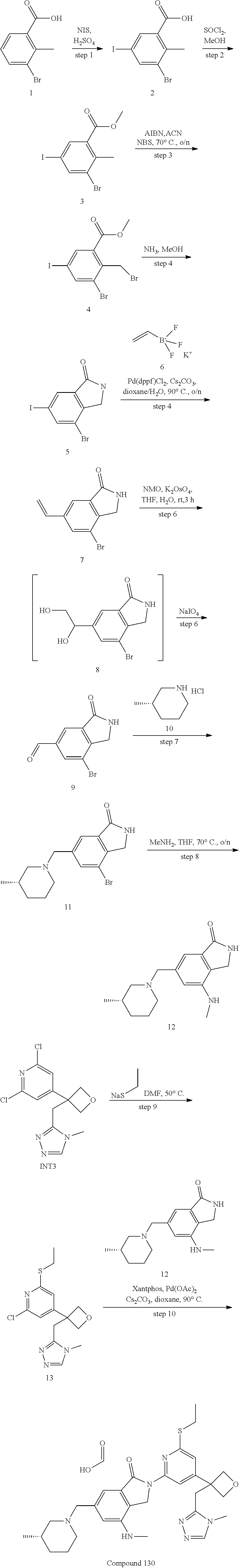

### Step 1: Synthesis of Compound 2

To a stirred solution of NIS (57.54 g, 255.75 mmol, 1.1 equiv) in H2SO₄ (200 mL) was stirred for 40 min at room temperature under nitrogen atmosphere. To the above mixture was added 3-bromo-2-methylbenzoic acid (Compound 1, 50 g, 232.50 mmol, 1 equiv) in H2SO₄ (200 mL) dropwise at 0° C. and stirred for 3 h, The resulting mixture was stirred for additional overnight at room temperature. LCMS indicated the reaction was completed. The reaction was pour into Water/Ice (1000 mL) at 0° C. The precipitated solids were collected by filtration and washed with Et₂O (3×50 mL). The residue was purified by trituration with DCM (100 mL). The precipitated solids were collected by filtration and washed with DCM (3×50 mL). This resulted in 3-bromo-5-iodo-2-methylbenzoic acid (Compound 2, 54.2 g, 68%) as an off-white solid. LCMS (ESI, ms):339[M−H]; ¹H NMR (300 MHz, DMSO-d₆) δ 8.13 (d, J=1.8 Hz, 1H), 7.99 (d, J=1.8 Hz, 1H), 2.47 (s, 3H).

### Step 2: Synthesis of Compound 3

To a stirred solution of 3-bromo-5-iodo-2-methylbenzoic acid (Compound 2, 25 g, 73.32 mmol, 1 equiv) in MeOH (250 mL) was added SOCl₂ (43.61 g, 366.63 mmol, 5 equiv) dropwise at 0° C. under nitrogen atmosphere. The resulting mixture was stirred for 3 h at 70° C. under nitrogen atmosphere. TLC indicated the reaction was completed. The resulting mixture was allowed to cool to room temperature and concentrated under reduced pressure. This resulted in methyl 3-bromo-5-iodo-2-methylbenzoate (Compound 3, 23 g, 88%) as off-white solid. ¹H NMR (400 MHz, Chloroform-d) δ 8.03 (d, J=2.0 Hz, 1H), 8.02 (d, J=2.0 Hz, 1H), 3.90 (s, 3H), 2.56 (s, 3H).

### Step 3: Synthesis of Compound 4

To a stirred solution of methyl 3-bromo-5-iodo-2-methylbenzoate (Compound 3, 15 g, 42.25 mmol, 1 equiv) and NBS (9.03 g, 50.70 mmol, 1.2 equiv) in ACN (150 mL) was added AIBN (3.47 g, 21.12 mmol, 0.5 equiv) in portions at 70° C. under nitrogen atmosphere. LCMS & TLC showed the reaction was completed. no MS signal. The resulting mixture was concentrated under reduced pressure. The residue was purified by silica gel column chromatography, eluted with PE/EA (6:1) to afford methyl 3-bromo-2-(bromomethyl)-5-iodobenzoate (Compound 4, 16.5 g, 90%) as a white solid. ¹H NMR (300 MHz, Chloroform-d) δ 8.20 (d, J=1.8 Hz, 1H), 8.11 (d, J=1.8 Hz, 1H), 5.06 (s, 2H), 1.73 (s, 3H).

### Step 4: Synthesis of Compound 5

To a stirred solution of methyl 3-bromo-5-iodo-2-methylbenzoate (Compound 4, 14.5 g, 40.84 mmol, 1 equiv) in NH₃ (7N in MeOH, 15 mL) was stirred for 1 h at room temperature under nitrogen atmosphere. LCMS indicated the reaction was completed. The resulting mixture was concentrated under vacuum. The residue was purified by trituration with water (50 mL). The precipitated solids were collected by filtration and washed with Et₂O (3×20 mL). This resulted in 4-bromo-6-iodo-2,3-dihydroisoindol-1-one (11.2 g, 81%) as a white solid. LCMS (ESI, ms):338,340[M+H]⁺

### Step 5: Synthesis of Compound 7

To a stirred solution of 4-bromo-6-iodo-2,3-dihydroisoindol-1-one (Compound 5, 11.2 g, 33.14 mmol, 1 equiv) and potassium ethenyltrifluoroboranuide (Compound 6, 3.55 g, 26.51 mmol, 0.8 equiv) in 1,4-dioxane (120 mL) and H₂O (12 mL) was added Cs₂CO₃ (21.60 g, 66.28 mmol, 2 equiv) and Pd(dppf)Cl₂ (2.43 g, 3.31 mmol, 0.1 equiv) at room temperature under nitrogen atmosphere. The resulting mixture was stirred for 1 h at 90° C. under nitrogen atmosphere. LCMS indicated the reaction was completed. The mixture was allowed to cool down to room temperature. The resulting mixture was concentrated under reduced pressure. The residue was purified by silica gel column chromatography, eluted with CH₂Cl₂/MeOH (12:1) to afford 4-bromo-6-ethenyl-2,3-dihydroisoindol-1-one (Compound 7, 3.0 g, 38%) as a brown solid. LCMS (ESI, ms):238,240[M+H]⁺

### Step 6: Synthesis of Compound 9

To a stirred solution of 4-bromo-6-ethenyl-2,3-dihydroisoindol-1-one (Compound 7, 5 g, 21.00 mmol, 1 equiv) and NMO (7.38 g, 63.00 mmol, 3 equiv) in THE (50 mL) and H₂O (25 mL) was added K₂OsO₄·2H₂O (0.77 g, 2.10 mmol, 0.1 equiv) at room temperature under nitrogen atmosphere. The resulting mixture was stirred for 3 h at room temperature under nitrogen atmosphere. To the above mixture was added NaIO₄ (22.46 g, 105.00 mmol, 5 equiv) at 0° C. The resulting mixture was stirred for additional 1 h at room temperature. LCMS indicated the reaction was completed. The reaction was quenched with Water at 0° C. Then EA (100 mL) was added. The precipitated solids were collected by filtration and washed with EtOAc (3×20 mL). This resulted in 7-bromo-3-oxo-1,2-dihydroisoindole-5-carbaldehyde (Compound 9, 4 g, 79%) as a white solid. LCMS (ESI, ms):240,242[M+H]⁺

### Step 7: Synthesis of Compound 11

To a stirred solution of 7-bromo-3-oxo-1,2-dihydroisoindole-5-carbaldehyde (Compound 9, 4 g, 16.66 mmol, 1 equiv) in DCM (80 mL) was added (3S)-3-methylpiperidine hydrochloride (Compound 10, 2.71 g, 19.99 mmol, 1.2 equiv) at room temperature under nitrogen atmosphere. The resulting mixture was stirred for 1 h at room temperature under nitrogen atmosphere. To the above mixture was added STAB (17.66 g, 83.31 mmol, 5 equiv) at room temperature. The resulting mixture was stirred for additional overnight at room temperature. LCMS indicated the reaction was completed. The resulting mixture was filtered, the filter cake was washed with CH₂Cl₂ (3×20 mL). The filtrate was concentrated under reduced pressure. The residue was purified by reverse flash chromatography with the following conditions: column, silica gel; mobile phase, MeCN in water (0.1% FA), 5% to 50% gradient in 30 min; detector, UV 254 nm. The collected fraction was concentrated to afford 4-bromo-6-{[(3S)-3-methylpiperidin-1-yl]methyl}-2,3-dihydroisoindol-1-one (Compound 11, 1.2 g, 228%) as a white solid. LCMS (ESI, ms):323,325[M+H]⁺

### Step 8: Synthesis of Compound 12

To a stirred solution of 4-bromo-6-{[(3S)-3-methylpiperidin-1-yl]methyl}-2,3-dihydroisoindol-1-one (Compound 11, 500 mg, 1.54 mmol, 1 equiv) and methylamine (2N in THF, 1.2 mL, 2.40 mmol, 1.5 equiv) in THF (5 mL) was added t-BuONa (148 mg, 1.54 mmol, 1 equiv) and tBuXPhos Pd G3 (122 mg, 0.15 mmol, 0.1 equiv) at room temperature under nitrogen atmosphere. The resulting mixture was stirred for overnight at 70° C. under nitrogen atmosphere. LCMS indicated the reaction was completed. The mixture was allowed to cool down to room temperature. The resulting mixture was filtered, the filter cake was washed with DCM (3×10 mL). The filtrate was concentrated under reduced pressure. The residue was purified by reverse flash chromatography with the following conditions: column, C18 silica gel; mobile phase, MeCN in water (0.05% TFA), 50% to 50% gradient in 30 min; detector, UV 254 nm. The collected fraction was concentrated to afford 4-(methylamino)-6-{[(3S)-3-methylpiperidin-1-yl]methyl}-2,3-dihydroisoindol-1-one (Compound 11, 200 mg, 47.29%) as an off-white solid. LCMS (ESI, ms):274[M+H]⁺

### Step 9: Synthesis of Compound 13

To a stirred solution of 2,6-dichloro-4-{3-[(4-methyl-1,2,4-triazol-3-yl)methyl]oxetan-3-yl}pyridine (INT3, 800 mg, 2.67 mmol, 1 equiv) in DMF (8 mL) was added (ethylsulfanyl)sodium (247 mg, 2.94 mmol, 1.1 equiv) at room temperature under nitrogen atmosphere. The resulting mixture was stirred for 2 h at 50° C. under nitrogen atmosphere. LCMS indicated the reaction was completed. The mixture was allowed to cool down to room temperature. The resulting mixture was extracted with EtOAc (3×50 mL). The combined organic layers were washed with water (4×50 mL), brine (100 ml), dried over anhydrous Na₂SO₄. After filtration, the filtrate was concentrated under reduced pressure. This resulted in 2-chloro-6-(ethylsulfanyl)-4-{3-[(4-methyl-1,2,4-triazol-3-yl)methyl]oxetan-3-yl}pyridine (Compound 13, 570 mg, 65%) as a yellow solid. LCMS (ESI, ms):325,326[M+H]⁺

### Step 10: Synthesis of Compound 130

To a stirred solution of 2-chloro-6-(ethylsulfanyl)-4-{3-[(4-methyl-1,2,4-triazol-3-yl)methyl]oxetan-3-yl}pyridine (Compound 13, 200 mg, 0.61 mmol, 1 equiv) and 4-(methylamino)-6-{[(3S)-3-methylpiperidin-1-yl]methyl}-2,3-dihydroisoindol-1-one (Compound 12, 168 mg, 0.61 mmol, 1 equiv) in 1,4-dioxane (4 mL) was added XantPhos (71 mg, 0.12 mmol, 0.2 equiv), Cs₂CO₃ (601 mg, 1.84 mmol, 3 equiv) and Pd(AcO)₂ (13 mg, 0.06 mmol, 0.1 equiv) at room temperature under nitrogen atmosphere. The resulting mixture was stirred for 4 h at 90° C. under nitrogen atmosphere. LCMS indicated the reaction was completed. The mixture was allowed to cool down to room temperature. The residue was purified by reverse flash chromatography with the following conditions: column, silica gel; mobile phase, MeCN in water (0.05% TFA), 5% to 50% gradient in 30 min; detector, UV 254 nm. The collected fraction was concentrated to afford 40 mg crude product as a yellow solid. The crude product (40 mg) was purified by Prep-HPLC with the following conditions (Column: Xselect CSH F-Phenyl OBD Column 19*150 mm 5 m, n; Mobile Phase A: Water (0.1% FA), Mobile Phase B: ACN; Flow rate: 60 mL/min; Gradient: 16% B to 36% B in 7 min, 36% B; Wave Length: 254 nm; RT1 (min): 6.40; The collected fraction was lyophilized to afford 2-[6-(ethylsulfanyl)-4-{3-[(4-methyl-1,2,4-triazol-3-yl)methyl]oxetan-3-yl}pyridin-2-yl]-4-(methylamino)-6-{[(3S)-3-methylpiperidin-1-yl]methyl}-3H-isoindol-1-one; formic acid (Compound 130, 17.8 mg, 4.70%) as a white solid. LCMS (ESI, ms):562[M+H−FA]⁺; ¹H NMR (400 MHz, DMSO-d₆) δ 8.27 (s, 1H), 8.21 (br s, 1H), 8.04 (s, 1H), 6.94 (s, 1H), 6.84 (d, J=1.2 Hz), 6.70 (s, 1H), 6.00 (br s, 1H), 4.90 (d, J=6.4 Hz, 2H), 4.84-4.82 (m, 4H), 3.56 (s, 2H), 3.51 (br s, 2H), 3.30 (s, 3H), 3.21-3.12 (m, 2H), 2.81-2.76 (m, 5H), 1.94-1.92 (m, 1H), 1.63-1.53 (m, 4H), 1.53-1.47 (m, 1H), 1.32 (t, J=7.2 Hz, 3H), 0.87-0.81 (m, 4H).

### Synthesis of Compound 131

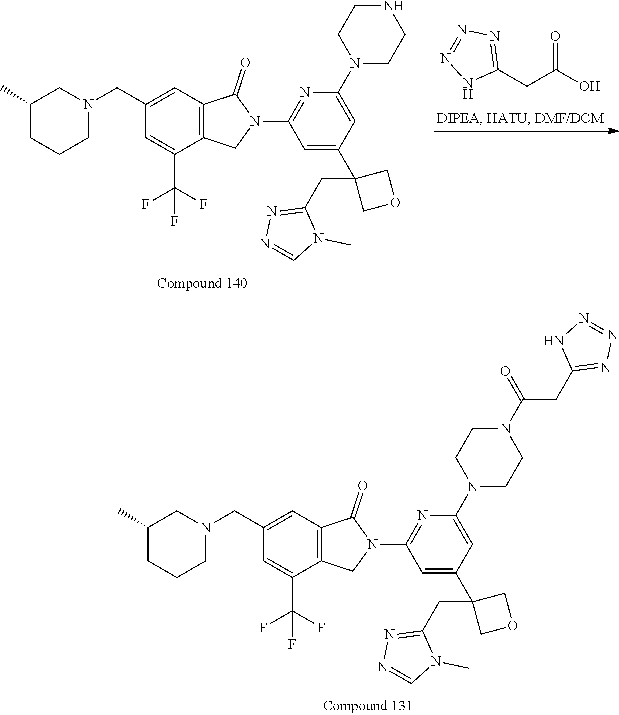 [SMILES: C[C@H]1CCCN(Cc2cc3c(c(C(F)(F)F)c2)CN(c2cc(C4(Cc5nncn5C)COC4)cc(N4CCNCC4)n2)C3=O)C1 ; O=C(O)Cc1nnn[nH]1 ; C[C@H]1CCCN(Cc2cc3c(c(C(F)(F)F)c2)CN(c2cc(C4(Cc5nncn5C)COC4)cc(N4CCN(C(=O)Cc5nnn[nH]5)CC4)n2)C3=O)C1]

To a stirred mixture of (S)-2-(4-(3-((4-methyl-4H-1,2,4-triazol-3-yl)methyl)oxetan-3-yl)-6-(piperazin-1-yl)pyridin-2-yl)-6-((3-methylpiperidin-1-yl)methyl)-4-(trifluoromethyl)isoindolin-1-one (Compound 140, 35 mg, 0.056 mmol, 1 equiv), diisopropyl ethyl amine (51 mg, 0.39 mmol, 7 equiv), HATU (32 mg, 0.084 mmol, 1.5 equiv) in 1:1 mixture of DMF/DCM (1.5 ml) was added 2-(1H-tetrazol-5-yl)acetic acid (7.2 mg, 0.056 mmol, 1 equiv) in DMF (0.5 ml). The resulting mixture was stirred for 2 hours. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (3:1) to afford (S)-2-(6-(4-(2-(1H-tetrazol-5-yl)acetyl)piperazin-1-yl)-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 131, 15 mg, 36%) as a white solid. LCMS:(ES·m/z):735[M+1]⁺; ¹H NMR (400 MHz, DMSO-d₆) δ 8.24 (s, 1H), 7.97-7.89 (m, 3H), 7.52 (d, J=1.1 Hz, 1H), 6.23 (s, 1H), 5.16 (s, 2H), 4.90 (d, J=6.2 Hz, 2H), 4.83 (d, J=6.2 Hz, 2H), 3.94-3.84 (m, 5H), 3.63 (d, J=6.3 Hz, 7H), 3.51 (s, 14H), 3.15 (s, 4H), 3.00-2.91 (m, 3H), 2.88 (s, 2H), 2.79-2.65 (m, 6H), 2.53 (s, 16H), 1.98-1.90 (m, 2H), 1.90 (s, 6H), 1.62 (s, 3H), 1.57 (s, 2H), 1.51-1.42 (m, 3H), 1.24-1.07 (m, 25H), 1.06 (d, J=1.9 Hz, 4H), 1.03 (d, J=7.0 Hz, 3H), 0.88-0.78 (m, 7H), 0.76 (s, 2H).

### Synthesis of Compound 132

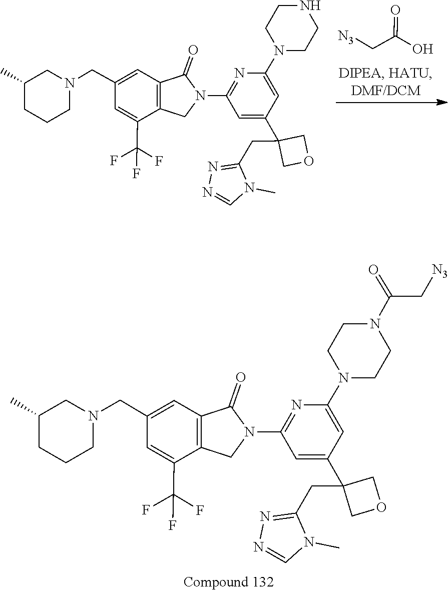

### Compound 140

To a stirred mixture of (s)-2-(4-(3-((4-methyl-4H-1,2,4-triazol-3-yl)methyl)oxetan-3-yl)-6-(piperazin-1-yl)pyridin-2-yl)-6-((3-methylpiperidin-1-yl)methyl)-4-(trifluoromethyl)isoindolin-1-one (Compound 140, 35 mg, 0.056 mmol, 1 equiv), diisopropyl ethyl amine (51 mg, 0.39 mmol, 7 equiv), HATU (32 mg, 0.084 mmol, 1.5 equiv) in 1:1 mixture of DMF/DCM (1.5 ml) was added 2-azidoacetic acid (6.8 mg, 0.067 mmol, 1,2 equiv) in DMF (0.5 ml). The resulting mixture was stirred for 2 hours. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (3:1) to afford (S)-2-(6-(4-(2-azidoacetyl)piperazin-1-yl)-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 132, 18 mg, 45%) as a white solid. LCMS:(ES·m/z):708[M+1]⁺; ¹H NMR (400 MHz, DMSO-d₆) δ 8.23 (s, 1H), 8.01-7.90 (m, 2H), 7.55 (d, J=1.0 Hz, 1H), 6.29 (d, J=1.2 Hz, 1H), 5.16 (s, 2H), 4.91 (d, J=6.2 Hz, 2H), 4.83 (d, J=6.2 Hz, 2H), 4.20 (s, 2H), 3.75 (s, 6H), 3.66 (s, 2H), 3.61-3.49 (m, 4H), 3.50 (s, 16H), 3.35 (s, 8H), 3.18 (s, 2H), 3.03 (dq, J=14.6, 7.3 Hz, 3H), 2.72 (s, 1H), 2.53 (s, 5H), 2.53 (d, J=1.3 Hz, 1H), 2.04-1.91 (m, 2H), 1.89 (s, 2H), 1.67 (s, 1H), 1.58 (s, 1H), 1.45 (s, 1H), 1.30-1.09 (m, 19H), 1.05 (s, 1H), 1.04 (t, J=7.0 Hz, 1H), 0.84 (s, 2H), 0.88-0.78 (m, 3H).

### Synthesis of Compound 133

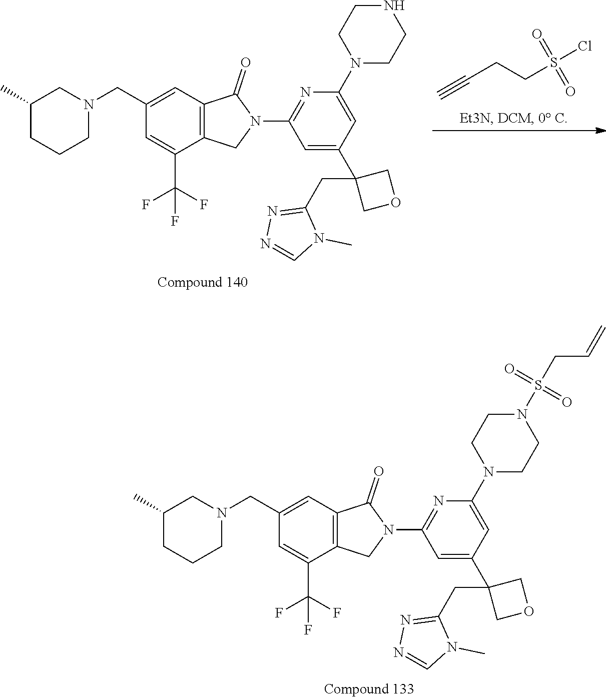

To a stirred mixture of (S)-2-(4-(3-((4-methyl-4H-1,2,4-triazol-3-yl)methyl)oxetan-3-yl)-6-(piperazin-1-yl)pyridin-2-yl)-6-((3-methylpiperidin-1-yl)methyl)-4-(trifluoromethyl)isoindolin-1-one (Compound 140, 35 mg, 0.056 mmol, 1 equiv) and triethylamine (17 mg, 0.17 mmol, 3 equiv) in dichloromethane (1.5 mL) previously cooled at 0° C. was added prop-2-ene-1-sulfonyl chloride (9.5 mg, 0.067 mmol, 1.2 equiv) in dichloromethane (1.5 mL). The resulting mixture was allowed to reach room temperature and stirred for 2 hours. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (3:1) to afford (S)-2-(6-(4-(allylsulfonyl)piperazin-1-yl)-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 133, 15 mg, 37%) as a white solid. LCMS:(ES·m/z):729 [M+1]⁺; ¹H NMR (400 MHz, DMSO-d₆) δ 8.23 (s, 4H), 5.81 (dtt, J=17.5, 10.4, 7.2 Hz, 2H), 5.58-5.30 (m, 2H), 5.19 (s, 2H), 5.09-4.91 (m, 3H), 4.91-4.76 (m, 3H), 3.98-3.86 (m, 3H), 3.54 (d, J=13.4 Hz, 8H), 3.37-3.23 (m, 86H), 3.22-3.12 (m, 7H), 3.05 (q, J=7.3 Hz, 6H), 2.53 (s, 11H), 1.18 (t, J=7.3 Hz, 13H), 0.84 (d, J=6.4 Hz, 4H).

### Synthesis of Compound 134

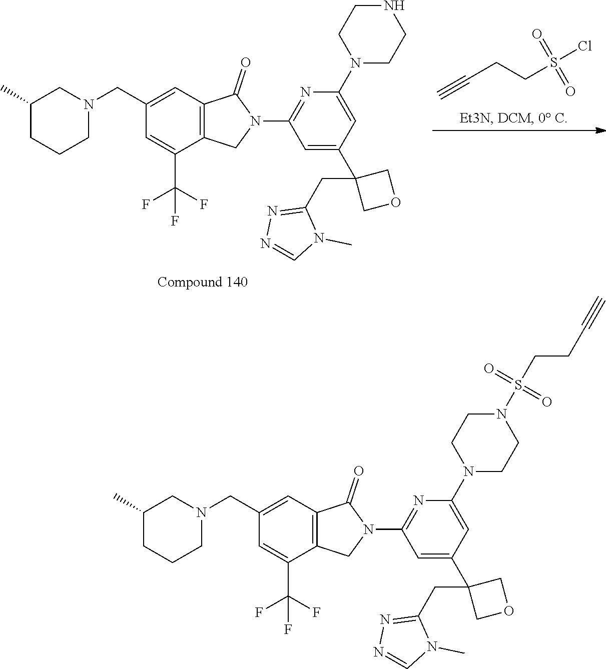

To a stirred mixture of (S)-2-(4-(3-((4-methyl-4H-1,2,4-triazol-3-yl)methyl)oxetan-3-yl)-6-(piperazin-1-yl)pyridin-2-yl)-6-((3-methylpiperidin-1-yl)methyl)-4-(trifluoromethyl)isoindolin-1-one (Compound 140, 35 mg, 0.056 mmol, 1 equiv) and triethylamine (17 mg, 0.17 mmol, 3 equiv) in dichloromethane (1.5 mL) previously cooled at 0° C. was added but-3-yne-1-sulfonyl chloride (10 mg, 0.067 mmol, 1.2 equiv) in dichloromethane (1.5 mL). The resulting mixture was allowed to reach room temperature and stirred for 2 hours. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (3:1) to afford (S)-2-(6-(4-(but-3-yn-1-ylsulfonyl)piperazin-1-yl)-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 134, 12 mg, 29%) as a white solid. LCMS:(ES·m/z):741 [M+1]⁺; ¹H NMR (400 MHz, DMSO-d₆) δ 8.23 (s, 1H), 7.94 (d, J=15.3 Hz, 2H), 7.57 (d, J=1.0 Hz, 1H), 6.37-6.32 (m, 1H), 5.16 (s, 1H), 4.91 (d, J=6.2 Hz, 2H), 4.87-4.74 (m, 2H), 3.65 (s, 1H), 3.60-3.47 (m, 6H), 3.40-3.25 (m, 23H), 3.19 (s, 3H), 2.98 (dt, J=7.8, 2.7 Hz, 1H), 2.71 (s, 1H), 2.64-2.60 (m, 1H), 2.60-2.51 (m, 3H), 2.04-1.94 (m, 1H), 1.65 (d, J=14.5 Hz, 2H), 1.58 (s, 1H), 1.54-1.43 (m, 1H), 1.28-1.18 (m, 7H), 0.88-0.78 (m, 4H).

### Synthesis of Compound 135

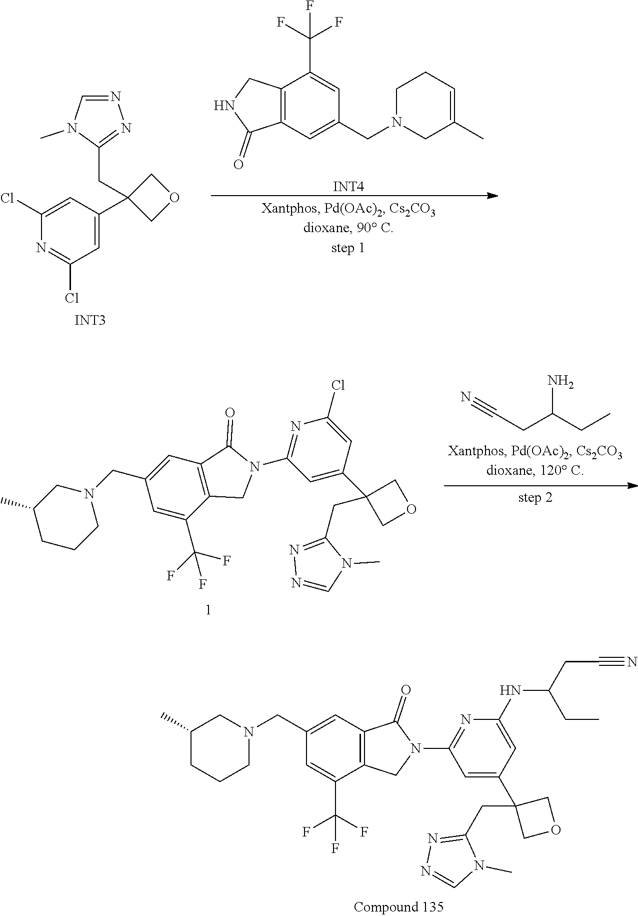

### Step 1: Synthesis of Compound 1

To a stirred mixture of 2,6-dichloro-4-(3-((4-methyl-4H-1,2,4-triazol-3-yl)methyl)oxetan-3-yl)pyridine (INT3, 235 mg, 0.786 mmol, 1 equiv) and (S)-6-((3-methylpiperidin-1-yl)methyl)-4-(trifluoromethyl)isoindolin-1-one (INT4, 245 mg, 0.786 mmol, 1 equiv) in dioxane (7 mL) was added Cs₂CO₃ (512 mg, 1.57 mmol, 2 equiv) and Xantphos (90.8 mg, 0.157 mmol, 0.2 equiv) and Pd(OAc)₂ (17.6 mg, 0.0786 mmol, 0.1 equiv) in portions at room temperature. The resulting mixture was stirred for 1 h at 90° C. under nitrogen atmosphere. The mixture was allowed to cool down to room temperature. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (3:1) to afford (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 1, 300 mg, 66.4%) as a green solid. LCMS:(ES·m/z):575,577[M+1]⁺.

### Step 2: Synthesis of Compound 135

To a stirred mixture of (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 1, 50 mg, 0.087 mmol, 1 equiv) and 3-aminopentanenitrile (43 mg, 0.43 mmol, 5 equiv) in dioxane (1.5 ml) was added Cs₂CO₃ (57 mg, 0.17 mmol, 2 equiv) and Xantphos (10 mg, 0.017 mmol, 0.2 equiv) and Pd(OAc)₂ (2 mg, 0.0087 mmol, 0.1 equiv) in portions at room temperature. The resulting mixture was stirred for 40 min at 120° C. under nitrogen atmosphere. LCMS indicated the reaction was completed. The resulting mixture was filtered, filtrate was concentrated under reduced pressure. The reaction mixture was purified by reverse flash chromatography with the following conditions: column, C18 silica gel; mobile phase, ACN in water (0.1% TFA), 5% to 95% gradient in 30 min; detector, UV 254 nm to afford 3-((4-(3-((4-methyl-4H-1,2,4-triazol-3-yl)methyl)oxetan-3-yl)-6-(6-(((S)-3-methylpiperidin-1-yl)methyl)-1-oxo-4-(trifluoromethyl)isoindolin-2-yl)pyridin-2-yl)amino)pentanenitrile; trifluoroacetic acid (Compound 135, 15 mg, 27%) as a white solid. LCMS:(ES·m/z):637[M+1]⁺; ¹H NMR (400 MHz, DMSO-d₆) δ 7.46 (s, 2H), 6.90 (d, J=7.3 Hz, 2H), 6.15 (s, 2H), 5.30 (d, J=47.5 Hz, 4H), 4.93-4.74 (m, 4H), 4.51 (s, 1H), 3.94 (d, J=6.1 Hz, 2H), 3.52 (s, 1H), 2.89 (dd, J=16.9, 5.8 Hz, 1H), 2.60-2.49 (m, 43H), 1.73 (d, J=14.1 Hz, 2H), 1.64 (t, J=8.0 Hz, 2H), 0.93 (t, J=7.3 Hz, 2H), 0.90-0.82 (m, 3H).

### Synthesis of Compound 136

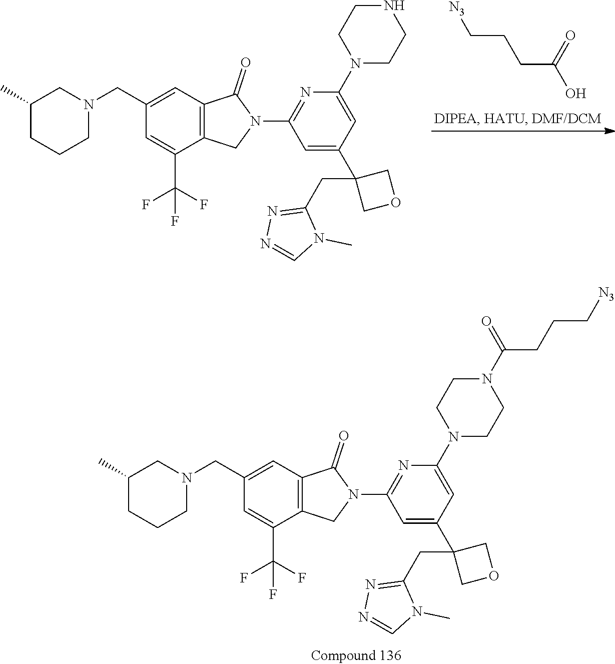 [SMILES: C[C@H]1CCCN(Cc2cc3c(c(C(F)(F)F)c2)CN(c2cc(C4(Cc5nncn5C)COC4)cc(N4CCNCC4)n2)C3=O)C1 ; [N-]=[N+]=NCCCC(=O)O ; C[C@H]1CCCN(Cc2cc3c(c(C(F)(F)F)c2)CN(c2cc(C4(Cc5nncn5C)COC4)cc(N4CCN(C(=O)CCCN=[N+]=[N-])CC4)n2)C3=O)C1]

### Compound 140

To a stirred mixture of (S)-2-(4-(3-((4-methyl-4H-1,2,4-triazol-3-yl)methyl)oxetan-3-yl)-6-(piperazin-1-yl)pyridin-2-yl)-6-((3-methylpiperidin-1-yl)methyl)-4-(trifluoromethyl)isoindolin-1-one (Compound 140, 32 mg, 0.051 mmol, 1 equiv), diisopropyl ethyl amine (46 mg, 0.36 mmol, 7 equiv), HATU (23 mg, 0.061 mmol, 1.2 equiv) in 1:1 mixture of DMF/DCM (1.5 ml) was added 4-azidobutanoic acid (7.9 mg, 0.061 mmol, 1.2 equiv) in DMF (0.5 ml). The resulting mixture was stirred for 2 hours. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (3:1) to afford (S)-2-(6-(4-(4-azidobutanoyl)piperazin-1-yl)-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 136, 12 mg, 32%) as a white solid. LCMS:(ES·m/z):736[M+1]⁺; ¹H NMR (400 MHz, DMSO) δ 8.22 (s, 1H), 7.95 (d, J=15.6 Hz, 2H), 7.55 (d, J=1.0 Hz, 1H), 6.27 (d, J=1.1 Hz, 1H), 5.16 (s, 2H), 4.91 (d, J=6.2 Hz, 2H), 4.83 (d, J=6.2 Hz, 2H), 3.59-3.49 (m, 10H), 3.46-3.40 (m, 11H), 3.34 (d, J=3.2 Hz, 10H), 3.32 (s, 7H), 3.18 (s, 3H), 2.53 (s, 7H), 2.44 (t, J=7.3 Hz, 11H), 1.77 (p, J=7.1 Hz, 3H), 1.63 (t, J=15.4 Hz, 4H), 1.19 (dt, J=23.6, 7.3 Hz, 6H), 0.81 (d, J=6.3 Hz, 5H).

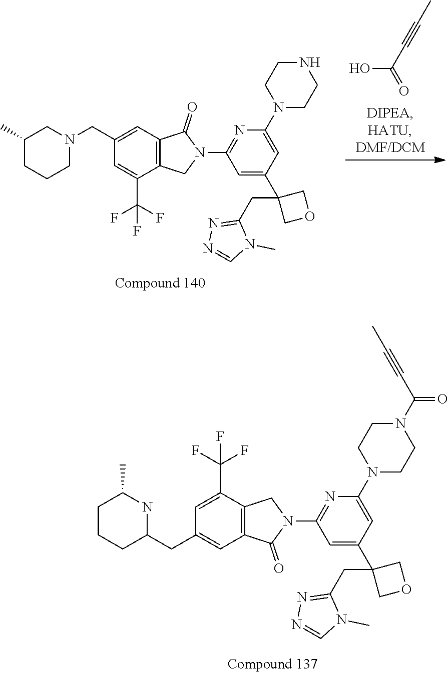

### Synthesis of Compound 137

To a stirred mixture of (S)-2-(4-(3-((4-methyl-4H-1,2,4-triazol-3-yl)methyl)oxetan-3-yl) 6-(piperazin-1-yl)pyridin-2-yl)-6-((3-methylpiperidin-1-yl)methyl)-4-(trifluoromethyl)isoindolin-1-one (Compound 140, 32 mg, 0.051 mmol, 1 equiv), diisopropyl ethyl amine (46 mg, 0.36 mmol, 7 equiv), HATU (23 mg, 0.061 mmol, 1.2 equiv) in 1:1 mixture of DMF/DCM (1.5 ml) was added but-2-ynoic acid (5.2 mg, 0.061 mmol, 1.2 equiv) in DMF (0.5 ml). The resulting mixture was stirred for 2 hours. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (3:1) to afford ((S)-2-(6-(4-(but-2-ynoyl)piperazin-1-yl)-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 137, 14 mg, 40%) as a white solid. LCMS:(ES·m/z):691[M+1]⁺; ¹H NMR (400 MHz, DMSO) δ 8.23 (s, 1H), 7.94 (d, J=15.3 Hz, 2H), 7.57 (d, J=1.0 Hz, 1H), 6.29 (d, J=1.1 Hz, 1H), 5.16 (s, 2H), 4.91 (d, J=6.2 Hz, 2H), 4.83 (d, J=6.2 Hz, 2H), 3.76 (dd, J=6.8, 3.7 Hz, 2H), 3.64 (s, 2H), 3.60-3.49 (m, 6H), 3.46 (dd, J=7.0, 3.9 Hz, 2H), 3.40-3.32 (m, 1H), 3.18 (s, 3H), 2.71 (s, 1H), 2.10-1.98 (m, 4H), 1.98-1.87 (m, 1H), 1.65-1.55 (m, 3H), 1.47 (d, J=12.1 Hz, 1H), 1.26-1.10 (m, 4H), 0.81 (d, J=6.1 Hz, 4H).

### Synthesis of Compound 138

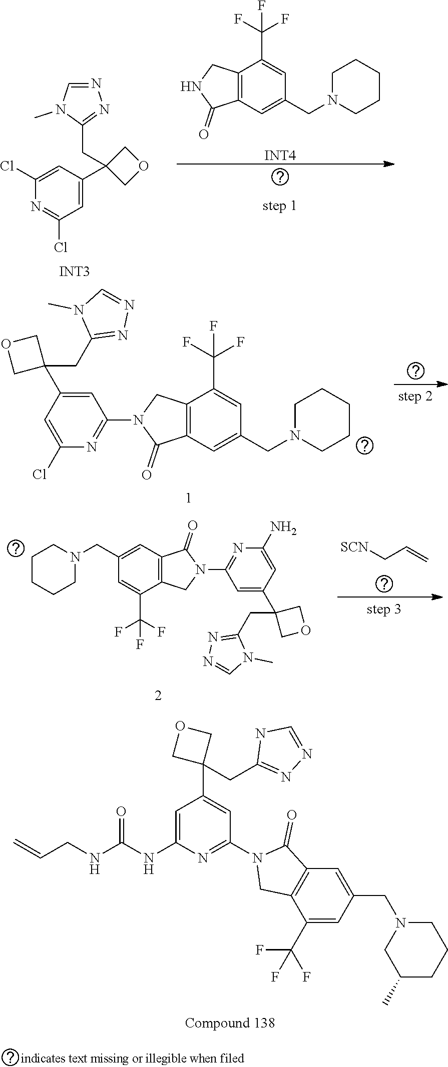

### Step 1: Synthesis of Compound 1

To a stirred mixture of 2,6-dichloro-4-(3-((4-methyl-4H-1,2,4-triazol-3-yl)methyl)oxetan-3-yl)pyridine (INT3, 235 mg, 0.786 mmol, 1 equiv) and (S)-6-((3-methylpiperidin-1-yl)methyl)-4-(trifluoromethyl)isoindolin-1-one (INT4, 245 mg, 0.786 mmol, 1 equiv) in dioxane (7 mL) was added Cs₂CO₃ (512 mg, 1.57 mmol, 2 equiv) and Xantphos (90.8 mg, 0.157 mmol, 0.2 equiv) and Pd(OAc)₂ (17.6 mg, 0.0786 mmol, 0.1 equiv) in portions at room temperature. The resulting mixture was stirred for 1 h at 90° C. under nitrogen atmosphere. The mixture was allowed to cool down to room temperature. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (9:1) to afford (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 1, 300 mg, 66.4%) as a green solid. LCMS:(ES·m/z):575,577[M+1]⁺.

### Step 2: Synthesis of Compound 2

To a stirred mixture of (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 1, 270 mg, 0.47 mmol, 1 equiv) in NH₃/dioxane (1.2 mL) was added Cs₂CO₃ (306 mg, 0.93 mmol, 2 equiv), Xantphos (54.3 mg, 0.095 mmol, 0.2 equiv) and Pd(OAc)₂ (10.5 mg, 0.047 mmol, 0.1 equiv) in portions at room temperature. The resulting mixture was stirred for 90 min at 150° C. under nitrogen atmosphere. LCMS indicated the reaction was completed. The resulting mixture was filtered, filtrate was concentrated under reduced pressure. The residue was purified by silica gel column chromatography, eluted with CH₂Cl₂/MeOH (10:1) to afford (S)-2-(6-amino-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 2, 25 mg, 52%) as a white solid. LCMS:(ES·m/z):556[M+1]⁺.

### Step 3: Synthesis of Compound 138

(S)-2-(6-amino-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 2, 30 mg, 0.054 mmol) was dissolved in EtOH (1 ml), allyl isothiocyanate (0.27 mg, 2.7 mmol, 50 eq) was added and heated in microwave initiator at 90C for 80 min. LCMS indicated the reaction was completed. Solvent was removed and product was purified on silica gel column chromatography, eluted with CH₂Cl₂/MeOH (2:1) to afford (S)-1-allyl-3-(4-(3-((4-methyl-4H-1,2,4-triazol-3-yl)methyl)oxetan-3-yl)-6-(6-((3-methylpiperidin-1-yl)methyl)-1-oxo-4-(trifluoromethyl)isoindolin-2-yl)pyridin-2-yl)thiourea (Compound 138, 7 mg, 20%) as a white solid. LCMS:(ES·m/z):655[M+1]⁺; ¹H NMR (400 MHz, CDCl₃) δ 10.98 (d, J=5.5 Hz, 1H), 9.38 (s, 1H), 8.13 (s, 1H), 8.05 (s, 1H), 7.97 (d, J=1.2 Hz, 1H), 7.89 (s, 1H), 6.64 (s, 1H), 6.12-5.98 (m, 1H), 5.58 (s, 1H), 5.40-5.28 (m, 2H), 5.23 (dd, J=10.1, 1.4 Hz, 2H), 5.11 (d, J=2.9 Hz, 4H), 5.06 (d, J=21.1 Hz, 4H), 4.48-4.41 (m, 2H), 3.67-3.57 (m, 7H), 3.38 (s, 3H), 2.78-2.69 (m, 2H), 1.96 (s, 1H), 1.48 (t, J=7.3 Hz, 4H), 1.26 (s, 9H), 1.08 (s, 1H), 0.89 (d, J=6.3 Hz, 3H), 0.86 (d, J=4.4 Hz, 7H), 0.14-0.03 (m, 8H), −0.03-−0.11 (m, 4H).

### Synthesis of Compound 140

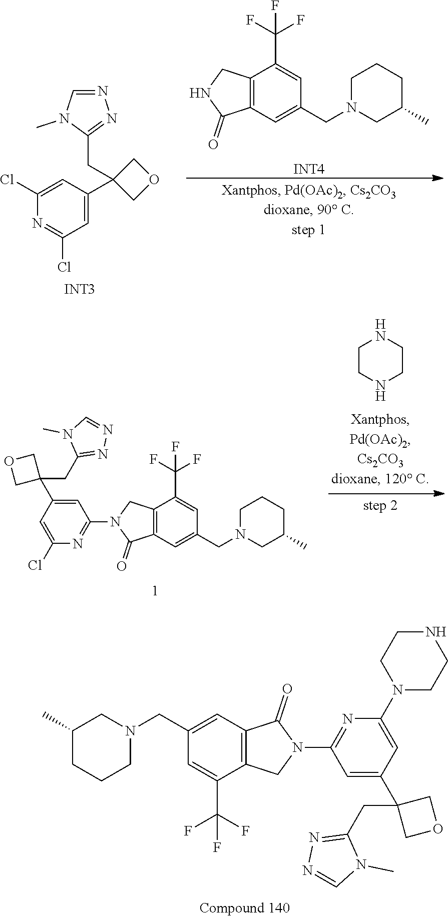

### Step 1: Synthesis of Compound 1

To a stirred mixture of 2,6-dichloro-4-(3-((4-methyl-4H-1,2,4-triazol-3-yl)methyl)oxetan-3-yl)pyridine (INT3, 235 mg, 0.786 mmol, 1 equiv) and (S)-6-((3-methylpiperidin-1-yl)methyl)-4-(trifluoromethyl)isoindolin-1-one (INT4, 245 mg, 0.786 mmol, 1 equiv) in dioxane (7 mL) was added Cs₂CO₃ (512 mg, 1.57 mmol, 2 equiv) and Xantphos (90.8 mg, 0.157 mmol, 0.2 equiv) and Pd(OAc)₂ (17.6 mg, 0.0786 mmol, 0.1 equiv) in portions at room temperature. The resulting mixture was stirred for 1 h at 90° C. under nitrogen atmosphere. The mixture was allowed to cool down to room temperature. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (3:1) to afford (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 1, 300 mg, 66.4%) as a green solid. LCMS:(ES·m/z):575,577[M+1]⁺.

### Step 2: Synthesis of Compound 140

To a stirred mixture of (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 1, 105 mg, 0.18 mmol, 1 equiv) and piperazine (157 mg, 1.83 mmol, 10 equiv) in dioxane (3.2 mL) was added Cs₂CO₃ (119 mg, 0.36 mmol, 2 equiv) and Xantphos (21.1 mg, 0.036 mmol, 0.2 equiv) and Pd(OAc)₂ (4.1 mg, 0.018 mmol, 0.1 equiv) in portions at room temperature. The resulting mixture was stirred for 40 min at 120° C. under nitrogen atmosphere. LCMS indicated the reaction was completed. The resulting mixture was filtered, filtrate was concentrated under reduced pressure. The reaction mixture was purified by reverse flash chromatography with the following conditions: column, C18 silica gel; mobile phase, ACN (0.1% TFA) in water (0.1% TFA), 5% to 95% gradient in 30 min; detector, UV 254 nm

The reaction mixture was purified by reverse flash chromatography with the following conditions: column, C18 silica gel; mobile phase, ACN in water (0.1% TFA), 5% to 95% gradient in 30 min; detector, UV 254 nm to afford (S)-2-(4-(3-((4-methyl-4H-1,2,4-triazol-3-yl)methyl)oxetan-3-yl)-6-(piperazin-1-yl)pyridin-2-yl)-6-((3-methylpiperidin-1-yl)methyl)-4-(trifluoromethyl)isoindolin-1-one; trifluoro acetic acid (Compound 140, 60 mg, 53%) as a white solid. LCMS:(ES·m/z):625[M+1]⁺; ¹H NMR (400 MHz, DMSO) δ 9.07 (s, 1H), 8.88 (s, 1H), 8.33 (s, 1H), 8.22 (d, J=17.7 Hz, 2H), 7.64 (s, 1H), 6.51 (s, 1H), 5.22 (s, 2H), 4.91 (d, J=6.2 Hz, 2H), 4.85 (d, J=6.3 Hz, 2H), 4.52 (s, 1H), 3.69 (t, J=5.2 Hz, 3H), 3.57 (s, 2H), 3.36 (d, J=11.0 Hz, 1H), 3.29 (d, J=4.9 Hz, 6H), 3.25 (s, 1H), 2.84 (d, J=11.5 Hz, 1H), 2.59 (d, J=10.1 Hz, 1H), 1.99 (q, J=7.0 Hz, 1H), 1.83 (d, J=15.0 Hz, 1H), 1.73 (d, J=13.9 Hz, 1H), 1.63 (d, J=13.6 Hz, 1H), 1.23 (s, 1H), 1.08 (dd, J=25.2, 13.9 Hz, 1H), 0.96-0.80 (m, 3H).

### Synthesis of Compound 141

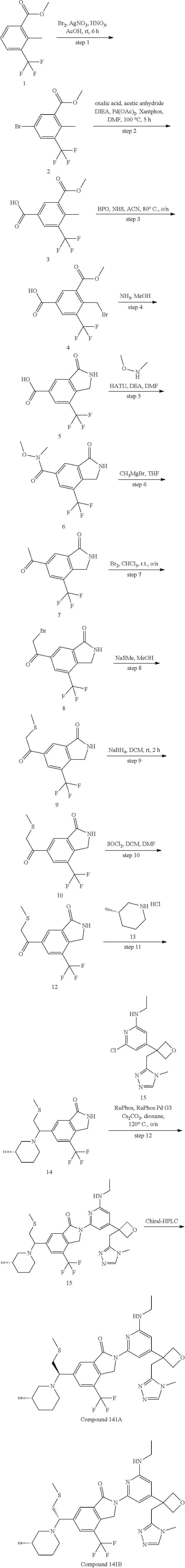

### Step 1: Synthesis of Compound 2

To a stirred mixture of methyl 2-methyl-3-(trifluoromethyl)benzoate (Compound 1, 50 g, 229.17 mmol, 1.00 equiv) in AcOH (320 mL) was added HNO₃ (222 g, 2291.74 mmol, 10 equiv, 65%) and Br₂ (40.29 g, 252.09 mmol, 1.1 equiv) and AgNO₃ (50.61 g, 297.92 mmol, 1.30 equiv) at room temperature. The resulting mixture was stirred for 6 h at room temperature. TLC indicated the reaction was completed. The mixture was basified to pH 8 with saturated NaHCO₃ (aq.). The resulting mixture was extracted with EtOAc (3×400 mL). The combined organic layers were washed with water (400 mL), dried over anhydrous Na₂SO₄. After filtration, the filtrate was concentrated under reduced pressure. The residue was purified by silica gel column chromatography, eluted with PE/EA (10:1) to afford methyl 5-bromo-2-methyl-3-(trifluoromethyl)benzoate (Compound 2, 57 g, 83%) as a colorless oil. ¹H NMR (300 MHz, DMSO-d₆) δ 8.08 (s, 1H), 7.97 (s, 1H), 3.87 (s, 3H), 2.45 (s, 3H).

### Step 2: Synthesis of Compound 3

To a stirred mixture of methyl 5-bromo-2-methyl-3-(trifluoromethyl)benzoate (Compound 2, 50 g, 168.31 mmol, 1.00 equiv) and acetic anhydride (25.77 g, 252.46 mmol, 1.50 equiv) in DMF (500.05 mL) was added oxalic acid (23.64 g, 262.56 mmol, 1.56 equiv) and Pd(OAc)₂ (3.78 g, 16.83 mmol, 0.10 equiv) and Xantphos (19.48 g, 33.66 mmol, 0.20 equiv) in portions at room temperature. The mixture was added DIEA (50.13 mL, 287.81 mmol, 1.71 equiv) dropwise at room temperature. The resulting mixture was stirred for 4 h at 100 degrees C. under nitrogen atmosphere. The mixture was allowed to cool down to room temperature. LCMS indicated the reaction was completed. The resulting mixture was quenched with water and extracted with EtOAc (3×800 mL). The combined organic layers were washed with brine (3×500 mL), dried over anhydrous Na₂SO₄. After filtration, the filtrate was concentrated under reduced pressure. The residue was purified by silica gel column chromatography, eluted with PE/EA (1:1) to afford 3-(methoxycarbonyl)-4-methyl-5-(trifluoromethyl)benzoic acid (Compound 3, 27 g, 61%) as an off-white solid. LCMS:(ES·m/z):261[M−1]⁻.

### Step 3: Synthesis of Compound 4

To a stirred mixture of 3-(methoxycarbonyl)-4-methyl-5-(trifluoromethyl)benzoic acid (Compound 3, 13.5 g, 51.49 mmol, 1.00 equiv) in CH₃CN (270 mL) were added BPO (6.60 g, 25.74 mmol, 0.5 equiv) and NBS (13.75 g, 77.23 mmol, 1.5 equiv) in portions at room temperature under nitrogen atmosphere. The resulting mixture was stirred for 5 h at 80 degrees C. under nitrogen atmosphere. LCMS indicated the reaction was completed. The mixture was allowed to cool down to room temperature. The resulting mixture was concentrated under reduced pressure. The residue was purified by silica gel column chromatography, eluted with CH₂Cl₂/MeOH (10:1) to afford 4-(bromomethyl)-3-(methoxycarbonyl)-5-(trifluoromethyl)benzoic acid (Compound 4, 10 g, 56%) as an off-white solid. LCMS:(ES·m/z):339[M−1]⁻.

### Step 4: Synthesis of Compound 5

A mixture of 4-(bromomethyl)-3-(methoxycarbonyl)-5-(trifluoromethyl)benzoic acid (Compound 4, 12 g, 35.18 mmol, 1 equiv) in NH₃ (g) in MeOH (10 M, 220 mL) was stirred for 16 h at room temperature. LCMS indicated the reaction was completed. The resulting mixture was concentrated under reduced pressure. This resulted in 3-oxo-7-(trifluoromethyl)-1,2-dihydroisoindole-5-carboxylic acid (8 g, 92%) as a yellow solid. LCMS (ESI, m/z): 244 [M−H]⁻

### Step 5: Synthesis of Compound 6

To a stirred mixture of 3-oxo-7-(trifluoromethyl)-1,2-dihydroisoindole-5-carboxylic acid (Compound 5, 7.8 g, 31.81 mmol, 1 equiv) and HATU (18.15 g, 47.72 mmol, 1.5 equiv) in DMF (150 mL) was added N,O-dimethylhydroxylamine (2.33 g, 38.17 mmol, 1.2 equiv) and DIEA (12.34 g, 95.44 mmol, 3 equiv) at 0° C. The resulting mixture was stirred for 3 h at room temperature. The reaction was quenched by the addition of Water (100 mL) at room temperature. The resulting mixture was extracted with EtOAc (3×150 mL). The combined organic layers were washed with brine (3×100 mL), dried over anhydrous Na₂SO₄. After filtration, the filtrate was concentrated under reduced pressure. The residue was purified by silica gel column chromatography, eluted with CH₂Cl₂/MeOH (10:1) to afford N-methoxy-N-methyl-3-oxo-7-(trifluoromethyl)-1,2-dihydroisoindole-5-carboxamide (Compound 6, 1.8 g, 19%) as a yellow solid. LCMS (ESI, m/z): 287 [M−H]⁻

### Step 6: Synthesis of Compound 7

To a stirred solution of N-methoxy-N-methyl-3-oxo-7-(trifluoromethyl)-1,2-dihydroisoindole-5-carboxamide (Compound 6, 1.8 g, 6.24 mmol, 1 equiv) in THE (45 mL) was added MeMgBr (12.49 mL, 12.49 mmol, 1N in THF, 2 equiv) dropwise at 0° C. under nitrogen atmosphere. The resulting mixture was stirred for 30 min at 0° C. under nitrogen atmosphere. LCMS indicated complete reaction. The reaction was quenched with Water at 0° C. The resulting mixture was extracted with EtOAc (3×50 mL). The combined organic layers were washed with brine (100 mL), dried over anhydrous Na₂SO₄. After filtration, the filtrate was concentrated under reduced pressure. The residue was purified by silica gel column chromatography, eluted with CH₂Cl₂/MeOH (10:1) to afford 6-acetyl-4-(trifluoromethyl)-2,3-dihydroisoindol-1-one (Compound 7, 900 mg, 59%) as a pink solid. LCMS (ESI, ms):244[M+H]⁺

### Step 7: Synthesis of Compound 8

To a stirred solution of 6-acetyl-4-(trifluoromethyl)-2,3-dihydroisoindol-1-one (Compound 7, 900 mg, 3.70 mmol, 1 equiv) in CH₂Cl₃ (12 mL) was added Br₂ (709 mg, 4.44 mmol, 1.2 equiv) at room temperature under nitrogen atmosphere. The resulting mixture was stirred for overnight at room temperature under nitrogen atmosphere. LCMS indicated complete reaction. The reaction was quenched with Water/Ice at 0° C. The resulting mixture was extracted with CH₂Cl₂ (3×20 mL). The combined organic layers were washed with brine (100 mL), dried over anhydrous Na₂SO₄. After filtration, the filtrate was concentrated under reduced pressure to afford 6-(2-bromoacetyl)-4-(trifluoromethyl)-2,3-dihydroisoindol-1-one (Compound 8, 1 g, 83%) as a light yellow solid. LCMS (ESI, ms):322, 324[M+H]⁺

### Step 8: Synthesis of Compound 9

To a stirred solution of sodium methanethiolate (326 mg, 4.65 mmol, 1.5 equiv) in MeOH (15 mL) was added 6-(2-bromoacetyl)-4-(trifluoromethyl)-2,3-dihydroisoindol-1-one (Compound 8, 1 g, 3.10 mmol, 1 equiv) dropwise at 0° C. under nitrogen atmosphere. The resulting mixture was stirred for 10 min at 0° C. under nitrogen atmosphere. LCMS indicated complete reaction. The reaction was quenched with Water/Ice at 0° C. The resulting mixture was extracted with EtOAc (3×20 mL). The combined organic layers were washed with brine (20 mL), dried over anhydrous Na₂SO₄. The residue was purified by silica gel column chromatography, eluted with PE/EA (1:2) to afford 6-[2-(methylsulfanyl)acetyl]-4-(trifluoromethyl)-2,3-dihydroisoindol-1-one (Compound 9, 530 mg, 57%) as a yellow solid. LCMS (ESI, ms):290[M+H]⁺

### Step 9: Synthesis of Compound 10

To a stirred solution of 6-[2-(methylsulfanyl)acetyl]-4-(trifluoromethyl)-2,3-dihydroisoindol-1-one (Compound 9, 500 mg, 1.72 mmol, 1 equiv) in DCM (5 mL) was added NaBH₄ (130 mg, 3.45 mmol, 2 equiv) in portions at 0° C. under nitrogen atmosphere. The resulting mixture was stirred for 2 h at 0° C. under nitrogen atmosphere. LCMS indicated complete reaction. The reaction was quenched with MeOH and water at 0° C. The resulting mixture was extracted with CH₂Cl₂ (3×10 mL). The combined organic layers were washed with brine (50 mL), dried over anhydrous Na₂SO₄. After filtration, the filtrate was concentrated under reduced pressure to afford 6-[1-hydroxy-2-(methylsulfanyl)ethyl]-4-(trifluoromethyl)-2,3-dihydroisoindol-1-one (Compound 10, 400 mg, 71%) as a white solid. LCMS (ESI, ms):292[M+H]⁺

### Step 10: Synthesis of Compound 12

To a stirred mixture of 6-[1-hydroxy-2-(methylsulfanyl)ethyl]-4-(trifluoromethyl)-2,3-dihydroisoindol-1-one (Compound 20, 400 mg, 1.37 mmol, 1 equiv) in DCM (4 mL) was added SOCl₂ (300 mg, 2.52 mmol, 1.84 equiv) and DMF (2drops) dropwise at 0° C. The resulting mixture was stirred for 16 h at 25° C. The resulting mixture was concentrated under vacuum. The residue was purified by silica gel column chromatography, eluted with PE/EA (1:1) to afford 6-[1-chloro-2-(methylsulfanyl)ethyl]-4-(trifluoromethyl)-2,3-dihydroisoindol-1-one (Compound 12, 400 mg, 75%) as a yellow solid. LCMS (ES, m/z): 310,312 [M+H]⁺

### Step 11: Synthesis of Compound 14

To a stirred mixture of 6-[1-chloro-2-(methylsulfanyl)ethyl]-4-(trifluoromethyl)-2,3-dihydroisoindol-1-one (Compound 12, 400 mg, 1.29 mmol, 1 equiv) and (3S)-3-methylpiperidine hydrochloride (Compound 13, 250 mg, 1.84 mmol, 1.43 equiv) in DCM (5 mL) was added TEA (400 mg, 3.95 mmol, 3.06 equiv) at 0° C. The resulting mixture was stirred for 16 h at 45° C. LCMS indicated the reaction was completed. The reaction was quenched by the addition of Water (10 mL) at room temperature. The resulting mixture was extracted with CH₂Cl₂ (3×10 mL). The combined organic layers were washed with brine (3×15 mL), dried over anhydrous Na₂SO₄. After filtration, the filtrate was concentrated under reduced pressure. The residue was purified by silica gel column chromatography, eluted with CH₂Cl₂/MeOH (10:1) to afford 6-{1-[(3S)-3-methylpiperidin-1-yl]-2-(methylsulfanyl)ethyl}-4-(trifluoromethyl)-2,3-dihydroisoindol-1-one (Compound 14, 300 mg, 59%) as a yellow solid. LCMS (ES, m/z): 373 [M+H]⁺

### Step 12: Synthesis of Compound 141

To a stirred mixture of 6-{1-[(3S)-3-methylpiperidin-1-yl]-2-(methylsulfanyl)ethyl}-4-(trifluoromethyl)-2,3-dihydroisoindol-1-one (Compound 14, 100 mg, 0.27 mmol, 1 equiv) and 6-chloro-N-ethyl-4-{3-[(4-methyl-1,2,4-triazol-3-yl)methyl]oxetan-3-yl}pyridin-2-amine (prepared as described in WO2020210508, 90 mg, 0.29 mmol, 1.1 equiv) in dioxane (2 mL) was added Cs₂CO₃ (175 mg, 0.53 mmol, 2 equiv) and RuPhos (25 mg, 0.05 mmol, 0.2 equiv) and RuPhos Palladacycle Gen.3 (22 mg, 0.03 mmol, 0.1 equiv) in portions at room temperature. The resulting mixture was stirred for overnight at ° C. under nitrogen atmosphere. The mixture was allowed to cool down to room temperature. LCMS indicated the reaction was completed. The resulting mixture was filtered, the filter cake was washed with DCM (3×30 mL). The filtrate was concentrated under reduced pressure. The residue was purified by silica gel column chromatography, eluted with CH₂Cl₂/MeOH (10:1) to afford 2-[6-(ethylamino)-4-{3-[(4-methyl-1,2,4-triazol-3-yl)methyl]oxetan-3-yl}pyridin-2-yl]-6-{1-[(3S)-3-methylpiperidin-1-yl]-2-(methylsulfanyl)ethyl}-4-(trifluoromethyl)-3H-isoindol-1-one (Compound 15, 80 mg, 46%) as a yellow solid.

The product of 2-[6-(ethylamino)-4-{3-[(4-methyl-1,2,4-triazol-3-yl)methyl]oxetan-3-yl}pyridin-2-yl]-6-{1-[(3S)-3-methylpiperidin-1-yl]-2-(methylsulfanyl)ethyl}-4-(trifluoromethyl)-3H-isoindol-1-one (Compound 15, 80 mg, 0.12 mmol, 1 equiv) was separated by Chiral-HPLC with the following conditions (Column: CHIRAL ART Amylose-SA, 2*25 cm, 5 m; Mobile Phase A: Hex (0.5% IPAmine)--HPLC, Mobile Phase B: EtOH--HPLC; Flow rate: 20 mL/min; Gradient: 20% B to 20% B in 30 min; Wave Length: 220/254 nm; RT1 (min): 14.97; RT2 (min): 23.37; Sample Solvent: EtOH--HPLC; The first eluting isomer (RT=14.97 min) was concentrated and lyophilized to afford 2-[6-(ethylamino)-4-{3-[(4-methyl-1,2,4-triazol-3-yl)methyl]oxetan-3-yl}pyridin-2-yl]-6-[(1R)-1-[(3S)-3-methylpiperidin-1-yl]-2-(methylsulfanyl)ethyl]-4-(trifluoromethyl)-3H-isoindol-1-one (Compound 141A, 23.5 mg, 29%) as a yellow solid. LCMS (ESI, ms):644[M+H]⁺; ¹H NMR (400 MHz, DMSO-d₆) δ 8.25 (s, 1H), 7.94 (d, J=10.4 Hz, 2H), 7.40 (s, 1H), 6.64-6.61 (m, 1H), 5.89 (s, 1H), 5.17 (s, 2H), 4.91 (d, J=6.0 Hz, 2H), 4.80 (d, J=6.0 Hz, 2H), 4.02-3.98 (m, 1H), 3.49 (s, 2H), 3.24-3.14 (m, 6H), 3.12-3.02 (m, 1H), 2.85-2.81 (m, 2H), 2.01 (s, 3H), 1.85-1.81 (m, 1H), 1.55-1.35 (m, 5H), 1.14 (t, J=12 Hz, 3H), 0.78-0.68 (m, 4H).

The second eluting isomer (RT=23.37 min) was concentrated and lyophilized to afford 2-[6-(ethylamino)-4-{3-[(4-methyl-1,2,4-triazol-3-yl)methyl]oxetan-3-yl}pyridin-2-yl]-6-[(1s)-1-[(3S)-3-methylpiperidin-1-yl]-2-(methylsulfanyl)ethyl]-4-(trifluoromethyl)-3H-isoindol-1-one (24.6 mg, 30%) as a yellow solid. LCMS (ESI, ms):644[M+H]⁺; ¹H NMR (400 MHz, DMSO-d₆) δ 8.25 (s, 1H), 7.94 (d, J=10.4 Hz, 2H), 7.40 (s, 1H), 6.64-6.61 (m, 1H), 5.89 (s, 1H), 5.17 (s, 2H), 4.91 (d, J=6.0 Hz, 2H), 4.80 (d, J=6.0 Hz, 2H), 4.02-3.98 (m, 1H), 3.49 (s, 2H), 3.30-3.24 (m, 5H), 3.22-3.14 (m, 1H), 3.12-3.02 (m, 1H), 2.85-2.81 (m, 2H), 2.01 (s, 3H), 1.75-1.55 (m, 5H), 1.45-1.35 (m, 1H), 1.14 (t, J=12 Hz, 3H), 0.78-0.68 (m, 4H)

### Synthesis of Compound 142

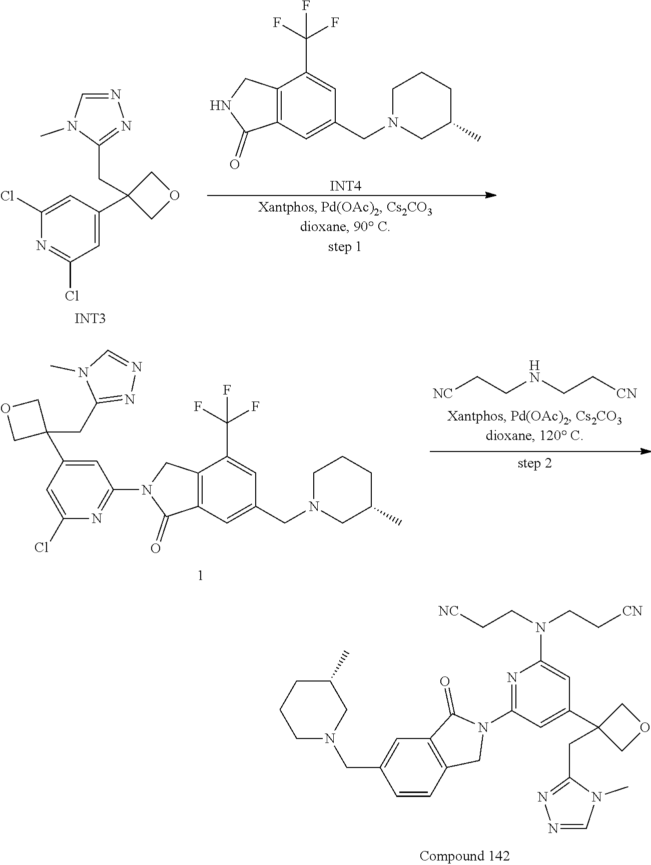

### Step 1: Synthesis of Compound 1

To a stirred mixture of 2,6-dichloro-4-(3-((4-methyl-4H-1,2,4-triazol-3-yl)methyl)oxetan-3-yl)pyridine (INT3, 235 mg, 0.786 mmol, 1 equiv) and (S)-6-((3-methylpiperidin-1-yl)methyl)-4-(trifluoromethyl)isoindolin-1-one (INT4, 245 mg, 0.786 mmol, 1 equiv) in dioxane (7 mL) was added Cs₂CO₃ (512 mg, 1.57 mmol, 2 equiv) and Xantphos (90.8 mg, 0.157 mmol, 0.2 equiv) and Pd(OAc)₂ (17.6 mg, 0.0786 mmol, 0.1 equiv) in portions at room temperature. The resulting mixture was stirred for 1 h at 90° C. under nitrogen atmosphere. The mixture was allowed to cool down to room temperature. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (9:1) to afford (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 1, 300 mg, 66.4%) as a green solid. LCMS:(ES·m/z):575,577[M+1]⁺.

### Step 2: Synthesis of Compound 142

To a stirred mixture of (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (Compound 1, 35 mg, 0.06 mmol, 1 equiv) and 3,3′-azanediyldipropanenitrile (75 mg, 0.61 mmol, 10 equiv) in dioxane (1.1 mL) was added Cs₂CO₃ (40 mg, 0.12 mmol, 2 equiv) and Xantphos (7 mg, 0.012 mmol, 0.2 equiv) and Pd(OAc)₂ (1.4 mg, 0.006 mmol, 0.1 equiv) in portions at room temperature. The resulting mixture was stirred for 40 min at 120° C. under nitrogen atmosphere. LCMS indicated the reaction was completed. The resulting mixture was filtered, filtrate was concentrated under reduced pressure. The reaction mixture was purified by reverse flash chromatography with the following conditions: column, C18 silica gel; mobile phase, ACN in water (0.1% TFA), 5% to 95% gradient in 30 min; detector, UV 254 nm to afford (S)-3,3′-((4-(3-((4-methyl-4H-1,2,4-triazol-3-yl)methyl)oxetan-3-yl)-6-(6-((3-methylpiperidin-1-yl)methyl)-1-oxo-4-(trifluoromethyl)isoindolin-2-yl)pyridin-2-yl)azanediyl)dipropanenitrile; trifluoroacetic acid (Compound 142, 15 mg, 32%) as a white solid. LCMS:(ES·m/z):662[M+1]⁺; ¹H NMR (400 MHz, DMSO-d₆) δ 9.72 (s, 1H), 8.47 (s, 2H), 8.21 (d, J=17.3 Hz, 4H), 7.55 (d, J=0.9 Hz, 2H), 6.42 (d, J=1.1 Hz, 2H), 5.75 (s, 4H), 5.30 (s, 4H), 4.95-4.73 (m, 10H), 4.52 (s, 3H), 3.84 (q, J=8.0 Hz, 7H), 3.60 (s, 4H), 3.40-3.26 (m, 10H), 3.16 (s, 2H), 2.81 (t, J=6.8 Hz, 8H), 2.59 (q, J=11.5 Hz, 3H), 2.07 (d, J=4.1 Hz, 11H), 1.83 (d, J=15.2 Hz, 3H), 1.73 (d, J=13.9 Hz, 3H), 1.63 (d, J=13.6 Hz, 2H), 1.25-0.98 (m, 6H), 0.96-0.80 (m, 8H).

### Synthesis of Compound 143

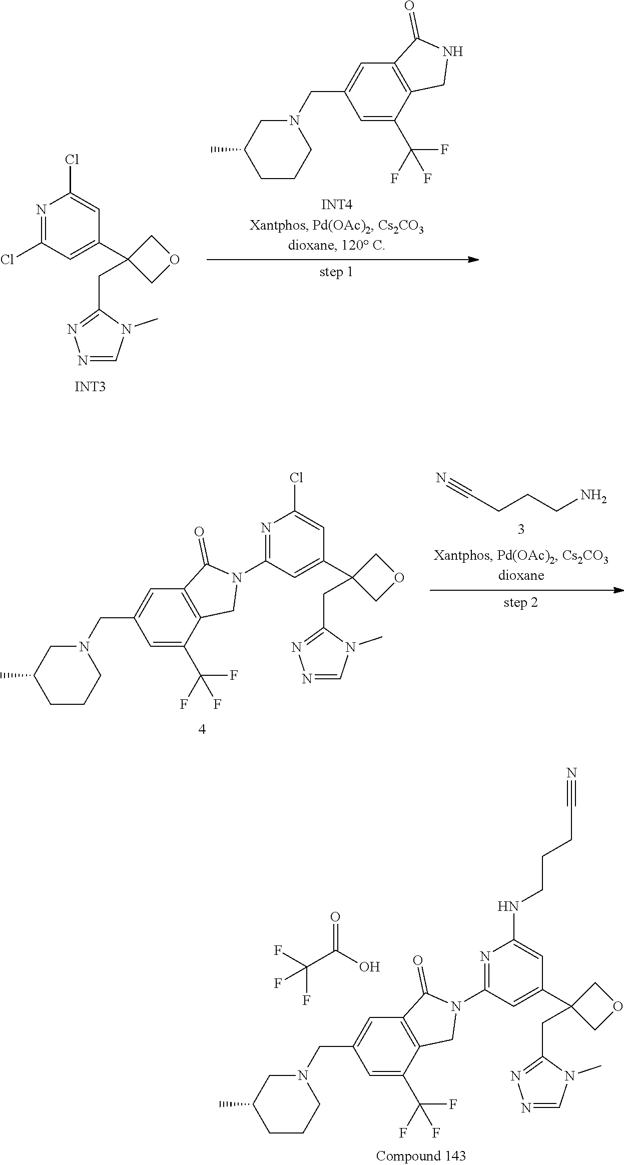

### Step 1: Synthesis of Compound 4

To a stirred mixture of 2,6-dichloro-4-{3-[(4-methyl-1,2,4-triazol-3-yl)methyl]oxetan-3-yl}pyridine (INT3, 500 mg, 1.67 mmol, 1 equiv) and 6-{[(3S)-3-methylpiperidin-1-yl]methyl}-4-(trifluoromethyl)-2,3-dihydroisoindol-1-one (INT4, 522 mg, 1.67 mmol, 1 equiv) in 1,4-dioxane (10 mL) was added Cs₂CO₃ (1089 mg, 3.34 mmol, 2 equiv) Xantphos (193 mg, 0.33 mmol, 0.2 equiv) and Pd(OAc)₂ (37 mg, 0.16 mmol, 0.1 equiv) at room temperature. The resulting mixture was stirred for 2 h at 120° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (10:1) to afford 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(trifluoromethyl)-3H-isoindol-1-one (Compound 4, 280 mg, 29%) as a green solid. LCMS (ESI, m/z): 575, 577[M+H]⁺

### Step 2: Synthesis of Compound 143

To a stirred solution of 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(trifluoromethyl)-3H-isoindol-1-one (Compound 4, 100 mg, 0.17 mmol, 1 equiv) and 4-aminobutanenitrile (Compound 3, 29 mg, 0.34 mmol, 2 equiv) in dioxane (2 mL) was added Cs₂CO₃ (113 mg, 0.34 mmol, 2 equiv), Xantphos (20 mg, 0.034 mmol, 0.2 equiv) and Pd(OAc)₂ (4 mg, 0.017 mmol, 0.1 equiv) at room temperature under nitrogen atmosphere. The resulting mixture was stirred for 2 h at 90° C. under nitrogen atmosphere. LCMS indicated the reaction was completed. The mixture was allowed to cool down to room temperature. The reaction mixture was purified by reverse flash chromatography with the following conditions: column, C18 silica gel; mobile phase, MeCN in Water (0.05% TFA), 5% to 60% gradient in 30 min; detector, UV 254 nm. The collected fraction was concentrated to afford crude product (25 mg) as a white solid. The crude product (25 mg) was re-purified by Prep-HPLC with the following conditions (Column: XSelect CSH Prep C18 OBD Column, 19*250 mm, 5 m; Mobile Phase A: Water (0.05% TFA), Mobile Phase B: ACN; Flow rate: 25 mL/min; Gradient: 20% B to 50% B in 7 min, 50% B; Wave Length: 254 nm; RT1 (min): 5; The collected fraction was lyophilized to afford 4-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]butanenitrile; trifluoroacetic acid (Compound 143, 3.1 mg, 2%) as an off-white solid. LCMS (ESI, m/z):623[M+H−TFA]⁺; ¹H NMR (300 MHz, DMSO-d₆) δ 9.86 (br s, 1H), 8.59 (s, 1H), 8.22-8.19 (m, 2H), 7.40 (s, 1H), 6.02 (s, 1H), 5.23 (s, 2H), 4.91-4.78 (m, 4H), 4.52 (s, 2H), 3.31-3.26 (m, 7H), 2.85-2.82 (m, 1H), 2.60-2.56 (m, 3H), 1.86-1.62 (m, 8H), 1.08-1.05 (m, 1H), 0.89 (d, J=5.6 Hz, 3H).

### Synthesis of Compound 144

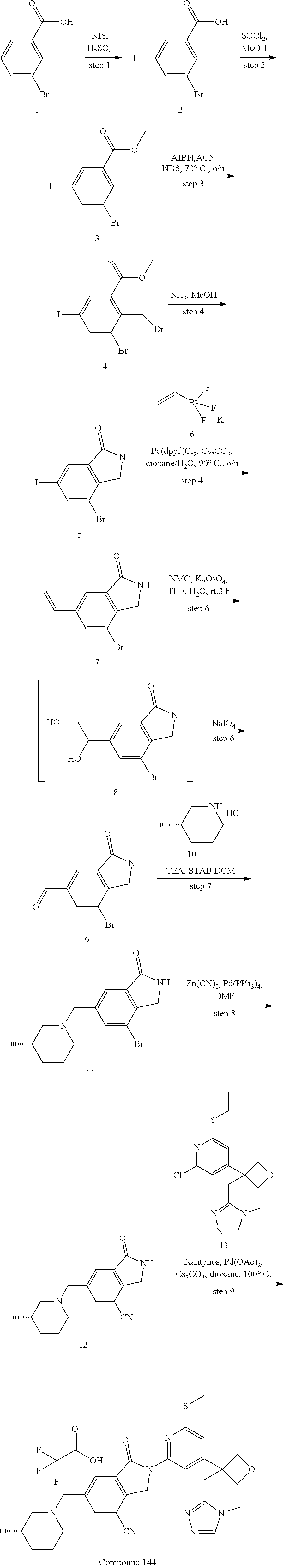

### Step 1: Synthesis of Compound 2

To a stirred solution of NIS (57.54 g, 255.75 mmol, 1.1 equiv) in H2SO₄ (200 mL) was stirred for 40 min at room temperature under nitrogen atmosphere. To the above mixture was added 3-bromo-2-methylbenzoic acid (Compound 1, 50 g, 232.50 mmol, 1 equiv) in H2SO₄ (200 mL) dropwise at 0° C. and stirred for 3 h, The resulting mixture was stirred for additional overnight at room temperature. LCMS indicated the reaction was completed. The reaction was pour into Water/Ice (1000 mL) at 0° C. The precipitated solids were collected by filtration and washed with Et₂O (3×50 mL). The residue was purified by trituration with DCM (100 mL). The precipitated solids were collected by filtration and washed with DCM (3×50 mL). This resulted in 3-bromo-5-iodo-2-methylbenzoic acid (Compound 2, 54.2 g, 68%) as an off-white solid. LCMS (ESI, ms):339[M−H]⁻; ¹H NMR (300 MHz, DMSO-d₆) δ 8.13 (d, J=1.8 Hz, 1H), 7.99 (d, J=1.8 Hz, 1H), 2.47 (s, 3H).

### Step 2: Synthesis of Compound 3

To a stirred solution of 3-bromo-5-iodo-2-methylbenzoic acid (Compound 2, 25 g, 73.32 mmol, 1 equiv) in MeOH (250 mL) was added SOCl₂ (43.61 g, 366.63 mmol, 5 equiv) dropwise at 0° C. under nitrogen atmosphere. The resulting mixture was stirred for 3 h at 70° C. under nitrogen atmosphere. TLC indicated the reaction was completed. The resulting mixture was allowed to cool to room temperature and concentrated under reduced pressure. This resulted in methyl 3-bromo-5-iodo-2-methylbenzoate (Compound 3, 23 g, 88%) as off-white solid. ¹H NMR (400 MHz, Chloroform-d) δ 8.03 (d, J=2.0 Hz, 1H), 8.02 (d, J=2.0 Hz, 1H), 3.90 (s, 3H), 2.56 (s, 3H).

### Step 3: Synthesis of Compound 4

To a stirred solution of methyl 3-bromo-5-iodo-2-methylbenzoate (15 g, 42.25 mmol, 1 equiv) and NBS (9.03 g, 50.70 mmol, 1.2 equiv) in ACN (150 mL) was added AIBN (3.47 g, 21.12 mmol, 0.5 equiv) in portions at 70° C. under nitrogen atmosphere. LCMS & TLC showed the reaction was completed. no MS signal. The resulting mixture was concentrated under reduced pressure. The residue was purified by silica gel column chromatography, eluted with PE/EA (6:1) to afford methyl 3-bromo-2-(bromomethyl)-5-iodobenzoate (16.5 g, 90%) as a white solid. ¹H NMR (300 MHz, Chloroform-d) δ 8.20 (d, J=1.8 Hz, 1H), 8.11 (d, J=1.8 Hz, 1H), 5.06 (s, 2H), 1.73 (s, 3H).

### Step 4: Synthesis of Compound 5

To a stirred solution of methyl 3-bromo-5-iodo-2-methylbenzoate (Compound 4, 14.5 g, 40.84 mmol, 1 equiv) in NH₃ (7N in MeOH, 15 mL) was stirred for 1 h at room temperature under nitrogen atmosphere. LCMS indicated the reaction was completed. The resulting mixture was concentrated under vacuum. The residue was purified by trituration with water (50 mL). The precipitated solids were collected by filtration and washed with Et₂O (3×20 mL). This resulted in 4-bromo-6-iodo-2,3-dihydroisoindol-1-one (Compound 5, 11.2 g, 81%) as a white solid. LCMS (ESI, ms):338,340[M+H]⁺

### Step 5: Synthesis of Compound 7

To a stirred solution of 4-bromo-6-iodo-2,3-dihydroisoindol-1-one (Compound 5, 11.2 g, 33.14 mmol, 1 equiv) and potassium ethenyltrifluoroboranuide (Compound 6, 3.55 g, 26.51 mmol, 0.8 equiv) in 1,4-dioxane (120 mL) and H₂O (12 mL) was added Cs₂CO₃ (21.60 g, 66.28 mmol, 2 equiv) and Pd(dppf)Cl₂ (2.43 g, 3.31 mmol, 0.1 equiv) at room temperature under nitrogen atmosphere. The resulting mixture was stirred for 1 h at 90° C. under nitrogen atmosphere. LCMS indicated the reaction was completed. The mixture was allowed to cool down to room temperature. The resulting mixture was concentrated under reduced pressure. The residue was purified by silica gel column chromatography, eluted with CH₂Cl₂/MeOH (12:1) to afford 4-bromo-6-ethenyl-2,3-dihydroisoindol-1-one (Compound 7, 3.0 g, 38%) as a brown solid. LCMS (ESI, ms):238,240[M+H]⁺

### Step 6: Synthesis of Compound 9

To a stirred solution of 4-bromo-6-ethenyl-2,3-dihydroisoindol-1-one (Compound 7, 5 g, 21.00 mmol, 1 equiv) and NMO (7.38 g, 63.00 mmol, 3 equiv) in THE (50 mL) and H₂O (25 mL) was added K₂OsO₄·2H₂O (0.77 g, 2.10 mmol, 0.1 equiv) at room temperature under nitrogen atmosphere. The resulting mixture was stirred for 3 h at room temperature under nitrogen atmosphere. To the above mixture was added NaIO₄ (22.46 g, 105.00 mmol, 5 equiv) at 0° C. The resulting mixture was stirred for additional 1 h at room temperature. LCMS indicated the reaction was completed. The reaction was quenched with Water at 0° C. Then EA (100 mL) was added. The precipitated solids were collected by filtration and washed with EtOAc (3×20 mL). This resulted in 7-bromo-3-oxo-1,2-dihydroisoindole-5-carbaldehyde (Compound 9, 4 g, 79%) as a white solid. LCMS (ESI, ms):240,242[M+H]⁺

### Step 7: Synthesis of Compound 11

To a stirred solution of 7-bromo-3-oxo-1,2-dihydroisoindole-5-carbaldehyde (Compound 9, 4 g, 16.66 mmol, 1 equiv) in DCM (80 mL) was added (3S)-3-methylpiperidine hydrochloride (Compound 10, 2.71 g, 19.99 mmol, 1.2 equiv) and TEA (2.02 g, 19.99 mmol, 1.2 equiv) at room temperature under nitrogen atmosphere. The resulting mixture was stirred for 1 h at room temperature under nitrogen atmosphere. To the above mixture was added STAB (17.66 g, 83.31 mmol, 5 equiv) at room temperature. The resulting mixture was stirred for additional overnight at room temperature. LCMS indicated the reaction was completed. The resulting mixture was filtered, the filter cake was washed with CH₂Cl₂ (3×20 mL). The filtrate was concentrated under reduced pressure. The residue was purified by reverse flash chromatography with the following conditions: column, silica gel; mobile phase, MeCN in water (0.1% FA), 5% to 50% gradient in 30 min; detector, UV 254 nm. The collected fraction was lyophilized to afford 4-bromo-6-{[(3S)-3-methylpiperidin-1-yl]methyl}-2,3-dihydroisoindol-1-one (Compound 11, 1.2 g, 22%) as a white solid. LCMS (ESI, ms):323, 325 [M+H]⁺

### Step 8: Synthesis of Compound 12

To a stirred solution of 4-bromo-6-{[(3S)-3-methylpiperidin-1-yl]methyl}-2,3-dihydroisoindol-1-one (Compound 11, 200 mg, 0.62 mmol, 1 equiv) in DMF (2 mL) was added Zn(CN)₂ (145 mg, 1.23 mmol, 2 equiv) and Pd(PPh₃)₄ (71 mg, 0.06 mmol, 0.1 equiv) in portions at room temperature under N₂ atmosphere. The resulting mixture was stirred for 2 h at 145° C. under nitrogen atmosphere. The mixture was allowed to cool down to room temperature. LCMS indicated the reaction was 60% product. The reaction mixture was purified by reverse flash chromatography with the following conditions: column, C18 silica gel; mobile phase, TFA in Water (0.05% TFA), 0% to 50% gradient in 40 min; detector, UV 254 nm. This resulted in 6-{[(3S)-3-methylpiperidin-1-yl]methyl}-1-oxo-2,3-dihydroisoindole-4-carbonitrile (Compound 12, 50 mg, 30%) as a white solid. LCMS:(ES·m/z):270[M+H]⁺.

### Step 9: Synthesis of Compound 144

To a stirred mixture of 6-{[(3S)-3-methylpiperidin-1-yl]methyl}-1-oxo-2,3-dihydroisoindole-4-carbonitrile (Compound 12, 50 mg, 0.18 mmol, 1 equiv) and 2-chloro-6-(ethylsulfanyl)-4-{3-[(4-methyl-1,2,4-triazol-3-yl)methyl]oxetan-3-yl}pyridine (Compound 2 from synthesis of Compound 107, 60 mg, 0.18 mmol, 1 equiv) in dioxane (1 mL) was added Cs₂CO₃ (120 mg, 0.37 mmol, 2 equiv) and Pd(OAc)₂ (4 mg, 0.02 mmol, 0.1 equiv) and Xantphos (21 mg, 0.04 mmol, 0.2 equiv) in portions at room temperature. The resulting mixture was stirred for 1.5 h at 110° C. under nitrogen atmosphere. The mixture was allowed to cool down to room temperature. LCMS indicated the reaction was completed. The resulting mixture was concentrated under reduced pressure. The residue was purified by reverse flash chromatography with the following conditions: column, C18 silica gel; mobile phase, TFA in Water (0.05% TFA), 0% to 50% gradient in 40 min; detector, UV 254 nm. The crude product (50 mg) was purified by Prep-HPLC with the following conditions (Column: XSelect CSH Prep C18 OBD Column, 19*250 mm, 5 m; Mobile Phase A: Water (0.05% TFA), Mobile Phase B: ACN; Flow rate: 25 mL/min; Gradient: 22% B to 42% B in 7 min, 42% B; Wave Length: 254 nm; RT1 (min): 5.38; The collected fraction was lyophilized to afford 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-1-oxo-3H-isoindole-4-carbonitrile; trifluoroacetic acid (Compound 144, 47.5 mg, 37%) as a white solid. LCMS:(ES·m/z):558[M+H−TFA]⁺; ¹H NMR (400 MHz, DMSO-d₆) δ 9.65 (br s, 1H), 8.48-8.46 (m, 1H), 8.30 (s, 2H), 8.07 (s, 1H), 7.02 (s, 1H), 5.34 (s, 2H), 4.92 (d, J=8.0 Hz, 2H), 4.85 (d, J=8.0 Hz, 2H), 4.52-4.48 (m, 2H), 3.63 (s, 2H), 3.39-3.37 (m, 4H), 3.30-3.27 (m, 1H), 3.21-3.16 (m, 2H), 2.86-2.83 (m, 1H), 2.60-2.57 (m, 1H), 1.86-1.62 (m, 4H), 1.38 (t, J=7.2 Hz, 3H), 1.08-1.05 (m, 1H), 0.89 (d, J=6.4 Hz, 1H).

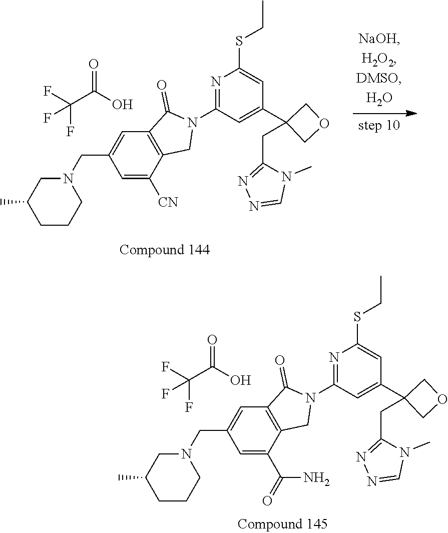

To a stirred solution of 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-1-oxo-3H-isoindole-4-carbonitrile (Compound 144, 20 mg, 0.036 mmol, 1 equiv) in DMSO (500 uL) were added NaOH (3 mg, 0.07 mmol, 2 equiv) and H₂O (200 uL) dropwise at 0° C. under air atmosphere. To the above mixture was added H₂O₂ (6 mg, 0.18 mmol, 5 equiv) dropwise at 0° C. The resulting mixture was stirred for additional 2 h at room temperature. LCMS indicated the reaction was 70% product. The reaction mixture was purified by Prep-HPLC with the following conditions (Column: XSelect CSH Prep C18 OBD Column, 19*250 mm, 5 m; Mobile Phase A: Water (0.05% TFA), Mobile Phase B: ACN; Flow rate: 25 mL/min; Gradient: 15% B to 28% B in 10 min, 28% B; Wave Length: 254 nm; RT1 (min): 8.5; The collected fraction was lyophilized to afford 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-1-oxo-3H-isoindole-4-carboxamide) (Compound 145) as a white solid. LCMS:(ES·m/z):576[M+H−TFA]⁺; ¹H NMR (400 MHz, DMSO-d₆) δ 9.63 (br s, 1H), 8.42 (s, 1H), 8.19-8.07 (m, 4H), 7.77 (s, 1H), 6.96 (s, 1H), 5.37 (s, 2H), 4.92-4.83 (m, 4H), 4.44 (br s, 2H), 3.62 (s, 2H), 3.42-3.33 (m, 5H), 3.16-3.15 (m, 2H), 2.84-2.82 (m, 1H), 2.61-2.56 (m, 1H), 1.82-1.62 (m, 4H), 1.37 (t, J=7.2 Hz, 3H), 1.08-1.05 (m, 1H), 0.90 (d, J=6.4 Hz, 1H).

### Synthesis of Compound 146

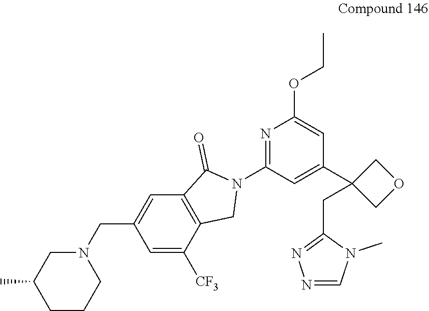 [SMILES: CCOc1cc(C2(Cc3nncn3C)COC2)cc(N2Cc3c(cc(CN4CCC[C@H](C)C4)cc3C(F)(F)F)C2=O)n1]

Compound 146 was prepared according to the procedure described in WO2020210508.

### Synthesis of Compound 147

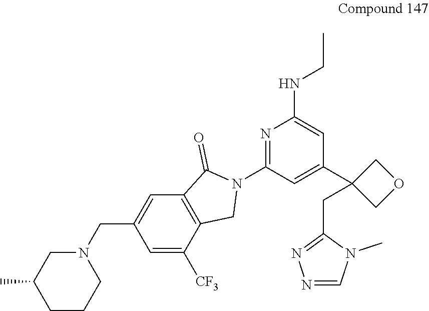 [SMILES: CCNc1cc(C2(Cc3nncn3C)COC2)cc(N2Cc3c(cc(CN4CCC[C@H](C)C4)cc3C(F)(F)F)C2=O)n1]

Compound 147 was prepared according to the procedure described in WO2020210508.

### Synthesis of Compound 148

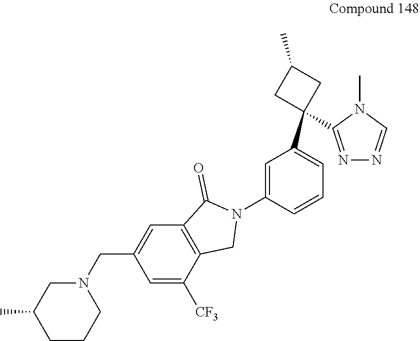 [SMILES: C[C@H]1CCCN(Cc2cc3c(c(C(F)(F)F)c2)CN(c2cccc([C@]4(c5nncn5C)C[C@@H](C)C4)c2)C3=O)C1]

Compound 148 was prepared according to the procedure described in WO2020264398.

### Synthesis of Compound 149

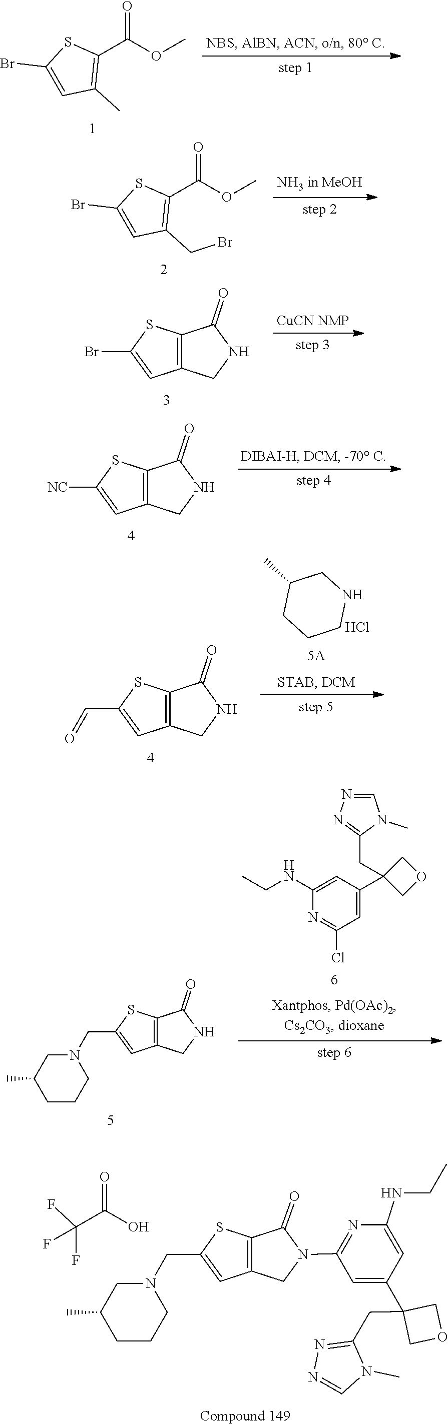

### Step 1: Synthesis of Compound 2

To a stirred solution of methyl 5-bromo-3-methylthiophene-2-carboxylate (5 g, 21.26 mmol, 1 equiv) in ACN (100 mL) were added NBS (4.16 g, 23.39 mmol, 1.1 equiv) and AIBN (0.70 g, 4.25 mmol, 0.2 equiv) in portions at room temperature under air atmosphere. The resulting mixture was stirred for overnight at 80° C. under nitrogen atmosphere. LCMS indicated the reaction was completed. The mixture was allowed to cool down to room temperature. The resulting mixture was concentrated under reduced pressure. The residue was purified by silica gel column chromatography, eluted with PE/EA (10:1) to afford methyl 5-bromo-3-(bromomethyl)thiophene-2-carboxylate (5 g, 74%) as a white solid. LCMS:(ES·m/z): 314,316[M+H]⁺.

### Step 2: Synthesis of Compound 3

To a stirred solution of methyl 5-bromo-3-(bromomethyl)thiophene-2-carboxylate (5 g, 15.92 mmol, 1 equiv) in NH₃ (7N in MeOH, 100 mL) at room temperature under air atmosphere. The resulting mixture was stirred for overnight at 70° C. under nitrogen atmosphere. The mixture was allowed to cool down to room temperature. LCMS indicated the reaction was completed. The resulting mixture was concentrated under reduced pressure. The residue was purified by reverse flash chromatography with the following conditions: column, C18 silica gel; mobile phase, TFA in Water (0.05% TFA), 0% to 50% gradient in 40 min; detector, UV 254 nm. The collected fraction was concentrated to afford 2-bromo-4H,5H-thieno[2,3-c]pyrrol-6-one (600 mg, 17%) as a yellow solid. LCMS:(ES·m/z): 218,220[M+H]⁺.

### Step 3: Synthesis of Compound 4

To a stirred solution of 2-bromo-4H,5H-thieno[2,3-c]pyrrol-6-one (400 mg, 1.83 mmol, 1 equiv) in NMP (8 mL) was added CuCN (328 mg, 3.66 mmol, 2 equiv) in portions at room temperature under nitrogen atmosphere. The resulting mixture was stirred for 2 h at 165° C. under nitrogen atmosphere. The mixture was allowed to cool down to room temperature. LCMS indicated the reaction was completed. The reaction mixture was purified by reverse flash chromatography with the following conditions: column, C18 silica gel; mobile phase, TFA in Water (0.05% TFA), 0% to 50% gradient in 30 min; detector, UV 254 nm. The collected fraction concentrated to afford 6-oxo-4H,5H-thieno[2,3-c]pyrrole-2-carbonitrile (200 mg, 66%) as a yellow solid. LCMS:(ES·m/z):165[M+H]⁺.

### Step 4: Synthesis of Compound 5

To a stirred solution of 6-oxo-4H,5H-thieno[2,3-c]pyrrole-2-carbonitrile (200 mg, 1.21 mmol, 1 equiv) in DCM (4 mL) was added Dibal-H (1M in DCM, 4.8 mL, 4 equiv) dropwise at −70° C. under nitrogen atmosphere. The resulting mixture was stirred for 2 h at −70° C. under nitrogen atmosphere. LCMS indicated the reaction was completed. The reaction was quenched by the addition of MeOH (2 mL) at 0° C. The resulting mixture was concentrated under reduced pressure. The residue was purified by reverse flash chromatography with the following conditions: column, C18 silica gel; mobile phase, TFA in Water (0.05% TFA), 0% to 50% gradient in 40 min; detector, UV 254 nm. The collected fraction concentrated to afford 6-oxo-4H,5H-thieno[2,3-c]pyrrole-2-carbaldehyde (180 mg, 88%) as a brown solid. LCMS:(ES·m/z):168[M+H]⁺.

### Step 5: Synthesis of Compound 6

To a stirred mixture of 6-oxo-4H,5H-thieno[2,3-c]pyrrole-2-carbaldehyde (200 mg, 1.19 mmol, 1 equiv) and (3S)-3-methylpiperidine hydrochloride (194 mg, 1.43 mmol, 1.2 equiv) and TEA (145 mg, 1.43 mmol, 1.2 equiv) in DCM (4 mL). The resulting mixture was stirred for 15 min at room temperature under air atmosphere. To the above mixture was added STAB (1014 mg, 4.78 mmol, 4 equiv) in portions at room temperature. The resulting mixture was stirred for additional 1 h at room temperature. LCMS indicated the reaction was completed. The reaction was quenched by the addition of MeOH (2 mL) at 0° C. The resulting mixture was concentrated under reduced pressure. The residue was purified by reverse flash chromatography with the following conditions: column, C18 silica gel; mobile phase, TFA in Water (0.05% TFA), 0% to 50% gradient in 40 min; detector, UV 254 nm. The collected fraction concentrated to afford 2-{[(3S)-3-methylpiperidin-1-yl]methyl}-4H,5H-thieno[2,3-c]pyrrol-6-one (50 mg, 16.69%) as a yellow solid. LCMS:(ES·m/z):251[M+H]⁺.

### Step 6. Synthesis of Compound 149

To a stirred mixture of 2-{[(3S)-3-methylpiperidin-1-yl]methyl}-4H,5H-thieno[2,3-c]pyrrol-6-one (50 mg, 0.20 mmol, 1 equiv) and 6-chloro-N-ethyl-4-{3-[(4-methyl-1,2,4-triazol-3-yl)methyl]oxetan-3-yl}pyridin-2-amine (55 mg, 0.18 mmol, 0.9 equiv) in dioxane (1 mL) was added Cs₂CO₃ (130 mg, 0.40 mmol, 2 equiv) and Xantphos (23 mg, 0.04 mmol, 0.2 equiv) and Pd(OAc)₂ (4 mg, 0.02 mmol, 0.1 equiv) in portions at room temperature. The resulting mixture was stirred for 1.5 h at 95° C. under nitrogen atmosphere. The mixture was allowed to cool down to room temperature. LCMS indicated the reaction was completed. The resulting mixture was concentrated under reduced pressure. The residue was purified by reverse flash chromatography with the following conditions: column, C18 silica gel; mobile phase, TFA in Water (0.05% TFA), 0% to 50% gradient in 40 min; detector, UV 254 nm. The collected fraction lyophilized to afford 5-[6-(ethylamino)-4-{3-[(4-methyl-1,2,4-triazol-3-yl)methyl]oxetan-3-yl}pyridin-2-yl]-2-{[(3S)-3-methylpiperidin-1-yl]methyl}-4H-thieno[2,3-c]pyrrol-6-one (15.6 mg, 14%) as a yellow solid. LCMS:(ES·m/z):522[M+H−TFA]⁺; ¹H NMR (400 MHz, DMSO-d₆) δ10.00 (br s, 1H), 8.76 (s, 1H), 7.50 (s, 1H), 7.18 (s, 1H), 5.95 (s, 1H), 4.98 (s, 2H), 4.88 (d, J=8 Hz, 2H), 4.80 (d, J=8 Hz, 2H), 4.65 (s, 2H), 3.68-3.58 (m, 2H), 3.45-3.22 (m, 7H), 2.85-2.80 (m, 1H), 2.56-2.52 (m, 1H), 1.87-1.60 (m, 4H), 1.17-1.02 (m, 4H), 0.93-0.85 (m, 3H).

### Synthesis of Compound 150

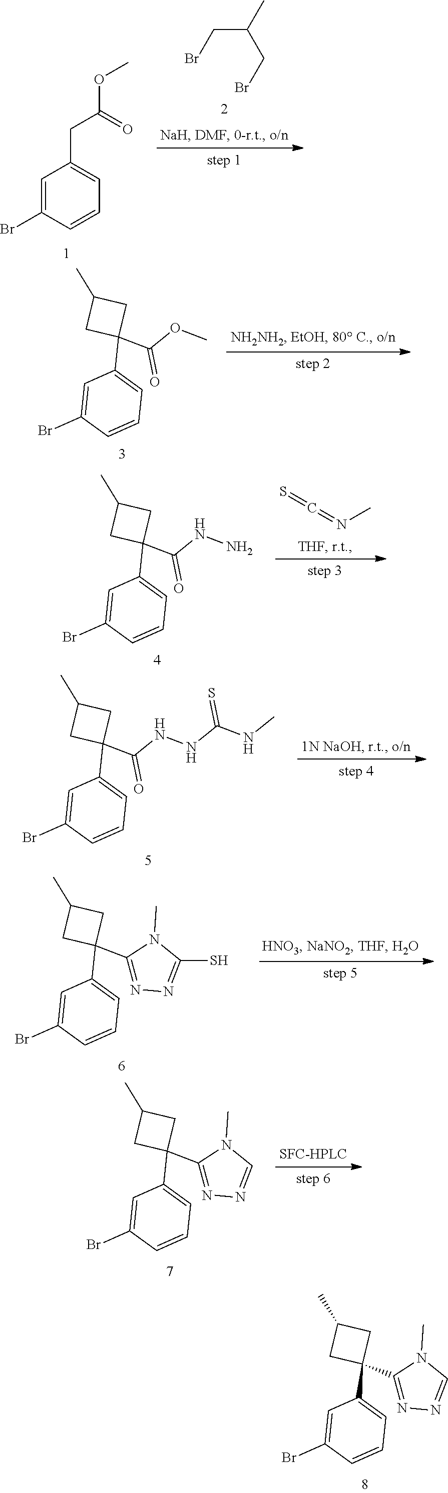

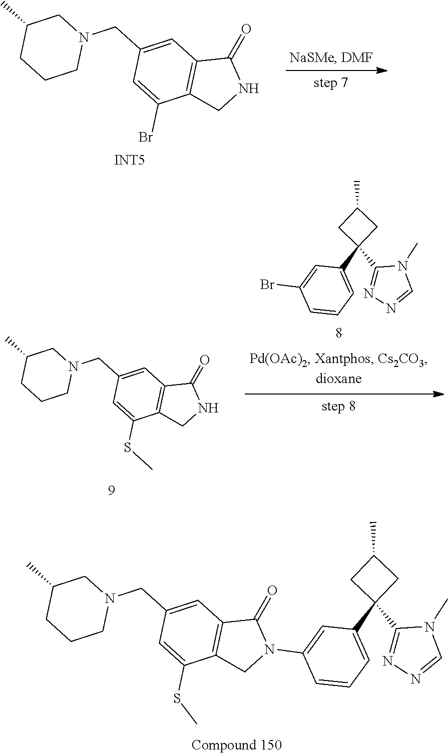

### Step 1: Synthesis of Compound 3

To a stirred mixture of methyl 2-(3-bromophenyl)acetate (48 g, 209.54 mmol, 1.00 equiv) and 1,3-dibromo-2-methylpropane (49.77 g, 230.49 mmol, 1.1 equiv) in dimethylformamide (960 mL) was added NaH (20.95 g, 513.850 mmol, 2.5 equiv, 60%) in portions slowly at 0 degrees C. under nitrogen atmosphere. The resulting mixture was stirred for overnight at room temperature under nitrogen atmosphere. LCMS indicated the reaction was completed. The reaction was quenched by the addition of sat. NH₄Cl (aq.) (2 L) at 0 degrees C. The resulting mixture was filtered, the filter cake was washed with EtOAc (4×500 mL). The filtrate was concentrated under reduced pressure. The residue was purified by silica gel column chromatography, eluted with PE/EA (50:01) to afford methyl 1-(3-bromophenyl)-3-methylcyclobutane-1-carboxylate (29 g, 48%) as a colorless oil. LCMS:(ESI, m/z): 283,285[M+H]⁺.

### Step 2: Synthesis of Compound 4

To a stirred mixture of hydrazine methyl 1-(3-bromophenyl)-3-methylcyclobutane-1-carboxylate hydrate (30 g, 90.03 mmol, 1.00 equiv) in EtOH (300 mL) was added NH₂NH₂·H₂O (39.44 g, 630.20 mmol, 7 equiv) dropwise at room temperature. The resulting mixture was stirred for 2 days at 80 degrees C. under nitrogen atmosphere. LCMS indicated the reaction was completed. The mixture was allowed to cool down to room temperature. The resulting mixture was concentrated under reduced pressure. This resulted in 1-(3-bromophenyl)-3-methylcyclobutane-1-carbohydrazide (22 g, 86%) as a colorless oil. LCMS:(ESI, m/z): 283,285[M+H]⁺.

### Step 3: Synthesis of Compound 5

To a stirred mixture of 1-(3-bromophenyl)-3-methylcyclobutane-1-carbohydrazide (30 g, 105.94 mmol, 1.00 equiv) in THE (900 mL) was added methyl isothiocyanate (23.24 g, 317.83 mmol, 3 equiv) in portions at room temperature. The resulting mixture was stirred for 3 h at 80 degrees C. under nitrogen atmosphere. LCMS indicated the reaction was completed. The mixture was allowed to cool down to room temperature. The resulting mixture was used in the next step directly without further work-ups. LCMS:(ESI, m/z): 356,358[M+H]⁺

### Step 4: Synthesis of Compound 6

To the above mixture was added NaOH (aq, 1N, 420 mL, 420 mmol, 4 equiv). The resulting mixture was stirred for 2 days at 40 degrees C. LCMS indicated the reaction was completed. The reaction was quenched with sat. NH₄Cl (aq.) (1800 mL) at 0 degrees C. The precipitated solids were collected by filtration and washed with water (2×50 mL). The solid was dried under infrared light to afford 5-[1-(3-bromophenyl)cyclobutyl]-4-methyl-1,2,4-triazole-3-thiol (22 g, two steps 61%) as an off-white solid. LCMS:(ESI·m/z): 338,340[M+H]⁺.

### Step 5: Synthesis of Compound 7

To a stirred mixture of 5-[1-(3-bromophenyl)-3-methylcyclobutyl]-4-methyl-1,2,4-triazole-3-thiol (22 g, 65.03 mmol, 1.00 equiv) in THF (22 mL) was added NaNO₂ (22.44 g, 325.15 mmol, 5 equiv) in water (22 mL) dropwise at 0 degrees C. under nitrogen atmosphere. The mixture was added HNO₃ (1N, 325 mL, 325 mmol, 5 equiv) dropwise at 0 degrees C. under nitrogen atmosphere. The resulting mixture was stirred for 3 h at 40 degrees C. LCMS indicated the reaction was completed. The mixture was basified to pH 8 with saturated NaHCO₃ (aq.). The resulting mixture was extracted with EtOAc (3×200 mL). The combined organic layers were washed with water (4×80 mL). The resulting mixture was concentrated under reduced pressure. The residue was purified by silica gel column chromatography, eluted with CH₂Cl₂/MeOH (10:1) to afford 3-[1-(3-bromophenyl)-3-methylcyclobutyl]-4-methyl-1,2,4-triazole (10 g, 50%) as an off-white solid. LCMS:(ESI·m/z): 306,308[M+H].

### Step 6: Synthesis of Compound 8

The mixture of 3-[1-(3-bromophenyl)-3-methylcyclobutyl]-4-methyl-1,2,4-triazole (10 g) was separated by Prep-SFC with the following conditions (Column: CHIRALPAK IH, 3*25 cm, m; Mobile Phase A: CO₂, Mobile Phase B: IPA (0.5% 2M NH₃-MeOH); Flow rate: 100 mL/min; Gradient: isocratic 25% B; Column Temperature (° C.): 35; Back Pressure (bar): 100; Wave Length: 220 nm; RT1 (min): 9.57; RT2 (min): 11.78; Sample Solvent: MeOH (0.1% 2M NH₃-MeOH); The first eluting isomer was concentrated and lyophilized to afford 4-methyl-3-[(1r,3s)-1-(3-bromophenyl)-3-methylcyclobutyl]-1,2,4-triazole (6 g, 60%) as an off-white solid. ¹H NMR (300 MHz, Chloroform-d) δ 8.00 (s, 1H), 7.53 (s, 1H), 7.40-7.37 (m, 1H), 7.24-7.21 (m, 2H), 3.19 (s, 3H), 2.82-2.78 (m, 2H), 2.67-2.63 (m, 3H), 1.22 (d, J=8.0 Hz, 3H).

### Step 7: Synthesis of Compound 9

To a stirred solution of 4-bromo-6-{[(3S)-3-methylpiperidin-1-yl]methyl}-2,3-dihydroisoindol-1-one (500 mg, 1.54 mmol, 1 equiv) in DMF (5 mL) was added Sodiummethanethiolate (162 mg, 2.32 mmol, 1.5 equiv) in portions at room temperature under nitrogen atmosphere. The resulting mixture was stirred for 1 h at 90° C. under nitrogen atmosphere. LCMS indicated the reaction was completed. The mixture was allowed to cool down to room temperature. The residue was purified by reverse flash chromatography with the following conditions: column, C18 silica gel; mobile phase, MeCN in Water (0.05% TFA), 5% to 60% gradient in 30 min; detector, UV 254 nm. The collected fraction was concentrated to afford 6-{[(3S)-3-methylpiperidin-1-yl]methyl}-4-(methylsulfanyl)-2,3-dihydroisoindol-1-one (410 mg, 91%) as a white solid. LCMS (ESI, m/z):291[M+H]⁺

### Step 8: Synthesis of Compound 150

To a stirred mixture of 6-{[(3S)-3-methylpiperidin-1-yl]methyl}-4-(methylsulfanyl)-2,3-dihydroisoindol-1-one (100 mg, 0.34 mmol, 1 equiv) and 4-methyl-3-[(1r,3s)-1-(3-bromophenyl)-3-methylcyclobutyl]-1,2,4-triazole (105 mg, 0.34 mmol, 1 equiv) in dioxane (2 mL) was added Pd(OAc)₂ (8 mg, 0.03 mmol, 0.1 equiv) and Cs₂CO₃ (224 mg, 0.68 mmol, 2 equiv) at room temperature under nitrogen atmosphere. The resulting mixture was stirred for 2 h at 120° C. under nitrogen atmosphere. LCMS indicated the reaction was completed. The mixture was allowed to cool down to room temperature. The reaction mixture was purified by reversed-phase flash chromatography with the following conditions: column, C18 silica gel; mobile phase, MeCN in Water (0.1% TFA), 10% to 50% gradient in 30 min; detector, UV 254 nm. The crude product (50 mg) was purified by Prep-HPLC with the following conditions (Column: XBridge Prep OBD C₁₈ Column, 30*150 mm, 5 m; Mobile Phase A: Water (10 mmol/L NH₄HCO₃), Mobile Phase B: ACN; Flow rate: 60 mL/min; Gradient: 48% B to 68% B in 7 min, 68% B; Wave Length: 254 nm; RT1 (min): 5.47; The collected fraction was lyophilized to afford 6-{[(3S)-3-methylpiperidin-1-yl]methyl}-4-(methylsulfanyl)-2-{3-[(1r,3s)-3-methyl-1-(4-methyl-1,2,4-triazol-3-yl)cyclobutyl]phenyl}-3H-isoindol-1-one (22.2 mg, 12%) as a white solid. LCMS(ESI, m/z): 516[M+H]⁺; H NMR (400 MHz, DMSO-d₆) δ 8.30 (s, 1H), 8.05 (s, 1H), 7.72-7.70 (d, J=8 Hz, 1H), 7.49-7.40 (m, 3H), 7.10-7.08 (d, J=8 Hz, 1H), 4.89 (s, 2H), 3.57 (br s, 2H), 3.22 (s, 3H), 2.87-2.74 (m, 4H), 2.60-2.53 (m, 6H), 2.01-1.89 (m, 1H), 1.68-1.51 (m, 5H), 1.10-1.08 (d, J=8 Hz, 3H), 0.83-0.82 (m, 4H)

### Synthesis of Compound 151

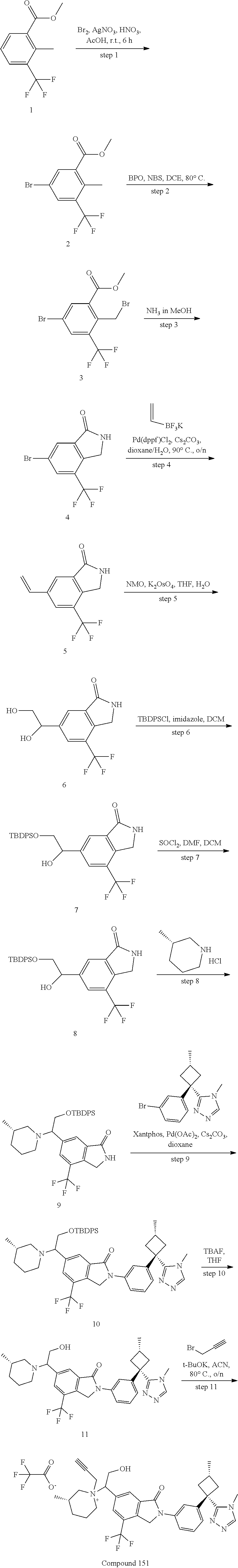

### Step 1. Synthesis of Compound 2

To a stirred solution of methyl 2-methyl-3-(trifluoromethyl)benzoate (100 g, 458.34 mmol, 1 equiv) in AcOH (650 mL) was added HNO₃ (214 mL) dropwise at 0° C. under nitrogen atmosphere. The resulting mixture was stirred for 15 min at 0° C. under nitrogen atmosphere. To the above mixture was added Br₂ (25.83 mL, 504.18 mmol, 1.1 equiv) dropwise at 0° C. The resulting mixture was stirred for additional 15 min at 0° C. To the above mixture was added AgNO₃ (101.22 g, 595.85 mmol, 1.3 equiv) in portions at 0° C. The resulting mixture was stirred for additional overnight at room temperature. GCMS and TLC indicated the reaction was completed. The resulting mixture was diluted with water (1000 mL). The resulting mixture was extracted with EtOAc (3×500 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EA (1:1) to afford methyl 5-bromo-2-methyl-3-(trifluoromethyl)benzoate (100 g, 73%) as a light yellow oil. GCMS:(ES·m/z):296,298[M]⁺.

### Step 2: Synthesis of Compound 3

To a stirred solution of methyl 5-bromo-2-methyl-3-(trifluoromethyl)benzoate (20 g, 67.32 mmol, 1.00 equiv) and NBS (17.97 g, 100.98 mmol, 1.5 equiv) in 1,2-dichloroethane (200 mL) were added BPO (3.45 g, 13.46 mmol, 0.2 equiv) at room temperature under nitrogen atmosphere. The resulting mixture was stirred for overnight at 80 degrees C. under nitrogen atmosphere. GCMS indicated complete reaction. The resulting mixture was extracted with EtOAc (3×200 mL). The combined organic layers were washed with brine (500 mL), dried over anhydrous Na₂SO₄. After filtration, the filtrate was concentrated under reduced pressure. The residue was purified by silica gel column chromatography, eluted with PE/EA (50:1) to afford methyl 5-bromo-2-(bromomethyl)-3-(trifluoromethyl)benzoate (19.7 g, 77%) as a yellow oil. ¹H NMR (300 MHz, Chloroform-d) δ 8.18 (d, J=2.1 Hz, 1H), 7.94 (d, J=2.1 Hz, 1H), 5.04 (s, 3H), 3.99 (s, 3H).

### Step 3: Synthesis of Compound 4

To a stirred solution of methyl 5-bromo-2-(bromomethyl)-3-(trifluoromethyl)benzoate (130 g, 345.77 mmol, 1 equiv) in NH₃ (7N in MeOH, 1200 mL) dropwise at 0° C. under air atmosphere. The resulting mixture was stirred for 3 h at 0° C. under nitrogen atmosphere. LCMS indicated the reaction was completed. The resulting mixture was concentrated under reduced pressure. The residue was purified by trituration with H₂O (500 mL) and PE (500 mL). This result in 6-bromo-4-(trifluoromethyl)-2,3-dihydroisoindol-1-one (70 g, 72%) as a brown solid. LCMS:(ES·m/z):280,282[M+H].

### Step 4: Synthesis of Compound 5

To a stirred mixture of 6-bromo-4-(trifluoromethyl)-2,3-dihydroisoindol-1-one (40 g, 142.83 mmol, 1 equiv) and ethenyltrifluoro-lambda4-borane potassium (22.96 g, 171.40 mmol, 1.2 equiv) in dioxane (400 mL) and H₂O (80 mL) was added Cs₂CO₃ (93.08 g, 285.67 mmol, 2 equiv) and Pd(dppf)Cl₂ (5.23 g, 7.14 mmol, 0.05 equiv) in portions at room temperature. The resulting mixture was stirred for overnight at 90° C. under nitrogen atmosphere. The mixture was allowed to cool down to room temperature. LCMS indicated the reaction was completed. The resulting mixture was filtered, the filter cake was washed with EtOAc (3×200 mL). The filtrate was concentrated under reduced pressure. The residue was purified by silica gel column chromatography, eluted with CH₂Cl₂/MeOH (10:1) to afford 6-ethenyl-4-(trifluoromethyl)-2,3-dihydroisoindol-1-one (28 g, 86%) as a yellow solid. LCMS:(ES·m/z):228[M+H]⁺.

### Step 5: Synthesis of Compound 6

To a stirred solution of 6-ethenyl-4-(trifluoromethyl)-2,3-dihydroisoindol-1-one (2.8 g, 12.32 mmol, 1 equiv) in THE (30 mL) and H₂O (15 mL) was added NMO (4.33 g, 36.97 mmol, 3 equiv) and Potassium osmate(VI) dihydrate (0.23 g, 0.61 mmol, 0.05 equiv) in portions at room temperature under air atmosphere. The resulting mixture was stirred for 2 h at room temperature under air atmosphere. LCMS indicated the reaction was completed. The resulting mixture was concentrated under reduced pressure. The residue was purified by reverse flash chromatography with the following conditions: column, C18 silica gel; mobile phase, TFA in Water (0.05% TFA), 0% to 50% gradient in 40 min; detector, UV 254 nm. The collected fraction was concentrated to afford 6-(1,2-dihydroxyethyl)-4-(trifluoromethyl)-2,3-dihydroisoindol-1-one (2.1 g, 65%) as a yellow solid. LCMS:(ES·m/z):262[M+H]⁺.

### Step 6: Synthesis of Compound 7

To a stirred solution of 6-(1,2-dihydroxyethyl)-4-(trifluoromethyl)-2,3-dihydroisoindol-1-one (9.7 g, 37.13 mmol, 1 equiv) in DCM (200 mL) was added imidazole (5.06 g, 74.27 mmol, 2 equiv) in portions and TBDPSCl (10.21 g, 37.13 mmol, 1 equiv) dropwise at room temperature under air atmosphere. The resulting mixture was stirred for 2 h at room temperature under air atmosphere. LCMS indicated the reaction was completed. The resulting mixture was diluted with water (30 mL). The resulting mixture was extracted with CH₂Cl₂ (3×40 mL). The combined organic layers were washed with brine (2×10 mL), dried over anhydrous Na₂SO₄. After filtration, the filtrate was concentrated under reduced pressure. The residue was purified by silica gel column chromatography, eluted with CH₂Cl₂/MeOH (10:1) to afford 6-{2-[(tert-butyldiphenylsilyl)oxy]-1-hydroxyethyl}-4-(trifluoromethyl)-2,3-dihydroisoindol-1-one (8.3 g, 44%) as a pink solid. LCMS:(ES·m/z):500[M+H]⁺.

### Step 7. Synthesis of Compound 8

To a stirred solution of 6-{2-[(tert-butyldiphenylsilyl)oxy]-1-hydroxyethyl}-4-(trifluoromethyl)-2,3-dihydroisoindol-1-one (8.4 g, 16.81 mmol, 1 equiv) in DCM (120 mL) was added SOCl₂ (6.00 g, 50.43 mmol, 3 equiv) dropwise at 0° C. under air atmosphere. The resulting mixture was stirred for 2 h at 0° C. under air atmosphere. LCMS indicated the reaction was 50% product. The resulting mixture was concentrated under reduced pressure. The residue was purified by reverse flash chromatography with the following conditions: column, C18 silica gel; mobile phase, TFA in Water (0.05% TFA), 0% to 50% gradient in 40 min; detector, UV 254 nm. The collected fraction was concentrated to afford 6-{2-[(tert-butyldiphenylsilyl)oxy]-1-chloroethyl}-4-(trifluoromethyl)-2,3-dihydroisoindol-1-one (4.2 g, 48.22%) as a white solid. LCMS:(ES·m/z):518,520[M+H]⁺.

### Step 8. Synthesis of Compound 9

To a stirred mixture of 6-{2-[(tert-butyldiphenylsilyl)oxy]-1-chloroethyl}-4-(trifluoromethyl)-2,3-dihydroisoindol-1-one (2 g, 3.861 mmol, 1 equiv) and (3S)-3-methylpiperidine (0.46 g, 4.63 mmol, 1.2 equiv) in DCM (60 mL) was added K₂CO₃ (2.67 g, 19.30 mmol, 5 equiv) in portions at room temperature under air atmosphere. The resulting mixture was stirred for overnight at 50° C. under air atmosphere. The mixture was allowed to cool down to room temperature. LCMS indicated the reaction was completed. The resulting mixture was diluted with water (300 mL). The resulting mixture was extracted with EtOAc (3×200 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (1:1) to afford 6-{2-[(tert-butyldiphenylsilyl)oxy]-1-[(3S)-3-methylpiperidin-1-yl]ethyl}-4-(trifluoromethyl)-2,3-dihydroisoindol-1-one (1.5 g, 66%) as a yellow oil. LCMS:(ES·m/z):581[M+H].

### Step 9. Synthesis of Compound 10

To a stirred mixture of 6-{2-[(tert-butyldiphenylsilyl)oxy]-1-[(3S)-3-methylpiperidin-1-yl]ethyl}-4-(trifluoromethyl)-2,3-dihydroisoindol-1-one (2 g, 3.44 mmol, 1 equiv) and 4-methyl-3-[(1r,3s)-1-(3-bromophenyl)-3-methylcyclobutyl]-1,2,4-triazole (1054 mg, 3.44 mmol, 1 equiv) in dioxane (20 mL) was added Cs₂CO₃ (2.24 g, 6.88 mmol, 2 equiv) and Xantphos (398 mg, 0.68 mmol, 0.2 equiv) and Pd(OAc)₂ (77 mg, 0.34 mmol, 0.1 equiv) in portions at room temperature. The resulting mixture was stirred for 2 h at 100° C. under nitrogen atmosphere. The mixture was allowed to cool down to room temperature. LCMS indicated the reaction was 60% product. The resulting mixture was concentrated under reduced pressure. The residue was purified by silica gel column chromatography, eluted with CH₂Cl₂/MeOH (10:1) to afford 6-{2-[(tert-butyldiphenylsilyl)oxy]-1-[(3S)-3-methylpiperidin-1-yl]ethyl}-2-{3-[(1r,3s)-3-methyl-1-(4-methyl-1,2,4-triazol-3-yl)cyclobutyl]phenyl}-4-(trifluoromethyl)-3H-isoindol-1-one (2.6 g, 84%) as a yellow solid. LCMS:(ES·m/z):806[M+H]⁺.

### Step 10: Synthesis of Compound 11

To a stirred solution 6-{2-[(tert-butyldiphenylsilyl)oxy]-1-[(3S)-3-methylpiperidin-1-yl]ethyl}-2-{3-[(1r,3s)-3-methyl-1-(4-methyl-1,2,4-triazol-3-yl)cyclobutyl]phenyl}-4-(trifluoromethyl)-3H-isoindol-1-one (2.5 g, 3.10 mmol, 1 equiv) in THE (40 mL) was added TBAF (1N in THF, 6 mL, 2 equiv) dropwise at 0° C. under air atmosphere. The resulting mixture was stirred for 6 h at room temperature under air atmosphere. LCMS indicated the reaction was 50% product. The resulting mixture was applied on silica gel and concentrated under reduced pressure. The residue was purified by silica gel column chromatography, eluted with CH₂Cl₂/MeOH (10:1) to afford the crude product (1 g, 80% purity). The crude product was re-purified by silica gel column chromatography, eluted with CH₂Cl₂/MeOH (10:1) to afford the 6-{2-hydroxy-1-[(3S)-3-methylpiperidin-1-yl]ethyl}-2-{3-[(1r,3s)-3-methyl-1-(4-methyl-1,2,4-triazol-3-yl)cyclobutyl]phenyl}-4-(trifluoromethyl)-3H-isoindol-1-one (667.7 mg, 37%) as a yellow solid. LCMS:(ES·m/z):568[M+H]⁺; ¹H NMR (400 MHz, DMSO-d₆) δ8.30 (s, 1H), 8.07 (s, 1H), 8.02-7.92 (m, 2H), 7.69 (d, J=7.8 Hz, 1H), 7.46 (t, J=8.0 Hz, 1H), 7.14 (d, J=7.8 Hz, 1H), 5.21 (s, 2H), 4.62 (br s, 1H), 3.83 (br s, 2H), 3.68 (br s, 1H), 3.22 (s, 3H), 3.05-2.86 (m, 3H), 2.68-2.62 (m, 1H), 2.58-2.55 (m, 3H), 1.97-1.80 (m, 1H), 1.72-1.55 (m, 4H), 1.48-1.28 (m, 1H), 1.15-1.12 (m, 3H), 0.92-1.88 (m, 1H), 1.85-1.72 (m, 3H).

### Step 11: Synthesis of Compound 151

To a stirred solution of 6-{2-hydroxy-1-[(3S)-3-methylpiperidin-1-yl]ethyl}-2-{3-[(1r,3s)-3-methyl-1-(4-methyl-1,2,4-triazol-3-yl)cyclobutyl]phenyl}-4-(trifluoromethyl)-3H-isoindol-1-one (50 mg, 0.08 mmol, 1 equiv) in ACN (1 mL) was added t-BuOK (12 mg, 0.11 mmol, 1.2 equiv) and propargyl bromide (12 mg, 0.11 mmol, 1.2 equiv) in portions at room temperature under air atmosphere. The resulting mixture was stirred for 6 h at 80° C. under air atmosphere. The mixture was allowed to cool down to room temperature. LCMS indicated the reaction was completed. The resulting mixture was concentrated under reduced pressure. The crude product (50 mg) was purified by Prep-HPLC with the following conditions (Column: XSelect CSH Prep C18 OBD Column, 19*150 mm, 5 m; Mobile Phase A: Water (0.1% FA), Mobile Phase B: ACN; Flow rate: 25 mL/min; Gradient: 10% B to 40% B in 7 min, 40% B; Wave Length: 254 nm; RT1 (min): 5.2; The collected fraction was lyophilized to afford 6-{1-[(3S)-3-methylpiperidin-1-yl]-2-(prop-2-yn-1-yloxy)ethyl}-2-{3-[(1r,3s)-3-methyl-1-(4-methyl-1,2,4-triazol-3-yl)cyclobutyl]phenyl}-4-(trifluoromethyl)-3H-isoindol-1-one (8.2 mg, 12%) as a white solid. LCMS:(ES·m/z):606[M+H-TFA]⁺; ¹H NMR (400 MHz, DMSO-d₆) δ10.07 (s, 1H), 8.30-7.94 (m, 4H), 7.85-7.78 (m, 1H), 7.54-7.44 (m, 1H), 7.21-7.18 (d, J=12 Hz, 1H), 5.36 (s, 2H), 5.25 (s, 2H), 5.16-5.07 (m, 1H), 3.94 (s, 1H), 3.88-3.84 (m, 1H), 3.70-3.67 (m, 1H), 3.45 (s, 3H), 3.10-2.80 (m, 4H), 2.76-2.60 (m, 4H), 2.35-2.15 (m, 1H), 2.07-1.85 (m, 1H), 1.68-1.43 (m, 4H), 1.23-1.11 (m, 3H), 0.92-0.62 (m, 4H)

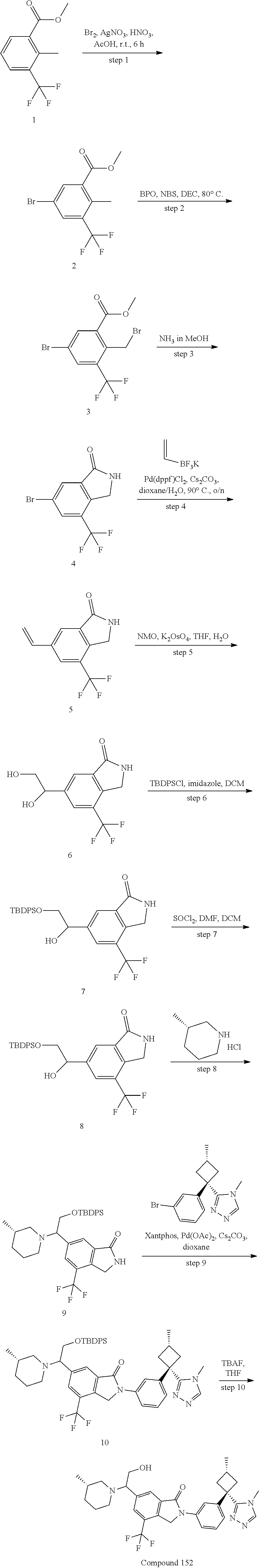

### Step 1: Synthesis of Compound 2

To a stirred solution of methyl 2-methyl-3-(trifluoromethyl)benzoate (100 g, 458.34 mmol, 1 equiv) in AcOH (650 mL) was added HNO₃ (214 mL) dropwise at 0° C. under nitrogen atmosphere. The resulting mixture was stirred for 15 min at 0° C. under nitrogen atmosphere. To the above mixture was added Br₂ (25.83 mL, 504.18 mmol, 1.1 equiv) dropwise at 0° C. The resulting mixture was stirred for additional 15 min at 0° C. To the above mixture was added AgNO₃ (101.22 g, 595.85 mmol, 1.3 equiv) in portions at 0° C. The resulting mixture was stirred for additional overnight at room temperature. GCMS and TLC indicated the reaction was completed. The resulting mixture was diluted with water (1000 mL). The resulting mixture was extracted with EtOAc (3×500 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EA (1:1) to afford methyl 5-bromo-2-methyl-3-(trifluoromethyl)benzoate (100 g, 73%) as a light yellow oil. GCMS:(ES·m/z):296,298[M]⁺.

### Step 2: Synthesis of Compound 3

To a stirred solution of methyl 5-bromo-2-methyl-3-(trifluoromethyl)benzoate (20 g, 67.32 mmol, 1.00 equiv) and NBS (17.97 g, 100.98 mmol, 1.5 equiv) in 1,2-dichloroethane (200 mL) were added BPO (3.45 g, 13.46 mmol, 0.2 equiv) at room temperature under nitrogen atmosphere. The resulting mixture was stirred for overnight at 80 degrees C. under nitrogen atmosphere. GCMS indicated complete reaction. The resulting mixture was extracted with EtOAc (3×200 mL). The combined organic layers were washed with brine (500 mL), dried over anhydrous Na₂SO₄. After filtration, the filtrate was concentrated under reduced pressure. The residue was purified by silica gel column chromatography, eluted with PE/EA (50:1) to afford methyl 5-bromo-2-(bromomethyl)-3-(trifluoromethyl)benzoate (19.7 g, 77%) as a yellow oil. ¹H NMR (300 MHz, Chloroform-d) δ 8.18 (d, J=2.1 Hz, 1H), 7.94 (d, J=2.1 Hz, 1H), 5.04 (s, 3H), 3.99 (s, 3H).

### Step 3: Synthesis of Compound 4

To a stirred solution of methyl 5-bromo-2-(bromomethyl)-3-(trifluoromethyl)benzoate (130 g, 345.77 mmol, 1 equiv) in NH₃ (7N in MeOH, 1200 mL) dropwise at 0° C. under air atmosphere. The resulting mixture was stirred for 3 h at 0° C. under nitrogen atmosphere. LCMS indicated the reaction was completed. The resulting mixture was concentrated under reduced pressure. The residue was purified by trituration with H₂O (500 mL) and PE (500 mL). This result in 6-bromo-4-(trifluoromethyl)-2,3-dihydroisoindol-1-one (70 g, 72%) as a brown solid. LCMS:(ES·m/z):280,282[M+H]⁺.

### Step 4: Synthesis of Compound 5

To a stirred mixture of 6-bromo-4-(trifluoromethyl)-2,3-dihydroisoindol-1-one (40 g, 142.83 mmol, 1 equiv) and ethenyltrifluoro-lambda4-borane potassium (22.96 g, 171.40 mmol, 1.2 equiv) in dioxane (400 mL) and H₂O (80 mL) was added Cs₂CO₃ (93.08 g, 285.67 mmol, 2 equiv) and Pd(dppf)Cl₂ (5.23 g, 7.14 mmol, 0.05 equiv) in portions at room temperature. The resulting mixture was stirred for overnight at 90° C. under nitrogen atmosphere. The mixture was allowed to cool down to room temperature. LCMS indicated the reaction was completed. The resulting mixture was filtered, the filter cake was washed with EtOAc (3×200 mL). The filtrate was concentrated under reduced pressure. The residue was purified by silica gel column chromatography, eluted with CH₂Cl₂/MeOH (10:1) to afford 6-ethenyl-4-(trifluoromethyl)-2,3-dihydroisoindol-1-one (28 g, 86%) as a yellow solid. LCMS:(ES·m/z):228[M+H]⁺.

### Step 5: Synthesis of Compound 6

To a stirred solution of 6-ethenyl-4-(trifluoromethyl)-2,3-dihydroisoindol-1-one (2.8 g, 12.32 mmol, 1 equiv) in THE (30 mL) and H₂O (15 mL) was added NMO (4.33 g, 36.97 mmol, 3 equiv) and Potassium osmate(VI) dihydrate (0.23 g, 0.61 mmol, 0.05 equiv) in portions at room temperature under air atmosphere. The resulting mixture was stirred for 2 h at room temperature under air atmosphere. LCMS indicated the reaction was completed. The resulting mixture was concentrated under reduced pressure. The residue was purified by reverse flash chromatography with the following conditions: column, C18 silica gel; mobile phase, TFA in Water (0.05% TFA), 0% to 50% gradient in 40 min; detector, UV 254 nm. The collected fraction was concentrated to afford 6-(1,2-dihydroxyethyl)-4-(trifluoromethyl)-2,3-dihydroisoindol-1-one (2.1 g, 65%) as a yellow solid. LCMS:(ES·m/z):262[M+H]⁺.

### Step 6: Synthesis of Compound 7

To a stirred solution of 6-(1,2-dihydroxyethyl)-4-(trifluoromethyl)-2,3-dihydroisoindol-1-one (9.7 g, 37.13 mmol, 1 equiv) in DCM (200 mL) was added imidazole (5.06 g, 74.27 mmol, 2 equiv) in portions and TBDPSCl (10.21 g, 37.13 mmol, 1 equiv) dropwise at room temperature under air atmosphere. The resulting mixture was stirred for 2 h at room temperature under air atmosphere. LCMS indicated the reaction was completed. The resulting mixture was diluted with water (30 mL). The resulting mixture was extracted with CH₂Cl₂ (3×40 mL). The combined organic layers were washed with brine (2×10 mL), dried over anhydrous Na₂SO₄. After filtration, the filtrate was concentrated under reduced pressure. The residue was purified by silica gel column chromatography, eluted with CH₂Cl₂/MeOH (10:1) to afford 6-{2-[(tert-butyldiphenylsilyl)oxy]-1-hydroxyethyl}-4-(trifluoromethyl)-2,3-dihydroisoindol-1-one (8.3 g, 44%) as a pink solid. LCMS:(ES·m/z):500[M+H].

### Step 7: Synthesis of Compound 8

To a stirred solution of 6-{2-[(tert-butyldiphenylsilyl)oxy]-1-hydroxyethyl}-4-(trifluoromethyl)-2,3-dihydroisoindol-1-one (8.4 g, 16.81 mmol, 1 equiv) in DCM (120 mL) was added SOCl₂ (6.00 g, 50.43 mmol, 3 equiv) dropwise at 0° C. under air atmosphere. The resulting mixture was stirred for 2 h at 0° C. under air atmosphere. LCMS indicated the reaction was 50% product. The resulting mixture was concentrated under reduced pressure. The residue was purified by reverse flash chromatography with the following conditions: column, C18 silica gel; mobile phase, TFA in Water (0.05% TFA), 0% to 50% gradient in 40 min; detector, UV 254 nm. The collected fraction was concentrated to afford 6-{2-[(tert-butyldiphenylsilyl)oxy]-1-chloroethyl}-4-(trifluoromethyl)-2,3-dihydroisoindol-1-one (4.2 g, 48.22%) as a white solid. LCMS:(ES·m/z):518,520[M+H]⁺.

### Step 8: Synthesis of Compound 9

To a stirred mixture of 6-{2-[(tert-butyldiphenylsilyl)oxy]-1-chloroethyl}-4-(trifluoromethyl)-2,3-dihydroisoindol-1-one (2 g, 3.861 mmol, 1 equiv) and (3S)-3-methylpiperidine (0.46 g, 4.63 mmol, 1.2 equiv) in DCM (60 mL) was added K₂CO₃ (2.67 g, 19.30 mmol, 5 equiv) in portions at room temperature under air atmosphere. The resulting mixture was stirred for overnight at 50° C. under air atmosphere. The mixture was allowed to cool down to room temperature. LCMS indicated the reaction was completed. The resulting mixture was diluted with water (300 mL). The resulting mixture was extracted with EtOAc (3×200 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (1:1) to afford 6-{2-[(tert-butyldiphenylsilyl)oxy]-1-[(3S)-3-methylpiperidin-1-yl]ethyl}-4-(trifluoromethyl)-2,3-dihydroisoindol-1-one (1.5 g, 66%) as a yellow oil. LCMS:(ES·m/z):581[M+H]⁺.

### Step 9: Synthesis of Compound 10

To a stirred mixture of 6-{2-[(tert-butyldiphenylsilyl)oxy]-1-[(3S)-3-methylpiperidin-1-yl]ethyl}-4-(trifluoromethyl)-2,3-dihydroisoindol-1-one (2 g, 3.44 mmol, 1 equiv) and 4-methyl-3-[(1r,3s)-1-(3-bromophenyl)-3-methylcyclobutyl]-1,2,4-triazole (1054 mg, 3.44 mmol, 1 equiv) in dioxane (20 mL) was added Cs₂CO₃ (2.24 g, 6.88 mmol, 2 equiv) and Xantphos (398 mg, 0.68 mmol, 0.2 equiv) and Pd(OAc)₂ (77 mg, 0.34 mmol, 0.1 equiv) in portions at room temperature. The resulting mixture was stirred for 2 h at 100° C. under nitrogen atmosphere. The mixture was allowed to cool down to room temperature. LCMS indicated the reaction was 60% product. The resulting mixture was concentrated under reduced pressure. The residue was purified by silica gel column chromatography, eluted with CH₂Cl₂/MeOH (10:1) to afford 6-{2-[(tert-butyldiphenylsilyl)oxy]-1-[(3S)-3-methylpiperidin-1-yl]ethyl}-2-{3-[(1r,3s)-3-methyl-1-(4-methyl-1,2,4-triazol-3-yl)cyclobutyl]phenyl}-4-(trifluoromethyl)-3H-isoindol-1-one (2.6 g, 84%) as a yellow solid. LCMS:(ES·m/z):806[M+H]⁺.

### Step 10: Synthesis of Compound 152

To a stirred solution 6-{2-[(tert-butyldiphenylsilyl)oxy]-1-[(3S)-3-methylpiperidin-1-yl]ethyl}-2-{3-[(1r,3s)-3-methyl-1-(4-methyl-1,2,4-triazol-3-yl)cyclobutyl]phenyl}-4-(trifluoromethyl)-3H-isoindol-1-one (2.5 g, 3.10 mmol, 1 equiv) in THE (40 mL) was added TBAF (1N in THF, 6 mL, 2 equiv) dropwise at 0° C. under air atmosphere. The resulting mixture was stirred for 6 h at room temperature under air atmosphere. LCMS indicated the reaction was 50% product. The resulting mixture was applied on silica gel and concentrated under reduced pressure. The residue was purified by silica gel column chromatography, eluted with CH₂Cl₂/MeOH (10:1) to afford the crude product (1 g, 80% purity). The crude product was re-purified by silica gel column chromatography, eluted with CH₂Cl₂/MeOH (10:1) to afford the 6-{2-hydroxy-1-[(3S)-3-methylpiperidin-1-yl]ethyl}-2-{3-[(1r,3s)-3-methyl-1-(4-methyl-1,2,4-triazol-3-yl)cyclobutyl]phenyl}-4-(trifluoromethyl)-3H-isoindol-1-one (667.7 mg, 37%) as a yellow solid. LCMS:(ES·m/z):568[M+H]⁺; ¹H NMR (400 MHz, DMSO-d₆) δ8.30 (s, 1H), 8.07 (s, 1H), 8.02-7.92 (m, 2H), 7.69 (d, J=7.8 Hz, 1H), 7.46 (t, J=8.0 Hz, 1H), 7.14 (d, J=7.8 Hz, 1H), 5.21 (s, 2H), 4.62 (br s, 1H), 3.83 (br s, 2H), 3.68 (br s, 1H), 3.22 (s, 3H), 3.05-2.86 (m, 3H), 2.68-2.62 (m, 1H), 2.58-2.55 (m, 3H), 1.97-1.80 (m, 1H), 1.72-1.55 (m, 4H), 1.48-1.28 (m, 1H), 1.15-1.12 (m, 3H), 0.92-1.88 (m, 1H), 1.85-1.72 (m, 3H).

### Synthesis of Compound 153

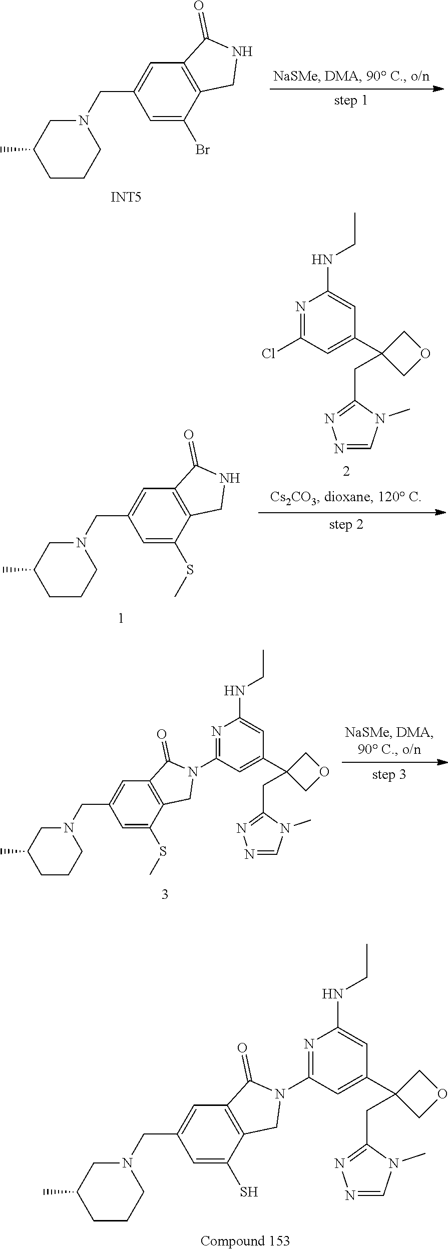

### Step 1: Synthesis of Compound 1

To a stirred solution of 4-bromo-6-{[(3S)-3-methylpiperidin-1l-yl]methyl}-2,3-dihydroisoindol-1-one (INT5, 300 mg, 0.92 mmol, 1 equiv) in DMA (3 mL) was added sodium thiomethoxide (97 mg, 1.39 mmol, 1.5 equiv) at room temperature under nitrogen atmosphere. The resulting mixture was stirred for 1 h at 90° C. under nitrogen atmosphere. LCMS indicated the reaction was completed. The mixture was allowed to cool down to room temperature. The residue was purified by reverse flash chromatography with the following conditions: column, C18 silica gel; mobile phase, MeCN in Water (0.05% TFA), 5% to 50% gradient in 30 min; detector, UV 254 nm. The collected fraction was concentrated to afford 6-{[(3S)-3-methylpiperidin-1l-yl]methyl}-4-(methylsulfanyl)-2,3-dihydroisoindol-1-one (210 mg, 77%) as an off-white solid. LCMS (ESI, m/z):291 [M+H]⁺

### Step 2: Synthesis of Compound 3

To a stirred solution of 6-{[(3S)-3-methylpiperidin-1l-yl]methyl}-4-(methylsulfanyl)-2,3-dihydroisoindol-1-one (210 mg, 0.72 mmol, 1 equiv) and 6-chloro-N-ethyl-4-{3-[(4-methyl-1,2,4-triazol-3-yl)methyl]oxetan-3-yl}pyridin-2-amine (222 mg, 0.72 mmol, 1 equiv) in dioxane (6 mL) was added Cs₂CO₃ (471 mg, 1.44 mmol, 2 equiv) Xantphos (83 mg, 0.145 mmol, 0.2 equiv) and Pd(OAC)₂ (16.23 mg, 0.072 mmol, 0.1 equiv) at room temperature under nitrogen atmosphere. The resulting mixture was stirred for 2 h at 120° C. under nitrogen atmosphere. LCMS indicated the reaction was completed. The mixture was allowed to cool down to room temperature. The residue was purified by reverse flash chromatography with the following conditions: column, C18 silica gel; mobile phase, MeCN in Water (0.1% TFA), 5% to 60% gradient in 10 min; detector, UV 254 nm. This resulted in 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (110 mg, 27%) as a yellow solid. LCMS (ESI, m/z):562[M+H]⁺

### Step 3: Synthesis of Compound 153

To a stirred solution of 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (110 mg, 0.19 mmol, 1 equiv) in DMA (2 mL) was added Sodium thiomethoxide (68 mg, 0.98 mmol, 5 equiv) in portions at room temperature under nitrogen atmosphere. The resulting mixture was stirred for 3 h at 90° C. under nitrogen atmosphere. LCMS indicated the reaction was completed. The mixture was allowed to cool down to room temperature. The residue was purified by reverse flash chromatography with the following conditions: column, C18 silica gel; mobile phase, MeCN in Water (0.05% TFA), 5% to 60% gradient in 30 min; detector, UV 254 nm. The crude product was purified by Prep-HPLC with the following conditions (Column: XBridge Prep OBD C18 Column, 19*250 mm, 5 m; Mobile Phase A: Water (0.1% TFA), Mobile Phase B: ACN; Flow rate: 25 mL/min; Gradient: 22% B to 48% B in 10 min, 48% B; Wave Length: 254 nm; RT1 (min): 7.05). The collected fraction was lyophilized to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-sulfanyl-3H-isoindol-1-one (2.3 mg) as a yellow solid. LCMS (ESI, m/z):548[M+H−TFA]⁺; ¹H NMR (300 MHz, CD₃OD) δ 8.56-8.50 (m, 1H), 8.01 (s, 1H), 7.90 (s, 1H), 7.23 (s, 1H), 5.94 (s, 1H), 5.11-4.96 (m, 6H), 4.44 (br s, 2H), 3.70 (s, 2H), 3.57-3.36 (m, 4H), 3.23-3.07 (m, 2H), 2.86-2.80 (m, 1H), 2.62-2.58 (m, 1H), 1.95-1.67 (m, 5H), 1.28-1.18 (m, 4H), 0.93-0.91 (m, 3H).

### Synthesis of Compound 154

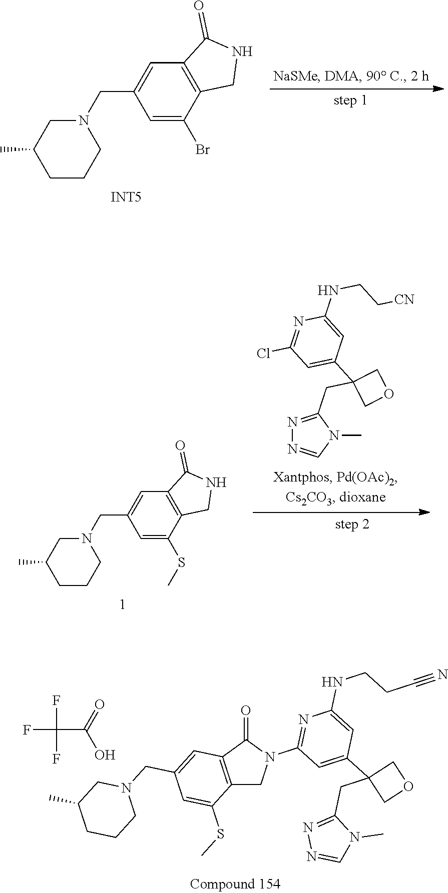

### Step 1: Synthesis of Compound 1

To a stirred solution of 4-bromo-6-{[(3S)-3-methylpiperidin-1-yl]methyl}-2,3-dihydroisoindol-1-one (INT5, 500 mg, 1.54 mmol, 1 equiv) in DMF (5 mL) was added sodium methanethiolate (162 mg, 2.32 mmol, 1.5 equiv) in portions at room temperature under nitrogen atmosphere. The resulting mixture was stirred for 1 h at 90° C. under nitrogen atmosphere. LCMS indicated the reaction was completed. The mixture was allowed to cool down to room temperature. The residue was purified by reverse flash chromatography with the following conditions: column, C18 silica gel; mobile phase, MeCN in Water (0.05% TFA), 5% to 60% gradient in 30 min; detector, UV 254 nm. The collected fraction was concentrated to afford 6-{[(3S)-3-methylpiperidin-1-yl]methyl}-4-(methylsulfanyl)-2,3-dihydroisoindol-1-one (410 mg, 91%) as a white solid. LCMS (ESI, m/z):291[M+H]⁺

### Step 2: Synthesis of Compound 154

To a stirred solution of 3-[(6-chloro-4-{3-[(4-methyl-1,2,4-triazol-3-yl)methyl]oxetan-3-yl}pyridin-2-yl)amino]propanenitrile (50 mg, 0.15 mmol, 1 equiv) and 6-{[(3S)-3-methylpiperidin-1-yl]methyl}-4-(methylsulfanyl)-2,3-dihydroisoindol-1-one (50 mg, 0.16 mmol, 1.1 equiv) in dioxane (1 mL) was added Cs₂CO₃ (98 mg, 0.30 mmol, 2 equiv), XantPhos (15 mg, 0.03 mmol, 0.2 equiv) and Pd(OAc)₂ (4 mg, 0.015 mmol, 0.1 equiv) at room temperature under nitrogen atmosphere. The resulting mixture was stirred for 3 h at 120° C. under nitrogen atmosphere. LCMS indicated the reaction was completed. The mixture was allowed to cool down to room temperature. The reaction mixture was purified by reverse flash chromatography with the following conditions: column, C18 silica gel; mobile phase, MeCN in Water (0.05% TFA), 5% to 60% gradient in 30 min; detector, UV 254 nm. The collected fraction was concentrated to afford crude product as an off-white solid. The crude product (20 mg) was purified by Prep-HPLC with the following conditions (Column: XBridge Shield RP18 OBD Column, 19*250 mm, 10 m; Mobile Phase A: Water (0.1% TFA), Mobile Phase B: ACN; Flow rate: 25 mL/min; Gradient: 19% B to 49% B in 8 min, 49% B; Wave Length: 254 nm; RT1 (min): 5.7; The collected fraction was lyophilized to afford 3-[(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)amino]propanenitrile; trifluoroacetic acid (5.1 mg, 5%) as a white solid. LCMS (ESI, m/z):587[M+H−TFA]⁺; ¹H NMR (300 MHz, CD₃OD) δ 8.61 (s, 1H), 7.64 (s, 1H), 7.59 (s, 1H), 7.41 (s, 1H), 5.97 (s, 1H), 5.00-4.87 (m, 4H), 4.85 (s, 2H), 4.43 (s, 2H), 3.71-3.67 (m, 4H), 3.49-3.36 (m, 2H), 3.29 (s, 3H), 2.96-2.92 (m, 1H), 2.88-2.81 (m, 2H), 2.79-2.70 (m, 1H), 2.50 (s, 3H), 1.98-1.70 (m, 4H), 1.28-1.10 (m, 1H), 0.89 (d, J=6.6 Hz, 3H).

### Synthesis of Compound 155

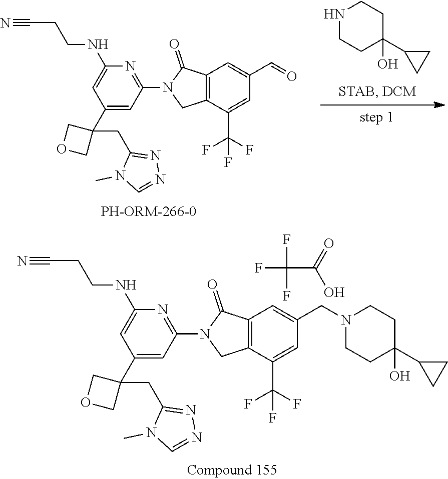

### Step 1: Synthesis of Compound 155

To a stirred solution of 3-({6-[6-formyl-1-oxo-4-(trifluoromethyl)-3H-isoindol-2-yl]-4-{3-[(4-methyl-1,2,4-triazol-3-yl)methyl]oxetan-3-yl}pyridin-2-yl}amino)propanenitrile (Compound 160-A, 100 mg, 0.19 mmol, 1 equiv) and 4-cyclopropylpiperidin-4-ol (32 mg, 0.23 mmol, 1.19 equiv) in DCM was added sodium bis(acetyloxy)boranuidyl acetate (161 mg, 0.76 mmol, 3.99 equiv) in portions at room temperature under air atmosphere. The mixture was stirred for 2 h at room temperature. LCMS indicated the reaction was completed. The reaction was quenched by the addition of MeOH (3 mL) at room temperature. The resulting mixture was concentrated under vacuum. The residue was purified by reverse flash chromatography with the following conditions: column, C18 silica gel; mobile phase, MeCN in Water (0.05% TFA), 5% to 60% gradient in 30 min; detector, UV 254 nm. The eluted was concentrated under vacuum to dryness. The crude product was purified by Prep-HPLC with the following conditions (Column: XBridge Prep OBD C18 Column, 19*250 mm, 5 m; Mobile Phase A: Water (0.1% TFA), Mobile Phase B: ACN; Flow rate: 25 mL/min; Gradient: 20% B to 42% B in 10 min, 42% B; Wave Length: 254 nm; RT1 (min): 6.95; The collected fraction was lyophilized to afford 3-[(6-{6-[(4-cyclopropyl-4-hydroxypiperidin-1-yl)methyl]-1-oxo-4-(trifluoromethyl)-3H-isoindol-2-yl}-4-{3-[(4-methyl-1,2,4-triazol-3-yl)methyl]oxetan-3-yl}pyridin-2-yl)amino]propanenitrile (9.1 mg, 7.17%) as a light yellow solid. LCMS (MS, E/S): 651 [M+H−TFA]; H-NMR (300 MHz, CD₃OD): 8.70 (br s, 1H), 8.28 (s, 1H), 8.17 (s, 1H), 7.36 (s, 1H), 5.99 (s, 1H), 5.35 (s, 2H), 5.02-4.97 (m, 4H), 4.56 (s, 2H), 3.96-3.65 (m, 4H), 3.38-3.30 (m, 7H), 2.86-2.78 (m, 2H), 2.13-1.76 (m, 4H), 0.89-0.88 (m, 1H), 0.39-0.29 (m, 4H).

### Synthesis of Compound 156

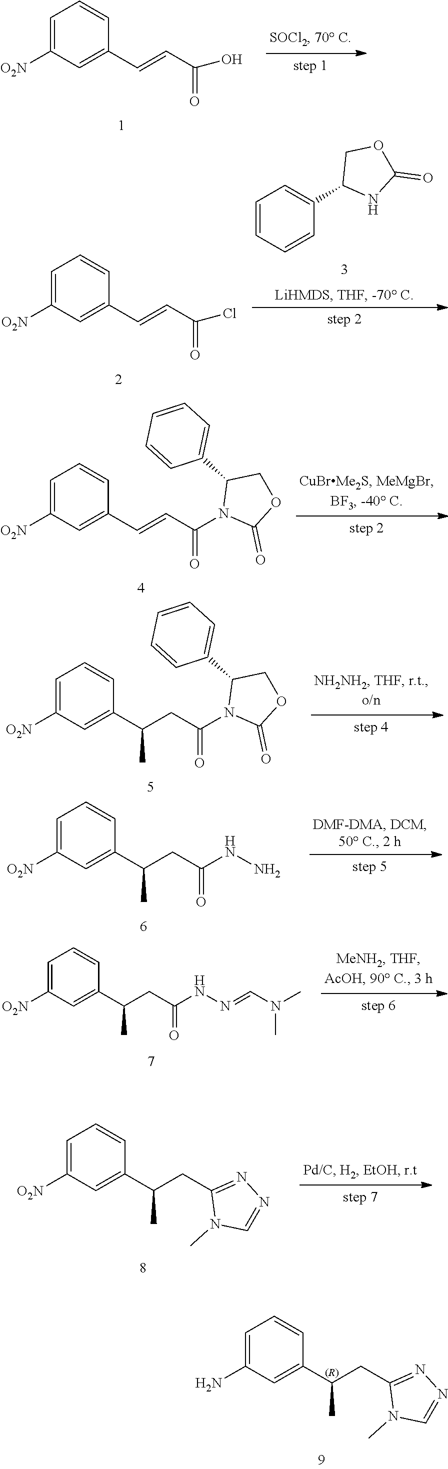

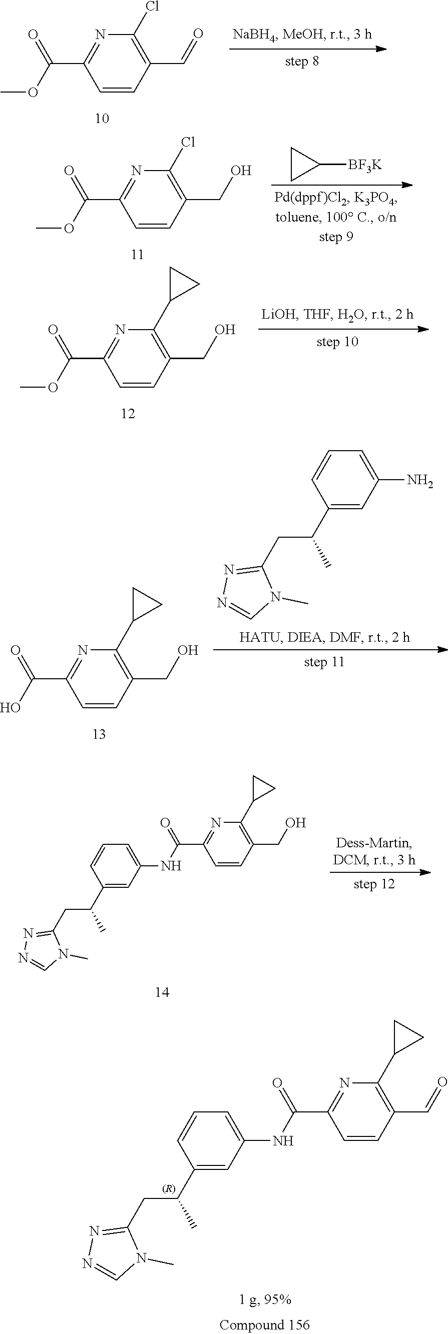

### Step 1: Synthesis of Compound 2

To a stirred solution of 3-nitrocinnamic acid (50 g, 258.86 mmol, 1.00 equiv) in SOCl₂ (250 mL) at room temperature under nitrogen atmosphere. The resulting mixture was stirred for 2 h at 80° C. under nitrogen atmosphere. Desired product could be detected by LCMS (added MeOH). LCMS indicated the reaction was completed. The resulting mixture was concentrated under vacuum. The crude product was further confirmed by H-NMR (added MeOH). ¹H NMR (300 MHz, CDCl₃, derivative with MeOH) δ 8.39 (t, J=3 Hz, 1H), 8.27-8.23 (m, 1H), 7.86-7.83 (m, 1H), 7.74 (d, J=6 Hz, 1H), 7.61 (t, J=6 Hz, 1H), 6.58 (d, J=6 Hz, 1H), 3.85 (s, 3H).

### Step 2: Synthesis of Compound 4

To a stirred mixture of (4R)-4-phenyl-1,3-oxazolidin-2-one (23 g, 141.75 mmol, 1.00 equiv) in THE (300 mL) were added LiHMDS (135 mL) at −70° C. under nitrogen atmosphere. The mixture of reaction was stirred for 1 h at −70° C. under nitrogen atmosphere. To the above mixture was added (2E)-3-(3-nitrophenyl)prop-2-enoyl chloride (30 g, 141.78 mmol, 1.00 equiv)(THF=300.00 mL)). The resulting mixture was stirred for overnight at room temperature under nitrogen atmosphere. Desired product could be detected by LCMS and TLC. LCMS indicated the reaction was completed. The reaction was quenched with NH₄Cl. The aqueous layer was extracted with DCM. The organic layer was concentrated under vacuum to afford (4R)-3-[(2E)-3-(3-nitrophenyl)prop-2-enoyl]-4-phenyl-1,3-oxazolidin-2-one (32 g, 50%) as a yellow solid. LCMS (ES, m/z):339 [M+H]⁺.

### Step 3: Synthesis of Compound 5

To a stirred solution of Copper(I) bromide-dimethyl sulfide (29 g, 141.90 mmol, 1.50 equiv) in THE (160 mL) was added MeMgBr (94 mL, 3M in THF, 283.77 mmol, 3.00 equiv) in portions at −40° C. under nitrogen atmosphere. The resulting mixture was stirred for 1 h min at −30° C.-20° C. under nitrogen atmosphere. To the above mixture was added BF₃·Et₂O (20 g, 141.90 mmol, 1.50 equiv) in portions at −40° C. under nitrogen atmosphere. The resulting mixture was stirred for additional 1 h at −30° C.˜-20° C. under nitrogen atmosphere. Finally, to the above mixture was added (4R)-3-[(2E)-3-(3-nitrophenyl)prop-2-enoyl]-4-phenyl-1,3-oxazolidin-2-one (32 g, 94.58 mmol, 1.00 equiv)(THF (160 mL) in portions at −40° C. under nitrogen atmosphere. The resulting mixture was stirred for 2 h at −30° C.˜-20° C. under nitrogen atmosphere. Desired product could be detected by LCMS. LCMS indicated the reaction was completed. The reaction was quenched with sat. NH₄Cl (aq.) at room temperature. The resulting mixture was extraction with EA (150 mL). The resulting mixture was concentrated under vacuum. The residue was purified by silica gel column chromatography, eluted with PE/EtOAc (4:1) to afford (4R)-3-[(3R)-3-(3-nitrophenyl)butanoyl]-4-phenyl-1,3-oxazolidin-2-one (18 g, 51%) as a solid. LCMS (ES, m/z): 355 [M+H]⁺, 377 [M+Na]⁺. ¹H NMR (300 MHz, CDCl₃) δ 8.09-8.02 (m, 2H), 7.63-7.54 (m, 1H), 7.46-7.28 (m, 4H), 7.27-7.12 (m, 2H), 5.37-5.33 (m, 1H), 4.66 (t, J=6 Hz, 1H), 4.31-4.27 (m, 1H), 3.52-3.12 (m, 3H), 1.31 (t, J=9 Hz, 3H).

### Step 4: Synthesis of Compound 6

To a stirred mixture of (4R)-3-[(3R)-3-(3-nitrophenyl)butanoyl]-4-phenyl-1,3-oxazolidin-2-one (8 g, 22.58 mmol, 1.00 equiv) in THE (80 mL) were added NH₂NH₂·H₂O (2.8 g, 45.22 mmol, 2.00 equiv, 80%) in portions at room temperature under nitrogen atmosphere. The resulting mixture was stirred for overnight at room temperature under nitrogen atmosphere. Desired product could be detected by LCMS. The resulting mixture was concentrated under vacuum to afford (3R)-3-(3-nitrophenyl)butanehydrazide (9.0 g, crude). The crude product was used in the next step directly without further purification. LCMS (ES, m/z): 224 [M+H]⁺.

### Step 5: Synthesis of Compound 7

To a stirred mixture of (3R)-3-(3-nitrophenyl)butanehydrazide (5 g, 22.40 mmol, 1.00 equiv) in DCM (80 mL) were added DMF-DMA (5.30 g, 44.80 mmol, 2.00 equiv) in portions at room temperature under nitrogen atmosphere. The resulting mixture was stirred for 2 h at 50° C. under nitrogen atmosphere. Desired product could be detected by LCMS. The resulting mixture was concentrated under vacuum. The crude product was used further purification. The residue was purified by silica gel column chromatography, eluted with CH₂Cl₂/MeOH (10:1) to afford (3R)-N′-[(1E)-(dimethylamino)methylidene]-3-(3-nitrophenyl)butanehydrazide (6.4 g, 92%) as a semi-solid. LCMS (ES, m/z): 279 [M+H]⁺.

### Step 6: Synthesis of Compound 8

To a stirred mixture of (3R)-N′-[(1E)-(dimethylamino)methylidene]-3-(3-nitrophenyl)butanehydrazide (6.0 g, 21.56 mmol, 1.00 equiv) in AcOH (60 mL) were added CH₃NH₂ (1N in THF, 120 mL) in portions at room temperature under nitrogen atmosphere. The resulting mixture was stirred for 3 h at 90° C. under nitrogen atmosphere. Desired product could be detected by LCMS. The reaction was quenched with Water/Ice at room temperature. The mixture neutralized to pH=8 with saturated NaHCO₃ (aq.). The resulting mixture was extracted with CH₂Cl₂ and EA (3×30 mL). The mixture was concentrated under reduced pressure to afford 4-methyl-3-[(2R)-2-(3-nitrophenyl)propyl]-1,2,4-triazole (5.7 g, 54%) as a solid. LCMS (ES, m/z): 247 [M+H]⁺.

### Step 7: Synthesis of Compound 9

To a solution of 4-methyl-3-[(2R)-2-(3-nitrophenyl)propyl]-1,2,4-triazole (2.8 g, 11.37 mmol, 1 equiv) in 50 mL EtOH was added Pd/C (10%, 0.6 g) under nitrogen atmosphere. The mixture was hydrogenated at room temperature for overnight under hydrogen atmosphere using a hydrogen balloon, LCMS indicated the reaction was completed. The mixture was filtered through a Celite pad and concentrated under reduced pressure. This resulted in 3-[(2R)-1-(4-methyl-1,2,4-triazol-3-yl)propan-2-yl]aniline (2.4 g, 97%) as a yellow oil. LCMS (ES, m/z):217[M+H]⁺

### Step 8: Synthesis of Compound 11

To a stirred solution of methyl 6-chloro-5-formylpyridine-2-carboxylate (5.0 g, 25.05 mmol, 1.00 equiv) in MeOH (50 mL) were added NaBH₄ (0.57 g, 15.03 mmol, 0.6 equiv) in portions at 0° C. under nitrogen atmosphere. The resulting mixture was stirred for 2 h at room temperature under nitrogen atmosphere. Desired product could be detected by LCMS. The reaction was quenched with Water at room temperature. The aqueous layer was extracted with CH₂Cl₂ (3×100 mL). The resulting mixture was concentrated under vacuum. The residue was purified by silica gel column chromatography, eluted with PE/EA (1:1) to afford methyl 6-chloro-5-(hydroxymethyl)pyridine-2-carboxylate (3.5 g, 69%) as a white solid. LCMS: (ES, m/s): 202 [M+H], 224 [M+Na]⁺.

### Step 9: Synthesis of Compound 12

To a stirred solution of methyl 6-chloro-5-(hydroxymethyl)pyridine-2-carboxylate (3.5 g, 17.36 mmol, 1.00 equiv) in Toluene (35.00 mL) and H2O (14.00 mL) were added cyclopropyltrifluoro-lambda4-borane potassium (7.71 g, 52.08 mmol, 3.0 equiv), K3PO₄ (11.05 g, 52.08 mmol, 3.0 equiv) and Pd(dppf)Cl₂ (2.54 g, 3.47 mmol, 0.2 equiv) in portions under nitrogen atmosphere. The resulting mixture was stirred for overnight at 100° C. under nitrogen atmosphere. Desired product could be detected by LCMS. The resulting mixture was concentrated under vacuum. The residue was purified by silica gel column chromatography, eluted with PE/EA (1:1) to afford methyl 6-cyclopropyl-5-(hydroxymethyl)pyridine-2-carboxylate (3.0 g, 80%) as a light yellow oil. LCMS: (ES, m/s): 208 [M+H]⁺, 230 [M+Na]⁺.

### Step 10: Synthesis of Compound 13

To a stirred solution of methyl 6-cyclopropyl-5-(hydroxymethyl)pyridine-2-carboxylate (3.0 g, 14.47 mmol, 1.00 equiv) in THF (30.00 mL) were added LiGH (0.69 g, 28.95 mmol, 2.0 equiv)(H₂O=12.00 mL) in portions at room temperature under nitrogen atmosphere. The resulting mixture was stirred for 3 h at room temperature under nitrogen atmosphere. LCMS indicated the reaction was completed. The mixture neutralized to pH 6-7 with conc. HCl. The resulting mixture was concentrated under vacuum. The residue was purified by reverse flash chromatography with the following conditions: column, C18 silica gel; mobile phase FA, ACN in water, 0% to 5% gradient in 20 min; detector, UV 254 nm. The resulting mixture was concentrated under vacuum to afford 6-cyclopropyl-5-(hydroxymethyl)pyridine-2-carboxylic acid (2.0 g, 67%) as a white solid. LCMS: (ES, m/s): 194 [M+H]⁺, 216 [M+Na]⁺.

### Step 11: Synthesis of Compound 14

To a stirred solution of 6-cyclopropyl-5-(hydroxymethyl)pyridine-2-carboxylic acid (2.1 g, 10.86 mmol, 1 equiv) and HATU (6.20 g, 16.30 mmol, 1.5 equiv) in DMF (21 mL) was stirred for 1 h at room temperature under nitrogen atmosphere. To the above mixture was added 3-[(2R)-1-(4-methyl-1,2,4-triazol-3-yl)propan-2-yl]aniline (2.35 g, 10.86 mmol, 1 equiv) and DIEA (2.81 g, 21.73 mmol, 2 equiv) dropwise at 0° C. The resulting mixture was stirred for additional overnight at room temperature. LCMS indicated the reaction was completed. The resulting mixture was extracted with CH₂Cl₂:MeOH (10:1) (3×100 mL). The combined organic layers were washed with water (3×100 mL), dried over anhydrous Na₂SO₄. After filtration, the filtrate was concentrated under reduced pressure. The residue was purified by silica gel column chromatography, eluted with CH₂Cl₂/MeOH (10:1) to afford 6-cyclopropyl-5-(hydroxymethyl)-N-{3-[(2R)-1-(4-methyl-1,2,4-triazol-3-yl)propan-2-yl]phenyl}pyridine-2-carboxamide (2.1 g, 49%) as a yellow oil. LCMS (ESI, m/z):392[M+H]⁺

### Step 12: Synthesis of Compound 156

To a stirred solution of 6-cyclopropyl-5-(hydroxymethyl)-N-{3-[(2R)-1-(4-methyl-1,2,4-triazol-3-yl)propan-2-yl]phenyl}pyridine-2-carboxamide (2.1 g, 5.36 mmol, 1 equiv) in DCM (22 mL) was added Dess-Martin (4.55 g, 10.72 mmol, 2 equiv) at room temperature under nitrogen atmosphere. LCMS indicated the reaction was completed. The reaction was quenched with MeOH at 0° C. The resulting mixture was concentrated under vacuum. The residue was purified by silica gel column chromatography, eluted with CH₂Cl₂/MeOH (10:1) to afford 6-cyclopropyl-5-formyl-N-{3-[(2R)-1-(4-methyl-1,2,4-triazol-3-yl)propan-2-yl]phenyl}pyridine-2-carboxamide (1.1 g, 47%) as a yellow solid. LCMS (ESI, m/z):390[M+H]⁺; ¹H NMR (300 MHz, DMSO-d₆) δ 10.50 (s, 1H), 10.15 (s, 1H), 8.37 (d, J=7.8 Hz, 1H), 8.28 (s, 1H), 8.04 (d, J=7.8 Hz, 1H), 7.73-7.66 (,. 2J). 7.33-7.28 (m, 1H), 7.07-7.05 (m, 1H), 3.46 (s, 3H), 3.31-3.28 (m, 1H), 3.26-3.21 (m, 1H), 3.00 (d, J=7.5 Hz, 2H), 1.48-1.46 (m, 2H), 1.43-1.28 (m, 3H), 1.16-1.14 (m, 2H)

### Synthesis of Compound 157

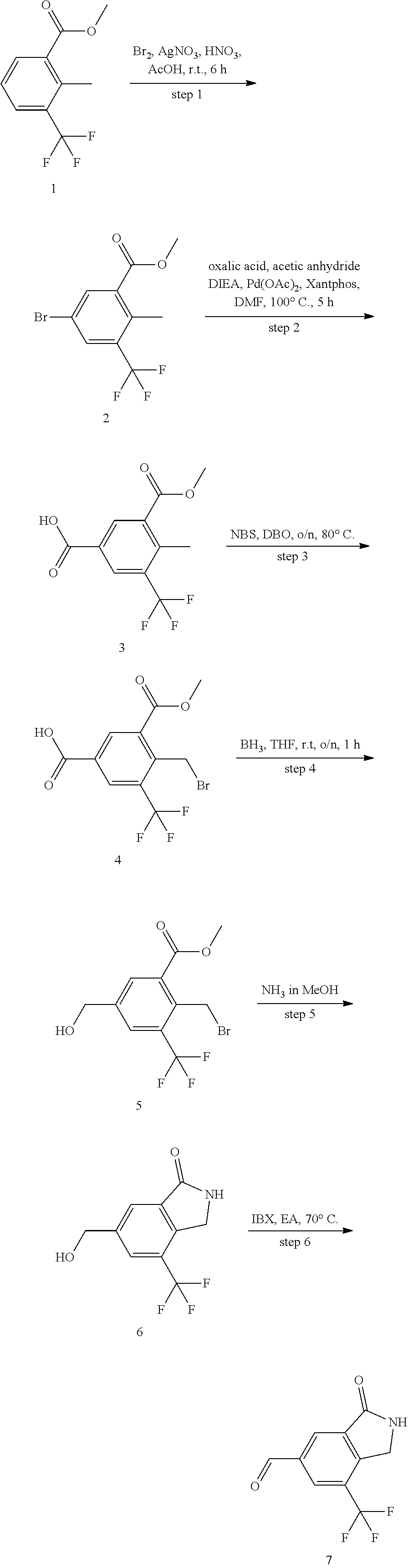

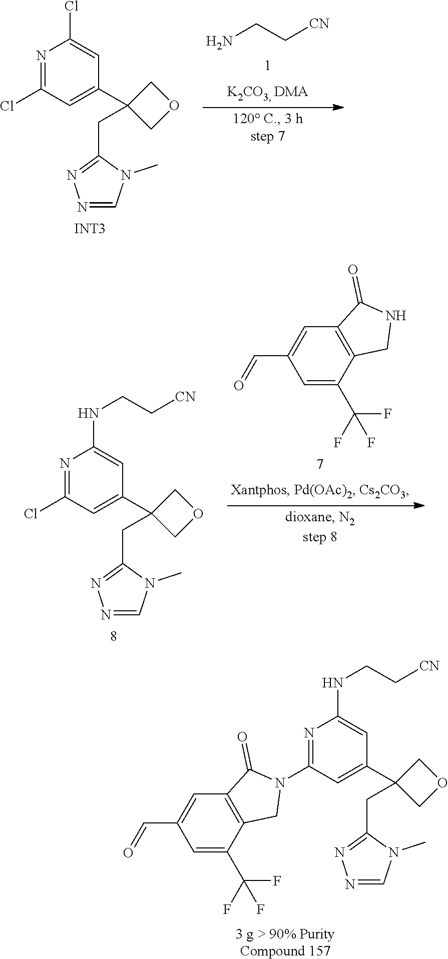

### Step 1: Synthesis of Compound 2

To a stirred mixture of methyl 2-methyl-3-(trifluoromethyl)benzoate (44 g, 201.67 mmol, 1 equiv) in AcOH (286 mL) was added HNO₃ (127 g, 2015.46 mmol, 9.99 equiv) dropwise over 20 min at 10° C. under nitrogen atmosphere. To the above mixture was added Br₂ (35.5 g, 222.14 mmol, 1.10 equiv) dropwise over 10 min at 10° C. The resulting mixture was stirred for additional 10 min at 10° C. To the above mixture was added AgNO₃ (44.4 g, 261.37 mmol, 1.30 equiv) in water (105 mL) (2.5 mol/L) dropwise over 25 min at 10° C. The resulting mixture was stirred for additional overnight at room temperature. TLC indicated the reaction was completed. The mixture was basified to pH 8 with saturated Na₂CO₃ (aq.). The resulting mixture was extracted with EtOAc (3×800 mL). The combined organic layers were washed with water (3×100 mL), dried over anhydrous Na₂SO₄. After filtration, the filtrate was concentrated under reduced pressure. The residue was purified by silica gel column chromatography, eluted with PE/EA (10:1) to afford methyl 5-bromo-2-methyl-3-(trifluoromethyl)benzoate (42 g, 70%) as a yellow oil. ¹H NMR (300 MHz, DMSO) δ 8.32 (s, 1H), 8.08 (s, 1H), 3.87 (s, 3H), 3.46-3.44 (m, 3H).

### Step 2: Synthesis of Compound 3

To a stirred mixture of methyl 5-bromo-2-methyl-3-(trifluoromethyl)benzoate (42 g, 141.38 mmol, 1.00 equiv) and acetic anhydride (21.7 g, 212.56 mmol, 1.50 equiv) in DMF (420 mL, 5427.13 mmol, 38.39 equiv) was added oxalic acid (19.8 g, 219.91 mmol, 1.56 equiv) and Pd(OAc)₂ (3.2 g, 14.25 mmol, 0.10 equiv) and Xantphos (16.5 g, 28.51 mmol, 0.20 equiv) in portions at room temperature. The mixture was added DIEA (42 mL, 241.12 mmol, 1.71 equiv) dropwise at room temperature. The resulting mixture was stirred for 4 h at 100 degrees C. under nitrogen atmosphere. The mixture was allowed to cool down to room temperature. LCMS indicated the reaction was completed.

The resulting mixture was quenched with water (1 L) and extracted with EtOAc (3×800 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (1:1) to afford 3-(methoxycarbonyl)-4-methyl-5-(trifluoromethyl)benzoic acid (29 g, 78%) as an off-white solid. LCMS:(ES·m/z):261[M−1].

### Step 3: Synthesis of Compound 4

To a stirred mixture of 3-(methoxycarbonyl)-4-methyl-5-(trifluoromethyl) benzoic acid (39 g, 148.75 mmol, 1 equiv) in acetonitrile was added NBS (38.92 g, 218.66 mmol, 1.47 equiv.) and BPO (11.44 g, 44.62 mmol, 0.3 equiv.) in portions at 80° C. under nitrogen atmosphere for overnight. LCMS indicated reaction was completed. The mixture was allowed to cool down to room temperature. The resulting mixture was concentrated under reduced pressure. The resulting mixture was quenched with H₂O (300 mL) and extracted with EtOAc (3×300 mL). The combined organic layers were washed with brine (500 mL), dried over anhydrous Na₂SO₄. After filtration, the filtrate was concentrated under reduced pressure. The residue was purified by silica gel column chromatography, eluted with PE/EA (10:1) to afford 4-(bromomethyl)-3-(methoxycarbonyl)-5-(trifluoromethyl)benzoic acid (56.5 g, 95%) as a light yellow solid. LCMS (ESI, MIS): 339,341 [M−H]⁻

### Step 4: Synthesis of Compound 5

A solution of 4-(bromomethyl)-3-(methoxycarbonyl)-5-(trifluoromethyl)benzoic acid (20 g, 58.63 mmol, 1 equiv) in THE followed was added BH₃-THF (150 mL, 1N in THF, 150 mmol, 2.56 equiv) dropwise at 0° C. The resulting mixture was stirred for additional 2 h at room temperature. LCMS indicated the reaction was completed. The reaction was quenched by the addition of MeOH (5 mL) at room temperature. The resulting mixture was concentrated under reduced pressure. The residue was purified by silica gel column chromatography, eluted with PE/EA (4:1) to afford methyl 2-(bromomethyl)-5-(hydroxymethyl)-3-(trifluoromethyl) benzoate (15 g, 78%) as a yellow oil. LCMS: (ES, m/s): 245[M−1−Br]⁻.

### Step 5: Synthesis of Compound 6

To a stirred solution of methyl 2-(bromomethyl)-5-(hydroxymethyl)-3-(trifluoromethyl)benzoate (15 g, 45.85 mmol, 1 equiv.) were added NH₃ (7N in MeOH, 100 mL) in portions at room temperature under air atmosphere. The resulting mixture was stirred for additional 2 h at room temperature. LCMS indicated the reaction was completed. The resulting mixture was concentrated under reduced pressure to afford 6-(hydroxymethyl)-4-(trifluoromethyl)-2,3-dihydroisoindol-1-one (9.0 g, 84%) as a light brown solid. LCMS:(ES, MIS): 232 [M+H]⁺

### Step 6: Synthesis of Compound 7

The mixture of 6-(hydroxymethyl)-4-(trifluoromethyl)-2,3-dihydroisoindol-1-one (9 g, 38.93 mmol, 1 equiv) and IBX (32.70 g, 116.79 mmol, 3 equiv) in ethyl acetate was stirred for overnight at 60° C. LCMS indicated the reaction was completed. The mixture was allowed to cool down to room temperature. The resulting mixture was quenched with H₂O (300 mL) and extracted with EtOAc (3×200 mL). The combined organic layers were washed with brine (300 mL), dried over anhydrous Na₂SO₄. After filtration, the filtrate was concentrated under reduced pressure. The residue was purified by silica gel column chromatography, eluted with PE/EA (1:2) to afford 3-oxo-7-(trifluoromethyl)-1,2-dihydroisoindole-5-carbaldehyde (4.72 g, 41%) as a white solid. LCMS:(ES, M/S): 230 [M+H]⁺

### Step 7: Synthesis of Compound 8

To a stirred solution of 2,6-dichloro-4-{3-[(4-methyl-1,2,4-triazol-3-yl)methyl]oxetan-3-yl}pyridine (1 g, 3.34 mmol, 1 equiv) and R aminopropionitrile (4.17 g, 59.49 mmol, 17.80 equiv) in DMA were added K₂CO₃ (0.92 g, 6.70 mmol, 2.00 equiv) in portions at room temperature under air atmosphere. The resulting mixture was stirred for overnight at 120° C. LCMS indicated the reaction was completed. The mixture was allowed to cool down to room temperature and quenched with Water. The resulting mixture was extracted with EtOAc (3×30 mL). The combined organic layers were washed with brine (100 mL), dried over anhydrous Na₂SO₄. After filtration, the filtrate was concentrated under reduced pressure. The residue was purified by silica gel column chromatography, eluted with CH₂Cl₂/MeOH (8:1) to afford 3-[(6-chloro-4-{3-[(4-methyl-1,2,4-triazol-3-yl)methyl]oxetan-3-yl}pyridin-2-yl)amino]propanenitrile (610 mg, 49%) as an orange solid. LCMS (ES, m/s): 333,335 [M+H]⁺

### Step 8: Synthesis of Compound 157

To a stirred solution of 3-[(6-chloro-4-{3-[(4-methyl-1,2,4-triazol-3-yl)methyl]oxetan-3-yl}pyridin-2-yl)amino]propanenitrile (1.5 g, 4.50 mmol, 1 equiv) and 3-oxo-7-(trifluoromethyl)-1,2-dihydroisoindole-5-carbaldehyde (1.14 g, 4.95 mmol, 1.1 equiv) in dioxane (15 mL) was added Cs₂CO₃ (2.94 g, 9.01 mmol, 2 equiv), XantPhos (0.52 g, 0.90 mmol, 0.2 equiv) and Pd(OAc)₂ (0.10 g, 0.45 mmol, 0.1 equiv) at room temperature under nitrogen atmosphere. The resulting mixture was stirred for 3 h at 120° C. under nitrogen atmosphere. LCMS indicated the reaction was completed. The mixture was allowed to cool down to room temperature. The reaction mixture was purified by reverse flash chromatography with the following conditions: column, C18 silica gel; mobile phase, MeCN in Water (0.05% TFA), 5% to 60% gradient in 30 min; detector, UV 254 nm. The collected fraction was lyophilized to afford 3-({6-[6-formyl-1-oxo-4-(trifluoromethyl)-3H-isoindol-2-yl]-4-{3-[(4-methyl-1,2,4-triazol-3-yl)methyl]oxetan-3-yl}pyridin-2-yl}amino)propanenitrile (687.5 mg, 29%) as a yellow solid. LCMS (ES, m/s): 526[M+H−TFA]⁺H-NMR (300 MHz, DMSO-d₆): 10.21 (s, 1H), 8.72 (s, 1H), 8.56 (s, 1H), 7.48 (s, 1H), 7.15-1.11 (m, 1H), 6.13 (s, 1H), 5.37 (s, 2H), 4.91-4.75 (m, 4H), 3.62 (s, 2H), 3.51 (br s, 2H), 3.36 (s, 3H), 2.84 (t, J=6.6 Hz, 2H).

### Synthesis of Compound 158

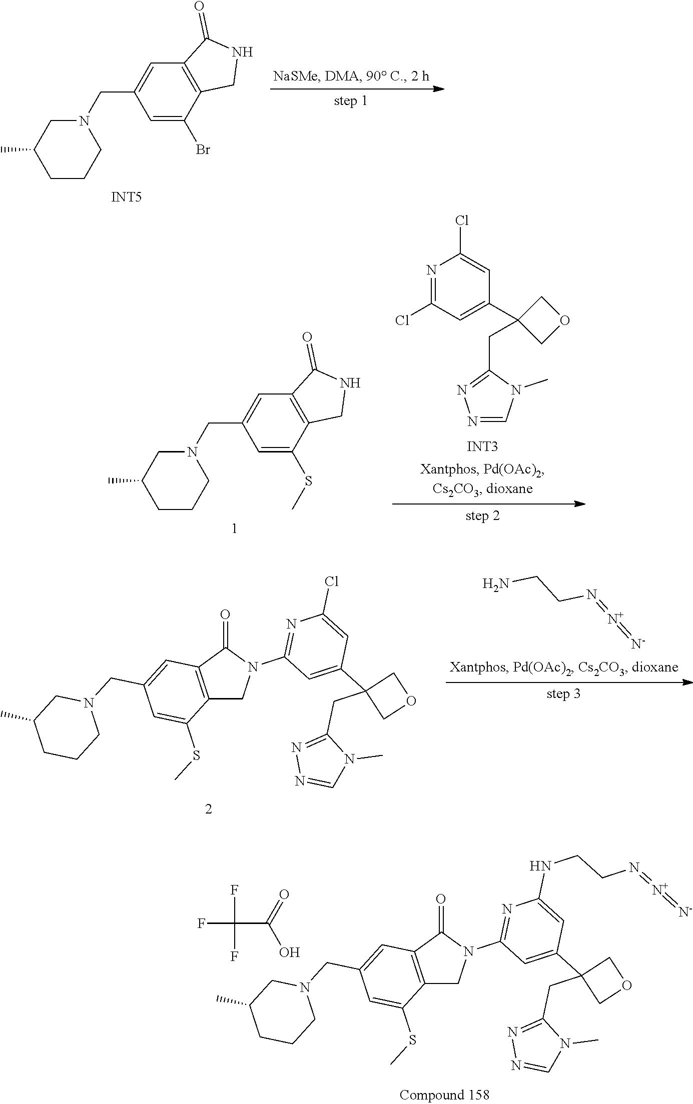

### Step 1: Synthesis of Compound 1

To a stirred solution of 4-bromo-6-{[(3S)-3-methylpiperidin-1-yl]methyl}-2,3-dihydroisoindol-1-one (500 mg, 1.54 mmol, 1 equiv) in DMF (5 mL) was added Sodiummethanethiolate (162 mg, 2.32 mmol, 1.5 equiv) in portions at room temperature under nitrogen atmosphere. The resulting mixture was stirred for 1 h at 90° C. under nitrogen atmosphere. LCMS indicated the reaction was completed. The mixture was allowed to cool down to room temperature. The residue was purified by reverse flash chromatography with the following conditions: column, C18 silica gel; mobile phase, MeCN in Water (0.05% TFA), 5% to 60% gradient in 30 min; detector, UV 254 nm. The collected fraction was concentrated to afford 6-{[(3S)-3-methylpiperidin-1-yl]methyl}-4-(methylsulfanyl)-2,3-dihydroisoindol-1-one (410 mg, 91%) as a white solid. LCMS (ESI, m/z):291[M+H]⁺

### Step 2: Synthesis of Compound 2

To a stirred solution of 6-{[(3S)-3-methylpiperidin-1-yl]methyl}-4-(methylsulfanyl)-2,3-dihydroisoindol-1-one (100 mg, 0.34 mmol, 1 equiv) and 2,6-dichloro-4-{3-[(4-methyl-1,2,4-triazol-3-yl)methyl]oxetan-3-yl}pyridine (113 mg, 0.37 mmol, 1.1 equiv) in dioxane (2 mL) was added Xantphos (39 mg, 0.069 mmol, 0.2 equiv), Cs₂CO₃ (224 mg, 0.68 mmol, 2 equiv) and Pd(OAc)₂ (8 mg, 0.034 mmol, 0.1 equiv) at room temperature under nitrogen atmosphere. The resulting mixture was stirred for 3 h at 90° C. under nitrogen atmosphere. LCMS indicated the reaction was completed. The mixture was allowed to cool down to room temperature. The reaction mixture was purified by reverse flash chromatography with the following conditions: column, C18 silica gel; mobile phase, MeCN in Water (0.05% TFA), 5% to 60% gradient in 30 min; detector, UV 254 nm. This resulted in 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(methylsulfanyl)-3H-isoindol-1-one (110 mg, 57%) as a white solid. LCMS (ESI, m/z):553,555[M+H]⁺

### Step 3: Synthesis of Compound 158

To a stirred solution of 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(methylsulfanyl)-3H-isoindol-1-one (100 mg, 0.18 mmol, 1 equiv) and 2-azidoethanamine (18 mg, 0.21 mmol, 1.2 equiv) in dioxane (200 uL) was added Cs₂CO₃ (117 mg, 0.36 mmol, 2 equiv) Xantphos (20 mg, 0.036 mmol, 0.2 equiv) and Pd(OAc)₂ (4 mg, 0.018 mmol, 0.1 equiv) at room temperature under nitrogen atmosphere. The resulting mixture was stirred for 2 h at 100° C. under nitrogen atmosphere. LCMS indicated the reaction was completed. The mixture was allowed to cool down to room temperature. The reaction mixture was purified by reverse flash chromatography with the following conditions: column, C18 silica gel; mobile phase, MeCN in Water (0.05% TFA), 5% to 60% gradient in 10 min; detector, UV 254 nm. to afford crude product as a colorless solid. The crude product (20 mg) was purified by Prep-HPLC with the following conditions (Column: XBridge Prep Phenyl OBD Column, 19*150 mm, 5 m; Mobile Phase A: Water (0.1% TFA), Mobile Phase B: MeOH--HPLC; Flow rate: 25 mL/min; Gradient: 45% B to 75% B in 10 min. 75% B; Wave Length: 254 nm; RT1 (min): 7.32; The collected fraction was lyophilized to afford 2-{6-[(2-azidoethyl)amino]-4-{3-[(4-methyl-1,2,4-triazol-3-yl)methyl]oxetan-3-yl}pyridin-2-yl}-6-{[(3S)-3-methylpiperidin-1-yl]methyl}-4-(methylsulfanyl)-3H-isoindol-1-one; trifluoroacetic acid (5.1 mg, 3.87%) as a white solid. LCMS (ESI, m/z):603[M+H−TFA]⁺; ¹H NMR (300 MHz, DMSO-d₆) δ 9.88-9.95 (m, 1H), 8.52-8.50 (m, 1H), 7.74-7.70 (m, 2H), 7.43-6.96 (m, 2H), 6.05 (s, 1H), 4.92-4.89 (m, 4H), 4.79-4.72 (m, 2H), 4.67-4.59 (m, 2H), 3.56-3.49 (, 6H), 3.39-3.35 (m, 1H), 3.26-3.00 (m, 1H), 2.89-78 (m, 4H), 2.63-2.51 (m, 4H), 1.85-1.59 (m, 4H), 1.04-1.00 (m, 1H), 0.89 (d, J=6.6 Hz, 3H).

### Synthesis of Compound 159

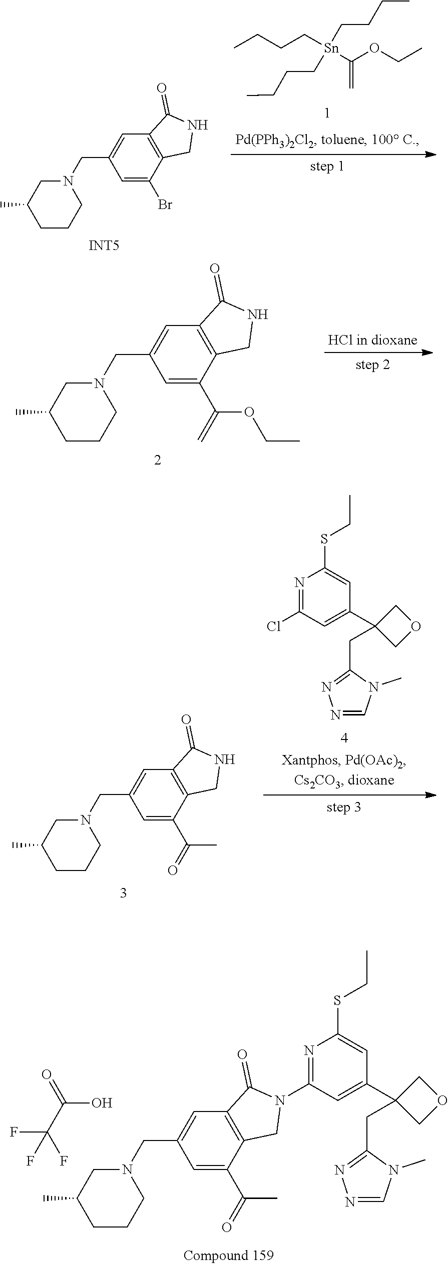

### Step 1: Synthesis of Compound 2

A mixture of 4-bromo-6-{[(3S)-3-methylpiperidin-1-yl]methyl}-2,3-dihydroisoindol-1-one (300 mg, 0.92 mmol, 1 equiv), Pd(PPh₃)₂C₁₂ (65 mg, 0.093 mmol, 0.1 equiv) and tributyl (1-ethoxyethenyl)stannane (402 mg, 1.11 mmol, 1.2 equiv) in Toluene (5 mL) was stirred for 4 h at 90° C. under nitrogen atmosphere. LCMS indicated the reaction was completed. The reaction was allowed to cool down to room temperature. The solvent was removed under reduced pressure. The residue was purified by Prep-TLC (DCM/MeOH=10:1) to afford 4-acetyl-6-{[(3S)-3-methylpiperidin-1-yl]methyl}-2,3-dihydroisoindol-1-one) as a yellow solid. LCMS (ESI, m/z): 315 [M+H]⁺

### Step 2: Synthesis of Compound 3

The solution of 4-(1-ethoxyethenyl)-6-{[(3S)-3-methylpiperidin-1-yl]methyl}-2,3-dihydroisoindol-1-one (200 mg, 0.63 mmol, 1 equiv) in HCl (2N, 3 mL) and THF (1.5 mL) was stirred for 2 h at 60° C. LCMS indicated the reaction was completed. The reaction was allowed to cool down to room temperature. The resulting mixture was concentrated under reduced pressure. The residue was purified by silica gel column chromatography, eluted with CH₂Cl₂/MeOH (10:1) to afford 4-acetyl-6-{[(3S)-3-methylpiperidin-1-yl]methyl}-2,3-dihydroisoindol-1-one (110 mg, 54%) as a white solid. LCMS: (MS, E/S): 287 [M+H]⁺

### Step 3: Synthesis of Compound 159

To a stirred solution of 4-acetyl-6-{[(3S)-3-methylpiperidin-1-yl]methyl}-2,3-dihydroisoindol-1-one (110 mg, 0.38 mmol, 1 equiv.) and 2-chloro-6-(ethylsulfanyl)-4-{3-[(4-methyl-1,2,4-triazol-3-yl)methyl]oxetan-3-yl}pyridine (137 mg, 0.42 mmol, 1.1 equiv) in 1,4-dioxane were added Pd(OAc)₂ (8 mg, 0.038 mmol, 0.10 equiv), Cs₂CO₃ (250 mg, 0.76 mmol, 2 equiv) and XantPhos (44 mg, 0.077 mmol, 0.2 equiv) in portions at room temperature under nitrogen atmosphere. The mixture was stirred for 2 h at 90° C. LCMS indicated the reaction was completed. The reaction was allowed to cool down to room temperature. The residue was purified by reverse flash chromatography with the following conditions: column, C18 silica gel; mobile phase, water (0.05% TFA), ACN 5% to 50% gradient in 30 min; detector, UV 254 nm. The collected fraction was lyophilized to afford 4-acetyl-2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-3H-isoindol-1-one; trifluoroacetic acid (20 mg, 7%) as a light yellow solid. LCMS:(ES, MIS): 575 [M+H−TFA]⁺; ¹H-NMR (300 MHz, DMSO-d₆): 10.67 (br s, 1H), 8.78-8.66 (m, 2H), 8.22 (s, 1H), 8.06 (s, 1H), 6.99 (s, 1H), 5.33 (br s, 2H), 4.92-4.84 (m, 4H), 4.54 (s, 2H), 3.68 (s, 2H), 3.55-3.37 (m, 3H), 3.30 (d, J=9.9 Hz, 1H), 3.22-3.15 (m, 2H), 2.92-2.90 (m, 1H), 2.80-2.53 (m, 5H), 2.02-1.88 (m, 1H), 1.81-1.73 (m, 3H), 1.41 (t, J=6.9 Hz, 3H), 1.15-1.07 (m, 1H), 0.89 (d, J=6 Hz, 3H).

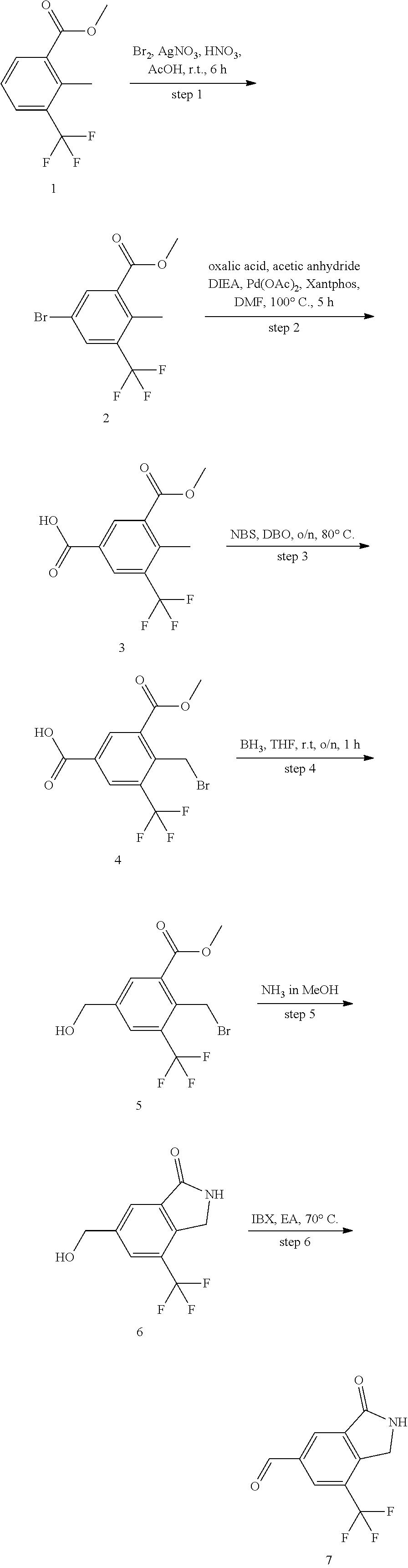

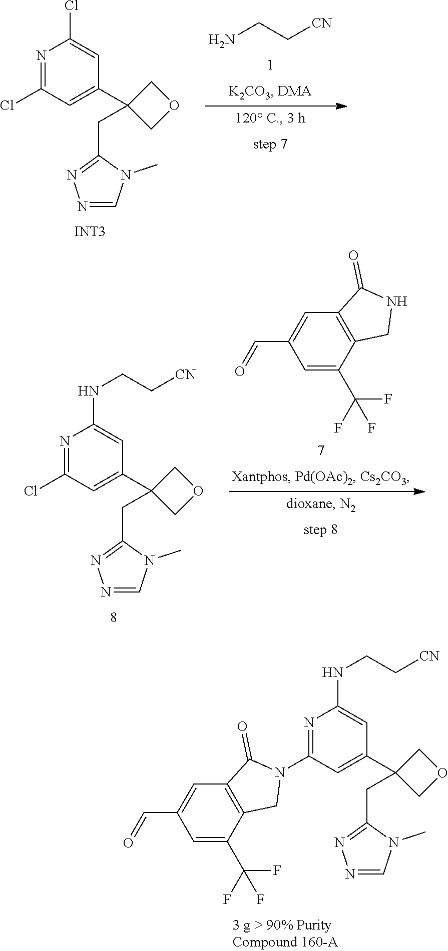

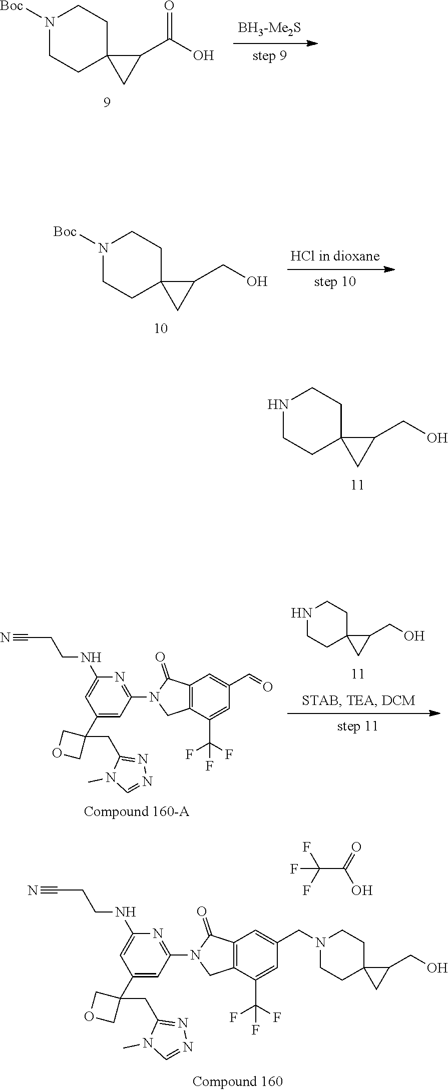

### Step 1: Synthesis of Compound 2

To a stirred mixture of methyl 2-methyl-3-(trifluoromethyl)benzoate (44 g, 201.67 mmol, 1 equiv) in AcOH (286 mL) was added HNO₃ (127 g, 2015.46 mmol, 9.99 equiv) dropwise over 20 min at 10° C. under nitrogen atmosphere. To the above mixture was added Br₂ (35.5 g, 222.14 mmol, 1.10 equiv) dropwise over 10 min at 10° C. The resulting mixture was stirred for additional 10 min at 10° C. To the above mixture was added AgNO₃ (44.4 g, 261.37 mmol, 1.30 equiv) in water (105 mL) (2.5 mol/L) dropwise over 25 min at 10° C. The resulting mixture was stirred for additional overnight at room temperature. TLC indicated the reaction was completed. The mixture was basified to pH 8 with saturated Na₂CO₃ (aq.). The resulting mixture was extracted with EtOAc (3×800 mL). The combined organic layers were washed with water (3×100 mL), dried over anhydrous Na₂SO₄. After filtration, the filtrate was concentrated under reduced pressure. The residue was purified by silica gel column chromatography, eluted with PE/EA (10:1) to afford methyl 5-bromo-2-methyl-3-(trifluoromethyl)benzoate (42 g, 70%) as a yellow oil. ¹H NMR (300 MHz, DMSO) δ 8.32 (s, 1H), 8.08 (s, 1H), 3.87 (s, 3H), 3.46-3.44 (m, 3H).

### Step 2: Synthesis of Compound 3

To a stirred mixture of methyl 5-bromo-2-methyl-3-(trifluoromethyl)benzoate (42 g, 141.38 mmol, 1.00 equiv) and acetic anhydride (21.7 g, 212.56 mmol, 1.50 equiv) in DMF (420 mL, 5427.13 mmol, 38.39 equiv) was added oxalic acid (19.8 g, 219.91 mmol, 1.56 equiv) and Pd(OAc)₂ (3.2 g, 14.25 mmol, 0.10 equiv) and Xantphos (16.5 g, 28.51 mmol, 0.20 equiv) in portions at room temperature. The mixture was added DIEA (42 mL, 241.12 mmol, 1.71 equiv) dropwise at room temperature. The resulting mixture was stirred for 4 h at 100 degrees C. under nitrogen atmosphere. The mixture was allowed to cool down to room temperature. LCMS indicated the reaction was completed.

The resulting mixture was quenched with water (1 L) and extracted with EtOAc (3×800 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (1:1) to afford 3-(methoxycarbonyl)-4-methyl-5-(trifluoromethyl)benzoic acid (29 g, 78%) as an off-white solid. LCMS:(ES·m/z):261[M−1]⁻.

### Step 3: Synthesis of Compound 4

To a stirred mixture of 3-(methoxycarbonyl)-4-methyl-5-(trifluoromethyl) benzoic acid (39 g, 148.75 mmol, 1 equiv) in acetonitrile was added NBS (38.92 g, 218.66 mmol, 1.47 equiv.) and BPO (11.44 g, 44.62 mmol, 0.3 equiv.) in portions at 80° C. under nitrogen atmosphere for overnight. LCMS indicated reaction was completed. The mixture was allowed to cool down to room temperature. The resulting mixture was concentrated under reduced pressure. The resulting mixture was quenched with H₂O (300 mL) and extracted with EtOAc (3×300 mL). The combined organic layers were washed with brine (500 mL), dried over anhydrous Na₂SO₄. After filtration, the filtrate was concentrated under reduced pressure. The residue was purified by silica gel column chromatography, eluted with PE/EA (10:1) to afford 4-(bromomethyl)-3-(methoxycarbonyl)-5-(trifluoromethyl)benzoic acid (56.5 g, 95%) as a light yellow solid. LCMS (ESI, MIS): 339,341 [M−H]⁻

### Step 4: Synthesis of Compound 5

A solution of 4-(bromomethyl)-3-(methoxycarbonyl)-5-(trifluoromethyl)benzoic acid (20 g, 58.63 mmol, 1 equiv) in THE followed was added BH₃-THF (150 mL, 1N in THF, 150 mmol, 2.56 equiv) dropwise at 0° C. The resulting mixture was stirred for additional 2 h at room temperature. LCMS indicated the reaction was completed. The reaction was quenched by the addition of MeOH (5 mL) at room temperature. The resulting mixture was concentrated under reduced pressure. The residue was purified by silica gel column chromatography, eluted with PE/EA (4:1) to afford methyl 2-(bromomethyl)-5-(hydroxymethyl)-3-(trifluoromethyl) benzoate (15 g, 78%) as a yellow oil. LCMS: (ES, m/s): 245[M−1−Br]⁻.

### Step 5: Synthesis of Compound 6

To a stirred solution of methyl 2-(bromomethyl)-5-(hydroxymethyl)-3-(trifluoromethyl)benzoate (15 g, 45.85 mmol, 1 equiv.) were added NH₃ (7N in MeOH, 100 mL) in portions at room temperature under air atmosphere. The resulting mixture was stirred for additional 2 h at room temperature. LCMS indicated the reaction was completed. The resulting mixture was concentrated under reduced pressure to afford 6-(hydroxymethyl)-4-(trifluoromethyl)-2,3-dihydroisoindol-1-one (9.0 g, 84%) as a light brown solid. LCMS:(ES, M/S): 232 [M+H]⁺

### Step 6: Synthesis of Compound 7

The mixture of 6-(hydroxymethyl)-4-(trifluoromethyl)-2,3-dihydroisoindol-1-one (9 g, 38.93 mmol, 1 equiv) and IBX (32.70 g, 116.79 mmol, 3 equiv) in ethyl acetate was stirred for overnight at 60° C. LCMS indicated the reaction was completed. The mixture was allowed to cool down to room temperature. The resulting mixture was quenched with H₂O (300 mL) and extracted with EtOAc (3×200 mL). The combined organic layers were washed with brine (300 mL), dried over anhydrous Na₂SO₄. After filtration, the filtrate was concentrated under reduced pressure. The residue was purified by silica gel column chromatography, eluted with PE/EA (1:2) to afford 3-oxo-7-(trifluoromethyl)-1,2-dihydroisoindole-5-carbaldehyde (4.72 g, 41%) as a white solid. LCMS:(ES, M/S): 230 [M+H]⁺

### Step 7: Synthesis of Compound 8

To a stirred solution of 2,6-dichloro-4-{3-[(4-methyl-1,2,4-triazol-3-yl)methyl]oxetan-3-yl}pyridine (1 g, 3.34 mmol, 1 equiv) and R aminopropionitrile (4.17 g, 59.49 mmol, 17.80 equiv) in DMA were added K₂CO₃ (0.92 g, 6.70 mmol, 2.00 equiv) in portions at room temperature under air atmosphere. The resulting mixture was stirred for overnight at 120° C. LCMS indicated the reaction was completed. The mixture was allowed to cool down to room temperature and quenched with Water. The resulting mixture was extracted with EtOAc (3×30 mL). The combined organic layers were washed with brine (100 mL), dried over anhydrous Na₂SO₄. After filtration, the filtrate was concentrated under reduced pressure. The residue was purified by silica gel column chromatography, eluted with CH₂Cl₂/MeOH (8:1) to afford 3-[(6-chloro-4-{3-[(4-methyl-1,2,4-triazol-3-yl)methyl]oxetan-3-yl}pyridin-2-yl)amino]propanenitrile (610 mg, 49%) as an orange solid. LCMS (ES, m/s): 333,335 [M+H]⁺

### Step 8: Synthesis of Compound 160-A

To a stirred mixture of 3-oxo-7-(trifluoromethyl)-1,2-dihydroisoindole-5-carbaldehyde (207 mg, 0.90 mmol, 1.0 equiv) and 3-[(6-chloro-4-{3-[(4-methyl-1,2,4-triazol-3-yl)methyl]oxetan-3-yl}pyridin-2-yl)amino]propanenitrile (300 mg, 0.90 mmol, 1.0 equiv) in 1,4-dioxane was added Xantphos (106 mg, 0.18 mmol, 0.20 equiv), Pd(OAc)₂ (2 mg, 0.09 mmol, 0.1 equiv) and Cs₂CO₃ (53 mg, 0.18 mmol, 2 equiv) in portions at room temperature under air atmosphere. The mixture was stirred for 3 h at 120° C. under nitrogen atmosphere. LCMS indicated the reaction was completed. The mixture was allowed to cool down to room temperature and concentrated to dryness under vacuum. The residue was purified by silica gel column chromatography, eluted with CH₂Cl₂/MeOH (10:1) to afford 3-({6-[6-formyl-1-oxo-4-(trifluoromethyl)-3H-isoindol-2-yl]-4-{3-[(4-methyl-1,2,4-triazol-3-yl)methyl]oxetan-3-yl}pyridin-2-yl}amino)propanenitrile (170 mg, 31%) as an orange solid. LCMS (ms, E/S): 526 [M+H]⁺

### Step 9: Synthesis of Compound 10

To a stirred mixture of 6-(tert-butoxycarbonyl)-6-azaspiro[2.5]octane-1-carboxylic acid (1.0 g, 3.91 mmol, 1 equiv) and BH₃-MesS (1.20 mL, ION in MesS, 12.0 mmol, 3.06 equiv) in THE at room temperature under air atmosphere. The mixture was stirred for 3 h at 70° C. under air atmosphere. TLC indicated the reaction was completed. The mixture was allowed to cool down to room temperature. The reaction was quenched with Water at room temperature. The resulting mixture was concentrated under reduced pressure. The residue was purified by silica gel column chromatography, eluted with PE/EA (1:1) to afford tert-butyl 1-(hydroxymethyl)-6-azaspiro[2.5]octane-6-carboxylate (810 mg, 77%) as a colorless oil. LCMS (MS, E/S): 242 [M+H]⁺

### Step 10: Synthesis of Compound 11

The mixture of tert-butyl 1-(hydroxymethyl)-6-azaspiro [2.5]octane-6-carboxylate (400 mg, 1.65 mmol, 1 equiv) in HCl (4N in dioxane, 4 mL) was stirred at room temperature for 2 h. LCMS indicated the reaction was completed. The resulting mixture was concentrated under reduced pressure. The crude product was used to next step without purification. LCMS (MS, E/S): 142 [M+H]⁺

### Step 11: Synthesis of Compound 160

To a stirred solution of 3-({6-[6-formyl-1-oxo-4-(trifluoromethyl)-3H-isoindol-2-yl]-4-{3-[(4-methyl-1,2,4-triazol-3-yl)methyl]oxetan-3-yl}pyridin-2-yl}amino)propanenitrile (100 mg, 0.19 mmol, 1 equiv) and 6-azaspiro[2.5]octan-1-ylmethanol (80 mg, 0.56 mmol, 2.98 equiv) in DCM were added TEA (38 mg, 0.38 mmol, 2 equiv) and STAB (160 mg, 0.75 mmol, 3.97 equiv) in portions at room temperature under air atmosphere. The mixture was stirred for overnight at room temperature. LCMS indicated the reaction was completed. The reaction was quenched with MeOH at ° C. The resulting mixture was concentrated under vacuum. The residue was purified by reverse flash chromatography with the following conditions: column, C18 silica gel; mobile phase, MeCN in Water (0.05% TFA), 5% to 60% gradient in 30 min; detector, UV 254 nm. The eluted was concentrated under vacuum to dryness. The crude product (25 mg) was purified by Prep-HPLC with the following conditions (Column: XBridge Shield RP18 OBD Column, 19*150 mm, 5 m; Mobile Phase A: Water (0.1% TFA), Mobile Phase B: ACN; Flow rate: 25 mL/min; Gradient: 8% B to 39% B in 7 min. 39% B; Wave Length: 254 nm; RT1 (min): 5.58; The eluted was lyophilized to afford 3-{[6-(6-{[1-(hydroxymethyl)-6-azaspiro[2.5]octan-6-yl]methyl}-1-oxo-4-(trifluoromethyl)-3H-isoindol-2-yl)-4-{3-[(4-methyl-1,2,4-triazol-3-yl)methyl]oxetan-3-yl}pyridin-2-yl]amino}propanenitrile (8.8 mg, 7%) as a white solid. H-NMR (300 MHz, DMSO-d₆): 9.74 (br s, 1H), 8.44 (s, 1H), 8.28-8.21 (m, 2H), 7.49 (s, 1H), 7.16-7.11 (m, 1), 6.11 (s, 1H), 5.38 (s, 2H), 4.94 (d, J=6 Hz, 2H), 4.77 (d, J=6 Hz, 2H), 4.62-4.57 (m, 2H), 3.57-3.50 (m, 3H), 3.40-3.35 (m, 2H), 3.28 (s, 3H), 3.23-3.20 (m, 2H), 3.16-2.95 (m, 2H), 2.83 (t, J=6.6 Hz, 2H), 2.04-1.95 (m, 2H), 1.58-1.46 (m, 1H), 1.17-1.05 (m, 1H), 1.01-0.94 (m, 1H), 0.57-0.52 (m, 1H), 0.28-0.26 (m, 1H).

### Synthesis of Compound 161

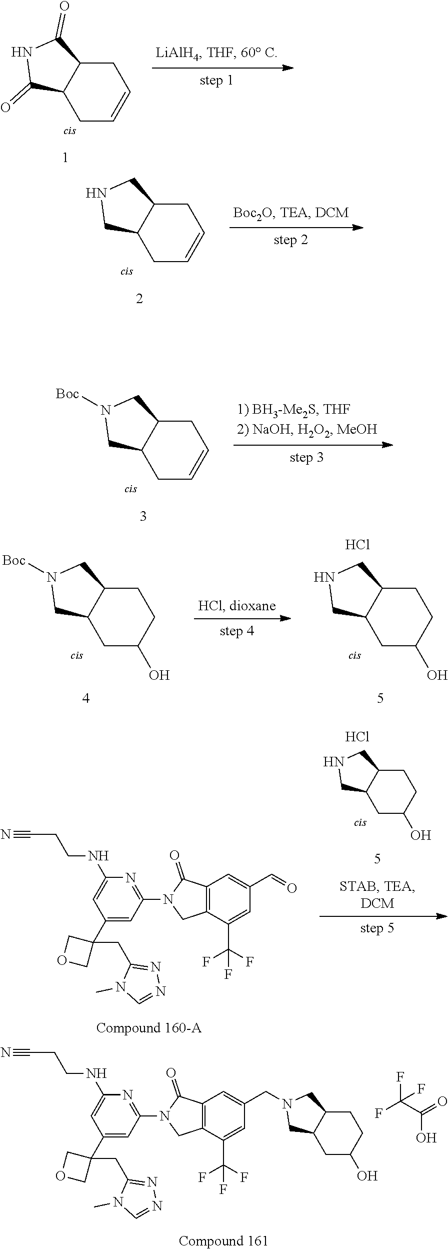

### Step 1: Synthesis of Compound 2

To a stirred solution of LiAlH₄ (2.40 g, 63.14 mmol, 2.3 equiv) in THE (60 mL) was added (3aR,7aS)-3a,4,7,7a-tetrahydro-2H-isoindole-1,3-dione (4.15 g, 27.45 mmol, 1 equiv) in portions at 0° C. under nitrogen atmosphere. The resulting mixture was stirred for overnight at 60° C. under nitrogen atmosphere. LCMS indicated the reaction was completed. The reaction was quenched by the addition of water (3 mL) and NaOH (1N, 3 mL) at 0° C. The mixture was stirred at r.t. for 1 h, and then filtered through a Celite pad, which washed with DCM (3×5 mL). The filtrate was concentrated under reduced pressure. This resulted in (3aR,7aS)-2,3,3a,4,7,7a-hexahydro-1H-isoindole (3 g, 88%) as a yellow oil. LCMS (ESI, m/z):124[M+H]⁺

### Step 2: Synthesis of Compound 3

To a stirred solution of (3aR,7aS)-2,3,3a,4,7,7a-hexahydro-1H-isoindole (3 g, 24.35 mmol, 1 equiv) and TEA (4.93 g, 48.70 mmol, 2 equiv) in DCM (30 mL) was added Boc₂O (6.38 g, 29.22 mmol, 1.2 equiv) in portions at 0° C. under nitrogen atmosphere. The resulting mixture was stirred for overnight at room temperature under nitrogen atmosphere. LCMS indicated the reaction was completed. The reaction was quenched by the addition of water (30 mL) and extracted with DCM (30 mL*3). The organic layer was washed with brine (30 mL) and dried with Na₂SO₄. The filtrate was concentrated under vacuum. The residue was purified by silica gel column chromatography, eluted with PE/EA (5:1) to afford tert-butyl (3aR,7aS)-1,3,3a,4,7,7a-hexahydroisoindole-2-carboxylate (4.5 g, 82%) as an off-white solid. LCMS (ESI, m/z):168[M+H−56]⁺

### Step 3: Synthesis of Compound 4

To a stirred solution of tert-butyl (3aR,7aS)-1,3,3a,4,7,7a-hexahydroisoindole-2-carboxylate (2 g, 8.95 mmol, 1 equiv) in dry THF (35 mL) was added BH₃-Me₂S (4.48 mL, 8.95 mmol, 1 equiv) dropwise at 0° C. under nitrogen atmosphere and stirred overnight. The mixture was cooled to 0° C., and then methanol (2 mL) was added dropwise, followed by a mixture of aqueous NaOH (1 mL, 3 N) and H₂O₂ (1 mL, 30%). The mixture was stirred at 60° C. for 1.5 h. LCMS indicated the reaction was completed. The resulting mixture was diluted with water (50 mL). The resulting mixture was extracted with EtOAc (3×50 mL). The combined organic layers were washed with brine (50 mL), dried over anhydrous Na₂SO₄. After filtration, the filtrate was concentrated under reduced pressure. This resulted in tert-butyl (3aR,7aS)-5-hydroxy-octahydroisoindole-2-carboxylate (700 mg, 32%) as a yellow oil. LCMS (ESI, m/z):186[M+H−56]⁺

### Step 4: Synthesis of Compound 5

To a stirred solution of tert-butyl (3aR,7aS)-5-hydroxy-octahydroisoindole-2-carboxylate (700 mg, 2.90 mmol, 1 equiv) in DCM (3 mL) was added HCl (4N in 1,4-dioxane, 7 mL) dropwise at 0° C. under nitrogen atmosphere. The resulting mixture was stirred for 2 h at 0° C. under nitrogen atmosphere. LCMS indicated the reaction was completed. The resulting mixture was concentrated under vacuum. The crude product (600 mg) was purified by Prep-HPLC with the following conditions (Column: XBridge Prep Amide OBD Column, 19*150 mm, 5 m; Mobile Phase A: Water (0.05% HCl), Mobile Phase B: ACN; Flow rate: 25 mL/min; Gradient: 90% B to 70% B in 10 min. 70% B; Wave Length: 254 nm; RT1 (min): 7.2; The eluted was concentrated to afford (3aR,7aS)-octahydro-1H-isoindol-5-ol hydrochloride (300 mg, 58%) as an off-white solid. LCMS (ESI, m/z):142[M+H−HCl]⁺

### Step 5: Synthesis of Compound 161

To a stirred solution of 3-({6-[6-formyl-1-oxo-4-(trifluoromethyl)-3H-isoindol-2-yl]-4-{3-[(4-methyl-1,2,4-triazol-3-yl)methyl]oxetan-3-yl}pyridin-2-yl}amino)propanenitrile (100 mg, 0.19 mmol, 1 equiv) and (3aR,7aS)-octahydro-1H-isoindol-5-ol (53 mg, 0.38 mmol, 2 equiv) in DCM (2 mL) was added TEA (38 mg, 0.38 mmol, 2 equiv) dropwise at room temperature under nitrogen atmosphere. The resulting mixture was stirred for 30 min at room temperature under nitrogen atmosphere. To the above mixture was added STAB (161 mg, 0.76 mmol, 4 equiv) in portions over at room temperature. The resulting mixture was stirred for additional 2 h at room temperature. LCMS indicated the reaction was completed. The reaction was quenched with MeOH at ° C. The resulting mixture was concentrated under vacuum. The residue was purified by reverse flash chromatography with the following conditions: column, C18 silica gel; mobile phase, MeCN in Water (0.05% TFA), 5% to 60% gradient in 30 min; detector, UV 254 nm. The eluted was concentrated to afford 3-{[6-(6-{[(3aR,7aS)-5-hydroxy-octahydroisoindol-2-yl]methyl}-1-oxo-4-(trifluoromethyl)-3H-isoindol-2-yl)-4-{3-[(4-methyl-1,2,4-triazol-3-yl)methyl]oxetan-3-yl}pyridin-2-yl]amino}propanenitrile; trifluoroacetic acid (25 mg, 16%) as a light yellow solid. LCMS (ESI, m/z):651[M+H−TFA]⁺; ¹H NMR (300 MHz, DMSO-d₆) δ 10.27 (br s, 1H), 8.48 (s, 1H), 8.36-8.27 (m, 2H), 7.49 (s, 1H), 7.13 (br s, 1H), 6.12 (s, 1H), 5.26 (s, 2H), 4.91 (d, J=6.0 Hz, 2H), 4.80 (d, J=6.3 Hz, 2H), 4.65-4.59 (m, 2H), 3.72-3.51 (m, 5H), 3.34-3.32 (m, 4H), 3.28-3.08 (m, 3H), 2.82 (t, J=6.3 Hz, 2H), 2.42-5-2.42 (m, 1H), 2.25-2.20 (m, 1H), 1.86-1.45 (m, 5H), 1.27-1.23 (m, 2H).

### Synthesis of Compound 162

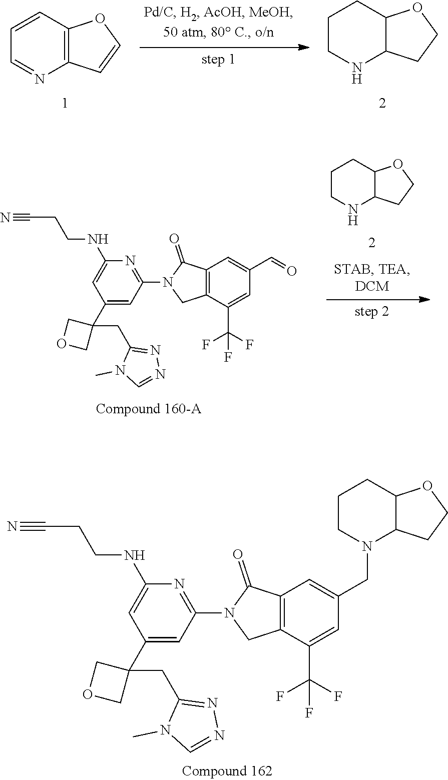 [SMILES: c1cnc2ccoc2c1 ; C1CNC2CCOC2C1 ; C1CNC2CCOC2C1 ; Cn1cnnc1CC1(c2cc(NCCC#N)nc(N3Cc4c(cc(C=O)cc4C(F)(F)F)C3=O)c2)COC1 ; Cn1cnnc1CC1(c2cc(NCCC#N)nc(N3Cc4c(cc(CN5CCCC6OCCC65)cc4C(F)(F)F)C3=O)c2)COC1]

### Step 1: Synthesis of Compound 2

To a solution of furo[3,2-b]pyridine (200 mg, 1.67 mmol, 1 equiv) in 10 mL MeOH and AcOH (2 mL) was added Pd/C (10%, 0.5 g) in a pressure tank. The mixture was stirred under 50 psi of hydrogen pressure for overnight at 80° C. LCMS indicated the reaction was completed. The reaction was filtered through a Celite pad and concentrated under reduced pressure. This resulted in octahydrofuro[3,2-b]pyridine (100 mg, 46%) as a brown oil. LCMS (ESI, m/z):128[M+H]⁺

### Step 2: Synthesis of Compound 162

To a stirred solution of 3-({6-[6-formyl-1-oxo-4-(trifluoromethyl)-3H-isoindol-2-yl]-4-{3-[(4-methyl-1,2,4-triazol-3-yl)methyl]oxetan-3-yl}pyridin-2-yl}amino)propanenitrile (50 mg, 0.09 mmol, 1 equiv), TEA (19 mg, 0.19 mmol, 2 equiv) and octahydrofuro[3,2-b]pyridine (14 mg, 0.11 mmol, 1.2 equiv) in DCM (1 mL) was stirred for 30 min at room temperature under nitrogen atmosphere. To the above mixture was added STAB (80 mg, 0.38 mmol, 4 equiv) in portions at room temperature. The resulting mixture was stirred for additional overnight at room temperature. LCMS indicated the reaction was completed. The reaction was quenched with MeOH at 0° C. The resulting mixture was concentrated under vacuum. The residue was purified by reverse flash chromatography with the following conditions: column, C18 silica gel; mobile phase, ACN in water (0.05% TFA), 0% to 50% gradient in 30 min; detector, UV 254 nm and 220 nm. The crude product (30 mg) was purified by Prep-HPLC with the following conditions (Column: XBridge Shield RP18 OBD Column, 30*150 mm, 5 m; Mobile Phase A: Water (10 mmol/L NH₄HCO₃), Mobile Phase B: ACN; Flow rate: 60 mL/min; Gradient: 30% B to 60% B in 7 min, 60% B; Wave Length: 254 nm; RT1 (min): 5.32; The collected fraction was lyophilized to afford 3-{[6-(6-{hexahydro-2H-furo[3,2-b]pyridin-4-ylmethyl}-1-oxo-4-(trifluoromethyl)-3H-isoindol-2-yl)-4-{3-[(4-methyl-1,2,4-triazol-3-yl)methyl]oxetan-3-yl}pyridin-2-yl]amino}propanenitrile (4.5 mg, 7%) as an off-white solid. LCMS (ESI, m/z):637[M+H]⁺; ¹H NMR (300 MHz, DMSO-d₆) δ 8.24 (s, 1H), 7.95-7.90 (m, 2H), 7.49 (s, 1H), 7.07 (t, J=5.7 Hz, 1H), 6.06 (s, 1H), 5.19 (s, 2H), 4.91 (d, J=6.0 Hz, 2H), 4.79 (d, J=6.0 Hz, 2H), 4.10-3.93 (m, 2H), 3.82-3.74 (m, 2H), 3.53-3.44 (m, 5H), 3.25 (s, 3H), 2.83-2.73 (m, 3H), 2.56-2.52 (m, 1H), 2.13-2.11 (m, 1H), 2.08-1.87 (m, 2H), 1.81-1.78 (m, 1H), 1.58-1.52 (m, 2H), 1.48-1.32 (m, 1H).

### Synthesis of Compound 163

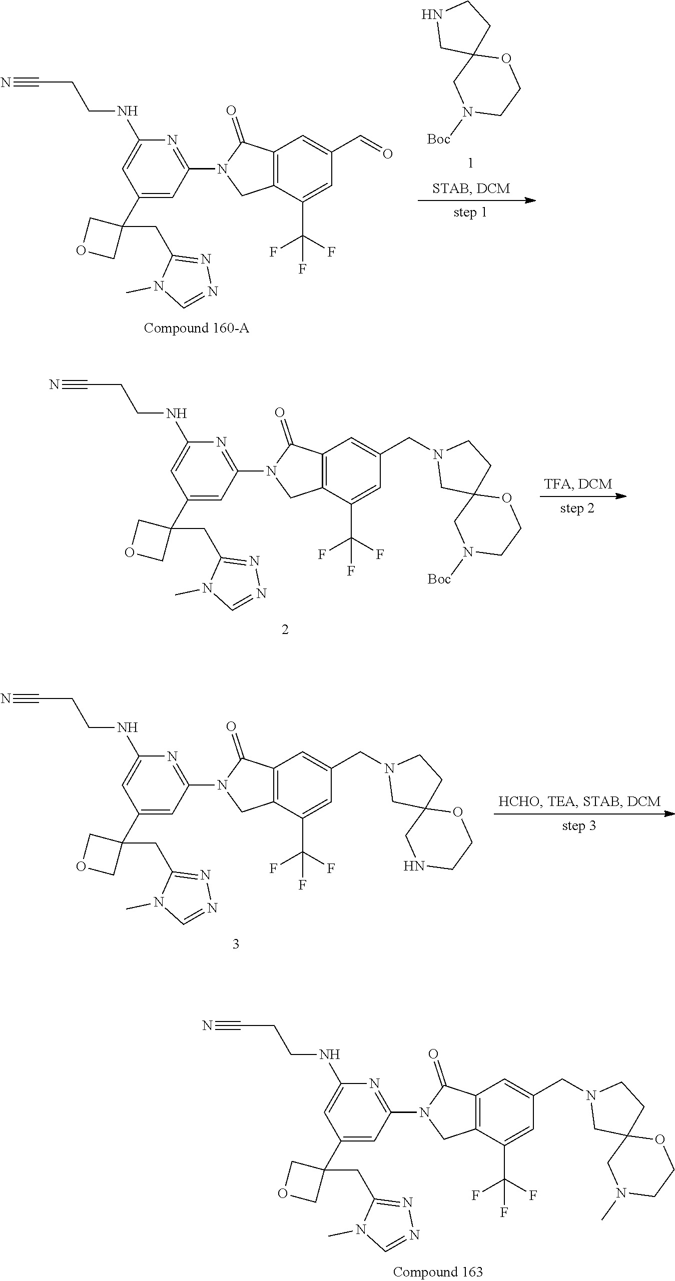 [SMILES: Cn1cnnc1CC1(c2cc(NCCC#N)nc(N3Cc4c(cc(C=O)cc4C(F)(F)F)C3=O)c2)COC1 ; CC(C)(C)OC(=O)N1CCOC2(CCNC2)C1 ; Cn1cnnc1CC1(c2cc(NCCC#N)nc(N3Cc4c(cc(CN5CCC6(C5)CN(C(=O)OC(C)(C)C)CCO6)cc4C(F)(F)F)C3=O)c2)COC1 ; Cn1cnnc1CC1(c2cc(NCCC#N)nc(N3Cc4c(cc(CN5CCC6(CNCCO6)C5)cc4C(F)(F)F)C3=O)c2)COC1 ; CN1CCOC2(CCN(Cc3cc4c(c(C(F)(F)F)c3)CN(c3cc(C5(Cc6nncn6C)COC5)cc(NCCC#N)n3)C4=O)C2)C1]

### Step 1: Synthesis of Compound 2

To a stirred mixture of 3-({6-[6-formyl-1-oxo-4-(trifluoromethyl)-3H-isoindol-2-yl]-4-{3-[(4-methyl-1,2,4-triazol-3-yl)methyl]oxetan-3-yl}pyridin-2-yl}amino)propanenitrile (100 mg, 0.19 mmol, 1 equiv) and tert-butyl 6-oxa-2,9-diazaspiro[4.5]decane-9-carboxylate (55 mg, 0.22 mmol, 1.2 equiv) in DCM (2 mL). The resulting mixture was stirred for 10 min under air atmosphere. To the above mixture was added STAB (161 mg, 0.76 mmol, 4 equiv) in portions at room temperature. The resulting mixture was stirred for additional 2 h at room temperature. LCMS indicated the reaction was completed. The resulting mixture was concentrated under reduced pressure. The residue was purified by reverse flash chromatography with the following conditions: column, C18 silica gel; mobile phase, TFA in Water (0.05% TFA), 0% to 50% gradient in 40 min; detector, UV 254 nm. to afford tert-butyl 2-[(2-{6-[(2-cyanoethyl)amino]-4-{3-[(4-methyl-1,2,4-triazol-3-yl)methyl]oxetan-3-yl}pyridin-2-yl}-3-oxo-7-(trifluoromethyl)-1H-isoindol-5-yl)methyl]-6-oxa-2,9-diazaspiro[4.5]decane-9-carboxylate (100 mg, 69.90%) as a yellow solid. LCMS:(ES·m/z):752[M+H]⁺.

### Step 2: Synthesis of Compound 3

To a stirred solution of tert-butyl 2-[(2-{6-[(2-cyanoethyl)amino]-4-{3-[(4-methyl-1,2,4-triazol-3-yl)methyl]oxetan-3-yl}pyridin-2-yl}-3-oxo-7-(trifluoromethyl)-1H-isoindol-5-yl)methyl]-6-oxa-2,9-diazaspiro[4.5]decane-9-carboxylate (100 mg, 0.13 mmol, 1 equiv) in DCM (2 mL) was added TFA (500 uL) dropwise at 0° C. under nitrogen atmosphere. The resulting mixture was stirred for additional 2 h at room temperature. LCMS indicated the reaction was completed. The resulting mixture was concentrated under reduced pressure. The crude product was used in the next step directly without further purification. LCMS:(ES·m/z):652[M+H]⁺.

### Step 3: Synthesis of Compound 163

To a stirred solution of 3-[(4-{3-[(4-methyl-1,2,4-triazol-3-yl)methyl]oxetan-3-yl}-6-(6-{6-oxa-2,9-diazaspiro[4.5]decan-2-ylmethyl}-1-oxo-4-(trifluoromethyl)-3H-isoindol-2-yl)pyridin-2-yl)amino]propanenitrile (60 mg, 0.09 mmol, 1 equiv) and TEA (27 mg, 0.27 mmol, 3 equiv) in DCM (1.2 mL) was added HCHO (8 mg, 0.27 mmol, 3 equiv) in portions at room temperature. The resulting mixture was stirred for 15 min at room temperature under air atmosphere. To the above mixture was added STAB (78 mg, 0.36 mmol, 4 equiv) in portions at room temperature. The resulting mixture was stirred for additional 2 h at room temperature. LCMS indicated the reaction was completed. The reaction was quenched by the addition of MeOH (1 mL) at 0° C. The resulting mixture was concentrated under reduced pressure. The residue was purified by reverse flash chromatography with the following conditions: column, C18 silica gel; mobile phase, ACN in water (0.05% TFA), 0% to 50% gradient in 30 min; detector, UV 254 nm and 220 nm. The crude product (25 mg) was purified by Prep-HPLC with the following conditions (Column: XBridge Shield RP18 OBD Column, 30*150 mm, 5 m; Mobile Phase A: Water (10 mmol/L NH₄HCO₃), Mobile Phase B: ACN; Flow rate: 60 mL/min; Gradient: 25% B to 50% B in 7 min, 50% B; Wave Length: 254 nm; RT1 (min): 5; The eluted was lyophilized to afford 3-[(4-{3-[(4-methyl-1,2,4-triazol-3-yl)methyl]oxetan-3-yl}-6-[6-({9-methyl-6-oxa-2,9-diazaspiro[4.5]decan-2-yl}methyl)-1-oxo-4-(trifluoromethyl)-3H-isoindol-2-yl]pyridin-2-yl)amino]propanenitrile (8.5 mg, 13.80%) as a white solid. LCMS:(ES·m/z):666[M+H]⁺. ¹H NMR (400 MHz, DMSO-d₆) δ 8.24 (s, 1H), 7.96-7.93 (m, 2H), 7.50 (s, 1H), 7.09-7.06 (m, 1H), 6.05 (s, 1H), 5.20 (s, 2H), 4.91 (d, J=8 Hz, 2H), 4.80 (d, J=8 Hz, 2H), 3.77 (s, 2H), 3.59-3.56 (m, 1H), 3.34-3.30 (m, 6H), 3.25 (s, 3H), 2.83-2.80 (m, 2H), 2.71-2.68 (m, 1H), 2.58-2.55 (m, 2H), 2.31-2.24 (m, 2H), 2.15-2.13 (m, 5H), 1.80-1.77 (m, 2H).

### Synthesis of Compound 164

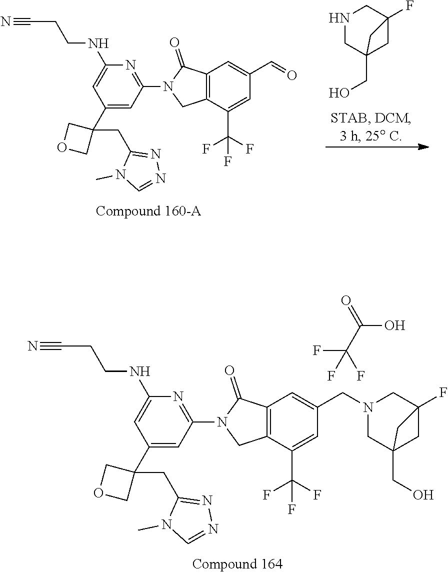

A mixture of 3-({6-[6-formyl-1-oxo-4-(trifluoromethyl)-3H-isoindol-2-yl]-4-{3-[(4-methyl-1,2,4-triazol-3-yl)methyl]oxetan-3-yl}pyridin-2-yl}amino)propanenitrile (70 mg, 0.13 mmol, 1 equiv), {5-fluoro-3-azabicyclo[3.1.1]heptan-1-yl}methanol (19 mg, 0.13 mmol, 1 equiv) and STAB (56 mg, 0.26 mmol, 2 equiv) in DCM (2 mL) was stirred for 3 h at 25° C. LCMS indicated the reaction was completed. The solvent was removed under reduced pressure. The residue was purified with prep-HPLC with following condition: Column: XBridge Prep C18 OBD Column, 19*150 mm, 5 m; Mobile Phase A: Water (0.05% TFA), Mobile Phase B: ACN; Flow rate: 25 mL/min; Gradient: 18% B to 48% B in 7 min, 48% B; Wave Length: 254 nm; RT1 (min): 5.08. The collected fraction was lyophilized to afford 3-{[6-(6-{[1-fluoro-5-(hydroxymethyl)-3-azabicyclo[3.1.1]heptan-3-yl]methyl}-1-oxo-4-(trifluoromethyl)-3H-isoindol-2-yl)-4-{3-[(4-methyl-1,2,4-triazol-3-yl)methyl]oxetan-3-yl}pyridin-2-yl]amino}propanenitrile; trifluoroacetic acid (23.3 mg, 21.94%) as a yellow solid. LCMS (ESI, m/z): 655[M+H−TFA]⁺; ¹H NMR (400 MHz, Methanol-d₄) δ 8.72 (s, 1H), 8.27 (s, 1H), 8.21 (s, 1H), 7.38 (d, J=1.2 Hz, 1H), 6.02 (d, J=1.2 Hz, 1H), 5.31 (s, 3H), 5.02 (dd, J=6.4, 2.4 Hz, 2H), 4.69 (s, 2H), 3.75 (s, 2H), 3.70-3.67 (m, 4H), 3.55 (s, 2H), 3.41 (s, 3H), 3.33 (s, 3H), 2.82 (t, J=6.4 Hz, 2H), 2.31-2.21 (m, 4H).

### Synthesis of Compound 165

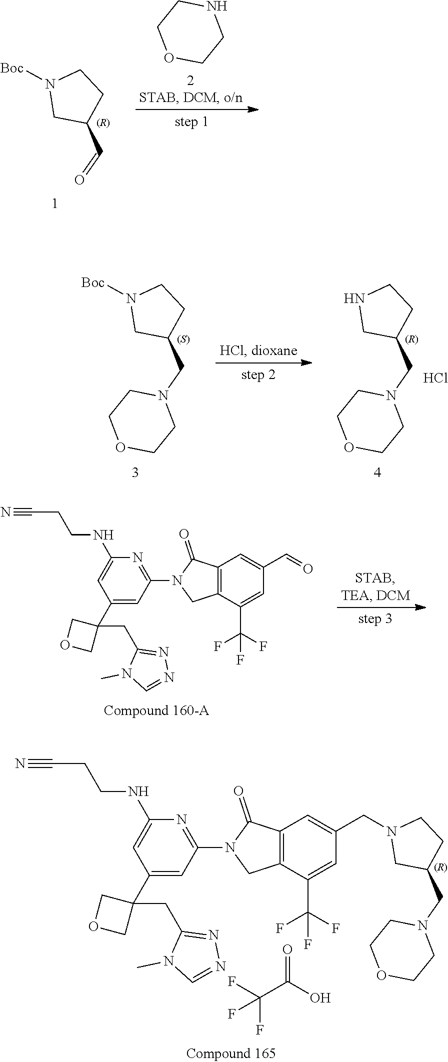

### Step 1: Synthesis of Compound 3

To a stirred solution of tert-butyl (3R)-3-formylpyrrolidine-1-carboxylate (500 mg, 2.50 mmol, 1 equiv) and morpholine (262 mg, 3.01 mmol, 1.2 equiv) in DCM (10 mL) was stirred for 30 min under nitrogen atmosphere. To the above mixture was added STAB (2.13 g, 10.03 mmol, 4 equiv) in portions at room temperature. The resulting mixture was stirred for additional overnight at room temperature. LCMS indicated the reaction was completed. The reaction was quenched with MeOH at 0° C. The resulting mixture was concentrated under reduced pressure. The residue was purified by silica gel column chromatography, eluted with PE/EA (1:1) to afford tert-butyl (3S)-3-(morpholin-4-ylmethyl)pyrrolidine-1-carboxylate (510 mg, 75%) as a white solid. LCMS (ESI, m/z):271[M+H]⁺, 215[M+H−56]⁺

### Step 2. Synthesis of Compound 4

A solution of tert-butyl (3S)-3-(morpholin-4-ylmethyl)pyrrolidine-1-carboxylate (500 mg, 1.84 mmol, 1 equiv) in HCl (4M in 1,4-dioxane, 5 mL) was stirred for 2 h at 0° C. under nitrogen atmosphere. LCMS indicated the reaction was completed. The resulting mixture was concentrated under reduced pressure. This resulted in 4-[(3R)-pyrrolidin-3-ylmethyl]morpholine hydrochloride (210 mg, 54%) as a white solid. LCMS (ESI, m/z):171[M+H]⁺

### Step 3: Synthesis of Compound 165

To a stirred solution of 3-({6-[6-formyl-1-oxo-4-(trifluoromethyl)-3H-isoindol-2-yl]-4-{3-[(4-methyl-1,2,4-triazol-3-yl)methyl]oxetan-3-yl}pyridin-2-yl}amino)propanenitrile (40 mg, 0.07 mmol, 1 equiv), TEA (15 mg, 0.15 mmol, 2 equiv) and 4-[(3R)-pyrrolidin-3-ylmethyl]morpholine (15 mg, 0.09 mmol, 1.2 equiv) in DCM (2 mL) stirred for 30 min at r.t. To the above mixture was added STAB (64 mg, 0.30 mmol, 4 equiv) in portions at room temperature. The resulting mixture was stirred for additional 1 h at room temperature. LCMS indicated the reaction was completed. The reaction was quenched by the addition of MeOH (10 mL) at 0° C. The resulting mixture was concentrated under reduced pressure. The residue was purified by reverse flash chromatography with the following conditions: column, C18 silica gel; mobile phase, ACN in water (0.05% TFA), 0% to 50% gradient in 30 min; detector, UV 254 nm and 220 nm. The crude product (30 mg) was purified by Prep-HPLC with the following conditions (Column: XSelect CSH Prep C18 OBD Column, 19*150 mm, 5 m; Mobile Phase A: Water (0.05% TFA), Mobile Phase B: ACN; Flow rate: 25 mL/min; Gradient: 5% B to 35% B in 7 min, 35% B; Wave Length: 254 nm; RT1 (min): 5.55; The collected fraction was lyophilized to afford 3-[(4-{3-[(4-methyl-1,2,4-triazol-3-yl)methyl]oxetan-3-yl}-6-(6-{[(3R)-3-(morpholin-4-ylmethyl)pyrrolidin-1-yl]methyl}-1-oxo-4-(trifluoromethyl)-3H-isoindol-2-yl)pyridin-2-yl)amino]propanenitrile; trifluoroacetic acid (16.2 mg, 26%) as a yellow solid. LCMS (ESI, m/z):680[M+H−TFA]⁺; ¹H NMR (300 MHz, DMSO-d₆) δ 10.94 (br s, 1H), 8.47 (s, 1H), 8.25 (d, J=11.1 Hz, 2H), 7.49 (s, 1H), 7.12 (br s, 1H), 6.12 (s, 1H), 5.26 (s, 2H), 4.92 (d, J=6.3 Hz, 2H), 4.80 (d, J=6.3 Hz, 2H), 4.64 (br s, 2H), 3.89-3.52 (m, 10H), 3.31-3.07 (m, 12H), 2.83 (t, J=6.3 Hz, 2H), 2.45-2.11 (m, 1H), 1.90-1.53 (m, 1H).

### Synthesis of Compound 166

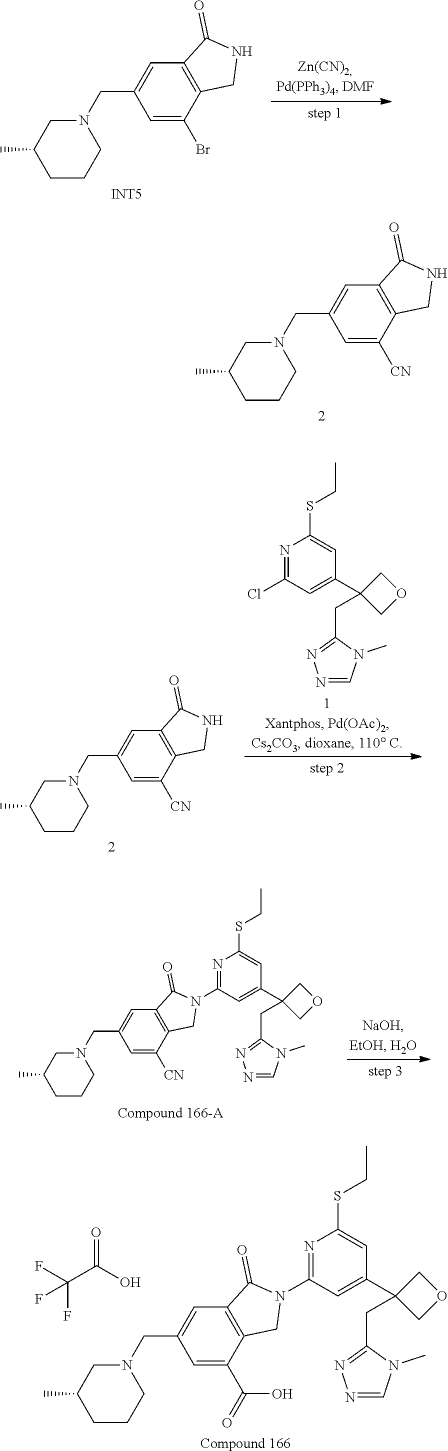

### Step 1: Synthesis of Compound 2

To a stirred solution of 4-bromo-6-{[(3S)-3-methylpiperidin-1-yl]methyl}-2,3-dihydroisoindol-1-one (200 mg, 0.62 mmol, 1 equiv) in DMF (2 mL) was added Zn(CN)₂ (145 mg, 1.23 mmol, 2 equiv) and Pd(PPh₃)₄ (71 mg, 0.06 mmol, 0.1 equiv) in portions at room temperature under N₂ atmosphere. The resulting mixture was stirred for 2 h at 145° C. under nitrogen atmosphere. The mixture was allowed to cool down to room temperature. LCMS indicated the reaction was 60% product. The reaction mixture was purified by reverse flash chromatography with the following conditions: column, C18 silica gel; mobile phase, TFA in Water (0.05% TFA), 0% to 50% gradient in 40 min; detector, UV 254 nm. This resulted in 6-{[(3S)-3-methylpiperidin-1-yl]methyl}-1-oxo-2,3-dihydroisoindole-4-carbonitrile (50 mg, 30%) as a white solid. LCMS:(ES·m/z):270[M+H]⁺.

### Step 2: Synthesis of Compound 166-A

To a stirred mixture of 6-{[(3S)-3-methylpiperidin-1-yl]methyl}-1-oxo-2,3-dihydroisoindole-4-carbonitrile (50 mg, 0.18 mmol, 1 equiv) and 2-chloro-6-(ethylsulfanyl)-4-{3-[(4-methyl-1,2,4-triazol-3-yl)methyl]oxetan-3-yl}pyridine (60 mg, 0.18 mmol, 1 equiv) in dioxane (1 mL) was added Cs₂CO₃ (120 mg, 0.37 mmol, 2 equiv) and Pd(OAc)₂ (4 mg, 0.02 mmol, 0.1 equiv) and Xantphos (21 mg, 0.04 mmol, 0.2 equiv) in portions at room temperature. The resulting mixture was stirred for 1.5 h at 110° C. under nitrogen atmosphere. The mixture was allowed to cool down to room temperature. LCMS indicated the reaction was completed. The resulting mixture was concentrated under reduced pressure. The residue was purified by reverse flash chromatography with the following conditions: column, C18 silica gel; mobile phase, TFA in Water (0.05% TFA), 0% to 50% gradient in 40 min; detector, UV 254 nm. The crude product (50 mg) was purified by Prep-HPLC with the following conditions (Column: XSelect CSH Prep C18 OBD Column, 19*250 mm, 5 m; Mobile Phase A: Water (0.05% TFA), Mobile Phase B: ACN; Flow rate: 25 mL/min; Gradient: 22% B to 42% B in 7 min, 42% B; Wave Length: 254 nm; RT1 (min): 5.38; The collected fraction was lyophilized to afford 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-1-oxo-3H-isoindole-4-carbonitrile; trifluoroacetic acid (47.5 mg, 37%) as a white solid. LCMS:(ES·m/z):558[M+H−TFA]⁺. ¹H NMR (400 MHz, DMSO-d₆) δ 9.65 (br s, 1H), 8.48-8.46 (m, 1H), 8.30 (s, 2H), 8.07 (s, 1H), 7.02 (s, 1H), 5.34 (s, 2H), 4.92 (d, J=8.0 Hz, 2H), 4.85 (d, J=8.0 Hz, 2H), 4.52-4.48 (m, 2H), 3.63 (s, 2H), 3.39-3.37 (m, 4H), 3.30-3.27 (m, 1H), 3.21-3.16 (m, 2H), 2.86-2.83 (m, 1H), 2.60-2.57 (m, 1H), 1.86-1.62 (m, 4H), 1.38 (t, J=7.2 Hz, 3H), 1.08-1.05 (m, 1H), 0.89 (d, J=6.4 Hz, 1H)

### Step 3: Synthesis of Compound 166

To a stirred solution of 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-1-oxo-3H-isoindole-4-carbonitrile (40 mg, 0.07 mmol, 1 equiv) in EtOH (1 mL) was added NaOH (28 mg, 0.72 mmol, 10 equiv) and H₂O (1 mL) dropwise at room temperature. The resulting mixture was stirred for overnight at 85° C. under air atmosphere. LCMS indicated the reaction was completed. The mixture was allowed to cool down to room temperature. The reaction mixture was concentrated to dryness under vacuum. The residue was purified by reverse flash chromatography with the following conditions: column, C18 silica gel; mobile phase, TFA in Water (0.05% TFA), 0% to 50% gradient in 40 min; detector, UV 254 nm. The collected fraction was concentrated to afford crude product (30 mg). The crude product (30 mg) was purified by Prep-HPLC with the following conditions (Column: XSelect CSH Prep C18 OBD Column, 19*150 mm, 5 m; Mobile Phase A: Water (0.05% TFA), Mobile Phase B: ACN; Flow rate: 25 mL/min; Gradient: 12% B to 26% B in 10 min, 26% B to 26% B in 11 min, 26% B; Wave Length: 254 nm; RT1 (min): 10.41; The eluted was lyophillized to afford 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-1-oxo-3H-isoindole-4-carboxylic acid; trifluoroacetic acid (3.3 mg, 6%) as a white solid. LCMS:(ES·m/z):577[M+H−TFA]⁺. ¹H NMR (400 MHz, DMSO-d₆) δ9.67 (br s, 1H), 8.40-8.38 (m, 2H), 8.20 (s, 1H), 8.07 (s, 1H), 6.97 (m, 1H), 5.37 (s, 2H), 4.95-4.82 (m, 4H), 4.64-4.43 (m, 2H), 3.61 (s, 2H), 3.51-3.36 (m, 4H), 3.29-3.26 (m, 1H), 3.18-3.13 (m, 2H), 2.86-2.84 (m, 1H), 2.60-2.55 (m, 1H), 1.86-1.59 (m, 4H), 1.41 (t, J=6.0 Hz, 3H), 1.10-1.04 (m, 1H), 0.95 (d, J=6.0 Hz, 3H).

### Synthesis of Compound 167

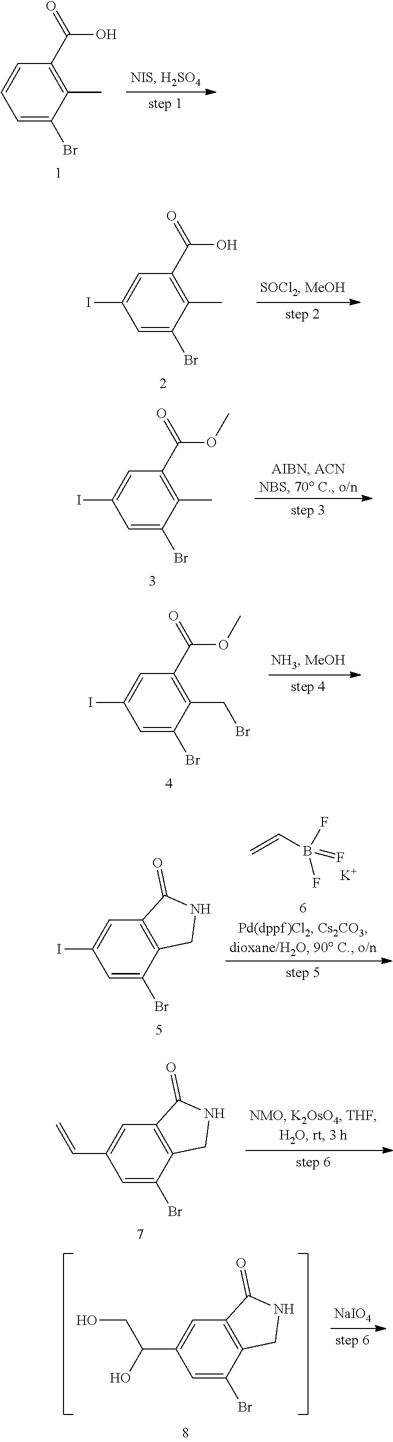

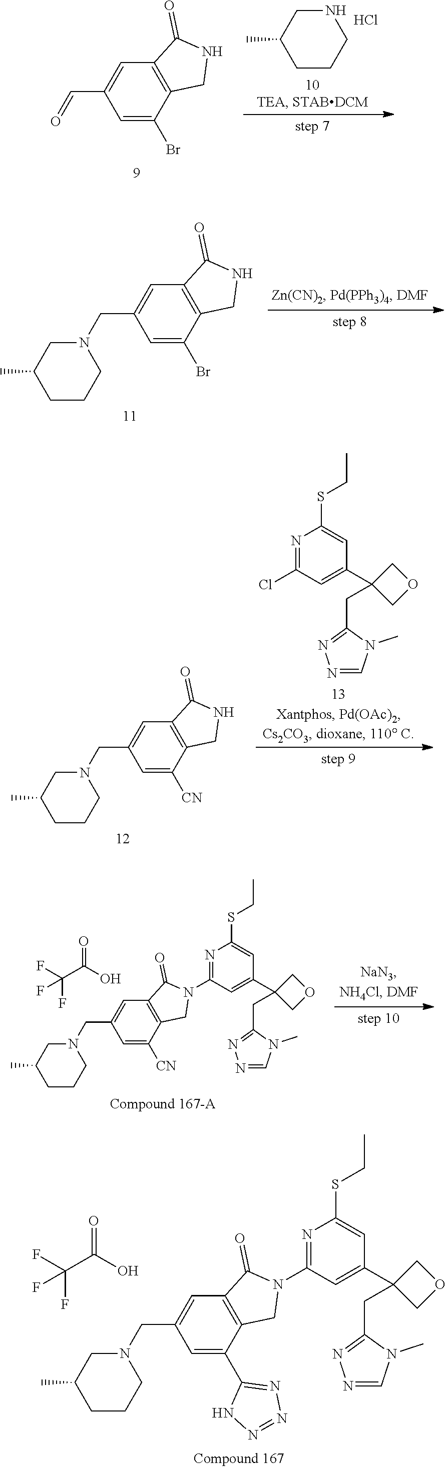

### Step 1: Synthesis of Compound 2

To a stirred solution of NIS (57.54 g, 255.75 mmol, 1.1 equiv) in H2SO₄ (200 mL) was stirred for 40 min at room temperature under nitrogen atmosphere. To the above mixture was added 3-bromo-2-methylbenzoic acid (50 g, 232.50 mmol, 1 equiv) in H2SO₄ (200 mL) dropwise at 0° C. and stirred for 3 h, The resulting mixture was stirred for additional overnight at room temperature. LCMS indicated the reaction was completed. The reaction was pour into Water/Ice (1000 mL) at 0° C. The precipitated solids were collected by filtration and washed with Et₂O (3×50 mL). The residue was purified by trituration with DCM (100 mL). The precipitated solids were collected by filtration and washed with DCM (3×50 mL). This resulted in 3-bromo-5-iodo-2-methylbenzoic acid (54.2 g, 68%) as an off-white solid. LCMS (ESI, ms):339[M−H]^{- 1}H NMR (300 MHz, DMSO-d₆) δ 8.13 (d, J=1.8 Hz, 1H), 7.99 (d, J=1.8 Hz, 1H), 2.47 (s, 3H).

### Step 2: Synthesis of Compound 3

To a stirred solution of 3-bromo-5-iodo-2-methylbenzoic acid (25 g, 73.32 mmol, 1 equiv) in MeOH (250 mL) was added SOCl₂ (43.61 g, 366.63 mmol, 5 equiv) dropwise at 0° C. under nitrogen atmosphere. The resulting mixture was stirred for 3 h at 70° C. under nitrogen atmosphere. TLC indicated the reaction was completed. The resulting mixture was allowed to cool to room temperature and concentrated under reduced pressure. This resulted in methyl 3-bromo-5-iodo-2-methylbenzoate (23 g, 88%) as off-white solid. ¹H NMR (400 MHz, Chloroform-d) δ 8.03 (d, J=2.0 Hz, 1H), 8.02 (d, J=2.0 Hz, 1H), 3.90 (s, 3H), 2.56 (s, 3H).

### Step 3. Synthesis of Compound 4

To a stirred solution of methyl 3-bromo-5-iodo-2-methylbenzoate (15 g, 42.25 mmol, 1 equiv) and NBS (9.03 g, 50.70 mmol, 1.2 equiv) in ACN (150 mL) was added AIBN (3.47 g, 21.12 mmol, 0.5 equiv) in portions at 70° C. under nitrogen atmosphere. LCMS & TLC showed the reaction was completed. no MS signal. The resulting mixture was concentrated under reduced pressure. The residue was purified by silica gel column chromatography, eluted with PE/EA (6:1) to afford methyl 3-bromo-2-(bromomethyl)-5-iodobenzoate (16.5 g, 90%) as a white solid. ¹H NMR (300 MHz, Chloroform-d) δ 8.20 (d, J=1.8 Hz, 1H), 8.11 (d, J=1.8 Hz, 1H), 5.06 (s, 2H), 1.73 (s, 3H).

### Step 4: Synthesis of Compound 5

To a stirred solution of methyl 3-bromo-5-iodo-2-methylbenzoate (14.5 g, 40.84 mmol, 1 equiv) in NH₃ (g, 7N in MeOH, 15 mL) was stirred for 1 h at room temperature under nitrogen atmosphere. LCMS indicated the reaction was completed. The resulting mixture was concentrated under vacuum. The residue was purified by trituration with water (50 mL). The precipitated solids were collected by filtration and washed with Et₂O (3×20 mL). This resulted in 4-bromo-6-iodo-2,3-dihydroisoindol-1-one (11.2 g, 81%) as a white solid. LCMS (ESI, ms):338,340[M+H]⁺

### Step 5: Synthesis of Compound 7

To a stirred solution of 4-bromo-6-iodo-2,3-dihydroisoindol-1-one (11.2 g, 33.14 mmol, 1 equiv) and potassium ethenyltrifluoroboranuide (3.55 g, 26.51 mmol, 0.8 equiv) in 1,4-dioxane (120 mL) and H₂O (12 mL) was added Cs₂CO₃ (21.60 g, 66.28 mmol, 2 equiv) and Pd(dppf)Cl₂ (2.43 g, 3.31 mmol, 0.1 equiv) at room temperature under nitrogen atmosphere. The resulting mixture was stirred for 1 h at 90° C. under nitrogen atmosphere. LCMS indicated the reaction was completed. The mixture was allowed to cool down to room temperature. The resulting mixture was concentrated under reduced pressure. The residue was purified by silica gel column chromatography, eluted with CH₂Cl₂/MeOH (12:1) to afford 4-bromo-6-ethenyl-2,3-dihydroisoindol-1-one (3.0 g, 38%) as a brown solid. LCMS (ESI, ms):238,240[M+H]⁺

### Step 6: Synthesis of Compound 9

To a stirred solution of 4-bromo-6-ethenyl-2,3-dihydroisoindol-1-one (5 g, 21.00 mmol, 1 equiv) and NMO (7.38 g, 63.00 mmol, 3 equiv) in THF (50 mL) and H₂O (25 mL) was added K₂OsO₄·2H₂O (0.77 g, 2.10 mmol, 0.1 equiv) at room temperature under nitrogen atmosphere. The resulting mixture was stirred for 3 h at room temperature under nitrogen atmosphere. To the above mixture was added NaIO₄ (22.46 g, 105.00 mmol, 5 equiv) at 0° C. The resulting mixture was stirred for additional 1 h at room temperature. LCMS indicated the reaction was completed. The reaction was quenched with Water at 0° C. Then EA (100 mL) was added. The precipitated solids were collected by filtration and washed with EtOAc (3×20 mL). This resulted in 7-bromo-3-oxo-1,2-dihydroisoindole-5-carbaldehyde (4 g, 79%) as a white solid. LCMS (ESI, ms):240,242[M+H]⁺

### Step 7: Synthesis of Compound 11

To a stirred solution of 7-bromo-3-oxo-1,2-dihydroisoindole-5-carbaldehyde (4 g, 16.66 mmol, 1 equiv) in DCM (80 mL) was added (3S)-3-methylpiperidine hydrochloride (2.71 g, 19.99 mmol, 1.2 equiv) and TEA (2.02 g, 19.99 mmol, 1.2 equiv) at room temperature under nitrogen atmosphere. The resulting mixture was stirred for 1 h at room temperature under nitrogen atmosphere. To the above mixture was added STAB (17.66 g, 83.31 mmol, 5 equiv) at room temperature. The resulting mixture was stirred for additional overnight at room temperature. LCMS indicated the reaction was completed. The resulting mixture was filtered, the filter cake was washed with CH₂Cl₂ (3×20 mL). The filtrate was concentrated under reduced pressure. The residue was purified by reverse flash chromatography with the following conditions: column, silica gel; mobile phase, MeCN in water (0.1% FA), 5% to 50% gradient in 30 min; detector, UV 254 nm. The collected fraction was lyophilized to afford 4-bromo-6-{[(3S)-3-methylpiperidin-1-yl]methyl}-2,3-dihydroisoindol-1-one (1.2 g, 22%) as a white solid. LCMS (ESI, ms):323, 325 [M+H]⁺

### Step 8: Synthesis of Compound 12

To a stirred solution of 4-bromo-6-{[(3S)-3-methylpiperidin-1-yl]methyl}-2,3-dihydroisoindol-1-one (200 mg, 0.62 mmol, 1 equiv) in DMF (2 mL) was added Zn(CN)₂ (145 mg, 1.23 mmol, 2 equiv) and Pd(PPh₃)₄ (71 mg, 0.06 mmol, 0.1 equiv) in portions at room temperature under N₂ atmosphere. The resulting mixture was stirred for 2 h at 145° C. under nitrogen atmosphere. The mixture was allowed to cool down to room temperature. LCMS indicated the reaction was 60% product. The reaction mixture was purified by reverse flash chromatography with the following conditions: column, C18 silica gel; mobile phase, TFA in Water (0.05% TFA), 0% to 50% gradient in 40 min; detector, UV 254 nm. This resulted in 6-{[(3S)-3-methylpiperidin-1-yl]methyl}-1-oxo-2,3-dihydroisoindole-4-carbonitrile (50 mg, 30%) as a white solid. LCMS:(ES·m/z):270[M+H]⁺.

### Step 9. Synthesis of Compound 167-A

To a stirred mixture of 6-{[(3S)-3-methylpiperidin-1-yl]methyl}-1-oxo-2,3-dihydroisoindole-4-carbonitrile (50 mg, 0.18 mmol, 1 equiv) and 2-chloro-6-(ethylsulfanyl)-4-{3-[(4-methyl-1,2,4-triazol-3-yl)methyl]oxetan-3-yl}pyridine (60 mg, 0.18 mmol, 1 equiv) in dioxane (1 mL) was added Cs₂CO₃ (120 mg, 0.37 mmol, 2 equiv) and Pd(OAc)₂ (4 mg, 0.02 mmol, 0.1 equiv) and Xantphos (21 mg, 0.04 mmol, 0.2 equiv) in portions at room temperature. The resulting mixture was stirred for 1.5 h at 110° C. under nitrogen atmosphere. The mixture was allowed to cool down to room temperature. LCMS indicated the reaction was completed. The resulting mixture was concentrated under reduced pressure. The residue was purified by reverse flash chromatography with the following conditions: column, C18 silica gel; mobile phase, TFA in Water (0.05% TFA), 0% to 50% gradient in 40 min; detector, UV 254 nm. The crude product (50 mg) was purified by Prep-HPLC with the following conditions (Column: XSelect CSH Prep C18 OBD Column, 19*250 mm, 5 m; Mobile Phase A: Water (0.05% TFA), Mobile Phase B: ACN; Flow rate: 25 mL/min; Gradient: 22% B to 42% B in 7 min. 42% B; Wave Length: 254 nm; RT1 (min): 5.38; The collected fraction was lyophilized to afford 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-1-oxo-3H-isoindole-4-carbonitrile; trifluoroacetic acid (47.5 mg, 37%) as a white solid. LCMS:(ES·m/z):558[M+H−TFA]⁺. ¹H NMR (400 MHz, DMSO-d₆) δ 9.65 (br s, 1H), 8.48-8.46 (m, 1H), 8.30 (s, 2H), 8.07 (s, 1H), 7.02 (s, 1H), 5.34 (s, 2H), 4.92 (d, J=8.0 Hz, 2H), 4.85 (d, J=8.0 Hz, 2H), 4.52-4.48 (m, 2H), 3.63 (s, 2H), 3.39-3.37 (m, 4H), 3.30-3.27 (m, 1H), 3.21-3.16 (m, 2H), 2.86-2.83 (m, 1H), 2.60-2.57 (m, 1H), 1.86-1.62 (m, 4H), 1.38 (t, J=7.2 Hz, 3H), 1.08-1.05 (m, 1H), 0.89 (d, J=6.4 Hz, 1H).

### Step 10: Synthesis of Compound 167

To a stirred solution of 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-1-oxo-3H-isoindole-4-carbonitrile (45 mg, 0.08 mmol, 1 equiv) in DMF (1 mL) was added NH₄Cl (43 mg, 0.81 mmol, 10 equiv) in portions at room temperature under air atmosphere. To the above mixture was added NaN₃ (52 mg, 0.81 mmol, 10 equiv) in portions at room temperature. The resulting mixture was stirred for additional 8 h at 120° C. LCMS indicated the reaction was completed. The mixture was allowed to cool down to room temperature. The residue was purified by reverse flash chromatography with the following conditions: column, C18 silica gel; mobile phase, TFA in Water (0.05% TFA), 0% to 50% gradient in 40 min; detector, UV 254 nm. The collected fraction was concentrated to afford crude product (25 mg). The crude product (25 mg) was purified by Prep-HPLC with the following conditions (Column: XSelect CSH Prep C18 OBD Column, 19*150 mm, 5 m; Mobile Phase A: Water (0.05% TFA), Mobile Phase B: ACN; Flow rate: 25 mL/min; Gradient: 13% B to 27% B in 10 min, 27% B to 27% B in 12 min, 27% B; Wave Length: 254 nm; RT1 (min): 10.83; The collected fraction was lyophilized to afford 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-4-(1H-1,2,3,4-tetrazol-5-yl)-3H-isoindol-1-one (6 mg, 12%) as a white solid. LCMS:(ES·m/z):601[M+1]. ¹H NMR (400 MHz, DMSO-d₆) δ 9.70 (br s, 1H), 8.54-8.48 (m, 1H), 8.17-8.10 (m, 2H), 6.99 (s, 1H), 5.48 (s, 2H), 4.93-4.85 (m, 4H), 4.58-4.51 (m, 2H), 3.64 (s, 2H), 3.51-3.33 (m, 5H), 3.23-3.17 (m, 2H), 2.89-2.80 (m, 1H), 2.67-2.63 (m, 1H), 1.86-1.64 (m, 4H), 1.43 (t, J=8.0 Hz, 3H), 1.09-1.03 (m, 1H), 0.90 (d, J=8.0 Hz, 3H)

### Synthesis of Compound 168

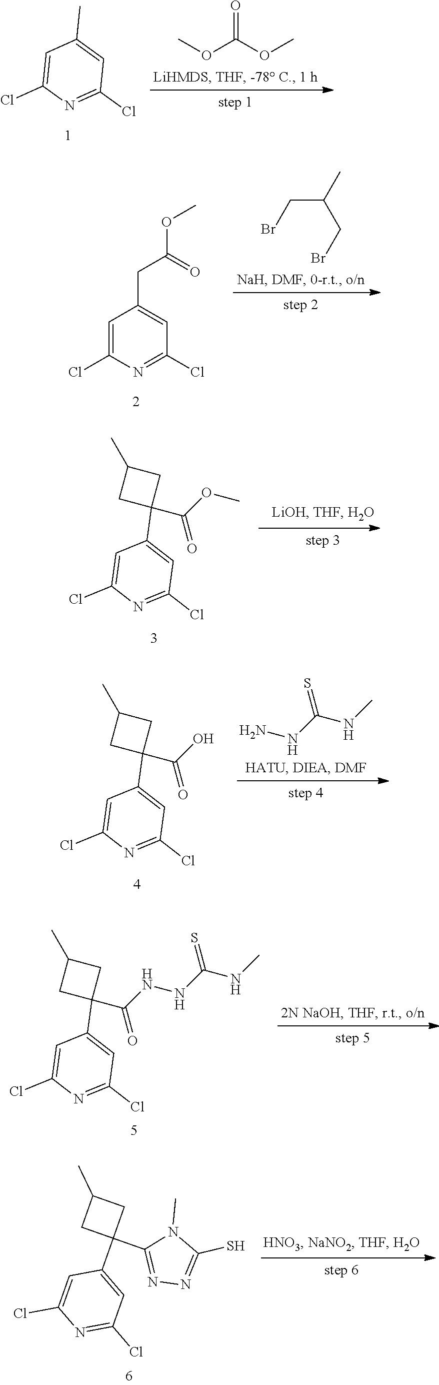

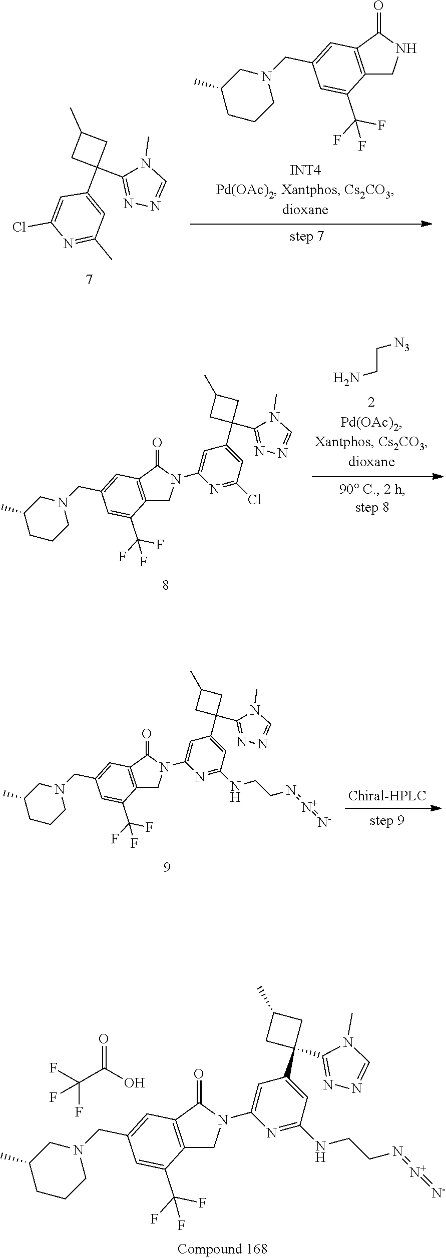

### Step 1: Synthesis of Compound 2

To a stirred mixture of 2,6-dichloro-4-methylpyridine (1.0 g, 6.17 mmol, 1.0 equiv) in THE (10.0 mL) were added LiHMDS (1N in THF, 8.02 mL) at −70° C. under nitrogen atmosphere, The reaction mixture was stirred for 30 min at −70 under nitrogen atmosphere. To the above mixture was added dimethyl carbonate (1.0 g, 11.10 mmol, 1.80 equiv) at −70° C. under nitrogen atmosphere. The resulting mixture was stirred for 2 h at −70° C. under nitrogen atmosphere. ˜50% desired product could be detected by LCMS. The reaction was quenched with sat. NH₄Cl (aq., 100 mL) at 0° C. The aqueous layer was extracted with CH₂Cl₂ (3×200 mL). The resulting mixture was concentrated under vacuum. The residue was purified by silica gel column chromatography, eluted with PE/EA (3:1) to afford methyl 2-(2,6-dichloropyridin-4-yl)acetate (590 mg, 41%) as an oil. LCMS (ES, m/z): 220,222 [M+H]⁺.

### Step 2: Synthesis of Compound 3

To a stirred solution of methyl 2-(2,6-dichloropyridin-4-yl)acetate (5.0 g, 22.72 mmol, 1.0 equiv) in DMF (50 mL) were added NaH (2.73 g, 68.16 mmol, 3.00 equiv, 60%) in portions at 0° C. under nitrogen atmosphere. The resulting mixture was stirred for 30 min at 0° C. under nitrogen atmosphere. To the above mixture was added 1,3-dibromo-2-methylpropane (7.36 g, 34.08 mmol, 1.5 equiv) at 0° C. The resulting mixture was stirred for additional 2 h at 10° C. ˜30% desired product could be detected by LCMS. The reaction was quenched with water (200 mL) at room temperature. The mixture was acidified to pH 6 with conc. HCl. The aqueous layer was extracted with CH₂Cl₂ (3×300 mL). The resulting mixture was concentrated under vacuum. The residue was purified by reverse flash chromatography with the following conditions: column, C18 silica gel; mobile phase, ACN in water (0.1% FA), 10% to 70% gradient in 30 min; detector, UV 254 nm. The eluting isomer was extracted with CH₂Cl₂ (3×300 mL). The resulting mixture was concentrated under vacuum to afford methyl 1-(2,6-dichloropyridin-4-yl)-3-methylcyclobutane-1-carboxylate (1.9 g, 15%) as a green oil. LCMS: (ES, m/s): 274,276 [M+H]⁺.

### Step 3: Synthesis of Compound 4

To a stirred solution of methyl 1-(2,6-dichloropyridin-4-yl)-3-methylcyclobutane-1-carboxylate (1.8 g, 6.56 mmol, 1.0 equiv) in THE (18 mL) were added LiGH (0.31 g, 13.13 mmol, 2.0 equiv) (H₂O=9.0 mL) in portions at room temperature under nitrogen atmosphere. The resulting mixture was stirred for 1 h at room temperature under nitrogen atmosphere. LCMS indicated the reaction was completed. The reaction mixture was adjusted to pH=5 with 1N HCL. The mixture was purified by reverse flash chromatography with the following conditions: column, C18 silica gel; mobile phase, ACN in water (0.1% FA), 10% to 50% gradient in 30 min; detector, UV 254 nm. The aqueous layer was extracted with CH₂Cl₂ (3×300 mL). The resulting mixture was concentrated under vacuum to afford 1-(2,6-dichloropyridin-4-yl)-3-methylcyclobutane-1-carboxylic acid (2.0 g, 83%) as a semi-solid. LCMS: (ES, m/s): 260,262 [M+H]⁺.

### Step 4: Synthesis of Compound 5

To a stirred solution of 1-(2,6-dichloropyridin-4-yl)-3-methylcyclobutane-1-carboxylic acid (2.4 g, 9.22 mmol, 1.0 equiv) in DMF (24 mL) were added HATU (5.26 g, 13.84 mmol, 1.5 equiv), 1-amino-3-methylthiourea (1.16 g, 11.07 mmol, 1.2 equiv) and DIEA (2.39 g, 18.45 mmol, 2.0 equiv) in portions at 0° C. under nitrogen atmosphere. The resulting mixture was stirred for 2 h at room temperature under nitrogen atmosphere. LCMS indicated the reaction was completed. The reaction was quenched with Water at room temperature. The aqueous layer was extracted with CH₂Cl₂ (3×200 mL). The resulting mixture was concentrated under vacuum. The residue was purified by silica gel column chromatography, eluted with PE/EA (2:3) to afford 1-(2,6-dichloropyridin-4-yl)-3-methyl-N-[(methylcarbamothioyl)amino]cyclobutane-1-carboxamide (2.5 g, 70%) as a white solid. LCMS: (ES, m/s): 347,349 [M+H]⁺.

### Step 5: Synthesis of Compound 6

To a stirred solution of 1-(2,6-dichloropyridin-4-yl)-3-methyl-N-[(methylcarbamothioyl)amino]cyclobutane-1-carboxamide (2.3 g, 6.62 mmol, 1.0 equiv) in THF (11 mL) were added NaOH (1.0 g, 25.00 mmol, 3.77 equiv) (H₂O=11 mL) in portions at 0° C. under nitrogen atmosphere. The resulting mixture was stirred for overnight at room temperature under nitrogen atmosphere. ˜73% desired product could be detected by LCMS. The mixture was acidified to pH 5 with conc. HCl. The aqueous layer was extracted with CH₂Cl₂ (3×200 mL). The resulting mixture was concentrated under vacuum. The residue was purified by silica gel column chromatography, eluted with PE/EA (1:1) to afford 5-[1-(2,6-dichloropyridin-4-yl)-3-methylcyclobutyl]-4-methyl-1,2,4-triazole-3-thiol (1.2 g, 55%) as a white solid. LCMS: (ES, m/s): 329,331 [M+H]⁺.

### Step 6: Synthesis of Compound 7

To a stirred solution of 5-[1-(2,6-dichloropyridin-4-yl)-3-methylcyclobutyl]-4-methyl-1,2,4-triazole-3-thiol (1.2 g, 3.64 mmol, 1.0 equiv) in THE (12 mL) were added NaNO₂ (0.75 g, 10.93 mmol, 3.0 equiv)(H₂O=12 mL) and HNO₃ (12 mL, 1.0 mol/L) in portions at 0° C. under nitrogen atmosphere. The resulting mixture was stirred for overnight at room temperature under nitrogen atmosphere. LCMS indicated the reaction was completed. The reaction was quenched with Water/Ice at room temperature. The aqueous layer was extracted with CH₂Cl₂ (3×200 mL). The resulting mixture was concentrated under vacuum. The residue was purified by reverse flash chromatography with the following conditions: column, silica gel; mobile phase ACN in water (0.05% TFA), 10% to 50% gradient in 30 min; detector, UV 254 nm. The aqueous layer was extracted with CH₂Cl₂ (3×200 mL). The resulting mixture was concentrated under vacuum to afford 2,6-dichloro-4-[3-methyl-1-(4-methyl-1,2,4-triazol-3-yl)cyclobutyl]pyridine (900 mg, 74%) as a green solid. LCMS: (ES, m/s): 297,299 [M+H]⁺.

### Step 7: Synthesis of Compound 8

To a stirred mixture of 2,6-dichloro-4-[3-methyl-1-(4-methyl-1,2,4-triazol-3-yl)cyclobutyl]pyridine (250 mg, 0.84 mmol, 1 equiv) and 6-{[(3S)-3-methylpiperidin-1-yl]methyl}-4-(trifluoromethyl)-2,3-dihydroisoindol-1-one (236 mg, 0.75 mmol, 0.9 equiv) in dioxane (5.00 mL) was added Cs₂CO₃ (548 mg, 1.68 mmol, 2 equiv) in portions and Pd(OAc)₂ (19 mg, 0.08 mmol, 0.1 equiv) and Xantphos (97 mg, 0.17 mmol, 0.2 equiv) at room temperature. The resulting mixture was stirred for 2 h at 90° C. under nitrogen atmosphere. The mixture was allowed to cool down to room temperature. LCMS indicated the reaction was 50% product. The resulting mixture was concentrated under reduced pressure. The residue was purified by reverse flash chromatography with the following conditions: column, C18 silica gel; mobile phase, TFA in Water (0.05% TFA), 0% to 50% gradient in 40 min; detector, UV 254 nm. The collected fraction was lyophilized to afford 2-{6-chloro-4-[3-methyl-1-(4-methyl-1,2,4-triazol-3-yl)cyclobutyl]pyridin-2-yl}-6-{[(3S)-3-methylpiperidin-1-yl]methyl}-4-(trifluoromethyl)-3H-isoindol-1-one (200 mg, 41%) as a yellow solid. LCMS:(ES·m/z):573,575[M+H]⁺.

### Step 8: Synthesis of Compound 9

To a stirred mixture of 2-{6-chloro-4-[3-methyl-1-(4-methyl-1,2,4-triazol-3-yl)cyclobutyl]pyridin-2-yl}-6-{[(3S)-3-methylpiperidin-1-yl]methyl}-4-(trifluoromethyl)-3H-isoindol-1-one (200 mg, 0.35 mmol, 1 equiv) and 2-azidoethanamine (36 mg, 0.42 mmol, 1.2 equiv) in dioxane (4 mL) was added Cs₂CO₃ (227 mg, 0.69 mmol, 2 equiv) in portions and Xantphos (40 mg, 0.07 mmol, 0.2 equiv) and Pd(OAc)₂ (8 mg, 0.03 mmol, 0.1 equiv) at room temperature. The resulting mixture was stirred for 1.5 h at 90° C. under nitrogen atmosphere. The mixture was allowed to cool down to room temperature. LCMS indicated the reaction was completed. The resulting mixture was concentrated under reduced pressure. The residue was purified by silica gel column chromatography, eluted with CH₂Cl₂/MeOH (10:1) to afford 2-{6-[(2-azidoethyl)amino]-4-[3-methyl-1-(4-methyl-1,2,4-triazol-3-yl)cyclobutyl]pyridin-2-yl}-6-{[(3S)-3-methylpiperidin-1-yl]methyl}-4-(trifluoromethyl)-3H-isoindol-1-one (90 mg, 41%) as a yellow solid. LCMS:(ES·m/z):623[M+H]⁺.

### Step 9: Synthesis of Compound 168

The 2-{6-[(2-azidoethyl)amino]-4-[3-methyl-1-(4-methyl-1,2,4-triazol-3-yl)cyclobutyl]pyridin-2-yl}-6-{[(3S)-3-methylpiperidin-1-yl]methyl}-4-(trifluoromethyl)-3H-isoindol-1-one (60 mg) was purified by Prep-HPLC with the following conditions (Column: XSelect CSH Prep C18 OBD Column, 19*250 mm, 5 m; Mobile Phase A: Water (0.05% TFA), Mobile Phase B: ACN; Flow rate: 25 mL/min; Gradient: 26% B to 41% B in 10 min, 41% B; Wave Length: 254 nm; RT1 (min): 8.08; The collected fraction was lyophilized to afford 45 mg pure product. The product was separated by Chiral-HPLC with the following conditions (Column: CHIRALPAK IE, 2*25 cm, 5 m; Mobile Phase A: Hex: DCM=3:1 (0.5% 2M NH₃-MeOH)--HPLC, Mobile Phase B: IPA--HPLC; Flow rate: 20 mL/min; Gradient: 10% B to 10% B in 33 min; Wave Length: 220/254 nm; RT1 (min): 25.218; RT2 (min): 30.073; Sample Solvent: EtOH--HPLC; Injection Volume: 0.6 mL; The first eluting isomer was concentrated and lyophilized to afford 2-{6-[(2-azidoethyl)amino]-4-[(1r,3s)-3-methyl-1-(4-methyl-1,2,4-triazol-3-yl)cyclobutyl]pyridin-2-yl}-6-{[(3S)-3-methylpiperidin-1-yl]methyl}-4-(trifluoromethyl)-3H-isoindol-1-one (30 mg, 50%). LCMS:(ES·m/z):623[M+H]⁺. ¹H NMR (400 MHz, DMSO-d₆) δ 8.35 (s, 1H), 7.95-7.91 (m, 2H), 7.76 (br s, 1H), 7.01 (br s, 1H), 6.20 (br s, 1H), 5.20 (s, 2H), 3.64-3.51 (m, 6H), 3.23 (s, 3H), 2.75-2.73 (m, 4H), 2.61-2.54 (m, 3H), 1.95-1.93 (m, 1H), 1.64-1.49 (m, 5H), 1.10 (d, J=8 Hz, 3H), 0.85-0.80 (m, 4H).

### Synthesis of Compound 169

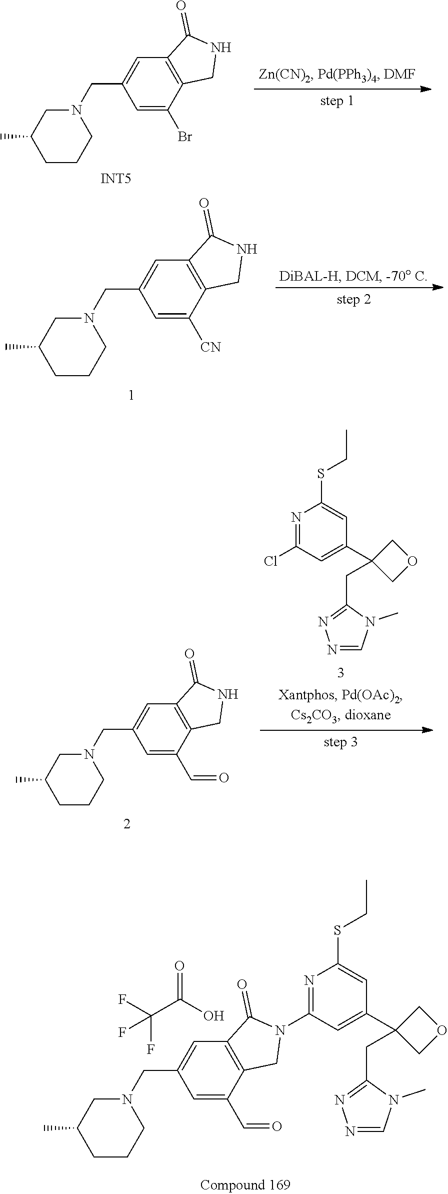

### Step 1: Synthesis of Compound 1

To a stirred solution of 4-bromo-6-{[(3S)-3-methylpiperidin-1-yl]methyl}-2,3-dihydroisoindol-1-one (200 mg, 0.62 mmol, 1 equiv) in DMF (2 mL) was added Zn(CN)₂ (145 mg, 1.23 mmol, 2 equiv) and Pd(PPh₃)₄ (71 mg, 0.06 mmol, 0.1 equiv) in portions at room temperature. The resulting mixture was stirred for 2 h at 145° C. under nitrogen atmosphere. LCMS indicated the reaction was 60% product. The mixture was allowed to cool down to room temperature. The reaction was concentrated to dryness under vacuum. The residue was purified by reverse flash chromatography with the following conditions: column, C18 silica gel; mobile phase, TFA in Water (0.05% TFA), 0% to 50% gradient in 40 min; detector, UV 254 nm. This resulted in 6-{[(3S)-3-methylpiperidin-1-yl]methyl}-1-oxo-2,3-dihydroisoindole-4-carbonitrile (50 mg, 30%) as a white solid. LCMS:(ES·m/z):270[M+H]⁺.

### Step 2: Synthesis of Compound 2

To a stirred solution of 6-{[(3S)-3-methylpiperidin-1-yl]methyl}-1-oxo-2,3-dihydroisoindole-4-carbonitrile (100 mg, 0.37 mmol, 1 equiv) in DCM (1 mL) were added DIBAL-H (1 mol/L n-hexane, 1.20 mL, 1.20 mmol, 3.3 equiv) dropwise at −70° C. under air atmosphere. The resulting mixture was stirred for additional 2 h at −70° C. LCMS indicated the reaction was 50% product. The reaction was quenched by the addition of MeOH (1 mL) at −10° C. The resulting mixture was concentrated under reduced pressure. The residue was dissolved in acetonitrile (2 mL). The residue was purified by reverse flash chromatography with the following conditions: column, C18 silica gel; mobile phase, ACN in Water (10 mmol/L NH₄HCO₃), 0% to 60% gradient in 30 min; detector, UV 254 nm. This resulted in 6-{[(3S)-3-methylpiperidin-1-yl]methyl}-1-oxo-2,3-dihydroisoindole-4-carbaldehyde (45 mg, 44%) as a white solid. LCMS:(ES·m/z):273[M+H]⁺.

### Step 3: Synthesis of Compound 3

A mixture of 2,6-dichloro-4-{3-[(4-methyl-1,2,4-triazol-3-yl)methyl]oxetan-3-yl}pyridine (300 mg, 1.00 mmol, 1 equiv) and (ethylsulfanyl)sodium (92 mg, 1.10 mmol, 1.1 equiv) in DMF (5 mL) was stirred for 3 h at 50° C. LCMS indicated the reaction was completed. The mixture was allowed to cool down to room temperature. The reaction was quenched by the addition of water (10 mL) at room temperature. The resulting mixture was extracted with EtOAc (3×10 mL). The combined organic layers were washed with brine (3×10 mL), dried over anhydrous Na₂SO₄. After filtration, the filtrate was concentrated under reduced pressure. The residue was purified by silica gel column chromatography, eluted with CH₂Cl₂/MeOH (10:1) to afford 2-chloro-6-(ethylsulfanyl)-4-{3-[(4-methyl-1,2,4-triazol-3-yl)methyl]oxetan-3-yl}pyridine (190 mg, 58%) as a yellow solid. LCMS (ES, m/z): 325 [M+H]⁺.

### Step 4: Synthesis of Compound 169

To a stirred mixture of 6-{[(3S)-3-methylpiperidin-1-yl]methyl}-1-oxo-2,3-dihydroisoindole-4-carbaldehyde (45 mg, 0.16 mmol, 1 equiv) and 2-chloro-6-(ethylsulfanyl)-4-{3-[(4-methyl-1,2,4-triazol-3-yl)methyl]oxetan-3-yl}pyridine (48 mg, 0.15 mmol, 0.9 equiv) in dioxane (1 mL) was added Cs₂CO₃ (107 mg, 0.33 mmol, 2 equiv) and Xantphos (19 mg, 0.03 mmol, 0.2 equiv) and Pd(OAc)₂ (4 mg, 0.01 mmol, 0.1 equiv) in portions at room temperature. The resulting mixture was stirred for 2 h at 110° C. under nitrogen atmosphere. The mixture was allowed to cool down to room temperature. LCMS indicated the reaction was completed. The resulting mixture was concentrated under reduced pressure. The residue was purified by silica gel column chromatography, eluted with CH₂Cl₂/MeOH (10:1) to afford the crude product (40 mg). The crude product (40 mg) was purified by Prep-HPLC with the following conditions (Column: XSelect CSH Prep C18 OBD Column, 19*250 mm, 5 m; Mobile Phase A: Water (0.05% TFA), Mobile Phase B: ACN; Flow rate: 25 mL/min; Gradient: 20% B to 44% B in 7 min, 44% B; Wave Length: 254 nm; RT1 (min): 5.4; The eluted was lyophilized to afford 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-1-oxo-3H-isoindole-4-carbaldehyde (28.3 mg, 30%) as a white solid. LCMS:(ES·m/z):561[M+H]⁺. ¹H NMR (400 MHz, DMSO-d₆) δ10.26 (s, 1H), 9.71 (br s, 1H), 8.56 (s, 1H), 8.38 (s, 1H), 8.28 (s, 1H), 8.09 (s, 1H), 7.00 (s, 1H), 5.43 (s, 2H), 4.92-4.84 (m, 4H), 4.58-4.51 (m, 2H), 3.65 (s, 2H), 3.43-3.40 (m, 4H), 3.31-3.29 (m, 1H), 3.22-3.17 (m, 2H), 2.89-2.86 (m, 1H), 2.62-2.57 (m, 1H), 1.86-1.63 (m, 4H), 1.40 (t, J=7.2 Hz, 3H), 1.08-1.04 (m, 1H), 0.89 (d, J=8.0 Hz, 3H)

### Synthesis of Compound 170

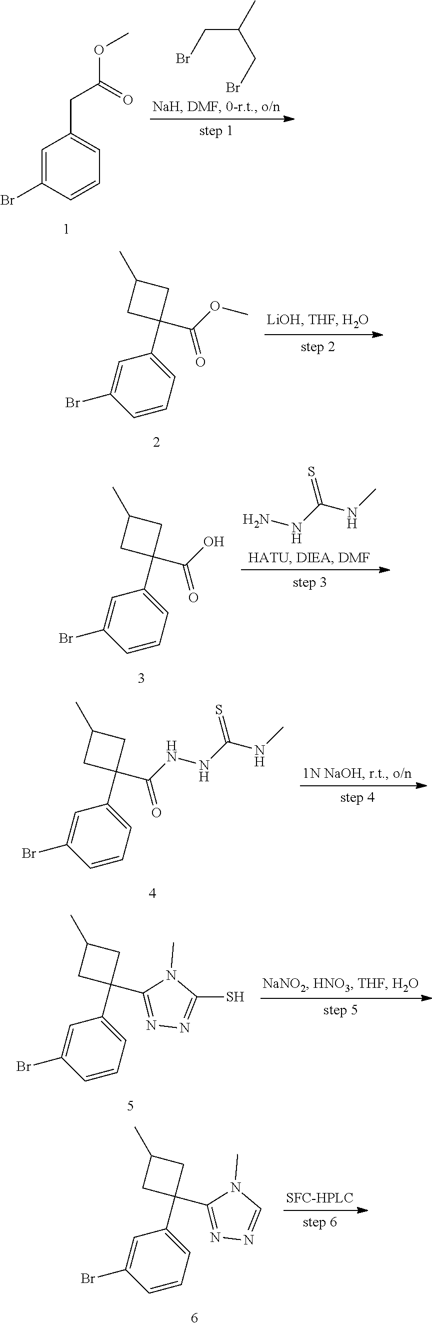

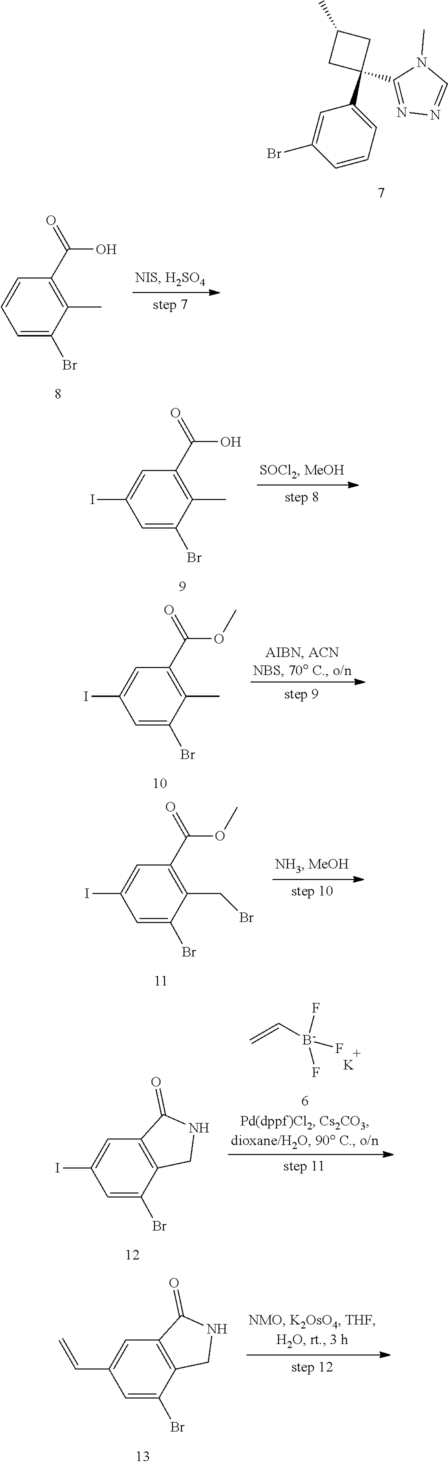

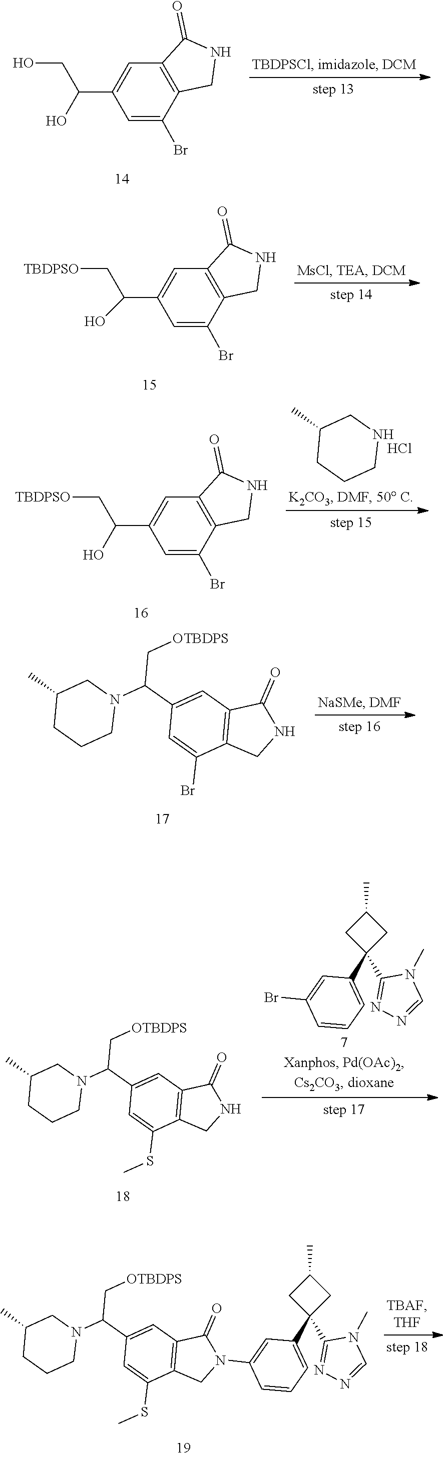

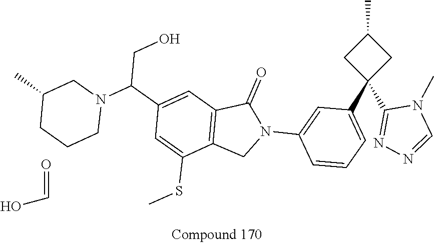

### Step 1: Synthesis of Compound 2

To a stirred solution of methyl 2-(3-bromophenyl)acetate (100 g, 436.54 mmol, 1 equiv) in DMF (1 L) was added NaH (26.19 g, 1091.35 mmol, 1.5 equiv, 60%) in portions at 0° C. under air atmosphere. The resulting mixture was stirred for 30 min at 0° C. under air atmosphere. To the above mixture was added 1,3-dibromo-2-methylpropane (103.68 g, 480.19 mmol, 1.1 equiv) in portions over at 0° C. The resulting mixture was stirred for additional overnight at room temperature. LCMS indicated the reaction was completed. The reaction was quenched by the addition of sat. NH₄Cl (aq.) (1000 mL) at 0° C. The resulting mixture was extracted with EtOAc (3×700 mL). The combined organic layers were washed with brine (3×200 mL), dried over anhydrous Na₂SO₄. After filtration, the filtrate was concentrated under reduced pressure. The residue was purified by silica gel column chromatography, eluted with PE/EA (5:1) to afford methyl 1-(3-bromophenyl)-3-methylcyclobutane-1-carboxylate (100 g, 81%) as a yellow oil. LCMS:(ES·m/z):283,284[M+H]⁺.

### Step 2: Synthesis of Compound 3

To a stirred solution of methyl 1-(3-bromophenyl)-3-methylcyclobutane-1-carboxylate (100 g, 353.15 mmol, 1 equiv) in THE (100 mL) was added LiGH (25.37 g, 1059.45 mmol, 3 equiv) in H₂O (1000 mL) at 0° C. The resulting mixture was stirred for overnight at room temperature under nitrogen atmosphere. LCMS indicated the reaction was completed. The resulting mixture was concentrated under vacuum. The mixture was acidified to pH 6 with conc. HCl. The resulting mixture was extracted with EtOAc (3×200 mL). The combined organic layers were washed with brine (3×100 mL), dried over anhydrous Na₂SO₄. After filtration, the filtrate was concentrated under reduced pressure. This resulted in 1-(3-bromophenyl)-3-methylcyclobutane-1-carboxylic acid (30 g, 32%) as a yellow solid. LCMS (ESI, m/z):267,269[M−H]⁻.

### Step 3: Synthesis of Compound 4

To a stirred solution of 1-(3-bromophenyl)-3-methylcyclobutane-1-carboxylic acid (16.5 g, 61.31 mmol, 1 equiv) and 1-amino-3-methylthiourea (7.74 g, 73.57 mmol, 1.2 equiv) in DMF (170 mL) was added HATU (34.97 g, 91.96 mmol, 1.5 equiv) and DIEA (15.85 g, 122.61 mmol, 2 equiv) at room temperature. The resulting mixture was stirred for overnight at room temperature under nitrogen atmosphere. LCMS indicated the reaction was completed. The reaction mixture was used to next step without any treatment. LCMS:(ES·m/z):356,358[M+H]⁺.

### Step 4: Synthesis of Compound 5

To the above mixture was added NaOH (170 mL) at room temperature. The resulting mixture was stirred for additional overnight at room temperature. LCMS indicated the reaction was completed. The mixture was acidified to pH 6 with 1N HCl. The resulting mixture was extracted with EtOAc (3×100 mL). The combined organic layers were washed with brine (5×100 mL), dried over anhydrous Na₂SO₄. After filtration, the filtrate was concentrated under reduced pressure. The residue was purified by silica gel column chromatography, eluted with CH₂Cl₂/MeOH (10:1) to afford 5-[1-(3-bromophenyl)-3-methylcyclobutyl]-4-methyl-1,2,4-triazole-3-thiol (4.1 g, 20%) as a yellow oil. LCMS:(ES·m/z):338,340[M+H]⁺.

### Step 5: Synthesis of Compound 6

To a stirred mixture of 5-[1-(3-bromophenyl)-3-methylcyclobutyl]-4-methyl-1,2,4-triazole-3-thiol (22 g, 65.03 mmol, 1.00 equiv) in THE (22.02 mL) was added NaNO₂ (22.44 g, 325.18 mmol, 5 equiv) in portions at 0 degrees C. under nitrogen atmosphere. The mixture was added HNO₃ (325.18 mL, 325.18 mmol, 5 equiv, 1N) dropwise at 0 degrees C. under nitrogen atmosphere. LCMS indicated the reaction was completed. The mixture was basified to pH 8 with saturated NaHCO₃ (aq.). The resulting mixture was extracted with EtOAc (3×200 mL). The combined organic layers were washed with water (4×80 mL). The resulting mixture was concentrated under reduced pressure. The residue was purified by silica gel column chromatography, eluted with CH₂Cl₂/MeOH (10:1) to afford 3-[1-(3-bromophenyl)-3-methylcyclobutyl]-4-methyl-1,2,4-triazole (10 g, 50%) as an off-white solid. LCMS:(ES·m/z):306,308[M+H]⁺.

### Step 6: Synthesis of Compound 7

The product (10 g) was separated by Prep-SFC with the following conditions (Column: CHIRALPAK ID 2*25 cm, Sum; Mobile Phase A: CO₂, Mobile Phase B: IPA (1%-2M-NH₃-MeOH); Flow rate: 55 mL/min; Gradient: isocratic 20% B; Column Temperature (° C.): 35; Back Pressure (bar): 100; Wave Length: 220 nm; RT1 (min): 12.13; RT2 (min): 13.8; Sample Solvent: MEOH; Injection Volume: 0.8 mL. The second fraction (RT13.8 min) was concentrated to afford 4-methyl-3-[(1r,3s)-1-(3-bromophenyl)-3-methylcyclobutyl]-1,2,4-triazole (6 g, 60%) as an off-white solid. ¹H NMR (300 MHz, Chloroform-d) δ 8.00 (s, 1H), 7.53 (s, 1H), 7.40-7.37 (s, 1H), 7.24-7.21 (m, 2H), 3.19 (s, 3H), 2.82-2.78 (m, 2H), 2.67-2.63 (m, 3H), 1.22-1.12 (m, 3H).

### Step 7. Synthesis of Compound 9

To a stirred solution of NIS (57.54 g, 255.76 mmol, 1.1 equiv) in H2SO₄ (200 mL) was stirred for 40 min at room temperature under nitrogen atmosphere. To the above mixture was added 3-bromo-2-methylbenzoic acid (50 g, 232.51 mmol, 1 equiv) in H2SO₄ (200 mL) dropwise at 0° C. and stirred for 3 h, The resulting mixture was stirred for additional overnight at room temperature. LCMS indicated complete reaction. LCMS indicated complete reaction. The reaction was pour into Water/Ice at 0° C. The precipitated solids were collected by filtration and washed with Et₂O (3×20 mL). The residue was purified by trituration with DCM (100 mL). The precipitated solids were collected by filtration and washed with DCM (3×50 mL). The solid was dried under infrared to afford 3-bromo-5-iodo-2-methylbenzoic acid (54.2 g, 68%) as an off-white solid. LCMS (ESI, ms):339,341[M−H]⁻. ¹H NMR (300 MHz, DMSO-d₆) δ 8.13 (d, J=1.8 Hz, 1H), 7.99 (d, J=1.8 Hz, 1H), 2.47 (s, 3H)

### Step 8: Synthesis of Compound 10

To a stirred solution of 3-bromo-5-iodo-2-methylbenzoic acid (30 g, 87.99 mmol, 1 equiv) in MeOH (300 mL) was added SOCl₂ (52.34 g, 439.96 mmol, 5 equiv) dropwise at 0° C. under nitrogen atmosphere. The resulting mixture was stirred for 3 h at 70° C. under nitrogen atmosphere. LCMS indicated the reaction was completed. The resulting mixture was concentrated under reduced pressure. The reaction was quenched with sat. NaHCO₃ (aq.) at 0° C. The resulting mixture was extracted with EtOAc (3×100 mL). The combined organic layers were washed with brine (100 mL), dried over anhydrous Na₂SO₄. After filtration, the filtrate was concentrated under reduced pressure. This resulted in methyl 3-bromo-5-iodo-2-methylbenzoate (29.9 g, 96%) as an off-white solid. No MS signal. ¹H NMR (400 MHz, Chloroform-d) δ 8.03 (d, J=1.9 Hz, 1H), 8.02 (d, J=1.9 Hz, 1H), 3.90 (s, 3H), 2.56 (s, 3H).

### Step 9: Synthesis of Compound 11

To a stirred solution of methyl 3-bromo-5-iodo-2-methylbenzoate (15 g, 42.25 mmol, 1 equiv) and NBS (9.03 g, 50.70 mmol, 1.2 equiv) in ACN (150 mL) was added AIBN (3.47 g, 21.12 mmol, 0.5 equiv) in portions at 70° C. under nitrogen atmosphere. LCMS & TLC showed the reaction was completed. no MS signal. The resulting mixture was concentrated under reduced pressure. The residue was purified by silica gel column chromatography, eluted with PE/EA (6:1) to afford methyl 3-bromo-2-(bromomethyl)-5-iodobenzoate (16.5 g, 90%) as a white solid. ¹H NMR (300 MHz, Chloroform-d) δ 8.20 (d, J=1.9 Hz, 1H), 8.11 (d, J=1.8 Hz, 1H), 5.06 (s, 2H), 1.73 (s, 3H).

### Step 10: Synthesis of Compound 12

A mixture of methyl 3-bromo-2-(bromomethyl)-5-iodobenzoate (51.5 g, 118.70 mmol, 1 equiv) and NH₃ (7N in MeOH, 500 mL) was stirred for 2 h at 25° C. LCMS indicated the reaction was completed. The solvent was removed under reduced pressure. The residue was used for next step without further purification. LCMS (ESI, ms): 338,340 [M+H]⁺.

### Step 11: Synthesis of Compound 13

To a stirred solution of 4-bromo-6-iodo-2,3-dihydroisoindol-1-one (48 g, 142.03 mmol, 1 equiv) and potassium ethenyltrifluoroboranuide (15.22 g, 113.62 mmol, 0.8 equiv) in 1,4-dioxane (500 mL) and H₂O (100 mL) was added Cs₂CO₃ (92.56 g, 284.07 mmol, 2 equiv) and Pd(dppf)Cl₂ (10.39 g, 14.20 mmol, 0.1 equiv) at room temperature under nitrogen atmosphere. The resulting mixture was stirred for 1 h at 90° C. under nitrogen atmosphere. LCMS indicated the reaction was completed. The mixture was allowed to cool down to room temperature. The resulting mixture was concentrated under reduced pressure. The residue was purified by silica gel column chromatography, eluted with CH₂Cl₂/MeOH (12:1) to afford 4-bromo-6-ethenyl-2,3-dihydroisoindol-1-one (11 g, 32%) as a brown solid. LCMS (ESI, ms):238,240[M+H]⁺.

### Step 12: Synthesis of Compound 14

To a stirred solution of 4-bromo-6-ethenyl-2,3-dihydroisoindol-1-one (20 g, 84.00 mmol, 1 equiv) and NMO (29.52 g, 252.00 mmol, 3.00 equiv) in THE (200 mL) and water (100 mL) was added K₂OsO₄·2H₂O (0.15 g, 0.42 mmol, 0.05 equiv) at room temperature under nitrogen atmosphere. The resulting mixture was stirred for 3 h at room temperature under nitrogen atmosphere. LCMS indicated the reaction was completed. THE was removed under reduced pressure. The residue was purified by reverse flash chromatography with the following conditions: column, C18 silica gel, 80 g, 20-35 um; mobile phase, water with 0.1% TFA and ACN (0% to 100% gradient in 50 min); detector, UV 254 nm. The collected fraction was lyophilized to afford 4-bromo-6-(1,2-dihydroxyethyl)-2,3-dihydroisoindol-1-one (6.7 g, 29%) as a brown solid. LCMS (ESI, ms): 272,274 [M+H]⁺.

### Step 13. Synthesis of Compound 15

To a stirred solution of 4-bromo-6-(1,2-dihydroxyethyl)-2,3-dihydroisoindol-1-one (5 g, 18.37 mmol, 1 equiv) in DCM (50 mL) was added imidazole (2.50 g, 36.75 mmol, 2 equiv) and TBDPSCl (5.05 g, 18.37 mmol, 1 equiv) dropwise at room temperature under air atmosphere. The resulting mixture was stirred for 2 h at room temperature under air atmosphere. LCMS indicated the reaction was completed. The resulting mixture was concentrated under reduced pressure. The residue was purified by silica gel column chromatography, eluted with PE/EA (1:1) to afford 4-bromo-6-{2-[(tert-butyldiphenylsilyl)oxy]-1-hydroxyethyl}-2,3-dihydroisoindol-1-one (3.6 g, 38%) as a white solid. LCMS:(ES·m/z):510,512[M+H]⁺.

### Step 14: Synthesis of Compound 16

To a stirred solution of 4-bromo-6-{2-[(tert-butyldiphenylsilyl)oxy]-1-hydroxyethyl}-2,3-dihydroisoindol-1-one (3.5 g, 6.85 mmol, 1 equiv) in DCM (70 mL) was added MsCl (0.94 g, 8.22 mmol, 1.2 equiv) and TEA (2.08 g, 20.56 mmol, 3 equiv) dropwise at room temperature under air atmosphere. The resulting mixture was stirred for 2 h at room temperature under air atmosphere. LCMS indicated the reaction was completed. The resulting mixture was concentrated under reduced pressure. The resulting mixture was used in the next step directly without further purification. LCMS:(ES·m/z):588,590[M+H]⁺.

### Step 15: Synthesis of Compound 17

To a stirred solution of 1-(7-bromo-3-oxo-1,2-dihydroisoindol-5-yl)-2-[(tert-butyldiphenylsilyl)oxy]ethyl methanesulfonate (3.5 g, 5.94 mmol, 1 equiv) and (3S)-3-methylpiperidine (0.71 g, 7.13 mmol, 1.2 equiv) in DMF (40 mL) was added K₂CO₃ (2.05 g, 14.86 mmol, 2.5 equiv) in portions at room temperature under air atmosphere. The resulting mixture was stirred for overnight at 50° C. under air atmosphere. LCMS indicated the reaction was completed. The mixture was allowed to cool down to room temperature. The reaction was quenched by the addition of Water/Ice (200 mL) at 0° C. The resulting mixture was extracted with EtOAc (3×100 mL). The combined organic layers were washed with brine (3×5 mL), dried over anhydrous Na₂SO₄. After filtration, the filtrate was concentrated under reduced pressure. The residue was purified by silica gel column chromatography, eluted with PE/EA (1:1) to afford 4-bromo-6-{2-[(tert-butyldiphenylsilyl)oxy]-1-[(3S)-3-methylpiperidin-1-yl]ethyl}-2,3-dihydroisoindol-1-one (2.8 g, 79%) as a yellow solid. LCMS:(ES·m/z):591,593[M+H]⁺.

### Step 16: Synthesis of Compound 18

To a stirred solution of 4-bromo-6-{2-[(tert-butyldiphenylsilyl)oxy]-1-[(3S)-3-methylpiperidin-1-yl]ethyl}-2,3-dihydroisoindol-1-one (2 g, 3.38 mmol, 1 equiv) in DMF (20 mL) was added (methylsulfanyl)sodium (0.36 g, 5.07 mmol, 1.5 equiv) in portions at room temperature under air atmosphere. The resulting mixture was stirred for 2 h at 100° C. LCMS indicated the reaction was completed. The mixture was allowed to cool down to room temperature. The residue was purified by reversed-phase flash chromatography with the following conditions: column, C18 silica gel; mobile phase, MeCN in Water (0.05% TFA), 0% to 50% gradient in 40 min; detector, UV 254 nm. The collected fraction was concentrated to afford 6-{2-[(tert-butyldiphenylsilyl)oxy]-1-[(3S)-3-methylpiperidin-1-yl]ethyl}-4-(methylsulfanyl)-2,3-dihydroisoindol-1-one (600 mg, 32%) as a yellow oil. LCMS:(ES·m/z):559[M+H].

### Step 17: Synthesis of Compound 19

To a stirred mixture of 6-{2-[(tert-butyldiphenylsilyl)oxy]-1-[(3S)-3-methylpiperidin-1-yl]ethyl}-4-(methylsulfanyl)-2,3-dihydroisoindol-1-one (600 mg, 1.074 mmol, 1 equiv) and 4-methyl-3-[(1r,3s)-1-(3-bromophenyl)-3-methylcyclobutyl]-1,2,4-triazole (394 mg, 1.28 mmol, 1.2 equiv) in dioxane (12 mL) was added Cs₂CO₃ (699 mg, 2.14 mmol, 2 equiv) and Xantphos (124 mg, 0.21 mmol, 0.2 equiv) and Pd(OAc)₂ (24 mg, 0.10 mmol, 0.1 equiv) in portions at room temperature under nitrogen atmosphere. The resulting mixture was stirred for 2 h at 110° C. under nitrogen atmosphere. LCMS indicated the reaction was 45% product. The mixture was allowed to cool down to room temperature. The resulting mixture was concentrated under reduced pressure. The residue was purified by silica gel column chromatography, eluted with CH₂Cl₂/MeOH (10:1) to afford 6-{2-[(tert-butyldiphenylsilyl)oxy]-1-[(3S)-3-methylpiperidin-1-yl]ethyl}-4-(methylsulfanyl)-2-{3-[(1r,3s)-3-methyl-1-(4-methyl-1,2,4-triazol-3-yl)cyclobutyl]phenyl}-3H-isoindol-1-one (270 mg, 32%) as a yellow solid. LCMS:(ES·m/z):784[M+H]⁺.

### Step 18: Synthesis of Compound 170

To a stirred solution of 6-{2-[(tert-butyldiphenylsilyl)oxy]-1-[(3S)-3-methylpiperidin-1-yl]ethyl}-4-(methylsulfanyl)-2-{3-[(1r,3s)-3-methyl-1-(4-methyl-1,2,4-triazol-3-yl)cyclobutyl]phenyl}-3H-isoindol-1-one (200 mg, 0.25 mmol, 1 equiv) in THE (4 mL) was added TBAF (133 mg, 0.51 mmol, 2 equiv) in portions at 0° C. The resulting mixture was stirred for 2 h at room temperature under air atmosphere. LCMS indicated the reaction was 30% product. The resulting mixture was concentrated under reduced pressure. The crude product (40 mg) was purified by Prep-HPLC with the following conditions (Column: XSelect CSH Fluoro Phenyl, 30*150 mm, 5 m; Mobile Phase A: Water (0.1% FA), Mobile Phase B: ACN; Flow rate: 60 mL/min; Gradient: 5% B to 35% B in 8 min, 35% B; Wave Length: 254; 220 nm; RT1 (min): 7.9; The collected fraction was lyophilized to afford 6-{2-hydroxy-1-[(3S)-3-methylpiperidin-1-yl]ethyl}-4-(methylsulfanyl)-2-{3-[(1r,3s)-3-methyl-1-(4-methyl-1,2,4-triazol-3-yl)cyclobutyl]phenyl}-3H-isoindol-1-one (23.7 mg, 17%) as a white solid. LCMS:(ES·m/z):546[M+H]⁺.

¹H NMR (400 MHz, DMSO-d₆) δ 8.29-8.25 (m, 2H), 8.05 (s, 1H), 7.71 (d, J=8 Hz, 1H), 7.47 (s, 2H), 7.43 (t, J=8 Hz, 1H), 7.09 (d, J=8 Hz, 1H), 3.85-3.81 (m, 1H), 3.78-3.73 (m, 1H), 3.60-3.57 (m, 1H), 3.26 (s, 3H), 2.95-2.80 (m, 3H), 2.70-2.67 (m, 1H), 2.59-2.54 (m, 6H), 2.08-1.80 (m, 1H), 1.71-1.66 (m, 1H), 1.61-1.58 (m, 2H), 1.51-1.39 (m, 3H), 1.23-1.09 (m, 3H), 0.82-0.74 (m, 4H).

### Synthesis of Compound 171

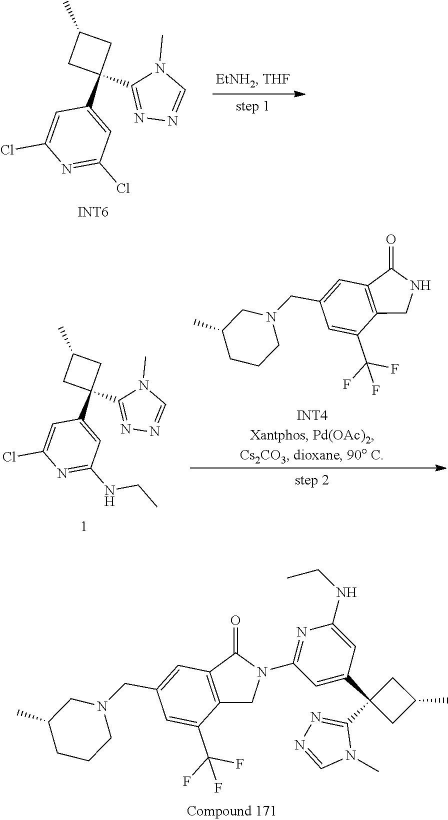

### Step 1: Synthesis of Compound 1

To a stirred solution of 2,6-dichloro-4-[3-methyl-1-(4-methyl-1,2,4-triazol-3-yl)cyclobutyl]pyridine (3 g, 10.09 mmol, 1 equiv) in Ethylamine (2.0 M in THF, 30 mL) was stirred for 2 days at 90° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (12:1) to afford 6-chloro-N-ethyl-4-[(1r,3s)-3-methyl-1-(4-methyl-1,2,4-triazol-3-yl)cyclobutyl]pyridin-2-amine (2.1 g, 66%) as an off-white solid. LCMS (ESI, m/z):306,308[M+H]⁺

### Step 2: Synthesis of Compound 171

To a stirred solution of 6-chloro-N-ethyl-4-[(1r,3s)-3-methyl-1-(4-methyl-1,2,4-triazol-3-yl)cyclobutyl]pyridin-2-amine (40 mg, 0.13 mmol, 1 equiv) and 6-{[(3S)-3-methylpiperidin-1-yl]methyl}-4-(trifluoromethyl)-2,3-dihydroisoindol-1-one (45 mg, 0.14 mmol, 1.1 equiv) in dioxane (1 mL) was added Cs₂CO₃ (85 mg, 0.26 mmol, 2 equiv), Xantphos (15 mg, 0.026 mmol, 0.2 equiv) and Pd(OAc)₂ (3 mg, 0.013 mmol, 0.1 equiv) at room temperature under nitrogen atmosphere. The resulting mixture was stirred for 2 h at 90° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (10:1) to afford 2-[6-(ethylamino)-4-[(1r,3s)-3-methyl-1-(4-methyl-1,2,4-triazol-3-yl)cyclobutyl]pyridin-2-yl]-6-{[(3 S)-3-methylpiperidin-1-yl]methyl}-4-(trifluoromethyl)-3H-isoindol-1-one (31.5 mg, 41%) as a white solid. ¹H NMR (400 MHz, DMSO-d₆) δ 8.34 (s, 1H), 7.93-7.90 (m, 2H), 7.70 (s, 1H), 6.69 (t, J=5.2 Hz, 1H), 6.13 (s, 1H), 5.16 (s, 2H), 3.63 (s, 2H), 3.30-3.25 (m, 2H), 3.27-3.224 (m, 5H), 2.75-2.71 (m, 4H), 2.63-2.61 (m, 3H), 1.95-1.90 (m, 1H), 1.67-1.64 (m, 4H), 1.59-1.49 (m, 1H), 1.17 (t, J=7.2 Hz, 3H), 1.09 (d, J=6.0 Hz, 3H), 0.86-0.81 (m, 4H). LCMS (ESI, m/z):582[M+H]⁺

### Synthesis of Compound 172

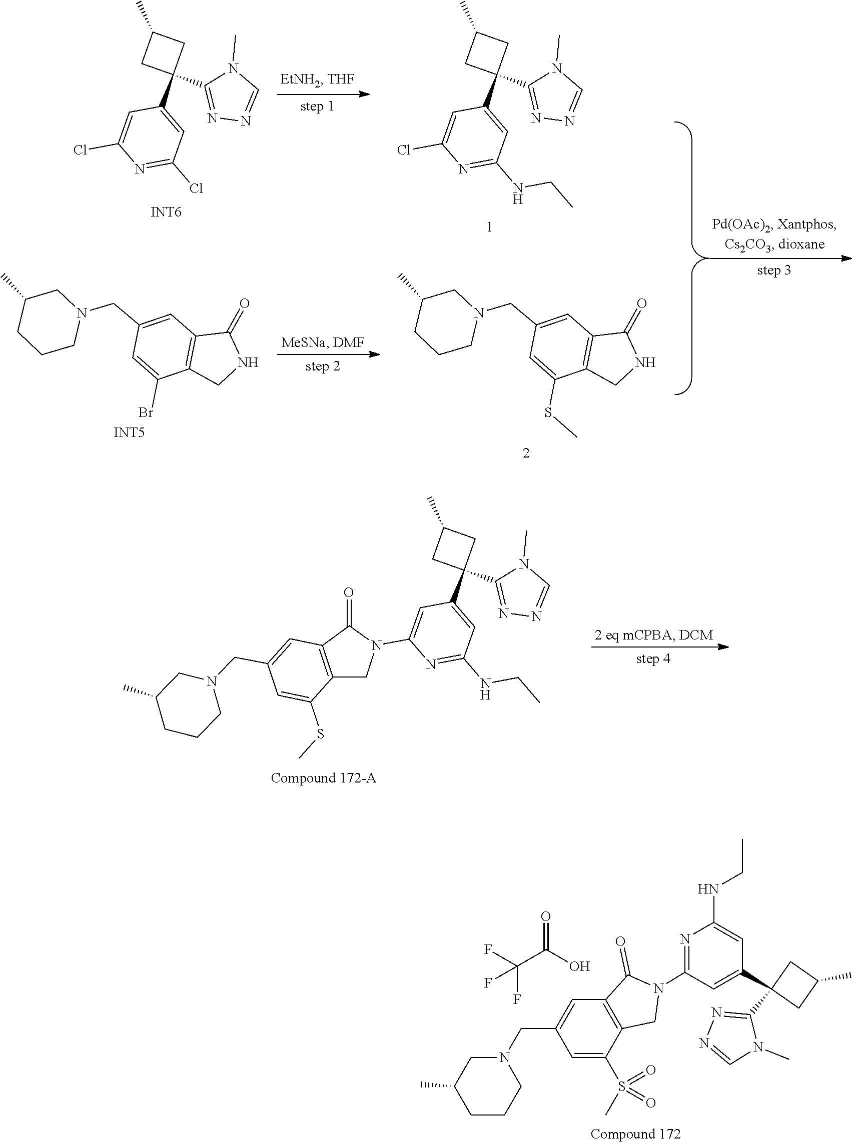

### Step 1: Synthesis of Compound 1

To a stirred solution of 2,6-dichloro-4-[3-methyl-1-(4-methyl-1,2,4-triazol-3-yl)cyclobutyl]pyridine (3 g, 10.09 mmol, 1 equiv) in Ethylamine (2.0 M in THF, 30 mL) was stirred for 2 days at 90° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (12:1) to afford 6-chloro-N-ethyl-4-[(1r,3s)-3-methyl-1-(4-methyl-1,2,4-triazol-3-yl)cyclobutyl]pyridin-2-amine (2.1 g, 66%) as an off-white solid. LCMS (ESI, m/z):306, 308[M+H]⁺

### Step 2: Synthesis of Compound 2

To a stirred solution of 4-bromo-6-{[(3S)-3-methylpiperidin-1-yl]methyl}-2,3-dihydroisoindol-1-one (500 mg, 1.54 mmol, 1 equiv) in DMF (5 mL) was added NaSMe (162 mg, 2.32 mmol, 1.5 equiv) in portions at room temperature under nitrogen atmosphere. The resulting mixture was stirred for 1 h at 90° C. under nitrogen atmosphere. LCMS indicated the reaction was completed. The mixture was allowed to cool down to room temperature. The reaction mixture was purified by reverse flash chromatography with the following conditions: column, C18 silica gel; mobile phase, MeCN in Water (0.05% TFA), 5% to 60% gradient in 30 min; detector, UV 254 nm. The collected fraction was concentrated to afford 6-{[(3S)-3-methylpiperidin-1-yl]methyl}-4-(methylsulfanyl)-2,3-dihydroisoindol-1-one (410 mg, 91%) as a white solid. LCMS (ESI, m/z):291[M+H]⁺

### Step 3: Synthesis of Compound 172-A

To a stirred solution of 6-chloro-N-ethyl-4-[(1r,3s)-3-methyl-1-(4-methyl-1,2,4-triazol-3-yl)cyclobutyl]pyridin-2-amine (100 mg, 0.33 mmol, 1 equiv) and 6-{[(3S)-3-methylpiperidin-1-yl]methyl}-4-(methylsulfanyl)-2,3-dihydroisoindol-1-one (104 mg, 0.36 mmol, 1.1 equiv) in dioxane (2 mL) was added Cs₂CO₃ (213 mg, 0.65 mmol, 2 equiv), Xantphos (38 mg, 0.065 mmol, 0.2 equiv) and Pd(OAc)₂ (7 mg, 0.033 mmol, 0.1 equiv) at room temperature under nitrogen atmosphere. The resulting mixture was stirred for 2 h at 120° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (10:1) to afford 2-[6-(ethylamino)-4-[(1r,3s)-3-methyl-1-(4-methyl-1,2,4-triazol-3-yl)cyclobutyl]pyridin-2-yl]-6-{[(3S)-3-methylpiperidin-1-yl]methyl}-4-(methylsulfanyl)-3H-isoindol-1-one (50.4 mg, 27%) as a white solid. ¹H NMR (400 MHz, DMSO-d₆) δ 8.34 (s, 1H), 7.71 (s, 1H), 7.46 (s, 2H), 6.66 (d, J=4.8 Hz, 1H), 6.09 (s, 1H), 4.87 (s, 2H), 3.55 (br s, 2H), 3.27-3.24 (m, 5H), 2.74-2.72 (m, 4H), 2.67-2.54 (m, 6H), 1.91-1.89 (m, 1H), 1.64-1.35 (m, 5H), 1.16 (t, J=7.2 Hz, 3H), 1.08 (d, J=6.0 Hz, 3H), 0.86-0.82 (m, 4H). LCMS (ESI, m/z):560[M+H]⁺

### Step 4: Synthesis of Compound 172

To a stirred solution of 2-[6-(ethylamino)-4-[(1r,3s)-3-methyl-1-(4-methyl-1,2,4-triazol-3-yl)cyclobutyl]pyridin-2-yl]-6-{[(3S)-3-methylpiperidin-1-yl]methyl}-4-(methylsulfanyl)-3H-isoindol-1-one (35 mg, 0.06 mmol, 1 equiv) in DCM (700 uL) was added mCPBA (21 mg, 0.12 mmol, 2 equiv) in portions at room temperature under air atmosphere. The resulting mixture was stirred for 1 h at room temperature under air atmosphere. LCMS indicated the reaction was completed. The resulting mixture was concentrated under reduced pressure. The crude product (35 mg) was purified by Prep-HPLC with the following conditions (Column: XSelect CSH Fluoro Phenyl, 30*150 mm, 5 m; Mobile Phase A: Water (0.05% TFA), Mobile Phase B: ACN; Flow rate: 60 mL/min; Gradient: 5% B to 34% B in 10 min, 34% B; Wave Length: 254 nm; RT1 (min): 8.5; Number Of Runs: The collected fraction was lyophilized to afford 2-[6-(ethylamino)-4-[(1r,3s)-3-methyl-1-(4-methyl-1,2,4-triazol-3-yl)cyclobutyl]pyridin-2-yl]-4-methanesulfonyl-6-{[(3S)-3-methylpiperidin-1-yl]methyl}-3H-isoindol-1-one; trifluoroacetic acid (21.3 mg, 47%) as a yellow solid. LCMS:(ES·m/z):592[M+H−TFA]⁺. ¹H NMR (400 MHz, DMSO-d₆) δ12.53 (br s, 1H), 8.87 (s, 1H), 8.15 (s, 1H), 8.06 (s, 1H), 7.66 (s, 1H), 6.22 (s, 1H), 5.28 (s, 2H), 5.06-4.99 (m, 2H), 3.54-3.45 (m, 3H), 3.41-3.32 (m, 5H), 2.93 (s, 3H), 2.78-2.77 (m, 2H), 2.67-2.51 (m, 4H), 2.18-2.05 (m, 1H), 1.93-1.75 (m, 3H), 1.23-1.00 (m, 7H), 0.89 (d, J=6.6 Hz, 3H).

### Synthesis of Compound 173

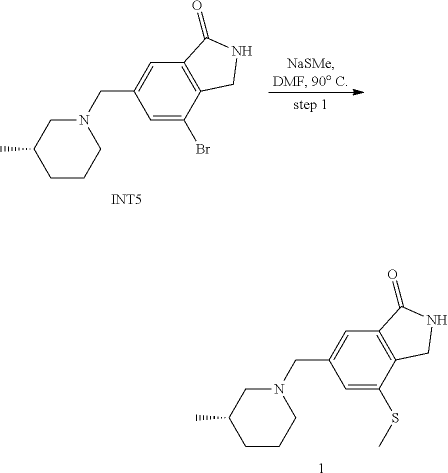

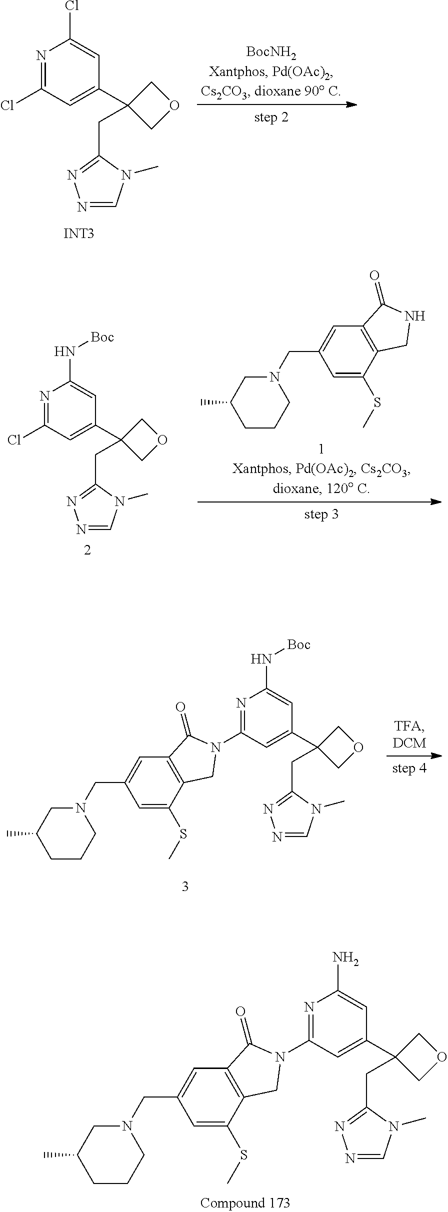

### Step 1: Synthesis of Compound 1

To a stirred solution of 4-bromo-6-([(3S)-3-methylpiperidin-1-yl]methyl-2,3-dihydroisoindol-1-one (2.05 g, 6.34 mmol, 1 equiv) in DMF (20 mL) was added (methylsulfanyl)sodium (0.67 g, 9.51 mmol, 1.5 equiv) at room temperature. The resulting mixture was stirred for 2 h at 90° C. under nitrogen atmosphere. LCMS indicated the reaction was completed. The mixture was allowed to cool down to room temperature. The resulting mixture was concentrated under vacuum. The residue was purified by reversed-phase flash chromatography with the following conditions: column, C18 silica gel; mobile phase, MeCN in Water (10 mmol/L NH₄HCO₃), 5% to 80% gradient in 30 min; detector, UV 254 nm. The collected fraction was concentrated to afford 6-([(3S)-3-methylpiperidin-1-yl]methyl-4-(methylsulfanyl)-2,3-dihydroisoindol-1-one (1.55 g, 84%) as an off-white solid. LCMS (ESI, m/z):291[M+H]⁺

### Step 2: Synthesis of Compound 2

To a stirred solution of 2,6-dichloro-4-(3-[(4-methyl-1,2,4-triazol-3-yl)methyl]oxetan-3-ylpyridine (700 mg, 2.34 mmol, 1 equiv), tert-butyl carbamate (247 mg, 2.10 mmol, 0.9 equiv) and Cs₂CO₃ (1524 mg, 4.68 mmol, 2 equiv) in dioxane (14 mL) was added Xantphos (270 mg, 0.46 mmol, 0.2 equiv) and Pd(OAc)₂ (52 mg, 0.23 mmol, 0.1 equiv) at room temperature. The resulting mixture was stirred for 1 h at 90° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (12:1) to afford tert-butyl N-(6-chloro-4-(3-[(4-methyl-1,2,4-triazol-3-yl)methyl]oxetan-3-ylpyridin-2-yl)carbamate (600 mg, 67%) as an off-white solid. LCMS (ESI, m/z):380,382[M+H]⁺

### Step 3: Synthesis of Compound 3

To a stirred solution of tert-butyl N-(6-chloro-4-(3-[(4-methyl-1,2,4-triazol-3-yl)methyl]oxetan-3-ylpyridin-2-yl)carbamate (580 mg, 1.52 mmol, 1 equiv), 6-([(3S)-3-methylpiperidin-1-yl]methyl-4-(methylsulfanyl)-2,3-dihydroisoindol-1-one (443 mg, 1.52 mmol, 1 equiv) and Cs₂CO₃ (995 mg, 3.05 mmol, 2 equiv) in dioxane (12 mL) was added Xantphos (177 mg, 0.30 mmol, 0.2 equiv) and Pd(OAc)₂ (34 mg, 0.15 mmol, 0.1 equiv) at room temperature. The resulting mixture was stirred for 1 h at 120° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (12:1) to afford tert-butyl N-(4-(3-[(4-methyl-1,2,4-triazol-3-yl)methyl]oxetan-3-yl-6-(6-([(3S)-3-methylpiperidin-1-yl]methyl-4-(methylsulfanyl)-1-oxo-3H-isoindol-2-yl)pyridin-2-yl)carbamate (750 mg, 77%) as an off-white solid. LCMS (ESI, m/z):634[M+H]⁺

### Step 4: Synthesis of Compound 173

To a stirred solution of tert-butyl N-(4-(3-[(4-methyl-1,2,4-triazol-3-yl)methyl]oxetan-3-yl-6-(6-([(3S)-3-methylpiperidin-1-yl]methyl-4-(methylsulfanyl)-1-oxo-3H-isoindol-2-yl)pyridin-2-yl)carbamate (750 mg, 1.18 mmol, 1 equiv) in DCM (15 mL) was added TFA (8 mL) dropwise at 0° C. The resulting mixture was stirred for 2 h at 0° C. under nitrogen atmosphere. LCMS indicated the reaction was completed. The resulting mixture was concentrated under vacuum. The residue was purified by reversed-phase flash chromatography with the following conditions: column, C18 silica gel; mobile phase, MeCN in Water (10 mmol/L NH₄HCO₃), 5% to 80% gradient in 30 min; detector, UV 254 nm. The collected fraction was concentrated under vacuum and lyophilized to afford 2-(6-amino-4-(3-[(4-methyl-1,2,4-triazol-3-yl)methyl]oxetan-3-ylpyridin-2-yl)-6-([(3S)-3-methylpiperidin-1-yl]methyl-4-(methylsulfanyl)-3H-isoindol-1-one (425 mg, 65%) as a white solid. LCMS (ESI, m/z):534[M+H]^{+ 1}H NMR (400 MHz, DMSO-d₆) δ 8.26 (s, 1H), 7.50-7.40 (m, 3H), 6.08-5.91 (m, 3H), 4.90 (d, J=6.0 Hz, 2H), 4.83-4.74 (m, 4H), 3.55-3.48 (m, 2H), 3.23 (s, 3H), 2.72 (t, J=9.2 Hz, 2H), 2.59 (s, 3H), 1.90 (t, J=10.0 Hz, 1H), 1.62 (m, 4H), 1.46-1.43 (m, 1H), 0.86-0.81 (m, 4H).

### Synthesis of Compound 174

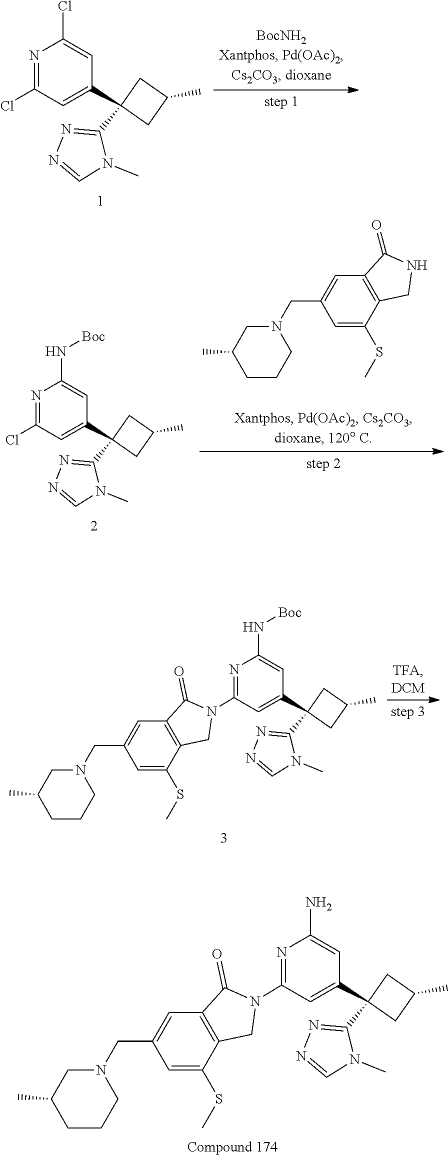

### Step 1: Synthesis of Compound 2

A mixture of 2,6-dichloro-4-[(1r,3s)-3-methyl-1-(4-methyl-1,2,4-triazol-3-yl)cyclobutyl]pyridine (800 mg, 2.69 mmol, 1 equiv), BocNH₂ (315 mg, 2.69 mmol, 1 equiv), XantPhos (311 mg, 0.54 mmol, 0.2 equiv), Pd(OAc)₂ (120 mg, 0.54 mmol, 0.2 equiv) and Cs₂CO₃ (1.75 g, 5.38 mmol, 2 equiv) in dioxane (16 mL) was stirred for 1 h at 90° C. under nitrogen atmosphere. LCMS indicated the reaction was completed. The reaction mixture was allowed to cool down to room temperature and the solvent was removed under reduced pressure. The residue was purified by silica gel column chromatography, eluted with DCM/MeOH (94:6) to afford tert-butyl N-{6-chloro-4-[(1r,3s)-3-methyl-1-(4-methyl-1,2,4-triazol-3-yl)cyclobutyl]pyridin-2-yl}carbamate (510 mg, 50%) as a yellow solid. LCMS (ESI, m/z): 378,380 [M+H]⁺.

### Step 2: Synthesis of Compound 3

A mixture of tert-butyl N-{6-chloro-4-[(1r,3s)-3-methyl-1-(4-methyl-1,2,4-triazol-3-yl)cyclobutyl]pyridin-2-yl}carbamate (710 mg, 1.88 mmol, 1 equiv), 6-{[(3S)-3-methylpiperidin-1-yl]methyl}-4-(methylsulfanyl)-2,3-dihydroisoindol-1-one (545 mg, 1.88 mmol, 1 equiv), Pd(OAc)₂ (84 mg, 0.37 mmol, 0.2 equiv), XantPhos (217 mg, 0.37 mmol, 0.2 equiv) and Cs₂CO₃ (122 mg, 0.37 mmol, 0.2 equiv) in dioxane (15 mL) was stirred for 1 h at 120° C. under nitrogen atmosphere. LCMS indicated the reaction was completed. The reaction mixture was allowed to cool down to room temperature and the solvent was removed under reduced pressure. The residue was purified by silica gel column chromatography, eluted with DCM/MeOH (95:5) to afford tert-butyl N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate (540 mg, 45%) as a yellow solid. LCMS (ESI, m/z): 632 [M+H]⁺.

### Step 3: Synthesis of Compound 174

To a mixture of tert-butyl N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate (440 mg, 0.70 mmol, 1 equiv) in DCM (8 mL) was added TFA (1.6 mL) at 25° C. The resulting mixture was stirred for 1 h at 25° C. LCMS indicated the reaction was completed. The solvent was removed under reduced pressure. The residue was purified by reverse flash chromatography with the following conditions: column, C18 silica gel, 80 g, 20-35 um; mobile phase, water with 0.1% NH₄HCO₃ and ACN (0% to 100% gradient in 50 min); detector, UV 254 nm. The collected fraction was lyophilized to afford 2-{6-amino-4-[(1r,3s)-3-methyl-1-(4-methyl-1,2,4-triazol-3-yl)cyclobutyl]pyridin-2-yl}-6-{[(3S)-3-methylpiperidin-1-yl]methyl}-4-(methylsulfanyl)-3H-isoindol-1-one (300 mg, 81%) as a white solid. LCMS (ESI, m/z): 532 [M+H]⁺. ¹H NMR (400 MHz, DMSO-d₆) δ 8.35 (s, 1H), 7.79 (s, 1H), 7.46-7.45 (m, 2H), 6.14-6.08 (m, 3H), 4.83 (s, 2H), 3.55 (s, 2H), 3.25 (s, 3H), 2.78-2.68 (m, 4H), 2.59 (s, 3H), 2.57-2.55 (m, 3H), 1.95-1.84 (m, 1H), 1.70-1.55 (m, 4H), 1.52-1.43 (m, 1H), 1.09 (d, J=56.0 Hz, 3H), 0.88-0.80 (m, 4H).

### Synthesis of Compound 175

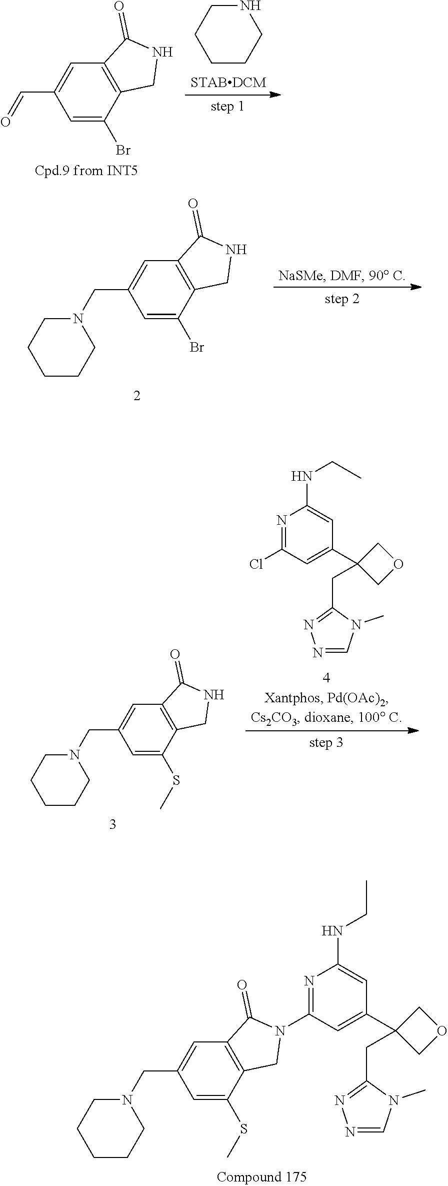

### Step 1: Synthesis of Compound 2

A mixture of 7-bromo-3-oxo-1,2-dihydroisoindole-5-carbaldehyde (600 mg, 2.50 mmol, 1 equiv) and piperidine (212 mg, 2.50 mmol, 1 equiv) in DCM (14 mL) was stirred for 30 min at 25° C. STAB (1.58 g, 7.50 mmol, 3 equiv) was added into the above mixture. The resulting mixture was stirred for 4 h. LCMS indicated the reaction was completed. The reaction was quenched by addition of water (50 mL) and extracted with DCM (3×50 mL). The combined organic layers were dried over anhydrous sodium sulfate. After filtration, the filtrate was concentrated under reduced pressure. The residue was purified by reverse phase flash with the following conditions (column, C18 silica gel, 80 g, 20-35 um; mobile phase, water with 0.1% NH₄HCO₃ and ACN (0% to 100% gradient in 50 min); detector, UV 254 nm) to afford 4-bromo-6-(piperidin-1-ylmethyl)-2,3-dihydroisoindol-1-one (450 mg, 58%) as a white solid. LCMS (ESI, m/z): 309,311 [M+H]⁺.

### Step 2: Synthesis of Compound 3

A mixture of 4-bromo-6-(piperidin-1-ylmethyl)-2,3-dihydroisoindol-1-one (290 mg, 0.94 mmol, 1 equiv) and MeSNa (164 mg, 2.35 mmol, 2.5 equiv) in DMF (4 mL) was stirred for 1 h at 90° C. LCMS indicated the reaction was completed. The reaction mixture was allowed down to room temperature. The mixture was purified by reverse flash chromatography with the following conditions: column, C18 silica gel, 80 g, 20-35 um; mobile phase, water with 0.1% NH₄HCO₃ and ACN (0% to 100% gradient in 50 min); detector, UV 254 nm. The collected fraction was concentrated to afford 4-(methylsulfanyl)-6-(piperidin-1-ylmethyl)-2,3-dihydroisoindol-1-one (120 mg, 46%) as a white solid. LCMS (ESI, m/z): 277 [M+H]⁺.

### Step 3: Synthesis of Compound 175

A mixture of 4-(methylsulfanyl)-6-(piperidin-1-ylmethyl)-2,3-dihydroisoindol-1-one (150 mg, 0.54 mmol, 1 equiv), 6-chloro-N-ethyl-4-{3-[(4-methyl-1,2,4-triazol-3-yl)methyl]oxetan-3-yl}pyridin-2-amine (167 mg, 0.54 mmol, 1 equiv), XantPhos (62 mg, 0.11 mmol, 0.2 equiv), Pd(OAc)₂ (24 mg, 0.11 mmol, 0.2 equiv) and Cs₂CO₃ (353 mg, 1.08 mmol, 2 equiv) in dioxane (4 mL) was stirred for 1 h at 100° C. under nitrogen atmosphere. LCMS indicated the reaction was completed. The reaction mixture was allowed down to room temperature. The reaction was filtered by silica and filtrate was concentrated. The residue was purified by prep-HPLC with following conditions: Column: XBridge Prep OBD C18 Column, 30*150 mm, 5 m; Mobile Phase A: Water (10 mmol/L NH₄HCO₃+0.05% NH₃H₂O), Mobile Phase B: ACN; Flow rate: 60 mL/min mL/min; Gradient: 32% B to 62% B in 8 min; Wave Length: 254 nm/220 nm; RT1 (min): 7.4. The collected fraction was lyophilized to afford 2-[6-(ethylamino)-4-{3-[(4-methyl-1,2,4-triazol-3-yl)methyl]oxetan-3-yl}pyridin-2-yl]-4-(methylsulfanyl)-6-(piperidin-1-ylmethyl)-3H-isoindol-1-one (92.4 mg, 30.71%) as a white solid. LCMS (ES, m/z): 548 [M+H]⁺.

¹H NMR (400 MHz, DMSO-d₆) δ 8.24 (s, 1H), 7.46 (d, J=5.2 Hz, 2H), 7.39 (d, J=0.8 Hz, 1H), 6.56 (t, J=5.2 Hz, 1H), 5.87 (d, J=0.8 Hz, 1H), 4.89 (d, J=6.0 Hz, 2H), 4.84 (s, 2H), 4.78 (d, J=6.0 Hz, 2H), 3.55 (s, 2H), 3.48 (s, 2H), 3.22-3.18 (m, 5H), 2.59 (s, 3H), 2.40-2.30 (m, 4H), 1.56-1.46 (m, 4H), 1.44-1.37 (m, 2H), 1.13 (t, J=7.2 Hz, 3H).

### Synthesis of Compound 176

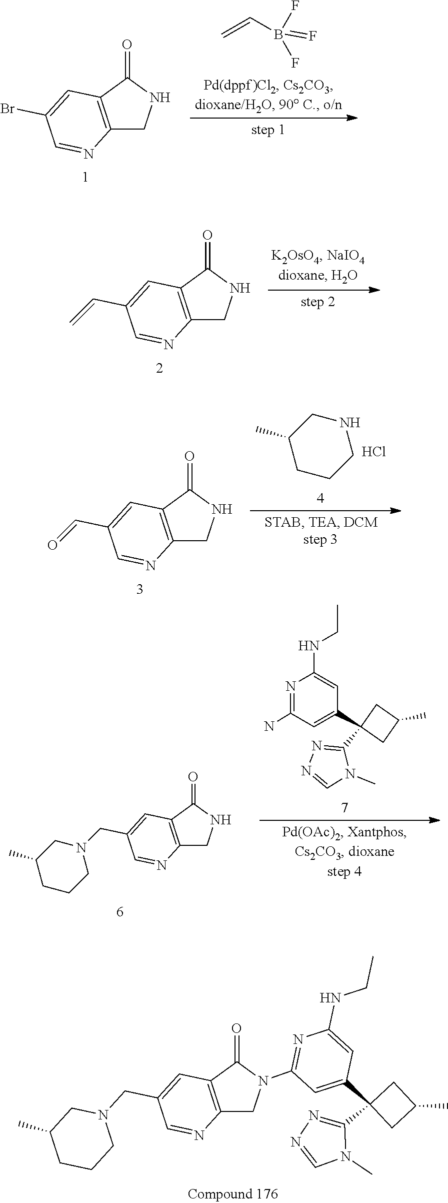

### Step 1: Synthesis of Compound 2

A mixture of 3-bromo-6H,7H-pyrrolo[3,4-b]pyridin-5-one (2 g, 9.39 mmol, 1 equiv), ethenyltrifluoroboranuide (890 mg, 9.39 mmol, 1 equiv), Pd(dppf)Cl₂ (1.37 g, 1.88 mmol, 0.2 equiv) and Cs₂CO₃ (6.11 g, 18.78 mmol, 2 equiv) in dioxane (40 mL) and water (10 mL) was stirred for 1 h at 90° C. under nitrogen atmosphere. LCMS indicated the reaction was completed. The reaction mixture was allowed down to room temperature and the solvent was removed under reduced pressure. The residue was purified by silica gel column chromatography, eluted with DCM/MeOH (95:5) to afford 3-ethenyl-6H,7H-pyrrolo[3,4-b]pyridin-5-one (1.1 g, 73%) as a yellow solid. LCMS (ESI, m/z): 161 [M+H]⁺.

### Step 2: Synthesis of Compound 3

A mixture of 3-ethenyl-6H,7H-pyrrolo[3,4-b]pyridin-5-one (500 mg, 3.12 mmol, 1 equiv), K₂OsO₄·2H₂O (115 mg, 0.31 mmol, 0.1 equiv) and NaIO₄ (1.33 g, 6.244 mmol, 2 equiv) in dioxane (10 mL) and water (2.50 mL) was stirred for 3 h at 25° C. under nitrogen atmosphere. LCMS indicated the reaction was completed. The solvent was removed under reduced pressure. The residue was purified by silica gel column chromatography, eluted with DCM/MeOH (92:8) to afford 5-oxo-6H,7H-pyrrolo[3,4-b]pyridine-3-carbaldehyde (140 mg, 28%) as a yellow solid. LCMS (ESI, m/z): 163 [M+H]⁺.

### Step 3: Synthesis of Compound 6

A mixture of 5-oxo-6H,7H-pyrrolo[3,4-b]pyridine-3-carbaldehyde (140 mg, 0.86 mmol, 1 equiv), TEA (262 mg, 2.59 mmol, 3 equiv) and (3S)-3-methylpiperidine hydrochloride (140 mg, 1.04 mmol, 1.2 equiv) in DCM (3 mL) was stirred for 30 min at 25° C. Then STAB (365 mg, 1.73 mmol, 2 equiv) was added into the above mixture at 25° C. The resulting mixture was stirred for 3 h at 25° C. LCMS indicated the reaction was completed. The reaction was quenched with saturated sodium bicarbonate solution and the organic phase was removed under reduced pressure. The residue was purified by reverse flash chromatography with the following conditions: column, C18 silica gel, 80 g, 20-35 um; mobile phase, water with 0.1% NH₄HCO₃ and ACN (0% to 100% gradient in 30 min); detector, UV 254 nm. The collected fraction was concentrated to afford 3-{[(3S)-3-methylpiperidin-1-yl]methyl}-6H,7H-pyrrolo[3,4-b]pyridin-5-one (70 mg, 33%) as a yellow solid. LCMS (ES, m/z): 246 [M+H]⁺.

### Step 4: Synthesis of Compound 176

A mixture of 3-{[(3S)-3-methylpiperidin-1-yl]methyl}-6H,7H-pyrrolo[3,4-b]pyridin-5-one (60 mg, 0.24 mmol, 1 equiv), 6-chloro-N-ethyl-4-[(1r,3s)-3-methyl-1-(4-methyl-1,2,4-triazol-3-yl)cyclobutyl]pyridin-2-amine (74 mg, 0.24 mmol, 1 equiv), Pd(OAc)₂ (11 mg, 0.049 mmol, 0.2 equiv), XantPhos (28 mg, 0.049 mmol, 0.2 equiv) and Cs₂CO₃ (160 mg, 0.49 mmol, 2 equiv) in dioxane (2.5 mL) was stirred for 1.5 h at 100° C. under nitrogen atmosphere. LCMS indicated the reaction was completed. The reaction mixture was allowed down to room temperature and the solvent was removed under reduced pressure. The residue was dissolved with MeOH (10 mL) and the resulting mixture was filtered. The filtrate was concentrated. The crude product was purified with prep-HPLC with following conditions: Column: XBridge Prep OBD C18 Column, 30*150 mm, 5 m; Mobile Phase A: Water (10 mmol/L NH₄HCO₃+0.05% NH₃H₂O), Mobile Phase B: ACN; Flow rate: 60 mL/min mL/min; Gradient: 33% B to 63% B in 8 min; Wave Length: 254 nm/220 nm; RT1 (min): 6.8. The collection fraction was lyophilized to afford 6-[6-(ethylamino)-4-[(1r,3s)-3-methyl-1-(4-methyl-1,2,4-triazol-3-yl)cyclobutyl]pyridin-2-yl]-3-{[(3S)-3-methylpiperidin-1-yl]methyl}-7H-pyrrolo[3,4-b]pyridin-5-one (23.6 mg, 18.58%) as a yellow solid. LCMS (ESI, m/z): 515 [M+H]⁺. ¹H NMR (400 MHz, DMSO-d₆) δ 8.72 (d, J=1.6 Hz, 1H), 8.34 (s, 1H), 7.99 (d, J=1.6 Hz, 1H), 7.70 (d, J=0.8 Hz, 1H), 6.68 (t, J=5.2 Hz, 1H), 6.12 (d, J=0.8 Hz, 1H), 5.04 (s, 2H), 3.59 (s, 2H), 3.32-3.26 (m, 2H), 3.24 (s, 3H), 2.79-2.65 (m, 4H), 2.63-2.52 (m, 3H), 1.96-1.86 (m, 1H), 1.70-1.55 (m, 4H), 1.53-1.40 (m, 1H) 1.16 (t, J=7.1 Hz, 3H), 1.09 (d, J=5.9 Hz, 3H), 0.88-0.75 (m, 4H).

### Synthesis of Compound 177

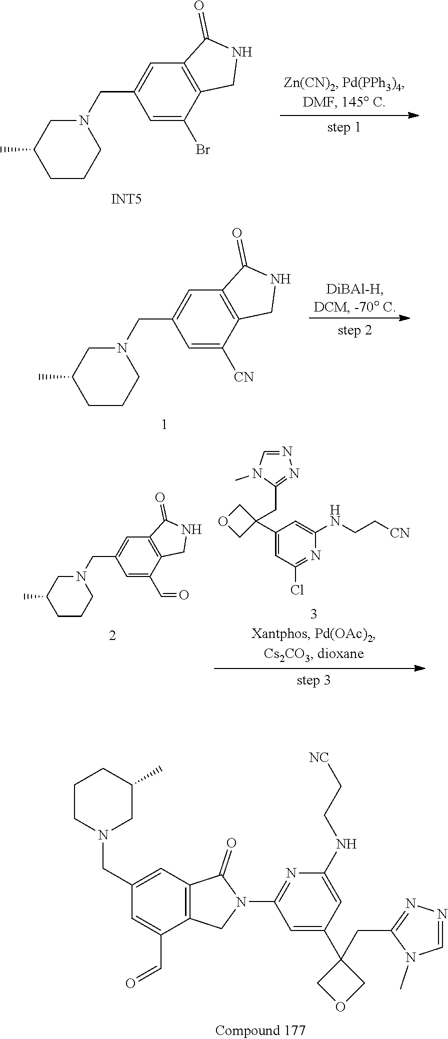

### Step 1: Synthesis of Compound 1

To a stirred solution of 4-bromo-6-{[(3S)-3-methylpiperidin-1-yl]methyl}-2,3-dihydroisoindol-1-one (500 mg, 1.55 mmol, 1 equiv) and Zn(CN)₂ (363 mg, 3.09 mmol, 2 equiv) in DMF (5 mL) was added Pd(PPh₃)₄ (178 mg, 0.15 mmol, 0.1 equiv) at room temperature under nitrogen atmosphere. The resulting mixture was stirred for 2 h at 145° C. under nitrogen atmosphere. LCMS indicated the reaction was completed. The mixture was allowed to cool down to room temperature. The residue was purified by reverse flash chromatography with the following conditions: column, C18 silica gel; mobile phase, MeCN in Water (0.05% TFA), 5% to 60% gradient in 30 min; detector, UV 254 nm. The collected fraction was concentrated to afford 6-{[(3S)-3-methylpiperidin-1-yl]methyl}-1-oxo-2,3-dihydroisoindole-4-carbonitrile (400 mg, 96%) as a brown solid. LCMS (ESI, m/z):270[M+H]⁺

### Step 2: Synthesis of Compound 2

To a stirred solution of 6-{[(3S)-3-methylpiperidin-1-yl]methyl}-1-oxo-2,3-dihydroisoindole-4-carbonitrile (320 mg, 1.19 mmol, 1 equiv) in DCM (10 mL) was added DIBAl-H (3.6 mL, 3.6 mmol, 3.07 equiv, 1 N in DCM) dropwise at −70° C. under nitrogen atmosphere. The resulting mixture was stirred for 2 h at room temperature under nitrogen atmosphere. LCMS indicated the reaction was completed. The reaction was quenched by the addition of MeOH (5 mL) at −20° C. The resulting mixture was concentrated under reduced pressure. The residue was purified by reversed-phase flash chromatography with the following conditions: column, C18 silica gel; mobile phase, MeCN in Water (0.05% TFA), 0% to 50% gradient in 30 min; detector, UV 254 nm. The collected fraction was concentrated to afford 6-{[(3S)-3-methylpiperidin-1-yl]methyl}-1-oxo-2,3-dihydroisoindole-4-carbaldehyde (320 mg, 98%) as a yellow solid. LCMS (ESI, m/z):273[M+H]⁺

### Step 3: Synthesis of Compound 177

To a stirred mixture of 6-{[(3S)-3-methylpiperidin-1-yl]methyl}-1-oxo-2,3-dihydroisoindole-4-carbaldehyde (50 mg, 0.18 mmol, 1 equiv) and 3-[(6-chloro-4-{3-[(4-methyl-1,2,4-triazol-3-yl)methyl]oxetan-3-yl}pyridin-2-yl)amino]propanenitrile (61 mg, 0.18 mmol, 1 equiv) in dioxane (2 mL) was added Cs₂CO₃ (120 mg, 0.37 mmol, 2 equiv), Xantphos (42 mg, 0.036 mmol, 0.2 equiv) and Pd(OAc)₂ (8 mg, 0.018 mmol, 0.1 equiv) at room temperature under air atmosphere. The resulting mixture was stirred for 1.5 h at 110° C. under nitrogen atmosphere. The mixture was allowed to cool down to room temperature. LCMS indicated the reaction was completed. The resulting mixture was concentrated under reduced pressure. The residue was purified by Prep-HPLC with the following conditions (Column: YMC-Actus Triart C18 ExRS, 20*250 mm, 5 m; Mobile Phase A: Water (10 mmol/L NH₄HCO₃), Mobile Phase B: ACN; Flow rate: 25 mL/min; Gradient: 37% B to 67% B in 7 min, 67% B; Wave Length: 254 nm; RT1 (min): 6.05; Injection Volume: 0.6 mL; The collected fraction was lyophilized to afford 3-{[6-(4-formyl-6-{[(3S)-3-methylpiperidin-1-yl]methyl}-1-oxo-3H-isoindol-2-yl)-4-{3-[(4-methyl-1,2,4-triazol-3-yl)methyl]oxetan-3-yl}pyridin-2-yl]amino}propanenitrile (6.5 mg, 6%) as a light yellow solid. LCMS (ESI, m/z):569[M+H]^{+ 1}H NMR (400 MHz, DMSO-d₆) δ10.22 (s, 1H), 8.24 (s, 1H), 8.18 (s, 1H), 7.94 (s, 1H), 7.49 (s, 1H), 7.05 (t, J=6.4 Hz, 1H), 6.05 (s, 1H), 5.30 (s, 2H), 4.91 (d, J=6.0 Hz, 2H), 4.80 (d, J=6.0 Hz, 2H), 3.64 (s, 2H), 3.55-3.52 (m, 4H), 3.24 (s, 3H), 2.84 (t, J=8.0 Hz, 2H), 2.73 (t, J=8.0 Hz, 2H), 1.95 (t, J=8.0 Hz, 1H), 1.64-1.61 (m, 4H), 1.50-1.47 (m, 1H), 0.90-0.80 (m, 4H).

### Synthesis of Compound 178

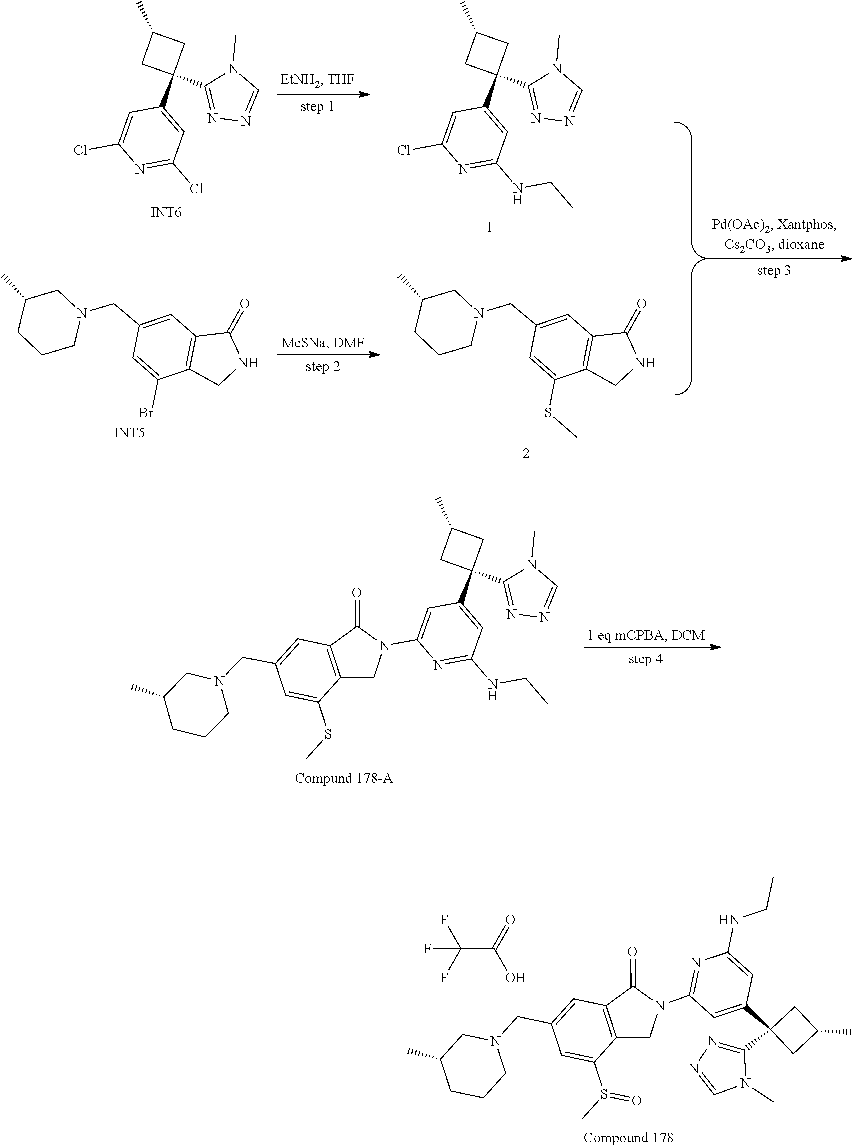

### Step 1: Synthesis of Compound 1

To a stirred solution of 2,6-dichloro-4-[3-methyl-1-(4-methyl-1,2,4-triazol-3-yl)cyclobutyl]pyridine (3 g, 10.09 mmol, 1 equiv) in Ethylamine (2.0 M in THF, 30 mL) was stirred for 2 days at 90° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (12:1) to afford 6-chloro-N-ethyl-4-[(1r,3s)-3-methyl-1-(4-methyl-1,2,4-triazol-3-yl)cyclobutyl]pyridin-2-amine (2.1 g, 66%) as an off-white solid. LCMS (ESI, m/z):306,308[M+H]⁺

### Step 2: Synthesis of Compound 2

To a stirred solution of 4-bromo-6-{[(3S)-3-methylpiperidin-1-yl]methyl}-2,3-dihydroisoindol-1-one (500 mg, 1.54 mmol, 1 equiv) in DMF (5 mL) was added NaSMe (162 mg, 2.32 mmol, 1.5 equiv) in portions at room temperature under nitrogen atmosphere. The resulting mixture was stirred for 1 h at 90° C. under nitrogen atmosphere. LCMS indicated the reaction was completed. The mixture was allowed to cool down to room temperature. The reaction mixture was purified by reverse flash chromatography with the following conditions: column, C18 silica gel; mobile phase, MeCN in Water (0.05% TFA), 5% to 60% gradient in 30 min; detector, UV 254 nm. The collected fraction was concentrated to afford 6-{[(3S)-3-methylpiperidin-1-yl]methyl}-4-(methylsulfanyl)-2,3-dihydroisoindol-1-one (410 mg, 91%) as a white solid. LCMS (ESI, m/z):291[M+H]⁺

### Step 3. Synthesis of Compound 178-A

To a stirred solution of 6-chloro-N-ethyl-4-[(1r,3s)-3-methyl-1-(4-methyl-1,2,4-triazol-3-yl)cyclobutyl]pyridin-2-amine (100 mg, 0.33 mmol, 1 equiv) and 6-{[(3S)-3-methylpiperidin-1-yl]methyl}-4-(methylsulfanyl)-2,3-dihydroisoindol-1-one (104 mg, 0.36 mmol, 1.1 equiv) in dioxane (2 mL) was added Cs₂CO₃ (213 mg, 0.65 mmol, 2 equiv), Xantphos (38 mg, 0.065 mmol, 0.2 equiv) and Pd(OAc)₂ (7 mg, 0.033 mmol, 0.1 equiv) at room temperature under nitrogen atmosphere. The resulting mixture was stirred for 2 h at 120° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (10:1) to afford 2-[6-(ethylamino)-4-[(1r,3s)-3-methyl-1-(4-methyl-1,2,4-triazol-3-yl)cyclobutyl]pyridin-2-yl]-6-{[(3S)-3-methylpiperidin-1-yl]methyl}-4-(methylsulfanyl)-3H-isoindol-1-one (50.4 mg, 27%) as a white solid. ¹H NMR (400 MHz, DMSO-d₆) δ 8.34 (s, 1H), 7.71 (s, 1H), 7.46 (s, 2H), 6.66 (d, J=4.8 Hz, 1H), 6.09 (s, 1H), 4.87 (s, 2H), 3.55 (br s, 2H), 3.27-3.24 (m, 5H), 2.74-2.72 (m, 4H), 2.67-2.54 (m, 6H), 1.91-1.89 (m, 1H), 1.64-1.35 (m, 5H), 1.16 (t, J=7.2 Hz, 3H), 1.08 (d, J=6.0 Hz, 3H), 0.86-0.82 (m, 4H). LCMS (ESI, m/z):560[M+H]⁺

### Step 4: Synthesis of Compound 178

To a stirred solution of 2-[6-(ethylamino)-4-[(1r,3s)-3-methyl-1-(4-methyl-1,2,4-triazol-3-yl)cyclobutyl]pyridin-2-yl]-6-{[(3S)-3-methylpiperidin-1-yl]methyl}-4-(methylsulfanyl)-3H-isoindol-1-one (100 mg, 0.18 mmol, 1 equiv) in DCM (2 mL) was added mCPBA (36 mg, 0.18 mmol, 1 equiv, 85%) in portions at room temperature under air atmosphere. The resulting mixture was stirred for 1 h at room temperature under air atmosphere. LCMS indicated the reaction was 60% product. The resulting mixture was concentrated under reduced pressure. The crude product (100 mg) was purified by Prep-HPLC with the following conditions (Column: XSelect CSH Prep C18 OBD Column, 19*250 mm, 5 m; Mobile Phase A: Water (0.05% TFA), Mobile Phase B: ACN; Flow rate: 25 mL/min; Gradient: 15% B to 45% B in 10 min, 45% B; Wave Length: 220/254 nm; RT1 (min): 9.65; The collected fraction was lyophilized to afford 2-[6-(ethylamino)-4-[(1r,3s)-3-methyl-1-(4-methyl-1,2,4-triazol-3-yl)cyclobutyl]pyridin-2-yl]-4-methanesulfinyl-6-{[(3S)-3-methylpiperidin-1-yl]methyl}-3H-isoindol-1-one (64.2 mg, 52%) as a white solid. LCMS:(ES·m/z):576[M+H−TFA]⁺. ¹H NMR (400 MHz, DMSO-d₆) δ12.30 (br s, 1H), 8.69 (s, 1H), 7.70-7.65 (m, 3H), 6.19 (s, 1H), 4.93 (s, 4H), 3.52-3.44 (m, 3H), 3.31 (s, 3H), 3.28-3.26 (m, 2H), 2.80-2.76 (m, 2H), 2.67-2.59 (m, 4H), 2.56-2.54 (m, 3H), 2.10 (br s, 1H), 1.93-1.87 (m, 1H), 1.82-1.75 (m, 2H), 1.23 (t, J=7.2 Hz, 3H), 1.15-1.08 (m, 4H), 0.90-0.88 (d, J=8 Hz, 3H).

### Synthesis of Compound 179

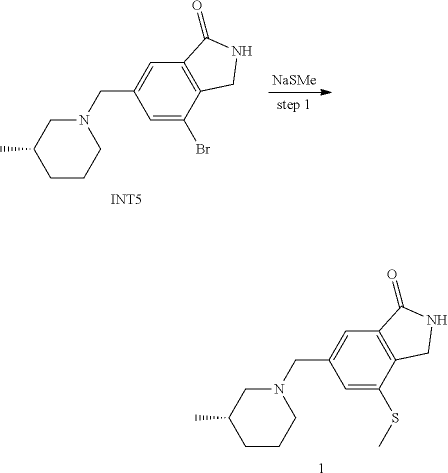

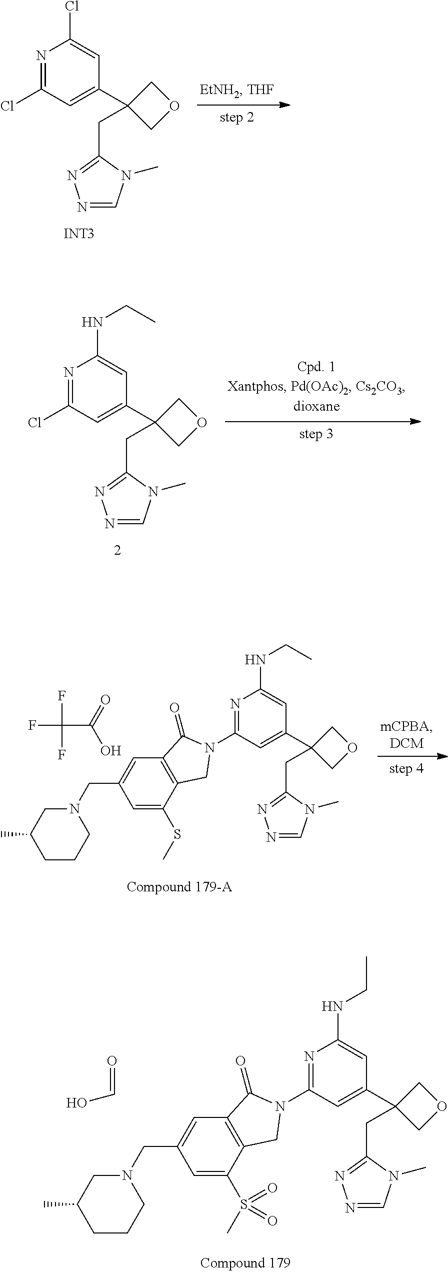

### Step 1: Synthesis of Compound 1

To the solution of 4-bromo-6-{[(3S)-3-methylpiperidin-1-yl]methyl}-2,3-dihydroisoindol-1-one (1.5 g, 4.64 mmol, 1 equiv) in DMF (30 mL) was treated with (methylsulfanyl)sodium (488 mg, 6.96 mmol, 1.5 equiv) at 25° C. The solution was stirred for 1 h at 90° C. LCMS indicated the reaction was completed. The mixture was allowed to cool down to room temperature. The reaction mixture was purified by reverse flash chromatography with the following conditions: column, C18 silica gel, 80 g, 20-35 um; mobile phase, water with 8% NH₄HCO₃ and ACN (0% to 50% gradient in 50 min); detector, UV 254 nm. This resulted in 6-{[(3S)-3-methylpiperidin-1-yl]methyl}-4-(methylsulfanyl)-2,3-dihydroisoindol-1-one (670 mg, 50%) as a white solid. LCMS (ES, m/z): 291 [M+H]⁺

### Step 2; Synthesis of Compound 2

To a stirred solution of 2,6-dichloro-4-{3-[(4-methyl-1,2,4-triazol-3-yl)methyl]oxetan-3-yl}pyridine (800 mg, 2.67 mmol, 1 equiv) in Ethylamine solution (2.0 M in THF, 10 mL) was stirred for overnight at 90° C. under nitrogen atmosphere. LCMS indicated the reaction was completed. The mixture was allowed to cool down to room temperature and concentrated to dryness under vacuum. The residue was purified by silica gel column chromatography, eluted with CH₂Cl₂/MeOH (10:1) to afford 6-chloro-N-ethyl-4-{3-[(4-methyl-1,2,4-triazol-3-yl)methyl]oxetan-3-yl}pyridin-2-amine (450 mg, 54%) as an off-white solid. LCMS (ES, m/z): 308,310 [M+H]⁺

### Step 3. Synthesis of Compound 179-A

To a stirred solution of 6-chloro-N-ethyl-4-{3-[(4-methyl-1,2,4-triazol-3-yl)methyl]oxetan-3-yl}pyridin-2-amine (900 mg, 2.92 mmol, 1 equiv) and 6-{[(3S)-3-methylcyclohexyl]methyl}-4-(methylsulfanyl)-2,3-dihydroisoindol-1-one (846 mg, 2.92 mmol, 1 equiv) in dioxane (18 mL) was added Cs₂CO₃ (1905 mg, 5.84 mmol, 2 equiv), Xantphos (338 mg, 0.58 mmol, 0.2 equiv) and Pd(OAc)₂ (66 mg, 0.29 mmol, 0.1 equiv) at room temperature. The resulting mixture was stirred for 2 h at 120° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (10:1) to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one; trifluoroacetic acid (800 mg) as an off-white solid. The crude product (800 mg) was purified by Prep-HPLC with the following conditions (Column: Xselect CSH C18 OBD Column 30*150 mm 5 m, n; Mobile Phase A: Water (0.05% TFA), Mobile Phase B: ACN; Flow rate: 60 mL/min; Gradient: 10% B to 30% B in 9 min, 30% B; Wave Length: 254; 220 nm; RT1 (min): 8.65; The collected fraction was lyophilized to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one; trifluoroacetic acid (600 mg, 30%) as a yellow solid. LCMS (ES, m/z): 562 [M+H]^{+ 1}H NMR (400 MHz, DMSO-d₆) δ 9.66 (br s, 1H), 8.61 (s, 1H), 7.75-7.68 (m, 2H), 7.37 (s, 1H), 5.96 (s, 1H), 4.90-4.88 (m, 4H), 4.80-4.78 (m, 2H), 4.43-4.42 (m, 2H), 3.57 (s, 2H), 3.38-3.32 (m, 1H), 3.30-3.21 (m, 6H), 2.84-2.81 (m, 1H), 2.64-2.51 (m, 4H), 1.86-1.63 (m, 4H), 1.16-1.04 (m, 4H), 0.94-0.88 (m, 3H)

### Step 4: Synthesis of Compound 179

To a stirred solution of 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (100 mg, 0.18 mmol, 1 equiv) in DCM (2 ml) was added mCPBA (61 mg, 0.36 mmol, 2 equiv) in portions at room temperature under air atmosphere. The resulting mixture was stirred for 1 h at room temperature under air atmosphere. LCMS indicated the reaction was completed. The mixture was concentrated to dryness under vacuum. The crude product was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 32% B to 62% B in 8 min, 62% B; Wave Length: 254; 220 nm; RT1 (min): 7.5; The collected fraction was lyophilized to afford 2-[6-(ethylamino)-4-{3-[(4-methyl-1,2,4-triazol-3-yl)methyl]oxetan-3-yl}pyridin-2-yl]-4-methanesulfonyl-6-{[(3S)-3-methylpiperidin-1-yl]methyl}-3H-isoindol-1-one; formic acid (45.0 mg, 38%) as a yellow solid. LCMS (ES, m/z): 594 [M+H−FA]^{+ 1}H NMR (400 MHz, DMSO-d₆) δ 8.27-8.25 (m, 2H), 8.16 (s, 1H), 8.09 (s, 1H), 7.40 (s, 1H), 6.61 (t, J=5.2 Hz, 1H), 5.88 (s, 1H), 5.23 (s, 2H), 4.90-4.77 (m, 7H), 3.49 (s, 2H), 3.45-3.14 (m, 7H), 3.12-3.03 (m, 1H), 2.90 (s, 3H), 2.22-2.10 (m, 1H), 2.05-1.95 (m, 1H), 1.73-1.66 (m, 2H), 1.13 (t, J=7.2 Hz, 3H), 1.06-0.99 (m, 1H), 0.85 (d, J=6.4 Hz, 3H)

### Synthesis of Compound 180

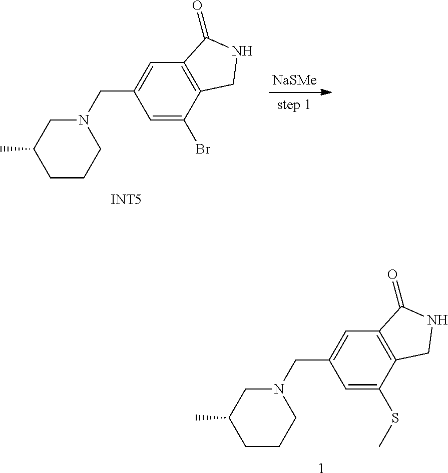

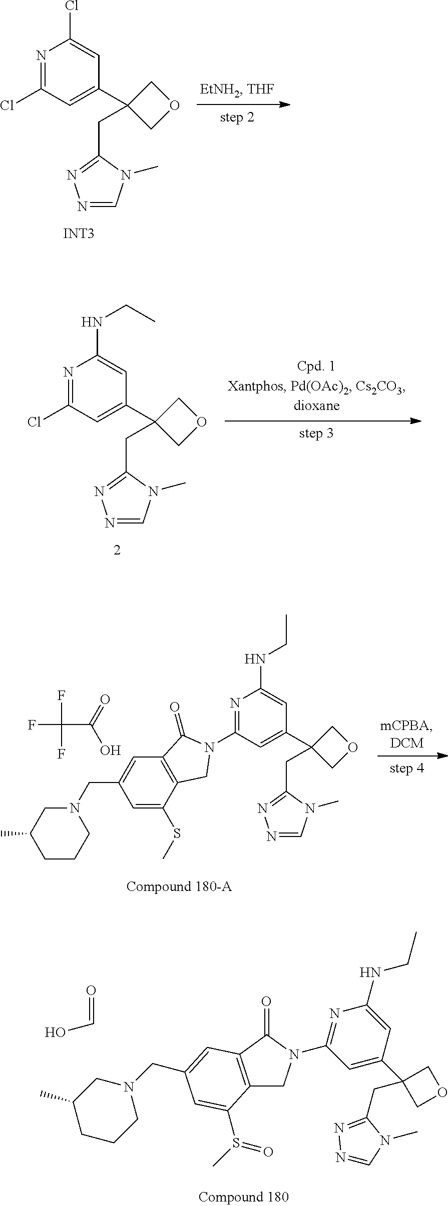

### Step 1: Synthesis of Compound 1

To the solution of 4-bromo-6-{[(3S)-3-methylpiperidin-1-yl]methyl}-2,3-dihydroisoindol-1-one (1.5 g, 4.64 mmol, 1 equiv) in DMF (30 mL) was treated with (methylsulfanyl)sodium (488 mg, 6.96 mmol, 1.5 equiv) at 25° C. The solution was stirred for 1 h at 90° C. LCMS indicated the reaction was completed. The mixture was allowed to cool down to room temperature. The reaction mixture was purified by reverse flash chromatography with the following conditions: column, C18 silica gel, 80 g, 20-35 um; mobile phase, water with 8% NH₄HCO₃ and ACN (0% to 50% gradient in 50 min); detector, UV 254 nm. This resulted in 6-{[(3S)-3-methylpiperidin-1-yl]methyl}-4-(methylsulfanyl)-2,3-dihydroisoindol-1-one (670 mg, 50%) as a white solid. LCMS (ES, m/z): 291 [M+H]⁺

### Step 2: Synthesis of Compound 2

To a stirred solution of 2,6-dichloro-4-{3-[(4-methyl-1,2,4-triazol-3-yl)methyl]oxetan-3-yl}pyridine (800 mg, 2.67 mmol, 1 equiv) in Ethylamine solution (2.0 M in THF, 10 mL) was stirred for overnight at 90° C. under nitrogen atmosphere. LCMS indicated the reaction was completed. The mixture was allowed to cool down to room temperature and concentrated to dryness under vacuum. The residue was purified by silica gel column chromatography, eluted with CH₂Cl₂/MeOH (10:1) to afford 6-chloro-N-ethyl-4-{3-[(4-methyl-1,2,4-triazol-3-yl)methyl]oxetan-3-yl}pyridin-2-amine (450 mg, 54%) as an off-white solid. LCMS (ES, m/z): 308,310 [M+H]⁺

### Step 3: Synthesis of Compound 180-A

To a stirred solution of 6-chloro-N-ethyl-4-{3-[(4-methyl-1,2,4-triazol-3-yl)methyl]oxetan-3-yl}pyridin-2-amine (900 mg, 2.92 mmol, 1 equiv) and 6-{[(3S)-3-methylcyclohexyl]methyl}-4-(methylsulfanyl)-2,3-dihydroisoindol-1-one (846 mg, 2.92 mmol, 1 equiv) in dioxane (18 mL) was added Cs₂CO₃ (1905 mg, 5.84 mmol, 2 equiv), Xantphos (338 mg, 0.58 mmol, 0.2 equiv) and Pd(OAc)₂ (66 mg, 0.29 mmol, 0.1 equiv) at room temperature. The resulting mixture was stirred for 2 h at 120° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (10:1) to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one; trifluoroacetic acid (800 mg) as an off-white solid. The crude product (800 mg) was purified by Prep-HPLC with the following conditions (Column: Xselect CSH C18 OBD Column 30*150 mm 5 m, n; Mobile Phase A: Water (0.05% TFA), Mobile Phase B: ACN; Flow rate: 60 mL/min; Gradient: 10% B to 30% B in 9 min, 30% B; Wave Length: 254; 220 nm; RT1 (min): 8.65; The collected fraction was lyophilized to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one; trifluoroacetic acid (600 mg, 30%) as a yellow solid. LCMS (ES, m/z): 562 [M+H]^{+ 1}H NMR (400 MHz, DMSO-d₆) δ 9.66 (br s, 1H), 8.61 (s, 1H), 7.75-7.68 (m, 2H), 7.37 (s, 1H), 5.96 (s, 1H), 4.90-4.88 (m, 4H), 4.80-4.78 (m, 2H), 4.43-4.42 (m, 2H), 3.57 (s, 2H), 3.38-3.32 (m, 1H), 3.30-3.21 (m, 6H), 2.84-2.81 (m, 1H), 2.64-2.51 (m, 4H), 1.86-1.63 (m, 4H), 1.16-1.04 (m, 4H), 0.94-0.88 (m, 3H)

### Step 4: Synthesis of Compound 180

To a stirred solution of 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (50 mg, 0.089 mmol, 1 equiv) in DCM (2 ml) was added mCPBA (15 mg, 0.089 mmol, 1 equiv) in portions at room temperature under air atmosphere. The resulting mixture was stirred for 1 h at room temperature under air atmosphere. LCMS indicated the reaction was completed. The mixture was concentrated to dryness under vacuum. The crude product was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 32% B to 62% B in 8 min, 62% B; Wave Length: 254; 220 nm; RT1 (min): 7.5; The collected fraction was lyophilized to afford 2-[6-(ethylamino)-4-{3-[(4-methyl-1,2,4-triazol-3-yl)methyl]oxetan-3-yl}pyridin-2-yl]-4-methanesulfinyl-6-{[(3S)-3-methylpiperidin-1-yl]methyl}-3H-isoindol-1-one (18 mg, 33.95%) as a white solid. LCMS (ES, m/z): 578 [M+H−FA]^{+ 1}H NMR (400 MHz, DMSO-d₆) δ 8.31 (br s, 1H), 8.25 (s, 1H), 7.79 (s, 1H), 7.71 (s, 1H), 7.38 (s, 1H), 6.61 (t, J=5.2 Hz, 1H), 5.87 (s, 1H), 4.90-4.86 (m, 4H), 4.78-4.77 (m, 4H), 3.48 (s, 2H), 3.35-3.28 (m, 2H), 3.24-3.17 (m, 6H), 3.07-3.01 (m, 1H), 2.57 (s, 3H), 2.22-2.18 (m, 1H), 2.12-1.95 (m, 1H), 1.72-1.64 (m, 2H), 1.14 (t, J=7.2 Hz, 3H), 1.06-0.98 (m, 1H), 0.85 (d, J=6.4 Hz, 3H).

### Synthesis of Compound 181

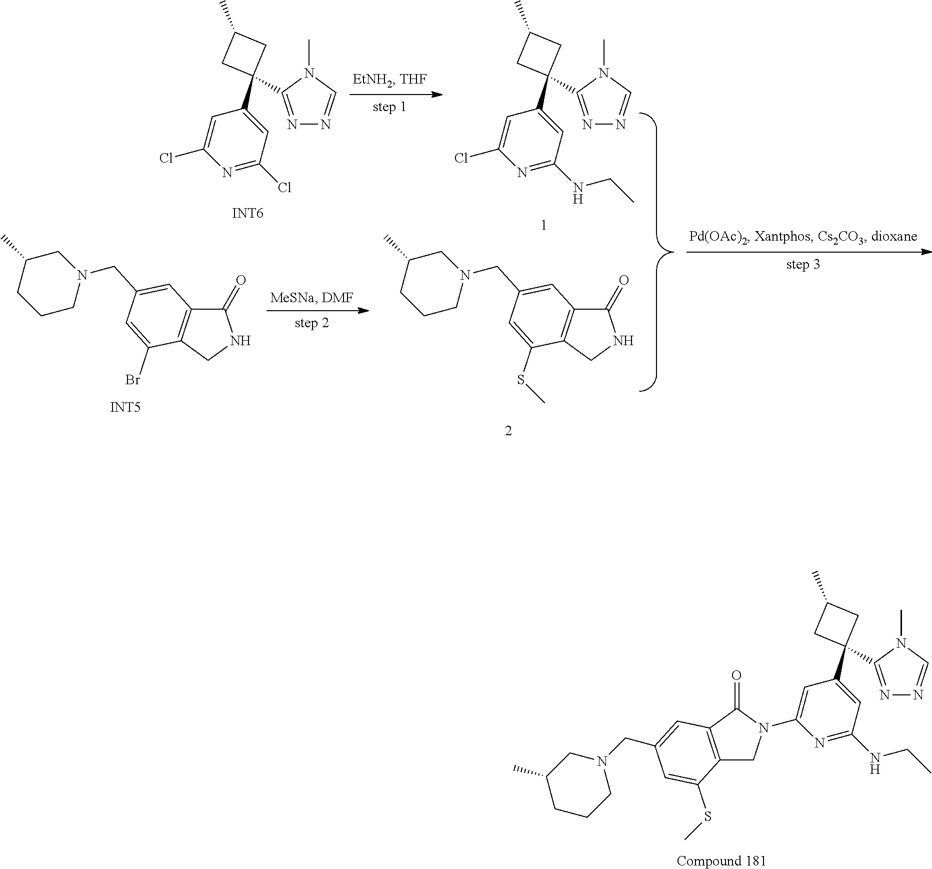

### Step 1: Synthesis of Compound 1

To a stirred solution of 2,6-dichloro-4-[3-methyl-1-(4-methyl-1,2,4-triazol-3-yl)cyclobutyl]pyridine (3 g, 10.09 mmol, 1 equiv) in Ethylamine (2.0 M in THF, 30 mL) was stirred for 2 days at 90° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (12:1) to afford 6-chloro-N-ethyl-4-[(1r,3s)-3-methyl-1-(4-methyl-1,2,4-triazol-3-yl)cyclobutyl]pyridin-2-amine (2.1 g, 66%) as an off-white solid. LCMS (ESI, m/z):306, 308[M+H]⁺

### Step 2: Synthesis of Compound 2

To a stirred solution of 4-bromo-6-{[(3S)-3-methylpiperidin-1-yl]methyl}-2,3-dihydroisoindol-1-one (500 mg, 1.54 mmol, 1 equiv) in DMF (5 mL) was added NaSMe (162 mg, 2.32 mmol, 1.5 equiv) in portions at room temperature under nitrogen atmosphere. The resulting mixture was stirred for 1 h at 90° C. under nitrogen atmosphere. LCMS indicated the reaction was completed. The mixture was allowed to cool down to room temperature. The reaction mixture was purified by reverse flash chromatography with the following conditions: column, C18 silica gel; mobile phase, MeCN in Water (0.05% TFA), 5% to 60% gradient in 30 min; detector, UV 254 nm. The collected fraction was concentrated to afford 6-{[(3S)-3-methylpiperidin-1-yl]methyl}-4-(methylsulfanyl)-2,3-dihydroisoindol-1-one (410 mg, 91%) as a white solid. LCMS (ESI, m/z):291[M+H]⁺

### Step 3: Synthesis of Compound 181

To a stirred solution of 6-chloro-N-ethyl-4-[(1r,3s)-3-methyl-1-(4-methyl-1,2,4-triazol-3-yl)cyclobutyl]pyridin-2-amine (100 mg, 0.33 mmol, 1 equiv) and 6-{[(3S)-3-methylpiperidin-1-yl]methyl}-4-(methylsulfanyl)-2,3-dihydroisoindol-1-one (104 mg, 0.36 mmol, 1.1 equiv) in dioxane (2 mL) was added Cs₂CO₃ (213 mg, 0.65 mmol, 2 equiv), Xantphos (38 mg, 0.065 mmol, 0.2 equiv) and Pd(OAc)₂ (7 mg, 0.033 mmol, 0.1 equiv) at room temperature under nitrogen atmosphere. The resulting mixture was stirred for 2 h at 120° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (10:1) to afford 2-[6-(ethylamino)-4-[(1r,3s)-3-methyl-1-(4-methyl-1,2,4-triazol-3-yl)cyclobutyl]pyridin-2-yl]-6-{[(3S)-3-methylpiperidin-1-yl]methyl}-4-(methylsulfanyl)-3H-isoindol-1-one (50.4 mg, 27%) as a white solid. ¹H NMR (400 MHz, DMSO-d₆) δ 8.34 (s, 1H), 7.71 (s, 1H), 7.46 (s, 2H), 6.66 (d, J=4.8 Hz, 1H), 6.09 (s, 1H), 4.87 (s, 2H), 3.55 (br s, 2H), 3.27-3.24 (m, 5H), 2.74-2.72 (m, 4H), 2.67-2.54 (m, 6H), 1.91-1.89 (m, 1H), 1.64-1.35 (m, 5H), 1.16 (t, J=7.2 Hz, 3H), 1.08 (d, J=6.0 Hz, 3H), 0.86-0.82 (m, 4H). LCMS (ESI, m/z):560[M+H]⁺.

### Synthesis of Compound 182

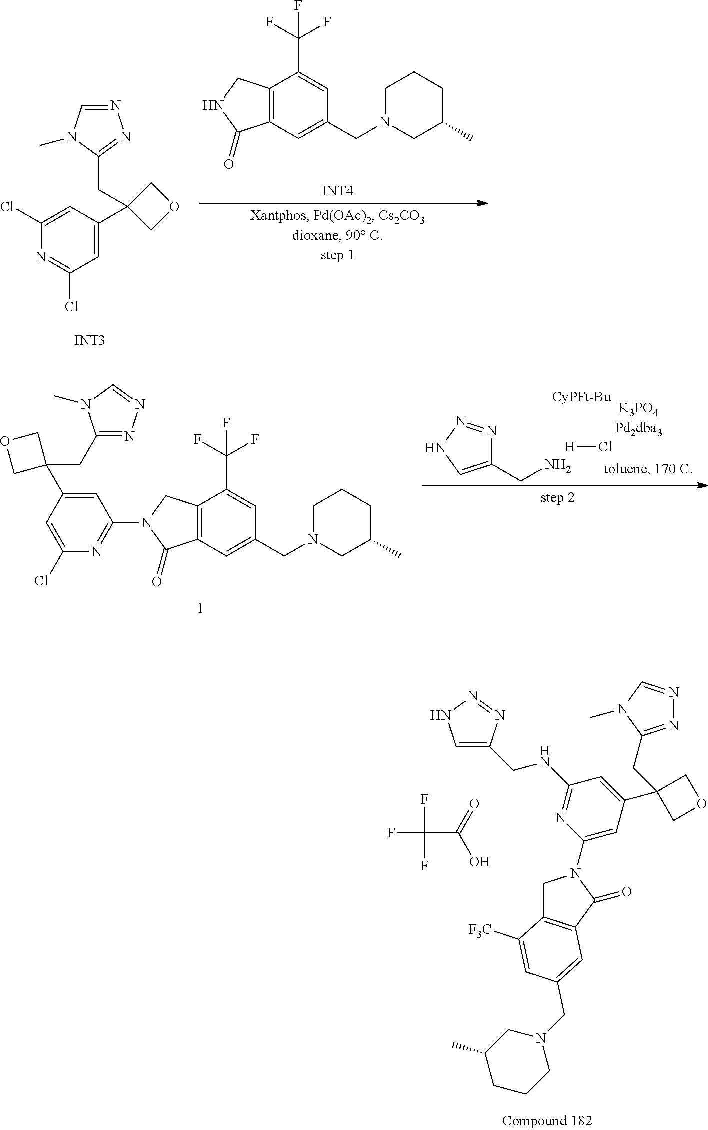

### Step 2: Synthesis of Compound 1

To a stirred mixture of 2,6-dichloro-4-(3-((4-methyl-4H-1,2,4-triazol-3-yl)methyl)oxetan-3-yl)pyridine (235 mg, 0.786 mmol, 1 equiv) and (S)-6-((3-methylpiperidin-1-yl)methyl)-4-(trifluoromethyl)isoindolin-1-one (245 mg, 0.786 mmol, 1 equiv) in dioxane (7 mL) was added Cs₂CO₃ (512 mg, 1.57 mmol, 2 equiv) and Xantphos (90.8 mg, 0.157 mmol, 0.2 equiv) and Pd(OAc)₂ (17.6 mg, 0.0786 mmol, 0.1 equiv) in portions at room temperature. The resulting mixture was stirred for 1 h at 90° C. under nitrogen atmosphere. The mixture was allowed to cool down to room temperature. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (9:1) to afford (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (300 mg, 66.4%) as a green solid. LCMS:(ES·m/z):575,577[M+1]⁺

### Step 2: Synthesis of Compound 182

To a stirred mixture of (S)-2-(6-chloro-4-(3-((4-methyl-4H-1,2,4-triazol-3-yl)methyl)oxetan-3-yl)pyridin-2-yl)-6-((3-methylpiperidin-1l-yl)methyl)-4-(trifluoromethyl)isoindolin-1-one (50 mg, 0.087 mmol, 1 equiv) and (1H-1,2,3-triazol-4-yl)methanamine hydrochloride (35 mg, 0.26 mmol, 3 equiv) in toleune (2 mL) was added potassium phosphate (55 mg, 0.26 mmol, 3 equiv), (R)-1-[(SP)-2-(dicyclohexylphosphino)ferrocenyl]ethyl di-tert-butylphosphine (24 mg, 0.044 mmol, 0.5 equiv) and Pd₂(dba)₃ (16 mg, 0.017 mmol, 0.2 equiv) in portions at room temperature. The resulting mixture was stirred for 25 min at 170° C. under nitrogen atmosphere. The reaction was allowed to cool down to room temperature. LCMS indicated the reaction was completed. The mixture was filtered and purified by reversed-phase flash chromatography with the following conditions: column, C18 silica gel; mobile phase, acetonitrile in water (0.1% TFA), 5% to 50% gradient in 30 min; detector, UV 254 nm. The collected fraction was lyophilized to afford (S)-2-(6-(((1H-1,2,3-triazol-4-yl)methyl)amino)-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one; trifluoroacetic acid (20 mg, 36%) as a yellow solid. LCMS (ESI, m/z):637 [M+H−TFA]⁺, 319 [M/2+H−TFA]^{+ 1}H NMR (500 MHz, MeOD) δ 8.46-8.40 (m, 2H), 8.30 (s, 1H), 8.21 (s, 1H), 8.14 (s, 1H), 7.85 (d, J=1.3 Hz, 1H), 5.44 (s, 2H), 5.16-5.08 (m, 4H), 4.54 (s, 2H), 4.43 (s, 2H), 3.83 (s, 2H), 3.52 (d, J=1.1 Hz, 3H), 3.49 (s, 1H), 3.42 (d, J=12.1 Hz, 1H), 2.96 (t, J=12.2 Hz, 1H), 2.70 (t, J=12.1 Hz, 1H), 1.96 (s, 1H), 1.89 (d, J=12.8 Hz, 2H), 1.77 (d, J=14.2 Hz, 1H), 1.21 (q, J=13.0 Hz, 1H), 1.00 (d, J=6.4 Hz, 3H).

### Synthesis of Compound 183

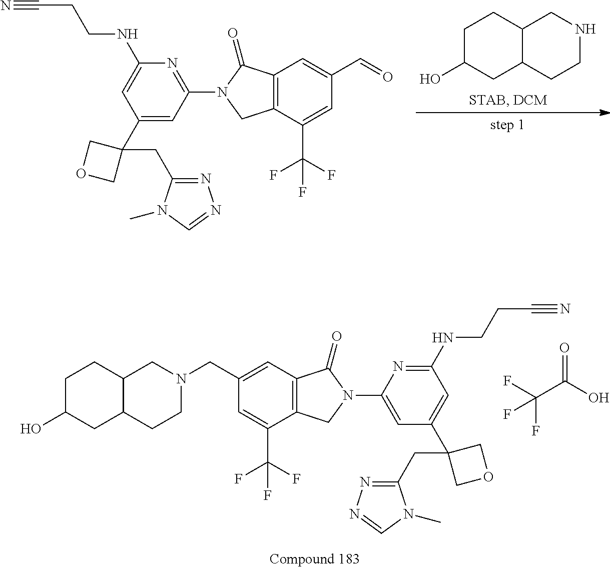

To a stirred solution of 3-((6-(6-formyl-1-oxo-4-(trifluoromethyl)isoindolin-2-yl)-4-(3-((4-methyl-4H-1,2,4-triazol-3-yl)methyl)oxetan-3-yl)pyridin-2-yl)amino)propanenitrile (40 mg, 0.076 mmol, 1 equiv) and decahydroisoquinolin-6-ol (35 mg, 0.23 mmol, 3 equiv) in DCM (3 mL) was added TEA (21 uL, 0.15 mmol, 2 equiv) dropwise at room temperature under nitrogen atmosphere. The resulting mixture was stirred for 30 min at room temperature under nitrogen atmosphere. To the above mixture was added STAB (48 mg, 0.23 mmol, 3 equiv) in portions over at room temperature. The resulting mixture was stirred overnight at room temperature. LCMS indicated the reaction was completed. The reaction was quenched with MeOH at ° C. The resulting mixture was concentrated under vacuum. The residue was purified by reverse flash chromatography with the following conditions: column, C18 silica gel; mobile phase, ACN in Water (0.1% TFA), 5% to 50% gradient in 30 min; detector, UV 280 nm, 254 nm. The eluted was lyophilized to afford 3-((6-(6-((6-hydroxyoctahydroisoquinolin-2 (1H)-yl)methyl)-1-oxo-4-(trifluoromethyl)isoindolin-2-yl)-4-(3-((4-methyl-4H-1,2,4-triazol-3-yl)methyl)oxetan-3-yl)pyridin-2-yl)amino)propanenitrile; trifluoroacetic acid (48 mg, 95%) as a white solid. LCMS (ESI, m/z):665 [M+H−TFA]⁺, 333 [M/2+H−TFA]^{+ 1}H NMR (500 MHz, MeOD) δ 8.69 (s, 1H), 8.23 (d, J=4.4 Hz, 1H), 8.16 (s, 1H), 7.38 (d, J=1.4 Hz, 1H), 6.00 (d, J=1.2 Hz, 1H), 5.33 (s, 2H), 5.04-4.97 (m, 4H), 4.54 (d, J=13.7 Hz, 2H), 3.73 (s, 2H), 3.68 (t, J=6.3 Hz, 2H), 3.62 (dd, J=13.7, 8.5 Hz, 1H), 3.32 (s, 2H), 3.19 (d, J=13.0 Hz, 1H), 2.81 (t, J=6.3 Hz, 2H), 2.23-1.86 (m, 4H), 1.84-1.21 (m, 8H).

### Synthesis of Compound 184

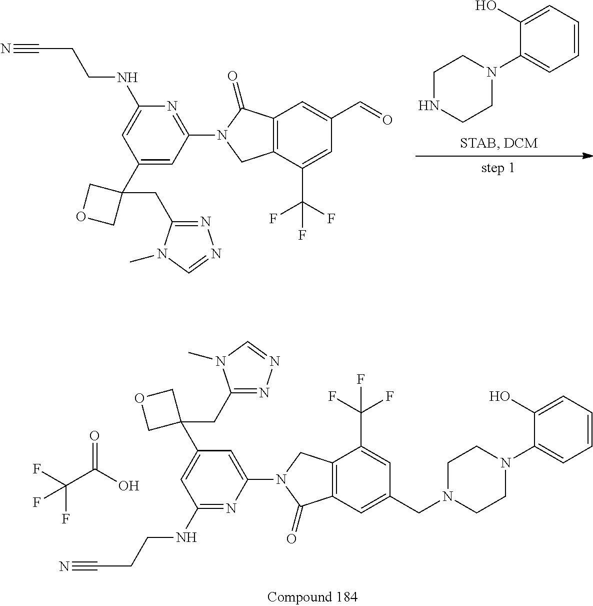 [SMILES: Cn1cnnc1CC1(c2cc(NCCC#N)nc(N3Cc4c(cc(C=O)cc4C(F)(F)F)C3=O)c2)COC1 ; Oc1ccccc1N1CCNCC1 ; O=C(O)C(F)(F)F ; Cn1cnnc1CC1(c2cc(NCCC#N)nc(N3Cc4c(cc(CN5CCN(c6ccccc6O)CC5)cc4C(F)(F)F)C3=O)c2)COC1]

To a stirred solution of 3-((6-(6-formyl-1-oxo-4-(trifluoromethyl)isoindolin-2-yl)-4-(3-((4-methyl-4H-1,2,4-triazol-3-yl)methyl)oxetan-3-yl)pyridin-2-yl)amino)propanenitrile (100 mg, 0.19 mmol, 1 equiv) and 2-(piperazin-1-yl)phenol (102 mg, 0.57 mmol, 3 equiv) in DCM (3 mL) was added TEA (53 uL, 0.38 mmol, 2 equiv) dropwise at room temperature under nitrogen atmosphere. The resulting mixture was stirred for 30 min at room temperature under nitrogen atmosphere. To the above mixture was added STAB (161 mg, 0.76 mmol, 4 equiv) in portions over at room temperature. The resulting mixture was stirred overnight at room temperature. LCMS indicated the reaction was completed. The reaction was quenched with MeOH at ° C. The resulting mixture was concentrated under vacuum. The residue was purified by reverse flash chromatography with the following conditions: column, C18 silica gel; mobile phase, ACN in Water (0.1% TFA), 5% to 50% gradient in 30 min; detector, UV 280 nm, 254 nm. The eluted was lyophilized to afford 3-((6-(6-((4-(2-hydroxyphenyl)piperazin-1-yl)methyl)-1-oxo-4-(trifluoromethyl)isoindolin-2-yl)-4-(3-((4-methyl-4H-1,2,4-triazol-3-yl)methyl)oxetan-3-yl)pyridin-2-yl)amino)propanenitrile; trifluoroacetic acid (25 mg, 19%) as a white solid. LCMS (ESI, m/z):689 [M+H−TFA]⁺, 345 [M/2+H−TFA]⁺; ¹H NMR (500 MHz, MeOD) δ 8.64 (s, 1H), 8.28 (s, 1H), 8.20 (s, 1H), 7.39 (d, J=1.2 Hz, 1H), 7.05-6.95 (m, 2H), 6.88-6.80 (m, 2H), 5.99 (d, J=1.3 Hz, 1H), 5.35 (s, 2H), 5.04-4.97 (m, 4H), 4.62 (s, 2H), 3.74-3.65 (m, 4H), 3.49 (s, 7H), 2.81 (t, J=6.3 Hz, 2H).

### Synthesis of Compound 185

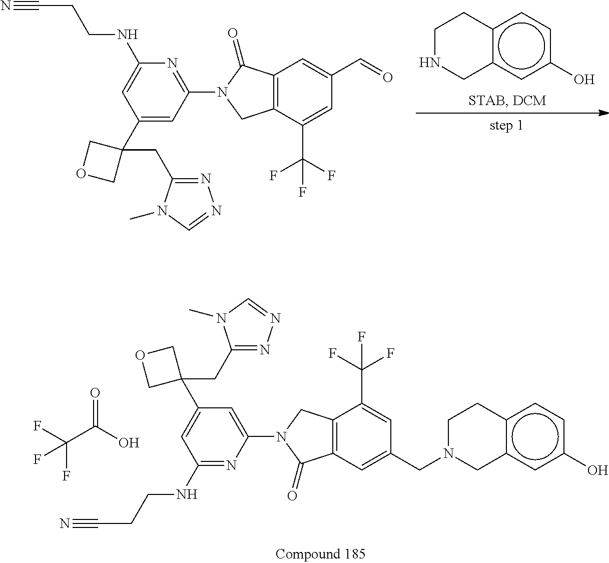

To a stirred solution of 3-((6-(6-formyl-1-oxo-4-(trifluoromethyl)isoindolin-2-yl)-4-(3-((4-methyl-4H-1,2,4-triazol-3-yl)methyl)oxetan-3-yl)pyridin-2-yl)amino)propanenitrile (40 mg, 0.076 mmol, 1 equiv) and 1,2,3,4-tetrahydroisoquinolin-7-ol (11 mg, 0.076 mmol, 1 equiv) in DCM (3 mL) was added TEA (21 uL, 0.15 mmol, 2 equiv) dropwise at room temperature under nitrogen atmosphere. The resulting mixture was stirred for 30 min at room temperature under nitrogen atmosphere. To the above mixture was added STAB (65 mg, 0.3 mmol, 4 equiv) in portions over at room temperature. The resulting mixture was stirred overnight at room temperature. LCMS indicated the reaction was completed. The reaction was quenched with MeOH at ° C. The resulting mixture was concentrated under vacuum. The residue was purified by reverse flash chromatography with the following conditions: column, C18 silica gel; mobile phase, ACN in Water (0.1% TFA), 5% to 50% gradient in 30 min; detector, UV 280 nm, 254 nm. The eluted was lyophilized to afford 3-((6-(6-((7-hydroxy-3,4-dihydroisoquinolin-2 (1H)-yl)methyl)-1-oxo-4-(trifluoromethyl)isoindolin-2-yl)-4-(3-((4-methyl-4H-1,2,4-triazol-3-yl)methyl)oxetan-3-yl)pyridin-2-yl)amino)propanenitrile; trifluoroacetic acid (19 mg, 38%) as a white solid. LCMS (ESI, m/z):659 [M+H−TFA]⁺, 330 [M/2+H−TFA]⁺; ¹H NMR (400 MHz, MeOD) δ 8.72 (s, 1H), 8.29 (s, 1H), 8.21 (s, 1H), 7.39 (d, J=1.3 Hz, 1H), 7.09 (d, J=8.4 Hz, 1H), 6.77 (dd, J=8.4, 2.6 Hz, 1H), 6.57 (d, J=2.5 Hz, 1H), 6.01 (d, J=1.3 Hz, 1H), 5.36 (s, 2H), 5.05-4.90 (m, 4H), 4.84-4.76 (m, 2H), 4.71 (s, 2H), 4.37 (s, 2H), 3.74 (s, 2H), 3.69 (t, J=6.3 Hz, 2H), 3.32 (d, J=10.9 Hz, 6H), 3.11 (d, J=6.7 Hz, 2H), 2.81 (t, J=6.3 Hz, 2H), 1.18 (t, J=7.1 Hz, 1H).

### Synthesis of Compound 186

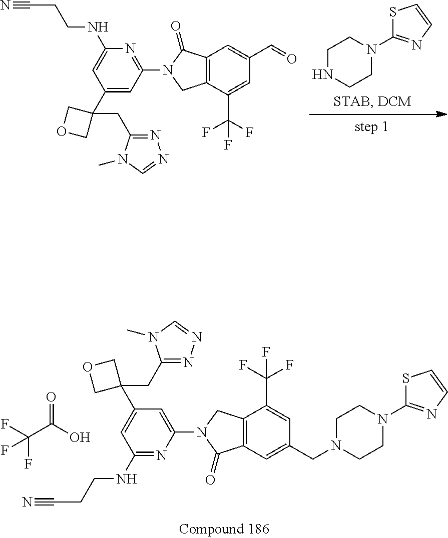

To a stirred solution of 3-((6-(6-formyl-1-oxo-4-(trifluoromethyl)isoindolin-2-yl)-4-(3-((4-methyl-4H-1,2,4-triazol-3-yl)methyl)oxetan-3-yl)pyridin-2-yl)amino)propanenitrile (40 mg, 0.076 mmol, 1 equiv) and 2-(piperazin-1-yl)thiazole (52 mg, 0.3 mmol, 4 equiv) in DCM (3 mL) was added TEA (21 uL, 0.15 mmol, 2 equiv) dropwise at room temperature under nitrogen atmosphere. The resulting mixture was stirred for 30 min at room temperature under nitrogen atmosphere. To the above mixture was added STAB (65 mg, 0.3 mmol, 4 equiv) in portions over at room temperature. The resulting mixture was stirred overnight at room temperature. LCMS indicated the reaction was completed. The reaction was quenched with MeOH at ° C. The resulting mixture was concentrated under vacuum. The residue was purified by reverse flash chromatography with the following conditions: column, C18 silica gel; mobile phase, ACN in Water (0.1% TFA), 5% to 50% gradient in 30 min; detector, UV 280 nm, 254 nm. The eluted was lyophilized to afford 3-((4-(3-((4-methyl-4H-1,2,4-triazol-3-yl)methyl)oxetan-3-yl)-6-(1-oxo-6-((4-(thiazol-2-yl)piperazin-1-yl)methyl)-4-(trifluoromethyl)isoindolin-2-yl)pyridin-2-yl)amino)propanenitrile; trifluoroacetic acid (30 mg, 58%) as a yellow solid. LCMS (ESI, m/z):679 [M+H−TFA]⁺, 340 [M/2+H−TFA]⁺; ¹H NMR (500 MHz, MeOD) δ 8.82 (s, 1H), 8.23 (d, J=1.5 Hz, 1H), 8.15 (s, 1H), 7.36 (d, J=1.3 Hz, 1H), 7.25 (d, J=3.7 Hz, 1H), 6.93 (d, J=3.8 Hz, 1H), 6.03 (d, J=1.3 Hz, 1H), 5.33 (d, J=1.6 Hz, 2H), 5.01 (s, 4H), 4.49 (s, 2H), 3.76 (d, J=10.8 Hz, 6H), 3.69 (t, J=6.4 Hz, 2H), 3.36 (d, J=4.3 Hz, 8H), 2.81 (t, J=6.3 Hz, 2H).

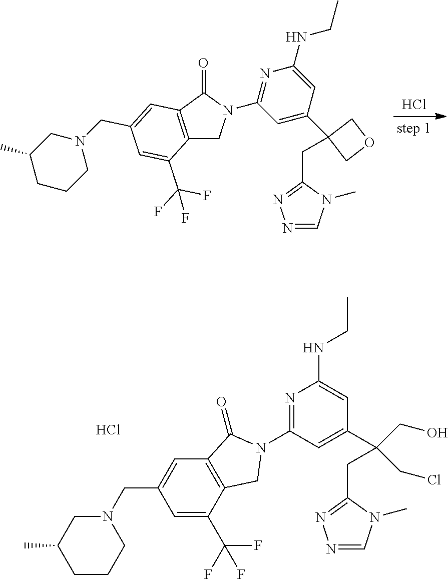 [SMILES: CCNc1cc(C2(Cc3nncn3C)COC2)cc(N2Cc3c(cc(CN4CCC[C@H](C)C4)cc3C(F)(F)F)C2=O)n1 ; Cl ; CCNc1cc(C(CO)(CCl)Cc2nncn2C)cc(N2Cc3c(cc(CN4CCC[C@H](C)C4)cc3C(F)(F)F)C2=O)n1 ; Cl]

To a stirred mixture of (S)-2-(6-(ethylamino)-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (2.2 mg, 0.0038 mmol) in 2 ml of the mixture acetonitrile/water (7:3) was added dropwise HCl (150 uL, 3%). After stirring for 1 hour additional HCl was added dropwise (250 uL, conc.). The mixture was left stirring for two hours at room temperature, then reaction was lyophilized and afforded 2-(4-(1-chloro-3-hydroxy-2-((4-methyl-4H-1,2,4-triazol-3-yl)methyl)propan-2-yl)-6-(ethylamino)pyridin-2-yl)-6-(((S)-3-methylpiperidin-1-yl)methyl)-4-(trifluoromethyl)isoindolin-1-one; hydrochloric acid (2 mg, 85%) as a white solid. LCMS (ESI, m/z): 620 [M+H−HCl]⁺, 310 [M/2+H−HCl]⁺

### Synthesis of Compound 188

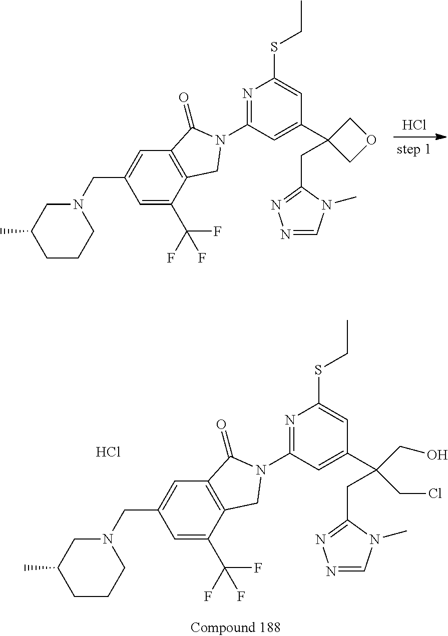 [SMILES: CCSc1cc(C2(Cc3nncn3C)COC2)cc(N2Cc3c(cc(CN4CCC[C@H](C)C4)cc3C(F)(F)F)C2=O)n1 ; Cl ; CCSc1cc(C(CO)(CCl)Cc2nncn2C)cc(N2Cc3c(cc(CN4CCC[C@H](C)C4)cc3C(F)(F)F)C2=O)n1 ; Cl]

To a stirred mixture of (S)-2-(6-(ethylthio)-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (2 mg, 0.003 mmol) in 2 ml of the mixture acetonitrile/water (7:3) was added dropwise HCl (150 uL, 3%). After stirring for 1 hour additional HCl was added dropwise (250 uL, conc.). The mixture was left stirring for two hours at room temperature, then reaction was lyophilized and afforded 2-(4-(1-chloro-3-hydroxy-2-((4-methyl-4H-1,2,4-triazol-3-yl)methyl)propan-2-yl)-6-(ethylthio)pyridin-2-yl)-6-(((S)-3-methylpiperidin-1-yl)methyl)-4-(trifluoromethyl)isoindolin-1-one; hydrochloric acid (2.1 mg, 99%) as a white solid. LCMS (ESI, m/z): 637 [M+H−HCl]⁺, 319 [M/2+H−HCl]⁺

### Synthesis of Compound 189

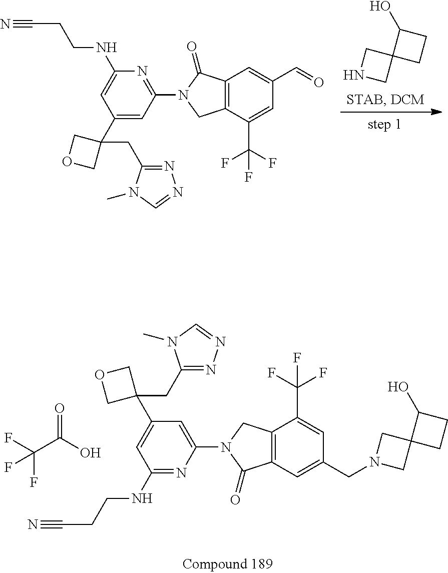

To a stirred solution of 3-((6-(6-formyl-1-oxo-4-(trifluoromethyl)isoindolin-2-yl)-4-(3-((4-methyl-4H-1,2,4-triazol-3-yl)methyl)oxetan-3-yl)pyridin-2-yl)amino)propanenitrile (40 mg, 0.076 mmol, 1 equiv) and 2-azaspiro[3.3]heptan-5-ol (17 mg, 0.15 mmol, 2 equiv) in DCM (3 mL) was added TEA (21 uL, 0.15 mmol, 2 equiv) dropwise at room temperature under nitrogen atmosphere. The resulting mixture was stirred for 30 min at room temperature under nitrogen atmosphere. To the above mixture was added STAB (65 mg, 0.3 mmol, 4 equiv) in portions over at room temperature. The resulting mixture was stirred overnight at room temperature. LCMS indicated the reaction was completed. The reaction was quenched with MeOH at ° C. The resulting mixture was concentrated under vacuum. The residue was purified by reverse flash chromatography with the following conditions: column, C18 silica gel; mobile phase, ACN in Water (0.1% TFA), 5% to 50% gradient in 30 min; detector, UV 280 nm, 254 nm. The eluted was lyophilized to afford 3-((6-(6-((5-hydroxy-2-azaspiro[3.3]heptan-2-yl)methyl)-1-oxo-4-(trifluoromethyl)isoindolin-2-yl)-4-(3-((4-methyl-4H-1,2,4-triazol-3-yl)methyl)oxetan-3-yl)pyridin-2-yl)amino)propanenitrile; trifluoroacetic acid (37 mg, 78%) as a yellow solid. LCMS (ESI, m/z):623 [M+H−TFA]⁺, 312 [M/2+H−TFA]⁺; ¹H NMR (500 MHz, MeOD) δ 8.88-8.79 (m, 1H), 8.19 (s, 1H), 8.10 (s, 1H), 7.36 (dt, J=2.6, 1.2 Hz, 1H), 6.02 (d, J=4.0 Hz, 1H), 5.32 (s, 2H), 5.00 (s, 4H), 4.72 (s, 1H), 4.59 (d, J=3.4 Hz, 3H), 4.44 (s, OH), 4.15 (dt, J=29.6, 9.3 Hz, 4H), 3.75 (d, J=3.8 Hz, 2H), 3.68 (t, J=6.3 Hz, 2H), 3.38-3.33 (m, 4H), 2.81 (t, J=6.3 Hz, 2H), 2.17 (d, J=9.8 Hz, 1H), 2.06-1.97 (m, 1H), 1.83 (q, J=10.5 Hz, 1H), 1.67 (p, J=10.2 Hz, 1H).

### Synthesis of Compound 190

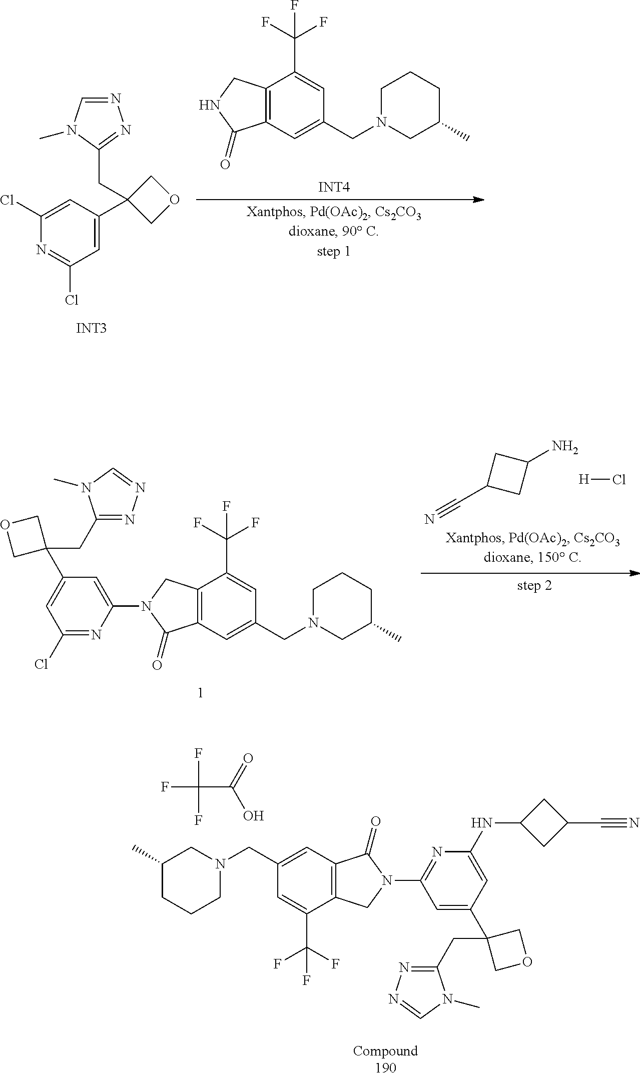

### Step 1: Synthesis of Compound 1

To a stirred mixture of 2,6-dichloro-4-(3-((4-methyl-4H-1,2,4-triazol-3-yl)methyl)oxetan-3-yl)pyridine (235 mg, 0.786 mmol, 1 equiv) and (S)-6-((3-methylpiperidin-1-yl)methyl)-4-(trifluoromethyl)isoindolin-1-one (245 mg, 0.786 mmol, 1 equiv) in dioxane (7 mL) was added Cs₂CO₃ (512 mg, 1.57 mmol, 2 equiv) and Xantphos (90.8 mg, 0.157 mmol, 0.2 equiv) and Pd(OAc)₂ (17.6 mg, 0.0786 mmol, 0.1 equiv) in portions at room temperature. The resulting mixture was stirred for 1 h at 90° C. under nitrogen atmosphere. The mixture was allowed to cool down to room temperature. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (9:1) to afford (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (300 mg, 66.4%) as a green solid. LCMS:(ES·m/z):575,577[M+1]⁺.

### Step 2: Synthesis of Compound 190

To a stirred mixture of (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (30 mg, 0.052 mmol, 1 equiv) and 3-aminocyclobutane-1-carbonitrile hydrochloride (34 mg, 0.26 mmol, 5 equiv) in dioxane (1 mL) was added Cs₂CO₃ (34 mg, 0.1 mmol, 2 equiv), Xantphos (6.0 mg, 0.01 mmol, 0.2 equiv) and Pd(OAc)₂ (1.2 mg, 0.005 mmol, 0.1 equiv) in portions at room temperature. The resulting mixture was stirred for 60 min at 150° C. under nitrogen atmosphere. The reaction was allowed to cool down to room temperature. LCMS indicated the reaction was completed. The mixture was filtered and purified by reversed-phase flash chromatography with the following conditions: column, C18 silica gel; mobile phase, acetonitrile in water (0.1% TFA), 5% to 50% gradient in 30 min; detector, UV 254 nm. The collected fraction was lyophilized to afford (S)-3-((4-(3-((4-methyl-4H-1,2,4-triazol-3-yl)methyl)oxetan-3-yl)-6-(6-((3-methylpiperidin-1-yl)methyl)-1-oxo-4-(trifluoromethyl)isoindolin-2-yl)pyridin-2-yl)amino)cyclobutane-1-carbonitrile; trifluoroacetic acid (8 mg, 24%) as a yellow solid. LCMS (ESI, m/z):635 [M+H−TFA]⁺, 318 [M/2+H−TFA]⁺; ¹H NMR (500 MHz, MeOD) δ 8.54 (s, 1H), 8.22 (s, 1H), 8.17 (s, 1H), 7.38 (s, 1H), 5.92 (s, 1H), 5.30 (s, 2H), 4.99 (s, 4H), 4.51 (s, 3H), 3.97 (s, 2H), 3.27 (s, 2H), 3.53-3.36 (m, 3H), 2.76-2.60 (m, 2H), 2.43 (tdd, J=9.8, 7.6, 2.5 Hz, 2H), 2.02-1.70 (m, 6H), 1.25-1.12 (m, 2H), 1.05 (d, J=4.1 Hz, 3H).

### Synthesis of Compound 191

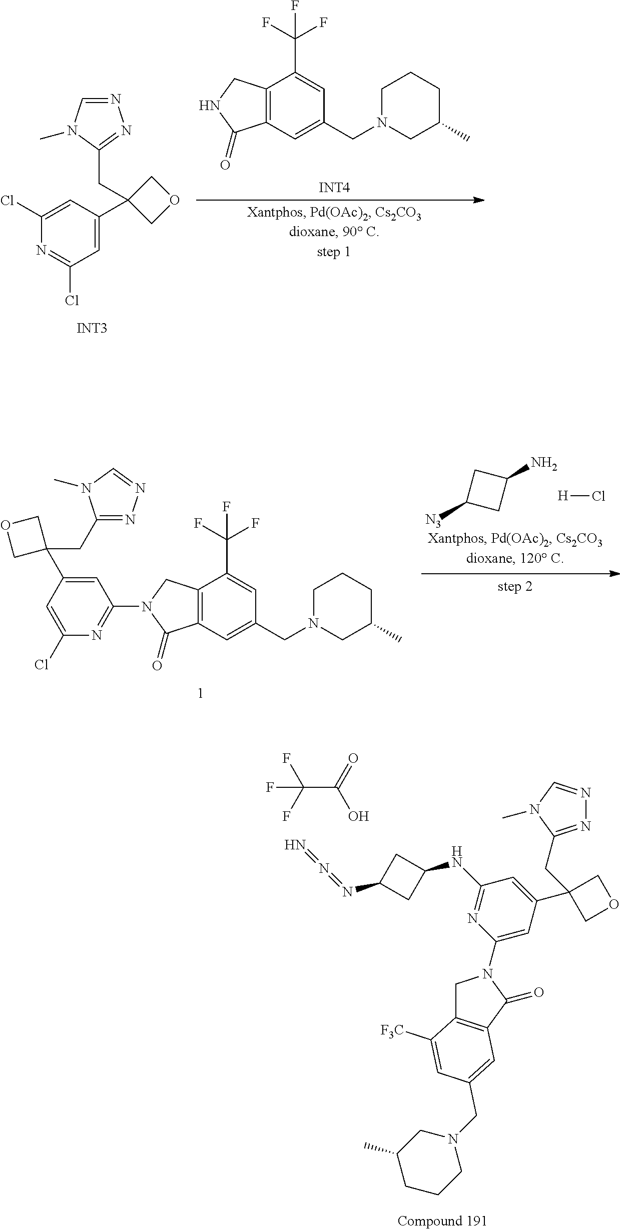

### Step 1: Synthesis of Compound 1

To a stirred mixture of 2,6-dichloro-4-(3-((4-methyl-4H-1,2,4-triazol-3-yl)methyl)oxetan-3-yl)pyridine (235 mg, 0.786 mmol, 1 equiv) and (S)-6-((3-methylpiperidin-1-yl)methyl)-4-(trifluoromethyl)isoindolin-1-one (245 mg, 0.786 mmol, 1 equiv) in dioxane (7 mL) was added Cs₂CO₃ (512 mg, 1.57 mmol, 2 equiv) and Xantphos (90.8 mg, 0.157 mmol, 0.2 equiv) and Pd(OAc)₂ (17.6 mg, 0.0786 mmol, 0.1 equiv) in portions at room temperature. The resulting mixture was stirred for 1 h at 90° C. under nitrogen atmosphere. The mixture was allowed to cool down to room temperature. LCMS indicated the reaction was completed. The reaction was quenched with saturated Sodium bicarbonate at room temperature. The resulting mixture was extracted with CH₂Cl₂ (3×50 ml). The combined organic layers were concentrated under reduced pressure. The residue was purified by silica gel column chromatography, eluted with CH₂Cl₂/MeOH (9:1) to afford (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (300 mg, 66.4%) as a green solid. LCMS:(ES·m/z):575,577[M+1]⁺.

### Step 2: Synthesis of Compound 191A and Compound 191B

To a stirred mixture of (S)-2-(6-chloro-4-(3-((4-methyl-4H-1,2,4-triazol-3-yl)methyl)oxetan-3-yl)pyridin-2-yl)-6-((3-methylpiperidin-1-yl)methyl)-4-(trifluoromethyl)isoindolin-1-one (40 mg, 0.07 mmol, 1 equiv) and (1s,3s)-3-azidocyclobutan-1-amine hydrochloride (31 mg, 0.21 mmol, 3 equiv) in dioxane (1 mL) was added Cs₂CO₃ (46 mg, 0.14 mmol, 2 equiv), Xantphos (8.1 mg, 0.014 mmol, 0.2 equiv) and Pd(OAc)₂ (1.6 mg, 0.007 mmol, 0.1 equiv) in portions at room temperature. The resulting mixture was stirred for 90 min at 120° C. under nitrogen atmosphere. The reaction was allowed to cool down to room temperature. LCMS indicated the reaction was completed. The mixture was filtered and purified by reversed-phase flash chromatography with the following conditions: column, C18 silica gel; mobile phase, acetonitrile in water (0.1% TFA), 5% to 50% gradient in 30 min; detector, UV 254 nm. LCMS (ESI, m/z): 652[M+H−TFA]⁺, 326 [M/2+H−TFA]^{+ 1}H NMR (400 MHz, DMSO) δ 9.53 (s, 1H), 8.24 (m, 2H), 8.17 (s, 1H), 7.46 (s, 1H), 7.08 (s, 1H), 5.93 (s, 1H), 5.20 (s, 2H), 4.91 (s, 1H), 4.89 (s, 3H), 4.77 (d, J=6.1 Hz, 2H), 4.52 (s, 2H), 4.27 (s, 1H), 4.18 (d, J=3.9 Hz, 1H), 3.50 (s, 3H), 3.28 (d, J=8.8 Hz, 2H), 3.24 (s, 4H), 2.85 (s, 1H), 2.59 (d, J=10.6 Hz, 2H), 2.36 (dd, J=7.7, 4.5 Hz, 3H), 1.82 (s, 2H), 1.64 (s, 1H), 1.23 (s, 2H), 1.05 (d, J=13.2 Hz, 2H), 0.88 (d, J=6.4 Hz, 3H).

Then material (7 mg) was purified by Prep-HPLC with the following conditions (Column: XSelect CSH Prep C18 OBD Column, 19*250 mm, 5 m; Mobile Phase A: Water (0.05% TFA), Mobile Phase B: ACN; Flow rate: 25 mL/min; Gradient: 5% B to 95% B in 20 min; wave length: 254 nm. Collected two fractions: LCMS RT (min):

Compound 191A: 1.71 (0.5 mg, 1.1%); LCMS (ESI, m/z): 651[M−TFA]*, 326 [M/2+H−TFA]⁺

Compound 191B: 1.73 (1.2 mg, 2.6%). LCMS (ESI, m/z): 651[M−TFA]⁺, 326 [M/2+H−TFA]⁺

The collected fractions were lyophilized to afford 2-(6-(((1r,3S)-3-(2λ4-triaza-1,2-dien-1-yl)cyclobutyl)amino)-4-(3-((4-methyl-4H-1,2,4-triazol-3-yl)methyl)oxetan-3-yl)pyridin-2-yl)-6-(((S)-3-methylpiperidin-1-yl)methyl)-4-(trifluoromethyl)isoindolin-1-one; trifluoroacetic acid and 2-(6-(((1r,3 S)-3-(2λ4-triaza-1,2-dien-1-yl)cyclobutyl)amino)-4-(3-((4-methyl-4H-1,2,4-triazol-3-yl)methyl)oxetan-3-yl)pyridin-2-yl)-6-(((S)-3-methylpiperidin-1-yl)methyl)-4-(trifluoromethyl)isoindolin-1-one; trifluoroacetic acid.

### Synthesis of Compound 192

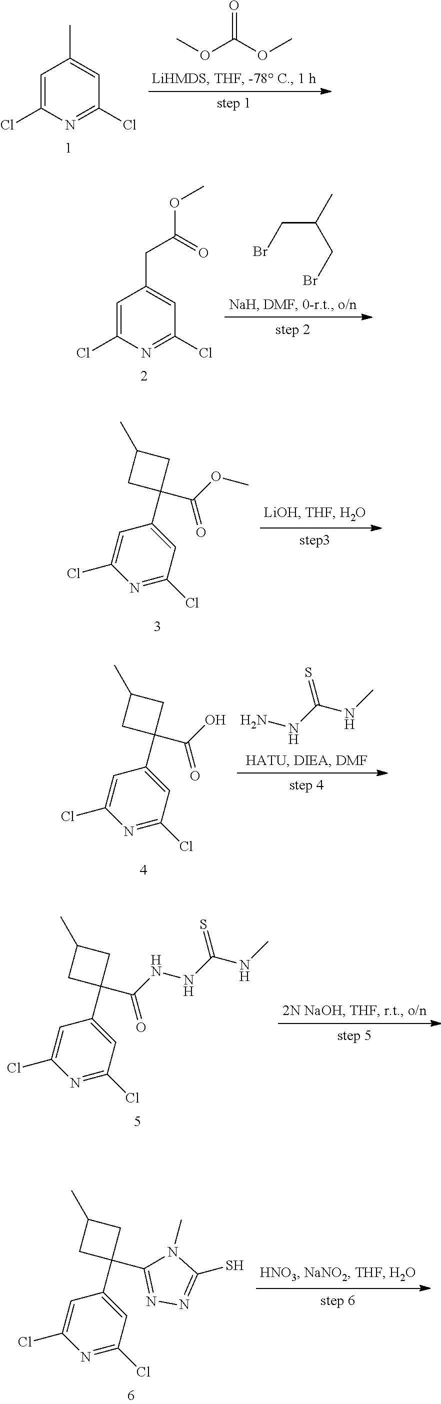

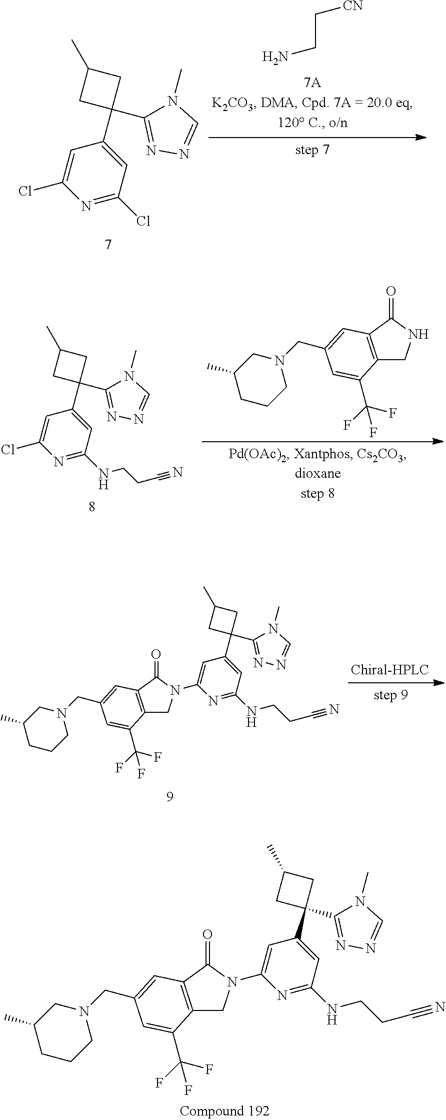 [SMILES: CC1CC(c2cc(Cl)nc(Cl)c2)(c2nncn2C)C1 ; N#CCCN ; CC1CC(c2cc(Cl)nc(NCCC#N)c2)(c2nncn2C)C1 ; C[C@H]1CCCN(Cc2cc3c(c(C(F)(F)F)c2)CNC3=O)C1 ; CC1CC(c2cc(NCCC#N)nc(N3Cc4c(cc(CN5CCC[C@H](C)C5)cc4C(F)(F)F)C3=O)c2)(c2nncn2C)C1 ; C[C@H]1CCCN(Cc2cc3c(c(C(F)(F)F)c2)CN(c2cc([C@]4(c5nncn5C)C[C@@H](C)C4)cc(NCCC#N)n2)C3=O)C1]

### Step 1: Synthesis of Compound 2

To a stirred mixture of 2,6-dichloro-4-methylpyridine (1.0 g, 6.17 mmol, 1.0 equiv) in THE (10 mL) were added LiHMDS (8.02 mL, 8.02 mmol, 1.3 equiv) at −70° C. under nitrogen atmosphere, a mixture of reaction was stirred for 30 min at −70° C. under nitrogen atmosphere. After, to the above mixture was added dimethyl carbonate (1.0 g, 11.10 mmol, 1.80 equiv) at −70° C. under nitrogen atmosphere. The resulting mixture was stirred for 2 h at −70° C. under nitrogen atmosphere. ˜50% desired product could be detected by LCMS. The reaction was quenched with sat. NH₄Cl (aq.) at 0° C. The aqueous layer was extracted with CH₂Cl₂ (3×200 mL). The resulting mixture was concentrated under vacuum. The residue was purified by silica gel column chromatography, eluted with PE/EA (3:1) to afford methyl 2-(2,6-dichloropyridin-4-yl)acetate (590 mg, 41.27%) as an oil. LCMS (ES, m/z): 220, 222 [M+H]⁺.

### Step 2: Synthesis of Compound 3

To a stirred solution of methyl 2-(2,6-dichloropyridin-4-yl)acetate (5.0 g, 22.72 mmol, 1.0 equiv) in DMF (50 mL) were added NaH (2.73 g, 68.16 mmol, 3.00 equiv, 60%) in portions at 0° C. under nitrogen atmosphere. The resulting mixture was stirred for 30 min at 0° C. under nitrogen atmosphere. To the above mixture was added 1,3-dibromo-2-methylpropane (7.36 g, 34.08 mmol, 1.5 equiv) at 0° C. The resulting mixture was stirred for additional 2 h at 10° C. ˜30% desire product could be detected by LCMS. The reaction was quenched with 1 N HCl at room temperature. The mixture was acidified to pH 6 with 1 N HCl The aqueous layer was extracted with CH₂Cl₂ (3×300 mL). The resulting mixture was concentrated under vacuum. The residue was purified by reverse flash chromatography with the following conditions: column, C18 silica gel; mobile phase water (0.1% FA), ACN, 10% to 70% gradient in 30 min; detector, UV 254 nm. The aqueous layer was extracted with CH₂Cl₂ (3×300 mL). The resulting mixture was concentrated under vacuum to afford methyl 1-(2,6-dichloropyridin-4-yl)-3-methylcyclobutane-1-carboxylate (1.9 g, 15.86%) as a yellow oil. LCMS: (ES, m/s): 274, 276 [M+H]⁺.

### Step 3: Synthesis of Compound 4

To a stirred solution of methyl 1-(2,6-dichloropyridin-4-yl)-3-methylcyclobutane-1-carboxylate (1.8 g, 6.56 mmol, 1.0 equiv) in THE (18 mL) were added LiGH (0.31 g, 13.13 mmol, 2.0 equiv) (H₂O=9.0 mL) at room temperature. The resulting mixture was stirred for 1 h at room temperature. LCMS indicated the reaction was completed. The residue was purified by reverse flash chromatography with the following conditions: column, C18 silica gel; mobile phase water (0.1% FA), ACN, 10% to 50% gradient in 30 min; detector, UV 254 nm. The aqueous layer was extracted with CH₂Cl₂ (3×300 mL). The resulting mixture was concentrated under vacuum to afford 1-(2,6-dichloropyridin-4-yl)-3-methylcyclobutane-1-carboxylic acid (2.0 g, 83.14%) as a semi-solid. LCMS: (ES, m/s): 260, 262 [M+H]⁺.

### Step 4: Synthesis of Compound 5

To a stirred solution of 1-(2,6-dichloropyridin-4-yl)-3-methylcyclobutane-1-carboxylic acid (2.4 g, 9.22 mmol, 1.0 equiv) in DMF (24 mL) were added HATU (5.26 g, 13.84 mmol, 1.5 equiv), 1-amino-3-methylthiourea (1.16 g, 11.07 mmol, 1.2 equiv) and DIEA (2.39 g, 18.45 mmol, 2.0 equiv) in portions at 0° C. The resulting mixture was stirred for 2 h at room temperature. LCMS indicated the reaction was completed. The reaction was quenched with water (50 mL) at room temperature. The aqueous layer was extracted with CH₂Cl₂ (3×100 mL). The resulting mixture was concentrated under vacuum. The residue was purified by silica gel column chromatography, eluted with PE/EA (2:3) to afford 1-(2,6-dichloropyridin-4-yl)-3-methyl-N-[(methylcarbamothioyl)amino]cyclobutane-1-carboxamide (2.5 g, 70%) as a white solid. LCMS: (ES, m/s): 347, 349 [M+H]⁺.

### Step 5: Synthesis of Compound 6

To a stirred solution of 1-(2,6-dichloropyridin-4-yl)-3-methyl-N-[(methylcarbamothioyl)amino]cyclobutane-1-carboxamide (2.3 g, 6.62 mmol, 1.0 equiv) in THF (11 mL) were added NaOH (1.0 g, 25.00 mmol, 3.77 equiv) (H₂O=11 mL) in portions at 0° C. under nitrogen atmosphere. The resulting mixture was stirred for overnight at room temperature under nitrogen atmosphere. ˜73% desired product could be detected by LCMS. The mixture was acidified to pH 5 with 1 N HCl. The aqueous layer was extracted with CH₂Cl₂ (3×200 mL). The resulting mixture was concentrated under vacuum. The residue was purified by silica gel column chromatography, eluted with PE/EA (1:1) to afford 5-[1-(2,6-dichloropyridin-4-yl)-3-methylcyclobutyl]-4-methyl-1,2,4-triazole-3-thiol (1.2 g, 55.03%) as a white solid. LCMS: (ES, m/s): 329, 331 [M+H]⁺.

### Step 6: Synthesis of Compound 7

To a stirred solution of 5-[1-(2,6-dichloropyridin-4-yl)-3-methylcyclobutyl]-4-methyl-1,2,4-triazole-3-thiol (1.2 g, 3.64 mmol, 1.0 equiv) in THE (12 mL) were added NaNO₂ (0.75 g, 10.93 mmol, 3.0 equiv) (H₂O=12 mL) and HNO₃ (12 mL, 1.0 mol/L) in portions at 0° C. under nitrogen atmosphere. The resulting mixture was stirred for overnight at room temperature under nitrogen atmosphere. LCMS indicated the reaction was completed. The reaction was quenched with water/ice at room temperature. The aqueous layer was extracted with CH₂Cl₂ (3×200 mL). The resulting mixture was concentrated under vacuum. The residue was purified by reverse flash chromatography with the following conditions: column, silica gel; mobile phase water (0.05% TFA), ACN, 10% to 50% gradient in 30 min; detector, UV 254 nm. The aqueous layer was extracted with CH₂Cl₂ (3×200 mL). The resulting mixture was concentrated under vacuum to afford 2,6-dichloro-4-[3-methyl-1-(4-methyl-1,2,4-triazol-3-yl)cyclobutyl]pyridine (900 mg, 74.78%) as a green solid. LCMS: (ES, m/s): 297, 299 [M+H]⁺.

### Step 7: Synthesis of Compound 8

To a stirred mixture of 2,6-dichloro-4-[3-methyl-1-(4-methyl-1,2,4-triazol-3-yl)cyclobutyl]pyridine (140 mg, 0.47 mmol, 1 equiv) and p aminopropionitrile (660 mg, 9.41 mmol, 19.99 equiv) in DMA (3 mL) was added K₂CO₃ (130 mg, 0.94 mmol, 2.00 equiv) in portions at room temperature under nitrogen atmosphere. The resulting mixture was stirred for overnight at 120° C. under nitrogen atmosphere. LCMS indicated the reaction was completed. The mixture was allowed to cool down to room temperature. The reaction was quenched with water at room temperature. The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with CH₂Cl₂/MeOH (10:1) to afford 3-({6-chloro-4-[3-methyl-1-(4-methyl-1,2,4-triazol-3-yl)cyclobutyl]pyridin-2-yl}amino)propanenitrile (50 mg, 28.87%) as alight yellow solid. LCMS:(ms, ESI): 331, 333[M+H]⁺.

### Step 8: Synthesis of Compound 9

To a stirred mixture of 3-({6-chloro-4-[3-methyl-1-(4-methyl-1,2,4-triazol-3-yl)cyclobutyl]pyridin-2-yl}amino)propanenitrile (50 mg, 0.15 mmol, 1.2 equiv) and 6-{[(3S)-3-methylpiperidin-1-yl]methyl}-4-(trifluoromethyl)-2,3-dihydroisoindol-1-one (40 mg, 0.12 mmol, 1.00 equiv) in dioxane (1 mL) was added Cs₂CO₃ (83 mg, 0.25 mmol, 2 equiv), xantphos (14 mg, 0.02 mmol, 0.2 equiv) and Pd(OAc)₂ (3 mg, 0.01 mmol, 0.1 equiv) under nitrogen atmosphere at room temperature. The resulting mixture was stirred for 2 h at 120° C. under nitrogen atmosphere. LCMS indicated the reaction was completed. The resulting mixture was concentrated under reduced pressure. The residue was purified by reverse flash chromatography with the following conditions: column, C18 silica gel; mobile phase, water (0.1% FA), ACN, 0% to 50% gradient in min; detector, UV 254 nm. to afford 3-({4-[3-methyl-1-(4-methyl-1,2,4-triazol-3-yl)cyclobutyl]-6-(6-{[(3S)-3-methylpiperidin-1-yl]methyl}-1-oxo-4-(trifluoromethyl)-3H-isoindol-2-yl)pyridin-2-yl}amino)propanenitrile (50 mg, 64.35%) as a yellow solid. LCMS:(ES·m/z): 607 [M+H]⁺.

### Step 9: Synthesis of Compound 192

3-({4-[3-methyl-1-(4-methyl-1,2,4-triazol-3-yl)cyclobutyl]-6-(6-{[(3S)-3-methylpiperidin-1-yl]methyl}-1-oxo-4-(trifluoromethyl)-3H-isoindol-2-yl)pyridin-2-yl}amino)propanenitrile (30 mg, 0.049 mmol, 1 equiv) was separated by Chiral-HPLC with the following conditions (Column: CHIRALPAK IE-3, 4.6*50 mm, 3 m; Mobile Phase A: (Hex:DCM=3:1) (0.1% DEA):EtOH=95:5; Flow rate: 1 mL/min; Gradient: 0% B to 0% B; Injection Volume: Sul mL). The eluent of the first peak was concentrated and lyophilized to afford 3-{[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]amino}propanenitrile (7.1 mg, 22.58%) as a white solid. LCMS:(ES·m/z): 607 [M+H]⁺. ¹H NMR (400 MHz, DMSO-d₆) δ 8.35 (s, 1H), 8.01 (s, 2H), 7.76 (s, 1H), 7.17 (s, 1H), 6.26 (s, 1H), 5.24 (s, 2H), 3.54-3.51 (m, 2H), 3.45-3.35 (s, 4H), 3.24 (s, 3H), 2.86-2.70 (m, 6H), 2.60-2.54 (m, 2H), 1.80-1.45 (m, 5H), 1.09 (d, J=4.9 Hz, 3H), 0.92-0.84 (m, 4H).

### Synthesis of Compound 193

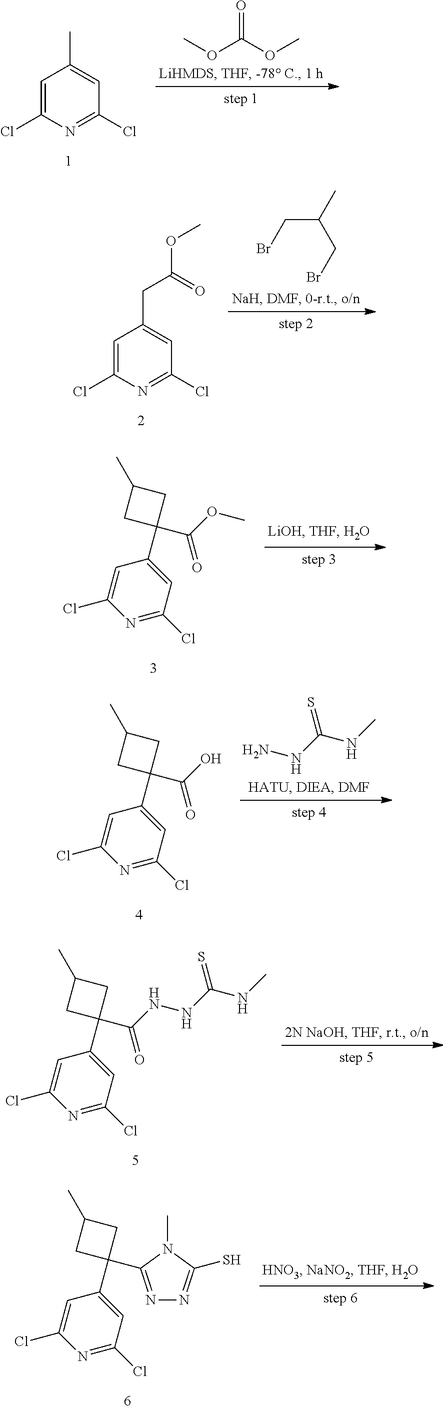

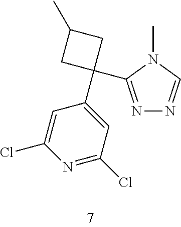 [SMILES: CC1CC(c2cc(Cl)nc(Cl)c2)(c2nncn2C)C1]

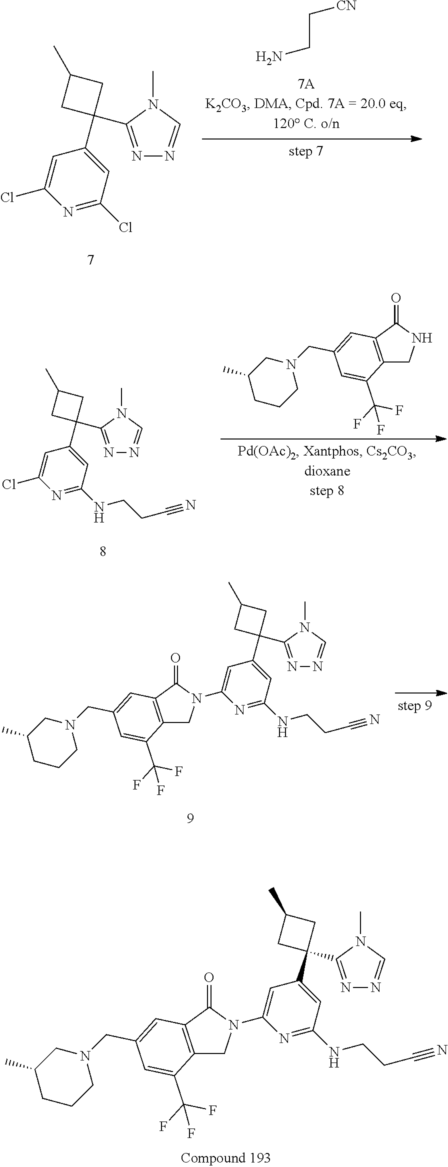 [SMILES: CC1CC(c2cc(Cl)nc(Cl)c2)(c2nncn2C)C1 ; N#CCCN ; CC1CC(c2cc(Cl)nc(NCCC#N)c2)(c2nncn2C)C1 ; C[C@H]1CCCN(Cc2cc3c(c(C(F)(F)F)c2)CNC3=O)C1 ; CC1CC(c2cc(NCCC#N)nc(N3Cc4c(cc(CN5CCC[C@H](C)C5)cc4C(F)(F)F)C3=O)c2)(c2nncn2C)C1 ; C[C@H]1CCCN(Cc2cc3c(c(C(F)(F)F)c2)CN(c2cc([C@]4(c5nncn5C)C[C@H](C)C4)cc(NCCC#N)n2)C3=O)C1]

### Step 1: Synthesis of Compound 2

To a stirred mixture of 2,6-dichloro-4-methylpyridine (1.0 g, 6.17 mmol, 1.0 equiv) in THE (10 mL) were added LiHMDS (8.02 mL, 8.02 mmol, 1.3 equiv) at −70° C. under nitrogen atmosphere, a mixture of reaction was stirred for 30 min at −70° C. under nitrogen atmosphere. After, to the above mixture was added dimethyl carbonate (1.0 g, 11.10 mmol, 1.80 equiv) at −70° C. under nitrogen atmosphere. The resulting mixture was stirred for 2 h at −70° C. under nitrogen atmosphere. ˜50% desired product could be detected by LCMS. The reaction was quenched with sat. NH₄Cl (aq.) at 0° C. The aqueous layer was extracted with CH₂Cl₂ (3×200 mL). The resulting mixture was concentrated under vacuum. The residue was purified by silica gel column chromatography, eluted with PE/EA (3:1) to afford methyl 2-(2,6-dichloropyridin-4-yl)acetate (590 mg, 41.27%) as an oil. LCMS (ES, m/z): 220, 222 [M+H]⁺.

### Step 2: Synthesis of Compound 3

To a stirred solution of methyl 2-(2,6-dichloropyridin-4-yl)acetate (5.0 g, 22.72 mmol, 1.0 equiv) in DMF (50 mL) were added NaH (2.73 g, 68.16 mmol, 3.00 equiv, 60%) in portions at 0° C. under nitrogen atmosphere. The resulting mixture was stirred for 30 min at 0° C. under nitrogen atmosphere. To the above mixture was added 1,3-dibromo-2-methylpropane (7.36 g, 34.08 mmol, 1.5 equiv) at 0° C. The resulting mixture was stirred for additional 2 h at 10° C. ˜30% desire product could be detected by LCMS. The reaction was quenched with Water/HCl at room temperature. The mixture was acidified to pH 6 with 1 N HCl. The aqueous layer was extracted with CH₂Cl₂ (3×300 mL). The resulting mixture was concentrated under vacuum. The residue was purified by reverse flash chromatography with the following conditions: column, C18 silica gel; mobile phase water (0.1% FA), ACN, 10% to 70% gradient in 30 min; detector, UV 254 nm. The aqueous layer was extracted with CH₂Cl₂ (3×300 mL). The resulting mixture was concentrated under vacuum to afford methyl 1-(2,6-dichloropyridin-4-yl)-3-methylcyclobutane-1-carboxylate (1.9 g, 15.86%) as a yellow oil. LCMS: (ES, m/s): 274, 276 [M+H]⁺.

### Step 3: Synthesis of Compound 4

To a stirred solution of methyl 1-(2,6-dichloropyridin-4-yl)-3-methylcyclobutane-1-carboxylate (1.8 g, 6.56 mmol, 1.0 equiv) in THE (18 mL) were added LiGH (0.31 g, 13.13 mmol, 2.0 equiv) (H₂O=9.0 mL) at room temperature. The resulting mixture was stirred for 1 h at room temperature. LCMS indicated the reaction was completed. The residue was purified by reverse flash chromatography with the following conditions: column, C18 silica gel; mobile phase water (0.1% FA), ACN, 10% to 50% gradient in 30 min; detector, UV 254 nm. The aqueous layer was extracted with CH₂Cl₂ (3×300 mL). The resulting mixture was concentrated under vacuum to afford 1-(2,6-dichloropyridin-4-yl)-3-methylcyclobutane-1-carboxylic acid (2.0 g, 83.14%) as a semi-solid. LCMS: (ES, m/s): 260, 262 [M+H]⁺.

### Step 4: Synthesis of Compound 5

To a stirred solution of 1-(2,6-dichloropyridin-4-yl)-3-methylcyclobutane-1-carboxylic acid (2.4 g, 9.22 mmol, 1.0 equiv) in DMF (24 mL) were added HATU (5.26 g, 13.84 mmol, 1.5 equiv), 1-amino-3-methylthiourea (1.16 g, 11.07 mmol, 1.2 equiv) and DIEA (2.39 g, 18.45 mmol, 2.0 equiv) in portions at 0° C. The resulting mixture was stirred for 2 h at room temperature. LCMS indicated the reaction was completed. The reaction was quenched with water (50 mL) at room temperature. The aqueous layer was extracted with CH₂Cl₂ (3×100 mL). The resulting mixture was concentrated under vacuum. The residue was purified by silica gel column chromatography, eluted with PE/EA (2:3) to afford 1-(2,6-dichloropyridin-4-yl)-3-methyl-N-[(methylcarbamothioyl)amino]cyclobutane-1-carboxamide (2.5 g, 70%) as a white solid. LCMS: (ES, m/s): 347, 349 [M+H]⁺.

### Step 5: Synthesis of Compound 6

To a stirred solution of 1-(2,6-dichloropyridin-4-yl)-3-methyl-N-[(methylcarbamothioyl)amino]cyclobutane-1-carboxamide (2.3 g, 6.62 mmol, 1.0 equiv) in THF (11 mL) were added NaOH (1.0 g, 25.00 mmol, 3.77 equiv) (H₂O=11 mL) in portions at 0° C. under nitrogen atmosphere. The resulting mixture was stirred for overnight at room temperature under nitrogen atmosphere. ˜73% desired product could be detected by LCMS. The mixture was acidified to pH 5 with 1 N HCl. The aqueous layer was extracted with CH₂Cl₂ (3×200 mL). The resulting mixture was concentrated under vacuum. The residue was purified by silica gel column chromatography, eluted with PE/EA (1:1) to afford 5-[1-(2,6-dichloropyridin-4-yl)-3-methylcyclobutyl]-4-methyl-1,2,4-triazole-3-thiol (1.2 g, 55.03%) as a white solid. LCMS: (ES, m/s): 329, 331 [M+H]⁺.

### Step 6: Synthesis of Compound 7

To a stirred solution of 5-[1-(2,6-dichloropyridin-4-yl)-3-methylcyclobutyl]-4-methyl-1,2,4-triazole-3-thiol (1.2 g, 3.64 mmol, 1.0 equiv) in THE (12 mL) were added NaNO₂ (0.75 g, 10.93 mmol, 3.0 equiv) (H₂O=12 mL) and HNO₃ (12 mL, 1.0 mol/L) in portions at 0° C. under nitrogen atmosphere. The resulting mixture was stirred for overnight at room temperature under nitrogen atmosphere. LCMS indicated the reaction was completed. The reaction was quenched with water/ice at room temperature. The aqueous layer was extracted with CH₂Cl₂ (3×200 mL). The resulting mixture was concentrated under vacuum. The residue was purified by reverse flash chromatography with the following conditions: column, silica gel; mobile phase water (0.05% TFA), ACN, 10% to 50% gradient in 30 min; detector, UV 254 nm. The aqueous layer was extracted with CH₂Cl₂ (3×200 mL). The resulting mixture was concentrated under vacuum to afford 2,6-dichloro-4-[3-methyl-1-(4-methyl-1,2,4-triazol-3-yl)cyclobutyl]pyridine (900 mg, 74.78%) as a green solid. LCMS: (ES, m/s): 297, 299 [M+H]⁺.

### Step 7: Synthesis of Compound 8

To a stirred mixture of 2,6-dichloro-4-[3-methyl-1-(4-methyl-1,2,4-triazol-3-yl)cyclobutyl]pyridine (140 mg, 0.47 mmol, 1 equiv) and p aminopropionitrile (660 mg, 9.41 mmol, 19.99 equiv) in DMA (3 mL) was added K₂CO₃ (130 mg, 0.94 mmol, 2.00 equiv) in portions at room temperature under nitrogen atmosphere. The resulting mixture was stirred for overnight at 120° C. under nitrogen atmosphere. LCMS indicated the reaction was completed. The mixture was allowed to cool down to room temperature. The reaction was quenched with water at room temperature. The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with CH₂Cl₂/MeOH (10:1) to afford 3-({6-chloro-4-[3-methyl-1-(4-methyl-1,2,4-triazol-3-yl)cyclobutyl]pyridin-2-yl}amino)propanenitrile (50 mg, 28.87%) as alight yellow solid. LCMS:(ms, ESI): 331, 333[M+H]⁺.

### Step 8: Synthesis of Compound 9

To a stirred mixture of 3-({6-chloro-4-[3-methyl-1-(4-methyl-1,2,4-triazol-3-yl)cyclobutyl]pyridin-2-yl}amino)propanenitrile (50 mg, 0.15 mmol, 1.2 equiv) and 6-{[(3S)-3-methylpiperidin-1-yl]methyl}-4-(trifluoromethyl)-2,3-dihydroisoindol-1-one (40 mg, 0.12 mmol, 1.00 equiv) in dioxane (1 mL) was added Cs₂CO₃ (83 mg, 0.25 mmol, 2 equiv), xantphos (14 mg, 0.02 mmol, 0.2 equiv) and Pd(OAc)₂ (3 mg, 0.01 mmol, 0.1 equiv) under nitrogen atmosphere at room temperature. The resulting mixture was stirred for 2 h at 120° C. under nitrogen atmosphere. LCMS indicated the reaction was completed. The resulting mixture was concentrated under reduced pressure. The residue was purified by reverse flash chromatography with the following conditions: column, C18 silica gel; mobile phase, water (0.1% FA), ACN, 0% to 50% gradient in min; detector, UV 254 nm. to afford 3-({4-[3-methyl-1-(4-methyl-1,2,4-triazol-3-yl)cyclobutyl]-6-(6-{[(3S)-3-methylpiperidin-1-yl]methyl}-1-oxo-4-(trifluoromethyl)-3H-isoindol-2-yl)pyridin-2-yl}amino)propanenitrile (50 mg, 64.35%) as a yellow solid. LCMS:(ES·m/z): 607 [M+H]⁺.

### Step 9: Synthesis of Compound 193

3-({4-[3-methyl-1-(4-methyl-1,2,4-triazol-3-yl)cyclobutyl]-6-(6-{[(3S)-3-methylpiperidin-1-yl]methyl}-1-oxo-4-(trifluoromethyl)-3H-isoindol-2-yl)pyridin-2-yl}amino)propanenitrile (30 mg, 0.049 mmol, 1 equiv) was separated by Chiral-HPLC with the following conditions (Column: CHIRALPAK IE-3, 4.6*50 mm, 3 m; Mobile Phase A: (Hex:DCM=3:1) (0.1% DEA):EtOH=95:5; Flow rate: 1 mL/min; Gradient: 0% B to 0% B; Injection Volume: Sul mL). The eluent of the second peak was concentrated and lyophilized to afford 3-{[6-(6-{[(3S)-3-methylpiperidin-1-yl]methyl}-1-oxo-4-(trifluoromethyl)-3H-isoindol-2-yl)-4-[(1s,3r)-3-methyl-1-(4-methyl-1,2,4-triazol-3-yl)cyclobutyl]pyridin-2-yl]amino}propanenitrile (3.1 mg, 10.27%) as a white solid. LCMS:(ES·m/z): 607 [M+H]⁺.

¹H NMR (400 MHz, DMSO-d₆) δ 8.41 (s, 1H), 7.95-7.80 (m, 2H), 7.66 (s, 1H), 7.16-7.13 (m, 1H), 6.09 (s, 1H), 5.20 (s, 2H), 3.64 (s, 2H), 3.52-3.49 (m, 2H), 3.28-3.24 (m, 3H), 3.10-3.05 (m, 2H), 2.84-2.81 (m, 2H), 2.75-2.69 (m, 2H), 2.38-2.30 (m, 1H), 2.20-2.12 (m, 2H), 1.95-1.90 (m, 1H), 1.67-1.58 (m, 4H), 1.52-1.43 (m, 1H), 1.11 (d, J=6.5 Hz, 3H), 0.87-0.81 (m, 4H).

### Synthesis of Compound 194

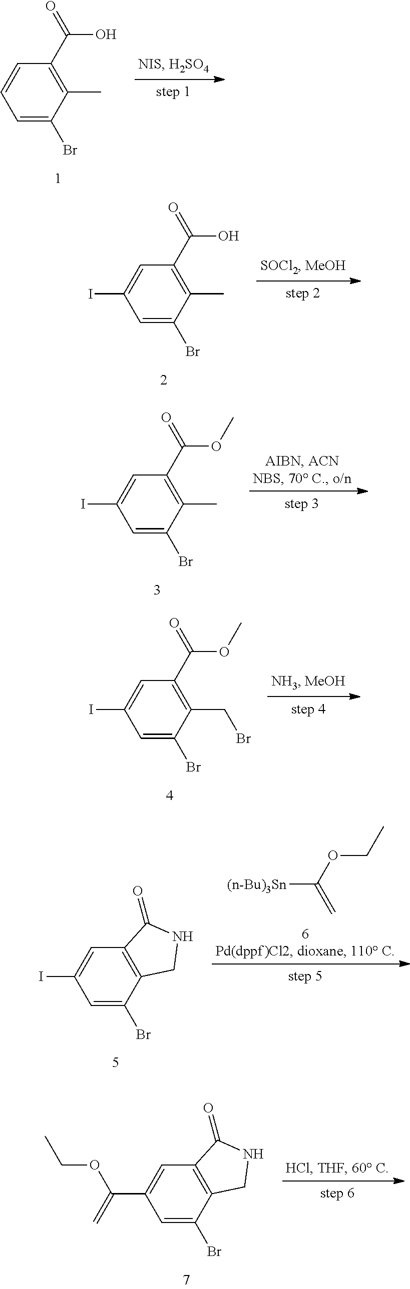

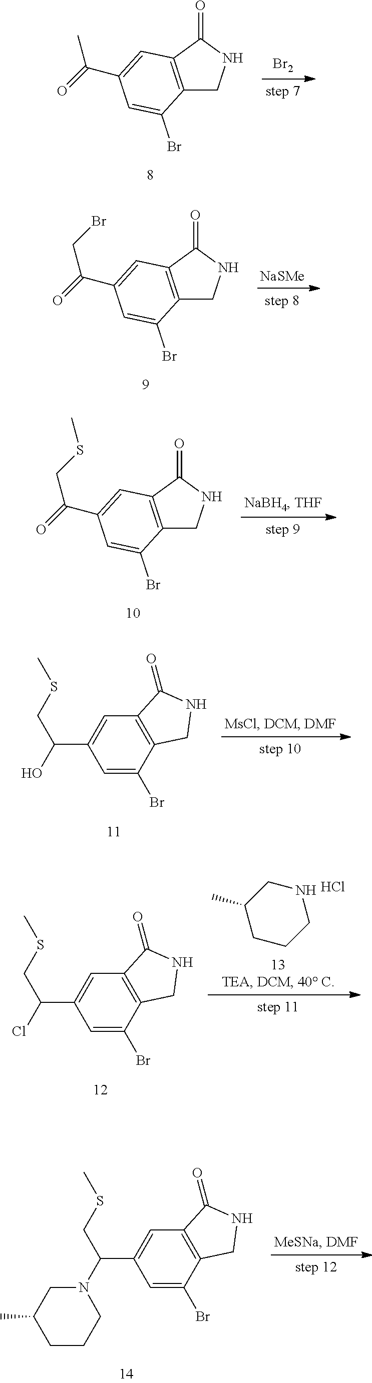

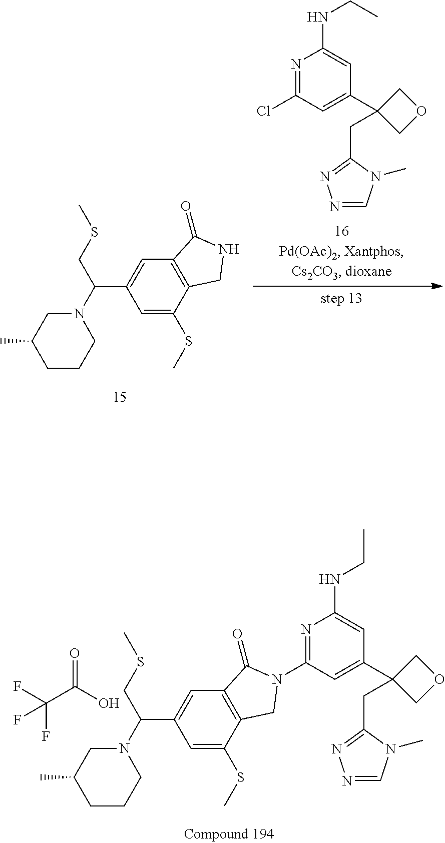

### Step 1: Synthesis of Compound 2

NIS (115.08 g, 511.52 mmol, 1.1 equiv) was added into H2SO₄ (1000 mL) in portions at 0° C. The resulting mixture was stirred for 40 min at 0° C. Then 3-bromo-2-methylbenzoic acid (100 g, 465.02 mmol, 1 equiv) in H2SO₄ (1000 mL) was added into the above mixture and maintain temperature below 5° C. The resulting mixture was stirred for overnight at 25° C. LCMS indicated the reaction was completed. The mixture is poured on crushed ice (3000 g) and the resulting solution is extracted with ethyl acetate (3×2000 mL). The combined organic layers were dried over anhydrous sodium sulfate. After filtration, the filtrate was concentrated under reduced pressure. The residue is triturated with ethyl ether. After filtered and dried, the product 3-bromo-5-iodo-2-methylbenzoic acid (107 g, 67%) is obtained as a white solid. LCMS (ESI, m/z): 339[M−H]⁻; ¹H NMR (300 MHz, DMSO-d₆) δ 8.13 (d, J=1.8 Hz, 1H), 7.99 (d, J=1.8 Hz, 1H), 2.47 (s, 3H).

### Step 2: Synthesis of Compound 3

To a stirred solution of 3-bromo-5-iodo-2-methylbenzoic acid (30.00 g, 88.00 mmol, 1 equiv) in MeOH (300 mL) was added SOCl₂ (52.34 g, 439.96 mmol, 5 equiv) dropwise at 0° C. under nitrogen atmosphere. The resulting mixture was stirred for 3 h at 70° C. under nitrogen atmosphere. LCMS indicated the reaction was completed. The resulting mixture was cooled down to room temperature and concentrated under reduced pressure. The reaction was quenched with sat. NaHCO₃ (aq.) at 0° C. The resulting mixture was extracted with EtOAc (3×100 mL). The combined organic layers were washed with brine (100 mL), dried over anhydrous Na₂SO₄. After filtration, the filtrate was concentrated under reduced pressure. This resulted in methyl 3-bromo-5-iodo-2-methylbenzoate (29.90 g, 95%) as an off-white solid. ¹H NMR (400 MHz, Chloroform-d) δ 8.03 (d, J=2.0 Hz, 1H), 8.02 (d, J=2.0 Hz, 1H), 3.90 (s, 3H), 2.56 (s, 3H).

### Step 3: Synthesis of Compound 4

To a stirred solution of methyl 3-bromo-5-iodo-2-methylbenzoate (30.00 g, 84.51 mmol, 1 equiv) and NBS (18.05 g, 101.42 mmol, 1.2 equiv) in CHCl₃ (300 mL) was added AIBN (6.94 g, 42.26 mmol, 0.5 equiv) in portions at room temperature under nitrogen atmosphere. The resulting mixture was stirred for overnight at 70° C. under nitrogen atmosphere. LCMS indicated the reaction was completed. The resulting mixture was cooled down to room temperature and concentrated under reduced pressure. The residue was purified by silica gel column chromatography, eluted with PE/EA (20:1) to afford methyl 3-bromo-2-(bromomethyl)-5-iodobenzoate (30.00 g, 82%) as a white solid. ¹H NMR (300 MHz, Chloroform-d) δ 8.20 (d, J=1.8 Hz, 1H), 8.11 (d, J=1.8 Hz, 1H), 5.06 (s, 2H), 1.73 (s, 3H).

### Step 4: Synthesis of Compound 5

A solution of methyl 3-bromo-2-(bromomethyl)-5-iodobenzoate (50.30 g, 115.94 mmol, 1 equiv) in NH₃ (g, 7N in MeOH, 750 mL) in portions at 25° C. stirred for 1 h at room temperature under nitrogen atmosphere. Desired product could be detected by LCMS. The resulting mixture was concentrated under reduced pressure. The resulting mixture was washed with (1:1) 800 mL of PE and water. The precipitated solids were collected by filtration and washed with Et₂O (3×380 ml) to afford 4-bromo-6-iodo-2,3-dihydroisoindol-1-one (31.2 g, 80%) as a white solid. LCMS (ESI, ms):338,340[M+H]⁺

### Step 5: Synthesis of Compound 7

To a stirred mixture of 4-bromo-6-iodo-2,3-dihydroisoindol-1-one (5 g, 14.80 mmol, 1.0 equiv) and tributyl(1-ethoxyethenyl)stannane (3.74 g, 10.36 mmol, 0.7 equiv) in dioxane (100 mL) was added Pd(dppf)Cl₂ (3.25 g, 4.44 mmol, 0.3 equiv) in portions at room temperature under nitrogen atmosphere. The resulting mixture was stirred for 1.5 h at 110° C. under nitrogen atmosphere. LCMS indicated the reaction was completed. The mixture was allowed to cool down to room temperature. The resulting mixture was concentrated under reduced pressure. The residue was purified by silica gel column chromatography, eluted with CH₂Cl₂/MeOH (12:1) to afford 4-bromo-6-(1-ethoxyethenyl)-2,3-dihydroisoindol-1-one (2.5 g, 60%) as a brown yellow solid. LCMS (ES·m/z): 282,284 [M+H]⁺.

### Step 6: Synthesis of Compound 8

To a stirred solution of 4-bromo-6-(1-ethoxyethenyl)-2,3-dihydroisoindol-1-one (2.5 g, 8.86 mmol, 1.0 equiv) in THF (50 mL) was added HCl (20 mL) dropwise at 0° C. under air atmosphere. The resulting mixture was stirred for 1 h at 60° C. under air atmosphere. The mixture was allowed to cool down to room temperature. LCMS indicated the reaction was completed. The resulting mixture was concentrated under reduced pressure. The aqueous layer was extracted with EtOAc (3×100 mL). The resulting mixture was concentrated under vacuum. The residue was purified by silica gel column chromatography, eluted with CH₂Cl₂/MeOH (12:1) to afford 6-acetyl-4-bromo-2,3-dihydroisoindol-1-one (1.8 g, 80%) as a brown yellow solid. LCMS:(ES·m/z):254,256 [M+H]⁺.

### Step 7: Synthesis of Compound 9

To a stirred solution of 6-acetyl-4-bromo-2,3-dihydroisoindol-1-one (1.9 g, 7.48 mmol, 1.0 equiv) in CHCl₃ (100 mL) was added Br₂ (1.2 g, 7.59 mmol, 1.0 equiv) at room temperature under nitrogen atmosphere. The reaction was monitored by LCMS. The resulting mixture was stirred for overnight at room temperature under nitrogen atmosphere. Desired product could be detected by LCMS. The resulting mixture was concentrated under reduced pressure. The residue was purified by silica gel column chromatography, eluted with PE/EA (1:1) to afford 4-bromo-6-(2-bromoacetyl)-2,3-dihydroisoindol-1-one (2.0 g, 80%) as a yellow solid. LCMS (ES, m/z): 332,334 [M+H]⁺.

### Step 8: Synthesis of Compound 10

To a stirred solution of 4-bromo-6-(2-bromoacetyl)-2,3-dihydroisoindol-1-one (1.94 g, 5.83 mmol, 1.0 equiv) and (methylsulfanyl)sodium (0.41 g, 5.83 mmol, 1.0 equiv) in MeOH (20 mL) at room temperature under air atmosphere. The resulting mixture was stirred for 1 h at 0° C. under nitrogen atmosphere. The reaction was monitored by LCMS. The resulting mixture was concentrated under reduced pressure. The residue was purified by silica gel column chromatography, eluted with CH₂Cl₂/MeOH (10:1) to afford 4-bromo-6-[2-(methylsulfanyl)acetyl]-2,3-dihydroisoindol-1-one (900 mg, 51%) as a yellow solid. LCMS (ES, m/z): 300,302[M+H]⁺.

### Step 9: Synthesis of Compound 11

To a stirred solution of 4-bromo-6-[2-(methylsulfanyl)acetyl]-2,3-dihydroisoindol-1-one (800 mg, 2.67 mmol, 1.0 equiv) in THE (8 mL) was added NaBH₄ (120 mg, 3.17 mmol, 1.2 equiv) at 0° C. under nitrogen atmosphere. The resulting mixture was stirred for 1 h at 0° C. under nitrogen atmosphere. The reaction was monitored by LCMS. Desired product could be detected by LCMS. The reaction was quenched with MeOH at 0° C. The resulting mixture was concentrated under reduced pressure. The residue was purified by silica gel column chromatography, eluted with CH₂Cl₂/MeOH (10:1) to afford 4-bromo-6-[1-hydroxy-2-(methylsulfanyl)ethyl]-2,3-dihydroisoindol-1-one (410 mg, 51%) as a yellow solid. LCMS (ES, m/z): 302,304 [M+H]⁺.

¹H NMR (400 MHz, DMSO-d₆) δ 8.78 (s, 1H), 7.80 (d, J=1.4 Hz, 1H), 7.69 (d, J=1.4 Hz, 1H), 5.70 (d, J=4.8 Hz, 1H), 4.84 (s, 1H), 4.29 (s, 2H), 2.76 (d, J=6.4 Hz, 2H) 2.04 (s, 3H).

### Step 10: Synthesis of Compound 12

To a stirred solution of 4-bromo-6-[1-hydroxy-2-(methylsulfanyl)ethyl]-2,3-dihydroisoindol-1-one (390 mg, 1.29 mmol, 1.0 equiv) and TEA (273 mg, 2.70 mmol, 2.1 equiv) in DCM (1 mL) was added MsCl (233 mg, 2.0 mmol, 1.6 equiv) dropwise at 0° C. under nitrogen atmosphere. The resulting mixture was stirred for 1 h at 0° C. under nitrogen atmosphere. The reaction was monitored by LCMS. Desired product could be detected by LCMS. The resulting mixture was extracted with EtOAc (3×20 mL). The combined organic layers were washed with brine (10 mL), dried over anhydrous Na₂SO₄. After filtration, the filtrate was concentrated under reduced pressure to afford 4-bromo-6-[1-chloro-2-(methylsulfanyl)ethyl]-2,3-dihydroisoindol-1-one (300 mg, 72%) as a yellow solid. The crude product was used in the next step directly without further purification. LCMS (ES, m/z): 320,322 [M+H]⁺.

### Step 11: Synthesis of Compound 13

To a stirred solution of 4-bromo-6-[1-chloro-2-(methylsulfanyl)ethyl]-2,3-dihydroisoindol-1-one (300 mg, 0.94 mmol, 1.0 equiv) and TEA (189 mg, 1.87 mmol, 2.0 equiv) in DCM (10 mL) was added (3S)-3-methylpiperidine hydrochloride (152 mg, 1.12 mmol, 1.2 equiv) dropwise at 0° C. under nitrogen atmosphere. The resulting mixture was stirred for overnight at 40° C. under nitrogen atmosphere. The reaction was monitored by LCMS. Desired product could be detected by LCMS. The resulting mixture was concentrated under reduced pressure. The residue was purified by reverse phase flash with the following conditions (5-100% ACN in water, 0.05% TFA) to afford 4-bromo-6-{1-[(3S)-3-methylpiperidin-1-yl]-2-(methylsulfanyl)ethyl}-2,3-dihydroisoindol-1-one (160 mg, 44%) as a yellow solid. LCMS (ES, m/z): 383,385 [M+H]⁺.

### Step 12: Synthesis of Compound 15

To a stirred solution of 4-bromo-6-{1-[(3S)-3-methylpiperidin-1-yl]-2-(methylsulfanyl)ethyl}-2,3-dihydroisoindol-1-one (140 mg, 0.37 mmol, 1.0 equiv) and (methylsulfanyl)sodium (31 mg, 0.44 mmol, 1.2 equiv) in DMF (2 mL). The resulting mixture was stirred for 30 min at 90° C. under nitrogen atmosphere. Desired product could be detected by LCMS. The residue was purified by reverse phase flash with the following conditions (5-100% ACN in water, 0.05% TFA) to afford 6-{1-[(3S)-3-methylpiperidin-1-yl]-2-(methylsulfanyl)ethyl}-4-(methylsulfanyl)-2,3-dihydroisoindol-1-one as a yellow solid. LCMS (ES, m/z): 351[M+H]⁺.

### Step 13: Synthesis of Compound 194

To a stirred solution of 6-{1-[(3S)-3-methylpiperidin-1-yl]-2-(methylsulfanyl)ethyl}-4-(methylsulfanyl)-2,3-dihydroisoindol-1-one (130 mg, 0.37 mmol, 1.0 equiv), 6-chloro-N-ethyl-4-{3-[(4-methyl-1,2,4-triazol-3-yl)methyl]oxetan-3-yl}pyridin-2-amine (114 mg, 0.37 mmol, 1.0 equiv), Xantphos (171 mg, 0.30 mmol, 0.8 equiv) and Cs₂CO₃ (362 mg, 1.11 mmol, 3.0 equiv) in dioxane (4 mL) was added Pd(OAc)₂ (33 mg, 0.15 mmol, 0.4 equiv) at room temperature under nitrogen atmosphere. The resulting mixture was stirred for 1 h at 120° C. under nitrogen atmosphere. Desired product could be detected by LCMS. The residue was purified by reversed-phase flash chromatography with the following conditions: column, C18 silica gel; mobile phase, MeCN in Water (0.1% TFA), 5% to 100% gradient in 30 min; detector, UV 254 nm. The crude product was purified by Prep-HPLC with the following conditions (Column: Xselect CSH Prep C18 OBD Column, 19*250 mm, 5 m; Mobile Phase A: Water (0.1% TFA), Mobile Phase B: ACN; Flow rate: 25 mL/min; Gradient: 10% B to 40% B in 10 min; Wave Length: 254 nm/220 nm; RT1 (min): 9.35). The collected fraction was lyophilized to afford 2-[6-(ethylamino)-4-{3-[(4-methyl-1,2,4-triazol-3-yl)methyl]oxetan-3-yl}pyridin-2-yl]-6-{1-[(3S)-3-methylpiperidin-1-yl]-2-(methylsulfanyl)ethyl}-4-(methylsulfanyl)-3H-isoindol-1-one; trifluoroacetic acid (16.5 mg, 6%) as a white solid. LCMS (ES, m/z): 622 [M+H−TFA]⁺; ¹H NMR (400 MHz, DMSO-d₆) δ 8.25 (s, 1H), 7.48 (br s, 1H), 7.38 (s, 1H), 6.58 (t, J=5.4 Hz, 1H), 5.88 (s, 1H), 4.99-4.83 (m, 4H), 4.78 (d, J=6.2 Hz, 2H), 4.0-3.75 (m, 3H), 3.48 (s, 2H), 3.28-3.17 (m, 5H), 3.15-3.05 (m, 2H), 2.9-2.76 (m, 2H), 2.61 (s, 3H), 2.02 (s, 3H), 1.80-1.40 (m, 5H), 1.14 (t, J=7.2 Hz, 3H), 0.80-0.78 (m, 4H).

### Synthesis of Compound 195

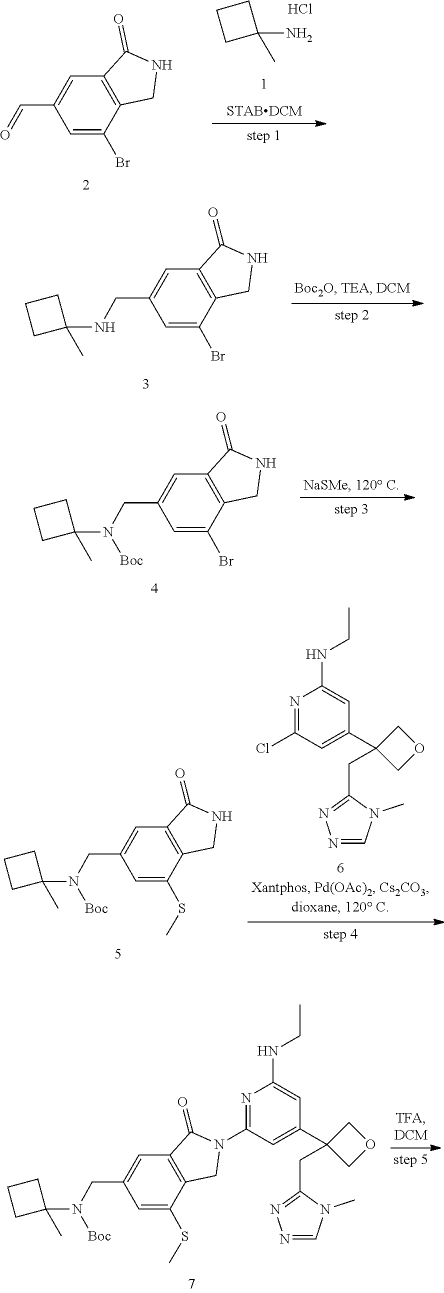

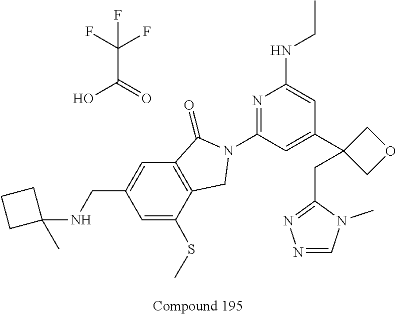

### Step 1: Synthesis of Compound 3

A solution of 7-bromo-3-oxo-1,2-dihydroisoindole-5-carbaldehyde (3 g, 12.50 mmol, 1.0 equiv) in DCM (30 mL) was treated with 1-methylcyclobutan-1-amine hydrochloride (2.28 g, 18.75 mmol, 1.5 equiv) and TEA (3.79 g, 37.50 mmol, 3.0 equiv). The reaction mixture was stirred for 30 min at room temperature. To the above mixture was added STAB (7.95 g, 37.50 mmol, 3.0 equiv) in portions. The resulting mixture was stirred for additional overnight at room temperature. Desired product could be detected by LCMS. The resulting mixture was concentrated under reduced pressure. The residue was purified by silica gel column chromatography, eluted with CH₂Cl₂/MeOH (10:1) to afford 4-bromo-6-{[(1-methylcyclobutyl)amino]methyl}-2,3-dihydroisoindol-1-one (3.6 g, 74%) as a white solid. LCMS (ES, m/z): 309,311 [M+H]⁺.

### Step 2: Synthesis of Compound 4

A solution of 4-bromo-6-{[(1-methylcyclobutyl)amino]methyl}-2,3-dihydroisoindol-1-one (3.60 g, 11.64 mmol, 1.0 equiv) in THE (36 mL) was basified to pH=9 with NaHCO₃ (sat.aq) and the mixture was treated with di-tert-butyl dicarbonate (3.05 g, 13.97 mmol, 1.2 equiv). The resulting mixture was stirred for 16 h at room temperature. Desired product could be detected by LCMS. The resulting mixture was concentrated under reduced pressure. The residue was purified by silica gel column chromatography, eluted with CH₂Cl₂/MeOH (10:1) to afford tert-butyl N-[(7-bromo-3-oxo-1,2-dihydroisoindol-5-yl)methyl]-N-(1-methylcyclobutyl)carbamate (2.70 g, 49%) as a white solid. LCMS (ES, m/z): 409,411 [M+H]⁺.

### Step 3: Synthesis of Compound 5

A solution of tert-butyl N-[(7-bromo-3-oxo-1,2-dihydroisoindol-5-yl)methyl]-N-(1-methylcyclobutyl)carbamate (2.0 g, 4.89 mmol, 1.0 equiv) in DMF (10 mL) was treated with NaSMe (0.68 g, 9.77 mmol, 2.0 equiv). The resulting mixture was stirred for 1 h at 120° C. under nitrogen atmosphere. Desired product could be detected by LCMS. The reaction mixture was allowed to cool down to room temperature and quenched with water at room temperature. The resulting mixture was extracted with EtOAc (3×10 mL). The combined organic layers were washed with water (10 mL), dried over anhydrous Na₂SO₄. After filtration, the filtrate was concentrated under reduced pressure. The residue was purified by silica gel column chromatography, eluted with CH₂Cl₂/MeOH (10:1) to afford tert-butyl N-(1-methylcyclobutyl)-N-{[7-(methylsulfanyl)-3-oxo-1,2-dihydroisoindol-5-yl]methyl}carbamate (1.90 g, 87%) as an off-white solid. LCMS (ES, m/z): 377 [M+H]⁺.

### Step 4: Synthesis of Compound 7

A solution of tert-butyl N-(1-methylcyclobutyl)-N-{[7-(methylsulfanyl)-3-oxo-1,2-dihydroisoindol-5-yl]methyl}carbamate (400 mg, 1.06 mmol, 1.0 equiv) in dioxane (5 mL) was treated with 6-chloro-N-ethyl-4-{3-[(4-methyl-1,2,4-triazol-3-yl)methyl]oxetan-3-yl}pyridin-2-amine (327 mg, 1.06 mmol, 1.0 equiv), Pd(OAc)₂ (48 mg, 0.21 mmol, 0.2 equiv), [5-(diphenylphosphanyl)-9,9-dimethyl-9H-xanthen-4-yl]diphenylphosphane (246 mg, 0.43 mmol, 0.4 equiv) and Cs₂CO₃ (692 mg, 2.12 mmol, 2.0 equiv) under nitrogen atmosphere. The resulting mixture was stirred for 1.5 h at 120° C. under nitrogen atmosphere. Desired product could be detected by LCMS. The reaction mixture was allowed to cool down to room temperature and concentrated to dryness under vacuum. The residue was purified by silica gel column chromatography, eluted with CH₂Cl₂/MeOH (10:1) to afford tert-butyl N-({2-[6-(ethylamino)-4-{3-[(4-methyl-1,2,4-triazol-3-yl)methyl]oxetan-3-yl}pyridin-2-yl]-7-(methylsulfanyl)-3-oxo-1H-isoindol-5-yl}methyl)-N-(1-methylcyclobutyl)carbamate (190 mg, 22%) as an off-white solid.

LCMS (ES, m/z): 648 [M+H]⁺.

### Step 5: Synthesis of Compound 195

A solution of tert-butyl N-({2-[6-(ethylamino)-4-{3-[(4-methyl-1,2,4-triazol-3-yl)methyl]oxetan-3-yl}pyridin-2-yl]-7-(methylsulfanyl)-3-oxo-1H-isoindol-5-yl}methyl)-N-(1-methylcyclobutyl)carbamate (90 mg, 0.14 mmol, 1.0 equiv) in DCM (3 mL) was treated with TFA (1 mL). The resulting mixture was stirred for 1 h at room temperature. Desired product could be detected by LCMS. The reaction mixture was concentrated to dryness under vacuum. The residue was submitted to Prep-HPLC purification (Column: Xselect CSH Prep C18 Column, 30*150 mm, 5 m; Mobile Phase A: Water (0.05% TFA), Mobile Phase B: ACN; Flow rate: 60 mL/min mL/min; Gradient: 10% B to 40% B in 10 min; Wave Length: 254 nm/220 nm; RT1 (min): 8.68) and the collected fractions were lyophilized to afford 2-[6-(ethylamino)-4-{3-[(4-methyl-1,2,4-triazol-3-yl)methyl]oxetan-3-yl}pyridin-2-yl]-6-{[(1-methylcyclobutyl)amino]methyl}-4-(methylsulfanyl)-3H-isoindol-1-one; trifluoroacetic acid (25.50 mg, 27%) as a yellow solid. LCMS (ESI, m/z): 548 [M+H]⁺. ¹H NMR (400 MHz, DMSO-d₆) δ 9.30-8.88 (m, 3H), 7.74 (s, 2H), 7.36 (s, 1H), 6.00 (m, 1H), 4.94-4.77 (m, 6H), 4.27-4.21 (m, 2H), 3.66 (s, 2H), 3.41 (s, 3H), 3.27-3.22 (m, 2H), 2.63 (s, 3H), 2.47-2.43 (m, 2H), 1.96-1.86 (m, 4H), 1.57 (s, 3H), 1.14 (t, J=7.2 Hz, 3H).

### Synthesis of Compound 196

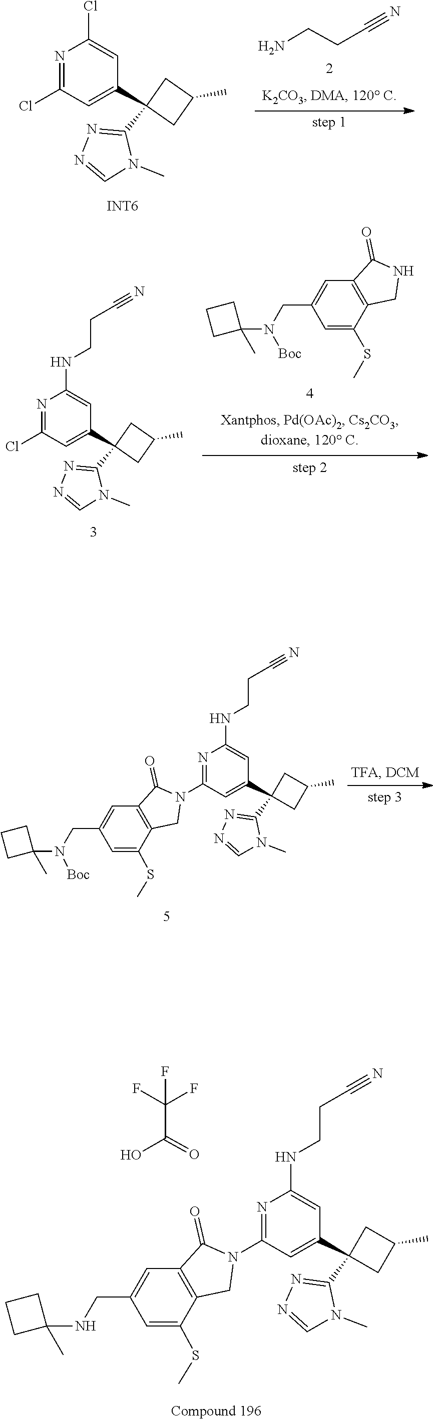

### Step 1: Synthesis of Compound 3

A solution of 2,6-dichloro-4-[(1r,3s)-3-methyl-1-(4-methyl-1,2,4-triazol-3-yl)cyclobutyl]pyridine (500 mg, 1.68 mmol, 1.0 equiv) in DMA (10 mL) was treated with O-aminopropionitrile (2.36 g, 33.64 mmol, 20.0 equiv), and K₂CO₃ (465 mg, 3.36 mmol, 2.0 equiv). The resulting mixture was stirred for 16 h at 120° C. under nitrogen atmosphere. Desired product could be detected by LCMS. The reaction mixture was allowed to cool down to room temperature and purified by reverse flash chromatography with the following conditions: column, C₁₈, 40 g, 20-35 um; mobile phase, water with 0.08% NH₄HCO₃ and ACN (0% to 50% gradient in 50 min); detector, UV 254 nm. The collected fractions were lyophilized to afford 3-({6-chloro-4-[(1r,3s)-3-methyl-1-(4-methyl-1,2,4-triazol-3-yl)cyclobutyl]pyridin-2-yl}amino)propanenitrile (310 mg, 48%) as an off-white solid. LCMS (ESI, m/z): 331,333 [M+H]⁺.

### Step 2: Synthesis of Compound 5

A solution of 3-({6-chloro-4-[(1r,3s)-3-methyl-1-(4-methyl-1,2,4-triazol-3-yl)cyclobutyl]pyridin-2-yl}amino)propanenitrile (200 mg, 0.61 mmol, 1.0 equiv) in dioxane (12 mL) was treated with tert-butyl N-(1-methylcyclobutyl)-N-{[7-(methylsulfanyl)-3-oxo-1,2-dihydroisoindol-5-yl]methyl}carbamate (341 mg, 0.91 mmol, 1.5 equiv), Pd(OAc)₂ (54 mg, 0.24 mmol, 0.4 equiv), [5-(diphenylphosphanyl)-9,9-dimethyl-9H-xanthen-4-yl]diphenylphosphane (140 mg, 0.24 mmol, 0.4 equiv) and Cs₂CO₃ (1.58 g, 4.84 mmol, 8.0 equiv). The resulting mixture was stirred for 1.5 h at 100° C. under nitrogen atmosphere. Desired product could be detected by LCMS. The resulting mixture was concentrated under reduced pressure. The residue was purified by silica gel column chromatography, eluted with CH₂Cl₂/MeOH (20:1) to afford tert-butyl N-[(2-{6-[(2-cyanoethyl)amino]-4-[(1r,3s)-3-methyl-1-(4-methyl-1,2,4-triazol-3-yl)cyclobutyl]pyridin-2-yl}-7-(methylsulfanyl)-3-oxo-1H-isoindol-5-yl)methyl]-N-(1-methylcyclobutyl)carbamate (120 mg, 26%) as an off-white solid. LCMS (ESI, m/z): 671 [M+H]⁺.

### Step 3. Synthesis of Compound 196

A solution of tert-butyl N-[(2-{6-[(2-cyanoethyl)amino]-4-[(1r,3s)-3-methyl-1-(4-methyl-1,2,4-triazol-3-yl)cyclobutyl]pyridin-2-yl}-7-(methylsulfanyl)-3-oxo-1H-isoindol-5-yl)methyl]-N-(1-methylcyclobutyl)carbamate (40 mg, 0.060 mmol, 1.0 equiv) in DCM (3 mL) was treated with TFA (1 mL). The resulting mixture was stirred for 1 h at room temperature. Desired product could be detected by LCMS. The resulting mixture was stirred for 1 h at room temperature. Desired product could be detected by LCMS. The residue was submitted to Prep-HPLC purification (Column: XBridge Prep OBD C₁₈ Column, 19*250 mm, 5 m; Mobile Phase A: water (0.05% TFA), Mobile Phase B: ACN; Flow rate: 25 mL/min mL/min; Gradient: 7% B to 47% B in 10 min; Wave Length: 254 nm/220 nm; RT1 (min): 9.77) and the collected fractions were lyophilized to afford 3-{[6-(6-{[(1-methylcyclobutyl)amino]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]amino}propanenitrile; trifluoroacetic acid (20.40 mg, 49%) as a white solid. LCMS (ESI, m/z): 571 [M+H−TFA]⁺. ¹H NMR (400 MHz, DMSO-d₆) δ 9.20 (br s, 1H), 8.60 (s, 1H), 7.74 (d, J=2.8 Hz, 3H), 7.34-6.99 (m, 1H), 6.30 (s, 1H), 4.97 (s, 2H), 4.22-4.20 (m, 2H), 3.56 (d, J=5.6 Hz, 2H), 3.30 (s, 3H), 2.86 (t, J=7.6 Hz, 2H), 2.79-2.77 (m, 2H), 2.64 (s, 3H), 2.57-2.53 (m, 3H), 2.47-2.40 (m, 2H), 1.92-1.84 (m, 4H), 1.57 (s, 3H), 1.10 (d, J=4.8 Hz, 3H).

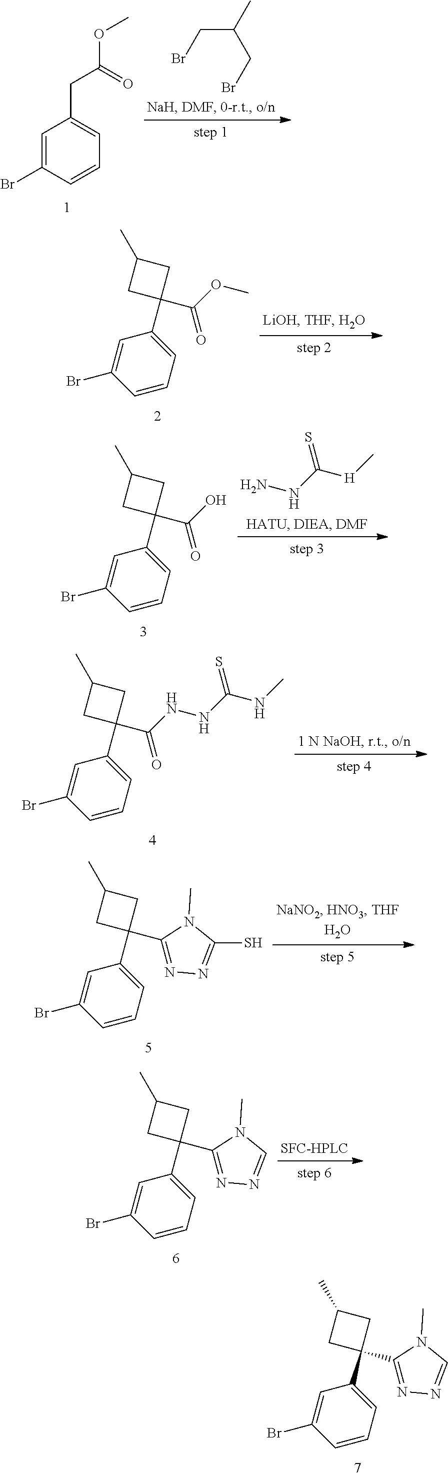

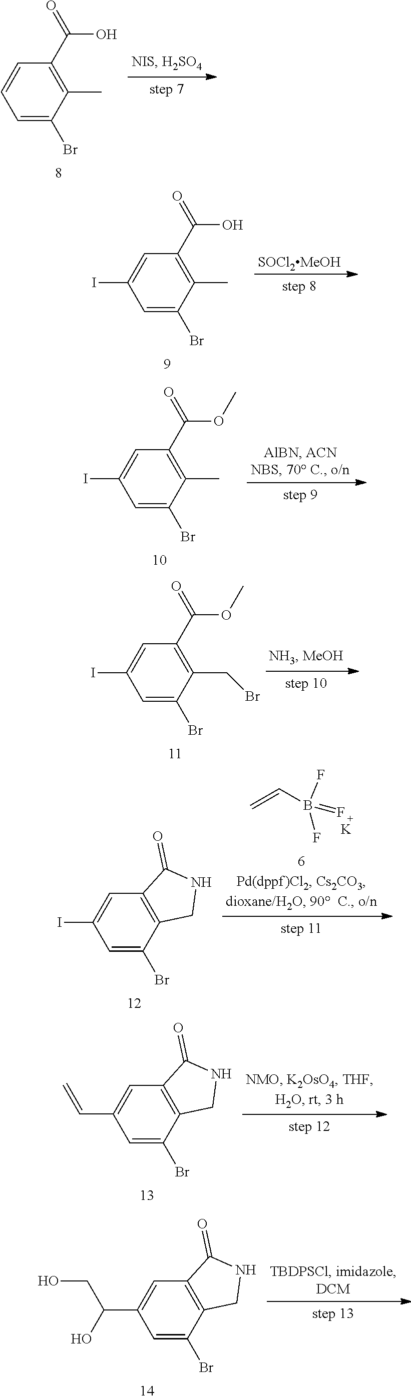

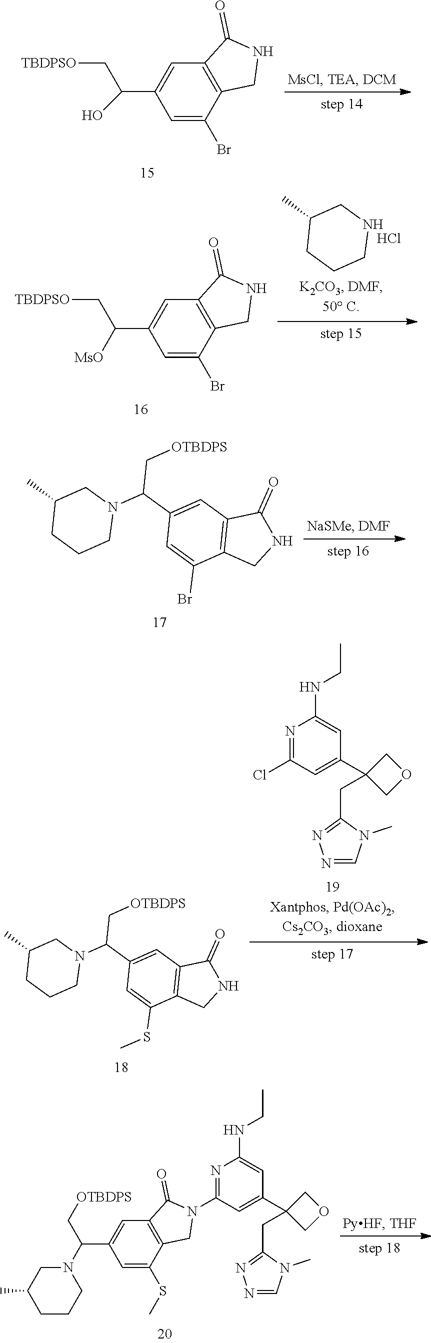

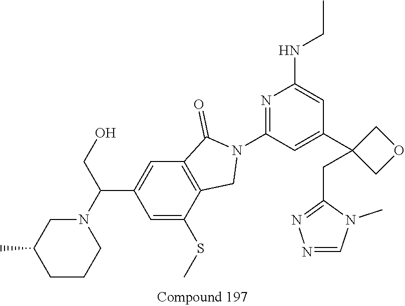 [SMILES: CCNc1cc(C2(Cc3nncn3C)COC2)cc(N2Cc3c(SC)cc(C(CO)N4CCC[C@H](C)C4)cc3C2=O)n1]

### Synthesis of Compound 197

### Step 1. Synthesis of Compound 2

To a stirred solution of methyl 2-(3-bromophenyl)acetate (100 g, 436.54 mmol, 1 equiv) in DMF (1 L) was added NaH (26.19 g, 1091.35 mmol, 1.5 equiv, 60%) in portions at 0° C. under air atmosphere. The resulting mixture was stirred for 30 min at 0° C. under air atmosphere. To the above mixture was added 1,3-dibromo-2-methylpropane (103.68 g, 480.19 mmol, 1.1 equiv) in portions over at 0° C. The resulting mixture was stirred for additional overnight at room temperature. LCMS indicated the reaction was completed. The reaction was quenched by the addition of sat. NH₄Cl (aq.) (1000 mL) at 0° C. The resulting mixture was extracted with EtOAc (3×700 mL). The combined organic layers were washed with brine (3×200 mL), dried over anhydrous Na₂SO₄. After filtration, the filtrate was concentrated under reduced pressure. The residue was purified by silica gel column chromatography, eluted with PE/EA (5:1) to afford methyl 1-(3-bromophenyl)-3-methylcyclobutane-1-carboxylate (100 g, 81%) as a yellow oil. LCMS:(ES·m/z):283,284[M+H]⁺.

### Step 2: Synthesis of Compound 3

To a stirred solution of methyl 1-(3-bromophenyl)-3-methylcyclobutane-1-carboxylate (100 g, 353.15 mmol, 1 equiv) in THE (100 mL) was added LiGH (25.37 g, 1059.45 mmol, 3 equiv) in H₂O (1000 mL) at 0° C. The resulting mixture was stirred for overnight at room temperature under nitrogen atmosphere. LCMS indicated the reaction was completed. The resulting mixture was concentrated under vacuum. The mixture was acidified to pH 6 with conc. HCl. The resulting mixture was extracted with EtOAc (3×200 mL). The combined organic layers were washed with brine (3×100 mL), dried over anhydrous Na₂SO₄. After filtration, the filtrate was concentrated under reduced pressure. This resulted in 1-(3-bromophenyl)-3-methylcyclobutane-1-carboxylic acid (30 g, 32%) as a yellow solid. LCMS (ESI, m/z):267,269[M−H]⁻.

### Step 3: Synthesis of Compound 4

To a stirred solution of 1-(3-bromophenyl)-3-methylcyclobutane-1-carboxylic acid (16.5 g, 61.31 mmol, 1 equiv) and 1-amino-3-methylthiourea (7.74 g, 73.57 mmol, 1.2 equiv) in DMF (170 mL) was added HATU (34.97 g, 91.96 mmol, 1.5 equiv) and DIEA (15.85 g, 122.61 mmol, 2 equiv) at room temperature. The resulting mixture was stirred for overnight at room temperature under nitrogen atmosphere. LCMS indicated the reaction was completed. The reaction mixture was used to next step without any treatment. LCMS:(ES·m/z):356,358[M+H]⁺.

### Step 4: Synthesis of Compound 5

To the above mixture was added NaOH (170 mL) at room temperature. The resulting mixture was stirred for additional overnight at room temperature. LCMS indicated the reaction was completed. The mixture was acidified to pH 6 with 1N HCl. The resulting mixture was extracted with EtOAc (3×100 mL). The combined organic layers were washed with brine (5×100 mL), dried over anhydrous Na₂SO₄. After filtration, the filtrate was concentrated under reduced pressure. The residue was purified by silica gel column chromatography, eluted with CH₂Cl₂/MeOH (10:1) to afford 5-[1-(3-bromophenyl)-3-methylcyclobutyl]-4-methyl-1,2,4-triazole-3-thiol (4.1 g, 20%) as a yellow oil. LCMS:(ES·m/z):338,340[M+H]⁺.

### Step 5: Synthesis of Compound 6

To a stirred mixture of 5-[1-(3-bromophenyl)-3-methylcyclobutyl]-4-methyl-1,2,4-triazole-3-thiol (22 g, 65.03 mmol, 1.00 equiv) in THE (22.02 mL) was added NaNO₂ (22.44 g, 325.18 mmol, 5 equiv) in portions at 0 degrees C. under nitrogen atmosphere. The mixture was added HNO₃ (325.18 mL, 325.18 mmol, 5 equiv, 1N) dropwise at 0 degrees C. under nitrogen atmosphere. LCMS indicated the reaction was completed. The mixture was basified to pH 8 with saturated NaHCO₃ (aq.). The resulting mixture was extracted with EtOAc (3×200 mL). The combined organic layers were washed with water (4×80 mL). The resulting mixture was concentrated under reduced pressure. The residue was purified by silica gel column chromatography, eluted with CH₂Cl₂/MeOH (10:1) to afford 3-[1-(3-bromophenyl)-3-methylcyclobutyl]-4-methyl-1,2,4-triazole (10 g, 50%) as an off-white solid. LCMS:(ES·m/z):306,308[M+H]⁺.

### Step 6: Synthesis of Compound 7

The product (10 g) was separated by Prep-SFC with the following conditions (Column: CHIRALPAK ID 2*25 cm, Sum; Mobile Phase A: CO₂, Mobile Phase B: IPA (1%-2M-NH₃-MeOH); Flow rate: 55 mL/min; Gradient: isocratic 20% B; Column Temperature (° C.): 35; Back Pressure (bar): 100; Wave Length: 220 nm; RT1 (min): 12.13; RT2 (min): 13.8; Sample Solvent: MEOH; Injection Volume: 0.8 mL. The second fraction (RT13.8 min) was concentrated to afford 4-methyl-3-[(1r,3s)-1-(3-bromophenyl)-3-methylcyclobutyl]-1,2,4-triazole (6 g, 60%) as an off-white solid. ¹H NMR (300 MHz, Chloroform-d) δ 8.00 (s, 1H), 7.53 (s, 1H), 7.40-7.37 (s, 1H), 7.24-7.21 (m, 2H), 3.19 (s, 3H), 2.82-2.78 (m, 2H), 2.67-2.63 (m, 3H), 1.22-1.12 (m, 3H).

### Step 7: Synthesis of Compound 9

To a stirred solution of NIS (57.54 g, 255.76 mmol, 1.1 equiv) in H2SO₄ (200 mL) was stirred for 40 min at room temperature under nitrogen atmosphere. To the above mixture was added 3-bromo-2-methylbenzoic acid (50 g, 232.51 mmol, 1 equiv) in H2SO₄ (200 mL) dropwise at 0° C. and stirred for 3 h, The resulting mixture was stirred for additional overnight at room temperature. LCMS indicated complete reaction. LCMS indicated complete reaction. The reaction was pour into Water/Ice at 0° C. The precipitated solids were collected by filtration and washed with Et₂O (3×20 mL). The residue was purified by trituration with DCM (100 mL). The precipitated solids were collected by filtration and washed with DCM (3×50 mL). The solid was dried under infrared to afford 3-bromo-5-iodo-2-methylbenzoic acid (54.2 g, 68%) as an off-white solid. LCMS (ESI, ms):339,341[M−H]⁻. ¹H NMR (300 MHz, DMSO-d₆) δ 8.13 (d, J=1.8 Hz, 1H), 7.99 (d, J=1.8 Hz, 1H), 2.47 (s, 3H).

### Step 8: Synthesis of Compound 10

To a stirred solution of 3-bromo-5-iodo-2-methylbenzoic acid (30 g, 87.99 mmol, 1 equiv) in MeOH (300 mL) was added SOCl₂ (52.34 g, 439.96 mmol, 5 equiv) dropwise at 0° C. under nitrogen atmosphere. The resulting mixture was stirred for 3 h at 70° C. under nitrogen atmosphere. LCMS indicated the reaction was completed. The resulting mixture was concentrated under reduced pressure. The reaction was quenched with sat. NaHCO₃ (aq.) at 0° C. The resulting mixture was extracted with EtOAc (3×100 mL). The combined organic layers were washed with brine (100 mL), dried over anhydrous Na₂SO₄. After filtration, the filtrate was concentrated under reduced pressure. This resulted in methyl 3-bromo-5-iodo-2-methylbenzoate (29.9 g, 96%) as an off-white solid. No MS signal. ¹H NMR (400 MHz, Chloroform-d) δ 8.03 (d, J=1.9 Hz, 1H), 8.02 (d, J=1.9 Hz, 1H), 3.90 (s, 3H), 2.56 (s, 3H).

### Step 9: Synthesis of Compound 11

To a stirred solution of methyl 3-bromo-5-iodo-2-methylbenzoate (15 g, 42.25 mmol, 1 equiv) and NBS (9.03 g, 50.70 mmol, 1.2 equiv) in ACN (150 mL) was added AIBN (3.47 g, 21.12 mmol, 0.5 equiv) in portions at 70° C. under nitrogen atmosphere. LCMS & TLC showed the reaction was completed. no MS signal. The resulting mixture was concentrated under reduced pressure. The residue was purified by silica gel column chromatography, eluted with PE/EA (6:1) to afford methyl 3-bromo-2-(bromomethyl)-5-iodobenzoate (16.5 g, 90%) as a white solid. ¹H NMR (300 MHz, Chloroform-d) δ 8.20 (d, J=1.9 Hz, 1H), 8.11 (d, J=1.8 Hz, 1H), 5.06 (s, 2H), 1.73 (s, 3H).

### Step 10: Synthesis of Compound 12

A mixture of methyl 3-bromo-2-(bromomethyl)-5-iodobenzoate (51.5 g, 118.70 mmol, 1 equiv) and NH₃ (7N in MeOH, 500 mL) was stirred for 2 h at 25° C. LCMS indicated the reaction was completed. The solvent was removed under reduced pressure. The residue was used for next step without further purification. LCMS (ESI, ms): 338,340 [M+H]⁺.

### Step 11: Synthesis of Compound 13

To a stirred solution of 4-bromo-6-iodo-2,3-dihydroisoindol-1-one (48 g, 142.03 mmol, 1 equiv) and potassium ethenyltrifluoroboranuide (15.22 g, 113.62 mmol, 0.8 equiv) in 1,4-dioxane (500 mL) and H₂O (100 mL) was added Cs₂CO₃ (92.56 g, 284.07 mmol, 2 equiv) and Pd(dppf)Cl₂ (10.39 g, 14.20 mmol, 0.1 equiv) at room temperature under nitrogen atmosphere. The resulting mixture was stirred for 1 h at 90° C. under nitrogen atmosphere. LCMS indicated the reaction was completed. The mixture was allowed to cool down to room temperature. The resulting mixture was concentrated under reduced pressure. The residue was purified by silica gel column chromatography, eluted with CH₂Cl₂/MeOH (12:1) to afford 4-bromo-6-ethenyl-2,3-dihydroisoindol-1-one (11 g, 32%) as a brown solid. LCMS (ESI, ms):238,240[M+H]⁺.

### Step 12: Synthesis of Compound 14

To a stirred solution of 4-bromo-6-ethenyl-2,3-dihydroisoindol-1-one (20 g, 84.00 mmol, 1 equiv) and NMO (29.52 g, 252.00 mmol, 3.00 equiv) in THE (200 mL) and water (100 mL) was added K₂OsO₄·2H₂O (0.15 g, 0.42 mmol, 0.05 equiv) at room temperature under nitrogen atmosphere. The resulting mixture was stirred for 3 h at room temperature under nitrogen atmosphere. LCMS indicated the reaction was completed. THE was removed under reduced pressure. The residue was purified by reverse flash chromatography with the following conditions: column, C18 silica gel, 80 g, 20-35 um; mobile phase, water with 0.1% TFA and ACN (0% to 100% gradient in 50 min); detector, UV 254 nm. The collected fraction was lyophilized to afford 4-bromo-6-(1,2-dihydroxyethyl)-2,3-dihydroisoindol-1-one (6.7 g, 29%) as a brown solid. LCMS (ESI, ms): 272,274 [M+H]⁺.

### Step 13: Synthesis of Compound 15

To a stirred solution of 4-bromo-6-(1,2-dihydroxyethyl)-2,3-dihydroisoindol-1-one (5 g, 18.37 mmol, 1 equiv) in DCM (50 mL) was added imidazole (2.50 g, 36.75 mmol, 2 equiv) and TBDPSCl (5.05 g, 18.37 mmol, 1 equiv) dropwise at room temperature under air atmosphere. The resulting mixture was stirred for 2 h at room temperature under air atmosphere. LCMS indicated the reaction was completed. The resulting mixture was concentrated under reduced pressure. The residue was purified by silica gel column chromatography, eluted with PE/EA (1:1) to afford 4-bromo-6-{2-[(tert-butyldiphenylsilyl)oxy]-1-hydroxyethyl}-2,3-dihydroisoindol-1-one (3.6 g, 38%) as a white solid. LCMS:(ES·m/z):510,512[M+H]⁺.

### Step 14: Synthesis of Compound 16

To a stirred solution of 4-bromo-6-{2-[(tert-butyldiphenylsilyl)oxy]-1-hydroxyethyl}-2,3-dihydroisoindol-1-one (3.5 g, 6.85 mmol, 1 equiv) in DCM (70 mL) was added MsCl (0.94 g, 8.22 mmol, 1.2 equiv) and TEA (2.08 g, 20.56 mmol, 3 equiv) dropwise at room temperature under air atmosphere. The resulting mixture was stirred for 2 h at room temperature under air atmosphere. LCMS indicated the reaction was completed. The resulting mixture was concentrated under reduced pressure. The resulting mixture was used in the next step directly without further purification. LCMS:(ES·m/z):588,590[M+H]⁺.

### Step 15: Synthesis of Compound 17

To a stirred solution of 1-(7-bromo-3-oxo-1,2-dihydroisoindol-5-yl)-2-[(tert-butyldiphenylsilyl)oxy]ethyl methanesulfonate (3.5 g, 5.94 mmol, 1 equiv) and (3S)-3-methylpiperidine (0.71 g, 7.13 mmol, 1.2 equiv) in DMF (40 mL) was added K₂CO₃ (2.05 g, 14.86 mmol, 2.5 equiv) in portions at room temperature under air atmosphere. The resulting mixture was stirred for overnight at 50° C. under air atmosphere. LCMS indicated the reaction was completed. The mixture was allowed to cool down to room temperature. The reaction was quenched by the addition of Water/Ice (200 mL) at 0° C. The resulting mixture was extracted with EtOAc (3×100 mL). The combined organic layers were washed with brine (3×5 mL), dried over anhydrous Na₂SO₄. After filtration, the filtrate was concentrated under reduced pressure. The residue was purified by silica gel column chromatography, eluted with PE/EA (1:1) to afford 4-bromo-6-{2-[(tert-butyldiphenylsilyl)oxy]-1-[(3S)-3-methylpiperidin-1-yl]ethyl}-2,3-dihydroisoindol-1-one (2.8 g, 79%) as a yellow solid. LCMS:(ES·m/z):591,593[M+H]⁺.

### Step 16: Synthesis of Compound 18

To a stirred solution of 4-bromo-6-{2-[(tert-butyldiphenylsilyl)oxy]-1-[(3S)-3-methylpiperidin-1-yl]ethyl}-2,3-dihydroisoindol-1-one (2 g, 3.38 mmol, 1 equiv) in DMF (20 mL) was added (methylsulfanyl)sodium (0.36 g, 5.07 mmol, 1.5 equiv) in portions at room temperature under air atmosphere. The resulting mixture was stirred for 2 h at 100° C. LCMS indicated the reaction was completed. The mixture was allowed to cool down to room temperature. The residue was purified by reversed-phase flash chromatography with the following conditions: column, C18 silica gel; mobile phase, MeCN in Water (0.05% TFA), 0% to 50% gradient in 40 min; detector, UV 254 nm. The collected fraction was concentrated to afford 6-{2-[(tert-butyldiphenylsilyl)oxy]-1-[(3S)-3-methylpiperidin-1-yl]ethyl}-4-(methylsulfanyl)-2,3-dihydroisoindol-1-one (600 mg, 32%) as a yellow oil. LCMS:(ES·m/z):559[M+H]⁺.

### Step 17: Synthesis of Compound 20

To a stirred solution of 6-{2-[(tert-butyldiphenylsilyl)oxy]-1-[(3S)-3-methylpiperidin-1-yl]ethyl}-4-(methylsulfanyl)-2,3-dihydroisoindol-1-one (200 mg, 0.35 mmol, 1 equiv) in dioxane (4 mL) was added 6-chloro-N-ethyl-4-{3-[(4-methyl-1,2,4-triazol-3-yl)methyl]oxetan-3-yl}pyridin-2-amine (132 mg, 0.43 mmol, 1.2 equiv), Cs₂CO₃ (233 mg, 0.71 mmol, 2 equiv) and Xantphos (41 mg, 0.07 mmol, 0.2 equiv) and Pd(OAc)₂ (8 mg, 0.03 mmol, 0.1 equiv) in portions at room temperature under nitrogen atmosphere. The resulting mixture was stirred for 1.5 h at 110° C. under nitrogen atmosphere. The mixture was allowed to cool down to room temperature. LCMS indicated the reaction was ˜40% product. The resulting mixture was concentrated under reduced pressure. The residue was purified by silica gel column chromatography, eluted with CH₂Cl₂/MeOH (6:1) to afford 6-{2-[(tert-butyldiphenylsilyl)oxy]-1-[(3S)-3-methylpiperidin-1-yl]ethyl}-2-[6-(ethylamino)-4-{3-[(4-methyl-1,2,4-triazol-3-yl)methyl]oxetan-3-yl}pyridin-2-yl]-4-(methylsulfanyl)-3H-isoindol-1-one (70 mg, 27%) as a yellow solid. LCMS (ESI, m/z): 830 [M+H]⁺.

### Step 18: Synthesis of Compound 197

To a stirred solution of 6-{2-[(tert-butyldiphenylsilyl)oxy]-1-[(3S)-3-methylpiperidin-1-yl]ethyl}-2-[6-(ethylamino)-4-{3-[(4-methyl-1,2,4-triazol-3-yl)methyl]oxetan-3-yl}pyridin-2-yl]-4-(methylsulfanyl)-3H-isoindol-1-one (60 mg, 0.07 mmol, 1 equiv) in THE (1 mL) was added pyridine-hydrofluoride (36 mg, 0.36 mmol, 5 equiv) dropwise at 0° C. The resulting mixture was stirred for 1.5 h at room temperature. LCMS indicated the reaction was completed. The reaction mixture was concentrated to dryness under vacuum. The residue was purified by Prep-HPLC with the following conditions (Column: Xselect CSH Prep C18 C18 Column, 30*150 mm, 5 m; Mobile Phase A: Water (0.1% FA), Mobile Phase B: ACN; Flow rate: 60 mL/min mL/min; Gradient: 3% B to 33% B in 10 min; Wave Length: 254 nm/220 nm; RT1 (min): 8.68). The collected fraction was lyophilized to afford 2-[6-(ethylamino)-4-{3-[(4-methyl-1,2,4-triazol-3-yl)methyl]oxetan-3-yl}pyridin-2-yl]-6-{2-hydroxy-1-[(3S)-3-methylpiperidin-1-yl]ethyl}-4-(methylsulfanyl)-3H-isoindol-1-one (14.5 mg, 34%) as a white solid. LCMS (ESI, m/z): 592 [M+H]⁺. ¹H NMR (400 MHz, DMSO-d₆) δ 8.24 (s, 1H), 7.47 (s, 2H), 7.38 (s, 1H), 6.58-6.55 (t, J=6 Hz, 1H), 5.87 (s, 1H), 4.90 (d, J=4 Hz, 2H), 4.85 (s, 2H), 4.78-4.76 (m, 2H), 3.84-3.73 (m, 2H), 3.62-3.59 (m, 1H), 3.48 (s, 2H), 3.25-3.19 (m, 6H), 2.97-2.90 (m, 1H), 2.70-2.68 (m, 1H), 2.59 (s, 3H), 2.02-1.83 (m, 1H), 1.74-1.45 (m, 5H), 1.15 (t, J=6 Hz, 3H), 0.83-0.74 (m, 4H).

### Example 2: Binding Affinity of Example 1 Compounds to Cbl-b Protein

Compounds were assessed for binding activity to Cbl-b by their ability to compete with and displace a probe comprised of BODIPY-FL fluorophore conjugated to a Cbl-b inhibitor (see Example 54 in WO2020264398). In the assay, a 36-427 aa truncated form of Cbl-b with an N-terminus Avi-tag was incubated 20 mM HEPES pH 7.5, 150 mM NaCl, 0.01% Triton X-100, 0.1% BSA, 0.5 mM TCEP buffer with streptavidin-terbium (Cisbio). After one hour of incubation at room temperature, 1 uM each of fluorescent probe and compound were combined with the reaction mixture. Compounds with Cbl-b binding activity competitively displace the fluorophore tagged inhibitor thereby disrupting the FRET signal from the terbium-BODIPY-FL complex. The reaction mixtures were incubated for one additional hour at room temperature to allow for competition between inhibitor and candidate compounds to occur. The time resolved FRET (TR-FRET) signal was then read at 520/620 nM (excitation/emission) using a Spectramax M5e plate reader (Molecular Devices) to measure probe displacement. Compounds with Cbl-b binding activity had decreased FRET signal owing to disruption of the terbium-BODIPY-FL FRET complex. Standard methods in PRISM were used to calculate the compound IC50 values from the experimental data. Compounds were sorted by IC50 into ranges “A” through “D” as follows: “A” indicates a range of 1 nM-5 nM; “B” indicates a range of 5.01 nM-20 nM, “C” indicates a range of 20.01 nM-100 nM; and “D” indicates a range of 100.01 nM-1 μM.

TABLE 5
Binding Activity of Cbl-b Inhibitors

| Compound | Cb1-b | Compound | Cbl-b |
|---|---|---|---|
| Number | Activity (IC50) | Number | Activity (IC50) |
| 200 | B | 122 | A |
| 201 | A | 123 | A |
| 101 | B | 124 | D |
| 102 | B | 125 | B |
| 103 | B | 126 | A |
| 104 | D | 127 | C |
| 105 | D | 128 | C |
| 106 | D | 129 | C |
| 107 | B | 130 | B |
| 108 | A | 131 | C |
| 109 | D | 132 | C |
| 110 | A | 133 | B |
| 111 | B | 134 | B |
| 112 | C | 135 | B |
| 113 | C | 136 | B |
| 114A | D | 137 | B |
| 114B | D | 140 | B |
| 115 | D | 141A | B |
| 116 | B | 141B | B |
| 117 | C | 143 | A |
| 118 | A | 144 | B |
| 119 | A | 145 | B |
| 120 | C | 154 | A |
| 121 | C | 155 | A |
| 190 | B | 187 | C |
| 169 | B | 164 | B |
| 168 | C | 163 | B |
| 167 | B | 162 | B |
| 166 | B | 161 | A |
| 191 | D | 160 | A |
| 188 | D | 159 | C |
| 149 | C | 158 | A |
| 165 | B | 157 | C |
| 191 | B | 156 | C |
| 189 | B | 174 | B |
| 186 | B | 171 | D |
| 185 | B | 172 | D |
| 184 | B | 179 | D |
| 152 | A | 180 | D |
| 151 | D | 182 | B |
| 150 | A | 178 | D |
| 183 | D | 170 | A |
| 181 | A | 176 | B |
| 173 | A | 175 | A |
| 177 | A | | |

### Example 3: T Cell Activation by Cbl-b Inhibitors

Human primary T cells were isolated from commercially available frozen peripheral blood mononuclear cells (HemaCare Corporation) or from fresh LeukoPaks (Research Blood Components). T cells were isolated through depletion of non-T cells using negative selection kit according to the manufacturer's protocol (Miltenyi Biotec, Human Pan T Cell Isolation Kit, 130-096-535). Isolated cells were cultured overnight at 37° C. with 5% CO₂ and plated at 1×10⁵ cells per well in XVivo-15 serum-free media (Lonza, 04-418Q) into 96 well plates coated with anti-CD3 (OKT3) antibody (ThermoFisher, 16-0037-85). To coat the plates for T cell capture, anti-CD3 antibody was diluted in PBS at 10 μg/mL and 100 μl added to each well. Plates were incubated at 4° C. overnight or at 37° C. for 2 hours and washed once with PBS before use. Serial dilutions of each compound were added in duplicates to the T cells to assess their effect on T cell activation. T cells treated with 5 μg/mL of CD28 antibody (ThermoFisher, 16-0289-85) or T cells stimulated by CD3 only were used as a control. Plates were incubated with the Cbl-b inhibitors for 48 hours at 37° C. with 5% CO₂, and supernatants were subsequently analyzed for levels of the activation induced secreted cytokines IL-2 or IFN-y (BD Biosciences CBA Flex Beads, 558269, 558270). T cells were additionally stained with anti-CD69 antibody (Invitrogen, 12-0699-42 which labels a T cell activation associated membrane protein to and surface expression was measured by flow cytometry (ThermoFisher Attune NxT).

To control for natural inter-donor variability in T cell responses intrinsic to using primary human donor derived cells, secreted IL-2 and IFN-y measurements for each test compound were reported as the fold increase of the compound treated levels over background (antiCD3/CD28 treatment only) normalized to the values obtained for Compound 141A. Results for compound treatment-induced changes in CD69 levels are similarly reported as the fold increase due to compound treatment over anti-CD3 stimulated background only, normalized to values obtained for Compound 141A.

Normalized T cell IL-2 responses were sorted into ranges as follows: fold over background values between 1.41 and 1.8: range “X”, between 0.80 and 1.40: range “A”, values between 0.31 and 0.79: range “B” values between 0.10 and 0.30: range “C”, values between 0.01 and 0.09: range “D.”

Normalized T cell IFN-y responses were sorted into ranges as follows: fold over background between 1.10 and 2.00: range “A”, values between 0.31 and 1.00: range “B;” values between 0.10 and 0.30: range “C”, and values between 0.01 and 0.09: range “D.”

Similarly, bins for normalized CD69 levels were sorted into ranges as follows: fold over background values between 1.11 and 1.6: range “X”, values between 0.81 and 1.10: range “A”; values between 0.61 and 0.80: range “B;” values between 0.30 and 0.60: range “C”.

TABLE 6
T Cell Activity of Cbl-b Inhibitors

| Compound | IL-2 | | | IFN-y | | | CD69 | |
|---|---|---|---|---|---|---|---|---|
| Number | 100 nM | 10 nM | 1 nM | 100 nM | 10 nM | 1 nM | 100 nM | 10 nM |
| 148 | A | A | B | A | A | B | A | A |
| 209 | B | C | C | B | C | C | B | C |
| 118 | A | B | B | A | A | B | A | A |
| 130 | B | C | C | B | C | C | B | C |
| 100 | B | B | C | B | B | C | A | C |
| 126 | A | B | C | A | B | B | A | A |
| 141A | A | B | B | A | B | B | A | A |
| 141B | B | B | B | B | B | C | A | A |
| 124 | C | C | C | C | C | C | C | C |
| 117 | C | C | C | B | C | C | B | C |
| 116 | B | C | C | B | C | C | B | C |
| 123 | B | C | C | B | C | C | B | C |
| 115 | B | C | C | B | C | C | B | C |
| 121 | B | C | C | B | C | C | A | C |
| 125 | B | C | C | B | C | C | A | C |
| 134 | B | B | C | B | B | B | A | B |
| 135 | B | B | C | B | B | C | A | B |
| 136 | B | B | C | B | B | C | A | B |
| 137 | B | B | C | B | B | B | A | B |
| 122 | B | B | C | B | B | C | A | B |
| 120 | C | C | C | C | C | C | C | C |
| 119 | B | B | C | B | B | C | A | B |
| 113 | B | B | C | B | B | C | A | B |
| 112 | B | C | C | B | B | C | B | C |
| 111 | B | B | C | B | B | B | A | B |
| 200 | C | C | C | C | C | C | C | C |
| 201 | C | C | C | C | C | C | C | C |
| 102 | B | C | C | B | C | C | B | C |
| 103 | B | C | C | B | C | C | B | C |
| 107 | A | B | B | B | B | B | A | A |
| 110 | B | B | C | B | B | B | A | B |
| 106 | B | C | C | B | C | C | B | C |
| 147 | A | B | B | A | A | B | A | A |
| 146 | A | B | B | A | A | B | A | A |

TABLE 6A
T Cell Activity of Additional Cbl-b Inhibitors

| Compound | IL-2 | | | IFN-g | | | CD69 | | |
|---|---|---|---|---|---|---|---|---|---|
| Number | 100 nM | 10 nM | 1 nM | 100 nM | 10 nM | 1 nM | 100 nM | 10 nM | 1 nM |
| 190 | C | C | C | B | B | C | B | C | C |
| 169 | C | C | C | B | B | C | C | C | C |
| 164 | C | C | C | B | C | C | C | C | C |
| 162 | D | D | D | B | B | C | C | C | C |
| 154 | C | D | D | B | B | B | B | C | C |
| 186 | D | D | D | B | C | C | C | C | C |
| 152 | C | C | C | B | B | C | A | C | C |
| 150 | B | C | D | A | B | B | A | C | C |
| 181 | A | C | D | A | B | B | X | B | C |
| 173 | D | D | D | B | B | C | C | C | C |
| 174 | B | D | D | B | B | B | B | C | C |
| 171 | B | C | C | A | B | B | X | C | C |
| 182 | D | D | D | B | C | C | C | C | C |
| 170 | B | C | C | B | B | B | A | C | C |
| 176 | B | C | D | B | B | C | B | C | C |
| 175 | C | D | D | B | B | C | B | C | C |
| 192 | X | B | D | X | B | B | X | A | C |

### Example 4: Co-Crystallization of Representative Cbl-b Inhibitor and Cbl-b Protein

### Step 1: Production of Cbl-b Protein

Cbl-b (corresponding to 36-429th amino acids of Human Cbl-b gene, Uniprot Q13191) protein was produced as GST-fusion proteins as previously described (see Dou, H. et al., Nature Structural & Molecular Biology, 20 (8), 982-986). Briefly, Cbl-b protein was expressed in E. coli BL21(DE3), then purified by Glutathione-Sepharose affinity chromatography. GST-tag were cleaved from Cbl-b by the digestion of TEV protease, and further purified by S-sepharose cation exchange chromatography and Size-exclusion chromatography using superdex S-200. Protein was concentrated at 5 mg/ml in storage buffer (50 mM Tris-HCl, 150 mM NaCl, 1 mM DTT, 1000 Glycerol, pH 8.0).

### Step 2: X-Ray Co-crystallization and Data Collection

Purified Cbl-b (5.3 mg/ml, 116 μM) was mixed with 5-fold molar excess of the Cbl-b inhibitor, and crystallization was carried by hanging-drop diffusion methods in crystallization buffer containing 0.1M of Hepes (pH 7.0) or Tris (pH 8.0), 10-13% (w/v) of PEG 3350. Crystals were flash-frozen by liquid nitrogen in the presence of crystallization buffer supplemented with 25% (w/v) of glycerol as cryoprotectants. X-ray Diffraction data was collected at 11C beamline Pohang Accelerator Laboratory. Data was processed with XDS (see, Kabsch, W. XDS. Acta Cryst. D66, 125-132 (2010)) and structure was solved by molecular replacements using c-Cbl (PDB:2Y1M) (see, Dou, H.; et al. Nature Structural & Molecular Biology, 19(2), 184-192. doi:10.1038/nsmb.2231 (2012)) as search model. Molecular replacements and refinements were performed by Phenix (Liebschner, D., et al. Acta Crystallographica Section D: Structural Biology, 75(10), 861-877 (2019)).

### Step 3: Calculation of Ligand-Protein Interactions Analysis Between Cbl-b Inhibitors and Cbl-b Proteins

Comparison of complex structures of Cbl-b (36-427) with Compound 146, Compound 147, Compound 148, Compound 107, Compound 118, and Compound 122 revealed the key amino acid interactions between ligands and Cbl-b. There are several interactions conserved among the all chemicals such as pi-pi stacking interaction between triazole ring of ligand and Tyr363, carbonyl group in isoindoline ring interaction with backbone of Phe263, and protonated nitrogen in piperidine ring with Glu268.

To confirm the maintenance of these ligand-protein interactions, 10 ns of molecular dynamics (MD) simulations using dehydrated protein-ligand complex were carried out, and these interactions were maintained during simulations.

In the case of Compound 118 and Compound 122, which have longer substituent extension from central pyridine ring, distance between terminal nitrogen and His152 is in the range of 2.9-3.0 angstrom in crystal structure, while distance between terminal carbon of Compound 147 and His152 is 6.5 angstrom. The ligand:Hisl52 distances were maintained between 3-4 angstrom during 10 ns MD simulation in Compound 118 and Compound 122.

Differences in relative free energy between Compound 147 and Compound 118 were calculated by FEP+(Schrodinger)[6]. Addition of azide group in Compound 118 decreased free energy by 1.3 kcal/mol compared to Compound 147, indicating the displacement of additional water molecule and interaction with His152 may contribute to increased binding affinity for Compound 118 to Cbl-b.

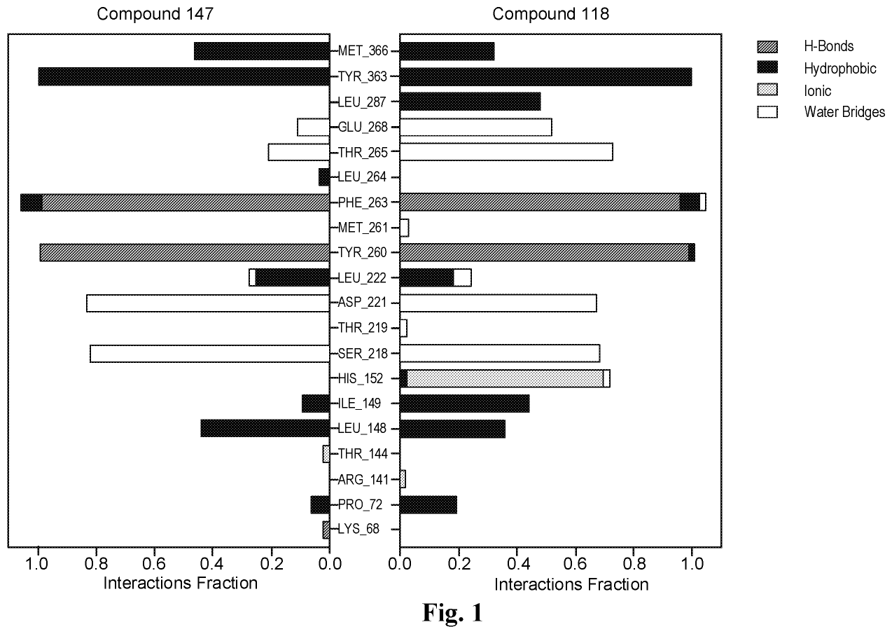

FIG. 1 is a histogram showing the protein-ligand interaction fractions between Cbl-b and Compound 118 and between Cbl-b and Compound 147. All residues that came into contact with the ligand throughout the MD trajectory are shown and are labeled by magnitude and interaction types. Compound 118 compared to Compound 147 (art compound). Compound 118 interacts with HIS152 residue of Cbl-b longer during the MD simulation than art compound Compound 147,

### Example 5: Procedure for Preparing Conjugate Precursors

### Synthesis of Compound 200

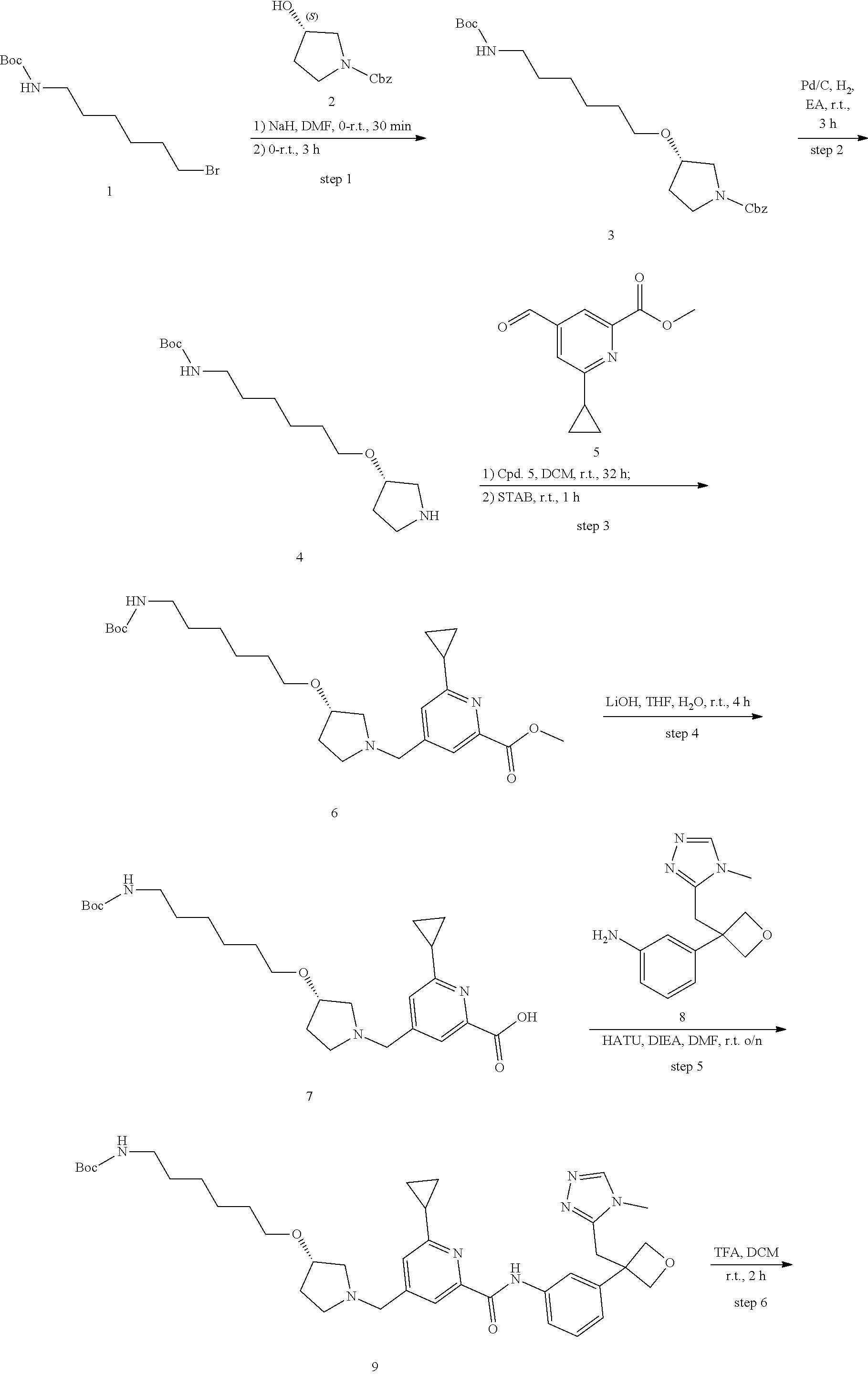

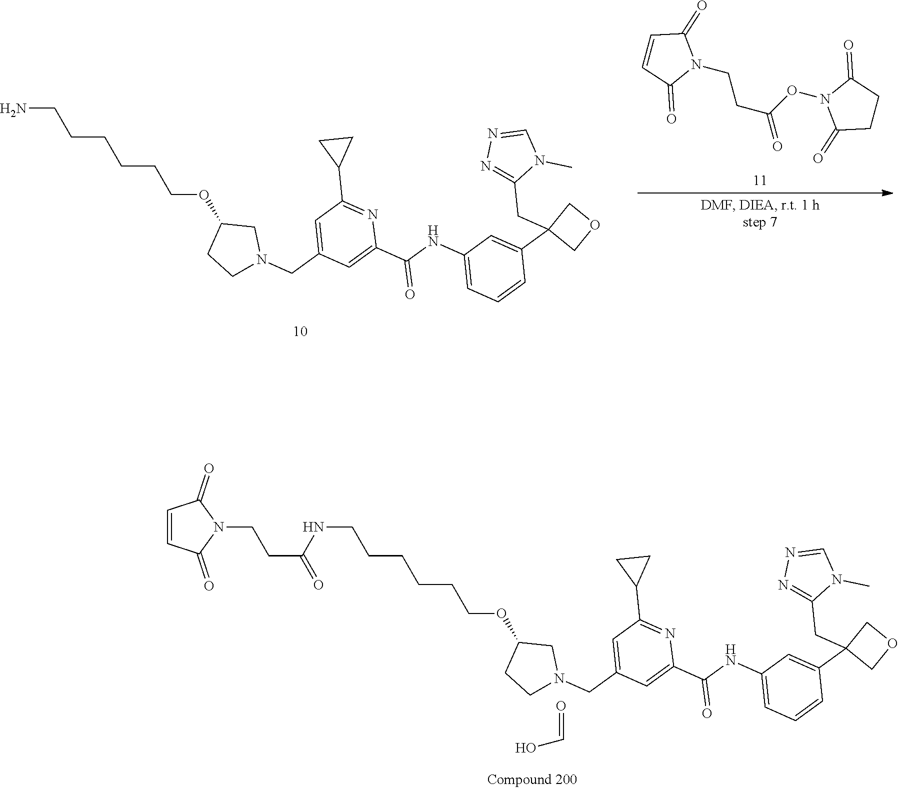

### Step 1: Synthesis of Compound 3

To a solution of benzyl (3S)-3-hydroxypyrrolidine-1-carboxylate (Compound 1, 0.79 g, 3.57 mmol, 1.00 equiv) in DMF (0.5 mL) at 0° C. was added NaH (0.17 g, 60%, 4.28 mmol, 1.2 equiv). The resulting mixture was stirred at room temperature for 0.5 h. Then tert-butyl N-(6-bromohexyl)carbamate (Compound 2, 1 g, 3.57 mmol, 1.00 equiv) was added at 0° C. The resulting mixture was stirred at room temperature for 3 hours. LCMS indicated the reaction was completed. The reaction was quenched with sat. aqueous NH₄Cl (50 mL). The resulting mixture was extracted with EA (25 mL×3). The combined organic layer was washed with brine (25 mL), dried over anhydrous sodium sulfate and concentrated to dryness under vacuum. The residue was purified by reverse phase column chromatography, eluted with (PE:EtOAc=1:2) to give benzyl (3S)-3-({6-[(tert-butoxycarbonyl)amino]hexyl}oxy)pyrrolidine-1-carboxylate (Compound 3, 650 mg, 36%) as a yellow oil. LCMS (ES, m/z): 421 [M+H]⁺

### Step 2: Synthesis of Compound 4

To a solution of benzyl (3S)-3-({6-[(tert-butoxycarbonyl)amino]hexyl}oxy)pyrrolidine-1-carboxylate (550 mg, 1.31 mmol, 1.00 equiv) in EtOAc (27 mL) was added Pd/C (139 mg, 10%) under N₂. Then the reaction mixture was stirred at room temperature for 3 hours under H2. LCMS indicated the reaction was completed. The reaction was filtered and the filtrate was concentrated to dryness under vacuum to give tert-butyl N-{6-[(3S)-pyrrolidin-3-yloxy]hexyl}carbamate (345 mg, 78%) as a yellow oil. LCMS (ES, m/z): 287 [M+H]⁺

### Step 3: Synthesis of Compound 6

A solution of tert-butyl N-{6-[(3S)-pyrrolidin-3-yloxy]hexyl}carbamate (Compound 4, 380 mg, 1.33 mmol, 1.00 equiv) and methyl 6-cyclopropyl-4-formylpyridine-2-carboxylate (Compound 5, prepared as described in WO2019048005, 272 mg, 1.33 mmol, 1.00 equiv) in DCM (19 mL) was stirred at room temperature for 2 hours, then STAB (562 mg, 2.65 mmol, 2.00 equiv) at room temperature. The resulting mixture was stirred at room temperature for 3 hours. LCMS indicated the reaction was completed. The reaction was quenched with methanol, concentrated to dryness under vacuum, The residue was purified by Prep-TLC (DCM:EtOAc=1:1) to give methyl 4-{[(3S)-3-({6-[(tert-butoxycarbonyl)amino]hexyl}oxy)pyrrolidin-1-yl]methyl}-6-cyclopropylpyridine-2-carboxylate (Compound 6, 370 mg, 55%) as a yellow oil. LCMS (ES, m/z): 476 [M+H]⁺

### Step 4: Synthesis of Compound 7

To a solution of methyl 4-{[(3S)-3-({6-[(tert-butoxycarbonyl)amino]hexyl}oxy)pyrrolidin-1-yl]methyl}-6-cyclopropylpyridine-2-carboxylate (Compound 6, 370 mg, 0.78 mmol, 1.00 equiv) in THE (5 mL)/water (1 mL) was added LiOH·H₂O (49 mg, 1.17 mmol, 1.5 equiv) at room temperature. The resulting mixture was stirred at room temperature overnight. LCMS indicated the reaction was completed. The resulting mixture was pacified to pH value to 3-4 with 1N HCl. The solvent was removed under vacuum. The residue was purified by reverse flash chromatography with the following conditions: column, C18 silica gel; mobile phase, ACN in water (0.1% FA), 0% to 60% gradient in 30 min; detector, UV 254 nm. The collected fraction was concentrated to dryness to give 4-{[(3S)-3-({6-[(tert-butoxycarbonyl)amino]hexyl}oxy)pyrrolidin-1-yl]methyl}-6-cyclopropylpyridine-2-carboxylic acid (Compound 7, 320 mg, 74%) as a yellow solid. LCMS (ES, m/z): 462 [M+H]⁺

### Step 5: Synthesis of Compound 9

A solution of 4-{[(3S)-3-({6-[(tert-butoxycarbonyl)amino]hexyl}oxy)pyrrolidin-1-yl]methyl}-6-cyclopropylpyridine-2-carboxylic acid (Compound 7, 310 mg, 0.67 mmol, 1.00 equiv) and HATU (383 mg, 1.01 mmol, 1.5 equiv) in DMF (16 mL) was stirred at room temperature for 0.5 hour. Then 3-{3-[(4-methyl-1,2,4-triazol-3-yl)methyl]oxetan-3-yl}aniline (Compound 8, prepared as described in WO20201021761, 197 mg, 0.81 mmol, 1.20 equiv) and DIEA (260 mg, 2.02 mmol, 3.00 equiv) was added at room temperature. The resulting mixture was stirred at room temperature overnight. LCMS indicated the reaction was completed. The resulting mixture was diluted with water (150 mL), extracted with EtOAc (50 mL*3), the combined organic layer was washed with brine (50 mL), dried over anhydrous sodium sulfate and concentrated to dryness under vacuum. The residue was purified with Prep-TLC (DCM:MeOH=10:1) to give tert-butyl N-(6-{[(3S)-1-({2-cyclopropyl-6-[(3-{3-[(4-methyl-1,2,4-triazol-3-yl)methyl]oxetan-3-yl}phenyl)carbamoyl]pyridin-4-yl}methyl)pyrrolidin-3-yl]oxy}hexyl)carbamate (Compound 9, 280 mg, 54%) as a yellow solid. LCMS (ES, m/z): 688 [M+H]⁺

### Step 6: Synthesis of Compound 10

To a solution of tert-butyl N-(6-{[(3S)-1-({2-cyclopropyl-6-[(3-{3-[(4-methyl-1,2,4-triazol-3-yl)methyl]oxetan-3-yl}phenyl)carbamoyl]pyridin-4-yl}methyl)pyrrolidin-3-yl]oxy}hexyl)carbamate (Compound 9, 270 mg, 0.39 mmol, 1.00 equiv) in DCM (10 mL) was added TFA (1 mL) at room temperature. The resulting mixture was stirred at room temperature for 5 hours. LCMS indicated the reaction was completed. The resulting mixture was added into a.q. NaHCO₃ (100 mL). The organic layer was separated out. The water phase was extracted with DCM (30 mL*2). The combined organic layer was washed with water (30 mL), brine (30 mL), dried over anhydrous sodium sulfate to give 4-{[(3S)-3-[(6-aminohexyl)oxy]pyrrolidin-1-yl]methyl}-6-cyclopropyl-N-(3-{3-[(4-methyl-1,2,4-triazol-3-yl)methyl]oxetan-3-yl}phenyl)pyridine-2-carboxamide (210 mg, 85%) as a yellow solid. LCMS (ES, m/z): 588 [M+H]⁺

### Step 7: Synthesis of Compound 200

A solution of 4-{[(3S)-3-[(6-aminohexyl)oxy]pyrrolidin-1-yl]methyl}-6-cyclopropyl-N-(3-{3-[(4-methyl-1,2,4-triazol-3-yl)methyl]oxetan-3-yl}phenyl)pyridine-2-carboxamide (Compound 10, 100 mg, 0.17 mmol, 1.00 equiv), 2,5-dioxopyrrolidin-1-yl 3-(2,5-dioxopyrrol-1-yl)propanoate (Compound 11, 45 mg, 0.17 mmol, 1.00 equiv) and DIEA (44 mg, 0.34 mmol, 2.00 equiv) in DMF (0.5 mL) was stirred at room temperature for 1 h. LCMS indicated the reaction was completed. After filtration, the filtrate was purified by reverse flash chromatography with the following conditions: XBridge Shield RP18 OBD Column, 19×150 mm, 5 m; Mobile Phase A: Water (0.05% FA), Mobile Phase B: ACN; Flow rate: 25 mL/min; Gradient: 13% B to 43% B in 7 min, 43% B; Wave Length: 254 nm. The collected fraction was lyophilized to give (37.7 mg, 28%) as a white solid. LCMS (ES, m/z): 739 [M+H]⁺; ¹H NMR (400 MHz, DMSO-d₆) δ10.13 (s, 1H), 8.20 (s, 1H), 8.14 (s, 1H), 7.90-7.88 (m, 1H), 7.83 (s, 1H), 7.74 (d, J=9.2 Hz, 1H), 7.43-7.41 (m, 2H), 7.28 (t, J=7.6 Hz, 1H), 7.00 (s, 2H), 6.66 (d, J=8.0 Hz, 1H), 4.95-4.86 (m, 4H), 4.00 (br, 1H), 3.71 (s, 2H), 3.59 (t, J=7.2 Hz, 2H), 3.49 (s, 2H), 3.34-3.26 (m, 4H), 2.99-2.76 (m, 5H), 2.78-2.76 (m, 1H), 2.65-2.60 (m, 1H), 2.30 (t, J=7.6 Hz, 2H), 2.26-2.22 (m, 1H), 2.04-2.03 (m, 1H), 1.78-1.72 (m, 1H), 1.47-1.43 (m, 2H), 1.35-1.28 (m, 2H), 1.28-1.21 (m, 4H), 1.17-1.13 (m, 2H), 1.06-1.01 (m, 2H).

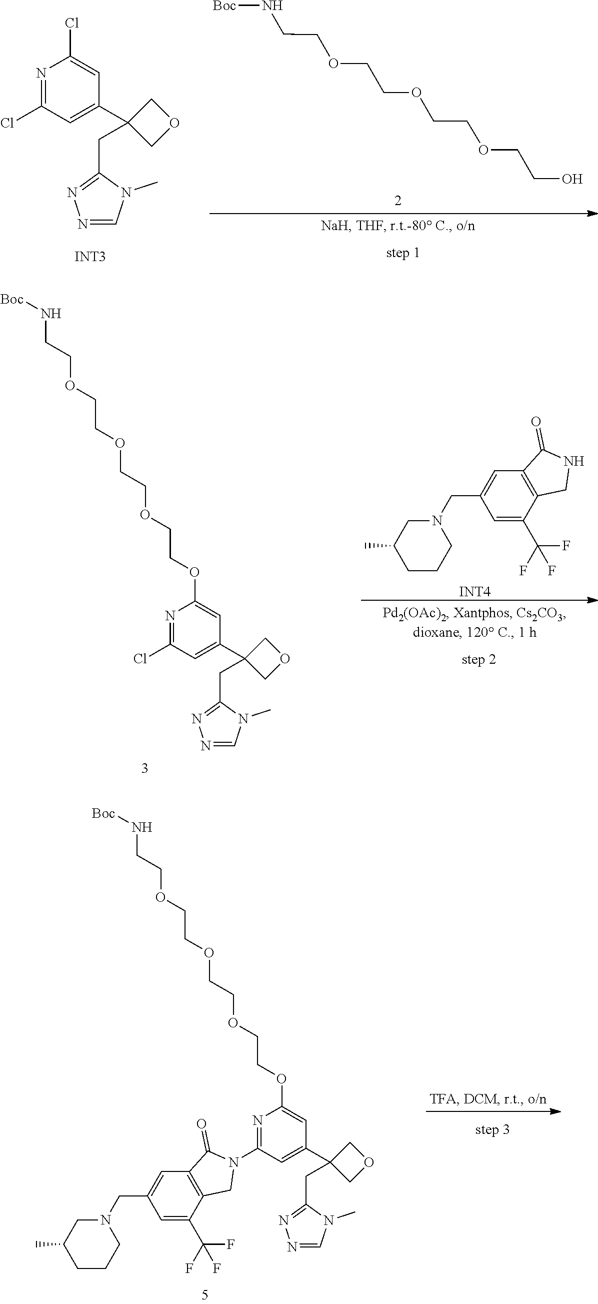

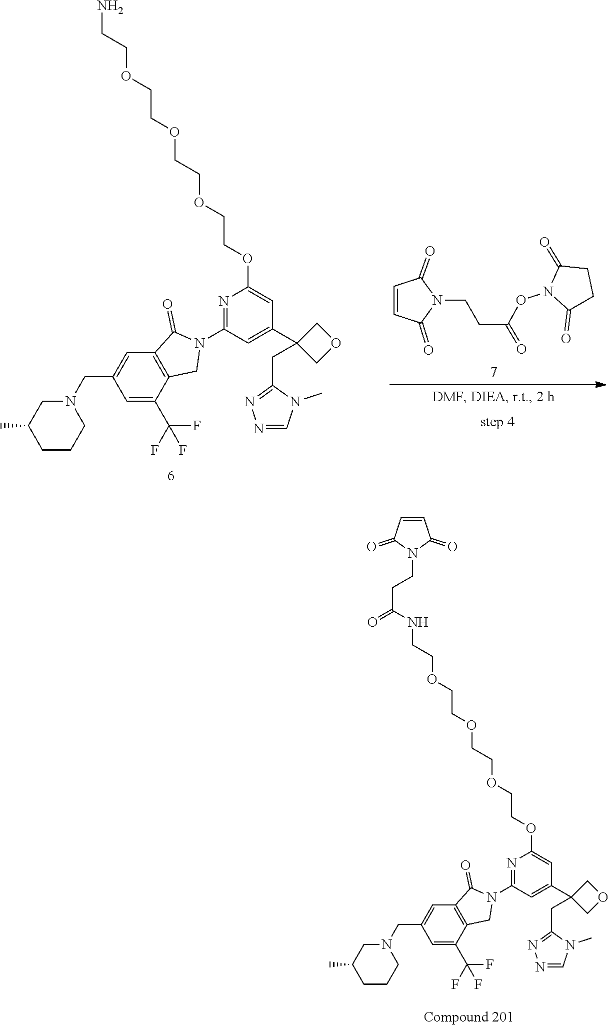 [SMILES: C[C@H]1CCCN(Cc2cc3c(c(C(F)(F)F)c2)CN(c2cc(C4(Cc5nncn5C)COC4)cc(OCCOCCOCCOCCN)n2)C3=O)C1 ; O=C(CCN1C(=O)C=CC1=O)ON1C(=O)CCC1=O ; C[C@H]1CCCN(Cc2cc3c(c(C(F)(F)F)c2)CN(c2cc(C4(Cc5nncn5C)COC4)cc(OCCOCCOCCOCCNC(=O)CCN4C(=O)C=CC4=O)n2)C3=O)C1]

### Step 1: Synthesis of Compound 3

To sealed tube solution of tert-butyl N-(2-{2-[2-(2-hydroxyethoxy)ethoxy]ethoxy}ethyl)carbamate (Compound 2, 294 mg, 1.00 mmol, 1.2 equiv) in THE (5 mL) was added NaH (50 mg, 60% 1.25 mmol, 1.5 equiv) at room temperature under N₂. The reaction was stirred at room temperature for 0.5 h. Then 2,6-dichloro-4-{3-[(4-methyl-1,2,4-triazol-3-yl)methyl]oxetan-3-yl}pyridine (INT3, 250 mg, 0.84 mmol, 1.00 equiv) was added at room temperature under N₂. The resulting mixture was stirred at 80° C. overnight. LCMS indicated the reaction was completed. After cooled to room temperature, the reaction was filtered, the filtrate was purified by reverse flash chromatography with the following conditions: column, C18 silica gel; mobile phase, ACN in water (0.1% FA), 0% to 60% gradient in 30 min; detector, UV 254 nm. The collected fraction was concentrated to give tert-butyl N-{2-[2-(2-{2-[(6-chloro-4-{3-[(4-methyl-1,2,4-triazol-3-yl)methyl]oxetan-3-yl}pyridin-2-yl)oxy]ethoxy}ethoxy)ethoxy]ethyl}carbamate (Compound 3, 270 mg, 55%) as a colorless oil. LCMS (ES, m/z): 556,558 [M+H]⁺

### Step 2: Synthesis of Compound 5

To a solution of tert-butyl N-{2-[2-(2-{2-[(6-chloro-4-{3-[(4-methyl-1,2,4-triazol-3-yl)methyl]oxetan-3-yl}pyridin-2-yl)oxy]ethoxy}ethoxy)ethoxy]ethyl}carbamate (Compound 3, 150 mg, 0.27 mmol, 1.00 equiv) and 6-{[(3S)-3-methylpiperidin-1-yl]methyl}-4-(trifluoromethyl)-2,3-dihydroisoindol-1-one (INT4, 84 mg, 0.27 mmol, 1.00 equiv) in dioxane (8 mL) were added Cs₂CO₃ (264 mg, 0.81 mmol, 3.00 equiv), XantPhos (31 mg, 0.05 mmol, 0.2 equiv) and Pd(OAc)₂ (6 mg, 0.03 mmol, 0.10 equiv) under N₂. The resulting mixture was stirred at 120° C. for 1 under N₂. LCMS indicated the reaction was completed. After cooled to room temperature, the reaction was filtered, the filtrate was purified with Prep-TLC (DCM:MeOH=10:1) to give tert-butyl N-{2-[2-(2-{2-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)oxy]ethoxy}ethoxy)ethoxy]ethyl}carbamate (Compound 5, 150 mg, 62%) as a yellow solid. LCMS (ES, m/z): 832 [M+H]⁺

### Step 3: Synthesis of Compound 6

A solution of tert-butyl N-{2-[2-(2-{2-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)oxy]ethoxy}ethoxy)ethoxy]ethyl}carbamate (Compound 5, 90 mg, 0.11 mmol, 1.00 equiv) in TFA (450 uL)/DCM (4.5 mL) was stirred at room temperature overnight. LCMS indicated the reaction was completed. The reaction was concentrated to dryness under vacuum. The residue was purified by reverse flash chromatography with the following conditions: column, C18 silica gel; mobile phase, ACN in water (0.1% FA), 0% to 60% gradient in 30 min; detector, UV 254 nm&220 nm. The collected fraction was concentrated to give 2-[6-(2-{2-[2-(2-aminoethoxy)ethoxy]ethoxy}ethoxy)-4-{3-[(4-methyl-1,2,4-triazol-3-yl)methyl]oxetan-3-yl}pyridin-2-yl]-6-{[(3S)-3-methylpiperidin-1-yl]methyl}-4-(trifluoromethyl)-3H-isoindol-1-one (Compound 6, 85 mg, 96%) as a yellow oil. MS: (ES, m/s): 732 [M+H]⁺

### Step 4: Synthesis of Compound 201

To a solution of 2-[6-(2-{2-[2-(2-aminoethoxy)ethoxy]ethoxy}ethoxy)-4-{3-[(4-methyl-1,2,4-triazol-3-yl)methyl]oxetan-3-yl}pyridin-2-yl]-6-{[(3S)-3-methylpiperidin-1-yl]methyl}-4-(trifluoromethyl)-3H-isoindol-1-one (Compound 6, 40 mg, 0.06 mmol, 1.00 equiv) in DMF (2 mL) were added DIEA (21 mg, 0.17 mmol, 3.00 equiv) and 2,5-dioxopyrrolidin-1-yl 3-(2,5-dioxopyrrol-1-yl)propanoate (1.82 mg, 0.01 mmol, 1.00 equiv) at room temperature. The resulting mixture was stirred at room temperature for 2 hours. LCMS indicated the reaction was completed. The reaction mixture was purified by reverse flash chromatography with the following conditions: Column: XSelect CSH Prep C18 OBD Column, 19×250 mm, 5 m; Mobile Phase A: Water (0.05% FA), Mobile Phase B: ACN; Flow rate: 25 mL/min; Gradient: 14% B to 44% B in 7 min, 44% B; Wave Length: 254 nm; The collected fraction was lyophilized to give 3-(2,5-dioxopyrrol-1-yl)-N-{2-[2-(2-{2-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)oxy]ethoxy}ethoxy)ethoxy]ethyl}propanamide (Compound 201, 14.6 mg, 28%) as a white solid. MS: (ES, m/s): 883 [M+H]⁺; ¹H NMR (400 MHz, DMSO-d₆) δ8.25 (s, 1H), 8.03-7.94 (m, 4H), 7.00 (s, 2H), 6.48 (s, 1H), 5.21 (s, 2H), 4.92-4.82 (m, 4H), 4.40-4.38 (m, 2H), 3.79-3.76 (m, 2H), 3.65 (s, 2H), 3.60-3.47 (m, 12H), 3.36-3.35 (m, 5H), 3.15-3.11 (m, 2H), 2.74-2.67 (m, 2H), 2.31 (t, J=7.6 Hz, 2H), 1.96-1.91 (m, 1H), 1.68-1.59 (m, 4H), 1.52-1.46 (m, 1H), 0.88-0.81 (m, 4H).

### Synthesis of Compound 202

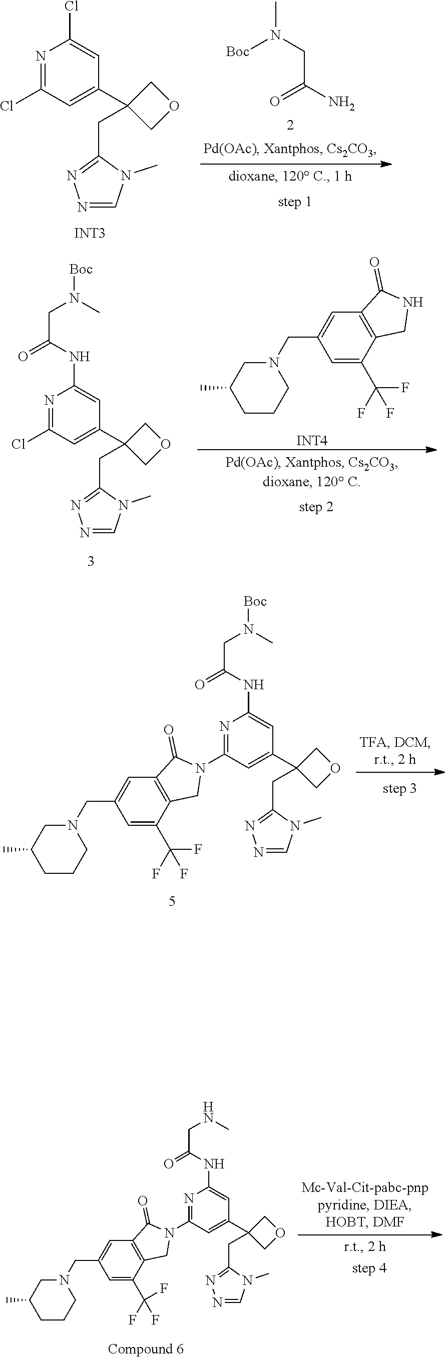

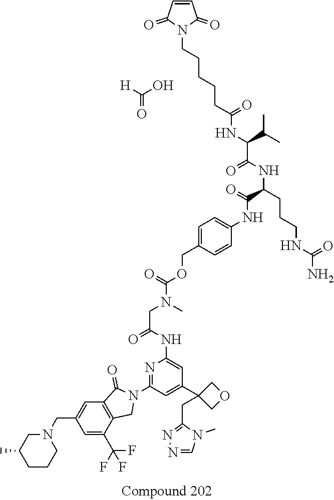 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CC(=O)Nc2cc(C3(Cc4nncn4C)COC3)cc(N3Cc4c(cc(CN5CCC[C@H](C)C5)cc4C(F)(F)F)C3=O)n2)cc1 ; O=CO]

### Step 1: Synthesis of Compound 3

A solution of 2,6-dichloro-4-{3-[(4-methyl-1,2,4-triazol-3-yl)methyl]oxetan-3-yl}pyridine (INT3, 400 mg, 1.34 mmol, 1.00 equiv), tert-butyl N-(carbamoylmethyl)-N-methylcarbamate (Compound 2, 252 mg, 1.34 mmol, 1.00 equiv), XantPhos (155 mg, 0.27 mmol, 0.20 equiv), Cs₂CO₃ (1307 mg, 4.01 mmol, 3.00 equiv) and Pd(OAc)₂ (30 mg, 0.13 mmol, 0.10 equiv) in dioxane (6 mL) was stirred at 120° C. for 1 h. LCMS indicated the reaction was completed. After cooled to room temperature. The reaction was filtered. The filtrate was concentrated to dryness under vacuum. The residue was purified by Prep-TLC (DCM:MeOH=10:1) to give tert-butyl N-{[(6-chloro-4-{3-[(4-methyl-1,2,4-triazol-3-yl)methyl]oxetan-3-yl}pyridin-2-yl)carbamoyl]methyl}-N-methylcarbamate (Compound 3, 390 mg, 63%) as a green solid. LCMS (ES, m/z): 451,453 [M+H]⁺

### Step 2: Synthesis of Compound 5

A solution of tert-butyl N-{[(6-chloro-4-{3-[(4-methyl-1,2,4-triazol-3-yl)methyl]oxetan-3-yl}pyridin-2-yl)carbamoyl]methyl}-N-methylcarbamate (Compound 3, 280 mg, 0.62 mmol, 1.00 equiv), 6-{[(3S)-3-methylpiperidin-1-yl]methyl}-4-(trifluoromethyl)-2,3-dihydroisoindol-1-one (INT4, 194 mg, 0.62 mmol, 1.00 equiv), XantPhos (72 mg, 0.12 mmol, 0.20 equiv), Cs₂CO₃ (607 mg, 1.86 mmol, 3.00 equiv) and Pd(OAc)₂ (14 mg, 0.06 mmol, 0.10 equiv) in dioxane (6 mL) was stirred at 120° C. for 2 hours. LCMS indicated the reaction was completed. After cooled to room temperature. The reaction was filtered and concentrated to dryness under vacuum. The residue was purified by Prep-TLC (DCM:MeOH=10:1) to give tert-butyl N-methyl-N-{[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamoyl]methyl}carbamate (Compound 5, 200 mg, 39%) as a yellow solid. LCMS (ES, m/z): 727 [M+H]⁺

### Step 3: Synthesis of Compound 6

Tert-butyl N-methyl-N-{[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamoyl]methyl}carbamate (Compound 5, 250 mg, 0.34 mmol, 1.00 equiv) in TFA (400 uL) and DCM (4 mL) was stirred at room temperature for 2 hours. LCMS indicated the reaction was completed. The reaction was concentrated to dryness under vacuum. The residue was purified by reverse flash chromatography with the following conditions: column, C18 silica gel; mobile phase, ACN in water (0.1% FA), 0% to 60% gradient in 30 min; detector, UV 254 nm. The collected fraction was concentrated to give N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)-2-(methylamino)acetamide; formic acid (Compound 6, 100 mg, 41%) as a white solid. LCMS (ES, m/z): 627 [M+H]⁺; ¹H NMR (400 MHz, DMSO-d₆) δ8.24 (s, 1H), 8.20 (s, 2H), 8.08 (s, 1H), 7.99 (s, 1H), 7.95 (s, 1H), 7.75 (br, 1H), 5.19 (s, 2H), 4.98-4.81 (m, 4H), 3.65 (s, 2H), 3.59 (s, 2H), 3.52-3.50 (m, 2H), 3.33 (s, 3H), 2.74-2.67 (m, 2H), 2.42 (s, 3H), 1.97-1.92 (m, 1H), 1.68-1.59 (m, 4H), 1.52-1.46 (m, 1H), 0.88-0.81 (m, 4H).

### Step 4: Synthesis of Compound 202

To a solution of N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)-2-(methylamino)acetamide (Compound 6, 65 mg, 0.10 mmol, 1.00 equiv) in DMF (2 mL) were added HOBT (84 mg, 0.62 mmol, 6.00 equiv), pyridine (41 mg, 0.52 mmol, 5.00 equiv), DIEA (67 mg, 0.52 mmol, 5.00 equiv) and {4-[(2S)-5-(carbamoylamino)-2-[(2S)-2-[6-(2,5-dioxopyrrol-1-yl)hexanamido]-3-methylbutanamido]pentanamido]phenyl}methyl 4-nitrophenyl carbonate (77 mg, 0.10 mmol, 1.00 equiv) at room temperature. The resulting mixture was stirred at room temperature for 2 hours. LCMS indicated the reaction was completed. The resulting mixture was purified by Column: XSelect CSH Prep C18 OBD Column, 19*250 mm, 5 m; Mobile Phase A: Water (0.1% FA), Mobile Phase B: ACN; Flow rate: 25 mL/min; Gradient: 15% B to 35% B in 10 min, 35% B; Wave Length: 220 nm; The collected fraction was lyophilized to give formic acid; {4-[(2S)-5-(carbamoylamino)-2-[(2S)-2-[6-(2,5-dioxopyrrol-1-yl)hexanamido]-3-methylbutanamido]pentanamido]phenyl}methyl N-methyl-N-{[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamoyl]methyl}carbamate (Compound 202, 22 mg, 15%) as a yellow solid. LCMS (ES, m/z): 1226 [M+H]f; ¹H NMR (400 MHz, DMSO-d₆) δ 8.47-8.44 (m, 1H), 8.29-8.21 (m, 2H), 8.19-8.15 (m, 1H), 8.13-8.04 (m, 2H), 8.00-7.92 (m, 2H), 7.89-7.72 (m, 2H), 7.68-7.66 (m, 1H), 7.50-7.41 (m, 4H), 7.35-7.23 (m, 4H), 7.20-7.11 (m, 1H), 6.99 (s, 2H), 5.25-5.12 (m, 2H), 5.08-5.00 (m, 1H), 4.72 (d, J=6 Hz, 2H), 4.53-4.50 (m, 2H), 3.76-3.69 (m, 5H), 3.68-3.65 (m, 4H), 3.36 (t, J=7.2 Hz, 2H), 3.24-3.20 (m, 2H), 3.10-3.03 (m, 4H), 2.82-2.78 (m, 2H), 2.60 (s, 3H), 2.41 (s, 3H), 2.23-2.19 (m, 2H), 2.19-2.01 (m, 3H), 1.62 (s, 3H), 1.59-1.55 (m, 2H), 1.50-1.42 (m, 6H), 1.33-1.31 (m, 2H), 1.20-1.16 (m, 2H).

### Synthesis of Compound 203

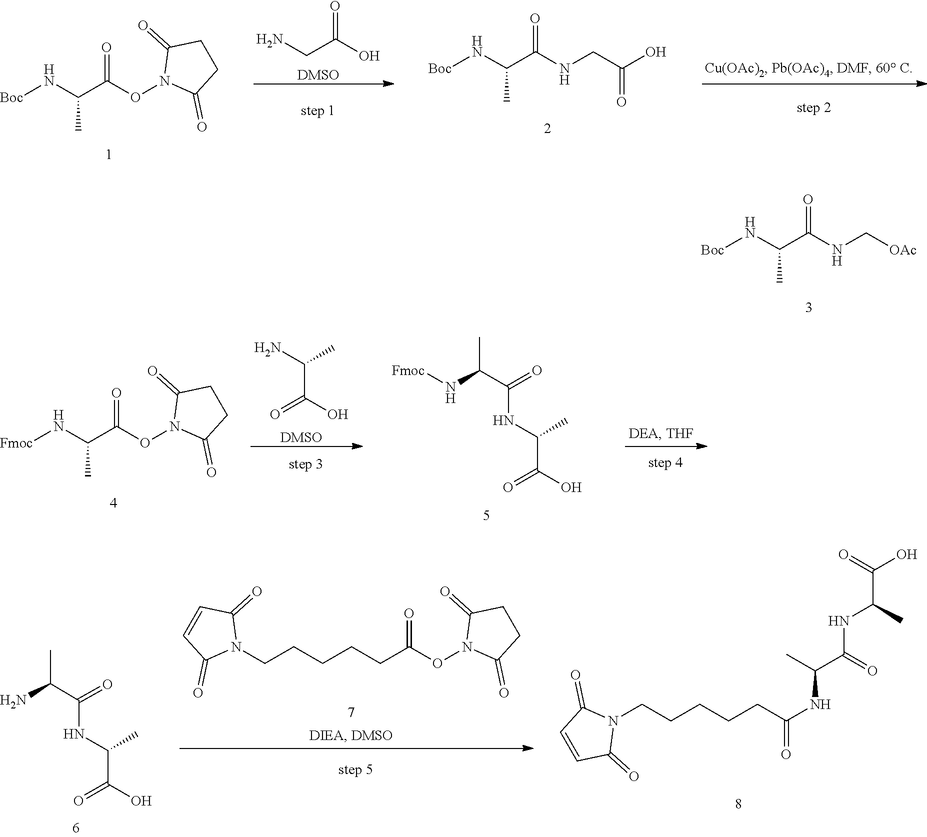

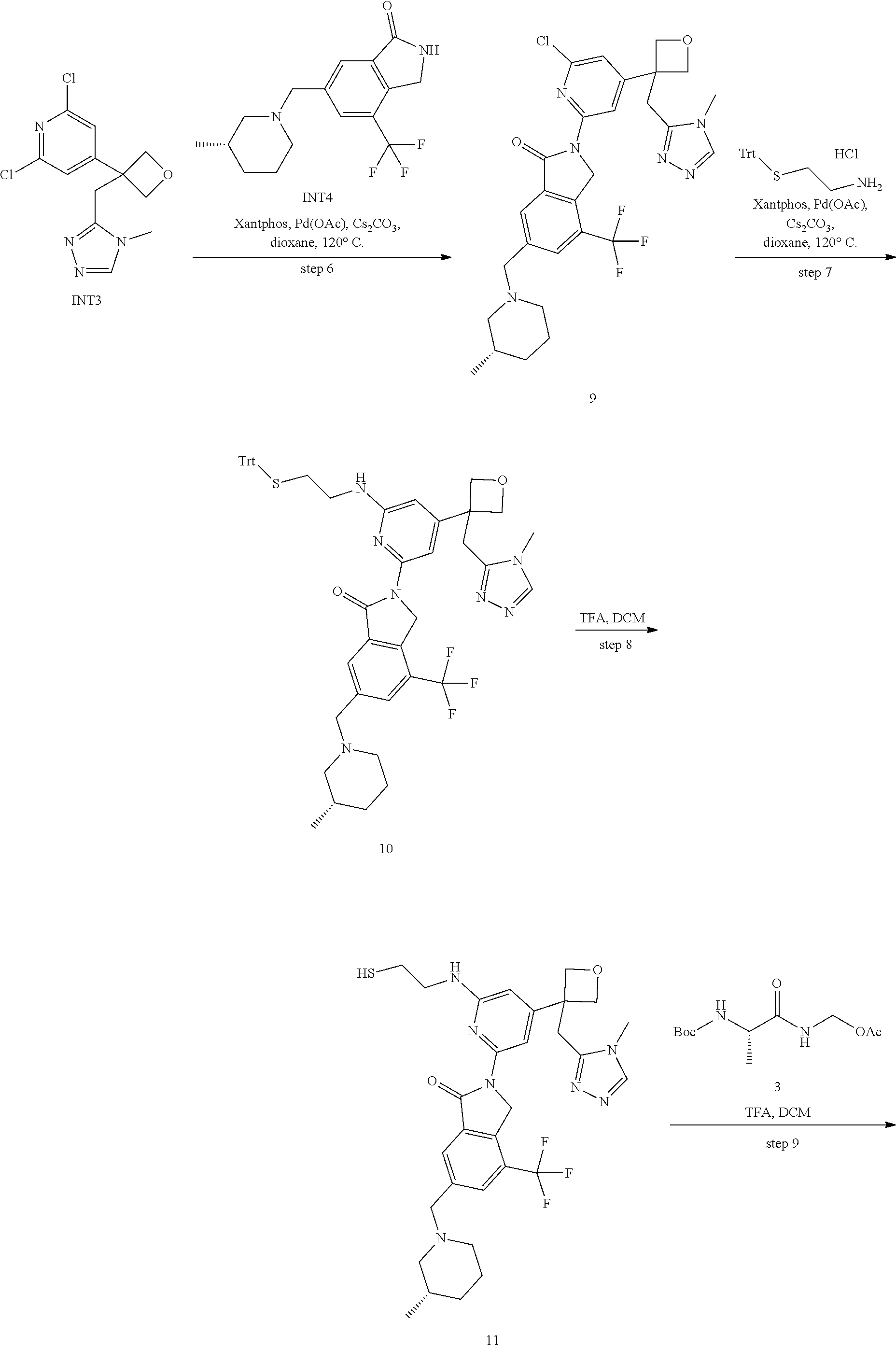

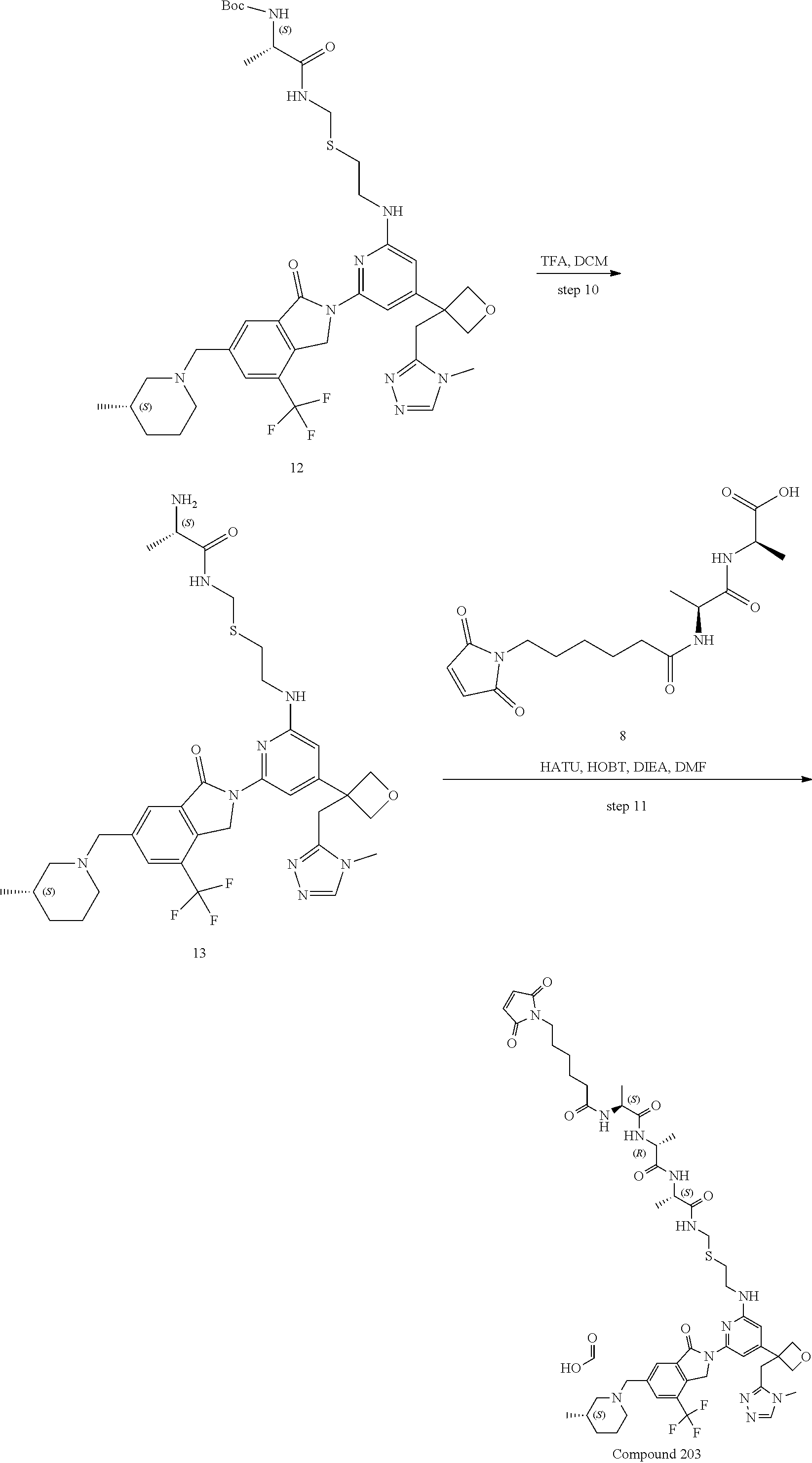

### Step 1: Synthesis of Compound 2

To a stirred solution of 2,5-dioxopyrrolidin-1-yl (2S)-2-[(tert-butoxycarbonyl)amino]propanoate (Compound 1, 5.0 g, 17.46 mmol, 1.0 equiv) in DMSO (50 mL) was added glycine (1.57 g, 20.95 mmol, 1.2 equiv) in portions at room temperature under nitrogen atmosphere. The resulting mixture was stirred for overnight at room temperature under nitrogen atmosphere. LCMS indicated the reaction was completed. The reaction mixture was purified by reverse flash chromatography with the following conditions: column, C18 silica gel; mobile phase, ACN in water (0.1% FA), 10% to 30% gradient in 40 min; detector, UV 224 nm and 200 nm. The resulting mixture was concentrated under vacuum to afford [(2S)-2-[(tert-butoxycarbonyl)amino]propanamido]acetic acid (Compound 2, 1.2 g, 25%) as a light yellow semi-solid. LCMS: (ES, m/z): 247 [M+H]⁺, 147 [M+H−100]⁺.

### Step 2: Synthesis of Compound 3

To a stirred solution of [(2S)-2-[(tert-butoxycarbonyl)amino]propanamido]acetic acid (Compound 2, 1.1 g, 4.46 mmol, 1.0 equiv) in DMF (11 mL) were added Cu(OAc)₂ (0.16 g, 0.89 mmol, 0.2 equiv) and Pb(OAc)₄ (5.94 g, 13.40 mmol, 3.0 equiv) in portions at room temperature under nitrogen atmosphere. The resulting mixture was stirred for 6 h at 60° C. under nitrogen atmosphere. LCMS indicated the reaction was completed. The reaction mixture was cooled down to room temperature. The reaction mixture was purified by reverse flash chromatography with the following conditions: column, C18 silica gel; mobile phase, ACN in water (0.1% FA), 10% to 30% gradient in 30 min; detector, UV 200 nm and 220 nm. The resulting mixture was lyophilized to afford [(2S)-2-[(tert-butoxycarbonyl)amino]propanamido]methyl acetate (Compound 3, 700 mg, 60%) as a white solid. LCMS: (ES, m/z): 283 [M+Na]⁺, 261 [M+H]⁺.

### Step 3: Synthesis of Compound 5

To a stirred solution of 2,5-dioxopyrrolidin-1-yl (2S)-2-{[(9H-fluoren-9-ylmethoxy)carbonyl]amino}propanoate (25 g, 61.21 mmol, 1 equiv) in DMSO (250 mL) was added D-alanine (6.54 g, 73.45 mmol, 1.2 equiv) at room temperature under nitrogen atmosphere. The resulting mixture was stirred for overnight at room temperature under nitrogen atmosphere. LCMS indicated the reaction was completed. The reaction was quenched with water/ice at 0° C. The precipitated solids were collected by filtration and washed with Et₂O (3×50 mL). The precipitated solid was purified by silica gel column chromatography, eluted with PE/EA (1:1) to afford (2R)-2-[(2S)-2-{[(9H-fluoren-9-ylmethoxy)carbonyl]amino}propanamido]propanoic acid (12.05 g, 51%) as a white solid. LCMS (ESI, m/z): 383[M+H]⁺

### Step 4: Synthesis of Compound 6

To a stirred solution of (2R)-2-[(2S)-2-{[(9H-fluoren-9-ylmethoxy)carbonyl]amino}propanamido]propanoic acid (Compound 5, 6 g, 15.69 mmol, 1 equiv) in THE (120 mL) was added DEA (12 mL) dropwise at room temperature. The resulting mixture was stirred for overnight at room temperature under nitrogen atmosphere. LCMS indicated the reaction was completed. The resulting mixture was concentrated under vacuum. The residue was dissolved in water (50 mL). The resulting mixture was extracted with EtOAc (3×20 mL). The aqueous layers was concentrated under reduced pressure. The residue was purified by trituration with MeCN (5 mL). The precipitated solids were collected by filtration and washed with MeCN (3×5 mL). This resulted in D-alanyl-D-alanine (Compound 6, 1.2 g, 47%) as a white solid. LCMS (ESI, m/z):161[M+H]⁺

### Step 5: Synthesis of Compound 8

To a stirred solution of D-alanyl-D-alanine (Compound 6, 1.2 g, 7.49 mmol, 1 equiv) in DMSO (24 mL) was added 2,5-dioxopyrrolidin-1-yl 6-(2,5-dioxopyrrol-1-yl)hexanoate (Compound 7, 2.77 g, 8.99 mmol, 1.2 equiv) in portions at room temperature. The resulting mixture was stirred for overnight at room temperature under nitrogen atmosphere. LCMS indicated the reaction was completed. The reaction mixture was purified by reverse flash chromatography with the following conditions: column, C18 silica gel; mobile phase, MeCN in water (0.05% TFA), 5% to 50% gradient in 30 min; detector, UV 254 nm. The collected fraction was concentrated to afford (2R)-2-[(2S)-2-[6-(2,5-dioxopyrrol-1-yl)hexanamido]propanamido]propanoic acid (Compound 8, 1.4 g, 52%) as a white solid. LCMS (ESI, m/z):354[M+H]⁺

### Step 6: Synthesis of Compound 9

The mixture of 2,6-dichloro-4-{3-[(4-methyl-1,2,4-triazol-3-yl)methyl]oxetan-3-yl}pyridine (INT3, 1 g, 3.34 mmol, 1 equiv), 6-{[(3S)-3-methylpiperidin-1-yl]methyl}-4-(trifluoromethyl)-2,3-dihydroisoindol-1-one (INT4, 1.04 g, 3.34 mmol, 1.0 equiv), Xantphos (0.39 g, 0.66 mmol, 0.2 equiv), Pd(OAc)₂ (0.08 g, 0.33 mmol, 0.1 equiv) and Cs₂CO₃ (2.18 g, 6.68 mmol, 2 equiv) in dioxane (33 mL) was stirred for 1 h at 90° C. under nitrogen atmosphere. LCMS indicated the reaction was completed. The mixture was allowed to cool down to room temperature. The resulting mixture was filtered, the filter cake was washed with MeOH (3×10 mL). The filtrate was concentrated under reduced pressure. The residue was purified by silica gel column chromatography, eluted with CH₂Cl₂/MeOH (10:1) to afford 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(trifluoromethyl)-3H-isoindol-1-one (Compound 9, 1 g, 52%) as a yellow solid. LCMS (ES, m/z): 575,577 [M+H]⁺

### Step 7: Synthesis of Compound 10

To a stirred mixture of 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(trifluoromethyl)-3H-isoindol-1-one (Compound 9, 500 mg, 0.87 mmol, 1 equiv) and 2-[(triphenylmethyl)sulfanyl]ethanamine hydrochloride (371 mg, 1.04 mmol, 1.2 equiv) in 1,4-dioxane (5 mL) were added XantPhos (100 mg, 0.17 mmol, 0.2 equiv), Cs₂CO₃ (566 mg, 1.74 mmol, 2 equiv) and Pd(AcO)₂ (29 mg, 0.13 mmol, 0.15 equiv) at room temperature under nitrogen atmosphere. The resulting mixture was stirred for overnight at 120° C. under nitrogen atmosphere. LCMS indicated the reaction was completed. The mixture was allowed to cool down to room temperature. The resulting mixture was diluted with water (50 mL). The resulting mixture was extracted with EtOAc (3×20 mL). The combined organic layers were washed with brine (50 mL), dried over anhydrous Na₂SO₄. After filtration, the filtrate was concentrated under reduced pressure. The residue was purified by silica gel column chromatography, eluted with CH₂Cl₂/MeOH (10:1) to afford 2-(4-{3-[(4-methyl-1,2,4-triazol-3-yl)methyl]oxetan-3-yl}-6-({2-[(triphenylmethyl)sulfanyl]ethyl}amino)pyridin-2-yl)-6-{[(3S)-3-methylpiperidin-1-yl]methyl}-4-(trifluoromethyl)-3H-isoindol-1-one (Compound 10, 400 mg, 53%) as a brown solid. LCMS (ESI, m/z):858[M+H]⁺

### Step 8: Synthesis of Compound 11

To a stirred solution of 2-(4-{3-[(4-methyl-1,2,4-triazol-3-yl)methyl]oxetan-3-yl}-6-({2-[(triphenylmethyl)sulfanyl]ethyl}amino)pyridin-2-yl)-6-{[(3S)-3-methylpiperidin-1-yl]methyl}-4-(trifluoromethyl)-3H-isoindol-1-one (Compound 10, 400 mg, 0.46 mmol, 1 equiv) in DCM (4 mL) was added TFA (2 mL) and tris(propan-2-yl)silane (200 uL) dropwise at room temperature. The resulting mixture was stirred for 1 h at room temperature under nitrogen atmosphere. LCMS indicated the reaction was completed. The resulting mixture was concentrated under reduced pressure. The residue was purified by reverse flash chromatography with the following conditions: column, C18 silica gel; mobile phase, MeCN in Water (0.05% TFA), 5% to 60% gradient in 30 min; detector, UV 254 nm. The collected fraction was concentrated to afford 2-(4-{3-[(4-methyl-1,2,4-triazol-3-yl)methyl]oxetan-3-yl}-6-[(2-sulfanylethyl)amino]pyridin-2-yl)-6-{[(3S)-3-methylpiperidin-1-yl]methyl}-4-(trifluoromethyl)-3H-isoindol-1-one (Compound 11, 270 mg, 94%) as a yellow solid. LCMS (ESI, m/z):616[M+H]⁺

### Step 9: Synthesis of Compound 12

To a stirred solution of 2-(4-{3-[(4-methyl-1,2,4-triazol-3-yl)methyl]oxetan-3-yl}-6-[(2-sulfanylethyl)amino]pyridin-2-yl)-6-{[(3S)-3-methylpiperidin-1-yl]methyl}-4-(trifluoromethyl)-3H-isoindol-1-one (Compound 10, 260 mg, 0.42 mmol, 1 equiv) and [(2S)-2-[(tert-butoxycarbonyl)amino]propanamido]methyl acetate (109 mg, 0.42 mmol, 1 equiv) in DCM (2.5 mL) was added TFA (25 uL) at room temperature. The resulting mixture was stirred for 1 h at room temperature under nitrogen atmosphere. LCMS indicated the reaction was completed. The resulting mixture was used in the next step directly without further purification. LCMS (ESI, m/z):815[M+H]⁺

### Step 10: Synthesis of Compound 13

To a stirred solution of tert-butyl N-[(1S)-1-{[({2-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]ethyl}sulfanyl)methyl]carbamoyl}ethyl]carbamate (Compound 12, 200 mg, 0.24 mmol, 1 equiv) in DCM (2 mL) was added TFA (1 mL) dropwise at 0° C. under air atmosphere. The resulting mixture was stirred for 1 h at room temperature under nitrogen atmosphere. LCMS indicated the reaction was completed. The resulting mixture was concentrated under reduced pressure. The residue was purified by reverse flash chromatography with the following conditions: column, C18 silica gel; mobile phase, MeCN in Water (0.05% TFA), 5% to 60% gradient in 30 min; detector, UV 254 nm. The collected fraction was concentrated to afford (2S)-2-amino-N-[({2-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]ethyl}sulfanyl)methyl]propanamide (Compound 13, 70 mg, 39%) as an off-white solid. LCMS (ESI, m/z):716[M+H]⁺

### Step 11: Synthesis of Compound 203

To a stirred solution of (2R)-2-[(2S)-2-[6-(2,5-dioxopyrrol-1-yl)hexanamido]propanamido]propanoic acid (Compound 13, 32 mg, 0.09 mmol, 1.1 equiv) and HATU (38 mg, 0.10 mmol, 1.2 equiv) in DMF (1.0 mL) was added HOBT (113 mg, 0.08 mmol, 1 equiv) at room temperature under nitrogen atmosphere. The resulting mixture was stirred for 30 min at room temperature under nitrogen atmosphere. To the above mixture was added (2S)-2-amino-N-[({2-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]ethyl}sulfanyl)methyl]propanamide (60 mg, 0.08 mmol, 1 equiv) and DIEA (9 mg, 0.067 mmol, 0.8 equiv) at 0° C. The resulting mixture was stirred for additional overnight at room temperature. LCMS indicated the reaction was completed. The reaction mixture was purified by reverse flash chromatography with the following conditions: column, C18 silica gel; mobile phase, ACN in water (0.1% FA), 5% to 60% gradient in 30 min; detector, UV 254 nm. The collected fraction was concentrated to afford crude product (15 mg). The crude product (15 mg) was purified by Prep-HPLC with the following conditions (Column: XBridge Prep C18 OBD Column, 19*150 mm, 5 m; Mobile Phase A: Water (0.1% FA), Mobile Phase B: ACN; Flow rate: 25 mL/min; Gradient: 23% B to 53% B in 7 min, 53% B; Wave Length: 254 nm; RT1 (min): 5; The collected fraction was lyophilized to afford 6-(2,5-dioxopyrrol-1-yl)-N-[(1S)-1-{[(1R)-1-{[(1S)-1-{[({2-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]ethyl}sulfanyl)methyl]carbamoyl}ethyl]carbamoyl}ethyl]carbamoyl}ethyl]hexanamide; formic acid (Compound 203, 8.8 mg, 9%) as a white solid. LCMS (ESI, ms):1051[M+H−FA]⁺; ¹H NMR (300 MHz, DMSO-d₆) δ 8.45-8.44 (m, 1H), 8.25 (s, 1H), 8.15-7.97 (m, 6H), 7.42 (s, 1H) 6.97 (s, 2H), 6.81 (br s, 1H), 6.03 (s, 1H), 5.19 (s, 2H), 4.89 (d, J=6.0 Hz, 2H), 4.77 (d, J=6.0 Hz, 2H), 4.27 (d, J=6.0 Hz, 2H), 4.15-4.09 (m, 3H), 3.24-3.11 (m, 7H), 2.75-2.65 (m, 3H), 2.17-2.06 (m, 3H), 1.83-1.35 (m, 11H), 1.30-1.07 (m, 14H), 0.90-0.84 (m, 4H).

### Synthesis of Compound 204

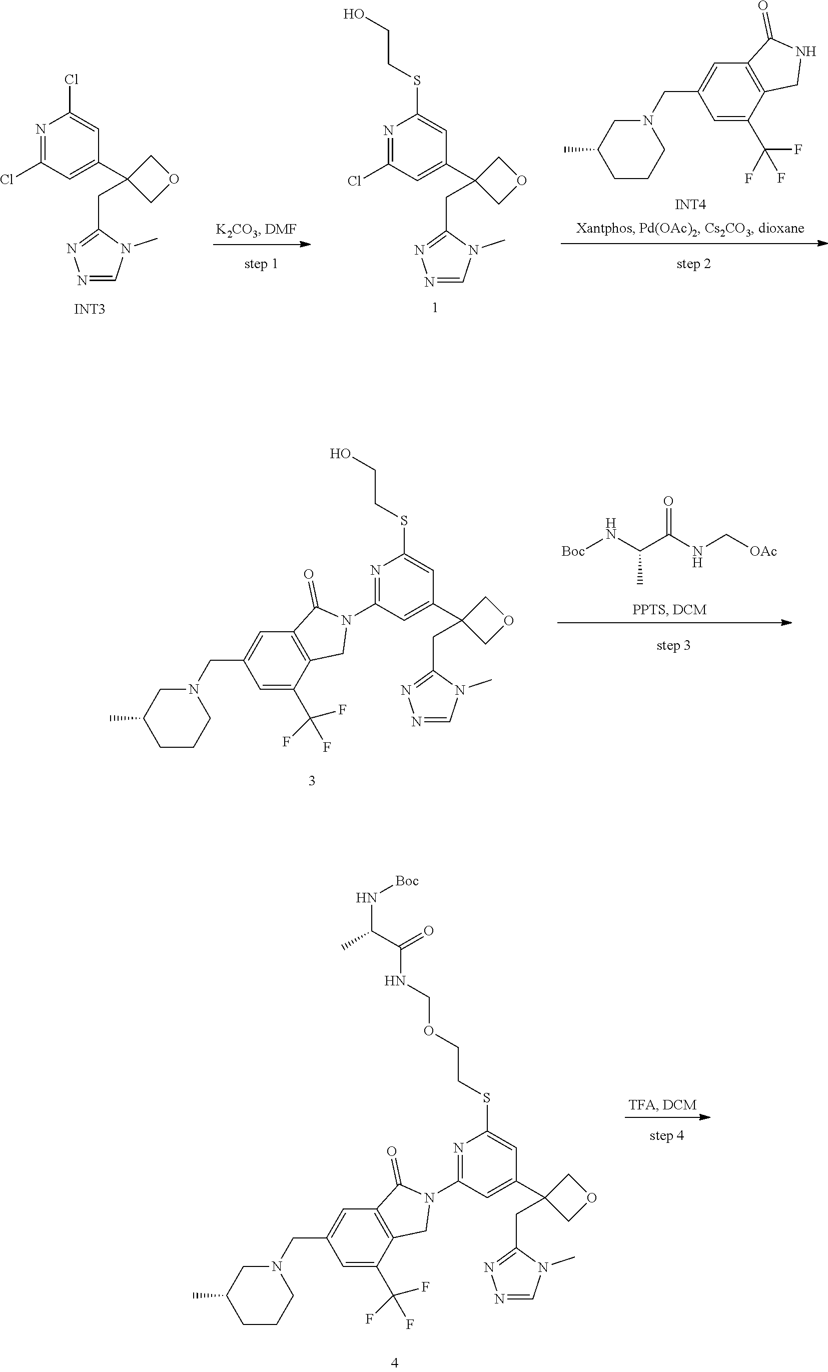

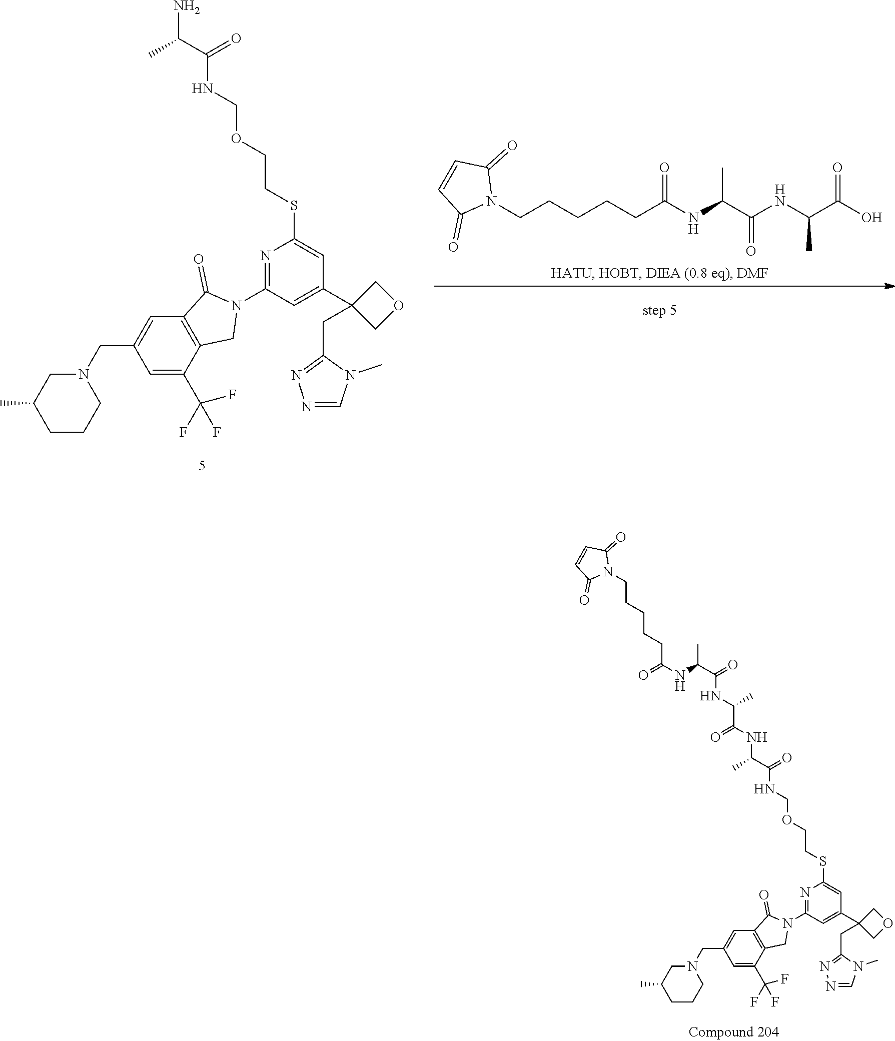 [SMILES: C[C@H]1CCCN(Cc2cc3c(c(C(F)(F)F)c2)CN(c2cc(C4(Cc5nncn5C)COC4)cc(SCCOCNC(=O)[C@H](C)N)n2)C3=O)C1 ; C[C@H](NC(=O)CCCCCN1C(=O)C=CC1=O)C(=O)N[C@H](C)C(=O)O ; C[C@H]1CCCN(Cc2cc3c(c(C(F)(F)F)c2)CN(c2cc(C4(Cc5nncn5C)COC4)cc(SCCOCNC(=O)[C@H](C)NC(=O)[C@@H](C)NC(=O)[C@H](C)NC(=O)CCCCCN4C(=O)C=CC4=O)n2)C3=O)C1]

### Step 1: Synthesis of Compound 1

To a stirred mixture of 2,6-dichloro-4-{3-[(4-methyl-1,2,4-triazol-3-yl)methyl]oxetan-3-yl}pyridine (INT3, 1000 mg, 3.34 mmol, 1 equiv) and β-mercaptoethanol (263 mg, 3.37 mmol, 1.01 equiv) in DMF (10 mL) was added K₂CO₃ (928 mg, 6.71 mmol, 2.01 equiv) in portions at room temperature. The resulting mixture was stirred for 2 h at 70° C. under nitrogen atmosphere. LCMS indicated the reaction was completed. The reaction mixture was filtered and the solid was washed with EA. The filtrate was concentrated to dryness under vacuum. The residue was purified by silica gel column chromatography, eluted with CH₂Cl₂/MeOH (9:1) to afford 2-[(6-chloro-4-{3-[(4-methyl-1,2,4-triazol-3-yl)methyl]oxetan-3-yl}pyridin-2-yl)sulfanyl]ethanol (Compound 1, 1.1 g, 88%) a white solid. LCMS:(ES·m/z):341,343[M+1]⁺

### Step 2: Synthesis of Compound 3

To a stirred mixture of 2-[(6-chloro-4-{3-[(4-methyl-1,2,4-triazol-3-yl)methyl]oxetan-3-yl}pyridin-2-yl)sulfanyl]ethanol (Compound 1, 370 mg, 1.08 mmol, 1 equiv) and 6-{[(3S)-3-methylpiperidin-1-yl]methyl}-4-(trifluoromethyl)-2,3-dihydroisoindol-1-one (INT4, 369 mg, 1.18 mmol, 1.09 equiv) in dioxane (8 mL) was added Xantphos (125 mg, 0.21 mmol, 0.20 equiv) and Pd(OAc)₂ (24 mg, 0.10 mmol, 0.10 equiv) and Cs₂CO₃ (1057 mg, 3.24 mmol, 2.99 equiv) in portions at room temperature. The resulting mixture was stirred for 1 h at 120° C. under nitrogen atmosphere. LCMS indicated the reaction was completed. The resulting mixture was allowed to cool down to room temperature and concentrated under reduced pressure. The residue was purified by silica gel column chromatography, eluted with CH₂Cl₂/MeOH (8:1) to afford 2-{6-[(2-hydroxyethyl)sulfanyl]-4-{3-[(4-methyl-1,2,4-triazol-3-yl)methyl]oxetan-3-yl}pyridin-2-yl}-6-{[(3S)-3-methylpiperidin-1-yl]methyl}-4-(trifluoromethyl)-3H-isoindol-1-one (Compound 3, 380 mg, 56%) as a light brown solid. LCMS:(ES·m/z):617[M+1]; ¹H NMR (300 MHz, DMSO-d₆) δ 8.26 (s, 1H), 8.04 (s, 1H), 7.98 (s, 1H), 7.94 (s, 1H), 6.96 (s, 1H), 5.22 (s, 2H), 3.94-3.90 (m, 3H), 3.83-3.82 (m, 2H), 3.66-3.65 (m, 2H), 3.57 (s, 2H), 3.11 (s, 2H), 3.25 (d, J=9.6 Hz, 2H), 2.80-2.65 (m, 2H), 2.00-1.90 (m, 1H), 1.77-1.55 (m, 4H), 1.55-1.40 (m, 1H), 0.82-0.81 (m, 4H).

### Step 3: Synthesis of Compound 4

To a stirred mixture of 2-{6-[(2-hydroxyethyl)sulfanyl]-4-{3-[(4-methyl-1,2,4-triazol-3-yl)methyl]oxetan-3-yl}pyridin-2-yl}-6-{[(3S)-3-methylpiperidin-1-yl]methyl}-4-(trifluoromethyl)-3H-isoindol-1-one (Compound 3, 240 mg, 0.38 mmol, 1 equiv) and [(2S)-2-[(tert-butoxycarbonyl)amino]propanamido]methyl acetate (202 mg, 0.77 mmol, 2 equiv) in DCM (7 mL) was added PTSA (134 mg, 0.77 mmol, 2 equiv) in portions at room temperature under nitrogen atmosphere. The resulting mixture was stirred for overnight at 50° C. under nitrogen atmosphere. The mixture was allowed to cool down to room temperature. LCMS indicated the reaction was ˜40% Product. The reaction mixture was purified by reverse flash chromatography with the following conditions: column, C18 silica gel; mobile phase, ACN in water, 5% to 50% gradient in 40 min; detector, UV 254 nm. This resulted in tert-butyl N-[(1S)-1-[({2-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)sulfanyl]ethoxy}methyl)carbamoyl]ethyl]carbamate (Compound 4, 150 mg, 37%) as a brown solid. LCMS:(ES·m/z):817[M+1]⁺.

### Step 4: Synthesis of Compound 5

To a stirred mixture of tert-butyl N-[(1S)-1-[({2-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)sulfanyl]ethoxy}methyl)carbamoyl]ethyl]carbamate (Compound 4, 150 mg, 0.18 mmol, 1 equiv) in DCM (5 mL) was added TFA (2.00 mL) dropwise at 0° C. under. The resulting mixture was stirred for 2 h at 0° C. LCMS indicated the reaction was completed. The resulting mixture was concentrated under reduced pressure. The residue was purified by reverse flash chromatography with the following conditions: column, C18 silica gel; mobile phase, ACN in water (0.05% TFA), 0% to 40% gradient in 40 min; detector, UV 254 nm. The collected fraction was lyophilized to afford (2S)-2-amino-N-({2-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)sulfanyl]ethoxy}methyl)propanamide (Compound 5, 110 mg, 79%) as a yellow solid. LCMS:717 (M+H)+

### Step 5: Synthesis of Compound 204

To a stirred mixture of (2R)-2-[(2S)-2-[6-(2,5-dioxopyrrol-1-yl)hexanamido]propanamido]propanoic acid (Compound 5, 21 mg, 0.060 mmol, 1.22 equiv) and HATU (28 mg, 0.074 mmol, 1.51 equiv) in DMF (2 mL) were added (2S)-2-amino-N-({2-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)sulfanyl]ethoxy}methyl)propanamide (Compound 5, 35 mg, 0.049 mmol, 1 equiv) and DIEA (4.67 mg, 0.036 mmol, 0.74 equiv) at 0° C. under nitrogen atmosphere. The resulting mixture was stirred for 1 h at room temperature. LCMS indicated the reaction was completed. The reaction mixture was purified by reverse flash chromatography with the following conditions: column, C18 silica gel; mobile phase, ACN in water (0.1% FA), 5% to 50% gradient in 40 min; detector, UV 254 nm. The collected fraction was lyophilized to dryness. The crude product was re-purified by following condition: Column: XSelect CSH Prep C18 OBD Column, 19×250 mm, 5 m; Mobile Phase A: Water (0.1% FA), Mobile Phase B: ACN; Flow rate: 25 mL/min; Gradient: 16% B to 46% B in 7 min, 46% B; Wave Length: 254 nm; RT1 (min): 6 min; The collected fraction was lyophilized to afford 6-(2,5-dioxopyrrol-1-yl)-N-[(1S)-1-{[(1R)-1-{[(1S)-1-[({2-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)sulfanyl]ethoxy}methyl)carbamoyl]ethyl]carbamoyl}ethyl]carbamoyl}ethyl]hexanamide (Compound 204, 5.8 mg, 11%) as an off-white solid. LCMS:(ms, ESI): 1052 (M+H)*; ¹H NMR (400 MHz, DMSO-d₆) δ 8.60 (t, J=6.7 Hz, 1H), 8.27 (s, 1H), 8.24-8.10 (m, 2H), 8.10-7.91 (m, 4H), 6.97-6.96 (m, 3H), 5.24 (s, 2H), 4.90-4.82 (m, 4H), 4.61-4.59 (m, 2H), 4.27-4.09 (m, 3H), 3.74-3.57 (m, 5H), 3.35-3.30 (m, 8H), 2.72 (t, J=10.1 Hz, 2H), 2.06 (t, J=7.4 Hz, 2H), 1.96-1.94 (m, 1H), 1.75-1.55 (m, 4H), 1.53-1.38 (m, 5H), 1.17-1.15 (m, 11H), 0.82-0.81 (m, 4H).

### Synthesis of Compound 205

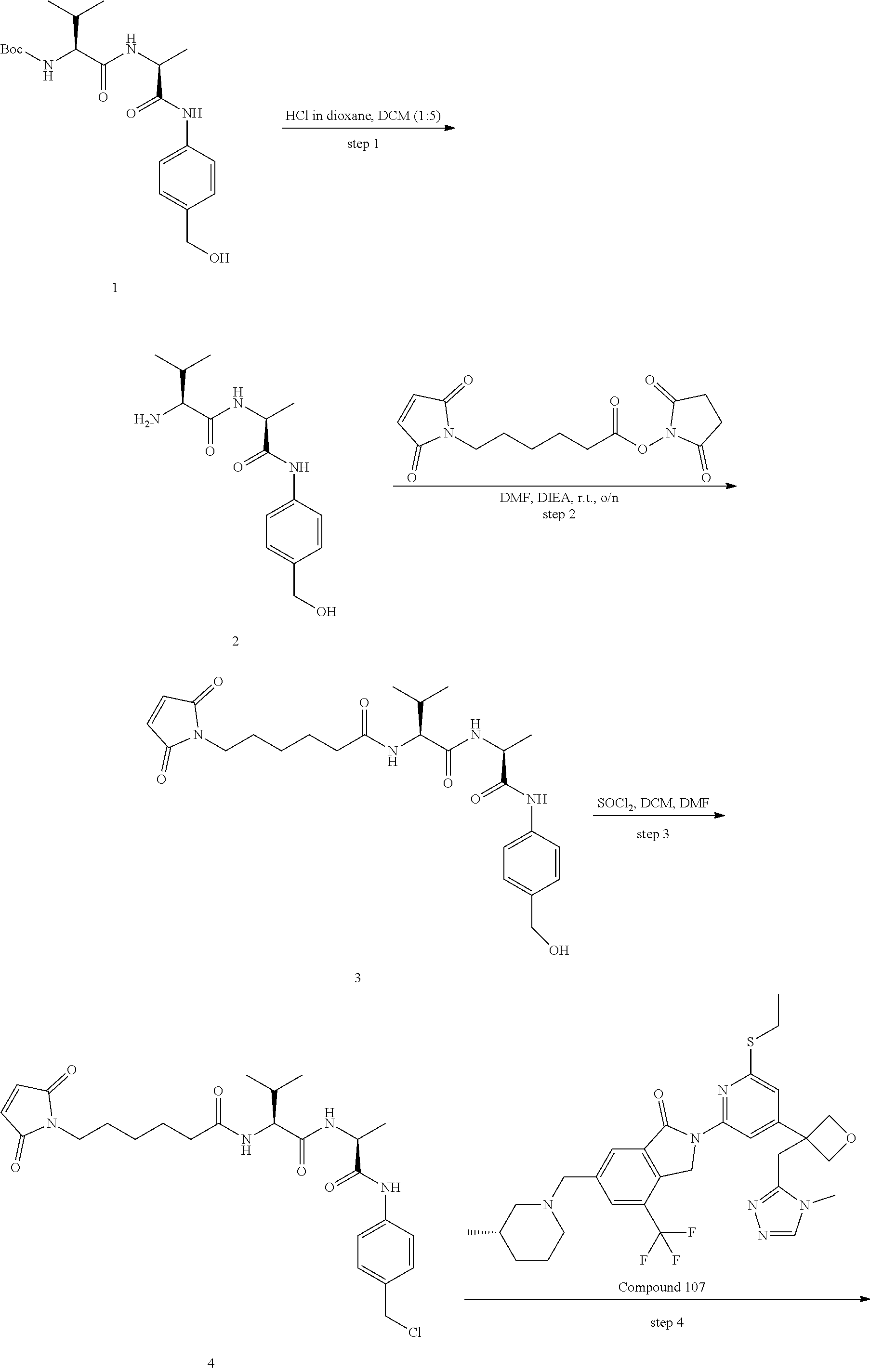 [SMILES: CC(C)[C@H](NC(=O)OC(C)(C)C)C(=O)N[C@@H](C)C(=O)Nc1ccc(CO)cc1 ; CC(C)[C@H](N)C(=O)N[C@@H](C)C(=O)Nc1ccc(CO)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(CO)cc1 ; CC(C)[C@H](NC(=O)CCCCCN1C(=O)C=CC1=O)C(=O)N[C@@H](C)C(=O)Nc1ccc(CCl)cc1 ; CCSc1cc(C2(Cc3nncn3C)COC2)cc(N2Cc3c(cc(CN4CCC[C@H](C)C4)cc3C(F)(F)F)C2=O)n1]

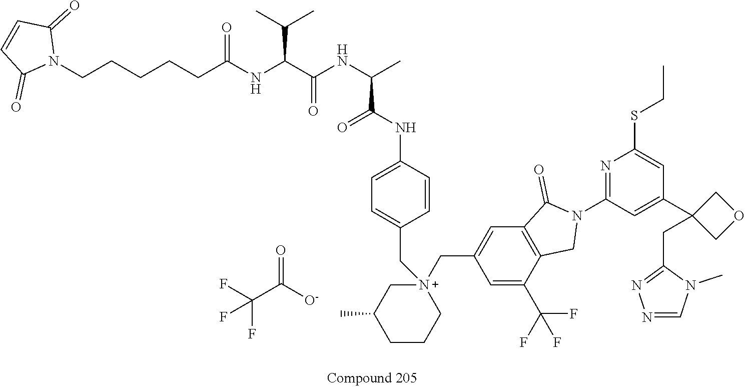 [SMILES: CCSc1cc(C2(Cc3nncn3C)COC2)cc(N2Cc3c(cc(C[N+]4(Cc5ccc(NC(=O)[C@H](C)NC(=O)[C@@H](NC(=O)CCCCCN6C(=O)C=CC6=O)C(C)C)cc5)CCC[C@H](C)C4)cc3C(F)(F)F)C2=O)n1 ; O=C([O-])C(F)(F)F]

### Step 1: Synthesis of Compound 2

To a stirred solution of tert-butyl N-[(1S)-1-{[(1S)-1-{[4-(hydroxymethyl)phenyl]carbamoyl}ethyl]carbamoyl}-2-methylpropyl]carbamate (Compound 1, 400 mg, 1.01 mmol, 1 equiv) in DCM (5 mL) was added HCl (g, 4N in dioxane, 1 mL) dropwise at 0° C. under air atmosphere. The resulting mixture was stirred for 2 h at room temperature under air atmosphere. LCMS indicated the reaction was completed. The resulting mixture was concentrated under reduced pressure. The residue was re-dissolved in DCM (5 mL) and concentrated under reduced pressure. The crude product mixture was used in the next step directly without further purification. LCMS:(ES·m/z):294[M+1]⁺.

### Step 2: Synthesis of Compound 3

To a stirred mixture of (2S)-2-amino-N-[(1S)-1-{[4-(hydroxymethyl)phenyl]carbamoyl}ethyl]-3-methylbutanamide (Compound 2, 400 mg, 1.36 mmol, 1 equiv) and 2,5-dioxopyrrolidin-1-yl 6-(2,5-dioxopyrrol-1-yl)hexanoate (504 mg, 1.63 mmol, 1.2 equiv) in DMF (5 mL) was added DIEA (352 mg, 2.72 mmol, 2 equiv) dropwise at 0° C. The resulting mixture was stirred for 12 h at room temperature under air atmosphere. LCMS indicated the reaction was completed. The reaction mixture was purified by reverse flash chromatography with the following conditions: column, silica gel; mobile phase, ACN in water (0.05% TFA), 5% to 50% gradient in 30 min; detector, UV 254 nm. The collected fraction was concentrated to afford 6-(2,5-dioxopyrrol-1-yl)-N-[(1S)-1-{[(1S)-1-{[4-(hydroxymethyl)phenyl]carbamoyl}ethyl]carbamoyl}-2-methylpropyl]hexanamide (Compound 3, 165 mg, 24%) as a yellow solid. LCMS:(ES·m/z):487[M+1]⁺.

### Step 3: Synthesis of Compound 4

To a stirred solution of 6-(2,5-dioxopyrrol-1-yl)-N-[(1S)-1-{[(1S)-1-{[4-(hydroxymethyl)phenyl]carbamoyl}ethyl]carbamoyl}-2-methylpropyl]hexanamide (Compound 3, 160 mg, 0.32 mmol, 1 equiv) in DCM (2 mL) was added SOCl₂ (78 mg, 0.65 mmol, 2.00 equiv) dropwise at 0° C. The resulting mixture was stirred for 2 h at room temperature under air atmosphere. LCMS indicated the reaction was completed. The resulting mixture was concentrated under reduced pressure. This resulted in N-[(1S)-1-{[(1S)-1-{[4-(chloromethyl)phenyl]carbamoyl}ethyl]carbamoyl}-2-methylpropyl]-6-(2,5-dioxopyrrol-1-yl)hexanamide (Compound 4, 135 mg, 75%) as a white solid. The crude product was used in the next step directly without further purification. LCMS:(ES·m/z):505,507[M+1]⁺.

### Step 4: Synthesis of Compound 205

To a stirred mixture of N-[(1S)-1-{[(1S)-1-{[4-(chloromethyl)phenyl]carbamoyl}ethyl]carbamoyl}-2-methylpropyl]-6-(2,5-dioxopyrrol-1-yl)hexanamide (Compound 4, 129 mg, 0.25 mmol, 1.2 equiv) and 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-4-(trifluoromethyl)-3H-isoindol-1-one (Compound 107, 128 mg, 0.21 mmol, 1.00 equiv) in DMF (1 mL) was added DIEA (55 mg, 0.42 mmol, 2 equiv) dropwise at 0° C. The resulting mixture was stirred for 2 days at 50° C. ˜15% desired product can be found on LCMS. The residue was purified by reverse flash chromatography with the following conditions: column, C18 silica gel; mobile phase, ACN in water (0.05% TFA), 5% to 60% gradient in 40 min; detector, UV 254 nm. This result in the crude product (60 mg). The crude product (60 mg) was re-purified by Prep-HPLC with the following conditions (Column: XBridge Shield RP18 OBD Column, 30*150 mm, 5 m; Mobile Phase A: Water (0.05% TFA), Mobile Phase B: ACN; Flow rate: 60 mL/min; Gradient: 27% B to 52% B in 7 min, 52% B; Wave Length: 254 nm; RT1 (min): 6.13; The collected fraction was lyophilized to afford (3S)-1-({4-[(2S)-2-[(2S)-2-[6-(2,5-dioxopyrrol-1-yl)hexanamido]-3-methylbutanamido]propanamido]phenyl}methyl)-1-({2-[6-(ethylsulfanyl)-4-{3-[(4-methyl-1,2,4-triazol-3-yl)methyl]oxetan-3-yl}pyridin-2-yl]-3-oxo-7-(trifluoromethyl)-1H-isoindol-5-yl}methyl)-3-methylpiperidin-1-ium (Compound 205, 12.9 mg, 6%) as a white solid. LCMS:(ES·m/z):1069[M+1-TFA]⁺; ¹H NMR (400 MHz, DMSO-d₆) δ10.4 (br s, 1H), 8.7-8.4 (m, 1H), 8.26-8.22 (m, 3H), 8.06 (s, 1H), 7.81-7.79 (m, 3H), 7.54 (s, 2H), 6.97 (s, 3H), 5.29 (s, 2H), 4.91-4.90 (m, 2H), 4.84-4.82 (m, 3H), 4.67-4.55 (m, 2H), 4.47-4.38 (m, 1H), 4.2-4.13 (m, 1H), 3.57-3.52 (m, 4H), 3.39-3.36 (m, 5H), 3.14-3.13 (m, 2H), 2.98-2.80 (m, 1H), 2.49-2.48 (m, 1H), 2.40-2.27 (m, 1H), 2.26-1.88 (m, 4H), 1.82-1.61 (m, 2H), 1.49-1.47 (m, 4H), 1.35-1.31 (m, 6H), 1.28-1.13 (m, 3H), 0.88-0.82 (m, 10H).

### Synthesis of Compound 206

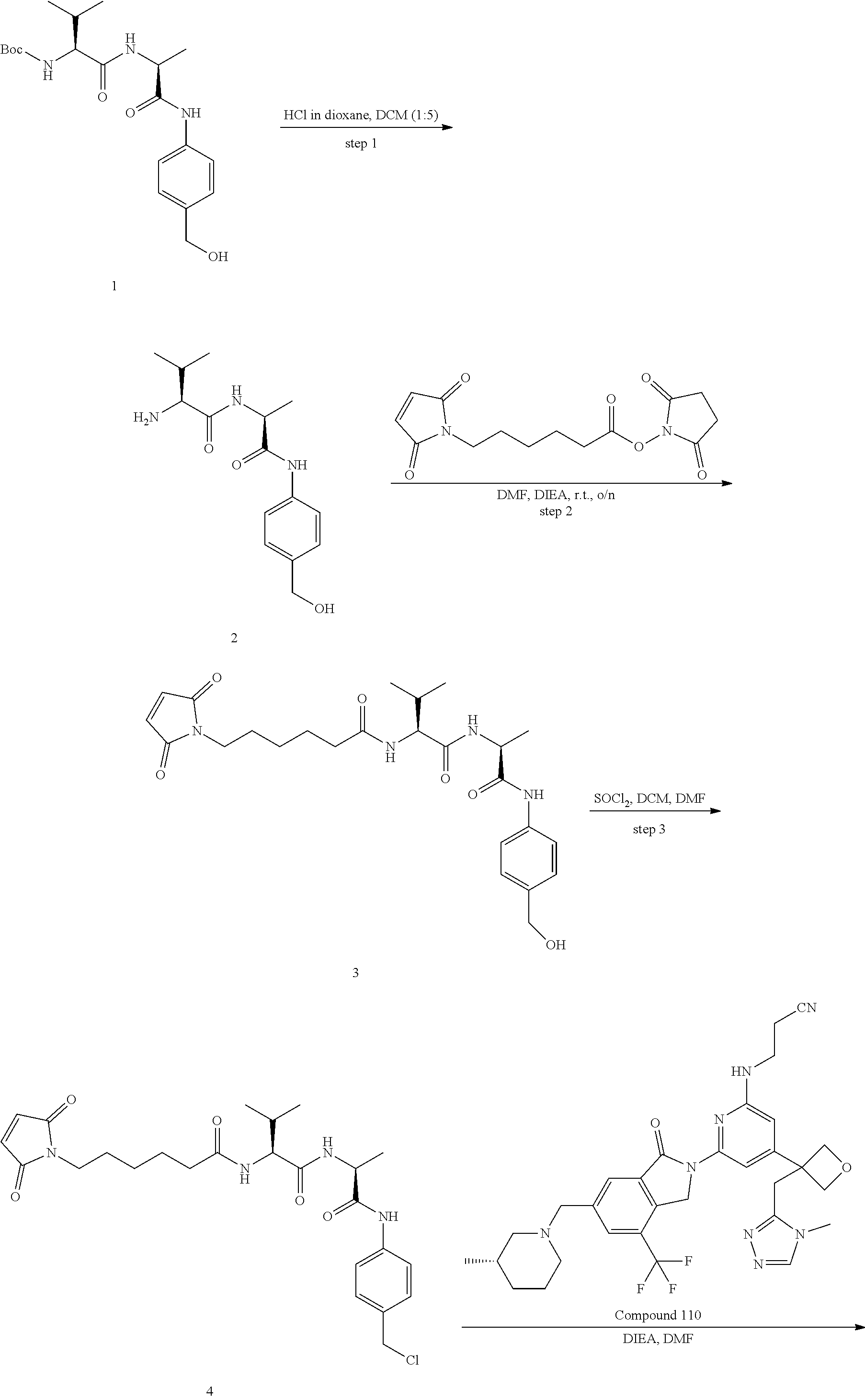 [SMILES: CC(C)[C@H](NC(=O)OC(C)(C)C)C(=O)N[C@@H](C)C(=O)Nc1ccc(CO)cc1 ; CC(C)[C@H](N)C(=O)N[C@@H](C)C(=O)Nc1ccc(CO)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(CO)cc1 ; CC(C)[C@H](NC(=O)CCCCCN1C(=O)C=CC1=O)C(=O)N[C@@H](C)C(=O)Nc1ccc(CCl)cc1 ; C[C@H]1CCCN(Cc2cc3c(c(C(F)(F)F)c2)CN(c2cc(C4(Cc5nncn5C)COC4)cc(NCCC#N)n2)C3=O)C1]

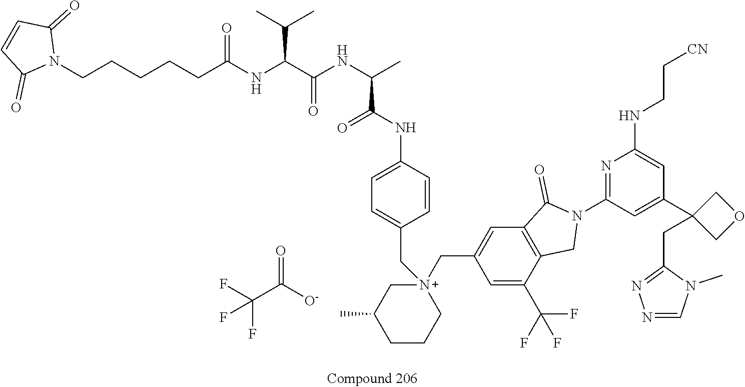 [SMILES: CC(C)[C@H](NC(=O)CCCCCN1C(=O)C=CC1=O)C(=O)N[C@@H](C)C(=O)Nc1ccc(C[N+]2(Cc3cc4c(c(C(F)(F)F)c3)CN(c3cc(C5(Cc6nncn6C)COC5)cc(NCCC#N)n3)C4=O)CCC[C@H](C)C2)cc1 ; O=C([O-])C(F)(F)F]

### Step 1. Synthesis of Compound 2

To a stirred solution of tert-butyl N-[(1S)-1-{[(1S)-1-{[4-(hydroxymethyl)phenyl]carbamoyl}ethyl]carbamoyl}-2-methylpropyl]carbamate (Compound 1, 400 mg, 1.01 mmol, 1 equiv) in DCM (5 mL) was added HCl (g, 4N in dioxane, 400 uL) in portions at 0° C. The resulting mixture was stirred for 2 h at room temperature under air atmosphere. LCMS indicated the reaction was completed. The resulting mixture was concentrated under vacuum. This resulted in (2S)-2-amino-N-[(1S)-1-{[4-(hydroxymethyl)phenyl]carbamoyl}ethyl]-3-methylbutanamide (Compound 2, 270 mg, 90%) as a yellow solid. The crude product mixture was used in the next step directly without further purification. LCMS:(ESI, m/z):294[M+1]⁺.

### Step 2: Synthesis of Compound 3

To a stirred mixture of (2S)-2-amino-N-[(1S)-1-{[4-(hydroxymethyl)phenyl]carbamoyl}ethyl]-3-methylbutanamide (Compound 2, 300 mg, 1.02 mmol, 1 equiv) and 2,5-dioxopyrrolidin-1-yl 6-(2,5-dioxopyrrol-1-yl)hexanoate (346 mg, 1.12 mmol, 1.1 equiv) in DMF (1 mL) was added DIEA (264 mg, 2.04 mmol, 2 equiv) dropwise at room temperature. The resulting mixture was stirred for overnight at room temperature under air atmosphere. LCMS indicated the reaction was completed. The reaction mixture was purified by reverse flash chromatography with the following conditions: column, C18 silica gel; mobile phase, ACN in water (0.05% TFA), 0% to 50% gradient in 35 min; detector, UV 254 nm. This resulted in 6-(2,5-dioxopyrrol-1-yl)-N-[(1S)-1-{[(1S)-1-{[4-(hydroxymethyl)phenyl]carbamoyl}ethyl]carbamoyl}-2-methylpropyl]hexanamide (Compound 3, 135 mg, 26%) as a yellow solid. LCMS:(ESI, m/z):487[M+1]⁺.

### Step 3: Synthesis of Compound 4

To a stirred solution of 6-(2,5-dioxopyrrol-1-yl)-N-[(1S)-1-{[(1S)-1-{[4-(hydroxymethyl)phenyl]carbamoyl}ethyl]carbamoyl}-2-methylpropyl]hexanamide (Compound 3, 125 mg, 0.25 mmol, 1 equiv) in DCM (2 mL) was added SOCl₂ (61 mg, 0.51 mmol, 2 equiv) dropwise at 0° C. under air atmosphere. The resulting mixture was stirred for 2 h at room temperature. LCMS indicated the reaction was completed. The resulting mixture was concentrated under reduced pressure. The crude product was used in the next step directly without further purification. This resulted in N-[(1S)-1-{[(1S)-1-{[4-(chloromethyl)phenyl]carbamoyl}ethyl]carbamoyl}-2-methylpropyl]-6-(2,5-dioxopyrrol-1-yl)hexanamide (Compound 4, 120 mg, 83%) as a yellow solid. LCMS:(ESI, m/z): 505,507[M+1].

### Step 4: Synthesis of Compound 206

To a stirred mixture of N-[(1S)-1-{[(1S)-1-{[4-(chloromethyl)phenyl]carbamoyl}ethyl]carbamoyl}-2-methylpropyl]-6-(2,5-dioxopyrrol-1-yl)hexanamide (Compound 4, 99 mg, 0.19 mmol, 1.2 equiv) and 3-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]propanenitrile (Compound 110, 100 mg, 0.16 mmol, 1.00 equiv) in DMF (1 mL) was added DIEA (84 mg, 0.65 mmol, 4 equiv) dropwise at 0° C. under air atmosphere. The resulting mixture was stirred for overnight at room temperature under nitrogen atmosphere. LCMS indicated the reaction was 28% product and 46% the raw material. The residue was purified by reverse flash chromatography with the following conditions: column, C18 silica gel; mobile phase, ACN in water (0.05% TFA), 0% to 50% gradient in 10 min; detector, UV 254 nm. The crude product was re-purified with the following condition: Column: XSelect CSH Prep C18 OBD Column, 19*250 mm, 5 m; Mobile Phase A: Water (0.05% TFA), Mobile Phase B: ACN; Flow rate: 25 mL/min; Gradient: 25% B to 55% B in 7 min, 55% B; Wave Length: 254 nm; RT1 (min): 5; The collected fraction was lyophilized to afford (3S)-1-[(2-{6-[(2-cyanoethyl)amino]-4-{3-[(4-methyl-1,2,4-triazol-3-yl)methyl]oxetan-3-yl}pyridin-2-yl}-3-oxo-7-(trifluoromethyl)-1H-isoindol-5-yl)methyl]-1-({4-[(2S)-2-[(2S)-2-[6-(2,5-dioxopyrrol-1-yl)hexanamido]-3-methylbutanamido]propanamido]phenyl}methyl)-3-methylpiperidin-1-ium trifluoroacetate (7 mg, 4%) as a white solid. LCMS:(ES·m/z):1077[M+1-TFA]⁺; ¹H NMR (400 MHz, DMSO-d₆) δ10.21 (s, 1H), 8.33-8.20 (m, 4H), 7.79-7.77 (m, 3H), 7.52-7.49 (m, 3H), 7.25-7.07 (m, 1H), 7.00 (s, 2H), 6.08 (s, 1H), 5.27 (s, 2H), 4.91-4.90 (m, 2H), 4.79-4.77 (m, 4H), 4.62-4.48 (m, 2H), 4.47-4.13 (m, 3H), 3.53-3.50 (m, 6H), 3.28-3.20 (m, 5H), 2.92-2.80 (m, 3H), 2.28-1.62 (m, 7H), 1.53-1.47 (m, 4H), 1.34-1.32 (m, 3H), 1.28-1.15 (m, 3H), 0.89-0.83 (m, 9H)

### Synthesis of Compound 207

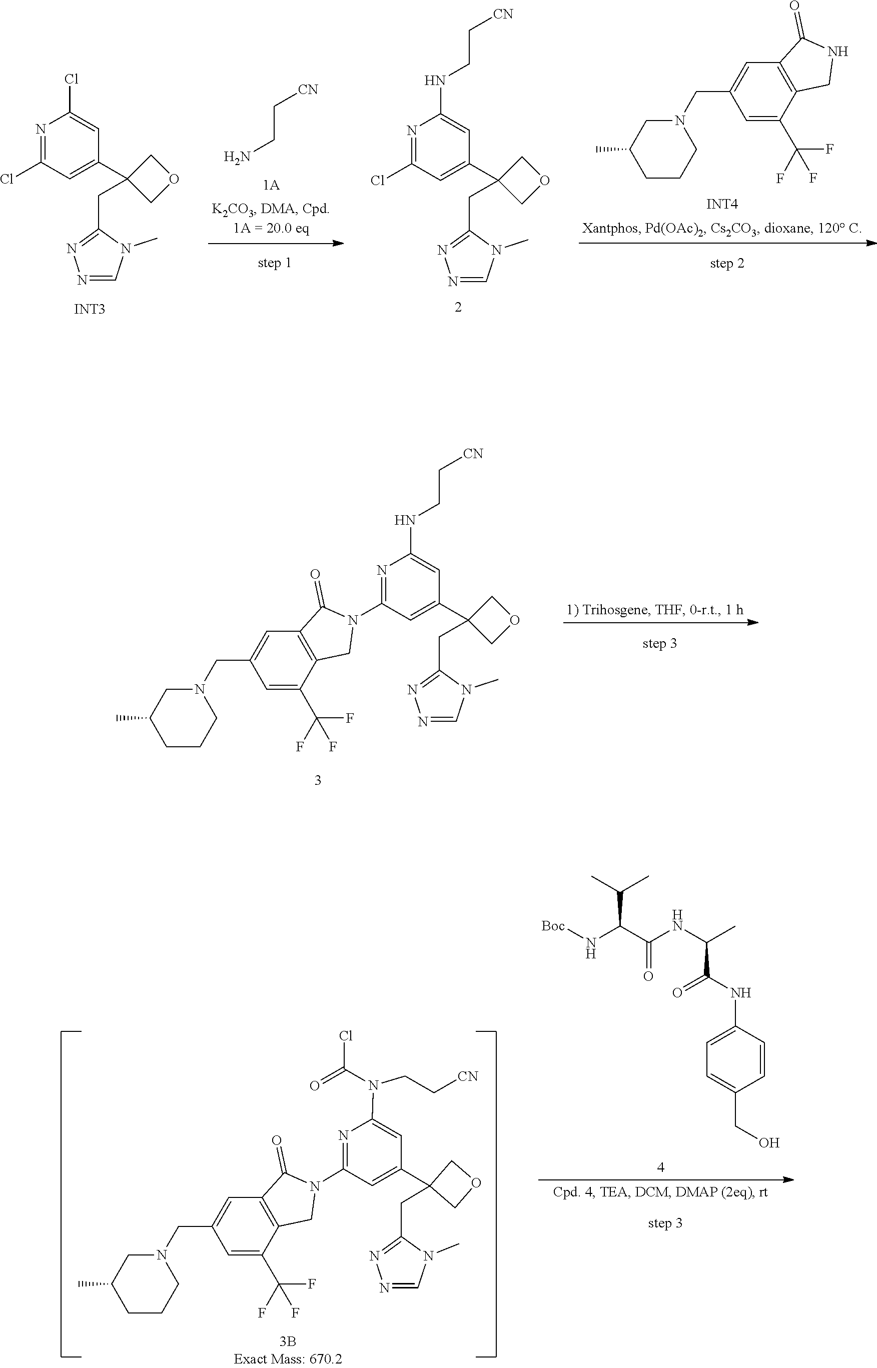

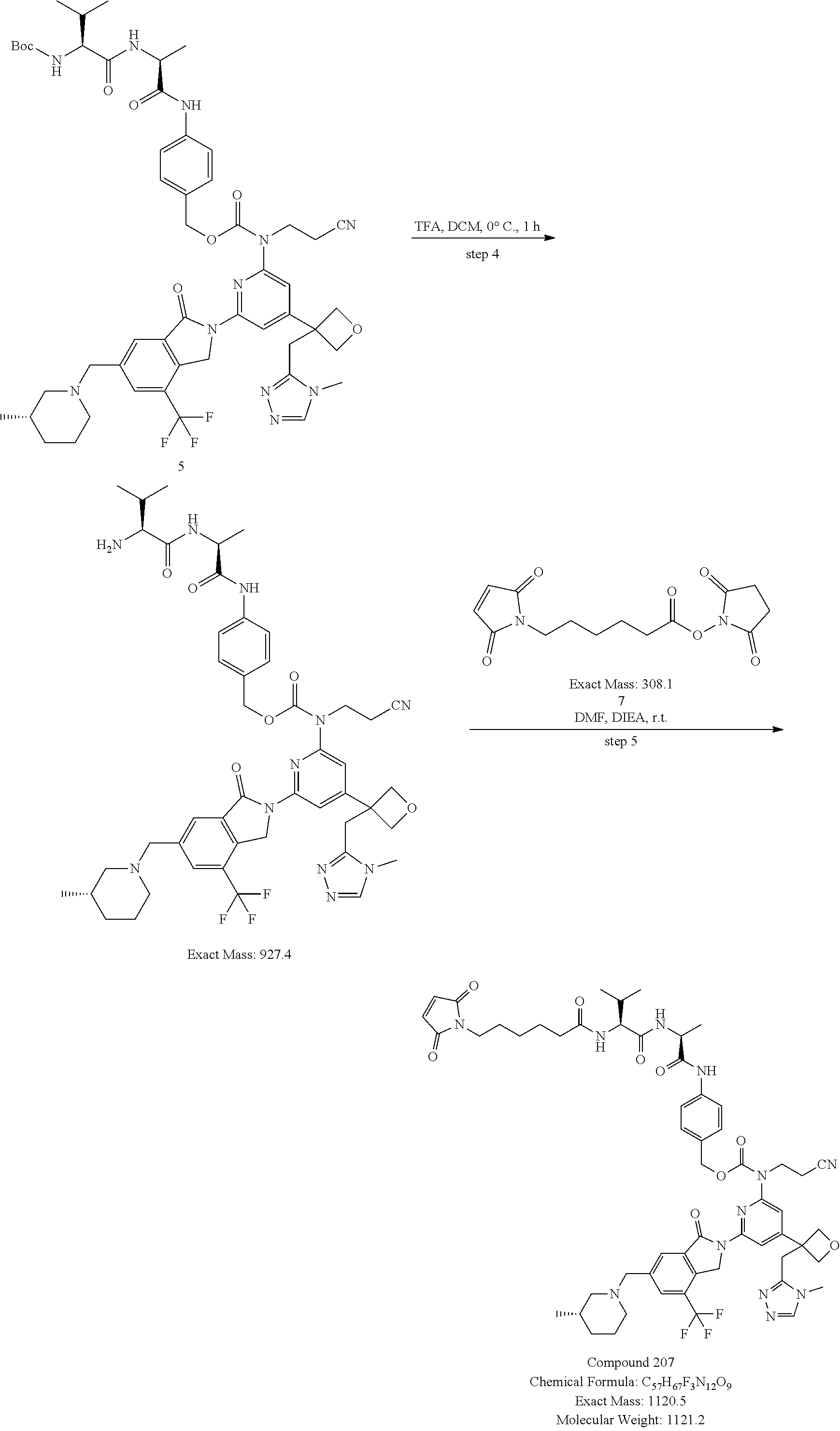 [SMILES: CC(C)[C@H](NC(=O)OC(C)(C)C)C(=O)N[C@@H](C)C(=O)Nc1ccc(COC(=O)N(CCC#N)c2cc(C3(Cc4nncn4C)COC3)cc(N3Cc4c(cc(CN5CCC[C@H](C)C5)cc4C(F)(F)F)C3=O)n2)cc1 ; CC(C)[C@H](N)C(=O)N[C@@H](C)C(=O)Nc1ccc(COC(=O)N(CCC#N)c2cc(C3(Cc4nncn4C)COC3)cc(N3Cc4c(cc(CN5CCC[C@H](C)C5)cc4C(F)(F)F)C3=O)n2)cc1 ; O=C(CCCCCN1C(=O)C=CC1=O)ON1C(=O)CCC1=O ; CC(C)[C@H](NC(=O)CCCCCN1C(=O)C=CC1=O)C(=O)N[C@@H](C)C(=O)Nc1ccc(COC(=O)N(CCC#N)c2cc(C3(Cc4nncn4C)COC3)cc(N3Cc4c(cc(CN5CCC[C@H](C)C5)cc4C(F)(F)F)C3=O)n2)cc1]

### Step 1: Synthesis of Compound 2

To a stirred solution of 2,6-dichloro-4-{3-[(4-methyl-1,2,4-triazol-3-yl)methyl]oxetan-3-yl}pyridine (INT3, 1.0 g, 3.34 mmol, 1.0 equiv) and β-aminopropionitrile (Compound 1A, 4.69 g, 66.90 mmol, 20.0 equiv) in DMA (4.0 mL) was added K₂CO₃ (0.92 g, 6.68 mmol, 2.0 equiv) in portion at room temperature under nitrogen atmosphere. The resulting mixture was stirred for two days at 120° C. under nitrogen atmosphere. ˜70% desired product could be by LCMS. The reaction was cooled to room temperature and quenched with water at room temperature. The aqueous layer was extracted with CH₂Cl₂ (3×100 mL). The resulting mixture was concentrated under vacuum. The residue was purified by silica gel column chromatography, eluted with CH₂Cl₂/MeOH (5:1) to afford 3-[(6-chloro-4-{3-[(4-methyl-1,2,4-triazol-3-yl)methyl]oxetan-3-yl}pyridin-2-yl)amino]propanenitrile (Compound 2, 600 mg, 49%) as a light green solid. LCMS: (ES, m/s): 333,335 [M+H]⁺.

### Step 2: Synthesis of Compound 3

To a stirred solution of 6-{[(3S)-3-methylpiperidin-1-yl]methyl}-4-(trifluoromethyl)-2,3-dihydroisoindol-1-one (Compound 2, 187 mg, 0.60 mmol, 1.0 equiv) and 3-[(6-chloro-4-{3-[(4-methyl-1,2,4-triazol-3-yl)methyl]oxetan-3-yl}pyridin-2-yl)amino]propanenitrile (INT4, 200 mg, 0.60 mmol, 1.0 equiv) in dioxane (8.0 mL) was added Xantphos (139 mg, 0.24 mmol, 0.4 equiv), Pd(OAc)₂ (54 mg, 0.24 mmol, 0.4 equiv) and Cs₂CO₃ (587 mg, 1.80 mmol, 3.0 equiv) in portions at room temperature under nitrogen atmosphere. The resulting mixture was stirred for 1 h at 120° C. under nitrogen atmosphere. LCMS indicated the reaction was completed. The resulting mixture was filtered, the filter cake was washed with ACN and DCM. The filtrate was concentrated under reduced pressure. The residue was purified by silica gel column chromatography, eluted with CH₂Cl₂/MeOH (5:1) to afford 3-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]propanenitrile; formic acid (Compound 3, 220 mg, 53%) as a green solid. LCMS:(ES, m/s):609[M+H]⁺, 631 [M+Na]⁺.

### Step 3: Synthesis of Compound 5

To a stirred solution of 3-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]propanenitrile (200 mg, 0.32 mmol, 1.0 equiv) in THE (4.0 mL) was added triphosgene (146 mg, 0.49 mmol, 1.5 equiv) in portions at room temperature under nitrogen atmosphere. The resulting mixture was stirred for additional 30 min at room temperature. The resulting mixture was concentrated under vacuum. To the above mixture in DCM (2 mL) was added tert-butyl N-[(l S)-1-{[(1S)-1-{[4-(hydroxymethyl)phenyl]carbamoyl}ethyl]carbamoyl}-2-methylpropyl]carbamate (116 mg, 0.29 mmol, 0.9 equiv) in DCM (8.0 mL) and TEA (97 mg, 0.96 mmol, 3.0 equiv) at 0° C. The resulting mixture was stirred for additional 10 min at 0° C. Added DMAP (80 mg, 0.65 mmol, 2.0 equiv) in portions. The resulting mixture was stirred overnight at room temperature under nitrogen atmosphere. ˜10% desired product could be detected by LCMS. The resulting mixture was concentrated under vacuum. The residue was purified by reverse flash chromatography with the following conditions: column, C18 silica gel; mobile phase TFA, ACN in water, 0% to 50% gradient in 30 min; detector, UV 254 nm and 220 nm. The collected fraction was lyophilized to afford {4-[(2S)-2-[(2S)-2-[(tert-butoxycarbonyl)amino]-3-methylbutanamido]propanamido]phenyl}methyl N-(2-cyanoethyl)-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (Compound 5, 49 mg, 14%) as a green solid. LCMS: (ES, m/s): 514 [M/2+H]⁺, 1028 [M+H]⁺.

### Step 4: Synthesis of Compound 6

To a stirred solution of {4-[(2S)-2-[(2S)-2-[(tert-butoxycarbonyl)amino]-3-methylbutanamido]propanamido]phenyl}methyl N-(2-cyanoethyl)-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (Compound 5, 50 mg, 0.049 mmol, 1.0 equiv) in DCM (2.5 mL) were added TFA (0.5 mL) in portions at 0° C. under nitrogen atmosphere. The resulting mixture was stirred for 2 h at room temperature under nitrogen atmosphere. LCMS indicated the reaction was completed. The resulting mixture was concentrated under vacuum. The crude product was used in the next step directly without further purification. LCMS: (ES, m/s): 465 [M/2+H]⁺, 928 [M+H]⁺.

### Step 5: Synthesis of Compound 207

To a stirred solution of {4-[(2S)-2-[(2S)-2-amino-3-methylbutanamido]propanamido]phenyl}methyl N-(2-cyanoethyl)-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (Compound 6, 50 mg, 0.05 mmol, 1.0 equiv) in DMF (2.0 mL) were added DIEA (20 mg, 0.16 mmol, 3.0 equiv) and 2,5-dioxopyrrolidin-1-yl 6-(2,5-dioxopyrrol-1-yl)hexanoate (16 mg, 0.05 mmol, 1.0 equiv) in portions at 0° C. under nitrogen atmosphere. The resulting mixture was stirred for overnight at room temperature under nitrogen atmosphere. LCMS indicated the reaction was completed. The reaction mixture was purified by Prep-HPLC with the following conditions (Column: Mobile Phase A: Water (0.1% FA), Mobile Phase B: ACN; Flow rate: 25 mL/min). The collected fraction was lyophilized to afford formic acid; {4-[(2S)-2-[(2S)-2-[6-(2,5-dioxopyrrol-1-yl)hexanamido]-3-methylbutanamido]propanamido]phenyl}methyl N-(2-cyanoethyl)-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (8.6 mg, 13%) as a white solid. LCMS: (ES, m/s): 562 [M/2+H]⁺, 1122 [M+H]⁺; ¹H-NMR (300 MHz, DMSO-d₆) 9.99 (s, 1H), 8.41-8.18 (m, 3H), 8.10-7.94 (m, 3H), 7.83 (d, J=7.8 Hz, 1H), 7.62 (d, J=8.4 Hz, 2H), 7.50-7.25 (m, 3H), 6.99 (s, 2H), 5.28 (s, 2H), 5.16 (s, 2H), 4.94 (d, J=6 Hz, 2H), 4.77 (d, J=6.3 Hz, 2H), 4.41-4.34 (m, 1H), 4.20-4.14 (m, 3H), 3.65 (s, 2H), 3.50 (s, 2H), 3.21 (s, 3H), 2.96 (t, J=6.8 Hz, 2H), 2.71 (d, J=6 Hz, 2H), 2.23-2.06 (m, 2H), 1.99-1.90 (m, 2H), 1.66-1.58 (m, 4H), 1.49-1.45 (m, 5H), 1.30 (d, J=6.9 Hz, 3H), 1.23-1.15 (m, 2H), 0.95-0.60 (m, 12H).

### Synthesis of Compound 208

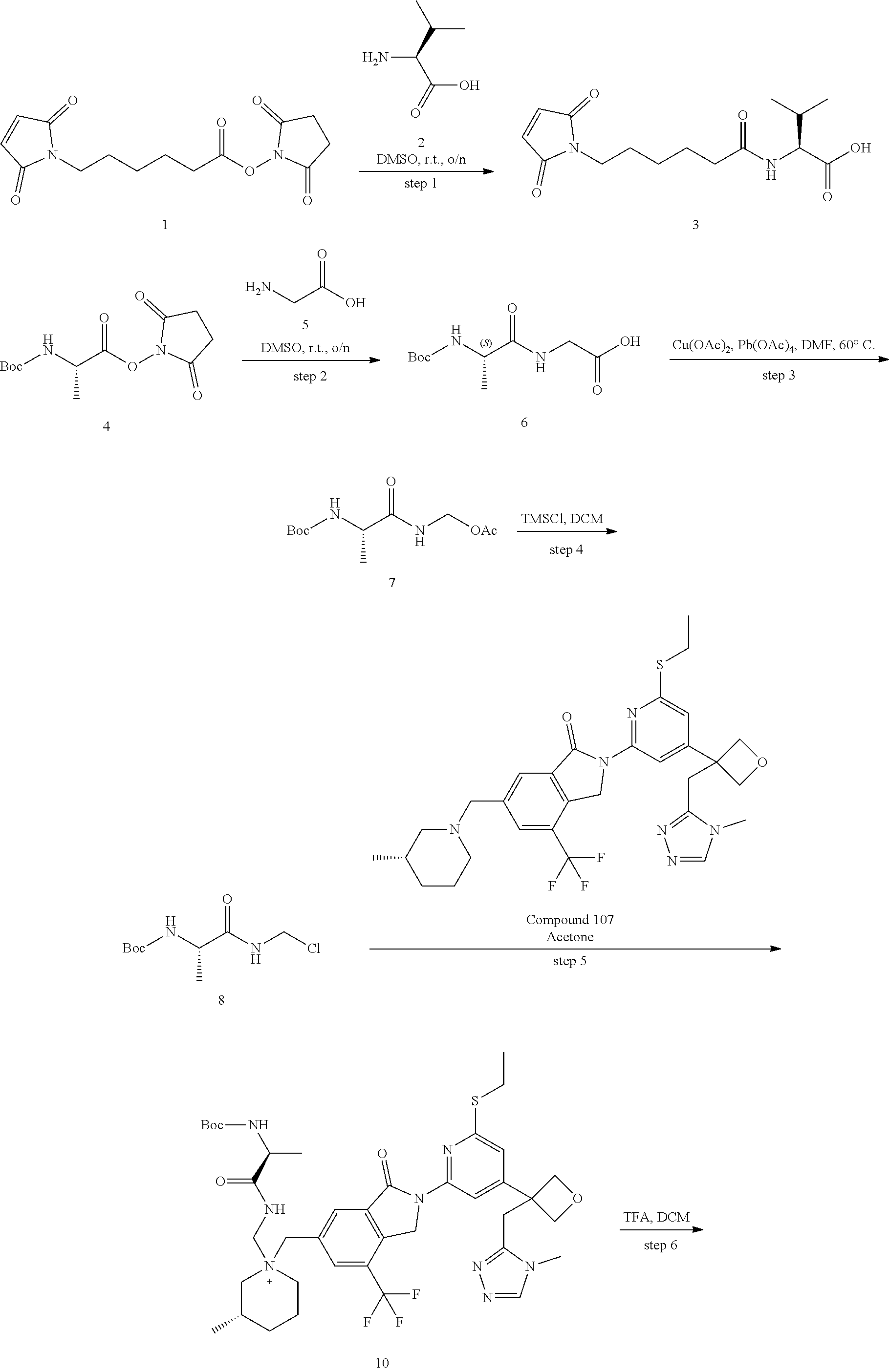

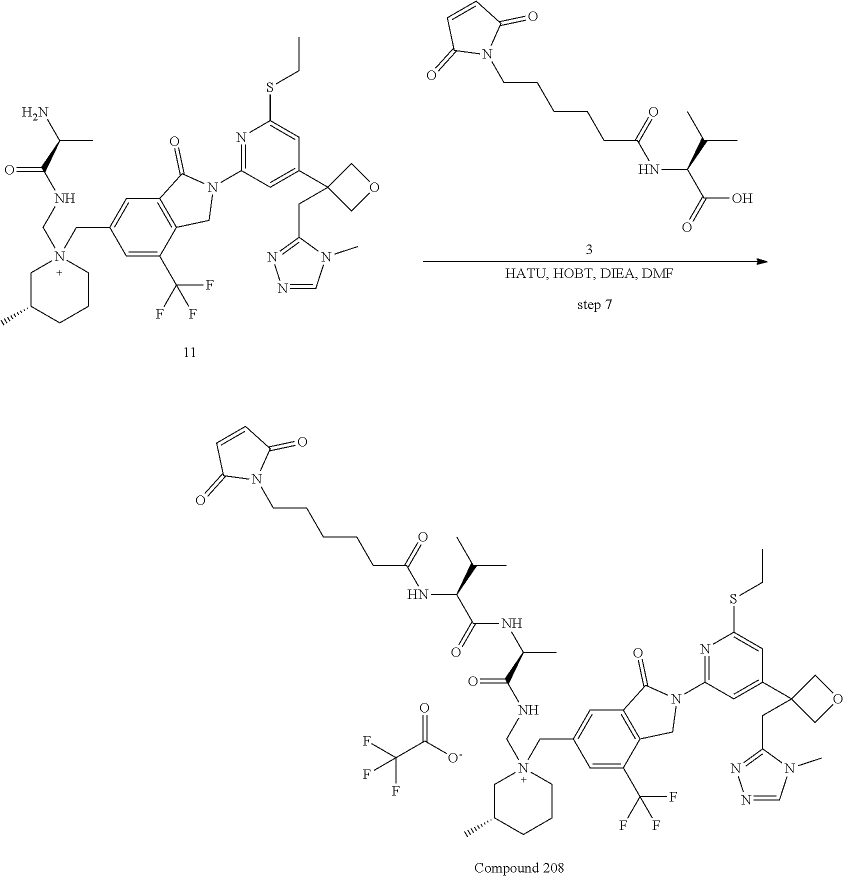

### Step 1: Synthesis of Compound 3

To a stirred solution of 2,5-dioxopyrrolidin-1-yl 6-(2,5-dioxopyrrol-1-yl)hexanoate (2 g, 6.48 mmol, 1 equiv) in DMSO (10 ml) was added L-valine (Compound 2, 0.84 g, 7.13 mmol, 1.1 equiv) in portions at room temperature. The resulting mixture was stirred for overnight at room temperature under air atmosphere. LCMS indicated the reaction 31% product and 40% the raw material. The reaction mixture was purified by reverse flash chromatography with the following conditions: column, silica gel; mobile phase, ACN in water (0.05% TFA), 0% to 50% gradient in 45 min; detector, UV 254 nm. This resulted in (2S)-2-[6-(2,5-dioxopyrrol-1-yl)hexanamido]-3-methylbutanoic acid (Compound 3, 600 mg, 290%) as a white solid. LCMS (ES, m/z): 311 [M+H]⁺

### Step 2: Synthesis of Compound 6

To a stirred solution of 2,5-dioxopyrrolidin-1-yl (2S)-2-[(tert-butoxycarbonyl)amino]propanoate (Compound 4, 10 g, 34.93 mmol, 1.0 equiv) in DMSO (100 mL) were added D-alanine (3.73 g, 41.91 mmol, 1.20 equiv) in portions at room temperature under nitrogen atmosphere. The resulting mixture was stirred for overnight at room temperature under nitrogen atmosphere. LCMS indicated the reaction was completed. The residue was purified by reverse flash chromatography with the following conditions: column, C18 silica gel; mobile phase, ACN in water (0.1% FA), 10% to 30% gradient in 30 min; detector, UV 220 nm and 200 nm. The resulting mixture was concentrated under vacuum to afford (2R)-2-[(2S)-2-[(tert-butoxycarbonyl)amino]propanamido]propanoic acid (Compound 6, 4.8 g, 47%) as a semi-solid. LCMS: (ES, m/z): 247 [M+H]⁺.

### Step 3: Synthesis of Compound 7

To a stirred solution of [(2S)-2-[(tert-butoxycarbonyl)amino]propanamido]acetic acid (Compound 6, 2.4 g, 9.74 mmol, 1 equiv) in DMF (25 ml) was added Cu(OAc)₂ (368 mg, 1.85 mmol, 0.2 equiv) and Pd(OAc)₄ (1350 mg, 4.15 mmol, 0.4 equiv) in portions at 0° C. under nitrogen atmosphere. The resulting mixture was stirred for overnight at 60° C. under nitrogen atmosphere. LCMS indicated the reaction was completed. The reaction was allowed to cool down to room temperature and quenched by the addition of water (70 mL) at 0° C. The aqueous layer was extracted with EtOAc (3×100 mL). The resulting mixture was concentrated under vacuum. The residue was purified by silica gel column chromatography, eluted with PE/EA (1:1) to afford [(2S)-2-[(tert-butoxycarbonyl)amino]propanamido]methyl acetate (Compound 7, 1.2 g, 47%) as a white solid. LCMS (ES, m/z): 261 [M+H]⁺

### Step 4: Synthesis of Compound 8

To a stirred solution of [(2S)-2-[(tert-butoxycarbonyl)amino]propanamido]methyl acetate (Compound 7, 200 mg, 0.76 mmol, 1 equiv) in DCM (2 mL) was added TMSCl (333 mg, 3.07 mmol, 4 equiv) dropwise at 0° C. under nitrogen atmosphere. The resulting mixture was stirred for 2 h at 0° C. under air atmosphere. Derivatization with MeOH for LCMS, LCMS indicated the reaction 25% product. The resulting mixture was concentrated under vacuum. This resulted in tert-butyl N-[(1S)-1-(chloromethylcarbamoyl)ethyl]carbamate (Compound 8, 150 mg, 82.47%) as a white solid. The resulting mixture was used in the next step directly without further purification. LCMS (ES, m/z):233 [M+H](Derivatization with MeOH)

### Step 5: Synthesis of Compound 10

The stirred solution of tert-butyl N-[(1S)-1-(chloromethylcarbamoyl)ethyl]carbamate (Compound 8, 78 mg, 0.33 mmol, 2 equiv) and 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-4-(trifluoromethyl)-3H-isoindol-1-one (Compound 107, 100 mg, 0.16 mmol, 1.00 equiv) in acetone (1 mL) was stirred for 2 h at 0° C. under nitrogen atmosphere. LCMS indicated the reaction was completed. The resulting mixture was concentrated under reduced pressure. The residue was purified by reverse flash chromatography with the following conditions: column, C18 silica gel; mobile phase, ACN in water (0.05% TFA), 0% to 60% gradient in 40 min; detector, UV 254 nm. The collected fraction as concentrated to afford (3S)-1-{[(2S)-2-[(tert-butoxycarbonyl)amino]propanamido]methyl}-1-({2-[6-(ethylsulfanyl)-4-{3-[(4-methyl-1,2,4-triazol-3-yl)methyl]oxetan-3-yl}pyridin-2-yl]-3-oxo-7-(trifluoromethyl)-1H-isoindol-5-yl}methyl)-3-methylpiperidin-1-ium (Compound 10, 90 mg, 67%) as a white solid. LCMS (ES, m/z): 801 [M+H]⁺

### Step 6: Synthesis of Compound 11

To a stirred solution of (3S)-1-{[(2S)-2-[(tert-butoxycarbonyl)amino]propanamido]methyl}-1-({2-[6-(ethylsulfanyl)-4-{3-[(4-methyl-1,2,4-triazol-3-yl)methyl]oxetan-3-yl}pyridin-2-yl]-3-oxo-7-(trifluoromethyl)-1H-isoindol-5-yl}methyl)-3-methylpiperidin-1-ium (Compound 10, 90 mg, 0.11 mmol, 1 equiv) in DCM (1 mL) was added TFA (200 uL) dropwise at 0° C. under air atmosphere. The resulting mixture was stirred for 1 h at 0° C. under nitrogen atmosphere. LCMS indicated the reaction was 30% product. The resulting mixture was concentrated under reduced pressure. The residue was purified by reverse flash chromatography with the following conditions: column, C18 silica gel; mobile phase, ACN in water (0.05% TFA), 0% to 50% gradient in 40 min; detector, UV 254 nm. This resulted in (3S)-1-{[(2S)-2-aminopropanamido]methyl}-1-({2-[6-(ethylsulfanyl)-4-{3-[(4-methyl-1,2,4-triazol-3-yl)methyl]oxetan-3-yl}pyridin-2-yl]-3-oxo-7-(trifluoromethyl)-1H-isoindol-5-yl}methyl)-3-methylpiperidin-1-ium (Compound 11, 50 mg, 63%) as a white oil. LCMS (ES, m/z): 701 [M+H]⁺

### Step 7: Synthesis of Compound 208

To a stirred solution of (2S)-2-[6-(2,5-dioxopyrrol-1-yl)hexanamido]-3-methylbutanoic acid (Compound 3, 53 mg, 0.17 mmol, 1.2 equiv) in DMF (1 mL) was added HATU (65 mg, 0.17 mmol, 1.2 equiv) in portions at 0° C. under air atmosphere. The resulting mixture was stirred for 10 min at 0° C. To the above mixture was added (3S)-1-{[(2S)-2-aminopropanamido]methyl}-1-({2-[6-(ethylsulfanyl)-4-{3-[(4-methyl-1,2,4-triazol-3-yl)methyl]oxetan-3-yl}pyridin-2-yl]-3-oxo-7-(trifluoromethyl)-1H-isoindol-5-yl}methyl)-3-methylpiperidin-1-ium (100 mg, 0.14 mmol, 1 equiv) and HOBT (19 mg, 0.14 mmol, 1 equiv) and DIEA (36 mg, 0.28 mmol, 2 equiv) dropwise at 0° C. The resulting mixture was stirred for additional 2 h at 0° C. LCMS indicated the reaction was completed. The reaction mixture was purified by reverse flash chromatography with the following conditions: column, C18 silica gel; mobile phase, ACN in water (0.05% TFA), 0% to 50% gradient in 40 min; detector, UV 254 nm. The collected fraction was concentrated to afford 30 mg crude product as a yellow solid. The crude product (30 mg) was purified by Prep-HPLC with the following conditions (Column: XSelect CSH Prep C18 OBD Column, 19*250 mm, 5 m; Mobile Phase A: Water (0.05% TFA), Mobile Phase B: ACN; Flow rate: 25 mL/min; Gradient: 25% B to 55% B in 7 min, 55% B; Wave Length: 254 nm; RT1 (min): 5; The collected fraction was lyophilized to afford to afford (3S)-1-{[(2S)-2-[(2S)-2-[6-(2,5-dioxopyrrol-1-yl)hexanamido]-3-methylbutanamido]propanamido]methyl}-1-({2-[6-(ethylsulfanyl)-4-{3-[(4-methyl-1,2,4-triazol-3-yl)methyl]oxetan-3-yl}pyridin-2-yl]-3-oxo-7-(trifluoromethyl)-1H-isoindol-5-yl}methyl)-3-methylpiperidin-1-ium (Compound 208, 13.5 mg, 9%) as a white solid. LCMS (ES, m/z): 993 [M+H−TFA]⁺; ¹H NMR (400 MHz, DMSO-d₆) δ 9.97-9.90 (m, 2H), 9.35 (t, J=15.2 Hz, 1H), 8.29-8.20 (m, 3H), 8.14 (d, J=6.4 Hz, 1H), 7.78 (d, J=8.0 Hz, 1H), 7.12-7.11 (m, 1H), 7.00-6.99 (m, 2H), 5.60-5.59 (m, 1H), 5.48-5.44 (m, 1H), 5.32 (s, 2H), 4.86-4.82 (m, 4H), 4.54 (br s, 2H), 4.21-4.09 (m, 2H), 3.85 (s, 2H), 3.72 (s, 3H), 3.38-3.34 (m, 3H), 3.30-3.27 (m, 1H), 3.20-3.15 (m, 2H), 2.93-2.80 (m, 1H), 2.69-2.58 (m, 1H) 2.16-2.08 (m, 2H), 1.90-1.74 (m, 5H), 1.49-1.43 (m, 4H), 1.39-1.35 (m, 3H), 1.18-1.12 (m, 5H), 1.10-1.04 (m, 1H), 0.90-0.88 (m, 3H), 0.82-0.78 (m, 6H).

### Synthesis of Compound 383

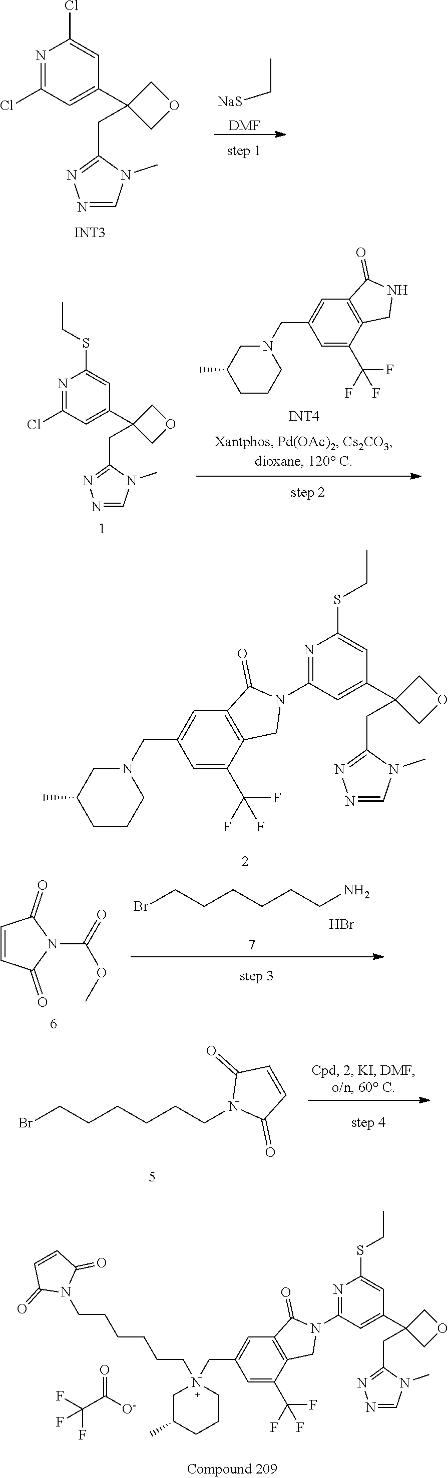

### Step 1: Synthesis of Compound 1

To a stirred solution of 2,6-dichloro-4-{3-[(4-methyl-1,2,4-triazol-3-yl)methyl]oxetan-3-yl}pyridine (INT3, 1 g, 3.34 mmol, 1 equiv) in DMF (11 mL) was added (ethylsulfanyl)sodium (0.34 g, 4.01 mmol, 1.2 equiv) dropwise at room temperature under nitrogen atmosphere. The resulting mixture was stirred for 2 h at 50° C. under nitrogen atmosphere. LCMS indicated the reaction was completed. The mixture was allowed to cool down to room temperature. The resulting mixture was concentrated under reduced pressure. The residue was purified by reverse flash chromatography with the following conditions: column, C18 silica gel; mobile phase, ACN in Water (0.05% TFA), 100% to 50% gradient in 30 min; detector, UV 254 nm. This resulted in 2-chloro-6-(ethylsulfanyl)-4-{3-[(4-methyl-1,2,4-triazol-3-yl)methyl]oxetan-3-yl}pyridine (Compound 1, 600 mg, 55%) as a white solid. LCMS (ES, m/z): 325,327 [M+H]0

### Step 2: Synthesis of Compound 2

To a stirred mixture of 2-chloro-6-(ethylsulfanyl)-4-{3-[(4-methyl-1,2,4-triazol-3-yl)methyl]oxetan-3-yl}pyridine (Compound 1, 300 mg, 0.92 mmol, 1 equiv) and 6-{[(3s)-3-methylpiperidin-1-yl]methyl}-4-(trifluoromethyl)-2,3-dihydroisoindol-1-one (INT4, 288 mg, 0.92 mmol, 1 equiv) in dioxane (9.3 mL) were added Xantphos (160 mg, 0.27 mmol, 0.3 equiv), Cs₂CO₃ (601 mg, 1.84 mmol, 2 equiv) and Pd(OAc)₂ (31 mg, 0.13 mmol, 0.15 equiv) dropwise at room temperature under nitrogen atmosphere. The resulting mixture was stirred for 2 h at 120° C. under nitrogen atmosphere. LCMS indicated the reaction was completed. The mixture was allowed to cool down to room temperature. The resulting mixture was filtered, the filter cake was washed with EtOAc (3×30 mL). The filtrate was concentrated under reduced pressure. The residue was purified by silica gel column chromatography, eluted with PE/EA (1:1) to afford 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-4-(trifluoromethyl)-3H-isoindol-1-one (230 mg, 41%) as a white solid. LCMS (ES, m/z): 601 [M+H]⁺.

### Step 3: Synthesis of Compound 5

To a stirred solution of 6-bromohexan-1-amine hydrobromide (Compound 7, 450 mg, 1.72 mmol, 1 equiv) in H₂O (7 mL) and saturated NaHCO₃ (aq.) (7 mL) was added methyl 2,5-dioxopyrrole-1-carboxylate (Compound 6, 320 mg, 2.06 mmol, 1.2 equiv) dropwise at 0° C. under nitrogen atmosphere. The resulting mixture was stirred for 40 min at 0° C. under nitrogen atmosphere. To the above mixture was added THE (7 mL) and saturated NaHCO₃ (aq.) (7 mL) in portions at 0° C. The resulting mixture was stirred for 40 min at 0° C. under nitrogen atmosphere. LCMS indicated the reaction was completed. The mixture was acidified to pH 6 with conc. HCl. The resulting mixture was extracted with EtOAc (3×50 mL). The combined organic layers were washed with brine (3×50 mL), dried over anhydrous Na₂SO₄. After filtration, the filtrate was concentrated under reduced pressure. This resulted in 1-(6-bromohexyl)pyrrole-2,5-dione (Compound 5, 360 mg, 80%) as a white solid. LCMS (ES, m/z): 260,262 [M+H]⁺

### Step 4: Synthesis of Compound 209

To a stirred mixture of 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-4-(trifluoromethyl)-3H-isoindol-1-one (Compound 2, 50 mg, 0.083 mmol, 1 equiv) and 1-(6-bromohexyl)pyrrole-2,5-dione (Compound 5, 108 mg, 0.41 mmol, 5 equiv) in DMF (3 mL) was added KI (13 mg, 0.083 mmol, 1 equiv) dropwise at room temperature under nitrogen atmosphere. The resulting mixture was stirred for overnight at 60° C. under nitrogen atmosphere. The mixture was allowed to cool down to room temperature. The reaction mixture was purified by reverse flash chromatography with the following conditions: column, C18 silica gel; mobile phase, ACN in Water (0.05% TFA), 10% to 50% gradient in 30 min; detector, UV 254 nm. The collected fraction was concentrated to dryness under vacuum. The crude product was re-purified by the following condition: Column: XSelect CSH Prep C18 OBD Column, 19×250 mm, S m; Mobile Phase A: Water (0.05% TFA), Mobile Phase B: ACN; Flow rate: 25 mL/min; Gradient: 27% B to 57% B in 7 min, 57% B; Wave Length: 254 nm; RT1 (min): 6; The collected fraction was lyophilized to afford (3S)-1-[6-(2,5-dioxopyrrol-1-yl)hexyl]-1-({2-[6-(ethylsulfanyl)-4-{3-[(4-methyl-1,2,4-triazol-3-yl)methyl]oxetan-3-yl}pyridin-2-yl]-3-oxo-7-(trifluoromethyl)-1H-isoindol-5-yl}methyl)-3-methylpiperidin-1-ium (Compound 209, 4.9 mg, 5%) as a white solid. LCMS (ES, m/z): 781 [M+H−TFA]⁺; ¹H-NMR (DMSO-d₆, 400 MHz) δ (ppm): 9.87 (s, 1H), 8.27-8.21 (m, 3H), 7.10-6.98 (m, 3H), 5.32 (s, 2H), 4.88-4.84 (m, 4H), 4.54-4.52 (m, 2H), 4.25-4.23 (m, 2H), 3.85-3.83 (m, 2H), 3.72 (s, 3H), 3.32-3.20 (m, 5H), 2.90-2.85 (m, 1H), 1.90-1.65 (m, 6H), 1.36-1.25 (m, 6H), 1.20-1.00 (m, 5H), 0.89-0.88 (m, 4H).

### Synthesis of Compound 210

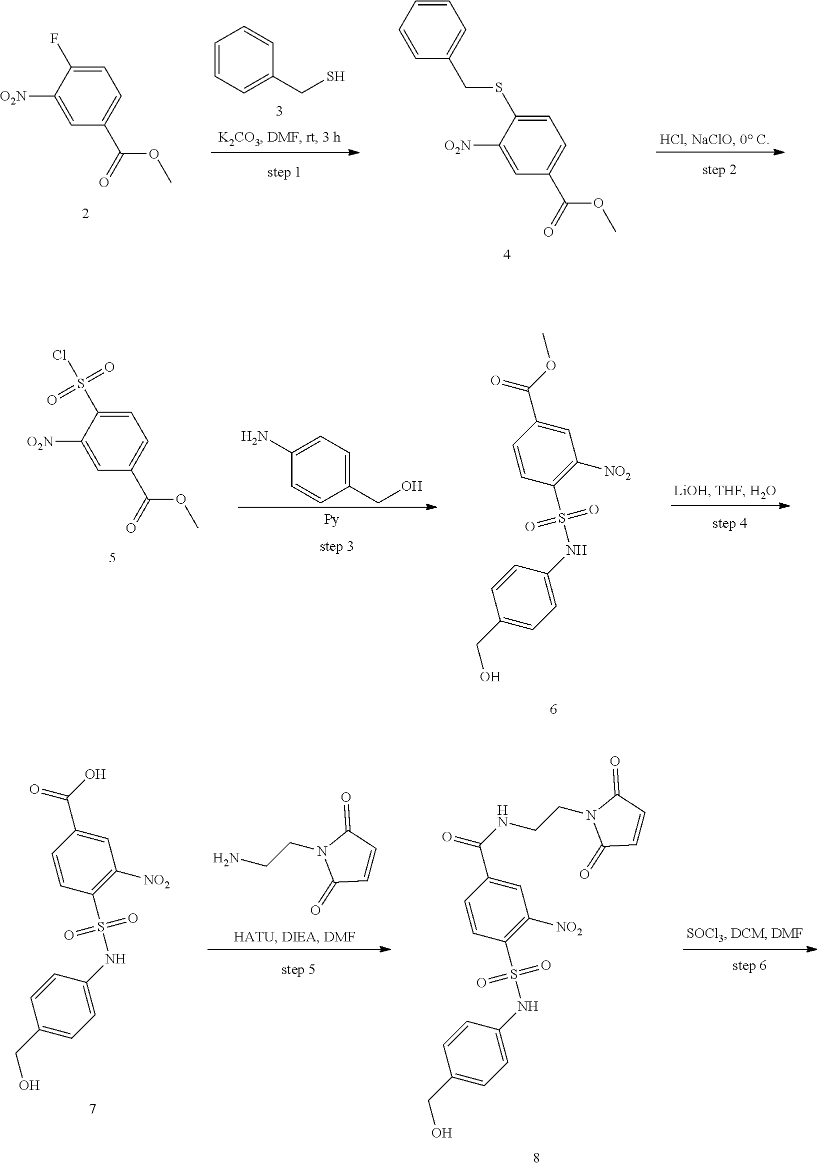

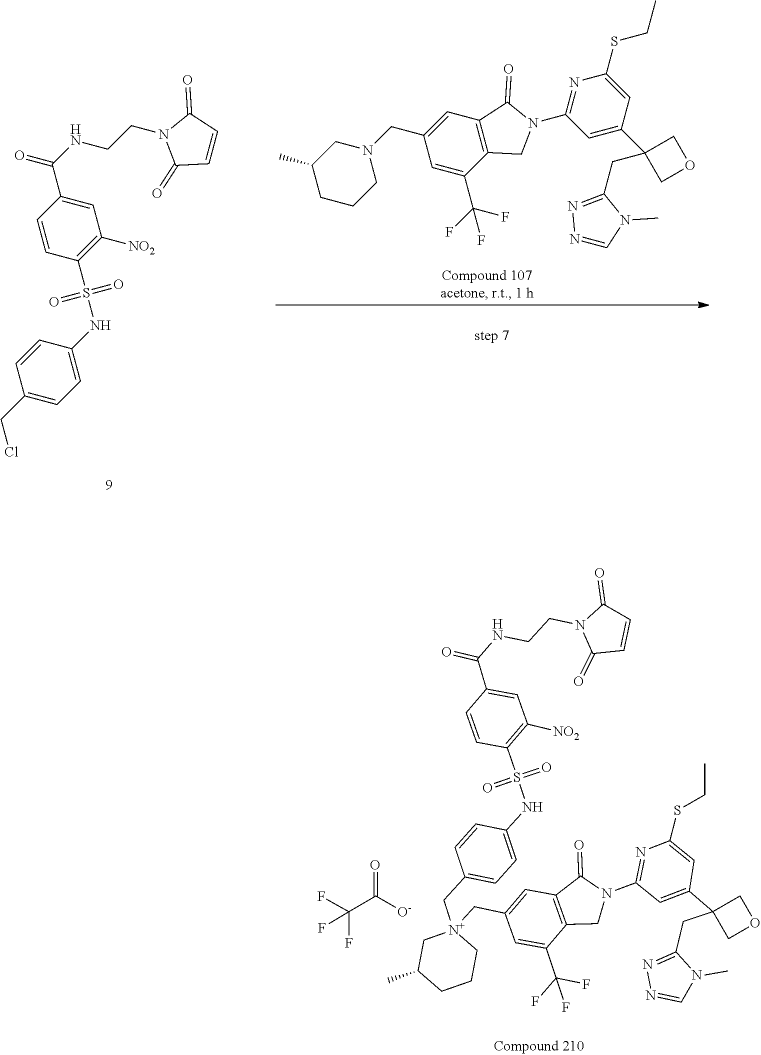 [SMILES: O=C(NCCN1C(=O)C=CC1=O)c1ccc(S(=O)(=O)Nc2ccc(CCl)cc2)c([N+](=O)[O-])c1 ; CCSc1cc(C2(Cc3nncn3C)COC2)cc(N2Cc3c(cc(CN4CCC[C@H](C)C4)cc3C(F)(F)F)C2=O)n1 ; O=C([O-])C(F)(F)F ; CCSc1cc(C2(Cc3nncn3C)COC2)cc(N2Cc3c(cc(C[N+]4(Cc5ccc(NS(=O)(=O)c6ccc(C(=O)NCCN7C(=O)C=CC7=O)cc6[N+](=O)[O-])cc5)CCC[C@H](C)C4)cc3C(F)(F)F)C2=O)n1]

### Step 1: Synthesis of Compound 4

To a stirred mixture of methyl 4-fluoro-3-nitrobenzoate (Compound 2, 10 g, 50.21 mmol, 1 equiv) and K₂CO₃ (13.88 g, 100.43 mmol, 2 equiv) in DMF (160 mL) was added benzyl mercaptan (Compound 3, 12.47 g, 100.43 mmol, 2 equiv) dropwise at 0° C. The resulting mixture was stirred for 3 h at 25° C. LCMS indicated the reaction was completed. The reaction was quenched by the addition of Water (450 mL) at 0° C. The precipitated solids were collected by filtration and washed with water (3×150 mL). The solid was purified by trituration with PE (300 mL). This resulted in methyl 4-(benzylsulfanyl)-3-nitrobenzoate (Compound 4, 10 g, 65%) as a yellow solid. LCMS (ES, m/z): 304 [M+H]⁺

### Step 2: Synthesis of Compound 5

To a stirred mixture of methyl 4-(benzylsulfanyl)-3-nitrobenzoate (Compound 4, 10 g, 36.32 mmol, 1 equiv) in DCM (200 mL) was added HCl (6N, 200 mL) and NaClO (100 mL) dropwise at 0° C. The resulting mixture was stirred for 1 h at 0° C. LCMS indicated the reaction was completed. The resulting mixture was extracted with CH₂Cl₂ (3×20 mL). The combined organic layers were washed with brine (300 mL), dried over anhydrous Na₂SO₄. After filtration, the filtrate was concentrated under reduced pressure. The residue was purified by silica gel column chromatography, eluted with PE/EA (1:4) to afford methyl 4-(chlorosulfonyl)-3-nitrobenzoate (Compound 5, 8 g, 78%) as a yellow solid. ¹H NMR (300 MHz, CDCl₃) δ 8.51-8.46 (m, 2H), 8.38 (d, J=8.1 Hz, 1H), 4.06 (s, 3H).

### Step 3: Synthesis of Compound 6

To a stirred solution of methyl 4-(chlorosulfonyl)-3-nitrobenzoate (Compound 5, 1.2 g, 4.29 mmol, 1 equiv) in pyridine (12 mL) was added p-aminobenzylalcohol (0.63 g, 5.14 mmol, 1.2 equiv) in portions at 0° C. under air atmosphere. The resulting mixture was stirred for 30 min at 0° C. under air atmosphere. 50% desired product was found by LCMS. The mixture was acidified to pH 3 with 1N HCl. The precipitated solids were collected by filtration and washed with Et₂O (3×50 mL). The solid was purified by silica gel column chromatography, eluted with CH₂Cl₂/MeOH (10:1) to afford methyl 4-{[4-(hydroxymethyl)phenyl]sulfamoyl}-3-nitrobenzoate (Compound 6, 850 mg, 49%) as a yellow solid. LCMS:(ES·m/z):349[M+H−H₂O]⁺.

### Step 4: Synthesis of Compound 7

To a stirred solution of methyl 4-{[4-(hydroxymethyl)phenyl]sulfamoyl}-3-nitrobenzoate (Compound 6, 850 mg, 2.32 mmol, 1 equiv) in THE (8 mL) was added LiGH (222 mg, 9.28 mmol, 4 equiv) in H₂O (4 mL) dropwise at 0° C. The resulting mixture was stirred for 2 h at 0° C. under air atmosphere. LCMS indicated the reaction was completed. The reaction mixture was acidified to pH 3 with 1N HCl. The precipitated solids were collected by filtration and washed with Et₂O (3×50 mL). This resulted in 4-{[4-(hydroxymethyl)phenyl]sulfamoyl}-3-nitrobenzoic acid (Compound 7, 270 mg, 29%) as a yellow solid. LCMS:(ES·m/z):335[M+H−H₂O]⁺.

### Step 5: Synthesis of Compound 8

To a stirred mixture of 4-{[4-(hydroxymethyl)phenyl]sulfamoyl}-3-nitrobenzoic acid (Compound 7, 730 mg, 2.07 mmol, 0.97 equiv) and HATU (1220 mg, 3.21 mmol, 1.50 equiv) in DMF (20 mL) was stirred for 15 min at 0° C. To the above mixture was added 1-(2-aminoethyl)pyrrole-2,5-dione (300 mg, 2.14 mmol, 1.00 equiv) in portions and DIEA (1106 mg, 8.56 mmol, 4.00 equiv) dropwise at 0° C. The resulting mixture was stirred for additional 2 h at 0° C. 30% desired product was found by LCMS. The reaction was quenched with water at 0° C. The resulting mixture was extracted with EtOAc (3×80 mL). The combined organic layers were washed with brine (2×30 mL), dried over anhydrous Na₂SO₄. After filtration, the filtrate was concentrated under reduced pressure. The residue was purified by silica gel column chromatography, eluted with CH₂Cl₂/MeOH (10:1) to afford N-[2-(2,5-dioxopyrrol-1-yl)ethyl]-4-{[4-(hydroxymethyl)phenyl]sulfamoyl}-3-nitrobenzamide (Compound 8, 190 mg, 16%) as a yellow solid. LCMS:(ES·m/z):457[M+H−H₂O]⁺.

### Step 6: Synthesis of Compound 9

To a stirred solution of N-[2-(2,5-dioxopyrrol-1-yl)ethyl]-4-{[4-(hydroxymethyl)phenyl]sulfamoyl}-3-nitrobenzamide (25 mg, 0.053 mmol, 1 equiv) and in DCM (4 mL) was added SOCl₂ (18 mg, 0.16 mmol, 3 equiv) dropwise at 0° C. under air atmosphere. The resulting mixture was stirred for 2 h at 0° C. under air atmosphere. LCMS indicated the reaction was completed. The resulting mixture was concentrated under reduced pressure. The crude product was used in the next step directly without further purification. LCMS:(ES·m/z):493[M+H]⁺.

### Step 9: Synthesis of Compound 210

To a stirred mixture of 4-{[4-(chloromethyl)phenyl]sulfamoyl}-N-[2-(2,5-dioxopyrrol-1-yl)ethyl]-3-nitrobenzamide (40 mg, 0.081 mmol, 0.81 equiv) and 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-4-(trifluoromethyl)-3H-isoindol-1-one (Compound 107, 60 mg, 0.10 mmol, 1.00 equiv) in acetone (5 mL) at room temperature under air atmosphere. The resulting mixture was stirred for 1 h at 0° C. under air atmosphere. LCMS indicated the reaction was completed. The reaction mixture was purified by reverse flash chromatography with the following conditions: column, C18 silica gel; mobile phase, MeCN in water (0.1% TFA), 10% to 50% gradient in 30 min; detector, UV 254 nm. The crude product was re-purified by Prep-HPLC with the following conditions (Column: XBridge Prep OBD C18 Column, 19*250 mm, 5 m; Mobile Phase A: Water (0.05% TFA), Mobile Phase B: ACN; Flow rate: 25 mL/min; Gradient: 32% B to 52% B in 11 min, 52% B; Wave Length: 254 nm; RT1 (min): 10.15; The collected fraction was lyophilized to afford (3S)-1-{[4-(4-{[2-(2,5-dioxopyrrol-1-yl)ethyl]carbamoyl}-2-nitrobenzenesulfonamido)phenyl]methyl}-1-({2-[6-(ethylsulfanyl)-4-{3-[(4-methyl-1,2,4-triazol-3-yl)methyl]oxetan-3-yl}pyridin-2-yl]-3-oxo-7-(trifluoromethyl)-1H-isoindol-5-yl}methyl)-3-methylpiperidin-1-ium trifluoroacetate (Compound 210, 7 mg, 5%) as a white solid. LCMS:(ms, ESI):1057[M−TFA]⁺; ¹H NMR (400 MHz, DMSO-d₆) δ 11.10 (s, 1H), 10.25 (br s, 1H), 9.01 (t, J=5.4 Hz, 1H), 8.27 (d, J=6.8 Hz, 2H), 8.18 (s, 2H), 8.05 (s, 2H), 7.30-7.26 (m, 2H), 7.17-7.05 (m, 3H), 6.99 (s, 2H), 5.43 (s, 2H), 5.32 (s, 2H), 4.84-4.80 (m, 4H), 4.70 (s, 2H), 3.81 (s, 2H), 3.66 (s, 3H), 3.64-3.63 (m, 2H), 3.39-3.38 (m, 2H), 3.37-3.36 (m, 1H), 3.18-3.16 (m, 2H), 2.85-2.83 (m, 1H), 2.60-2.58 (m, 1H), 1.85-1.65 (m, 4H), 1.38-1.35 (m, 3H), 1.08-1.05 (m, 1H), 0.88 (d, J=6.4 Hz, 3H).

### Synthesis of Compound 211

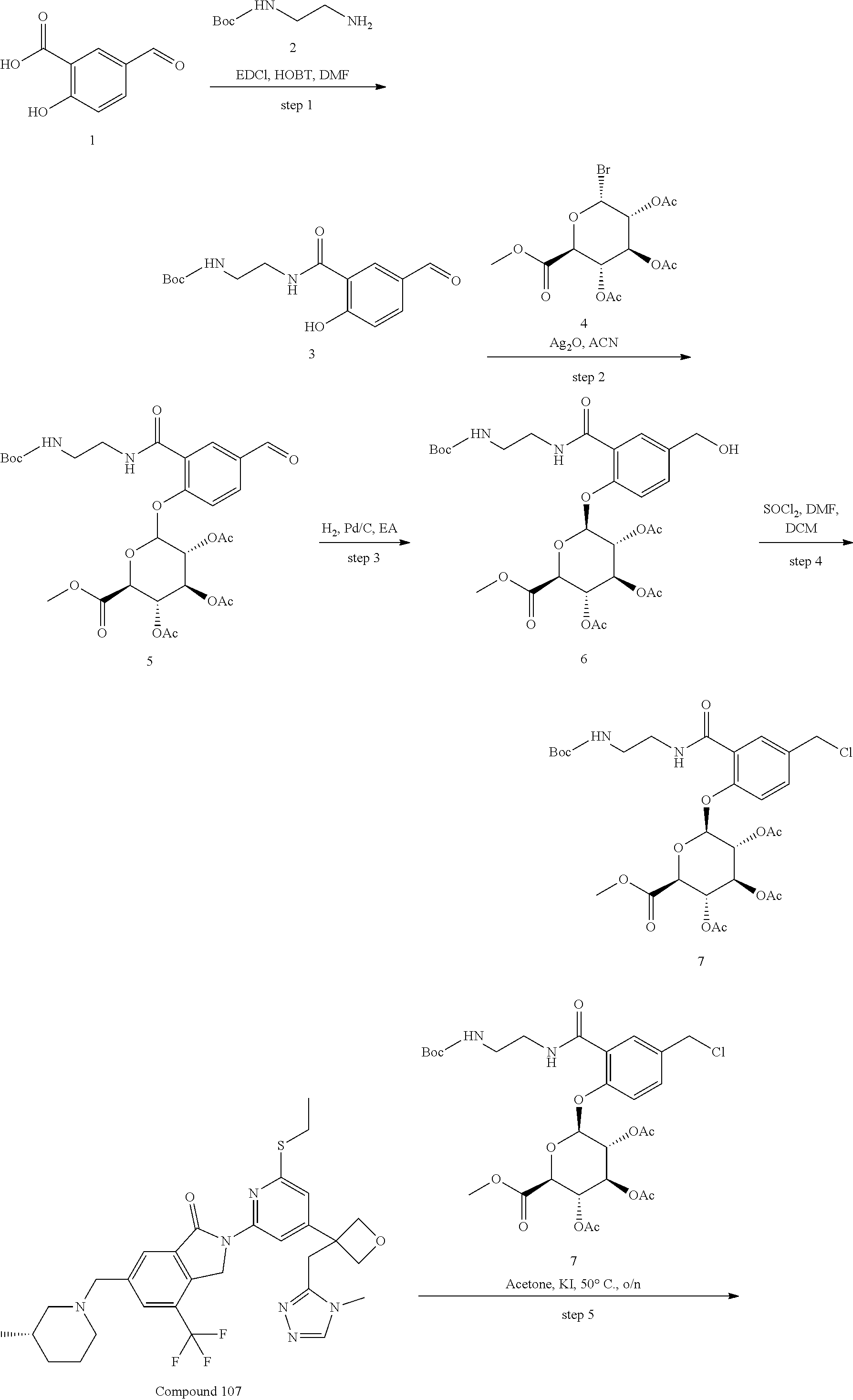

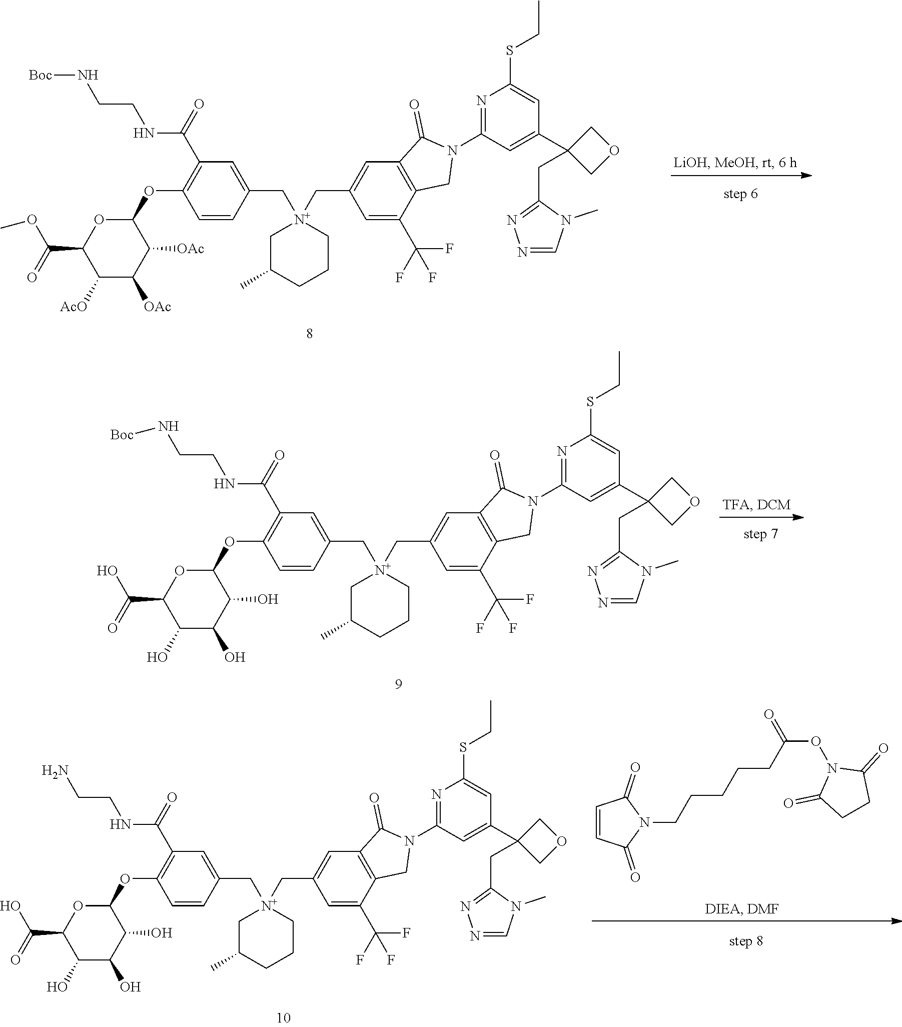

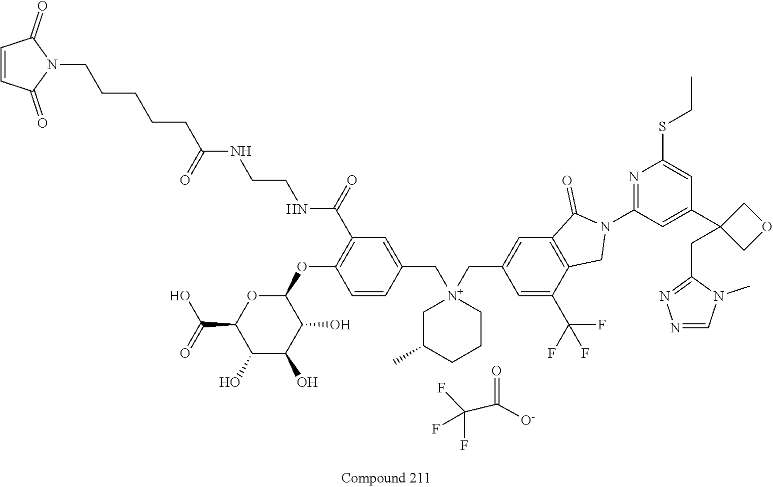 [SMILES: CCSc1cc(C2(Cc3nncn3C)COC2)cc(N2Cc3c(cc(C[N+]4(Cc5ccc(O[C@@H]6O[C@H](C(=O)O)[C@@H](O)[C@H](O)[C@H]6O)c(C(=O)NCCNC(=O)CCCCCN6C(=O)C=CC6=O)c5)CCC[C@H](C)C4)cc3C(F)(F)F)C2=O)n1 ; O=C([O-])C(F)(F)F]

### Step 1: Synthesis of Compound 3

To a stirred solution of 5-formyl-2-hydroxybenzoic acid (Compound 1, 20 g, 120.38 mmol, 1.00 equiv) in DMF (200 mL) were added EDCI (28 g, 144.44 mmol, 1.20 equiv), HOBT (20 g, 144.46 mmol, 1.20 equiv) and tert-butyl N-(2-aminoethyl)carbamate (Compound 2, 23 g, 144.46 mmol, 1.20 equiv) in portions at 0° C. under nitrogen atmosphere. The resulting mixture was stirred for overnight at room temperature under nitrogen atmosphere. LCMS indicated the reaction was completed. The reaction mixture was quenched with water and extracted with EA(3×200 mL). The combined organic was washed with brine (200 mL), dried with Na₂SO₄. After filtration, the filtrate was concentrated to dryness under vacuum. The residue was purified by silica gel column chromatography, eluted with PE/EA (2:3) to afford tert-butyl N-[2-[(5-formyl-2-hydroxyphenyl)formamido]ethyl]carbamate (Compound 3, 23 g, 53%) as a white solid. LCMS (ES, m/z): 209 [M+H−100]⁺, 309 [M+H]⁺, 331 [M+Na]⁺.

### Step 2: Synthesis of Compound 5

To a stirred solution of tert-butyl N-[2-[(5-formyl-2-hydroxyphenyl)formamido]ethyl]carbamate (Compound 3, 23 g, 74.59 mmol, 1.00 equiv) in ACN (600 mL) were added Ag₂O (34.57 g, 149.17 mmol, 2.00 equiv) and methyl (2S,3S,4S,5R,6R)-3,4,5-tris(acetyloxy)-6-bromooxane-2-carboxylate (Compound 4, 32.6 g, 82.05 mmol, 1.10 equiv) in portions at 0° C. under nitrogen atmosphere. The resulting mixture was stirred for overnight at room temperature under nitrogen atmosphere. LCMS indicated the reaction was completed. After filtration, the filtrate was concentrated to dryness under vacuum. The residue was purified by silica gel column chromatography, eluted with PE/EA (1:4) to afford methyl (2S,3S,4S,5R,6S)-3,4,5-tris(acetyloxy)-6-[2-([2-[(tert-butoxycarbonyl)amino]ethyl]carbamoyl)-4-formylphenoxy]oxane-2-carboxylate (Compound 5, 38 g, 76%) as a green solid. LCMS (ES, m/z): 525 [M+H−100]⁺, 625 [M+H]⁺, 647 [M+Na]⁺.

### Step 3: Synthesis of Compound 6

A mixture of methyl (2S,3S,4S,5R,6S)-3,4,5-tris(acetyloxy)-6-[2-({2-[(tert-butoxycarbonyl)amino]ethyl}carbamoyl)-4-formylphenoxy]oxane-2-carboxylate (Compound 5, 500 mg, 0.80 mmol, 1 equiv) and Pd/C (200 mg, 10%) in EA (15 mL) was stirred for 1 h at 25° C. under hydrogen atmosphere. LCMS indicated the reaction was completed. The resulting mixture was filtered and the filter cake was washed with EA (15 mL). The filtrate was concentrated under reduced pressure to afford methyl (2S,3S,4S,5R,6S)-3,4,5-tris(acetyloxy)-6-[2-({2-[(tert-butoxycarbonyl)amino]ethyl}carbamoyl)-4-(hydroxymethyl)phenoxy]oxane-2-carboxylate (Compound 6, 480 mg, 95%) as a white solid. LCMS (ESI, m/z): 627 [M+H]⁺.

### Step 4: Synthesis of Compound 7

A mixture of methyl (2S,3S,4S,5R,6S)-3,4,5-tris(acetyloxy)-6-[2-({2-[(tert-butoxycarbonyl)amino]ethyl}carbamoyl)-4-(hydroxymethyl)phenoxy]oxane-2-carboxylate (Compound 5, 5 g, 7.97 mmol, 1 equiv) and DMF (0.3 g, 4.10 mmol, 0.51 equiv) in DCM (60 mL) was added thionyl chloride (1.90 g, 15.95 mmol, 2 equiv) dropwise at 0° C. The resulting mixture was stirred for 2 h at 0° C. LCMS indicated the reaction was completed. The reaction was quenched by addition of water (10 mL) at 0° C., then washed with saturated sodium bicarbonate solution (30 mL). The organic layer was dried over sodium sulfate and concentrated. The residue was purified by silica gel column chromatography, eluted with DCM/MeOH (96:4) to afford methyl (2S,3S,4S,5R,6S)-3,4,5-tris(acetyloxy)-6-[2-({2-[(tert-butoxycarbonyl)amino]ethyl}carbamoyl)-4-(chloromethyl)phenoxy]oxane-2-carboxylate (Compound 7, 3 g, 58%) as a white solid. LCMS (ESI, m/z): 627 [M+H]⁺.

### Step 5: Synthesis of Compound 8

A mixture of 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-4-(trifluoromethyl)-3H-isoindol-1-one (Compound 107, 250 mg, 0.41 mmol, 1 equiv), KI (138 mg, 0.83 mmol, 2 equiv) and methyl (2S,3S,4S,5R,6S)-3,4,5-tris(acetyloxy)-6-[2-({2-[(tert-butoxycarbonyl)amino]ethyl}carbamoyl)-4-(chloromethyl)phenoxy]oxane-2-carboxylate (Compound 7, 332 mg, 0.51 mmol, 1.24 equiv) in acetone (10 mL) was stirred for 6 h at 50° C. LCMS indicated the reaction was completed. The solvent was removed under reduced pressure. The residue was purified by reverse phase flash with the following conditions: column, C18 silica gel, 80 g, 20-35 um; mobile phase, water with 0.1% TFA and ACN (0% to 100% gradient in 50 min; detector, UV 254 nm. The eluent was concentrated to afford (3S)-1-{[3-({2-[(tert-butoxycarbonyl)amino]ethyl}carbamoyl)-4-{[(2S,3R,4S,5S,6S)-3,4,5-tris(acetyloxy)-6-(methoxycarbonyl)oxan-2-yl]oxy}phenyl]methyl}-1-({2-[6-(ethylsulfanyl)-4-{3-[(4-methyl-1,2,4-triazol-3-yl)methyl]oxetan-3-yl}pyridin-2-yl]-3-oxo-7-(trifluoromethyl)-1H-isoindol-5-yl}methyl)-3-methylpiperidin-1-ium (Compound 8, 200 mg, 39%) as a white solid. LCMS (ES, m/z): 1209 [M]⁺.

### Step 6: Synthesis of Compound 9

A mixture of (3S)-1-{[3-({2-[(tert-butoxycarbonyl)amino]ethyl}carbamoyl)-4-{[(2S,3R,4S,5S,6S)-3,4,5-tris(acetyloxy)-6-(methoxycarbonyl)oxan-2-yl]oxy}phenyl]methyl}-1-({2-[6-(ethylsulfanyl)-4-{3-[(4-methyl-1,2,4-triazol-3-yl)methyl]oxetan-3-yl}pyridin-2-yl]-3-oxo-7-(trifluoromethyl)-1H-isoindol-5-yl}methyl)-3-methylpiperidin-1-ium (Compound 8, 200 mg, 0.16 mmol, 1 equiv) and LiOH (40 mg, 1.66 mmol, 10.11 equiv) in MeOH (4 mL) was stirred for 2 h at 25° C. LCMS indicated the reaction was completed. The solvent was removed with nitrogen flow to afford crude (3S)-1-{[3-({2-[(tert-butoxycarbonyl)amino]ethyl}carbamoyl)-4-{[(2S,3R,4S,5S,6S)-6-carboxy-3,4,5-trihydroxyoxan-2-yl]oxy}phenyl]methyl}-1-({2-[6-(ethylsulfanyl)-4-{3-[(4-methyl-1,2,4-triazol-3-yl)methyl]oxetan-3-yl}pyridin-2-yl]-3-oxo-7-(trifluoromethyl)-1H-isoindol-5-yl}methyl)-3-methylpiperidin-1-ium (Compound 9, 260 mg, crude) as a yellow solid. LCMS (ESI, m/z): 1069 [M]⁺.

### Step 7: Synthesis of Compound 10

A mixture of (3S)-1-{[3-({2-[(tert-butoxycarbonyl)amino]ethyl}carbamoyl)-4-{[(2S,3R,4S,5S,6S)-6-carboxy-3,4,5-trihydroxyoxan-2-yl]oxy}phenyl]methyl}-1-({2-[6-(ethylsulfanyl)-4-{3-[(4-methyl-1,2,4-triazol-3-yl)methyl]oxetan-3-yl}pyridin-2-yl]-3-oxo-7-(trifluoromethyl)-1H-isoindol-5-yl}methyl)-3-methylpiperidin-1-ium (Compound 9, 150 mg, 0.14 mmol, 1 equiv) and TFA (1.5 mL) in DCM (7.5 mL) was stirred for 1 h at 25° C. LCMS indicated the reaction was completed. The solvent was removed with nitrogen flow. The residue was dissolved with water (2 mL) and washed with DCM (3×3 mL). The water phase was dried by lyophilization to afford (3S)-1-({3-[(2-aminoethyl)carbamoyl]-4-{[(2S,3R,4S,5S,6S)-6-carboxy-3,4,5-trihydroxyoxan-2-yl]oxy}phenyl}methyl)-1-({2-[6-(ethylsulfanyl)-4-{3-[(4-methyl-1,2,4-triazol-3-yl)methyl]oxetan-3-yl}pyridin-2-yl]-3-oxo-7-(trifluoromethyl)-1H-isoindol-5-yl}methyl)-3-methylpiperidin-1-ium (Compound 10, 140 mg, 87% for two steps) as a brown solid. LCMS (ESI, m/z): 969 [M]f.

### Step 8: Synthesis of Compound 211

A mixture of (3S)-1-({3-[(2-aminoethyl)carbamoyl]-4-{[(2S,3R,4S,5S,6S)-6-carboxy-3,4,5-trihydroxyoxan-2-yl]oxy}phenyl}methyl)-1-({2-[6-(ethylsulfanyl)-4-{3-[(4-methyl-1,2,4-triazol-3-yl)methyl]oxetan-3-yl}pyridin-2-yl]-3-oxo-7-(trifluoromethyl)-1H-isoindol-5-yl}methyl)-3-methylpiperidin-1-ium (Compound 10, 120 mg, 0.124 mmol, 1 equiv), 2,5-dioxopyrrolidin-1-yl 6-(2,5-dioxopyrrol-1-yl)hexanoate (38 mg, 0.12 mmol, 1 equiv) and DIEA (95 mg, 0.74 mmol, 6 equiv) in DMF (3 mL) was stirred for 1 h at 25° C. LCMS indicated the reaction was completed. The reaction was purified by prep-HPLC with the following condition: Column: Sunfire Prep C18 OBD Column, 19*250 mm, 10 m; Mobile Phase A: Water (0.05% TFA), Mobile Phase B: ACN; Flow rate: 25 mL/min; Gradient: 20% B to 27% B in 12 min, 27% B to 27% B in 20 min, 27% B; Wave Length: 254 nm; RT1 (min): 16.3; The collected fraction was lyophilized to afford (3S)-1-[(4-{[(2S,3R,4S,5S,6S)-6-carboxy-3,4,5-trihydroxyoxan-2-yl]oxy}-3-({2-[6-(2,5-dioxopyrrol-1-yl)hexanamido]ethyl}carbamoyl)phenyl)methyl]-1-({2-[6-(ethylsulfanyl)-4-{3-[(4-methyl-1,2,4-triazol-3-yl)methyl]oxetan-3-yl}pyridin-2-yl]-3-oxo-7-(trifluoromethyl)-1H-isoindol-5-yl}methyl)-3-methylpiperidin-1-ium (Compound 211, 8.2 mg, 5%) as a white solid. LCMS (ES, m/z): 1162 [M−TFA]*; ¹H NMR (400 MHz, Methanol-d4) δ 8.34 (s, 1H), 8.17 (s, 1H), 7.96 (s, 1H), 7.89 (d, J=2.0 Hz, 1H), 7.62-7.56 (m, 2H), 7.05 (s, 1H), 6.80 (s, 2H), 5.59-5.48 (m, 2H), 5.43-5.30 (m, 2H), 5.12-5.03 (m, 2H), 4.95-4.92 (m, 2H), 4.85-4.82 (m, 1H), 4.63-4.52 (m, 2H), 4.04-3.85 (m, 3H), 3.67-3.36 (m, 12H), 3.25-3.21 (m, 3H), 2.96-2.94 (s, 1H), 2.74-2.67 (m, 1H), 2.19-1.80 (m, 7H), 1.65-1.50 (m, 4H), 1.48-1.41 (m, 3H), 1.32-1.18 (m, 3H), 1.00 (d, J=6.4 Hz, 3H).

### Synthesis of Compound 212

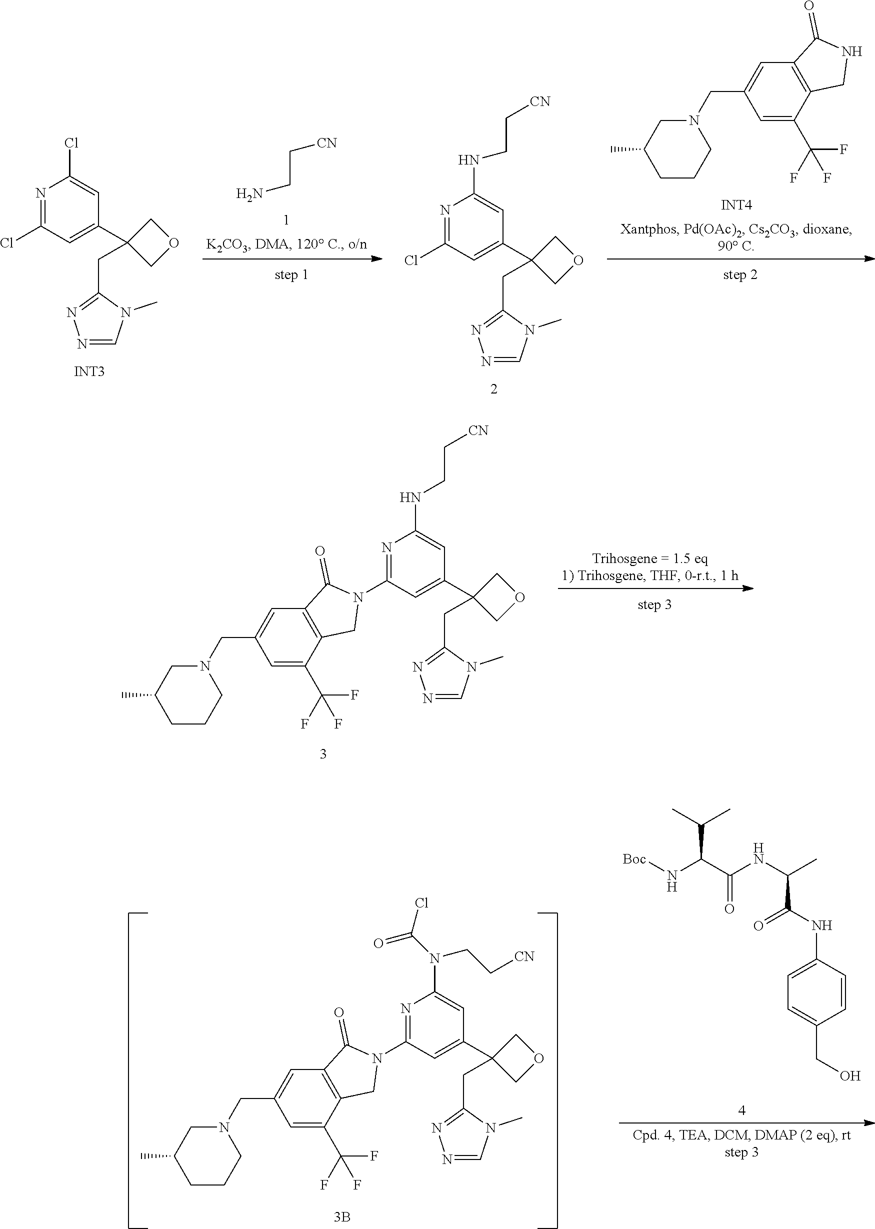

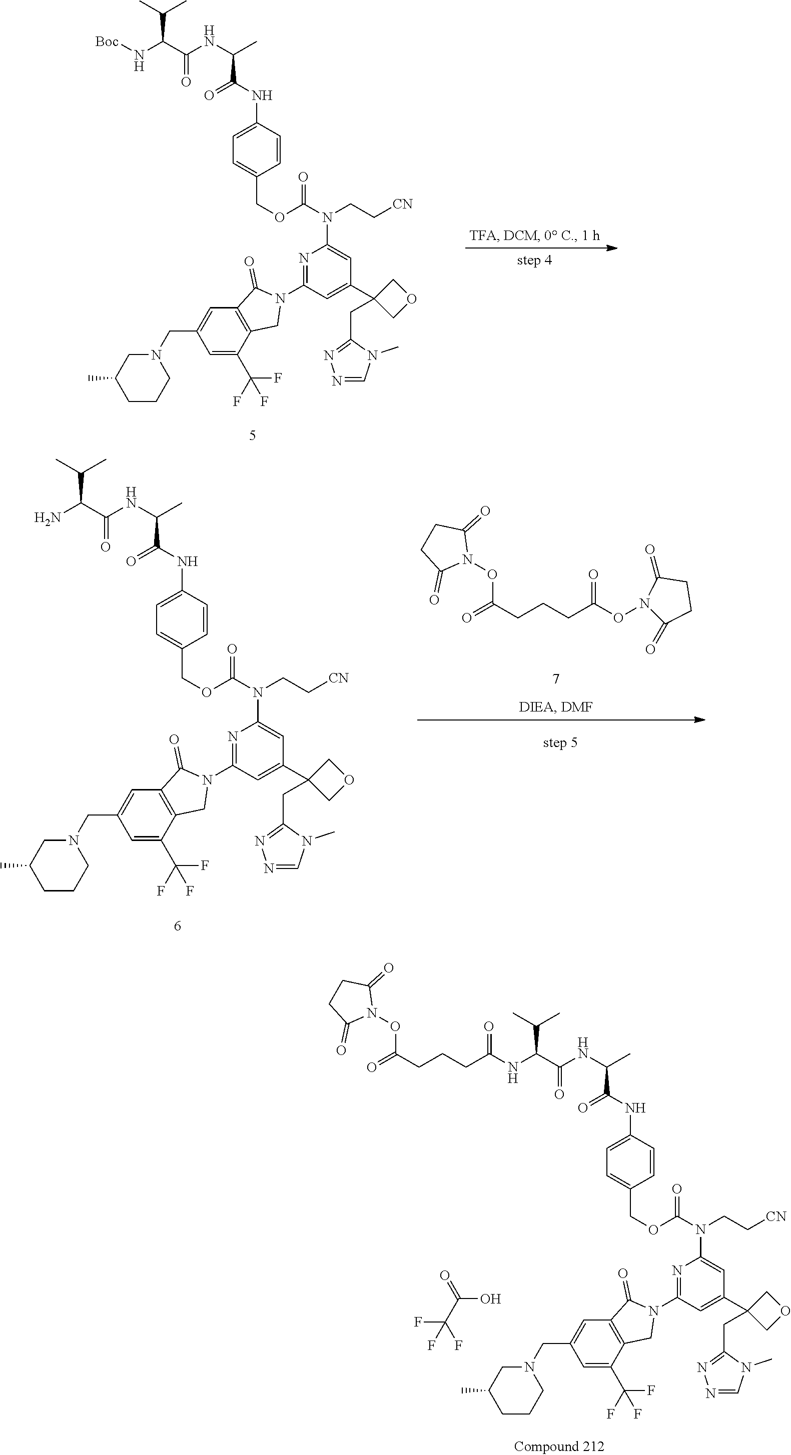

### Step 1: Synthesis of Compound 2

To a stirred solution of 2,6-dichloro-4-{3-[(4-methyl-1,2,4-triazol-3-yl)methyl]oxetan-3-yl}pyridine (INT3, 0.5 g, 1.67 mmol, 1.0 equiv) and β-aminopropionitrile (Compound 1, 2.35 g, 33.45 mmol, 20.02 equiv) in DMA (4.0 mL) was added K₂CO₃ (0.46 g, 3.34 mmol, 2.0 equiv) in portion at room temperature under nitrogen atmosphere. The resulting mixture was stirred for two days at 120° C. under nitrogen atmosphere. ˜70% desired product on LCMS. The reaction was cooled down to room temperature and quenched with water. The aqueous layer was extracted with CH₂Cl₂ (3×100 mL). The resulting mixture was concentrated under vacuum. The residue was purified by silica gel column chromatography, eluted with CH₂Cl₂/MeOH (5:1) to afford 3-[(6-chloro-4-{3-[(4-methyl-1,2,4-triazol-3-yl)methyl]oxetan-3-yl}pyridin-2-yl)amino]propanenitrile (Compound 2, 350 mg, 57%) as a light green solid. LCMS: (ES, m/s): 333,335 [M+H]⁺.

### Step 2: Synthesis of Compound 3

To a stirred solution of 6-{[(3S)-3-methylpiperidin-1-yl]methyl}-4-(trifluoromethyl)-2,3-dihydroisoindol-1-one (INT4, 330 mg, 1.05 mmol, 1.00 equiv) and 3-[(6-chloro-4-{3-[(4-methyl-1,2,4-triazol-3-yl)methyl]oxetan-3-yl}pyridin-2-yl)amino]propanenitrile (Compound 2, 350 mg, 1.05 mmol, 1 equiv) in dioxane (8 mL) was added Xantphos (120 mg, 0.20 mmol, 0.20 equiv), Pd(OAc)₂ (25 mg, 0.11 mmol, 0.11 equiv) and Cs₂CO₃ (860 mg, 2.64 mmol, 2.51 equiv) in portions at room temperature under nitrogen atmosphere. The resulting mixture was stirred for 1 h at 90° C. under nitrogen atmosphere. LCMS indicated the reaction was completed. The resulting mixture was filtered, the filter cake was washed with ACN and DCM. The filtrate was concentrated under reduced pressure. The residue was purified by silica gel column chromatography, eluted with CH₂Cl₂/MeOH (5:1) to afford 3-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]propanenitrile; formic acid (Compound 3, 330 mg, 43%) as a green solid. LCMS: (ES, m/s): 609 [M+H]⁺

### Step 3: Synthesis of Compound 5

To a stirred solution of 3-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]propanenitrile (Compound 3, 250 mg, 0.41 mmol, 1.0 equiv) in THE (25.00 mL) were added triphosgene (182 mg, 0.61 mmol, 1.5 equiv) in portions at room temperature under nitrogen atmosphere. The resulting mixture was stirred for additional 30 min at room temperature. The resulting mixture was concentrated under vacuum. The residue was re-dissolved in DCM (10 mL). To the above mixture was added tert-butyl N-[(1S)-1-{[(1S)-1-{[4-(hydroxymethyl)phenyl]carbamoyl}ethyl]carbamoyl}-2-methylpropyl]carbamate (Compound 4, 145 mg, 0.37 mmol, 0.9 equiv) and TEA (125 mg, 1.23 mmol, 3.0 equiv) at 0° C. The resulting mixture was stirred for additional 10 min at 0° C. Finally DMAP (100 mg, 0.82 mmol, 2.00 equiv) was added in portion. The resulting mixture was stirred for overnight at room temperature under nitrogen atmosphere. ˜10% desire product could be detected by LCMS. The resulting mixture was concentrated under reduced pressure. The residue was purified by reverse flash chromatography with the following conditions: column, C18 silica gel; mobile phase, MeCN in Water (0.1% TFA), 10% to 50% gradient in 30 min; detector, UV 254 nm. This resulted in {4-[(2S)-2-[(2S)-2-[(tert-butoxycarbonyl)amino]-3-methylbutanamido]propanamido]phenyl}methyl N-(2-cyanoethyl)-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (Compound 5, 120 mg, 28%) as a white solid. LCMS: (ES, m/s): 514 [M/2+H]⁺, 1028 [M+H]⁺

### Step 4: Synthesis of Compound 6

To a stirred solution of {4-[(2S)-2-[(2S)-2-[(tert-butoxycarbonyl)amino]-3-methylbutanamido]propanamido]phenyl}methyl N-(2-cyanoethyl)-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (Compound 5, 120 mg, 0.11 mmol, 1.0 equiv) in DCM (2.5 mL) were added TFA (0.5 mL) at 0° C. under nitrogen atmosphere. The resulting mixture was stirred for 2 h at room temperature under nitrogen atmosphere. LCMS indicated the reaction was completed. The resulting mixture was concentrated under vacuum. The crude product was used in the next step directly without further purification. LCMS: (ES, m/s): 465 [M/2+H]⁺, 928 [M+H]⁺

### Step 5: Synthesis of Compound 212

To a stirred mixture of {4-[(2S)-2-[(2S)-2-amino-3-methylbutanamido]propanamido]phenyl}methyl N-(2-cyanoethyl)-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (Compound 6, 25 mg, 0.027 mmol, 1 equiv) and bis(2,5-dioxopyrrolidin-1-yl) pentanedioate (Compound 7, 10 mg, 0.031 mmol, 1.14 equiv) in DMF (2 mL) was added DIEA (10 mg, 0.077 mmol, 2.87 equiv) dropwise at 0° C. under nitrogen atmosphere. The resulting mixture was stirred for 3 h at room temperature under nitrogen atmosphere. LCMS indicated the reaction was completed. The reaction mixture was purified by Prep-HPLC with the following conditions (Column: XSelect CSH Prep C18 OBD Column, 19*250 mm, 5 m; Mobile Phase A: water (0.05% TFA), Mobile Phase B: ACN; Flow rate: 25 mL/min; Gradient: 20% B to 40% B in 10 min, 40% B; Wave Length: 254 nm; RT1 (min): 9.58; The collected fraction was immediately lyophilized to afford 2,5-dioxopyrrolidin-1-yl 4-{[(1S)-1-{[(1 S)-1-{[4-({[(2-cyanoethyl)(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamoyl]oxy}methyl)phenyl]carbamoyl}ethyl]carbamoyl}-2-methylpropyl]carbamoyl}butanoate; trifluoroacetic acid (Compound 212, 6.3 mg, 18%) as a white solid. LCMS(ESI, m/z): 570 [M/2+H]⁺, 1139[M+H−TFA]⁺; ¹H NMR (300 MHz, DMSO-d₆) δ 10.10 (s, 1H), 9.85 (br s, 1H), 8.53 (s, 1H), 8.49-8.22 (m, 3H), 8.04-7.90 (m, 2H), 7.63 (d, J=8.4 Hz, 2H), 7.41-7.39 (m, 3H), 5.33 (s, 2H), 5.17 (s, 2H), 4.94-4.93 (m, 2H), 4.78-4.76 (m, 2H), 4.67-4.38 (m, 4H), 4.28-4.19 (m, 3H), 3.68 (s, 2H), 3.39-3.26 (m, 4H), 2.97-2.88 (m, 3H), 2.81 (s, 4H), 2.68-2.65 (m, 3H), 2.29-2.10 (m, 2H), 1.98-1.60 (m, 7H), 1.31-1.24 (m, 3H), 1.07-1.04 (m, 1H), 0.88-0.82 (m, 9H).

### Synthesis of Compound 213

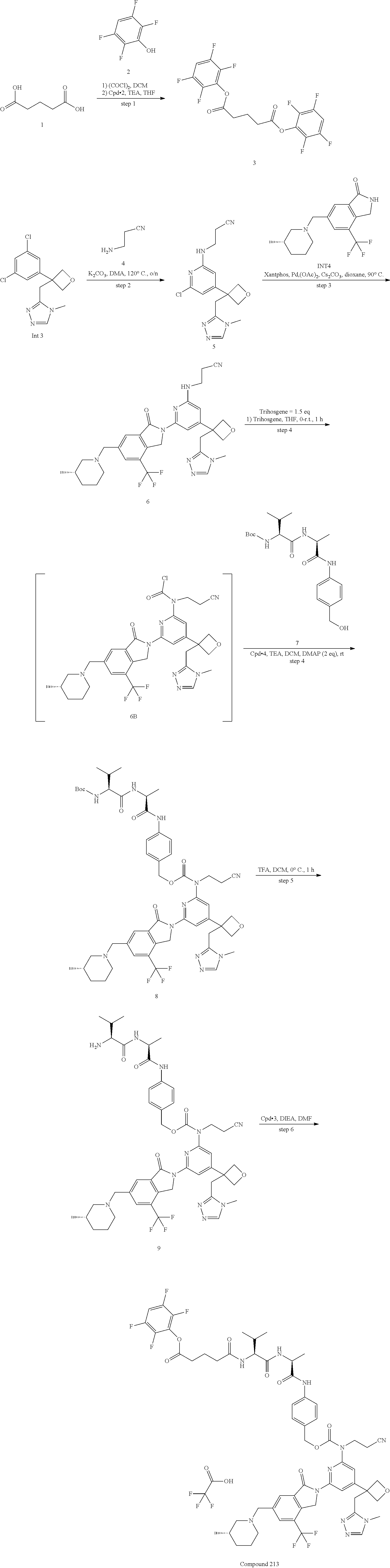

### Step 1: Synthesis of Compound 3

To a stirred mixture of glutaric acid (Compound 1, 200 mg, 1.51 mmol, 1 equiv) in DCM (10 mL) was added (COCl)₂ (384 mg, 3.02 mmol, 2 equiv) and DMF (3 drops) at 0° C. under nitrogen atmosphere. The resulting mixture was stirred for 3 h at room temperature. The resulting mixture was concentrated under reduced pressure. The residue was re-dissolved in THE (5 mL). To a stirred mixture of 2,3,5,6-tetrafluorophenol (Compound 2, 251 mg, 1.51 mmol, 1 equiv) and TEA (612 mg, 6.05 mmol, 4 equiv) in THE (10 mL) were added the above mixture dropwise at 0° C. under nitrogen atmosphere. The resulting mixture was stirred for overnight at room temperature. TLC showed a new spot. The resulting mixture was diluted with water (20 mL). The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EA (5:1) to afford bis(2,3,5,6-tetrafluorophenyl) pentanedioate (Compound 3, 100 mg, 13%) as a white solid. ¹H NMR (300 MHz, CDCl₃): δ 7.07-6.95 (m, 2H), 2.89-2.84 (m, 4H), 2.30-2.20 (m, 2H).

### Step 2: Synthesis of Compound 5

To a stirred solution of 2,6-dichloro-4-{3-[(4-methyl-1,2,4-triazol-3-yl)methyl]oxetan-3-yl}pyridine (INT3, 0.5 g, 1.67 mmol, 1.0 equiv) and β-aminopropionitrile (Compound 4, 2.35 g, 33.45 mmol, 20.02 equiv) in DMA (4.0 mL) was added K₂CO₃ (0.46 g, 3.34 mmol, 2.0 equiv) in portion at room temperature under nitrogen atmosphere. The resulting mixture was stirred for two days at 120° C. under nitrogen atmosphere. ˜70% desired product on LCMS. The reaction was cooled down to room temperature and quenched with water. The aqueous layer was extracted with CH₂Cl₂ (3×100 mL). The resulting mixture was concentrated under vacuum. The residue was purified by silica gel column chromatography, eluted with CH₂Cl₂/MeOH (5:1) to afford 3-[(6-chloro-4-{3-[(4-methyl-1,2,4-triazol-3-yl)methyl]oxetan-3-yl}pyridin-2-yl)amino]propanenitrile (Compound 5, 350 mg, 57%) as a light green solid. LCMS: (ES, m/s): 333,335 [M+H]⁺.

### Step 3: Synthesis of Compound 6

To a stirred solution of 6-{[(3S)-3-methylpiperidin-1-yl]methyl}-4-(trifluoromethyl)-2,3-dihydroisoindol-1-one (Compound 5, 330 mg, 1.05 mmol, 1.00 equiv) and 3-[(6-chloro-4-{3-[(4-methyl-1,2,4-triazol-3-yl)methyl]oxetan-3-yl}pyridin-2-yl)amino]propanenitrile (INT4, 350 mg, 1.05 mmol, 1 equiv) in dioxane (8 mL) was added Xantphos (120 mg, 0.20 mmol, 0.20 equiv), Pd(OAc)₂ (25 mg, 0.11 mmol, 0.11 equiv) and Cs₂CO₃ (860 mg, 2.64 mmol, 2.51 equiv) in portions at room temperature under nitrogen atmosphere. The resulting mixture was stirred for 1 h at 90° C. under nitrogen atmosphere. LCMS indicated the reaction was completed. The resulting mixture was filtered, the filter cake was washed with ACN and DCM. The filtrate was concentrated under reduced pressure. The residue was purified by silica gel column chromatography, eluted with CH₂Cl₂/MeOH (5:1) to afford 3-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]propanenitrile; formic acid (330 mg, 43%) as a green solid. LCMS: (ES, m/s): 609 [M+H]⁺

### Step 4: Synthesis of Compound 8

To a stirred solution of 3-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]propanenitrile (Compound 6, 250 mg, 0.41 mmol, 1.0 equiv) in THE (25.00 mL) was added triphosgene (182 mg, 0.61 mmol, 1.5 equiv) in portions at room temperature under nitrogen atmosphere. The resulting mixture was stirred for additional 30 min at room temperature. The resulting mixture was concentrated under vacuum. The residue was re-dissolved in DCM (10 mL). To the above mixture was added tert-butyl N-[(1S)-1-{[(1S)-1-{[4-(hydroxymethyl)phenyl]carbamoyl}ethyl]carbamoyl}-2-methylpropyl]carbamate (Compound 7, 145 mg, 0.37 mmol, 0.9 equiv) and TEA (125 mg, 1.23 mmol, 3.0 equiv) at 0° C. The resulting mixture was stirred for additional 10 min at 0° C. Finally DMAP (100 mg, 0.82 mmol, 2.00 equiv) was added in portion. The resulting mixture was stirred for overnight at room temperature under nitrogen atmosphere. ˜10% desire product could be detected by LCMS. The resulting mixture was concentrated under reduced pressure. The residue was purified by reverse flash chromatography with the following conditions: column, C18 silica gel; mobile phase, MeCN in water (0.1% TFA), 10% to 50% gradient in 30 min; detector, UV 254 nm. This resulted in {4-[(2S)-2-[(2S)-2-[(tert-butoxycarbonyl)amino]-3-methylbutanamido]propanamido]phenyl}methyl N-(2-cyanoethyl)-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (Compound 8, 120 mg, 28%) as a white solid. LCMS: (ES, m/s): 514 [M12+H]⁺, 1028 [M+H]⁺

### Step 5: Synthesis of Compound 9

To a stirred solution of {4-[(2S)-2-[(2S)-2-[(tert-butoxycarbonyl)amino]-3-methylbutanamido]propanamido]phenyl}methyl N-(2-cyanoethyl)-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (Compound 8, 120 mg, 0.11 mmol, 1.0 equiv) in DCM (2.5 mL) were added TFA (0.5 mL) at 0° C. under nitrogen atmosphere. The resulting mixture was stirred for 2 h at room temperature under nitrogen atmosphere. LCMS indicated the reaction was completed. The resulting mixture was concentrated under vacuum. The crude product was used in the next step directly without further purification. LCMS: (ES, m/s): 465 [M/2+H]⁺, 928 [M+H]⁺

### Step 6: Synthesis of Compound 213

To a stirred mixture of 4-{[(1S)-1-{[(1S)-1-{[4-({[(2-cyanoethyl)(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamoyl]oxy}methyl)phenyl]carbamoyl}ethyl]carbamoyl}-2-methylpropyl]carbamoyl}butanoic acid (25 mg, 0.024 mmol, 1 equiv) and HATU (10 mg, 0.029 mmol, 1.2 equiv) in DMF (1 mL) was added HOBT (3 mg, 0.024 mmol, 1 equiv), bis(2,3,5,6-tetrafluorophenyl) pentanedioate (Compound 3, 4 mg, 0.024 mmol, 1 equiv) and DIEA (6 mg, 0.048 mmol, 2 equiv) at 0° C. under nitrogen atmosphere. The resulting mixture was stirred for 3 h at 0° C. under nitrogen atmosphere. LCMS indicated the reaction was completed. The reaction mixture was purified by Prep-HPLC with the following conditions (Column: XSelect CSH Prep C18 OBD Column, 19*250 mm, 5 m; Mobile Phase A: Water (0.05% TFA), Mobile Phase B: ACN; Flow rate: 25 mL/min; Gradient: 22% B to 45% B in 10 min, 45% B to 45% B in 12 min, 45% B; Wave Length: 254 nm; RT1 (min): 11.59; The collected fraction was immediately lyophilized to afford 2,3,5,6-tetrafluorophenyl 4-{[(1S)-1-{[(1S)-1-{[4-({[(2-cyanoethyl)(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamoyl]oxy}methyl)phenyl]carbamoyl}ethyl]carbamoyl}-2-methylpropyl]carbamoyl}butanoate; trifluoroacetic acid (Compound 213, 8.3 mg, 26%) as a white solid. LCMS(ESI, m/z): 596 [M/2+H]⁺, 1190[M+H−TFA]⁺; ¹H NMR (300 MHz, DMSO-d₆) δ 10.10 (s, 1H), 9.85 (br s, 1H), 8.36-7.92 (m, 6H), 7.64 (d, J=8.4 Hz, 2H), 7.41-7.39 (m, 2H), 5.33 (s, 2H), 5.16 (s, 2H), 4.95-4.93 (m, 2H), 4.78-4.76 (m, 2H), 4.53 (s, 2H), 4.41-4.17 (m, 4H), 3.54 (s, 2H), 3.39-3.36 (m, 1H), 3.25 (s, 3H), 2.99-2.73 (m, 5H), 2.55-2.50 (m, 2H), 2.31-2.17 (m, 2H), 1.99-1.62 (m, 7H), 1.32-1.04 (m, 4H), 0.89-0.83 (m, 9H).

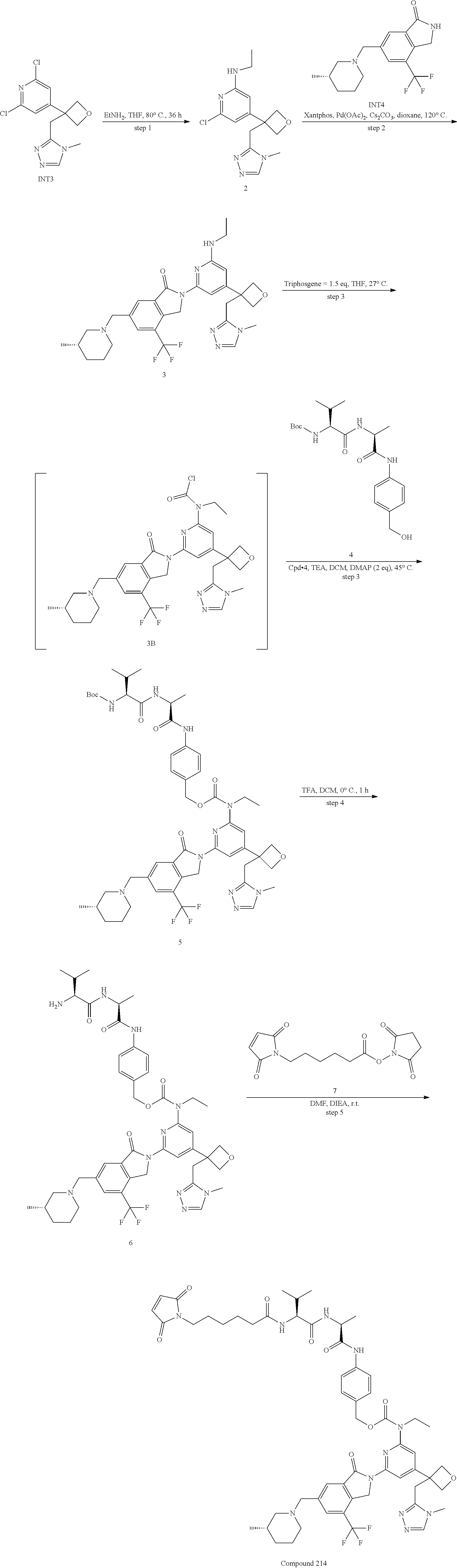

### Step 1. Synthesis of Compound 2:

A solution of 2,6-dichloro-4-{3-[(4-methyl-1,2,4-triazol-3-yl)methyl]oxetan-3-yl}pyridine (INT3, 1.55 g, 5.18 mmol, 1 equiv) and in ethylamine solution (2.0 M in THF, 15 mL) was stirred for overnight at 90° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (10:1) to afford 6-chloro-N-ethyl-4-{3-[(4-methyl-1,2,4-triazol-3-yl)methyl]oxetan-3-yl}pyridin-2-amine (Compound 2, 798 mg, 50%) as a yellow solid. LCMS (ESI, ms): 308,310[M+H]⁺

### Step 2: Synthesis of Compound 3

To a stirred solution of 6-chloro-N-ethyl-4-{3-[(4-methyl-1,2,4-triazol-3-yl)methyl]oxetan-3-yl}pyridin-2-amine (Compound 2, 200 mg, 0.65 mmol, 1.0 equiv) and 6-{[(3S)-3-methylpiperidin-1-yl]methyl}-4-(trifluoromethyl)-2,3-dihydroisoindol-1-one (INT4, 203 mg, 0.65 mmol, 1.0 equiv) in dioxane (8.0 mL) was added Xantphos (150 mg, 0.26 mmol, 0.4 equiv), Pd(OAc)₂ (58 mg, 0.26 mmol, 0.4 equiv) and Cs₂CO₃ (635 mg, 1.95 mmol, 3.0 equiv) in portions at room temperature under nitrogen atmosphere. The resulting mixture was stirred for 1 h at 120° C. under nitrogen atmosphere. LCMS indicated the reaction was completed. The mixture was allowed to cool down to room temperature and filtered, the filter cake was washed with ACN. The filtrate was concentrated under reduced pressure. The residue was purified by reverse flash chromatography with the following conditions: column, C18 silica gel; mobile phase, ACN in water (0.1% FA), 10% to 50% gradient in 30 min; detector, UV 254 nm and 220 nm. The collected fraction was concentrated under vacuum to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(trifluoromethyl)-3H-isoindol-1-one; formic acid (Compound 3, 260 mg, 61%) as a green solid. LCMS: (ES, m/s): 584 [M+H]⁺. ¹H-NMR (300 MHz, DMSO-d₆): 8.25 (s, 1H), 8.15 (s, 1H), 7.95 (s, 1H), 7.91 (s, 1H), 7.41 (s, 1H), 6.62 (t, J=5.4 Hz, 1H), 5.89 (s, 1H), 5.14 (s, 2H), 4.90 (d, J=6 Hz, 2H), 4.78 (d, J=6 Hz, 2H), 3.65 (s, 3H), 3.49 (s, 3H), 3.42-3.11 (m, 5H), 2.75-2.66 (m, 2H), 1.95-1.93 (m, 1H), 1.70-1.53 (m, 4H), 1.53-1.40 (m, 1H), 1.13 (t, J=6 Hz, 3H), 0.80 (d, J=3 Hz, 3H).

### Step 3: Synthesis of Compound 5

To a stirred solution of 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(trifluoromethyl)-3H-isoindol-1-one (Compound 3, 200 mg, 0.34 mmol, 1.00 equiv) in THE (5 mL) was added triphosgene (200 mg, 0.67 mmol, 1.97 equiv) at room temperature. The resulting mixture was stirred for additional 30 min at room temperature. The resulting mixture was concentrated under vacuum. The residue was re-dissolved in DCM (5 mL). To the above mixture was added tert-butyl N-[(1S)-1-{[(1S)-1-{[4-(hydroxymethyl)phenyl]carbamoyl}-ethyl]carbamoyl}-2-methylpropyl]carbamate (Compound 4, 200 mg, 0.50 mmol, 1.48 equiv), TEA (200 mg, 1.97 mmol, 5.77 equiv) and DMAP (83 mg, 0.68 mmol, 2.0 equiv) in portions at room temperature under nitrogen atmosphere. The resulting mixture was stirred for overnight at 50° C. under nitrogen atmosphere. ˜20% desire product could be detected by LCMS. The reaction mixture was purified by reverse flash chromatography with the following conditions: column, C18 silica gel; mobile phase, ACN in water (0.1% FA), 0% to 70% gradient in 30 min; detector, UV 254 nm. The collected fraction was concentrated under vacuum to afford {4-[(2S)-2-[(2S)-2-[(tert-butoxycarbonyl)amino]-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (Compound 5, 100 mg, 26%) as a semi-solid. LCMS: (ES, m/s): 502 [M/2+H]⁺, 1003 [M+H]⁺.

### Step 4: Synthesis of Compound 6

To a stirred solution of {4-[(2S)-2-[(2S)-2-[(tert-butoxycarbonyl)amino]-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (Compound 5, 100 mg, 0.10 mmol, 1.00 equiv) in DCM (2.00 mL) were added TFA (0.4 mL) in portions at room temperature under nitrogen atmosphere. The resulting mixture was stirred for 2 h at room temperature under nitrogen atmosphere. LCMS indicated the reaction was completed. The resulting mixture was concentrated under vacuum. The residue was purified by reverse flash chromatography with the following conditions: column, C18 silica gel; mobile phase, ACN in water (0.05% TFA), 10% to 50% gradient in 30 min; detector, UV 254 nm. The collected fraction was lyophilized to afford {4-[(2S)-2-[(2S)-2-amino-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (Compound 6, 100 mg, 91%) as a white solid. LCMS: (ES, m/s): 903 [M+H], 925 [M+Na]⁺.

### Step 5: Synthesis of Compound 214

To a stirred solution of {4-[(2S)-2-[(2S)-2-amino-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (100 mg, 0.091 mmol, 1.00 equiv, 82%) in DMF (2 mL) were added 2,5-dioxopyrrolidin-1-yl 6-(2,5-dioxopyrrol-1-yl)hexanoate (Compound 7, 32 mg, 0.10 mmol, 1.17 equiv) and DIEA (36 mg, 0.28 mmol, 3.06 equiv) in portions at room temperature under nitrogen atmosphere. The resulting mixture was stirred for 2 h at room temperature under nitrogen atmosphere. LCMS indicated the reaction was completed. The reaction mixture was purified by Prep-HPLC with the following conditions (Column: Xselect Peptide CSH C18 19*150 mm 5 m, 1; Mobile Phase A: Water (0.1% FA), Mobile Phase B: MeOH--HPLC; Flow rate: 25 mL/min; Gradient: 50% B to 60% B in 14 min, 60% B to 60% B in 16 min, 60% B; Wave Length: 254 nm; RT1 (min): 14.3; The collected fraction was lyophilized to afford {4-[(2S)-2-[(2S)-2-[6-(2,5-dioxopyrrol-1-yl)hexanamido]-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (Compound 214, 5.1 mg, 5%) as a white solid. LCMS: (ES, m/s): 549 [M/2+H]⁺, 1096 [M+H]⁺. H-NMR (300 MHz, DMSO-d₆): 10.02 (s, 1H), 8.40-8.22 (m, 2H), 7.98-7.94 (m, 3H), 7.84 (d, J=8.4 Hz, 1H), 7.62 (d, J=8.4 Hz, 2H), 7.38 (d, J=8.4 Hz, 2H), 7.23 (s, 1H), 6.99 (s, 2H), 5.18-5.13 (m, 4H), 4.94 (d, J=6 Hz, 2H), 4.78 (d, J=6.3 Hz, 2H), 4.35 (t, J=6.0 Hz, 1H), 4.19 (t, J=6.0 Hz, 1H), 3.92-3.87 (m, 2H), 3.64 (s, 2H), 3.50 (s, 2H), 3.30-3.28 (m, 2H), 3.19 (s, 3H), 2.75-2.60 (m, 2H), 2.27-2.10 (m, 2H), 2.08-1.89 (m, 2H), 1.70-1.44 (m, 9H), 1.35-1.15 (m, 9H), 0.92-0.80 (m, 9H).

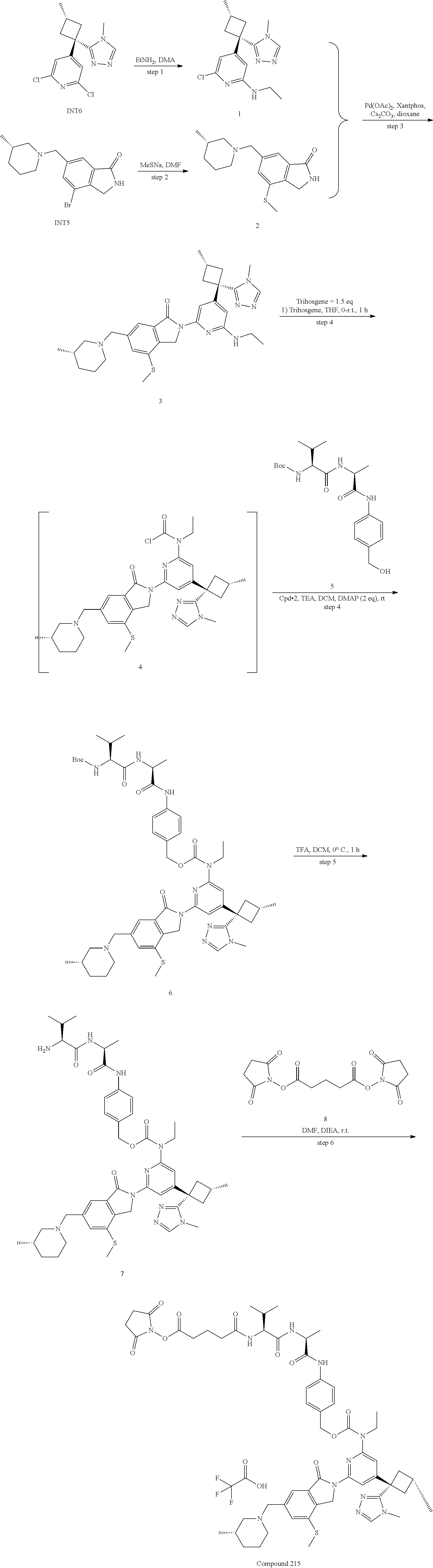

### Step 1: Synthesis of Compound 1

To a stirred solution of 2,6-dichloro-4-[3-methyl-1-(4-methyl-1,2,4-triazol-3-yl)cyclobutyl]pyridine (3 g, 10.09 mmol, 1 equiv) in Ethylamine solution 2.0 M in THF (30 mL) was stirred for 2 days at 90° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (12:1) to afford 6-chloro-N-ethyl-4-[(1r,3s)-3-methyl-1-(4-methyl-1,2,4-triazol-3-yl)cyclobutyl]pyridin-2-amine (2.1 g, 66%) as an off-white solid. LCMS (ESI, m/z): 306,308[M+H]⁺

### Step 2: Synthesis of Compound 2

To a stirred solution of 4-bromo-6-{[(3S)-3-methylpiperidin-1-yl]methyl}-2,3-dihydroisoindol-1-one (500 mg, 1.54 mmol, 1 equiv) in DMF (5 mL) was added Sodiummethanethiolate (162 mg, 2.32 mmol, 1.5 equiv) in portions at room temperature under nitrogen atmosphere. The resulting mixture was stirred for 1 h at 90° C. under nitrogen atmosphere. LCMS indicated the reaction was completed. The mixture was allowed to cool down to room temperature. The residue was purified by reverse flash chromatography with the following conditions: column, C18 silica gel; mobile phase, MeCN in Water (0.05% TFA), 5% to 60% gradient in 30 min; detector, UV 254 nm. The collected fraction was concentrated to afford 6-{[(3S)-3-methylpiperidin-1-yl]methyl}-4-(methylsulfanyl)-2,3-dihydroisoindol-1-one (410 mg, 91%) as a white solid. LCMS (ESI, m/z): 291[M+H]⁺

### Step 3: Synthesis of Compound 3

To a stirred mixture of 6-chloro-N-ethyl-4-[(1r,3s)-3-methyl-1-(4-methyl-1,2,4-triazol-3-yl)cyclobutyl]pyridin-2-amine (1 g, 3.27 mmol, 1 equiv) and 6-{[(3S)-3-methylpiperidin-1-yl]methyl}-4-(methylsulfanyl)-2,3-dihydroisoindol-1-one (0.95 g, 3.27 mmol, 1 equiv) in dioxane (10 mL) were added Pd(OAc)₂ (0.07 g, 0.32 mmol, 0.1 equiv) and Xantphos (0.38 g, 0.65 mmol, 0.2 equiv) and Cs₂CO₃ (2.13 g, 6.54 mmol, 2 equiv) at room temperature under nitrogen atmosphere. The resulting mixture was stirred for 1 h at 120° C. under nitrogen atmosphere. LCMS indicated the reaction was completed. The mixture was allowed to cool down to room temperature. The resulting mixture was concentrated under reduced pressure. The residue was purified by silica gel column chromatography, eluted with CH₂Cl₂/MeOH (10:1) to afford 2-[6-(ethylamino)-4-[(1r,3s)-3-methyl-1-(4-methyl-1,2,4-triazol-3-yl)cyclobutyl]pyridin-2-yl]-6-{[(3S)-3-methylpiperidin-1-yl]methyl}-4-(methylsulfanyl)-3H-isoindol-1-one (360 mg, 20%) as a white solid. LCMS (ES, m/z): 560 [M+H]⁺

### Step 4: Synthesis of Compound 6

To a stirred solution of 2-[6-(ethylamino)-4-[(1r,3s)-3-methyl-1-(4-methyl-1,2,4-triazol-3-yl)cyclobutyl]pyridin-2-yl]-6-{[(3S)-3-methylpiperidin-1-yl]methyl}-4-(methylsulfanyl)-3H-isoindol-1-one (360 mg, 0.64 mmol, 1 equiv) and ditrichloromethyl carbonate (286 mg, 0.96 mmol, 1.5 equiv) in THE (32 mL) was stirred for 1 h at room temperature under nitrogen atmosphere. LCMS indicated the reaction was completed. The resulting mixture was concentrated under vacuum to afford crude product 4. To a stirred solution of tert-butyl N-[(1S)-1-{[(1S)-1-{[4-(hydroxymethyl)phenyl]carbamoyl}ethyl]carbamoyl}-2-methylpropyl]carbamate (253 mg, 0.64 mmol, 1 equiv) and TEA (195 mg, 1.92 mmol, 3 equiv) in DCM (10 mL) was added the above residue(crude product 4) in DCM (3 mL) and DMAP (157 mg, 1.28 mmol, 2 equiv) at room temperature under nitrogen atmosphere. The resulting mixture was stirred for overnight at 40° C. under nitrogen atmosphere. LCMS indicated the reaction was completed. The mixture was allowed to cool down to room temperature. The resulting mixture was concentrated under vacuum. The residue was purified by reversed-phase flash chromatography with the following conditions: column, C18 silica gel; mobile phase, MeCN in Water (0.1% TFA), 5% to 80% gradient in 30 min; detector, UV 254 nm. The collected fraction was concentrated under vacuum and lyophilized to afford {4-[(2S)-2-[(2S)-2-[(tert-butoxycarbonyl)amino]-3-methylbutanamido]propanamido]phenyl}methyl N-ethyl-N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate (270 mg, 42%) as an off-white solid. LCMS (ESI, m/z):979[M+H]⁺

### Step 5: Synthesis of Compound 7

To a stirred solution of {4-[(2S)-2-[(2S)-2-[(tert-butoxycarbonyl)amino]-3-methylbutanamido]propanamido]phenyl}methyl N-ethyl-N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate (270 mg, 0.27 mmol, 1 equiv) in DCM (5 mL) was added TFA (1 mL) dropwise at 0° C. The resulting mixture was stirred for 2 h at 0° C. under nitrogen atmosphere. LCMS indicated the reaction was completed. The resulting mixture was concentrated under vacuum. The product was used to next step without further purification. LCMS (ESI, m/z):879[M+H]⁺

### Step 6: Synthesis of Compound 215

To a stirred solution of {4-[(2S)-2-[(2S)-2-amino-3-methylbutanamido]propanamido]phenyl}methyl N-ethyl-N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate (100 mg, 0.11 mmol, 1 equiv) and bis(2,5-dioxopyrrolidin-1-yl) pentanedioate (44 mg, 0.13 mmol, 1.2 equiv) in DMF (2 mL) was added DIEA (79 uL, 0.45 mmol, 4 equiv) dropwise at 0° C. The resulting mixture was stirred for 2 h at 0° C. under nitrogen atmosphere. LCMS indicated the reaction was completed. The reaction mixture was purified by Prep-HPLC with the following conditions (Column: XSelect CSH Prep C18 OBD Column, 19*150 mm, 5 m; Mobile Phase A: Water (0.1% TFA), Mobile Phase B: ACN; Flow rate: 25 mL/min; Gradient: 24% B to 49% B in 10 min, 49% B; Wave Length: 254 nm; RT1 (min): 6.93; The collected fraction was immediately lyophilized to afford 2,5-dioxopyrrolidin-1-yl 4-{[(1S)-1-{[(1S)-1-({4-[({ethyl[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamoyl}oxy)methyl]phenyl}carbamoyl)ethyl]carbamoyl}-2-methylpropyl]carbamoyl}butanoate; trifluoroacetic acid (46.1 mg, 32%) as a white solid. LCMS (ESI, m/z):1090[M+H−TFA]^{+ 1}H NMR (400 MHz, DMSO-d₆) δ10.01 (m, 2H), 9.62 (br s, 1H), 8.49 (s, 1H), 8.22 (d, J=6.8 Hz, 1H), 8.11 (s, 1H), 7.90 (d, J=8.4 Hz, 1H), 7.71-7.52 (m, 5H), 7.38 (d, J=8.4 Hz, 2H), 5.14 (s, 2H), 4.94 (s, 2H), 4.42-4.37 (m, 3H), 4.21-4.19 (m, 1H), 3.98-3.97 (m, 2H), 3.40-2.35 (m, 1H), 3.32-3.25 (m, 1H), 3.20 (s, 3H), 2.81-2.77 (m, 5H), 2.70-2.62 (m, 7H), 2.25-2.50 (m, 3H), 2.49-2.48 (m, 1H), 2.32-2.28 (m, 2H), 2.00-1.90 (m, 1H), 1.86-65 (m, 6H), 1.31-1.26 (m, 6H), 1.07-1.05 (m, 4H), 0.89-0.83 (m, 9H).

### Synthesis of Compound 216

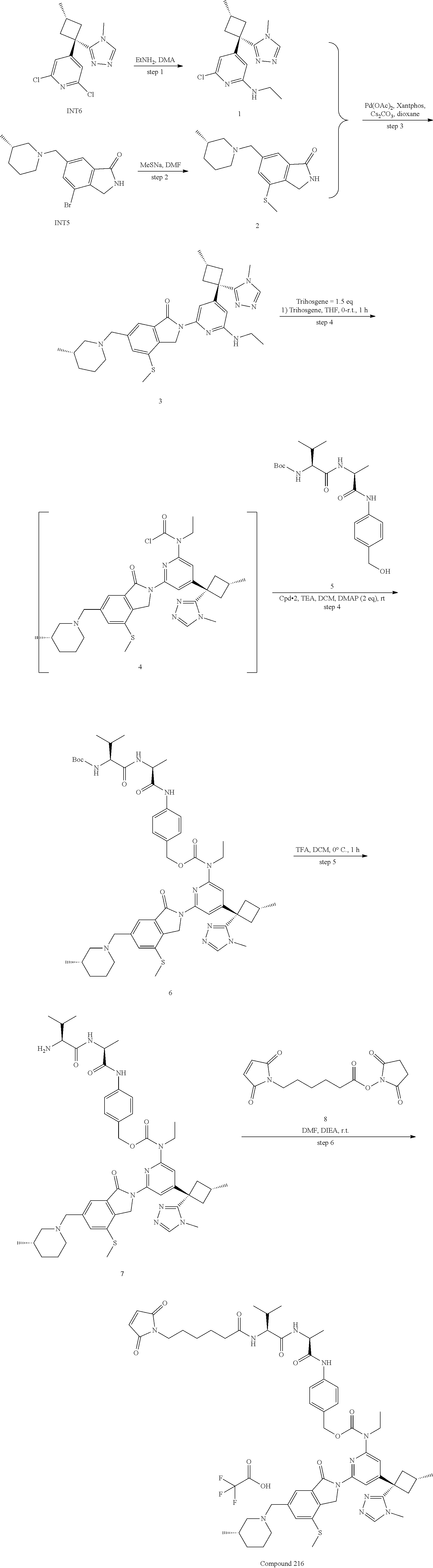

### Step 1. Synthesis of Compound 1

To a stirred solution of 2,6-dichloro-4-[3-methyl-1-(4-methyl-1,2,4-triazol-3-yl)cyclobutyl]pyridine (3 g, 10.09 mmol, 1 equiv) in Ethylamine solution 2.0 M in THF (30 mL) was stirred for 2 days at 90° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (12:1) to afford 6-chloro-N-ethyl-4-[(1r,3s)-3-methyl-1-(4-methyl-1,2,4-triazol-3-yl)cyclobutyl]pyridin-2-amine (2.1 g, 66%) as an off-white solid. LCMS (ESI, m/z): 306,308[M+H]⁺

### Step 2: Synthesis of Compound 2

To a stirred solution of 4-bromo-6-{[(3S)-3-methylpiperidin-1-yl]methyl}-2,3-dihydroisoindol-1-one (500 mg, 1.54 mmol, 1 equiv) in DMF (5 mL) was added Sodiummethanethiolate (162 mg, 2.32 mmol, 1.5 equiv) in portions at room temperature under nitrogen atmosphere. The resulting mixture was stirred for 1 h at 90° C. under nitrogen atmosphere. LCMS indicated the reaction was completed. The mixture was allowed to cool down to room temperature. The residue was purified by reverse flash chromatography with the following conditions: column, C18 silica gel; mobile phase, MeCN in Water (0.05% TFA), 5% to 60% gradient in 30 min; detector, UV 254 nm. The collected fraction was concentrated to afford 6-{[(3S)-3-methylpiperidin-1-yl]methyl}-4-(methylsulfanyl)-2,3-dihydroisoindol-1-one (410 mg, 91%) as a white solid. LCMS (ESI, m/z): 291[M+H]⁺

### Step 3: Synthesis of Compound 3

To a stirred mixture of 6-chloro-N-ethyl-4-[(1r,3s)-3-methyl-1-(4-methyl-1,2,4-triazol-3-yl)cyclobutyl]pyridin-2-amine (1 g, 3.27 mmol, 1 equiv) and 6-{[(3S)-3-methylpiperidin-1-yl]methyl}-4-(methylsulfanyl)-2,3-dihydroisoindol-1-one (0.95 g, 3.27 mmol, 1 equiv) in dioxane (10 mL) were added Pd(OAc)₂ (0.07 g, 0.32 mmol, 0.1 equiv) and Xantphos (0.38 g, 0.65 mmol, 0.2 equiv) and Cs₂CO₃ (2.13 g, 6.54 mmol, 2 equiv) at room temperature under nitrogen atmosphere. The resulting mixture was stirred for 1 h at 120° C. under nitrogen atmosphere. LCMS indicated the reaction was completed. The mixture was allowed to cool down to room temperature. The resulting mixture was concentrated under reduced pressure. The residue was purified by silica gel column chromatography, eluted with CH₂Cl₂/MeOH (10:1) to afford 2-[6-(ethylamino)-4-[(1r,3s)-3-methyl-1-(4-methyl-1,2,4-triazol-3-yl)cyclobutyl]pyridin-2-yl]-6-{[(3S)-3-methylpiperidin-1-yl]methyl}-4-(methylsulfanyl)-3H-isoindol-1-one (360 mg, 20%) as a white solid. LCMS (ES, m/z): 560 [M+H]⁺

### Step 4: Synthesis of Compound 6

To a stirred solution of 2-[6-(ethylamino)-4-[(1r,3s)-3-methyl-1-(4-methyl-1,2,4-triazol-3-yl)cyclobutyl]pyridin-2-yl]-6-{[(3S)-3-methylpiperidin-1-yl]methyl}-4-(methylsulfanyl)-3H-isoindol-1-one (360 mg, 0.64 mmol, 1 equiv) and ditrichloromethyl carbonate (286 mg, 0.96 mmol, 1.5 equiv) in THE (32 mL) was stirred for 1 h at room temperature under nitrogen atmosphere. LCMS indicated the reaction was completed. The resulting mixture was concentrated under vacuum to afford crude product 4. To a stirred solution of tert-butyl N-[(1S)-1-{[(1S)-1-{[4-(hydroxymethyl)phenyl]carbamoyl}ethyl]carbamoyl}-2-methylpropyl]carbamate (253 mg, 0.64 mmol, 1 equiv) and TEA (195 mg, 1.92 mmol, 3 equiv) in DCM (10 mL) was added the above residue(crude product 4) in DCM (3 mL) and DMAP (157 mg, 1.28 mmol, 2 equiv) at room temperature under nitrogen atmosphere. The resulting mixture was stirred for overnight at 40° C. under nitrogen atmosphere. LCMS indicated the reaction was completed. The mixture was allowed to cool down to room temperature. The resulting mixture was concentrated under vacuum. The residue was purified by reversed-phase flash chromatography with the following conditions: column, C18 silica gel; mobile phase, MeCN in Water (0.1% TFA), 5% to 80% gradient in 30 min; detector, UV 254 nm. The collected fraction was concentrated under vacuum and lyophilized to afford {4-[(2S)-2-[(2S)-2-[(tert-butoxycarbonyl)amino]-3-methylbutanamido]propanamido]phenyl}methyl N-ethyl-N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate (270 mg, 42%) as an off-white solid. LCMS (ESI, m/z):979[M+H]⁺

### Step 5: Synthesis of Compound 7

To a stirred solution of {4-[(2S)-2-[(2S)-2-[(tert-butoxycarbonyl)amino]-3-methylbutanamido]propanamido]phenyl}methyl N-ethyl-N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate (270 mg, 0.27 mmol, 1 equiv) in DCM (5 mL) was added TFA (1 mL) dropwise at 0° C. The resulting mixture was stirred for 2 h at 0° C. under nitrogen atmosphere. LCMS indicated the reaction was completed. The resulting mixture was concentrated under vacuum. The product was used to next step without further purification. LCMS (ESI, m/z):879[M+H]⁺

### Step 6: Synthesis of Compound 216

To a stirred solution of {4-[(2S)-2-[(2S)-2-amino-3-methylbutanamido]propanamido]phenyl}methyl N-ethyl-N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate (100 mg, 0.11 mmol, 1 equiv) and 2,5-dioxopyrrolidin-1-yl 6-(2,5-dioxopyrrol-1-yl)hexanoate (42 mg, 0.13 mmol, 1.2 equiv) in DMF (2 mL) was added DIEA (79 uL, 0.45 mmol, 4 equiv) dropwise at 0° C. The resulting mixture was stirred for 2 h at 0° C. under nitrogen atmosphere. LCMS indicated the reaction was completed. The reaction mixture was purified by Prep-HPLC with the following conditions (Column: XSelect CSH Prep C18 OBD Column, 19*150 mm, 5 m; Mobile Phase A: Water (0.1% TFA), Mobile Phase B: ACN; Flow rate: 25 mL/min; Gradient: 27% B to 57% B in 7 min, 57% B; Wave Length: 254 nm; RT1 (min): 5.13; The reaction mixture was lyophilized to afford {4-[(2S)-2-[(2S)-2-[6-(2,5-dioxopyrrol-1-yl)hexanamido]-3-methylbutanamido]propanamido]phenyl}methyl N-ethyl-N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate; trifluoroacetic acid (61 mg, 49%) as a white solid. LCMS (ESI, m/z):1072[M+H−TFA]^{+ 1}H NMR (400 MHz, CD₃OD) δ 8.76 (s, 1H), 8.28 (s, 1H), 7.74 (s, 1H), 7.70 (s, 1H), 7.64 (d, J=8.4, 2H), 7.53 (s, 1H), 7.39 (d, J=8.4 Hz, 2H), 6.78 (s, 2H), 5.20 (s, 2H), 5.01 (s, 2H), 4.46-4.45 (m, 3H), 4.15-4.08 (m, 3H), 3.49-3.45 (m, 4H), 3.32 (s, 3H), 2.92-2.81 (m, 3H), 2.68-2.65 (m, 7H), 2.29-2.27 (m, 2H), 2.12-2.05 (m, 1H), 2.01-1.70 (m, 4H), 1.68-1.52 (m, 4H), 1.47 (d, J=6.8 Hz, 3H), 1.37-1.34 (m, 6H), 1.16 (d, J=5.2 Hz, 3H), 1.01-0.97 (m, 9H).

### Synthesis of Compound 217

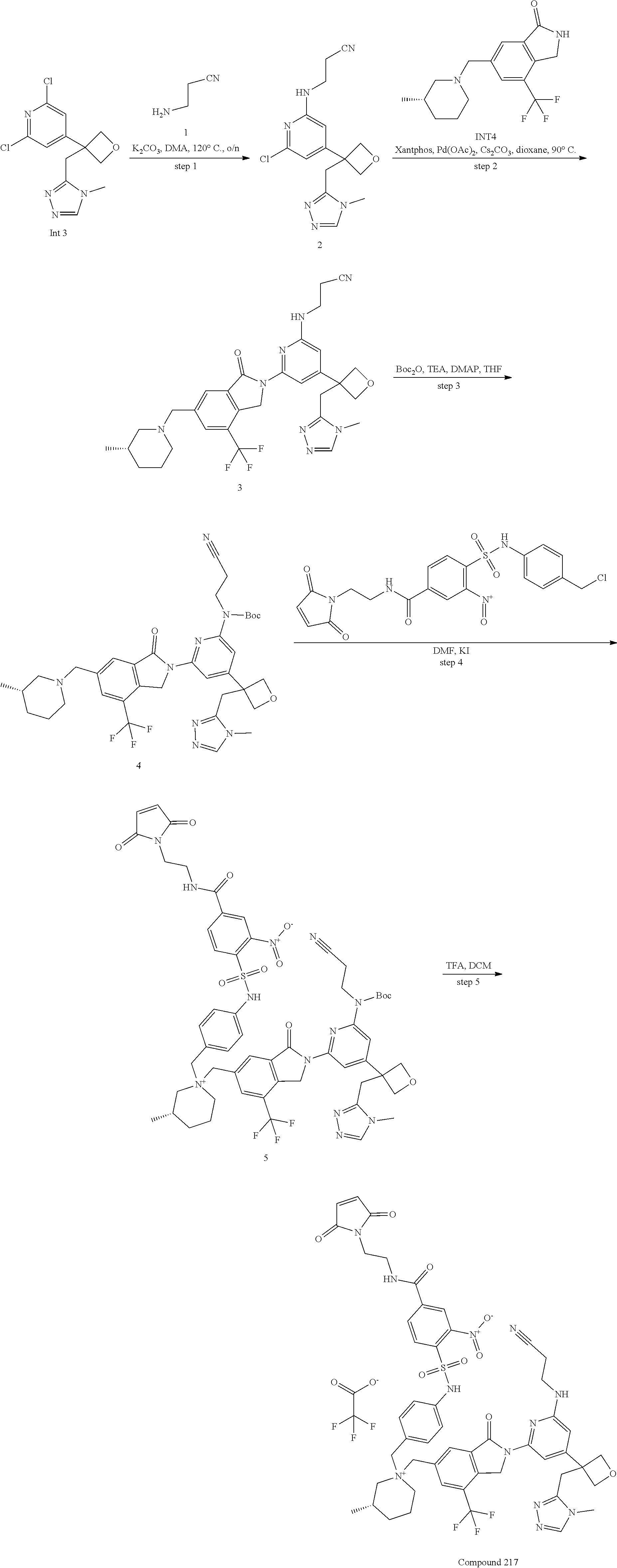

### Step 1: Synthesis of Compound 2

To a stirred solution of 2,6-dichloro-4-{3-[(4-methyl-1,2,4-triazol-3-yl)methyl]oxetan-3-yl}pyridine (0.5 g, 1.67 mmol, 1.0 equiv) and β-aminopropionitrile (2.35 g, 33.45 mmol, 20.02 equiv) in DMA (4.0 mL) was added K₂CO₃ (0.46 g, 3.34 mmol, 2.0 equiv) in portion at room temperature under nitrogen atmosphere. The resulting mixture was stirred for two days at 120° C. under nitrogen atmosphere. ˜₇₀% desired product on LCMS. The reaction was cooled down to room temperature and quenched with water. The aqueous layer was extracted with CH₂Cl₂ (3×100 mL). The resulting mixture was concentrated under vacuum. The residue was purified by silica gel column chromatography, eluted with CH₂Cl₂/MeOH (5:1) to afford 3-[(6-chloro-4-{3-[(4-methyl-1,2,4-triazol-3-yl)methyl]oxetan-3-yl}pyridin-2-yl)amino]propanenitrile (350 mg, 57%) as a light green solid. LCMS: (ES, m/s): 333,335 [M+H]⁺.

### Step 2: Synthesis of Compound 3

To a stirred solution of 6-{[(3S)-3-methylpiperidin-1-yl]methyl}-4-(trifluoromethyl)-2,3-dihydroisoindol-1-one (330 mg, 1.05 mmol, 1.00 equiv) and 3-[(6-chloro-4-{3-[(4-methyl-1,2,4-triazol-3-yl)methyl]oxetan-3-yl}pyridin-2-yl)amino]propanenitrile (350 mg, 1.05 mmol, 1 equiv) in dioxane (8 mL) was added Xantphos (120 mg, 0.20 mmol, 0.20 equiv), Pd(OAc)₂ (25 mg, 0.11 mmol, 0.11 equiv) and Cs₂CO₃ (860 mg, 2.64 mmol, 2.51 equiv) in portions at room temperature under nitrogen atmosphere. The resulting mixture was stirred for 1 h at 90° C. under nitrogen atmosphere. LCMS indicated the reaction was completed. The resulting mixture was filtered, the filter cake was washed with ACN and DCM. The filtrate was concentrated under reduced pressure. The residue was purified by silica gel column chromatography, eluted with CH₂Cl₂/MeOH (5:1) to afford 3-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]propanenitrile; formic acid (330 mg, 43%) as a green solid. LCMS: (ES, m/s): 609 [M+H]⁺

### Step 3: Synthesis of Compound 4

A mixture of 3-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]propanenitrile (950 mg, 1.56 mmol, 1 equiv), Boc₂O (1.36 g, 6.24 mmol, 4 equiv), TEA (790 mg, 7.80 mmol, 5 equiv) and DMAP (95 mg, 0.78 mmol, 0.5 equiv) in THE (15 mL) was stirred for 3 h at 50° C. LCMS indicated the reaction was completed. The solvent was removed under reduced pressure. The residue was purified by reverse flash chromatography with the following conditions: column, C18 silica gel, 80 g, 20-35 um; mobile phase, water with 0.1% TFA and ACN (0% to 100% gradient in 50 min); detector, UV 254 nm. The collected fraction was lyophilized to afford tert-butyl N-(2-cyanoethyl)-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (600 mg, 54%) as a white solid. LCMS (ESI, m/z): 709 [M+H]⁺.

### Step 4: Synthesis of Compound 5

To a mixture of tert-butyl N-(2-cyanoethyl)-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (550 mg, 0.77 mmol, 1 equiv) and KI (260 mg, 1.55 mmol, 2 equiv) in DMF (5 mL) was added 4-{[4-(chloromethyl)phenyl]sulfamoyl}-N-[2-(2,5-dioxopyrrol-1-yl)ethyl]-3-nitrobenzamide (380 mg, 0.77 mmol, 1 equiv) in DMF (2 mL) at 0° C. The resulting mixture was stirred for 16 h at 25° C. LCMS indicated the reaction was completed. The reaction system was purified by reverse flash chromatography with the following conditions: column, C18 silica gel, 80 g, 20-35 um; mobile phase, water with 0.1% TFA and ACN (0% to 100% gradient in 50 min); detector, UV 254 nm. The collected fraction was lyophilized to afford (3S)-1-[(2-{6-[(tert-butoxycarbonyl)(2-cyanoethyl)amino]-4-{3-[(4-methyl-1,2,4-triazol-3-yl)methyl]oxetan-3-yl}pyridin-2-yl}-3-oxo-7-(trifluoromethyl)-1H-isoindol-5-yl)methyl]-1-{[4-(4-{[2-(2,5-dioxopyrrol-1-yl)ethyl]carbamoyl}-2-nitrobenzenesulfonamido)phenyl]methyl}-3-methylpiperidin-1-ium (200 mg, 22%) as a yellow solid. LCMS (ESI, m/z): 1165 [M+H]⁺.

### Step 5: Synthesis of Compound 217

A mixture of (3S)-1-[(2-{6-[(tert-butoxycarbonyl)(2-cyanoethyl)amino]-4-{3-[(4-methyl-1,2,4-triazol-3-yl)methyl]oxetan-3-yl}pyridin-2-yl}-3-oxo-7-(trifluoromethyl)-1H-isoindol-5-yl)methyl]-1-{[4-(4-{[2-(2,5-dioxopyrrol-1-yl)ethyl]carbamoyl}-2-nitrobenzenesulfonamido)phenyl]methyl}-3-methylpiperidin-1-ium (190 mg, 0.16 mmol, 1 equiv) in DCM (7.5 mL) was added TFA (1.5 mL) at 25° C. The resulting mixture was stirred for 1 h at 25° C. LCMS indicated the reaction was completed. The solvent was removed under reduced pressure. The residue was purified by prep-HPLC with following conditions: Column: XSelect CSH Prep C18 OBD Column, 19*150 mm, 5 m; Mobile Phase A: Water (0.1% TFA), Mobile Phase B: ACN; Flow rate: 25 mL/min; Gradient: 18% B to 48% B in 7 min, 48% B; Wave Length: 254 nm; RT1 (min): 5; The collected was lyophilized to afford (3S)-1-[(2-{6-[(2-cyanoethyl)amino]-4-{3-[(4-methyl-1,2,4-triazol-3-yl)methyl]oxetan-3-yl}pyridin-2-yl}-3-oxo-7-(trifluoromethyl)-1H-isoindol-5-yl)methyl]-1-{[4-(4-{[2-(2,5-dioxopyrrol-1-yl)ethyl]carbamoyl}-2-nitrobenzenesulfonamido)phenyl]methyl}-3-methylpiperidin-1-ium; trifluoroacetic acid (84.9 mg, 42%) as an off-white solid. LCMS (ES, m/z): 1065 [M+H−TFA]⁺. ¹H NMR (400 MHz, DMSO-d₆) δ 11.06 (s, 1H), 10.04 (br s, 1H), 9.91 (s, 1H), 9.00 (t, J=6.0 Hz, 1H), 8.28-8.19 (m, 3H), 8.08 (s, 2H), 7.50 (s, 1H), 7.37-7.30 (m, 2H), 7.21-7.10 (m, 3H), 7.01 (s, 2H), 6.27 (s, 1H), 5.47 (s, 2H), 5.28 (s, 2H), 4.83-4.79 (m, 4H), 4.54 (s, 2H), 3.75 (s, 2H), 3.63-3.50 (m, 7H), 3.41-3.25 (m, 4H), 2.90-2.81 (m, 3H), 2.68-2.55 (m, 1H), 1.86-1.64 (m, 4H), 1.08-1.03 (m, 1H), 0.88 (d, J=6.4 Hz, 3H).

### Synthesis of Compound 218

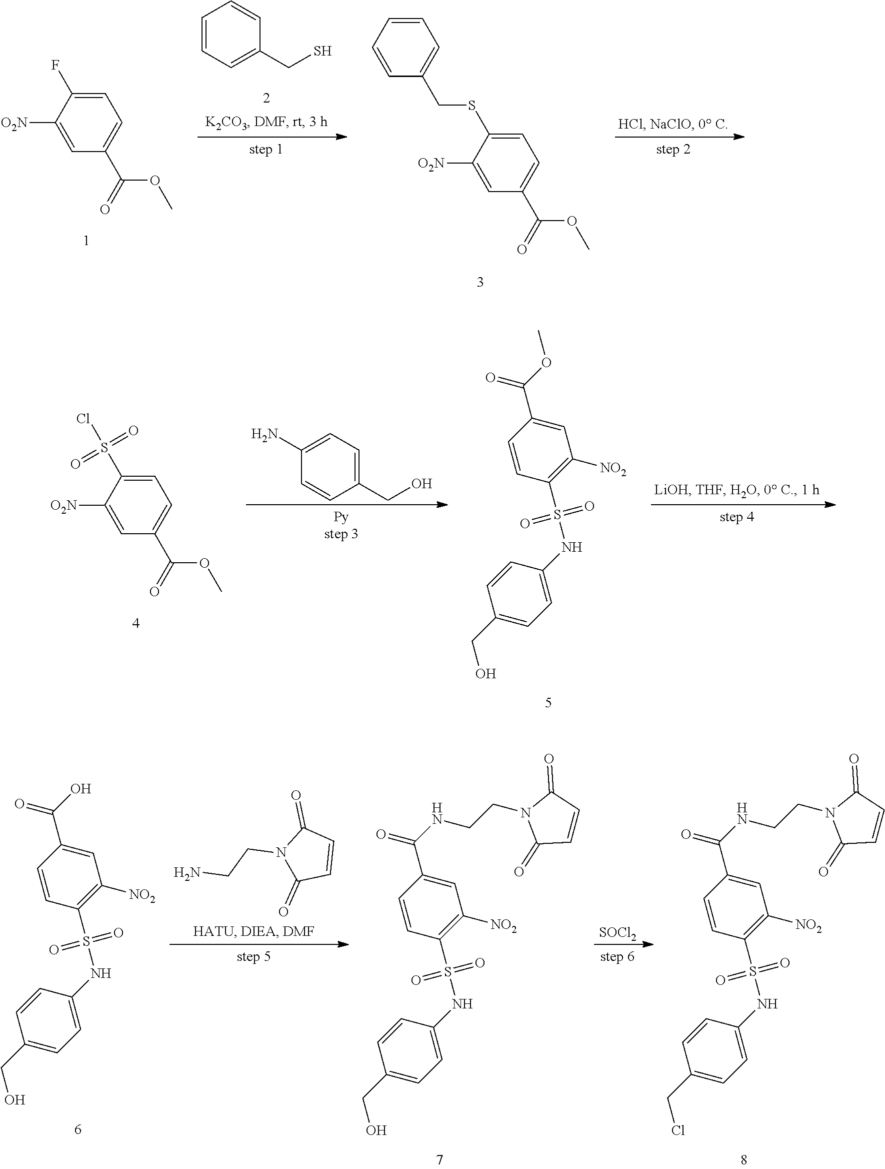

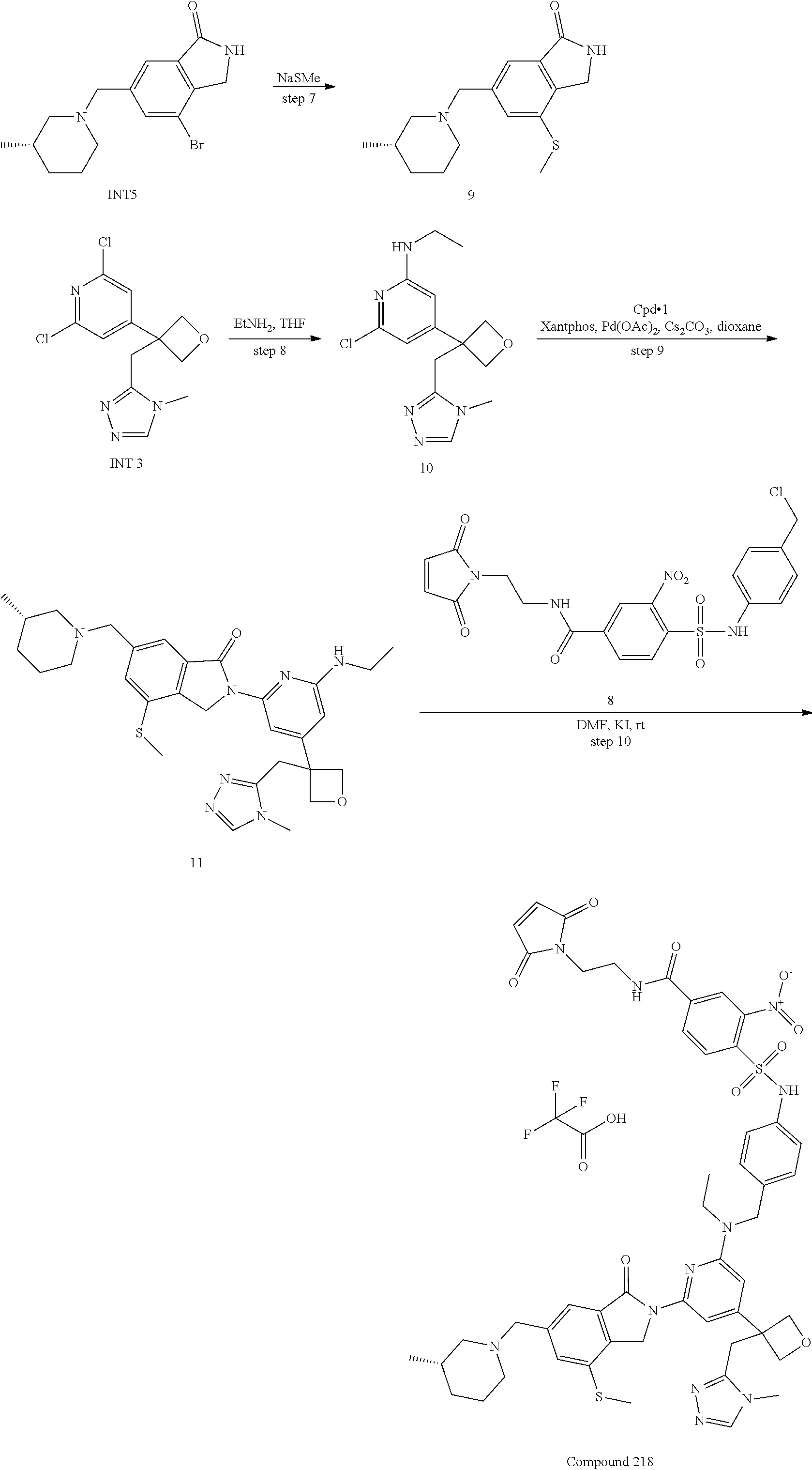

### Step 1: Synthesis of Compound 3

To a stirred mixture of methyl 4-fluoro-3-nitrobenzoate (25 g, 125.54 mmol, 1 equiv) and K₂CO₃ (34.70 g, 251.08 mmol, 2 equiv) in DMF (200 mL) was added benzyl mercaptan (31.18 g, 251.08 mmol, 2 equiv) in portions at 0° C. under nitrogen atmosphere. The resulting mixture was stirred for 3 h at room temperature under nitrogen atmosphere. LCMS indicated the reaction was completed. The reaction was quenched by the addition of Water/Ice (500 mL) at 0° C. The precipitated solids were collected by filtration and washed with PE (3×300 mL). The resulting solid was dried under infrared light. This resulted in methyl 4-(benzylsulfanyl)-3-nitrobenzoate (34 g, 89%) as a green solid. LCMS (ES, m/z): 304 [M+H]⁺

### Step 2: Synthesis of Compound 4

To a stirred solution of methyl 4-(benzylsulfanyl)-3-nitrobenzoate (25 g, 82.42 mmol, 1 equiv) in DCM (500 mL) was added HCl (aq, 6N, 500 mL) and NaClO (13%, 250 mL) in portions at 0° C. under nitrogen atmosphere. The resulting mixture was stirred for 1 h at 0° C. under nitrogen atmosphere. LCMS indicated the reaction was completed. The reaction was quenched with Water/Ice at 0° C. The resulting mixture was extracted with CH₂Cl₂ (3×500 mL). The combined organic layers were washed with brine (3×500 mL), dried over anhydrous Na₂SO₄. After filtration, the filtrate was concentrated under reduced pressure. The residue was purified by silica gel column chromatography, eluted with PE/EA (1:1) to afford methyl 4-(chlorosulfonyl)-3-nitrobenzoate (10 g, 43%) as a yellow solid. LCMS (ES, m/z): 260 [M−H-H₂O]⁻

### Step 3: Synthesis of Compound 5

To a stirred solution of β-aminobenzylalcohol (5.28 g, 42.91 mmol, 1.2 equiv) in DCM (100 mL) was added pyridine (5.66 g, 71.52 mmol, 2 equiv) and methyl 4-(chlorosulfonyl)-3-nitrobenzoate (10 g, 35.76 mmol, 1 equiv) dropwise at 0° C. under nitrogen atmosphere. The resulting mixture was stirred for 30 min at 0° C. under nitrogen atmosphere. LCMS indicated the reaction was completed. The reaction was quenched with Water/Ice at 0° C. The resulting mixture was extracted with CH₂Cl₂ (3×100 mL). The combined organic layers were washed with brine (3×100 mL), dried over anhydrous Na₂SO₄. After filtration, the filtrate was concentrated under reduced pressure. This resulted in methyl 4-{[4-(hydroxymethyl)phenyl]sulfamoyl}-3-nitrobenzoate (9 g, 68%) as a red solid. The crude product was used in the next step directly without further purification. LCMS (ES, m/z): 349 [M+H−H2O]

### Step 4: Synthesis of Compound 6

To a stirred solution of methyl 4-{[4-(hydroxymethyl)phenyl]sulfamoyl}-3-nitrobenzoate (7.5 g, 20.47 mmol, 1 equiv) in THE (750 mL) was added LiGH (0.98 g, 40.94 mmol, 2 equiv) in portions at 0° C. under nitrogen atmosphere. The resulting mixture was stirred for 1 h at 0° C. under nitrogen atmosphere. LCMS indicated the reaction was completed. The mixture was acidified to pH=3 with conc. HCl. The resulting mixture was purified by reversed-phase flash chromatography with the following conditions: column, C18 silica gel; mobile phase, ACN in Water (0.05% TFA), 5% to 60% gradient in 10 min; detector, UV 254 nm. The collected fraction was concentrated to afford 4-{[4-(hydroxymethyl)phenyl]sulfamoyl}-3-nitrobenzoic acid (3 g, 42%) as a red solid. LCMS (ES, m/z): 335 [M+H−H₂O]⁺

### Step 5: Synthesis of Compound 7

To a stirred solution of 4-{[4-(hydroxymethyl)phenyl]sulfamoyl}-3-nitrobenzoic acid (1.5 g, 4.25 mmol, 1 equiv) in DMF (15 mL) was added HATU (2.43 g, 6.38 mmol, 1.5 equiv) dropwise at 0° C. under nitrogen atmosphere. The resulting mixture was stirred for 30 min at 0° C. under nitrogen atmosphere. To the above mixture was added 1-(2-aminoethyl)pyrrole-2,5-dione (0.72 g, 5.108 mmol, 1.2 equiv) and DIEA(1.64 g, 12.75 mmol, 3 equiv) dropwise at 0° C. The resulting mixture was stirred for additional 2 h at room temperature. LCMS indicated the reaction was completed. The residue was purified by reversed-phase flash chromatography with the following conditions: column, C18 silica gel; mobile phase, ACN in Water (0.05% TFA), 5% to 60% gradient in 30 min; detector, UV 254 nm. The collected fraction was concentrated to afford N-[2-(2,5-dioxopyrrol-1-yl)ethyl]-4-{[4-(hydroxymethyl)phenyl]sulfamoyl}-3-nitrobenzamide (0.8 g, 40%) as a brown solid. LCMS (ES, m/z): 457 [M+H−H₂O]⁺

### Step 6: Synthesis of Compound 8

To a stirred mixture of N-[2-(2,5-dioxopyrrol-1-yl)ethyl]-4-{[4-(hydroxymethyl)phenyl]sulfamoyl}-3-nitrobenzamide (150 mg, 0.32 mmol, 1 equiv) and DMF (0.1 mL, 1.29 mmol, 4.09 equiv) in THE (3 mL) was added SOCl₂ (75 mg, 0.63 mmol, 2 equiv) in THE (0.05 mL) dropwise at 0° C. The resulting mixture was stirred at 0° C. for 30 min. LCMS indicated the reaction was completed. Then water (3 mL) was added into the above mixture and the resulting mixture was lyophilized to afford crude 4-{[4-(chloromethyl)phenyl]sulfamoyl}-N-[2-(2,5-dioxopyrrol-1-yl)ethyl]-3-nitrobenzamide (170 mg, crude) as a yellow solid. LCMS (ESI, m/z): 457 [M+H−HCl]⁺.

### Step 7: Synthesis of Compound 9

To a stirred solution of 4-bromo-6-{[(3S)-3-methylpiperidin-1-yl]methyl}-2,3-dihydroisoindol-1-one (200 mg, 0.61 mmol, 1 equiv) in DMF (3 mL) was added (methylsulfanyl)sodium (216 mg, 3.09 mmol, 5 equiv) dropwise at room temperature under air atmosphere. The resulting mixture was stirred for overnight at room temperature. LCMS indicated the reaction was completed. The mixture was allowed to cool down to room temperature. The reaction mixture was purified by reverse flash chromatography with the following conditions: column, C18 silica gel; mobile phase, ACN in water (0.05% TFA), 0% to 50% gradient in 40 min; detector, UV 254 nm. This resulted in 6-{[(3S)-3-methylpiperidin-1-yl]methyl}-4-(methylsulfanyl)-2,3-dihydroisoindol-1-one (80 mg, 44%) as a white solid. LCMS (ES, m/z): 291 [M+H]⁺

### Step 8: Synthesis of Compound 10

To a stirred solution of 2,6-dichloro-4-{3-[(4-methyl-1,2,4-triazol-3-yl)methyl]oxetan-3-yl}pyridine (800 mg, 2.67 mmol, 1 equiv) in Ethylamine solution (2.0 M in THF, 10 mL) was stirred for overnight at 90° C. under nitrogen atmosphere. LCMS indicated the reaction was completed. The mixture was allowed to cool down to room temperature and concentrated to dryness under vacuum. The residue was purified by silica gel column chromatography, eluted with CH₂Cl₂/MeOH (10:1) to afford 6-chloro-N-ethyl-4-{3-[(4-methyl-1,2,4-triazol-3-yl)methyl]oxetan-3-yl}pyridin-2-amine (450 mg, 54%) as an off-white solid. LCMS (ES, m/z): 308,310 [M+H]⁺

### Step 9: Synthesis of Compound 11

To a stirred mixture of 6-chloro-N-ethyl-4-{3-[(4-methyl-1,2,4-triazol-3-yl)methyl]oxetan-3-yl}pyridin-2-amine (65 mg, 0.21 mmol, 1 equiv) and 6-{[(3S)-3-methylpiperidin-1-yl]methyl}-4-(methylsulfanyl)-2,3-dihydroisoindol-1-one (61 mg, 0.21 mmol, 1 equiv) in dioxane (1 mL) was added Cs₂CO₃ (137 mg, 0.42 mmol, 2 equiv) in portions and Xantphos (24 mg, 0.04 mmol, 0.2 equiv) and Pd(OAc)₂ (5 mg, 0.02 mmol, 0.1 equiv) in portions at room temperature under nitrogen atmosphere. The resulting mixture was stirred for 2 h at 120° C. under nitrogen atmosphere. LCMS indicated the reaction was completed. The mixture was allowed to cool down to room temperature. The resulting mixture was filtered, the filter cake was washed with DCM (3×20 mL). The filtrate was concentrated under reduced pressure. The residue was purified by silica gel column chromatography, eluted with CH₂Cl₂/MeOH (10:1) to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (45 mg, crude). The crude product (45 mg) was re-purified by Prep-HPLC with the following conditions (Column: XBridge Prep Phenyl OBD Column, 19*250 mm, 5 m; Mobile Phase A: Water (0.05% TFA), Mobile Phase B: ACN; Flow rate: 25 mL/min; Gradient: 25% B to 55% B in 7 min, 55% B; Wave Length: 254 nm; RT1 (min): 5.5; The collected fraction was lyophilized to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (30.1 mg, 24%) as a yellow solid. LCMS (ES, m/z): 562 [M+H]^{+ 1}H NMR (400 MHz, DMSO-d₆) δ 9.66 (br s, 1H), 8.61 (s, 1H), 7.75-7.68 (m, 2H), 7.37 (s, 1H), 5.96 (s, 1H), 4.90-4.88 (m, 4H), 4.80-4.78 (m, 2H), 4.43-4.42 (m, 2H), 3.57 (s, 2H), 3.38-3.32 (m, 1H), 3.30-3.21 (m, 6H), 2.84-2.81 (m, 1H), 2.64-2.51 (m, 4H), 1.86-1.63 (m, 4H), 1.16-1.04 (m, 4H), 0.94-0.88 (m, 3H)

### Step 10: Synthesis of Compound 218

To a mixture of 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (100 mg, 0.178 mmol, 1 equiv) and KI (59 mg, 0.356 mmol, 2 equiv) in DMF (1 mL) was added 4-{[4-(chloromethyl)phenyl]sulfamoyl}-N-[2-(2,5-dioxopyrrol-1-yl)ethyl]-3-nitrobenzamide (87 mg, 0.178 mmol, 1 equiv) in DMF (1 mL) dropwise at 0° C. The resulting mixture was stirred for 16 h at 25° C. LCMS indicated the reaction was completed. The reaction was purified with prep-HPLC with following conditions: Column: XBridge Prep OBD C18 Column, 30*150 mm, 5 m; Mobile Phase A: Water (0.1% TFA), Mobile Phase B: ACN; Flow rate: 60 mL/min; Gradient: 27% B to 57% B in 7 min, 57% B; Wave Length: 254 nm; RT1 (min): 5; Number Of Runs: 0. The collected fraction was lyophilized. This batch was combined with next batch to afford N-[2-(2,5-dioxopyrrol-1-yl)ethyl]-4-[(4-{[ethyl(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)amino]methyl}phenyl)sulfamoyl]-3-nitrobenzamide (14.1 mg, 15%) as a yellow solid. LCMS (ESI, m/z): 1018 [M+H]⁺. ¹H NMR (400 MHz, DMSO-d₆) δ 10.78 (s, 1H), 9.65 (br s, 1H), 8.96 (t, J=6.0 Hz, 1H), 8.37 (s, 1H), 8.15 (s, 1H), 8.06-7.98 (m, 2H), 7.73-7.65 (m, 2H), 7.42 (s, 1H), 7.19-7.17 (m, 2H), 7.11-7.07 (m, 2H), 7.00 (s, 2H), 6.01 (s, 1H), 4.88 (d, J=6.0 Hz, 2H), 4.78 (d, J=6.0 Hz, 2H), 4.71 (s, 2H), 4.60 (s, 2H), 4.41 (d, J=8.0 Hz, 2H), 3.58-3.49 (m, 4H), 3.47-3.32 (m, 5H), 3.31-3.24 (m, 1H), 3.17 (s, 3H), 2.87-2.84 (m, 1H), 2.61-2.56 (m, 4H), 1.86-1.63 (m, 4H), 1.07-1.00 (m, 4H), 0.88 (d, J=6.5 Hz, 3H).

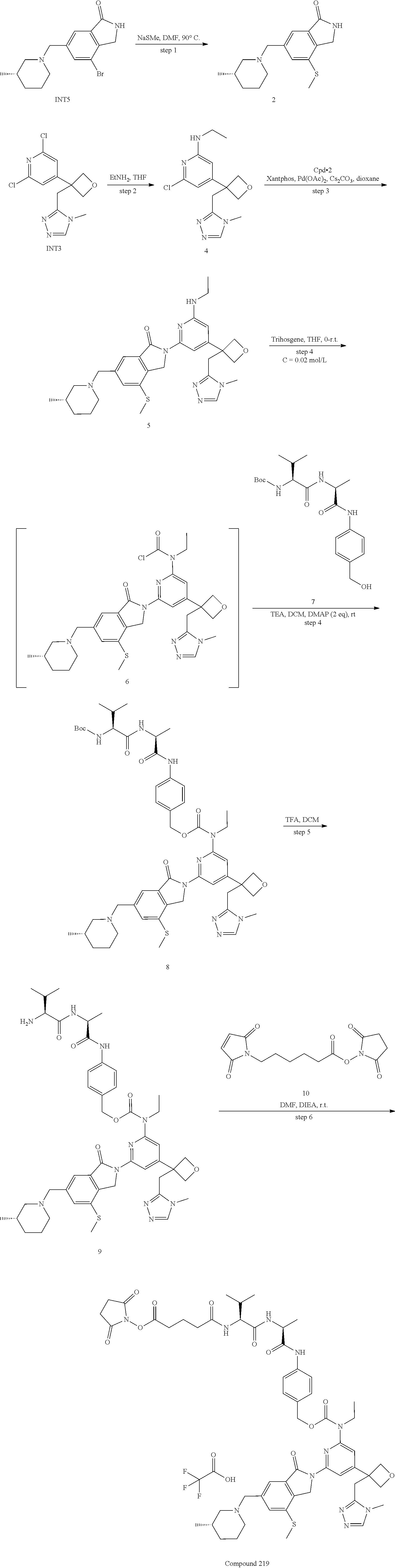

### Step 1. Synthesis of Compound 2

To a stirred solution of 4-bromo-6-{[(3S)-3-methylpiperidin-1-yl]methyl}-2,3-dihydroisoindol-1-one (200 mg, 0.62 mmol, 1 equiv) in DMF (3 mL) was added (methylsulfanyl)sodium (93 mg, 0.93 mmol, 1.5 equiv) at 25° C. The resulting mixture was stirred for 1 h at 90° C. LCMS indicated the reaction was completed. The reaction was purified by reverse flash chromatography with the following conditions: column, C18 silica gel; mobile phase, TFA in water, 0% to 50% gradient in 40 min; detector, UV 254 nm. This resulted in 6-{[(3S)-3-methylpiperidin-1-yl]methyl}-4-(methylsulfanyl)-2,3-dihydroisoindol-1-one (80 mg, 44%) as a white solid. LCMS:(ES·m/z): 291 [M+H]⁺.

### Step 2: Synthesis of Compound 4

To a stirred solution of 2,6-dichloro-4-{3-[(4-methyl-1,2,4-triazol-3-yl)methyl]oxetan-3-yl}pyridine (1.3 g, 4.34 mmol, 1 equiv) and in ethylamine solution (2.0 M in THF, 13 mL) was stirred for overnight at 90° C. LCMS indicated the reaction was completed. The mixture was allowed to cool down to room temperature. The resulting mixture was concentrated under vacuum. The residue was purified by silica gel column chromatography, eluted with CH₂Cl₂/MeOH (10:1) to afford 6-chloro-N-ethyl-4-{3-[(4-methyl-1,2,4-triazol-3-yl)methyl]oxetan-3-yl}pyridin-2-amine (440 mg, 33%) as a yellow solid. LCMS (ESI, ms): 308,310 [M+H]⁺.

### Step 3: Synthesis of Compound 5

To a stirred mixture of 6-{[(3S)-3-methylpiperidin-1-yl]methyl}-4-(methylsulfanyl)-2,3-dihydroisoindol-1-one (238 mg, 0.82 mmol, 1.0 equiv) and 6-chloro-N-ethyl-4-{3-[(4-methyl-1,2,4-triazol-3-yl)methyl]oxetan-3-yl}pyridin-2-amine (250 mg, 0.82 mmol, 1.0 equiv) in dioxane (4 mL) was added Xantphos (94 mg, 0.16 mmol, 0.2 equiv), Cs₂CO₃ (530 mg, 1.64 mmol, 2 equiv) and Pd(OAc)₂ (16 mg, 0.082 mmol, 0.1 equiv) at 25° C. The resulting mixture was stirred for 2 h at 120° C. under nitrogen atmosphere. LCMS indicated the reaction was completed. The solvent was removed under reduced pressure. The residue was purified by reverse flash chromatography with the following conditions: column, C18 silica gel, 80 g, 20-35 um; mobile phase, water with 0.1% TFA and ACN (5% to 95% gradient in 50 min); detector, UV 254 nm. The eluent was lyophilized to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (230 mg, 50%) as a yellow solid. LCMS (ESI, m/z): 562 [M+H]⁺.

### Step 4: Synthesis of Compound 8

To a stirred solution of 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (900 mg, 1.60 mmol, 1 equiv) in THE (80 mL) were added Triphosgene (713 mg, 2.40 mmol, 1.5 equiv) in portions at room temperature under nitrogen atmosphere. The resulting mixture was stirred for additional 30 min at room temperature. The resulting mixture was concentrated under vacuum. To the above mixture was added tert-butyl N-[(1S)-1-{[(1S)-1-{[4-(hydroxymethyl)phenyl]carbamoyl}ethyl]carbamoyl}-2-methylpropyl]carbamate (630 mg, 1.60 mmol, 1 equiv) in DCM (20 mL) were added TEA (486.37 mg, 4.806 mmol, 3 equiv) at 0° C. The resulting mixture was stirred for additional 10 min at 0° C. Finally add DMAP (391 mg, 3.20 mmol, 2 equiv) in portion. The resulting mixture was stirred for overnight at room temperature under nitrogen atmosphere. ˜10% desire product could be detected by LCMS. The resulting mixture was concentrated under reduced pressure. The residue was purified by reverse flash chromatography with the following conditions: column, C18 silica gel; mobile phase, MeCN in Water (0.1% TFA), 10% to 50% gradient in 10 min; detector, UV 254 nm. This resulted in {4-[(2S)-2-[(2S)-2-[(tert-butoxycarbonyl)amino]-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (180 mg, 10%) as a white solid. LCMS: (ES, m/s): 981 [M+H]⁺.

### Step 5: Synthesis of Compound 9

A mixture of {4-[(2S)-2-[(2S)-2-[(tert-butoxycarbonyl)amino]-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (180 mg, 0.18 mmol, 1 equiv) in DCM (7.5 mL) was added TFA (1.5 mL) at 25° C. The resulting mixture was stirred for 1 h at 25° C. LCMS indicated the reaction was completed. The solvent was removed under reduced pressure. The residue was lyophilized to afford crude {4-[(2S)-2-[(2S)-2-amino-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (180 mg, crude) as a brown solid. LCMS (ESI, m/z): 881 [M+H]⁺

### Step 6. Synthesis of Compound 219

To a stirred mixture of {4-[(2S)-2-[(2S)-2-amino-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (170 mg, 0.19 mmol, 1 equiv) and bis(2,5-dioxopyrrolidin-1-yl) pentanedioate (75 mg, 0.23 mmol, 1.19 equiv) in DMF (1 mL) was added DIEA (100 mg, 0.77 mmol, 4.01 equiv) dropwise at 0° C. under nitrogen atmosphere. The resulting mixture was stirred for 3 h at room temperature under nitrogen atmosphere. LCMS indicated the reaction was completed. The reaction mixture was purified by Prep-HPLC with the following conditions (Column: XBridge Prep OBD C18 Column, 19*250 mm, 5 m; Mobile Phase A: Water (0.1% FA), Mobile Phase B: ACN; Flow rate: 25 mL/min; Gradient: 35% B to 65% B in 10 min, 65% B; Wave Length: 254 nm; RT1 (min): 8.2; The collected fraction was lyophilized to afford 2,5-dioxopyrrolidin-1-yl 4-{[(1S)-1-{[(1S)-1-{[4-({[ethyl(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamoyl]oxy}methyl)phenyl]carbamoyl}ethyl]carbamoyl}-2-methylpropyl]carbamoyl}butanoate; trifluoroacetic acid (11 mg, 4%) as a white solid. LCMS:(ms, ESI) 1092[M+H−TFA]^{+ 1}H NMR (400 MHz, DMSO-d₆) δ 10.00 (s, 1H), 8.32-8.18 (m, 2H), 7.96 (s, 1H), 7.89-7.87 (m, 1H), 7.62 (d, J=8.2 Hz, 2H), 7.48 (d, J=5.2 Hz, 2H), 7.38 (d, J=8.2 Hz, 2H), 7.19 (s, 1H), 5.14 (s, 2H), 4.93-4.76 (m, 6H), 4.40-4.38 (m, 2H), 4.19-4.15 (m, 2H), 3.92-3.90 (m, 2H), 3.19 (s, 3H), 2.82 (s, 1H), 2.78-2.66 (m, 3H), 2.59-2.56 (m, 6H), 2.37-2.09 (m, 4H), 2.04-1.82 (m, 3H), 1.78-1.42 (m, 7H), 1.31 (d, J=7.2 Hz, 3H), 1.22 (t, J=6.8 Hz, 3H), 0.93-0.75 (m, 10H).

### Synthesis of Compound 220

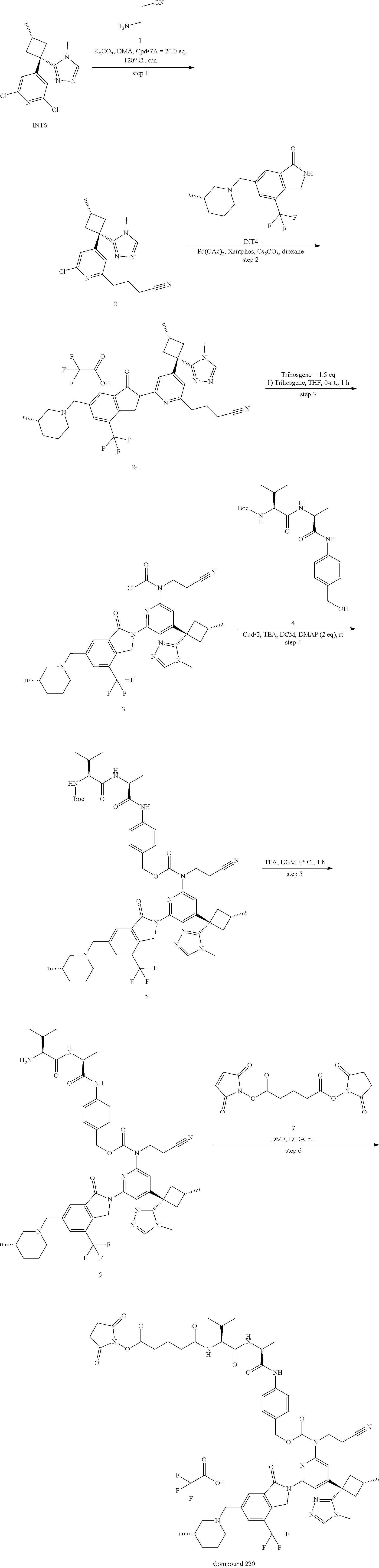

### Step 1: Synthesis of Compound 2

To a stirred solution of 2,6-dichloro-4-[(1r,3s)-3-methyl-1-(4-methyl-1,2,4-triazol-3-yl)cyclobutyl]pyridine (5 g, 16.82 mmol, 1 equiv) in DMA (50 mL) was added K₂CO₃ (4.65 g, 33.65 mmol, 2 equiv) and β-aminopropionitrile (23.59 g, 336.50 mmol, 20 equiv) dropwise at room temperature under nitrogen atmosphere. The resulting mixture was stirred for overnight at 120° C. under nitrogen atmosphere. The mixture was allowed to cool down to room temperature. LCMS indicated the reaction was completed. The reaction mixture was purified by reversed-phase flash chromatography with the following conditions: column, C18 silica gel; mobile phase, MeCN in Water (0.05% TFA), 0% to 50% gradient in 30 min; detector, UV 254 nm. The collected fraction was concentrated to afford 3-({6-chloro-4-[(1r,3s)-3-methyl-1-(4-methyl-1,2,4-triazol-3-yl)cyclobutyl]pyridin-2-yl}amino)propanenitrile (3 g, 54%) as a yellow solid. LCMS:(ESI·m/z):331,333[M+H]⁺.

### Step 2: Synthesis of Compound 2-1

To a stirred mixture of 3-({6-chloro-4-[(1r,3s)-3-methyl-1-(4-methyl-1,2,4-triazol-3-yl)cyclobutyl]pyridin-2-yl}amino)propanenitrile (2.5 g, 7.55 mmol, 1 equiv) and 6-{[(3S)-3-methylpiperidin-1-yl]methyl}-4-(trifluoromethyl)-2,3-dihydroisoindol-1-one (2.60 g, 8.31 mmol, 1.1 equiv) in dioxane (50 mL) was added Cs₂CO₃ (4.92 g, 15.11 mmol, 2 equiv) and Pd(OAc)₂ (0.17 g, 0.75 mmol, 0.1 equiv) and Xantphos (0.87 g, 1.51 mmol, 0.2 equiv) in portions at room temperature under nitrogen atmosphere. The resulting mixture was stirred for 1.5 h at 100° C. under nitrogen atmosphere. The mixture was allowed to cool down to room temperature. LCMS indicated the reaction was completed. The resulting mixture was concentrated under reduced pressure. The residue was purified by silica gel column chromatography, eluted with CH₂Cl₂/MeOH (10:1) to afford 3-{[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]amino}propanenitrile (1.3 g, 28%) as a yellow solid. LCMS:(ESI·m/z):607[M+H]⁺.

### Step 3: Synthesis of Compound 3

To a stirred solution of 3-{[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]amino}propanenitrile (400 mg, 0.66 mmol, 1 equiv) in THE (45 mL) was added Triphosgene (293 mg, 0.99 mmol, 1.5 equiv) in portions at room temperature under nitrogen atmosphere. The resulting mixture was stirred for 1 h at room temperature under nitrogen atmosphere. LCMS indicated the reaction was completed. The resulting mixture was concentrated under reduced pressure to afford N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamoyl chloride (420 mg) as a yellow solid. The crude product was used in the next step directly without further purification. LCMS:(ESI·m/z):669[M+H]⁺.

### Step 4: Synthesis of Compound 5

To a stirred mixture of N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamoyl chloride (400 mg, 0.598 mmol, 1 equiv) and tert-butyl N-[(1 S)-1-{[(1 S)-1-{[4-(hydroxymethyl)phenyl]carbamoyl}ethyl]carbamoyl}-2-methylpropyl]carbamate (258 mg, 0.65 mmol, 1.1 equiv) in DCM (10 mL) was added TEA (181 mg, 1.79 mmol, 3 equiv) dropwise at room temperature under air atmosphere. The resulting mixture was stirred for 30 min at room temperature under air atmosphere. To the above mixture was added N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamoyl chloride (420 mg, crude) and DMAP (146 mg, 1.19 mmol, 2 equiv) in portions at room temperature. The resulting mixture was stirred for additional overnight at room temperature. LCMS indicated the reaction was completed. The resulting mixture was concentrated under reduced pressure. The residue was purified by reverse flash chromatography with the following conditions: column, C18 silica gel; mobile phase, TFA in Water (0.05% TFA), 0% to 60% gradient in 30 min; detector, UV 254 nm. The collected fraction was concentrated to afford {4-[(2S)-2-[(2S)-2-[(tert-butoxycarbonyl)amino]-3-methylbutanamido]propanamido]phenyl}methyl N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamate (300 mg, two step 48%) as a white solid. LCMS:(ESI·m/z):1026[M+H]⁺, 514[M+H]/2.

### Step 5: Synthesis of Compound 6

To a stirred solution of {4-[(2S)-2-[(2S)-2-[(tert-butoxycarbonyl)amino]-3-methylbutanamido]propanamido]phenyl}methyl N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamate (340 mg, 0.33 mmol, 1 equiv) in DCM (7 mL) was added TFA (1.5 mL) dropwise at 0° C. under air atmosphere. The resulting mixture was stirred for 1 h at 0° C. under air atmosphere. LCMS indicated the reaction was completed. The resulting mixture was concentrated under reduced pressure. This resulted in {4-[(2S)-2-[(2S)-2-amino-3-methylbutanamido]propanamido]phenyl}methyl N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamate (300 mg, 97%) as a yellow oil. The crude product was used in the next step directly without further purification. LCMS:(ESI·m/z):926[M+H]⁺.

### Step 6: Synthesis of Compound 220

To a stirred mixture of {4-[(2S)-2-[(2S)-2-amino-3-methylbutanamido]propanamido]phenyl}methyl N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamate (150 mg, 0.16 mmol, 1 equiv) and bis(2,5-dioxopyrrolidin-1-yl) pentanedioate (79 mg, 0.24 mmol, 1.5 equiv) in DMF (3 mL) was added DIEA (83 mg, 0.65 mmol, 4 equiv) dropwise at 0° C. under air atmosphere. The resulting mixture was stirred for 2 h at room temperature under air atmosphere. LCMS indicated the reaction was completed. The reaction mixture was purified by Prep-HPLC with the following conditions (Column: XBridge Prep OBD C18 Column, 19*250 mm, 5 m; Mobile Phase A: Water (0.1% TFA), Mobile Phase B: ACN; Flow rate: 25 mL/min; Gradient: 35% B to 65% B in 7 min, 65% B; Wave Length: 254 nm; RT1 (min): 4.93; The collected fraction was lyophilized to afford 2,5-dioxopyrrolidin-1-yl 4-{[(1S)-1-{[(1S)-1-{[4-({[(2-cyanoethyl)[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamoyl]oxy}methyl)phenyl]carbamoyl}ethyl]carbamoyl}-2-methylpropyl]carbamoyl}butanoate; trifluoroacetic acid (27 mg, 13%) as a white solid. LCMS:(ESI·m/z):1137.5[M+H−TFA]⁺, 569.5[M+H−TFA]/2. ¹H NMR (400 MHz, DMSO-d₆) δ 10.02 (s, 1H), 9.84 (br s, 1H), 8.53-8.48 (m, 1H), 8.28-8.15 (m, 4H), 7.92-7.90 (d, J=8 Hz, 1H), 7.64-7.61 (m, 3H), 7.41 (d, J=8 Hz, 2H), 5.35 (s, 2H), 5.17 (s, 2H), 4.53-4.50 (m, 2H), 4.40-4.37 (m, 1H), 4.30-4.19 (m, 3H), 3.50-3.26 (m, 2H), 3.19 (s, 3H), 3.01-2.98 (m, 2H), 2.81 (s, 5H), 2.74-2.51 (m, 6H), 2.49-2.42 (m, 1H), 2.32-2.29 (m, 3H), 2.02-1.90 (m, 1H), 1.87-1.57 (m, 6H), 1.31 (d, J=8 Hz, 3H), 1.06-1.05 (m, 4H), 0.88-0.83 (m, 9H).

### Synthesis of Compound 221

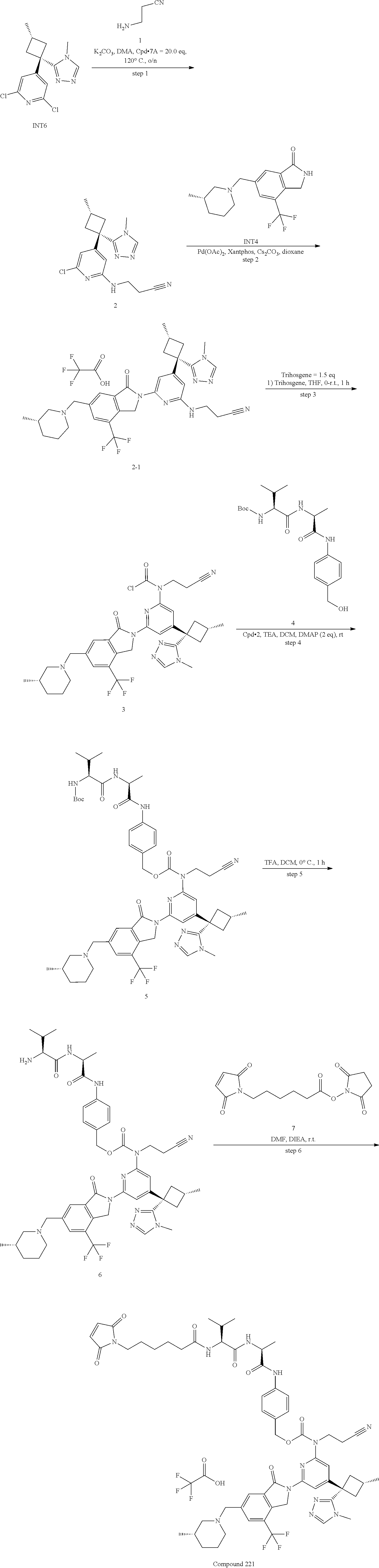

### Step 1: Synthesis of Compound 2

To a stirred solution of 2,6-dichloro-4-[(1r,3s)-3-methyl-1-(4-methyl-1,2,4-triazol-3-yl)cyclobutyl]pyridine (5 g, 16.82 mmol, 1 equiv) in DMA (50 mL) was added K₂CO₃ (4.65 g, 33.65 mmol, 2 equiv) and β-aminopropionitrile (23.59 g, 336.50 mmol, 20 equiv) dropwise at room temperature under nitrogen atmosphere. The resulting mixture was stirred for overnight at 120° C. under nitrogen atmosphere. The mixture was allowed to cool down to room temperature. LCMS indicated the reaction was completed. The reaction mixture was purified by reversed-phase flash chromatography with the following conditions: column, C18 silica gel; mobile phase, MeCN in Water (0.05% TFA), 0% to 50% gradient in 30 min; detector, UV 254 nm. The collected fraction was concentrated to afford 3-({6-chloro-4-[(1r,3s)-3-methyl-1-(4-methyl-1,2,4-triazol-3-yl)cyclobutyl]pyridin-2-yl}amino)propanenitrile (3 g, 54%) as a yellow solid. LCMS:(ESI·m/z):331,333[M+H]⁺.

### Step 2: Synthesis of Compound 2-1

To a stirred mixture of 3-({6-chloro-4-[(1r,3s)-3-methyl-1-(4-methyl-1,2,4-triazol-3-yl)cyclobutyl]pyridin-2-yl}amino)propanenitrile (2.5 g, 7.55 mmol, 1 equiv) and 6-{[(3S)-3-methylpiperidin-1-yl]methyl}-4-(trifluoromethyl)-2,3-dihydroisoindol-1-one (2.60 g, 8.31 mmol, 1.1 equiv) in dioxane (50 mL) was added Cs₂CO₃ (4.92 g, 15.11 mmol, 2 equiv) and Pd(OAc)₂ (0.17 g, 0.75 mmol, 0.1 equiv) and Xantphos (0.87 g, 1.51 mmol, 0.2 equiv) in portions at room temperature under nitrogen atmosphere. The resulting mixture was stirred for 1.5 h at 100° C. under nitrogen atmosphere. The mixture was allowed to cool down to room temperature. LCMS indicated the reaction was completed. The resulting mixture was concentrated under reduced pressure. The residue was purified by silica gel column chromatography, eluted with CH₂Cl₂/MeOH (10:1) to afford 3-{[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]amino}propanenitrile (1.3 g, 28%) as a yellow solid. LCMS:(ESI·m/z):607[M+H]⁺.

### Step 3: Synthesis of Compound 3

To a stirred solution of 3-{[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]amino}propanenitrile (400 mg, 0.66 mmol, 1 equiv) in THE (45 mL) was added Triphosgene (293 mg, 0.99 mmol, 1.5 equiv) in portions at room temperature under nitrogen atmosphere. The resulting mixture was stirred for 1 h at room temperature under nitrogen atmosphere. LCMS indicated the reaction was completed. The resulting mixture was concentrated under reduced pressure to afford N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamoyl chloride (420 mg) as a yellow solid. The crude product was used in the next step directly without further purification. LCMS:(ESI·m/z):669[M+H]⁺.

### Step 4: Synthesis of Compound 5

To a stirred mixture of tert-butyl N-[(1S)-1-{[(1S)-1-{[4-(hydroxymethyl)phenyl]carbamoyl}ethyl]carbamoyl}-2-methylpropyl]carbamate (258 mg, 0.65 mmol, 1.1 equiv) in DCM (10 mL) was added TEA (181 mg, 1.79 mmol, 3 equiv) dropwise at room temperature under air atmosphere. The resulting mixture was stirred for 30 min at room temperature under air atmosphere. To the above mixture was added N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamoyl chloride (420 mg, crude) and DMAP (146 mg, 1.19 mmol, 2 equiv) in portions at room temperature. The resulting mixture was stirred for additional overnight at room temperature. LCMS indicated the reaction was completed. The resulting mixture was concentrated under reduced pressure. The residue was purified by reverse flash chromatography with the following conditions: column, C18 silica gel; mobile phase, TFA in Water (0.05% TFA), 0% to 60% gradient in 30 min; detector, UV 254 nm. The collected fraction was concentrated to afford {4-[(2S)-2-[(2S)-2-[(tert-butoxycarbonyl)amino]-3-methylbutanamido]propanamido]phenyl}methyl N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamate (300 mg, two step 48%) as a white solid. LCMS:(ESI·m/z):1026[M+H]⁺, 514[M+H]/2.

### Step 5: Synthesis of Compound 6

To a stirred solution of {4-[(2S)-2-[(2S)-2-[(tert-butoxycarbonyl)amino]-3-methylbutanamido]propanamido]phenyl}methyl N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamate (340 mg, 0.33 mmol, 1 equiv) in DCM (7 mL) was added TFA (1.5 mL) dropwise at 0° C. under air atmosphere. The resulting mixture was stirred for 1 h at 0° C. under air atmosphere. LCMS indicated the reaction was completed. The resulting mixture was concentrated under reduced pressure. This resulted in {4-[(2S)-2-[(2S)-2-amino-3-methylbutanamido]propanamido]phenyl}methyl N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamate (300 mg, 97%) as a yellow oil. The crude product was used in the next step directly without further purification. LCMS:(ESI·m/z):926[M+H]⁺.

### Step 6: Synthesis of Compound 221

To a stirred mixture of {4-[(2S)-2-[(2S)-2-amino-3-methylbutanamido]propanamido]phenyl}methyl N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamate (150 mg, 0.16 mmol, 1 equiv) and 2,5-dioxopyrrolidin-1-yl 6-(2,5-dioxopyrrol-1-yl)hexanoate (75 mg, 0.24 mmol, 1.5 equiv) in DMF (3 mL) were added DIEA (84 mg, 0.64 mmol, 4 equiv) dropwise at 0° C. The resulting mixture was stirred for 2 h at room temperature under air atmosphere. LCMS indicated the reaction was completed. The reaction mixture was purified by Prep-HPLC with the following conditions (Column: XSelect CSH Prep C18 OBD Column, 19*250 mm, 5 m; Mobile Phase A: Water (0.1% TFA), Mobile Phase B: ACN; Flow rate: 25 mL/min; Gradient: 28% B to 50% B in 10 min, 50% B; Wave Length: 254 nm; RT1 (min): 7.97; The collected fraction was lyophilized to afford trifluoroacetic acid; {4-[(2S)-2-[(2S)-2-[6-(2,5-dioxopyrrol-1-yl)hexanamido]-3-methylbutanamido]propanamido]phenyl}methyl N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamate (60.1 mg, 29%) as a white solid. LCMS:(ESI·m/z):1119[M+H−TFA]⁺, 560[M+H−TFA]/2. ¹H NMR (400 MHz, DMSO-d₆) δ10.01 (m, 2H), 8.68 (m, 1H), 8.28-8.16 (m, 4H), 7.83-7.81 (d, J=8 Hz, 1H), 7.70-7.62 (m, 3H), 7.41-7.39 (m, 2H), 7.00 (s, 2H), 5.35 (s, 2H), 5.17 (s, 2H), 4.53 (s, 2H), 4.40-4.15 (m, 4H), 3.38-3.34 (m, 3H), 3.29-3.22 (m, 4H), 3.01-2.98 (m, 2H), 2.75-2.51 (m, 6H), 2.44-2.42 (m, 1H), 2.18-1.96 (m, 3H), 1.94-1.72 (m, 4H), 1.51-1.43 (m, 4H), 1.31-1.29 (m, 3H), 1.22-1.18 (m, 2H), 1.16-1.05 (m, 4H), 0.89-0.81 (m, 9H).

### Synthesis of Compound 222

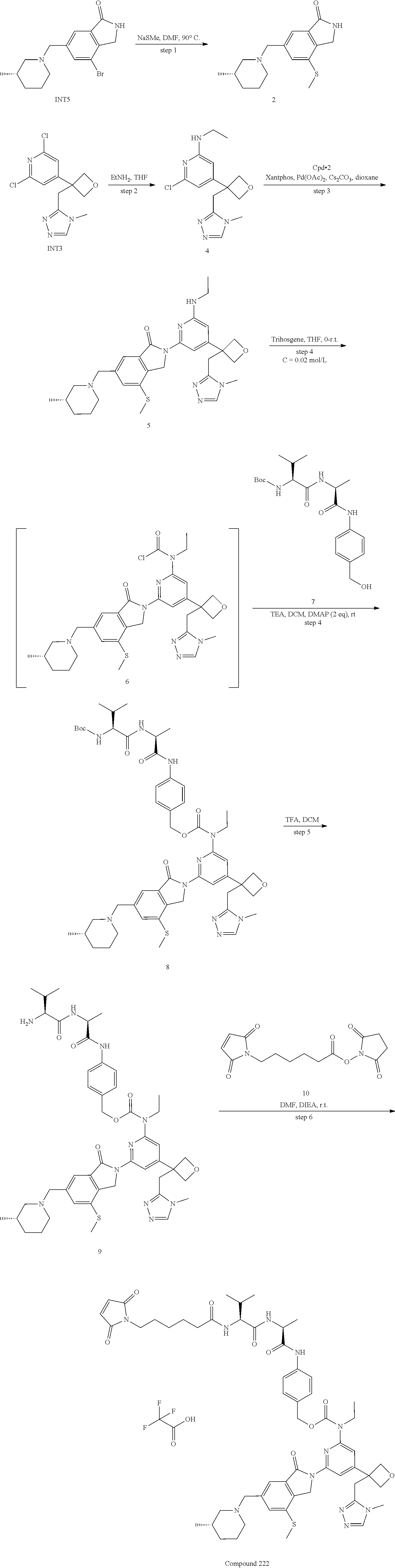

### Step 1: Synthesis of Compound 2

To a stirred solution of 4-bromo-6-{[(3S)-3-methylpiperidin-1-yl]methyl}-2,3-dihydroisoindol-1-one (200 mg, 0.62 mmol, 1 equiv) in DMF (3 mL) was added (methylsulfanyl)sodium (93 mg, 0.93 mmol, 1.5 equiv) at 25° C. The resulting mixture was stirred for 1 h at 90° C. LCMS indicated the reaction was completed. The reaction was purified by reverse flash chromatography with the following conditions: column, C18 silica gel; mobile phase, TFA in water, 0% to 50% gradient in 40 min; detector, UV 254 nm. This resulted in 6-{[(3S)-3-methylpiperidin-1-yl]methyl}-4-(methylsulfanyl)-2,3-dihydroisoindol-1-one (80 mg, 44%) as a white solid. LCMS:(ES·m/z): 291 [M+H]⁺.

### Step 2: Synthesis of Compound 4

To a stirred solution of 2,6-dichloro-4-{3-[(4-methyl-1,2,4-triazol-3-yl)methyl]oxetan-3-yl}pyridine (1.3 g, 4.34 mmol, 1 equiv) and in ethylamine solution (2.0 M in THF, 13 mL) was stirred for overnight at 90° C. LCMS indicated the reaction was completed. The mixture was allowed to cool down to room temperature. The resulting mixture was concentrated under vacuum. The residue was purified by silica gel column chromatography, eluted with CH₂Cl₂/MeOH (10:1) to afford 6-chloro-N-ethyl-4-{3-[(4-methyl-1,2,4-triazol-3-yl)methyl]oxetan-3-yl}pyridin-2-amine (440 mg, 33%) as a yellow solid. LCMS (ESI, ms): 308,310 [M+H]⁺.

### Step 3: Synthesis of Compound 5

To a stirred mixture of 6-{[(3S)-3-methylpiperidin-1-yl]methyl}-4-(methylsulfanyl)-2,3-dihydroisoindol-1-one (238 mg, 0.82 mmol, 1.0 equiv) and 6-chloro-N-ethyl-4-{3-[(4-methyl-1,2,4-triazol-3-yl)methyl]oxetan-3-yl}pyridin-2-amine (250 mg, 0.82 mmol, 1.0 equiv) in dioxane (4 mL) was added Xantphos (94 mg, 0.16 mmol, 0.2 equiv), Cs₂CO₃ (530 mg, 1.64 mmol, 2 equiv) and Pd(OAc)₂ (16 mg, 0.082 mmol, 0.1 equiv) at 25° C. The resulting mixture was stirred for 2 h at 120° C. under nitrogen atmosphere. LCMS indicated the reaction was completed. The solvent was removed under reduced pressure. The residue was purified by reverse flash chromatography with the following conditions: column, C18 silica gel, 80 g, 20-35 um; mobile phase, water with 0.1% TFA and ACN (5% to 95% gradient in 50 min); detector, UV 254 nm. The eluent was lyophilized to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (230 mg, 50%) as a yellow solid. LCMS (ESI, m/z): 562 [M+H]⁺.

### Step 4: Synthesis of Compound 8

To a stirred solution of 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (900 mg, 1.60 mmol, 1 equiv) in THF (80 mL) were added Triphosgene (713 mg, 2.40 mmol, 1.5 equiv) in portions at room temperature under nitrogen atmosphere. The resulting mixture was stirred for additional 30 min at room temperature. The resulting mixture was concentrated under vacuum. To the above mixture was added tert-butyl N-[(1S)-1-{[(1S)-1-{[4-(hydroxymethyl)phenyl]carbamoyl}ethyl]carbamoyl}-2-methylpropyl]carbamate (630 mg, 1.60 mmol, 1 equiv) in DCM (20 mL) were added TEA (486.37 mg, 4.806 mmol, 3 equiv) at 0° C. The resulting mixture was stirred for additional 10 min at 0° C. Finally add DMAP (391 mg, 3.20 mmol, 2 equiv) in portion. The resulting mixture was stirred for overnight at room temperature under nitrogen atmosphere. ˜10% desire product could be detected by LCMS. The resulting mixture was concentrated under reduced pressure. The residue was purified by reverse flash chromatography with the following conditions: column, C18 silica gel; mobile phase, MeCN in Water (0.1% TFA), 10% to 50% gradient in 10 min; detector, UV 254 nm. This resulted in {4-[(2S)-2-[(2S)-2-[(tert-butoxycarbonyl)amino]-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (180 mg, 10%) as a white solid. LCMS: (ES, m/s): 981 [M+H]⁺.

### Step 5: Synthesis of Compound 9

A mixture of {4-[(2S)-2-[(2S)-2-[(tert-butoxycarbonyl)amino]-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (180 mg, 0.18 mmol, 1 equiv) in DCM (7.5 mL) was added TFA (1.5 mL) at 25° C. The resulting mixture was stirred for 1 h at 25° C. LCMS indicated the reaction was completed. The solvent was removed under reduced pressure. The residue was lyophilized to afford crude {4-[(2S)-2-[(2S)-2-amino-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (180 mg, crude) as a brown solid. LCMS (ESI, m/z): 881 [M+H]⁺

### Step 6: Synthesis of Compound 222

A mixture of {4-[(2S)-2-[(2S)-2-amino-3-methylbutanamido]propanamido]phenyl}methyl N-ethyl-N-(4-{3-[(4-methyl-1,2,4-triazol-3-yl)methyl]oxetan-3-yl}-6-(6-{[(3 S)-3-methylpiperidin-1-yl]methyl}-4-(methylsulfanyl)-1-oxo-3H-isoindol-2-yl)pyridin-2-yl)carbamate (160 mg, 0.18 mmol, 1.0 equiv), DIEA (117 mg, 0.91 mmol, 5 equiv) and 2,5-dioxopyrrolidin-1-yl 6-(2,5-dioxopyrrol-1-yl)hexanoate (56 mg, 0.18 mmol, 1.0 equiv) in DMF (3 mL) was stirred for 1 h at 25° C. LCMS indicated the reaction was completed. The reaction mixture was purified by prep-HPLC with following conditions: Column: XSelect CSH Prep C18 OBD Column, 19*150 mm, 5 m; Mobile Phase A: Water (0.05% TFA), Mobile Phase B: ACN; Flow rate: 25 mL/min; Gradient: 16% B to 48% B in 12 min, 48% B; Wave Length: 254 nm; RT1 (min): 11.53; The collected fraction was lyophilized to afford trifluoroacetic acid; {4-[(2S)-2-[(2S)-2-[6-(2,5-dioxopyrrol-1-yl)hexanamido]-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (17.1 mg, 8%) as a white solid. LCMS (ESI, m/z): 1074 [M+H−TFA]^{+ 1}H NMR (400 MHz, Methanol-d4) δ 8.29 (s, 1H) 7.98 (s, 1H), 7.70 (s, 1H), 7.66-7.63 (m, 3H), 7.42-7.40 (m, 2H), 7.16 (s, 1H), 6.79 (s, 2H), 5.21 (s, 2H), 5.07-4.96 (m, 6H), 4.50-4.43 (m, 3H), 4.15 (d, J=7.2 Hz, 1H), 4.10-4.05 (m, 2H), 3.63 (s, 2H), 3.52-3.48 (m, 4H), 3.18 (s, 3H), 3.000-2.95 (m, 1H), 2.73-2.69 (m, 1H), 2.62 (s, 3H), 2.29 (t, J=7.3 Hz, 2H), 2.12-2.08 (m, 1H), 2.02-1.74 (m, 4H), 1.70-1.53 (m, 4H), 1.46 (d, J=7.1 Hz, 3H), 1.36-1.20 (m, 6H), 1.02-0.97 (m, 9H).

### Synthesis of Compound 223

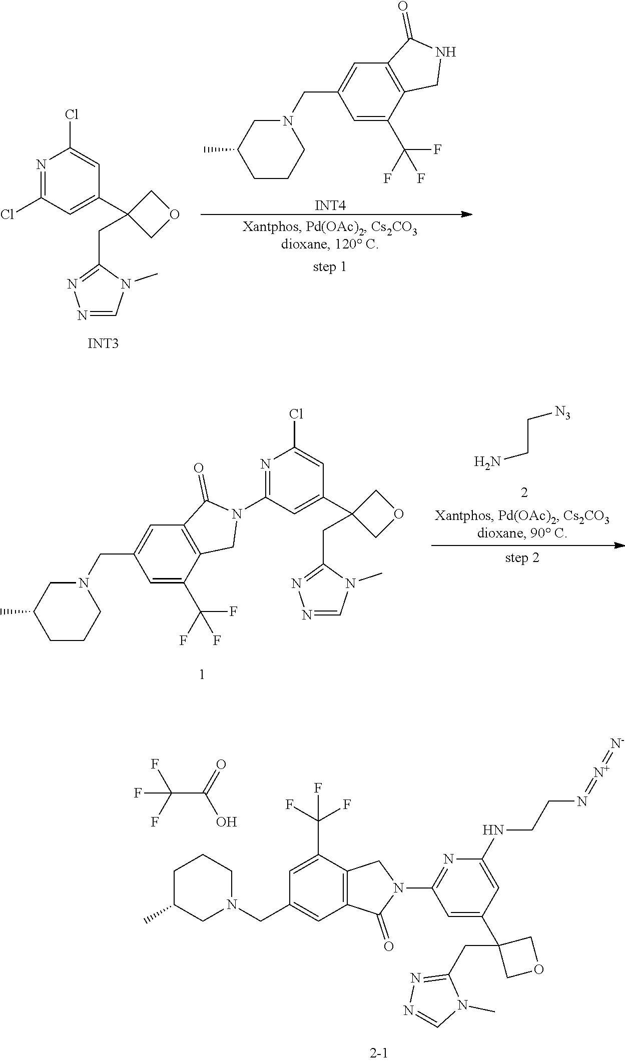

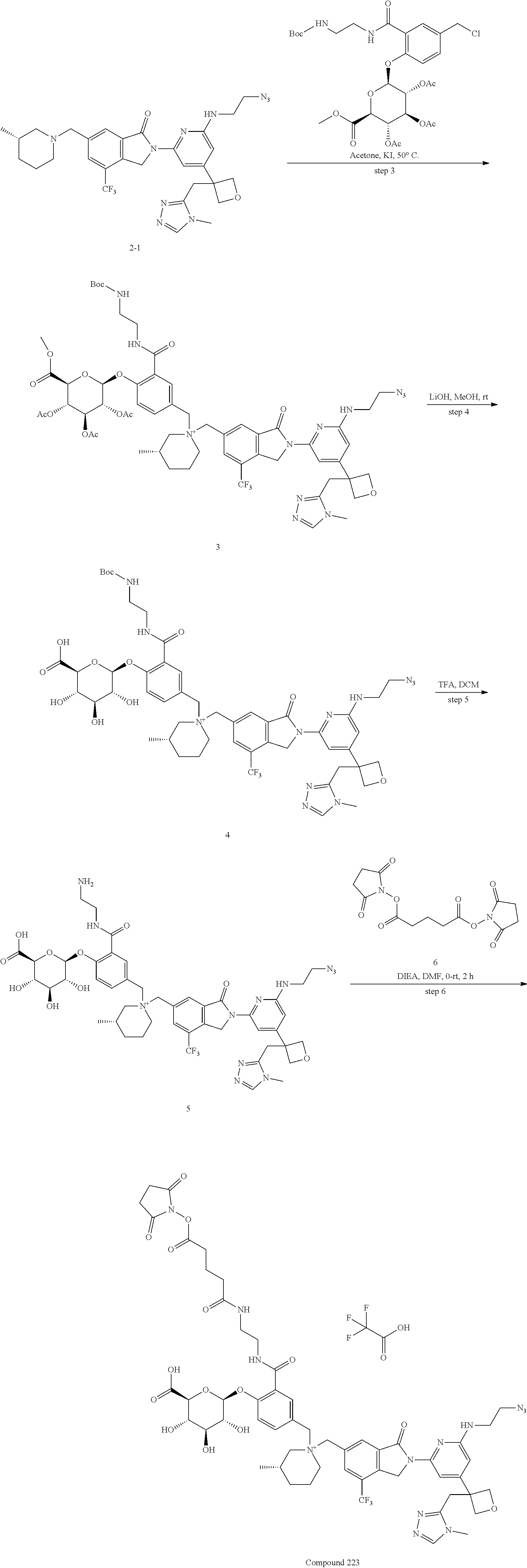

### Step 1: Synthesis of Compound 1

To a stirred mixture of 2,6-dichloro-4-{3-[(4-methyl-1,2,4-triazol-3-yl)methyl]oxetan-3-yl}pyridine (1.05 g, 3.52 mmol, 1.10 equiv) and 6-{[(3S)-3-methylpiperidin-1-yl]methyl}-4-(trifluoromethyl)-2,3-dihydroisoindol-1-one (1 g, 3.20 mmol, 1.00 equiv) in dioxane (10 mL) was added Cs₂CO₃ (2.09 g, 6.40 mmol, 2 equiv) and Xantphos (0.37 g, 0.64 mmol, 0.2 equiv) and Pd(OAc)₂ (112 mg, 0.50 mmol, 0.15 equiv) in portions at room temperature. The resulting mixture was stirred for 2 h at 120° C. under nitrogen atmosphere. The mixture was allowed to cool down to room temperature. LCMS indicated the reaction was completed. The resulting mixture was filtered, the filter cake was washed with CH₂Cl₂ (3×20 mL). The filtrate was concentrated under reduced pressure. The residue was purified by silica gel column chromatography, eluted with CH₂Cl₂/MeOH (10:1) to afford 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(trifluoromethyl)-3H-isoindol-1-one (630 mg, 29%) as a green solid. LCMS:(ES·m/z):575,577[M+H]⁺.

### Step 2: Synthesis of Compound 2-1

To a stirred mixture of 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(trifluoromethyl)-3H-isoindol-1-one (100 mg, 0.17 mmol, 1 equiv) and 2-azidoethanamine (22 mg, 0.26 mmol, 1.5 equiv) in dioxane (1 mL) was added Cs₂CO₃ (113 mg, 0.34 mmol, 2 equiv) and Xantphos (20 mg, 0.03 mmol, 0.2 equiv) and Pd(OAc)₂ (4 mg, 0.02 mmol, 0.1 equiv) in portions at room temperature. The resulting mixture was stirred for 1.5 h at 90° C. under nitrogen atmosphere. LCMS indicated 50% product on LCMS. The resulting mixture was filtered, the filter cake was washed with DCM (3×50 mL). The filtrate was concentrated under reduced pressure. The residue was purified by silica gel column chromatography, eluted with CH₂Cl₂/MeOH (10:1) to afford 2-{6-[(2-azidoethyl)amino]-4-{3-[(4-methyl-1,2,4-triazol-3-yl)methyl]oxetan-3-yl}pyridin-2-yl}-6-{[(3S)-3-methylpiperidin-1-yl]methyl}-4-(trifluoromethyl)-3H-isoindol-1-one (30 mg, crude). The crude product (30 mg) was purified by Prep-HPLC with the following conditions (Column: XSelect CSH Prep C18 OBD Column, 19*250 mm, 5 m; Mobile Phase A: Water (0.05% TFA), Mobile Phase B: ACN; Flow rate: 25 mL/min; Gradient: 20% B to 35% B in 10 min, 35% B; Wave Length: 254 nm; RT1 (min): 8.77; The collected fraction was lyophilize to afford 2-{6-[(2-azidoethyl)amino]-4-{3-[(4-methyl-1,2,4-triazol-3-yl)methyl]oxetan-3-yl}pyridin-2-yl}-6-{[(3S)-3-methylpiperidin-1-yl]methyl}-4-(trifluoromethyl)-3H-isoindol-1-one (8.5 mg, 8%) as a yellow solid. LCMS:(ES·m/z):625[M+H]⁺. ¹H NMR (400 MHz, DMSO-d₆) δ 9.81 (s, 1H), 8.45 (s, 1H), 8.27-8.19 (m, 2H), 7.44 (s, 1H), 6.98 (br s, 1H), 6.07 (s, 1H), 5.24 (s, 2H), 4.91-4.85 (m, 2H), 4.79-4.67 (m, 2H), 4.52 (s, 2H), 3.55 (s, 1H), 3.49-3.48 (m, 4H), 3.39-3.36 (m, 1H), 3.29-3.21 (m, 4H), 2.86-2.84 (m, 1H), 2.63-2.56 (m, 1H), 2.49-2.48 (m, 1H), 1.85-1.62 (m, 4H), 1.10-1.12 (m, 1H), 0.94 (d, J=5.6 Hz, 3H).

### Step 3: Synthesis of Compound 3

A mixture of 2-{6-[(2-azidoethyl)amino]-4-{3-[(4-methyl-1,2,4-triazol-3-yl)methyl]oxetan-3-yl}pyridin-2-yl}-6-{[(3S)-3-methylpiperidin-1-yl]methyl}-4-(trifluoromethyl)-3H-isoindol-1-one (100 mg, 0.16 mmol, 1.0 equiv), methyl (2S,3S,4S,5R,6S)-3,4,5-tris(acetyloxy)-6-[2-({2-[(tert-butoxycarbonyl)amino]ethyl}carbamoyl)-4-(chloromethyl)phenoxy]oxane-2-carboxylate (103 mg, 0.16 mmol, 1.0 equiv) and potassium iodide (53 mg, 0.32 mmol, 2.0 equiv) in acetone (2 mL) was stirred for 4 h at 50° C. LCMS indicated the reaction was completed. The reaction was purified by reverse flash chromatography with the following conditions: column, C18 silica gel, 80 g, 20-35 um; mobile phase, water with 0.1% TFA and ACN (0% to 100% gradient in 30 min); detector, UV 254 nm. The collected fraction was concentrated to afford (3S)-1-[(2-{6-[(2-azidoethyl)amino]-4-{3-[(4-methyl-1,2,4-triazol-3-yl)methyl]oxetan-3-yl}pyridin-2-yl}-3-oxo-7-(trifluoromethyl)-1H-isoindol-5-yl)methyl]-1-{[3-({2-[(tert-butoxycarbonyl)amino]ethyl}carbamoyl)-4-{[(2S,3R,4S,5S,6S)-3,4,5-tris(acetyloxy)-6-(methoxycarbonyl)oxan-2-yl]oxy}phenyl]methyl}-3-methylpiperidin-1-ium (95 mg, 48%) as a yellow solid. LCMS (ESI, m/z): 1233.50 [M+H]⁺.

### Step 4: Synthesis of Compound 4

To a mixture of (3S)-1-[(2-{6-[(2-azidoethyl)amino]-4-{3-[(4-methyl-1,2,4-triazol-3-yl)methyl]oxetan-3-yl}pyridin-2-yl}-3-oxo-7-(trifluoromethyl)-1H-isoindol-5-yl)methyl]-1-{[3-({2-[(tert-butoxycarbonyl)amino]ethyl}carbamoyl)-4-{[(2S,3R,4S,5S,6S)-3,4,5-tris(acetyloxy)-6-(methoxycarbonyl)oxan-2-yl]oxy}phenyl]methyl}-3-methylpiperidin-1-ium (90 mg, 0.073 mmol, 1.0 equiv) in MeOH (2 mL) was added lithium hydroxide (14 mg, 0.58 mmol, 8.0 equiv). The resulting mixture was stirred for 1 h at 25° C. LCMS indicated the reaction was completed. The reaction was concentrated to afford crude (3S)-1-[(2-{6-[(2-azidoethyl)amino]-4-{3-[(4-methyl-1,2,4-triazol-3-yl)methyl]oxetan-3-yl}pyridin-2-yl}-3-oxo-7-(trifluoromethyl)-1H-isoindol-5-yl)methyl]-1-{[3-({2-[(tert-butoxycarbonyl)amino]ethyl}carbamoyl)-4-{[(2S,3R,4S,5S,6S)-6-carboxy-3,4,5-trihydroxyoxan-2-yl]oxy}phenyl]methyl}-3-methylpiperidin-1-ium (125 mg, crude) as a white solid. LCMS (ESI, m/z): 1121.40 [M+H]⁺.

### Step 5: Synthesis of Compound 5

A mixture of crude (3S)-1-[(2-{6-[(2-azidoethyl)amino]-4-{3-[(4-methyl-1,2,4-triazol-3-yl)methyl]oxetan-3-yl}pyridin-2-yl}-3-oxo-7-(trifluoromethyl)-1H-isoindol-5-yl)methyl]-1-{[3-({2-[(tert-butoxycarbonyl)amino]ethyl}carbamoyl)-4-{[(2S,3R,4S,5S,6S)-6-carboxy-3,4,5-trihydroxyoxan-2-yl]oxy}phenyl]methyl}-3-methylpiperidin-1-ium (115 mg, 0.105 mmol, 1.0 equiv) in DCM (2.5 mL) was added TFA (0.5 mL) at 25° C. The resulting mixture was stirred for 1 h at 25° C. LCMS indicated the reaction was completed. The solvent was removed under reduced pressure. The residue was purified with following conditions: Column: XBridge Prep OBD C18 Column, 19*250 mm, 5 m; Mobile Phase A: Water (0.1% TFA), Mobile Phase B: ACN; Flow rate: 25 mL/min; Gradient: 12% B to 38% B in 12 min, 38% B; Wave Length: 254 nm; RT1 (min): 10.53; The collected fraction was lyophilized to afford (3S)-1-({3-[(2-aminoethyl)carbamoyl]-4-{[(2S,3R,4S,5S,6S)-6-carboxy-3,4,5-trihydroxyoxan-2-yl]oxy}phenyl}methyl)-1-[(2-{6-[(2-azidoethyl)amino]-4-{3-[(4-methyl-1,2,4-triazol-3-yl)methyl]oxetan-3-yl}pyridin-2-yl}-3-oxo-7-(trifluoromethyl)-1H-isoindol-5-yl)methyl]-3-methylpiperidin-1-ium (15 mg, 14%) as a white solid. LCMS (ES, m/z): 1209.30 [M+H]⁺.

### Step 6: Synthesis of Compound 223

To a mixture of (3S)-1-({3-[(2-aminoethyl)carbamoyl]-4-{[(2S,3R,4S,5S,6S)-6-carboxy-3,4,5-trihydroxyoxan-2-yl]oxy}phenyl}methyl)-1-[(2-{6-[(2-azidoethyl)amino]-4-{3-[(4-methyl-1,2,4-triazol-3-yl)methyl]oxetan-3-yl}pyridin-2-yl}-3-oxo-7-(trifluoromethyl)-1H-isoindol-5-yl)methyl]-3-methylpiperidin-1-ium (12 mg, 0.012 mmol, 1.0 equiv) and DIEA (5 mg, 0.036 mmol, 3.0 equiv) in DMF (0.5 mL) was added bis(2,5-dioxopyrrolidin-1-yl) pentanedioate (6 mg, 0.018 mmol, 1.5 equiv) at 0° C. The resulting was stirred for 2 h at 25° C. LCMS indicated the reaction was completed. The reaction system was purified by prep-HPLC with following condition: Column: XSelect CSH Prep C18 OBD Column, 19*150 mm, 5 m; Mobile Phase A: Water (0.1% TFA), Mobile Phase B: MeOH--HPLC; Flow rate: 25 mL/min; Gradient: 33% B to 63% B in 7 min, 63% B; Wave Length: 254 nm; RT1 (min): 4.9; The collected fraction was lyophilized to afford (3S)-1-[(2-{6-[(2-azidoethyl)amino]-4-{3-[(4-methyl-1,2,4-triazol-3-yl)methyl]oxetan-3-yl}pyridin-2-yl}-3-oxo-7-(trifluoromethyl)-1H-isoindol-5-yl)methyl]-1-[(4-{[(2S,3R,4S,5S,6S)-6-carboxy-3,4,5-trihydroxyoxan-2-yl]oxy}-3-[(2-{5-[(2,5-dioxopyrrolidin-1-yl)oxy]-5-oxopentanamido}ethyl)carbamoyl]phenyl)methyl]-3-methylpiperidin-1-ium; trifluoroacetic acid (1.4 mg, 7.93%) as a white solid. LCMS (ESI, m/z): 1204.40 [M+H−TFA]⁺. ¹H NMR (400 MHz, Methanol-d4) δ 8.31 (s, 1H), 8.18 (s, 1H), 8.00 (s, 1H), 7.60-7.56 (m, 1H), 7.43 (d, J=8.6 Hz, 1H), 7.05 (d, J=1.2 Hz, 1H), 6.21 (s, 1H), 5.61 (s, 2H), 5.27 (d, J=8.0 Hz, 2H), 5.10 (d, J=8.0 Hz, 1H), 4.99-4.91 (m, 5H), 4.61 (s, 2H), 4.04 (d, J=8.0 Hz, 1H), 3.81 (s, 2H), 3.66-3.58 (m, 3H), 3.57-3.42 (m, 10H), 3.42-3.38 (m, 2JH), 2.91 (s, 3H), 2.84-2.78 (m, 4H), 2.61 (t, J=8.0 Hz, 2H), 2.31 (t, J=8.0 Hz, 2H), 2.01-1.80 (m, 6H), 1.32-1.20 (m, 1H), 1.02-1.00 (m, 4H).

### Synthesis of Compound 224

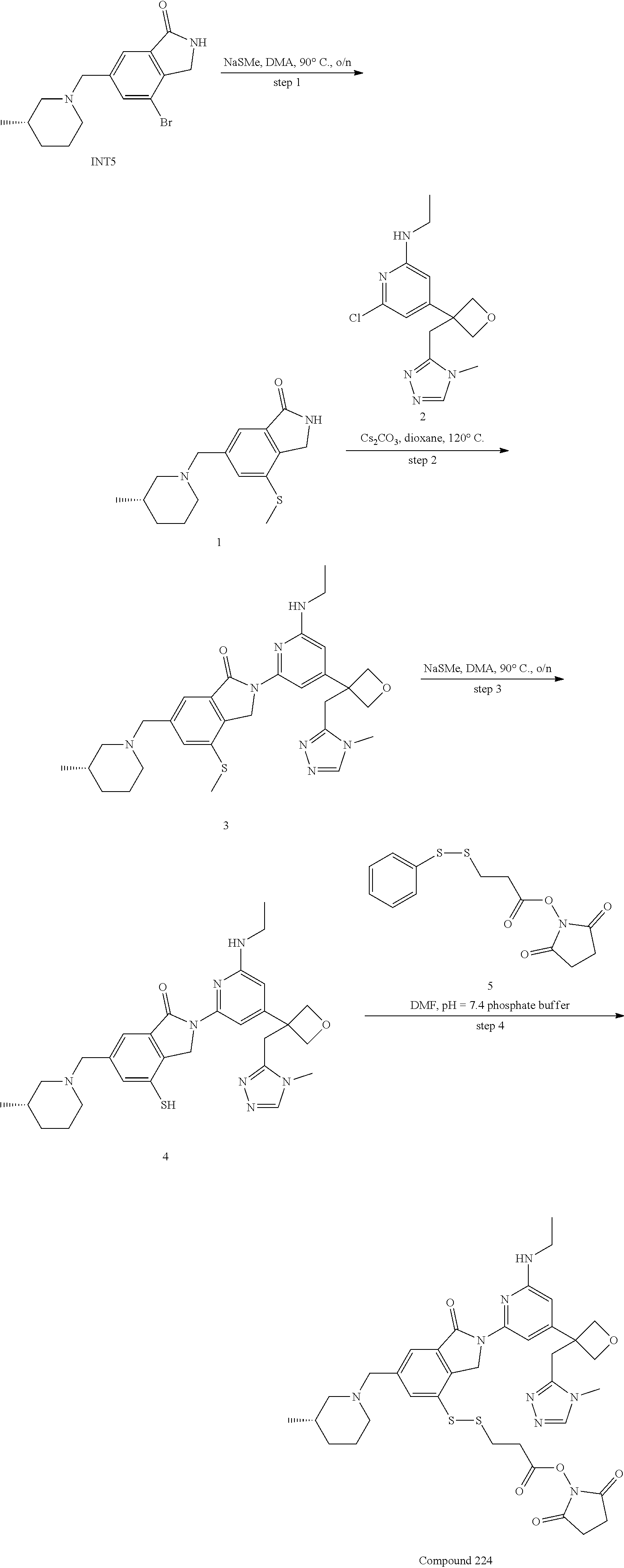

### Step 1. Synthesis of Compound 1

To a stirred solution of 4-bromo-6-{[(3S)-3-methylpiperidin-1-yl]methyl}-2,3-dihydroisoindol-1-one (300 mg, 0.92 mmol, 1 equiv) in DMA (3 mL) was added sodium thiomethoxide (97 mg, 1.39 mmol, 1.5 equiv) at room temperature under nitrogen atmosphere. The resulting mixture was stirred for 1 h at 90° C. under nitrogen atmosphere. LCMS indicated the reaction was completed. The mixture was allowed to cool down to room temperature. The residue was purified by reverse flash chromatography with the following conditions: column, C18 silica gel; mobile phase, MeCN in Water (0.05% TFA), 5% to 50% gradient in 30 min; detector, UV 254 nm. The collected fraction was concentrated to afford 6-{[(3S)-3-methylpiperidin-1-yl]methyl}-4-(methylsulfanyl)-2,3-dihydroisoindol-1-one (210 mg, 77%) as an off-white solid. LCMS (ESI, m/z):291 [M+H]⁺

### Step 2: Synthesis of Compound 3

To a stirred solution of 6-{[(3S)-3-methylpiperidin-1-yl]methyl}-4-(methylsulfanyl)-2,3-dihydroisoindol-1-one (210 mg, 0.72 mmol, 1 equiv) and 6-chloro-N-ethyl-4-{3-[(4-methyl-1,2,4-triazol-3-yl)methyl]oxetan-3-yl}pyridin-2-amine (222 mg, 0.72 mmol, 1 equiv) in dioxane (6 mL) was added Cs₂CO₃ (471 mg, 1.44 mmol, 2 equiv) Xantphos (83 mg, 0.145 mmol, 0.2 equiv) and Pd(OAC)₂ (16.23 mg, 0.072 mmol, 0.1 equiv) at room temperature under nitrogen atmosphere. The resulting mixture was stirred for 2 h at 120° C. under nitrogen atmosphere. LCMS indicated the reaction was completed. The mixture was allowed to cool down to room temperature. The residue was purified by reverse flash chromatography with the following conditions: column, C18 silica gel; mobile phase, MeCN in Water (0.1% TFA), 5% to 60% gradient in 10 min; detector, UV 254 nm. This resulted in 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (110 mg, 27%) as a yellow solid. LCMS (ESI, m/z):562[M+H]⁺

### Step 3. Synthesis of Compound 4

To a stirred solution of 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (110 mg, 0.19 mmol, 1 equiv) in DMA (2 mL) was added Sodium thiomethoxide (68 mg, 0.98 mmol, 5 equiv) in portions at room temperature under nitrogen atmosphere. The resulting mixture was stirred for 3 h at 90° C. under nitrogen atmosphere. LCMS indicated the reaction was completed. The mixture was allowed to cool down to room temperature. The residue was purified by reverse flash chromatography with the following conditions: column, C18 silica gel; mobile phase, MeCN in Water (0.05% TFA), 5% to 60% gradient in 30 min; detector, UV 254 nm. This resulted in 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-sulfanyl-3H-isoindol-1-one (55 mg, 49%) as a yellow solid. LCMS (ESI, m/z):548[M+H]⁺

### Step 4. Synthesis of Compound 224

To a stirred solution of 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-sulfanyl-3H-isoindol-1-one (50 mg, 0.091 mmol, 1 equiv) and 2,5-dioxopyrrolidin-1-yl 3-(pyridin-2-yldisulfanyl)propanoate) (34 mg, 0.11 mmol, 1.2 equiv) in DMF (500 uL) was added dropwise at room temperature under nitrogen atmosphere. LCMS indicated the reaction was completed. The resulting mixture was stirred for 2 h at room temperature under nitrogen atmosphere. The reaction mixture was purified by Prep-HPLC with the following conditions (Column: XSelect CSH Prep C18 OBD Column, 19*150 mm, 5 m; Mobile Phase A: Water (0.1% TFA), Mobile Phase B: ACN; Flow rate: 25 mL/min; Gradient: 15% B to 37% B in 10 min, 37% B; Wave Length: 254 nm; RT1 (min): 7.88; The collected fraction was lyophilized to afford 2,5-dioxopyrrolidin-1-yl 3-({2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-1-oxo-3H-isoindol-4-yl}disulfanyl)propanoate (2.3 mg, 3.36%) as a white solid. LCMS (ESI, m/z):749[M+H−TFA]^{+ 1}H NMR (400 MHz, CD₃OD) δ 8.80-8.60 (m, 1H), 8.13 (s, 1H), 7.90 (s, 1H), 7.20 (s, 1H), 5.96 (br s, 1H), 5.17-5.15 (m, 2H), 5.02 (s, 4H), 4.49 (s, 2H), 3.74 (s, 2H), 3.52-3.36 (m, 4H), 3.23-3.11 (m, 4H), 2.95-2.86 (m, 1H), 2.82-2.80 (m, 4H), 2.79-2.74 (m, 2H), 1.97-1.74 (m, 4H), 1.28-1.15 (m, 4H), 1.02-1.00 (m, 3H).

### Synthesis of Compound 225

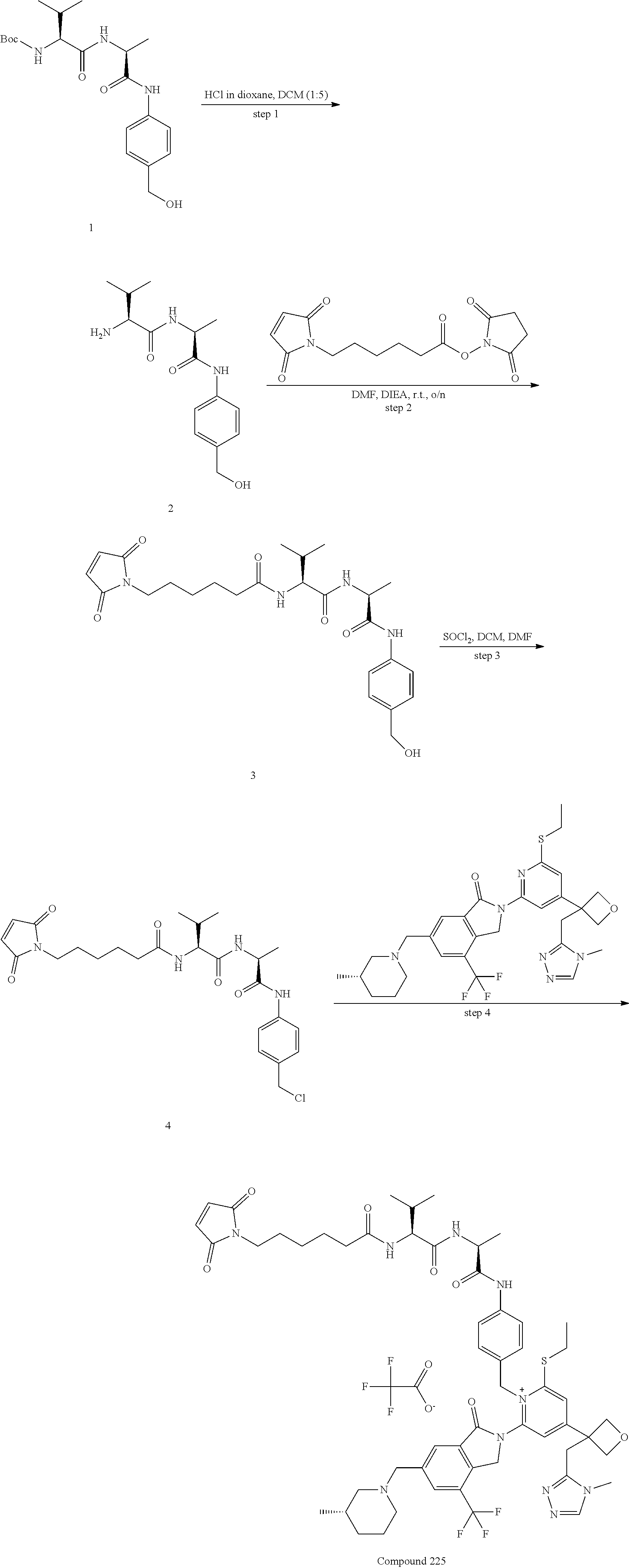

### Step 1: Synthesis of Compound 2

To a stirred solution of tert-butyl N-[(1S)-1-{[(1S)-1-{[4-(hydroxymethyl)phenyl]carbamoyl}ethyl]carbamoyl}-2-methylpropyl]carbamate (400 mg, 1.01 mmol, 1 equiv) in DCM (5 mL) was added HCl (g, 4N in dioxane, 1 mL) dropwise at 0° C. under air atmosphere. The resulting mixture was stirred for 2 h at room temperature under air atmosphere. LCMS indicated the reaction was completed. The resulting mixture was concentrated under reduced pressure. The residue was re-dissolved in DCM (5 mL) and concentrated under reduced pressure. The crude product mixture was used in the next step directly without further purification. LCMS:(ES·m/z):294[M+1]⁺.

### Step 2: Synthesis of Compound 3

To a stirred mixture of (2S)-2-amino-N-[(1S)-1-{[4-(hydroxymethyl)phenyl]carbamoyl}ethyl]-3-methylbutanamide (400 mg, 1.36 mmol, 1 equiv) and 2,5-dioxopyrrolidin-1-yl 6-(2,5-dioxopyrrol-1-yl)hexanoate (504 mg, 1.63 mmol, 1.2 equiv) in DMF (5 mL) was added DIEA (352 mg, 2.72 mmol, 2 equiv) dropwise at 0° C. The resulting mixture was stirred for 12 h at room temperature under air atmosphere. LCMS indicated the reaction was completed. The reaction mixture was purified by reverse flash chromatography with the following conditions: column, silica gel; mobile phase, ACN in water (0.05% TFA), 5% to 50% gradient in 30 min; detector, UV 254 nm. The collected fraction was concentrated to afford 6-(2,5-dioxopyrrol-1-yl)-N-[(1S)-1-{[(1S)-1-{[4-(hydroxymethyl)phenyl]carbamoyl}ethyl]carbamoyl}-2-methylpropyl]hexanamide (165 mg, 24%) as a yellow solid. LCMS:(ES·m/z):487[M+1]⁺.

### Step 3: Synthesis of Compound 4

To a stirred solution of 6-(2,5-dioxopyrrol-1-yl)-N-[(1S)-1-{[(1S)-1-{[4-(hydroxymethyl)phenyl]carbamoyl}ethyl]carbamoyl}-2-methylpropyl]hexanamide (1.9 g, 3.90 mmol, 1 equiv) in DCM (19 mL) was added SOCl₂ (0.93 g, 7.81 mmol, 2 equiv) in portions at 0° C. under nitrogen atmosphere. The resulting mixture was stirred for 2 h at 0° C. under air atmosphere. LCMS indicated the reaction was completed. The resulting mixture was concentrated under reduced pressure. The resulting mixture was concentrated under reduced pressure. The residue was purified by silica gel column chromatography, eluted with CH₂Cl₂/MeOH (10:1) to afford N-[(1S)-1-{[(1S)-1-{[4-(chloromethyl)phenyl]carbamoyl}ethyl]carbamoyl}-2-methylpropyl]-6-(2,5-dioxopyrrol-1-yl)hexanamide (1 g, 48%) as a white solid. LCMS:(ES·m/z):505,507[M+1]⁺.

### Step 4: Synthesis of Compound 225

To a stirred mixture of N-[(1S)-1-{[(1S)-1-{[4-(chloromethyl)phenyl]carbamoyl}ethyl]carbamoyl}-2-methylpropyl]-6-(2,5-dioxopyrrol-1-yl)hexanamide (200 mg, 0.39 mmol, 1 equiv) and 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-4-(trifluoromethyl)-3H-isoindol-1-one (200 mg, 0.33 mmol, 0.84 equiv) in acetone (8 mL) was added KI (120 mg, 0.72 mmol, 1.83 equiv) at room temperature. The resulting mixture was stirred for 3 h at 50° C. LCMS indicated the reaction 20% product. The reaction mixture was purified by reverse flash chromatography with the following conditions: column, C18 silica gel; mobile phase, MeCN in Water (0.1% TFA), 3% to 60% gradient in 30 min; detector, UV 254 nm. The crude product (100 mg) was purified by Prep-HPLC with the following conditions (Column: XBridge Prep C18 OBD Column, 19*150 mm, 5 m; Mobile Phase A: Water (0.05% TFA), Mobile Phase B: ACN; Flow rate: 25 mL/min; Gradient: 30% B to 51% B in 13 min, 51% B to 51% B in 14 min, 51% B; Wave Length: 254 nm; RT1 (min): 7.8/9.38/1). The collected fraction was lyophilized to afford (3S)-1-({4-[(2S)-2-[(2S)-2-[6-(2,5-dioxopyrrol-1-yl)hexanamido]-3-methylbutanamido]propanamido]phenyl}methyl)-1-({2-[6-(ethylsulfanyl)-4-{3-[(4-methyl-1,2,4-triazol-3-yl)methyl]oxetan-3-yl}pyridin-2-yl]-3-oxo-7-(trifluoromethyl)-1H-isoindol-5-yl}methyl)-3-methylpiperidin-1-ium trifluoroacetate (47 mg, 11%) as a white solid. LCMS:(ES·m/z):1069[M+H−TFA]⁺. ¹H NMR (400 MHz, DMSO-d₆) δ10.05-9.91 (m, 3H), 8.29-8.23 (m, 3H), 8.17 (d, J=6.8 Hz, 1H), 7.84 (d, J=8.4 Hz, 1H), 7.59 (d, J=8.4 Hz, 2H), 7.32 (d, J=8.4 Hz, 2H), 7.09-7.00 (m, 3H), 5.45 (s, 2H), 5.33 (s, 2H), 4.88-4.82 (m, 4H), 4.54 (s, 2H), 4.33 (t, J=6.8 Hz, 1H), 4.15 (t, J=6.8 Hz, 1H), 3.75-3.70 (m, 4H), 3.37-3.34 (m, 4H), 3.19-3.14 (m, 2H), 2.90-2.80 (m, 1H), 2.65-2.55 (m, 1H), 2.20-2.10 (m, 2H), 1.98-62 (m, 5H), 1.55-1.45 (m, 4H), 1.42-1.25 (m, 7H), 1.20-1.08 (m, 2H), 1.05-1.01 (m, 1H), 0.88-0.81 (m, 9H).

### Synthesis of Compound 226

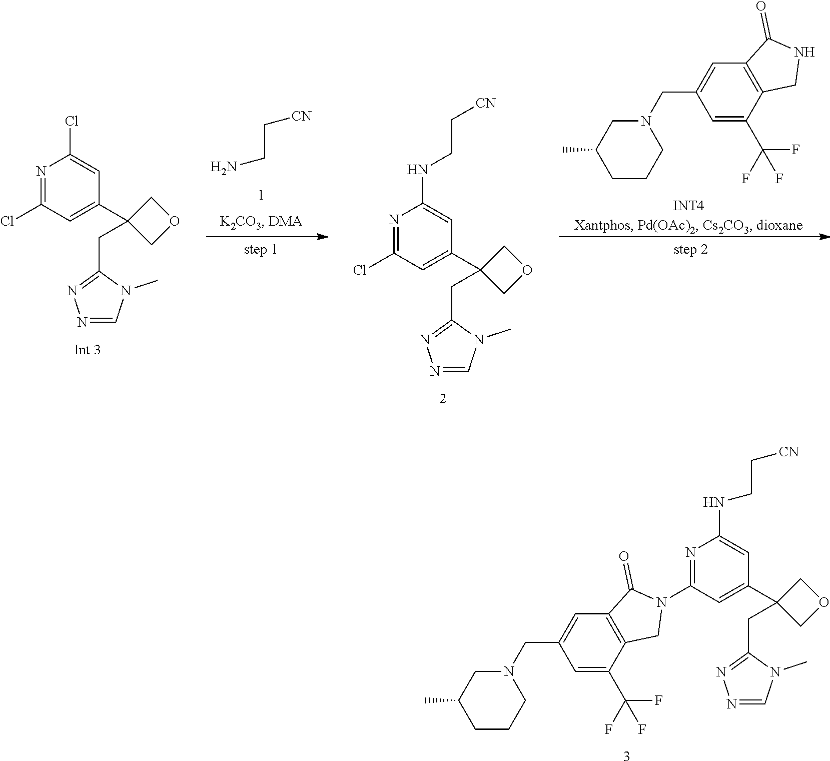

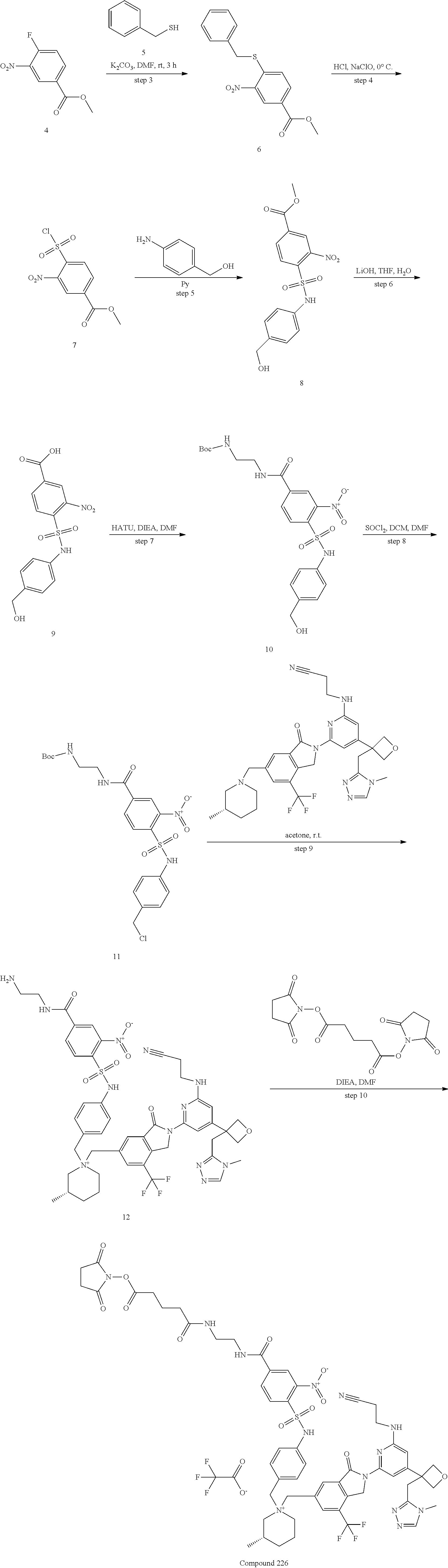

### Step 1: Synthesis of Compound 2

To a stirred solution of 2,6-dichloro-4-{3-[(4-methyl-1,2,4-triazol-3-yl)methyl]oxetan-3-yl}pyridine (1.0 g, 3.34 mmol, 1.0 equiv) and β-aminopropionitrile (4.69 g, 66.90 mmol, 20.0 equiv) in DMA (4.0 mL) was added K₂CO₃ (0.92 g, 6.68 mmol, 2.0 equiv) in portion at room temperature under nitrogen atmosphere. The resulting mixture was stirred for two days at 120° C. under nitrogen atmosphere. ˜70% desired product could be by LCMS. The reaction was cooled to room temperature and quenched with water at room temperature. The aqueous layer was extracted with CH₂Cl₂ (3×100 mL). The resulting mixture was concentrated under vacuum. The residue was purified by silica gel column chromatography, eluted with CH₂Cl₂/MeOH (5:1) to afford 3-[(6-chloro-4-{3-[(4-methyl-1,2,4-triazol-3-yl)methyl]oxetan-3-yl}pyridin-2-yl)amino]propanenitrile (600 mg, 49%) as a light green solid. LCMS: (ES, m/s): 333,335 [M+H]⁺.

### Step 2: Synthesis of Compound 3

To a stirred solution of 6-{[(3S)-3-methylpiperidin-1-yl]methyl}-4-(trifluoromethyl)-2,3-dihydroisoindol-1-one (187 mg, 0.60 mmol, 1.0 equiv) and 3-[(6-chloro-4-{3-[(4-methyl-1,2,4-triazol-3-yl)methyl]oxetan-3-yl}pyridin-2-yl)amino]propanenitrile (200 mg, 0.60 mmol, 1.0 equiv) in dioxane (8.0 mL) was added Xantphos (139 mg, 0.24 mmol, 0.4 equiv), Pd(OAc)₂ (54 mg, 0.24 mmol, 0.4 equiv) and Cs₂CO₃ (587 mg, 1.80 mmol, 3.0 equiv) in portions at room temperature under nitrogen atmosphere. The resulting mixture was stirred for 1 h at 120° C. under nitrogen atmosphere. LCMS indicated the reaction was completed. The resulting mixture was filtered, the filter cake was washed with ACN and DCM. The filtrate was concentrated under reduced pressure. The residue was purified by silica gel column chromatography, eluted with CH₂Cl₂/MeOH (5:1) to afford 3-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]propanenitrile; formic acid (220 mg, 53%) as a green solid. LCMS:(ES, m/s):609[M+H]⁺, 631 [M+Na]⁺.

### Step 3: Synthesis of Compound 6

To a stirred mixture of methyl 4-fluoro-3-nitrobenzoate (10 g, 50.21 mmol, 1 equiv) and K₂CO₃ (13.88 g, 100.43 mmol, 2 equiv) in DMF (160 mL) was added benzyl mercaptan (12.47 g, 100.43 mmol, 2 equiv) dropwise at 0° C. The resulting mixture was stirred for 3 h at 25° C. LCMS indicated the reaction was completed. The reaction was quenched by the addition of Water (450 mL) at 0° C. The precipitated solids were collected by filtration and washed with water (3×150 mL). The solid was purified by trituration with PE (300 mL). This resulted in methyl 4-(benzylsulfanyl)-3-nitrobenzoate (10 g, 65%) as a yellow solid. LCMS (ES, m/z): 304 [M+H]⁺

### Step 4: Synthesis of Compound 7

To a stirred mixture of methyl 4-(benzylsulfanyl)-3-nitrobenzoate (10 g, 36.32 mmol, 1 equiv) in DCM (200 mL) was added HCl (6N, 200 mL) and NaClO (100 mL) dropwise at 0° C. The resulting mixture was stirred for 1 h at 0° C. LCMS indicated the reaction was completed. The resulting mixture was extracted with CH₂Cl₂ (3×20 mL). The combined organic layers were washed with brine (300 mL), dried over anhydrous Na₂SO₄. After filtration, the filtrate was concentrated under reduced pressure. The residue was purified by silica gel column chromatography, eluted with PE/EA (1:4) to afford methyl 4-(chlorosulfonyl)-3-nitrobenzoate (8 g, 78%) as a yellow solid. ¹H NMR (300 MHz, CDCl₃) δ 8.51-8.46 (m, 2H), 8.38 (d, J=8.1 Hz, 1H), 4.06 (s, 3H)

### Step 5: Synthesis of Compound 8

To a stirred solution of methyl 4-(chlorosulfonyl)-3-nitrobenzoate (1.2 g, 4.29 mmol, 1 equiv) in Pyridine (12 mL) was added β-aminobenzylalcohol (0.63 g, 5.14 mmol, 1.2 equiv) in portions at 0° C. under air atmosphere. The resulting mixture was stirred for 30 min at 0° C. under air atmosphere. 50% desired product was found by LCMS. The mixture was acidified to pH 3 with 1N HCl. The precipitated solids were collected by filtration and washed with Et₂O (3×50 mL). The solid was purified by silica gel column chromatography, eluted with CH₂Cl₂/MeOH (10:1) to afford methyl 4-{[4-(hydroxymethyl)phenyl]sulfamoyl}-3-nitrobenzoate (850 mg, 49%) as a yellow solid. LCMS:(ES·m/z):349[M+H−H₂O]⁺.

### Step 6: Synthesis of Compound 9

To a stirred solution of methyl 4-{[4-(hydroxymethyl)phenyl]sulfamoyl}-3-nitrobenzoate (850 mg, 2.32 mmol, 1 equiv) in THE (8 mL) was added LiGH (222 mg, 9.28 mmol, 4 equiv) in H₂O (4 mL) dropwise at 0° C. The resulting mixture was stirred for 2 h at 0° C. under air atmosphere. LCMS indicated the reaction was completed. The reaction mixture was acidified to pH 3 with 1N HCl. The precipitated solids were collected by filtration and washed with Et₂O (3×50 mL). This resulted in 4-{[4-(hydroxymethyl)phenyl]sulfamoyl}-3-nitrobenzoic acid (270 mg, 29%) as a yellow solid. LCMS:(ES·m/z):335[M+H−H₂O]⁺.

### Step 7: Synthesis of Compound 10

To a stirred mixture of 4-{[4-(hydroxymethyl)phenyl]sulfamoyl}-3-nitrobenzoic acid (1 g, 2.83 mmol, 1 equiv) and HATU (1.40 g, 3.68 mmol, 1.30 equiv) in DMF (10 mL) was stirred for 15 min at 0° C. under air atmosphere. To the above mixture was added 1-(2-aminoethyl)pyrrole-2,5-dione (300 mg, 2.14 mmol, 1.00 equiv) in portions and DIEA (1.10 g, 8.51 mmol, 3.00 equiv) dropwise at 0° C. The resulting mixture was stirred for additional 2 h at 0° C. LCMS indicated the reaction was 30% product. The reaction was quenched with Water at 0° C. The resulting mixture was extracted with EtOAc (3×80 mL). The combined organic layers were washed with brine (2×30 mL), dried over anhydrous Na₂SO₄. After filtration, the filtrate was concentrated under reduced pressure. The residue was purified by silica gel column chromatography, eluted with CH₂Cl₂/MeOH (10:1) to afford tert-butyl N-{2-[(4-{[4-(hydroxymethyl)phenyl]sulfamoyl}-3-nitrophenyl)formamido]ethyl}carbamate (0.8 g, 510%) as a yellow solid. LCMS:(ES·m/z):477[M+H−H₂O]⁺.

### Step 8: Synthesis of Compound 11

The stirred solution of tert-butyl N-{2-[(4-{[4-(hydroxymethyl)phenyl]sulfamoyl}-3-nitrophenyl)formamido]ethyl}carbamate (50 mg, 0.10 mmol, 1 equiv) in DCM (2.5 mL) was added SOCl₂ (25 mg, 0.21 mmol, 2.08 equiv) and 1 drop DMF at 0° C. under nitrogen atmosphere. The resulting mixture was stirred for 2 h at 0° C. LCMS indicated the reaction was completed. The resulting mixture was concentrated under reduced pressure. The crude product was used in the next step directly without further purification. The reaction was run twice in parallel. LCMS:(ms, ESI): 477[M+H−HCl]⁺

### Step 9: Synthesis of Compound 12

The mixture of tert-butyl N-{2-[(4-{[4-(chloromethyl)phenyl]sulfamoyl}-3-nitrophenyl)formamido]ethyl}carbamate (50 mg, 0.097 mmol, 1 equiv) and 3-[(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)amino]propanenitrile (55 mg, 0.090 mmol, 0.93 equiv) in acetone (2 mL) was stirred for 1 h at 0° C. LCMS indicated ˜20% desired product. The reaction was run twice in parallel. The reaction mixture was purified by reverse flash chromatography with the following conditions: column, C18 silica gel; mobile phase, MeCN in Water (0.1% TFA), 5% to 50% gradient in 40 min; detector, UV 254 nm. This resulted in (3S)-1-[(4-{4-[(2-aminoethyl)carbamoyl]-2-nitrobenzenesulfonamido}phenyl)methyl]-1-[(2-{6-[(2-cyanoethyl)amino]-4-{3-[(4-methyl-1,2,4-triazol-3-yl)methyl]oxetan-3-yl}pyridin-2-yl}-3-oxo-7-(trifluoromethyl)-1H-isoindol-5-yl)methyl]-3-methylpiperidin-1-ium (20 mg, 9%) as an off-white solid. LCMS(ESI, ms):985[M]⁺

### Step 10: Synthesis of Compound 226

To the stirred mixture of (3S)-1-[(4-{4-[(2-aminoethyl)carbamoyl]-2-nitrobenzenesulfonamido}phenyl)methyl]-1-[(2-{6-[(2-cyanoethyl)amino]-4-{3-[(4-methyl-1,2,4-triazol-3-yl)methyl]oxetan-3-yl}pyridin-2-yl}-3-oxo-7-(trifluoromethyl)-1H-isoindol-5-yl)methyl]-3-methylpiperidin-1-ium (20 mg, 0.020 mmol, 1 equiv) and bis(2,5-dioxopyrrolidin-1-yl) pentanedioate (8.01 mg, 0.024 mmol, 1.21 equiv) in DMF (1 mL) was added DIEA (8.00 mg, 0.061 mmol, 3.05 equiv) at 0° C. The resulting mixture was stirred for 2 h at 0° C. The reaction mixture was purified by Prep-HPLC with the following conditions (Column: XSelect CSH Prep C18 OBD Column, 19*150 mm, 5 m; Mobile Phase A: Water (0.05% TFA), Mobile Phase B: ACN; Flow rate: 25 mL/min; Gradient: 20% B to 37% B in 10 min, 37% B; Wave Length: 254 nm; RT1 (min): 9.6; The collected fraction was lyophilized to afford (3S)-1-[(2-{6-[(2-cyanoethyl)amino]-4-{3-[(4-methyl-1,2,4-triazol-3-yl)methyl]oxetan-3-yl}pyridin-2-yl}-3-oxo-7-(trifluoromethyl)-1H-isoindol-5-yl)methyl]-1-[(4-{4-[(2-{5-[(2,5-dioxopyrrolidin-1-yl)oxy]-5-oxopentanamido}ethyl)carbamoyl]-2-nitrobenzenesulfonamido}phenyl)methyl]-3-methylpiperidin-1-ium trifluoroacetate (5.8 mg, 19%) as a white solid. LCMS:(ESI, ms):1196[M−TFA]^{+ 1}H NMR (300 MHz, DMSO-d₆) δ 10.81 (s, 1H), 10.03 (br s, 1H), 8.88 (s, 1H), 8.25-8.12 (m, 6H), 8.06-8.03 (m, 2H), 7.46 (s, 1H), 7.14-7.06 (m, 4H), 6.16 (s, 1H), 5.15 (s, 2H), 4.87 (d, J=6.3 Hz, 2H), 4.73 (d, J=6.3 Hz, 2H), 4.63 (s, 2H), 4.51 (s, 2H), 3.84 (s, 2H), 3.35-3.20 (m, 9H), 3.08 (s, 3H), 2.84-2.80 (m, 7H), 2.72-2.65 (m, 2H), 2.17-2.15 (m, 2H), 1.95-1.71 (m, 6H), 1.07-1.03 (m, 1H), 0.88 (d, J=6.3 Hz, 3H).

### Synthesis of Compound 227

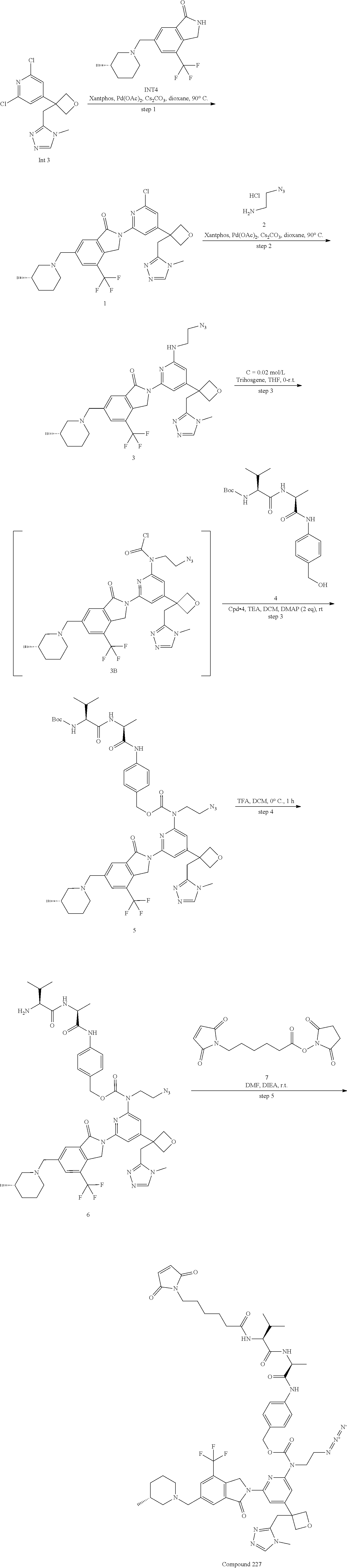

### Step 1: Synthesis of Compound 1

To a stirred mixture of 2,6-dichloro-4-{3-[(4-methyl-1,2,4-triazol-3-yl)methyl]oxetan-3-yl}pyridine (800 mg, 2.67 mmol, 1 equiv) and 6-{[(3S)-3-methylpiperidin-1-yl]methyl}-4-(trifluoromethyl)-2,3-dihydroisoindol-1-one (835 mg, 2.67 mmol, 1 equiv) in dioxane (10 mL) was added Cs₂CO₃ (1742 mg, 5.34 mmol, 2 equiv) in portions and Xantphos (309 mg, 0.53 mmol, 0.2 equiv) and Pd(OAc)₂ (60 mg, 0.26 mmol, 0.1 equiv) at room temperature under air atmosphere. The resulting mixture was stirred for 1 h at 90° C. under nitrogen atmosphere. LCMS indicated the reaction was 85% product. The mixture was allowed to cool down to room temperature. The resulting mixture was filtered, the filter cake was washed with EtOAc (3×50 mL). The filtrate was concentrated under reduced pressure. The residue was purified by silica gel column chromatography, eluted with CH₂Cl₂/MeOH (10:1) to afford 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(trifluoromethyl)-3H-isoindol-1-one (500 mg, 32%) as a yellow solid. LCMS:(ES·m/z): 575,577[M+H]⁺.

### Step 2: Synthesis of Compound 3

To a stirred mixture of 2-(6-chloro-4-{3-[(4-methyl-1,2,4-triazol-3-yl)methyl]oxetan-3-yl}pyridin-2-yl)-6-{[(3S)-3-methylpiperidin-1-yl]methyl}-4-(trifluoromethyl)-3H-isoindol-1-one (800 mg, 1.39 mmol, 1 equiv) and 2-azidoethanamine (143 mg, 1.66 mmol, 1.2 equiv) in dioxane (8 mL) was added Cs₂CO₃ (906 mg, 2.78 mmol, 2 equiv) and Xantphos (161 mg, 0.27 mmol, 0.2 equiv) and Pd(OAc)₂ (31 mg, 0.14 mmol, 0.1 equiv) in portions at room temperature under air atmosphere. The resulting mixture was stirred for 1.5 h at 90° C. under nitrogen atmosphere. LCMS indicated the reaction was 50% product. The mixture was allowed to cool down to room temperature. The resulting mixture was concentrated under reduced pressure. The residue was purified by silica gel column chromatography, eluted with CH₂Cl₂/MeOH (10:1) to afford 2-{6-[(2-azidoethyl)amino]-4-{3-[(4-methyl-1,2,4-triazol-3-yl)methyl]oxetan-3-yl}pyridin-2-yl}-6-{[(3R)-3-methylpiperidin-1-yl]methyl}-4-(trifluoromethyl)-3H-isoindol-1-one; trifluoroacetic acid (220 mg, 21%) as a yellow solid. LCMS:(ES·m/z):625[M+H]⁺.

### Step 3: Synthesis of Compound 6

To a stirred solution of 2-{6-[(2-azidoethyl)amino]-4-{3-[(4-methyl-1,2,4-triazol-3-yl)methyl]oxetan-3-yl}pyridin-2-yl}-6-{[(3S)-3-methylpiperidin-1-yl]methyl}-4-(trifluoromethyl)-3H-isoindol-1-one (260 mg, 0.41 mmol, 1 equiv) in THE (13 mL) were added Triphosgene (181 mg, 0.61 mmol, 1.47 equiv) in portions at room temperature under nitrogen atmosphere. The resulting mixture was stirred for additional 30 min at room temperature. The resulting mixture was concentrated under vacuum. The residue was re-dissolved in DCM (13 mL). To the above mixture was added tert-butyl N-[(1S)-1-{[(1S)-1-{[4-(hydroxymethyl)phenyl]carbamoyl}ethyl]carbamoyl}-2-methylpropyl]carbamate (129 mg, 0.32 mmol, 0.79 equiv) and TEA (130 mg, 1.23 mmol, 3.0 equiv) at 0° C. The resulting mixture was stirred for additional 10 min at 0° C. Finally add DMAP (104 mg, 0.85 mmol, 2.05 equiv) in portion. The resulting mixture was stirred for overnight at room temperature under nitrogen atmosphere. ˜10% desire product could be detected by LCMS. The resulting mixture was concentrated under vacuum. The residue was purified by reverse flash chromatography with the following conditions: column, C18 silica gel; mobile phase, ACN in water (0.05% TFA), 0% to 50% gradient in 30 min; detector, UV 254 nm and 220 nm. The resulting mixture was concentrated under vacuum. The residue was lyophilized to afford {4-[(2S)-2-[(2S)-2-[(tert-butoxycarbonyl)amino]-3-methylbutanamido]propanamido]phenyl}methyl N-(2-azidoethyl)-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (120 mg, 24%) as a green solid. LCMS: (ES, m/s): 523 [M/2+H]⁺, 1044 [M+H]⁺.

### Step 4: Synthesis of Compound 6

To a stirred mixture of {4-[(2S)-2-[(2S)-2-[(tert-butoxycarbonyl)amino]-3-methylbutanamido]propanamido]phenyl}methyl N-(2-azidoethyl)-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (120 mg, 0.11 mmol, 1 equiv) in DCM (6 mL) was added TFA (2 mL) dropwise at 0° C. The resulting mixture was stirred for 1 h at 0° C. under nitrogen atmosphere. LCMS indicated the reaction was completed. The resulting mixture was concentrated under vacuum. The crude product was used in the next step directly without further purification. LCMS:(ES, m/s): 944[M+H]⁺

### Step 5: Synthesis of Compound 227

To a stirred mixture of {4-[(2S)-2-[(2S)-2-amino-3-methylbutanamido]propanamido]phenyl}methyl N-(2-azidoethyl)-N-(4-{3-[(4-methyl-1,2,4-triazol-3-yl)methyl]oxetan-3-yl}-6-(6-{[(3S)-3-methylpiperidin-1-yl]methyl}-1-oxo-4-(trifluoromethyl)-3H-isoindol-2-yl)pyridin-2-yl)carbamate (110 mg, 0.11 mmol, 1 equiv) and 2,5-dioxopyrrolidin-1-yl 6-(2,5-dioxopyrrol-1-yl)hexanoate (44 mg, 0.14 mmol, 1.2 equiv) in DMF (2 mL) was added DIEA (44 mg, 0.34 mmol, 2.92 equiv) dropwise at 0° C. under nitrogen atmosphere. The resulting mixture was stirred for 3 h at room temperature under nitrogen atmosphere. LCMS indicated the reaction was completed. The reaction mixture was purified by Prep-HPLC with the following condition: Column: Column: XSelect CSH Prep C18 OBD Column, 19*250 mm, 5 m; Mobile Phase A: Water (0.05% TFA), Mobile Phase B: ACN; Flow rate: 25 mL/min; Gradient: 26% B to 41% B in 12 min. 41% B; Wave Length: 254 nm; RT1 (min): 11.33; The collected fraction was immediately lyophilized to afford trifluoroacetic acid; {4-[(2S)-2-[(2S)-2-[6-(2,5-dioxopyrrol-1-yl)hexanamido]-3-methylbutanamido]propanamido]phenyl}methyl N-(2-azidoethyl)-N-(4-{3-[(4-methyl-1,2,4-triazol-3-yl)methyl]oxetan-3-yl}-6-(6-{[(3R)-3-methylpiperidin-1-yl]methyl}-1-oxo-4-(trifluoromethyl)-3H-isoindol-2-yl)pyridin-2-yl)carbamate (30.4 mg, 19.60%) as a white solid. LCMS:(ms, ESI): 569 [M12+H]⁺, 1137[M+H]⁺H-NMR (300 MHz, DMSO-d₆): 10.02 (s, 1H), 9.76 (br s, 1H), 8.47 (br s, 1H), 8.27-8.22 (m, 3H), 8.04 (s, 1H), 7.83-7.81 (m, 1H), 7.63-7.60 (m, 2H), 7.37-7.33 (m, 3H), 6.98 (s, 2H), 5.27 (s, 2H), 5.16 (s, 2H), 4.93 (d, J=6 Hz, 2H), 4.78 (d, J=6.3 Hz, 2H), 4.53-4.50 (m, 3H), 4.39-4.37 (m, 3H), 4.16-4.13 (m, 3H), 3.61-3.56 (m, 3H), 3.35-3.33 (m, 3H), 3.28-3.27 (m, 4H), 2.87-2.84 (m, 1H), 2.59-2.56 (m, 1H), 2.16-2.08 (m, 2H), 1.98-1.60 (m, 4H), 1.51-1.47 (m, 4H), 1.31-1.29 (m, 3H), 1.25-1.04 (m, 2H), 0.87-0.81 (m, 9H).

### Synthesis of Compound 228

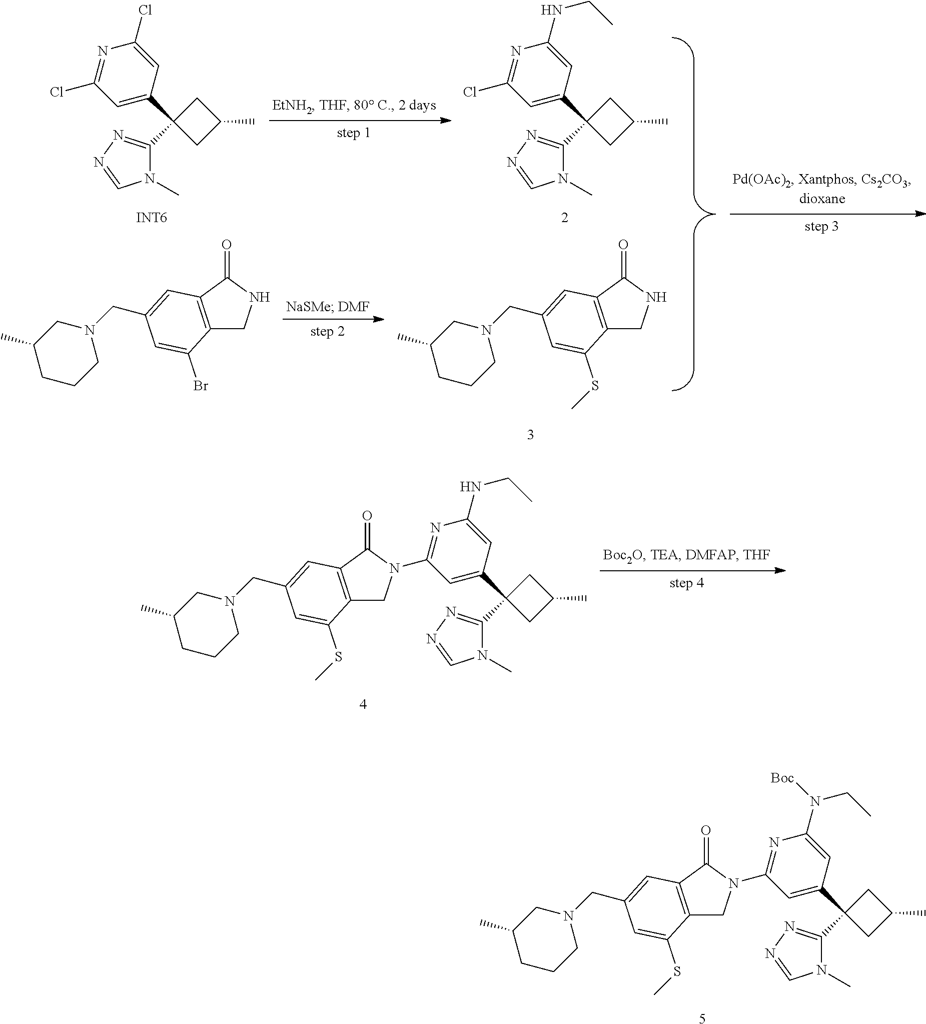

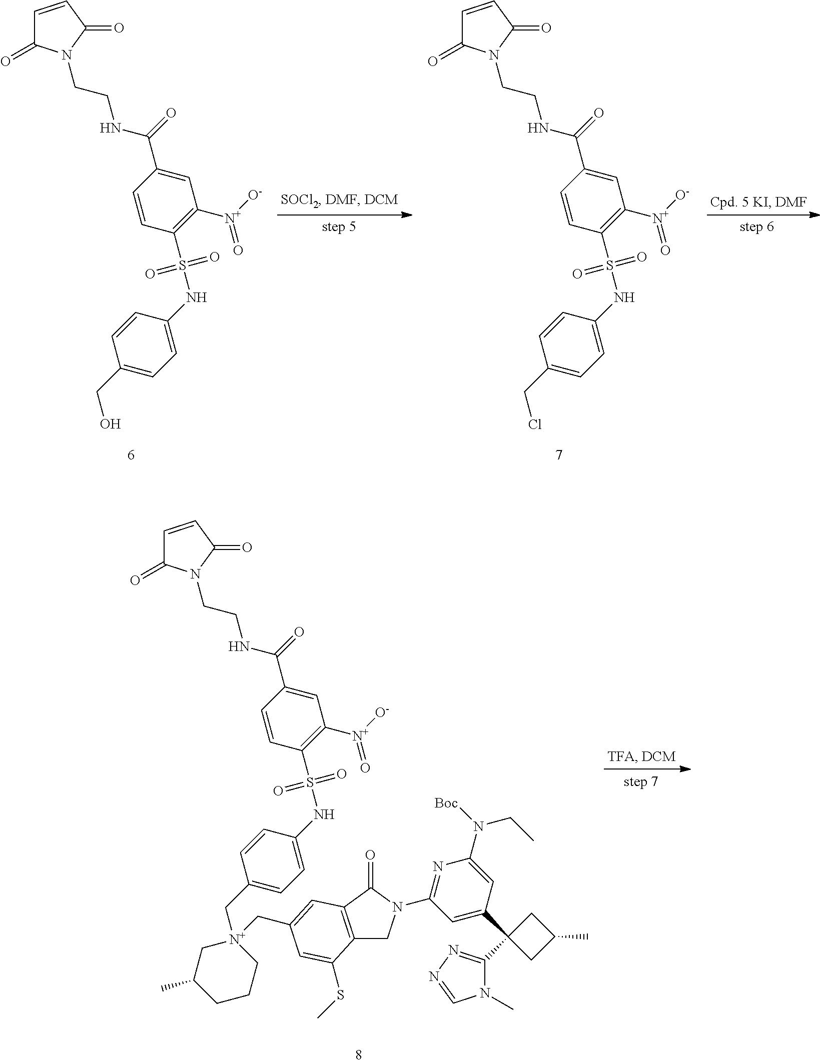 [SMILES: O=C(NCCN1C(=O)C=CC1=O)c1ccc(S(=O)(=O)Nc2ccc(CO)cc2)c([N+](=O)[O-])c1 ; O=C(NCCN1C(=O)C=CC1=O)c1ccc(S(=O)(=O)Nc2ccc(CCl)cc2)c([N+](=O)[O-])c1 ; CCN(C(=O)OC(C)(C)C)c1cc([C@]2(c3nncn3C)C[C@@H](C)C2)cc(N2Cc3c(SC)cc(C[N+]4(Cc5ccc(NS(=O)(=O)c6ccc(C(=O)NCCN7C(=O)C=CC7=O)cc6[N+](=O)[O-])cc5)CCC[C@H](C)C4)cc3C2=O)n1]

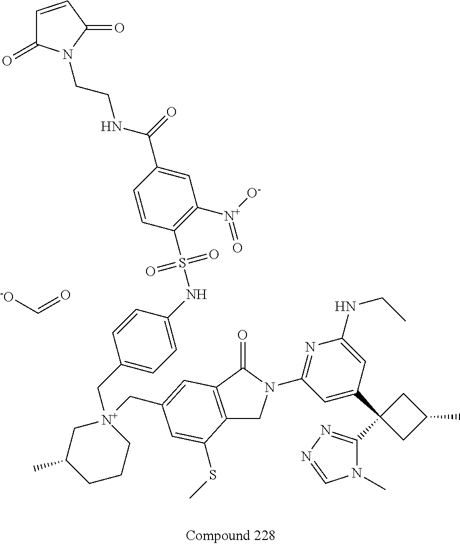

### Step 1. Synthesis of Compound 2

To a stirred solution of 2,6-dichloro-4-[3-methyl-1-(4-methyl-1,2,4-triazol-3-yl)cyclobutyl]pyridine (3 g, 10.09 mmol, 1 equiv) in Ethylamine solution (2.0 M in THF, 30 mL) was stirred for 2 days at 90° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (12:1) to afford 6-chloro-N-ethyl-4-[(1r,3s)-3-methyl-1-(4-methyl-1,2,4-triazol-3-yl)cyclobutyl]pyridin-2-amine (2.1 g, 66%) as an off-white solid.

LCMS (ESI, m/z):306,308[M+H]⁺

### Step 2. Synthesis of Compound 3

To a stirred solution of 4-bromo-6-{[(3S)-3-methylpiperidin-1-yl]methyl}-2,3-dihydroisoindol-1-one (4.5 g, 13.92 mmol, 1 equiv) in DMF (90 mL) was added MeSNa (2.93 g, 41.76 mmol, 3 equiv) in portions at room temperature. The resulting mixture was stirred for 2 h at 90° C. under nitrogen atmosphere. LCMS indicated the reaction was completed. The mixture was allowed to cool down to room temperature. The mixture was purified by reversed-phase flash chromatography with the following conditions: column, C18 silica gel; mobile phase, MeCN in Water (10 mmol/L NH₄HCO₃), 5% to 80% gradient in 30 min; detector, UV 254 nm. The collected fraction was concentrated under vacuum and lyophilized to afford 6-{[(3S)-3-methylpiperidin-1-yl]methyl}-4-(methylsulfanyl)-2,3-dihydroisoindol-1-one (3.1 g, 77%) as a white solid. LCMS (ESI, m/z):291[M+H]⁺

### Step 3: Synthesis of Compound 4

To a stirred solution of 6-chloro-N-ethyl-4-[(1r,3s)-3-methyl-1-(4-methyl-1,2,4-triazol-3-yl)cyclobutyl]pyridin-2-amine (1.7 g, 5.56 mmol, 1 equiv) 6-{[(3S)-3-methylpiperidin-1-yl]methyl}-4-(methylsulfanyl)-2,3-dihydroisoindol-1-one (1.78 g, 6.12 mmol, 1.1 equiv) and Cs₂CO₃ (3.62 g, 11.12 mmol, 2 equiv) in dioxane (34 mL) was added Xantphos (0.64 g, 1.12 mmol, 0.2 equiv) and Pd(OAc)₂ (0.12 g, 0.56 mmol, 0.1 equiv) at room temperature. The resulting mixture was stirred for 1 h at 120° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (10:1) to afford 2-[6-(ethylamino)-4-[(1r,3s)-3-methyl-1-(4-methyl-1,2,4-triazol-3-yl)cyclobutyl]pyridin-2-yl]-6-{[(3S)-3-methylpiperidin-1-yl]methyl}-4-(methylsulfanyl)-3H-isoindol-1-one (770 mg, 25%) as a white solid. LCMS (ESI, m/z):560[M+H]⁺

### Step 4: Synthesis of Compound 5

To a stirred solution of 2-[6-(ethylamino)-4-[(1r,3s)-3-methyl-1-(4-methyl-1,2,4-triazol-3-yl)cyclobutyl]pyridin-2-yl]-6-{[(3S)-3-methylpiperidin-1-yl]methyl}-4-(methylsulfanyl)-3H-isoindol-1-one (450 mg, 0.80 mmol, 1 equiv) and TEA (162 mg, 1.60 mmol, 2 equiv) in THE (9 mL) were added Boc₂O (263 mg, 1.20 mmol, 1.5 equiv) and DMAP (49 mg, 0.40 mmol, 0.5 equiv) at room temperature. The resulting mixture was stirred for overnight at room temperature under nitrogen atmosphere. LCMS indicated the reaction was completed. The resulting mixture was concentrated under vacuum. The residue was purified by silica gel column chromatography, eluted with CH₂Cl₂/MeOH (10:1) to afford tert-butyl N-ethyl-N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate (230 mg, 43%) as a brown solid. LCMS (ESI, m/z):660[M+H]⁺

### Step 5: Synthesis of Compound 7

To a stirred solution of N-[2-(2,5-dioxopyrrol-1-yl)ethyl]-4-{[4-(hydroxymethyl)phenyl]sulfamoyl}-3-nitrobenzamide (250 mg, 0.52 mmol, 1 equiv) in DCM (5 mL) was added SOCl₂ (376 mg, 3.16 mmol, 6 equiv) and DMF (0.5 mL) dropwise at 0° C. The resulting mixture was stirred for 2 h at 0° C. under nitrogen atmosphere. LCMS indicated the reaction was completed (Derivatived with MeOH). The resulting mixture was concentrated under vacuum. to afford 4-{[4-(chloromethyl)phenyl]sulfamoyl}-N-[2-(2,5-dioxopyrrol-1-yl)ethyl]-3-nitrobenzamide (250 mg, 96%) as an off-white solid. LCMS (ESI, m/z):488[M+H]⁺

### Step 6: Synthesis of Compound 8

To a stirred solution of tert-butyl N-ethyl-N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate (100 mg, 0.15 mmol, 1.00 equiv) and 4-{[4-(chloromethyl)phenyl]sulfamoyl}-N-[2-(2,5-dioxopyrrol-1-yl)ethyl]-3-nitrobenzamide (90 mg, 0.18 mmol, 1.20 equiv) in DMF (10 mL) was added KI (100 mg, 0.60 mmol, 4 equiv) at room temperature. The resulting mixture was stirred for 2 days at room temperature under nitrogen atmosphere. LCMS indicated the reaction was completed. The reaction mixture was purified by reversed-phase flash chromatography with the following conditions: column, C18 silica gel; mobile phase, MeCN in Water (0.1% TFA), 5% to 50% gradient in 30 min; detector, UV 254 nm. The collected fraction was lyophilized to afford (3S)-1-[(2-{6-[(tert-butoxycarbonyl)(ethyl)amino]-4-[(1r,3s)-3-methyl-1-(4-methyl-1,2,4-triazol-3-yl)cyclobutyl]pyridin-2-yl}-7-(methylsulfanyl)-3-oxo-1H-isoindol-5-yl)methyl]-1-{[4-(4-{[2-(2,5-dioxopyrrol-1-yl)ethyl]carbamoyl}-2-nitrobenzenesulfonamido)phenyl]methyl}-3-methylpiperidin-1-ium (160 mg, 94%) as an off-white solid. LCMS (ESI, m/z):1116[M]⁺

### Step 7: Synthesis of Compound 228

To a stirred solution of (3S)-1-[(2-{6-[(tert-butoxycarbonyl)(ethyl)amino]-4-[(1r,3s)-3-methyl-1-(4-methyl-1,2,4-triazol-3-yl)cyclobutyl]pyridin-2-yl}-7-(methylsulfanyl)-3-oxo-1H-isoindol-5-yl)methyl]-1-{[4-(4-{[2-(2,5-dioxopyrrol-1-yl)ethyl]carbamoyl}-2-nitrobenzenesulfonamido)phenyl]methyl}-3-methylpiperidin-1-ium (100 mg, 0.08 mmol, 1 equiv) in DCM (2 mL) was added TFA (1 mL) dropwise at 0° C. The resulting mixture was stirred for 2 h at 0° C. under nitrogen atmosphere. LCMS indicated the reaction was completed. The resulting mixture was concentrated under vacuum. The crude product was purified by Prep-HPLC with the following conditions (Column: XSelect CSH Prep C18 OBD Column, 19*150 mm, 5 m; Mobile Phase A: Water (0.1% FA), Mobile Phase B: ACN; Flow rate: 25 mL/min; Gradient: 18% B to 46% B in 10 min, 46% B; Wave Length: 254 nm; RT1 (min): 9.35; The collected fraction was lyophilized to afford (3S)-1-{[4-(4-{[2-(2,5-dioxopyrrol-1-yl)ethyl]carbamoyl}-2-nitrobenzenesulfonamido)phenyl]methyl}-1-({2-[6-(ethylamino)-4-[(1r,3s)-3-methyl-1-(4-methyl-1,2,4-triazol-3-yl)cyclobutyl]pyridin-2-yl]-7-(methylsulfanyl)-3-oxo-1H-isoindol-5-yl}methyl)-3-methylpiperidin-1-ium formate (6.4 mg, 6.50%) as a white solid. LCMS (ESI, m/z):1016[M+H−FA]^{+ 1}H NMR (400 MHz, DMSO-d₆) δ 8.93 (t, J=6.0 Hz, 1H), 8.22 (s, 1H), 8.14 (d, J=10.4 Hz, 1H), 8.04-7.99 (m, 2H), 7.71 (s, 1H), 7.45 (d, J=4.0 Hz, 2H), 7.16 (d, J=8.4 Hz, 2H), 7.04 (d, J=8.0 Hz, 2H), 6.97 (s, 2H), 6.06 (s, 1H), 4.76 (s, 2H), 4.58 (s, 2H), 3.59-3.55 (m, 7H), 3.06 (s, 3H), 2.77-2.71 (m, 5H), 2.56 (s, 3H), 2.50-48 (m, 2H), 1.96 (t, J=11.2 Hz, 1H), 1.67-1.43-1.42 (m, 6H), 1.13-1.03 (m, 6H), 0.87-0.80 (m, 4H).

### Synthesis of Compound 229

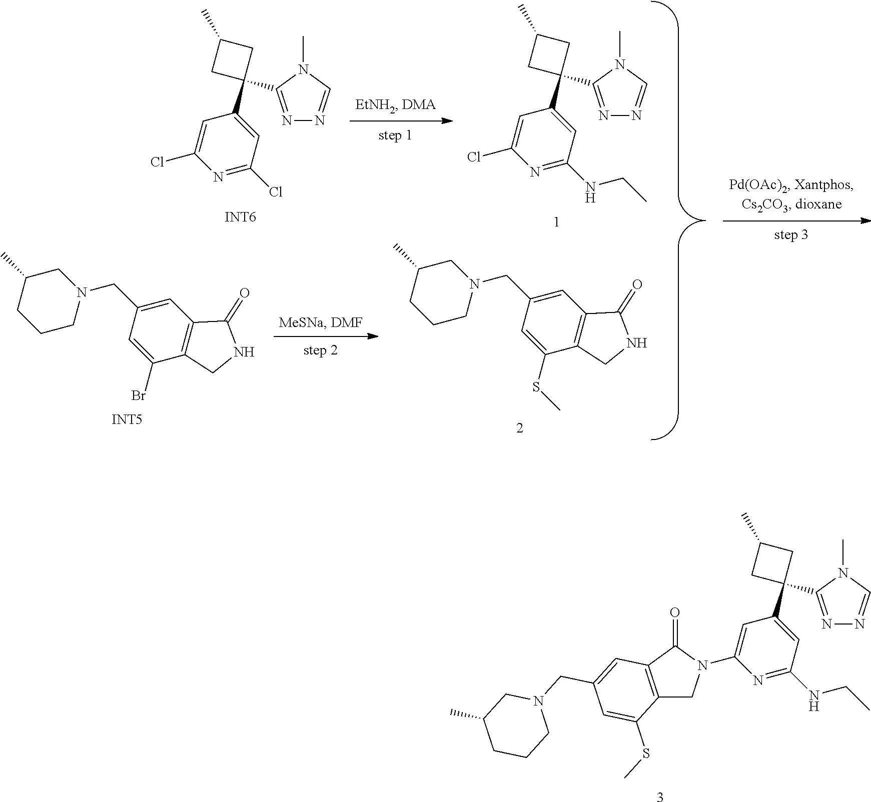

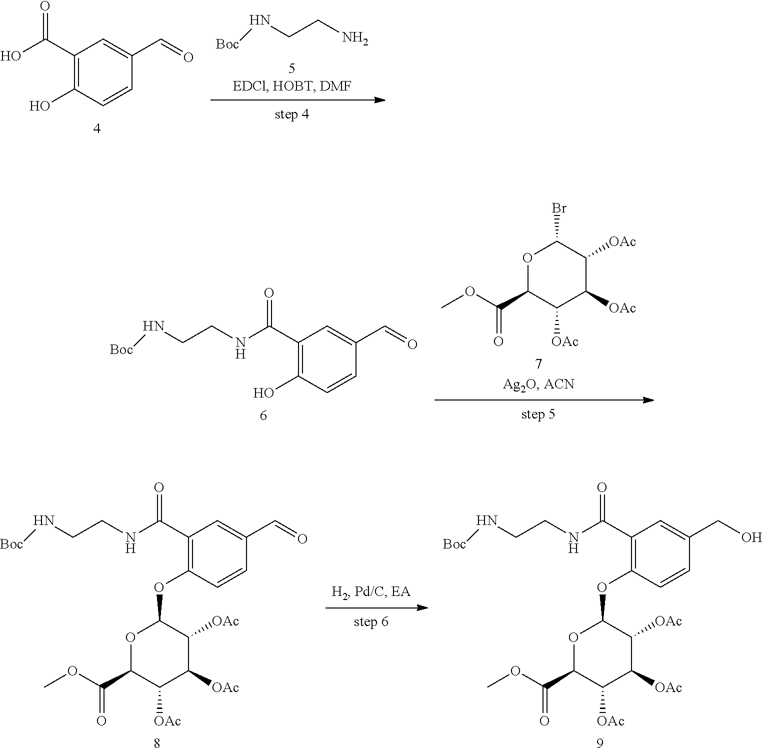

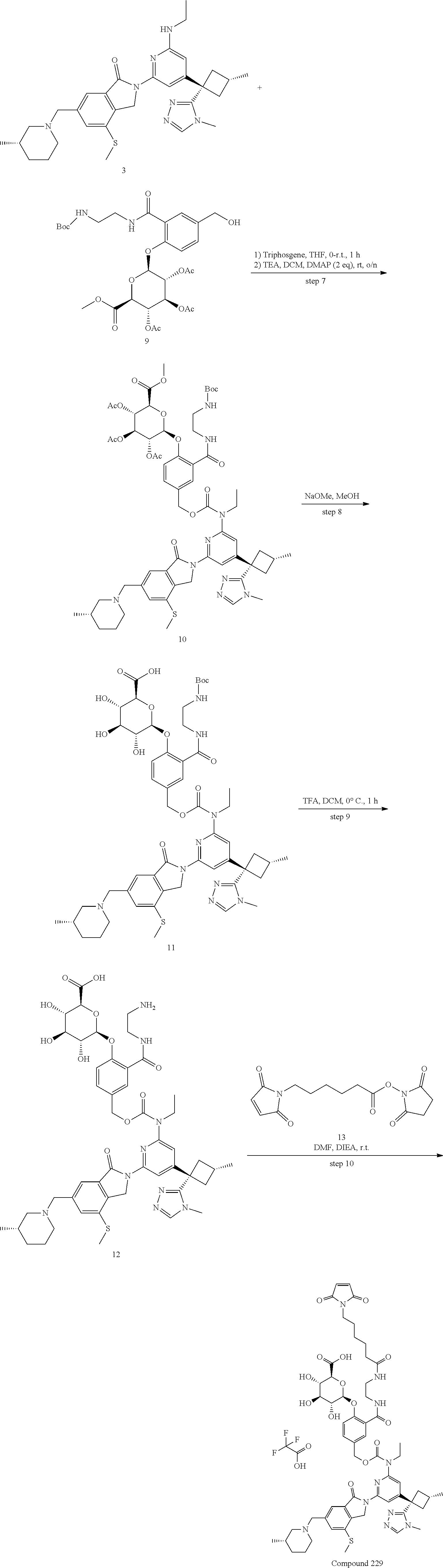

### Step 1: Synthesis of Compound 1

To a stirred solution of 2,6-dichloro-4-[3-methyl-1-(4-methyl-1,2,4-triazol-3-yl)cyclobutyl]pyridine (3 g, 10.09 mmol, 1 equiv) in Ethylamine solution (2.0 M in THF, 30 mL) was stirred for 2 days at 90° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (12:1) to afford 6-chloro-N-ethyl-4-[(1r,3s)-3-methyl-1-(4-methyl-1,2,4-triazol-3-yl)cyclobutyl]pyridin-2-amine (2.1 g, 66%) as an off-white solid. LCMS (ESI, m/z):306,308[M+H]⁺

### Step 2: Synthesis of Compound 2

To a stirred solution of 4-bromo-6-{[(3S)-3-methylpiperidin-1-yl]methyl}-2,3-dihydroisoindol-1-one (4.5 g, 13.92 mmol, 1 equiv) in DMF (90 mL) was added (methylsulfanyl)sodium (2.93 g, 41.76 mmol, 3 equiv) in portions at room temperature. The resulting mixture was stirred for 2 h at 90° C. under nitrogen atmosphere. LCMS indicated the reaction was completed. The mixture was allowed to cool down to room temperature. The mixture was purified by reversed-phase flash chromatography with the following conditions: column, C18 silica gel; mobile phase, MeCN in Water (10 mmol/L NH₄HCO₃), 5% to 80% gradient in 30 min; detector, UV 254 nm. The collected fraction was concentrated under vacuum and lyophilized to afford 6-{[(3S)-3-methylpiperidin-1-yl]methyl}-4-(methylsulfanyl)-2,3-dihydroisoindol-1-one (3.1 g, 77%) as a white solid. LCMS (ESI, m/z):291[M+H]⁺

### Step 3: Synthesis of Compound 3

To a stirred solution of 6-chloro-N-ethyl-4-[(1r,3s)-3-methyl-1-(4-methyl-1,2,4-triazol-3-yl)cyclobutyl]pyridin-2-amine (1.7 g, 5.55 mmol, 1 equiv) 6-{[(3S)-3-methylpiperidin-1-yl]methyl}-4-(methylsulfanyl)-2,3-dihydroisoindol-1-one (1.78 g, 6.11 mmol, 1.1 equiv) and Cs₂CO₃ (3.62 g, 11.11 mmol, 2 equiv) in dioxane (34 mL) was added Xantphos (0.64 g, 1.11 mmol, 0.2 equiv) and Pd(OAc)₂ (0.12 g, 0.55 mmol, 0.1 equiv) at room temperature. The resulting mixture was stirred for 1 h at 120° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (10:1) to afford 2-[6-(ethylamino)-4-[(1r,3s)-3-methyl-1-(4-methyl-1,2,4-triazol-3-yl)cyclobutyl]pyridin-2-yl]-6-{[(3S)-3-methylpiperidin-1-yl]methyl}-4-(methylsulfanyl)-3H-isoindol-1-one (770 mg, 25%) as a white solid. LCMS (ESI, m/z):560[M+H]⁺

### Step 4: Synthesis of Compound 6

A solution of 5-formyl-2-hydroxybenzoic acid (40 g, 240.77 mmol, 1 equiv), HOBT (36.04 g, 288.92 mmol, 1.2 equiv), (3-[[(ethylimino)methylidene]amino]propyl)dimethylamine hydrochloride (55.39 g, 288.9 mmol, 1.2 equiv) and tert-butyl N-(2-aminoethyl)carbamate (46.29 g, 288.92 mmol, 1.2 equiv) in DMF (400 mL) was stirred for overnight at 0° C. under nitrogen atmosphere. LCMS indicated the reaction was completed. The reaction was quenched with Water. The resulting mixture was extracted with EtOAc (3×500 mL). The combined organic layers were washed with brine (5×500 mL), dried over anhydrous Na₂SO₄. After filtration, the filtrate was concentrated under reduced pressure. The residue was purified by silica gel column chromatography, eluted with PE/EA (2:3) to afford tert-butyl N-{2-[(5-formyl-2-hydroxyphenyl)formamido]ethyl}carbamate (66.4 g, 71%) as a white solid. LCMS (ESI, m/z):309[M+H]⁺

### Step 5: Synthesis of Compound 8

A solution of tert-butyl N-{2-[(5-formyl-2-hydroxyphenyl)formamido]ethyl}carbamate (61.8 g, 200.43 mmol, 1 equiv) in acetonitrile was treated with Ag₂O (92.89 g, 400.86 mmol, 2 equiv) at 0° C. under nitrogen atmosphere followed by the addition of methyl (2S,3S,4S,5R,6R)-3,4,5-tris(acetyloxy)-6-bromooxane-2-carboxylate (87.57 g, 397.17 mmol, 1.1 equiv) in portions at 0° C. for overnight. LCMC indicated the reaction was completed. The resulting mixture was filtered, the filter cake was washed with acetonitrile (3×10 mL). The filtrate was concentrated under reduced pressure. The residue was purified by silica gel column chromatography, eluted with PE/EA (1:4) to afford methyl (2S,3S,4S,5R,6S)-3,4,5-tris(acetyloxy)-6-[2-({2-[(tert-butoxycarbonyl)amino]ethyl}carbamoyl)-4-formylphenoxy]oxane-2-carboxylate (68.3 g, 54%) as an off-white solid. LCMS (ESI, m/z):625[M+H]⁺

### Step 6: Synthesis of Compound 9

To a stirred solution of methyl (2S,3S,4S,5R,6S)-3,4,5-tris(acetyloxy)-6-[2-({2-[(tert-butoxycarbonyl)amino]ethyl}carbamoyl)-4-formylphenoxy]oxane-2-carboxylate (20 g, 32.02 mmol, 1.00 equiv) in EA (200 mL) were added Pd/C (4.0 g, 37.58 mmol, 1.17 equiv, dry) in portions at room temperature. The resulting mixture was stirred for overnight at room temperature under hydrogen atmosphere. LCMS indicated the reaction was completed. The residue was purified by silica gel column chromatography, eluted with CH₂Cl₂/MeOH (19:1) to afford methyl (2S,3S,4S,5R,6S)-3,4,5-tris(acetyloxy)-6-[2-({2-[(tert-butoxycarbonyl)amino]ethyl}carbamoyl)-4-(hydroxymethyl)phenoxy]oxane-2-carboxylate (12.5 g, 58%) as a white solid. LCMS (ESI, m/z):627[M+H]⁺

### Step 7: Synthesis of Compound 10

To a stirred solution of 2-[6-(ethylamino)-4-[(1r,3s)-3-methyl-1-(4-methyl-1,2,4-triazol-3-yl)cyclobutyl]pyridin-2-yl]-6-{[(3S)-3-methylpiperidin-1-yl]methyl}-4-(methylsulfanyl)-3H-isoindol-1-one (400 mg, 0.71 mmol, 1 equiv) in THE (40 mL) was added Triphosgene (318 mg, 1.07 mmol, 1.5 equiv) in portions at room temperature under nitrogen atmosphere. The resulting mixture was stirred for 30 min at room temperature under nitrogen atmosphere. The resulting mixture was concentrated under vacuum. The residue was added to a stirred solution of methyl (2S,3S,4S,5R,6S)-3,4,5-tris(acetyloxy)-6-[2-({2-[(tert-butoxycarbonyl)amino]ethyl}carbamoyl)-4-(hydroxymethyl)phenoxy]oxane-2-carboxylate (672 mg, 1.07 mmol, 1.5 equiv) and TEA (217 mg, 2.14 mmol, 3 equiv) in DCM (40 mL) was stirred for 30 min at room temperature under nitrogen atmosphere. To the above mixture was added DMAP (174 mg, 1.43 mmol, 2 equiv) at room temperature. The resulting mixture was stirred for additional overnight at room temperature. LCMS showed 22% product. The resulting mixture was concentrated under vacuum. The residue was purified by reversed-phase flash chromatography with the following conditions: column, C18 silica gel; mobile phase, MeCN in Water (0.05% TFA), 5% to 60% gradient in 30 min; detector, UV 254 nm. The collected fraction was concentrated under vacuum and lyophilized.to afford methyl (2S,3S,4S,5R,6S)-3,4,5-tris(acetyloxy)-6-[2-({2-[(tert-butoxycarbonyl)amino]ethyl}carbamoyl)-4-[({ethyl[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamoyl}oxy)methyl]phenoxy]oxane-2-carboxylate (200 mg, 23%) as an off-white solid. LCMS (ESI, m/z):1212[M+H]⁺

### Step 8: Synthesis of Compound 11

To a stirred solution of methyl (2S,3S,4S,5R,6S)-3,4,5-tris(acetyloxy)-6-[2-({2-[(tert-butoxycarbonyl)amino]ethyl}carbamoyl)-4-[({ethyl[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamoyl}oxy)methyl]phenoxy]oxane-2-carboxylate (200 mg, 0.16 mmol, 1 equiv) in MeOH (5 mL) was added NaOMe (89 mg, 1.65 mmol, 10 equiv) at room temperature. The resulting mixture was stirred for 2 h at room temperature under nitrogen atmosphere. LCMS indicated the reaction was completed. The resulting mixture was concentrated under vacuum. This resulted in (2S,3S,4S,5R,6S)-6-[2-({2-[(tert-butoxycarbonyl)amino]ethyl}carbamoyl)-4-[({ethyl[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamoyl}oxy)methyl]phenoxy]-3,4,5-trihydroxyoxane-2-carboxylic acid (150 mg, 84%) as a light yellow solid. The crude product was used in the next step directly without further purification. LCMS (ESI, m/z):1072[M+H]⁺

### Step 9: Synthesis of Compound 12

To a stirred solution of (2S,3S,4S,5R,6S)-6-[2-({2-[(tert-butoxycarbonyl)amino]ethyl}carbamoyl)-4-[({ethyl[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamoyl}oxy)methyl]phenoxy]-3,4,5-trihydroxyoxane-2-carboxylic acid (150 mg, 0.14 mmol, 1 equiv) in DCM (2.8 mL) was added TFA (560 uL) dropwise at 0° C. under air atmosphere. The resulting mixture was stirred for 1 h at room temperature under air atmosphere. LCMS indicated the reaction was 60% product. The resulting mixture was concentrated under reduced pressure. to afford (2S,3S,4S,5R,6S)-6-{2-[(2-aminoethyl)carbamoyl]-4-[({ethyl[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamoyl}oxy)methyl]phenoxy}-3,4,5-trihydroxyoxane-2-carboxylic acid (120 mg, 88%) as a yellow solid. LCMS:(ES·m/z):972[M+H]⁺.

### Step 10: Synthesis of Compound 229

To a stirred solution of (2S,3S,4S,5R,6S)-6-{2-[(2-aminoethyl)carbamoyl]-4-[({ethyl[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamoyl}oxy)methyl]phenoxy}-3,4,5-trihydroxyoxane-2-carboxylic acid (80 mg, 0.08 mmol, 1 equiv) in DMF (1.6 mL) was added 2,5-dioxopyrrolidin-1-yl 6-(2,5-dioxopyrrol-1-yl)hexanoate (28 mg, 0.09 mmol, 1.1 equiv) and DIEA (32 mg, 0.24 mmol, 3 equiv) dropwise at 0° C. under air atmosphere. The resulting mixture was stirred for 1 h at room temperature under air atmosphere. LCMS indicated the reaction was 50% product. The crude product (90 mg) was purified by Prep-HPLC with the following conditions (Column: Xselect CSH Prep C18 C18 Column, 30*150 mm, 5 m; Mobile Phase A: Water (0.05% TFA), Mobile Phase B: ACN; Flow rate: 60 mL/min mL/min; Gradient: 9% B to 39% B in 20 min; Wave Length: 254 nm/220 nm; RT1 (min): 17.03. The collected fraction was lyophilized to afford (2S,3S,4S,5R,6S)-6-[2-({2-[6-(2,5-dioxopyrrol-1-yl)hexanamido]ethyl}carbamoyl)-4-[({ethyl[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamoyl}oxy)methyl]phenoxy]-3,4,5-trihydroxyoxane-2-carboxylic acid (13.3 mg, 12%) as a white solid. LCMS:(ES·m/z):1165[M+1]^{+ 1}HNMR (400 MHz, DMSO-d₆) δ 9.54 (br s, 1H), 8.44 (s, 1H), 8.30 (t, J=5.6 Hz, 1H), 8.11 (s, 1H), 7.86-7.84 (m, 2H), 7.74-7.69 (m, 2H), 7.56-7.53 (m, 2H), 7.26 (d, J=8.8 Hz, 1H), 6.99 (s, 2H), 5.19 (s, 2H), 5.14 (d, J=7.2 Hz, 1H), 4.94 (s, 2H), 4.41 (s, 2H), 3.99-3.97 (m, 3H), 3.92-3.93 (m, 3H), 3.46-3.25 (m, 9H), 3.22-3.16 (m, 6H), 2.84-2.70 (m, 2H), 2.64 (s, 3H), 2.55-2.52 (m, 1H), 2.04 (t, J=7.6 Hz, 2H), 1.84-1.64 (m, 4H), 1.48-1.42 (m, 4H), 1.30-1.23 (m, 3H), 1.17-1.13 (m, 2H), 1.06-1.03 (m, 4H), 0.88 (d, J=6.4 Hz, 3H).

### Synthesis of Compound 230

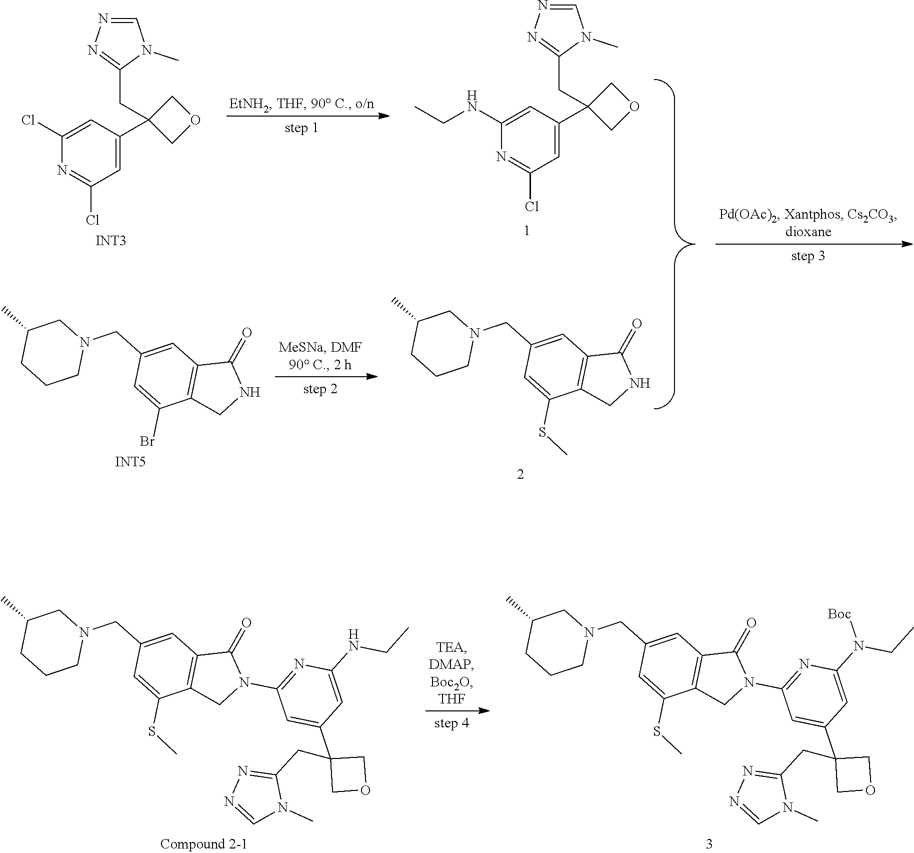

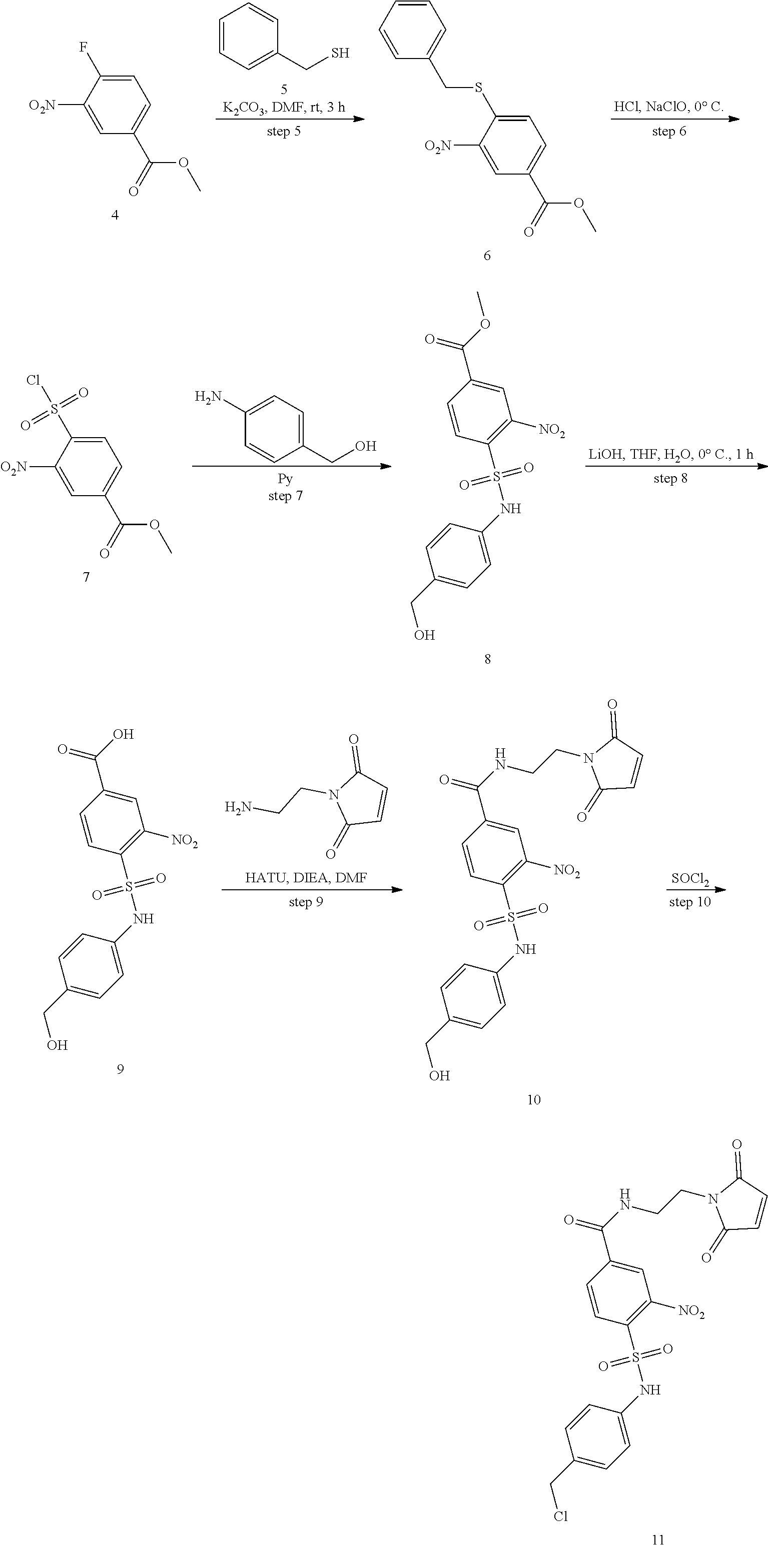

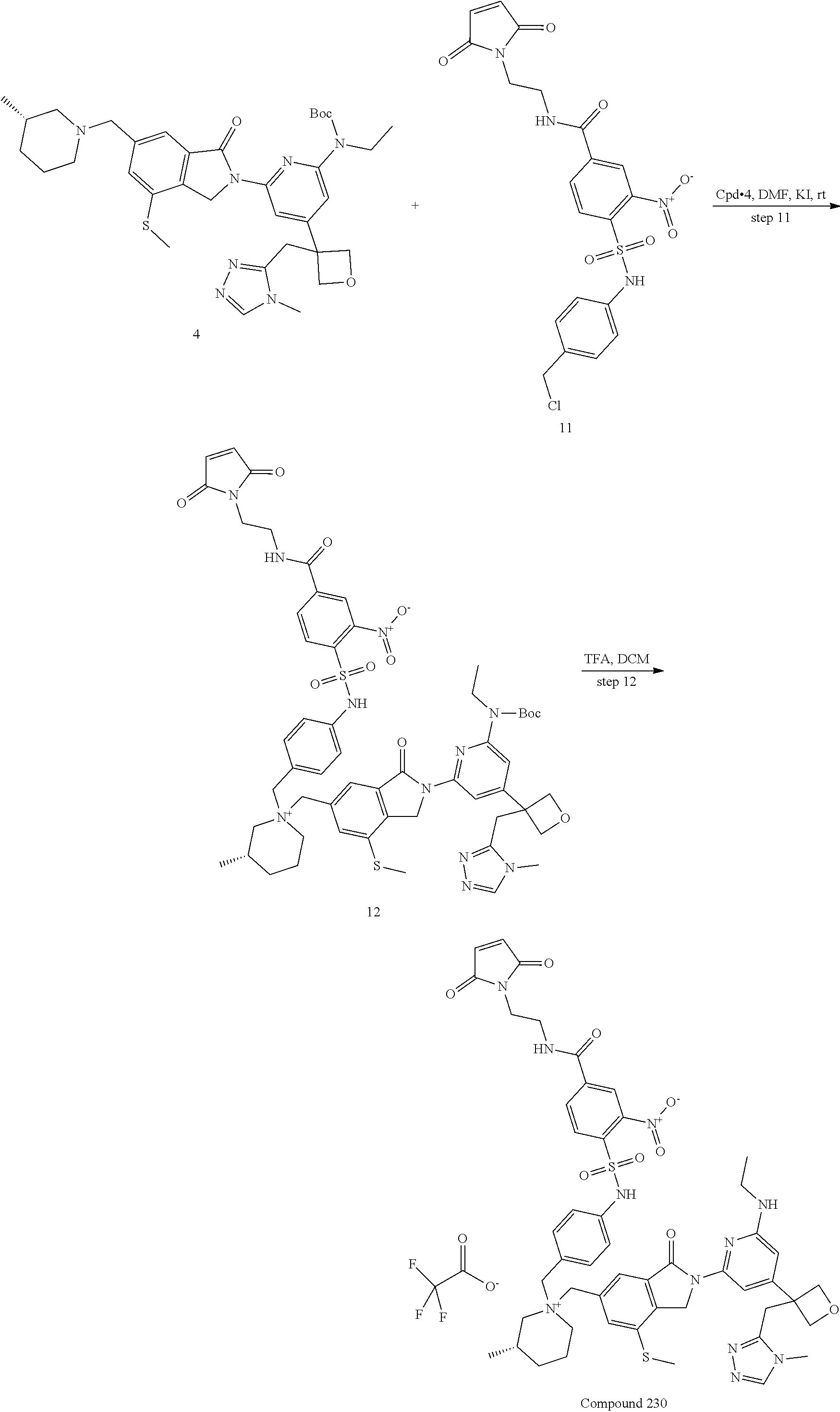 [SMILES: CCN(C(=O)OC(C)(C)C)c1cc(C2(Cc3nncn3C)COC2)cc(N2Cc3c(SC)cc(CN4CCC[C@H](C)C4)cc3C2=O)n1 ; O=C(NCCN1C(=O)C=CC1=O)c1ccc(S(=O)(=O)Nc2ccc(CCl)cc2)c([N+](=O)[O-])c1 ; CCN(C(=O)OC(C)(C)C)c1cc(C2(Cc3nncn3C)COC2)cc(N2Cc3c(SC)cc(C[N+]4(Cc5ccc(NS(=O)(=O)c6ccc(C(=O)NCCN7C(=O)C=CC7=O)cc6[N+](=O)[O-])cc5)CCC[C@H](C)C4)cc3C2=O)n1 ; O=C([O-])C(F)(F)F ; CCNc1cc(C2(Cc3nncn3C)COC2)cc(N2Cc3c(SC)cc(C[N+]4(Cc5ccc(NS(=O)(=O)c6ccc(C(=O)NCCN7C(=O)C=CC7=O)cc6[N+](=O)[O-])cc5)CCC[C@H](C)C4)cc3C2=O)n1]

### Step 1: Synthesis of Compound 1

To the solution of 4-bromo-6-{[(3S)-3-methylpiperidin-1-yl]methyl}-2,3-dihydroisoindol-1-one (1.5 g, 4.64 mmol, 1 equiv) in DMF (30 mL) was treated with (methylsulfanyl)sodium (488 mg, 6.96 mmol, 1.5 equiv) at 25° C. The solution was stirred for 1 h at 90° C. LCMS indicated the reaction was completed. The mixture was allowed to cool down to room temperature. The reaction mixture was purified by reverse flash chromatography with the following conditions: column, C18 silica gel, 80 g, 20-35 um; mobile phase, water with 8% NH₄HCO₃ and ACN (0% to 50% gradient in 50 min); detector, UV 254 nm. This resulted in 6-{[(3S)-3-methylpiperidin-1-yl]methyl}-4-(methylsulfanyl)-2,3-dihydroisoindol-1-one (670 mg, 50%) as a white solid. LCMS (ES, m/z): 291 [M+H]⁺

### Step 2: Synthesis of Compound 2

To a stirred solution of 2,6-dichloro-4-{3-[(4-methyl-1,2,4-triazol-3-yl)methyl]oxetan-3-yl}pyridine (800 mg, 2.67 mmol, 1 equiv) in Ethylamine solution (2.0 M in THF, 10 mL) was stirred for overnight at 90° C. under nitrogen atmosphere. LCMS indicated the reaction was completed. The mixture was allowed to cool down to room temperature and concentrated to dryness under vacuum. The residue was purified by silica gel column chromatography, eluted with CH₂Cl₂/MeOH (10:1) to afford 6-chloro-N-ethyl-4-{3-[(4-methyl-1,2,4-triazol-3-yl)methyl]oxetan-3-yl}pyridin-2-amine (450 mg, 54%) as an off-white solid. LCMS (ES, m/z): 308,310 [M+H]⁺

### Step 3: Synthesis of Compound 2-1

To a stirred solution of 6-chloro-N-ethyl-4-{3-[(4-methyl-1,2,4-triazol-3-yl)methyl]oxetan-3-yl}pyridin-2-amine (900 mg, 2.92 mmol, 1 equiv) and 6-{[(3S)-3-methylcyclohexyl]methyl}-4-(methylsulfanyl)-2,3-dihydroisoindol-1-one (846 mg, 2.92 mmol, 1 equiv) in dioxane (18 mL) was added Cs₂CO₃ (1905 mg, 5.84 mmol, 2 equiv), Xantphos (338 mg, 0.58 mmol, 0.2 equiv) and Pd(OAc)₂ (66 mg, 0.29 mmol, 0.1 equiv) at room temperature. The resulting mixture was stirred for 2 h at 120° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (10:1) to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one; trifluoroacetic acid (800 mg) as an off-white solid. The crude product (800 mg) was purified by Prep-HPLC with the following conditions (Column: Xselect CSH C18 OBD Column 30*150 mm 5 m, n; Mobile Phase A: Water (0.05% TFA), Mobile Phase B: ACN; Flow rate: 60 mL/min; Gradient: 10% B to 30% B in 9 min, 30% B; Wave Length: 254; 220 nm; RT1 (min): 8.65; The collected fraction was lyophilized to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one; trifluoroacetic acid (600 mg, 30%) as a yellow solid. LCMS (ES, m/z): 562 [M+H]⁺

### Step 4: Synthesis of Compound 3

To a stirred mixture of 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (250 mg, 0.44 mmol, 1 equiv) in THE (5 mL) were added TEA (90 mg, 0.89 mmol, 2 equiv), DMAP (16 mg, 0.13 mmol, 0.3 equiv) and Boc₂O (388 mg, 1.78 mmol, 4 equiv) in portions at room temperature. The resulting mixture was stirred for overnight at 55° C. The mixture was allowed to cool down to room temperature. LCMS indicated the reaction was completed. The resulting mixture was concentrated under reduced pressure. The residue was purified by silica gel column chromatography, eluted with PE/EA (1:1) to afford tert-butyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (250 mg, 85%) as a yellow solid. LCMS:(ES·m/z):662[M+H]⁺.

### Step 5: Synthesis of Compound 6

To a stirred mixture of methyl 4-fluoro-3-nitrobenzoate (25 g, 125.54 mmol, 1 equiv) and K₂CO₃ (34.70 g, 251.08 mmol, 2 equiv) in DMF (200 mL) was added benzyl mercaptan (31.18 g, 251.08 mmol, 2 equiv) in portions at 0° C. under nitrogen atmosphere. The resulting mixture was stirred for 3 h at room temperature under nitrogen atmosphere. LCMS indicated the reaction was completed. The reaction was quenched by the addition of Water/Ice (500 mL) at 0° C. The precipitated solids were collected by filtration and washed with PE (3×300 mL). The resulting solid was dried under infrared light. This resulted in methyl 4-(benzylsulfanyl)-3-nitrobenzoate (34 g, 89%) as a green solid. LCMS (ES, m/z): 304 [M+H]⁺

### Step 6: Synthesis of Compound 7

To a stirred solution of methyl 4-(benzylsulfanyl)-3-nitrobenzoate (25 g, 82.42 mmol, 1 equiv) in DCM (500 mL) was added HCl (aq, 6N, 500 mL) and NaClO (13%, 250 mL) in portions at 0° C. under nitrogen atmosphere. The resulting mixture was stirred for 1 h at 0° C. under nitrogen atmosphere. LCMS indicated the reaction was completed. The reaction was quenched with Water/Ice at 0° C. The resulting mixture was extracted with CH₂Cl₂ (3×500 mL). The combined organic layers were washed with brine (3×500 mL), dried over anhydrous Na₂SO₄. After filtration, the filtrate was concentrated under reduced pressure. The residue was purified by silica gel column chromatography, eluted with PE/EA (1:1) to afford methyl 4-(chlorosulfonyl)-3-nitrobenzoate (10 g, 43%) as a yellow solid. LCMS (ES, m/z): 260 [M−H-H₂O]⁻

### Step 7: Synthesis of Compound 8

To a stirred solution of β-aminobenzylalcohol (5.28 g, 42.91 mmol, 1.2 equiv) in DCM (100 mL) was added pyridine (5.66 g, 71.52 mmol, 2 equiv) and methyl 4-(chlorosulfonyl)-3-nitrobenzoate (10 g, 35.76 mmol, 1 equiv) dropwise at 0° C. under nitrogen atmosphere. The resulting mixture was stirred for 30 min at 0° C. under nitrogen atmosphere. LCMS indicated the reaction was completed. The reaction was quenched with Water/Ice at 0° C. The resulting mixture was extracted with CH₂Cl₂ (3×100 mL). The combined organic layers were washed with brine (3×100 mL), dried over anhydrous Na₂SO₄. After filtration, the filtrate was concentrated under reduced pressure. This resulted in methyl 4-{[4-(hydroxymethyl)phenyl]sulfamoyl}-3-nitrobenzoate (9 g, 68%) as a red solid. The crude product was used in the next step directly without further purification. LCMS (ES, m/z): 349 [M+H−H₂O]⁺

### Step 8: Synthesis of Compound 9

To a stirred solution of methyl 4-{[4-(hydroxymethyl)phenyl]sulfamoyl}-3-nitrobenzoate (7.5 g, 20.47 mmol, 1 equiv) in THE (750 mL) was added LiGH (0.98 g, 40.94 mmol, 2 equiv) in portions at 0° C. under nitrogen atmosphere. The resulting mixture was stirred for 1 h at 0° C. under nitrogen atmosphere. LCMS indicated the reaction was completed. The mixture was acidified to pH=3 with conc. HCl. The resulting mixture was purified by reversed-phase flash chromatography with the following conditions: column, C18 silica gel; mobile phase, ACN in Water (0.05% TFA), 5% to 60% gradient in 10 min; detector, UV 254 nm. The collected fraction was concentrated to afford 4-{[4-(hydroxymethyl)phenyl]sulfamoyl}-3-nitrobenzoic acid (3 g, 42%) as a red solid. LCMS (ES, m/z): 335 [M+H−H₂O]⁺

### Step 9: Synthesis of Compound 10

To a stirred solution of 4-{[4-(hydroxymethyl)phenyl]sulfamoyl}-3-nitrobenzoic acid (1.5 g, 4.25 mmol, 1 equiv) in DMF (15 mL) was added HATU (2.43 g, 6.38 mmol, 1.5 equiv) dropwise at 0° C. under nitrogen atmosphere. The resulting mixture was stirred for 30 min at 0° C. under nitrogen atmosphere. To the above mixture was added 1-(2-aminoethyl)pyrrole-2,5-dione (0.72 g, 5.108 mmol, 1.2 equiv) and DIEA(1.64 g, 12.75 mmol, 3 equiv) dropwise at 0° C. The resulting mixture was stirred for additional 2 h at room temperature. LCMS indicated the reaction was completed. The residue was purified by reversed-phase flash chromatography with the following conditions: column, C18 silica gel; mobile phase, ACN in Water (0.05% TFA), 5% to 60% gradient in 30 min; detector, UV 254 nm. The collected fraction was concentrated to afford N-[2-(2,5-dioxopyrrol-1-yl)ethyl]-4-{[4-(hydroxymethyl)phenyl]sulfamoyl}-3-nitrobenzamide (0.8 g, 40%) as a brown solid. LCMS (ES, m/z): 457 [M+H−H₂O]⁺

### Step 10: Synthesis of Compound 11

To a stirred mixture of N-[2-(2,5-dioxopyrrol-1-yl)ethyl]-4-{[4-(hydroxymethyl)phenyl]sulfamoyl}-3-nitrobenzamide (150 mg, 0.32 mmol, 1 equiv) and DMF (0.1 mL, 1.29 mmol, 4.09 equiv) in THE (3 mL) was added SOCl₂ (75 mg, 0.63 mmol, 2 equiv) in THE (0.05 mL) dropwise at 0° C. The resulting mixture was stirred at 0° C. for 30 min. LCMS indicated the reaction was completed. Then water (3 mL) was added into the above mixture and the resulting mixture was lyophilized to afford crude 4-{[4-(chloromethyl)phenyl]sulfamoyl}-N-[2-(2,5-dioxopyrrol-1-yl)ethyl]-3-nitrobenzamide (170 mg, crude) as a yellow solid. LCMS (ESI, m/z): 457 [M+H−HCl]⁺.

### Step 11: Synthesis of Compound 12

To a stirred solution of tert-butyl N-ethyl-N-(4-(3-[(4-methyl-1,2,4-triazol-3-yl)methyl]oxetan-3-yl-6-(6-([(3S)-3-methylpiperidin-1-yl]methyl-4-(methylsulfanyl)-1-oxo-3H-isoindol-2-yl)pyridin-2-yl)carbamate (175 mg, 0.26 mmol, 1 equiv) and 4-([4-(chloromethyl)phenyl]sulfamoyl-N-[2-(2,5-dioxopyrrol-1-yl)ethyl]-3-nitrobenzamide (156 mg, 0.31 mmol, 1.2 equiv) in DMF (5 mL) was added KI (88 mg, 0.52 mmol, 2 equiv) at room temperature. The resulting mixture was stirred for 2 days at room temperature under nitrogen atmosphere. LCMS indicated the reaction was completed. The mixture was purified by reversed-phase flash chromatography with the following conditions: column, C18 silica gel; mobile phase, MeCN in Water (0.1% TFA), 5% to 80% gradient in 30 min; detector, UV 254 nm. The collected fraction was concentrated under vacuum and lyophilized to afford (3S)-1-[(2-(6-[(tert-butoxycarbonyl)(ethyl)amino]-4-(3-[(4-methyl-1,2,4-triazol-3-yl)methyl]oxetan-3-ylpyridin-2-yl-7-(methylsulfanyl)-3-oxo-1H-isoindol-5-yl)methyl]-1-([4-(4-([2-(2,5-dioxopyrrol-1-yl)ethyl]carbamoyl-2-nitrobenzenesulfonamido)phenyl]methyl-3-methylpiperidin-1-ium (100 mg, 34%) as a white solid. LCMS (ESI, m/z):1118[M]⁺

### Step 12: Synthesis of Compound 230

To a stirred solution of (3S)-1-[(2-(6-[(tert-butoxycarbonyl)(ethyl)amino]-4-(3-[(4-methyl-1,2,4-triazol-3-yl)methyl]oxetan-3-ylpyridin-2-yl-7-(methylsulfanyl)-3-oxo-1H-isoindol-5-yl)methyl]-1-([4-(4-([2-(2,5-dioxopyrrol-1-yl)ethyl]carbamoyl-2-nitrobenzenesulfonamido)phenyl]methyl-3-methylpiperidin-1-ium (100 mg, 0.08 mmol, 1 equiv) in DCM (3 mL) was added TFA (1.2 mL) dropwise at 0° C. The resulting mixture was stirred for 1 h at 0° C. under nitrogen atmosphere. LCMS indicated the reaction was completed. The resulting mixture was concentrated under vacuum. The crude product (50 mg) was purified by Prep-HPLC with the following conditions (Column: XSelect CSH Prep C18 OBD Column, 19*150 mm, 5 m; Mobile Phase A: Water (0.05% TFA), Mobile Phase B: ACN; Flow rate: 25 mL/min; Gradient: 17% B to 47% B in 7 min; Wave Length: 254 nm; RT1 (min): 5.15. The collected fraction was lyophilized to afford (3S)-1-([4-(4-([2-(2,5-dioxopyrrol-1-yl)ethyl]carbamoyl-2-nitrobenzenesulfonamido)phenyl]methyl-1-((2-[6-(ethylamino)-4-(3-[(4-methyl-1,2,4-triazol-3-yl)methyl]oxetan-3-ylpyridin-2-yl]-7-(methylsulfanyl)-3-oxo-1H-isoindol-5-ylmethyl)-3-methylpiperidin-1-ium; trifluoroacetic acid (12.7 mg, 12%) as a white solid. LCMS (ESI, m/z):1018[M+H−TFA]^{+ 1}H NMR (400 MHz, DMSO-d₆) δ 11.08 (s, 1H), 9.89 (s, 2H), 9.04 (t, J=6.0 Hz, 1H), 8.25-8.08 (m, 3H), 7.71-7.50 (m, 2H), 7.37-7.28 (m, 3H), 7.22-7.08 (m, 2H), 7.01 (s, 2H), 6.12 (s, 1H), 5.47 (s, 2H), 4.91 (s, 2H), 4.84-4.71 (m, 4H), 4.43 (s, 2H), 3.72 (s, 2H), 3.60-3.56 (m, 6H), 3.39-3.22 (m, 6H), 2.87-2.77 (m, 1H), 2.58 (s, 3H), 1.84-1.64 (m, 4H), 1.16 (t, J=6.8 Hz, 3H), 1.10-0.99 (m, 1H), 0.88 (d, J=6.4 Hz, 3H).

### Synthesis of Compound 231

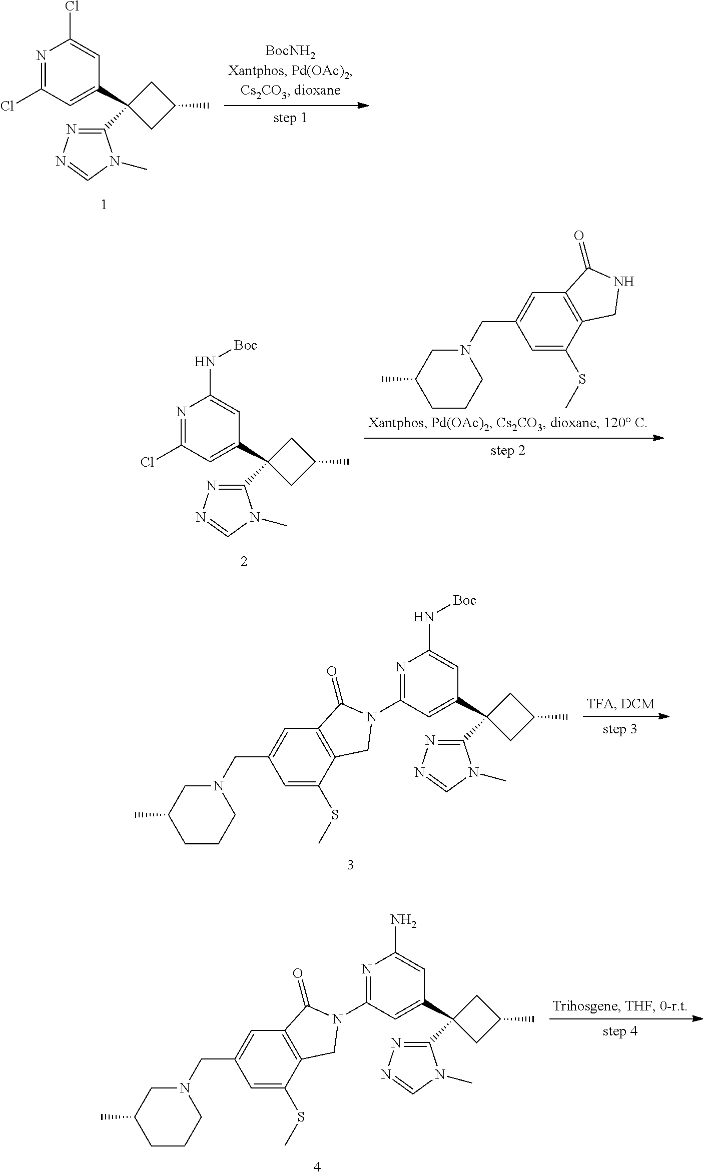

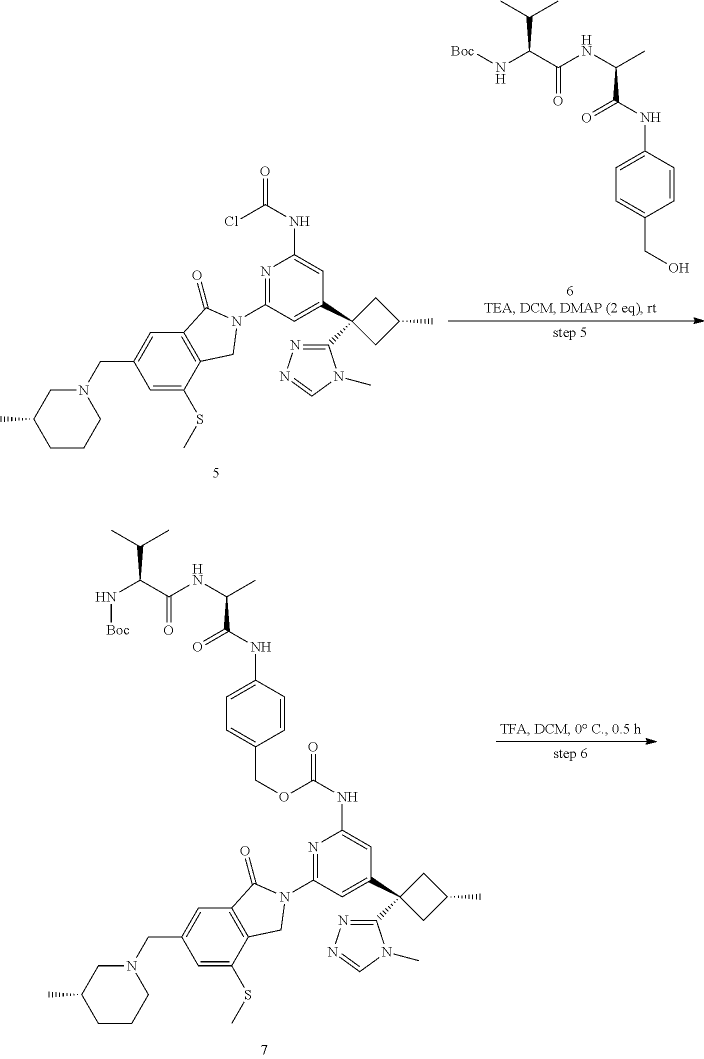

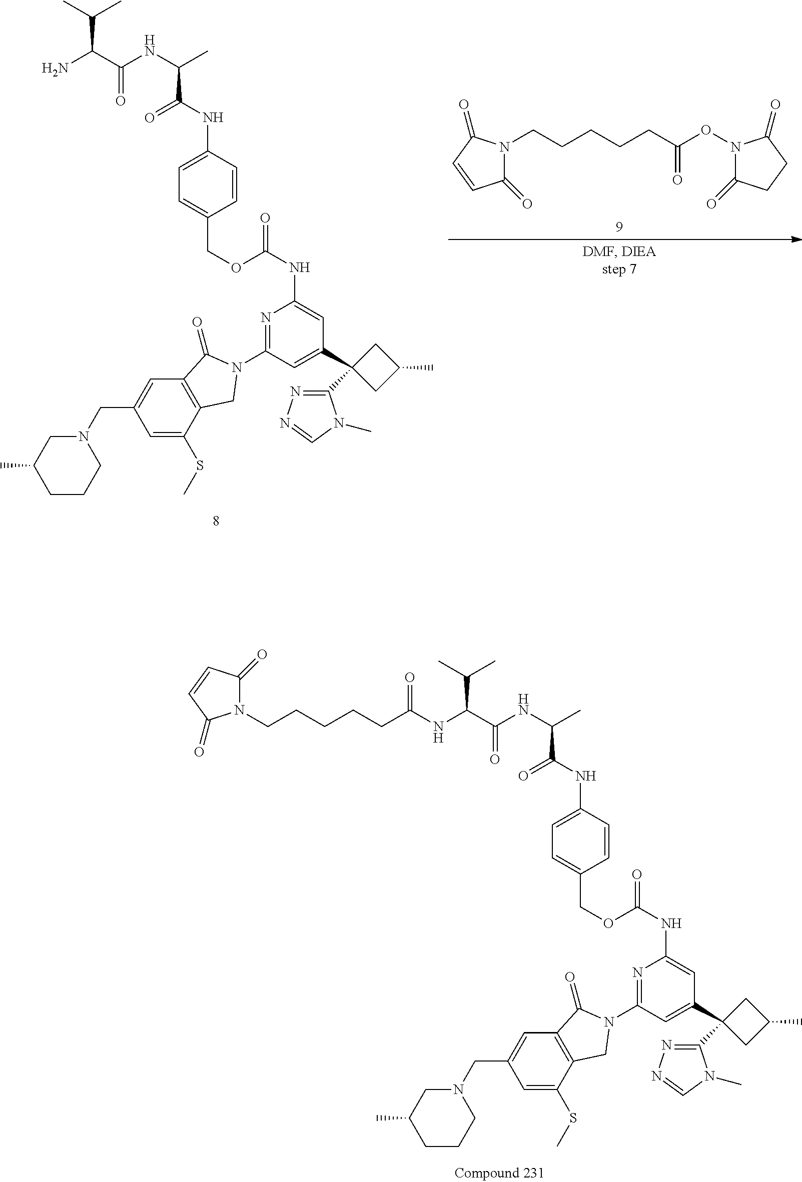

### Step 1: Synthesis of Compound 2

A mixture of 2,6-dichloro-4-[(1r,3s)-3-methyl-1-(4-methyl-1,2,4-triazol-3-yl)cyclobutyl]pyridine (800 mg, 2.69 mmol, 1 equiv), BocNH₂ (315 mg, 2.69 mmol, 1 equiv), XantPhos (311 mg, 0.54 mmol, 0.2 equiv), Pd(OAc)₂ (120 mg, 0.54 mmol, 0.2 equiv) and Cs₂CO₃ (1.75 g, 5.38 mmol, 2 equiv) in dioxane (16 mL) was stirred for 1 h at 90° C. under nitrogen atmosphere. LCMS indicated the reaction was completed. The reaction mixture was allowed to cool down to room temperature and the solvent was removed under reduced pressure. The residue was purified by silica gel column chromatography, eluted with DCM/MeOH (94:6) to afford tert-butyl N-{6-chloro-4-[(1r,3s)-3-methyl-1-(4-methyl-1,2,4-triazol-3-yl)cyclobutyl]pyridin-2-yl}carbamate (510 mg, 50%) as a yellow solid. LCMS (ESI, m/z): 378,380 [M+H]⁺.

### Step 2: Synthesis of Compound 3

A mixture of tert-butyl N-{6-chloro-4-[(1r,3s)-3-methyl-1-(4-methyl-1,2,4-triazol-3-yl)cyclobutyl]pyridin-2-yl}carbamate (710 mg, 1.88 mmol, 1 equiv), 6-{[(3S)-3-methylpiperidin-1-yl]methyl}-4-(methylsulfanyl)-2,3-dihydroisoindol-1-one (545 mg, 1.88 mmol, 1 equiv), Pd(OAc)₂ (84 mg, 0.37 mmol, 0.2 equiv), XantPhos (217 mg, 0.37 mmol, 0.2 equiv) and Cs₂CO₃ (122 mg, 0.37 mmol, 0.2 equiv) in dioxane (15 mL) was stirred for 1 h at 120° C. under nitrogen atmosphere. LCMS indicated the reaction was completed. The reaction mixture was allowed to cool down to room temperature and the solvent was removed under reduced pressure. The residue was purified by silica gel column chromatography, eluted with DCM/MeOH (95:5) to afford tert-butyl N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate (540 mg, 45%) as a yellow soild. LCMS (ESI, m/z): 632 [M+H]⁺.

### Step 3: Synthesis of Compound 4

To a mixture of tert-butyl N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate (440 mg, 0.70 mmol, 1 equiv) in DCM (8 mL) was added TFA (1.6 mL) at 25° C. The resulting mixture was stirred for 1 h at 25° C. LCMS indicated the reaction was completed. The solvent was removed under reduced pressure. The residue was purified by reverse flash chromatography with the following conditions: column, C18 silica gel, 80 g, 20-35 um; mobile phase, water with 0.1% NH₄HCO₃ and ACN (0% to 100% gradient in 50 min); detector, UV 254 nm. The collected fraction was lyophilized to afford 2-{6-amino-4-[(1r,3s)-3-methyl-1-(4-methyl-1,2,4-triazol-3-yl)cyclobutyl]pyridin-2-yl}-6-{[(3 S)-3-methylpiperidin-1-yl]methyl}-4-(methylsulfanyl)-3H-isoindol-1-one (300 mg, 81%) as a white solid. LCMS (ESI, m/z): 532 [M+H]⁺.

### Step 4: Synthesis of Compound 5

To a mixture of 2-{6-amino-4-[(1r,3s)-3-methyl-1-(4-methyl-1,2,4-triazol-3-yl)cyclobutyl]pyridin-2-yl}-6-{[(3S)-3-methylpiperidin-1-yl]methyl}-4-(methylsulfanyl)-3H-isoindol-1-one (50 mg, 0.094 mmol, 1 equiv) in THE (4.7 mL) was added triphosgene (70 mg, 0.235 mmol, 2.5 equiv) at 25° C. under nitrogen atmosphere. The resulting mixture was stirred for 1 h at 25° C. under nitrogen atmosphere. LCMS indicated the reaction was completed. The solvent was removed under reduced pressure at 5-10° C. to afford crude N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamoyl chloride (60 mg, 107%) as a white solid. The residue was used for next step without further purification. No mass signal.

### Step 5: Synthesis of Compound 7

A mixture of tert-butyl N-[(1S)-1-{[(1S)-1-{[4-(hydroxymethyl)phenyl]carbamoyl}ethyl]carbamoyl}-2-methylpropyl]carbamate (40 mg, 0.10 mmol, 1 equiv) and TEA (30 mg, 0.30 mmol, 3 equiv) in DCM (1.5 mL) was stirred for 10 min. The resulting mixture was added into N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamoyl chloride (60 mg, 0.10 mmol, 1 equiv) in DCM (1.5 mL). The resulting mixture was stirred for 1 h at 25° C. LCMS indicated 33% product. The solvent was removed under reduced pressure. The residue was purified by reverse flash chromatography with the following conditions: column, C18 silica gel, 80 g, 20-35 um; mobile phase, water with 0.1% TFA and ACN (0% to 100% gradient in 50 min); detector, UV 254 nm. The reaction was run four times in parallel. Combined with other four batches, the collected fraction was concentrated to afford {4-[(2S)-2-[(2S)-2-amino-3-methylbutanamido]propanamido]phenyl}methyl N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate (30 mg, 6% for two steps) as a yellow solid. LCMS (ESI, m/z): 951 [M+H]⁺.

### Step 6: Synthesis of Compound 8

A mixture of {4-[(2S)-2-[(2S)-2-amino-3-methylbutanamido]propanamido]phenyl}methyl N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate (30 mg, 0.031 mmol, 1 equiv) in DCM (2.5 mL) was added TFA (0.5 mL) at 25° C. The resulting mixture was stirred for 1 h at 25° C. LCMS indicated the reaction was completed. The solvent was removed under reduced pressure. The residue was lyophilized to afford crude {4-[(2S)-2-[(2S)-2-amino-3-methylbutanamido]propanamido]phenyl}methyl N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate (25 mg) as a brown solid. LCMS (ESI, m/z): 851 [M+H]⁺.

### Step 7: Synthesis of Compound 8

To a stirred solution of {4-[(2S)-2-[(2S)-2-amino-3-methylbutanamido]propanamido]phenyl}methyl N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate (26 mg, 0.031 mmol, 1 equiv) and 2,5-dioxopyrrolidin-1-yl 6-(2,5-dioxopyrrol-1-yl)hexanoate (11 mg, 0.037 mmol, 1.2 equiv) in DMF (1 mL) was added DIEA (8 mg, 0.062 mmol, 2 equiv) dropwise at 0° C. The resulting mixture was stirred for 30 min at 0°. LCMS indicated the reaction was completed. The reaction mixture was purified by Prep-HPLC with following conditions: Column: XBridge Prep Phenyl OBD Column, 19*250 mm, 5 m; Mobile Phase A: Water (0.05% TFA), Mobile Phase B: ACN; Flow rate: 25 mL/min; Gradient: 30% B to 50% B in 15 min; Wave Length: 254 nm; RT1 (min): 11.95. The collected fraction was lyophilized to afford {4-[(2S)-2-[(2S)-2-[6-(2,5-dioxopyrrol-1-yl)hexanamido]-3-methylbutanamido]propanamido]phenyl}methyl N-[6-(6-{[(3S)-3-methylpiperidin-1-yl]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]carbamate) as a white solid. LCMS (ES, m/z): 523[M+H]/2, 1067 [M+Na]⁺. ¹H NMR (400 MHz, DMSO-d₆) δ 10.38 (s, 1H), 9.99 (s, 1H), 9.71-8.57 (m, 1H), 8.47 (s, 1H), 8.18 (d, J=6.8 Hz, 1H), 8.11 (s, 1H), 7.83 (d, J=8.4 Hz, 1H), 7.70 (d, J=5.2 Hz, 2H), 7.66-7.60 (m, 3H), 7.37 (d, J=8.4 Hz, 2H), 6.98 (s, 2H), 5.12 (s, 2H), 4.91 (s, 2H), 4.46-4.34 (m, 3H), 4.17 (t, J=8.0 Hz, 1H), 3.36 (t, J=8.0 Hz, 2H), 3.24 (s, 3H), 2.84-2.77 (m, 2H), 2.65-2.55 (m, 5H), 2.22-2.14 (m, 3H), 1.98-1.92 (m, 1H), 1.88-1.63 (m, 5H), 1.52-1.40 (m, 4H), 1.3-5-1.28 (m, 4H), 1.25-1.15 (m, 4H), 1.15-1.05 (m, 4H), 0.90-0.85 (m, 6H), 0.82-0.81 (m, 3H).

### Synthesis of Compound 232

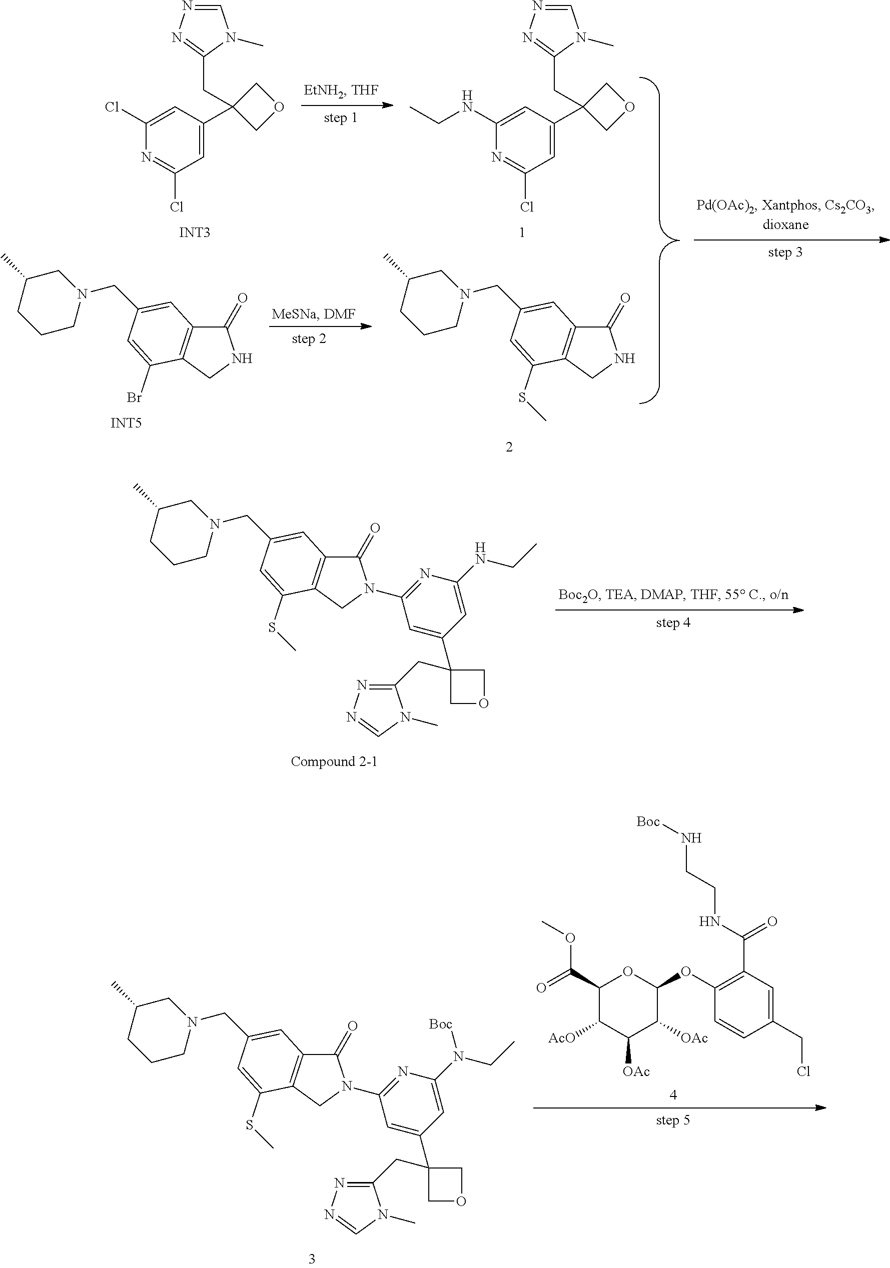

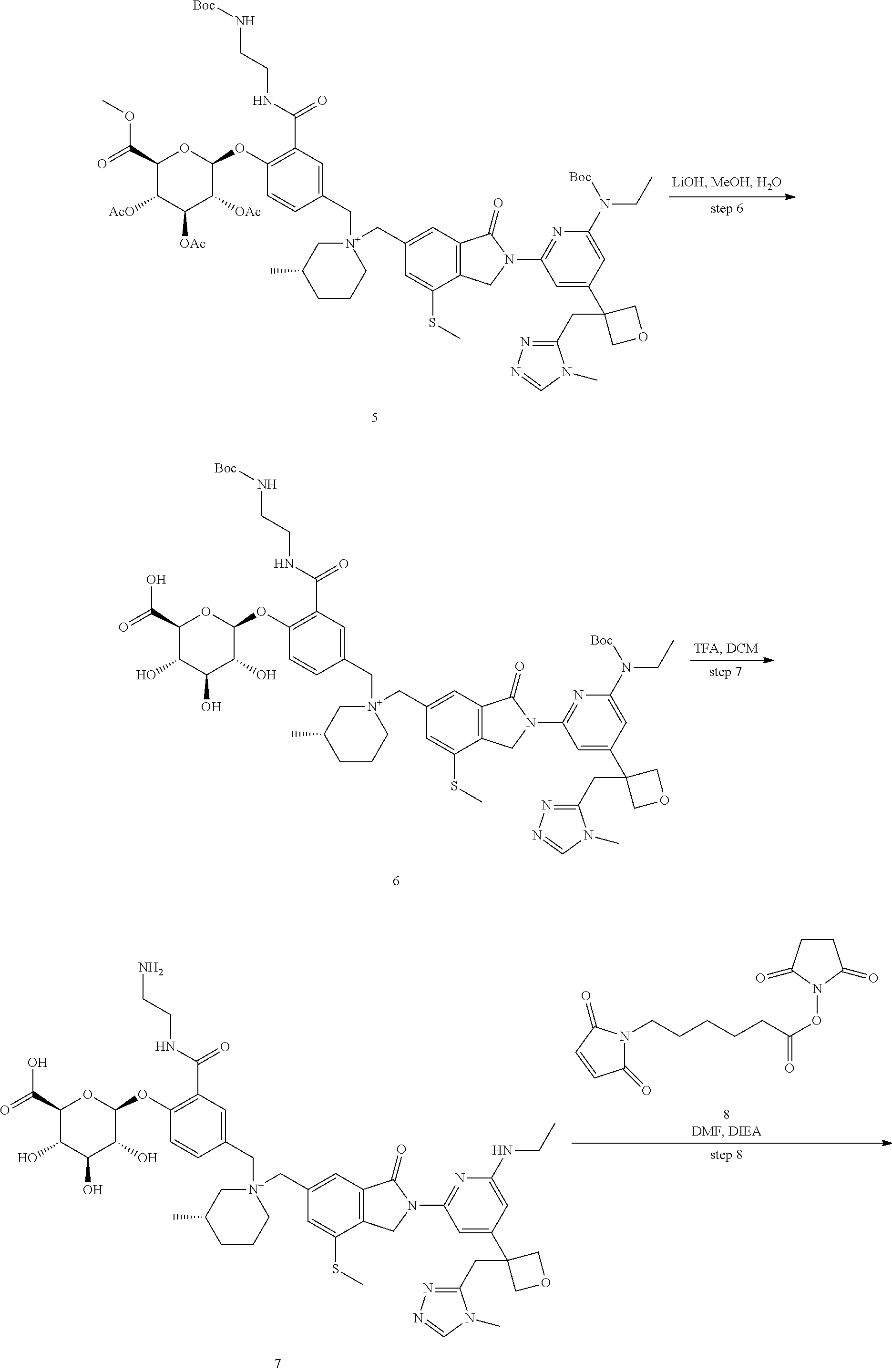

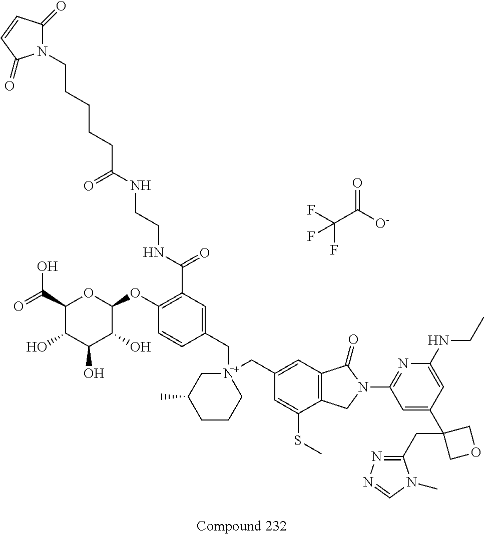 [SMILES: CCNc1cc(C2(Cc3nncn3C)COC2)cc(N2Cc3c(SC)cc(C[N+]4(Cc5ccc(O[C@@H]6O[C@H](C(=O)O)[C@@H](O)[C@H](O)[C@H]6O)c(C(=O)NCCNC(=O)CCCCCN6C(=O)C=CC6=O)c5)CCC[C@H](C)C4)cc3C2=O)n1 ; O=C([O-])C(F)(F)F]

### Step 1: Synthesis of Compound 1

To the solution of 4-bromo-6-{[(3S)-3-methylpiperidin-1-yl]methyl}-2,3-dihydroisoindol-1-one (1.5 g, 4.64 mmol, 1 equiv) in DMF (30 mL) was treated with (methylsulfanyl)sodium (488 mg, 6.96 mmol, 1.5 equiv) at 25° C. The solution was stirred for 1 h at 90° C. LCMS indicated the reaction was completed. The mixture was allowed to cool down to room temperature. The reaction mixture was purified by reverse flash chromatography with the following conditions: column, C18 silica gel, 80 g, 20-35 um; mobile phase, water with 8% NH₄HCO₃ and ACN (0% to 50% gradient in 50 min); detector, UV 254 nm. This resulted in 6-{[(3S)-3-methylpiperidin-1-yl]methyl}-4-(methylsulfanyl)-2,3-dihydroisoindol-1-one (670 mg, 50%) as a white solid. LCMS (ES, m/z): 291 [M+H]⁺

### Step 2: Synthesis of Compound 2

To a stirred solution of 2,6-dichloro-4-{3-[(4-methyl-1,2,4-triazol-3-yl)methyl]oxetan-3-yl}pyridine (800 mg, 2.67 mmol, 1 equiv) in Ethylamine solution (2.0 M in THF, 10 mL) was stirred for overnight at 90° C. under nitrogen atmosphere. LCMS indicated the reaction was completed. The mixture was allowed to cool down to room temperature and concentrated to dryness under vacuum. The residue was purified by silica gel column chromatography, eluted with CH₂Cl₂/MeOH (10:1) to afford 6-chloro-N-ethyl-4-{3-[(4-methyl-1,2,4-triazol-3-yl)methyl]oxetan-3-yl}pyridin-2-amine (450 mg, 54%) as an off-white solid. LCMS (ES, m/z): 308,310 [M+H]⁺

### Step 3: Synthesis of Compound 2-1

To a stirred solution of 6-chloro-N-ethyl-4-{3-[(4-methyl-1,2,4-triazol-3-yl)methyl]oxetan-3-yl}pyridin-2-amine (900 mg, 2.92 mmol, 1 equiv) and 6-{[(3S)-3-methylcyclohexyl]methyl}-4-(methylsulfanyl)-2,3-dihydroisoindol-1-one (846 mg, 2.92 mmol, 1 equiv) in dioxane (18 mL) was added Cs₂CO₃ (1905 mg, 5.84 mmol, 2 equiv), Xantphos (338 mg, 0.58 mmol, 0.2 equiv) and Pd(OAc)₂ (66 mg, 0.29 mmol, 0.1 equiv) at room temperature. The resulting mixture was stirred for 2 h at 120° C. under nitrogen atmosphere. LCMS indicated the reaction was completed. The mixture was allowed to cool down to room temperature. The resulting mixture was concentrated under vacuum. The residue was purified by silica gel column chromatography, eluted with CH₂Cl₂/MeOH (10:1) to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one; trifluoroacetic acid (800 mg) as an off-white solid. The crude product (800 mg) was purified by Prep-HPLC with the following conditions (Column: Xselect CSH C18 OBD Column 30*150 mm 5 m, n; Mobile Phase A: Water (0.05% TFA), Mobile Phase B: ACN; Flow rate: 60 mL/min; Gradient: 10% B to 30% B in 9 min, 30% B; Wave Length: 254; 220 nm; RT1 (min): 8.65; The collected fraction was lyophilized to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one; trifluoroacetic acid (600 mg, 30%) as a yellow solid. LCMS (ES, m/z): 562 [M+H]⁺

### Step 4: Synthesis of Compound 3

To a stirred mixture of 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (250 mg, 0.44 mmol, 1 equiv) in THE (5 mL) were added TEA (90 mg, 0.89 mmol, 2 equiv), DMAP (16 mg, 0.13 mmol, 0.3 equiv) and Boc₂O (388 mg, 1.78 mmol, 4 equiv) in portions at room temperature. The resulting mixture was stirred for overnight at 55° C. The mixture was allowed to cool down to room temperature. LCMS indicated the reaction was completed. The resulting mixture was concentrated under reduced pressure. The residue was purified by silica gel column chromatography, eluted with PE/EA (1:1) to afford tert-butyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (250 mg, 85%) as a yellow solid. LCMS:(ES·m/z):662[M+H]⁺.

### Step 5: Synthesis of Compound 5

A mixture of tert-butyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (600 mg, 0.91 mmol, 1 equiv), methyl (2S,3S,4S,5R,6S)-3,4,5-tris(acetyloxy)-6-[2-({2-[(tert-butoxycarbonyl)amino]ethyl}carbamoyl)-4-(chloromethyl)phenoxy]oxane-2-carboxylate (584 mg, 0.91 mmol, 1 equiv) and KI (300 mg, 1.81 mmol, 2 equiv) in acetone (24 mL) was stirred for 16 h at 50° C. LCMS indicated the reaction was completed. The solvent was removed under reduced pressure. The residue was purified by reverse flash chromatography with the following conditions: column, C18 silica gel, 80 g, 20-35 um; mobile phase, water with 0.1% TFA and ACN (0% to 100% gradient in 50 min); detector, UV 254 nm. The collected fraction was lyophilized to afford (3S)-1-[(2-{6-[(tert-butoxycarbonyl)(ethyl)amino]-4-{3-[(4-methyl-1,2,4-triazol-3-yl)methyl]oxetan-3-yl}pyridin-2-yl}-7-(methylsulfanyl)-3-oxo-1H-isoindol-5-yl)methyl]-1-{[3-({2-[(tert-butoxycarbonyl)amino]ethyl}carbamoyl)-4-{[(2S,3R,4S,5S,6S)-3,4,5-tris(acetyloxy)-6-(methoxycarbonyl)oxan-2-yl]oxy}phenyl]methyl}-3-methylpiperidin-1-ium (215 mg, 19%) as a yellow solid. LCMS (ESI, m/z): 1170 [M]⁺.

### Step 6: Synthesis of Compound 6

A mixture of (3S)-1-[(2-{6-[(tert-butoxycarbonyl)(ethyl)amino]-4-{3-[(4-methyl-1,2,4-triazol-3-yl)methyl]oxetan-3-yl}pyridin-2-yl}-7-(methylsulfanyl)-3-oxo-1H-isoindol-5-yl)methyl]-1-{[3-({2-[(tert-butoxycarbonyl)amino]ethyl}carbamoyl)-4-{[(2S,3R,4S,5S,6S)-3,4,5-tris(acetyloxy)-6-(methoxycarbonyl)oxan-2-yl]oxy}phenyl]methyl}-3-methylpiperidin-1-ium (200 mg, 0.16 mmol, 1 equiv) and LiGH (22 mg, 0.94 mmol, 6 equiv) in MeOH (3 mL) was stirred for 1 h at 25° C. LCMS indicated the reaction was completed. The solvent was removed under reduced pressure. The residue was purified by reverse flash chromatography with the following conditions: column, C18 silica gel, 80 g, 20-35 um; mobile phase, water with 0.1% TFA and ACN (0% to 100% gradient in 50 min); detector, UV 254 nm. The collected fraction was lyophilized to afford (3S)-1-[(2-{6-[(tert-butoxycarbonyl)(ethyl)amino]-4-{3-[(4-methyl-1,2,4-triazol-3-yl)methyl]oxetan-3-yl}pyridin-2-yl}-7-(methylsulfanyl)-3-oxo-1H-isoindol-5-yl)methyl]-1-{[3-({2-[(tert-butoxycarbonyl)amino]ethyl}carbamoyl)-4-{[(2S,3R,4S,5S,6S)-6-carboxy-3,4,5-trihydroxyoxan-2-yl]oxy}phenyl]methyl}-3-methylpiperidin-1-ium (105 mg, 59%) as a yellow solid. LCMS (ESI, m/z): 1130 [M]⁺.

### Step 7: Synthesis of Compound 7

To a mixture of (3S)-1-[(2-{6-[(tert-butoxycarbonyl)(ethyl)amino]-4-{3-[(4-methyl-1,2,4-triazol-3-yl)methyl]oxetan-3-yl}pyridin-2-yl}-7-(methylsulfanyl)-3-oxo-1H-isoindol-5-yl)methyl]-1-{[3-({2-[(tert-butoxycarbonyl)amino]ethyl}carbamoyl)-4-{[(2S,3R,4S,5S,6S)-6-carboxy-3,4,5-trihydroxyoxan-2-yl]oxy}phenyl]methyl}-3-methylpiperidin-1-ium (100 mg, 0.088 mmol, 1 equiv) in DCM (3 mL) was added TFA (0.6 mL) at 25° C. The resulting mixture was stirred at 25° C. for 1 h. LCMS indicated the reaction was completed. The solvent was removed under reduced pressure. The residue was lyophilized to afford crude (3S)-1-({3-[(2-aminoethyl)carbamoyl]-4-{[(2S,3R,4S,5S,6S)-6-carboxy-3,4,5-trihydroxyoxan-2-yl]oxy}phenyl}methyl)-1-({2-[6-(ethylamino)-4-{3-[(4-methyl-1,2,4-triazol-3-yl)methyl]oxetan-3-yl}pyridin-2-yl]-7-(methylsulfanyl)-3-oxo-1H-isoindol-5-yl}methyl)-3-methylpiperidin-1-ium (110 mg, crude) as a yellow solid. LCMS (ESI, m/z): 930 [M]⁺.

### Step 8: Synthesis of Compound 232

To a mixture of (3S)-1-({3-[(2-aminoethyl)carbamoyl]-4-{[(2S,3R,4S,5S,6S)-6-carboxy-3,4,5-trihydroxyoxan-2-yl]oxy}phenyl}methyl)-1-({2-[6-(ethylamino)-4-{3-[(4-methyl-1,2,4-triazol-3-yl)methyl]oxetan-3-yl}pyridin-2-yl]-7-(methylsulfanyl)-3-oxo-1H-isoindol-5-yl}methyl)-3-methylpiperidin-1-ium (80 mg, 0.086 mmol, 1 equiv) and DIEA (55 mg, 0.430 mmol, 5 equiv) in DMF (2 mL) was added 2,5-dioxopyrrolidin-1-yl 6-(2,5-dioxopyrrol-1-yl)hexanoate (39 mg, 0.129 mmol, 1.5 equiv) at 25° C. The resulting mixture was stirred for 1 h at 25° C. LCMS indicated the reaction was completed. The reaction mixture was purified with following conditions: Column: XBridge Prep OBD C18 Column, 19*250 mm, 5 m; Mobile Phase A: Water (0.1% TFA), Mobile Phase B: ACN; Flow rate: 25 mL/min; Gradient: 17% B to 35% B in 10 min, 35% B; Wave Length: 254 nm; RT1 (min): 9; Injection Volume: 0.3 mL; Number Of Runs: 5. The collected fraction was lyophilized to afford (3S)-1-[(4-{[(2S,3R,4S,5S,6S)-6-carboxy-3,4,5-trihydroxyoxan-2-yl]oxy}-3-({2-[6-(2,5-dioxopyrrol-1-yl)hexanamido]ethyl}carbamoyl)phenyl)methyl]-1-({2-[6-(ethylamino)-4-{3-[(4-methyl-1,2,4-triazol-3-yl)methyl]oxetan-3-yl}pyridin-2-yl]-7-(methylsulfanyl)-3-oxo-1H-isoindol-5-yl}methyl)-3-methylpiperidin-1-ium (3.6 mg, 2.07%) as a yellow semi-solid. LCMS (ESI, m/z): 1123 [M−TFA]⁺. ¹H NMR (400 MHz, Methanol-d4) δ 7.96 (s, 1H), 7.77 (s, 1H), 7.70 (s, 1H), 7.59 (dd, J=8.6, 2.4 Hz, 1H), 7.38 (d, J=8.6 Hz, 1H), 6.92 (s, 1H), 6.79 (s, 2H), 6.14 (s, 1H), 5.61 (s, 2H), 5.10 (d, J=7.4 Hz, 1H), 5.03-4.83 (m, 8H), 4.51 (s, 2H), 4.04 (d, J=9.6 Hz, 1H), 3.81 (s, 2H), 3.71-3.34 (m, 15H), 2.97-2.90 (m, 1H), 2.74-2.64 (m, 4H), 2.18 (t, J=7.4 Hz, 2H), 2.00-1.75 (m, 4H), 1.66-1.46 (m, 4H), 1.30-1.20 (m, 6H), 1.01 (d, J=6.3 Hz, 3H).

### Synthesis of Compound 233

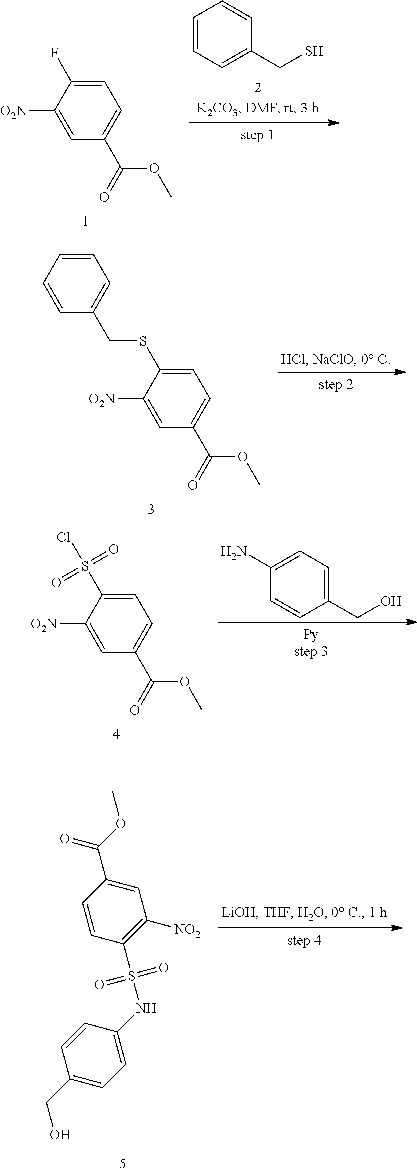 [SMILES: COC(=O)c1ccc(F)c([N+](=O)[O-])c1 ; SCc1ccccc1 ; COC(=O)c1ccc(SCc2ccccc2)c([N+](=O)[O-])c1 ; COC(=O)c1ccc(S(=O)(=O)Cl)c([N+](=O)[O-])c1 ; Nc1ccc(CO)cc1 ; COC(=O)c1ccc(S(=O)(=O)Nc2ccc(CO)cc2)c([N+](=O)[O-])c1]

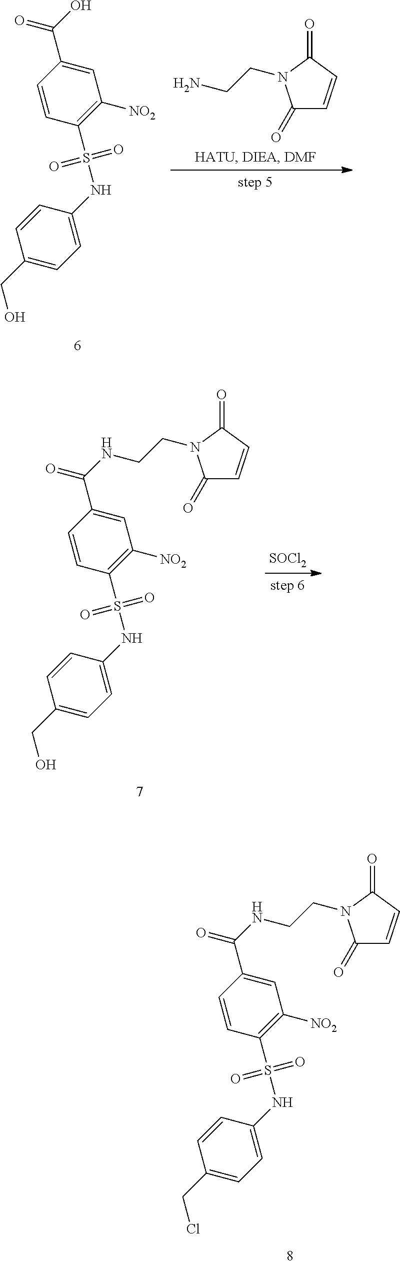 [SMILES: O=C(O)c1ccc(S(=O)(=O)Nc2ccc(CO)cc2)c([N+](=O)[O-])c1 ; NCCN1C(=O)C=CC1=O ; O=C(NCCN1C(=O)C=CC1=O)c1ccc(S(=O)(=O)Nc2ccc(CO)cc2)c([N+](=O)[O-])c1 ; "SOCl2" ; O=C(NCCN1C(=O)C=CC1=O)c1ccc(S(=O)(=O)Nc2ccc(CCl)cc2)c([N+](=O)[O-])c1]

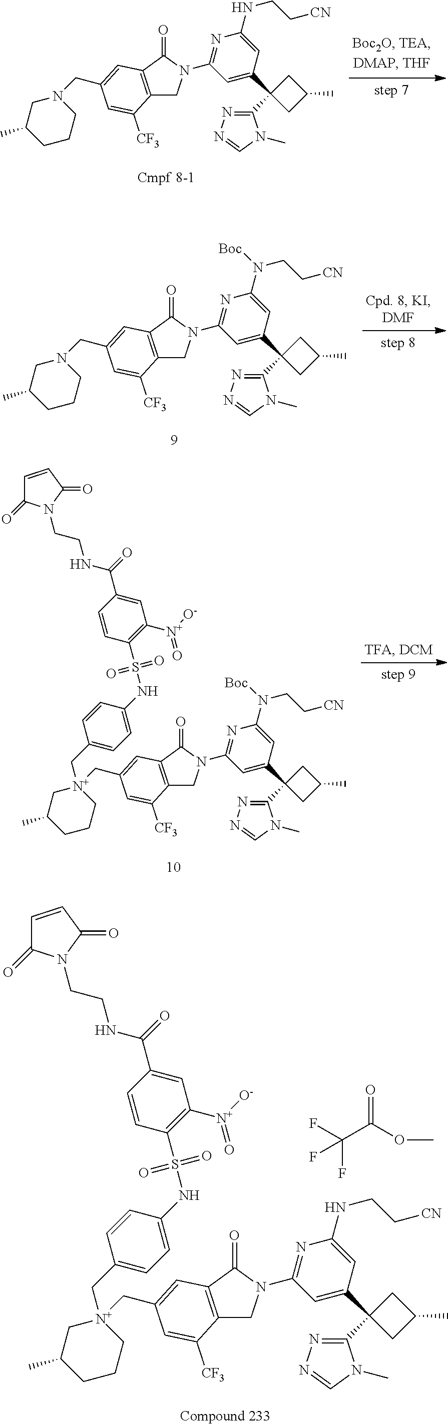

### Step 1: Synthesis of Compound 3

To a stirred mixture of methyl 4-fluoro-3-nitrobenzoate (25 g, 125.54 mmol, 1 equiv) and K₂CO₃ (34.70 g, 251.08 mmol, 2 equiv) in DMF (200 mL) was added benzyl mercaptan (31.18 g, 251.08 mmol, 2 equiv) in portions at 0° C. under nitrogen atmosphere. The resulting mixture was stirred for 3 h at room temperature under nitrogen atmosphere. LCMS indicated the reaction was completed. The reaction was quenched by the addition of Water/Ice (500 mL) at 0° C. The precipitated solids were collected by filtration and washed with PE (3×300 mL). The resulting solid was dried under infrared light. This resulted in methyl 4-(benzylsulfanyl)-3-nitrobenzoate (34 g, 89%) as a green solid. LCMS (ES, m/z): 304 [M+H]⁺

### Step 2: Synthesis of Compound 4

To a stirred solution of methyl 4-(benzylsulfanyl)-3-nitrobenzoate (25 g, 82.42 mmol, 1 equiv) in DCM (500 mL) was added HCl (aq, 6N, 500 mL) and NaClO (13%, 250 mL) in portions at 0° C. under nitrogen atmosphere. The resulting mixture was stirred for 1 h at 0° C. under nitrogen atmosphere. LCMS indicated the reaction was completed. The reaction was quenched with Water/Ice at 0° C. The resulting mixture was extracted with CH₂Cl₂ (3×500 mL). The combined organic layers were washed with brine (3×500 mL), dried over anhydrous Na₂SO₄. After filtration, the filtrate was concentrated under reduced pressure. The residue was purified by silica gel column chromatography, eluted with PE/EA (1:1) to afford methyl 4-(chlorosulfonyl)-3-nitrobenzoate (10 g, 43%) as a yellow solid. LCMS (ES, m/z): 260 [M−H-H₂O]⁻

### Step 3: Synthesis of Compound 5

To a stirred solution of β-aminobenzylalcohol (5.28 g, 42.91 mmol, 1.2 equiv) in DCM (100 mL) was added pyridine (5.66 g, 71.52 mmol, 2 equiv) and methyl 4-(chlorosulfonyl)-3-nitrobenzoate (10 g, 35.76 mmol, 1 equiv) dropwise at 0° C. under nitrogen atmosphere. The resulting mixture was stirred for 30 min at 0° C. under nitrogen atmosphere. LCMS indicated the reaction was completed. The reaction was quenched with Water/Ice at 0° C. The resulting mixture was extracted with CH₂Cl₂ (3×100 mL). The combined organic layers were washed with brine (3×100 mL), dried over anhydrous Na₂SO₄. After filtration, the filtrate was concentrated under reduced pressure. This resulted in methyl 4-{[4-(hydroxymethyl)phenyl]sulfamoyl}-3-nitrobenzoate (9 g, 68%) as a red solid. The crude product was used in the next step directly without further purification. LCMS (ES, m/z): 349 [M+H−H₂O]⁺

### Step 4: Synthesis of Compound 6

To a stirred solution of methyl 4-{[4-(hydroxymethyl)phenyl]sulfamoyl}-3-nitrobenzoate (7.5 g, 20.47 mmol, 1 equiv) in THE (750 mL) was added LiOH (0.98 g, 40.94 mmol, 2 equiv) in portions at 0° C. under nitrogen atmosphere. The resulting mixture was stirred for 1 h at 0° C. under nitrogen atmosphere. LCMS indicated the reaction was completed. The mixture was acidified to pH=3 with conc. HCl. The resulting mixture was purified by reversed-phase flash chromatography with the following conditions: column, C18 silica gel; mobile phase, ACN in Water (0.05% TFA), 5% to 60% gradient in 10 min; detector, UV 254 nm. The collected fraction was concentrated to afford 4-{[4-(hydroxymethyl)phenyl]sulfamoyl}-3-nitrobenzoic acid (3 g, 42%) as a red solid. LCMS (ES, m/z): 335 [M+H−H₂O]⁺

### Step 5: Synthesis of Compound 7

To a stirred solution of 4-{[4-(hydroxymethyl)phenyl]sulfamoyl}-3-nitrobenzoic acid (1.5 g, 4.25 mmol, 1 equiv) in DMF (15 mL) was added HATU (2.43 g, 6.38 mmol, 1.5 equiv) dropwise at 0° C. under nitrogen atmosphere. The resulting mixture was stirred for 30 min at 0° C. under nitrogen atmosphere. To the above mixture was added 1-(2-aminoethyl)pyrrole-2,5-dione (0.72 g, 5.108 mmol, 1.2 equiv) and DIEA(1.64 g, 12.75 mmol, 3 equiv) dropwise at 0° C. The resulting mixture was stirred for additional 2 h at room temperature. LCMS indicated the reaction was completed. The residue was purified by reversed-phase flash chromatography with the following conditions: column, C18 silica gel; mobile phase, ACN in Water (0.05% TFA), 5% to 60% gradient in 30 min; detector, UV 254 nm. The collected fraction was concentrated to afford N-[2-(2,5-dioxopyrrol-1-yl)ethyl]-4-{[4-(hydroxymethyl)phenyl]sulfamoyl}-3-nitrobenzamide (0.8 g, 40%) as a brown solid. LCMS (ES, m/z): 457 [M+H−H₂O]⁺

### Step 6: Synthesis of Compound 8

To a stirred mixture of N-[2-(2,5-dioxopyrrol-1-yl)ethyl]-4-{[4-(hydroxymethyl)phenyl]sulfamoyl}-3-nitrobenzamide (150 mg, 0.32 mmol, 1 equiv) and DMF (0.1 mL, 1.29 mmol, 4.09 equiv) in THE (3 mL) was added SOCl₂ (75 mg, 0.63 mmol, 2 equiv) in THE (0.05 mL) dropwise at 0° C. The resulting mixture was stirred at 0° C. for 30 min. LCMS indicated the reaction was completed. Then water (3 mL) was added into the above mixture and the resulting mixture was lyophilized to afford crude 4-{[4-(chloromethyl)phenyl]sulfamoyl}-N-[2-(2,5-dioxopyrrol-1-yl)ethyl]-3-nitrobenzamide (170 mg, crude) as a yellow solid. LCMS (ESI, m/z): 457 [M+H−HCl]⁺.

### Step 7: Synthesis of Compound 9

To a stirred mixture of 3-{[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]amino}propanenitrile (100 mg, 0.16 mmol, 1 equiv) in THE (2 mL) was added DMAP (6 mg, 0.05 mmol, 0.3 equiv) and TEA (33 mg, 0.33 mmol, 2 equiv) and Boc₂O (79 mg, 0.36 mmol, 2.2 equiv) in portions at room temperature. The resulting mixture was stirred for 3 h at 55° C. LCMS indicated the reaction was completed. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.05% TFA), 0% to 70% gradient in 35 min; detector, UV 254 nm. The collected fraction was concentrated to afford tert-butyl N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamate (85 mg, 77%) as a white solid. LCMS:(ES·m/z): 707 [M+H]⁺.

### Step 8: Synthesis of Compound 10

To a mixture of tert-butyl N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamate (130 mg, 0.18 mmol, 1 equiv) and KI (61 mg, 0.37 mmol, 2 equiv) in DMF (1.5 mL) was added 4-{[4-(chloromethyl)phenyl]sulfamoyl}-N-[2-(2,5-dioxopyrrol-1-yl)ethyl]-3-nitrobenzamide (90 mg, 0.18 mmol, 1 equiv) in DMF (0.5 mL) at 0° C. The resulting mixture was stirred for 16 h at 50° C. LCMS indicated that 31% product produced. The residue was purified by reverse flash chromatography with the following conditions: column, C18 silica gel, 80 g, 20-35 um; mobile phase, water with 0.1% TFA and ACN (0% to 100% gradient in 50 min); detector, UV 254 nm. The collected fraction was lyophilized to afford (3S)-1-[(2-{6-[(tert-butoxycarbonyl)(2-cyanoethyl)amino]-4-[(1r,3s)-3-methyl-1-(4-methyl-1,2,4-triazol-3-yl)cyclobutyl]pyridin-2-yl}-3-oxo-7-(trifluoromethyl)-1H-isoindol-5-yl)methyl]-1-{[4-(4-{[2-(2,5-dioxopyrrol-1-yl)ethyl]carbamoyl}-2-nitrobenzenesulfonamido)phenyl]methyl}-3-methylpiperidin-1-ium (68 mg, 32%) as a yellow solid. LCMS (ES, m/z): 1163 [M].

### Step 9: Synthesis of Compound 233

To a mixture of (3S)-1-[(2-{6-[(tert-butoxycarbonyl)(2-cyanoethyl)amino]-4-[(1r,3s)-3-methyl-1-(4-methyl-1,2,4-triazol-3-yl)cyclobutyl]pyridin-2-yl}-3-oxo-7-(trifluoromethyl)-1H-isoindol-5-yl)methyl]-1-{[4-(4-{[2-(2,5-dioxopyrrol-1-yl)ethyl]carbamoyl}-2-nitrobenzenesulfonamido)phenyl]methyl}-3-methylpiperidin-1-ium (60 mg, 0.052 mmol, 1 equiv) in DCM (2.5 mL) was added TFA (0.5 mL) at 25° C. The resulting mixture was stirred for 1 h at 25° C. LCMS indicated the reaction was completed. The solvent was removed under reduced pressure. The residue was purified with following conditions: Column: XSelect CSH Prep C18 OBD Column, 19*150 mm, 5 m; Mobile Phase A: Water (0.05% TFA), Mobile Phase B: ACN; Flow rate: 25 mL/min; Gradient: 20% B to 45% B in 10 min, 45% B; Wave Length: 254 nm; RT1 (min): 7.5; Number Of Runs: 0. Combined with former batches, the collected fraction was lyophilized to afford (3S)-1-[(2-{6-[(2-cyanoethyl)amino]-4-[(1r,3s)-3-methyl-1-(4-methyl-1,2,4-triazol-3-yl)cyclobutyl]pyridin-2-yl}-3-oxo-7-(trifluoromethyl)-1H-isoindol-5-yl)methyl]-1-{[4-(4-{[2-(2,5-dioxopyrrol-1-yl)ethyl]carbamoyl}-2-nitrobenzenesulfonamido)phenyl]methyl}-3-methylpiperidin-1-ium; trifluoroacetic acid (27.2 mg, 44%) as a white solid. LCMS (ESI, m/z): 1063 [M−TFA]⁺. H NMR (400 MHz, DMSO-d₆) δ 11.20 (s, 1H), 10.44 (br s, 1H), 10.01 (s, 1H), 9.02 (t, J=5.8 Hz, 1H), 8.32 (d, J=6.8 Hz, 2H), 8.22 (s, 1H), 8.16 (d, J=8.2 Hz, 1H), 8.08 (d, J=8.2, 1H), 7.65 (s, 1H), 7.41 (d, J=8.6 Hz, 2H), 7.33-7.24 (m, 3H), 7.01 (s, 2H), 6.48 (s, 1H), 5.61 (s, 2H), 5.28 (s, 2H), 4.52 (s, 2H), 3.60-3.52 (m, 4H), 3.47 (s, 3H), 3.39 (m, 4H), 3.30-3.22 (m, 1H), 2.91-2.74 (m, 5H), 2.64-2.53 (m, 3H), 1.99-1.69 (m, 4H), 1.13-1.00 (m, 4H), 0.87 (d, J=6.6 Hz, 3H).

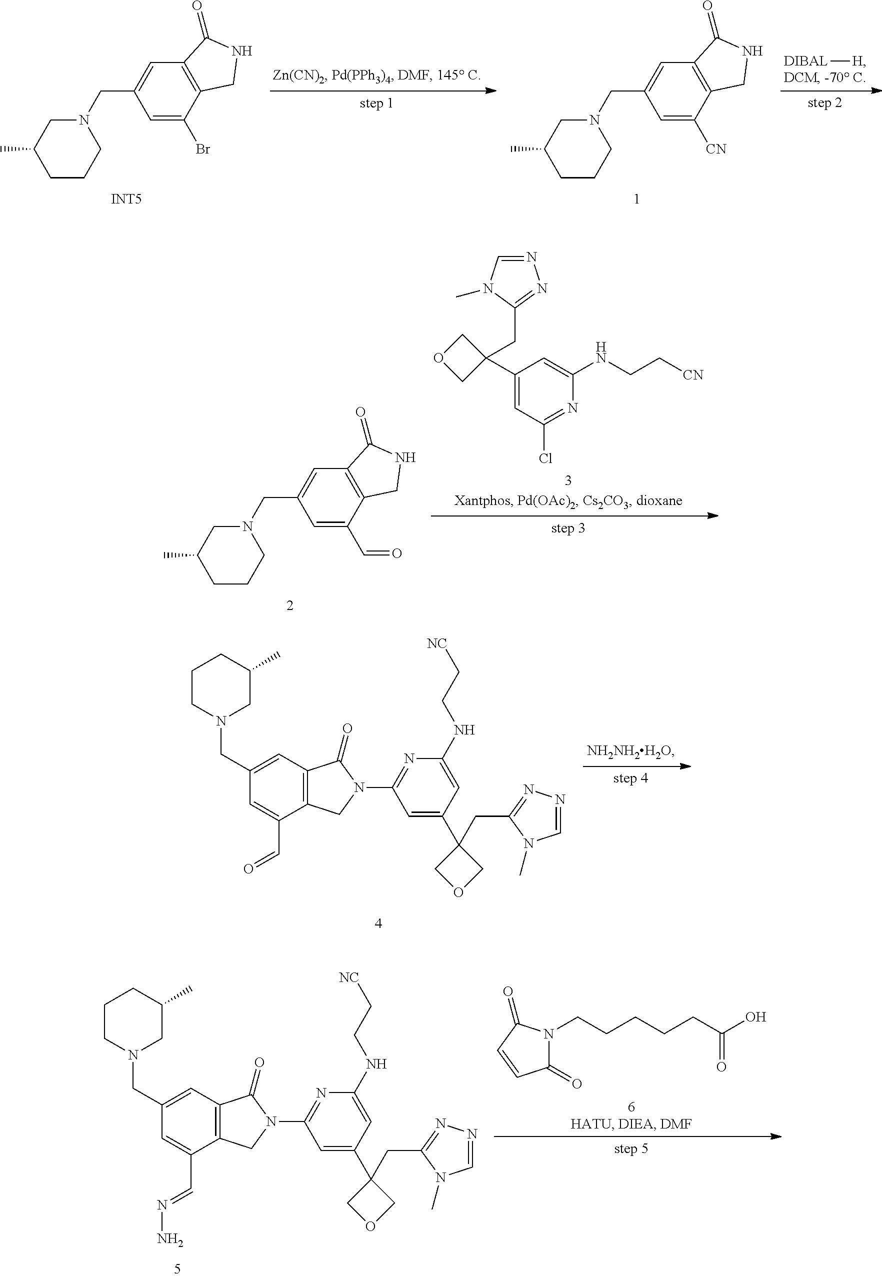

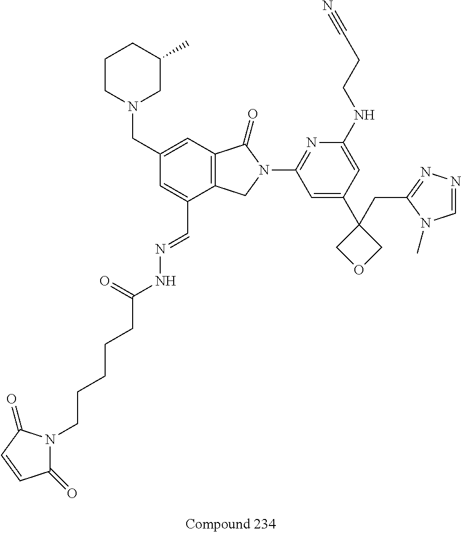 [SMILES: C[C@H]1CCCN(Cc2cc(/C=N/NC(=O)CCCCCN3C(=O)C=CC3=O)c3c(c2)C(=O)N(c2cc(C4(Cc5nncn5C)COC4)cc(NCCC#N)n2)C3)C1]

### Step 1: Synthesis of Compound 1

To a stirred solution of 4-bromo-6-{[(3S)-3-methylpiperidin-1-yl]methyl}-2,3 dihydroisoindol-1-one (1.5 g, 4.64 mmol, 1 equiv) and Zn(CN)₂ (1.09 g, 9.28 mmol, 2 equiv) in DMF (30 mL) was added Pd(PPh₃)₄ (0.54 g, 0.46 mmol, 0.1 equiv) at room temperature under nitrogen atmosphere. The resulting mixture was stirred for 2 h at 145° C. under nitrogen atmosphere. LCMS indicated the reaction was completed. The mixture was allowed to cool down to room temperature. The residue was purified by reverse flash chromatography with the following conditions: column, C18 silica gel; mobile phase, MeCN in Water (0.05% TFA), 5% to 60% gradient in 30 min; detector, UV 254 nm. The collected fraction was concentrated to afford 6-{[(3S)-3-methylpiperidin-1-yl]methyl}-1-oxo-2,3-dihydroisoindole-4-carbonitrile (400 mg, 32%) as a brown solid. LCMS (ESI, m/z):270[M+H]⁺

### Step 2: Synthesis of Compound 2

To a stirred solution of 6-{[(3S)-3-methylpiperidin-1-yl]methyl}-1-oxo-2,3-dihydroisoindole-4-carbonitrile (800 mg, 2.97 mmol, 1 equiv) in DCM (10 mL) was added DIBAl-H (9 mL, 9.0 mmol, 3.07 equiv, 1 N in DCM) dropwise at −70° C. under nitrogen atmosphere. The resulting mixture was stirred for 2 h at room temperature under nitrogen atmosphere. LCMS indicated the reaction was completed. The reaction was quenched by the addition of MeOH (5 mL) at −20° C. The resulting mixture was concentrated under reduced pressure. The residue was purified by reversed-phase flash chromatography with the following conditions: column, C18 silica gel; mobile phase, MeCN in Water (0.05% TFA), 0% to 50% gradient in 30 min; detector, UV 254 nm. The collected fraction was concentrated to afford 6-{[(3S)-3-methylpiperidin-1-yl]methyl}-1-oxo-2,3-dihydroisoindole-4-carbaldehyde (650 mg, 80%) as a yellow solid. LCMS (ESI, m/z):273[M+H]⁺

### Step 3: Synthesis of Compound 4

To a stirred mixture of 6-{[(3S)-3-methylpiperidin-1-yl]methyl}-1-oxo-2,3-dihydroisoindole-4-carbaldehyde (600 mg, 2.20 mmol, 1 equiv) and 3-[(6-chloro-4-{3-[(4-methyl-1,2,4-triazol-3-yl)methyl]oxetan-3-yl}pyridin-2-yl)amino]propanenitrile (733 mg, 2.20 mmol, 1 equiv) in dioxane (12 mL) was added Cs₂CO₃ (1435 mg, 4.40 mmol, 2 equiv), Xantphos (255 mg, 0.44 mmol, 0.2 equiv) and Pd(OAc)₂ (49 mg, 0.22 mmol, 0.1 equiv) at room temperature under air atmosphere. The resulting mixture was stirred for 1.5 h at 110° C. under nitrogen atmosphere. The mixture was allowed to cool down to room temperature. LCMS indicated the reaction was completed. The resulting mixture was concentrated under reduced pressure. The residue was purified by silica gel column chromatography, eluted with CH₂Cl₂/MeOH (10:1) to afford 3-{[6-(4-formyl-6-{[(3S)-3-methylpiperidin-1-yl]methyl}-1-oxo-3H-isoindol-2-yl)-4-{3-[(4-methyl-1,2,4-triazol-3-yl)methyl]oxetan-3-yl}pyridin-2-yl]amino}propanenitrile (250 mg, 20%) as a yellow solid. LCMS (ESI, m/z):569[M+H]⁺

### Step 4: Synthesis of Compound 5

To a stirred solution of 3-{[6-(4-formyl-6-{[(3S)-3-methylpiperidin-1-yl]methyl}-1-oxo-3H-isoindol-2-yl)-4-{3-[(4-methyl-1,2,4-triazol-3-yl)methyl]oxetan-3-yl}pyridin-2-yl]amino}propanenitrile (100 mg, 0.17 mmol, 1 equiv) in EtOH (2 mL) was added NH₂NH₂·H₂O (20 mg, 0.35 mmol, 2 equiv, 85%) at room temperature under air atmosphere. The resulting mixture was stirred for overnight at room temperature under nitrogen atmosphere. LCMS indicated the reaction was completed. The resulting mixture was concentrated under reduced pressure. The crude product was used to next step without any purification. LCMS (ESI, m/z):583[M+H]⁺

### Step 5: Synthesis of Compound 234

To a stirred solution of N-maleoyl-6-aminohexanoic acid (48 mg, 0.22 mmol, 1.2 equiv) in DMF (2 mL) was added HATU (107 mg, 0.28 mmol, 1.5 equiv) in portions at 0° C. under air atmosphere. The resulting mixture was stirred for 15 min at room temperature. To the above mixture was added 3-{[6-(4-methanehydrazonoyl-6-{[(3S)-3-methylpiperidin-1-yl]methyl}-1-oxo-3H-isoindol-2-yl)-4-{3-[(4-methyl-1,2,4-triazol-3-yl)methyl]oxetan-3-yl}pyridin-2-yl]amino}propanenitrile (110 mg, 0.19 mmol, 1 equiv) and DIEA (73 mg, 0.56 mmol, 3 equiv) in portions at 0° C. The resulting mixture was stirred for additional 1.5 h at room temperature. LCMS indicated the reaction was about 50% product. The reaction mixture was purified by Prep-HPLC with the following conditions (Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient: 22% B to 49% B in 10 min, 49% B; Wave Length: 254 nm; RT1 (min): 8.58; The collected fraction was lyophilized to afford N′-[(1E)-(2-{6-[(2-cyanoethyl)amino]-4-{3-[(4-methyl-1,2,4-triazol-3-yl)methyl]oxetan-3-yl}pyridin-2-yl}-6-{[(3S)-3-methylpiperidin-1-yl]methyl}-1-oxo-3H-isoindol-4-yl)methylidene]-6-(2,5-dioxopyrrol-1-yl)hexanehydrazide (30.5 mg, 20%) as a white solid. LCMS:(ES·m/z):776[M+H]⁺. ¹H NMR (400 MHz, DMSO-d₆) δ11.55-11.44 (m, 1H), 8.28-8.17 (m, 2H), 7.77-7.68 (m, 2H), 7.52-7.48 (m, 1H), 7.09-6.84 (m, 2H), 6.06-6.02 (m, 1H), 5.21-5.17 (m, 2H), 4.91-4.78 (m, 4H), 3.57-3.49 (m, 7H), 3.40-3.39 (m, 1H), 3.27-3.24 (m, 4H), 2.92-2.91 (m, 1H), 2.76-2.68 (m, 4H), 2.25-2.21 (m, 1H), 1.93-1.88 (m, 1H), 1.61-1.49 (m, 9H), 1.26-1.25 (m, 2H), 0.82-0.81 (m, 4H).

### Synthesis of Compound 235

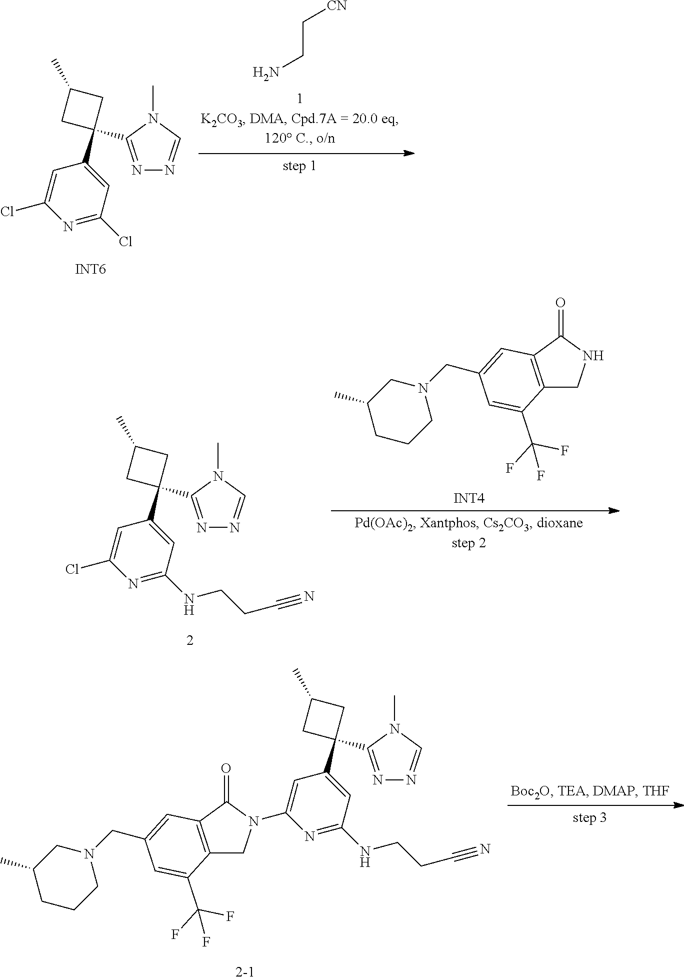

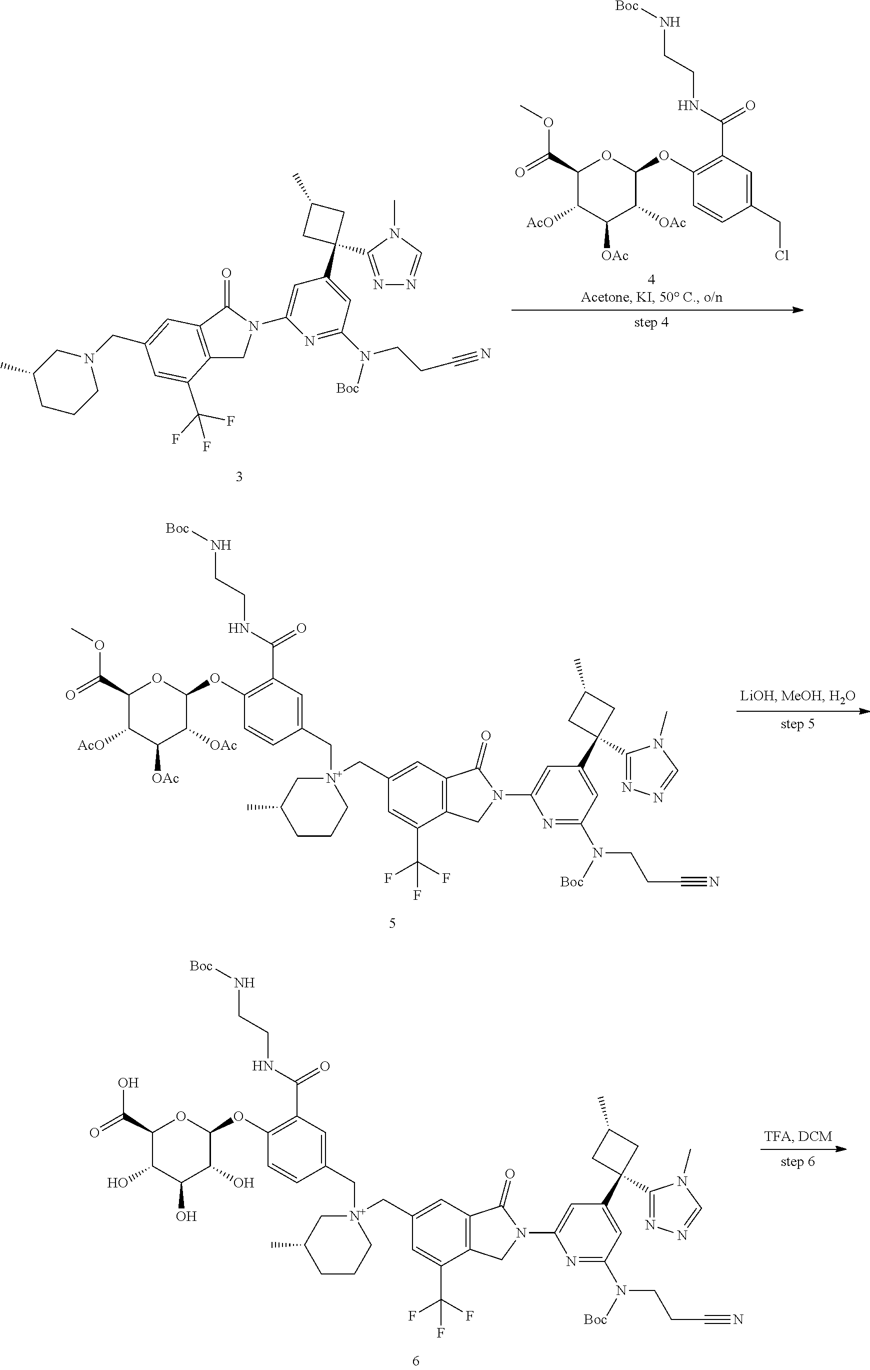

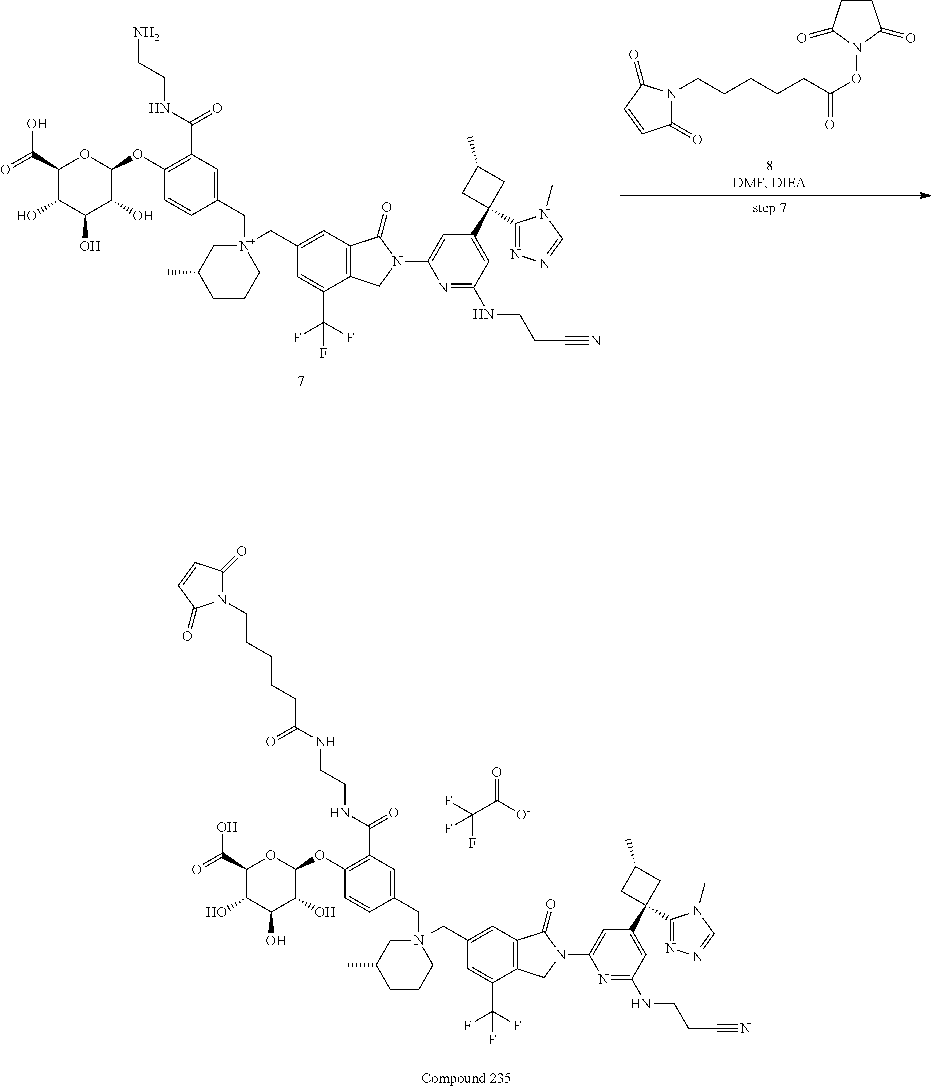 [SMILES: C[C@H]1CCC[N+](Cc2ccc(O[C@@H]3O[C@H](C(=O)O)[C@@H](O)[C@H](O)[C@H]3O)c(C(=O)NCCN)c2)(Cc2cc3c(c(C(F)(F)F)c2)CN(c2cc([C@]4(c5nncn5C)C[C@@H](C)C4)cc(NCCC#N)n2)C3=O)C1 ; O=C(CCCCCN1C(=O)C=CC1=O)ON1C(=O)CCC1=O ; C[C@H]1CCC[N+](Cc2ccc(O[C@@H]3O[C@H](C(=O)O)[C@@H](O)[C@H](O)[C@H]3O)c(C(=O)NCCNC(=O)CCCCCN3C(=O)C=CC3=O)c2)(Cc2cc3c(c(C(F)(F)F)c2)CN(c2cc([C@]4(c5nncn5C)C[C@@H](C)C4)cc(NCCC#N)n2)C3=O)C1 ; O=C([O-])C(F)(F)F]

### Step 1: Synthesis of Compound 2

To a stirred solution of 2,6-dichloro-4-[(1r,3s)-3-methyl-1-(4-methyl-1,2,4-triazol-3-yl)cyclobutyl]pyridine (5 g, 16.82 mmol, 1 equiv) in DMA (50 mL) was added K₂CO₃ (4.65 g, 33.65 mmol, 2 equiv) and β-aminopropionitrile (23.59 g, 336.50 mmol, 20 equiv) dropwise at room temperature under nitrogen atmosphere. The resulting mixture was stirred for overnight at 120° C. under nitrogen atmosphere. The mixture was allowed to cool down to room temperature. LCMS indicated the reaction was completed. The reaction mixture was purified by reversed-phase flash chromatography with the following conditions: column, C18 silica gel; mobile phase, MeCN in Water (0.05% TFA), 0% to 50% gradient in 30 min; detector, UV 254 nm. The collected fraction was concentrated to afford 3-({6-chloro-4-[(1r,3s)-3-methyl-1-(4-methyl-1,2,4-triazol-3-yl)cyclobutyl]pyridin-2-yl}amino)propanenitrile (3 g, 54%) as a yellow solid. LCMS:(ES·m/z): 331,333[M+H]⁺.

### Step 2: Synthesis of Compound 2-1

To a stirred mixture of 3-({6-chloro-4-[(1r,3s)-3-methyl-1-(4-methyl-1,2,4-triazol-3-yl)cyclobutyl]pyridin-2-yl}amino)propanenitrile (2.0 g, 6.04 mmol, 1 equiv) and 6-{[(3S)-3-methylpiperidin-1-yl]methyl}-4-(trifluoromethyl)-2,3-dihydroisoindol-1-one (2.08 g, 6.65 mmol, 1.1 equiv) in dioxane (40.00 mL) was added Cs₂CO₃ (3.94 g, 12.09 mmol, 2 equiv) and Pd(OAc)₂ (0.14 g, 0.60 mmol, 0.1 equiv) and Xantphos (0.70 g, 1.21 mmol, 0.2 equiv) in portions at room temperature under nitrogen atmosphere. The resulting mixture was stirred for 1.5 h at 100° C. under nitrogen atmosphere. The mixture was allowed to cool down to room temperature. LCMS indicated the reaction was completed. The resulting mixture was concentrated under reduced pressure. The residue was purified by silica gel column chromatography, eluted with CH₂Cl₂/MeOH (10:1) to afford 3-{[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]amino}propanenitrile (1.4 g, 38%) as a yellow solid. LCMS:(ES·m/z):607[M+H]⁺.

### Step 3: Synthesis of Compound 3

To a stirred mixture of 3-{[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]amino}propanenitrile (550 mg, 0.91 mmol, 1 equiv) in THE (12 mL) was added DMAP (33 mg, 0.27 mmol, 0.3 equiv) and TEA (183 mg, 1.81 mmol, 2 equiv) and Boc₂O (435 mg, 1.99 mmol, 2.2 equiv) in portions at room temperature under air atmosphere. The resulting mixture was stirred for 3 h at 55° C. under air atmosphere. LCMS indicated the reaction was completed. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.05% TFA), 0% to 70% gradient in 35 min; detector, UV 254 nm. The collected fraction was concentrated to afford tert-butyl N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamate (580 mg, 90%) as a white solid. LCMS:(ES·m/z):707[M+H]⁺.

### Step 4: Synthesis of Compound 5

To a stirred solution of tert-butyl N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamate (400 mg, 0.56 mmol, 1 equiv) in Acetone (8 mL) was added methyl (2S,3S,4S,5R,6S)-3,4,5-tris(acetyloxy)-6-[2-({2-[(tert-butoxycarbonyl)amino]ethyl}carbamoyl)-4-(chloromethyl)phenoxy]oxane-2-carboxylate (438 mg, 0.68 mmol, 1.2 equiv) and KI (188 mg, 1.13 mmol, 2 equiv) in portions at room temperature under air atmosphere. The resulting mixture was stirred for 2 days at 50° C. under air atmosphere. LCMS indicated the reaction was 50% product and 15% SM. The resulting mixture was concentrated under reduced pressure. The residue was purified by reversed-phase flash chromatography with the following conditions: column, C18 silica gel; mobile phase, MeCN in Water (0.05% TFA), 0% to 50% gradient in 30 min; detector, UV 254 nm. The collected fraction was concentrated to afford (3S)-1-[(2-{6-[(tert-butoxycarbonyl)(2-cyanoethyl)amino]-4-[(1r,3s)-3-methyl-1-(4-methyl-1,2,4-triazol-3-yl)cyclobutyl]pyridin-2-yl}-3-oxo-7-(trifluoromethyl)-1H-isoindol-5-yl)methyl]-1-{[3-({2-[(tert-butoxycarbonyl)amino]ethyl}carbamoyl)-4-{[(2S,3R,4S,5S,6S)-3,4,5-tris(acetyloxy)-6-(methoxycarbonyl)oxan-2-yl]oxy}phenyl]methyl}-3-methylpiperidin-1-ium (440 mg, 59%) as an off-white solid. LCMS:(ES·m/z):1316[M+H]⁺.

### Step 5: Synthesis of Compound 6

To a stirred solution of (3S)-1-[(2-{6-[(tert-butoxycarbonyl)(2-cyanoethyl)amino]-4-[(1r,3s)-3-methyl-1-(4-methyl-1,2,4-triazol-3-yl)cyclobutyl]pyridin-2-yl}-3-oxo-7-(trifluoromethyl)-1H-isoindol-5-yl)methyl]-1-{[3-({2-[(tert-butoxycarbonyl)amino]ethyl}carbamoyl)-4-{[(2S,3R,4S,5S,6S)-3,4,5-tris(acetyloxy)-6-(methoxycarbonyl)oxan-2-yl]oxy}phenyl]methyl}-3-methylpiperidin-1-ium (440 mg, 0.33 mmol, 1 equiv) in THE (200 uL) was added LiGH (32 mg, 1.34 mmol, 4 equiv) in H₂O (4 mL) in portions at 0° C. under air atmosphere. The resulting mixture was stirred for 2 h at room temperature under air atmosphere. LCMS indicated the reaction was completed. The resulting mixture was concentrated under reduced pressure. The residue was purified by reversed-phase flash chromatography with the following conditions: column, C18 silica gel; mobile phase, MeCN in Water (0.05% TFA), 5% to 70% gradient in 30 min; detector, UV 254 nm. The collected fraction was concentrated to afford (3S)-1-[(2-{6-[(tert-butoxycarbonyl)(2-cyanoethyl)amino]-4-[(1r,3s)-3-methyl-1-(4-methyl-1,2,4-triazol-3-yl)cyclobutyl]pyridin-2-yl}-3-oxo-7-(trifluoromethyl)-1H-isoindol-5-yl)methyl]-1-{[3-({2-[(tert-butoxycarbonyl)amino]ethyl}carbamoyl)-4-{[(2S,3R,4S,5S,6S)-6-carboxy-3,4,5-trihydroxyoxan-2-yl]oxy}phenyl]methyl}-3-methylpiperidin-1-ium (300 mg, 76%) as a yellow solid. LCMS:(ES·m/z):1176[M+H]⁺.

### Step 6: Synthesis of Compound 7

To a stirred solution of (3S)-1-[(2-{6-[(tert-butoxycarbonyl)(2-cyanoethyl)amino]-4-[(1r,3s)-3-methyl-1-(4-methyl-1,2,4-triazol-3-yl)cyclobutyl]pyridin-2-yl}-3-oxo-7-(trifluoromethyl)-1H-isoindol-5-yl)methyl]-1-{[3-({2-[(tert-butoxycarbonyl)amino]ethyl}carbamoyl)-4-{[(2S,3R,4S,5S,6S)-6-carboxy-3,4,5-trihydroxyoxan-2-yl]oxy}phenyl]methyl}-3-methylpiperidin-1-ium (300 mg, 0.26 mmol, 1 equiv) in DCM (6 mL) was added TFA (1.2 mL) dropwise at 0° C. under air atmosphere. The resulting mixture was stirred for 1 h at room temperature under air atmosphere. LCMS indicated the reaction was completed. The resulting mixture was concentrated under reduced pressure to afford (3S)-1-({3-[(2-aminoethyl)carbamoyl]-4-{[(2S,3R,4S,5S,6S)-6-carboxy-3,4,5-trihydroxyoxan-2-yl]oxy}phenyl}methyl)-1-[(2-{6-[(2-cyanoethyl)amino]-4-[(1r,3s)-3-methyl-1-(4-methyl-1,2,4-triazol-3-yl)cyclobutyl]pyridin-2-yl}-3-oxo-7-(trifluoromethyl)-1H-isoindol-5-yl)methyl]-3-methylpiperidin-1-ium (200 mg, 80%) as a yellow solid. The crude product was used in the next step directly without further purification. LCMS:(ES·m/z):976[M+H]⁺.

### Step 7: Synthesis of Compound 235

To a stirred mixture of (3S)-1-({3-[(2-aminoethyl)carbamoyl]-4-{[(2S,3R,4S,5S,6S)-6-carboxy-3,4,5-trihydroxyoxan-2-yl]oxy}phenyl}methyl)-1-[(2-{6-[(2-cyanoethyl)amino]-4-[(1r,3s)-3-methyl-1-(4-methyl-1,2,4-triazol-3-yl)cyclobutyl]pyridin-2-yl}-3-oxo-7-(trifluoromethyl)-1H-isoindol-5-yl)methyl]-3-methylpiperidin-1-ium (100 mg, 0.10 mmol, 1 equiv) and 2,5-dioxopyrrolidin-1-yl 6-(2,5-dioxopyrrol-1-yl)hexanoate (38 mg, 0.12 mmol, 1.2 equiv) in DMF (1 mL) was added DIEA (39 mg, 0.30 mmol, 3 equiv) dropwise at 0° C. under air atmosphere. The resulting mixture was stirred for 1.5 h at room temperature under air atmosphere. LCMS indicated the reaction was completed. The reaction mixture was purified by Prep-HPLC with the following conditions (Column: XSelect CSH Prep C18 OBD Column, 19*150 mm, 5 m; Mobile Phase A: Water (0.1% TFA), Mobile Phase B: ACN; Flow rate: 25 mL/min; Gradient: 16% B to 46% B in 7 min, 46% B; Wave Length: 254 nm; RT1 (min): 5.2; The collected fraction was lyophilized to afford (3S)-1-[(4-{[(2S,3R,4S,5S,6S)-6-carboxy-3,4,5-trihydroxyoxan-2-yl]oxy}-3-({2-[6-(2,5-dioxopyrrol-1-yl)hexanamido]ethyl}carbamoyl)phenyl)methyl]-1-[(2-{6-[(2-cyanoethyl)amino]-4-[(1r,3s)-3-methyl-1-(4-methyl-1,2,4-triazol-3-yl)cyclobutyl]pyridin-2-yl}-3-oxo-7-(trifluoromethyl)-1H-isoindol-5-yl)methyl]-3-methylpiperidin-1-ium (14.7 mg, 14%) as a white solid. LCMS:(ES·m/z):1169[M+H−TFA]⁺. ¹H NMR (400 MHz, DMSO-d₆) δ10.00 (s, 1H), 9.89 (br s, 1H), 8.31 (t, J=6.4 Hz, 1H), 8.23 (s, 2H), 7.94-7.83 (m, 3H), 7.65-7.62 (m, 1H), 7.37-7.35 (d, J=8 Hz, 1H), 7.26-7.25 (m, 1H), 7.00 (s, 2H), 6.30 (s, 1H), 5.67 (s, 2H), 5.49 (br s, 1H), 5.29 (s, 2H), 5.21-5.19 (m, 1H), 4.53 (s, 2H), 3.99-3.87 (m, 1H), 3.58-3.52 (m, 3H), 3.48 (s, 3H), 3.13-3.09 (m, 5H), 2.95-2.85 (m, 6H), 2.67-2.59 (m, 4H), 2.05 (t, J=6.4 Hz, 2H), 1.86-1.64 (m, 4H), 1.47-1.40 (m, 4H), 1.24-1.08 (m, 8H), 0.90-0.88 (m, 4H).

### Synthesis of Compound 236

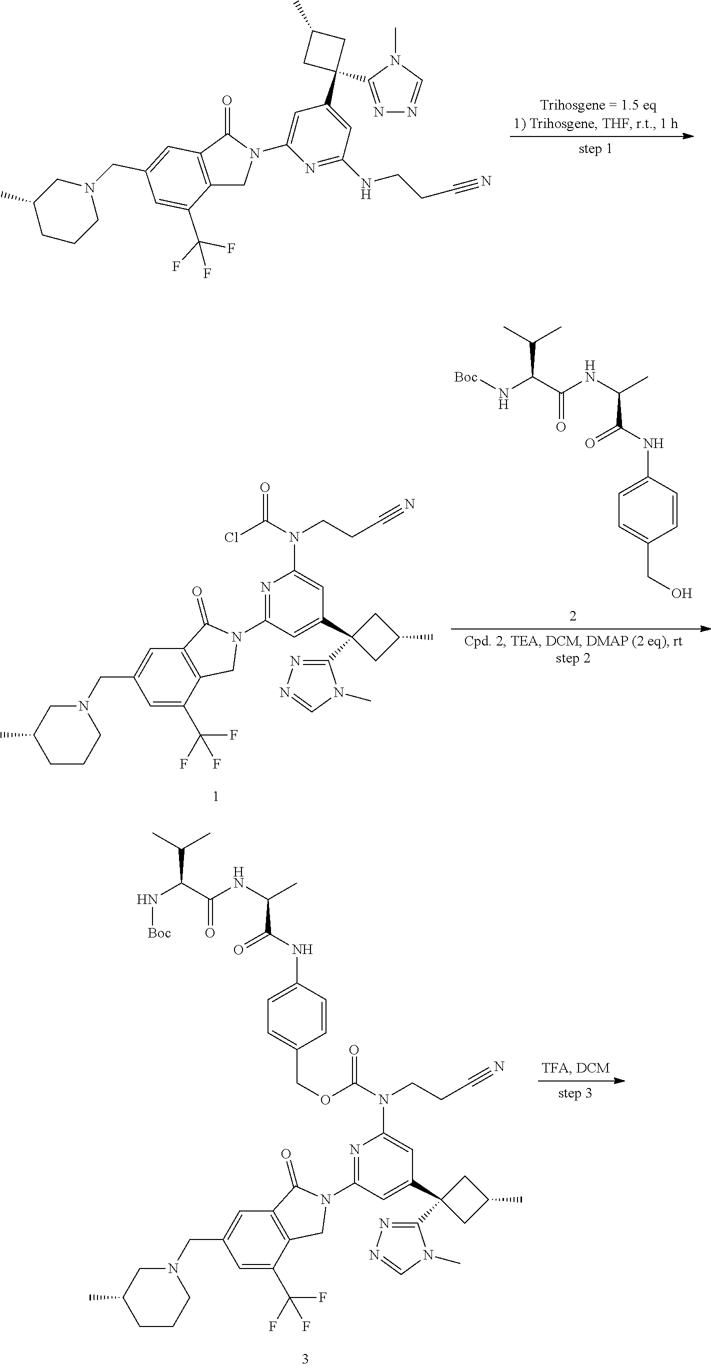

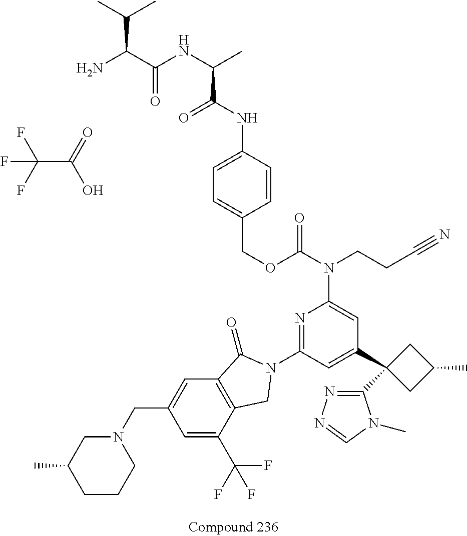

### Step 1: Synthesis of Compound 1

To a stirred solution of 3-{[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]amino}propanenitrile (400 mg, 0.66 mmol, 1 equiv) in THE (45 mL) was added Triphosgene (293 mg, 0.99 mmol, 1.5 equiv) in portions at room temperature under nitrogen atmosphere. The resulting mixture was stirred for 1 h at room temperature under nitrogen atmosphere. LCMS indicated the reaction was completed. The resulting mixture was concentrated under reduced pressure to afford N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamoyl chloride (420 mg) as a yellow solid. The crude product was used in the next step directly without further purification. LCMS:(ESI·m/z):669[M+H]⁺.

### Step 2: Synthesis of Compound 3

To a stirred mixture of tert-butyl N-[(1S)-1-{[(1S)-1-{[4-(hydroxymethyl)phenyl]carbamoyl}ethyl]carbamoyl}-2-methylpropyl]carbamate (258 mg, 0.65 mmol, 1.1 equiv) in DCM (10 mL) was added TEA (181 mg, 1.79 mmol, 3 equiv) dropwise at room temperature under air atmosphere. The resulting mixture was stirred for 30 min at room temperature under air atmosphere. To the above mixture was added N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamoyl chloride (420 mg, crude) and DMAP (146 mg, 1.19 mmol, 2 equiv) in portions at room temperature. The resulting mixture was stirred for additional overnight at room temperature. LCMS indicated the reaction was completed. The resulting mixture was concentrated under reduced pressure. The residue was purified by reverse flash chromatography with the following conditions: column, C18 silica gel; mobile phase, TFA in Water (0.05% TFA), 0% to 60% gradient in 30 min; detector, UV 254 nm. The collected fraction was concentrated to afford {4-[(2S)-2-[(2S)-2-[(tert-butoxycarbonyl)amino]-3-methylbutanamido]propanamido]phenyl}methyl N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamate (300 mg, two step 48%) as a white solid. LCMS:(ESI m/z):1026[M+H]⁺, 514[M+H]/2.

### Step 3: Synthesis of Compound 236

To a stirred solution of {4-[(2S)-2-[(2S)-2-[(tert-butoxycarbonyl)amino]-3-methylbutanamido]propanamido]phenyl}methyl N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamate (200 mg, 0.20 mmol, 1 equiv) in DCM (4 mL) was added TFA (800 uL) dropwise at 0° C. under air atmosphere. The resulting mixture was stirred for 1 h at 0° C. under air atmosphere. LCMS indicated the reaction was completed. The resulting mixture was concentrated under reduced pressure. The residue was purified by reversed-phase flash chromatography with the following conditions: column, C18 silica gel; mobile phase, MeCN in Water (0.05% TFA), 0% to 50% gradient in 30 min; detector, UV 254 nm. to afford trifluoroacetic acid; {4-[(2S)-2-[(2S)-2-amino-3-methylbutanamido]propanamido]phenyl}methyl N-(2-cyanoethyl)-N-[6-(6-{[(3S)-3-methylpiperidin-1-yl]methyl}-1-oxo-4-(trifluoromethyl)-3H-isoindol-2-yl)-4-[(1r,3s)-3-methyl-1-(4-methyl-1,2,4-triazol-3-yl)cyclobutyl]pyridin-2-yl]carbamate (127.6 mg, 57%) as an off-white solid. LCMS: (ES·m/z): 926 [M+H−TFA]⁺. ¹H NMR (400 MHz, DMSO-d₆) δ 10.22 (s, 1H), 9.83 (br s, 1H), 8.73-8.69 (m, 1H), 8.43 (s, 1H), 8.27-8.21 (m, 2H), 8.14 (s, 1H), 8.08-8.00 (m, 3H), 7.71-7.51 (m, 3H), 7.42-7.18 (m, 2H), 5.32 (s, 2H), 5.17 (s, 2H), 4.53-4.43 (m, 3H), 4.24-4.21 (m, 2H), 3.91-3.62 (m, 2H), 3.38-3.35 (m, 1H), 3.27-3.26 (m, 1H), 3.18 (s, 3H), 3.01-2.99 (m, 2H), 2.97-2.73 (m, 1H), 2.70-2.68 (m, 2H), 2.67-2.57 (m, 3H), 2.44-2.40 (m, 1H), 2.08-2.05 (m, 1H), 1.85-1.72 (m, 4H), 1.36 (d, J=8 Hz, 3H), 1.05 (d, J=4 Hz, 3H), 0.96-0.93 (m, 6H), 0.88 (d, J=4 Hz, 2H).

### Synthesis of Compound 237

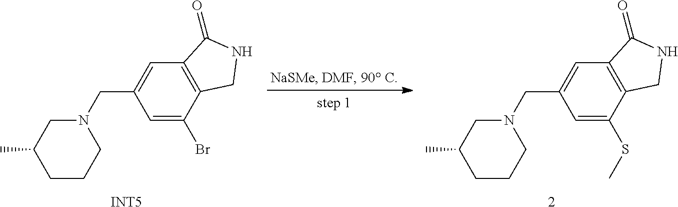

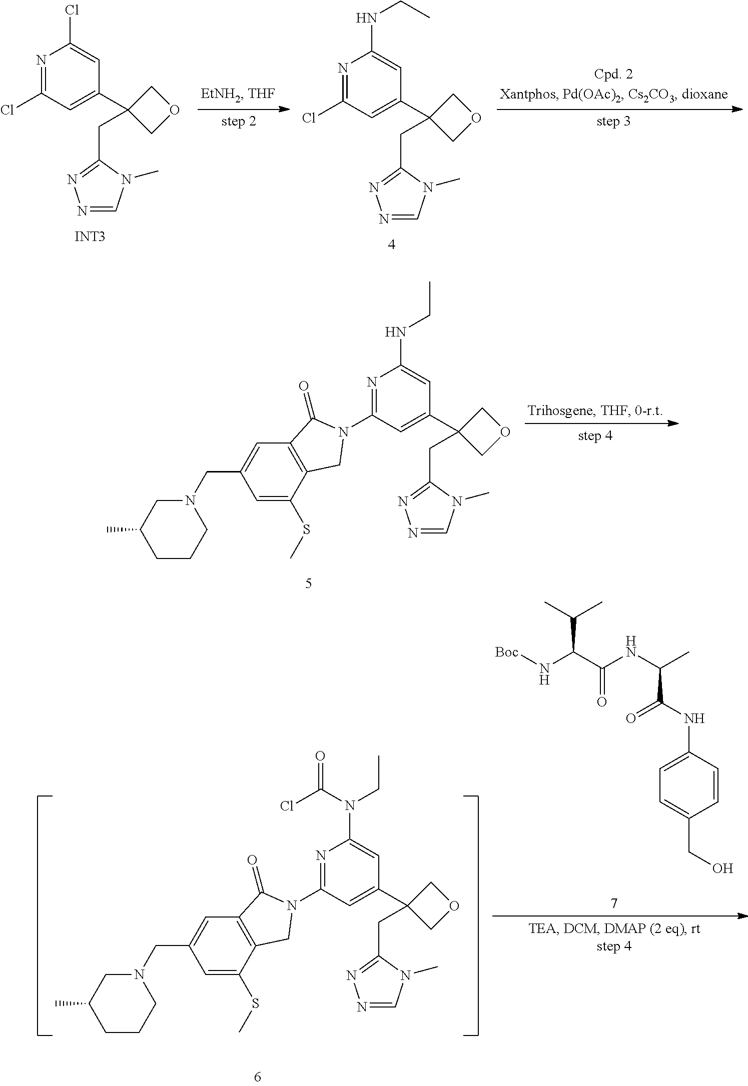

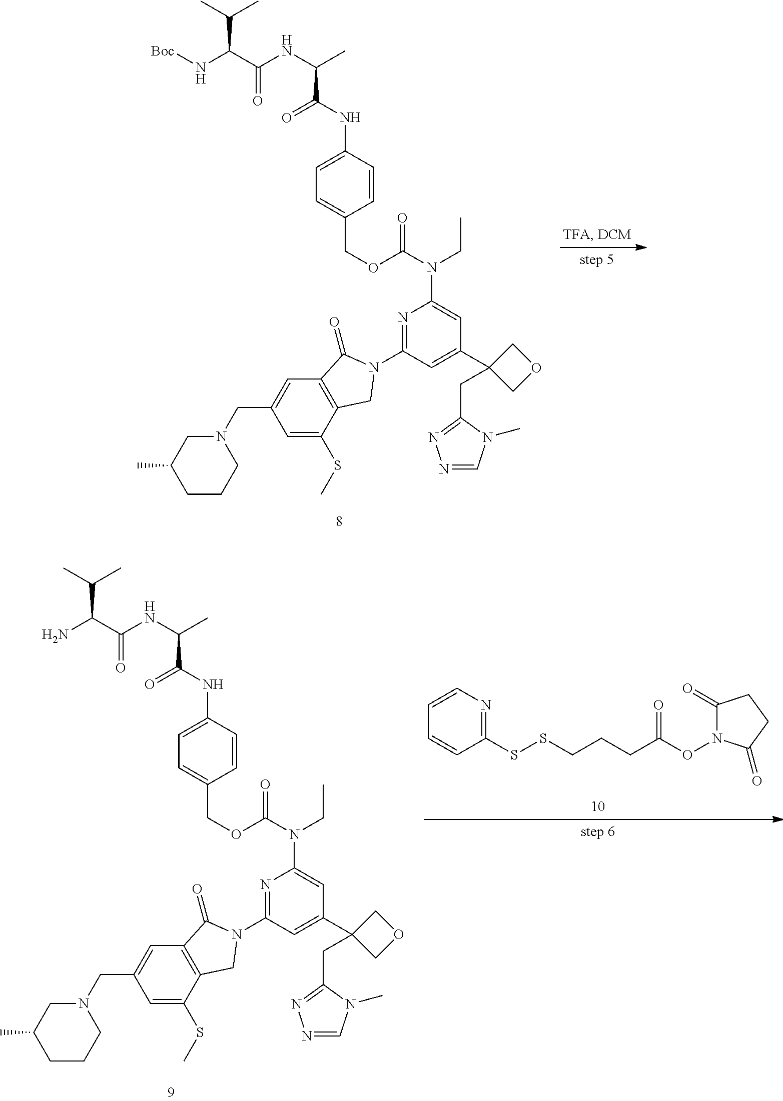 [SMILES: CCN(C(=O)OCc1ccc(NC(=O)[C@H](C)NC(=O)[C@@H](NC(=O)OC(C)(C)C)C(C)C)cc1)c1cc(C2(Cc3nncn3C)COC2)cc(N2Cc3c(SC)cc(CN4CCC[C@H](C)C4)cc3C2=O)n1 ; CCN(C(=O)OCc1ccc(NC(=O)[C@H](C)NC(=O)[C@@H](N)C(C)C)cc1)c1cc(C2(Cc3nncn3C)COC2)cc(N2Cc3c(SC)cc(CN4CCC[C@H](C)C4)cc3C2=O)n1 ; O=C(CCCSSc1ccccn1)ON1C(=O)CCC1=O]

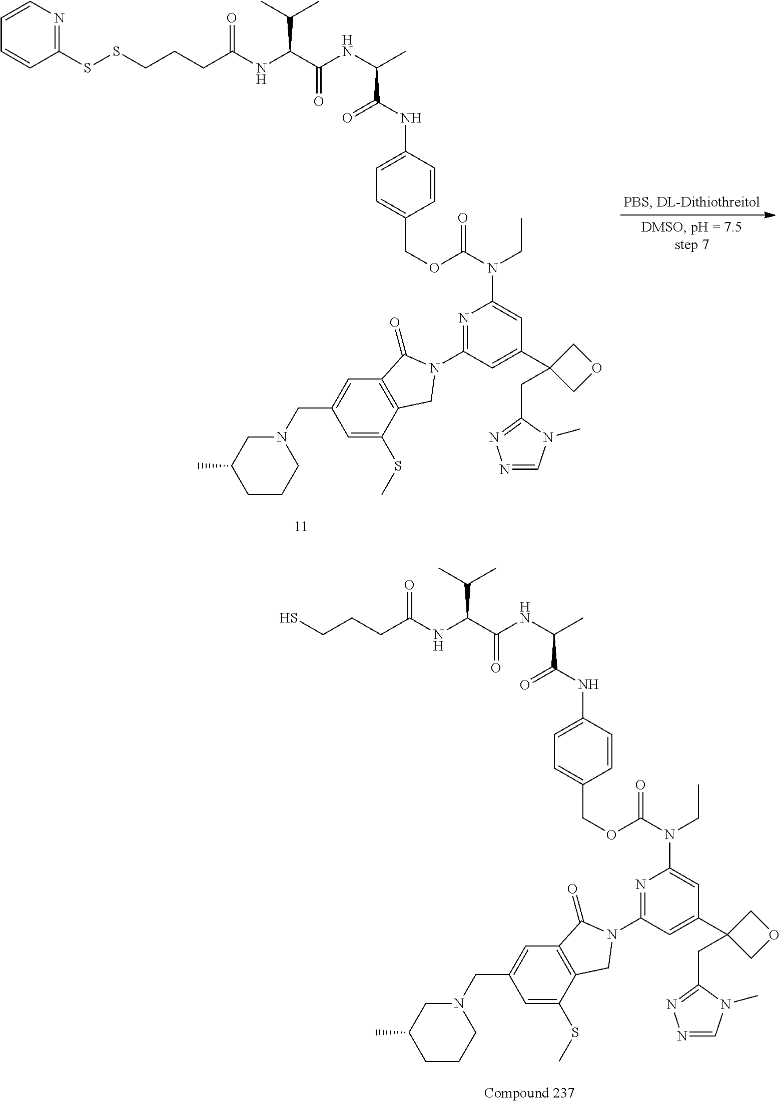 [SMILES: CCN(C(=O)OCc1ccc(NC(=O)[C@H](C)NC(=O)[C@@H](NC(=O)CCCSSc2ccccn2)C(C)C)cc1)c1cc(C2(Cc3nncn3C)COC2)cc(N2Cc3c(SC)cc(CN4CCC[C@H](C)C4)cc3C2=O)n1 ; CCN(C(=O)OCc1ccc(NC(=O)[C@H](C)NC(=O)[C@@H](NC(=O)CCCS)C(C)C)cc1)c1cc(C2(Cc3nncn3C)COC2)cc(N2Cc3c(SC)cc(CN4CCC[C@H](C)C4)cc3C2=O)n1]

### Step 1: Synthesis of Compound 2

To a stirred solution of 4-bromo-6-{[(3S)-3-methylpiperidin-1-yl]methyl}-2,3-dihydroisoindol-1-one (200 mg, 0.62 mmol, 1 equiv) in DMF (3 mL) was added (methylsulfanyl)sodium (93 mg, 0.93 mmol, 1.5 equiv) at 25° C. The resulting mixture was stirred for 1 h at 90° C. LCMS indicated the reaction was completed. The reaction was purified by reverse flash chromatography with the following conditions: column, C18 silica gel; mobile phase, TFA in water, 0% to 50% gradient in 40 min; detector, UV 254 nm. This resulted in 6-{[(3S)-3-methylpiperidin-1-yl]methyl}-4-(methylsulfanyl)-2,3-dihydroisoindol-1-one (80 mg, 44%) as a white solid. LCMS:(ES·m/z): 291 [M+H]⁺.

### Step 2: Synthesis of Compound 4

To a stirred solution of 2,6-dichloro-4-{3-[(4-methyl-1,2,4-triazol-3-yl)methyl]oxetan-3-yl}pyridine (1.3 g, 4.34 mmol, 1 equiv) and in ethylamine solution (2.0 M in THF, 13 mL) was stirred for overnight at 90° C. LCMS indicated the reaction was completed. The mixture was allowed to cool down to room temperature. The resulting mixture was concentrated under vacuum. The residue was purified by silica gel column chromatography, eluted with CH₂Cl₂/MeOH (10:1) to afford 6-chloro-N-ethyl-4-{3-[(4-methyl-1,2,4-triazol-3-yl)methyl]oxetan-3-yl}pyridin-2-amine (440 mg, 33%) as a yellow solid. LCMS (ESI, ms): 308,310 [M+H]⁺.

### Step 3: Synthesis of Compound 5

To a stirred mixture of 6-{[(3S)-3-methylpiperidin-1-yl]methyl}-4-(methylsulfanyl)-2,3-dihydroisoindol-1-one (238 mg, 0.82 mmol, 1.0 equiv) and 6-chloro-N-ethyl-4-{3-[(4-methyl-1,2,4-triazol-3-yl)methyl]oxetan-3-yl}pyridin-2-amine (250 mg, 0.82 mmol, 1.0 equiv) in dioxane (4 mL) was added Xantphos (94 mg, 0.16 mmol, 0.2 equiv), Cs₂CO₃ (530 mg, 1.64 mmol, 2 equiv) and Pd(OAc)₂ (16 mg, 0.082 mmol, 0.1 equiv) at 25° C. The resulting mixture was stirred for 2 h at 120° C. under nitrogen atmosphere. LCMS indicated the reaction was completed. The solvent was removed under reduced pressure. The residue was purified by reverse flash chromatography with the following conditions: column, C18 silica gel, 80 g, 20-35 um; mobile phase, water with 0.1% TFA and ACN (5% to 95% gradient in 50 min); detector, UV 254 nm. The eluent was lyophilized to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (230 mg, 50%) as a yellow solid. LCMS (ESI, m/z): 562 [M+H]⁺.

### Step 4: Synthesis of Compound 8

To a stirred solution of 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (900 mg, 1.60 mmol, 1 equiv) in THF (80 mL) were added Triphosgene (713 mg, 2.40 mmol, 1.5 equiv) in portions at room temperature under nitrogen atmosphere. The resulting mixture was stirred for additional 30 min at room temperature. The resulting mixture was concentrated under vacuum. To the above mixture was added tert-butyl N-[(1S)-1-{[(1S)-1-{[4-(hydroxymethyl)phenyl]carbamoyl}ethyl]carbamoyl}-2-methylpropyl]carbamate (630 mg, 1.60 mmol, 1 equiv) in DCM (20 mL) were added TEA (486.37 mg, 4.806 mmol, 3 equiv) at 0° C. The resulting mixture was stirred for additional 10 min at 0° C. Finally add DMAP (391 mg, 3.20 mmol, 2 equiv) in portion. The resulting mixture was stirred for overnight at room temperature under nitrogen atmosphere. ˜10% desire product could be detected by LCMS. The resulting mixture was concentrated under reduced pressure. The residue was purified by reverse flash chromatography with the following conditions: column, C18 silica gel; mobile phase, MeCN in Water (0.1% TFA), 10% to 50% gradient in 10 min; detector, UV 254 nm. This resulted in {4-[(2S)-2-[(2S)-2-[(tert-butoxycarbonyl)amino]-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (180 mg, 10%) as a white solid. LCMS: (ES, m/s): 981 [M+H]⁺.

### Step 5: Synthesis of Compound 9

A mixture of {4-[(2S)-2-[(2S)-2-[(tert-butoxycarbonyl)amino]-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (180 mg, 0.18 mmol, 1 equiv) in DCM (7.5 mL) was added TFA (1.5 mL) at 25° C. The resulting mixture was stirred for 1 h at 25° C. LCMS indicated the reaction was completed. The solvent was removed under reduced pressure. The residue was lyophilized to afford crude {4-[(2S)-2-[(2S)-2-amino-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (180 mg, crude) as a brown solid. LCMS (ESI, m/z): 881 [M+H]⁺

### Step 6: Synthesis of Compound 11

To a stirred solution of {4-[(2S)-2-[(2S)-2-amino-3-methylbutanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (90 mg, 0.10 mmol, 1.0 equiv) and DIEA (26 mg, 0.20 mmol, 2.0 equiv) in DMF (1 mL) was added 2,5-dioxopyrrolidin-1-yl 4-(pyridin-2-yldisulfanyl)butanoate (40 mg, 0.12 mmol, 1.2 equiv) at room temperature under nitrogen atmosphere. The resulting mixture was stirred for 1 h at room temperature under nitrogen atmosphere. The reaction was monitored by LCMS. The reaction mixture was dried by lyophilization to afford {4-[(2S)-2-[(2S)-3-methyl-2-[4-(pyridin-2-yldisulfanyl)butanamido]butanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (90 mg, 80%) as a white solid. The crude product was used in the next step directly without further purification. LCMS (ES, m/z) =1092 [M+H]⁺

### Step 7: Synthesis of Compound 237

To a stirred solution of {4-[(2S)-2-[(2S)-3-methyl-2-[4-(pyridin-2-yldisulfanyl)butanamido]butanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (90 mg, 0.082 mmol, 1.0 equiv) and DTT (63 mg, 0.41 mmol, 5.0 equiv) in DMSO (1.5 mL) and PBS (pH=7.4, 0.1N, 0.5 mL) at room temperature under air atmosphere. The reaction was monitored by LCMS. Desired product could be detected by LCMS. The crude product (90 mg) was purified by Prep-HPLC with the following conditions (Column: XBridge Prep OBD C18 Column, 19*250 mm, 5 m; Mobile Phase A: Water (0.1% FA), Mobile Phase B: ACN; Flow rate: 25 mL/min mL/min; Gradient: 17% B to 47% B in 15 min; Wave Length: 254 nm/220 nm; RT1 (min): 14.1) and the solvent was removed by lyophilization to afford {4-[(2S)-2-[(2S)-3-methyl-2-(4-sulfanylbutanamido)butanamido]propanamido]phenyl}methyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (17.4 mg, 20.06%) as a white solid. LCMS (ES, m/z)=984 [M+H]⁺. ¹H NMR (400 MHz, DMSO-d₆) δ 10.00 (s, 1H), 8.22-8.20 (m, 2H), 7.97 (d, J=1.2 Hz, 1H), 7.89 (d, J=8.4 Hz, 1H), 7.61 (d, J=8.4 Hz, 2H), 7.48 (t, J=4.4 Hz, 2H), 7.39-7.34 (m, 2H), 7.19 (s, 1H), 5.13 (s, 2H), 4.94-4.83 (m, 4H), 4.76 (d, J=6.0 Hz, 2H), 4.40-4.36 (m, 1H), 4.21-4.14 (m, 1H), 3.93-3.91 (m, 2H), 3.55 (s, 2H), 3.49 (s, 2H), 3.18 (s, 3H), 2.69-2.67 (m, 2H), 2.58 (s, 3H), 2.47-2.40 (m, 2H), 2.34-2.22 (m, 3H), 2.00-1.70 (m, 4H), 1.68-1.55 (m, 4H), 1.49-1.45 (m, 1H), 1.30 (d, J=7.2 Hz, 3H), 1.21 (t, J=7.2 Hz, 3H), 0.90-0.76 (m, 10H).

### Synthesis of Compound 238

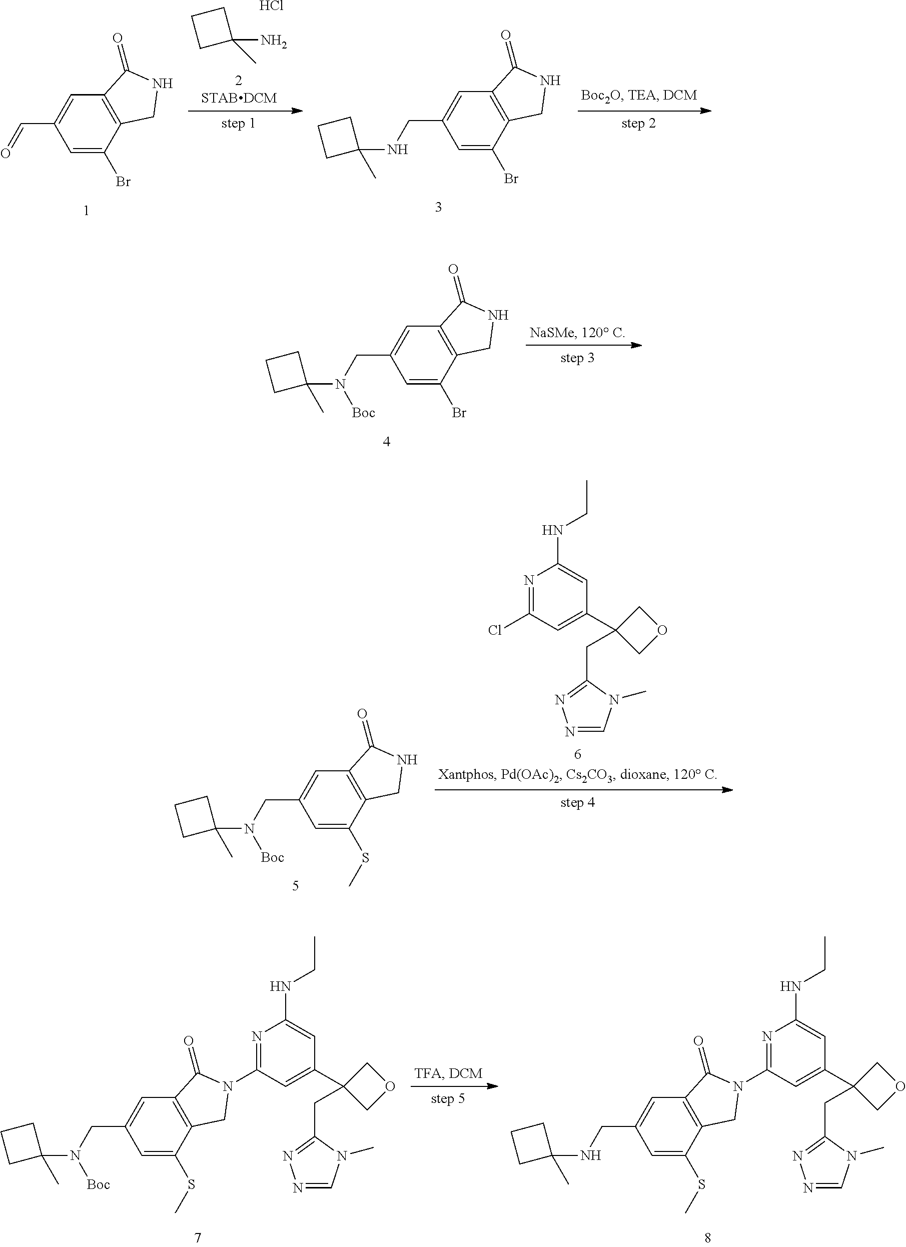

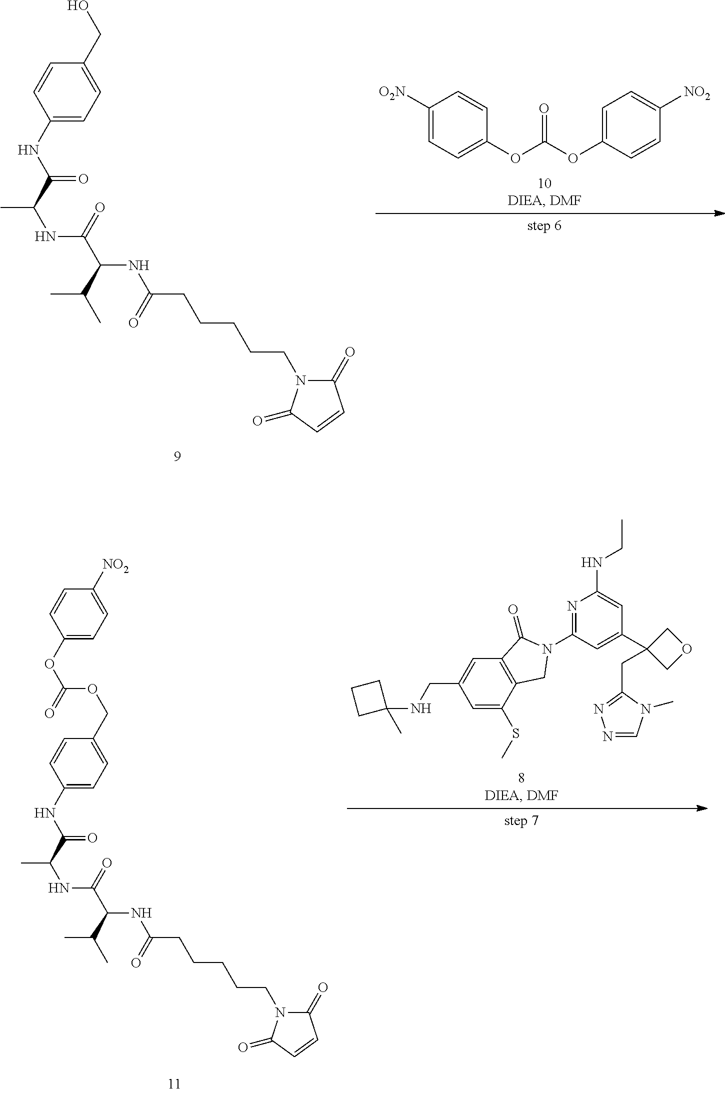 [SMILES: CC(C)[C@H](NC(=O)CCCCCN1C(=O)C=CC1=O)C(=O)N[C@@H](C)C(=O)Nc1ccc(CO)cc1 ; O=C(Oc1ccc([N+](=O)[O-])cc1)Oc1ccc([N+](=O)[O-])cc1 ; CC(C)[C@H](NC(=O)CCCCCN1C(=O)C=CC1=O)C(=O)N[C@@H](C)C(=O)Nc1ccc(COC(=O)Oc2ccc([N+](=O)[O-])cc2)cc1 ; CCNc1cc(C2(Cc3nncn3C)COC2)cc(N2Cc3c(SC)cc(CNC4(C)CCC4)cc3C2=O)n1]

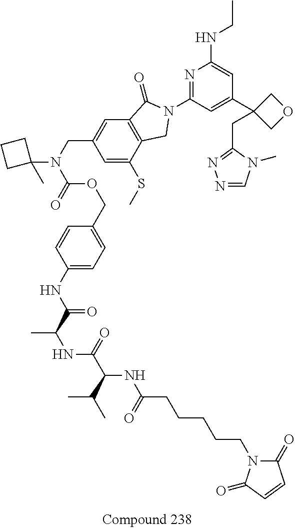 [SMILES: CCNc1cc(C2(Cc3nncn3C)COC2)cc(N2Cc3c(SC)cc(CN(C(=O)OCc4ccc(NC(=O)[C@H](C)NC(=O)[C@@H](NC(=O)CCCCCN5C(=O)C=CC5=O)C(C)C)cc4)C4(C)CCC4)cc3C2=O)n1]

### Step 1: Synthesis of Compound 3

A solution of 7-bromo-3-oxo-1,2-dihydroisoindole-5-carbaldehyde (3 g, 12.50 mmol, 1.0 equiv) in DCM (30 mL) was treated with 1-methylcyclobutan-1-amine hydrochloride (2.28 g, 18.75 mmol, 1.5 equiv) and TEA (3.79 g, 37.50 mmol, 3.0 equiv). The reaction mixture was stirred for 30 min at room temperature. To the above mixture was added STAB (7.95 g, 37.50 mmol, 3.0 equiv) in portions. The resulting mixture was stirred for additional overnight at room temperature. Desired product could be detected by LCMS. The resulting mixture was concentrated under reduced pressure. The residue was purified by silica gel column chromatography, eluted with CH₂Cl₂/MeOH (10:1) to afford 4-bromo-6-{[(1-methylcyclobutyl)amino]methyl}-2,3-dihydroisoindol-1-one (3.6 g, 74%) as a white solid. LCMS (ES, m/z): 309,311 [M+H]⁺.

### Step 2: Synthesis of Compound 4

A solution of 4-bromo-6-{[(1-methylcyclobutyl)amino]methyl}-2,3-dihydroisoindol-1-one (3.60 g, 11.64 mmol, 1.0 equiv) in THE (36 mL) was basified to pH=9 with NaHCO₃ (sat.aq) and the mixture was treated with di-tert-butyl dicarbonate (3.05 g, 13.97 mmol, 1.2 equiv). The resulting mixture was stirred for 16 h at room temperature. Desired product could be detected by LCMS. The resulting mixture was concentrated under reduced pressure. The residue was purified by silica gel column chromatography, eluted with CH₂Cl₂/MeOH (10:1) to afford tert-butyl N-[(7-bromo-3-oxo-1,2-dihydroisoindol-5-yl)methyl]-N-(1-methylcyclobutyl)carbamate (2.70 g, 49%) as a white solid. LCMS (ES, m/z): 409,411 [M+H]⁺.

### Step 3: Synthesis of Compound 5

A solution of tert-butyl N-[(7-bromo-3-oxo-1,2-dihydroisoindol-5-yl)methyl]-N-(1-methylcyclobutyl)carbamate (2.0 g, 4.89 mmol, 1.0 equiv) in DMF (10 mL) was treated with NaSMe (0.68 g, 9.77 mmol, 2.0 equiv). The resulting mixture was stirred for 1 h at 120° C. under nitrogen atmosphere. Desired product could be detected by LCMS. The reaction mixture was allowed to cool down to room temperature and quenched with water at room temperature. The resulting mixture was extracted with EtOAc (3×10 mL). The combined organic layers were washed with water (10 mL), dried over anhydrous Na₂SO₄. After filtration, the filtrate was concentrated under reduced pressure. The residue was purified by silica gel column chromatography, eluted with CH₂Cl₂/MeOH (10:1) to afford tert-butyl N-(1-methylcyclobutyl)-N-{[7-(methylsulfanyl)-3-oxo-1,2-dihydroisoindol-5-yl]methyl}carbamate (1.90 g, 87%) as an off-white solid. LCMS (ES, m/z): 377 [M+H]⁺.

### Step 4: Synthesis of Compound 7

A solution of tert-butyl N-(1-methylcyclobutyl)-N-{[7-(methylsulfanyl)-3-oxo-1,2-dihydroisoindol-5-yl]methyl}carbamate (400 mg, 1.06 mmol, 1.0 equiv) in dioxane (5 mL) was treated with 6-chloro-N-ethyl-4-{3-[(4-methyl-1,2,4-triazol-3-yl)methyl]oxetan-3-yl}pyridin-2-amine (327 mg, 1.06 mmol, 1.0 equiv), Pd(OAc)₂ (48 mg, 0.21 mmol, 0.2 equiv), [5-(diphenylphosphanyl)-9,9-dimethyl-9H-xanthen-4-yl]diphenylphosphane (246 mg, 0.43 mmol, 0.4 equiv) and Cs₂CO₃ (692 mg, 2.12 mmol, 2.0 equiv) under nitrogen atmosphere. The resulting mixture was stirred for 1.5 h at 120° C. under nitrogen atmosphere. Desired product could be detected by LCMS. The reaction mixture was allowed to cool down to room temperature and concentrated to dryness under vacuum. The residue was purified by silica gel column chromatography, eluted with CH₂Cl₂/MeOH (10:1) to afford tert-butyl N-({2-[6-(ethylamino)-4-{3-[(4-methyl-1,2,4-triazol-3-yl)methyl]oxetan-3-yl}pyridin-2-yl]-7-(methylsulfanyl)-3-oxo-1H-isoindol-5-yl}methyl)-N-(1-methylcyclobutyl)carbamate (190 mg, 22%) as an off-white solid. LCMS (ES, m/z): 648 [M+H]⁺.

### Step 5: Synthesis of Compound 8

A solution of tert-butyl N-({2-[6-(ethylamino)-4-{3-[(4-methyl-1,2,4-triazol-3-yl)methyl]oxetan-3-yl}pyridin-2-yl]-7-(methylsulfanyl)-3-oxo-1H-isoindol-5-yl}methyl)-N-(1-methylcyclobutyl)carbamate (90 mg, 0.14 mmol, 1.0 equiv) in DCM (3 mL) was treated with TFA (1 mL). The resulting mixture was stirred for 1 h at room temperature. Desired product could be detected by LCMS. The reaction mixture was concentrated to dryness under vacuum. The residue was submitted to Prep-HPLC purification (Column: Xselect CSH Prep C18 Column, 30*150 mm, 5 m; Mobile Phase A: Water (0.05% TFA), Mobile Phase B: ACN; Flow rate: 60 mL/min mL/min; Gradient: 10% B to 40% B in 10 min; Wave Length: 254 nm/220 nm; RT1 (min): 8.68) and the collected fractions were lyophilized to afford 2-[6-(ethylamino)-4-{3-[(4-methyl-1,2,4-triazol-3-yl)methyl]oxetan-3-yl}pyridin-2-yl]-6-{[(1-methylcyclobutyl)amino]methyl}-4-(methylsulfanyl)-3H-isoindol-1-one; trifluoroacetic acid (25.50 mg, 27%) as a yellow solid. LCMS (ESI, m/z): 548 [M+H]⁺.

### Step 6: Synthesis of Compound 11

A solution of 6-(2,5-dioxopyrrol-1-yl)-N-[(1S)-1-{[(1S)-1-{[4-(hydroxymethyl)phenyl]carbamoyl}ethyl]carbamoyl}-2-methylpropyl]hexanamide (400 mg, 0.82 mmol, 1.0 equiv) and DIEA (212 mg, 1.64 mmol, 2.0 equiv) in DMF (2 mL) was treated with bis(4-nitrophenyl) carbonate (300 mg, 0.99 mmol, 1.2 equiv) room temperature. The resulting mixture was stirred for 1 h at room temperature. Desired product could be detected by LCMS. The resulting mixture was concentrated under reduced pressure. The residue was purified by reverse flash chromatography: water with 0.05% TFA and ACN (0% to 50% gradient in 50 min); detector, UV 254 nm. This resulted in {4-[(2S)-2-[(2S)-2-[6-(2,5-dioxopyrrol-1-yl)hexanamido]-3-methylbutanamido]propanamido]phenyl}methyl 4-nitrophenyl carbonate (350 mg, 63%) as an off-white solid. LCMS (ES, m/z): 652 [M+H]⁺.

### Step 7: Synthesis of Compound 238

A solution of {4-[(2S)-2-[(2S)-2-[6-(2,5-dioxopyrrol-1-yl)hexanamido]-3-methylbutanamido]propanamido]phenyl}methyl 4-nitrophenyl carbonate (78 mg, 0.12 mmol, 1.1 equiv) in DMF (0.5 mL) was treated with 2-[6-(ethylamino)-4-{3-[(4-methyl-1,2,4-triazol-3-yl)methyl]oxetan-3-yl}pyridin-2-yl]-6-{[(1-methylcyclobutyl)amino]methyl}-4-(methylsulfanyl)-3H-isoindol-1-one (60 mg, 0.11 mmol, 1.0 equiv). The resulting mixture was stirred for overnight at 50° C. Desired product could be detected by LCMS. The reaction mixture was submitted to Prep-HPLC purification (Column: XBridge Prep OBD C18 Column, 30*150 mm, 5 m; Mobile Phase A: Water (0.05% TFA), Mobile Phase B: ACN; Flow rate: 60 mL/min mL/min; Gradient: 27% B to 57% B in 10 min; Wave Length: 254 nm/220 nm; RT1 (min): 8.83) and the collected fractions were lyophilized to afford trifluoroacetic acid; {4-[(2S)-2-[(2S)-2-[6-(2,5-dioxopyrrol-1-yl)hexanamido]-3-methylbutanamido]propanamido]phenyl}methyl N-({2-[6-(ethylamino)-4-{3-[(4-methyl-1,2,4-triazol-3-yl)methyl]oxetan-3-yl}pyridin-2-yl]-7-(methylsulfanyl)-3-oxo-1H-isoindol-5-yl}methyl)-N-(1-methylcyclobutyl)carbamate (10.30 mg, 7.97%) as a white solid. LCMS (ESI, m/z): 1061 [M+H−TFA]⁺. ¹H NMR (400 MHz, DMSO-d₆) δ 9.96 (s, 1H), 9.10 (s, 1H), 8.16 (d, J=6.8 Hz, 1H), 7.81 (d, J=8.4 Hz, 1H), 7.59 (br s, 2H), 7.42 (d, J=5.2 Hz, 2H), 7.29 (s, 2H), 7.00 (s, 2H), 6.00 (s, 1H), 5.03 (br s, 2H), 4.92-4.78 (m, 6H), 4.42-4.39 (m, 3H), 4.18-4.16 (m, 1H), 3.68 (s, 2H), 3.51 (s, 1H), 3.41-3.38 (m, 3H), 3.36 (d, J=7.2 Hz, 2H), 3.26-3.24 (m, 2H), 2.52-2.51 (m, 2H), 2.49-2.48 (m, 2H), 2.23-2.05 (m, 4H), 2.01-1.83 (m, 3H), 1.75-1.61 (m, 2H), 1.55-1.48 (m, 4H), 1.41-1.28 (m, 6H), 1.23-1.17 (m, 5H), 0.85-0.82 (m, 6H).

### Synthesis of Compound 239

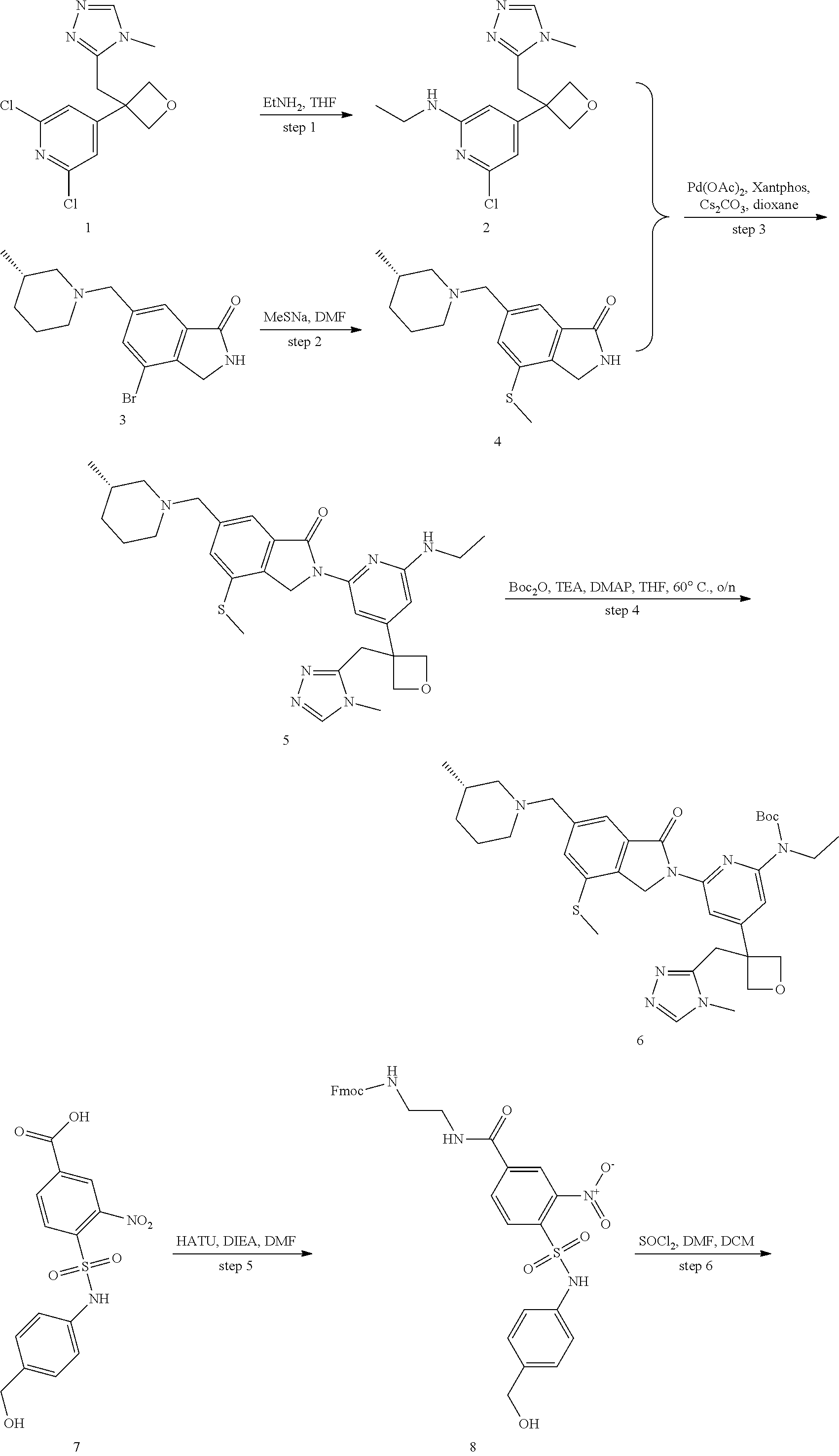

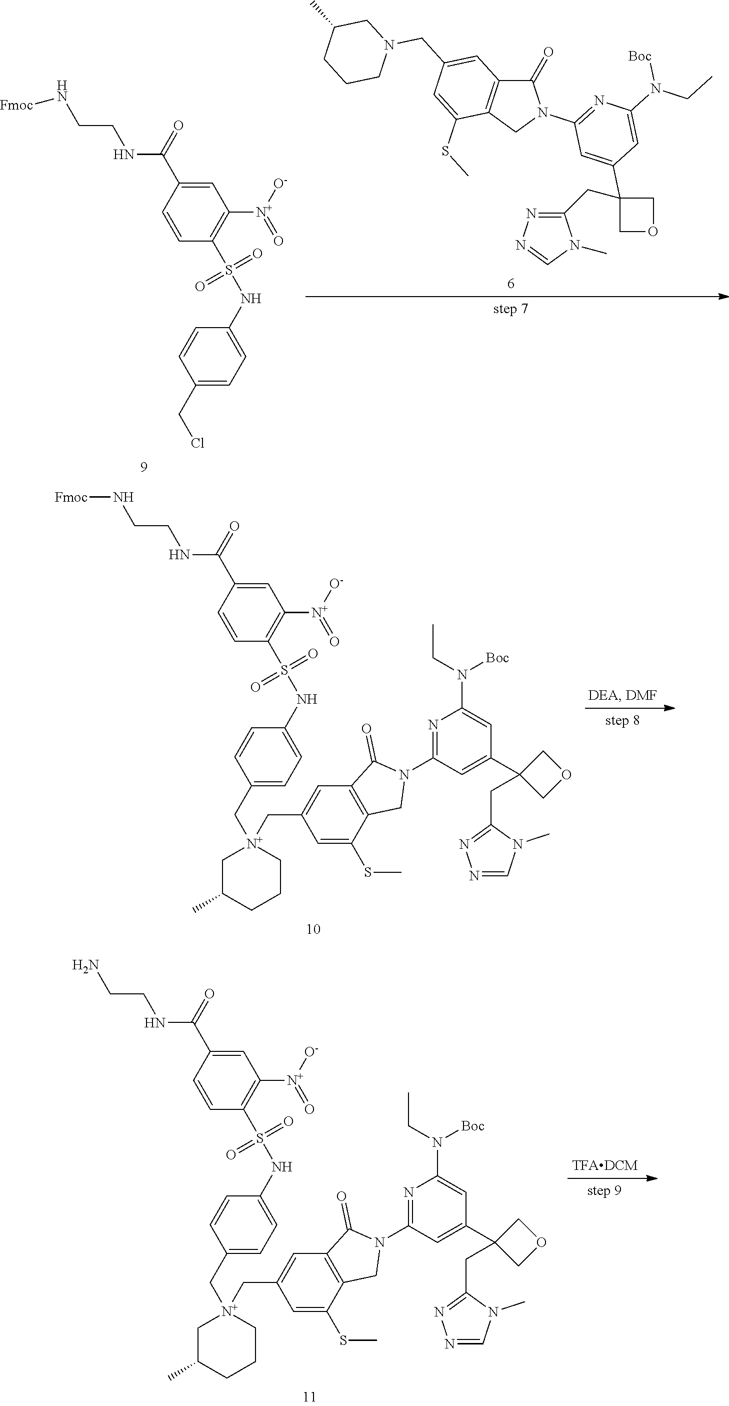

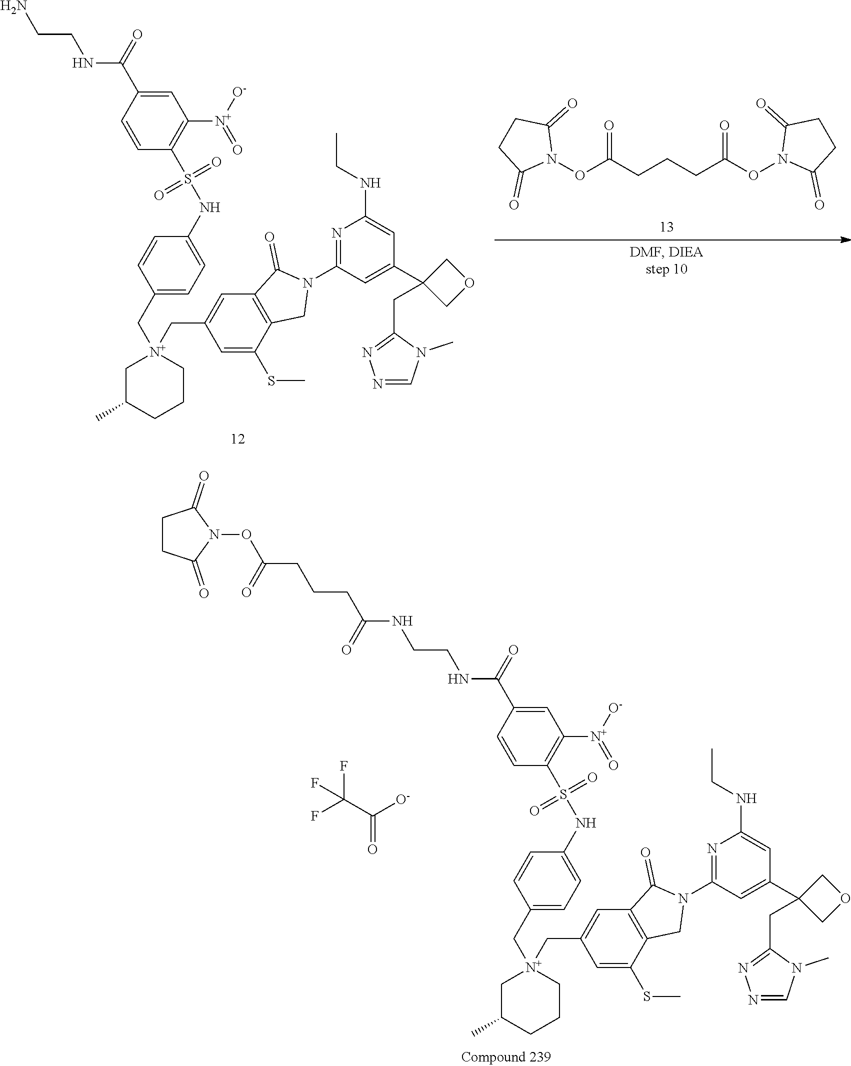 [SMILES: CCNc1cc(C2(Cc3nncn3C)COC2)cc(N2Cc3c(SC)cc(C[N+]4(Cc5ccc(NS(=O)(=O)c6ccc(C(=O)NCCN)cc6[N+](=O)[O-])cc5)CCC[C@H](C)C4)cc3C2=O)n1 ; O=C(CCCC(=O)ON1C(=O)CCC1=O)ON1C(=O)CCC1=O ; CCNc1cc(C2(Cc3nncn3C)COC2)cc(N2Cc3c(SC)cc(C[N+]4(Cc5ccc(NS(=O)(=O)c6ccc(C(=O)NCCNC(=O)CCCC(=O)ON7C(=O)CCC7=O)cc6[N+](=O)[O-])cc5)CCC[C@H](C)C4)cc3C2=O)n1 ; O=C([O-])C(F)(F)F]

### Step 1. Synthesis of Compound 2

A mixture of 2,6-dichloro-4-{3-[(4-methyl-1,2,4-triazol-3-yl)methyl]oxetan-3-yl}pyridine (4 g, 13.37 mmol, 1 equiv) in ethylamine solution 2.0 M in THE (60 mL) was stirred for overnight at 90° C. LCMS indicated the reaction was completed. The mixture was allowed to cool down to room temperature. The resulting mixture was concentrated under reduced pressure. The residue was purified by silica gel column chromatography, eluted with CH₂Cl₂/MeOH (10:1) to afford 6-chloro-N-ethyl-4-{3-[(4-methyl-1,2,4-triazol-3-yl)methyl]oxetan-3-yl}pyridin-2-amine (2.5 g, 60%) as an off-white solid. LCMS (ESI, m/z): 308,310 [M+H]⁺

### Step 2: Synthesis of Compound 4

A solution of 4-bromo-6-{[(3S)-3-methylpiperidin-1-yl]methyl}-2,3-dihydroisoindol-1-one (1 g, 3.09 mmol, 1 equiv) in DMF (15 mL) was treated with (methylsulfanyl)sodium (325 mg, 4.64 mmol, 1.5 equiv) at 25° C. The solution was stirred for 1 h at 90° C. Desired product could be detected by LCMS. The reaction was allowed to cool down to room temperature. The reaction was purified by reverse flash chromatography with the following conditions: column, C18 silica gel, 80 g, 20-35 um; mobile phase, water with 0.5% NH₄HCO₃ and ACN (0% to 100% gradient in 50 min); detector, UV 254 nm. This resulted in 6-{[(3S)-3-methylpiperidin-1-yl]methyl}-4-(methylsulfanyl)-2,3-dihydroisoindol-1-one (600 mg, 67%) as a white solid. LCMS (ES, m/z): 291 [M+H]⁺.

### Step 3: Synthesis of Compound 5

To a stirred mixture of 6-chloro-N-ethyl-4-{3-[(4-methyl-1,2,4-triazol-3-yl)methyl]oxetan-3-yl}pyridin-2-amine (500 mg, 1.62 mmol, 1 equiv) and 6-{[(3S)-3-methylpiperidin-1-yl]methyl}-4-(methylsulfanyl)-2,3-dihydroisoindol-1-one (519 mg, 1.79 mmol, 1.1 equiv) in dioxane (5.00 mL) was added Cs₂CO₃ (1.06 g, 3.25 mmol, 2 equiv), Xantphos (376 mg, 0.65 mmol, 0.4 equiv) and Pd(OAc)₂ (73 mg, 0.32 mmol, 0.2 equiv) at room temperature under air atmosphere. The resulting mixture was stirred for 2 h at 120° C. under nitrogen atmosphere. LCMS indicated the reaction was completed. The reaction was allowed to cool down to room temperature and concentrated to dryness under vacuum. The residue was purified by silica gel column chromatography, eluted with CH₂Cl₂/MeOH (10:1) to afford 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (770 mg, 77%) as an off-white solid. LCMS (ES, m/z): 562 [M+H]⁺.

### Step 4: Synthesis of Compound 6

To a stirred solution of 2-[6-(ethylamino)-4-{3-[(4-methyl-1,2,4-triazol-3-yl)methyl]oxetan-3-yl}pyridin-2-yl]-6-{[(3S)-3-methylpiperidin-1-yl]methyl}-4-(methylsulfanyl)-3H-isoindol-1-one (610 mg, 1.09 mmol, 1 equiv) in THE (26 mL) were added TEA (440 mg, 4.34 mmol, 4 equiv) and DMAP (40 mg, 0.32 mmol, 0.3 equiv) and Boc₂O (950 mg, 4.34 mmol, 4 equiv) at room temperature. The resulting mixture was stirred for overnight at 60° C. LCMS indicated the reaction was completed. The mixture was allowed to cool down to room temperature. The resulting mixture was concentrated under reduced pressure. The crude product was purified by silica gel column chromatography, eluted with DCM/MeOH (95:5) to afford tert-butyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (220 mg, 31%) as a yellow solid. LCMS:(ES·m/z): 662 [M+1]⁺.

### Step 5: Synthesis of Compound 8

To a stirred mixture of 4-{[4-(hydroxymethyl)phenyl]sulfamoyl}-3-nitrobenzoic acid (1 g, 2.84 mmol, 1 equiv) in DMF (15 mL) was added HATU (1.62 g, 4.26 mmol, 1.50 equiv) in portions at 0° C. The resulting mixture was stirred for 30 min. Then 9H-fluoren-9-ylmethyl N-(2-aminoethyl)carbamate hydrochloride (1.09 g, 3.41 mmol, 1.20 equiv) and DIEA (1.83 g, 14.19 mmol, 5 equiv) was added into the above mixture at 0° C. The resulting mixture was stirred for 2 h at 0° C. LCMS indicated the reaction was completed. The reaction was purified by reverse flash chromatography with the following conditions: column, C18 silica gel, 80 g, 20-35 um; mobile phase, water with 0.1% TFA and ACN (0% to 100% gradient in 50 min); detector, UV 254 nm. The collected fraction was concentrated to afford 9H-fluoren-9-ylmethyl N-{2-[(4-{[4-(hydroxymethyl)phenyl]sulfamoyl}-3-nitrophenyl)formamido]ethyl}carbamate (1.1 g, 63%) as a yellow solid. LCMS (ESI, m/z): 639 [M+Na]f.

### Step 6: Synthesis of Compound 9

To a mixture of 9H-fluoren-9-ylmethyl N-{2-[(4-{[4-(hydroxymethyl)phenyl]sulfamoyl}-3-nitrophenyl)formamido]ethyl}carbamate (200 mg, 0.32 mmol, 1 equiv) and DMF (20 drops) in THE (4 mL) was added SOCl₂ (77 mg, 0.65 mmol, 2.00 equiv) in THE (0.4 mL) dropwise at 0° C. The resulting mixture was stirred for 30 min at 25° C. LCMS indicated the reaction was completed. The reaction was lyophilized to afford crude 9H-fluoren-9-ylmethyl N-{2-[(4-{[4-(chloromethyl)phenyl]sulfamoyl}-3-nitrophenyl)formamido]ethyl}carbamate (220 mg, crude) as a yellow solid. LCMS (ESI, m/z): 657 [M+Na]⁺.

### Step 7: Synthesis of Compound 10

To a mixture of tert-butyl N-ethyl-N-(4-{3-[(4-methyl-1,2,4-triazol-3-yl)methyl]oxetan-3-yl}-6-(6-{[(3S)-3-methylpiperidin-1-yl]methyl}-4-(methylsulfanyl)-1-oxo-3H-isoindol-2-yl)pyridin-2-yl)carbamate (130 mg, 0.20 mmol, 1 equiv) and KI (65 mg, 0.40 mmol, 2.00 equiv) in DMF (3 mL) was added 9H-fluoren-9-ylmethyl N-{2-[(4-{[4-(chloromethyl)phenyl]sulfamoyl}-3-nitrophenyl)formamido]ethyl}carbamate (124 mg, 0.20 mmol, 1.00 equiv) in DMF (1 mL) in dropwise at 0° C. The resulting mixture was stirred at 50° C. for 4 h. LCMS indicated the reaction was completed. The reaction was allowed to cool down to room temperature. The reaction was purified by reverse flash chromatography with the following conditions: column, C18 silica gel, 80 g, 20-35 um; mobile phase, water with 0.05% TFA and ACN (0% to 100% gradient in 50 min); detector, UV 254 nm. The collected fraction was lyophilized to afford (3S)-1-[(2-{6-[(tert-butoxycarbonyl)(ethyl)amino]-4-{3-[(4-methyl-1,2,4-triazol-3-yl)methyl]oxetan-3-yl}pyridin-2-yl}-7-(methylsulfanyl)-3-oxo-1H-isoindol-5-yl)methyl]-1-[(4-{4-[(2-{[(9H-fluoren-9-ylmethoxy)carbonyl]amino}ethyl)carbamoyl]-2-nitrobenzenesulfonamido}phenyl)methyl]-3-methylpiperidin-1-ium (100 mg, 40%) as a yellow solid. LCMS (ESI, m/z): 1260.80 [M]⁺.

### Step 8: Synthesis of Compound 11

To a mixture of (3S)-1-[(2-{6-[(tert-butoxycarbonyl)(ethyl)amino]-4-{3-[(4-methyl-1,2,4-triazol-3-yl)methyl]oxetan-3-yl}pyridin-2-yl}-7-(methylsulfanyl)-3-oxo-1H-isoindol-5-yl)methyl]-1-[(4-{4-[(2-{[(9H-fluoren-9-ylmethoxy)carbonyl]amino}ethyl)carbamoyl]-2-nitrobenzenesulfonamido}phenyl)methyl]-3-methylpiperidin-1-ium (125 mg, 0.099 mmol, 1 equiv) in DMF (4 mL) was added diethylamine (1 mL) at 25° C. The resulting mixture was stirred for 1 h at 25° C. LCMS indicated the reaction was completed. The residue was purified by reverse flash chromatography with the following conditions: column, C18 silica gel, 80 g, 20-35 um; mobile phase, water with 0.05% TFA and ACN (0% to 100% gradient in 50 min); detector, UV 254 nm. The collected fraction was lyophilized to afford (3S)-1-[(4-{4-[(2-aminoethyl)carbamoyl]-2-nitrobenzenesulfonamido}phenyl)methyl]-1-[(2-{6-[(tert-butoxycarbonyl)(ethyl)amino]-4-{3-[(4-methyl-1,2,4-triazol-3-yl)methyl]oxetan-3-yl}pyridin-2-yl}-7-(methylsulfanyl)-3-oxo-1H-isoindol-5-yl)methyl]-3-methylpiperidin-1-ium (80 mg, 77%) as a yellow solid. LCMS (ESI, m/z): 1038.6 [M]⁺.

### Step 9: Synthesis of Compound 12

To a mixture of (3S)-1-[(4-{4-[(2-aminoethyl)carbamoyl]-2-nitrobenzenesulfonamido}phenyl)methyl]-1-[(2-{6-[(tert-butoxycarbonyl)(ethyl)amino]-4-{3-[(4-methyl-1,2,4-triazol-3-yl)methyl]oxetan-3-yl}pyridin-2-yl}-7-(methylsulfanyl)-3-oxo-1H-isoindol-5-yl)methyl]-3-methylpiperidin-1-ium (75 mg, 0.072 mmol, 1 equiv) in DCM (5 mL) was added TFA (2 mL) at 25° C. The resulting mixture was stirred for 1 h at 25° C. LCMS indicated the reaction was completed. The solvent was removed under reduced pressure. The residue was lyophilized to afford crude (3S)-1-[(4-{4-[(2-aminoethyl)carbamoyl]-2-nitrobenzenesulfonamido}phenyl)methyl]-1-({2-[6-(ethylamino)-4-{3-[(4-methyl-1,2,4-triazol-3-yl)methyl]oxetan-3-yl}pyridin-2-yl]-7-(methylsulfanyl)-3-oxo-1H-isoindol-5-yl}methyl)-3-methylpiperidin-1-ium (85 mg, crude) as a yellow solid. LCMS (ESI, m/z): 938.6 [M]⁺.

### Step 10: Synthesis of Compound 239

To a stirred mixture of (3S)-1-[(4-{4-[(2-aminoethyl)carbamoyl]-2-nitrobenzenesulfonamido}phenyl)methyl]-1-({2-[6-(ethylamino)-4-{3-[(4-methyl-1,2,4-triazol-3-yl)methyl]oxetan-3-yl}pyridin-2-yl]-7-(methylsulfanyl)-3-oxo-1H-isoindol-5-yl}methyl)-3-methylpiperidin-1-ium (75 mg, 0.080 mmol, 1 equiv) and DIEA (41 mg, 0.32 mmol, 4 equiv) in DMF (1.5 mL) was added bis(2,5-dioxopyrrolidin-1-yl) pentanedioate (26 mg, 0.080 mmol, 1 equiv) at 25° C. The resulting mixture was stirred for 1 h at 25° C. LCMS indicated the reaction was completed. The reaction mixture was purified with prep-HPLC with following conditions: Column: XBridge Prep Phenyl OBD C18 Column, 19*250 mm, 5 m; Mobile Phase A: Water (0.05% TFA), Mobile Phase B: ACN; Flow rate: 25 mL/min mL/min; Gradient: 10% B to 40% B in 20 min; Wave Length: 254 nm/220 nm; RT1 (min): 22.57. The collected fraction was lyophilized to afford (3S)-1-[(4-{4-[(2-{5-[(2,5-dioxopyrrolidin-1-yl)oxy]-5-oxopentanamido}ethyl)carbamoyl]-2-nitrobenzenesulfonamido}phenyl)methyl]-1-({2-[6-(ethylamino)-4-{3-[(4-methyl-1,2,4-triazol-3-yl)methyl]oxetan-3-yl}pyridin-2-yl]-7-(methylsulfanyl)-3-oxo-1H-isoindol-5-yl}methyl)-3-methylpiperidin-1-ium; trifluoroacetate (11.8 mg, 10.6%) as a yellow solid. LCMS (ESI, m/z): 1149.5 [M]⁺. ¹H NMR (400 MHz, DMSO-d₆) δ 11.06 (s, 1H), 10.05 (br s, 1H), 9.91 (s, 1H), 8.91 (t, J=5.2 Hz, 1H), 8.32 (s, 1H), 8.15 (d, J=8.0 Hz, 1H), 8.07 (d, J=8.0 Hz, 1H), 7.98 (t, J=5.6 Hz, 1H), 7.71 (d, J=6.8 Hz, 2H), 7.37-7.30 (m, 3H), 7.13 (d, J=8.4 Hz, 2H), 6.11 (s, 1H), 5.47 (s, 2H), 4.91 (s, 2H), 4.83-4.77 (m, 4H), 4.43 (s, 2H), 3.72 (s, 2H), 3.56 (s, 3H), 3.41-3.16 (m, 8H), 2.95-2.82 (m, 5H), 2.70-2.60 (m, 6H), 2.18 (t, J=7.2 Hz, 2H), 1.89-1.62 (m, 6H), 1.17 (t, J=7.2 Hz, 3H), 1.09-1.02 (m, 1H), 0.88 (d, J=6.4 Hz, 3H).

### Synthesis of Compound 240

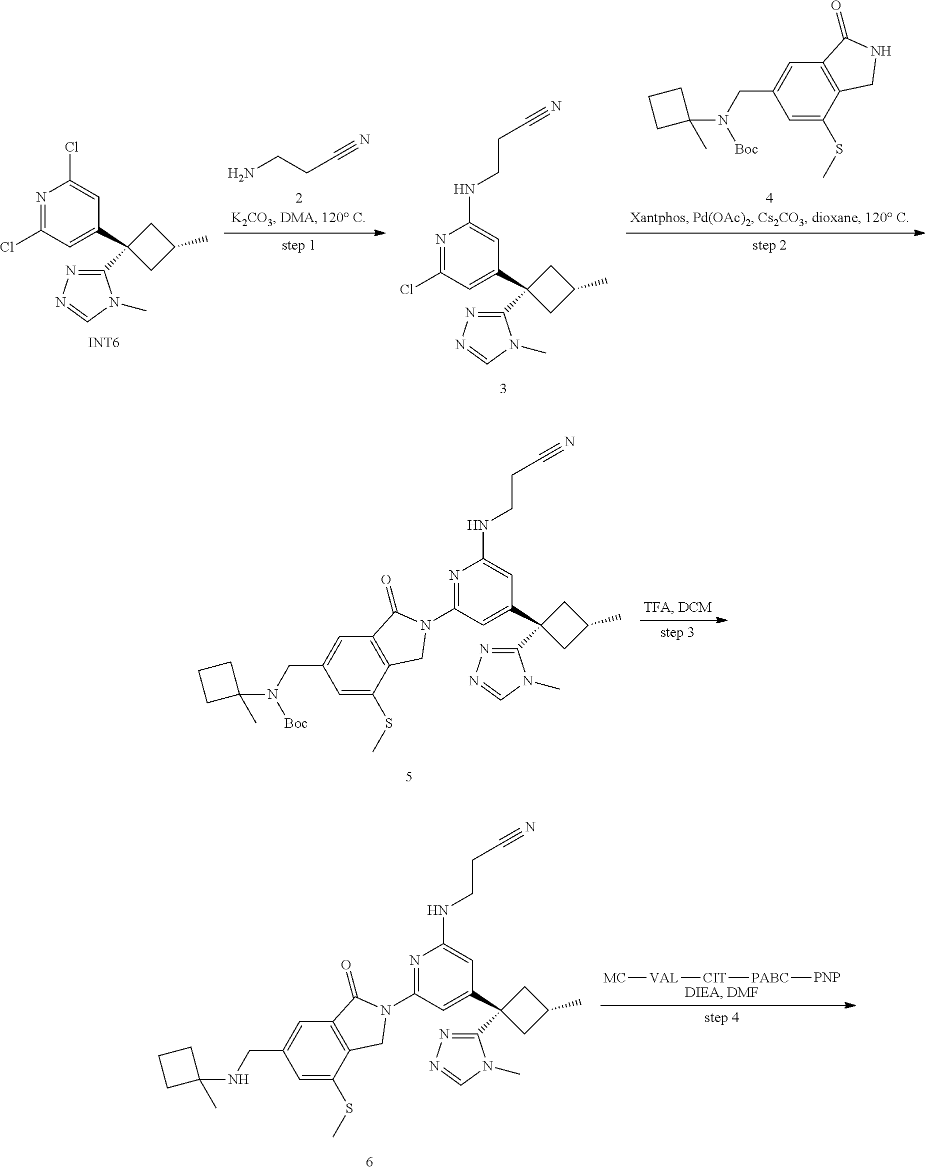

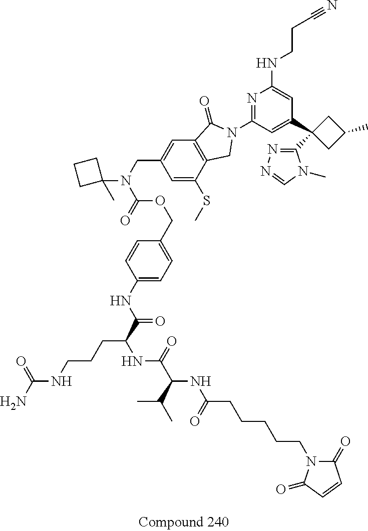 [SMILES: CSc1cc(CN(C(=O)OCc2ccc(NC(=O)[C@H](CCCNC(N)=O)NC(=O)[C@@H](NC(=O)CCCCCN3C(=O)C=CC3=O)C(C)C)cc2)C2(C)CCC2)cc2c1CN(c1cc([C@]3(c4nncn4C)C[C@@H](C)C3)cc(NCCC#N)n1)C2=O]

Step 1. Synthesis of Compound 3

A solution of 2,6-dichloro-4-[(1r,3s)-3-methyl-1-(4-methyl-1,2,4-triazol-3-yl)cyclobutyl]pyridine (500 mg, 1.68 mmol, 1.0 equiv) in DMA (10 mL) was treated with 3-aminopropionitrile (2.36 g, 33.64 mmol, 20.0 equiv), and K₂CO₃ (465 mg, 3.36 mmol, 2.0 equiv). The resulting mixture was stirred for 16 h at 120° C. under nitrogen atmosphere. Desired product could be detected by LCMS. The reaction mixture was allowed to cool down to room temperature and purified by reverse flash chromatography with the following conditions: column, Cis, 40 g, 20-35 um; mobile phase, water with 0.0800 NH₄HCO₃ and ACN (00% to 50% gradient in 50 min); detector, UV 254 nm. The collected fractions were lyophilized to afford 3-({6-chloro-4-[(1r,3s)-3-methyl-1-(4-methyl-1,2,4-triazol-3-yl)cyclobutyl]pyridin-2-yl}amino)propanenitrile (310 mg, 48%) as an off-white solid. LCMS (ESI·m/z): 331.333 [M+H]⁺

### Step 2: Synthesis of Compound 5

A solution of 3-({6-chloro-4-[(1r,3s)-3-methyl-1-(4-methyl-1,2,4-triazol-3-yl)cyclobutyl]pyridin-2-yl}amino)propanenitrile (200 mg, 0.61 mmol, 1.0 equiv) in dioxane (12 mL) was treated with tert-butyl N-(1-methylcyclobutyl)-N-{[7-(methylsulfanyl)-3-oxo-1,2-dihydroisoindol-5-yl]methyl}carbamate (341 mg, 0.91 mmol, 1.5 equiv), Pd(OAc)₂ (54 mg, 0.24 mmol, 0.4 equiv), [5-(diphenylphosphanyl)-9,9-dimethyl-9H-xanthen-4-yl]diphenylphosphane (140 mg, 0.24 mmol, 0.4 equiv) and Cs₂CO₃ (1.58 g, 4.84 mmol, 8.0 equiv). The resulting mixture was stirred for 1.5 h at 100° C. under nitrogen atmosphere. Desired product could be detected by LCMS. The resulting mixture was concentrated under reduced pressure. The residue was purified by silica gel column chromatography, eluted with CH₂Cl₂/MeOH (20:1) to afford tert-butyl N-[(2-{6-[(2-cyanoethyl)amino]-4-[(1r,3s)-3-methyl-1-(4-methyl-1,2,4-triazol-3-yl)cyclobutyl]pyridin-2-yl}-7-(methylsulfanyl)-3-oxo-1H-isoindol-5-yl)methyl]-N-(1-methylcyclobutyl)carbamate (120 mg, 26%) as an off-white solid. LCMS (ESI, m/z): 671 [M+H]⁺.

### Step 3: Synthesis of Compound 6

A solution of tert-butyl N-[(2-{6-[(2-cyanoethyl)amino]-4-[(1r,3s)-3-methyl-1-(4-methyl-1,2,4-triazol-3-yl)cyclobutyl]pyridin-2-yl}-7-(methylsulfanyl)-3-oxo-1H-isoindol-5-yl)methyl]-N-(1-methylcyclobutyl)carbamate (40 mg, 0.060 mmol, 1.0 equiv) in DCM (3 mL) was treated with TFA (1 mL). The resulting mixture was stirred for 1 h at room temperature. Desired product could be detected by LCMS. The resulting mixture was stirred for 1 h at room temperature. Desired product could be detected by LCMS. The residue was submitted to Prep-HPLC purification (Column: XBridge Prep OBD C18 Column, 19*250 mm, 5 m; Mobile Phase A: water (0.05% TFA), Mobile Phase B: ACN; Flow rate: 25 mL/min mL/min; Gradient: 7% B to 47% B in 10 min; Wave Length: 254 nm/220 nm; RT1 (min): 9.77) and the collected fractions were lyophilized to afford 3-{[6-(6-{[(1-methylcyclobutyl)amino]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]amino}propanenitrile; trifluoroacetic acid (20.40 mg, 49%) as a white solid. LCMS (ESI, m/z): 571 [M+H]⁺.

### Step 4: Synthesis of Compound 240

A solution of 3-{[6-(6-{[(1-methylcyclobutyl)amino]methyl}-4-(methylsulfanyl)-1-oxo-3H-isoindol-2-yl)-4-[(1r,3s)-3-methyl-1-(4-methyl-1,2,4-triazol-3-yl)cyclobutyl]pyridin-2-yl]amino}propanenitrile (90 mg, 0.16 mmol, 1.0 equiv) in DMF (0.5 mL) was treated with {4-[(2S)-5-(carbamoylamino)-2-[(2S)-2-[6-(2,5-dioxo-2,5-dihydro-1H-pyrrol-1-yl)hexanamido]-3-methylbutanamido]pentanamido]phenyl}methyl 4-nitrophenyl carbonate (116 mg, 0.16 mmol, 1.0 equiv) and DIEA (102 mg, 0.79 mmol, 5.0 equiv). The resulting mixture was stirred for 16 h at 50° C. Desired product could be detected by LCMS. The reaction mixture was submitted to Prep-HPLC purification (Column: 254 nm/220 nm; Mobile Phase A: XBridge Prep OBD C18 Column, 30*150 mm, 5 m, Mobile Phase B: water (10 nmol/LNH₄HCO₃+0.05% NH₃·H₂O); Gradient: isocratic 60 mL/min; Wave Length: 254 nm/220 nm; RT1 (min): 35% B to 65% B in 20 min) and the collected fractions were lyophilized to afford {4-[(2S)-5-(carbamoylamino)-2-[(2S)-2-[6-(2,5-dioxopyrrol-1-yl)hexanamido]-3-methylbutanamido]pentanamido]phenyl}methyl N-[(2-{6-[(2-cyanoethyl)amino]-4-[(1r,3s)-3-methyl-1-(4-methyl-1,2,4-triazol-3-yl)cyclobutyl]pyridin-2-yl}-7-(methylsulfanyl)-3-oxo-1H-isoindol-5-yl)methyl]-N-(1-methylcyclobutyl)carbamate (20.70 mg, 11%) as a white solid. LCMS (ESI, m/z): 1169 [M+H]⁺. ¹H NMR (400 MHz, DMSO-d₆) δ 10.01 (s, 1H), 8.35 (s, 1H), 8.09 (d, J=6.4 Hz, 1H), 7.81-7.79 (m, 2H), 7.59 (br s, 2H), 7.43-7.41 (m, 2H), 7.34-7.32 (m, 1H), 7.11 (d, J=5.2 Hz, 1H), 7.00 (s, 2H), 6.18 (s, 1H), 5.98-5.96 (m, 1H), 5.41 (s, 2H), 5.03 (s, 2H), 4.92 (s, 2H), 4.42-4.40 (m, 3H), 4.19 (t, J=7.6 Hz, 1H), 3.54-3.52 (m, 2H), 3.34-3.32 (m, 2H), 3.25 (s, 3H), 3.08-2.98 (m, 2H), 2.84 (t, J=7.6 Hz, 2H), 2.79-2.69 (m, 2H), 2.55-2.52 (m, 3H), 2.18-2.11 (m, 4H), 2.01-1.83 (m, 3H), 1.68-151 (m, 4H), 1.48-1.45 (m, 5H), 1.39-1.29 (m, 4H), 1.23-1.18 (m, 3H), 1.09-1.08 (m, 3H), 0.86-0.81 (m, 7H).

### Synthesis of Compound 241

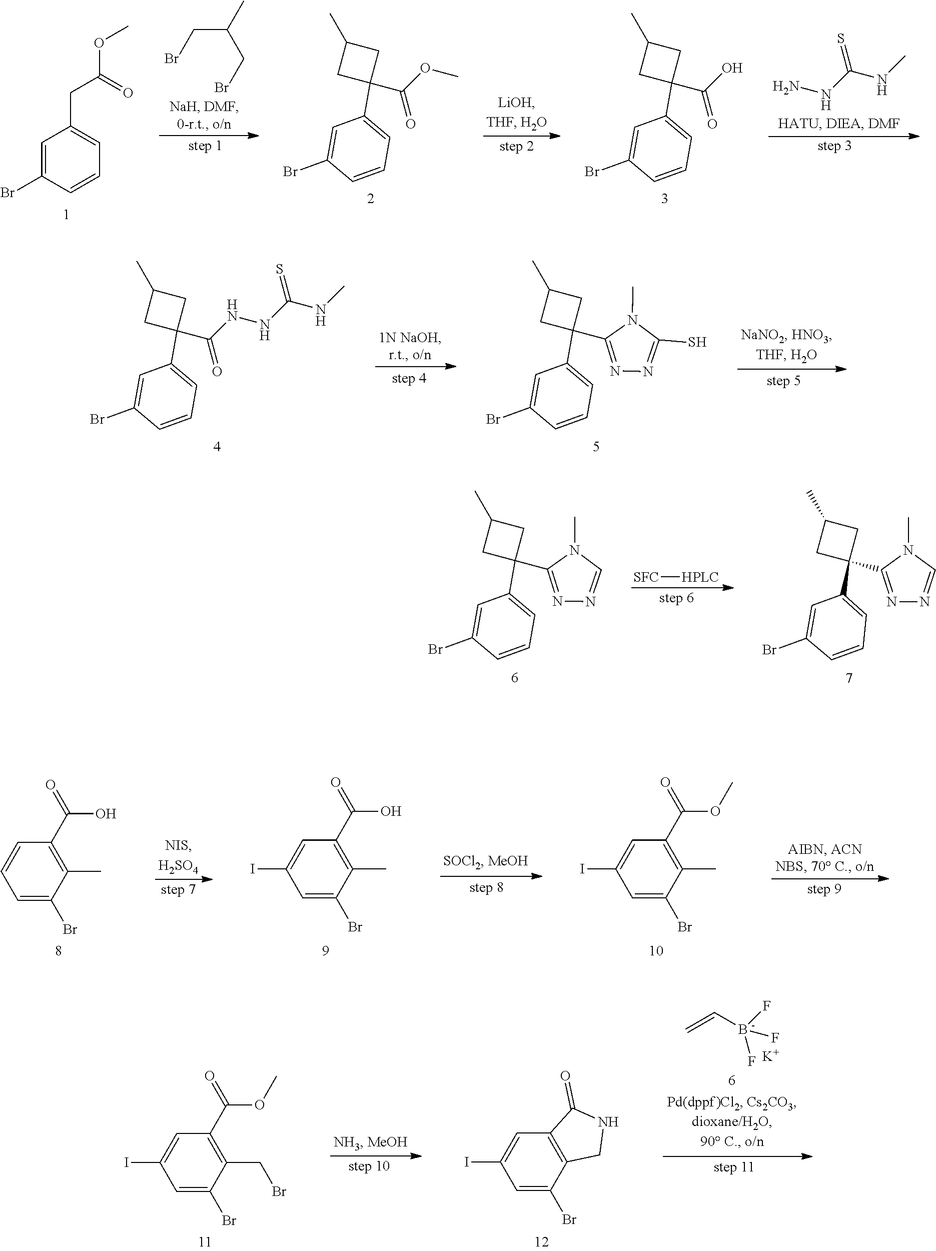

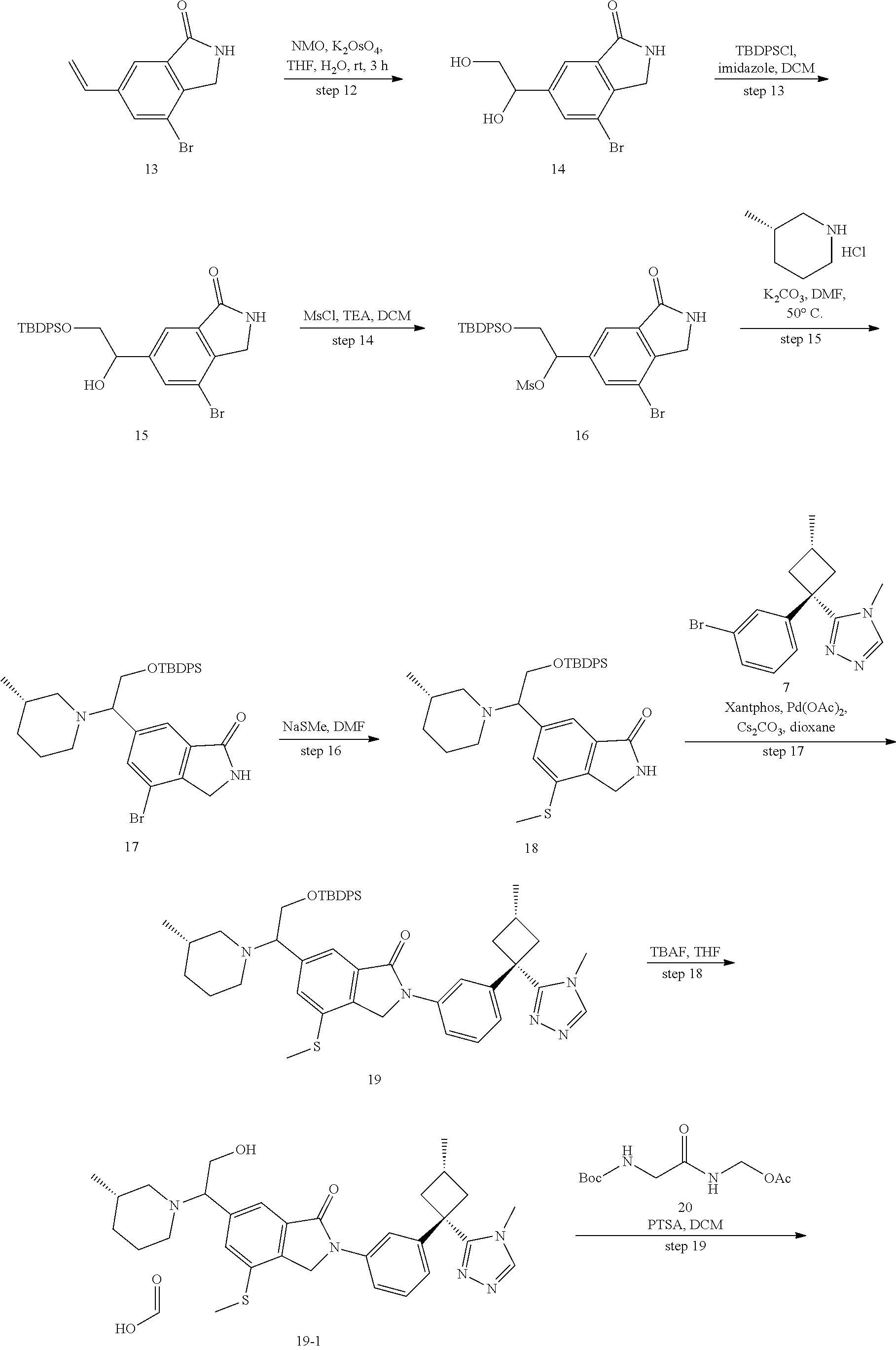

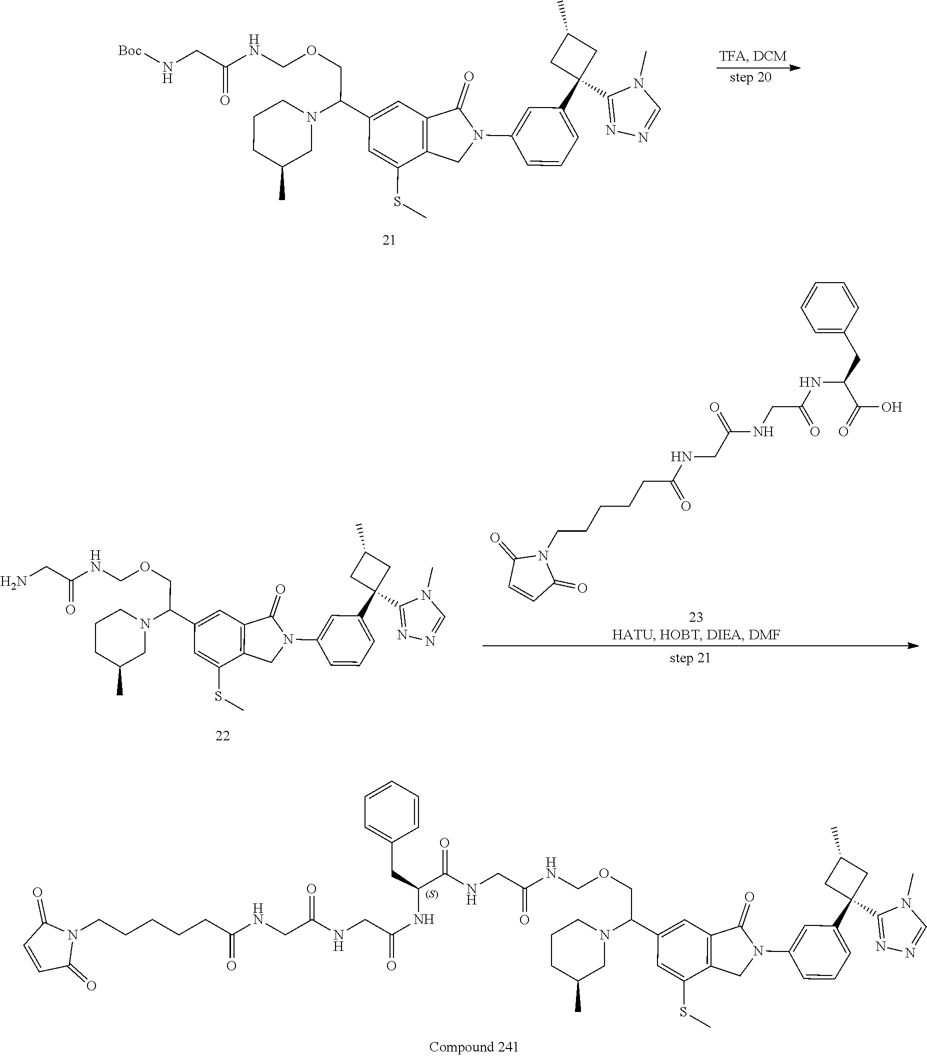

### Step 1: Synthesis of Compound 2

To a stirred solution of methyl 2-(3-bromophenyl)acetate (100 g, 436.54 mmol, 1 equiv) in DMF (1 L) was added NaH (26.19 g, 1091.35 mmol, 1.5 equiv, 60%) in portions at 0° C. under air atmosphere. The resulting mixture was stirred for 30 min at 0° C. under air atmosphere. To the above mixture was added 1,3-dibromo-2-methylpropane (103.68 g, 480.19 mmol, 1.1 equiv) in portions over at 0° C. The resulting mixture was stirred for additional overnight at room temperature. LCMS indicated the reaction was completed. The reaction was quenched by the addition of sat. NH₄Cl (aq.) (1000 mL) at 0° C. The resulting mixture was extracted with EtOAc (3×700 mL). The combined organic layers were washed with brine (3×200 mL), dried over anhydrous Na₂SO₄. After filtration, the filtrate was concentrated under reduced pressure. The residue was purified by silica gel column chromatography, eluted with PE/EA (5:1) to afford methyl 1-(3-bromophenyl)-3-methylcyclobutane-1-carboxylate (100 g, 81%) as a yellow oil. LCMS:(ES·m/z):283,284[M+H]⁺.

### Step 2: Synthesis of Compound 3

To a stirred solution of methyl 1-(3-bromophenyl)-3-methylcyclobutane-1-carboxylate (100 g, 353.15 mmol, 1 equiv) in THE (100 mL) was added LiGH (25.37 g, 1059.45 mmol, 3 equiv) in H₂O (1000 mL) at 0° C. The resulting mixture was stirred for overnight at room temperature under nitrogen atmosphere. LCMS indicated the reaction was completed. The resulting mixture was concentrated under vacuum. The mixture was acidified to pH 6 with conc. HCl. The resulting mixture was extracted with EtOAc (3×200 mL). The combined organic layers were washed with brine (3×100 mL), dried over anhydrous Na₂SO₄. After filtration, the filtrate was concentrated under reduced pressure. This resulted in 1-(3-bromophenyl)-3-methylcyclobutane-1-carboxylic acid (30 g, 32%) as a yellow solid. LCMS (ESI, m/z):267,269[M−H]⁻.

### Step 3: Synthesis of Compound 4

To a stirred solution of 1-(3-bromophenyl)-3-methylcyclobutane-1-carboxylic acid (16.5 g, 61.31 mmol, 1 equiv) and 1-amino-3-methylthiourea (7.74 g, 73.57 mmol, 1.2 equiv) in DMF (170 mL) was added HATU (34.97 g, 91.96 mmol, 1.5 equiv) and DIEA (15.85 g, 122.61 mmol, 2 equiv) at room temperature. The resulting mixture was stirred for overnight at room temperature under nitrogen atmosphere. LCMS indicated the reaction was completed. The reaction mixture was used to next step without any treatment. LCMS:(ES·m/z):356,358[M+H]⁺.

### Step 4: Synthesis of Compound 5

To the above mixture was added NaOH (170 mL) at room temperature. The resulting mixture was stirred for additional overnight at room temperature. LCMS indicated the reaction was completed. The mixture was acidified to pH 6 with 1N HCl. The resulting mixture was extracted with EtOAc (3×100 mL). The combined organic layers were washed with brine (5×100 mL), dried over anhydrous Na₂SO₄. After filtration, the filtrate was concentrated under reduced pressure. The residue was purified by silica gel column chromatography, eluted with CH₂Cl₂/MeOH (10:1) to afford 5-[1-(3-bromophenyl)-3-methylcyclobutyl]-4-methyl-1,2,4-triazole-3-thiol (4.1 g, 20%) as a yellow oil. LCMS:(ES·m/z):338,340[M+H]⁺.

### Step 5: Synthesis of Compound 6

To a stirred mixture of 5-[1-(3-bromophenyl)-3-methylcyclobutyl]-4-methyl-1,2,4-triazole-3-thiol (22 g, 65.03 mmol, 1.00 equiv) in THE (22.02 mL) was added NaNO₂ (22.44 g, 325.18 mmol, 5 equiv) in portions at 0 degrees C. under nitrogen atmosphere. The mixture was added HNO₃ (325.18 mL, 325.18 mmol, 5 equiv, 1N) dropwise at 0 degrees C. under nitrogen atmosphere. LCMS indicated the reaction was completed. The mixture was basified to pH 8 with saturated NaHCO₃ (aq.). The resulting mixture was extracted with EtOAc (3×200 mL). The combined organic layers were washed with water (4×80 mL). The resulting mixture was concentrated under reduced pressure. The residue was purified by silica gel column chromatography, eluted with CH₂Cl₂/MeOH (10:1) to afford 3-[1-(3-bromophenyl)-3-methylcyclobutyl]-4-methyl-1,2,4-triazole (10 g, 50%) as an off-white solid. LCMS:(ES·m/z):306,308[M+H]⁺.

### Step 6: Synthesis of Compound 7

The product (10 g) was separated by Prep-SFC with the following conditions (Column: CHIRALPAK ID 2*25 cm, Sum; Mobile Phase A: CO₂, Mobile Phase B: IPA (1%-2M-NH₃-MeOH); Flow rate: 55 mL/min; Gradient: isocratic 20% B; Column Temperature (° C.): 35; Back Pressure (bar): 100; Wave Length: 220 nm; RT1 (min): 12.13; RT2 (min): 13.8; Sample Solvent: MEOH; Injection Volume: 0.8 mL. The second fraction (RT13.8 min) was concentrated to afford 4-methyl-3-[(1r,3s)-1-(3-bromophenyl)-3-methylcyclobutyl]-1,2,4-triazole (6 g, 60%) as an off-white solid. ¹H NMR (300 MHz, Chloroform-d) δ 8.00 (s, 1H), 7.53 (s, 1H), 7.40-7.37 (s, 1H), 7.24-7.21 (m, 2H), 3.19 (s, 3H), 2.82-2.78 (m, 2H), 2.67-2.63 (m, 3H), 1.22-1.12 (m, 3H).

### Step 7: Synthesis of Compound 9

To a stirred solution of NIS (57.54 g, 255.76 mmol, 1.1 equiv) in H2SO₄ (200 mL) was stirred for 40 min at room temperature under nitrogen atmosphere. To the above mixture was added 3-bromo-2-methylbenzoic acid (50 g, 232.51 mmol, 1 equiv) in H2SO₄ (200 mL) dropwise at 0° C. and stirred for 3 h, The resulting mixture was stirred for additional overnight at room temperature. LCMS indicated complete reaction. LCMS indicated complete reaction. The reaction was pour into Water/Ice at 0° C. The precipitated solids were collected by filtration and washed with Et₂O (3×20 mL). The residue was purified by trituration with DCM (100 mL). The precipitated solids were collected by filtration and washed with DCM (3×50 mL). The solid was dried under infrared to afford 3-bromo-5-iodo-2-methylbenzoic acid (54.2 g, 68%) as an off-white solid. LCMS (ESI, ms):339,341[M−H]⁻. ¹H NMR (300 MHz, DMSO-d₆) δ 8.13 (d, J=1.8 Hz, 1H), 7.99 (d, J=1.8 Hz, 1H), 2.47 (s, 3H).

### Step 8: Synthesis of Compound 10

To a stirred solution of 3-bromo-5-iodo-2-methylbenzoic acid (30 g, 87.99 mmol, 1 equiv) in MeOH (300 mL) was added SOCl₂ (52.34 g, 439.96 mmol, 5 equiv) dropwise at 0° C. under nitrogen atmosphere. The resulting mixture was stirred for 3 h at 70° C. under nitrogen atmosphere. LCMS indicated the reaction was completed. The resulting mixture was concentrated under reduced pressure. The reaction was quenched with sat. NaHCO₃ (aq.) at 0° C. The resulting mixture was extracted with EtOAc (3×100 mL). The combined organic layers were washed with brine (100 mL), dried over anhydrous Na₂SO₄. After filtration, the filtrate was concentrated under reduced pressure. This resulted in methyl 3-bromo-5-iodo-2-methylbenzoate (29.9 g, 96%) as an off-white solid. No MS signal. ¹H NMR (400 MHz, Chloroform-d) δ 8.03 (d, J=1.9 Hz, 1H), 8.02 (d, J=1.9 Hz, 1H), 3.90 (s, 3H), 2.56 (s, 3H).

### Step 9: Synthesis of Compound 11

To a stirred solution of methyl 3-bromo-5-iodo-2-methylbenzoate (15 g, 42.25 mmol, 1 equiv) and NBS (9.03 g, 50.70 mmol, 1.2 equiv) in ACN (150 mL) was added AIBN (3.47 g, 21.12 mmol, 0.5 equiv) in portions at 70° C. under nitrogen atmosphere. LCMS & TLC showed the reaction was completed. no MS signal. The resulting mixture was concentrated under reduced pressure. The residue was purified by silica gel column chromatography, eluted with PE/EA (6:1) to afford methyl 3-bromo-2-(bromomethyl)-5-iodobenzoate (16.5 g, 90%) as a white solid. ¹H NMR (300 MHz, Chloroform-d) δ 8.20 (d, J=1.9 Hz, 1H), 8.11 (d, J=1.8 Hz, 1H), 5.06 (s, 2H), 1.73 (s, 3H).

### Step 10: Synthesis of Compound 12

A mixture of methyl 3-bromo-2-(bromomethyl)-5-iodobenzoate (51.5 g, 118.70 mmol, 1 equiv) and NH₃ (7N in MeOH, 500 mL) was stirred for 2 h at 25° C. LCMS indicated the reaction was completed. The solvent was removed under reduced pressure. The residue was used for next step without further purification. LCMS (ESI, ms): 338,340 [M+H]⁺.

### Step 11: Synthesis of Compound 13

To a stirred solution of 4-bromo-6-iodo-2,3-dihydroisoindol-1-one (48 g, 142.03 mmol, 1 equiv) and potassium ethenyltrifluoroboranuide (15.22 g, 113.62 mmol, 0.8 equiv) in 1,4-dioxane (500 mL) and H₂O (100 mL) was added Cs₂CO₃ (92.56 g, 284.07 mmol, 2 equiv) and Pd(dppf)Cl₂ (10.39 g, 14.20 mmol, 0.1 equiv) at room temperature under nitrogen atmosphere. The resulting mixture was stirred for 1 h at 90° C. under nitrogen atmosphere. LCMS indicated the reaction was completed. The mixture was allowed to cool down to room temperature. The resulting mixture was concentrated under reduced pressure. The residue was purified by silica gel column chromatography, eluted with CH₂Cl₂/MeOH (12:1) to afford 4-bromo-6-ethenyl-2,3-dihydroisoindol-1-one (11 g, 32%) as a brown solid. LCMS (ESI, ms):238,240[M+H]⁺.

### Step 12: Synthesis of Compound 14

To a stirred solution of 4-bromo-6-ethenyl-2,3-dihydroisoindol-1-one (20 g, 84.00 mmol, 1 equiv) and NMO (29.52 g, 252.00 mmol, 3.00 equiv) in THE (200 mL) and water (100 mL) was added K₂OsO₄·2H₂O (0.15 g, 0.42 mmol, 0.05 equiv) at room temperature under nitrogen atmosphere. The resulting mixture was stirred for 3 h at room temperature under nitrogen atmosphere. LCMS indicated the reaction was completed. THE was removed under reduced pressure. The residue was purified by reverse flash chromatography with the following conditions: column, C18 silica gel, 80 g, 20-35 um; mobile phase, water with 0.1% TFA and ACN (0% to 100% gradient in 50 min); detector, UV 254 nm. The collected fraction was lyophilized to afford 4-bromo-6-(1,2-dihydroxyethyl)-2,3-dihydroisoindol-1-one (6.7 g, 29%) as a brown solid. LCMS (ESI, ms): 272,274 [M+H]⁺.

### Step 13: Synthesis of Compound 15

To a stirred solution of 4-bromo-6-(1,2-dihydroxyethyl)-2,3-dihydroisoindol-1-one (5 g, 18.37 mmol, 1 equiv) in DCM (50 mL) was added imidazole (2.50 g, 36.75 mmol, 2 equiv) and TBDPSCl (5.05 g, 18.37 mmol, 1 equiv) dropwise at room temperature under air atmosphere. The resulting mixture was stirred for 2 h at room temperature under air atmosphere. LCMS indicated the reaction was completed. The resulting mixture was concentrated under reduced pressure. The residue was purified by silica gel column chromatography, eluted with PE/EA (1:1) to afford 4-bromo-6-{2-[(tert-butyldiphenylsilyl)oxy]-1-hydroxyethyl}-2,3-dihydroisoindol-1-one (3.6 g, 38%) as a white solid. LCMS:(ES·m/z):510,512[M+H]⁺.

### Step 14: Synthesis of Compound 16

To a stirred solution of 4-bromo-6-{2-[(tert-butyldiphenylsilyl)oxy]-1-hydroxyethyl}-2,3-dihydroisoindol-1-one (3.5 g, 6.85 mmol, 1 equiv) in DCM (70 mL) was added MsCl (0.94 g, 8.22 mmol, 1.2 equiv) and TEA (2.08 g, 20.56 mmol, 3 equiv) dropwise at room temperature under air atmosphere. The resulting mixture was stirred for 2 h at room temperature under air atmosphere. LCMS indicated the reaction was completed. The resulting mixture was concentrated under reduced pressure. The resulting mixture was used in the next step directly without further purification. LCMS:(ES·m/z):588,590[M+H]⁺.

### Step 15: Synthesis of Compound 17

To a stirred solution of 1-(7-bromo-3-oxo-1,2-dihydroisoindol-5-yl)-2-[(tert-butyldiphenylsilyl)oxy]ethyl methanesulfonate (3.5 g, 5.94 mmol, 1 equiv) and (3S)-3-methylpiperidine (0.71 g, 7.13 mmol, 1.2 equiv) in DMF (40 mL) was added K₂CO₃ (2.05 g, 14.86 mmol, 2.5 equiv) in portions at room temperature under air atmosphere. The resulting mixture was stirred for overnight at 50° C. under air atmosphere. LCMS indicated the reaction was completed. The mixture was allowed to cool down to room temperature. The reaction was quenched by the addition of Water/Ice (200 mL) at 0° C. The resulting mixture was extracted with EtOAc (3×100 mL). The combined organic layers were washed with brine (3×5 mL), dried over anhydrous Na₂SO₄. After filtration, the filtrate was concentrated under reduced pressure. The residue was purified by silica gel column chromatography, eluted with PE/EA (1:1) to afford 4-bromo-6-{2-[(tert-butyldiphenylsilyl)oxy]-1-[(3S)-3-methylpiperidin-1-yl]ethyl}-2,3-dihydroisoindol-1-one (2.8 g, 79%) as a yellow solid. LCMS:(ES·m/z):591,593[M+H]⁺.

### Step 16: Synthesis of Compound 18

To a stirred solution of 4-bromo-6-{2-[(tert-butyldiphenylsilyl)oxy]-1-[(3S)-3-methylpiperidin-1-yl]ethyl}-2,3-dihydroisoindol-1-one (2 g, 3.38 mmol, 1 equiv) in DMF (20 mL) was added (methylsulfanyl)sodium (0.36 g, 5.07 mmol, 1.5 equiv) in portions at room temperature under air atmosphere. The resulting mixture was stirred for 2 h at 100° C. LCMS indicated the reaction was completed. The mixture was allowed to cool down to room temperature. The residue was purified by reversed-phase flash chromatography with the following conditions: column, C18 silica gel; mobile phase, MeCN in Water (0.05% TFA), 0% to 50% gradient in 40 min; detector, UV 254 nm. The collected fraction was concentrated to afford 6-{2-[(tert-butyldiphenylsilyl)oxy]-1-[(3S)-3-methylpiperidin-1-yl]ethyl}-4-(methylsulfanyl)-2,3-dihydroisoindol-1-one (600 mg, 32%) as a yellow oil. LCMS:(ES·m/z):559[M+H]⁺.

### Step 17: Synthesis of Compound 19

To a stirred mixture of 6-{2-[(tert-butyldiphenylsilyl)oxy]-1-[(3S)-3-methylpiperidin-1-yl]ethyl}-4-(methylsulfanyl)-2,3-dihydroisoindol-1-one (600 mg, 1.074 mmol, 1 equiv) and 4-methyl-3-[(1r,3s)-1-(3-bromophenyl)-3-methylcyclobutyl]-1,2,4-triazole (394 mg, 1.28 mmol, 1.2 equiv) in dioxane (12 mL) was added Cs₂CO₃ (699 mg, 2.14 mmol, 2 equiv) and Xantphos (124 mg, 0.21 mmol, 0.2 equiv) and Pd(OAc)₂ (24 mg, 0.10 mmol, 0.1 equiv) in portions at room temperature under nitrogen atmosphere. The resulting mixture was stirred for 2 h at 110° C. under nitrogen atmosphere. LCMS indicated the reaction was 45% product. The mixture was allowed to cool down to room temperature. The resulting mixture was concentrated under reduced pressure. The residue was purified by silica gel column chromatography, eluted with CH₂Cl₂/MeOH (10:1) to afford 6-{2-[(tert-butyldiphenylsilyl)oxy]-1-[(3S)-3-methylpiperidin-1-yl]ethyl}-4-(methylsulfanyl)-2-{3-[(1r,3s)-3-methyl-1-(4-methyl-1,2,4-triazol-3-yl)cyclobutyl]phenyl}-3H-isoindol-1-one (270 mg, 32%) as a yellow solid. LCMS:(ES·m/z):784[M+H]⁺.

### Step 18: Synthesis of Compound 19-1

To a stirred solution of 6-{2-[(tert-butyldiphenylsilyl)oxy]-1-[(3S)-3-methylpiperidin-1-yl]ethyl}-4-(methylsulfanyl)-2-{3-[(1r,3s)-3-methyl-1-(4-methyl-1,2,4-triazol-3-yl)cyclobutyl]phenyl}-3H-isoindol-1-one (200 mg, 0.25 mmol, 1 equiv) in THE (4 mL) was added TBAF (133 mg, 0.51 mmol, 2 equiv) in portions at 0° C. The resulting mixture was stirred for 2 h at room temperature under air atmosphere. LCMS indicated the reaction was 30% product. The resulting mixture was concentrated under reduced pressure. The crude product (40 mg) was purified by Prep-HPLC with the following conditions (Column: XSelect CSH Fluoro Phenyl, 30*150 mm, 5 m; Mobile Phase A: Water (0.1% FA), Mobile Phase B: ACN; Flow rate: 60 mL/min; Gradient: 5% B to 35% B in 8 min, 35% B; Wave Length: 254; 220 nm; RT1 (min): 7.9; The collected fraction was lyophilized to afford 6-{2-hydroxy-1-[(3S)-3-methylpiperidin-1-yl]ethyl}-4-(methylsulfanyl)-2-{3-[(1r,3s)-3-methyl-1-(4-methyl-1,2,4-triazol-3-yl)cyclobutyl]phenyl}-3H-isoindol-1-one (23.7 mg, 17%) as a white solid. LCMS:(ES·m/z):546[M+H]⁺. ¹H NMR (400 MHz, DMSO-d₆) δ 8.29-8.25 (m, 2H), 8.05 (s, 1H), 7.71 (d, J=8 Hz, 1H), 7.47 (s, 2H), 7.43 (t, J=8 Hz, 1H), 7.09 (d, J=8 Hz, 1H), 3.85-3.81 (m, 1H), 3.78-3.73 (m, 1H), 3.60-3.57 (m, 1H), 3.26 (s, 3H), 2.95-2.80 (m, 3H), 2.70-2.67 (m, 1H), 2.59-2.54 (m, 6H), 2.08-1.80 (m, 1H), 1.71-1.66 (m, 1H), 1.61-1.58 (m, 2H), 1.51-1.39 (m, 3H), 1.23-1.09 (m, 3H), 0.82-0.74 (m, 4H)

### Step 19: Synthesis of Compound 21

To a stirred mixture of 6-{2-hydroxy-1-[(3S)-3-methylpiperidin-1-yl]ethyl}-4-(methylsulfanyl)-2-{3-[(1r,3s)-3-methyl-1-(4-methyl-1,2,4-triazol-3-yl)cyclobutyl]phenyl}-3H-isoindol-1-one (200 mg, 0.36 mmol, 1 equiv) and {2-[(tert-butoxycarbonyl)amino]acetamido}methyl acetate (135 mg, 0.55 mmol, 1.5 equiv) in DCM (1 mL) was added PTSA (126 mg, 0.73 mmol, 2 equiv) dropwise at 0° C. under air atmosphere. The resulting mixture was stirred for overnight at room temperature under air atmosphere. The 30% desired product was detected by LCMS. The resulting mixture was concentrated under reduced pressure. The residue was purified by reversed-phase flash chromatography with the following conditions: column, C18 silica gel; mobile phase, MeCN in Water (0.05% TFA), 0% to 60% gradient in 40 min; detector, UV 254 nm. to afford tert-butyl N-{[({2-[(3S)-3-methylpiperidin-1-yl]-2-[7-(methylsulfanyl)-3-oxo-2-{3-[(1r,3s)-3-methyl-1-(4-methyl-1,2,4-triazol-3-yl)cyclobutyl]phenyl}-1H-isoindol-5-yl]ethoxy}methyl)carbamoyl]methyl}carbamate (65 mg, 24%) as a yellow solid. LCMS: (ES·m/z): 732 [M+H]⁺.

### Step 20: Synthesis of Compound 22

To a stirred solution of tert-butyl N-{[({2-[(3S)-3-methylpiperidin-1-yl]-2-[7-(methylsulfanyl)-3-oxo-2-{3-[(1r,3s)-3-methyl-1-(4-methyl-1,2,4-triazol-3-yl)cyclobutyl]phenyl}-1H-isoindol-5-yl]ethoxy}methyl)carbamoyl]methyl}carbamate (60 mg, 0.082 mmol, 1 equiv) in DCM (1 mL) was added TFA (200 uL) dropwise at 0° C. The resulting mixture was stirred for 1 h at 0° C. LCMS indicated the reaction was completed. The resulting mixture was concentrated under reduced pressure. The residue was used to the next step directly without further purification. LCMS: (ES·m/z): 632 [M+H]⁺.

### Step 21: Synthesis of Compound 241

A mixture of (2S)-2-(2-{2-[6-(2,5-dioxopyrrol-1-yl)hexanamido]acetamido}acetamido)-3-phenylpropanoic acid (41 mg, 0.087 mmol, 1 equiv), HATU (50 mg, 0.13 mmol, 1.5 equiv) and HOBT (12 mg, 0.087 mmol, 1 equiv) in DMF (2.2 mL) was stirred for 5 min at 0° C. Then 2-amino-N-({2-[(3S)-3-methylpiperidin-1-yl]-2-[7-(methylsulfanyl)-3-oxo-2-{3-[(1r,3s)-3-methyl-1-(4-methyl-1,2,4-triazol-3-yl)cyclobutyl]phenyl}-1H-isoindol-5-yl]ethoxy}methyl)acetamide (55 mg, 0.087 mmol, 1 equiv) and DIEA (28 mg, 0.22 mmol, 2.5 equiv) was added into the above mixture at 0° C. The resulting mixture was stirred at 0° C. for another 1 h. The reaction was purified with following condition: Column: XselectCSH Prep OBD C18 Column, 30*150 mm, 5 m; Mobile Phase A: Water (0.1% FA), Mobile Phase B: ACN; Flow rate: 60 mL/min mL/min; Gradient: 20% B to 40% B in 8 min; Wave Length: 254 nm/220 nm; RT1 (min): 7.62. The collected fraction was lyophilized to afford 6-(2,5-dioxopyrrol-1-yl)-N-{[({[(1S)-1-({[({2-[(3S)-3-methylpiperidin-1-yl]-2-[7-(methylsulfanyl)-3-oxo-2-{3-[(1r,3s)-3-methyl-1-(4-methyl-1,2,4-triazol-3-yl)cyclobutyl]phenyl}-1H-isoindol-5-yl]ethoxy}methyl)carbamoyl]methyl}carbamoyl)-2-phenylethyl]carbamoyl}methyl)carbamoyl]methyl}hexanamide (2.6 mg, 2.48%) as a white solid. LCMS: (ES·m/z): 1086 [M+H]⁺. ¹H NMR (400 MHz, DMSO-d₆) δ 8.61 (br s, 1H), 8.37 (s, 1H), 8.32 (br s, 1H), 8.25-8.14 (m, 1H), 8.10-8.04 (m, 2H), 7.88 (s, 1H), 7.66-7.59 (m, 3H), 7.41 (t, J=8 Hz, 1H), 7.28-7.22 (m, 4H), 7.78-7.12 (m, 1H), 6.98 (s, 2H), 6.95 (d, J=4 Hz, 1H), 4.86 (s, 2H), 4.57-4.48 (m, 3H), 3.91-3.66 (m, 8H), 3.34-3.26 (m, 7H), 3.15-3.01 (m, 3H), 2.85-2.75 (m, 2H), 2.62 (s, 3H), 2.38-2.25 (m, 3H), 2.12-2.08 (m, 3H), 1.63-1.61 (m, 3H), 1.49-1.44 (m, 5H), 1.20-1.16 (m, 3H), 1.11 (d, J=8 Hz, 2H), 0.77 (s, 4H).

### Synthesis of Compound 242

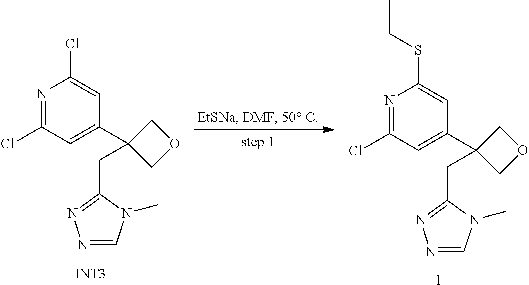

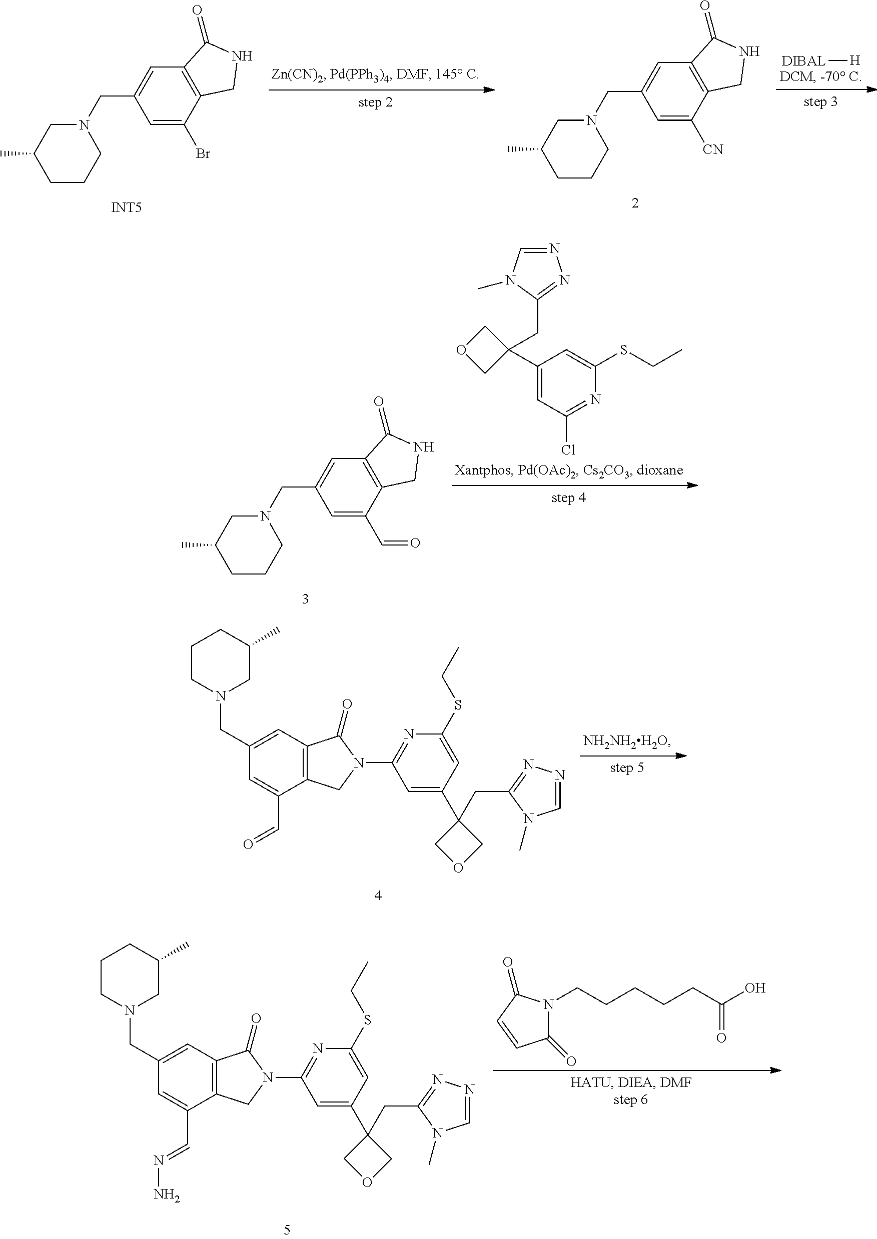

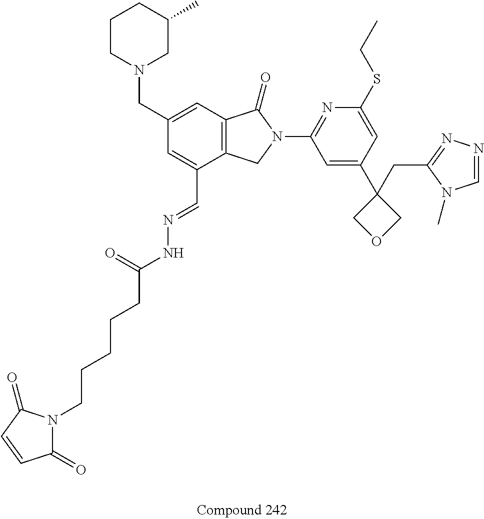 [SMILES: CCSc1cc(C2(Cc3nncn3C)COC2)cc(N2Cc3c(/C=N/NC(=O)CCCCCN4C(=O)C=CC4=O)cc(CN4CCC[C@H](C)C4)cc3C2=O)n1]

### Step 1: Synthesis of Compound 1

To a stirred solution of 2,6-dichloro-4-{3-[(4-methyl-1,2,4-triazol-3-yl)methyl]oxetan-3-yl}pyridine (1 g, 3.34 mmol, 1 equiv) in DMF (10 mL) was added (ethylsulfanyl)sodium (0.31 g, 3.67 mmol, 1.1 equiv) in portions at room temperature under nitrogen atmosphere. The resulting mixture was stirred for 2 h at 50° C. The mixture was allowed to cool down to room temperature. LCMS indicated the reaction was completed. The resulting mixture was quenched with water (50 mL) and extracted with EtOAc (3×50 mL). The combined organic layers were washed with brine (2×20 mL), dried over anhydrous Na₂SO₄. After filtration, the filtrate was concentrated under reduced pressure. The residue was purified by silica gel column chromatography, eluted with CH₂Cl₂/MeOH (10:1) to afford 2-chloro-6-(ethylsulfanyl)-4-{3-[(4-methyl-1,2,4-triazol-3-yl)methyl]oxetan-3-yl}pyridine (500 mg, 46%) as an off-white solid. LCMS: (ES·m/z): 325,327 [M+H]⁺.

### Step 2: Synthesis of Compound 2

To a stirred solution of 4-bromo-6-{[(3S)-3-methylpiperidin-1-yl]methyl}-2,3-dihydroisoindol-1-one (700 mg, 2.16 mmol, 1 equiv) in DMF (7 mL) was added Zn(CN)₂ (508 mg, 4.33 mmol, 2 equiv) and Pd(PPh₃)₄ (250 mg, 0.21 mmol, 0.1 equiv) in portions at room temperature. The resulting mixture was stirred for 1.5 h at 145° C. under nitrogen atmosphere. The mixture was allowed to cool down to room temperature. LCMS indicated the reaction was completed. The reaction mixture was purified by reversed-phase flash chromatography with the following conditions: column, C18 silica gel; mobile phase, MeCN in Water (0.05% TFA), 0% to 50% gradient in 30 min; detector, UV 254 nm. The collected fraction was concentrated to afford 6-{[(3S)-3-methylpiperidin-1-yl]methyl}-1-oxo-2,3-dihydroisoindole-4-carbonitrile (360 mg, 62%) as an off-white solid. LCMS (ESI, m/z): 270 [M+H]⁺.

### Step 3: Synthesis of Compound 3

To a stirred solution of 6-{[(3S)-3-methylpiperidin-1-yl]methyl}-1-oxo-2,3-dihydroisoindole-4-carbonitrile (360 mg, 1.33 mmol, 1 equiv) in DCM (7 mL) was added diisobutylaluminium hydride (9.5 mL, 7 equiv, 1N in DCM) dropwise at −70° C. under nitrogen atmosphere. The resulting mixture was stirred for 2 h at 0° C. under nitrogen atmosphere. LCMS indicated the reaction was completed. The reaction was quenched by the addition of MeOH (5 mL) at −20° C. The resulting mixture was concentrated under reduced pressure. The residue was purified by reversed-phase flash chromatography with the following conditions: column, C18 silica gel; mobile phase, MeCN in Water (0.05% TFA), 0% to 50% gradient in 40 min; detector, UV 254 nm. The collected fraction was concentrated to afford 6-{[(3S)-3-methylpiperidin-1-yl]methyl}-1-oxo-2,3-dihydroisoindole-4-carbaldehyde (250 mg, 68%) as a yellow solid. LCMS (ESI, m/z): 273 [M+H]⁺.

### Step 4: Synthesis of Compound 4

To a stirred mixture of 6-{[(3S)-3-methylpiperidin-1-yl]methyl}-1-oxo-2,3-dihydroisoindole-4-carbaldehyde (90 mg, 0.33 mmol, 1 equiv) and 2-chloro-6-(ethylsulfanyl)-4-{3-[(4-methyl-1,2,4-triazol-3-yl)methyl]oxetan-3-yl}pyridine (97 mg, 0.29 mmol, 0.9 equiv) in dioxane (1 mL) was added Cs₂CO₃ (215 mg, 0.66 mmol, 2 equiv) and Xantphos (38 mg, 0.06 mmol, 0.2 equiv) and Pd(OAc)₂ (7 mg, 0.03 mmol, 0.1 equiv) in portions at room temperature under nitrogen atmosphere. The resulting mixture was stirred for 2 h at 110° C. under nitrogen atmosphere. The mixture was allowed to cool down to room temperature. LCMS indicated the reaction was completed. The resulting mixture was concentrated under reduced pressure. The residue was purified by silica gel column chromatography, eluted with CH₂Cl₂/MeOH (10:1) to afford 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-1-oxo-3H-isoindole-4-carbaldehyde (70 mg, 14%) as a white solid. LCMS:(ES·m/z):561[M+1]⁺.

### Step 5: Synthesis of Compound 5

To a stirred solution of 2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-1-oxo-3H-isoindole-4-carbaldehyde (70 mg, 0.12 mmol, 1 equiv) in EtOH (1 mL) was added hydrazine hydrate (12 mg, 0.25 mmol, 2 equiv) dropwise at 0° C. under nitrogen atmosphere. The resulting mixture was stirred for 1 h at room temperature under nitrogen atmosphere. LCMS indicated the reaction was completed. The resulting mixture was concentrated under reduced pressure. The resulting mixture was used in the next step directly without further purification. LCMS (ESI, m/z): 575 [M+H]⁺.

### Step 6: Synthesis of Compound 242

To a stirred solution of 2-[6-(ethylsulfanyl)-4-{3-[(4-methyl-1,2,4-triazol-3-yl)methyl]oxetan-3-yl}pyridin-2-yl]-4-methanehydrazonoyl-6-{[(3S)-3-methylpiperidin-1-yl]methyl}-3H-isoindol-1-one (50 mg, 0.08 mmol, 1 equiv) in DMF (1 mL) was added HATU (44 mg, 0.12 mmol, 1.5 equiv), N-maleoyl-6-aminohexanoic acid (22 mg, 0.10 mmol, 1.2 equiv) and DIEA (34 mg, 0.26 mmol, 3 equiv) dropwise at 0° C. under nitrogen atmosphere. The resulting mixture was stirred for 2 h at room temperature under nitrogen atmosphere. LCMS indicated the reaction was ˜40% product. The reaction mixture was purified by Prep-HPLC with the following conditions (Column: XBridge Prep Phenyl OBD C18 Column, 30*150 mm, 5 m; Mobile Phase A: Water (10 mmol/L NH₄HCO₃), Mobile Phase B: ACN; Flow rate: 60 mL/min mL/min; Gradient: 18% B to 48% B in 20 min; Wave Length: 254 nm/220 nm; RT1 (min): 18.1). The collected fraction was lyophilized to afford 6-(2,5-dioxopyrrol-1-yl)-N′-[(1E)-{2-[6-(ethylsulfanyl)-4-{3-[(4-methyl-1,2,4-triazol-3-yl)methyl]oxetan-3-yl}pyridin-2-yl]-6-{[(3S)-3-methylpiperidin-1-yl]methyl}-1-oxo-3H-isoindol-4-yl}methylidene]hexanehydrazide (7.7 mg, 11%) as a white solid. LCMS (ESI, m/z): 768 [M+H]⁺. ¹H NMR (400 MHz, DMSO-d₆) δ 11.51-11.42 (m, 1H), 8.26-8.17 (m, 2H), 8.08-8.02 (m, 1H), 7.80-7.72 (m, 2H), 7.00-6.82 (m, 3H), 5.24-5.18 (m, 2H), 4.93-4.90 (m, 2H), 4.85-4.84 (m, 2H), 3.58-3.55 (m, 4H), 3.40-3.35 (m, 2H), 3.29-3.26 (m, 3H), 3.08-3.07 (m, 1H), 2.72-2.66 (m, 3H), 2.25-2.20 (m, 1H), 1.95-1.89 (m, 1H), 1.67-1.49 (m, 9H), 1.34-1.24 (m, 6H), 0.90-0.82 (m, 4H).

### Example 6: Preparation of Conjugates

### Preparation of Antibody ‘A’-Compound 205 Conjugate

A mixture of 2 mg of Antibody ‘A’, 16.9 mg/mL in 20 mM histidine, 250 mM sucrose pH 6.5, was treated with 40 equivalents of TCEP and incubated at 37° C. for 1 h to fully reduce the interchain disulfide bonds. The reduced antibody was purified into 50 mM EPPS, 5 mM EDTA pH 7.0 using NAP5 desalting columns. Conjugation was effected by diluting the antibody and adding 12 molar equivalents of Compound 205 as a stock solution in DMA such to give a reaction mixture consisting of 2 mg/mL reduced antibody+12 eq. Compound 205 in 50 mM EPPS, 5 mM EDTA pH 7.0+15% DMA. The reaction was incubated for 1 h at ambient temperature. Conjugate was purified into 20 mM succinate, 8% sucrose, 0.01% Tween-20 pH 5.5 using NAP10 columns. The conjugate (1.5 mL) was collected into a 50K MWCO Amicon Ultra 4 centrifugal concentrator, diluted with 1 mL 20 mM succinate, 8% sucrose, 0.01% Tween-20 pH 5.5 and concentrated to 0.5-1.0 mL. This process was repeated twice for a total of three cycles. Conjugate was found to 8 drug/antibody by reducing LC-MS, 97.9% monomer by SEC, and no detectable free drug by mixed-mode HPLC on a HISEP column.

### Preparation of Antibody ‘E’-Compound 205 Conjugate

A mixture of 1.5 mg of Antibody ‘E’, 7.5 mg/mL in 25 mM sodium borate, 25 mM sodium chloride, 1 mM EDTA pH 8.0 was treated with 20 equivalents of TCEP and incubated at 37° C. for 4 h to fully reduce the interchain disulfide bonds. The reduced antibody was purified into 50 mM EPPS, 5 mM EDTA pH 7.0 a Zeba 40K desalting columns. Conjugation was effected by diluting the antibody and adding 12 molar equivalents of Compound 205 as a stock solution in DMA such to give a reaction mixture consisting of 4.5 mg/mL reduced antibody+12 eq. Compound 205 in 50 mM EPPS, 5 mM EDTA pH 7.0+10% DMA. The reaction was incubated for 1 h at ambient temperature, then quenched with 0.01 volumes of N-acetylcysteine. Conjugate was purified into 20 mM succinate, 8% sucrose, 0.01% Tween-20 pH 5.5 using a Zeba 40K desalting column, the concentrated using a 50K MWCO Amicon Ultra 4 centrifugal concentrator. Conjugate was found to 8 drug/antibody by reducing LC-MS, 100% monomer by SEC, and no detectable free drug by mixed-mode HPLC on a HISEP column.

### Preparation of Antibody ‘A’-Compound 214 Conjugate

A mixture of Antibody ‘E’, 16.9 mg/mL in 20 mM histidine, 250 mM sucrose pH 6.5, was treated with 10 eq. TCEP to fully reduce the interchain disulfides. The reduced antibody was purified into 50 mM EPPS, 5 mM EDTA pH 7.0 using Zeba 40K desalting columns. Conjugation was effected by diluting the antibody and adding 12 molar equivalents of Compound 214 as a stock solution in DMA such to give a reaction mixture consisting of 4.5 mg/mL reduced antibody+12 eq. Compound 214 in 50 mM EPPS, 5 mM EDTA pH 7.0+10% DMA. The reaction was incubated for 1 h at ambient temperature, then quenched with 0.01 volumes of N-acetylcysteine. Conjugate was purified into 20 mM succinate, 8% sucrose, 0.01% Tween-20 pH 5.5 by two cycles of gel filtration using Zeba 40K desalting columns. Conjugate was found to have 6.5 drug/antibody by reducing LC-MS, 90.9% monomer by SEC, and no detectable free drug by mixed-mode HPLC on a HISEP column.

### Preparation of Antibody “E’-Compound 214 Conjugate

A mixture of Antibody ‘A’, 8 mg/mL in 25 mM borate, 25 mM sodium chloride, 1 mM EDTA pH 8.0 was treated with 20 eq. TCEP to fully reduce the interchain disulfide bonds. The reduced antibody was purified into 50 mM EPPS, 5 mM EDTA pH 7.0 using NAP desalting columns. Reduced antibody was stored at −80 C and thawed for conjugation. Conjugation was effected by diluting the antibody and adding 12 molar equivalents of Compound 214 as a stock solution in DMA such to give a reaction mixture consisting of 3.6 mg/mL reduced antibody+12 eq. Compound 214 in 50 mM EPPS, 5 mM EDTA pH 7.0+10% DMA. The reaction was incubated for 2 h at ambient temperature, then quenched with 0.01 volumes of N-acetylcysteine. Conjugates were purified by two rounds of gel filtration using NAP columns. In the first round, columns were equilibrated and conjugates were eluted with 20 mM succinate, 8% sucrose, 0.01% Tween-20 pH 5.5+10% DMA. In the second round, columns were equilibrated and conjugates were eluted with 20 mM succinate, 8% sucrose, 0.01% Tween-20 pH 5.5. Finally, conjugates were concentrated using Amicon centrifugal concentrators. Conjugate was found to have 8 drug/antibody by reducing LC-MS, 97.4% monomer by SEC, and no detectable free drug by mixed-mode HPLC on a HISEP column.

### Preparation of Antibody ‘E’-Compound 212 (Lysine conjugation)

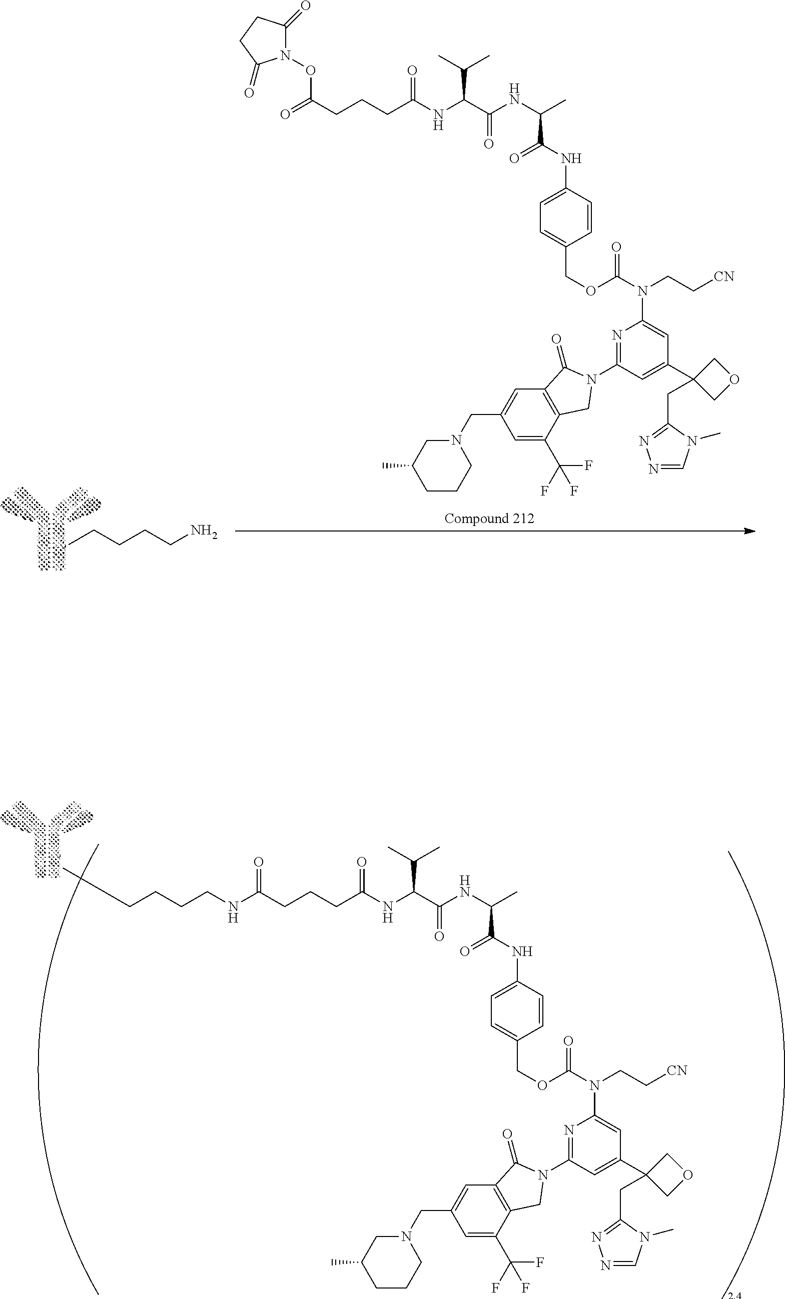

To a solution of Antibody ‘E’ in 50 mM EPPS pH 8.0 was added 10 molar equivalents of Compound 212 as a stock solution in DMA to give a reaction mixture consisting of 5 mg/mL antibody+10 eq. Compound 212 in 50 mM EPPS pH 8.0+10% DMA. The reaction was incubated at ambient temperature for 3 hours. Conjugate was purified into 20 mM succinate, 8% sucrose, 0.01% Tween-20 pH 5.5 formulation buffer using Zeba 40K desalting columns. Conjugate was found to have an average of 2.4 drug/antibody by native SEC-MS and >99% monomer by SEC.

### Preparation of IgG-Compound 500 (TLR7/8 Agonist (T785) Conjugate (Prophetic Example)

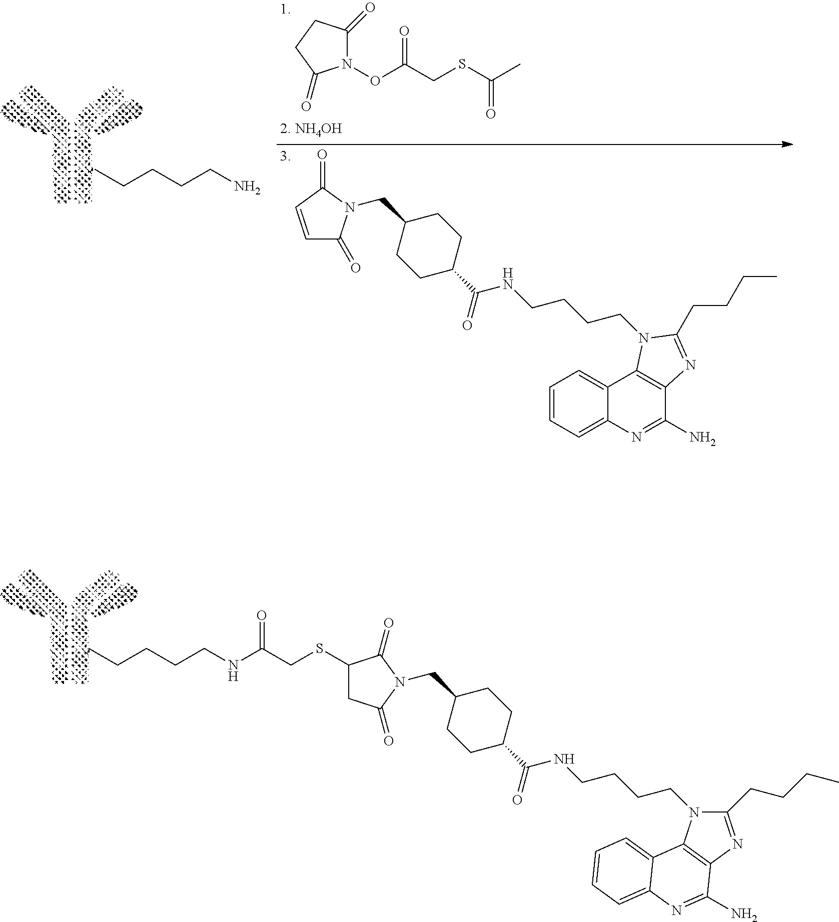

To a solution of 5-10 mg/mL immune cell targeting antibody in 50 mM EPPS pH 8.0 is added SATA as a stock solution in DMA such that the final DMA concentration is 10% (v/v). The SATA is titrated to target 2.5 moles of conjugated linker per mole of antibody. The reaction is incubated for 1 h at ambient temperature. To deprotect the thiols, 0.1 volumes of 500 mM hydroxylamine, 25 mM EDTA in 50 mM EPPS pH 8.0 is added and the reaction incubated for 2 h at ambient temperature. The thiolated antibody intermediate is purified into 50 mM EPPS, 5 mM EDTA pH 7.0 using NAP desalting columns. To the thiolated antibody intermediate is added 3.75 molar equivalents of Compound 500 (MCC-T785, described in Ackerman, S. E., Pearson, C. I., Gregorio, J. D. et al. Nat Cancer 2, 18-33 (2021). https://doi.org/10.1038/s43018-020-00136-x) such that the final concentration of DMA is 10%. The reaction is incubated for 2 h at ambient temperature. The conjugate is purified into 20 mM succinate, 8% sucrose, 0.01% Tween-20 pH 5.5 using NAP desalting columns.

### Preparation of Antibody “I”-Compound 221-DAR 8 Conjugate (Endogenous cysteine conjugation)

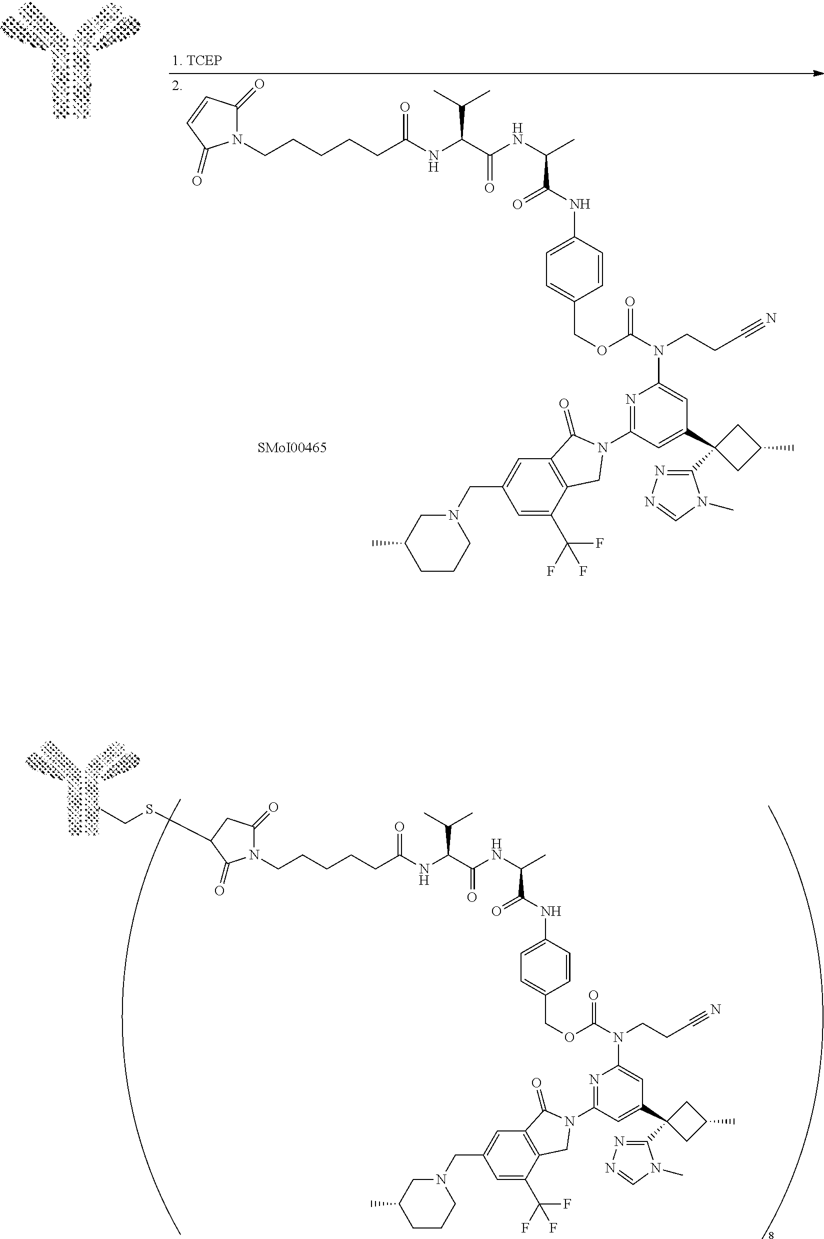

A mixture of 2 mg of Antibody ‘I’, 13.38 mg/mL in 20 mM Histidine, 250 mM Sucrose, pH 6.5 were treated with excess TCEP(aq) (2 uL of 0.5 mM) and incubated at 37° C. for 1 h to fully reduce the interchain disulfide bonds. The reduced antibody was purified into 50 mM EPPS, 5 mM EDTA pH 7.0 using NAP-5 desalting column (GE Healthcare) according to the manufacturer's instructions. Conjugation was effected by diluting the antibody with conjugation buffer ‘50 mM EPPS, 5 mM EDTA pH 7.0’ and DMA, then adding 12 molar equivalents of maleimide-functionalized linker payload ‘Compound 221’ as a 10 mM stock solution in DMA such to give a reaction mixture consisting of 4.44 mg/mL reduced antibody, 12 eq. of Compound 221 in ‘50 mM EPPS, 5 mM EDTA, 15% DMA’. The reaction was incubated for 1 h at ambient temperature. Conjugate was purified into 20 mM succinate, 8% sucrose, 0.01% Tween-20 pH 5.5 using NAP-5 desalting column (GE Healthcare) according to the manufacturer's instructions, then concentrated using a 50K MWCO Amicon Ultra 4 centrifugal concentrator, diluted with 1 mL 20 mM succinate, 8% sucrose, 0.01% Tween-20 pH 5.5, and concentrated to ˜0.5 mL. This process was repeated twice for a total of three cycles. The concentrate was then filtered through 0.22 um PVDF syringe filters. Antibody “I”-Compound 221 conjugate were obtained (1.64 mgs, 2.44 mg/mL), 74% yield, 8 drug/antibody ratio by reducing LC-MS, 98.4% monomer ratio by SEC, and no detectable free drug by mixed-mode HPLC on a HISEP column.

### Preparation of Antibody “I”-Compound 220 Conjugate (Lysine conjugation)

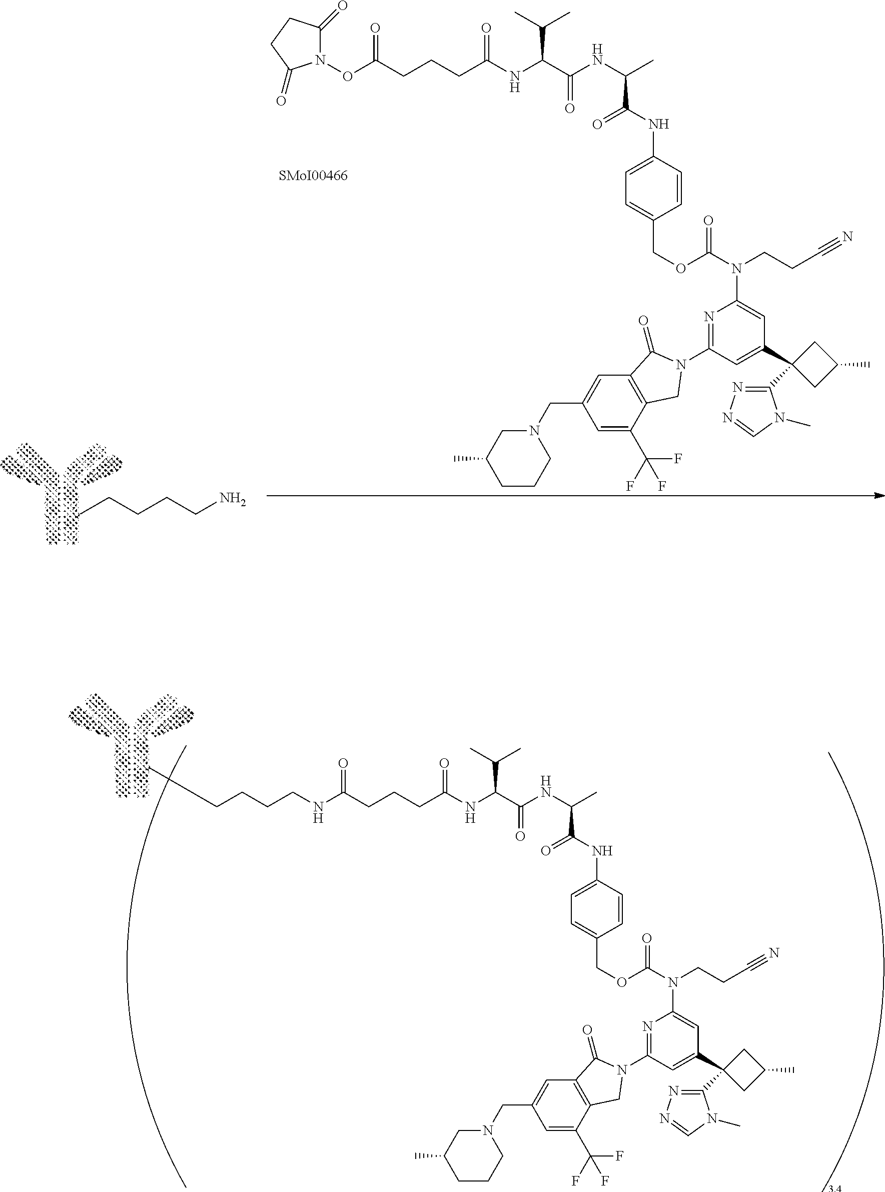

To a solution of 1 mg Antibody ‘I’ in 50 mM EPPS, 5 mM EDTA, pH 7.0, 10 molar equivalents of Compound 220 was added as a 10 mM stock solution in DMA to give a reaction mixture consisting of 3 mg/mL antibody+10 eq. Compound 220 in 50 mM EPPS, pH 7.0, 5 mM EDTA, 20% DMA. The reaction was incubated at ambient temperature for 5 hours. Conjugate was purified into formulation buffer '20 mM succinate, 8% sucrose, 0.01% Tween-20 pH 5.5 ‘using NAP5 columns (GE Healthcare) according to the manufacturer's instructions, then concentrated using a 50K MWCO Amicon Ultra 4 centrifugal concentrator, diluted with 1 mL 20 mM succinate, 8% sucrose, 0.01% Tween-20 pH 5.5 and concentrated to ˜0.5 mL. This process was repeated twice for a total of three cycles. Then the concentrate was filtered through 0.22 um PVDF syringe filters. 0.83 mgs of Antibody ‘I’-Compound 220 conjugate were obtained as 1.92 mg/mL, 83% yield, 3.4 drug/antibody ratio by native SEC-MS, 100% monomer ratio by SEC, and 2.5% free drug by mixed-mode HPLC on a HISEP column.

### Example 7: Biological Activity of Conjugates

### PD1/PD-L1 Blockade Assay Measuring Activity of AntiPD1-Cbl-b Inhibitor Conjugates

Jurkat NFAT/PD1 reporter cells (Promega, J1252) were seeded into white 96 well plates (ThermoFisher, 136101) at 3×10⁴ cells/well in 50 μl of assay media (RPMI1640 supplemented with 2% FBS). Serial dilutions of pembrolizumab (control) or pembrolizumab antibody drug conjugates to various small molecules (test articles) were prepared in assay media and added to the cells in duplicates. CHO/PD-L1 activator cells (Promega, J1252) were harvested using Trypsin/EDTA. The CHO/PD-L1 activator cells were then washed with RPMI1640 with 10% FBS, resuspended in assay media and added at 2×10⁴ cells/well to the Jurkat NFAT/PD1 cells. The plates were then incubated at 37° C. with 5% CO₂ for 24 hours. After incubating, BioGlo reagent (Promega, G7940) was added to the cells per manufacturer's protocol and the resulting luminescence was measured using a SpectraMax M5 plate reader (Molecular Devices).

Results are reported as the fold change over background of the luminescent signals from test article treated wells compared to the baseline luminescence from corresponding control wells. Compounds were sorted into ranges as follows: values between 3.51 and 5.49: range “X;” values between 2.5 and 3.5: range “A;” values between 2.0 and 2.49: range “B;” values between 1.5 and 1.99: range “C;” and values between 1.1 and 1.49: range “D.”

TABLE 7
PDI/PDLI Blockade Activity of Conjugates

| Conjugate | 70 nM | 7 nM |
|---|---|---|
| Antibody ‘E’-Compound 214 (DAR 8) | A | A |
| Antibody ‘E’-Compound 201 (DAR 8) | D | D |
| Antibody ‘E’-Compound 206 (DAR 8) | B | B |
| Antibody ‘E’-Compound 204 (DAR 5.6) | — | — |
| Antibody ‘E’-Compound 207 (DAR 8) | A | B |
| Antibody ‘E’-Compound 209 (DAR 8) | D | D |
| Antibody ‘E’-Compound 203 (DAR 8) | C | D |
| Antibody ‘E’-Compound 212 (DAR 2.51) | B | C |
| Antibody ‘E’-Compound 201 (DAR 7.8) | B | C |
| Antibody ‘E’-Compound 211 (DAR 8.0) | C | D |

TABLE 7A
PDI/PDLI Blockade Activity of Conjugates

Score

| | DAR | 70 nM | 7 nM |
|---|---|---|---|
| Antibody ‘I’- Compound 215 | 3.6 | X | X |
| Antibody ‘I’- Compound 220 | 4.4 | X | X |
| Antibody ‘E’- Compound 221 | 8 | X | X |
| Antibody ‘E’- Compound 220 | 2.8 | X | X |
| Antibody ‘I’- Compound 221 | 8 | X | A |
| Antibody ‘E’- Compound 215 | 2 | X | X |
| Antibody ‘I’- Compound 216 | 8 | X | X |
| Antibody ‘I’- Compound 232 | 7.5 | D | D |
| Antibody ‘E’- Compound 211 | 8 | B | C |
| Antibody ‘E’- Compound 223 | 3.6 | B | C |
| Antibody ‘E’- Compound 212 | 3.3 | X | B |
| Antibody ‘I’- Compound 218 | 7.8 | X | C |
| Antibody ‘I’- Compound 217 | 8 | D | D |
| Antibody ‘I’- Compound 233 | 7.9 | X | C |
| Antibody ‘I’- Compound 230 | 8 | X | C |
| Antibody ‘I’- Compound 228 | 7.2 | D | D |
| Antibody ‘I’- Compound 235 | 8 | C | D |
| Antibody ‘I’- Compound 221 | 8 | B | B |
| Antibody ‘I’- Compound 222 | 8 | X | A |
| Antibody ‘I’- Compound 222 | 4 | B | B |
| Antibody ‘I’- Compound 207 | 8 | B | C |
| Antibody ‘E’- Compound 207 | 8 | B | C |
| Antibody ‘E’- Compound 222 | 8 | B | B |
| Antibody ‘E’- Compound 221 | 8 | B | B |

### T Cell Activation by Anti-CD25 CD7-Cbl-b Inhibitor Conjugates

Human primary T cells were isolated from commercially available frozen peripheral blood mononuclear cells (PBMC, HemaCare Corporation) or from fresh LeukoPaks (Research Blood Components). T cells were isolated through depletion of non-T cells using negative selection kit according to the manufacturer's protocol (Miltenyi Biotec, Human Pan T Cell Isolation Kit, 130-096-535). Isolated T cells were stimulated with CD3/CD28 Dynabeads (ThermoFisher, 111.31D) for 48 hours with 2 μL of pre-washed bead per 2×10⁵ T cells. Beads were removed from the cell culture with a magnet (Miltenyi Biotec) and activated T cells were washed twice using XVivol5 media (Lonza, 04-418Q). 5×10⁴ cells per well were added to 96 well plates coated with anti-CD3 (OKT3) antibody. To coat the plates for T cell capture, anti-CD3 antibody was diluted in PBS at 10 μg/mL and 100 μl added to each well. Plates were incubated at 4° C. overnight or at 37° C. for 2 hours and washed once with PBS before use. Serial dilutions of each conjugate were added in duplicates to the T cells to assess their effect on T cell activation. Plates were incubated with serial dilutions of non-binding antibody drug conjugate (control) or anti-CD25 or anti-CD7 antibody drug conjugates (test articles) or unconjugated antibody for 48 hours at 37° C. with 5% CO₂, and supernatants were subsequently analyzed for levels of the activation induced secreted cytokines IL-2 or IFN-y (BD Biosciences CBA Flex Beads, 558269, 558270) by flow cytometry (ThermoFisher Attune NxT).

Results are reported as fold change over background where background is the IL-2 or IFN-y concentration produced by T cells treated with non-binding control conjugates or corresponding unconjugated antibody. Normalized T cell IL-2 and IFN-y responses were sorted into ranges as follows: for IFN-y, values between 1.00 and 1.49: range “D;” 1.50-1.99: range “C;” 2.00-2.49: range “B;” and values between 2.5 and 3.0: range “A.” For IL-2, values between 2.00 and 4.00: range “D;” 4.00-4.99: range “C;” 5.00-5.99: range “B;” and values between 6.00 and 8.00: range “A”.

TABLE 8
T Cell Activation Calculated as Fold Change
Over Background (Unconjugated Antibody)

| | IFN-y | | IL-2 | |
|---|---|---|---|---|
| Conjugate | 100 nM | 10 nM | 100 nM | 10 nM |
| Antibody ‘A’-Compound 203 | B | B | B | B |
| Antibody ‘A’--Compound 204 | B | B | B | B |
| Antibody ‘A’--Compound 205 | A | A | A | A |
| Antibody ‘A’--Compound 214 | A | B | B | C |
| Antibody ‘A’--Compound 207 | A | C | A | C |
| Antibody ‘A’--Compound 209 | C | C | D | D |
| Antibody ‘A’--Compound 201 | B | B | C | B |
| Antibody ‘H’--Compound 201 | C | C | D | D |
| Antibody ‘H’--Compound 205 | D | D | C | D |

TABLE 9
T Cell Activation Calculated as Fold Change Over
Background (Non-Binding Antibody Conjugate)

| Conjugate | IFN-y, 10 nM | IL-2, 10 nM |
|---|---|---|
| Antibody ‘A’--Compound 203 | C | C |
| Antibody ‘A’--Compound 204 | B | C |
| Antibody ‘A’--Compound 205 | B | B |
| Antibody ‘A’--Compound 214 | B | D |
| Antibody ‘A’--Compound 207 | C | D |
| Antibody ‘A’--Compound 209 | C | D |
| Antibody ‘A’--Compound 201 | B | C |
| Antibody ‘H’--Compound 201 | D | D |
| Antibody ‘H’--Compound 205 | D | D |

### Mixed Lymphocyte Reaction

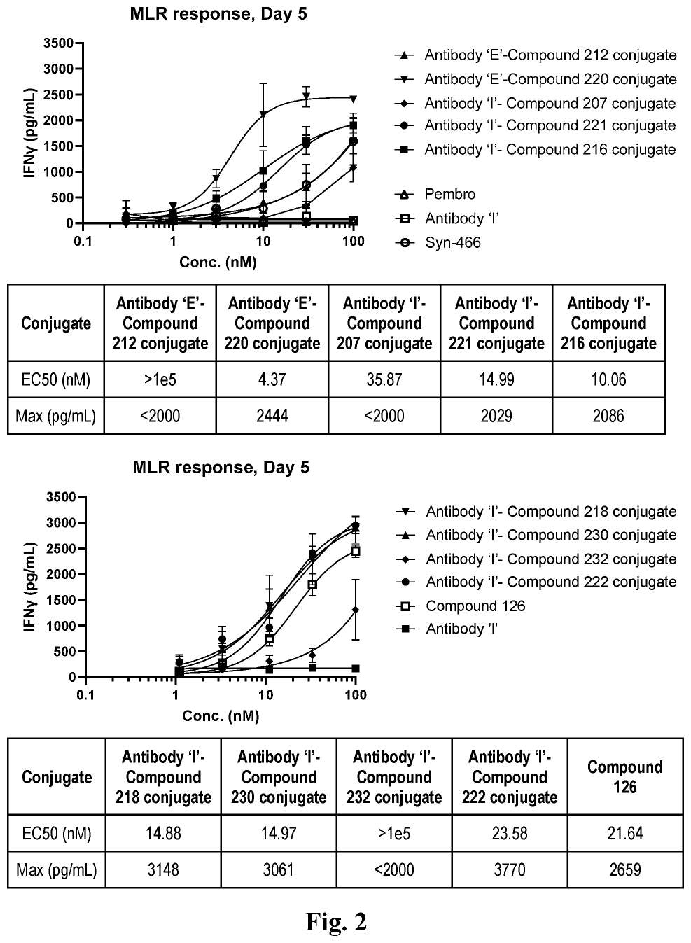

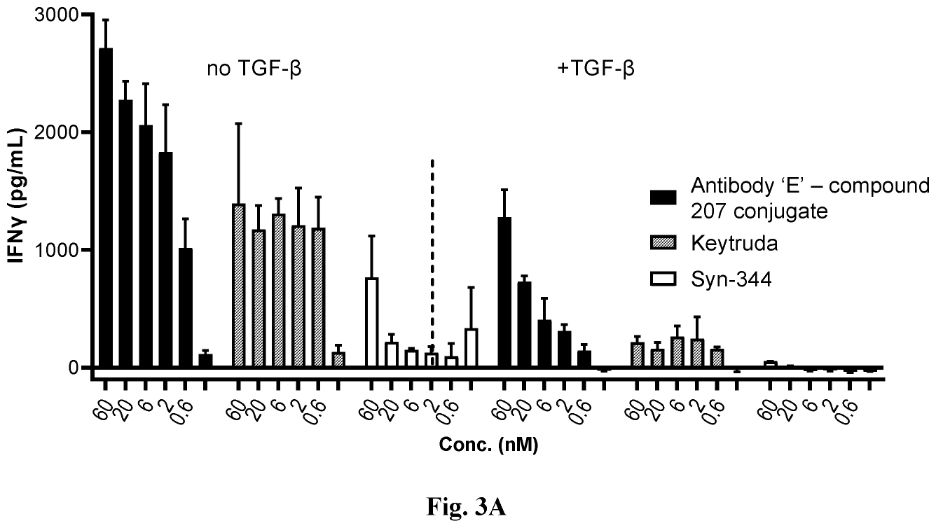

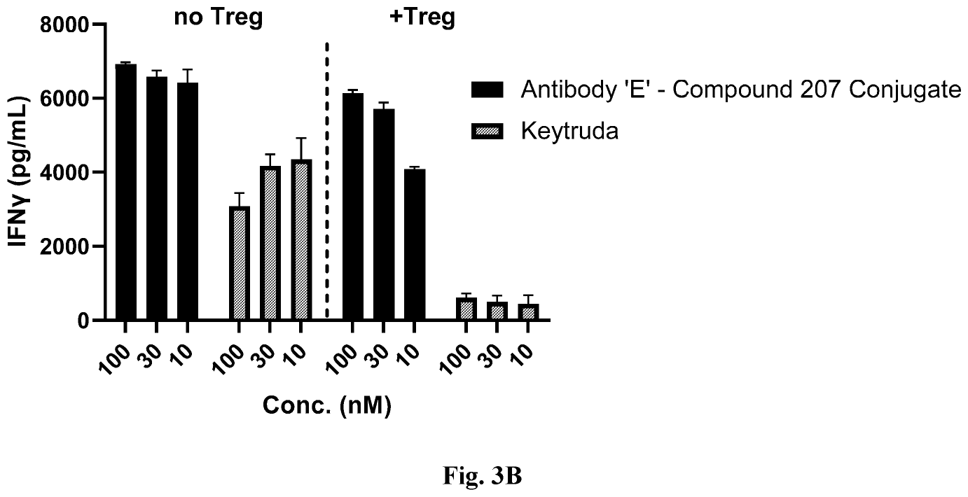

Human primary monocytes were isolated from cryopreserved PBMCs purchased commercially (HemaCare Corporation) by positive selection of CD14⁺ cells according to the manufacturer's protocol (Miltenyi Biotec, CD14 MicroBeads, 130-050-201). Monocytes were differentiated into mature dendritic cells (stimulator cells) by culturing in GM-CSF and IL-4 for 7 days according to the manufacturer's protocol for human dendritic cell differentiation kit (R&D Systems, CDK004). Stimulator cells were harvested and seeded in 96-well flat-bottomed tissue culture plates at 1×10⁴ cells per well. Human primary CD4+ T cells from allogeneic donor were isolated by negative selection according to manufacturer's protocol (Miltenyi Biotec, CD4+ T cell Isolation Kit, 130-096-533). CD4+ T cells (responder cells) were co-cultured with stimulator cells at 10:1 ratio for 5 days, at 37° C., in XVivo 15 media (Lonza, 04-418Q). Treatment with serially-diluted conjugates, unconjugated antibody, or non-binding antibody drug conjugate were added at the start of the co-culture. To test the effect of TGF-β suppression on treatment, recombinant human TGF-β1 (R&D Systems, 240-B) was added at 2 ng/mL. To assess whether conjugates mitigate the suppressive effect of Treg cells on T cell activation, human natural Treg cells (iQ Biosciences, IQB-Hul-nTr-1) were added at the start of co-culture at the CD4+ T cell:Treg ratio of 10:3. At the end of 5 days, supernatants were collected and induction of secreted cytokines IL-2 or IFN-γ were quantitated (BD Biosciences CBA Flex Beads, 558269, 558270) by flow cytometry (ThermoFisher Attune NxT). The results are shown in FIGS. 2, 3A, and 3B. The results demonstrate immune cell-targeting antibody conjugated to Cbl-b inhibitor is an approach that enables potent enhancement of primary T cell responses in a dose-dependent manner, and that the effect is resistant to T cell suppression by both TGF-β1 and Treg cells.

### Example 8: T Cell Activation by Small Molecule Inhibitors of Cbl-b

Cryopreserved human peripheral blood pan-T cells (StemCell, 70024) were defrosted and resuspended in Immunocult-XF T cell Expansion Medium (StemCell, 10981). Cells were plated at 8×10⁵ cells per well in 24 well plates, in presence or absence of pre-coated anti-CD3 (OKT3) antibody (ThermoFisher, 16-0037-85). Pre-coating of plates was done by diluting anti-CD3 antibody to 10 μg/mL in PBS and adding 1 ml to each well. Plates were incubated at 4° C. overnight or at 37° C. for 2 hours and washed once with PBS before use. Dilution of each small molecule cbl-b inhibitor was added. Untreated cell was used as a control. Plates were incubated for 48 hours at 37° C. with 5% CO₂. T cells were fixed and permeabilized with Intracellular Fixation & Permeabilization Buffer Set (ThermoFisher, 88-8824), and continuously stained with one of PE-conjugated antibody listed in Table 10. Stained T cells were then analyzed with by flow cytometry (ThermoFisher Attune NxT). The percent of positive cells of each treated group was normalized with untreated cell and sorted into bins as follows: fold over background values from 10 to 14.9: range “A;” values from 5.0 to 9.9: range “B;” values from 2.0 to 4.9: range “C;” and values from 0.1 to 1.9: range “D.” Only for Notch1, geometric mean was used. The results are reported in Table 11.

TABLE 10
Antibodies for Detection of Biomarkers for T Cell Activiation

| Antibody | Company | Catalog number |
|---|---|---|
| PE-conjugated anti-pZAP70 | Cell Signaling | 14791 |
| PE-conjugated anti-pPLCg1 | Cell Signaling | 14461 |
| PE-conjugated anti-pPLCg2 | Thermo | 12-9866-42 |
| PE-conjugated anti-pHS1 | Cell Signaling | 11880S |
| PE-conjugated anti-pLYN | Cell Signaling | 94361 |
| PE-conjugated anti-pCD3zeta | Thermo | 12-2478-42 |
| PE-conjugated anti-Notch1 | R&D systems | FAB5317P-100 |

TABLE 11
T Cell Activiation

| | | Notch 1 | pHS1 | pZAP70 | pLYN | pPLCg1 | pPLCg2 | pCD3zeta |
|---|---|---|---|---|---|---|---|---|
| −CD3 | Compound 147 | D | C | B | D | D | C | C |
| | (10 μM) | | | | | | | |
| | Compound 126 | D | B | C | C | C | B | C |
| | (10 μM) | | | | | | | |
| | Compound 192 | D | A | C | C | B | A | C |
| | (10 μM) | | | | | | | |
| +CD3 | Compound 147 | C | C | C | D | D | D | D |
| | (10 μM) | | | | | | | |
| | Compound 126 | C | C | C | D | C | D | D |
| | (10 μM) | | | | | | | |
| | Compound 192 | C | C | C | D | C | D | D |
| | (10 μM) | | | | | | | |

### Example 9: In Vitro TIL Activation

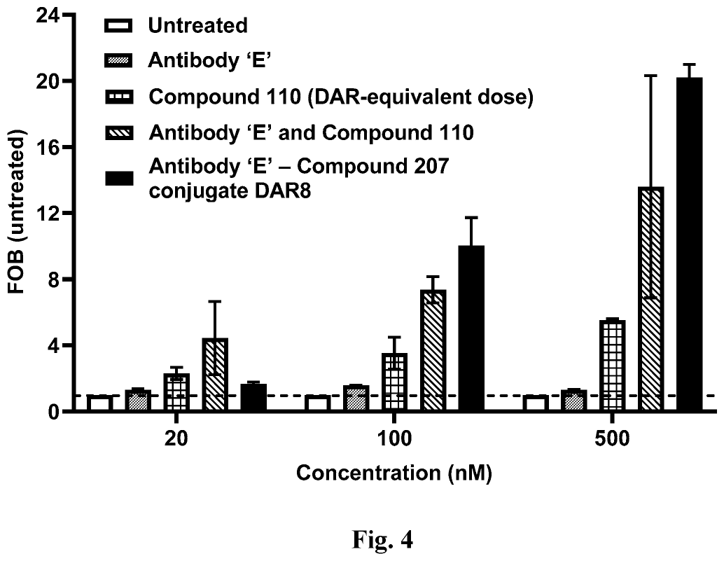

Mature dendritic cells (DCs) were generated from cryopreserved human monocytes (StemCell, 70034) after thaw, by culturing in ImmunoCult™-ACF Dendritic Cell Medium (StemCell, 10986) containing ImmunoCult™-ACF Dendritic Cell Differentiation Supplement (StemCell, 10988) at a hundredth volume of media. Media containing the differentiation supplement was refreshed every 2-3 days for a week, and ImmunoCult™ Dendritic Cell Maturation Supplement (StemCell, 10989) was added into the media at a hundredth volume to mature the DCs over 3 days. Frozen human melanoma bulk dissociated tumor cells (DTCs, Discovery Life Sciences) were thawed and co-seeded in Immunocult-XF T cell Expension Medium (StemCell, 10981) with the mature DCs in a 96-well plate, 5×10⁴ DTCs and 1×10⁴ DCs in 100 μl per well. The allogeneic mature DCs serve as stimulator cells for tumor-infiltrating lymphocytes (TILs) in the DTCs. An equivalent volume of test article in various dilutions was added on top in duplicate. An untreated condition was used as a normalization control. Plates were then incubated for 72 hours at 37° C. with 5% CO₂. The supernatants were subsequently analyzed for levels of IFN-y (R&D systems, DY285B), as a measure of T cell activation. The level of IFNγ detected in each condition was normalized to that of the untreated condition. The results are shown in FIG. 4.

### Example 10: In Vivo Activity

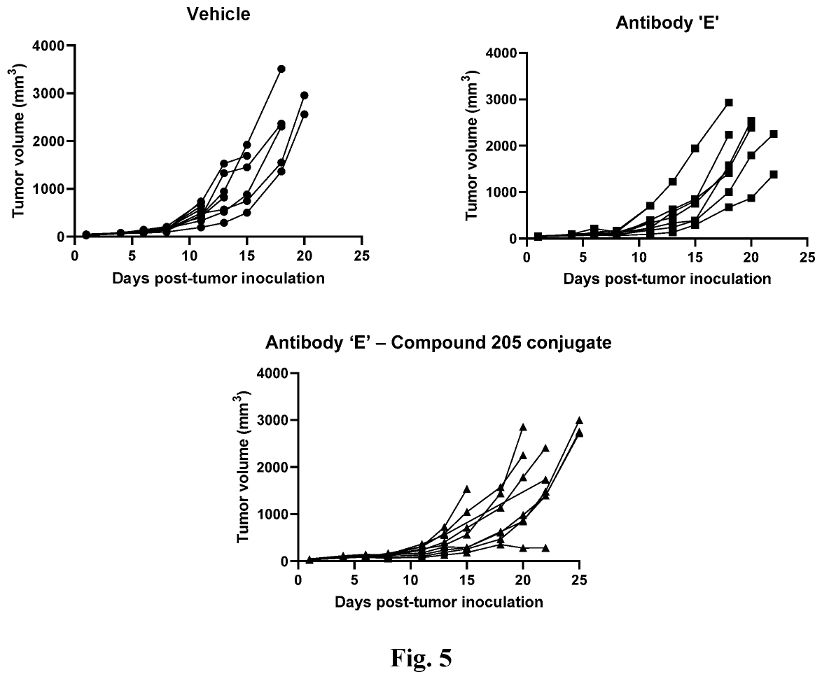

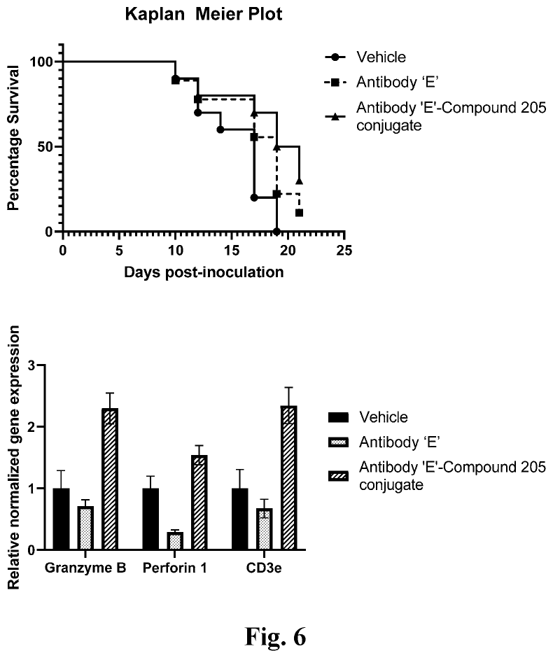

B-hPD-1 plus mice (Jiangsu Biocytogen Co. Ltd., China) used were homozygous, genetically engineered C₅₇BL/6 mice whose ectodomain region of endogenous mouse Pdcd1 was replaced by human PD-1. All experimental mice were housed and used in accordance with animal care guidelines from the Institutional Animal Care and Use Committee. The B16F10 murine melanoma cell line was maintained at Biocytogen Boston Corp, in Dulbecco's Modified Eagle's medium (DMEM) supplemented with 10% Fetal Bovine Serum. B16F10 cells growing in exponential growth phase were harvested and inoculated subcutaneously, at 0.5×10⁶ tumor cells in 0.1 mL serum-free medium in the right flank. Mice were randomized on day 4, and test articles were administered intravenously at 10 mg/kg. Mice were dosed again on days 7 and 10. Tumor measurements were taken every 3-4 days with calipers and expressed in tumor volume (V), where V=W²×L/2 (W=width measured along the short axis of the tumor; and L=length measured along the long axis of the tumor). Mice were sacrificed 32 days after initial tumor inoculation, or at humane endpoint, and tumors were snap-frozen for gene expression evaluation by relative qPCR. Briefly, total RNA was isolated using Quick-RNA Microprep Kit (Zymo Research; catalog #R1051). cDNA synthesis and qPCR reaction was set up using LunaScript RT SuperMix Kit and Luna Universal qPCR Master Mix respectively. Taqman probes used for the qPCR reactions are listed in Table 12. The results are shown in FIGS. 5 and 6.

TABLE 12
Probes for Gene Detection

| Species | Gene Name (symbol) | Taqman Probe number |
|---|---|---|
| Mouse | glyceraldehyde-3-phosphate | Mm99999915_g1 |
| | dehydrogenase (Gapdh) | |
| Mouse | granzyme B (Gzmb) | Mm00442837_m1 |
| Mouse | perforin 1 (Prf1) | Mm00812512_m1 |
| Mouse | Cd3 (Cd3e) | Mm01179194_m1 |

### Example 11: Exhausted T Cell Activation by Anti-PD-1-Cbl-b Inhibitor Conjugates

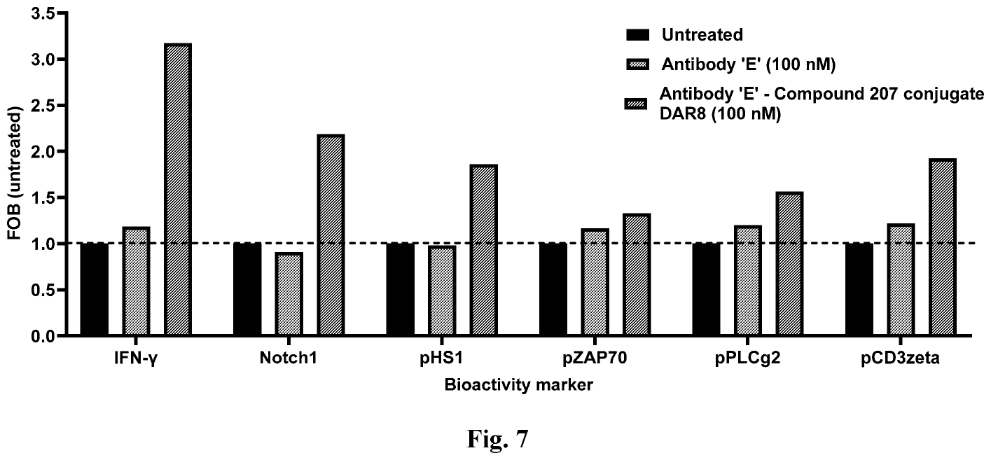

CD4+ T cells were isolated from cryopreserved human peripheral blood T cells (StemCell, 70024) using MagCellect Human Naive CD4+ T Cell Isolation Kit (R&D systems, MAGH115). CD4+ T cells were resuspended at 5×10⁵ cells per ml in Immunocult-XF T cell Expension Medium (StemCell, 10981) in the presence of Dynabeads human T activator CD3/CD28 (ThermoFisher, 11132D). To prepare exhausted T cells, CD4+ T cells were re-stimulated with the activator every 2-3 days for 2 weeks. The exhausted CD4+ T cells were co-cultured with CHO/PD-L1 activator cells (Promega, J1252) at 2:1 ratio. Prior to start, CHO/PD-L1 activator cells were harvested using Trypsin/EDTA and plated at 5×10⁴ cells per well in RPMI1640 with 10% FBS into 24-well plate. The plates were incubated at 37° C. with 5% CO₂ for 24 hours before use. The supernatants were discarded and washed with PBS, follow by adding 500 μl of exhausted CD4+ T cells in serum-free RPMI1640. An equivalent volume of each tested article was added. Untreated condition was used as a control. Plates were then incubated for 48 hours at 37° C. with 5% CO₂. The supernatants were subsequently analyzed for levels of IFN-7 (R&D systems, DY285B). The cells were fixed and permeabilized with Intracellular Fixation & Permeabilization Buffer Set (ThermoFisher, 88-8824), and continuously stained with APC-conjugated anti-CD3 (ThermoFisher, 17-0038-42) and with one of the PE-conjugated antibodies listed in Table 10. Stained T cells were then analyzed with by flow cytometry (ThermoFisher Attune NxT). Results are reported in FIG. 7 as the fold change over background (FOB) of the untreated condition. An increase in expression of not only IFNγ, but also biomarkers associated with T cell activation, was evident following treatment with anti-PD-1-Cbl-b inhibitor conjugates.

### Example 12: CEF MHC Class 1-Restricted Antigen Recall Assay

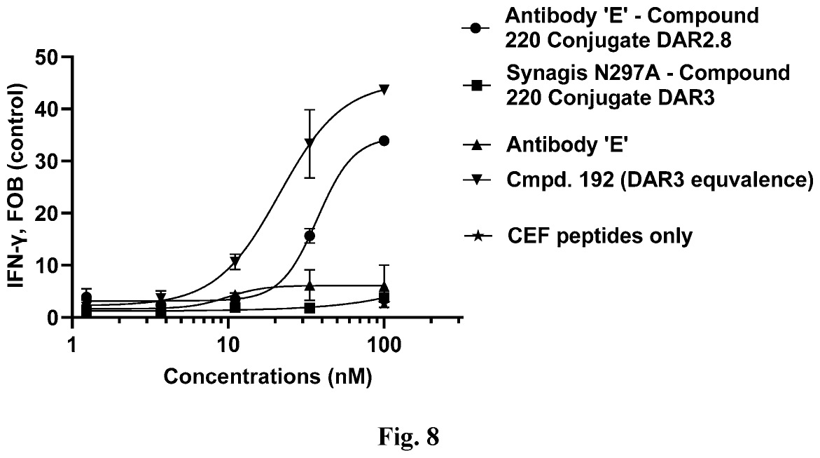

Human PBMCs (StemCell, ST70025) were seeded at 1×10⁵ cells per well in round-bottomed 96-well plate with DC differentiating medium (StemCell, 10981 and 10986), at 200 μL per well. Following a 24-hour incubation, 100 μL of culture medium was discarded. PepTivator CEF MHC Class I (Miltenyi, 130-098-426) was diluted in Immunocult-XF T cell Expansion Medium (StemCell, 10981) and added to the cells at 100 μL per well, according to manufacturer's recommended concentration. The plates were incubated for 72 hours, and the removal of spent medium and addition of fresh CEF-containing medium was repeated. The incubation was continued for 3 more days. 100 μL of culture medium was removed, and 100 μL of diluted testing article in Immunocult-XF T cell Expansion Medium was added to each well. The cells were treated for 4 days, and supernatants were subsequently analyzed for levels of IFN-7 (R&D systems, DY285B). The results from this assay (FIG. 8) revealed that anti-PD-1-Cbl-b inhibitor conjugates enhanced and sustained antigen recall response of T cells stimulated 2 rounds with CMV, EBV, and influenza peptide pool.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

1. A compound of formula (I): 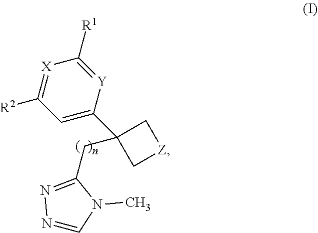
or a pharmaceutically acceptable salt thereof, wherein:
n is 0, 1, or 2;
X and Y are each independently CH or N;
Z is selected from CH(CH₃), O, and SO₂; or
Z is selected from CH(CH₃), NH, N(CH₃), O, and SO₂;
R¹ is selected from hydrogen, —CN, —NHR^{z}, —R^{a}, —NR^{a}R^{b}, —OR^{a}, —NHC(O)R^{a}, —NHC(S)R^{a}, —NHC(O)NHR^{a}, —NHC(S)NHR^{a}, —SR^{a}, C₃-C₆cycloalkyl, and a 3- to 6-membered heterocyclyl ring;
wherein:
R^{z} is selected from 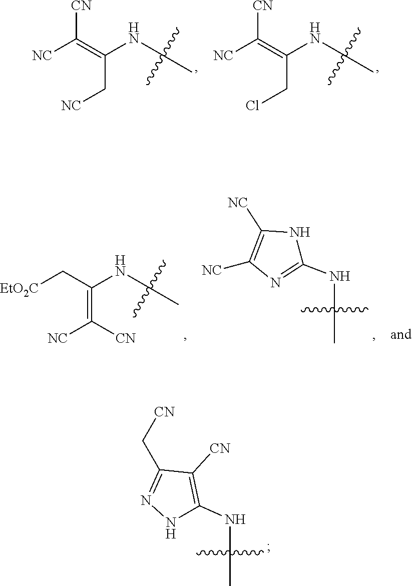 [SMILES: *NC(CC#N)=C(C#N)C#N ; *NC(CCl)=C(C#N)C#N ; *NC(CC(=O)OCC)=C(C#N)C#N ; *Nc1nc(C#N)c(C#N)[nH]1 ; *Nc1[nH]nc(CC#N)c1C#N]
R^{a} and R^{b} are independently selected from hydrogen, C₂-C₆alkenyl, C₁-C₆alkyl, amido(C₁-C₆alkyl), amino(C₁-C₆alkyl), azido(C₁-C₆alkyl), C₂-C₆alkynyl, carboxy(C₁-C₆alkyl), cyano(C₁-C₆alkyl), C₃-C₆cycloalkyl optionally substituted with a cyano group, dimethylamino(C₁-C₆alkyl), a 3- to 6-membered heterocyclyl ring, 3-6-membered heterocyclyl(C₁-C₃alkyl), hydroxy(C₁-C₆alkyl), methoxy(C₁-C₆alkyl), methylamino(C₁-C₆alkyl), NR^{c}R^{d}(C₁-C₆alkyl), HS(C₁-C₆alkyl), and CH₃S(C₁-C₆alkyl), wherein R^{c} and R^{d} are independently selected from hydrogen, C₂alkenylcarbonyl, and methyl; or,
R^{a} and R^{b}, together with the nitrogen atom to which they are attached, form a five- or six-membered ring optionally containing one additional nitrogen atom, wherein the ring is optionally substituted with one group selected from R^{a}, —C(O)R^{a}, —SO₂R^{a}, azido, and cyano;
wherein each C₃-C₆cycloalkyl, each 3- to 6-membered heterocyclyl ring, and the heterocyclyl part of the 3- to 6-membered heterocyclyl(C₁-C₃alkyl) ring are optionally substituted with one, two, or three groups independently selected from C₁-C₃alkyl, C₂alkynyl, amido, azido, carboxy, cyano, dimethylamino, hydroxy, methoxy, methylamino, HS—, and CH₃S—; and
R² is selected from 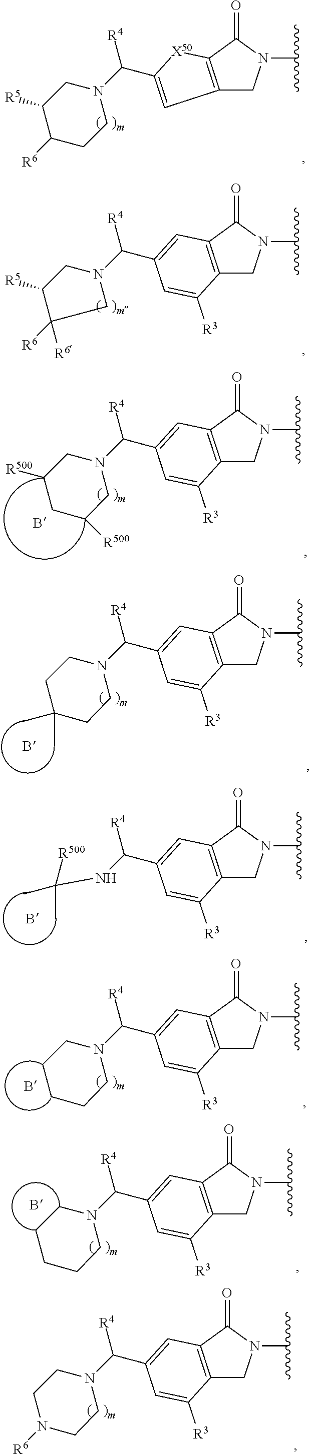 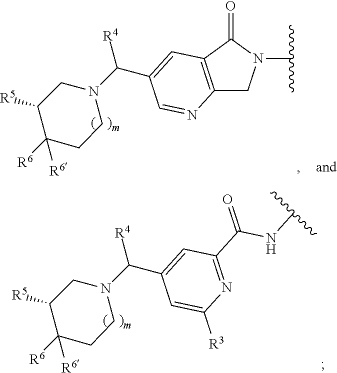
wherein
m is 0, 1, 2, or 3;
m″ is 0, 1, 2, 3, or 4;
B′ is a 3-7 membered saturated or unsaturated ring optionally containing one or two heteroatoms independently selected from nitrogen, oxygen, and sulfur; wherein the ring is optionally substituted with one or two substituents independently selected from —OH, —CH₂SH, CH₂SCH₃, —CH₂OH, —CH₂NH₂, and —CH₂NHCH₃;
each R⁵⁰⁰ is independently selected from hydrogen, C₁-C₆alkyl, halo, —OH, and —CH₂OH; and
X⁵⁰ is selected from O, NH, NCH₃, and S;
R³ is selected from hydrogen, acetyl, amino, C₁-C₆alkylamino, C₁-C₆alkylaminomethyl, C₁-C₆alkylcarbonyl, aminoC₁-C₆alkyl, aminocarbonyl, aminomethyl, carboxy, cyano, C₃cycloalkyl, formyl, hydroxy, hydroxyC₁-C₆alkyl, methoxy, oxazolyl, —SH, —SCH₃, —SOCH₃, —SO₂CH₃, —SO(═NH)CH₃, tetrazolyl, thiazolyl, and trifluoromethyl, wherein the C₃cycloalkyl is optionally substituted with a hydroxy group;
R⁴ is selected from hydrogen, methyl, —CH₂OH, —CH₂SH, and —CH₂SCH₃;
R⁵ is selected from hydrogen, hydroxy, —CH₂SH, —CH₂SCH₃, and methyl;
optionally provided that when R⁵ is hydroxy or methyl, and R⁴ is hydrogen, then R¹ is other than C₃-C₆cycloalkyl, a 3- to 6-membered heterocyclyl ring, hydroxy, hydroxy(C₁-C₆alkyl), —OR^{a} wherein R^{a} is C₁-C₆alkyl, a 3- to 6-membered heterocyclyl ring, or hydroxy(C₁-C₆alkyl); or —NR^{a}R^{b}, wherein R^{a} and R^{b} are independently selected from the group consisting of hydrogen, C₁-C₆alkyl, C₃-C₆cycloalkyl, hydroxy(C₁-C₆alkyl), a 3- to 6-membered heterocyclyl ring, or wherein R^{a} and R^{b}, together with the nitrogen atom to which they are attached, form a five- or six-membered ring optionally containing one additional nitrogen atom, wherein the ring is optionally substituted with one group selected from C₁-C₆alkyl or hydroxy(C₁-C₆alkyl); and
R⁶ and R^{6′} are independently selected from hydrogen, cyclopropyl, —CH₂OH, —CH₂SH, —CH₂SCH₃, and —CH₂R²⁰⁰, wherein R²⁰⁰ is a 3-7 membered saturated or unsaturated ring optionally containing one or two heteroatoms independently selected from nitrogen, oxygen, and sulfur.

2. A compound of claim 1, or a pharmaceutically acceptable salt thereof, wherein:
X is N;
Y is CH; and
R² is

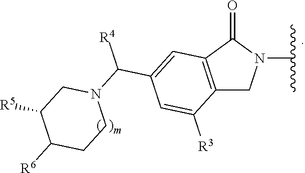

3. A compound of claim 1, or a pharmaceutically acceptable salt thereof, wherein Z is O or wherein Z is CH(CH₃).

4. (canceled)

5. A compound of claim 1, or a pharmaceutically acceptable salt thereof, wherein:
R³ is trifluoromethyl;
R⁴ and R⁶ are hydrogen; and
R⁵ is methyl.

6. A compound of claim 1, or a pharmaceutically acceptable salt thereof, wherein R¹ is selected from —NR^{a}R^{b}, —NHC(O)R^{a}, —NHC(S)NHR^{a}, —SR^{a}, —SCH₂NH(CH₂CN, and —NH(CH₂)₂N₃.

7-9. (canceled)

10. A compound of formula (IA-1): 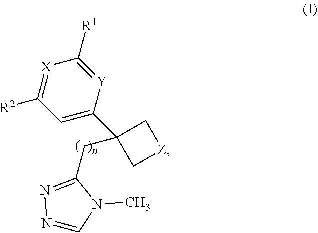
or a pharmaceutically acceptable salt thereof, wherein:
n is 0, 1, or 2;
X and Y are each independently CH or N;
Z is selected from CH(CH₃), O, and SO₂;
R¹ is selected from hydrogen, —CN, —NHR^{z}, —R^{a}, —NR^{a}R^{b}, —OR^{a}, —NHC(O)R^{a}, —NHC(S)R^{a}, —NHC(O)NHR^{a}, —NHC(S)NHR^{a}, —SR^{a}, C₃-C₆cycloalkyl, and a 3- to 6-membered heterocyclyl ring;
wherein:
R^{z} is selected from 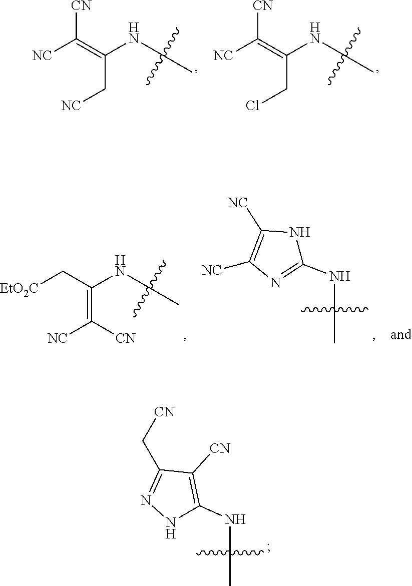 [SMILES: *NC(CC#N)=C(C#N)C#N ; *NC(CCl)=C(C#N)C#N ; *NC(CC(=O)OCC)=C(C#N)C#N ; *Nc1nc(C#N)c(C#N)[nH]1 ; *Nc1[nH]nc(CC#N)c1C#N]
R^{a} and R^{b} are independently selected from hydrogen, C₂-C₆alkenyl, C₁-C₆alkyl, amido(C₁-C₆alkyl), amino(C₁-C₆alkyl), azido(C₁-C₆alkyl), C₂-C₆alkynyl, carboxy(C₁-C₆alkyl), cyano(C₁-C₆alkyl), C₃-C₆cycloalkyl, dimethylamino(C₁-C₆alkyl), a 3- to 6-membered heterocyclyl ring, 3-6-membered heterocyclyl(C₁-C₃alkyl), hydroxy(C₁-C₆alkyl), methoxy(C₁-C₆alkyl), methylamino(C₁-C₆alkyl), NR^{c}R^{d}(C₁-C₆alkyl), HS(C₁-C₆alkyl), and CH₃S(C₁-C₆alkyl), wherein R^{c} and R^{d} are independently selected from hydrogen, C₂alkenylcarbonyl, and methyl; or,
R^{a} and R^{b}, together with the nitrogen atom to which they are attached, form a five- or six-membered ring optionally containing one additional nitrogen atom, wherein the ring is optionally substituted with one group selected from R^{a}, —C(O)R^{a}, —SO₂R^{a}, azido, and cyano;
wherein each C₃-C₆cycloalkyl, each 3- to 6-membered heterocyclyl ring, and the heterocyclyl part of the 3- to 6-membered heterocyclyl(C₁-C₃alkyl) ring are optionally substituted with one, two, or three groups independently selected from C₁-C₃alkyl, C₂alkynyl, amido, azido, carboxy, cyano, dimethylamino, hydroxy, methoxy, methylamino, HS—, and CH₃S—; and
R² is 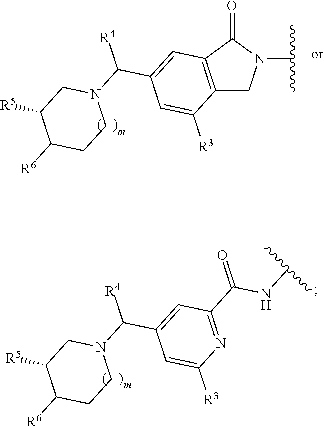
wherein
m is 0, 1, 2, or 3;
R³ is selected from hydrogen, acetyl, amino, C₁-C₆alkylamino, C₁-C₆alkylaminomethyl, aminoC₁-C₆alkyl, aminocarbonyl, aminomethyl, carboxy, cyano, C₃cycloalkyl, formyl, hydroxy, hydroxyC₁-C₆alkyl, methoxy, oxazolyl, —SH, —SCH₃, tetrazolyl, thiazolyl, and trifluoromethyl, wherein the C₃cycloalkyl is optionally substituted with a hydroxy group;
R⁴ and R⁶ are independently selected from hydrogen, —CH₂SH, and —CH₂SCH₃; and
R⁵ is selected from hydroxy, —CH₂SH, —CH₂SCH₃, and methyl;
optionally provided that when R⁵ is hydroxy or methyl, and R⁴ is hydrogen, then R¹ is other than C₃-C₆cycloalkyl, a 3- to 6-membered heterocyclyl ring, hydroxy, hydroxy(C₁-C₆alkyl), —OR^{a} wherein R^{a} is C₁-C₆alkyl, a 3- to 6-membered heterocyclyl ring, or hydroxy(C₁-C₆alkyl); or —NR^{a}R^{b}, wherein R^{a} and R^{b} are independently selected from the group consisting of hydrogen, C₁-C₆alkyl, C₃-C₆cycloalkyl, hydroxy(C₁-C₆alkyl), a 3- to 6-membered heterocyclyl ring, or wherein R^{a} and R^{b}, together with the nitrogen atom to which they are attached, form a five- or six-membered ring optionally containing one additional nitrogen atom, wherein the ring is optionally substituted with one group selected from C₁-C₆alkyl or hydroxy(C₁-C₆alkyl),

11. A compound of claim 1, selected from 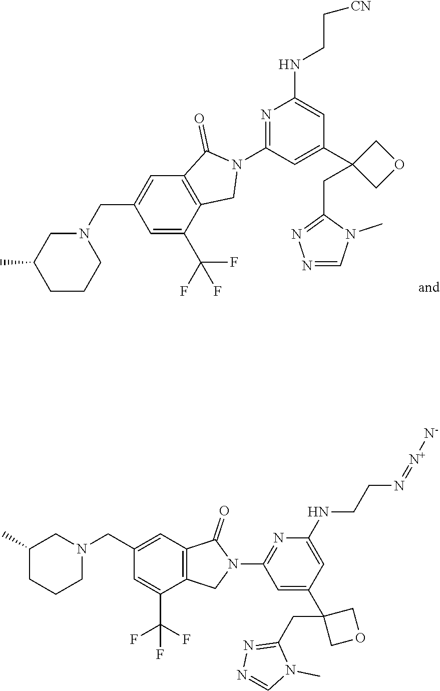 [SMILES: C[C@H]1CCCN(Cc2cc3c(c(C(F)(F)F)c2)CN(c2cc(C4(Cc5nncn5C)COC4)cc(NCCC#N)n2)C3=O)C1 ; C[C@H]1CCCN(Cc2cc3c(c(C(F)(F)F)c2)CN(c2cc(C4(Cc5nncn5C)COC4)cc(NCCN=[NH+][NH-])n2)C3=O)C1]
or a pharmaceutically acceptable salt thereof.

12. A compound selected from: 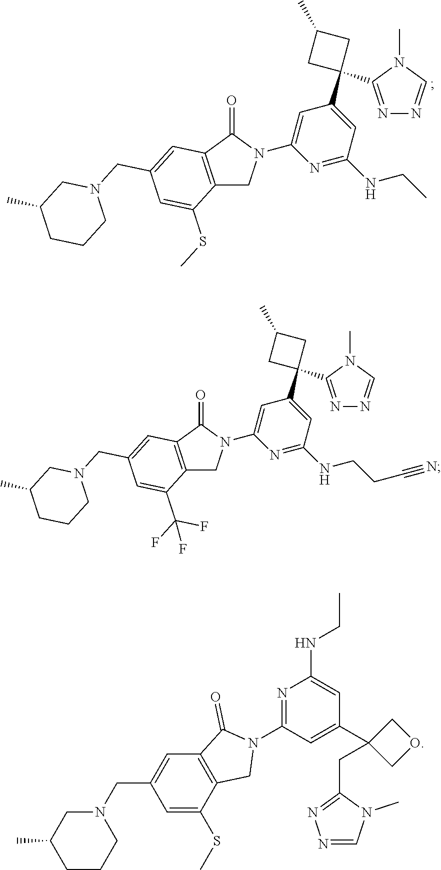
and

13-14. (canceled)

15. A conjugate, or a pharmaceutically acceptable salt thereof, comprising a binding moiety that is capable of specifically binding to a target on the surface of an effector T cell and a payload that is capable of activating an effector T cell, wherein the binding moiety is directly attached to the payload or is attached to the payload through a linker.

16-18. (canceled)

19. A conjugate, or a pharmaceutically acceptable salt thereof, comprising a binding moiety that is capable of specifically binding to PD1 and (ala payload that is capable of activating an effector T cell, wherein the binding moiety is directly attached to the payload or is attached to the payload through a linker or (b) a payload that is an inhibitor of Casitas B-lineage lymphoma proto-oncogene b (Cbl-b).

20. (canceled)

21. The conjugate of claim 15, or a pharmaceutically acceptable salt thereof, wherein the conjugate has formula (I):
Bm-ₐ (I),
wherein:
a is an integer from 1 to 50;
P is the payload;
L is a linker; and
Bm is the binding moiety.

22. The conjugate of claim 15, or a pharmaceutically acceptable salt thereof, wherein the payload is (a) an inhibitor of Casitas B-lineage lymphoma proto-oncogene b (Cbl-b); (b) an agonist of toll-like receptor 7 (TLR-7) and/or toll-like receptor 8 (TLR-8); (c) an inhibitor of hematopoietic progenitor kinase 1 (HPK-1); (d) an inhibitor of STING, phosphoinositide-3-kinase gamma (PI3Ky), CXCR4, CCR5, or a mitogen-activated protein kinase (MAPK) pathway protein, or (e) an agonist of stimulator of interferon genes (STING.

23. (canceled)

24. The conjugate of claim 22, or a pharmaceutically acceptable salt thereof, wherein inhibitor of Cbl-b is 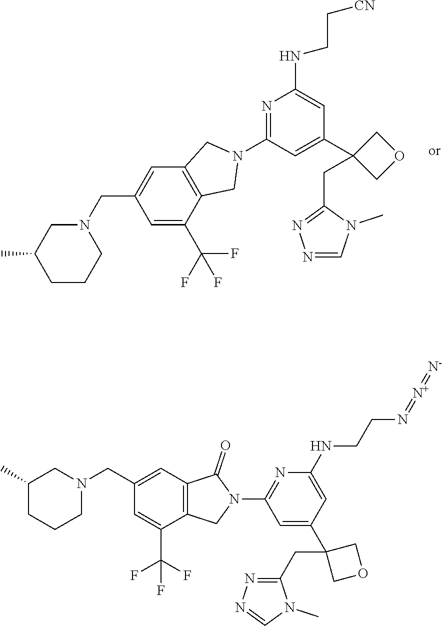 [SMILES: C[C@H]1CCCN(Cc2cc3c(c(C(F)(F)F)c2)CN(c2cc(C4(Cc5nncn5C)COC4)cc(NCCC#N)n2)C3)C1 ; C[C@H]1CCCN(Cc2cc3c(c(C(F)(F)F)c2)CN(c2cc(C4(Cc5nncn5C)COC4)cc(NCCN=[N+]=[N-])n2)C3=O)C1]
Compound 146, Compound 147, Compound 148, or NX-1607.

25-31. (canceled)

32. The conjugate of claim 15, or a pharmaceutically acceptable salt thereof, wherein the payload is a small molecule or a peptide.

33-54. (canceled)

55. A conjugate of claim 21, selected from: 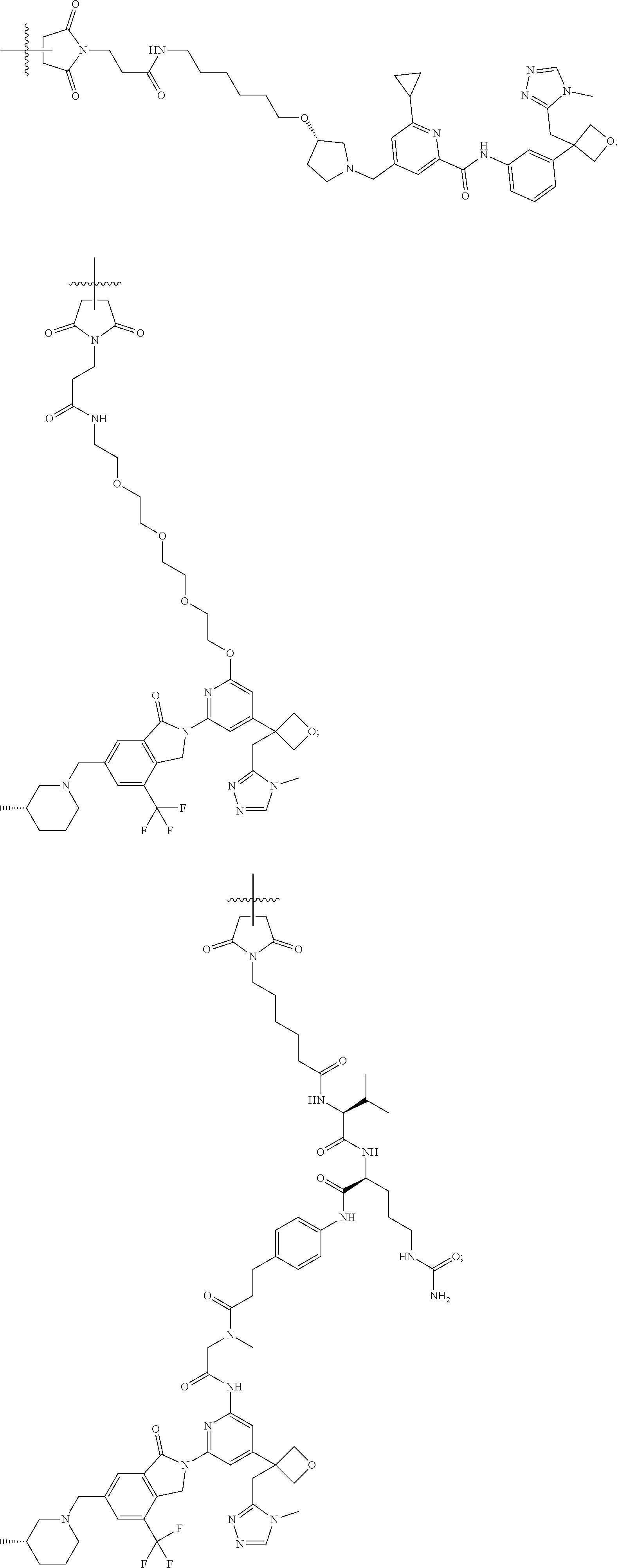 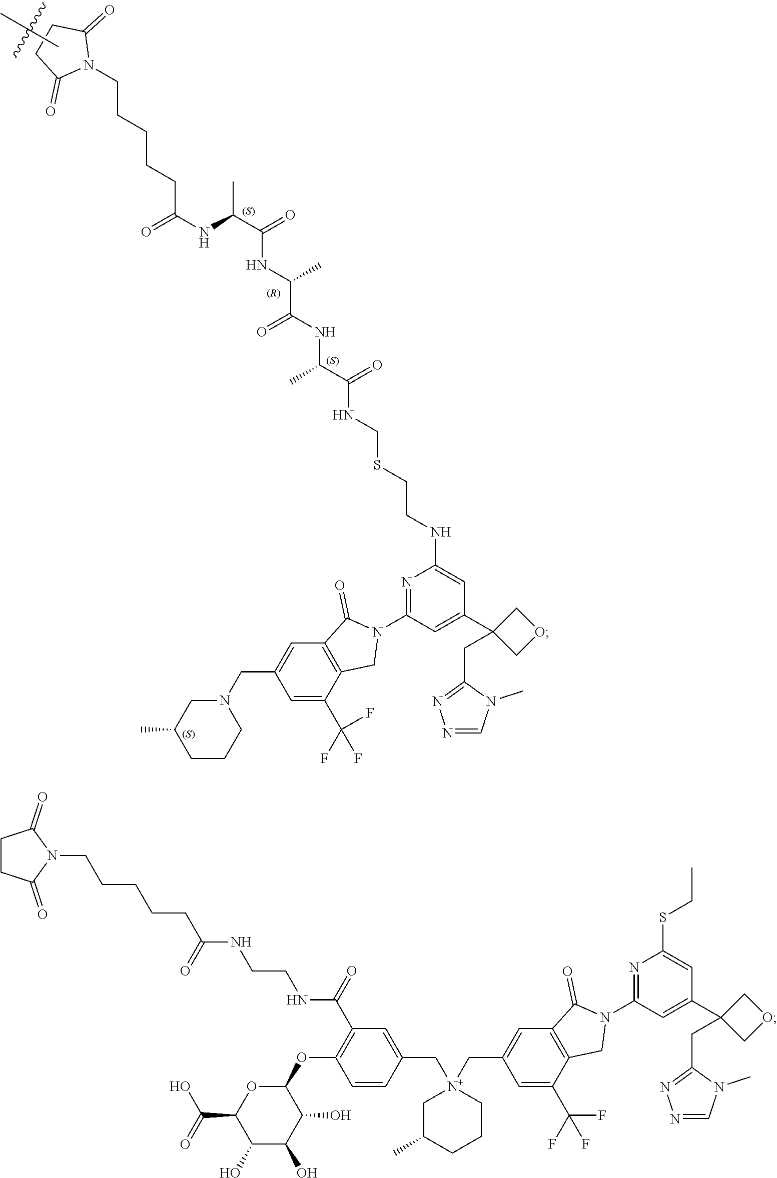 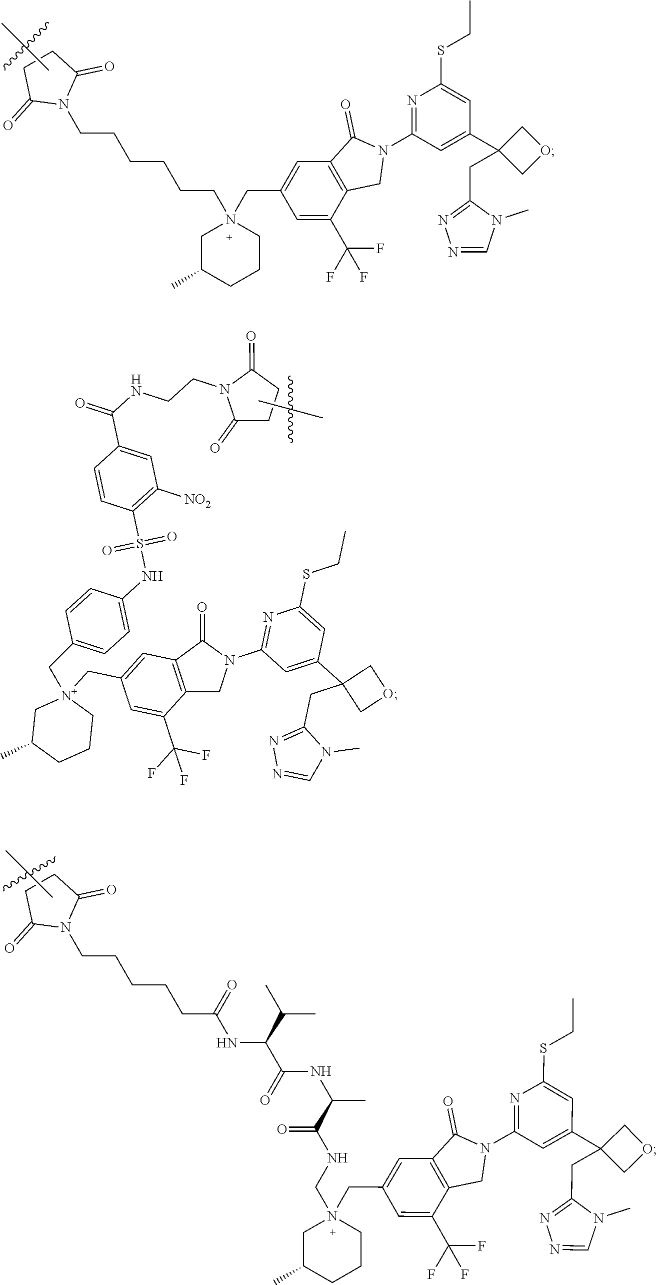 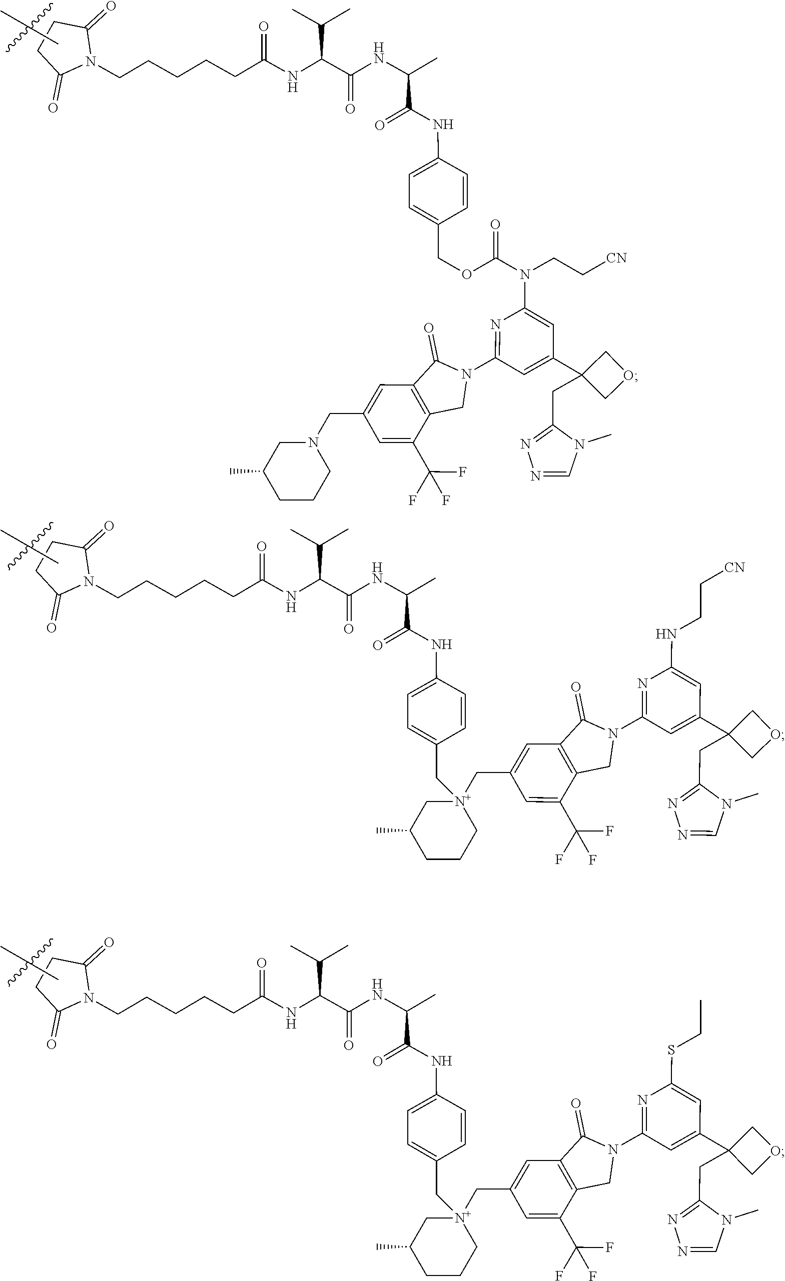 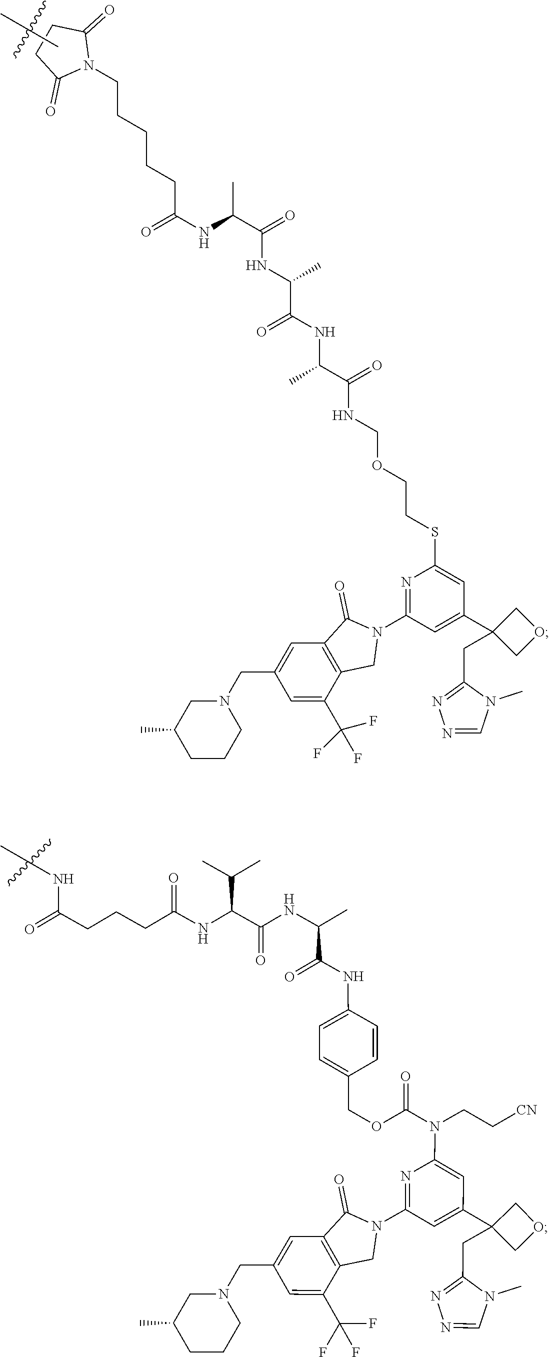 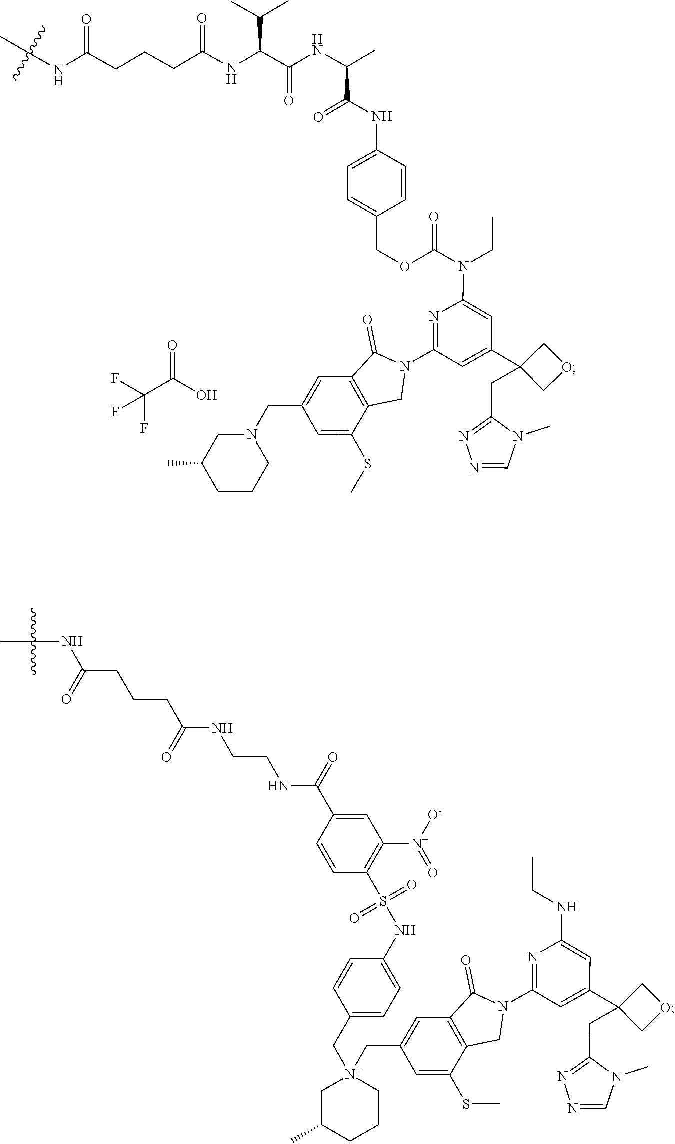
and

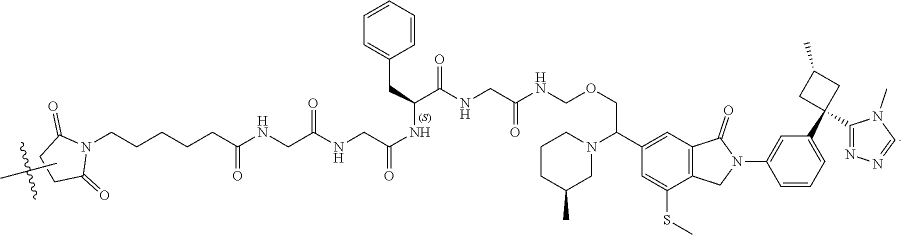

56-58. (canceled)

59. The conjugate of claim 15, or a pharmaceutically acceptable salt thereof, wherein the binding moiety is an antibody or antigen-binding fragment thereof or a small molecule.

60-93. (canceled)

94. A composition comprising the compound of claim 1, or a pharmaceutically acceptable salt thereof.

95. A composition comprising the conjugate of claim 15 or a pharmaceutically acceptable salt thereof.

96. A method of (a) treating cancer in a subject in need thereof or (b) treating a condition that would benefit from an increased immune response in a subject in need thereof, the method comprising administering to the subject the conjugate of claim 15.

97-99. (canceled)

100. A method of (a) increasing the activity of an immune cell; (b) increasing proliferation of an immune cell, (c) increasing migration of an immune cell to a tumor cell, (d) reducing exhaustion of an immune cell, or (e) increasing secretion of IFN-γ or IL-2 from an immune cell, the method comprising contacting the immune cell with the conjugate of claim 15, or a pharmaceutically acceptable salt thereof.

101-106. (canceled)

107. A method of enhancing and/or sustaining an antigen recall response of a T cell comprising contacting the T cell with the conjugate of claim 15 or a pharmaceutically acceptable salt thereof.

108. A method of delivering a payload that is capable of activating an effector T cell to an immune cell, the method comprising contacting the effector T cell with the conjugate of claim 15 or a pharmaceutically acceptable salt thereof.

109-110. (canceled)


SEQUENCE LISTING (28 sequences)

SEQ ID NO: 1 - protein, 272 aa, synthetic construct
  REGION 1..272: note = Human CD25
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 2 - protein, 288 aa, synthetic construct
  REGION 1..288: note = human PD1
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 3 - protein, 240 aa, synthetic construct
  REGION 1..240: note = human CD7
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 4 - protein, 122 aa, synthetic construct
  REGION 1..119: note = VH Antibody A a686 (anti-human CD25)
QVQLVQSGAEVKKPGSSVKVSCKASGGTFSSLAISWVRQAPGQGLEWMGGIIPIFGTANYAQKFQGRVTITADESTSTAYMELSSLRSEDTAVYYCARGGSVSGTLVDFDIWGQGTMVTVSS

SEQ ID NO: 5 - protein, 107 aa, synthetic construct
  REGION 1..107: note = VL Antibody A a686 (anti-human CD25)
DIQMTQSPSTLSASVGDRVTITCRASQSISSWLAWYQQKPGKAPKLLIYKASSLESGVPSRFSGSGSGTEFTLTISSLQPDDFATYYCQQYNIYPITFGGGTKVEIK

SEQ ID NO: 6 - protein, 119 aa, synthetic construct
  REGION 1..119: note = VH Antibody B MA251 (anti-human CD25)
QVQLKESGPGLVAPSQSLSITCTVSGFSLTSYGIQWVRQPPGKGLEWLGVIWAGGSTNYNSALMSRLSISKDNSKSQVFLKMNSLQTDDTAMYYCARAYGYDGSWLAYWGQGTLVTVSS

SEQ ID NO: 7 - protein, 106 aa, synthetic construct
  REGION 1..106: note = VL Antibody B MA251 (anti-human CD25)
QIVLSQSPAILSASPGEKVTMTCRASSSVSYMHWYQQKPGSSPKPWIFATSNLASGVPARFSGSGSGTSYSLTINRVEAEDADTYYCQQWSSNPPTFGGGTKLEIK

SEQ ID NO: 8 - protein, 119 aa, synthetic construct
  REGION 1..119: note = VH Antibody C Human-ized MA251 (anti-human CD25)
QVQLQESGPGLVKPSETLSLTCTVSGFSLTSYGIQWIRQPPGKGLEWLGVIWAGGSTNYNSALMSRLTISKDNSKNQVSLKLSSVTAADTAVYYCARAYGYDGSWLAYWGQGTMVTVSS

SEQ ID NO: 9 - protein, 106 aa, synthetic construct
  REGION 1..106: note = VL Antibody C Human-ized MA251 (anti-human CD25)
DIQLTQSPSSLSASVGDRVTITCRASSSVSYMHWYQQKPGKAPKPWIFATSNLASGVPSRFSGSGSGTDYTLTISSLQPEDFATYYCQQWSSNPPTFGGGTKVEIK

SEQ ID NO: 10 - protein, 120 aa, synthetic construct [sequence omitted]

SEQ ID NO: 11 - protein, 111 aa, synthetic construct [sequence omitted]

SEQ ID NO: 12 - protein, 113 aa, synthetic construct [sequence omitted]

SEQ ID NO: 13 - protein, 107 aa, synthetic construct [sequence omitted]

SEQ ID NO: 14 - protein, 123 aa, synthetic construct
  REGION 1..123: note = VH Antibody H WT1 CS (anti-human CD7)
QIQLVQSGPELKKPGETVKISCKASGYTFTNYGMNWVKQAPGKGLMWLGWINTYTGEPTYADDFKGRFAFSLETSASTAYLQINNLKNEDTATYFCARWAYFYGSSPYFFDYWGQGTTLTVSS

SEQ ID NO: 15 - protein, 109 aa, synthetic construct
  REGION 1..109: note = VL Antibody H WT1 CS (anti-human CD7)
QAVVTQESALTTSPGETVTLTCRSSTGAVTTSNYANWVQEKPDHLFTGLIGGTNNRAPGVPARFSGSLIGDKAALTITGAQTEDEAIYFCALWSSNHLVFGGGTKLTVL

SEQ ID NO: 16 - protein, 114 aa, synthetic construct
  REGION 1..114: note = VH Antibody D 7D4 (anti-mouse CD25)
EVQLQQSGAALVKPGASVKMSCKASGYSFPDSWVTWVKQSHGKSLEWIGDIFPNSGATNFNEKFKGKATLTVDKSTSTAYMELSRLTSEDSAIYYCTRLDYGYWGQGVMVTVSS

SEQ ID NO: 17 - protein, 112 aa, synthetic construct
  REGION 1..112: note = VL Antibody D 7D4 (anti-mouse CD25)
DVVLTQTPPTLSATIGQSVSISCRSSQSLLHSNGNTYLNWLLQRPGQPPQLLIYLASRLESGVPNRFSGSGSGTDFTLKISGVEAEDLGVYYCVQSSHFPNTFGVGTKLEIK

SEQ ID NO: 18 - protein, 121 aa, synthetic construct
  REGION 1..121: note = VH Antibody G J43 (anti-mouse PD1)
EVRLLESGGGLVKPEGSLKLSCVASGFTFSDYFMSWVRQAPGKGLEWVAHIYTKSYNYATYYSGSVKGRFTISRDDSRSMVYLQMNNLRTEDTATYYCTRDGSGYPSLDFWGQGTQVTVSS

SEQ ID NO: 19 - protein, 110 aa, synthetic construct
  REGION 1..110: note = VL Antibody G J43 (anti-mouse PD1)
SYELTQPPSASVNVGETVKITCSGDQLPKYFADWFHQRSDQTILQVIYDDNKRPSGIPERISGSSSGTTATLTIRDVRAEDEGDYYCFSGYVDSDSKLYVFGSGTQLTVL

SEQ ID NO: 20 - protein, 329 aa, synthetic construct
  REGION 1..329: note = IgG1 Heavy Chain Constant Region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 329 aa, synthetic construct
  REGION 1..329: note = IgG1 N297A Constant regions (CH1-Hinge-CH2-CH3)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 326 aa, synthetic construct
  REGION 1..326: note = IgG4 S228P Constant regions (CH1-Hinge-CH2-CH3)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 23 - protein, 329 aa, synthetic construct
  REGION 1..329: note = IgG1 Heavy Chain Constant Region S239C
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 329 aa, synthetic construct
  REGION 1..329: note = IgG1 Heavy Chain Constant Region K334C
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 982 aa, synthetic construct [sequence omitted]

SEQ ID NO: 26 - protein, 449 aa, synthetic construct
  REGION 1..449: note = Heavy Chain Antibody I
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 218 aa, synthetic construct
  REGION 1..218: note = Light Chain Antibody I
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG